(19) United States
(12) Patent Application Publication
(10) Pub. No.: US 2004/0076974 A1
(43) Pub. Date: Apr. 22, 2004

(54) Liver necrosis predictive genes

(76) Inventors: Larry D. Kier, Santa Fe, NM (US); Timothy D. Nolan, West Palm Beach, FL (US); Usha Sankar, Wayne, NJ (US); Maher Derbel, Cambridge, MA (US)

Correspondence Address: ROBERTS ABOKHAIR & MARDULA, SUITE 1000, 11800 SUNRISE VALLEY DRIVE, RESTON, VA, 20191, US

(21) Appl. No.: 10/404,460

(22) Filed: Apr. 1, 2003

Related U.S. Application Data
(60) Provisional application No. 60/369,287, filed on Apr. 1, 2002.

(51) Int. Cl. C12Q 1/68; G06F 19/00; G01N 33/48; G01N 33/50

(52) U.S. Cl. 435/6; 702/20

(57) ABSTRACT

The invention provides toxicity predictive genes that can be used to predict toxicity in response to one more agents. The invention provides for a method of predicting the liver toxicity in an individual to an agent. The method comprises obtaining a biological sample from an individual treated with the agent. The expression of one or more liver toxicity predictive genes in the sample is measured, wherein the genes are selected from a group consisting of partial gene sequences of genes identified as responsive to agents causing liver necrosis. The process generates a test expression profile. The test expression profile is used with a set of reference expression profiles in a Predictive Model to determine whether the agent will induce liver toxicity in the individual.

### CROSS REFERENCE TO OTHER PATENT APPLICATIONS

This application claims priority to U.S. provisional application 60/369,287 filed Apr. 01, 2002, which is hereby incorporated by reference in its entirety.

### REFERENCE TO A SEQUENCE LISTING AND TABLES

This application contains a gene sequence listing and 4 tables submitted on a compact disc whose file name is “2874-022US” created on Apr. 01, 2003 containing 4 files and is herein incorporated by reference in its entirety. The files are: a) Table 32.xls, 214 KB, b) Table 34.xls, 525 KB, c) Table 35.xls, 626 KB, and d) Table 36.xls 576 KB, all in Microsoft Excel™.

The contents of the files contained on the CD-ROM discs submitted with this application are hereby incorporated by reference into the specification.

### BACKGROUND

This invention is the field of toxicology. More specifically, it relates to toxicity predictive genes and the methods of using such genes to predict toxicity.

Molecular biology and genomics technologies have potential to create dramatic advances and improvements for the science of toxicology as for other biological sciences. See, for example, MacGregor, et al. Fund. Appl. Tox. 26:156-173, 1995; Rodi et al., Tox. Pathology 27:107-110, 1999; Cunningham et al., Ann. N.Y. Acad. Sci. 919: 52-67, 2000; Pritchard et al.,Proc. Nat. Acad. Sci. USA 98:13266-13271, 2001; and Fielden and Zacharewski, Tox. Sciences 60: 6-10, 2001.

The advantage of these technologies is that they can provide massive amounts of parallel information and that this information concerns processes and events occurring at the molecular level. This level of information is in dramatic contrast to conventional safety assessment toxicology that, to a large extent, currently relies on subjective evaluation (e.g., in-life observations of behavior, observations of gross abnormalities at necropsy and histopathological examination of stained tissue slides using a microscope). These current methodologies may be largely subjective and in some cases such as histopathological evaluation, they require someone with a high degree of training, experience and skill to make competent evaluations. Furthermore, many of the methodologies require access to organs and tissues that necessitates either killing laboratory animals or surgery to obtain tissue specimens.

Recently, there have been some initial efforts to apply molecular biology and genomics technologies to toxicology. Some efforts have involved application of gene expression measurements. See, for example, U.S. Pat. No. 6,228,589 and WO 01/05804. Analysis of the data has yielded interesting observations of gene expressions that appear to correlate with some toxic effects or mechanisms. See, for example, Mueller et al. Environmental Health Perspectives 106(5): 277-230 (1998). However, there has been very little published work in toxicology so far that applies rigorous analytical and statistical techniques to the massive amounts of data available from genomics technologies. The observations, so far, have tended to be phenomenological and focused on individual gene responses rather than determining the generally applicable capabilities of patterns of gene expression to predict toxic effects (see, for example, studies of gene expression altered by exposure to toxicants in Bartosiewicz et al., Environ health Perspectives 109:71-74, 2001; Huang et al., Tox. Sciences 63: 196-207, 2001). Even in the larger field of biological sciences, these types of analyses are just beginning to be evidenced in the literature (e.g., Golub et al., Science 286: 531-537, 1999).

U.S. Pat. No. 6,228,589 (Brenner) shows a method for assessing the toxicity of a compound in a test organism by measuring gene expression profiles of selected tissue.

Recently some work has been published that attempts to correlate gene expression profiles with the mechanism of toxicity of various hepatotoxins. See for example, Waring et al. Tox. and Appl. Pharm. 175:28-42 (2001). However there has been limited success thus far in the attempts to predict toxicity of compounds based on the gene expression profiles elicited upon treatment.

What is needed are genes and predictive models, which are capable of predicting toxicity response.

### SUMMARY

The invention provides toxicity predictive genes and predictive models that are useful to predict toxic responses to one or more agents.

One aspect of the present invention provides methods of predicting toxicity in an individual to an agent. One method includes the steps of: (a) obtaining a biological sample from an individual treated with the agent or treating a biological sample obtained from an individual with the agent or treating in vitro cultured cells or explants with the agent; (b) obtaining a gene expression profile on one or more of the toxicity predictive genes disclosed herein from the biological sample or in vitro cultured cells or explants; and (c) using the gene expression profiles from the biological sample or cells treated with the agent as a test set and a database of gene expression profiles and toxicity classifications as a training set and using toxicity predictive genes and a Predictive Model to assay whether the agent will induce liver toxicity in the individual or would be predicted to produce liver toxicity following in vivo exposure.

Another aspect of the present invention provides that the predictive model utilizes expression profiles from sets of toxicity predictive gene(s) selected from Combination 5, infra, wherein the set is one or more toxicity predictive gene(s). In other aspects, the predictive model utilizes expression profiles from sets of one or more toxicity predictive gene(s) selected from Combination 4, 3, 2, or 1, wherein the set is one or more toxicity predictive gene(s).

Yet another aspect of the present invention provides methods for determining the presence or absence of a no-observable effect level (NOEL) of an agent in an individual. One method includes the steps of: (a) obtaining biological samples from individuals treated with the agent at different dose levels or treating a biological sample obtained from, an individual with different dose levels of the agent or treating a biological sample obtained from an individual with different dose levels of the agent or treating in vitro cultured cells or explants with different dose levels of the agent; (b) obtaining gene expression profiles of the samples; and (c) using the gene expression profile from the biological samples as a test set and a database of gene expression profiles and toxicity classifications as a training set and using toxicity predictive genes and a Predictive Model to determine or predict whether and at which dose levels the agent will induce toxicity.

Another aspect of the present invention provides that the predictive model utilizes sets of toxicity predictive gene(s) selected from Combination 5, wherein the set is one or more toxicity predictive gene(s). In other aspects, the predictive model utilizes sets of toxicity predictive gene(s) selected from Combination 4, 3, 2, or 1, wherein the set is one or more toxicity predictive gene(s).

A further aspect of the present invention provides that the predictive genes and models may be used with an in vitro system to identify in vitro systems that can be used to accurately predict in vivo toxicity and to use the identified in vitro systems to accurately predict in vivo toxicity.

Another aspect of the present invention provides methods of identifying toxicity predictive genes. One method includes the steps of: (a) providing a set of candidate toxicity predictive genes; (b) evaluating the genes for their predictive performance with at least one training and test set of data in a Predictive Model to identify genes which are predictive of toxicity; and (c) testing the performance of predictive genes for their ability to predict toxicity for different training and test sets of data, for prediction of accurate compared to random classification and prediction of test data external to the data used to derive the predictive genes. A further embodiment provides the candidate toxicity predictive genes are rat toxicity genes.

Yet another aspect of the present invention provides a computer-based method for mining genes predictive for toxicity. One method includes the steps of collecting expression levels of a plurality of candidate toxicity predictive genes in a multiplicity of samples; optionally storing the expression levels as a database on an electronic medium; defining a group of samples to be a training set; defining another group of samples to be a test set; optionally generating additional training and test sets; and selecting a set of genes which are predictive of toxicity based on evaluating the training set and the test set in a Predictive Model.

In another aspect, the invention provides a computer program product for predicting toxicity that includes a set of toxicity predictive genes derived from mining a database having a plurality of gene expression profiles indicative of toxicity. In a further aspect, the set of toxicity predictive genes includes at least one toxicity predictive gene from combination 5, 4, 3, 2, or 1 list.

In another aspect, the invention provides a library of expression profiles of toxicity predictive genes produced by the methods disclosed herein.

In another aspect, the invention provides an integrated system for predicting toxicity including equipment capable of measuring gene expression profiles of toxicity predictive genes from biological samples exposed to a test agent, operably linked to a computer system capable of implementing a predictive model.

### BRIEF DESCRIPTION OF THE DRAWINGS

FIG. 1 is a flow diagram illustrating one embodiment of the present invention for identification of toxicity predictive genes.

FIG. 2 is a flow diagram illustrating one embodiment of the present invention for evaluating performance of toxicity predictive genes.

FIG. 3 is a flow diagram illustrating one embodiment of the present invention for using toxicity predictive genes to predict toxicity.

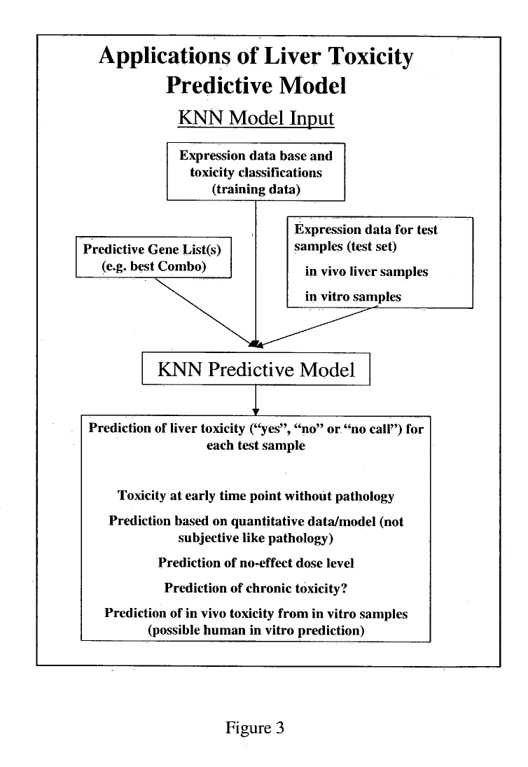

FIG. 4 is a graph that illustrates one embodiment of the present invention showing the percent of overall correct calls as a function of number of predictor genes—histopathology correlating genes (Pearson correlation measure) with training and test set 3. The percent of overall correct calls is presented as a function of the number of predictor genes. The input genes list was a list of 61 genes that correlated with histopathology scores using Pearson's correlation measure (r-value>0.45).

Training and Test Set 3 was used with other model values of 10 nearest neighbors and a p-value ratio cutoff of 0.5. An optimum gene number of 9 was observed (lowest number of genes giving the highest percent overall calls) for this case.

FIG. 5 is a graph that illustrates K Means and Tree Clustering for Combo 5, 4, 3, 2 Genes. Cluster patterns are shown for an 8 cluster analysis of predictive genes from the Combo 5, 4, 3, and genes that corresponds to one embodiment of the invention. The individual genes located in each of the 8 clusters are presented in Table 30.

### BRIEF DESCRIPTION OF THE TABLES

Table 1 lists compounds, dose levels, pathology and abbreviations in the database in accordance with one embodiment of the present invention.

Table 2 lists distribution of compounds in individual training and test sets for 24 hour data in accordance with one embodiment of the present invention.

Table 3 lists gene whose expression at 24 hour directly correlates with necrosis at 72 hour, ranked by Pearson correlation coefficient in accordance with one embodiment of the present invention.

Table 4 lists genes whose expression at 24 hour inversely correlates with necrosis at 72 hour, ranked by Spearman correlation coefficient in accordance with one embodiment of the present invention.

Table 5 lists predictive genes for 24 hour expression data in accordance with one embodiment of the present invention.

Table 6 lists randomly selected gene subsets from 24 hour Combo All gene set in accordance with one embodiment of the present invention.

Table 7 lists randomly selected gene subsets from 24 hour Combos 5, 4, 3 combined in accordance with one embodiment of the present invention.

Table 8 lists randomly selected gene subsets from 24 hour all excluding predictive genes (i.e., excluding Combo All genes) in accordance with one embodiment of the present invention.

Table 9 lists toxicity individual sample prediction values for 24 hour data predictive genes (combined list and subsets) in accordance with one embodiment of the present invention.

Table 10 lists toxicity compound-dose prediction values for 24 hour data predictive genes (combined list and subsets) in accordance with one embodiment of the present invention.

Table 11 lists toxicity compound prediction values for 24 hour data predictive genes (combined list and subsets) in accordance with one embodiment of the present invention.

Table 12 lists individual gene predictions for Combo 5 in accordance with one embodiment of the present invention.

Table 13 lists individual gene predictions for Combo 4 in accordance with one embodiment of the present invention.

Table 14 lists individual gene predictions for Combo 3 in accordance with one embodiment of the present invention.

Table 15 lists toxicity compound-dose prediction values for 24 hour data with random gene subsets in accordance with one embodiment of the present invention.

Table 16 lists comparison of predictivity for correct toxicity classification and random classification using Combo gene sets and random subsets and 24 hour data in accordance with one embodiment of the present invention.

Table 17 lists distribution of compounds in individual training and test sets for 6 hour data in accordance with one embodiment of the present invention.

Table 18 lists genes whose expression at 6 hours directly correlates with hepatocellular necrosis at 72 hours, ranked by Pearson correlation coefficient in accordance with one embodiment of the present invention.

Table 19 lists genes whose expression at 6 hours inversely correlates with necrosis at 72 hours, ranked by Spearman correlation coefficient in accordance with one embodiment of the present invention.

Table 20 lists genes whose expression at 6 hours is predictive of toxicity at 72 hours in accordance with one embodiment of the present invention.

Table 21 lists toxicity compound-dose prediction values for 6 hour data predictive genes (combined list and subsets) in accordance with one embodiment of the present invention.

Table 22 lists comparison of predictivity for correct toxicity classification and random classification using combo gene sets 6 hour data in accordance with one embodiment of the present invention.

Table 23 lists distribution of compounds in individual training and test sets for 72 hour data in accordance with one embodiment of the present invention.

Table 24 lists genes whose expression at 72 hours directly correlates with necrosis at 72 hours, ranked by Pearson correlation coefficient in accordance with one embodiment of the present invention.

Table 25 lists genes whose expression at 72 hours inversely correlates with necrosis at 72 hours, ranked by Spearman correlation coefficient in accordance with one embodiment of the present invention.

Table 26 lists genes whose expression at 72 hours is predictive of toxicity at 72 hours in accordance with one embodiment of the present invention.

Table 27 lists toxicity compound-dose prediction values for 72 hour data predictive genes (combined list and subsets) in accordance with one embodiment of the present invention.

Table 28 lists comparison of predictivity for correct toxicity classification and random classification using combo gene sets 72 hour data in accordance with one embodiment of the present invention.

Table 29 lists prediction of toxicity for samples external to database in accordance with one embodiment of the present invention.

Table 30 lists K-means cluster analysis of combo 5, 4, 3 and 2 gene set in accordance with one embodiment of the present invention.

Table 31 lists RCT genes (ESTs) predictive for necrosis at 72 hours: best homology matches in accordance with one embodiment of the present invention.

Table 32 lists genes predictive for necrosis, sequences, and accession numbers in accordance with one embodiment of the present invention.

Table 33 lists hepatocellular necrosis predictive genes whose protein products are known to be secreted. The genes are from the table listing hepatocellular necrosis predictive genes at the three time points 6, 24 and 72 hours. The protein products are easier to access since they are secreted into body fluids and are thus more amenable to be quantified. Therefore these proteins can be monitored in body fluids of subjects such as humans and toxicity predictions can be made.

Table 34 lists expression data for the 6 hour timepoint in accordance with one embodiment of the present invention.

Table 35 lists expression data for the 24 hour timepoint in accordance with one embodiment of the present invention.

Table 36 lists expression data for the 72 hour timepoint in accordance with one embodiment of the present invention.

Table 37 lists predictive performance of predictive genes organized by occurrence on training/test set lists (combo number) and time point in accordance with one embodiment of the present invention.

Table 38 lists 266 liver toxicity predictive genes organized by time point and combo class in accordance with one embodiment of the present invention.

Table 39 lists Liver Predictive genes that are predictive across all three time points in accordance with one embodiment of the present invention.

Table 40 lists Liver Predictive genes that are most predictive across all three time points in accordance with one embodiment of the present invention.

### DETAILED DESCRIPTION

One embodiment of the present invention provides for a method of predicting the liver toxicity in an individual to an agent. The method comprises obtaining a biological sample from an individual treated with the agent. The expression of one or more liver toxicity predictive genes in the sample is measured, wherein the genes are selected from a group consisting of partial gene sequences of genes identified as responsive to agents causing liver necrosis. The process generates a test expression profile. The test expression profile is used with a set of reference expression profiles in a Predictive Model to determine whether the agent will induce liver toxicity in the individual.

Another embodiment of the present invention provides for a method of predicting the liver toxicity of an agent. The method comprising using an in vitro system which comprises obtaining a biological sample from an in-vitro cultured cells or explants treated with the agent. The expression of one or more liver toxicity predictive genes in the sample is measured. The genes are selected from a group consisting of partial gene sequences of genes identified as responsive to agents causing liver necrosis. The process generates a test expression profile. The test expression profile is used with a set of reference expression profiles in a Predictive Model to determine whether the agent will induce liver toxicity in the individual.

Yet another embodiment of the present invention provides for a process for predicting the liver toxicity in a biological sample from an individual, in-vitro cell cultures or explants to an agent via a programmable machine. The process comprises obtaining a biological sample treated with the agent. The expression of one or more liver toxicity predictive genes in the sample is measured. The genes are selected from a group consisting of partial gene sequences of genes identified as responsive to agents causing liver necrosis. The steps generate a test expression profile. The test expression profile is used with a set of reference expression profiles in a Predictive Model to determine whether the agent will induce liver toxicity in the individual.

Still another embodiment of the present invention provides a computer program product for enabling a computer to perform Predictive Model analysis for liver toxicity on a biological sample from an individual, in-vitro cell cultures or explants to an agent. The computer program product comprises software instructions enabling the computer to perform predetermined operations, and a computer readable medium embodying the software instructions. The predetermined operations comprise measuring an expression of one or more liver toxicity predictive genes in a sample, wherein the genes are selected from a group consisting of partial gene sequences of genes identified as responsive to agents causing liver necrosis. A test expression profile is thus generated. The test expression profile is used with a set of reference expression profiles in a Predictive Model to determine whether the agent will induce liver toxicity in the individual.

Yet a further embodiment of the present invention provides a Computer system adopted to predict liver toxicity in a biological sample from an individual, in-vitro cell cultures, or explants to an agent. The computer system comprising a processor and a memory including software instructions adapted to enable the computer system to perform operations. The software instructions comprising measuring the expression of one or more liver toxicity predictive genes in the sample, wherein the genes are selected from the group consisting of partial gene sequences of genes identified as responsive to agents causing liver necrosis, thereby generating a test expression profile; and using the test expression profile with a set of reference expression profiles in a Predictive Model to determine whether the agent will induce liver toxicity in the individual.

A further embodiment of the present invention provides, a computer program product for predicting liver toxicity from a test sample expression profile. The computer program product comprises an encrypted training data set; encrypted lists of genes selected from genes predictive of liver toxicity to be used with the encrypted training data set, and a Predictive Model that uses the encrypted training data sets, the encrypted lists of genes, and the test sample expression profile to predict the liver toxicity of the test sample.

Another embodiment of the present invention provides a method for mining genes predictive for liver toxicity. The method comprises collecting expression levels of a plurality of candidate toxicity predictive genes among a multiplicity of samples. A group of samples are defined as a training set. Another group of samples are defined to be a test set. Optionally, additional training and test sets are generated. A set of genes which are predictive of liver toxicity are selected based on evaluating the training and test sets in a Predictive Model.

This invention relates to methods of predicting whether an agent or other stimulus is capable of inducing toxicity in a. recipient organism using predictive molecular toxicology analysis. In particular, the invention provides methods of predicting toxicity which comprise analyzing gene and/or protein expression profiles across a number of toxicity biomarkers disclosed herein for patterns of expression that are predictive of toxicity in the recipient organism. This type of toxicity is significant as a toxic effect of many chemical agents and is a significant component of adverse reactions to pharmaceuticals and drugs (see, for example, Treinen-Moslen, M. in Casarett and Doull's Toxicology: The Basic Science of Poisons Sixth Edition (C. D. Klaasen, ed.) Chapter 13, McGraw-Hill, New York, 2001). The invention is based, in part, upon the discovery that modulated transcriptional regulation of relatively small sets of certain genes in response to a test agent can accurately predict the occurrence of toxicity observed at later time points.

Provided herein are multiple sets of toxicity biomarkers which are useful in the practice of the toxicity prediction methods of the invention. In particular, Applicants have identified 266 toxicity biomarkers that demonstrate utility in predicting toxicity outcomes. These biomarkers have been thoroughly characterized for their predictive performance, individually as well as in various combinations or subsets thereof. In addition, various optimized subsets of the toxicity biomarkers of the present invention are disclosed. These sets have also been thoroughly characterized for predictive performance using the methods of the invention. Among the subsets of toxicity genes provided herein are several which demonstrate prediction accuracies in the vicinity of 90%.

The present invention is further described by way of the experimental examples provided herein. These examples demonstrate that small sets of genes (i.e., in some instances, as few as 1, 2 or 3 biomarker genes) are used to accurately predict toxicity. For example, as further described in the Examples, analysis of mRNA expression of only a few genes provides an accurate indication of whether a test agent will or will not induce toxicity.

The predictive capacity of the methods of the invention have been verified by comparisons with random classifications, and data derived external to the database used to identify toxicity biomarkers. Moreover, the methods of the invention are capable of distinguishing between agent dose levels that induce toxicity (typically higher doses) and those doses that are non-toxic. This latter feature is an important component of meaningful toxicological evaluation.

The practice of the present invention will employ, unless otherwise indicated, conventional techniques of molecular biology (including recombinant techniques), microbiology, cell biology, biochemistry, nucleic acid chemistry, and immunology, which are well known to those skilled in the art. Such techniques are explained fully in the literature, such as, Molecular Cloning: A Laboratory Manual, second edition (Sambrook et al., 1989) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2001); PCR: The Polymerase Chain Reaction, (Mullis et al., eds., 1994); Harlow and Lane (1988) Antibodies, A Laboratory Manual, Cold Spring Harbor Publications, New York; Harlow and Lane (1999) Using Antibodies: A Laboratory Manual Cold Spring Harbor Laboratory Press, Cold Spring Harbor, N.Y. (jointly referred to herein as “Harlow and Lane”), Beaucage et al. eds., Current Protocols in Nucleic Acid Chemistry John Wiley & Sons, Inc., New York, 2000) and Casarett and Doull's Toxicology The Basic Science of Poisons, C. Klaassen, ed., 6^{th} edition (2001).

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 2^{nd} edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

“Toxic” or “toxicity” refers to the result of an agent causing adverse effects, usually by a xenobiotic agent administered at a sufficiently high dose level to cause the adverse effects.

As used herein, the terms “toxicity biomarker” and “toxicity predictive gene” are used interchangeably and refer to a gene whose expression, measured at the RNA or protein level can predict the likelihood of a toxicity response with accuracy significantly better than would occur by chance. toxicity response can be necrosis or any other toxicity manifestations that elicit similar detectable gene expression changes. These could include, but are not limited to, other forms of pathology such as centrilobular hepatocellular vacuolar degeneration, apoptosis, inflammation and cirrhosis.

A “toxicological response” or “toxicity response” refers to a cellular, tissue, organ or system level response to exposure to an agent. At the molecular level, this can include, but is not limited to, the differential expression of genes encompassing both the up- and down-regulation of expression of such genes at the RNA and/or protein level; the up- or down-regulation of expression of genes which encode proteins associated with response to and mitigation of damage, the repair or regulation of cell damage; or changes in gene expression due to changes in populations of cells in the tissue or organ affected in response to toxic damage.

An “agent” or “compound” is any element to which an individual can be exposed and can include, without limitation, drugs, pharmaceutical compounds, household chemicals, industrial chemicals, environmental chemicals, other chemicals, and physical elements such as electromagnetic radiation.

The term “biological sample” as used herein refers to substances obtained from an individual. The samples may comprise cells, tissue, parts of tissues, organs, parts of organs, or fluids (e.g., blood, urine or serum). Biological samples include, but are not limited to, those of eukaryotic, mammalian or human origin.

“Sample” is defined for the purposes of prediction as a biological sample and the gene expression data for that sample. Each sample may come from an individual animal. A toxicity classification may also be associated with the sample.

“Gene expression” as used herein refers to the levels of expression and/or pattern of expression of a gene.

“Gene expression profile” refers to the levels of expression of multiple different genes measured for the same sample. Gene expression profiles may be measured in a sample, such as samples comprising a variety of cell types, different tissues, different organs, or fluids (e.g., blood, urine, spinal fluid, sweat, saliva or serum) by various methods including but not limited to microarray technologies and quantitative and semi-quantitative RT-PCR (e.g., Taqman™) techniques, as well as techniques for measuring expression of proteins.

“Individual” refers to a vertebrate, including, but not limited to, a human, non-human primate, mouse, hamster, guinea pig, rabbit, cattle sheep, pig, chicken, and dog.

As used herein, the terms “hybridize”, “hybridizing”, “hybridizes” and the like, used in the context of polynucleotides, are meant to refer to conventional hybridization conditions, such as hybridization in 50% formamide/6× SSC/0.1% SDS/100 μg/ml ssDNA, in which temperatures for hybridization are above 37 degrees Celsius and temperatures for washing in 0.1× SSC/0.1% SDS are above 55 degrees Celsius, and preferably to stringent hybridization conditions. The hybridization of nucleic acids can depend upon various factors such as their degree of complementarity as well as the stringency of the hybridization reaction conditions. Stringent conditions can be used to identify nucleic acid duplexes with a high degree of complementarity. Means for adjusting the stringency of a hybridization reaction are well known to those of skill in the art. See, for example, Sambrook, et al., “Molecular Cloning: A Laboratory Manual,” Second Edition, Cold Spring Harbor Laboratory Press, 1989; Ausubel, et al., “Current Protocols In Molecular Biology,” John Wiley & Sons, 1996 and periodic updates; and Hames et al., “Nucleic Acid Hybridization: A Practical Approach,” IRL Press, Ltd., 1985. In general, conditions that increase stringency (i.e., select for the formation of more closely matched duplexes) include higher temperature, lower ionic strength and presence or absence of solvents; lower stringency is favored by lower temperature, higher ionic strength, and lower or higher concentrations of solvents.

In the context of amino acid sequence comparisons, the term “identity” is used to express the percentage of amino acid residues at the same relative position that are the same. Also in this context, the term “homology” is used to express the percentage of amino acid residues at the same relative positions which are either identical or are similar, using the conserved amino acid criteria of BLAST analysis, as is generally understood in the art. Further details regarding amino acid substitutions, which are considered conservative under such criteria, are provided below.

The toxicity biomarkers described herein were initially identified utilizing a database generated from large numbers of in vivo experiments, wherein the differential expression of approximately 700 rat genes, measured at various time points, in response to multiple toxic compounds inducing various specific toxic responses, as visualized through microscopic histopathological analysis, was quantified, as described in pending U.S. patent application filed Jan. 29, 2002 (Ser. No. 10/060,893). This quantitative gene expression data, as well as corresponding histopathological information, were then subjected to an analytical approach specifically designed to identify genes which not only correlated with the observed histopathology, but also demonstrated an ability to be used in a model capable of accurately predicting the occurrence of the toxic response associated with the observed histopathology. A detailed description of an identification process is presented in the Examples.

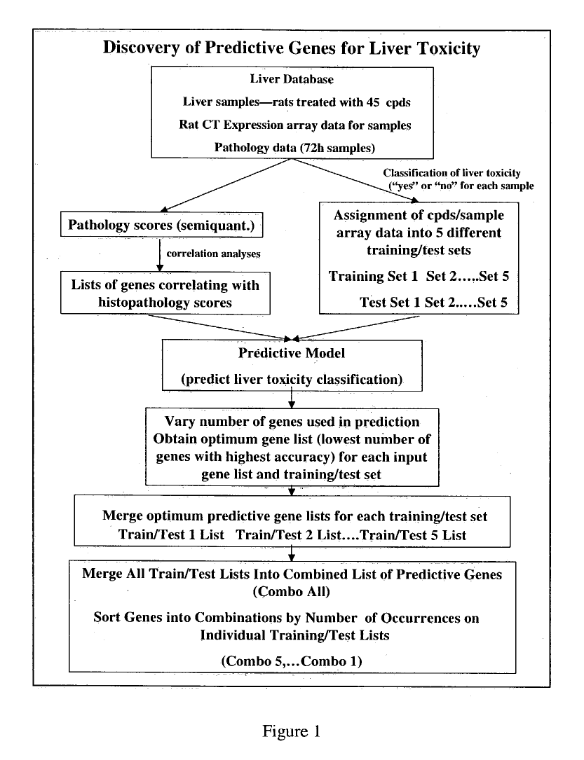

A flow diagram illustrating how the toxicity biomarkers of the invention were identified is illustrated in FIG. 1. In addition to the database described and utilized herein, other toxicology gene expression databases may be generated, and used to identify additional toxicity biomarkers, which may also be employed in the practice of the toxicity prediction methods of the invention. Such databases may be generated with test compounds capable of inducing various pathologies indicative of a toxic response in the and/or other organs or systems, over different time periods and under different administration and/or dosing conditions, including without limitation necrosis, centrilobular hepatocellular vacuolar degeneration, apoptosis, inflammation and cirrhosis. An example of compounds, dose levels, toxicity classifications and histopathology scores used in the Examples that follow is provided in Table 1. Such databases may be generated using organisms other than the rat, including without limitation, animals of canine, murine, or non-human primate species. In addition, such databases may incorporate data derived from human clinical trials and post-approval human clinical experiences. Various methods for detecting and quantitating the expression of genes and/or proteins in response to toxic stimuli may be employed in the generation of such databases, as are generally known in the art. For example, microarrays comprising multiple cDNAs or oligonucleotide probes capable of hybridizing to corresponding transcripts of genes of interest may be used to generate gene expression profiles. Additionally, a number of other methods for detecting and quantitating the expression of gene transcripts are known in the art and may be employed, including without limitation, RT-PCR techniques such as TaqMan®, RNAse protection, branched chain, etc.

Databases comprising quantitative gene expression information preferably include qualitative and quantitative and/or semi-quantitative information respecting the observed toxicological responses and other conventional toxicology endpoints, such as for example, body and organ weights, serum chemistry and histopathology observations, histopathology scores and/or similar parameters.

For the purpose of identifying candidate predictive genes, the database preferably includes histopathology scores for each animal that has been exposed to one or more agent(s). These scores can be assigned based on actual histopathology observations for the tissue and animal or on the basis of effects observed for other animals treated with the same agent and dose level. The scores are numerical scores that reflect the occurrence and severity of histopathological changes. These scores can be adjusted to have similar range to gene expression changes. For example, a score of 1 could be assigned to samples with no changes and scores of 2-8 assigned to increasingly severe changes. Because the scores are numerical, they are suitable for use with a variety of statistical correlation and similarity measures.

An example of a histopathology scoring system is provided in Example 1. Referring to FIG. 1, histopathology scores may be utilized to identify genes which correlate with the observed toxicological response, using any number of statistical correlation and similarity analysis techniques, including without limitation those correlation or similarity measures described or employed in Example 1 (e.g., Pearson, Spearman, change, smooth, distance etc.). Such correlating genes may be used as predictive gene candidates. Examples of genes whose expression at 24 hours after treatment correlates with histopathology observed at 72 h are detailed in Tables 3 and 4. In one embodiment, the correlating gene lists as well as the entire array gene list are used as input gene lists in the GeneSpring™ Predictive Model (otherwise known hereafter as “Predictive Model”).

Statistical analysis of the database of gene expression profiles can be effected by utilizing commercially available software programs. In one embodiment, GeneSpring™ (Version 4.1, Silicon Genetics, Redwood City, Calif.) is used. Other software programs that can be used for statistical analysis are SAS software packages (SAS Institute Inc., Cary, N.C.) and S-PLUS(D software (Insightful Corporation, Seattle, Wash.).

Using GeneSpring™ software, class predictions can be made from the genes in the database, as detailed in Example 1, using one or more training and test sets. In one embodiment, five training sets and five test sets are obtained, as shown in Example 1 (Table 2). Toxicological classifications are entered for the samples in each training and test set. Toxicological classifications can be defined by various pathologies. In one embodiment, the toxicity is defined as necrosis observed 72 hours after treatment with an agent. However, toxicity can manifest in other pathologies such as centrilobular hepatocellular vacuolar degeneration, apoptosis, inflammation and cirrhosis.

Once the training sets have been selected, then predicted classifications of the test set samples are obtained by using k-nearest neighbor (or knn) voting procedure. The class in which each of the knn is determined and the test sample is assigned to the class with the largest representation after adjusting for the proportion of classifications in the training set. In one embodiment, adjustments are made to account for different proportions of classes in the training set.

Toxicity can also be observed at various time points after exposure to an agent and is not limited to only 72 hour after treatment. A skilled toxicologist can determine the optimal time after exposure to an agent to observe pathology by either what has been disclosed in the art or a stepwise experimentation with time increments, for example 2, 4, 6, 12, 18, 24, 36, 48 hours post-exposure or even longer time increments, for example, days, weeks, or months after exposure to the agent.

FIG. 1 describes the overall process used to identify toxicity predictive genes. In one embodiment, this process was run independently for each time point.

The number of genes that are to be used in the Predictive Model can be varied, for example 50, 40, 30, 20, 10, 5, 2, or 1 gene(s) can be used. In a preferred embodiment, at least 50 genes are used.

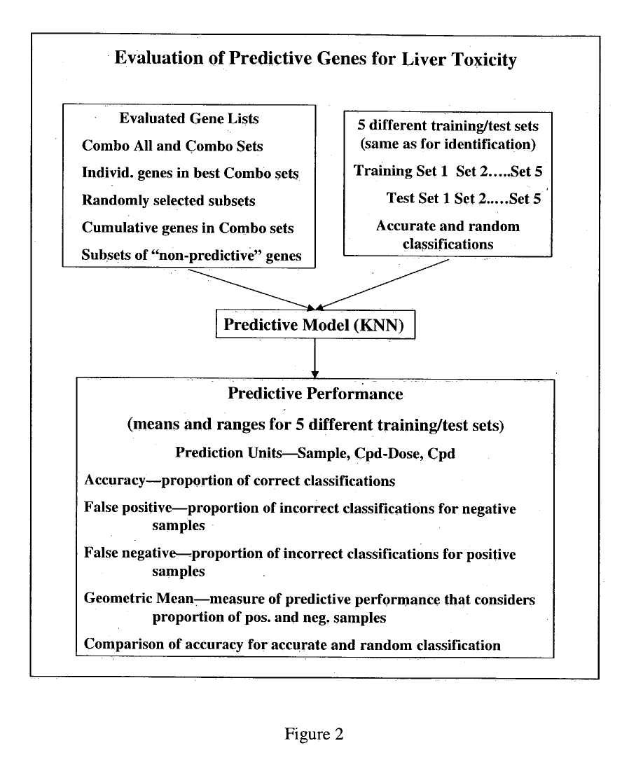

An optimal gene list is generated that generates the best predictive accuracy with the lowest number of genes used. FIG. 2 shows an exemplary profile for an optimal gene list.

Another embodiment of the present invention provides optimum gene lists for all input gene lists are combined for each training and test set and then these combined lists for six training and test sets are merged to create an aggregate list of predictive genes. The aggregate list can then be subdivided to smaller lists of genes based on the number of times that the genes occurred on the predictive gene lists for an individual training or test set. These are designated herein as Combo 5, 4, 3, 2, or 1 lists. The genes that were predictive in 5 training and test sets are designated as Combo 5 and the genes that were predictive in 4 of 5 training and test sets are designated as Combo 4 and so forth. Table 32 presents gene names, accession numbers and sequence information for the toxicity predictive genes found by analysis of the database in the manner described above. Each of these genes has been demonstrated to contribute to predictive performance for at least one input gene list and training/test set and one time point. Table 38 lists the toxicity predictive genes organized by time point and Combo Class. Table 31 lists homologous genes for the RCT sequences that were identified by BLAST search using the GenBank NR database as the target database.

The predictive genes can also be categorized by their occurrence as predictive at different time points. Table 39 lists genes that are on the combined predictive lists of three time points tested. This list is derived from the list of the predictive genes measured at 6, 24 and 72 hours that predicted necrosis at 72 hours. Genes that are predictive at multiple time points can be further grouped by their Combo ranking. Table 40 lists genes that are the most predictive across the three time points tested. This list is a subset of the list of 9 genes that are predictive across three time points 6, 24 and 72 hours. The criteria for inclusion in this table were that the gene be a member of the highest combinations, viz., combinations 5 or 4 in at least 2 out of three time points. The gene expression data of the genes in Table 40 could be expected to be very highly predictive of necrosis. Further, since the predictive strength of these genes is very high across the 3 time points tested, it could be expected that gene expression data derived from these genes even at time points not tested such as any time points falling between 6 and 72 hours or any other time point would be very highly predictive of necrosis. These specific genes could be useful in cases where the dose route or pharmacokinetic properties of a compound may alter the kinetics of predictive gene expression changes.

The predictive genes are evaluated for predictive performance as illustrated in FIG. 2. For each gene list prediction, a table of data is generated using the Predictive Model which includes: the test set containing information about the actual call (i.e., “yes” or “no” for toxicity), the predicted call (i.e., “yes” or “no” for toxicity), and the P-value cutoff ratio. Expression data that can be used with the K-nearest neighbor model and predictive genes to enable one skilled in the art to make predictions are given in Tables 34-36.

The combined list of predictive genes or alternatively, Combo 5, 4, 3, 2, or 1 list or subsets thereof is used as input into the Predictive Model. As an external verification of the predictive abilities of the genes found to be predictive for toxicity, random lists of genes may be generated and also used as input into the Predictive Model. Example 2 describes the evaluation of the predictive performance of the toxicity predictive genes.

Predictive performance may also be assessed using data from different time points after exposure to the agent. In one embodiment, 24 hour expression data is used. In another embodiment, 6 hour expression data is used, as described in Examples 3 and 4. In another embodiment, 72 hour expression data is used, as described in Example 5 and 6. As shown in Table 37, predictive capability for 24 hour expression data has a high accuracy rate (i.e., 92% accuracy) when the entire predictive gene list is used.

Somewhat lower predictive accuracies were observed for the 6 h and 72 h data but the prediction was still quite significant. All of the combo lists as well as Combo All list had significantly higher accuracy than using random classifications.

Predictive performance may also be assessed using subsets of genes from the different Combo lists. As indicated in Examples 2, 4 and 6 randomly selected subsets of the Combo gene lists had very good predictive performance (accuracy better than 80% and approaching 90% and even individual genes had mean predictive accuracies that were significant (for example, greater than 80%). In one embodiment, using 5 genes from Combo All yields about 89% accuracy. Using different Combo lists may require a greater number of genes to reach the same accuracy level.

The toxicity predictive genes disclosed herein and toxicity predictive genes identified by using methods disclosed herein are useful for predicting toxicity in response to exposure to one or more agents.

The discovery that relatively small sets of different genes have predictive value permits flexible applications. The choice of how many and which genes to use can be tailored to a variety of different purposes. Very good predictivity is observed for sets of a few genes (for example, 24 hour Combo 5 which has only 3 genes had a mean prediction accuracy of about 90%). These small sets may be particularly advantageous in applications where measurement of only a few RNA species has considerable advantages in terms of sample processing logistics, speed and cost. These applications would include relatively high throughput screens for predictive capability. An example of this would be an early screen using small samples of primary cells or cultured cell lines that can be processed with automated robotic equipment for treatment and isolation of RNA followed by efficient technologies for measuring expression of a few RNA species such as branched chain technology or RT-PCR.

The use of larger numbers of predictive genes provides redundancy that may improve accuracy and precision. Applications using larger numbers of predictive genes might be tests of candidates at later stages of commercial development. An example would be later stages of preclinical development of a therapeutic candidate where in vivo samples can be obtained and more comprehensive methods such as microarray measurement of gene expression are appropriate. The larger gene sets can also include different subsets of genes which may offer more insight into potential mechanisms of toxicity and the ability to have refined predictions of long term toxic consequences such as chronic, irreversible toxicity or carcinogenicity.

Some members of the toxicity predictive genes may also be suitable for prediction of toxicity in other organs or may be preferable for predicting toxicity for wider ranges of timepoints or treatment routes or regimens. As an example of the latter, some of the predictive genes are observed at three different timepoints after treatment. These genes may be useful for prediction in cases where the samples come from treatment protocols that have different measurement timepoints or routes of administration than those employed for the database used in the discovery of the predictive genes disclosed herein or where the toxicokinetics for a particular agent are known or suspected to be different from those in the database.

In one embodiment, the agent is an agent for which no expression profile has been assessed or stored in the database or library. An animal, e.g., rat, is dosed with such an agent and the gene expression profile(s) is the test set for the Predictive Model. The training set which is used in the Predictive Model in this case can be the entire database of sample array data because the test set data is not present in the database. As described in Example 8, the prediction can be made with accuracy without the use of histopathology scores as part of the input into the Predictive Model.

In another embodiment the agent is an agent present in the database but is used at a different dose level or with a different treatment protocol than used in the database. The training set which is used in the Predictive Model in this case can be the entire database of sample array data because the test set data is not present in the database. As described in Example 8, the prediction can be made with accuracy without the use of histopathology scores as part of the input into the Predictive Model.

In another embodiment, the exposure time of the agent is not 6, 24, or 72 hours or repeat dosing protocols are used. In this case, the skilled artisan can use the predictive toxicity genes from surrounding time points to extrapolate the predicted toxicity without undue experimentation. For example, if the individual has been exposed to the agent for, 12 hours, then predictive genes from 6 and 24 hours timepoints are used as guidelines for extrapolating toxicity predictions.

In another embodiment, the toxicity predictive genes and a predictive model can be used to determine the presence or absence of a no-observed toxicity effect level. An agent can be used at different treatment levels and expression profiles obtained for each treatment level. The predictive genes and predictive model can be used to determine which dose levels elicit a response that is predicted to be toxic and which dose levels are not toxic. In contrast to conventional endpoints for determining no-effect levels, the use of expression data, predictive genes and predictive models applies a number of quantitative endpoints and criteria instead of subjective endpoints and criteria. This permits more rigorous and precisely defined determination of no effect levels.

In another embodiment, the toxicity predictive genes can be used to detect toxic effects that may be manifested as long lasting or chronic consequences such as irreversible toxicity or carcinogenesis. The predictive genes and model can be applied to databases where classifications of training and test set samples are made with respect to actual or putative endpoints such as irreversible toxicity or carcinogenicity.

In another embodiment, the predictive genes can be used in a variety of alternative models to predict toxicity. Some of these models do not require the direct use of data in a database but use functions or coefficients derived from the database. In another embodiment, the predictive genes and models may be used to evaluate in vitro systems for their ability to reflect in vivo toxic events and to use such in vitro systems for predicting in vivo toxicity. Expression profiles for predictive genes can be created from candidate in vitro assays using treatments with agents of known in vivo toxicity and for which in vivo data on gene expression are available. The expression data and predictive models of this invention can be used to determine whether the in vitro assay system has predictive gene expression responses that accurately reflect the in vivo situation. Large sets of predictive genes as described in this invention can be tested in such models for their suitability and performance with the candidate in vitro systems. This is a superior and novel tool for evaluating and optimizing in vitro systems for their ability to reflect and accurately predict in vivo responses.

In another embodiment, the predictive genes and models may be used with an in vitro system to accurately predict in vivo toxicity. In vitro systems that have been evaluated and optimized as described in the previous embodiment are treated with test agents and expression profiles are measured for predictive genes. The expression profiles are used in conjunction with a predictive model to predict in vivo toxicity. In this embodiment, there can be considerable reduction in the use of laboratory animals. Additionally the application of this embodiment to in vitro human systems can provide a unique capability to accurately predict human toxic responses without human in vivo exposure or treatment.

In another embodiment, measurement of the expression levels of the proteins encoded by the predictive genes can be used in conjunction with predictive models to predict toxicity. Among the full set of toxicity predictive genes are various genes known to encode cell surface, secreted and/or shed proteins. This enables the development of methods for predicting toxicity using protein biomarkers. For example, as disclosed in Table 33, there are 19 genes in the master predictive set which are known to encode secreted proteins. Thus, in another aspect of the present invention, toxicity predictive assays that detect the expression of one or more of said predictive proteins are developed. Such assays have several advantages, such as:

Ability to use archived tissue specimens such as preserved or embedded tissues that are not suitable for measurement of RNA expression.

Ability to examine predictive protein expression in tissue slides using in situ labeling and microscopic observation. This is useful for detecting predictive toxicity signals occurring in very small sub-populations of cells.

Ability to detect protein markers in specimens that can be readily obtained with little or no invasiveness (e.g., blood, urine, sweat, saliva).

Reduction in animal use in laboratory studies such that no sacrifice of animals necessary to obtain tissue specimens when toxicity prediction can be made with specimens that can be obtained without animal sacrifice or surgery.

Application for human use where tissue specimens cannot be obtained or are only obtained with great difficulty.

In another embodiment, the identified predictive genes can be considered as potential therapeutic targets when the genes are involved in toxic damage or repair responses whose expression or functional modification may attenuate, ameliorate or eliminate disease conditions or adverse symptoms of disease conditions.

In another embodiment the predictive genes can be organized into clusters of genes that exhibit similar patterns of expression by a variety of statistical procedures commonly used to identify such coordinately expression patterns. Common functional properties of these clustered genes can be used to provide insight into the functional relationship of the response of these genes to toxic effects. Common genetic properties of these genes (e.g., common regulatory sequences) may provide insight into function aspects by revealing known or novel similarities in the coding region of the genes. The presence of common known or novel signal transduction systems that regulate expression of the genes can also lead to insight as to the functional properties of the genes. The presence of common known or novel regulatory sequences in the identified predictive genes can also be used to identify toxicity predictive genes that are not present in the current Rat CT array. This can be accomplished by someone skilled in the art who can analyze sequence databases for common regulatory sequences.

In yet another embodiment, the toxicity predictive genes can be used to predict toxicity responses in other species, for example, human, non-human primate, mouse, hamster, guinea pig, hamster, rabbit, cattle, sheep, pig, chicken, and dog. Some members of the toxicity predictive genes may also be more suitable for prediction of toxicity in species other than the species used to derive the database (rat in the case of the examples provided). One method for identification of such genes is that would be available to someone skilled in the art would be to examine DNA sequence databases to determine whether orthologous sequences to the predictive genes exist in the target species and how close the orthologous sequences are to the predictive gene sequences. One of skill in the art can examine the orthologous sequences for similarity in amino acid coding regions and motifs as well as for similarities in regulatory regions and motifs of the gene.

In another embodiment, necrosis predictive genes or gene sequences are used for screening other potential toxicity predictive genes or gene sequences in other species or even within the same species using methods known in the art. See, for example, Sambrook supra. Gene sequences that hybridize under stringent conditions to the toxicity predictive gene sequences disclosed herein may be selected as potential toxicity predictive genes. Additionally, genes which demonstrate significant homology with the toxicity predictive genes disclosed herein (preferably at least about 70%) may be selected as toxicity predictive gene candidates. It is understood that conservative substitutions of amino acids are possible for gene sequences that have some percentage homology with the necrosis predictive gene sequences of this invention. A conservative substitution in a protein is a substitution of one amino acid with an amino acid with similar size and charge. Groups of amino acids known normally to be equivalent are: (a) Ala, Ser, Thr, Pro, and Gly; (b) Asn, Asp, Glu, and Gln; (c) His, Arg, and Lys; (d) Met, Glu, lie, and Val; and (e) Phe, Tyr, and Trp.

It is understood that the predictive toxicity genes can be used as guides to predicting toxicity for agents that have been administered via different routes (intraperitoneal, intravenous, oral, dermal, inhalation, mucosal, etc.) from the routes that were used to generate the database or to identify the toxicity predictive genes. Furthermore, the invention is not intended to be limiting to agents that have been administered at different dosages than the agents that were used to generate the database or to identify the predictive toxicity genes.

Data described in the examples were generated using the microarray technology disclosed in the Examples. However, the invention is not dependent on using this particular platform. Other similar gene expression analysis technologies may be incorporated in the practice of this invention. These can include, but are not limited to, other arrays containing the predictive genes, RT-PCR (e.g., TaqMan®), branched chain technology, RNAse protection or any other method which quantitatively detects the expression of RNA polynucleotides. The invention can be practiced using these other technologies by generating a database of expression measurements for the predictive genes using samples such as those used in the database described in Example 1. This database can then be used in a model such as the K-nearest neighbor model or can be used to develop any of a number of other models.

The following Examples are provided to illustrate but not to limit the invention in any manner.

### EXAMPLE 1

Database of Compounds and Toxicity:

Compounds and treatments list used to construct a database are given in Table 1. This table also provides evaluation of the toxicity observed as necrosis in samples collected 72 hours after treatment.

Database of Animal Experiments:

Sprague Dawley rats Crl:CD from Charles River, Raleigh, N.C. were divided into treated rats that receive a specific concentration of the compound (see Table 1) and. control rats that only received the vehicle in which the compound is mixed (e.g., saline).

At specified timepoints (6 h, 24 h and 72 h) after administration (intraperitoneal route) of the compound, a set number of rats (usually 3 control and 3 treated) were euthanized and tissues collected. Each, rat was heavily sedated with an overdose of CO₂ by inhalation and a maximum amount of blood drawn. Exsanguination of the rat by this drawing of blood kills the rat. The method of collecting the tissues is very important and ensures preserving the quality of the mRNA in the tissues. The body of the rat was then opened up and prosectors rapidly removed the tissues (including) and immediately placed them into liquid nitrogen. The organs/tissues were frozen within 3 minutes of the death of the animal to ensure that mRNA did not degrade. The organs/tissues were then packaged into well-labeled plastic freezer quality bags and stored at −80 degrees until needed for isolation of the mRNA from a portion of the organ/tissue sample.

Isolating DNA/RNA from Animal Tissues or Cells:

Total RNA was isolated from tissue samples using the following materials: Qiagen RNeasy midi kits, 2-mercaptoethanol, liquid N₂, tissue homogenizer, dry ice Samples were kept on ice when specified.

If a tissue needed to be broken, then the tissue sample was placed on a double layer of aluminum foil which was then placed within a weigh boat containing a small amount of liquid nitrogen. The aluminum foil was folded around the tissue and then struck by a small foil-wrapped hammer to administer mechanical stress forces.

About 0.15-0.20 g of tissue was weighed out and placed in a sterile container. To preserve integrity of the RNA, tissues were kept on dry ice when other samples were being weighed. A RLT (Qiagen®) buffer was added to the sample to aid in the homogenization process. The tissue was homogenized using commercially available homogenizer (IKA Ultra Turrax T25 homogenizer) with the 7 mm microfine sawtooth shaft and generator (195 mm long with a processing range of 0.25 ml to 20 ml, item # 372718). After homogenization, samples were stored on ice until samples were homogenized. The homogenized tissue sample was spun to remove nuclei thus reducing DNA contamination. The supernatant of the lysate was then transferred to a clean container containing an equal volume of 70% EtOH in DEPC treated H₂O and mixed. RNA was isolated by putting the supernatant through an RNeasy spin column, washed, and subsequently eluted. Small quantities of remaining DNA were removed by use of DNase enzyme during the RNA isolation procedure following the instructions provided by Qiagen and alternatively by lithium chloride (LiCl) precipitation following the RNA isolation. The isolated RNA pellet was stored in Rnase-free water or in an RNA storage buffer (10 mM sodium citrate), Ambion Cat #7000. The RNA, amount was then quantitated using a spectrophotometer.

Rat 700 CT Chip:

Gene expression data was generated from a microarray chip that has a set of toxicologically relevant rat genes that are used to predict toxicological responses. The rat 700 CT gene array is disclosed in pending U.S. application Ser. Nos. 60/264,933; 60/308,161; and pending application filed on Jan. 29, 2002 (Ser. No. 10/060,893).

Microarray RT Reaction:

Fluorescence-labeled first strand cDNA probe was made from the total RNA or mRNA isolated from s of control and treated rats. This probe was hybridized to microarray slides spotted with DNA specific for toxicologically relevant genes. The materials needed are: total or messenger RNA, primer, Superscript II buffer, dithiothreitol (DTT), nucleotide mix, Cy3 or Cy5 dye, Superscript II (RT), ammonium acetate, 70% EtOH, PCR machine, and ice.

The volume of each sample that would contain 20 μg of total RNA (or 2 μg of mRNA) was calculated. The amount of DEPC water needed to bring the total volume of each RNA sample to 14 μl was also calculated. If RNA was too dilute, the samples were concentrated to a volume of less than 14 μl in a speedvac without heat. The speedvac must be capable of generating a vacuum of 0 Milli-Torr so that samples can freeze dry under these conditions. Sufficient volume of DEPC water was added to bring the total volume of each RNA sample to 14 μl. Each PCR tube was labeled with the name of the sample or control reaction. The appropriate volume of DEPC water and 8 μl of anchored oligo dT mix (stored at −20° C.) was added to each tube.

Then the appropriate volume of each RNA sample was added to the labeled PCR tube. The samples were mixed by pipeting. The tubes were kept on ice until samples are ready for the next step. It is preferable for the tubes to kept on ice until the next step is ready to proceed. The samples were incubated in a PCR machine for 10 minutes at 70° C. followed by 4°C. incubation period until the sample tubes were ready to be retrieved. The sample tubes were left at 4° C. for at least 2 minutes.

The Cy dyes are light sensitive, so any solutions or samples containing Cy-dyes should be kept out of light as much as possible (e.g., cover with foil) after this point in the process. Sufficient amounts of Cy3 and Cy5 reverse transcription mix were prepared for one to two more reactions than would actually be run by scaling up the following:

For Labeling with Cy3

8 ul 5× First Strand Buffer for Superscript II, 4 ul 0.1 M DTT, 2 ul Nucleotide Mix, 2 ul of 1:8 dilution of Cy3 (e.g., 0.125 mM cy3dCTP) and 2 ul Superscript II

For labeling with Cy5

8 ul 5× First Strand Buffer for Superscript II, 4 ul 0.1M DTT, 2 ul Nucleotide Mix, 2 ul of 1:10 dilution of Cy5 (e.g., 0.1 mM Cy5dCTP) and 2 ul Superscript II

About 18 μl of the pink Cy3 mix was added to each treated sample and 18 μl of the blue Cy5 mix was added to each control sample. Each sample was mixed by pipeting. The samples were placed in a DNA engine (PTC-200 Petier Thermal Cycler, M. J. Research) for 2 hours at 45° C. followed by 4° C. until the sample tubes were ready to be retrieved.

In addition to the desired cDNA product, the RT reaction contained impurities that must be removed. These impurities included excess primers, nucleotides, and dyes. The primary method of removing the impurities was by following the instructions in the QIAquick PCR purification kit (Qiagen cat#120016).

Alternatively, the RT reactions were cleaned of impurities by ethanol precipitation and resin bead binding. The samples from DNA engine were transferred to Eppendorf tubes containing 600 μl of ethanol precipitation mixture and placed in −80° C. freezer for at least 20-30 minutes. These samples were centrifuged for 15 minutes at 20800×g (14000 rpm in Eppendorf model 5417C) and carefully the supernatant was decanted. A visible pellet was seen (pink/red for Cy3, blue for Cy5). Ice cold 70% EtOH (about 1 ml per tube) was used to wash the tubes and the tubes were subsequently inverted to clean tube and pellet. The tubes were centrifuged for 10 minutes at 20800×g (14000 rpm in Eppendorf model 5417C), then the supernatant was carefully decanted. The tubes were air dried for about 5 to 10 minutes, protected from light. When the pellets were dried, they were resuspended in 80 ul nanopure water. The cDNA/mRNA hybrid was denatured by heating for 5 minutes at 95° C. in a heat block and flash spun. Then the lid of a “Millipore MAHV N45” 96 well plate was labeled with the appropriate sample numbers. A blue gasket and waste plate (v-bottom 96 well) was attached. About 160 μl of Wizard DNA Binding Resin (Promega cat#A1151) was added to each well of the filter plate that was used. Probes were added to the appropriate wells (80 μl cDNA samples) containing the Binding Resin. The reaction is mixed by pipeting up and down ˜10 times. The plates were centrifuged at 2500 rpm for 5 minutes (Beckman GS-6 or equivalent) and then the filtrate was decanted. About 200 μl of 80% isopropanol was added, the plates were spun for 5 minutes at 2500 rpm, and the filtrate was discarded. Then the 80% isopropanol wash and spin step was repeated. The filter plate was placed on a clean collection plate (v-bottom 96 well) and 80 μl of Nanopure water, pH 8.0-8.5 was added. The pH was adjusted with NaOH. The filter plate was secured to the collection plate and after 5 minutes was centrifuged for 7 minutes at 2500 rpm.

Purification of Cy-Dye Labeled cDNA:

To purify fluorescence-labeled first strand cDNA probes, the following materials were used: Millipore MAHV N45 96 well plate, v-bottom 96 well plate (Costar), Wizard DNA binding Resin, wide orifice pipette tips for 200 to 300 μl volumes, isopropanol, nanopure water. It is highly preferable to keep the plates aligned at times during centrifugation. Misaligned plates lead to sample cross contamination and/or sample loss. It is also important that plate carriers are seated properly in the centrifuge rotor.

The lid of a “Millipore MAHV N45” 96 well plate was labeled with the appropriate sample numbers. A blue gasket and waste plate (v-bottom 96 well) was attached. Wizard DNA Binding Resin (Promega cat#A1151) was shaken immediately prior to use for thorough resuspension. About 160 μl of Wizard DNA Binding Resin was added to each well of the filter plate that was used. If this was done with a multi-channel pipette, wide orifice pipette tips would have been used to prevent clogging. It is highly preferable not to touch or puncture the membrane of the filter plate with a pipette tip. Probes were added to the appropriate wells (80 μl cDNA samples) containing the Binding Resin. The reaction is mixed by pipeting up and. down ˜10 times. It is preferable to use regular, unfiltered pipette tips for this step. The plates were centrifuged at 2500 rpm for 5 minutes (Beckman GS-6 or equivalent) and then the filtrate was decanted. About 200 μl of 80% isopropanol was added, the plates were spun for 5 minutes at 2500 rpm, and the filtrate was discarded. Then the 80% isopropanol wash and spin step was repeated. The filter plate was placed on a clean collection plate (v-bottom 96 well) and 80 μl of Nanopure water, pH 8.0-8.5 was added. The pH was adjusted with NaOH. The filter plate was secured to the collection plate with tape to ensure that the plate did not slide during the final spin. The plate sat for 5 minutes and was centrifuged for 7 minutes at 2500 rpm. Replicates of samples should be pooled.

Dry-Down Process:

Concentration of the cDNA probes is preferable so that they can be resuspended in hybridization buffer at the appropriate volume. The volume of the control cDNA (Cy-5) was measured and divided by the number of samples to determine the appropriate amount to add to each test cDNA (Cy-3). Eppendorf tubes were labeled for each test sample and the appropriate amount of control cDNA was allocated into each tube. The test samples (Cy-3) were added to the appropriate tubes. These tubes were placed in a speed-vac to dry down, with foil covering any windows on the speed vac. At this point, heat (45° C.) may be used to expedite the drying process. Samples may be saved in dried form at −20° C. for up to 14 days.

Microarray Hybridization:

To hybridize labeled cDNA probes to single stranded, covalently bound DNA target genes on glass slide microarrays, the following material were used: formamide, SSC, SDS, 2 μm syringe filter, salmon sperm DNA (Sigma, cat # D-7656), human Cot-1 DNA (Life Technologies, cat # 15279-011), poly A (40 mer: Life Technologies, custom synthesized), yeast tRNA (Life Technologies, cat # 15401-04), hybridization chambers, incubator, coverslips, parafilm, heat blocks. It is preferable that the array is covered to ensure proper hybridization.

About 30 μl of hybridization buffer was prepared per cDNA sample (control rat cDNA plus treated rat cDNA). Slightly more than is what is needed should be made since about 100 μl of the total volume made for hybridizations can be lost during filtration.

| Hybridization Buffer: | for 100 μl: |
|---|---|
| 50% Formamide | 50 μl formamide |
| 5× SSC | 25 μl 20× SSC |
| 0.1% SDS | 25 μl 0.4% SDS |

The solution was filtered through 0.2 μm syringe filter, then the volume was measured. About 1 μl of salmon sperm DNA (10 mg/ml) was added per 100 μl of buffer.

Alternatively, the hybridization buffer was made up as:

| Hybridization Buffer: | for 101 μl: |
|---|---|
| 50% Formamide | 50 μl formamide |
| 10× SSC | 50 μl 20× SSC |
| 0.2% SDS | 1 μl 20% SDS |

The solution was filtered through 0.2 μm syringe filter, then the volume was measured. One microliter of salmon sperm DNA (9.7 mg/ml), 0.5 μl Human Cot-1 DNA (5 μg/μl), 0.5 μl poly A (5 μg/μl), 0.25 μl Yeast tRNA (10 μg/μl) was added per 100 μl of buffer. The hybridization buffers were compared in validation studies and there was no change in differential gene expression data between the two buffers.

Materials used for hybridization were: 2 Eppendorf tube racks, hybridization chambers (2 arrays per chamber), slides, coverslips, and parafilm. About 30 μl of nanopure water was added to each hybridization chamber. Slides and coverslips were cleaned using N₂ stream. About 30 μl of hybridization buffer was added to dried probe and vortexed gently for 5 seconds. The probe remained in the dark for 10-15 minutes at room temperature and then was gently vortexed for several seconds and then was flash spun in the microfuge. The probes were boiled or placed in a 95° C. heat block for 5 minutes and centrifuged for 3 min at 20800×g (14000 rpm, Eppendorf model 5417C). Probes were placed in 70° C. heat block. Each probe remained in this heat block until it was ready for hybridization.

About 25 μl was pipeted onto a coverslip. It is highly preferable to avoid the material at the bottom of the tube and to avoid generating air bubbles. This may mean leaving about 1 μl remaining in the pipette tip. The slide was gently lowered, face side down, onto the sample so that the coverslip covered that portion of the slide containing the array. Slides were placed in a hybridization chamber (2 per chamber). The lid of the chamber was wrapped with parafilm and the slides were placed in a 42° C. humidity chamber in a 42° C. incubator. It is preferable to not let probes or slides sit at room temperature for long periods. The slides were incubated for 18-24 hours.

Post-Hybridization Washing:

To obtain only single stranded cDNA probes tightly bound to the sense strand of target cDNA on the array, non-specifically bound cDNA probe should be removed from the array. Removal of non-specifically bound cDNA probe was accomplished by washing the array and using the following materials: slide holder, glass washing dish, SSC, SDS, and nanopure water. Six glass buffer chambers and glass slide holders were set up with 2× SSC buffer heated to 30-34° C. and used to fill up glass dish to ¾^{th} of volume or enough to submerge the microarrays. The slides were placed in 2× SSC buffer for 2 to 4 minutes while the cover slips fall off. The slides were then moved to 2× SSC, 0.1% SDS and soaked for 5 minutes. The slides were transferred into 0.1× SSC and 0.1% SDS for 5 minutes. Then the slides are transferred to 0.1× SSC for 5 minutes. The slides, still in the slide carrier, were transferred into nanopure water (18 megaohms) for 1 second. To dry the slides, the stainless steel slide carriers were placed on micro-carrier plates and spun in a centrifuge (Beckman GS-6 or equivalent) for 5 minutes at 1000 rpm.

Scanning Slides:

The washed and dried hybridized slides were scanned on Axon Instruments Inc. GenePix 4000A MicroArray Scanner and the fluorescent readings from this scanner converted into quantitation files (.gpr) on a computer using PenePix software.

Array Data, Normalization and Transformation:

GeneSpring™ software (Version 4.1, Silicon Genetics) was used for statistical analyses including identification of genes expressions correlating with histopathology scores, K-means and tree cluster analysis, and predictive modeling using the K-means nearest neighbor (Predict Parameter Values tool).

Microarray data were loaded into GeneSpring™ software for analysis as GenePix files as above. Specific data loaded into GeneSpring™ software included gene name, GenBank ID control channel mean fluorescence and signal channel mean fluorescence. Expression ratio data (ratio of signal to control fluorescence) were normalized using the 50^{th} percentile of the distribution of genes and control channel. Ratio data were excluded from analysis if the control channel value was <0. For analysis of correlations and predictive values gene expression ratios were transformed as the log of the ratio.

Correlation with Histopathology Scores:

Histopathology scores for each animal (assigned on a compound-dose basis as indicated in Table 1) were entered with gene expression data by using the GeneSpring™ ‘Drawn Gene’ function. Correlations between the histopathology scores and gene expression were conducted with the distance measures listed below:

| | |
|---|---|
| standard | positive and negative correlation |
| smooth | positive and negative correlation |
| change | positive correlation |
| upregulated | positive correlation |
| Pearson | positive and negative correlation |
| Spearman | positive and negative correlation |
| distance | positive correlation |

These correlation or similarity measures are standard statistical correlation measures that are described in the GeneSpring Advanced Analysis Techniques Manual (Release Data Mar. 13, 2001, Silicon Genetics). Where both positive and negative correlations were obtained combined positive and negative correlating gene lists were also created.

Class Prediction:

The Predict Parameter Values tool in GeneSpring™ software was used for toxicity class prediction. The following is a summary of the procedure used in the GeneSpring predictive software. This is described in GeneSpring Advanced Analysis Techniques Manual (Release Data Mar. 13, 2001, Silicon Genetics) with additional information supplied by Silicon Genetics and a statistical expert. The prediction tool relies on standard statistical procedures that can be implemented in a variety of statistical software packages.

Gene Selection:

Genes to be used for prediction are picked through variable selection. This entails taking a single gene and a single class (e.g., toxicity) and creating a contingency table. In the table below, columns 1 through N of the table each represent one possible cutoff point based on the gene expression level (ratio of signal/control) for that class. The number of possible cutoffs is less than or equal to the total number of samples for the class (e.g., A). It is possibly less than the total number, since there may be ties in gene expression level. Hence, N, M, and X may or may not be distinct. In the example, an n-class problem is illustrated, where x and y entries are the class counts at that gene expression cutoff level, for that specific gene and class, either above (“a”) or below (“b”) the cutoff. “Class1” is the set of all samples (above or below) the cutoff for Class1, and “!Class1” are all those not in Class1 (above or below) the cutoff, and similarly for the other classes. The class totals in the training set are the total class marginals used to compute Fisher's exact test.

For a specific gene, and for each class, the best p-value as calculated by Fisher's Exact Test for independence between one of the pair of columns (e.g., 1a and 1b) and the actual class totals (e.g., A) is used to score the gene (−In(p)=the score) for that class. Thus, there are N (or, M, Q etc.) contingency tables, where the best score of the N tables is used for that class and gene. If there is a wide disparity between the above and below counts in either the a or b column (this is a two-sided Fisher's Exact Test), the smaller the p-value and the higher the score.

The genes per class are rank ordered by the most discriminating (highest) score. The predictivity list is composed of the most discriminating genes per class. Namely, genes are combined that best discriminate class 1 with those that best discriminate class 2 and so on. The genes are selected in rotation of the highest score per class. Duplicate genes are ignored in the rotation and not added to the list, the gene with the next highest score is taken.

The training samples now have only the gene list garnered from the above procedure. As an example, where once the training samples may have had an initial list of 200 genes per sample, they now have only a subset composed of the gene list, say, 50 (the number of predictivity genes specified) that are selected from the initial list by the gene selections procedure. Thus, each sample is a vector of 50 normalized expression ratios. Since the selection of genes is done in rotation, the list contains 25 genes for one class, and 25 for the other class. The matrix below illustrates the basic features of this gene selection process.

| | 1a | 1b | | Na | Na | Actual |
|---|---|---|---|---|---|---|
| Gene 1 | Expression | Expression | | Expression | Expression | Class Totals |
| Class | above | below | | above | below | (Marginals) |
| Class1 | x1.1a | x1.1b | . . . | x1.Na | x1.Nb | A |
| !Class1 | y1.1a | y1.1b | . . . | y1.Na | y1.Nb | B |
| Gene 1 | 1 | 2 | . . . | M | | |
| Class2 | x1.2a | x1.2b | . . . | x1.Ma | | C |
| !Class2 | y1.2a | y1.2b | . . . | y1.Ma | | D |
| . | . | . | . | . | | . |
| . | . | . | . | . | | . |
| . | . | . | . | . | | . |
| Gene 1 | 1 | 2 | . . . | Qa | Qb | |
| Classn | x1.na | x1.nb | . . . | x1.Qa | x1.Qb | X |
| !Classn | y1.na | y1.nb | . . . | y1.Qa | y1.Qb | Y |

Classifying the Test Samples:

After the genes to be used in the training set have been selected, the test set is classified based on the k-nearest neighbor (knn) voting procedure. Using just those genes in the gene list, for each sample in the test set of samples, the k nearest neighbors in the training set are found with the Euclidean distance. The class in which each of the k nearest neighbors is determined, and the test set sample is assigned to the class with the largest representation in the k nearest neighbors after adjusting for the proportion of classes in the training set.

For example, in a two-class problem, let there be 30 samples of class 1 and 60 samples of class 2 in the training set. With k=9 say it can be determined that 7 of the nearest neighbors to a sample from the testing set are in class 1. The sample can then be classified as being a member of class 1. If another sample from the test set has a total of 4 nearest neighbors in class 1, after adjusting for the proportion, this sample would be assigned to class 1 rather than class 2, even though the majority vote suggests assignation to class 2.

Decision Threshold:

The decision threshold is a mechanism to help clearly define the class into which the sample will fall, and can be set to reject classification if the voting is very close or tied. (Thus, k can be even for two-class problems without worrying about the tie problem.) A p-value is calculated for the proportion of neighbors in each class against the proportions found in the training set, again using Fisher's exact test, but now a one-sided test.

For example, let k=11, if the proportion of neighbors of class 1 in the test set is 6/11, and the proportion of class 1 in a 100 sample training set is 0.4, the p-value calculated is 0.29 (half the two-sided test). If the proportion in the training set is 0.1, the p-value is 0.004. The smaller the p-value the greater the likelihood that the sample from the testing set belongs to that class.

A p-value ratio (P-value) is set as a way of setting the level of confidence in individual sample predictions based on the ratio of p-values for the best class (lowest p-value) versus the second best class (second lowest p-value). For example, if the P-value is set at 0.5 and the ratio of p-values for a particular sample is 0.6, then the predictive model will not make a call for that sample.

Training and Test Data Sets:

Data were each separated into 5 training and test sets by randomly distributing the compounds into the sets. This was accomplished by assigning random numbers to lists of compounds that are negative and positive for histopathology, sorting by random number, and then dividing the sorted lists into a specific number of training and test sets. The training and test set assignments are presented in Table 2.

Toxicology Classification:

Toxicity classifications were entered for training and test set as a parameter column. Toxicity, as defined by observation of necrosis in the at 72 hours after treatment, was entered as a “yes” or “no” for each animal in a compound-dose group. Additionally, a parameter column for random histopathology classification was designated. This was done by randomly assigning the same number of “yes” and “no” calls to the individual animals.

Prediction Output and Initial Data Processing:

The “Predict Parameter Value” tool of GeneSpring was used with each of the training and test sets to generate predictions of histopathology classifications of the test sets. Unless otherwise specified a nearest neighbor setting of 10 (default) and P-value ratio cutoff of 0.5 was used. The number of genes used to predict was varied with standard numbers of 50, 40, 30, 20, 10, 5, 2 and 1 genes used. For each number of genes the numbers of correct calls, incorrect calls and non-calls were recorded. Non-calls are cases where no prediction was made because the P-value ratio exceeded the specified P-value ratio cutoff. Calculations were made for overall percent correct calls (number of correct classifications/number or samples), percent correct calls of called samples (number of correct classifications/number of samples with calls) and percent of called samples (samples with calls/number of samples).

For each input list and optimal number of predictive genes (lowest number of genes giving a maximum overall percent of correct calls) additional information was recorded that included the list of specific genes in the optimum predictive set.

Results:

Expression array data were examined for the existence of genes whose expression correlated with histopathology scores. Table 1 presents a list of the compounds and dose levels along with the histopathology classification and histopathology severity scores used for this analysis. For each distance measure the probability was adjusted in increments of 0.05 until at least 50 correlating genes were obtained. Lists of correlating genes were obtained using the distance measures described in Materials and Methods. Example sets of correlating genes are provided in Tables 3 and 4.

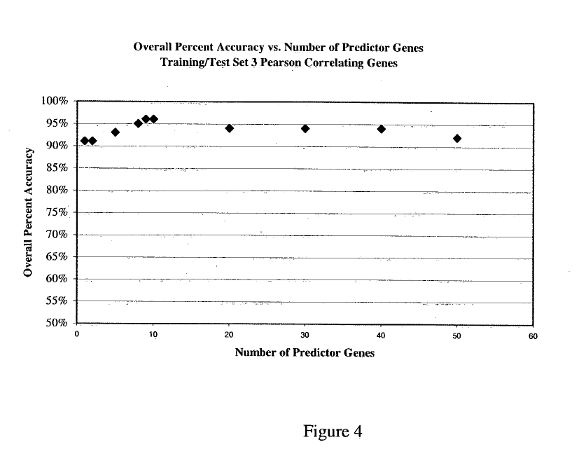

The correlating gene lists as well as the entire array gene list were provided as input lists to the GeneSpring Predict Parameter value tool (described in Materials and Methods) that employs a K-means nearest neighbor (knn) predictive model. These lists as well as the entire array gene list were used for each of the five training and test sets defined in Materials and Methods to generate predictions of histopathology classifications of the test sets. Input genes for the Predict Parameter Value feature included all 700 genes in the GenePix file (the rat CT Array) which were disclosed in a currently pending application (Ser. No. 10/060,893) filed on Jan. 29, 2002, as well as smaller lists of genes whose expressions correlated with histopathology by the correlation measures described previously. The number of genes used to predict are varied with standard numbers of 50, 40, 30, 20, 10, 5, 2 and 1 genes used. The specified number of predictive genes was varied to obtain an optimum number of predictive genes. FIG. 4 presents a typical profile for obtaining an optimum gene list.

After this was done for 5 training and test sets, all gene lists were then merged to create one aggregate list of predictive genes. Each gene on this aggregate list has predictive value for at least one of the training and test sets because it was observed to contribute to an optimum predictivity for a specific training/test set. The aggregate list was subdivided into smaller lists of genes based on the number of times a gene was predictive for an individual training or test set. For example, if 5 training and test sets were used, genes that were predictive in 5 training and test sets were designated as Combo (combination) 5. Genes that were predictive in only 4 of 5 training and test sets were designated as Combo 4, etc. A list of predictive genes organized by their occurrence in the separate training and test sets is presented in Table 5.

### EXAMPLE 2

Materials and Methods:

The database used was as described in Example 1.

Array Data, Normalization and Transformation:

Array data, normalization procedures and transformations used in these analyses are as described in Example 1. Table 32 presents 24 hour gene expression data for the predictive genes. These data can be used with a k nearest neighbor prediction model (as available in GeneSpring or other statistical software packages) to make predictions as described in this example.

Class Prediction:

The Predict Parameter Values tool in GeneSpring™ software was used for toxicity class prediction. A description of this tool and the statistical procedures used is provided in Example 1.

Training and Test Data Sets:

The training and test data sets used are those described in Table 2 of Example 1.

Toxicology Classification:

Toxicity classifications used are described in Table 1 of Example 1. In this analysis randomized classifications (same number of “yes” and “no” classifications distributed randomly among the samples) were also used.

Prediction Output and Initial Data Processing:

For each predicting gene list used for evaluation a table of data generated by the Predict Parameter Values tool in GeneSpring™ software was saved which provided. for each sample in the test set the actual call (“yes” or “no” for toxicity), the predicted call (“yes”, “no” or no call for toxicity) and the P-value cutoff ratio. This set of data was used to calculate predictive performance measures provided below.

Prediction Measures:

Measures of prediction used for these analyses are generally accepted prediction measures for information about actual and predicted classifications done by a classification system (Modern Applied Statistics with S-Plus, W. N. and B. D. Ripley, Springer, 1994, 3^{rd} edition.; Proc. 14^{th} International Conference on Machine Learning, Miroslav Kubat, Stan Matwin, 1997). Results from predictions of a two class case can be described as a two-class matrix:

Predicted

| Actual | Negative | Positive |
|---|---|---|
| Negative | a | b |
| Positive | c | d |

Standard terms used for prediction are:

Accuracy is the proportion of total number of predictions that are correct=a+d/a+b+c+c

False positive rate is the proportion of negative cases that are incorrectly classified as positive=b/a+b

False negative rate is the proportion of positive cases that are incorrectly classified as negative=c/c+d

Geometric-mean is the performance measure that takes into account proportion of positive and negative cases (Kubat et al., ibid)=the square root of TP*TN where TP=true positive rate (d/c+d) and TN=true negative rate (a/a+b). In these analyses cases where no prediction was made because the p-value ratio exceeded the cutoff-value (generally 0.5) the non-call was considered to be incorrect.

Random Selected Gene Sets:

Subsets of randomly selected genes were prepared from the predictive gene sets to test whether such subsets would have predictive value. Assignments of genes to these subsets are presented in Tables 6-7. Genes were also randomly selected from the list of all genes excluding the 142 twenty-four hour predictive genes (also known as non-predictive genes) by assigning a random number to each gene, sorting by the random number and selecting the appropriate number of sorted genes. Assignments of genes to these subsets are presented in Table 8.

Results:

Prediction results for 24 hour expression data using genes identified as predictive are presented in Table 9. These data indicate a very high accuracy in predicting toxicity. Mean accuracy exceeded 0.92 (92% accuracy) for the entire predictive gene list (Combo All) and all the Combo gene lists. Because these predictions were conducted with multiple training/test set combinations it is possible to obtain an indication of the variability in prediction rates and robustness of the prediction capabilities of these gene sets. For the Combo All and other Combo lists there was very good predictivity for all training/test sets of data with over 0.75 (75%) accuracy as a minimum value for any one training and test set and most lists giving over 0.8 (80%) minimum accuracy. False positive and false negative prediction rates were generally low with means generally less than 0.15 (15%) for all Combo lists. The geometric mean was used as an indication of predictive performance that includes consideration of the proportion of positive and negative classifications. All gene sets gave geometric mean measures >0.8 (80%) and four gene sets (Combo All, Combo 5, Combo 3 and Combo 2 gene lists) had mean measures >0.9.

As described in Materials and Methods in those cases where no prediction was made because the p-value ratio exceeded the cutoff-value (generally 0.5) the non-call was considered to be incorrect.

Prediction results for 24 hour expression data using genes identified as predictive and the predicting unit of compound-dose are presented in Table 10. This prediction unit is probably the most relevant for toxicology prediction. The performance of the genes in predicting compound-dose toxicity is even better than predictions on an individual animal basis. These data indicate a very high accuracy in predicting toxicity. Mean accuracy exceeded 0.9 (90% accuracy) for the entire predictive gene list (Combo All) and all of the Combo gene lists. Accuracy and was comparable for all the Combo lists. Variability in accuracy was low for most of the gene lists with >0.8 (80%) minimum accuracy for any single training and test set observed for the Combo All and Combo 5, 4, 2 and 1 gene lists. Particularly noteworthy on the compound-dose level prediction is the low false-negative rate and false positive rates observed for all of the Combo sets. The geometric mean measure of predictive performance also indicated excellent predictive properties for all gene sets.

One noteworthy feature of the predictive capability is the ability to distinguish between effects of a compound at different dose levels. Four compounds (ANIT, APAP, LPS and TET) produced toxicity at the high dose but not at the low dose. The predictive gene sets were usually accurate in predicting toxicity at the high dose and predicting no toxicity at the low dose.

Prediction results for 24 hour expression data using genes identified as predictive and the predicting unit is compound are presented in Table 11.

Predictive performance on a compound basis with accuracies and geometric mean measures being at or above 0.9 (90%) and very low false positive and false negative error rates. Table 12, 13, and 14 show the level of predictive accuracy of individual genes of Combos 5, 4, and 3, respectively, for 24 hour data.

The tables show that overall, individual genes of the Combo groups did not perform as well as the combination as a whole, as the average predictive accuracy of individual genes versus the entire combo set was 82.6% vs. 89.6% for Combo 5, 80.8% vs. 85.7% for Combo 4, and 69.8% vs. 86.5% for Combo 3. The table also shows that while many of the individual genes of the Combo groups gave a good level of predictive accuracy (as high as 89.2% for individual genes of Combo 5, 90.6% for Combo 4, and 82.1% for Combo 3), the predictive accuracy of individual genes rarely exceeded the predictive accuracy of the whole combination.

In order to assess the performance of subsets of genes, predictive performance was evaluated for subsets of genes randomly selected from the total combined predictive list (Combo All) and the top Combo sets (as defined in Materials and Methods). Prediction results for 24 hour expression data using randomly selected subsets of genes are presented in Table 15. These data clearly indicate that smaller subsets of the Combo gene lists have predictive power.

Table 16 compares prediction accuracy for correct classification of toxicity and for the same proportion of positive and negative toxicity calls randomly assigned to the samples (random classification). For each gene set or subset predictions were made using the same five training/test sets as for the other prediction analyses. Additionally, sets of genes were randomly chosen from the array which were not identified on the list of 142 predictive genes at 24 hour (Example 1, Table 5).

It is clear from these data that the predictions with accurate classification are much better than predictions with randomized classification. This means that the predictive results are not simply due to chance and large data sets but are due to significant, meaningful predictive association between the gene expression of the predictive genes and the toxicity. The accuracy numbers for the gene sets selected from a list of all genes on the array minus the predictive genes are much lower than the Combo predictive lists and the random subsets of these predictive lists. This also verifies the predictive power of the identified predictive genes. The fact that the predictive numbers from these subsets are somewhat higher for accurate than random classification is likely due to some residual predictivity in these genes that is not very substantial.

### EXAMPLE 3

Materials and Methods:

Compounds and treatments list used to construct the database are given in Table 1 of Example 1. This table also provides the evaluation of the toxicity observed as hepatocellular necrosis in samples collected 72 hours after treatment. A database is described in detail in Example 1. This Example analyzes expression data from samples collected 6 hours after treatment.

Array Data, Normalization and Transformation:

Array data, normalization and transformation procedures used were as described in Example 1.

Correlation with Histopathology Scores:

Procedures and methods for obtaining gene lists correlating with histopathology scores were as described in Example 1 (Table 1).

Class Prediction:

The Predict Parameter Values tool in GeneSpring™ software used for toxicity class prediction is described in detail in Material and Methods of Example 1.

Training and Test Data Sets:

Data were each separated into 5 training and test sets by randomly distributing the compounds into the sets. This was accomplished by assigning random numbers to lists of compounds that are negative and positive for histopathology, sorting by random number, and then dividing the sorted lists into a specific number of training and test sets. The training and test set assignments are presented in the following Table 17.

Toxicity Classification:

Toxicity classifications were entered for training and test set as a parameter column. Toxicity, as defined by observation of hepatocellular necrosis in the at 72 hours after treatment, was entered as a “yes” or “no” for each animal in a compound-dose group. Additionally, a parameter column for random histopathology classification was designated. This was done by randomly assigning the of “yes” and “no” calls to the individual animals such that the total number of “yes” and “no” calls were the same as the correctly assigned classification.

Prediction Output and Initial Data Processing:

The “Predict Parameter Value” tool of GeneSpring was used with each of the training and test sets to generate predictions of histopathology classifications of the test sets. Unless otherwise specified a nearest neighbor setting of 10 (default) and P-value ratio cutoff of 0.5 was used. The number of genes used to predict was varied with standard numbers of 50, 40, 30, 20, 10, 5, 2 and 1 genes used. For each number of genes the numbers of correct calls, incorrect calls and non-calls were recorded. Non-calls are cases where no prediction was made because the P-value ratio exceeded the specified P-value ratio cutoff. Calculations were made for overall percent correct calls (number of correct classifications/number or samples), percent correct calls of called samples (number of correct calls/number of samples with calls) and percent of called samples (samples with calls/number of samples).

For each input list and optimal number of predictive genes (lowest number of genes giving a maximum overall percent of correct calls) additional information was recorded that included the list of specific genes in the optimum predictive set.

Results:

Expression array data were examined for the existence of genes whose expression correlated with histopathology scores. Table 1 in Materials and Methods of Example 1 presents a list of the compounds and dose levels along with the histopathology classification and histopathology severity scores used for this analysis. For each distance measure the probability was adjusted in increments of 0.05 until at least 50 correlating genes were obtained. Lists of correlating genes were obtained using the distance measures described in Materials and Methods. Example sets of correlating genes are provided in Tables 18-19.

The correlating gene lists as well as the entire array gene list were provided as input lists to the GeneSpring Predict Parameter value tool (described in Materials and Methods) that employs a K-means nearest neighbor (knn) predictive model.

These lists as well as the entire array gene list were used for each of the six training and test sets defined in Materials and Methods o generate predictions of histopathology classifications of the test sets. Input genes for the Predict Parameter Value feature included all 700 genes in the GenePix file (the Rat CT Array) as well as smaller lists of genes whose expressions correlated with histopathology by the correlation measures described previously. The number of genes used to predict are varied with standard numbers of 50, 40, 30, 20, 10, 5, 2 and 1 genes used. The specified number of predictive genes was varied to obtain an optimum number of predictive genes.

After this was done for 5 training and test sets, gene lists were then merged to create one aggregate list of predictive genes. Each gene on this aggregate list has predictive value for at least one of the training and test sets because it was observed to contribute to an optimum predictivity for a specific training/test set. The aggregate list was subdivided into smaller lists of genes based on the number of times a gene was predictive for an individual training or test set. For example, if 5 training and test sets were used, genes that were predictive in 5 training and test sets were designated as Combo (combination) 5. Genes that were predictive in only 4 of 5 training and test sets were designated as Combo 4, etc.

A list of predictive genes organized by their occurrence in the separate training and test sets is presented in Table 20.

### EXAMPLE 4

Materials and Methods:

The database used was as described in Example 1.

Array Data, Normalization and Transformation:

Array data, normalization procedures and transformations used in these analyses are as described in Example 1. Table 34 lists 6 hour gene expression data for the predictive genes. These data can be used with a k-means nearest neighbor prediction model (as available in GeneSpring or other statistical software packages) to make predictions as described in this example

Class Prediction:

The Predict Parameter Values tool in GeneSpring™ software was used for toxicity class prediction. A description of this tool and the statistical procedures used is provided in Example 1.

Training and Test Data Sets:

The training and test data sets used are those described in Table 17 of Example 3.

Toxicology Classification:

Toxicology classifications used are described in Table 1 of Example 1. In this analysis randomized classifications (same number of “yes” and “no” classifications distributed randomly among the samples) were used.

Prediction Output and Initial Data Processing:

For each gene list prediction used for evaluation a table of data generated by the Predict Parameter Values tool in GeneSpring™ software was saved which provided for each sample in the test set the actual call (“yes” or “no” for toxicity), the predicted call (“yes”, “no” or no call for toxicity) and the P-value cutoff ratio. This set of data was used to calculate predictive performance measures provided below.

Prediction Measures:

Measures of prediction used for these analyses are generally accepted prediction measures for information about actual and predicted classifications done by a classification system (Modern Applied Statistics with S-Plus, W. N. Venables and B. D. Ripley, Springer, 1994, 3^{rd} edition; Proc. 14^{th} International Conference on Machine Learnig, Miroslav Kubat, Stan Matwin, 1997). Results from predictions of a two class case can be described as a two-class matrix:

Predicted

| Actual | Negative | Positive |
|---|---|---|
| Negative | a | b |
| Positive | c | d |

Standard terms used for prediction are:

Accuracy is the proportion of total number of predictions that are correct=a+d/a+b+c+c

False positive rate is the proportion of negative cases that are incorrectly classified as positive=b/a+b

False negative rate is the proportion of positive cases that are incorrectly classified as negative=c/c+d

Geometric-mean is the performance measure that takes into account proportion of positive and negative cases (Kubat et al., ibid)=the square root of TP*TN where TP=true positive rate (d/c+d) and TN=true negative rate (a/a+b). In these analyses cases where no prediction was made because the p-value ratio exceeded the cutoff-value (generally 0.5) the non-call was considered to be incorrect.

Results:

Prediction results for 6 hour expression data using genes identified as predictive are presented in Table 21. These data indicate accuracy in predicting toxicity with 6 hr expression data. Mean accuracy exceeded 0.7 (70% accuracy) for the entire predictive gene list (Combo All) and 0.6 (60%) for the Combo gene lists. Mean false positive and false negative values were in the range of 0.3-0.4 for the best predicting gene sets and the geometric mean measures were higher than 0.6 except for the Combo 1 gene set. Comparison of predictive performance for correct and random classification is given in Table 22.

It is clear from these data that the predictions with accurate classification are much better than predictions with randomized classification. This means that the predictive results are not simply due to chance and large data sets but are due to significant, meaningful predictive association between the gene expression of the predictive genes and the toxicity.

### EXAMPLE 5

Database-Compounds and Toxicity:

Compounds and treatments list used to construct the database are given in Table 1 of Example 1. This table also provides the evaluation of the toxicity observed as hepatocellular necrosis in samples collected 72 hours after treatment. The Phase-1 Database is described in detail in Example 1. This Example analyzes expression data from samples collected 72 hours after treatment.

Array Data, Normalization and Transformation:

Array data, normalization and transformation procedures used were as described in Example 1.

Correlation with Histopathology Scores:

Procedures and methods for obtaining gene lists correlating with histopathology scores were as described in Example 1 with scores as in Example 1, Table 1.

Class Prediction:

The Predict Parameter Values tool in GeneSpring™ software used for toxicity class prediction is described in detail in Material and Methods of Example 1.

Training and Test Data Sets:

Data were each separated into 5 training and test sets by randomly distributing the compounds into the sets. This was accomplished by assigning random numbers to lists of compounds that are negative and positive for histopathology, sorting by random number, and then dividing the sorted lists into a specific number of training and test sets. The training and test set assignments are presented in the Table 23.

Toxicology Classification:

Toxicity classifications were entered for training and test set as a parameter column. Toxicity, as defined by observation of hepatocellular necrosis in the at 72 hours after treatment, was entered as a “yes” or “no” for each animal in a compound-dose group. Additionally, a parameter column for random histopathology classification was designated. This was done by randomly assigning the same number of “yes” and “no” calls to the individual animals.

Prediction Output and Initial Data Processing:

The ‘Predict Parameter Value’ tool of GeneSpring was used with each of the training and test sets to generate predictions of histopathology classifications of the test sets. Unless otherwise specified a nearest neighbor setting of 10 (default) and P-value ratio cutoff of 0.5 was used. The number of genes used to predict was varied with standard numbers of 50, 40, 30, 20, 10, 5, 2 and 1 genes used. For each number of genes the numbers of correct calls, incorrect calls and non-calls were recorded. Non-calls are cases where no prediction was made because the P-value ratio exceeded the specified P-value ratio cutoff. Calculations were made for overall percent correct calls (number of correct classifications/number or samples), percent correct calls of called samples (number of correct classifications/number of samples with calls) and percent of called samples (samples with calls/number of samples).

For each input list and optimal number of predictive genes (lowest number of genes giving a maximum overall percent of correct calls) additional information was recorded that included the list of specific genes in the optimum predictive set.

Results:

Expression array data were examined for the existence of genes whose expression correlated with histopathology scores. Table 1 in Materials and Methods of Example 1 presents a list of the compounds and dose levels along with the histopathology classification and histopathology severity scores used for this analysis. For each distance measure the probability was adjusted in increments of 0.05 until at least 50 correlating genes were obtained. Lists of correlating genes were obtained using the distance measures described in Materials and Methods. Example sets of correlating genes are provided in Tables 24-25.

The correlating gene lists as well as the entire array gene list were provided as input lists to the GeneSpring Predict Parameter value tool (described in Materials and Methods) that employs a K-means nearest neighbor (knn) predictive model. These lists as well as the entire array gene list were used for each of the five training and test sets defined in Materials and Methods o generate predictions of histopathology classifications of the test sets. Input genes for the Predict Parameter Value feature included all 700 genes in the GenePix file (the Rat CT Array) as well as smaller lists of genes whose expressions correlated with histopathology by the correlation measures described previously. The number of genes used to predict are varied with standard numbers of 50, 40, 30, 20, 10, 5, 2 and 1 genes used. The specified number of predictive genes was varied to obtain an optimum number of predictive genes.

After this was done for 5 training and test sets, all gene lists were then merged to create one aggregate list of predictive genes. Each gene on this aggregate list has predictive value for at least one of the training and test sets because it was observed to contribute to an optimum predictivity for a specific training/test set. The aggregate list was subdivided into smaller lists of genes based on the number of times a gene was predictive for an individual training or test set. For example, if 5 training and test sets were used, genes that were predictive in 5 training and test sets were designated as Combo (combination) 5. Genes that were predictive in only 4 of 5 training and test sets were designated as Combo 4, etc.

A list of predictive genes organized by their occurrence in the separate training and test sets is presented in Table 26.

### EXAMPLE 6

Database:

The database used was as described in Example 1.

Array Data, Normalization and Transformation:

Array data, normalization procedures and transformations used in these analyses are as described in Example 1. Table 36 presents 72 hour gene expression data for the predictive genes. These data can be used with a k-means nearest neighbor prediction model (as available in GeneSpring or other statistical software packages) to make predictions as described in this example.

Class Prediction:

The Predict Parameter Values tool in GeneSpring™ software was used for toxicity class prediction. A description of this tool and the statistical procedures used is provided in Example 1.

Training and Test Data Sets:

The training and test data sets used are those described in the table of Example 5.

Toxicology Classification:

Toxicology classifications used are described in Table 1 of Example 1. In this analysis randomized classifications (same number of “yes” and “no” classifications distributed randomly among the samples) were also used.

Prediction Output and Initial Data Processing:

For each gene list prediction used for evaluation a table of data generated by the Predict Parameter Values tool in GeneSpring™ software was saved which provided for each sample in the test set the actual call (“yes” or “no” for toxicity), the predicted call (“yes”, “no” or no call for toxicity) and the P-value cutoff ratio. This set of data was used to calculate predictive performance measures provided below.

Prediction Measures:

Measures of prediction used for these analyses are generally accepted prediction measures for information about actual and predicted classifications done by a classification system (Venables and Ripley, ibid; Kubat and Matwin, ibid). Results from predictions of a two-class case can be described as a two-class matrix:

Predicted

| Actual | Negative | Positive |
|---|---|---|
| Negative | a | b |
| Positive | c | d |

Standard terms used for prediction are the same as in Example 2. In these analyses cases where no prediction was made because the p-value ratio exceeded the cutoff-value (generally 0.5) the non-call was considered to be incorrect.

Results:

Prediction results for 72 hour expression data using genes identified as predictive are presented in Table 27. These data indicate accuracy in predicting toxicity with 72 hr expression data. Mean accuracy exceeded 0.7 (70% accuracy) for the entire predictive gene list (Combo All) and Combo 4, 3 and 2 sets and 0.55 (55%) for the Combo 1 and 5 gene lists. Mean false positive and false negative values were in the range of 0.2-0.4 for the best predicting gene sets and the geometric mean measures were higher than 0.6 for all gene sets.

Comparison of predictive performance for correct and random classification is given in Table 28.

It is clear from these data that the predictions with accurate classification are much better than predictions with randomized classification. This means that the predictive results are not simply due to chance and large data sets but are due to significant, meaningful predictive association between the gene expression of the predictive genes and the toxicity.

### EXAMPLE 7

Predictive Modeling:

The predictive task with the toxicology gene expression data is a two-class classification problem, where the two classes of possible responses are defined by either hepatocellular necrosis (yes) or absence of hepatocellular necrosis (no). This is an uneven class problem in that the class of yes responses is roughly 20 percent of the data or less in the database tested. A discrimination function can be used to classify a training set. This function can be cross-validated with a testing set, often repeatedly to quantify the mean and variation of the classification error. There are numerous common discrimination functions, and a comparative study of the performance of these functions is useful in determining the best classifier. Additional measures can then. be used to compare the performance of the classifiers. Since the classes are of significantly uneven sizes, use a geometric mean measure (GMM) can be used to compare models, namely, the square root of the product of the true positives and the true negatives.

Common discrimination methods are Fisher's linear discriminant, quadratic discriminant (mahalanobis distance), k-nearest neighbors (knn), logistic discriminant (MacLachlan, 1992), classification trees (or more generally known as recursive partitioning) (Breiman et al., 1984; Clark and Pregibon, 1993; Quinlan and Kaufman, 1988), and neural network classifiers [Ripley, 1996). Most are formula-based such as linear and quadratic discriminant, whereas others are rule-based, such as recursive partitioning, or algorithmically based, such as knn. knn is also database dependent in that a database containing training set is needed to perform nearest neighbor search and classification.

Classifier Models:

A variety of common classification techniques are available. A simple hybrid classifier could be designed and tested, using the knn results, to transform the knn model into a database independent model. This model is termed a centroid model. The centroid model uses the correctly identified test data results from knn and locates a centroid of the subset of k samples that are of the same class for each correctly identified test sample. The centroid is assigned the correct class, and with new test data, a sample is assigned the class of its nearest centroid.

In addition to the knn and centroid models described above, tree, centroid, logistic, and neural network models could also be employed. The neural network is a simple, feed-forward network, allowing skip layers, and with an entropy fitting criterion.

### EXAMPLE 8

Animal Treatment and Tissue Harvest:

Male Sprague-Dawley rats in groups of 3 were treated by intraperitoneal injection with test compounds (thioacetamide, 200 mg/kg and a-naphthylisothiocyanate (ANIT), 100 mg/kg) or only with the vehicle in which the compound was mixed. At specified timepoints (24 h and 72 h) the rats were euthanized and tissues collected. tissues were immediately placed into liquid nitrogen and frozen within 3 minutes of the death of the animal to ensure that mRNA did not degrade. The tissues were sent blinded to be tested. The organs/tissues were then packaged into well-labeled plastic freezer quality bags and stored at −80 degrees until needed for isolation of the mRNA from a portion of the organ/tissue sample.

Gene Expression Measurement:

Isolation of RNA, preparation of cDNA labeled probes and hybridizations procedures were as described in Example 1 Materials and Methods. Probes were hybridized to the rat CT Chip which is the same array as used for the database.

Data Analysis

Array data from the samples was loaded into GeneSpring software using the same procedures as used for the database. No toxicity parameters were entered for these samples. The Predict Parameter Value tool was used to make toxicity predictions using different Combo Gene sets from the 24 hour data and the entire database as the training set. Other values used were 10 nearest neighbors and a p-value ratio cutoff of 0.5.

Results:

Table 29 presents predictions for samples that were external to the database used to derive the predictive genes. The samples were samples from replicate animals treated with thioacetamide or ANIT. One of these compounds (ANIT) is also represented in the database (at a different dose level) and the other compound, thioacetamide, is not in the database. Histopathology conducted on the samples verified that these treatments induced hepatocellular necrosis. Each of the Combo gene sets correctly predicted that these samples had expression patterns indicative of toxicity.

These results demonstrate clearly that the discovered sets of predictive genes in conjunction with the database and K-means nearest neighbor model can accurately predict toxicity from microarray data that is external to the database. Because the database consists mostly of non-toxic samples the prediction of toxicity for these samples is significantly different from what would be expected from chance. It is also noteworthy that five different sets of predictive genes are capable of making accurate predictions.

This result provides a clear example of the predictive utility of this invention.

### EXAMPLE 9

Gene Expression Data:

Gene expression data used for cluster analysis were the 24 hour expression data of the 68 genes of the combined Combo 5, 4, 3 and 2 predictive gene sets. These data are contained in Table 35.

Cluster Analysis:

Cluster analysis tools used in these analyses included K-means and gene tree features of GeneSpring software.

Results:

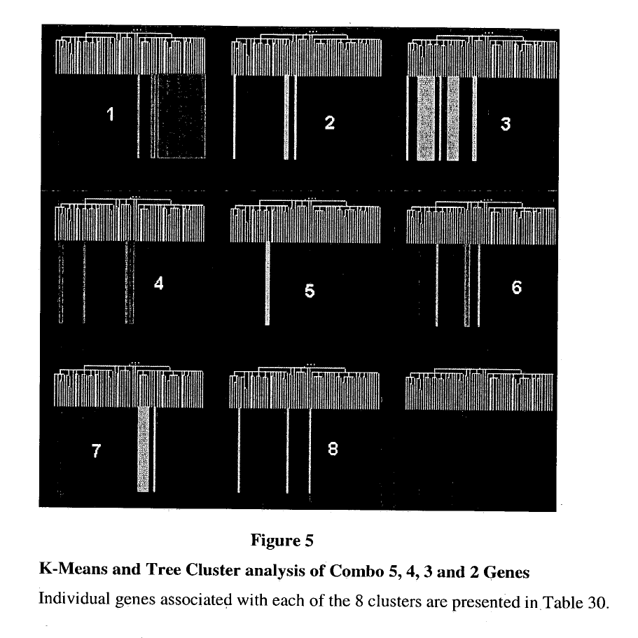

FIG. 5 presents combined results of K-means and gene-tree hierarchical clustering analysis. Combo 5, 4, 3 and 2 (68 genes) were clustered using K-means (number of clusters 8, maximum iteration 100, similarity measure Pearson) and Gene tree (separation ratio 0.5, minimum distance 0.001, similarity measure Pearson). The k-means clusters are colored according to the corresponding set 1 to set 8). The gene on the display from left to right correspond to the gene names top to bottom in the Table 30. These data indicate that the predictive genes can be organized into sets of genes which have similar expression patterns.

It is understood that the examples and embodiments described herein are for illustrative purposes only and that various modifications or changes in light thereof will be suggested to persons skilled in the art and are to be included within the spirit and purview of this application and scope of the appended claims. All publications, patents and patent applications cited herein are hereby incorporated by reference in their entirety for all purposes to the same extent as if each individual publication, patent or patent application were specifically and individually indicated to be so incorporated by reference.

TABLE 1
Compounds, Dose Levels, Liver Pathology
and Abbreviations in the Database

| | Dose | | | Liver* | |
|---|---|---|---|---|---|
| Compound | Level | | Abbreviation | Necrosis | Score** |
| 1-naphthyliso- | 15 | mgkg | ANIT 15 | no | 1 |
| thiocyanate | | | | | |
| 1-naphthyliso- | 60 | mgkg | ANIT 60 | Yes | 2 |
| thiocyanate | | | | | |
| 5-fluorouracil | 13 | mg/kg | 5-FU 13 | no | 1 |
| 5-fluorouracil | 50 | mg/kg | 5-FU 50 | no | 1 |
| acetaminophen | 250 | mg/kg | APAP 250 | no | 1 |
| acetaminophen | 1000 | mg/kg | APAP 1000 | Yes | 2 |
| aflatoxin B1 | 1 | mg/kg | AFLB 1 | Yes | 8 |
| | | | | (no 24 h) | |
| amphotericin B | 5 | mg/kg | AMPB 5 | No | 1 |
| amphotericin B | 20 | mg/kg | AMPB 20 | No | 1 |
| azathioprine | 50 | mg/kg | AZA 50 | No | 1 |
| azathioprine | 200 | mg/kg | AZA 200 | No | 1 |
| benzene | 0.25 | ml/kg | BEN 250 | No | 1 |
| benzene | 1 | ml/kg | BEN 1000 | No | 1 |
| benzo[a]- | 30 | mg/kg | BAP 30 | No | 1 |
| pyrene | | | | | |
| bromobenzene | 0.2 | ml/kg | BRB 200 | Yes | 2 |
| bromobenzene | 0.8 | ml/kg | BRB 800 | Yes | 4 |
| busulfan | 14 | mg/kg | BUS 14 | no | 1 |
| cadmium | 1 | mg/kg | CAD 1 | no | 1 |
| chloride | | | | | |
| cadmium | 2 | mg/kg | CAD 2 | No | 1 |
| chloride | | | | (72 h only) | |
| cadmium | 4 | mg/kg | CAD 4 | Yes | 3 |
| chloride | | | | (6 h only) | |
| carbon | 0.25 | ml/kg | CCL4 250 | Yes | 3 |
| tetrachloride | | | | | |
| carbon | 1 | ml/kg | CCL4 1000 | Yes | 6 |
| tetrachloride | | | | | |
| carmustine | 16 | mg/kg | CAR 16 | no | 1 |
| chloroform | 0.25 | ml/kg | CHCL3 250 | no | 1 |
| chloroform | 0.5 | ml/kg | CHCL3 500 | no | 1 |
| chlorpromazine | 8 | mg/kg | CHLOR 8 | no | 1 |
| chlorpromazine | 30 | mg/kg | CHLOR 30 | no | 1 |
| cisplatin | 2.5 | mg/kg | CIS 2.5 | no | 1 |
| cisplatin | 10 | mg/kg | CIS 10 | no | 1 |
| clofibrate | 75 | mg/kg | CLO 75 | no | 1 |
| clofibrate | 250 | mg/kg | CLO 250 | no | 1 |
| clozapine | 45 | mg/kg | CLOZ 45 | no | 1 |
| clozapine | 180 | mg/kg | CLOZ 180 | no | 1 |
| carboxy | 30 | mg/kg | CMC 30 | no | 1 |
| methyl | | | | | |
| cellulose | | | | | |
| cycloheximide | 0.5 | mg/kg | CHEX 0.5 | no | 1 |
| cycloheximide | 2 | mg/kg | CHEX 2 | no | 1 |
| cyclophos- | 25 | mg/kg | CPHOS 25 | no | 1 |
| phamide | | | | | |
| cyclophos- | 100 | mg/kg | CPHOS 100 | no | 1 |
| phamide | | | | | |
| cyclosporin A | 20 | mg/kg | CYCA 20 | no | 1 |
| cyclosporin A | 80 | mg/kg | CYCA 80 | no | 1 |
| dexamethasone | 8 | mg/kg | DEX 8 | no | 1 |
| dexamethasone | 30 | mg/kg | DEX 30 | no | 1 |
| diflunisal | 25 | mg/kg | DIF 25 | no | 1 |
| diflunisal | 100 | mg/kg | DIF 100 | no | 1 |
| dimethyl- | 20 | mg/kg | DMN 20 | Yes | 9 |
| nitrosamine | | | | | |
| doxorubicin | 12 | mg/kg | DOX 12 | no | 1 |
| erythromycin | 40 | mg/kg | ERY 40 | no | 1 |
| estolate | | | | | |
| erythromycin | 160 | mg/kg | ERY 160 | no | 1 |
| estolate | | | | | |
| estradiol | 0.1 | mg/kg | EST 0.1 | no | 1 |
| estradiol | 0.4 | mg/kg | EST 0.4 | no | 1 |
| ethanol | 2.5 | ml/kg | ETH 2500 | no | 1 |
| gancyclovir | 50 | mg/kg | GAN 50 | no | 1 |
| gancyclovir | 200 | mg/kg | GAN 200 | no | 1 |
| gentamicin | 38 | mg/kg | GEN 38 | no | 1 |
| gentamicin | 150 | mg/kg | GEN 150 | no | 1 |
| hydroxyurea | 250 | mg/kg | HYD 250 | no | 1 |
| hydroxyurea | 1000 | mg/kg | HYD 1000 | no | 1 |
| isoniazid | 50 | mg/kg | ISON 50 | no | 1 |
| isoniazid | 200 | mg/kg | ISON 200 | no | 1 |
| ketoconazole | 20 | mg/kg | KETO 20 | no | 1 |
| ketoconazole | 80 | mg/kg | KETO 80 | no | 1 |
| lipopoly- | 2 | mg/kg | LPS 2 | no | 1 |
| saccharide | | | | | |
| lipopoly- | 8 | mg/kg | LPS 8 | Yes | 6 |
| saccharide | | | | | |
| methotrexate | 1.3 | mg/kg | MET 1.3 | no | 1 |
| methotrexate | 5 | mg/kg | MET 5 | no | 1 |
| naloxone | 45 | ml/kg | NAL 45 | no | 1 |
| naloxone | 180 | mg/kg | NAL 180 | no | 1 |
| phenobarbital | 20 | mg/kg | PBARB 20 | no | 1 |
| phenobarbital | 80 | mg/kg | PBARB 80 | no | 1 |
| phenyl- | 20 | mg/kg | PHEN 20 | no | 1 |
| hydrazine | | | | | |
| phenyl- | 80 | mg/kg | PHEN 80 | no | 1 |
| hydrazine | | | | | |
| polyethylene | 5 | ml/kg | PEG 5000 | no | 1 |
| glycol | | | | | |
| puromycin | 38 | mg/kg | PUR 38 | no | 1 |
| puromycin | 150 | mg/kg | PUR 150 | no | 1 |
| quinidine | 25 | mg/kg | QUIN 25 | no | 1 |
| quinidine | 100 | mg/kg | QUIN 100 | no | 1 |
| streptozotocin | 20 | mg/kg | STRZ 20 | no | 1 |
| streptozotocin | 75 | mg/kg | STRZ 75 | no | 1 |
| tamoxifen | 50 | mg/kg | TAM 50 | no | 1 |
| tamoxifen | 200 | mg/kg | TAM 200 | no | 1 |
| tetracycline | 50 | mg/kg | TET 50 | no | 1 |
| tetracycline | 150 | mg/kg | TET 150 | Yes | 2 |
| theophylline | 25 | mg/kg | THEO 25 | no | 1 |
| theophylline | 100 | mg/kg | THEO 100 | no | 1 |

*Values in parentheses indicate that array data are only available for indicated time points
**Histopathology liver necrosis severity scores. 1 = not remarkable; 2 and higher indicate histopathology of increasing severity

TABLE 2
Distribution of Compounds* in Individual Training and Test
Sets for 24 Hour Liver Data
Training and Test Set 1

| Training Set 1 | Training Set 1 | Test Set 1 | Test Set 1 |
|---|---|---|---|
| Negative | Positive | Negative | Positive |
| 5-FU | ANIT | CAD | CCL4 |
| AMPB | APAP | CAR | LPS |
| AZA | BRB | CHLOR | TET |
| BAP | DMN | CIS | |
| BEN | | CLO | |
| BUS | | DEX | |
| CHCL3 | | EST | |
| CHEX | | GEN | |
| CLOZ | | HYD | |
| CMC | | ISON | |
| CPHOS | | MET | |
| CYCA | | NAL | |
| DIF | | PHEN | |
| DOX | | PUR | |
| ERY | | QUIN | |
| ETH | | STRZ | |
| GAN | | | |
| KETO | | | |
| PBARB | | | |
| PEG | | | |
| TAM | | | |
| THEO | | | |

Training and Test Set 2

| Training Set 2 | Training Set 2 | Test Set 2 | Test Set 2 |
|---|---|---|---|
| Negative | Positive | Negative | Positive |
| 5-FU | BRB | AZA | ANIT |
| AMPB | CCL4 | BAP | APAP |
| BEN | LPS | BUS | DMN |
| CAD | TET | CAR | |
| CHEX | | CHCL3 | |
| CHLOR | | CLO | |
| CIS | | CPHOS | |
| CLOZ | | DIF | |
| CMC | | DOX | |
| CYCA | | ERY | |
| DEX | | GAN | |
| EST | | ISON | |
| ETH | | MET | |
| GEN | | PHEN | |
| HYD | | PUR | |
| KETO | | STRZ | |
| NAL | | | |
| PBARB | | | |
| PEG | | | |
| QUIN | | | |
| TAM | | | |
| THEO | | | |

Training and Test Set 3

| Training Set 3 | TTraining Set 3 | Test Set 3 | Test Set 3 |
|---|---|---|---|
| NNegative | PPositive | NNegative | Positive |
| 5-FU | ANIT | BAP | APAP |
| AMPB | BRB | CAD | CCL4 |
| AZA | DMN | CHEX | TET |
| BEN | LPS | CIS | |
| BUS | | CLO | |
| CAR | | CMC | |
| CHCL3 | | CYCA | |
| CHLOR | | DIF | |
| CLOZ | | ISON | |
| CPHOS | | NAL | |
| DEX | | PEG | |
| DOX | | PUR | |
| ERY | | QUIN | |
| EST | | STRZ | |
| ETH | | TAM | |
| GAN | | THEO | |
| GEN | | | |
| HYD | | | |
| KETO | | | |
| MET | | | |
| PBARB | | | |
| PHEN | | | |

Training and Test Set 4

| Training Set 4 | Training Set 4 | Test Set 4 | Test Set 4 |
|---|---|---|---|
| Negative | Positive | Negative | Positive |
| AZA | APAP | 5-FU | ANIT |
| BAP | CCL4 | AMPB | BRB |
| CAD | DMN | BEN | TET |
| CAR | LPS | BUS | |
| CHEX | | CHCL3 | |
| CHLOR | | CLO | |
| CIS | | CYCA | |
| CLOZ | | DIF | |
| CMC | | DOX | |
| CPHOS | | ERY | |
| ETH | | EST | |
| GAN | | KETO | |
| GEN | | PEG | |
| HYD | | PUR | |
| ISON | | QUIN | |
| MET | | TAM | |
| NAL | | | |
| PBARB | | | |
| PHEN | | | |
| STRZ | | | |
| THEO | | | |
| DEX | | | |

Training and Test Set 5

| Training Set 5 | Training Set 5 | Test Set 5 | Test Set 5 |
|---|---|---|---|
| Negative | Positive | Negative | Positive |
| 5-FU | ANIT | BAP | CCL4 |
| AMPB | APAP | BEN | LPS |
| AZA | BRB | BUS | TET |
| CAD | DMN | CIS | |
| CAR | | DEX | |
| CHCL3 | | DIF | |
| CHEX | | ERY | |
| CHLOR | | EST | |
| CLO | | GEN | |
| CLOZ | | HYD | |
| CMC | | PBARB | |
| CPHOS | | PEG | |
| CYCA | | PUR | |
| DOX | | STRZ | |
| ETH | | TAM | |
| GAN | | THEO | |
| ISON | | | |
| KETO | | | |
| MET | | | |
| NAL | | | |
| PHEN | | | |
| QUIN | | | |

*For abbreviations please see Table 1 (Compound, Dose, Abbreviation, etc.)
**Negative = Compounds that did not elicit histopathology (score = 1)
Positive = Compounds that did elicit histopathology (score of 2 or greater)

TABLE 3
List of Genes, Whose Expression at 24 h Directly Correlates with
Liver Necrosis at 72 h, Ranked by Pearson Correlation Coefficient

| | Correlation |
|---|---|
| Gene | Coefficient |
| Gadd153 | 0.649 |
| Phase-1 RCT-179 | 0.641 |
| Superoxide dismutase Mn | 0.633 |
| Gadd45 | 0.613 |
| Phase-1 RCT-144 | 0.613 |
| Calpactin I heavy chain | 0.611 |
| Phase-1 RCT-207 | 0.603 |
| 14-3-3 zeta | 0.593 |
| Gamma-actin, cytoplasmic | 0.590 |
| Cyclin G | 0.574 |
| Cathepsin L, sequence 2 | 0.572 |
| Macrophage inflammatory protein-2 alpha | 0.566 |
| Phase-1 RCT-68 | 0.560 |
| Zinc finger protein | 0.553 |
| Multidrug resistant protein-2 | 0.546 |
| Phase-1 RCT-225 | 0.545 |
| Melanoma-associated antigen ME491 | 0.544 |
| 60S ribosomal protein L6 | 0.540 |
| Integrin beta1 | 0.539 |
| Organic cation transporter 3 | 0.537 |
| Phase-1 RCT-49 | 0.534 |
| Heme oxygenase | 0.533 |
| Phase-1 RCT-205 | 0.531 |
| Phase-1 RCT-242 | 0.530 |
| Uncoupling protein 2 | 0.528 |
| IgE binding protein | 0.524 |
| Phase-1 RCT-50 | 0.515 |
| Phase-1 RCT-213 | 0.515 |
| Nucleoside diphosphate kinase | 0.512 |
| beta isoform | |
| IkB-a | 0.511 |
| Phase-1 RCT-39 | 0.509 |
| Endogenous retroviral sequence, | 0.508 |
| 5′ and 3′ LTR | |
| Phase-1 RCT-192 | 0.507 |
| Phase-1 RCT-109 | 0.504 |
| Phase-1 RCT-145 | 0.504 |
| Phase-1 RCT-152 | 0.503 |
| Phase-1 RCT-154 | 0.502 |
| Voltage-dependent anion channel (Vdac2) | 0.502 |
| Ubiquitin conjugating enzyme (RAD 6 | 0.499 |
| homologue) | |
| PAR interacting protein | 0.498 |
| Insulin-like growth factor binding | 0.495 |
| protein 1 | |
| Cofilin | 0.493 |
| Ribosomal protein L13A | 0.493 |
| Pyruvate kinase, muscle | 0.493 |
| Beta-actin | 0.492 |
| 60S ribosomal protein L6 (alternate | 0.492 |
| clone 1) | |
| Phase-1 RCT-37 | 0.482 |
| Phase-1 RCT-72 | 0.481 |
| ID-1 | 0.478 |
| Thymosin beta-10 | 0.472 |
| Osteoactivin | 0.470 |
| Multidrug resistant protein-1 | 0.466 |
| Phase-1 RCT-127 | 0.463 |
| p53 | 0.459 |
| Phase-1 RCT-241 | 0.459 |
| Elongation factor-1 alpha | 0.457 |
| Matrix metalloproteinase-1 | 0.457 |
| c-myc | 0.456 |
| Phase-1 RCT-162 | 0.455 |
| Beta-tubulin, class I | 0.454 |
| Interleukin-1 beta | 0.451 |

TABLE 4
List of Genes, Whose Expression at 24 h Inversely
Correlates with Liver Necrosis at 72 h, Ranked
by Spearman Correlation Coefficient

| | Correlation |
|---|---|
| Gene | Coefficient |
| Phase-1 RCT-21 | −0.350 |
| Phase-1 RCT-119 | −0.350 |
| Apolipoprotein C1 | −0.353 |
| Phase-1 RCT-164 | −0.353 |
| {Phase-1 RCT 98} | −0.355 |
| HMG-CoA synthase, mitochondrial | −0.357 |
| {Phase-1 RCT-200} | −0.358 |
| Phase-1 RGT-161 | −0.359 |
| Aldehyde dehydrogenase 2 | −0.359 |
| Phase-1 RCT-117 | −0.359 |
| Phase-1 RCT-270 | −0.359 |
| Octamer binding protein 1 | −0.361 |
| Diazepam binding inhibitor | −0.362 |
| Phase-1 RCT-189 | −0.363 |
| Phase-1 RCT-175 | −0.364 |
| Cytochrome P450 11A1 | −0.365 |
| Phase-1 RCT-123 | −0.365 |
| Phase-1 RCT-239 | −0.365 |
| Phase-1 RCT-64 | −0.367 |
| Phase-1 RCT-8 | −0.371 |
| Phase-1 RCT-131 | −0.374 |
| Preproalbumin, sequence 2 | −0.376 |
| Fatty acid synthase | −0.379 |
| NADP-dependent isocitrate dehydrogenase, | −0.380 |
| cytosolic | |
| Phase-1 RCT-290 | −0.380 |
| Extracellular-signal-regulated kinase 1 | −0.380 |
| ATPase inhibitor (rat mitochondrial IF1 | −0.381 |
| protein) | |
| Phase-1 RCT-40 | −0.381 |
| Stem cell factor | −0.384 |
| Phase-1 RCT-227 | −0.384 |
| Apolipoprotein AII | −0.387 |
| NADH-cytochrome b5 reductase | −0.388 |
| Histidine-rich glycoprotein | −0.390 |
| Phase-1 RCT-280 | −0.390 |
| Methylacyl-CoA racemase alpha | −0.392 |
| Contrapsin-like protease inhibitor | −0.394 |
| (CPi-21) | |
| Phase-1 RCT-209 | −0.394 |
| Glutathione peroxidase | −0.398 |
| Betaine homocysteine methyltransferase | −0.400 |
| (BHMT) | |
| Aquaporin-3 (AQP3) | −0.403 |
| Phase-1 RCT-233 | −0.405 |
| Sterol carrier protein 2 | −0.407 |
| Tryptophan hydroxylase | −0.408 |
| Cytochrome P450 3A1 | −0.409 |
| Phase-1 RCT-83 | −0.411 |
| Senescence marker protein-30 | −0.416 |
| Phase-1 RCT-289 | −0.416 |
| Carbonic anhydrase III, sequence 2 | −0.417 |
| Phase-1 RCT-185 | −0.418 |
| Transthyretin | −0.419 |
| Phase-1 RCT-181 | −0.420 |
| Sodium/bile acid cotransporter | −0.423 |
| Paraoxonase 1 | −0.426 |
| Phase-1 RCT-128 | −0.426 |
| Phase-1 RCT-182 | −0.430 |
| Phase-1 RCT-296 | −0.430 |
| Phase-1 RCT-291 | −0.431 |
| Phase-1 RCT-264 | −0.432 |
| Phase-1 RCT-52 | −0.437 |
| Aldehyde dehydrogenase, microsomal | −0.442 |
| Organic anion transporter 3 | −0.442 |
| Presenilin-1 | −0.447 |
| Phase-1 RCT-102 | −0.449 |
| Phase-1 RCT-89 | −0.449 |
| Phase-1 RCT-218 | −0.450 |
| N-hydroxy-2-acetylaminofluorene | −0.452 |
| sulfotransferase (ST1C1) | |
| Liver fatty acid binding protein | −0.456 |
| Apolipoprotein CIII | −0.456 |
| Phase-1 RCT-88 | −0.457 |
| Phase-1 RCT-168 | −0.457 |
| Alpha 1 - inhibitor III | −0.461 |
| Phase-1 RCT-288 | −0.464 |
| Equilbrative nitrobenzylthioinosine- | −0.465 |
| sensitive nucleoside transporter | |
| Phase-1 RCT-33 | −0.465 |
| Phase-1 RCT-256 | −0.466 |
| Phase-1 RCT-36 | −0.468 |
| Dynamin-1 (D100) | −0.470 |
| L-gulono-gamma-lactone oxidase | −0.472 |
| Phase-1 RCT-38 | −0.477 |
| Phase-1 RCT-214 | −0.478 |
| Carbonic anhydrase III | −0.485 |
| Matrin F/G | −0.489 |
| Phase-1 RCT-92 | −0.492 |
| Hepatic lipase | −0.498 |
| Phase-1 RCT-78 | −0.507 |

TABLE 5
Predictive Genes for 24 Hour Expression Data

| | Combination |
|---|---|
| Gene Name | Category |
| Gamma-actin, cytoplasmic | 5 |
| Matrin F/G | 5 |
| Phase-1 RCT-78 | 5 |
| Cathepsin L, sequence 2 | 4 |
| Gadd45 | 4 |
| Phase-1 RCT-144 | 4 |
| Phase-1 RCT-145 | 4 |
| Phase-1 RCT-50 | 4 |
| Phase-1 RCT-92 | 4 |
| Zinc finger protein | 4 |
| 14-3-3 zeta | 3 |
| Dynamin-1 (D100) | 3 |
| Insulin-like growth factor binding | 3 |
| protein 1 | |
| L-gulono-gamma-lactone oxidase | 3 |
| Ornithine decarboxylase | 3 |
| PAR interacting protein | 3 |
| Phase-1 RCT-128 | 3 |
| Phase-1 RCT-180 | 3 |
| Phase-1 RCT-182 | 3 |
| Phase-1 RCT-207 | 3 |
| Phase-1 RCT-213 | 3 |
| Phase-1 RCT-256 | 3 |
| Phase-1 RCT-258 | 3 |
| Phase-1 RCT-264 | 3 |
| Phase-1 RCT-271 | 3 |
| Phase-1 RCT-288 | 3 |
| Phase-1 RCT-33 | 3 |
| Phase-1 RCT-36 | 3 |
| Phase-1 RCT-38 | 3 |
| Phase-1 RCT-39 | 3 |
| Phase-1 RCT-68 | 3 |
| Phase-1 RCT-89 | 3 |
| Phase-1 RCT-139 | 2 |
| 3-hydroxyisobutyrate dehydrogenase | 2 |
| 60S ribosomal protein L6 | 2 |
| Alpha 1 - inhibitor III | 2 |
| Bax (alpha) | 2 |
| Beta-actin | 2 |
| Carbonic anhydrase III | 2 |
| c-myc | 2 |
| Epidermal growth factor | 2 |
| Equilbrative nitrobenzylthioinosine- | 2 |
| sensitive nucleoside transporter | |
| Heme oxygenase | 2 |
| Hepatic lipase | 2 |
| ID-1 | 2 |
| Insulin-like growth factor binding | 2 |
| protein 3 | |
| Integrin beta1 | 2 |
| N-hydroxy-2-acetylaminofluorene | 2 |
| sulfotransferase (ST1C1) | |
| Organic anion transporter 3 | 2 |
| Paraoxonase 1 | 2 |
| Phase-1 RCT-102 | 2 |
| Phase-1 RCT-117 | 2 |
| Phase-1 RCT-123 | 2 |
| Phase-1 RCT-152 | 2 |
| Phase-1 RCT-179 | 2 |
| Phase-1 RCT-189 | 2 |
| Phase-1 RCT-191 | 2 |
| Phase-1 RCT-241 | 2 |
| Phase-1 RCT-270 | 2 |
| Phase-1 RCT-291 | 2 |
| Voltage-dependent anion channel | 2 |
| (Vdac2) | |
| Phase-1 RCT-296 | 2 |
| Phase-1 RCT-40 | 2 |
| Phase-1 RCT-48 | 2 |
| Phase-1 RCT-49 | 2 |
| Phase-1 RCT-83 | 2 |
| Ribosomal protein S17 | 2 |
| Senescence marker protein-30 | 2 |
| 60S ribosomal protein L6 (alternate | 1 |
| clone 1) | |
| 25-DX | 1 |
| Aflatoxin B1 aldehyde reductase | 1 |
| Aldehyde dehydrogenase, microsomal | 1 |
| Alpha-2-macroglobulin | 1 |
| Apolipoprotein CIII | 1 |
| Argininosuccinate lyase | 1 |
| ATPase inhibitor (rat mitochondrial | 1 |
| IF1 protein) | |
| Beta-tubulin, class I | 1 |
| Calpactin I heavy chain | 1 |
| Carbamyl phosphate synthetase I | 1 |
| Carbonyl reductase | 1 |
| c-H-ras | 1 |
| c-jun | 1 |
| Cofilin | 1 |
| Cyclin G | 1 |
| DNA polymerase beta | 1 |
| Elongation factor-1 alpha | 1 |
| Endogenous retroviral sequence, | 1 |
| 5′ and 3′ LTR | |
| Enolase alpha | 1 |
| Extracellular-signal-regulated | 1 |
| kinase 1 | |
| Fas antigen | 1 |
| Gadd153 | 1 |
| Glucose-regulated protein 78 | 1 |
| IgE binding protein | 1 |
| IkB-a | 1 |
| Insulin-like growth factor I | 1 |
| Liver fatty acid binding protein | 1 |
| Macrophage inflammatory protein-1 | 1 |
| alpha | |
| Macrophage inflammatory protein-2 | 1 |
| alpha | |
| MAP kinase kinase | 1 |
| Matrix metalloproteinase-1 | 1 |
| Melanoma-associated antigen ME491 | 1 |
| Monocyte chemotactic protein receptor | 1 |
| (CCR2) | |
| Multidrug resistant protein-1 | 1 |
| Multidrug resistant protein-2 | 1 |
| NADPH quinone oxidoreductase-1 | 1 |
| (DT-diaphorase) | |
| Nucleoside diphosphate kinase beta | 1 |
| isoform | |
| p53 | 1 |
| Phase-1 RCT 252 | 1 |
| Phase-1 RCT-109 | 1 |
| Phase-1 RCT-12 | 1 |
| Phase-1 RCT-127 | 1 |
| Phase-1 RCT-137 | 1 |
| Phase-1 RCT-15 | 1 |
| Phase-1 RCT-154 | 1 |
| Phase-1 RCT-162 | 1 |
| Phase-1 RCT-168 | 1 |
| Phase-1 RCT-181 | 1 |
| Phase-1 RCT-185 | 1 |
| Phase-1 RCT-192 | 1 |
| Phase-1 RCT-205 | 1 |
| Phase-1 RCT-214 | 1 |
| Phase-1 RCT-225 | 1 |
| Phase-1 RCT-239 | 1 |
| Phase-1 RCT-242 | 1 |
| Phase-1 RCT-37 | 1 |
| Phase-1 RCT-55 | 1 |
| Phase-1 RCT-65 | 1 |
| Phase-1 RCT-72 | 1 |
| Phase-1 RCT-88 | 1 |
| Proliferating cell nuclear antigen gene | 1 |
| Pyruvate kinase, muscle | 1 |
| Ref-1 | 1 |
| Ribosomal protein L13A | 1 |
| Ribosomal protein S8 | 1 |
| Ribosomal protein S9 | 1 |
| Sodium/bile acid cotransporter | 1 |
| Superoxide dismutase Mn | 1 |
| T-cell cyclophilin | 1 |
| Thymosin beta-10 | 1 |
| Transthyretin | 1 |
| Ubiquitin conjugating enzyme (RAD 6 | 1 |
| homologue) | |
| Uncoupling protein 2 | 1 |

* Combination category is the number of training/test set gene list occurrences.

TABLE 6
Randomly Selected Gene Subsets from 24
hour Combo All Gene Set (142 genes)*

Rand 5
Phase-1 RCT-117
Aflatoxin B1 aldehyde reductase
Phase-1 RCT-128
Insulin-like growth factor I
Phase-1 RCT-258
Rand 10
Phase-1 RCT-139
60S ribosomal protein L6
NADPH quinine oxidoreductase-1 (DT-diaphorase)
Liver fatty acid binding protein
MAP kinase kinase
Melanoma-associated antigen ME491
Pyruvate kinase, muscle
Phase-1 RCT-168
Phase-1 RCT-185
T-cell cyclophilin
Rand 15
Phase-1 RCT-192
Phase-1 RCT-191
Phase-1 RCT-15
Phase-1 RCT-189
Multidrug resistant protein-1
Cyclin G
Urinary protein 2 precursor
Phase-1 RCT-271
Phase-1 RCT-185
Phase-1 RCT-139
Phase-1 RCT-109
Phase-1 RCT-55
Phase-1 RCT-258
Phase-1 RCT-33
Argininosuccinate lyase
*Genes were randomly selected from the Combo All list of predictive genes (142 genes) assigning a random number to each gene, sorting by the random number and selecting the appropriate number of sorted genes.

TABLE 7
Randomly Selected Gene Subsets from 24 hour
Combos 5, 4, 3 combined (32 genes)*

Rand 5
Phase-1 RCT-182
Phase-1 RCT-258
Phase-1 RCT-38
Phase-1 RGT-78
Gadd45
Rand 10
Phase-1 RCT-50
Phase-1 RCT-213
Phase-1 RCT-182
Phase-1 RCT-89
Dynamin-1 (D100)
Phase-1 RCT-38
PAR interacting protein
Phase-1 RCT-256
Phase-1 RCT-145
Phase-1 RCT-39
Rand 15
Phase-1 RCT-144
Phase-1 RCT-180
Phase-1 RCT-33
Zinc finger protein
Phase-1 RCT-288
Dynamin-1 (D100)
Phase-1 RCT-39
14-3-3 zeta
Insulin-like growth factor binding protein 1
Phase-1 RCT-78
Ornithine decarboxylase
L-gulono-gamma-lactone oxidase
Cathepsin L, sequence 2
Phase-1 RCT-207
Phase-1 RCT-92
*Genes were randomly selected from the Combo 5, 4, and 3 combined list of predictive genes (32 genes) assigning a random number to each gene, sorting by the random number and selecting the appropriate number of sorted genes.

TABLE 8
Randomly Selected Gene Subsets from 24 hour
All- Predictive (Nonpredictive) Genes

All-Pred 10 genes
Phenylalanine hydroxylase
Colony-stimulating factor-1
Ciliary neurotrophic factor
Ribosomal protein L13
S-adenosylmethionine decarboxylase
Notch 1
Phase-1 RCT-91
CTP: phosphocholine cytidylyltransferase
Cytochrome P450 1A1
Phase-1 RCT-60
All-Pred 5 genes
Cellular nucleic acid binding protein (CNBP)
VL30 element
Hemoglobin alpha 1 chain (alternate clone)
Complement component C3
Thrombomodulin
All-Pred 15 genes
Nucleosome assembly protein
Neutral endopeptidase 24.11 (enkephalinase)
Cyclin D1
Lactate dehydrogenase-B
Selenoprotein P
Clusterin
Biliverdin reductase
Phase-1 RCT-79
Caspase 3
Adrenomedullin
Ribosomal protein L13
Cytochrome P450 2C39 (alternate clone 2)
Phase-1 RCT-277
Carnitine palmitoyl-CoA transferase
Cytochrome P450 2D18

TABLE 9
Liver Toxicity Individual Sample Prediction Values for 24 Hour Data
Predictive Genes (Combined List and Subsets)

Prediction Measure*

| | | | | | |
|---|---|---|---|---|---|
| | Number | | False | False | Geometric |
| Gene Set | of Genes | Accuracy** | Positive** | Negative** | Mean** |

| | | | | | |
|---|---|---|---|---|---|
| Combo All | 142 | 0.924 (0.872-0.960) | 0.074 (0.034-0.106) | 0.100 (0.000-0.333) | 0.917 (0.772-0.956) |
| Combo 5 | 3 | 0.896 (0.837-0.961) | 0.105 (0.033-0.185) | 0.089 (0.000-0.167) | 0.901 (0.868-0.941) |
| Combo 4 | 7 | 0.857 (0.796-0.913) | 0.146 (0.079-0.220) | 0.128 (0.083-0.222) | 0.862 (0.800-0.915) |
| Combo 3 | 22 | 0.865 (0.755-0.923) | 0.145 (0.076-0.271) | 0.050 (0.000-0.083) | 0.900 (0.854-0.954) |
| Combo 2 | 36 | 0.912 (0.851-0.950) | 0.088 (0.045-0.129) | 0.100 (0.000-0.333) | 0.904 (0.762-0.955) |
| Combo 1 | 74 | 0.894 (0.853-0.941) | 0.094 (0.056-0.122) | 0.206 (0.000-0.444) | 0.844 (0.705-0.949) |

*Prediction measures are given as means and range of values (in parentheses) for five training/test sets
using 24 hour array data and gene lists as presented in Table . Unit of prediction
was the animal and the predictive classification was for liver necrosis observed at 72 hours after
treatment.
**Standard prediction measures were used as defined in Materials and Methods. These include:

| | |
|---|---|
| Accuracy | proportion of total number of predictions that are correct |
| False positive rate | proportion of negative cases that are incorrectly classified as positive |
| False negative rate | proportion of positive cases that are incorrectly classified as negative |
| Geometric mean | performance measure that takes into account proportion of positive and |
| | negative cases |

TABLE 10
Liver Toxicity Compound-Dose Prediction Values for 24
Hour Data Predictive Genes (Combined List and Subsets)

| | No. | Prediction Measure* | | | |
|---|---|---|---|---|---|
| Gene Set | Genes | Accuracy** | False Positive** | False Negative** | Geometric Mean** |
| Combo All | 142 | 0.935 (0.879-0.971) | 0.066 (0.032-0.100) | 0.067 (0.000-0.333) | 0.932 (0.775-0.984) |
| Combo 5 | 3 | 0.941 (0.879-1.000) | 0.065 (0.000-0.133) | 0.000 (0.000-0.000) | 0.966 (0.931-1.000) |
| Combo 4 | 7 | 0.912 (0.879-0.943) | 0.085 (0.032-0.133) | 0.117 (0.000-0.333) | 0.895 (0.775-0.967) |
| Combo 3 | 22 | 0.905 (0.758-0.971) | 0.105 (0.032-0.267) | 0.000 (0.000-0.000) | 0.945 (0.856-0.984) |
| Combo 2 | 36 | 0.947 (0.909-0.971) | 0.059 (0.032-0.100) | 0.000 (0.000-0.000) | 0.970 (0.949-0.984) |
| Combo 1 | 44 | 0.936 (0.879-0.971) | 0.065 (0.032-0.100) | 0.067 (0.000-0.333) | 0.932 (0.775-0.984) |
| *Prediction measures are given as means and range of values (in parentheses) for five training/test sets using 24 hour | | | | | |
| # array data and gene lists as presented in Table 35 and Table 5. Unit of prediction was compound-dose level and the predictive | | | | | |
| # classification was for liver necrosis observed at 72 hours after treatment. Prediction for compound-dose was based on a | | | | | |
| # majority of individual animal calls. In cases where there were an equal number of opposing calls or no calls a no-call | | | | | |
| # was assigned to the compound-dose level. | | | | | |
| **Standard prediction measures were used as defined in Materials and Methods. As described in Materials and Methods in | | | | | |
| # cases where no prediction was made because the p-value ratio exceeded the cutoff-value (generally 0.5) the non-call was | | | | | |
| # considered to be incorrect. | | | | | |

TABLE 11
Liver Toxicity Compound Prediction Values for 24 Hour
Data Predictive Genes (Combined List and Subsets)

| | Number | Prediction Measure* | | | |
|---|---|---|---|---|---|
| Gene Set | of Genes | Accuracy** | False Positive** | False Negative** | Geometric Mean** |
| Combo All | 142 | 0.937 (0.842-1.000) | 0.063 (0.000-0.125) | 0.067 (0.000-0.333) | 0.934 (0.764-1.000) |
| Combo 5 | 3 | 0.947 (0.895-1.000) | 0.063 (0.000-0.125) | 0.000 (0.000-0.000) | 0.968 (0.935-1.000) |
| Combo 4 | 7 | 0.926 (0.842-1.000) | 0.063 (0.000-0.125) | 0.133 (0.000-0.333) | 0.898 (0.764-1.000) |
| Combo 3 | 22 | 0.937 (0.842-1.000) | 0.075 (0.000-0.188) | 0.000 (0.000-0.000) | 0.961 (0.901-1.000) |
| Combo 2 | 36 | 0.958 (0.895-1.000) | 0.050 (0.000-0.125) | 0.000 (0.000-0.000) | 0.974 (0.935-1.000) |
| Combo 1 | 44 | 0.947 (0.842-1.000) | 0.050 (0.000-0.125) | 0.067 (0.000-0.333) | 0.940 (0.764-1.000) |
| *Prediction measures are given as means and range of values (in parentheses) for five training/test sets using 24 hour | | | | | |
| # array data and gene lists as presented in Table 35 and Table 5. Unit of prediction was the compound and the predictive | | | | | |
| # classification was for liver necrosis observed at 72 hours after treatment. Compounds were considered toxic if any | | | | | |
| # compound-dose level for that compound was predicted as toxic. | | | | | |
| **Standard prediction measures were used as defined in Materials and Methods. As described in Materials and Methods in | | | | | |
| # cases where no prediction was made because the p-value ratio exceeded the cutoff-value (generally 0.5) the non-call was | | | | | |
| # considered to be incorrect. | | | | | |

TABLE 12
Individual Gene Predictions: Combo 5

| | Overall Correct Calls (%) | | | |
|---|---|---|---|---|
| Gene Name | Mean | s.d. | min | max |
| Gamma-actin, cytoplasmic | 89.2 | 4.5 | 84.0 | 95.1 |
| Matrin F/G | 82.0 | 8.1 | 74.8 | 91.1 |
| RCT-078 | 76.5 | 18.8 | 50.5 | 91.1 |
| Average Individual Combo 5 | 82.6 | 10.5 | 69.8 | 92.4 |
| Minimum Individual Combo 5 | 76.5 | 4.5 | 50.5 | 91.1 |
| Maximum Individual Combo 5 | 89.2 | 18.8 | 84.0 | 95.1 |

TABLE 13
Individual Gene Predictions: Combo 4

| | Overall Correct Calls (%) | | | |
|---|---|---|---|---|
| Gene Name | Mean | s.d. | min | max |
| Gadd 45 | 80.9 | 8.7 | 70.2 | 92.1 |
| Cathepsin L | 77.3 | 9.1 | 67.0 | 90.0 |
| Zinc Finger Protein | 85.1 | 9.6 | 70.2 | 93.1 |
| RCT-144 | 90.6 | 2.8 | 86.5 | 94.1 |
| RCT-145 | 77.6 | 5.3 | 69.1 | 82.3 |
| RCT-50 | 69.0 | 8.3 | 58.7 | 80.6 |
| RCT-92 | 85.0 | 4.3 | 80.4 | 89.4 |
| Average Combo 4 | 80.8 | 6.9 | 71.7 | 88.8 |
| Minimum Individual Combo 4 | 69.0 | 2.8 | 58.7 | 80.6 |
| Maximum Individual Combo 4 | 90.6 | 9.6 | 86.5 | 94.1 |

TABLE 14
Individual Gene Predictions: Combo 3

| | Overall Correct Calls (%) | | | |
|---|---|---|---|---|
| Gene Name | Mean | s.d. | min | max |
| 14-3-3 zeta | 77.8 | 8.5 | 66.0 | 84.2 |
| Dynamin-1 (D100) | 51.0 | 19.0 | 30.1 | 81.7 |
| Insulin-like growth factor | 73.6 | 4.8 | 69.2 | 81.4 |
| binding protein 1 | | | | |
| L-gulono-gamma-lactone oxidase | 82.1 | 16.7 | 52.4 | 91.1 |
| Ornithine decarboxylase | 75.3 | 11.6 | 55.3 | 84.5 |
| PAR interacting protein | 81.8 | 3.9 | 77.9 | 88.2 |
| Phase-1 RCT-128 | 58.2 | 24.9 | 27.7 | 85.6 |
| Phase-1 RCT-180 | 62.3 | 7.2 | 52.1 | 71.6 |
| Phase-1 RCT-182 | 56.3 | 30.7 | 28.4 | 90.4 |
| Phase-1 RCT-207 | 77.0 | 7.7 | 69.6 | 89.4 |
| Phase-1 RCT-213 | 74.3 | 4.8 | 68.1 | 80.8 |
| Phase-1 RCT-256 | 67.3 | 20.2 | 41.5 | 86.1 |
| Phase-1 RCT-258 | 81.5 | 7.1 | 71.3 | 88.3 |
| Phase-1 RCT-264 | 65.8 | 26.7 | 28.7 | 88.4 |
| Phase-1 RCT-271 | 65.2 | 28.5 | 34.0 | 91.1 |
| Phase-1 RGT-288 | 65.3 | 30.4 | 26.7 | 88.5 |
| Phase-1 RCT-33 | 69.4 | 27.0 | 38.6 | 90.4 |
| Phase-1 RCT-36 | 77.2 | 27.6 | 27.9 | 91.1 |
| Phase-1 RCT-38 | 57.9 | 21.5 | 37.2 | 83.2 |
| Phase-1 RCT-39 | 78.4 | 8.5 | 71.6 | 93.1 |
| Phase-1 RCT-68 | 67.5 | 13.2 | 52.5 | 88.5 |
| Phase-1 RCT-89 | 69.5 | 16.1 | 45.2 | 86.2 |
| Average Individual Combo 3 | 69.8 | 16.7 | 48.7 | 86.5 |
| Minimum Individual Combo 3 | 51.0 | 3.9 | 26.7 | 71.6 |
| Maximum Individual Combo 3 | 82.1 | 30.7 | 77.9 | 93.1 |

TABLE 15
Liver Toxicity Compound-Dose Prediction Values for 24 Hour Data with Random Gene Subsets

| | Random | Prediction Measure* | | | |
|---|---|---|---|---|---|
| Gene Set | Subset | Accuracy** | False Positive** | False Negative** | Geometric Mean** |
| Combo All | 15 | 0.888 (0.812-0.943) | 0.123 (0.065-0.200) | 0 | 0.936 (0.894-0.967) |
| Combo All | 10 | 0.815 (0.750-0.886) | 0.174 (0.129-0.258) | 0.3 (0-1.0) | 0.670 (0-0.933) |
| Combo All | 5 | 0.889 (0.857-0.914) | 0.123 (0.097-0.161) | 0 | 0.936 (0.916-0.950) |
| Combo 5 4 3 | 15 | 0.886 (0.719-0.972) | 0.124 (0.031-0.300) | 0 | 0.934 (0.837-0.984) |
| Combo 5 4 3 | 10 | 0.884 (0.829-0.941) | 0.129 (0.065-0.194) | 0 | 0.933 (0.898-0.967) |
| Combo 5 4 3 | 5 | 0.824 (0.8-0.844) | 0.181 (0.161-0.194) | 0.117 (0-0.333) | 0.847 (0.748-0.913) |
| *Randomly selected sets of genes derived from the Combo sets are described in Tables 1-2. | | | | | |
| *Prediction measures are given as means and range of values (in parentheses) for five training/test sets using 24 hour | | | | | |
| # array data and random subsets of genes as presented in Table 35, Table 6, and Table 7. Unit of prediction was compound-dose | | | | | |
| # and the predictive classification was for liver necrosis observed at 72 hours | | | | | |
| after treatment. | | | | | |
| **Standard prediction measures were used as defined in Materials and Methods. As described in Materials and Methods in | | | | | |
| # cases where no prediction was made because the p-value ratio exceeded the cutoff-value (generally 0.5) the non-call was | | | | | |
| # considered to be incorrect. | | | | | |

TABLE 16
Comparison of Predictivity for Correct Liver Toxicity
Classification and Random Classification Using Combo
Gene Sets and Random Subsets and 24 h data

| | | | | | |
|---|---|---|---|---|---|
| Combo All | All Genes | 92.4 | (87.2-96.0) | 26.8 | (20.4-35.6) |
| | 5 genes | 83.9 | (80.1-86.1) | 29.9 | (15.5-49.0) |
| | 10 genes | 78.0 | (74.5-81.6) | 28.2 | (18.1-33.0) |

| | | Accuracy | Accuracy |
|---|---|---|---|
| | | Correct | Random |
| Gene | Gene | Classification** | Classification** |

| List* | Subset* | Mean | Min-Max | Mean | Min.-Max. |
|---|---|---|---|---|---|
| | 15 genes | 86.2 | (78.7-90.3) | 25.0 | (17.0-36.5) |
| Combo 5 4 3 | All Genes | 91.0 | (86.2-95) | 27.4 | (18.1-36.6) |
| | 5 genes | 79.9 | (72.8-91.3) | 29.4 | (26.5-34.0) |
| | 10 genes | 84.7 | (81.6-90.1) | 28.2 | (24.0-32.7) |
| | 15 genes | 82.5 | (72.3-91.3) | 25.0 | (13.8-30.8) |
| Combo 5 | All Genes | 89.6 | (83.7-96.1) | 25.2 | (22.8-28.2) |
| Combo 4 | All Genes | 85.7 | (79.6-91.3) | 26.6 | (17.0-41.3) |
| Combo 3 | All Genes | 86.5 | (75.5-92.3) | 27.5 | (21.3-34.7) |
| Combo 2 | All Genes | 91.2 | (85.1-95.0) | 23.9 | (18.1-29.8) |
| Combo 1 | All Genes | 89.4 | (85.3-94.1) | 24.2 | (18.8-34.0) |
| All - Predict | 5 genes | 47.4 | (34.3-63.5) | 25.3 | (19.8-30.8) |
| | 10 genes | 67.4 | (59.2-80.1) | 24.7 | (14.6-30.9) |
| | 15 genes | 45.9 | (32.7-63.5) | 26.5 | (15.8-33.3) |
| * Combo Gene Lists as in Example 1, Table 1. For Combo lists all genes were used or random subsets as in Tables 1-3. | | | | | |
| # All-Pred used genes randomly elected from genes that were present on the array but not in the preditive list. | | | | | |
| **Accuracy = proportion of the total number of predictions that are correct. Non-calls are counted as incorrect | | | | | |
| # predictions. Accuracy was calculated for correct classifications of liver toxicity assigned to the samples and for | | | | | |
| # randomized classifications in the same proportions as the correct classifications. Values presented are the mean accuracy | | | | | |
| # values for 5 training/test sets with minimum and maximum accuracy values. | | | | | |

TABLE 17
Distribution of Compounds* in Individual Training and Test
Sets for 6 Hour Liver Data
Training and Test Set 1

| Training Set 1 | Training Set 1 | Test Set 1 | Test Set 1 |
|---|---|---|---|
| Negative | Positive | Negative | Positive |
| 5-FU | AFLB | BUS | APAP |
| AMPB | ANIT | CHCL3 | CCL4 |
| AZA | BRB | CHEX | TET |
| BAP | CAD | CMC | |
| BEN | DMN | CPHOS | |
| CAR | LPS | GAN | |
| CHLOR | | HYD | |
| CIS | | ISON | |
| CLO | | MET | |
| CLOZ | | PEG | |
| CYCA | | PUR | |
| DEX | | TAM | |
| DIF | | | |
| DOX | | | |
| ERY | | | |
| EST | | | |
| ETH | | | |
| GEN | | | |
| KETO | | | |
| NAL | | | |
| PBARB | | | |
| PHEN | | | |
| QUIN | | | |
| STRZ | | | |
| THEO | | | |

Training and Test Set 2

| Training Set 2 | Training Set 2 | Test Set 2 | Test Set 2 |
|---|---|---|---|
| Negative | Positive | Negative | Positive |
| AMPB | APAP | 5-FU | AFLB |
| AZA | BRB | BEN | ANIT |
| BAP | CCL4 | CHCL3 | CAD |
| BUS | DMN | CHLOR | |
| CAR | LPS | CIS | |
| CHEX | TET | CLOZ | |
| CLO | | CMC | |
| DEX | | CPHOS | |
| DIF | | CYCA | |
| DOX | | KETO | |
| ERY | | NAL | |
| EST | | QUIN | |
| ETH | | | |
| GAN | | | |
| GEN | | | |
| HYD | | | |
| ISON | | | |
| MET | | | |
| PBARB | | | |
| PEG | | | |
| PHEN | | | |
| PUR | | | |
| STRZ | | | |
| TAM | | | |
| THEO | | | |

Training and Test Set 3

| Training Set 3 | Training Set 3 | Test Set 3 | Test Set 3 |
|---|---|---|---|
| Negative | Positive | Negative | Positive |
| AMPB | AFLB | 5-FU | CCL4 |
| AZA | ANIT | CHEX | DMN |
| BAP | APAP | CMC | LPS |
| BEN | BRB | CPHOS | |
| BUS | CAD | CYCA | |
| CHCL3 | TET | DOX | |
| CHLOR | | MET | |
| CIS | | NAL | |
| CLO | | PHEN | |
| CLOZ | | PUR | |
| DEX | | QUIN | |
| DIF | | THEO | |
| ERY | | | |
| EST | | | |
| ETH | | | |
| GAN | | | |
| GEN | | | |
| HYD | | | |
| ISON | | | |
| KETO | | | |
| PBARB | | | |
| PEG | | | |
| STRZ | | | |
| TAM | | | |
| CAR | | | |

Training and Test Set 4

| Training Set 4 | Training Set 4 | Test Set 4 | Test Set 4 |
|---|---|---|---|
| Negative | Positive | Negative | Positive |
| 5-FU | ANIT | AZA | AFLB |
| AMPB | APAP | BAP | CCL4 |
| BEN | BRB | CHEX | TET |
| BUS | CAD | CLOZ | |
| CAR | DMN | DIF | |
| CHCL3 | LPS | DOX | |
| CHLOR | | ETH | |
| CIS | | HYD | |
| CLO | | PEG | |
| CMC | | PHEN | |
| CPHOS | | PUR | |
| CYCA | | QUIN | |
| DEX | | | |
| ERY | | | |
| EST | | | |
| GAN | | | |
| GEN | | | |
| ISON | | | |
| KETO | | | |
| MET | | | |
| NAL | | | |
| PBARB | | | |
| STRZ | | | |
| TAM | | | |
| THEO | | | |

Training and Test Set 5

| Training Set 5 | Training Set 5 | Test Set 5 | Test Set 5 |
|---|---|---|---|
| Negative | Positive | Negative | Positive |
| 5-FU | AFLB | AZA | BRB |
| AMPB | ANIT | BAP | DMN |
| BEN | APAP | BUS | LPS |
| CAR | CAD | CLO | |
| CHCL3 | CCL4 | CLOZ | |
| CHEX | TET | DOX | |
| CHLOR | | GEN | |
| CIS | | MET | |
| CMC | | NAL | |
| CPHOS | | PEG | |
| CYCA | | PHEN | |
| DIF | | QUIN | |
| ERY | | | |
| EST | | | |
| ETH | | | |
| GAN | | | |
| HYD | | | |
| ISON | | | |
| KETO | | | |
| PBARB | | | |
| PUR | | | |
| STRZ | | | |
| TAM | | | |
| THEO | | | |
| DEX | | | |

*For abbreviations please see Table 1 (Compound, Dose, Abbreviation, etc.)
**Negative = Compounds that did not elicit histopathology (score = 1)
Positive = Compounds that did elicit histopathology (score of 2 or greater)

TABLE 18
List of Genes, Whose Expression at 6 h Directly
Correlates with Liver Hepatocellular Necrosis at
72 h, Ranked by Pearson Correlation Coefficient

| | Correlation |
|---|---|
| Gene | Coefficient |
| Alpha-tubulin | 0.6309915 |
| Superoxide dismutase Mn | 0.6104141 |
| Cathepsin L | 0.6078458 |
| Gadd45 | 0.5948032 |
| ID-1 | 0.5895025 |
| Argininosuccinate lyase | 0.5767352 |
| c-fos | 0.5752904 |
| Beta-actin, sequence 2 | 0.5710737 |
| c-H-ras | 0.5661596 |
| Phase-1 RCT-211 | 0.5653724 |
| Thymosin beta-10 | 0.5610229 |
| Gadd153 | 0.5517636 |
| Uncoupling protein 2 | 0.5497988 |
| Heme binding protein 23 | 0.5460188 |
| Ribosomal protein L13A | 0.5443944 |
| alpha-1,2-fucosyltransferase | 0.543632 |
| Aldehyde dehydrogenase 2 | 0.5385723 |
| Phase-1 RCT-50 | 0.5211563 |
| Phase-1 RCT-109 | 0.5203465 |
| Ecto-ATPase | 0.5152093 |
| Phase-1 RCT-24 | 0.5125332 |
| Bax (alpha) | 0.5095243 |
| Phase-1 RCT-12 | 0.5075572 |
| Bcl-2 | 0.5068672 |
| Phase-1 RCT-49 | 0.5036029 |
| Beta-tubulin, class I | 0.4991521 |
| Calreticulin | 0.4985017 |
| Multidrug resistant protein-3 | 0.4938303 |
| ADP-ribosylation factor-like protein ARL184 | 0.490394 |
| Transferrin | 0.4883213 |
| Cathepsin L, sequence 2 | 0.4877807 |
| Diacylglycerol kinase zeta | 0.4854465 |
| Gamma-glutamyl transpeptidase | 0.4848459 |
| Phase-1 RCT-111 | 0.4843905 |
| 14-3-3 zeta | 0.4822279 |
| Dynein light chain 1 | 0.4804166 |
| Insulin-like growth factor binding protein 1 | 0.479885 |
| Phase-1 RCT-281 | 0.475073 |
| Thiol-specific antioxidant (natural killer cell-enhancing | 0.4740617 |
| factor B) | |
| Cyclin dependent kinase 4 | 0.46983 |
| Phase-1 RCT-68 | 0.4668504 |
| Phase-1 RCT-144 | 0.4655095 |
| MHC class I antigen RT1.A1(f) alpha-chain | 0.4641322 |
| c-jun | 0.4638237 |
| Macrophage inflammatory protein-2 alpha | 0.4544662 |
| Superoxide dismutase Cu/Zn | 0.448226 |
| Stathmin | 0.4478989 |
| Phase-1 RCT-179 | 0.447854 |
| Phase-1 RCT-103 | 0.4475651 |
| Insulin-like growth factor binding protein 5 | 0.4431518 |
| Matrix metalloproteinase-1 | 0.4405304 |
| Pyruvate kinase, muscle | 0.4402392 |
| Glyceraldehyde 3-phosphate dehydrogenase | 0.4401766 |
| Hypoxanthine-guanine phosphoribosyltransferase | 0.4357165 |
| Phase-1 RCT-221 | 0.4341553 |
| Cyclin E | 0.4337104 |
| Peroxisomal 3-ketoacyl-CoA thiolase 2 | 0.4302424 |
| Phase-1 RCT-27 | 0.4273592 |
| Sorbitol dehydrogenase | 0.4245579 |
| Phase-1 RCT-198 | 0.4232769 |
| Phase-1 RCT-43 | 0.4216533 |
| Ornithine decarboxylase | 0.4216079 |
| Alpha-fibrinogen | 0.4215826 |
| Phase-1 RCT-53 | 0.4214711 |
| Phase-1 RCT-147 | 0.4214167 |
| Peroxisomal 3-ketoacyl-CoA thiolase 1 | 0.4176134 |
| Voltage-dependent anion channel 2 (Vdac2) | 0.4174675 |
| Glutathione reductase | 0.4137496 |
| Tryptophan hydroxylase | 0.4123288 |
| Phase-1 RCT-240 | 0.4111351 |
| Zinc finger protein | 0.4091316 |
| Phase-1 RCT-228 | 0.4057419 |
| Phase-1 RCT-14 | 0.4046313 |

TABLE 19
List of Genes, Whose Expression at 6 h Inversely
Correlates with Liver Necrosis at 72 h, Ranked
by Spearman Correlation Coefficient

| | Correlation |
|---|---|
| Gene | Coefficient |
| Phase-1 RCT-36 | −0.1515 |
| Phase-1 RCT-92 | −0.15161 |
| Phase-1 RCT-143 | −0.15557 |
| Sarcoplasmic reticulum calcium ATPase | −0.15628 |
| Cyclin dependent kinase 2 | −0.15633 |
| Gamma-glutamyl transpeptidase | −0.15636 |
| Phase-1 RCT-285 | −0.1569 |
| Phase-1 RCT-292 | −0.15855 |
| Carbamyl phosphate synthetase I | −0.15946 |
| Monoamine oxidase B | −0.16151 |
| Phase-1 RCT-61 | −0.16227 |
| 3-hydroxyisobutyrate dehydrogenase | −0.1642 |
| Cytochrome P450 2C11 | −0.16488 |
| Phase-1 RCT-164 | −0.16502 |
| Vesicular monoamine transporter (VMAT) | −0.1656 |
| Aspartate aminotransferase, mitochondrial | −0.16581 |
| Axin | −0.16584 |
| Phase-1 RCT-13 | −0.16715 |
| N-hydroxy-2-acetylaminofluorene sulfotransferase (ST1C1) | −0.16724 |
| Oxygen regulated protein 150 | −0.16861 |
| Phase-1 RCT-177 | −0.17261 |
| Diacylglycerol kinase zeta | −0.17336 |
| Very long-chain acyl-CoA synthetase | −0.17338 |
| Phase-1 RCT-277 | −0.17348 |
| Phase-1 RCT-256 | −0.17456 |
| Equilbrative nitrobenzylthioinosine-sensitive | −0.17592 |
| nucleoside transporter | |
| H-rev107 | −0.17721 |
| PTEN/MMAC1 | −0.17816 |
| Phase-1 RCT-289 | −0.17819 |
| Phase-1 RCT-271 | −0.17868 |
| Cyclin D3 | −0.17914 |
| Phase-1 RCT-280 | −0.17953 |
| Phase-1 RCT-209 | −0.18117 |
| Malate dehydrogenase, cytosolic | −0.18371 |
| Extracellular-signal-regulated kinase 1 | −0.1844 |
| NADH-cytochrome b5 reductase | −0.18481 |
| Phase-1 RCT-288 | −0.18493 |
| Phase-1 RCT-82 | −0.18497 |
| Phase-1 RCT-10 | −0.18613 |
| Organic anion transporter 3 | −0.18615 |
| Phase-1 RCT-52 | −0.18746 |
| Phase-1 RCT-287 | −0.19026 |
| Carbonic anhydrase II | −0.19132 |
| Complement component C3 | −0.1918 |
| Protein tyrosine phosphatase alpha | −0.1925 |
| Aldehyde dehydrogenase, microsomal | −0.19284 |
| D-dopachrome tautomerase | −0.19309 |
| Phase-1 RCT-218 | −0.19413 |
| Phase-1 RCT-89 | −0.19423 |
| Cytochrome P450 1A2 | −0.19844 |
| Phase-1 RCT-173 | −0.20095 |
| Phase-1 RCT-119 | −0.20097 |
| Matrin F/G | −0.20244 |
| Phase-1 RCT-102 | −0.20574 |
| Cyclin dependent kinase 4 | −0.20718 |
| Hydroxysteroid sulfotransferase a | −0.20766 |
| Lysyl hydroxylase | −0.20785 |
| Phase-1 RCT-184 | −0.2098 |
| 8-oxoguanine DNA glycosylase | −0.2129 |
| JNK1 stress activated protein kinase | −0.21334 |
| Glutamine synthetase | −0.2145 |
| Phase-1 RCT-291 | −0.2172 |
| S-adenosylmethionine decarboxylase | −0.22017 |
| NADP-dependent isocitrate dehydrogenase, | −0.22819 |
| cytosolic | |
| Phase-1 RCT-182 | −0.2292 |
| DNA topoisomerase I | −0.23083 |
| Selenoprotein P | −0.23114 |
| C4b-binding protein | −0.23274 |
| Alcohol dehydrogenase 1 | −0.23292 |
| Phase-1 RCT-83 | −0.23342 |
| Phase-1 RCT-78 | −0.23557 |
| 17-beta hydroxysteroid dehydrogenase, type 2 | −0.23694 |
| Sterol carrier protein 2 | −0.23977 |
| Iron-responsive element-binding protein | −0.24103 |
| Peroxisomal multifunctional enzyme type II | −0.24167 |
| Phase-1 RCT-168 | −0.24388 |
| Phase-1 RCT-270 | −0.24473 |
| 3-beta-hydroxysteroid dehydrogenase (HSD3B1) | −0.25101 |
| Acetyl-CoA carboxylase | −0.2543 |
| Emerin | −0.25719 |
| Phase-1 RCT-73 | −0.26044 |
| Nucleosome assembly protein | −0.26213 |
| Cytochrome P450 2E1 | −0.26809 |
| Thymidylate synthase | −0.27492 |
| Phase-1 RCT-161 | −0.28042 |
| Cholesterol 7-alpha-hydroxylase (P450 VII) | −0.28206 |
| Phase-1 RCT-40 | −0.28754 |
| Stem cell factor | −0.28765 |
| Glucokinase | −0.30523 |
| Tryptophan hydroxylase | −0.30775 |
| Phase-1 RCT-214 | −0.31173 |
| Carbonic anhydrase III | −0.31836 |
| Senescence marker protein-30 | −0.37821 |

TABLE 20
List of genes whose expression at 6 hours is
predictive of liver toxicity at 72 hours

| | Combination |
|---|---|
| Gene Name | Category* |
| Argininosuccinate lyase | 5 |
| Cathepsin L, sequence 2 | 5 |
| c-myc | 5 |
| Gadd153 | 5 |
| Gadd45 | 5 |
| Heme oxygenase | 5 |
| Insulin-like growth factor binding protein 1 | 5 |
| NIPK | 5 |
| Phase-1 RCT-207 | 5 |
| Phase-1 RCT-50 | 5 |
| Alpha-2-macroglobulin, sequence 2 | 4 |
| c-jun | 4 |
| Phase-1 RCT-127 | 4 |
| Phase-1 RCT-242 | 4 |
| Phase-1 RCT-82 | 4 |
| Pyruvate kinase, muscle | 4 |
| Zinc finger protein | 4 |
| Cyclin dependent kinase 4 | 3 |
| Focal adhesion kinase (pp125FAK) | 3 |
| Glucokinase | 3 |
| Integrin beta1 | 3 |
| Interferon related developmental regulator IFRD1 | 3 |
| (PC4) | |
| NGF-inducible anti-proliferative putative secreted | 3 |
| protein (PC3) | |
| Peroxisomal multifunctional enzyme type II | 3 |
| Phase-1 RCT-18 | 3 |
| Phase-1 RCT-49 | 3 |
| Phase-1 RCT-59 | 3 |
| Phase-1 RCT-72 | 3 |
| Phase-1 RCT-75 | 3 |
| Proliferating cell nuclear antigen gene | 3 |
| Sarcoplasmic reticulum calcium ATPase | 3 |
| Senescence marker protein-30 | 3 |
| 14-3-3 zeta | 2 |
| Acetyl-CoA carboxylase | 2 |
| Activating transcription factor 3 | 2 |
| C4b-binding protein | 2 |
| Carbonic anhydrase III | 2 |
| Cholesterol 7-alpha-hydroxylase (P450 VII) | 2 |
| Cytochrome P450 1A1 | 2 |
| DNA topoisomerase I | 2 |
| Ferritin H-chain | 2 |
| ID-1 | 2 |
| Iron-responsive element-binding protein | 2 |
| Macrophage inflammatory protein-1 alpha | 2 |
| Nucleosome assembly protein | 2 |
| Phase-1 RCT-110 | 2 |
| Phase-1 RCT-123 | 2 |
| Phase-1 RCT-15 | 2 |
| Phase-1 RCT-169 | 2 |
| Phase-1 RCT-177 | 2 |
| Phase-1 RCT-179 | 2 |
| Phase-1 RCT-182 | 2 |
| Phase-1 RCT-197 | 2 |
| Phase-1 RCT-214 | 2 |
| Phase-1 RCT-65 | 2 |
| Phase-1 RCT-71 | 2 |
| Phase-1 RCT-139 | 1 |
| 3-beta-hydroxysteroid dehydrogenase (HSD3B1) | 1 |
| 8-oxoguanine DNA glycosylase | 1 |
| Alcohol dehydrogenase 1 | 1 |
| AT-3 | 1 |
| Carnitine palmitoyl-CoA transferase | 1 |
| Caspase 6 | 1 |
| Choline kinase | 1 |
| Cyclin D3 | 1 |
| Cytochrome P450 2E1 | 1 |
| Elongation factor-1 alpha | 1 |
| H-rev107 | 1 |
| Insulin-like growth factor binding protein 5 | 1 |
| Matrix metalloproteinase-1 | 1 |
| Melanoma-associated antigen ME491 | 1 |
| MHC class I antigen RT1.A1(f) alpha-chain | 1 |
| Neuropeptide Y | 1 |
| Phase-1 RCT-109 | 1 |
| Protein O-mannosyltransferase 1 (Pomt1) | 1 |
| Phase-1 RCT-144 | 1 |
| Phase-1 RCT-191 | 1 |
| Phase-1 RCT-20 | 1 |
| Phase-1 RCT-204 | 1 |
| Phase-1 RCT-221 | 1 |
| Phase-1 RCT-225 | 1 |
| Phase-1 RCT-227 | 1 |
| Phase-1 RCT-248 | 1 |
| Phase-1 RCT-270 | 1 |
| Phase-1 RCT-277 | 1 |
| Phase-1 RCT-287 | 1 |
| Phase-1 RCT-289 | 1 |
| Phase-1 RCT-34 | 1 |
| Phase-1 RCT-40 | 1 |
| Phase-1 RCT-66 | 1 |
| Phase-1 RCT-70 | 1 |
| Phase-1 RCT-73 | 1 |
| Phase-1 RCT-87 | 1 |
| Preproalbumin | 1 |
| Protein kinase C alpha | 1 |
| Ribosomal protein L13A | 1 |
| Selenoprotein P | 1 |
| Tryptophan hydroxylase | 1 |

*Combination category is the number of training/test set gene list occurrences.

TABLE 21
Liver Toxicity Compound-Dose Prediction Values for 6 Hour
Data Predictive Genes (Combined List and Subsets)

| | Number | Prediction Measure* | | | |
|---|---|---|---|---|---|
| Gene Set | of Genes | Accuracy** | False Positive** | False Negative** | Geometric Mean** |
| Combo All | 98 | 0.712 (0.610-0.833) | 0.290 (0.100-0.431) | 0.317 (0.000-0.750) | 0.669 (0.474-0.804) |
| Combo 5 | 10 | 0.684 (0.597-0.756) | 0.329 (0.186-0.477) | 0.283 (0.000-0.750) | 0.663 (0.451-0.794) |
| Combo 4 | 7 | 0.667 (0.623-0.756) | 0.329 (0.200-0.431) | 0.375 (0.000-0.625) | 0.626 (0.545-0.754) |
| Combo 3 | 15 | 0.646 (0.534-0.704) | 0.363 (0.254-0.508) | 0.317 (0.083-0.500) | 0.648 (0.598-0.722) |
| Combo 2 | 24 | 0.684 (0.571-0.833) | 0.308 (0.100-0.462) | 0.400 (0.000-0.750) | 0.613 (0.474-0.744) |
| Combo 1 | 42 | 0.618 (0.494-0.846) | 0.367 (0.086-0.569) | 0.500 (0.167-0.750) | 0.526 (0.385-0.617) |

TABLE 22
Comparison of Predictivity for Correct Liver Toxicity Classification
and Random Classification Using Combo Gene Sets 6 h data.

| | | Accuracy | | Accuracy | |
|---|---|---|---|---|---|
| Gene | Gene | Correct Classification** | | Random Classification** | |
| List* | Subset* | Mean | Min-Max | Mean | Min.-Max. |
| Combo All | All Genes | 0.712 | (0.610-0.833) | 0.199 | (0.103-0.282) |
| Combo 5 | All Genes | 0.684 | (0.597-0.756) | 0.221 | (0.090-0.288) |
| Combo 4 | All Genes | 0.667 | (0.623-0.756) | 0.231 | (0.090-0.366) |
| Combo 3 | All Genes | 0.646 | (0.534-0.704) | 0.233 | (0.143-0.324) |
| Combo 2 | All Genes | 0.684 | (0.571-0.833) | 0.244 | (0.192-0.366) |
| Combo 1 | All Genes | 0.618 | (0.494-0.846) | 0.232 | (0.128-0.273) |
| *Combo Gene Lists as in Example 1, Table 1. For Combo lists all genes were used for | | | | | |
| prediction. | | | | | |
| **Accuracy = proportion of the total number of predictions that are correct. Non-calls are counted as incorrect | | | | | |
| # predictions. Accuracy was calculated for correct classifications of liver toxicity assigned to the samples and for | | | | | |
| # randomized classifications in the same proportions as the correct classifications. Values presented are the mean accuracy | | | | | |
| # values for 5 training/test sets with minimum and maximum accuracy values. | | | | | |

TABLE 23
Distribution of Compounds* in Individual Training and Test
Sets for 72 Hour Liver Data
Training and Test Set 1

| Training Set 1 | Training Set 1 | Test Set 1 | Test Set 1 |
|---|---|---|---|
| Negative | Positive | Negative | Positive |
| 5-FU | APAP | BUS | AFLB |
| AMPB | BRB | CIS | ANIT |
| AZA | CCL4 | CLOZ | DMN |
| BAP | LPS | CMC | |
| BEN | TET | DEX | |
| CAD | | DIF | |
| CAR | | ERY | |
| CHCL3 | | GAN | |
| CHEX | | HYD | |
| CHLOR | | PHEN | |
| CLO | | QUIN | |
| CPHOS | | STRZ | |
| CYCA | | TAM | |
| DOX | | THEO | |
| EST | | | |
| ETH | | | |
| GEN | | | |
| ISON | | | |
| KETO | | | |
| MET | | | |
| NAL | | | |
| PBARB | | | |
| PEG | | | |
| PUR | | | |

Training and Test Set 2

| Training Set 2 | Training Set 2 | Test Set 2 | Test Set 2 |
|---|---|---|---|
| Negative | Positive | Negative | Positive |
| 5-FU | AFLB | AMPB | APAP |
| BAP | ANIT | AZA | CCL4 |
| BUS | BRB | BEN | TET |
| CAR | DMN | CAD | |
| CHCL3 | LPS | CHLOR | |
| CHEX | | CLO | |
| CIS | | DEX | |
| CLOZ | | DOX | |
| CMC | | EST | |
| CPHOS | | ETH | |
| CYCA | | KETO | |
| DIF | | MET | |
| ERY | | NAL | |
| GAN | | PHEN | |
| GEN | | | |
| HYD | | | |
| ISON | | | |
| PBARB | | | |
| PEG | | | |
| PUR | | | |
| QUIN | | | |
| STRZ | | | |
| TAM | | | |
| THEO | | | |

Training and Test Set 3

| Training Set 3 | Training Set 3 | Test Set 3 | Test Set 3 |
|---|---|---|---|
| Negative | Positive | Negative | Positive |
| AMPB | APAP | 5-FU | AFLB |
| BAP | BRB | AZA | ANIT |
| BEN | CCL4 | CHEX | LPS |
| BUS | DMN | CLO | |
| CAD | TET | CMC | |
| CAR | | CPHOS | |
| CHCL3 | | CYCA | |
| CHLOR | | DIF | |
| CIS | | EST | |
| CLOZ | | HYD | |
| DEX | | MET | |
| DOX | | PEG | |
| ERY | | PUR | |
| ETH | | THEO | |
| GAN | | | |
| GEN | | | |
| ISON | | | |
| KETO | | | |
| NAL | | | |
| PBARB | | | |
| PHEN | | | |
| QUIN | | | |
| STRZ | | | |
| TAM | | | |

Training and Test Set 4

| Training Set 4 | Training Set 4 | Test Set 4 | Test Set 4 |
|---|---|---|---|
| Negative | Positive | Negative | Positive |
| 5-FU | ANIT | AMPB | AFLB |
| AZA | APAP | BUS | BRB |
| BAP | CCL4 | CAD | TET |
| BEN | DMN | CIS | |
| CAR | LPS | CLO | |
| CHCL3 | | CMC | |
| CHEX | | ETH | |
| CHLOR | | MET | |
| CLOZ | | NAL | |
| CPHOS | | PBARB | |
| CYCA | | PEG | |
| DEX | | PHEN | |
| DIF | | PUR | |
| DOX | | THEO | |
| ERY | | | |
| EST | | | |
| GAN | | | |
| GEN | | | |
| HYD | | | |
| ISON | | | |
| KETO | | | |
| QUIN | | | |
| STRZ | | | |
| TAM | | | |

Training and Test Set 5

| Training Set 5 | Training Set 5 | Test Set 5 | Test Set 5 |
|---|---|---|---|
| Negative | Positive | Negative | Positive |
| AZA | APAP | 5-FU | AFLB |
| BAP | BRB | AMPB | ANIT |
| BUS | CCL4 | BEN | LPS |
| CHEX | DMN | CAD | |
| CIS | TET | CAR | |
| CLO | | CHCL3 | |
| CLOZ | | CHLOR | |
| CMC | | CPHOS | |
| DEX | | CYCA | |
| DIF | | MET | |
| DOX | | PBARB | |
| ERY | | PEG | |
| EST | | PHEN | |
| ETH | | PUR | |
| GAN | | | |
| GEN | | | |
| HYD | | | |
| ISON | | | |
| KETO | | | |
| NAL | | | |
| QUIN | | | |
| STRZ | | | |
| TAM | | | |
| THEO | | | |

*For abbreviations please see Table 1 (Compound, Dose, Abbreviation, etc.)
**Negative = Compounds that did not elicit histopathology (score = 1)
Positive = Compounds that did elicit histopathology (score of 2 or greater)

TABLE 24
List of Genes, Whose Expression at 72 h Directly Correlates with
Liver Necrosis at 72 h, Ranked by Pearson Correlation Coefficient

| | Correlation |
|---|---|
| Gene | Coefficient |
| Osteoactivin | 0.7351 |
| Calpactin I heavy chain | 0.6821 |
| IgE binding protein | 0.6393 |
| Stathmin | 0.6238 |
| Melanoma-associated antigen ME491 | 0.6196 |
| Phase-1 RCT-68 | 0.6127 |
| High affinity IgE receptor gamma chain | 0.5971 |
| (FcERIgamma) | |
| Phase-1 RCT-121 | 0.5840 |
| Phase-1 RCT-179 | 0.5815 |
| Gamma-actin, cytoplasmic | 0.5770 |
| Phase-1 RCT-154 | 0.5761 |
| Thymosin beta-10 | 0.5760 |
| Alpha-tubulin | 0.5706 |
| 14-3-3 zeta | 0.5688 |
| Voltage-dependent anion channel (Vdac2) | 0.5651 |
| Phase-1 RCT-192 | 0.5593 |
| Phase-1 RCT-138 | 0.5574 |
| Uncoupling protein 2 | 0.5476 |
| Phase-1 RCT-24 | 0.5383 |
| Beta-actin | 0.5285 |
| 60S ribosomal protein L6 | 0.5232 |
| Phase-1 RCT-146 | 0.5016 |
| Collagen type II | 0.4978 |
| Cofilin | 0.4868 |
| Beta-tubulin, class I | 0.4827 |
| Pyruvate kinase, muscle | 0.4816 |
| Calpain 2 | 0.4808 |
| Annexin V | 0.4786 |
| Phase-1 RCT-144 | 0.4773 |
| Phase-1 RCT-207 | 0.4762 |
| Organic cation transporter 3 | 0.4760 |
| Phase-1 RCT-12 | 0.4744 |
| Tissue inhibitor of metalloproteinases-1 | 0.4729 |
| Beta-actin, sequence 2 | 0.4674 |
| Phase-1 RCT-293 | 0.4623 |
| Cyclin G | 0.4586 |
| Cathepsin S | 0.4472 |
| Multidrug resistant protein-2 | 0.4446 |
| Phase-1 RCT-211 | 0.4420 |
| Multidrug resistant protein-1 | 0.4402 |
| Cyclin D1 | 0.4382 |
| Nucleoside diphosphate kinase beta isoform | 0.4331 |
| Biliverdin reductase | 0.4310 |
| 60S ribosomal protein L6 (alternate clone 1) | 0.4308 |
| Phase-1 RCT-215 | 0.4231 |
| Cathepsin B | 0.4180 |
| Phase-1 RCT-37 | 0.4077 |
| Ribosomal protein S8 | 0.4072 |
| Ribosomal protein S9 | 0.4040 |
| Heme oxygenase | 0.4033 |
| CD44 metastasis suppressor gene | 0.4021 |

TABLE 25
List of Genes, Whose Expression at 72 h Inversely
Correlates with Liver Necrosis at 72 h, Ranked
by Spearman Correlation Coefficient

| | Correlation |
|---|---|
| Gene | Coefficient |
| Phase-1 RCT-123 | −0.2509 |
| Phase-1 RCT-185 | −0.2516 |
| Cholesterol esterase | −0.2518 |
| C-reactive protein | −0.2518 |
| Phase-1 RCT-260 | −0.2536 |
| Retinol dehydrogenase type III | −0.2543 |
| Phase-1 RCT-67 | −0.2561 |
| Aquaporin-3 (AQP3) | −0.2565 |
| NADH-cytochrome b5 reductase | −0.2585 |
| Phase-1 RCT-278 | −0.2604 |
| Interferon inducible protein 10 | −0.2662 |
| Acetylcholine receptor epsilon | −0.2675 |
| CDK108 | −0.2676 |
| Phase-1 RCT-219 | −0.2683 |
| Phase-1 RCT-73 | −0.2685 |
| Phase-1 RCT-29 | −0.2707 |
| Gap junction membrane channel protein beta 1 (Gjb1) | −0.2731 |
| Phase-1 RCT-285 | −0.2735 |
| Phase-1 RCT-38 | −0.2746 |
| Cytochrome P450 2D18 | −0.2768 |
| Phase-1 RCT-227 | −0.2774 |
| Matrin F/G | −0.2781 |
| Phase-1 RCT-33 | −0.2809 |
| Phase-1 RCT-280 | −0.2818 |
| Equilbrative nitrobenzylthioinosine-sensitive nucleoside | −0.2827 |
| transporter | |
| L-gulono-gamma-lactone oxidase | −0.2837 |
| Aryl sulfotransferase | −0.2838 |
| alpha-1,2-fucosyltransferase | −0.2848 |
| Phase-1 RCT-98 | −0.2853 |
| Urinary protein 2 precursor | −0.2874 |
| Tyrosine hydroxylase | −0.2897 |
| Cytochrome P450 3A1 | −0.2910 |
| NIPK | −0.2926 |
| Protein tyrosine phosphatase, receptor type, D | −0.2952 |
| Contrapsin-like protease inhibitor (CPi-21) | −0.2961 |
| Phase-1 RCT-187 | −0.2963 |
| Connexin-32 | −0.2995 |
| Phase-1 RCT-81 | −0.2999 |
| Phase-1 RCT-256 | −0.3038 |
| Cytochrome P450 2A3 | −0.3078 |
| Insulin-like growth factor I | −0.3079 |
| Apolipoprotein CIII | −0.3097 |
| Phase-1 RCT-292 | −0.3099 |
| Phase-1 RCT-178 | −0.3122 |
| Phase-1 RCT-102 | −0.3187 |
| Arginosuccinate synthetase 1 | −0.3193 |
| Fatty acid synthase | −0.3234 |
| Aldehyde dehydrogenase 2 | −0.3355 |
| N-hydroxy-2-acetylaminofluorene sulfotransferase (ST1C1) | −0.3355 |
| Phase-1 RCT-48 | −0.3428 |
| Phase-1 RCT-149 | −0.3456 |
| Phase-1 RCT-117 | −0.3466 |
| JNK1 stress activated protein kinase | −0.3517 |
| Phase-1 RCT-36 | −0.3552 |
| Phase-1 RCT-78 | −0.3568 |
| Phase-1 RCT-164 | −0.3596 |
| Stearyl-CoA desaturase, liver | −0.3666 |
| Glycine methyltransferase | −0.3758 |
| Dynamin-1 (D100) | −0.3774 |
| Betaine homocysteine methyltransferase (BHMT) | −0.3779 |
| Phase-1 RCT-107 | −0.3869 |
| Cytochrome P450 2C11 | −0.3876 |
| Phase-1 RCT-290 | −0.4002 |
| Apolipoprotein AII | −0.4022 |
| Insulin-like growth factor I, exon 6 | −0.4110 |
| Alpha-2-microglobulin | −0.4294 |

TABLE 26
List of genes whose expression at 72 hours
is predictive of liver toxicity at 72 hours

| | Combination |
|---|---|
| Gene Name | Category |
| Calpactin I heavy chain | 5 |
| Osteoactivin | 5 |
| 60S ribosomal protein L6 | 4 |
| Collagen type II | 4 |
| Gamma-actin, cytoplasmic | 4 |
| Glycine methyltransferase | 4 |
| High affinity IgE receptor gamma chain (FcERIgamma) | 4 |
| IgE binding protein | 4 |
| Phase-1 RCT-179 | 4 |
| Phase-1 RCT-192 | 4 |
| Stathmin | 4 |
| Thymosin beta-10 | 4 |
| Uncoupling protein 2 | 4 |
| Alpha-2-microglobulin | 3 |
| Alpha-tubulin | 3 |
| Biliverdin reductase | 3 |
| Cofilin | 3 |
| Heme oxygenase | 3 |
| Melanoma-associated antigen ME491 | 3 |
| Multidrug resistant protein-2 | 3 |
| Phase-1 RCT-121 | 3 |
| Phase-1 RCT-138 | 3 |
| Phase-1 RCT-146 | 3 |
| Voltage-dependent anion channel (Vdac2) | 3 |
| Phase-1 RCT-39 | 3 |
| Phase-1 RCT-68 | 3 |
| Ribosomal protein S9 | 3 |
| 14-3-3 zeta | 2 |
| Adenine nucleotide translocator 1 | 2 |
| Alpha-2-macroglobulin, sequence 2 | 2 |
| Annexin V | 2 |
| Beta-actin | 2 |
| Beta-actin, sequence 2 | 2 |
| Beta-tubulin, class I | 2 |
| Calpain 2 | 2 |
| Cyclin D1 | 2 |
| Cystatin C | 2 |
| Cytochrome P450 2C11 | 2 |
| Glutathione S-transferase theta-1 | 2 |
| Insulin-like growth factor I, exon 6 | 2 |
| Multidrug resistant protein-1 | 2 |
| Nucleoside diphosphate kinase beta isoform | 2 |
| Organic cation transporter 3 | 2 |
| Phase-1 RCT-107 | 2 |
| Phase-1 RCT-12 | 2 |
| Phase-1 RCT-144 | 2 |
| Phase-1 RCT-154 | 2 |
| Phase-1 RCT-207 | 2 |
| Phase-1 RCT-211 | 2 |
| Phase-1 RCT-215 | 2 |
| Phase-1 RCT-24 | 2 |
| Phase-1 RCT-78 | 2 |
| Phase-1 RCT-81 | 2 |
| 60S ribosomal protein L6 (alternate clone 1) | 1 |
| Aldehyde dehydrogenase 2 | 1 |
| Alpha-1 microglobulin/bikunin precursor (Ambp) | 1 |
| Alpha-prothymosin | 1 |
| Apolipoprotein AII | 1 |
| Apolipoprotein C1 | 1 |
| Apolipoprotein CIII | 1 |
| Arginosuccinate synthetase 1 | 1 |
| Urinary protein 2 precursor | 1 |
| Betaine homocysteine methyltransferase (BHMT) | 1 |
| Cathepsin B | 1 |
| Cathepsin S | 1 |
| Cholesterol esterase | 1 |
| Connexin-32 | 1 |
| Contrapsin-like protease inhibitor (CPi-21) | 1 |
| C-reactive protein | 1 |
| Cyclin G | 1 |
| Cytochrome P450 2C23 | 1 |
| Cytochrome P450 2D18 | 1 |
| Dynamin-1 (D100) | 1 |
| Equilbrative nitrobenzylthioinosine-sensitive nucleoside | 1 |
| transporter | |
| Fatty acid synthase | 1 |
| Gap junction membrane channel protein beta 1 (Gjb1) | 1 |
| Hypoxanthine-guanine phosphoribosyltransferase | 1 |
| Insulin-like growth factor I | 1 |
| Interleukin-18 | 1 |
| JNK1 stress activated protein kinase | 1 |
| Lecithin: cholesterol acyltransferase | 1 |
| L-gulono-gamma-lactone oxidase | 1 |
| Matrin F/G | 1 |
| NADH-cytochrome b5 reductase | 1 |
| N-hydroxy-2-acetylaminofluorene sulfotransferase (ST1C1) | 1 |
| p53 | 1 |
| p55CDC | 1 |
| Phase-1 RCT-102 | 1 |
| Phase-1 RCT-109 | 1 |
| Phase-1 RCT-145 | 1 |
| Phase-1 RCT-149 | 1 |
| Phase-1 RCT-164 | 1 |
| Phase-1 RCT-173 | 1 |
| Phase-1 RCT-185 | 1 |
| Phase-1 RCT-187 | 1 |
| Phase-1 RCT-219 | 1 |
| Phase-1 RCT-227 | 1 |
| Phase-1 RCT-230 | 1 |
| Phase-1 RCT-256 | 1 |
| Phase-1 RCT-278 | 1 |
| Phase-1 RCT-285 | 1 |
| Phase-1 RCT-290 | 1 |
| Phase-1 RCT-292 | 1 |
| Phase-1 RCT-293 | 1 |
| Phase-1 RCT-33 | 1 |
| Phase-1 RCT-36 | 1 |
| Phase-1 RCT-37 | 1 |
| Phase-1 RCT-38 | 1 |
| Phase-1 RCT-48 | 1 |
| Phase-1 RCT-58 | 1 |
| Phase-1 RCT-61 | 1 |
| Proliferating cell nuclear antigen gene | 1 |
| Protein tyrosine phosphatase, receptor type, D | 1 |
| PTEN/MMAC1 | 1 |
| Pyruvate kinase, muscle | 1 |
| Retinol dehydrogenase type III | 1 |
| Ribosomal protein S8 | 1 |
| Stearyl CoA desaturase, liver | 1 |
| Thymidylate synthase | 1 |
| Ubiquitin conjugating enzyme (RAD 6 homologue) | 1 |
| Zinc finger protein | 1 |

* Combination category is the number of training/test set gene list occurrences.

TABLE 27
Liver Toxicity Compound-Dose Prediction Values for 72
Hour Data Predictive Genes (Combined List and Subsets)

| | | Prediction Measure * ** | | | |
|---|---|---|---|---|---|
| | | | False | False | |
| | | | Positive | Negative | Geometric |
| Gene List | | Accuracy | Rate | Rate | Mean 2 |
| Combo All | Mean | 0.790 | 0.192 | 0.342 | 0.729 |
| | Minimum | 0.690 | 0.134 | 0.250 | 0.642 |
| | Maximum | 0.835 | 0.293 | 0.417 | 0.775 |
| Combo 5 | Mean | 0.641 | 0.351 | 0.417 | 0.615 |
| | Minimum | 0.523 | 0.209 | 0.333 | 0.513 |
| | Maximum | 0.772 | 0.474 | 0.500 | 0.726 |
| Combo 4 | Mean | 0.749 | 0.226 | 0.417 | 0.664 |
| | Minimum | 0.652 | 0.147 | 0.333 | 0.533 |
| | Maximum | 0.823 | 0.350 | 0.667 | 0.753 |
| Combo 3 | Mean | 0.715 | 0.269 | 0.400 | 0.660 |
| | Minimum | 0.699 | 0.244 | 0.333 | 0.558 |
| | Maximum | 0.747 | 0.293 | 0.583 | 0.710 |
| Combo 2 | Mean | 0.713 | 0.261 | 0.500 | 0.602 |
| | Minimum | 0.644 | 0.192 | 0.333 | 0.524 |
| | Maximum | 0.767 | 0.320 | 0.625 | 0.710 |
| Combo 1 | Mean | 0.570 | 0.449 | 0.275 | 0.631 |
| | Minimum | 0.529 | 0.403 | 0.125 | 0.551 |
| | Maximum | 0.620 | 0.480 | 0.417 | 0.692 |
| * Prediction measures are given as means and range of values for five training/test sets using 72 hour array data and | | | | | |
| # gene lists as presented in Example 5. Unit of prediction was the animal and the predictive classification was for liver | | | | | |
| # necrosis observed at 72 hours after treatment. | | | | | |
| ** Standard prediction measures were used as defined in Materials and Methods. As described in Materials and Methods | | | | | |
| # In these analyses cases where no prediction was made because the p-value ratio exceeded the cutoff-value (generally 0.5) | | | | | |
| # the non-call was considered to be incorrect. | | | | | |

TABLE 28
Comparison of Predictivity for Correct Liver Toxicity Classification
and Random Classification Using Combo Gene Sets 72 h data

Accuracy ***

| | | Correct | Random |
|---|---|---|---|
| Gene List | | Classification | Classification |
| Combo All | Mean | 0.790 | 0.229 |
| | Minimum | 0.690 | 0.035 |
| | Maximum | 0.835 | 0.316 |
| Combo 5 | Mean | 0.641 | 0.263 |
| | Minimum | 0.523 | 0.124 |
| | Maximum | 0.772 | 0.418 |
| Combo 4 | Mean | 0.749 | 0.257 |
| | Minimum | 0.652 | 0.186 |
| | Maximum | 0.823 | 0.391 |
| Combo 3 | Mean | 0.715 | 0.281 |
| | Minimum | 0.699 | 0.161 |
| | Maximum | 0.747 | 0.367 |
| Combo 2 | Mean | 0.713 | 0.211 |
| | Minimum | 0.644 | 0.115 |
| | Maximum | 0.767 | 0.304 |
| Combo 1 | Mean | 0.570 | 0.235 |
| | Minimum | 0.529 | 0.023 |
| | Maximum | 0.620 | 0.354 |

* Combo Gene Lists as in Example 1, Table 1. For Combo lists all genes were used for prediction.
** Accuracy = proportion of the total number of predictions that are correct. Non-calls are counted as incorrect predictions. Accuracy was calculated for correct classifications of liver toxicity assigned to the samples and for randomized classifications in the same proportions as the correct classifications. Values presented are the mean accuracy values for 5 training/test sets with minimum and maximum accuracy values.

TABLE 29
Prediction of Liver Toxicity for Samples External to Database

| | | | Prediction Values** | | | | | |
|---|---|---|---|---|---|---|---|---|
| Predicting | | | | P-Value | No | No P- | Yes | Yes P |
| Gene Set* | Treatment | Animal | Prediction | Ratio | Votes | Value | Votes | Value |
| Combo 6 | Cephaloridine 1500 mg/kg i.p. 24 h | 501 | yes | 0.000 | 0 | 1 | 10 | 0 |
| Combo 6 | Cephaloridine 1500 mg/kg i.p. 24 h | 506 | yes | 0.000 | 0 | 1 | 10 | 0 |
| Combo 6 | Cephaloridine 1500 mg/kg i.p. 24 h | 508 | yes | 0.000 | 0 | 1 | 10 | 0 |
| Combo 6 | Cisplatin 20 mg/kg i.p. 24 h | 602 | yes | 0.000 | 2 | 1 | 8 | 0 |
| Combo 6 | Cisplatin 20 mg/kg i.p. 24 h | 603 | yes | 0.000 | 0 | 1 | 10 | 0 |
| Combo 6 | Cisplatin 20 mg/kg i.p. 24 h | 604 | yes | 0.000 | 0 | 1 | 10 | 0 |
| Combo 5 | Cephaloridine 1500 mg/kg i.p. 24 h | 501 | yes | 0.001 | 4 | 1 | 6 | 0.001 |
| Combo 5 | Cephaloridine 1500 mg/kg i.p. 24 h | 506 | yes | 0.000 | 1 | 1 | 9 | 0 |
| Combo 5 | Cephaloridine 1500 mg/kg i.p. 24 h | 508 | yes | 0.000 | 2 | 1 | 8 | 0 |
| Combo 5 | Cisplatin 20 mg/kg i.p. 24 h | 602 | yes | 0.208 | 7 | 0.945 | 3 | 0.197 |
| Combo 5 | Cisplatin 20 mg/kg i.p. 24 h | 603 | yes | 0.208 | 7 | 0.945 | 3 | 0.197 |
| Combo 5 | Cisplatin 20 mg/kg i.p. 24 h | 604 | yes | 0.001 | 4 | 1 | 6 | 0.001 |
| Combo 4 | Cephaloridine 1500 mg/kg i.p. 24 h | 501 | yes | 0.000 | 1 | 1 | 9 | 0 |
| Combo 4 | Cephaloridine 1500 mg/kg i.p. 24 h | 506 | yes | 0.000 | 2 | 1 | 8 | 0 |
| Combo 4 | Cephaloridine 1500 mg/kg i.p. 24 h | 508 | yes | 0.000 | 0 | 1 | 10 | 0 |
| Combo 4 | Cisplatin 20 mg/kg i.p. 24 h | 602 | yes | 0.010 | 5 | 0.999 | 5 | 0.01 |
| Combo 4 | Cisplatin 20 mg/kg i.p. 24 h | 603 | yes | 0.000 | 1 | 1 | 9 | 0 |
| Combo 4 | Cisplatin 20 mg/kg i.p. 24 h | 604 | yes | 0.000 | 1 | 1 | 9 | 0 |
| Combo 3 | Cephaloridine 1500 mg/kg i.p. 24 h | 501 | yes | 0.001 | 4 | 1 | 6 | 0.001 |
| Combo 3 | Cephaloridine 1500 mg/kg i.p. 24 h | 506 | yes | 0.208 | 7 | 0.945 | 3 | 0.197 |
| Combo 3 | Cephaloridine 1500 mg/kg i.p. 24 h | 508 | | 0.606 | 8 | 0.803 | 2 | 0.487 |
| Combo 3 | Cisplatin 20 mg/kg i.p. 24 h | 602 | yes | 0.208 | 7 | 0.945 | 3 | 0.197 |
| Combo 3 | Cisplatin 20 mg/kg i.p. 24 h | 603 | yes | 0.001 | 4 | 1 | 6 | 0.001 |
| Combo 3 | Cisplatin 20 mg/kg i.p. 24 h | 604 | yes | 0.055 | 6 | 0.99 | 4 | 0.055 |
| Combo 2 | Cephaloridine 1500 mg/kg i.p. 24 h | 501 | yes | 0.000 | 3 | 1 | 7 | 0 |
| Combo 2 | Cephaloridine 1500 mg/kg i.p. 24 h | 506 | yes | 0.000 | 3 | 1 | 7 | 0 |
| Combo 2 | Cephaloridine 1500 mg/kg i.p. 24 h | 508 | yes | 0.000 | 3 | 1 | 7 | 0 |
| Combo 2 | Cisplatin 20 mg/kg i.p. 24 h | 602 | yes | 0.010 | 5 | 0.999 | 5 | 0.01 |
| Combo 2 | Cisplatin 20 mg/kg i.p. 24 h | 603 | yes | 0.000 | 3 | 1 | 7 | 0 |
| Combo 2 | Cisplatin 20 mg/kg i.p. 24 h | 604 | yes | 0.000 | 2 | 1 | 8 | 0 |
| Combo 1 | Cephaloridine 1500 mg/kg i.p. 24 h | 501 | yes | 0.000 | 1 | 1 | 9 | 0 |
| Combo 1 | Cephaloridine 1500 mg/kg i.p. 24 h | 506 | yes | 0.000 | 1 | 1 | 9 | 0 |
| Combo 1 | Cephaloridine 1500 mg/kg i.p. 24 h | 508 | yes | 0.000 | 3 | 1 | 7 | 0 |
| Combo 1 | Cisplatin 20 mg/kg i.p. 24 h | 602 | yes | 0.001 | 4 | 1 | 6 | 0.001 |
| Combo 1 | Cisplatin 20 mg/kg i.p. 24 h | 603 | yes | 0.000 | 3 | 1 | 7 | 0 |
| Combo 1 | Cisplatin 20 mg/kg i.p. 24 h | 604 | yes | 0.000 | 3 | 1 | 7 | 0 |

*All genes used for Combo Gene Lists as in Example 1, Table 1.
**Prediction values are output from prediction program. Values include prediction
(yes = liver toxicity predicted, no = no liver toxicity predicted), numbers of yes and no votes
from 10 nearest neighbors, the p-value for the no and yes votes and the p-value ratio for the
predicted class over the not predicted class. A p-value ratio cutoff of 0.5 was used

TABLE 30
K-means Cluster Analysis of Combo 5, 4, 3 and 2 Gene Set

Cluster 1
Gamma-actin, cytoplasmic
Insulin-like growth factor binding protein 1
RCT-68
RCT-39
Integrin betal
Zinc finger protein
RCT-50
c-myc
RCT-144
PAR interacting protein
RCT-145
RCT-49
RCT-213
RCT-258
RCT-241
Gadd45
Heme oxygenase
14-3-3 zeta
Beta-actin
Ornithine decarboxylase
RCT-207
Bax (alpha)
ID-1
RCT-180
RCT-191
Cluster 2
Senescence marker protein-30
RCT-33
RCT-36
RCT-139
Cluster 3
RCT-78
L-gulono-gamma-lactone oxidase
RCT-256
RCT-38
RCT-296
RCT-92
Dynamin-1 (D100)
RCT-128
RCT-264
RCT-89
RCT-270
RCT-182
RCT-291
Hepatic lipase
RCT-271
Matrin F/G
RCT-288
RCT-189
Cluster 4
N-hydroxy-2-acetylaminofluorene
sulfotransferase (ST1C1)
Equilbrative nitrobenzylthioinosine-sensitive
nucleoside transporter
Paraoxonase 1
Insulin-like growth factor binding protein 3
RCT-83
Epidermal growth factor
Cluster 5
Phase-1 RCT-40
3-hydroxyisobutyrate dehydrogenase
Cluster 6
Alpha 1 - inhibitor III
RCT-48
RCT-102
RCT-117
Cluster 7
Cathepsin L, sequence 2
RCT-179
Ribosomal protein S17
RCT-152
60S ribosomal protein L6
Voltage-dependent anion channel 2 (Vdac2)
Cluster 8
Carbonic anhydrase III
Organic anion transporter 3
RCT-123

TABLE 31
RCT genes (ESTs) Predictive for Liver Necrosis at 72 hours: Best Homology Matches

| Gene Name | Homology |
|---|---|
| RCT-102 | Mouse pentylenetetrazol-related mRNA PTZ-17 (3′UTR of E3.1) |
| RCT-107 | no significant homology found |
| RCT-109 | Rattus norvegicus nesprin-1 mRNA |
| RCT-110 | Homo Sapiens, clone IMAGE: 3677434, mRNA |
| RCT-117 | no significant homology found |
| RCT-12 | no significant homology found |
| RCT-121 | no significant homology found |
| RCT-123 | no significant homology found |
| RCT-127 | no significant homology found |
| RCT-128 | Mus Musculus angiopoietin-related protein 3 (Angptl3) |
| RCT-137 | Mus Musculus adult male tongue cDNA |
| RCT-138 | Mus Musculus DAP10 (Dap10) gene |
| RCT-139 | no significant homology found |
| RCT-144 | Mus Musculus, similar to nucleolar protein (KKE/D repeat), clone |
| | IMAGE: 3491448, mRNA, partial cds. |
| RCT-145 | Mus Musculus 10 day old male pancreas cDNA, RIKEN full-length enriched |
| | library, clone: 1810014B19, full insert sequence |
| RCT-146 | Mus Musculus 8 days embryo cDNA, RIKEN full-length enriched library, |
| | clone: 5730458E20 |
| RCT-149 | Mouse mRNA fragment for serum amyloid A (SAA) 3 protein |
| RCT-15 | Mus Musculus ubiquitin conjugating enzyme 7 mRNA, complete cds |
| RCT-152 | Mus Musculus, eukaryotic translation elongation factor 1 beta 2, clone |
| | MGC: 6763 IMAGE: 3600850, mRNA, complete cds. |
| RCT-154 | Mus Musculus vacuolar ATPase subunit D (Atp6m) mRNA, complete cds |
| RCT-162 | Mus Musculus, clone IMAGE: 3501507 |
| RCT-164 | Mus Musculus adult male testis cDNA, RIKEN full-length enriched library, |
| | clone: 4932443D16 |
| RCT-168 | M.musculus mRNA for low density lipoprotein receptor, ACCESSION X64414 |
| | S51850 |
| RCT-169 | Mus Musculus, small inducible cytokine B subfamily (Cys-X-Cys), member 9, |
| | clone MGC: 6179 IMAGE: 3257716, mRNA, complete |
| RCT-173 | Mus Musculus NADP +− specific isocitrate dehydrogenase mRNA, complete |
| | cds; nuclear gene for mitochondrial product |
| RCT-177 | Mus Musculus, Similar to peroxisomal deltas, delta2-enoyl-Coenzyme A |
| | isomerase, clone MGC: 5644 IMAGE: 3591615 |
| RCT-179 | Rat nucleolar protein B23.2 mRNA |
| RCT-18 | no significant homology found |
| RCT-180 | Mus Musculus B-cell receptor-associated protein 37 (Bcap37 |
| RCT-181 | Mus Musculus adult male testis cDNA |
| RCT-182 | Rattus norvegicus gib mRNA for diacetyl/L-xylulose reductase |
| RCT-185 | no significant homology found |
| RCT-187 | Mus Musculus 11 days pregnant adult female ovary and uterus cDNA, RIKEN |
| | full-length enriched library, clone: 5033416F05, full insert sequence |
| RCT-189 | Rattus norvegicus eukaryotic translation initiation factor 4E (Eif4e), mRNA |
| RCT-191 | Mus Musculus, Similar to proteasome (prosome, macropain) 26S subunit, |
| | non-ATPase, 3, clone MGC: 6405 IMAGE: 3586427, mRNA, complete cds |
| RCT-192 | Mus Musculus 18 days embryo cDNA, RIKEN full-length enriched library, |
| | clone: 1110033J19 |
| RCT-197 | Rattus norvegicus Protein kinase, interferon-inducible double stranded RNA |
| | dependent (Prkr), mRNA |
| RCT-20 | Mus Musculus cysteine and histidine-rich domain (CHORD)-containing, |
| RCT-204 | Mouse DNA sequence from clone RP23-138F20 on chromosome 13, |
| | complete sequence [Mus Musculus] |
| RCT-205 | no significant homology found |
| RCT-207 | Mus Musculus Ran binding protein 5 mRNA, partial cds |
| RCT-211 | Mus Musculus adult male kidney cDNA, RIKEN full-length enriched library, |
| | clone: 0610009C22 |
| RCT-213 | Homo Sapiens pM5 protein (PM5), mRNA |
| RCT-214 | Mus Musculus putative NAD(P)H steroid dehydrogenase mRNA |
| RCT-215 | Mus Musculus RAB/Rip protein mRNA |
| RCT-219 | Rattus norvegicus 2′5′ oligoadenylate synthetase-2 mRNA, complete cds |
| RCT-221 | no significant homology found |
| RCT-225 | Rattus norvegicus chromosome 4 clone RP31-327J16 strain Brown Norway, |
| | complete sequence |
| RCT-227 | no significant homology found |
| RCT-230 | Mus Musculus GDP-dissociation inhibitor mRNA, preferentially expressed in |
| | hematopoietic cells, complete cds |
| RCT-239 | Mus Musculus adult male tongue cDNA, RIKEN full-length enriched library, |
| | clone: 2300007B01, full insert sequence |
| RCT-24 | Mus Musculus, tubulin alpha 8, clone MGC: 28850 IMAGE: 4507364, mRNA, |
| RCT-241 | Mus Musculus oncostatin receptor (Osmr), mRNA |
| RCT-242 | Rattus norvegicus B-cell translocation gene 2, anti-proliferative (Btg2), |
| RCT-248 | Mus Musculus claudin 18 (Cldn18-pending), mRNA |
| RCT-252 | Mus Musculus EH-domain containing 3 (Ehd3), |
| RCT-256 | Mus Musculus, Similar to betaine-homocysteine methyltransferase 2, clone |
| | MGC: 19186 IMAGE: 4235455 |
| RCT-258 | Mus Musculus, clone MGC: 6139 IMAGE: 3487295, mRNA |
| RCT-264 | Mus Musculus sodium-sulfate cotransporter (Nas1) gene |
| RCT-270 | Mus Musculus, RIKEN cDNA 2010011120 gene, clone MGC: 27703, |
| | IMAGE: 4924329, mRNA, complete cds |
| RCT-271 | Homlogous to Mus Musculus, clone MGC: 27581 IMAGE: 4489072, mRNA |
| RCT-277 | no significant homology found |
| RCT-278 | Mus Musculus brain protein 17 (Brp17), mRNA |
| RCT-285 | Mus Musculus, Similar to single Ig IL-1 R-related protein, clone MGC: 18899 |
| | IMAGE: 4240425, mRNA, complete cds |
| RCT-287 | Mus Musculus adult male kidney cDNA clone: 0610010120 |
| RCT-288 | no significant homology found |
| RCT-289 | Mus Musculus adult male liver cDNA, RIKEN full-length enriched library, |
| | clone: 1300003K24, full insert sequence |
| RCT-290 | Homo Sapiens chromosome 14 clone BAC 201F1 map 14q24.3, complete |
| | sequence |
| RCT-291 | no significant homology found |
| RCT-292 | Rattus norvegicus 2′5′ oligoadenylate synthetase-2 |
| RCT-293 | Mus Musculus 18 days embryo cDNA, RIKEN full-length enriched library, |
| | clone: 1110021C22 |
| RCT-296 | Mus Musculus corticosteroid binding globulin (Cbg) |
| RCT-33 | no significant homology found |
| RCT-34 | no significant homology found |
| RCT-36 | no significant homology found |
| RCT-37 | no significant homology found |
| RCT-38 | Mus Musculus betaine-homocysteine methyltransferase 2 (Bhmt2) mRNA, |
| RCT-39 | no significant homology found |
| RCT-40 | Rattus norvegicus Cathepsin C (dipeptidyl peptidase I) (Ctsc) |
| RCT-48 | Mus Musculus adult male liver cDNA, RIKEN full-length enriched library, |
| | clone: 1300003K24, full insert sequence |
| RCT-49 | No match with score above 200 |
| RCT-50 | Mus Musculus fibroblast growth factor regulated protein 2 |
| RCT-55 | M. musculus myoglobin gene exons 2-3 |
| RCT-58 | Rat mRNA for delta-4-3-ketosteroid 5-beta-reductase, complete cds |
| RCT-59 | no significant homology found |
| RCT-61 | no significant homology found |
| RCT-65 | no significant homology found |
| RCT-66 | M. musculus mRNA for low density lipoprotein receptor |
| RCT-68 | Rattus norvegicus nucleosome assembly protein mRNA |
| RCT-70 | Mus Musculus adult male testis cDNA, RIKEN full-length enriched library, |
| | clone: 4933406P04, full insert sequence |
| RCT-71 | Mus Musculus, clone MGC: 11987 IMAGE: 3601737, mRNA |
| RCT-72 | no significant homology found |
| RCT-73 | no significant homology found |
| RCT-75 | Mus Musculus adult male liver cDNA, RIKEN full-length enriched library, |
| | clone: 1300002K09, full insert sequence |
| RCT-78 | Mus Musculus adult male lung cDNA, RIKEN full-length enriched library, |
| | clone: 1200015G06, full insert sequence |
| RCT-81 | no significant homology found |
| RCT-82 | Mus Musculus nucleosome binding protein 1 (Nsbp1), |
| RCT-83 | no significant homology found |
| RCT-87 | Mus Musculus adult male tongue cDNA |
| RCT-88 | no significant homology found |
| RCT-89 | no significant homology found |
| RCT-92 | no significant homology found |

TABLE 33
Liver Hepatocellular Necrosis Predictive Genes
Whose Protein Products Are Known to be Secreted

Apolipoprotein AII
Apolipoprptein C1
Apolipoprotein CIII
C4b-binding protein
C-reactive protein
Cystatin C
Epidermal growth factor
Ferritin H-chain
Insulin-like growth factor I
Insulin-like growth factor I, exon 6
Interleukin-18
Lecithin: cholesterol acyltransferase
Macrophage inflammatory protein-1 alpha
Macrophage inflammatory protein-2 alpha
Matrix metalloproteinase-1
NGF-inducible anti-proliferative putative secreted protein (PC3)
Selenoprotein P
T-cell cyclophilin
Transthyretin

TABLE 37
Predictive Performance of Predictive Genes Organized by Occurrence
on Training/Test Set Lists (Combo number) and Time Point

| | | Number | | |
|---|---|---|---|---|
| Time | | of | | |
| Point | Gene Set | Genes | Accuracy** | Geometric Mean** |
| 24 h | Combo All | 142 | 0.924 (0.872-0.960) | 0.917 (0.772-0.956) |
| 24 h | Combo 5 | 3 | 0.896 (0.837-0.961) | 0.901 (0.868-0.941) |
| 24 h | Combo 4 | 7 | 0.857 (0.796-0.913) | 0.862 (0.800-0.915) |
| 24 h | Combo 3 | 22 | 0.865 (0.755-0.923) | 0.900 (0.854-0.954) |
| 24 h | Combo 2 | 36 | 0.912 (0.851-0.950) | 0.904 (0.762-0.955) |
| 24 h | Combo 1 | 74 | 0.894 (0.853-0.941) | 0.844 (0.705-0.949) |
| 6 h | Combo All | 98 | 0.712 (0.610-0.833) | 0.669 (0.474-0.804) |
| 6 h | Combo 5 | 10 | 0.684 (0.597-0.756) | 0.663 (0.451-0.794) |
| 6 h | Combo 4 | 7 | 0.667 (0.623-0.756) | 0.626 (0.545-0.754) |
| 6 h | Combo 3 | 15 | 0.646 (0.534-0.704) | 0.648 (0.598-0.722) |
| 6 h | Combo 2 | 24 | 0.684 (0.571-0.833) | 0.613 (0.474-0.744) |
| 6 h | Combo 1 | 42 | 0.618 (0.494-0.846) | 0.526 (0.385-0.617) |
| 72 h | Combo All | 130 | 0.790 (0.690-0.835) | 0.729 (0.642-0.775) |
| 72 h | Combo 5 | 1 | 0.641 (0.523-0.772) | 0.615 (0.513-0.726) |
| 72 h | Combo 4 | 17 | 0.749 (0.652-0.823) | 0.664 (0.533-0.753) |
| 72 h | Combo 3 | 21 | 0.715 (0.699-0.747) | 0.660 (0.558-0.710) |
| 72 h | Combo 2 | 23 | 0.713 (0.644-0.767) | 0.602 (0.524-0.710) |
| 72 h | Combo 1 | 68 | 0.570 (0.529-0.620) | 0.631 (0.551-0.692) |

TABLE 38
266 Liver Toxicity Predictive Genes Organized by Time Point and Combo Class

| Gene | 6 h | 24 h | 72 h |
|---|---|---|---|
| {Ribosomal protein L6} | Not Found | Combo 1 | Combo 1 |
| 14-3-3 zeta | Combo 2 | Combo 3 | Combo 2 |
| 17-beta hydroxysteroid dehydrogenase, type 2 | Combo 1 | Combo 2 | Not Found |
| 25-DX | Not Found | Combo 1 | Not Found |
| 3-beta-hydroxysteroid dehydrogenase (HSD3B1) | Combo 1 | Not Found | Not Found |
| 3-hydroxyisobutyrate dehydrogenase | Not Found | Combo 2 | Not Found |
| 60S ribosomal protein L6 | Not Found | Combo 2 | Combo 4 |
| 8-oxoguanine DNA glycosylase | Combo 1 | Not Found | Not Found |
| Acetyl-CoA carboxylase | Combo 2 | Not Found | Not Found |
| Activating transcription factor 3 | Combo 2 | Not Found | Not Found |
| Adenine nucleotide translocator 1 | Not Found | Not Found | Combo 2 |
| Aflatoxin B1 aldehyde reductase | Not Found | Combo 1 | Not Found |
| Alcohol dehydrogenase 1 | Combo 1 | Not Found | Not Found |
| Aldehyde dehydrogenase 2 | Not Found | Not Found | Combo 1 |
| Aldehyde dehydrogenase, microsomal | Not Found | Combo 1 | Not Found |
| Alpha 1 - inhibitor III | Not Found | Combo 2 | Not Found |
| Alpha-1 microglobulin/bikunin precursor (Ambp) | Not Found | Not Found | Combo 1 |
| Alpha-2-macroglobulin | Not Found | Combo 1 | Not Found |
| Alpha-2-macroglobulin, sequence 2 | Combo 4 | Not Found | Combo 2 |
| Alpha-2-microglobulin | Not Found | Not Found | Combo 3 |
| Alpha-prothymosin | Not Found | Not Found | Combo 1 |
| Alpha-tubulin | Not Found | Not Found | Combo 3 |
| Annexin V | Not Found | Not Found | Combo 2 |
| Apolipoprotein AII | Not Found | Not Found | Combo 1 |
| Apolipoprotein C1 | Not Found | Not Found | Combo 1 |
| Apolipoprotein CIII | Not Found | Combo 1 | Combo 1 |
| Argininosuccinate lyase | Combo 5 | Combo 1 | Not Found |
| Arginosuccinate synthetase 1 | Not Found | Not Found | Combo 1 |
| ATPase inhibitor (rat mitochondrial IF1 protein) | Not Found | Combo 1 | Combo 1 |
| Bax (alpha) | Not Found | Combo 2 | Not Found |
| Beta-actin | Not Found | Combo 2 | Combo 2 |
| Beta-actin, sequence 2 | Not Found | Not Found | Combo 2 |
| Betaine homocysteine methyltransferase (BHMT) | Not Found | Not Found | Combo 1 |
| Beta-tubulin, class I | Not Found | Combo 1 | Combo 2 |
| Biliverdin reductase | Not Found | Not Found | Combo 3 |
| C4b-binding protein | Combo 2 | Not Found | Not Found |
| Calpactin I heavy chain | Not Found | Combo 1 | Combo 5 |
| Calpain 2 | Not Found | Not Found | Combo 2 |
| Carbamyl phosphate synthetase I | Not Found | Combo 1 | Not Found |
| Carbonic anhydrase III | Combo 2 | Combo 2 | Not Found |
| Carbonyl reductase | Not Found | Combo 1 | Not Found |
| Carnitine palmitoyl-CoA transferase | Combo 1 | Not Found | Not Found |
| Caspase 6 | Combo 1 | Not Found | Not Found |
| Cathepsin B | Not Found | Not Found | Combo 1 |
| Cathepsin L, sequence 2 | Combo 5 | Combo 4 | Not Found |
| Cathepsin S | Not Found | Not Found | Combo 1 |
| Cholesterol 7-alpha-hydroxylase (P450 VII) | Combo 2 | Not Found | Not Found |
| Cholesterol esterase | Not Found | Not Found | Combo 1 |
| Choline kinase | Combo 1 | Not Found | Not Found |
| c-H-ras | Not Found | Combo 1 | Not Found |
| c-jun | Combo 4 | Combo 1 | Not Found |
| c-myc | Combo 5 | Combo 2 | Not Found |
| Cofilin | Not Found | Combo 1 | Combo 3 |
| Collagen type II | Not Found | Not Found | Combo 4 |
| Connexin-32 | Not Found | Not Found | Combo 1 |
| Contrapsin-like protease inhibitor (CPi-21) | Not Found | Not Found | Combo 1 |
| C-reactive protein | Not Found | Not Found | Combo 1 |
| Cyclin D1 | Not Found | Not Found | Combo 2 |
| Cyclin D3 | Combo 1 | Not Found | Not Found |
| Cyclin dependent kinase 4 | Combo 3 | Not Found | Not Found |
| Cyclin G | Not Found | Combo 1 | Combo 1 |
| Cystatin C | Not Found | Not Found | Combo 2 |
| Cytochrome P450 1A1 | Combo 2 | Not Found | Not Found |
| Cytochrome P450 2C11 | Not Found | Not Found | Combo 2 |
| Cytochrome P450 2C23 | Not Found | Not Found | Combo 1 |
| Cytochrome P450 2D18 | Not Found | Not Found | Combo 1 |
| Cytochrome P450 2E1 | Combo 1 | Not Found | Not Found |
| DNA polymerase beta | Not Found | Combo 1 | Not Found |
| DNA topoisomerase I | Combo 2 | Not Found | Not Found |
| Dynamin-1 (D100) | Not Found | Combo 3 | Combo 1 |
| Elongation factor-1 alpha | Combo 1 | Combo 1 | Not Found |
| Endogenous retroviral sequence, 5′ and 3′ LTR | Not Found | Combo 1 | Not Found |
| Enolase alpha | Not Found | Combo 1 | Not Found |
| Epidermal growth factor | Not Found | Combo 2 | Not Found |
| Equilbrative nitrobenzylthioinosine-sensitive nucleoside transporter | Not Found | Combo 2 | Combo 1 |
| Extracellular-signal-regulated kinase 1 | Not Found | Combo 1 | Not Found |
| Fas antigen | Not Found | Combo 1 | Not Found |
| Fatty acid synthase | Not Found | Not Found | Combo 1 |
| Ferritin H-chain | Combo 2 | Not Found | Not Found |
| Focal adhesion kinase (pp125FAK) | Combo 3 | Not Found | Not Found |
| Gadd153 | Combo 5 | Combo 1 | Not Found |
| Gadd45 | Combo 5 | Combo 4 | Not Found |
| Gamma-actin, cytoplasmic | Not Found | Combo 5 | Combo 4 |
| Gap junction membrane channel protein beta 1 (Gjb1) | Not Found | Not Found | Combo 1 |
| Glucokinase | Combo 3 | Not Found | Not Found |
| Glucose-regulated protein 78 | Not Found | Combo 1 | Not Found |
| Glutathione S-transferase theta-1 | Not Found | Not Found | Combo 2 |
| Glycine methyltransferase | Not Found | Not Found | Combo 4 |
| Heme oxygenase | Combo 5 | Combo 2 | Combo 3 |
| Hepatic lipase | Not Found | Combo 2 | Not Found |
| High affinity IgE receptor gamma chain (FcERIgamma) | Not Found | Not Found | Combo 4 |
| H-rev107 | Combo 1 | Not Found | Not Found |
| Hypoxanthine-guanine phosphoribosyltransferase | Not Found | Not Found | Combo 1 |
| ID-1 | Combo 2 | Combo 2 | Not Found |
| IgE binding protein | Not Found | Combo 1 | Combo 4 |
| IkB-a | Not Found | Combo 1 | Not Found |
| Insulin-like growth factor binding protein 1 | Combo 5 | Combo 3 | Not Found |
| Insulin-like growth factor binding protein 3 | Not Found | Combo 2 | Not Found |
| Insulin-like growth factor binding protein 5 | Combo 1 | Not Found | Not Found |
| Insulin-like growth factor I | Not Found | Combo 1 | Combo 1 |
| Insulin-like growth factor I, exon 6 | Not Found | Not Found | Combo 2 |
| Integrin beta1 | Combo 3 | Combo 2 | Not Found |
| Interferon related developmental regulator IFRD1 (PC4) | Combo 3 | Not Found | Not Found |
| Interleukin-18 | Not Found | Not Found | Combo 1 |
| Iron-responsive element-binding protein | Combo 2 | Not Found | Not Found |
| JNK1 stress activated protein kinase | Not Found | Not Found | Combo 1 |
| Lecithin:cholesterol acyltransferase | Not Found | Not Found | Combo 1 |
| L-gulono-gamma-lactone oxidase | Not Found | Combo 3 | Combo 1 |
| Liver fatty acid binding protein | Not Found | Combo 1 | Not Found |
| Macrophage inflammatory protein-1 alpha | Combo 2 | Combo 1 | Not Found |
| Macrophage inflammatory protein-2 alpha | Not Found | Combo 1 | Not Found |
| MAP kinase kinase | Not Found | Combo 1 | Not Found |
| Matrin F/G | Not Found | Combo 5 | Combo 1 |
| Matrix metalloproteinase-1 | Combo 1 | Combo 1 | Not Found |
| Melanoma-associated antigen ME491 | Combo 1 | Combo 1 | Combo 3 |
| MHC class I antigen RT1.A1(f) alpha-chain | Combo 1 | Not Found | Not Found |
| Monocyte chemotactic protein receptor (CCR2) | Not Found | Combo 1 | Not Found |
| Multidrug resistant protein-1 | Not Found | Combo 1 | Combo 2 |
| Multidrug resistant protein-2 | Not Found | Combo 1 | Combo 3 |
| NADH-cytochrome b5 reductase | Not Found | Not Found | Combo 1 |
| NADPH quinone oxidoreductase-1 (DT-diaphorase) | Not Found | Combo 1 | Not Found |
| Neuropeptide Y | Combo 1 | Not Found | Not Found |
| NGF-inducible anti-proliferative putative secreted protein (PC3) | Combo 3 | Not Found | Not Found |
| N-hydroxy-2-acetylaminofluorene sulfotransferase (ST1C1) | Not Found | Combo 2 | Combo 1 |
| NIPK | Combo 5 | Not Found | Not Found |
| Nucleoside diphosphate kinase beta isoform | Not Found | Combo 1 | Combo 2 |
| Nucleosome assembly protein | Combo 2 | Not Found | Not Found |
| Organic anion transporter 3 | Not Found | Combo 2 | Not Found |
| Organic cation transporter 3 | Not Found | Not Found | Combo 2 |
| Ornithine decarboxylase | Not Found | Combo 3 | Not Found |
| Osteoactivin | Not Found | Not Found | Combo 5 |
| p53 | Not Found | Combo 1 | Combo 1 |
| P55CDC | Not Found | Not Found | Combo 1 |
| PAR interacting protein | Not Found | Combo 3 | Not Found |
| Paraoxonase 1 | Not Found | Combo 2 | Not Found |
| Peroxisomal multifunctional enzyme type II | Combo 3 | Not Found | Not Found |
| Phase-1 RCT 252 | Not Found | Combo 1 | Not Found |
| Phase-1 RCT-102 | Not Found | Combo 2 | Combo 1 |
| Phase-1 RCT-107 | Not Found | Not Found | Combo 2 |
| Phase-1 RCT-109 | Combo 1 | Combo 1 | Combo 1 |
| Phase-1 RCT-110 | Combo 2 | Not Found | Not Found |
| Phase-1 RCT-116 | Combo 1 | Not Found | Not Found |
| Phase-1 RCT-117 | Not Found | Combo 2 | Not Found |
| Phase-1 RCT-12 | Not Found | Combo 1 | Combo 2 |
| Phase-1 RCT-121 | Not Found | Not Found | Combo 3 |
| Phase-1 RCT-123 | Combo 2 | Combo 2 | Not Found |
| Phase-1 RCT-127 | Combo 4 | Combo 1 | Not Found |
| Phase-1 RCT-128 | Not Found | Combo 3 | Not Found |
| Phase-1 RCT-137 | Not Found | Combo 1 | Not Found |
| Phase-1 RGT-138 | Not Found | Not Found | Combo 3 |
| Phase-1 RCT-144 | Combo 1 | Combo 4 | Combo 2 |
| Phase-1 RCT-145 | Not Found | Combo 4 | Combo 1 |
| Phase-1 RCT-146 | Not Found | Not Found | Combo 3 |
| Phase-1 RCT-149 | Not Found | Not Found | Combo 1 |
| Phase-1 RCT-15 | Combo 2 | Combo 1 | Not Found |
| Phase-1 RCT-152 | Not Found | Combo 2 | Not Found |
| Phase-1 RCT-154 | Not Found | Combo 1 | Combo 2 |
| Phase-1 RCT-162 | Not Found | Combo 1 | Not Found |
| Phase-1 RCT-164 | Not Found | Not Found | Combo 1 |
| Phase-1 RCT-168 | Not Found | Combo 1 | Not Found |
| Phase-1 RCT-169 | Combo 2 | Not Found | Not Found |
| Phase-1 RCT-173 | Not Found | Not Found | Combo 1 |
| Phase-1 RCT-177 | Combo 2 | Not Found | Not Found |
| Phase-1 RCT-179 | Combo 2 | Combo 2 | Combo 4 |
| Phase-1 RCT-18 | Combo 3 | Not Found | Not Found |
| Phase-1 RCT-180 | Not Found | Combo 3 | Not Found |
| Phase-1 RCT-181 | Not Found | Combo 1 | Not Found |
| Phase-1 RCT-182 | Combo 2 | Combo 3 | Not Found |
| Phase-1 RCT-185 | Not Found | Combo 1 | Combo 1 |
| Phase-1 RCT-187 | Not Found | Not Found | Combo 1 |
| Phase-1 RCT-189 | Not Found | Combo 2 | Not Found |
| Phase-1 RCT-191 | Combo 1 | Combo 2 | Not Found |
| Phase-1 RCT-192 | Not Found | Combo 1 | Combo 4 |
| Phase-1 RCT-197 | Combo 2 | Not Found | Not Found |
| Phase-1 RCT-20 | Combo 1 | Not Found | Not Found |
| Phase-1 RCT-204 | Combo 1 | Not Found | Not Found |
| Phase-1 RCT-205 | Not Found | Combo 1 | Not Found |
| Phase-1 RCT-207 | Combo 5 | Combo 3 | Combo 2 |
| Phase-1 RCT-211 | Not Found | Not Found | Combo 2 |
| Phase-1 RCT-213 | Not Found | Combo 3 | Not Found |
| Phase-1 RCT-214 | Combo 2 | Combo 1 | Not Found |
| Phase-1 RCT-215 | Not Found | Not Found | Combo 2 |
| Phase-1 RCT-219 | Not Found | Not Found | Combo 1 |
| Phase-1 RCT-221 | Combo 1 | Not Found | Not Found |
| Phase-1 RCT-225 | Combo 1 | Combo 1 | Not Found |
| Phase-1 RCT-227 | Combo 1 | Not Found | Combo 1 |
| Phase-1 RCT-230 | Not Found | Not Found | Combo 1 |
| Phase-1 RCT-239 | Not Found | Combo 1 | Not Found |
| Phase-1 RCT-24 | Not Found | Not Found | Combo 2 |
| Phase-1 RCT-241 | Not Found | Combo 2 | Not Found |
| Phase-1 RCT-242 | Combo 4 | Combo 1 | Not Found |
| Phase-1 RCT-248 | Combo 1 | Not Found | Not Found |
| Phase-1 RCT-256 | Not Found | Combo 3 | Combo 1 |
| Phase-1 RCT-258 | Not Found | Combo 3 | Not Found |
| Phase-1 RCT-264 | Not Found | Combo 3 | Not Found |
| Phase-1 RCT-270 | Combo 1 | Combo 2 | Not Found |
| Phase-1 RCT-271 | Not Found | Combo 3 | Not Found |
| Phase-1 RCT-277 | Combo 1 | Not Found | Not Found |
| Phase-1 RCT-278 | Not Found | Not Found | Combo 1 |
| Phase-1 RCT-285 | Not Found | Not Found | Combo 1 |
| Phase-1 RCT-287 | Combo 1 | Not Found | Not Found |
| Phase-1 RCT-288 | Not Found | Combo 3 | Not Found |
| Phase-1 RCT-289 | Combo 1 | Not Found | Not Found |
| Phase-1 RCT-290 | Not Found | Not Found | Combo 1 |
| Phase-1 RCT-291 | Not Found | Combo 2 | Not Found |
| Phase-1 RCT-292 | Not Found | Not Found | Combo 1 |
| Phase-1 RCT-293 | Not Found | Not Found | Combo 1 |
| Phase-1 RCT-295 | Not Found | Combo 2 | Combo 3 |
| Phase-1 RCT-296 | Not Found | Combo 2 | Not Found |
| Phase-1 RCT-33 | Not Found | Combo 3 | Combo 1 |
| Phase-1 RCT-34 | Combo 1 | Not Found | Not Found |
| Phase-1 RCT-36 | Not Found | Combo 3 | Combo 1 |
| Phase-1 RCT-37 | Not Found | Combo 1 | Combo 1 |
| Phase-1 RCT-38 | Not Found | Combo 3 | Combo 1 |
| Phase-1 RCT-39 | Not Found | Combo 3 | Combo 3 |
| Phase-1 RCT-40 | Combo 1 | Combo 2 | Not Found |
| Phase-1 RCT-48 | Not Found | Combo 2 | Combo 1 |
| Phase-1 RCT-49 | Combo 3 | Combo 2 | Not Found |
| Phase-1 RCT-50 | Combo 5 | Combo 4 | Not Found |
| Phase-1 RCT-55 | Not Found | Combo 1 | Not Found |
| Phase-1 RCT-58 | Not Found | Not Found | Combo 1 |
| Phase-1 RCT-59 | Combo 3 | Not Found | Not Found |
| Phase-1 RCT-61 | Not Found | Not Found | Combo 1 |
| Phase-1 RCT-65 | Combo 2 | Combo 1 | Not Found |
| Phase-1 RCT-66 | Combo 1 | Not Found | Not Found |
| Phase-1 RCT-68 | Not Found | Combo 3 | Combo 3 |
| Phase-1 RCT-70 | Combo 1 | Not Found | Not Found |
| Phase-1 RCT-71 | Combo 2 | Not Found | Not Found |
| Phase-1 RCT-72 | Combo 3 | Combo 1 | Not Found |
| Phase-1 RCT-73 | Combo 1 | Not Found | Not Found |
| Phase-1 RCT-75 | Combo 3 | Not Found | Not Found |
| Phase-1 RCT-78 | Not Found | Combo 5 | Combo 2 |
| Phase-1 RCT-81 | Not Found | Not Found | Combo 2 |
| Phase-1 RCT-82 | Combo 4 | Not Found | Not Found |
| Phase-1 RCT-83 | Not Found | Combo 2 | Not Found |
| Phase-1 RCT-87 | Combo 1 | Not Found | Not Found |
| Phase-1 RCT-88 | Not Found | Combo 1 | Not Found |
| Phase-1 RCT-89 | Not Found | Combo 3 | Not Found |
| Phase-1 RCT-92 | Not Found | Combo 4 | Not Found |
| Preproalbumin | Combo 1 | Not Found | Not Found |
| Proliferating cell nuclear antigen gene | Combo 3 | Combo 1 | Combo 1 |
| Protein kinase C alpha | Combo 1 | Not Found | Not Found |
| Protein tyrosine phosphatase, receptor type, D | Not Found | Not Found | Combo 1 |
| PTEN/MMAC1 | Not Found | Not Found | Combo 1 |
| Pyruvate kinase, muscle | Combo 4 | Combo 1 | Combo 1 |
| Ref-1 | Not Found | Combo 1 | Not Found |
| Retinol dehydrogenase type III | Not Found | Not Found | Combo 1 |
| Ribosomal protein L13A | Combo 1 | Combo 1 | Not Found |
| Ribosomal protein S17 | Not Found | Combo 2 | Not Found |
| Ribosomal protein S8 | Not Found | Combo 1 | Combo 1 |
| Ribosomal protein S9 | Not Found | Combo 1 | Combo 3 |
| Sarcoplasmic reticulum calcium ATPase | Combo 3 | Not Found | Not Found |
| Selenoprotein P | Combo 1 | Not Found | Not Found |
| Senescence marker protein-30 | Combo 3 | Combo 2 | Not Found |
| Sodium/bile acid cotransporter | Not Found | Combo 1 | Not Found |
| Stathmin | Not Found | Not Found | Combo 4 |
| Stearyl-CoA desaturase, liver | Not Found | Not Found | Combo 1 |
| Superoxide dismutase Mn | Not Found | Combo 1 | Not Found |
| T-cell cyclophilin | Not Found | Combo 1 | Not Found |
| Thymidylate synthase | Not Found | Not Found | Combo 1 |
| Thymosin beta-10 | Not Found | Combo 1 | Combo 4 |
| Transthyretin | Not Found | Combo 1 | Not Found |
| Tryptophan hydroxylase | Combo 1 | Not Found | Not Found |
| Ubiquitin conjugating enzyme (RAD 6 homologue) | Not Found | Combo 1 | Combo 1 |
| Uncoupling protein 2 | Not Found | Combo 1 | Combo 4 |
| Zinc finger protein | Combo 4 | Combo 4 | Combo 1 |

TABLE 39
9 Liver Predictive Genes that are Predictive
Across all Three Time Points

| Gene | 6 h | 24 h | 72 h |
|---|---|---|---|
| 14-3-3 zeta | Combo 2 | Combo 3 | Combo 2 |
| Heme oxygenase | Combo 5 | Combo 2 | Combo 3 |
| Melanoma-associated | Combo 1 | Combo 1 | Combo 3 |
| antigen ME491 | | | |
| Phase-1 RCT-109 | Combo 1 | Combo 1 | Combo 1 |
| Phase-1 RCT-144 | Combo 1 | Combo 4 | Combo 2 |
| Phase-1 RCT-179 | Combo 2 | Combo 2 | Combo 4 |
| Phase-1 RCT-207 | Combo 5 | Combo 3 | Combo 2 |
| Pyruvate kinase, muscle | Combo 4 | Combo 1 | Combo 1 |
| Zinc finger protein | Combo 4 | Combo 4 | Combo 1 |

TABLE 40
Liver Predictive Genes that are Most Predictive
Across all Three Time Points

| | | | |
|---|---|---|---|
| Heme oxygenase | Combo 5 | Combo 2 | Combo 3 |
| Phase-1 RCT-207 | Combo 5 | Combo 3 | Combo 2 |
| Zinc finger protein | Combo 4 | Combo 4 | Combo 1 |

TABLE 32

| Genes predictive for liver necrosis, sequences, and accession numbers | | |
|---|---|---|
| | Accession | |
| Phase-1 Gene Name | Number | Sequence |
| 14-3-3 zeta | D17615 | TGGNGAATTGGGCCCTCTAGATGCATGCTCGAGCGGCCGCCAGTGTGATGGATATCTGCAGAATTCGCCCTTCGCGG |
| | | GATCCAAAAAGCAGCAGATGGCTCGAGAATACAGAGAGAAGATCGAGACGGAGCTGAGGGACATCTGCAACGACGTA |
| | | CTGTCTCTTTTGGAAAAGTTCTTGATCCCCAATGCTTCGCAGCCAGAAAGCAAAGTCTTCTATTTGAAAATGAAGGGTGA |
| | | CTACTACCGCTACTTGGCTGAGGTTGCTGCTGGTGATGACAAGAAAGGAATTGTGGACCAGTCACAGCAAGCATACCA |
| | | AGAAGCATTTGAAATCAGCAAAAAGGAGATGCAGCCGACACACCCCATCAGACTGGGTCTGGCCCTCAACTTCTCTGT |
| | | GTTCTACTATGAGATCCTGAACTCCCCAGAGAAAGCCTGCTCTCTTGCAAAAACAGCTTTTGATGAAGCCATTGCTGAA |
| | | CTTGATACATTAAGTGAAGAGTCGTACAAAGACAGCACGCTAATAATGCAGTTACTGAGAGACAACTTGACATTGTGGA |
| | | CATCGGATACCCAAGGAGACGAAGCAGAAAAGCTTGGCCAAGGGCGAATTCCAGCACACTGGCGGGCCGNACTAGTG |
| | | GATNCGAGCTCNGTACCCAGCTTTGATGCATA |
| 25-DX | U63315 | TGCGATTGGGCCCTCTAGATGCATGCTCGAGCGGCCGCCAGTGTGATGGATATCTGCAGAATTCGCCCTTACACACAC |
| | | AGTGGCCCCAAATGGTCAATGTACTAAGAATGAAGAGAGAAGGTCTTAGCCATGCTATAGTCTGAAAACAAGCCCATTT |
| | | TACCCAACAGACTTAACTGCATGATTTTTGTTTTATCTACCTCTAAAGCAAAACTGCAGTGTTCCAAAGTCTGTGGTATT |
| | | GATTCAAAACAGAAGTCCAGTAACAAAATGAAAACTCAATAATGGGTTTAGTTGGGGCAAACACATTGCCTGTGTTCAT |
| | | GGACTGATTTATCATCCCTGTCCCCACGTGGACACTCCCACACACAGTAACTCTCACACACCTGGTAATTGGCAGTTGG |
| | | AACTACAACAGAATCTGAAAATTCAAGGTAGAACTTTGCAAAAGAAAAATCTGTTGCATGTAGCAGGGCAATGGTTATG |
| | | TGGTTATTGGCCAAATGTAAAATTTGAGAGCAATATACAGGACAGGAACAGGTGTGCGCTAAGGGCGAATTCCAGCAC |
| | | ACTGGCGGCCGTTACTAGTGGATCCGAGCTCGGTACCAAGCTTGATGCATAGCTTGAGTATTCTATAGTGGCACCTAA |
| | | ATAGCTTGGCGTAATCATGGNCATAGCTGTTTCCTGTGTGAAATTGGTATTCCGCTCACAATTCCACACAACATCCGAG |
| | | CCGGAAAGCATTAAGTGT |
| 3-beta-hydroxysteroid dehydrogenase (HSD3B1) | AA923963 | NGCCAAGCTAAAATTAACCCTCACTAAAGGGAATAAGCTNGCGNCCCGCAANGNTATTTNTTTNATANTTTTTTNNNTTA |
| | | AAGCCATTACAATATTTATTGCTTTATAAATCAATGAGATATAACCAAAGCAGGATGTGATTTAGGACTTGAAGAGGAAC |
| | | AGAAAAGTAATACTCAGCTCTAAGTGACAGGAAATTGTCATTGCTGAAGCCTTTGGTCACAGCAGCTGAGGCACAACTA |
| | | CCTGTGTGTCTCTGGACAGGGGATTAGGGAAGAAAGCTTGTGGACTAGCAAGGCTTCCAGTGAAGTCATAAAGACATA |
| | | GAGTTAGAGTCTGTGTCAAAAGAGGGCATCAGGACCTGGATTGTGCCTCTGTCCTAGCTGGAGGACCTGGTAACACCC |
| | | AGAACCACATCCTTGCCCCCTTTCTGTCACTGAGACTTTGTGTCCAGTGTCTCCCTGTGCTGCTCCACTAGTGTCCCGA |
| | | TCCACTCCGAGGTTTTCTGCTTGGCTTCCTCCCAGCTGACAAGTGGCACATAGCCCAGATCTCTCTGAGCTTTCTTGTA |
| | | GGAGAAAGTGAACTTGCTATTTGACAGTGTGACCAAGTGGCAGTTAAAGGGTGGCCTATAGTTGTAAAATCCTCGTGC |
| | | CGAATTCTTGGCCTCGAGGGCCAAATTCCCTATAGTGAGTAGTATTAAATTCGTAATCATGTCATAGAG |
| 3-hydroxyisobutyrate dehydrogenase | J04628 | CCNNNNCTATGACATGATTACGAATTTACTACGACTCACTATAGGGAATTTGGCCCTCGAGGCCAAGAATTCGGCACGA |
| | | GGAGCGACACTTACAACCCTGTACCTGGGGTTATGGATGGAGTTCCCTCATCTAATAACTACCAAGGTGGATTTGGGA |
| | | CAACACTCATGGCCAAGGATCTGGGATTAGCTCAAGACTCTGCCACCAGCACCAAGACCCCAATTCTCCTCGGAAGCG |
| | | TGGCCCATCAGATATACAGGATGATGTGTTCAAAGGGCTACTCAAAGAAAGACTTCTCCTCTGTGTTCCAGTATCTGCG |
| | | GGAGGAGGAAACCTTCTGACTGCCCTGTGGTCATGGACACTGTTAGGAACCAAACTCTGTCCTGAGCTTCCTTCTAGC |
| | | TCATTCTAGAAGTCATGGGTTTAATCAAAGGTCATCTGTCTGCTTTTGATTGTCTACATTATAGCAATCCCTGGGGATTTT |
| | | TATCCATTTTAAATGCTGCTGCTTTCATCTGTTTAGCAAACACACCCAATGGTTTCCACTGACTAGCCCAGTTGACCTTTT |
| | | TTAAGTTTGAACCTTGAGCACCTTAAACAAAATTGAGCACTCTGATCAGGATATTTTATTTTACTCTGCTTTACNAATAAA |
| | | ACCAAATGGTTTGTTTAACAAGATGGAACCATGGTAAAAAAGGAAAAAAATCAGTTN |
| 60S ribosomal protein L6 | X87107 | ANCGGGCCCTCTAGATGCATGCTCGAGCGGCCGCCAGTGTGATGGATATCTGCAGAATTCGCCCTTCGCGGGATCCG |
| | | GTGAAGCTTCGAAAAATGCCTAGGTATTACCCTACTGAAGACGTGCCTCGGAAGCTGCTGAGCCACGGCAAGAAGCCC |
| | | TTCAGCCAGCACGTGAGGAGGCTGCGCTCCAGCATCACTCCCGGGACTGTCCTGATCATCCTCACTGGGCGCCGCAG |
| | | GGGCAAGAGAGTGGTTTTCCTCAAGCAGCTGGGCAGTGGCTTGCTACTTGTGACTGGACCTCTTGCCCCCAACAGAGT |
| | | TCCTCTGCGTAGGACACACCAGAAGTTTGTCATCGCCACCTCTACAAAAGTTGATATCAGCAAGGTTAAAATTCCCAAA |
| | | CACCTGACTGATGCTTACTTCAAGAAGAAGCCACTTCGCAAGCCCAGGCATCAGGAGGGTGAGATCTTCGACACAGAG |
| | | AAGGAGAAATACGAAATTACAGAGCAGCGAAAGGCTGATCAGAAAGCTGTGGACTCGCAGATTTTGCCAAAGATCAAA |
| | | AGCTGTCCCCCAGCTTCAGGGCTACCTGCTTGCANGGCCAANGGCGAATTCAGCACACTTGGCGGGCGGTACTAGTG |
| | | GGATNCCN |
| 60S ribosomal protein L6 (alternate clone 1) | X87107 | CTATGACATGATTACGAATTTAATACGACTCACTATAGGGAATTTGGCCCTCGAGGCCAAGAATTCGGCACGAGGAGCT |
| | | GAGGAAGAGTAAGCCCCATTGCAGCCGGAACCCTGTCCTGGTGAGAGGAATCGGCAGGTATTCACGATCTGCTATGT |
| | | ATTCCAGAAAGGCCTTGTACAAAAGGAAATACTCTGCTGCCAAGACAAAGGTTGAGAAGAAGAAGAAGAAAGAAAAGG |
| | | TCCTTGCTACCGTCACAAAAACAGTTGGTGGGGACAAGAACGGTGGCACCCGGGTGGTGAAGCTTCGAAAAATGCCTA |
| | | GGTATTACCCTACTGAAGACGTGCCTCGGAAGCTGCTGAGCCACGGCAAGAAGCCCTTCAGCCAGCACGTGAGGAGG |
| | | CTGCGCTCCAGCATCACTCCCGGGACTGTCCTGATCATCCTCACTGGGCGCCACAGGGGCAAGAGAGTGGTTTTCCT |
| | | CAAGCAGCTGGGCAGTGGCTTGCTACTTGTGACTGGACCTCTTGCCCTCAACAGAGTTCCTCTGCGTAGGACACACCA |
| | | GAAGTTTGTCATCGCCACCTCTACAAAAGTTGATATCAGCAAGGGTAAAATTCCCAAACACCTGACTGATGCTTACTTCA |
| | | AGAAGAAGCCACTTCGCAAGCCCAGGCATTAGGANGGTGAGA |
| 8-oxoguanine DNA glycosylase | AF029690 | NAGTGNCTCCTCTAGATGCATGCTCGAGCGGCCGCCAGTGTGATGGATATCTGCAGAATTCGCCCTTTGCAGCAGCTT |
| | | CGAGTGGCCTCTTATGAAGAGGCCCACAAGGCCCTCTGCACCCTGCCTGGAGTGGGCACCAAGGTGGCTGACTGCAT |
| | | CTGCTTAATGGCCCTGGACAAACCCCAGGCTGTGCCTGTAGACATCCATGTGTGGCAGATTGCCCATCGTGACTACGG |
| | | CTGGCAGCCTAAGACATCGCAGACTAAGGGCCCGAGCCCTCTGGCCAACAAAGAACTGGGAAACTTTTTCCGGAATCT |
| | | GTGGGGACCTTATGCTGGCTGGGCCCAAGCAGTGCTGTTCAGTGCTGACCTTCGTCAACAAAACCTGTCTCGGGAGC |
| | | CACCAGCAAAGCGCAAAAAGGGATCTAAGAAGACAGAAGGCTAGGTAGGGCCCAGGGGACAAAAGATGTCACTGATA |
| | | TTTTCCCCTTTGTTCTCCATTTCTCTCCCTTGTCCTCACCCAAGTCAGAAAGGCTTAACATTCCTACCTCCAAGGCAAAC |
| | | ATCCCAAATCAAAGGGCGAATTCCAGCACACTGGCGGCCGGTACTAGTGGATCCGAGCTCGGTACCAAGCTTGATGC |
| | | ATAGCTTGAGTATTCTATAGTGTCACCTAAATAGTTGGCGTAATCATGGCATAGCTGGTTCCTGTGTGAAATTGTAN |
| Acetyl-CoA carboxylase | AA925393 | ATCGATTTCTCACTGACACGAGTGAAGGCTGCTTTGTGAACAAGTGGACAGATAATATGGCAATCTCAGTTCAAGCAAG |
| | | TCTGGGTCAACACATCATTCATCCTCCTCCCTCAGTAACCTTGCATGTGTAATGGGCTGTGGGGCCATCAGACTGCTCA |
| | | TGTGCTGTGGCTCTGACTTCTCCATGGAGGAGCCCCGGTCCTGGGGATGGGGCAGCAGGGAAAAGGGAGTTATACAA |
| | | GCACAACCCACTTCACCTCAGAAGGAACGGCAGGAAAGCAGCCTTCATACCACTGGGTGTGGGACTGGATGTGCCGT |
| | | AGCAAAGTGTAGAGGCTGGAGACAAATCCCACCCTTACCTACAAGGTGAGCTGAGAACCAGGGGCCAGGGAGCTTCC |
| | | TTCTAACTGAGGATGCTCCCCACCTGATCTGGCCCTGGCCCTCCTGTTCGCTTGTAGGAGTTGGGCAACTTGCATCTAT |
| | | AGTCCCTTGGATGTCCGCTTGCAATGTGGCACCATTGAGAAGGTCCTCTGGAATATCTGCTCGAGGTGCTCCTGAATG |
| | | GGAGGTCAGCTCTTTTTGTGGAGTCCAGGTTAAGGCTCAGACTCCTCGTGCCGAATTCTTGGCCTCGAGGGCCAAATT |
| | | CCCTATAGTGAGTCGTATTAAATTCGTAATCATGTCATAGA |
| Activating transcription factor 3 | M63282 | AGCTATGNCCATGATTACGCCAAGCTATTTAGGTGCCACTATAGAATACTCAAGTATGCATCAAGTTGGTACCGAGCTC |
| | | GGATCCACTAGTAACGGCCGCAGTGTGCTGGAATTCGCCCTTTGCGGCTGACAACATCCCTCCTAGGGAAGATGGAG |
| | | TGAGAACATTCATCATTGAAGTTGTCCAATGGCCAGGGTATGCTTTCTAGAAACTATGCTGTTCTGTCCTAGACTGACTG |
| | | TGCATAGGGCATTCGTTTCTGAGCCTGGTGTTGTGCTATTTAGATGTTTGTCTTGCACAACATTGGCGTGATTTTTTTCC |
| | | GGGAGTTTCATCAGACCTGATTTCCGAGAGTTTGGGGGTCTGCCACTGTGGACAATATCCCCCAAAAGTGTTTGGGTG |
| | | GCCATGTAAACTGGCTGATGACCAGCTGTGCTACTCTGTGCTGACCGAGGACTGATGCCTCCTTCCCCTGTACCCACT |
| | | GCTGAGGAAGAACCCGGGCACAGCAGCTGTCCTTGGCTACAAACTGTTACAATGTCACAGAACGAAGGCACAAAGTCC |
| | | CGCTTTCAAAGGGCGTAGGACTCCACACTCAGTGACAGGGCAGGAAGAGCCAAGGGCGAATTCTGCAGATATCCATC |
| | | ACACTGGCGGCCGCTCGAGCATGCATCTAGAGGGCCCAATTCGCAA |
| Adenine nucleotide translocator 1 | D12770 | GCGAATTGGGCCCTCTAGATGCATGCTCGAGCGGCCGCCAGTGTGATGGATATCTGCAGAATTCGCCCTTTCGCTGGT |
| | | AACCTGGCCTCTGGTGGGGCAGCTGGGGCTACCTCCCTCTGCTTCGTCTACCCACTGGACTTTGCTAGGACCAGGCT |
| | | GGCTGCCGACGTGGGCAAGGGATCTTCCCAGCGTGAGTTCAATGGGCTGGGTGACTGTCTCACCAAGATCTTCAAGT |
| | | CTGATGGCCTGAAGGGTCTCTACCAGGGTTTCAGTGTCTCTGTGCAGGGCATCATCATCTACAGAGCTGCCTACTTCG |
| | | GAGTCTATGACACTGCCAAGGGGATGCTGCCAGACCCCAAGAATGTGCACATTATTGTGAGCTGGATGATTGCCCAGA |
| | | GTGTGACAGCCGTGGCGGGGCTAGTGTCCTATCCATTTGACACTGTCCGTCGTAGGATGATGATGCAGTCTGGCCGG |
| | | AAAGGGGCTGATATTATGTACACGGGGACAGTTGACTGCTGGAGGAAGATTGCAAAAGATGAAGGAGCCAAAGCTTTC |
| | | TTCAAAGGTGCTTGGTCCAACGTACTGAGAGGCATGGGGGGTGCTAAGGGCGAATTNCAACACACTNGCGGGCGGTA |
| | | CTAATGGATCCCANCTCNG |
| Aflatoxin B1 aldehyde reductase | X74673 | GAATTGGGCCCTCTAGATGCATGCTCGAGCGGCCGCCAGTGTGATGGATATCTGCAGAATTCGCCCTTACCCTCTGCA |
| | | AGAAAAATGGCTGGATCATGCCAACTGTGTACCAGGGCATGTACAACGCCATCACCAGGCAGGTGGAGACTGAGCTCT |
| | | TCCCCTGCCTCAGACACTTCGGACTAAGGTTCTACGCCTTCAACCCTTTGGCTGGGGGCCTGCTGACTGGCAGATATA |
| | | AATACCAGGATAAGGATGGGAAGAATCCTGAGAGCCGCTTCTTTGGGAATCCATTTTCTCAACTGTACATGGACCGCTA |
| | | CTGGAAGGAGGAACACTTCAATGGCATCGCCTTGGTGGAGAAGGCTCTGAAGACTACCTATGGCCCCACTGCCCCCA |
| | | GTATGATCTCAGCTGCCGTACGGTGGATGTACCATCACTCACAGCTCAAGGGCACCCAAGGGGATGCAGTCATTCTGG |
| | | GCATGTCCAGTCTGGAACAACTGGAGCAGAACTTGGCCTTGGTCGAGGAAGGGCCTCTGGAGCCAGCTGTTGTGGAT |
| | | GCCTTTGACCAAGAAGGGCGAATTCCAGCACACTGGCGGCCGTTACTAGTGGATCCGAGCTCGGTACCAAGCTTGAT |
| | | GCATAGCTTGAGTATTCTATAGTGTCACCTAAATAGCTTGGCGTAATCATGGNCATAAGCTGGTTTCCTGTGGTGAAATT |
| | | G |
| Alcohol dehydrogenase 1 | M15327 | TAGGTGACACTATAGAATACTCAAGCTATGCATCAAGCTTGGACCGAGCTCGGATCCACTAGTACCGCCCGCCAGTGT |
| | | GCTGGAATNCCCCTTTCGCGGGGATCCAGTCGCCAAGGTGACCCCAGGCTCCACCTGTGCCGTGTTTGGCCTGGGAG |
| | | GTGTTGGTCTGTCTGTCGTCATTGGCTGTAAAACAGCAGGAGCAGCCAAGATCATTGCCGTGGACATCAACAAAGACA |
| | | AGTTTGCGAAGGCCAAAGAGTTAGGTGCCACTGACTGTATCAACCCTCAAGACTACACCAAACCCATCCAGGAAGTTC |
| | | TCAGGAGATGACTGATGGAGGGGTGGACTTTTCATTTGAAGTCATTGGCCGTCTTGATACCATGACTTCTGCCCTGTTA |
| | | AGCTGCCATTCAGCATGCGGTGTAAGCGTCATTGTCGGGGTGCCTCCCAGTGCCCAAAGCCTCTCCGTTAACCCCATG |
| | | TCGCTGCTGCTGGGACGCACCTGGAAAGGAGCAATATTCGGCGGGTTTAAGAGTAAAGATGCCGTCCCCAAACTTGTC |
| | | GCTGACTTCATGGCTAAGAAGTTTCCGTTGGAGCCGCTGATTACTCATGAAGCTTGGCCAAGGGCGAATTCTGCAGAT |
| | | ATCCATCACACTGGCGGCCGCTCGAGCATGCATCTAGAGGGCCCAATTCTCCAA |
| Aldehyde dehydrogenase 2 | X14977 | GCGAATTGGCCCTCTAGATGCATGCTCGAGCGGCCGCCAGTGTGATGGATATCTGCAGAATTCGCCCTTACAAGAAGG |
| | | GGCGAAGCTGCTGTGCGGTGGGGGCGCCGCCGCAGACCGTGGTTACTTCATCCAGCCCACCGTGTTCGGAGACGTC |
| | | AAAGATGGCATGACCATCGCCAAGGAGGAGATCTTCGGACCAGTGATGCAGATCCTCAAATTCAAGACCATTGAGGAG |
| | | GCTGTGGGGCGAGCCAATAATTCCAAGTACGGGCTGGCTGCCGCTGTCTTCACAAAGGACCTGGACAAGGCCAATTA |
| | | CCTGTCCCAAGCTCTGCAGGCTGGGACTGTGTGGATCAACTGCTACGATGTGTTTGGGGCCCAGTCCCCATTTGGTGG |
| | | CTATAAGATGTCGGGGAGCGGCAGGGAGCTGGGCGAGTATGGCCTGCAGGCCTACACGGAAGTGAAGACGGTCACC |
| | | GTCAAAGTGCCACAGAAGAACTCGTAAAGTGGCGTGCAGGCTTCCTCAGCCAGCGCCCAAAAACCCAACAAGATCCTG |
| | | AGAAAAGCCACCACCAAGCACACTGCAAGGGCGAATTCCAGCACACTGGCGGCCGTTACTAGTGGATCGGAGCTCGG |
| | | TACCAAGCTTTGATGCATAGCTTGAGTATTCTATATGGCACCTAA |
| Aldehyde dehydrogenase, microsomal | AA956846 | GCCAATGANTCGTTCTTGATACNTCATACAATACANTTCAGACAGCTTCAACGGGTGGAACAGGTTANTCAGCAAACAT |
| | | GGTTGTACATACGCAAGTGACATTCTCATTGAGTGGGCAGTCTTAATTTGGATCCTGGGAAGGCAATGAGACCTCTGCA |
| | | GCTTGGAGCTTCAGGCCTAACCAGGCCCTGGCAGCAGGGCCACACAGATCTCTCTGCCACATCCCCGTGTAGCCATG |
| | | TTAGGGTCTGACAGAGAATGCTGCCCCGCTTGCTCAGGTGCGCTTCTGGCCATCCCATCGCTTCAGTGTGCCCCCTCA |
| | | GCTCACGGTAGTCTCCAACAACCCTGCCCCATGAGTTCTGAAGCCTCATGTCTAATAACCCCCCTTTCACTACAGGAAC |
| | | AGCTCCATGCTAAGATCTGTAGGAATCTCTCTAAGATTTCAAAAGTAAATTTTTTTTGTGTTGTGCTGTGAAAATAAGCAG |
| | | ATTTATCCTGAAGACCTATATAACATTCAAATCGAGGGTCTCGAGGGGATTGTTATGGAGGAATAATAATGAGAGGGTC |
| | | CCATTATGTTGCCTAGGTTGGCCTCAAACTCCCGGGCTCCGGAATCTTCTGCCCTCGGGCTCTGGCCTCGTGCCGAAT |
| | | TCTTGGCCTCGAGGGCCAAATTCCCTATAGTGAGTCGTATTAAATTCGTAATCATGTCATA |
| Alpha 1 - inhibitor III | J03552 | GAAATNGGCCCTCTAGATGCATGCTCGAGCGGCCGCCAGTGTGATGGATATCTGCAGAATTCGCCCTTATCGCGGGAT |
| | | CCACCGTCAGTGTGTCAGGGGAAGGATGTGTGTATGCTCAGACCATGCTGAGATACAACATGCCCTTGGAGAAGCAG |
| | | CAGCCTGCATTTGCTCTAAAGGTGCAGACAGTACCCCTGACTTGTAACAACCCCAAAGGCCAGAACAGCTTCCAGATC |
| | | TCACTAGAAATCAGTTACACGGGGAGCCGTCCAGCCTCCAACATGGTGATTGCTGATGTGAAGATGCTCTCTGGCTTC |
| | | ATCCCATTGAAACCAACAGTGAAGAAGCTTGAAAGATTAGAGCACGTAAGCAGAACAGAAGTGACCACCAACAACGTC |
| | | CTGTTATATTTGGACCAGGTAACCAATCAGACACTGTCCTTCTCCTTCATCATTCAACAAGACATCCCAGTAAAGAACCT |
| | | GCAGCCTGCCATTGTGAAAGTGTATGACTACTATGAGACAGATGAAGTGGCTTTTGCTGAATACAGCAGCCCCTGCAG |
| | | CTCAGACGACCACAAGGGCGAATTCCAGCACACTGGCGGCCGTTACTAGTGGATCCGAGCTCGGTACCAAGCTTTGA |
| | | TGCATAGCTTGAGTATTCTATAGTGTCACCTAAATAGCTTGGCGTAATCATGGC |
| Alpha-1 microglobulin/bikunin precursor | AI043784 | AGCNNCNCCNCCCCTTGCACGCCATGNTTCCTTCTNTCCTGGGAAGGTGGTTTACTCATGCCCTGAGCTTCCNTTCCT |
| (Ambp) | | NTCCAATACCCTTATTGTGACAATGAGATGCTAACACACAGAATGCTAGGAAATGACAGCTTGTTTTATTGGGTCCAGAT |
| | | CATCTAAGCTGGACTGCACAGTGACGGACCGTGGCTACCCTCTGGCTTGCAGACCGGCTCCTTCAACTGCGTGTTAGC |
| | | TCCTCGTACCCATCACCAGGGACTCCACAGTACTCCTTGCACTCCTTCTCAGAGTAGAACTTGTTGCCGTTGCCTTTGC |
| | | AGCCCCCATAGATGAATTGGATGCACTTCCCTTGCGCTGCATCAAATGCCCAGAGTTCTGCAAAGGCTCGGCAGGGGC |
| | | CTTGGACTATGGGGAGATTGCAGGCCGCTATTGTCCGGCACGTCTGCAGGCATTCCTTCTCGGAGGCGAAGTTGTTAC |
| | | CATTGCCTAGGCAGCCGCCATACTGGAAGGTCTCGCAGGCCATGGAGGCGCCATTGTAGTAATACTTCTGCTGCATCC |
| | | CCAGGCAGGGACCTTCTGAGTAGTTGAGCTGGCAGGAGTCTTCTTTCTTGAGGGTCCCAGTTATGAGTGGCTCACCCT |
| | | CGTGCCGAATTCTTGGCCTCGAGGGCCAAATTCCCTATAGTGAGTCGTATTAAATTCGTAATCATGTCATA |
| Alpha-2-macroglobulin | J02635 | GCGAATTGGGCCCTCTAGATGCATGCTCGAGCGGCCGCCAGTGTGATGGATATCTGCAGAATTCGCCCTTGCCGACT |
| | | GTGCCTGGGGATTACACCGTGAAGGTGACAGGAGAAGGCTGTGTCTACCTCCAGACATCCTTGAAATACAGTGTTCTC |
| | | CCGAGAGAGGAGGAGTTCCCCTTCGCTGTGGTGGTGCAGACTCTGCCTGGGACATGTGAGGATCCCAAAGCTCACAC |
| | | CAGCTTCCAGATCTCACTCAACATCAGTTACACTGGAAGCCGTTCTGAATCCAACATGGCAATTGCTGACGTGAAGATG |
| | | GTGTCCGGCTTCATCCCCTTGAAACCAACAGTGAAAATGCTTGAAAGATCTGTGCATGTGAGCCGAACAGAAGTCAGC |
| | | AATAACCATGTCTTGATTTACCTGGATAAGGTGTCAAATCAGACGGTGAACTTGTCCTTCACGGTTCAGCAAGATATTCC |
| | | AATAAGAGACCTGAAGCCAGCCGTAGTGAAAGTCTACGATTACTATGAGAAAGATGAGTTTGCAGTTGCAAAATACAGC |
| | | GCTCCCTGCAAGGGCGAATTCCAGCACACTGGCGGCCGTTACTAGTGGATCCGAGCTCGGTACCAAGCTTGATGCAT |
| | | AGCTTGAGTATTCTATAGTGTCACCTAAATAGCTTGGCGTAATCATGGGCATAGCTGGTTNCTGTGTGAAATTG |
| Alpha-2-macroglobulin, sequence 2 | NM_012488 | TATGACATGATTACGAATTTAATACGACTCACTATAGGGAATTTGGCCCTCGAGGCCAAGAATTCGGCACGAGGTGCAA |
| | | GGCAGCCAGGTCTCCGATCCTTTCCGCAGCATGGGGAAGCACAGGCTCCGGAGCCTGGCCCTGCTGCCACTGCTGCT |
| | | GCGGCTGCTGCTGCTGCTGCTGCCCACCGATGCCTCAGCTCCACAAAAACCAATCTACATGGTGATGGTTCCCTCCCT |
| | | GCTCCACGCAGGAACCCCCGAGAAGGCCTGCTTCCTGTTCAGCCATCTAAACGAGACAGTGGCTGTGAGAGTGTCCTT |
| | | GGAGTCTGTCCATGGGAACCAAAGCCTCTTCACTGACCTTGTAGTTGACAAGGACCTATTCCACTGTACCTCCTTCACC |
| | | GTCCCACAGTCTTCATCTGATGAGCTGATGTTTTTCACTGTCCAAGTAAAAGGAGCAACTCATGAGTTCTGGCCCCTAA |
| | | GTATCGCCGCCGTCTGTTTTATTTAGTGTACAGTACTATAGGTGCGCACTCTGGTCATTTTCCAAGCCATGTTTTATCAT |
| | | ATCTGTTTTCTACTTTCCGTGAGCGAGGTTTGCTGTCCAAGGTGTAAATACTCATGGGAATAAAACTGGCATGGTACTTT |
| | | CCCTTCCTTTCTCATTTTCTTGGCTCTGAGATTTCAAAGGTAACGGCCCATCAACAAGC |
| Alpha-2-microglobulin | AB039825 | TTCTGACATGATTACGAATTTAATACGACTCACTATAGGGAATTTGGCCCTCGAGGCCAAGAATTCGGCACGAGGATCA |
| | | GCAGAGAGATTGTCCCGACAGAGAGGCAATTCTATTCCCTACCAACATGAAGCTGTTGCTGCTGCTGCTGTGTCTGGG |
| | | CCTGACCCTGGTCTGTGGCCATGCAGAAGAAGCTAGTTTCGAGAGAGGGAACCTCGATGTGGACAAGCTCAATGGGG |
| | | ATTGGTTTTCTATTGTCGTGGCCTCTGATAAAAGAGAAAAGATAGAAGAGAACGGCAGCATGAGAGTTTTTGTGCAGCA |
| | | CATCGATGTCTTGGAGAATTCCTTAGGCTTCACGTTCCGTATTAAGGAAAATGGAGTGTGCACAGAATTTTCTTTGGTTG |
| | | CCGACAAAACAGCAAAGGATGGCGAATATTTTGTTGAGTATGACGGAGAAAATACATTTACTATACTGAAGACAGACTA |
| | | TGACAATTATGTCATGTTTCATCTCGTTAATGTCAACAACGGGGAAACATTCCAGCTGATGGAGCTCTACGGCAGAACA |
| | | AAGGATCTGAGTTCAGACATCAAGGAAAAGTTTGCAAAACTATGTGTGGCACATGGAATCACTAGGGACAATATCATTG |
| | | ACCTAACCAAGACTGATCGCTGTCTCCAGGCCCGANGNTGAANAAANGGCTGAGCCTCCAGANTGCCTCTCTGTCCAG |
| | | AAGTCAATCCAAGAAGT |
| Alpha-prothymosin | AA875070 | TACTGGAATGTCAGAATAAGATTGTTTTTTGGTGGTGTTTTTGTTTTTTTACAAGGTTTTTTTTCTCCTTGAGAGTATAATG |
| | | AACATGGTCACACCACAAGTAAAGTCTGAAGTAGGACAGAAAACGCTCTGAAGGCTGGTTTGGTCACCCGTTATCATTA |
| | | AAAATGGCTGACCCCTAACAATATGTACAAAAATATAAAATGTAAATAAAAAATACAAACAAATTTTCCTTTTAAAGTATTT |
| | | TTAAGAAAAAAAGCAGGGCCTGGGAAGTTCTGGTTCTTTTCTCCTCCCATGTTGCCAATTCATGTTGTGGTTTTGGTGG |
| | | GGTGGTGAAGAGCGCATGTCATGTGTGGGTGGCACTGCCCGCAGTGGGCGGGCGGGGGCCTGCTTCTCTACTCGAA |
| | | GGTGACCACGTTTAAATTCTGAGACGGGAAGTGGAGGGTGAATAGGTCACGGTGCGTAAGGTTAGCTTTTCCTTTTGC |
| | | TGTCTAGTCATCCTCATCAGTCTTCTTCTGCTTCTTGGTCTCAACATCATCATCCTCATCATCCTCAGCTACCCGCTTGC |
| | | CCGTAGGAGCCTCAGCTTCCTCATCTTCATCTCCATCTTCTTCCTCACCCTCGTGCCGAATTCTTGGCCTCGAGGGCCA |
| | | AATTCCCTATAGTGAGTCGTATTAAATTGCGTAATCATGNTNGGAGA |
| Alpha-tubulin | J00798 | GGGAATTGGGCCCTCTAGATGCATGCTCGAGCGGCCGCCAGTGTGATGGATATCTGCAGAATTCGCCCTTGCGCGGA |
| | | TCCTGATGTGGTCCCCAAAGATGTCAATGCTGCCATTGCCACCATCAAGACCAAGCGCAGCATCCAGTTTGTGGACTG |
| | | GTGCCCCACTGGCTTCAAGGTTGGCATTAATTACCAGCCTCCCACTGTGGTACCTGGTGGCGACCTGGCCAAGGTCCA |
| | | GAGAGCTGTGTGTATGCTGAGCAACACCACAGCCATTGCTGAGGCTTGGGCTCGCCTGGATCACAAGTTTGATCTGAT |
| | | GTATGCCAAGCGTGCCTTTGTGCACTGGTACGTGGGTGAGGGAAGCTTGGCCAAGGGCGAATTCCAGCACACTGGCG |
| | | GCCGTTACTAGTGGATCCGAGCTCGGTACCAAGCTTGATGCATAGCTTGAGTATTCTATAGTGTCACCTAAATAGCTTG |
| | | GCGTAATCATGGTCATAGCTGTTTCCTGTGTGAAATTGTTATCCGCTCACAATTCCACACAACATACGAGCCGGAAGCA |
| | | TAAAAGTGTAAAGCCTGGGGTGCCTAATGAGTGAGCTAACTCACATTAATTGCGTTGCGCTCACTGCCCGCTTTNCAAT |
| | | CGGGAAACCTGCGTGCCAACTGCATTAATGAATCGC |
| Annexin V | M21730 | GTGAATTGGCCCTCTAGATGCATGCTCGAGCGGCCGCCAGTGTGATGGATATCTGCAGAATTCGCCCTTATCGCGGGA |
| | | TCCGGGGGACGGATGAAGAGAAGTTCATCACCATCCTTGGGACACGCAGTGTGTCTCATTTAAGAAGAGTGTTTGACA |
| | | AGTACATGACAATATCAGGATTTCAGATTGAGGAAACCATTGACCGAGAGACCTCAGGGAACTTGGAGAACTTACTCCT |
| | | GGCTGTCGTGAAGTCTATTCGGAGCATACCTGCCTACCTTGCAGAGACCCTCTACTATGCTATGAAGGGTGCTGGGAC |
| | | GGACGATCACACCCTCATCAGAGTCATAGTGTCGAGGAGTGAGATTGATCTGTTTAACATCAGGAAGGAGTTTAGGAA |
| | | GAACTTCGCCACGTCCCTGTACTCTATGATCAAGGGCGACACATCTGGAGACTATAAGAAGGCCCTGCTGCTCCTCTG |
| | | TGGAGGCGAGGATGACTGAGGAGCTGCCTGGAGTGCCCTGGGCCCGCCTGCTGCCCACCATCAGCTTCCTTCAGCAC |
| | | CACGCCTACTTACGTTCAATGCCTGCCTGAAGGGCGAATTCCAGCACACTGGCGGCCGTTACTAGTGGATCCGAGCTC |
| | | GGTACCAAGCTTGATGCATAGCTTGAGTATTCTATAGTGNCACCTAAATAGCTTGGCGTAAT |
| Apolipoprotein AII | M28615 | GCGAATTGGGCCCTCTAGATGCATGCTCGAGCGGCCGCCAGTGTGATGGATATCTGCAGAATTCGCCCTTGCGCGGA |
| | | TCCTAGCCTGGAAGGAGCTTTGGTTCGGAGACAGGCAGCGGAGACGGATGTGCAGACCCTGTTCAGCCAGTATCTTC |
| | | AGAGTTTAACTGACTATGGCAAGGATTTGATGGAGAAGGCCCAGCCCTCAGAGATTCAGAACCAAGCCAAGGCTTACT |
| | | TTCAGAATGCACAGGAGAGACTGACACCCTTTGTCCAGAGAACTGGGACGAATCTGATGGACTTCTTAAGCCGTTTAAT |
| | | GAGCCCCGAGGAGAAACCAGCTCCTGCAAGCTTGGCCAAGGGCGAATTCCAGCACACTGGCGGCCGTTACTAGTGGA |
| | | TCCGAGCTCGGTACCAAGCTTGATGCATAGCTTGAGTATTCTATAGTGTCACCTAAATAGCTTGGCGTAATCATGGTCA |
| | | TAGCTGTTTCCTGTGTGAAATTGTTATCCGCTCACAATTCCACACAACATACGAGCCGGAAGCATAAAGTGTAAAGCCT |
| | | GGGGTGCCTAATGAGTGAGCTAACTCACATTAATTGCGTTGCGCTCACTGCCCGCTTTCCAGTCGGGAAACCTGTCGT |
| | | GCCAGCTGCATTAATGAATCGGNCAACGCGCGGGGAGANGCGGNTTGCGTATTGGGCG |
| Apolipoprotein C1 | AA955662 | NCCCNATTCAGGTTGCGCAANTTTTGGGAANGGNCAATCGGTGCGGCCTTNTTCGCTATNACNCCAGCTGGGGAAAG |
| | | GGGNATGTGCTGCAAGGGATTAAGTTGGGGAAACNCCAGGNTTTTCCCAGTCACGACGTTGTAAAACGACGGCCAGT |
| | | GCCAAGTAAAATTAACCCTCATAAAGGGAATAAGCTTGCGGCCGCAATGTTTTNNNNNNNNNNNNNNNNNNNTTCTCTA |
| | | CAAGTTTTTATTGGTATTTGAAGAGGGACCTGGCCCGTCACGTGGCCCTCTTTGCTGCTGCGAGGTGGCTCGCCAGGC |
| | | ACTCAGGCAAATGTGGTTTTTAATTTCTCCTTCATTTTGTTGAGCGTCTCTGAAAACCAGTTCCGAGTCTTGATCATAATT |
| | | TCCTTCTGTTTGATATGCTCAATGGCTGCCCGGGCCTTATCTTCCAAAGTGTTCCCAAACTCTTTTAGTTTATCCGGTAA |
| | | GCTCTCCATTGCGCTGGAAAAGTCAGGGGCCGCCTGGGCAGGGGCTGGGCCTTCCAAAGCCATGGCTACGACCACG |
| | | ATCAGGACAGGAAGTGCGATGAAGAGCCTCATCCTGGCTGGAGGGGCACCCTGGATCTAAGGTTCTCTCCCTCGTGC |
| | | CGAATTCTTGGCCTCGAGGGCCAAATTCCCTATAGTGAGTCGTATTAAATTCGTAATCATGTCATA |
| Apolipoprotein CIII | J02596 | GCAAACCGCTTCTCCCGGGGCGTTGGCCGATTCATTAATGCAGTGGCACGACAGGTTTCCCGACTGAAAAGCGGGCA |
| | | GTGAGCGCAACGCAATTAATGTGAGTTAGCTCACTCATTAGGCACCCCAGGCTTTACACTTTTATGCTTCCGGCTCGTA |
| | | TGTTGTGTGGAATTGTGAGCGGATACCAATTTCACACAGGAAACAGCTATGACCATGATTACGCCAAGCTATTTAGGTG |
| | | ACACTATAGAATACTCAAGCTATGCATCAAGCTTGGTACCGAGCTCGGATCCACTAGTAACGGCCGCCAGTGTGCTGG |
| | | AATTCGCCCTTGCGCGGATCCTCACGGCTCAAGAGTTGGTGTTGTTAGTTGGTCCTCAGGGCCAGACTCCCAGAGGCC |
| | | AGTGAACTTATCAGTGAACTTGCTCCAGTAGCCTTTCAGGGATTTGAAGCGATTGTCCATCCAGCCCCTGGCCACCACA |
| | | GCTATATCAGACTCCTGCATGCTGCTTAGTGCATCCTGGACCGTCTTGGAGGCTTGTTCCATGTAGCCCTGCATAGAG |
| | | CCCAGCAGCAAGGATCCCTCTCCCTCATCAGGGAAGCTTGGCCAAGGGCGAATTCTGCAGATATCCATCACACTGGCG |
| | | GCCGCTCGAGCATGCATCTAGAGGGCCCAATTC |
| Argininosuccinate lyase | D13978 | CTCTATGACATGATTACGAATTTAATACGACTCACTATAGGGAATTTGGCCCTCGAGGCCAAGAATTCGGCACGAGGCT |
| | | GGTCCACTAACTCCAGGGACTGTGGGACAGAGGAACAAACAAGAAAGAACTCGAGGCCTCGCTGAGTGGCGCGCTTG |
| | | CTGAGTGCTGCCACATCCCGCAAGCTGAGCAGGTATCCCAACTCTGTTCCTGCCCGGTAGACCACCCGAGGTGGTGA |
| | | GTGTGGTCTTGTCTTCCAGATTCGTAGGACAGAAGCTCCAGGAGGAGGACCCGCCCAACATGGCATCGGAGAGCGGG |
| | | AAGCTATGGGGTGGCCGATTTGCAGGCTCGGTCGACCCCACCATGGACAAGTTCAACTCATCTATCGCCTATGACCGG |
| | | CATCTGTGGAATGTGGACCTGCAGGGAAGCAAGGCCTACAGCAGGGGCCTGGAGAAGGCAGGGCTTCTCACCAAAG |
| | | CTGAGATGCAGCAGATACTGCAAGGCCTGGACAAGGTGGCTGAAGAGTGGGCCCAAGGCATCTTCAAATTGTACCCTA |
| | | ATGATGAAGACATCCACACGGCCAACGAGCGGCGCCTGAAGGAACTCATTGGTGAAGCTGCAGGGAAGTTACACACA |
| | | GGCAGAAGTCGCAA |
| Arginosuccinate synthetase 1 | M36708 | TCTATGACATGATTACGAATTTAATACGACTCACTATAGGGAATTTGGCCCTCGAGGCCAAGAATTCGGCACGAGGGTG |
| | | ACAAAGAGGCGCGGGCCTCCCCGCTCTGCAGCTCTCCCAGGCTCCAGCATTAATTGTTGTGATAAATTTGTAATTGTAG |
| | | CTTGTTCTCCTACCACCTGACTGGGGCTGCTGTGCCCCCCCTCACCTCCCCCCCACCCACAGGCTTTGTTCCCTGGTC |
| | | CCCTATAGCCTACAAAAGTGGTCATCGAAGGGAAGGGGGGGTGGCAGGCAGCTGCAGAAAGCGCGTAAAATGACAAT |
| | | TAAAAGAAGTTACATTAGTCTTTCATTTGTCAAAAAAAAAAAAAAAAAAAAAAAANAAAAAAACCNCTTGCGGCCGCAAG |
| | | CTTATTCCCTTTAGTGAGGGTTAATTTTAGCTTGGCACTGGCCGTCGTTTTACAACGTCGTGACTGGGAAAACCCTGGC |
| | | GTTACCCAACTTAATCGCCTTGCAGCACATCCCCCTTTCGCCAGCTGGCGTAATAGCGAAGAGGCCCGCACCGATCGC |
| | | CCTTCCCAACAGTTGCGCAGCCTGAATGGCNAATGGGACNCGCCCTGTAGCGGCGCATTAAGCGCGGCGGGTGTGG |
| | | TGGTTACGCGCANCGTGACGCTACACTTGCCAAGCGCCCTANCC |
| Bax (alpha) | U49729 | CTCAAGTTATGCATCAAGTTTGGTACCGAGTTGGATCCACTAGTAACGGCCGCAGTGTGCTGAATTCGCCCTTCGCGG |
| | | AATTCGGGGCCTTTTTGTTACAGGGTTTCATCCAGGATCGAGCAGAGAGGATGGCTGGGGAGACACCTGAGCTGACCT |
| | | TGGAGCAGCCGCCCCAGGACGCATCCACCAAGAAGCTGAGCGAGTGTCTCAGGCGAATTGGCGATGAACTGGACAAC |
| | | AACATGGAGCTGCAGAGGATGATTGCTGATGTGGATACAGACTCCCCCCGAGAGGTCTTCTTCCGTGTGGCAGCTGAC |
| | | ATGTTTGCAGACGGCAACTTCAACTGGGGCCGGGTGGTTGCCCTTTTCTACTTTGCTAGCAAACTGGTGCTCAAGGCC |
| | | CTGTGCACTAAAGTGCCCGAGCTGATCAGAACCATCATGGGCTGGACACTGGACTTCCTCCGGGAGCGGCTGCTTGT |
| | | CTGGATCCAAGACCAGGGTGGCTGGGATGGCCTCCTTTCCTACTTCGGGACCCCCACATGGCAGACAGTGACCATCTT |
| | | TGTGGCTGGAGTCCTCACTGCCTCGCTCACCATCTGGAAGAAGCTTGGCCAAGGGCGAATTCTGCAGATATCCATCAC |
| | | ACTGGCGGCCGCTCGAGCATGCATCTAGAGGGCCGN |
| Beta-actin | V01217 | TTGNGAATTGGGCCCTCTAGATGCATGCTCGAGCGGCCGCCAGTGTGATGGATATCTGCAGAATTCGCCCTTGCGCG |
| | | GATCCCCGGGACCTGACAGACTACCTCATGAAGATCCTGACTGAAAGGGGCTACAGCTTCACCACCACAGCTGAGAG |
| | | GGAAATCGTGCGTGACATTAAAGAGAAGCTGTGCTATGTTGCCCTAGACTTCGAGCAAGAGATGGCCACTGCCGCATC |
| | | CTCTTCCTCCCTGGAGAAGAGCTATGAGCTGCCTGACGGTCAGGTCATCACTATCGGCAGTGAGCGGTTCCGATGCCC |
| | | CGAGGCTCTCTTCCAGCCTTCCTTCCTGGGTATGGAATCCTGTGGCATCCATGAAACTACATTCAATTCCATCATGAAG |
| | | TGTGACGTTGACATCCGTAAAGACCTCTATGCCAACACAGTGCTGTCTGGTGGCACCACCATGTACCCAGGCATTGCT |
| | | GACAGGATGCAGAAGGAGATTACTGCCCTGGCTCCTAGCACCATGAAGATCAAGATCATTGCTCCTCCTGAGCGAAGC |
| | | TTGGCCAAGGGCGAATTCCAGCACACTGGCGGCCGTTACTAGTGGATCCGAGCTCGGTACCAAGCTTGATGCATAGC |
| | | TTGAGTATTCTATAGTGGCACCTAAATAGCTTGGCGTAATCATGGGCATAGCTGGTTCCTGTGTG |
| Beta-actin, sequence 2 | V01217 | TGACATGATTACGAATGTAATACGACTNCTATAGGGAATTTGGCCCTCGAGGCCTAGAATTCGGNACGAGGCAGGATT |
| | | NAAAAACTGGAACGGTGAAGGCGACCGCAGTTGGTTGGAGCAAACATCCCCCAAAGTTCTACAATGTGGCTGAGGACT |
| | | TTGATTGTACATTGTTTTTTGTTTTTGGTTTTTTTAATAGTCACTCCAAGTATCCACGGCATAGATGGTTACAGGAAGTCC |
| | | CTCACCCTCCCAAAAGCCACCCCCAACTCCTAAGGGGAGGATGGCTGCATCCATGCCCTGAGTCCACACCGGGGAAG |
| | | GTGACAGCATTGCTTCTGTGTAAATTATGTACTGCAAACATTTTTTTAAATCTTCCGCCTTAATACTTCATTTTTGTTTTTA |
| | | ATTTCTGAATGGTCAGCCATTGTGGCCTGCCCCTTTTTTTTGTCCCCCCAACTTGATGTATGAAGGCTTTGGTCTCCCTG |
| | | GGAGTGGTTTGAGGTGTTGAGGCAGCCAGGGCTGGCCTGTACACTGACGTGAGACCGTTTTAATAAAAGTGCACACCT |
| | | TACAAACNAAAAAAAAAAAAAAAAAAAAA |
| Betaine homocysteine methyltransferase | NM_030850 | TCTATGACATGATTACGAATTTAATACGACTCACTATAGGGAATTTGGCCCTCGAGGCCAAGAATTCGGCACGAGGCTT |
| (BHMT) | | CGAATAGCTTCCGGCAGACCGTACAATCCTTCGATGTCCAAGCCGGATGCTTGGGGAGTGACGAAAGGGGCAGCAGA |
| | | GCTGATGCAGCAGAAGGAAGCCACCACTGAGCAGCAGCTGAGAGCGCTCTTCGAAAAACAAAAATTCAAATCCGCACA |
| | | GTAGCCACAGGCCAGCGGTTCGGGGCGAATTCCTCCAGGTCCGGGCCACAGTGTGCACCCGGAAGGAGAAGGCATC |
| | | TCTAAACCAGCGTTTGTGTTGATGCCGGCTTACACCTGTGATTGGTGCTAGTTAGACAAAATGGAATCACAGATAGCAT |
| | | TTCACAGTTACAAAACTACGCTTTAGAATTTTACCTAGAAGGAAGAAAGGAGAAGTCCACAGTAAATCCTGAACACATTT |
| | | CCTACGTGCCTGTCGCATTACAGGCGCACAGGAGTCACTGCAGCGAAGAGAAAGTCACCCGACATCAATCTCATTTCA |
| | | GATAGGGGGATAGGACACCACCCTCCACGAGTGACATAGAACCATTCAGGGACCGTATCATAAGTGACACAGCAACCA |
| | | TCTATATCTAAGATGCTTCCCAAGTGGGATTCCAAGATCTTTTGAGCANGGACCTTANGCAGAAAACA |
| Beta-tubulin, class I | AB011679 | CTCTATGACATGATTACGAATTTAATACGACTCACTATAGGGAATTTGGCCCTCGAGGCCAAGAATTCGGCACGAGGAA |
| | | TTATTACAGTAAACCGTAGCCATGAGGGAAATCGTGCACATCCAGGCCGGACAGTGTGGCAACCAGATCGGTGCTAAG |
| | | TTCTGGGAGGTGATAAGCGATGAACACGGCATCGACCCCACCGGCACCTACCACGGAGACAGCGACTTGCAGCTGGA |
| | | CCGAATCTCTGTGTACTACAATGAAGCTACAGGTGGCAAGTATGTCCCTCGAGCTATCTTAGTGGATCTAGAACCCGG |
| | | GACTATGGACTCCGTTCGCTCAGGTCCTTTTGGCCAGATCTTCAGACCGGACAACTTTGTTTTTGGTCAGTCTGGGGCA |
| | | GGCAACAACTGGGCTAAGGGTCACTACACAGAGGGAGCTGAGCTGGTTGACTCTGTCTTGGATGTGGTGCGGAAGGA |
| | | GGCGGAGAGCTGTGACTGCCTGCAAGGCTTTCAGCTGACCCACTCGCTGGGTGGAGGCACGGGCTCTGGCATGGGC |
| | | ACCCTGCTCATCAGCAAGATTCGAGAAGAATACCCCGACCGCATCATGAACACCTTCAGCGTGGTGCCCTCACCCAAA |
| | | GTGTCTG |
| Biliverdin reductase | M81681 | AACTCAAGCTATGCATCAAGGTTGGGACCGAGCTCGGATCCACTAGTACCGCCCNGCCAGTGTGCTGGAATTCGCCCT |
| | | TCGCGTCTAGATTGGTGTTGGCAGAGCTGGCTCGGTGAGGCTGAGGGACTCGAAGGATCCACGCTCTGCAGCATTCC |
| | | TGAACCTGATTGGATTTGTGTCCAGACGAGAGCTTGGGAGCCTTGATGAAGTACGGCAGATTTCTTTGGAAGATGCTCT |
| | | CCGAAGCCAAGAGATTGATGTCGCCTATATTTGCAGTGAGAGTTCCAGCCATGAAGACTATATACGGCAGTTTCTGCAG |
| | | GCTGGCAAGCATGTCCTCGTGGAATACCCCATGACACTGTCATTTGCGGCGGCCCAGGAGCTGTGGGAGCTGGCCGC |
| | | ACAGAAAGGGAGAGTCCTGCATGAGGAGCAGGTGGAACTCTTGATGGAGGAATTCGAATTCCTGAGAAGAGAAGTGTT |
| | | GGGGAAAGAGCTACTGAAAGGGTCTCTTCGCTTCACAGCTAGCCCACTGGAAGAAGAGAGATTTGGCTTCCCTGCGTT |
| | | CAGCGGCATTTCTCGCCTGACCTGGCTGGTCTCCCTCTTCAAGCTTGGCCAAGGGCGAATTCTGCAGATATCCATCAC |
| | | ACTGGCGGCCGCTCGAGCATGCATCTAGAGGGCCAATTCGCAA |
| C4b-binding protein | NM_012516 | CCTCTAGATGCATGCTCGAGCGGCCGCCAGTGTGATGGATATCTGCAGAATTCGCCCTTGACGAAGTGTCATACACAT |
| | | GTCAAAATGATATAATGCTTACAGCTACTTGCAAGTCAGATGGCACCTGGCATCCCCGGACACCATCATGTCATCAGAG |
| | | TTGTGATTTTCCGCCTGCCATTGCTCACGGACGTTATACAAAATCTTCTTCATACTACGTCAGAACTCAGGTTACATATG |
| | | AATGTGAAGAAGGATACAGACTGGTTGGAGAGGCAACCATCTCCTGCTGGTATTCACAATGGACACCAGCAGCTCCAC |
| | | AGTGTAAAGCTCTATGTCGGAAACCAGAGATAGGAAATGGAGTACTGTCTACCAATAAAGATCAATATGTCGAAACTGA |
| | | AAATGTCACCATCCAATGTGACTCGGGCTTTGTCATGCTAGGTTCCCAAAGCATCACTTGTTCGGAGAATGGAACCCGG |
| | | TACCCAAAGGTGCCCAGATGTGAGCAGGAGGTCCCTAAAGACTGTGAGCACGTGTTNGCAGGCAAGAAGCTCATGCA |
| | | ATGTNTGCAANTTCAAGGGCGAATTCCAGCNCACTGGCGGNCGTTNCTAGTGGATCCGAGCTCGGNCCANCNTGATG |
| | | CATAGCTTGAGTATTNTATANTGCANCTNAATAGCTT |
| Calpactin I heavy chain | NM_019905 | ACATGATTACGAATTTAATACGACTCACTATAGGGAATTTGGCCCTCGAGGCCAAGAATTCGGCACGAGGCTAGGGAG |
| | | GCTCTCTGCAATAGGTGCCCGGCCCAGCTTTTTTTTCAAAATGTCTACTGTCCACGAAATCCTGTGCAAGCTCAGCTTG |
| | | GAGGGTGATCATTCTACACCCCCAAGTGCCTATGGGTCGGTCAAACCCTACACCAACTTCGACGCTGAGAGGGATGCT |
| | | TTGAACATTGAAACAGCAATCAAGACCAAAGGCGTGGACGAGGTCACCATTGTCAACATTCTGACTAACCGCAGCAAT |
| | | GCACAGAGGCAGGACATTGCCTTCGCCTACCAGAGGAGGACCAAAAAGGAACTGCCATCGGCGATGAAGTCGGCCTT |
| | | GTCTGGTCACCTGGAGACCGTGATGTTAGGCCTGTTGAAGACACCTGCTCAGTACGATGCCTCTGAGCTCAAAGCCTC |
| | | CATGAAGGGCCTGGGGACTGATGAGGACTCCCTCATCGAGATCATCTGCTCAAGAACCAACCAGGAGCTGCAGGAGA |
| | | TTAACCGAGTGTATAAGGAAATGTACAAGACCGATCTGGAGAAGTGGCCCTTACCTGTGCCCCAACCTAATGTTCTAGA |
| | | GAATCAGCCTGCCACTAATGGGACCCCTGAACTCCTCCTGGGAANATGACGACAGANCTTGCCN |
| Calpain 2 | NM_017116 | TATGACATGATTACGAATTTAATACGACTCACTATAGGGAATTTGGCCCTCGAGGCCAAGAATTCGGCACGAGGATTGC |
| | | ATAACCCTCGGTAAGGTCAGAGAGAGCTTTATGCCTGTACATAGTATATCCTTTCTACTTCTACACATCGTCTCTTCATA |
| | | GCAATATTAAACCAGGAAGGACTGTTGAGAGGTTTAAGTAATTGGGCAAACAGCAAATTGATCTCAGAGGCCACAATGC |
| | | CTTGATGGGAATATTTAAAGAAATTTAAAAATTTAAGATGCAGTGGTGGCATCTTCCACAGCAGGCCAGAGAGAAACAG |
| | | AGAGAGAGAGAGAGAGACTTGGAAATACAATAATAAAGTAATCTGTGGAGGCGTCTAAAGAATCCAGTCTGATGGAGG |
| | | CATCTAAAGAATCCAGTCTGATGGAGGCATCTAAAGAATCCAGGAGCCAGGAGTATCTGACACAAGGCCAACCTGGGC |
| | | TACCTAGCAAGACCTGATCTCAAAAAATAAATGTCAATTAAAAAAAAAGAAGGCATGTTTCCATCATTTGGTTCTAACAAA |
| | | AGATGGTTTTTATACACCTTTGTGAACAGCAAGCCCTCTTAGACTCAAGTCCTTGACCATATTTGGCCTTTAGACATTGA |
| | | AGTAGTGAGTAGTGAGATCATTTGACTGACCAACTGACCANGTCTGCCATCTGAAAGAAA |
| Carbamyl phosphate synthetase I | M12335 | CTCAACGCCAACAATGTTCCTGCCACCCCAGTGGCTTGCCATCTCAGGAAGGACAGAATCCCAGCCTCTCTTCCATCA |
| | | GAAAGTTGATAAGAGACGGAAGCATTGACCTAGTGATTAACCTCCCCAATAACAACACCAAATTTGTCCATGATAATTAT |
| | | GTGATTCGGAGGACAGCTGTGGACAGTGGAATTGCTCTGCTCACCAATTTCCAGGTGACCAAACTTTTTGCTGAGGCT |
| | | GTGCAGAAAGCTCGTACTGTGGACTCCAAGAGTCTGTTCCACTACAGGCAGTACAGCGCTGGAAAGGCAGCATAGAG |
| | | CAATATGCTGGTCTAGTGAATTCATCCTTCAGTCAGCAGGAGCCACACTGTACCAAAGGGACTGGTCCCAGCCTATCG |
| | | CCAGAAATGGATGTTGTTGCCAAACCTTATTTTTGGTTTCCCTGTTCTTAGTAGTCTGTATCTTTAACCACTGAATTGTTG |
| | | TCAGTCACTGTTTCAAAACCATCANGGTCTTCCCAAGTCTCTTGTCTTCACAAGGAATCCAACCATTCATACATTATCTTT |
| | | TGTANACCGACTTGTACATTTCATGGG |
| Carbonic anhydrase III | M22413 | TGCGAATTGGGCCCTCTAGATGCATGCTCGAGCGGCCGCCAGTGTGATGGATATCTGCAGAATTCGCCCTTCGCGGG |
| | | ATCCAGGTCCATGCTGAGAGGTGGCCCTCTCTCTGGACCCTACCGACTTCGCCAATTCCATCTTCACTGGGGCTCCTC |
| | | GGATGACCATGGCTCTGAGCACACAGTGGATGGAGTGAAGTATGCTGCTGAGCTTCACCTGGTTCACTGGAACCCGAA |
| | | GTATAACACCTTCGGAGAGGCTCTGAAGCAGCCCGATGGGATTGCTGTGGTTGGCATTTTTCTGAAGATAGGACGGGA |
| | | GAAAGGCGAGTTCCAGATTCTCCTTGATGCCCTGGACAAAATTAAGACTAAGGGCAAGGAGGCTCCTTTTAATCACTTC |
| | | GACCCATCGTGCCTGTTCCCTGCTTGCCGGGACTATTGGACCTACCATGGCTCCTTCACCACGCCACCCTGCGAGGAG |
| | | TGCATTGTGTGGCTGCTACTGAAAGAGCCCATGACAGTGAGCTCAGACCAGATGGCCAAGCTGCGCAGCCTGTTCGC |
| | | CAGTGCAGAAAATGAGCCCCCGGTGCCTCTGGTGGGGAATTAAGCTTGGCCAAGGGCGAATTCCAGCACACTGGCGG |
| | | NCGGTACTAGTGGATCCGAGCTCGGACCAAGCTTGATGCATAGCTTGAGTATTCTATAGTG |
| Carbonyl reductase | X84349 | TGCGAATTGGCCCTCTAGATGCATGCTCGAGCGGCCGCCAGTGTGATGGATATCTGCAGAATTCGCCCTTTGACCCCA |
| | | CCCCCTTCCACATTCAAGCAGAGGTGACAATGAAAACCAACTTTTTTGGTACCCAAGATGTCTGCAAGGAGCTACTCCC |
| | | TATAATAAAACCCCAAGGCAGAGTGGTGAATGTATCAAGCAGCGTGAGTCTCAGGGCCCTGAAAAGCTGCAGCCCGG |
| | | AGCTGCAGCAGAAGTTTCGAAGTGAGACCATCACTGAGGAAGAGCTGGTGGGGCTCATGAACAAGTTTGTAGAGGAT |
| | | GCAAAGAAAGGAGTCCATGCGAAAGAAGGCTGGCCCAATAGTGCATATGGGGTCACCAAGATAGGGGTGACAGTCCT |
| | | GTCCAGAATCTATGCCAGGAAACTCACTGAGGAGAGGAGAGAGGACAAGATCCTCCTGAATGCCTGCTGCCCTGGGT |
| | | GGGTCAGAACCGACATGGCAGGACCAAAAGCCACCAAAAGCCCAGAAGAAGGAGCAGAGACCCCCGTGTACTTGGC |
| | | CCTTTTGCCTCCAGGTGCAGAGAAGGGCGAATTCCAGCACACTGGCGGCCGTTACTAGTGGATCCGAGCTCGGTACC |
| | | AAGCTTGATGCATAGCTTGAGTATTCTATAGTGTCACCTAAATAGCTTGGCGTAATCATGGGCATAGCTGGTTNCTGTG |
| | | TGAAAT |
| Carnitine palmitoyl-CoA transferase | L07736 | ANTTGAGAATTGGGCCCTCTAGATGCATGCTCGAGCGGCCGCCAGTGTGATGGATATCTGCAGAATTCGCCCTTGCGC |
| | | GGATCCCCTGCACTATGGAGTCCTGCAACTTTGTGCAGGCCATGATGGACCCCAAGTCAACGGCAGAGCAGAGGCTC |
| | | AAGCTGTTCAAGATAGCTTGTGAGAAGCACCAGCACCTGTACCGCCTCGCCATGACGGGCGCCGGCATCGACCGCCA |
| | | TCTCTTCTGCCTCTATGTGGTGTCCAAGTATCTTGCAGTCGACTCACCTTTCCTGAAGGAGGTATTGTCTGAGCCATGG |
| | | AGGTTGTCTACGAGCCAGACTCCTCAGCAGCAGGTGGAGCTCTTTGAAAGCTTGGCCAAGGGCGAATTCCAGCACACT |
| | | GGCGGCCGTTACTAGTGGATCCGAGCTCGGTACCAAGCTTGATGCATAGCTTGAGTATTCTATAGTGTCACCTAAATAG |
| | | CTTGGCGTAATCATGGTCATAGCTGTTTCCTGTGTGAAATTGTTATCCGCTCACAATTCCACACAACATACGAGCCGGA |
| | | AGCATAAAGTGTAAAGCCTGGGGTGCCTAATGAGTGAGCTAACTCACATTAATTGCGTTGCGCTCACTGCCGCTTTCCA |
| | | GTCGGGAAACCTGTCGNGGCCAGCTTGCATTAATGAATCGGCCAACGCCCCGGGGAGAGGCGGTTTG |
| Caspase 6 | AF025670 | TTGGGAATTGGGCCCTCTAGATGCATGCTCGAGCGGCCGCCAGTGTGATGGATATCTGCAGAATTCGCCCTTCGCGG |
| | | GATCCATGCCGATTGCTTCCTGTGTCTTCCTGAGCCACGGGGAAGGCAACCACATTTACGCATACGATGCCAAAATTG |
| | | AAATTCAGACCTTGACTGGCTTGTTCAAAGGAGACAAGTGTCAGAGCCTGGTTGGAAAACCCAAGATATTCATCATCCA |
| | | GGCCTGTCGGGGCAGCCAGCATGACGTGCCCGTGGTTCCTCTGGACGTGGTGGATCATCAGACAGACAAGCTGGAC |
| | | GACAACGTGACCCAGGTGGATGCTGCCTCGGTGTACACTCTGCCTGCGGGGGCGGACTTCCTCATGTGTTACTCTGTT |
| | | GCAGAAGGGTATTACTCTCACCGAGAGACTGTGAACGGCTCCTGGTACATTCAGGATTTGTGCGAGATGCTGGCCAGG |
| | | CACGGCAGTTCCCTGGAGTTCACCGAGCTGCTCACGCTGGTGAACAGGAAGGTCTCTCAGCGCCGTGTGGATTTCTG |
| | | CAAAGACCCAGGTGCAATTGGCAAGAAGCAAGCTTGGCCAAGGGCGAATTCCAGCACACTGDCGGCCGTTACTAGTG |
| | | GATCCGAGCTCGGTACCAACTTGATGCATACTTGAGTATTCTATAGTGTCACCTAAATAGCTTGCGTAATCATGN |
| Cathepsin B | X82396 | NNAGGGATACCAATTTCACACAGGAACCAGCTATGNCCATGATTACGCCAAGCTATTTAGGTGNCACTATAGAATACTC |
| | | AAGCTATGCATCAAGTTGGTACCGAGCTCGGATCCACTAGTAACGGCCGCCAGTGTGCTGGAATTCGCCCTTTGCAGG |
| | | CAGGGAGTGAGGCAGATAGGAGTCTTTGATTCTTTGAGTTCACCTAACATGCATGAAGCTTTCAGCAAGGTTTGGGTGA |
| | | CTGGACCAGAGCTGCCCCCCATCAGAGCCACCCTACCTGGCTCCCTTCTAGCCTATCCCATGACAGTGATCACAGCCT |
| | | ACATCCTAGCCTCTCCCTAGACTAGTGCTGTGTGTAGCGCCTGCTGCTGTGGCTCTGTCTGCCATCCCTCCCACCCCC |
| | | ACCTCAGCATCCCCAGACCAAGGGCTTTGGCTGCAGGATTTCCAAAGGAATGAACACCAACTACATGATGAGAGGACA |
| | | GATGCCACCCGCCAGCTGCACCTTGAAGCTAGTCACTTCTAGGACTGTGGCAGGCTTTGATGGCCCAATGCAGTTCTC |
| | | TGGAGAAAACTACCTTTCCCCAAGGCATCTGCACCCATTGACAATGGTAATGTGCCCATCTCTCCTTGGTCCTGCCCAA |
| | | GGGCGAATTCTGCAGATATCCATCACACTGGCGGCCGCTCGAGCATGCATCTAGAGGGCCCAATTCGC |
| Cathepsin L, sequence 2 | S85184 | GACCTCGACCATGGGGTTCTGGTGGTTGGCTATGGTTATGAAGGAACAGATTCAAATTCAAATAAGGATAAATACTGGCTTGTCA |
| | | AAAACAGCTGGGGTAAAGAATGGGGTATGGATGGCTACATCAAAATAGCCAAAGACCGGAACAACCACTGCGGACTTG |
| | | CCACCGCAGCCAGCTATCCTATCGTGAATTGATGGACAGCGATAATAAGGACTTACGGACACTACATCCGAAGGAGTT |
| | | CATCTTAAAACTGACCAAACCCGTCTCTGAGTGAGACCATGGTACTTGAATCGTTCAGGATCCAAGTCACGATTTAAATT |
| | | CTGTTGACATTTTTACATGGGTTAAATGTTACCACTACTTAAAACTCCTGTTATAAACAGCTTTATAATATTGGACACTTA |
| | | ATGCTTAATTCTGATTCTGGAATATTTGTTTTATAAAAGTTGNATAAAACTTTCTTTACCTTTTAAAAATTTTAAGCTC |
| | | AGTGCAAAAAAAAAAAAAAAAAAAAAAAAAA |
| Cathepsin S | L03201 | GAATGGGCCCTCTAGATGCATGCTCGAGCGGCCGCCAGTGTGATGGATATCTGCAGAATTCGCCCTTGGTACATTGAG |
| | | CTCCCCTTCGGCGATGAAGAAGCTCTGAAAGAAGCAGTGGCCACTAAAGGGCCTGTCTCTGTGTGCATCGACGCCAG |
| | | CCATTCCTCCTTCTTCCTCTACCAAAGTGGTGTCTATGATGACCCCTCCTGTACCGAGAATGTGAATCATGGTGTTCTC |
| | | GTGGTTGGCTATGGGACTCTTGATGGGAAAGACTACTGGCTTGTGAAAAACAGTTGGGGCCTTCACTTTGGTGATCAA |
| | | GGATATATTCGGATGGCGAGAAATAACAAAAATCACTGCGGGATTGCTAGCTATTGCTCTTACCCAGAAATCTAAACCG |
| | | TTTCTTCTTTTTCTAATGGGTCACAAAGACGGAATACACTCTAATTTAATTTAATTTTGTCTGCTCCATCCGGAGGACCCAAGTG |
| | | TGTCGTGATCAGTGTGTACATACTGTGCTAACTGGCTTACAGCTTGTTTGTTTTATAACCTTACCTCTCTCTGAAAAGTCT |
| | | GTAAGCAAGGACGCGCTGAGGAAGGGCGAATTCCAGCACACTGGCGGGCGTTACTAGTGGATCCGAGCTCGGACCA |
| | | AGCTTGATGCATAGCTTGAGTATTCTATAGTGTCCCTAAATAGCTTGGCGTAATCATGGNCATAGCTGGTTCCTGTGTG |
| | | AAAT |
| Cholesterol 7-alpha-hydroxylase (P450 VII) | J05509 | GAAATTGNGCCCTCTAGATGCATGCTCGAGCGGCCGCCAGTGTGATGGATATCTGCAGAATTCGCCCTTCATAGCTGG |
| | | GGCCAGAGCTTCATCACTTTCAGAAAGCAATGTCCTTTGTATTTATTTTCAAAATGAAGATATTCCAATTGGCAGGATATT |
| | | TTTCCTAAGGAAATTGCTTTATATTTTTATGAAAACTACCAATTAATTATGAAAGGGCTTGAAATTCACGTTTTAGTGAAAT |
| | | TACTGATTTTTCACTAGTAAGGTTCTTCAGGTGTGAAACTGTATTATAAAAATGTTGTAATGGGTCACACTGTGCTTTGCA |
| | | TAAAGGTAAAGGAAACTATGTTTCAGCCTTTTCTGTGTCTATGAGCTTCGAAAATAATCTTACTGTTCTAGAAACACTGG |
| | | GGAGGCTTCGACATGCTCTCGCTATATTTTATTTTACTGGTGCTAGAAATTTTCATTTCAGTTTTCAACTACCTTATCTTT |
| | | CCCCCATTTTGACATGCATGCCAATGAGAAGAGTATTTTTTAGGAATTAACAAGGCACCTCCCAGAACCCTACCCTGAG |
| | | ACTAAGGGCGAATTCCAGCACACTGGCGGCCGTACTAGTGGATCCGAGCTCGGTACCAAGCTTGATGCATAGCTTGA |
| | | GTATTCTATAGTGTCCCTAAATAGCTTGGCGTAATCATGGGCATAGCTGNTTCCTGTGTGAAATTGNTATCCGCTCAC |
| Cholesterol esterase | L46791 | AATACTCAAGCTATGCATCAAGCTTGGTCCGAGTTCGGATCCACTAGAACGGCCGCCAGTGTGTNGAATTCGCCCCTT |
| | | CGCGGGATCTTGGAAGTCCTACCCAACACTGAAAATCTCTGAGAAAATGATTCCAGTGGTTGCTGAGAAGTACTTCGG |
| | | AGGGACAGATGACCCTGCCAAAAGGAAAGACCTGTTCCAGGACTTGGTTGCAGATGTGATATTTGGTGTCCCATCAGT |
| | | GATGGTGTCTCGAAGCCACAGAGATGCTGGAGCCCCCACCTTCATGTATGAATTTGAGTATCGCCCAAGCTTTGTATCA |
| | | GCCATGAGGCCCAAGACAGTGATCGGAGACCATGGTGATGAACTCTTCTCAGTATTTGGATCTCCATTTTTAAAAGATG |
| | | GTGCCTCAGAAGAGGAGACCAATCTCAGCAAAATGGTGATGAAATACTGGGCCAACTTTGCTCGGAATGGGAGCCCTA |
| | | ATGGGGGAGGGCTGCCCCATTGGCCAGAATATGACCAGAAGGAAGGGTACCTGAAGATTGGTGCCTCAACTCAGGCA |
| | | GCCCAGAGGCTGAAGGACAAAGAAGTGGCTTTTTGGTCTGAGCTCACTGCAGGGCCAAGGGCGAATTCTGCAGATAT |
| | | CCATCACACTGGCGGCCGCTCGAGCATGCATCTAGAGGGCCCAATTCT |
| Choline kinase | D10262 | GCGAATTGGGCCCTCTAGATGCATGCTCGAGCGGCCGCCAGTGTGATGGATATCTGCAGAATTCGCCCTTCTCGAGCT |
| | | TGAGAACCTGAGGTCATTGCTGCAGTATACTAGATCCCCAGTTGTTTTGTCATAATGACTGTCAAGAAGGTAATATCT |
| | | TATTGTTGGAAGGCCAAGAGAATTCTGAAAAGCAGAAGTTGATGCTCATTGACTTTGAATACAGCAGTTACAATTACAG |
| | | GGGATTTGACATTGGAAATCATTTCTGTGAATGGATGTATGATTATACCTATGAAAAGTATCCTTTCTTCAGAGCAAACA |
| | | TTCAGAAGTATCCTACCCGAAAACAACAGCTCCATTTTATTTCAAGTTACTTGACTACATTCCAAAATGATTTTGAAAGCC |
| | | TCAGCAGTGAAGAGCAGTCTGCTACAAAAGAAGACATGTTGCTTGAAGTCAACAGATTTGCCCTTGCCTCTCATTTCCT |
| | | CTGGGGACTTTGGTCCATTGTACAGGCCAAGATCTCATCCATTGAATTTGGGTACATGGAATATGCCCAAGCCAGGTTC |
| | | GATGCCTACTTTGACAAGGGCGAATTCCAGCACACTGGCGGCCGTTACTAGTGGATCCGAGCTCGGTACCAAGCTTGA |
| | | TGCATAGCTTGAGTATTCTATAGGNCACCTAAATAGCTTGGCGTAATCATGGNCATAGCTGN |
| c-H-ras | M13011 | CCTCTAGATGCATGCTCGAGCGGCCGCCAGTGTGATGGATATCTGCAGAATTCGCCCTTATCCAGCTGATCCAGAACC |
| | | ATTTTGTGGACGAGTATGATCCCACTATAGAGGACTCCTACCGGAAACAGGTAGTCATTGATGGGGAGACGTGTTTACT |
| | | GGACATCTTAGACACAGCAGGTCAAGAAGAGTATAGTGCCATGCGGGACCAGTACATGCGCACAGGGGAGGGCTTCC |
| | | TCTGTGTATTTGCCATCAACAACACCAAGTCCTTTGAAGACATCCATCAGTACAGGGAGCAGATCAAGCGGGTGAAAGA |
| | | TTCAGATGATGTGCCAATGGTGCTGGTGGGCAACAAGTGTGACCTGGCCGCTCGCACTGTTGAGTCTCGGCAGGCCC |
| | | AGGACCTTGCCCGCAGCTATGGCATCCCCTACATTGAAACATCAGCCAAGACCCGGCAGGGTGTGGAGGATGCCTTC |
| | | TACACACTAGTACGTGAGATTCGGCAGCATAAACTGCGGAAACTGAACCCGCCTGATGAGAGTGGCCCTGGCTGCATG |
| | | AGCTGCAAGTGTGTGCTGTAAGGGCGAATTCCAGCACACTGGCGGCCGTTACTAGTGGATCCGAGCTCGGTACCAAG |
| | | CTTGATGCATAGCTTGAGTATTCTATAGTGTCACCTAAATAGCTTGGCGT |
| c-jun | X17163 | GCGAATTGGGCCCTCTAGATGCATGCTCGAGCGGCCGGCAGTGTGATGGATATCTGCAGAATTCGCCCTTATCGCGG |
| | | GATCCTCGTTCCTCCAGTCCGAGAGTGGCGCCTACGGCTACAGTAACCCTAAGATTCTGAAGCAGAGCATGACCTTGA |
| | | CCCTGGCCGACCCGGTGGGCAATCTGAAGCCGCACCTCCGAGCCAAGAACTCGGACCTTCTCACGTCGCCCGACGTC |
| | | GGGCTGCTCAAGCTGGCGTCGCCGGAGCTGGAGCGCCTGATCATCCAGTCCAGCAATGGGCACATCACCACTACACC |
| | | GACCCCCACTCAGTTCTTGTGCCCCAAGAACGTGACCGACGAGCAGGAGGGCTTCGCCGAAGGCTTCGTGCGCGCCC |
| | | TAGCTGAACTGCATAGCCAGAATACGCTGCCCAGTGTCACCTCCGCGGCACAACCTGTCAGTGGGGCGGGCATGGTC |
| | | GCTCCCGCTGTGGCCTCAGTAGCTGGCGCTGGCGGCGGCGGCGGCTACAGCGCCAGCCTGCACAGTGAGCCTCCG |
| | | GTCTACGCCAACCTCAGCAAAGCTTGGCCAAGGGCGAATTCCAGCACACTGGCGGCCGTTACTAGTGGATCCGAGCT |
| | | CGGTACCAAACTTGATGCATAGCTTGAGTATTCTATAGTGTCACCTAA |
| c-myc | X01023 | NTNCNNANGNGCCCTNTANATGCTGCTCGAGCGGCCGCCAGTGTGATGGATATCTGCAGAATTCGCCCTTCGCGGGA |
| | | TCCTGGAGAAGAGGCAAACCCCTGCCAAGAGGTCCGAGTCAGGGTCATCCCCATCAAGAGGCCACAGCAAACCTCCA |
| | | CACAGCCCACTGGTCCTCAAGAGGTGCCATGTCTCTACTCACCAGCACAATTATGCAGCACCCCCCTCCACAAGGAAG |
| | | GACTATCCGGCTGCCAGGAGGGCCAAGTTGGACAGTGGCAGGGTCCTGAAACAGATCAGCAACAACCGCAAATGCTC |
| | | CAGCCCCAGGTCCTCAGACACCGAGGAAAACGACAAGAGGCGGACACACAACGTCTTGGAACGTCAGAGGAGAAACG |
| | | AGCTGAAGCGTAGCTTTTTTGCCCTGCGCGACCAGATCCCTGAGTTGGAAAACAACGAAAAGGCCCCCAAGGTAGTTA |
| | | TCCTCAAAAAAGCCACCGCCTACATCCTGTCCGTTCAAGCAGATGAGCACAAACTCATCTCAGAAAAGGACTTACTGAG |
| | | GAAACGACGAGAACAGTTGAAACACAAAAGCTTGGCCAAGGGCGAATTCCAGCACACTGGCGGGCCGGTACTAGTGG |
| | | ATCCGAGCTCGGTCCAAACTTGATGCATTAGCTTGAGTATTCTATAGTGTCACCTAAATAGCTTGGGGTA |
| Cofilin | NM_007687 | TATGACATGATTACGAATTTAATACGACTCACTATAGGGAATTTGGCCCTCGAGGCCAAGAATTCGGCACGAGGCGGAT |
| | | CCCGGCAGCAGCTGCGGCGGCTCTGTTCTTCTGTAGCTCTTTTCTGTCTCCCTTTCGTTTCCGGAAACATGGCCTCTG |
| | | GTGTGGCTGTCTCTGATGGAGTCATCAAGGTGTTCAATGACATGAAAGTTCGCAAGTCTTCAACGCCAGAAGAAGTGA |
| | | AGAAACGCAAGAAGGCAGTGCTCTTTTGCCTGAGTGAGGACAAGAAGAACATCCCTCCCACCCCCTGGACCGTCCTTT |
| | | TCCCTCCATCCCTGACGGTTCTGGCCTTCCCAAACTGCTTTTGATCTTCTGATTCCTCTTGGGTTGAAGCAGACCAAGT |
| | | CCCGTCCTAGGCACCCAGTTTGGGGGGAGCCTGTATTTTTTTTTTTTAACGACACCCCTACTCCTCATCTGTCCCATCC |
| | | CATGCTGCCAACTTCTAACCACAATAGTGACTCTGTGCTTGTCTGGTTAGTTCTGTGTGTAAATGAACTGTGGAAATGAC |
| | | CCTCCCCTGCACCAGCTGGGTGCCTCCCCTTTTCCTTGATCTTGGGCACTCATGGAAACAGGACCAGTAANGGACCTT |
| | | CAATTAAAAAAAAAAAAAAAAAAAAAAAATTGCGGGCGCNAAGCTTATTCCCTTTANNGANGGGTAAATTTAACTTGGCN |
| | | CTGGGCCN |
| Collagen type II | L48440 | TGCGAATTGGCCCTCTAGATGCATGCTCGAGCGGCCGCCAGTGTGATGGATATCTGCAGAATTCGCCCTTATCGCGGG |
| | | ATCCGGAGCAGCAAGAGCAAGGAGAAGAAGCACATCTGGTTTGGAGAGACCATGAACGGCGGCTTCCACTTCAGCTA |
| | | CGGCGACGGCAACCTGGCTCCCAACACCGCTAACGTCCAGATGACTTTCCTCCGTCTACTGTCCACTGAGGGCTCCCA |
| | | GAACATCACCTACCACTGTAAGAACAGCATTGCCTACCTGGACGAAGCAGCCGGCAACCTCAAGAAGGCCTTGCTCAT |
| | | CCAGGGCTCCAATGATGTGGAGATGAGGGCCGAGGGCAACAGCAGGTTCACGTACACTGCCCTGAAGGATGGCTGC |
| | | ACGAAACACACCGGTAAGTGGGGCAAGACCATCATCGAGTACCGATCACAGAAGACCTCACGCCTTCCCATTGTTGAC |
| | | ATTGCACCCATGGACATCGGAGGGCCTGATCAGGAATTTGGTGTGGACATAGGGCCTGTCTGTTTCTTGTAAAACCCT |
| | | CAACCCCAAAACAACAAAGCTTGGCCAAAGGGCGAATTCCAGCACACTGGCGGCCGTTACTAGTGGATCCGAGCTCG |
| | | GTACCAAGCTTGATGCATAGCTTGAGTATTCTATAGTGTCACCTAAATAGCTTGGCGTAAT |
| Connexin-32 | X04070 | TGCGAATTGGCCCTCTAGATGCATGCTCGAGCGGCCGCCAGTGTGATGGATATCTGCAGAATTCGCCCTTCAGGCCTC |
| | | TGCCTCCTAGGGATTACTCCATCAAACCTTCCCTCCCTCCCTACTCCCCTTCCTCAGAGAGTCTTCTGTCAAAGACCTG |
| | | CCGGCTTGGGAGTGGGGAGCCACTTCTGCACCAGGGCTCAAGGTTATTGAGGGTGTGGGCAATTCTTTCTGCCTATAC |
| | | CCTTTCCTCTTCCCTCTCCCTGAGATGAGGGATGAGATGTTCTGAAGGTGTTTCCAATTAGGAAACTAATCTTAACCCCC |
| | | ATGCTGTCAGGTACCCCACTTTGGGAGTCATGTCAAGTGGGGAGGGCTGTGAGCAAGCAGAGTGGAGGAGGGGCTCT |
| | | GCACTGTGGATGGAGAAGGGAGGGGAGCTTGCCTTGCTGCCTGCTACAAGGAAAANGANGACACATCTANGGTGGG |
| | | GGAAGTTCTTGGAGGGAGAAACAGGCAGATAAATCAAAATGGGGGTTGGNCAAGGGCTTGCCCCCATCCCCCCCCAA |
| | | ANTTTTCCCCCAAAAGGGGGCCCTTNTTTTTTTTTTTTTTTGGAAAAAAATGGGTNAAAGGGGGCGGAAAATTTCN |
| Contrapsin-like protease inhibitor (CPi-21) | D00751 | TGACATGATTACGAATNTAATACGACTCACTATAGGGAATTTGGCCCTCGAGGCCAAGAATTCGGCACGAGGCAGACT |
| | | ACAACCTGGAGGAGGTCCTTCCAGAGCTGGGAATTAGGAAAATCTTCTCCCAGCAAGCTGATCTGAGTAGGATCACAG |
| | | GGACCAAGAACCTGCATGTCTCTCAGGTGGTCCACAAAGCTGTGCTGGATGTGGATGAGACAGGCACAGAAGGAGCC |
| | | GCTGCCACAGCAGTCACAGCAGCCCTAAAAAGTTTACCGCAAACTATACCTCTTCTGAATTTCAACCGGCCATTCATGC |
| | | TGGTTATCACTGACAATAATGGTCAGTCTGTCTTCTTTATGGGCAAAGTCACTAACCCCATGTGAGTCTGAAGCTCCCC |
| | | AAAATCTGACAATTCTGCCCAGGATCCTGGAACAGAGCCTGGATGCTGATCTCTGTATATGCCCTGACATACATGCTCT |
| | | GATTGGCTATTGCAAAGTTGGCTTAGACAGTGACATCAACTATCTCTATGGCTCCCATGTGCACTGGAGCCTTTGGATT |
| | | GTCAAGTGTCAGGCACTTANGACCCTTGGGAGCATCTACACATGTTTCTGAACTTGGAATCTTTCTTTATTCTTCTTNCC |
| | | TGGTGACTCCTCTTTCTGTGTTCATACCCCAAACCAAG |
| C-reactive protein | M83176 | TGGCGAATTGGGCCCTCTAGATGCATGCTCGAGCGGCCGCCAGTGTGATGGATATCTGCAGAATTCGCCCTTCGCGG |
| | | GATCCCAAAGAAGCCACTGGAAGCCTTCACTGTGTGTCTCTATGCGCACGCTGATGTGAGCCGAAGCTTCAGCATCTT |
| | | CTCTTACGCTACCAAGACGAGCTTTAACGAGATTCTTCTGTTTTGGACTAGGAGTCAAGGGTTTAGTATTGCAGTAGGT |
| | | GGGCCTGAAATACTGTTCAGTGCTTCAGAAATTCCTGAGGTACCAACACACATCTGTGCCACCTGGGAGTCTACTACAG |
| | | GAGTTGTAGAGCTTTGGCTTGACGGGAAACCCAGGGTGCGGAAAAGTCTGCAGAAGGGCTACATTGTGGGGACAAAT |
| | | GCAAGCATCATCTTGGGGCAGGAGCAGGACTCGTATGGCGGTGGCTTTGACGCGAATCAGTCTTTGGTGGGAGACAT |
| | | TGGAGATGTGAACATGTGGGACTTTGTGCTATCTCCAGAACAGATCAATGCAGTCTATGTTGGTAGGGTATTCAGCCCC |
| | | AATATTTTGAACTGGCGGGCACTGAAGTACTCGAGGGCCAAGGGCGAATTCCAGCACACTGGCGGGCGTACTAATGG |
| | | ATCCGAGCTCGGACCAAGCTTGATGCATAACTTGA |
| Cyclin D1 | D14014 | GCCCTCTAGATGCATGCTCNAGCGGCCGCCAGNGTGATGGATATCTGCAGAATTCGCCCTTCGCGGAATTCCTGCTG |
| | | GGGACATCCTGTCATGCTGGGCCTTCATTTGATCTGGGACATAGCATCACAGCAGTCAGGGCAACTGTGTTCTGTTAG |
| | | TTATCAATATTGTTACTTGTAGCGGCCTGTTGTGCATGCCACCATGCTGCTGGACCCGGAGAGATTTGTTCTGAGTCTC |
| | | TGGTGCATCATTTAATCTGTTAGGTTCTAGTGTTCTGTCTTGTTTTGTGTTACTCACAGCATTGTGCTAATGTAAAGCCAG |
| | | CCGCAATGCTGTAGGCCCCAGGTTCCCTAGCAAGCTGCCAAACCAAAAGGGTCACCACCAGCTTAGCTGAGGCGTCC |
| | | CAACCAGGCAGGACCCTTGAGGGCTGCTGTGTCCATGGTGATGGGGTGAAGTTTTGGCCAAAGGGCCAAAGGCTGGT |
| | | GGATCCACACAGTCTGCCCTGTGACATGAATGGCTTTGAGGGGCTCTGGCTGGTGGTCAGGTTGGCTTTTGTGTATTC |
| | | TGGTTGACACACCATGGCAAGCTTGGCCAAGGGCGAATTCCAGCACACTGGCGGGCCGNTACTAAGTGGATCCGAAC |
| | | TTCGGTACCAAGCTTGATGCATAGCTTTGAGTATTCTATAGTGNCACCTAAATAGCTTGGCGTAATCATGGCATAACTTG |
| | | TT |
| Cyclin D3 | D16309 | GCGAATTGGGCCCTCTAGATGCATGCTCGAGCGGCCGCCAGTGTGATGGATATCTGCAGAATTCGCCCTTATCGCGG |
| | | GATCCCGCCCCAGTACCTTCCAAAGGTGTTGTTGCCCCTCGCAGGGTCACTGCATTTGGATCTGGGTCCTTCAGAAAT |
| | | CCCGATAGACGCCTATGAGGAGCCAACCTAGATGGCTGCTGTGTAATCCCTACTCCAGCTGCTCTTAGCGGGAACAGC |
| | | CTAGGCCTTGGCTAGAAGAGCAAGCGCCCGTAAACTGTTGCTTTGCTTCCTGCTATGCTTCTGTGGTTGAGGGTCTTG |
| | | AGGGTGCTGATGGTCATTTTAATTTATTGCTTTGAATACAACCGTAAGAGGGTACAGTGAGGCCTGTACCCCACAAGTG |
| | | GTGGTAACCCTGGCGGTTGCTCTTTCCCTCCCCTCTGCTACCGCTTTGTGGCCCAGGAGCCGCTACAGCCTGGGAGG |
| | | GGGTCCTGCCTTCCTCTCCGTAGCCCTCCAGCTCATCTTCAGCGGGGAGGGTTTAGGGATGGATGCCCGTGGAG |
| | | GTGACTGGACTATCCGGAGAGAGGGCGAGAAGCTTGGCCAAAAGGGGCGAATTCNANCACACTGGCGGCCGTTACTA |
| | | GT |
| Cyclin dependent kinase 4 | L11007 | TTGGAATTGGGCCCTCTAGATGCATGCTCGAGCGGCCGCCAGTGTGATGGATATCTGCAGAATTCGCCCTTCGCGGG |
| | | ATCCAGGATCTGATGCGCCAGTTTCTAAGCGGCCTAGATTTCCTTCATGCAAACTGCATTGTTCACCGGGACCTGAAGC |
| | | CAGAGAACATTCTAGTGACAAGTAATGGGACAGTTAAGCTGGCCGACTTTGACCTAGCCAGAATCTACAGCTACCAGAT |
| | | GGCCCTCACGCCTGTGGTTGTTACGCTCTGGTACCGGGCTCCTGAAGTTCTTCTGCAGTCTACATATGCAACGCCTGT |
| | | GGATATGTGGAGTGTTGGCTGTATCTTCGCAGAGATGTTTCGCCGGAAGCCTCTCTTCTGTGGGAACTCTGAGGCTGA |
| | | CCAGCTGGGCAAAATCTTTGATCTCATTGGATTGCCTCCAGAAGACGACTGGCCTCGAGAGGTCTCTCTTCCTCGAGG |
| | | AGCCTTTTCCCCCAGAGGACCTCGGCCAGTGCAGTCAGTGGTGCCGGAGATGGAGGAATCTGGAGCGCAGTTGCTGC |
| | | TGGAAATGCTGACCTTTAATCCACTTAAGCGAAGCTTGGGCAAGGGCGAATTCCAACACACTGGCGGGCGGTACTAAT |
| | | GGATCCGAGCTCGNACCAACTTGATGCATAGCTTGAGTN |
| Cyclin G | X70871 | GNNTTCCNNGGGGATGGATATNTGCAGAATTCGCCNNNATCCNCGGGATCCNATGGCCTCAGGAATNACNGCAAGAC |
| | | TCCCAGGTCTTNTNAAAGNCAGAGATTTCCGGAGNCCTAACTCAGTNATGGGCTTNGACACANAAACATTTTCCCTTTG |
| | | CTGTGAATTTTACTGCAGAGATTGGTTGTCTAAAATAAGGACAGGNGNAGCATCTCGGATGTGTCGGCCTTGAGNNGT |
| | | NTTATTTGGCTGTGGNAATNNATTNAAGAGGNAAGGNANTGTCCCNGCTGGCNNACTTNNTNTGNTCCGNATNAGNCN |
| | | GTANNGGTTCNCAGTTTCAGACGTGATNAGAATGCAAAAAGATTGTGTTNNANANAGTGTGNTGCANAGTCNAAGCTAT |
| | | TACNNCCTTCCANTTTCTNCAGNTCTATCANTCCCTCATTNGGGAGCCCNTGCCATTTGAAAGGAGNNACNNTNNGAAT |
| | | TNTGANAGNGTACNAGCCCTNNTNAAGGNGTGCCACNGCNGGATCATATTTTCTAAGNCAAAGCCNTCTGNGATGGNN |
| | | CTNGCGATCATTNNTTTGGAGATCCAANCNGTNCAGTATGTGNAGNNCACNGAAGGAGNANNATGTNTTCAAGCNCAT |
| | | TCCCAGATAAGTGGCCNGTATNTNNCCTTCTGGCAAGAGCCNGNNTCCTAGTGTCTTCCGAGATCTTCNTATCCGAGT |
| | | GGCGCGNNGNTNNNNANNNNNNC |
| Cystatin C | X16957 | TATGACATGATTACGAATTTAATACGACTCACTATAGGGAATTTGGCCCTCGAGGCCAAGAATTCGGCACGAGGGCGG |
| | | AACCATGGCCAGCCCGCTGCGCTCCTTGATGCTACTGCTGGCCGTCCTGGCCGTGGCCTGGGCCGGAACCTCCAGG |
| | | CCACCCCCGCGATTGTTGGGAGCTCCGCAGGAGGCAGATGCCAGCGAGGAGGGCGTGCAGCGAGCGTTGGACTTCG |
| | | CCGTAAGCGAGTACAACAAGGGCAGCAACGATGCGTACCACAGCCGCGCCATACAGGTGGTGAGAGCTCGTAAGCAG |
| | | CTTGTGGCTGGAATAAACTATTATTTGGATGTGGAGATGGGCCGAACTACATGTACCAAGTCCCAGACAAATTTGACTA |
| | | ACTGTCCTTTCCACGACCAGCCCCATCTGATGAGGAAGGCACTCTGCTCCTTCCAGATCTACAGCGTGCCCTGGAAAG |
| | | GCACACACACCCTGACAAAATCCAGCTGCAAAAATGCCTAAGAGCTGAGTCTCATAGGACCATGCCAATGGTCCCTTA |
| | | CTTGTTCCCCTACCCTGTAGTGTTTTATCCCTGAAAAGGGTGCTCCAGCTCTGGAGGGCATCTNCGGGGTGTTCCCAC |
| | | CAGGAGAGAGTAAAGAAACTGCTGCAGGCAGGTTCTGCACGTCAGAACAGCTGTCCCCTGGTTCTCTTCTCCTTGCAG |
| | | TAN |
| Cyto-chrome P450 1A1 | X00469 | TCTGGTCCTCAGCATCTCCAGGCTTAGACTGTCCTGGATGCTCACCAGAC |
| Cyto-chrome P450 2C11 | U33173 | ACTCTCTAAGCTCTCATCTGTAATGTCTCTTCTGAGGGTCCTGTCTACTT |
| Cytochrome P450 2C23 | X55446 | TATGACATGATTACGAATTTAATACGACTCACTATAGGGAATTTGGCCCTCGAGGCCAAGAATTCGGCACGAGGGCCAA |
| | | AGTTCATGAGGAACTTGACCGTGTGATTGGACGCCACCAACCCCCCAGCATGAAGGACAAGATGAAGCTGCCTTATAC |
| | | CGATGCTGTATTGCATGAGATTCAAAGATACATCACTCTCCTTCCTTCCAGTCTGCCCCATGCTGTGGTCCAGGACACA |
| | | AAATTCAGAGACTATGTCATCCCCAAGGGTACTACTGTACTCCCGATGCTGTCTTCCGTCATGCTGGATCAAAAGGAGT |
| | | TTGCCAACCCAGAGAAGTTTGATCCAGGACACTTTCTGGATAAAAATGGCTGCTTCAAGAAGACAGACTACTTTGTTCC |
| | | CTTCTCCCTTGGAAAGCGGGCCTGTGTTGGTGAGAGTTTGGCCCGGATGGAGCTCTTCCTGTTCTTCACCACCCTTCT |
| | | GCAAAAGTTTTCCCTGAAGACTCTGGTGGAGCCCAAGGACCTTGACATCAAGCCTATTACTACCGGGATTATCAATTTG |
| | | CCGCCACCTTACAAGCTGTGCCTTGTTCCTAGATAAGGGTTTATCCTTCTAATCATTCAAATCAAAGTAATTGGTTCCTTT |
| | | TTCTATAAATATTCTTTTTATGCTAAGCACTGNCTANGGCAGAAACATCATGCATG |
| Cytochrome P450 2D18 | U48220 | TCTCTATGACATGATTACGAATTTAATACGACTCACTATAGGGAATTTGGCCCTCGAGGCCAAGAATTCGGCACGAGGG |
| | | TGATCGCGTCCCTCCTCTTTGCCTGCCGCTTTGAATACAATGACCCACGCTTCATCAGGCTCCTGGACTTGCTGAAGGA |
| | | CACTCTTGAGGAGGAATCTGGATTCCTGCCCATGCTCCTGAATGTGTTCCCGATGCTCCTACACATCCCAGGGCTTCTT |
| | | GGCAAGGTATTCTCTGGAAAGAAGGCCTTCGTTGCCATGCTGGACGAGCTGCTAACTGAACACAAGGTGACCTGGGA |
| | | CCTGCGCAGCCACCCCGAGATCTGACTGATGCCTTCCTGGCTGAGGTGGAGAAGGCCAAGGGGAATCCTGAGAGCA |
| | | GCTTCAATGATGAGAACCTGCGTGTGGTGGTGGCTGACCTGTTCATGGCGGGGATGGTGACCACCTCCACCACACTG |
| | | ACCTGGGCCCTGCTGTTCATGATCCTGCATCCAGATGTGCAGTGCCGAGTACAACAGGAAATCGATGAGGTCATAGGG |
| | | CAGGTGCGGCGTCCAGAGATGGCAGACCAGGCACGAATGCCGTTCACCAATGCTGTCATCCATGANGTGCACGCTTT |
| | | GCAGACATTCTCCCTCTTGGTGTGCCTCACAAAAACTTCTCGTGGCATTTGAATGCANGGCTTTCTTTATCCTAANGGG |
| Cyto-chrome P450 2E1 | M20131 | TTGACCCTAAGGATATCGACCTCAGTCCTGTCACAGTTGGCTTTGGCAGT |
| DNA polymerase beta | M13961 | GGANTGNCCTNTAGATGCTGCTCNAGCGGCCGCCAGNGTGATGGATATCTGCAGAATTCGCCCTTCGCGGAATTCTG |
| | | GATCCCGAGTACATCGCTACAGTCTGCGGCAGTTTCCGAAGAGGCGCAGAGTCCAGTGGAGATATGGACGTTCTGCT |
| | | GACCCACCCAAACTTCACGTCAGAATCAAGCAAACAGCCAAAGTTGTTACATCGTGTTGTGGAACAGTTACAAAAAGTC |
| | | CGTTTCATTACAGATACTCTGTCAAAGGGTGAGACAAAGTTCATGGGTGTTTGCCAGCTTCCCAGCGAGAATGATGAAA |
| | | ACGAATATCCACACAGGAGAATCGATATCAGGTTGATCCCCAAAGATCAGTACTACTGTGGTGTTCTCTACTTCACTGG |
| | | AAGTGACATCTTTAATAAGAATATGAGAGCGCATGCCCTGGAAAAGGGCTTCACAATCAATGAGTACACCATCCGCCCC |
| | | CTGGGGGTCACTGGGGTTGCTGGGGAGCCCCTTCCGGTGGACAGCGAGCAGGACATTTTCGATTACATCCAGTGGCG |
| | | CTCCGGGAGCCCAAGGACAGGAGTGAAAAGCTTGGGNCAAGGGCAAATTCCAGCACACTGGCGGCGGTACTAGTGG |
| | | ATCCGAGCTCGGTCCAAACCTTGATGCATAGCTTGAGTATTCTATAGTGNCACCTAAATAGCTTGGCGTAATCATGGTC |
| | | ATAGG |
| DNA topoisomerase I | AF140782 | AATTGGCCCTCTAGATGCATGCTCGAGCGGCCGCCAGTGTGATGGATATCTGCAGAATTCGCCCTTATCGCGGGATCC |
| | | TGCAGCAGCAGTTTAAAGAGCTCACAGCCCCTGATGAGAATGTACCAGCAAAGATTCTATCTTATAACCGTGCCAATCG |
| | | CGCTGTTGCAATTCTTTGTAACCACCAGAGGGCGCCACCAAAGACCTTTGAGAAGTCAATGATGAACTTGCAGTCTAAG |
| | | ATTGATGCCAAGAAAGATCAGCTAGCAGATGCTCGAAAGGACCTGAAAAGCGCTAAGGCTGATGCCAAAGTCATGAAG |
| | | GATGCAAAGACCAAGAAGGTAGTAGAGTCAAAAAAGAAGGCTGTACAGAGATTAGAAGAGCAGCTGATGAAGCTGGA |
| | | GGTTCAAGCCACAGACCGAGAGGAGAACAAACAAATTGCCTTGGGGACCTCCAAACTCAATTACCTGGACCCTAGGAT |
| | | CACAGTGGCTTGGTGCAAAAAATGGGGGGTCCCAATCGAGAAGATTTACAACAAAACCCAGAGAGAGAAGTTTGCTTG |
| | | GGCCATTGATAAGCTTGGCCAAAAGGGCGAATTCCAGCACACTGGCGGCCGNTCTAGTGGATCCGAACTCGGTCCAA |
| | | ACTTGATGCATACTTGAGTATTCTATAA |
| Dynamin-1 (D100) | X54531 | TATGACATGATTACGAATTTAATACGACTCACTATAGGGAATTTGGCCCTCGAGGCCAAGAATTCGGCACGAGGGAAGT |
| | | ACATGCTGTCTGTGGATAATCTGAAGCTGCGTGATGTGGAGAAGGGCTTCATGTCAAGCAAGCATATTTTTGCCCTCTT |
| | | CAACACTGAGCAGAGGAATGTCTACAAGGATTACCGGCAGCTGGAACTGGCCTGTGAGACACAGGAGGAGGTGGACA |
| | | GTTGGAAGGCTTCCTTTCTGAGGGCTGGCGTGTACCCTGAGCGTGTTGGGGACAAGGAGAAAGCCAGTGAGACTGAG |
| | | GAGAACGGCTCTGACAGCTTCATGCACTCCATGGACAGCTGTTCTGTGACTTGACAGTGGCTCCCCCGACCCCAAAGC |
| | | GTCCTATTCATCATCTGTGACTTAGTCTGTTGTAGTGGTGAGCTGATACATCCAAATGTGATGTTGGTGAAAACTTGTGC |
| | | CCCTCTGTGGTATTGCTCCTGCGACATTCTATAAATATCTATAAATATACCTATATATACATATATATACATATATATGGCT |
| | | GACACAGCCTCGCCTGTAGTGTCAGAATCAACCACCATGCTGTCCTTGTGGAGTCCTGTGGTCCAACTACAANGGAAC |
| | | GCTGTCATCCTGACATCCCCCCTCCAATGTGCCACCTCCAGTGAGCCTCCCTGTCCCTTGGNCTGTGGACAGCCATCC |
| | | CCTTGGCCATCCCCAC |
| Elongation factor-1 alpha | X61043 | CACTATAGAATACTCAAGCTATGCATCAAGCTTGGNCCGAGCTCGGATCCACTAGTACCGGCCGCCAGTGTGTTGGAA |
| | | TTCGCCCTTTGAAGCTTTTGAGTGAAGCTCTGCCTGGGGACAATGTAGGCTTCAACGTAAAGAACGTGTCTGTCAAAGA |
| | | CGTTAGACGTGGCAATGTTGCTGGGGACAGCAAAAATGACCCACCAATGGAAGCAGCTGGCTTCACTGCTCAGGTGAT |
| | | TATCCTGAACCATCCAGGCCAGATCAGTGCTGGCTATGCCCCTGTTCTGGACTGCCACACGGCCCACATAGCATGCAA |
| | | GTTGCCGAGCTTAAAGAGAAGATCGATCGTCGTTCTGGTAAGAAGCTGGAAGATGGCCCCAAATTCTTGAAGTCTGG |
| | | TGATGCTGCCATTGTTGACATGGTCCCTGGCAAGCCCATGTGTGTTGAAAGCTTCTCTGACTACCCTCCACTTGGTCGT |
| | | TTTGCTGTTCGTGACATGAGGCAGACAGTTGCTGTGGGTGTCATCAAAGCCGTGGACAAGAAGGCTGCAGGAGCTGG |
| | | CAAAGTCACCAAGTCTGCCCAGAAAGCTCAGAAGGCTAAATGAATATTATCCCTAACACCTGCCACCCCAGTCTTAATC |
| | | AAAGGGCGAATTCTGCAGATATCCATCACACTGGCGGCCGCTCGAGCATGCATCTAGAGGGCCCAATTCGC |
| Endogenous retroviral sequence, 5′ and | D90005 | TTATGACATGATTACGAATTTAATACGACTCACTATAGGGAATTTGGCCCTCGAGGCCAAGAATTCGGCACGAGGCACT |
| 3′ LTR | | GACAGAAGCACATGGGTGGAGGAAAGACCTCTGCGACTGGCTGATTGATCCCTGCTGAAAGCCGAGGACCTTGTCCA |
| | | CAGACAGGAACAGTTCTCTTCATGAATGAAGGTCAGAGACAAGTGGGTGCTGCCATGGTGGACAACACAATGTCATCT |
| | | AGAATGGCTGAACCTCTACCCCCCCAGCACATCAGCGCCACAGATGCCTTTGCCATCTCTTGGATGCCCTGATAAAGC |
| | | CAACAACTGTGAGTACTATTCATTGCCCAGGAAAACAGGAGGGAAGAGATTCAGTGACATGGGGCAGTGACAAAACAA |
| | | ATAAAGTGGCTCGGGAAATGGCTATACAGGAGCCTATCCTGGTTACAGGCCTGCAAGAGACAGCCACTGAGAACTAG |
| | | GATTAAACTAAGGGGATGGCCTCACTTAGAAAAGGCCCAAGTTGTTTTAAAAGATAAAAAGACNATGACACACTTGAGG |
| | | GGAAGGCTATACTCCCCAGAAAACAAAGAAAAAGACTCACTTTGCCAAATACAGAAATGGACTCATTTAGGAGATAAAA |
| | | ANTTTGTCCCAAGTAGTTAAGGTATATGTAATANACTTTAAAAATTTTANCCCAAGAGAGACAGTAAAAA |
| Enolase alpha | X02610 | TAGNTGNCATAATAAATACTCAAGTATGCATCAAAGTTTGGTACCGAGCTCGGATCCACTAGTACCGGCCCGCCAGTGT |
| | | GCTGGAATTCGCCCTTATCGCGGGATCCGCTGCAGGCGAAAGTCCTGCAACTGCCTCCTGCTCAAAGTGAACCAGATT |
| | | GGCTCCGTGACCGAGTCTGTGCAGGCGTGTAAGCTGGCCCAGTCCAATGGCTGGGGTGTCATGGTGTCCCATCGATC |
| | | TGGGGAGACTGAGGACACTTTCATTGCCGACCTGGTGGTGGGGCTCTGCACTGGGCAGATCAAGACTGGTGCCCCCT |
| | | GCCGATCTGAGCGCCTGGCCAAGTACAATCAGATCCTTAGAATCGAGGAGGAGCTGGGCAGCAAAGCCAAGTTTGCC |
| | | GGCAGGTCCTTCAGGAACCCCCTGGCCAAGTAAGGCATGGACCGGAGATCCCTGGAGCTACCAGATCCTCTGTCTCT |
| | | GTCATCCAGGCGGCTCAAGGCTGGCCCAGTGCTTGCCCCTCCCATGTCACTGCTTCCTTAGATGTCCACCCCGACCAC |
| | | CTGGAGCCCTGCTGGAGCCCCCAGCTTTGTAAAAGCTTGGCCAAAAGGGCGAATTCTGCAGATATCCATCACACTGGC |
| | | GGCCGCTCGAGCATGCATCTAGAGGGCCCAATTCGC |
| Epidermal growth factor | U04842 | ATGCCATGATTACGCCAAGCTATTTAGGTGNCACTATAGAATACTCAAGCTATGCATCAAGCTTGGTACCGAGCTCGGA |
| | | TCCACTAGTAACGGCCGCCAGTGTGCTGGAATTCGCCCTTAAGGGCCTCAGACCTGCCCCAGCAAACAGAGCCAGTT |
| | | CCGTAGAAACTGGGAGCAGACAGAAGGTACCGAAGGTGAAATAGCAAACCAGGCTGAAGGGTGGTAGAGCGGCAGA |
| | | TCTGGTACTCCTGTCTCCACGGCTAATCACTGCTCAGGGTCCTGAAGATAACTGCATAGCTGCATAGCTGCATAGCCTC |
| | | GACTTCTGCTTCTTGCTTCAAGCAGTCCCGTTGAAGACGATCAAAAGAGAAGTGGAGAAAAATCATCAGAAACCGAAGT |
| | | CAAGACGTTCACGTGTTGTAAGCTGTGTCTTCTTCCCTGGACTGTTGGGCTCTTTTCCTTGTTGTCTCAGAAGAAATGG |
| | | GTTAAAGCAGGCGATCATATGCTTTGTTGATTGCACAGTAGATGATATGATCTACATAGATCTTAGCTCACTCTCACGGA |
| | | AAGGCTGGAACATTATAGATGCTGCTAAGATACACTGCAAGTGTGGCCCCTGCTCATAAAGCTTAAGGGCGAATTCTG |
| | | CAGATATCCATCACACTGGCGGCCGCTCGAGCATGCATCTAGAGGGCCCAATTCCCAA |
| Equilbrative nitrobenzylthioinosine- | AF015304 | TTATGACATGATTACGAATTTAATACGACTCACTATAGGGAATTTGGCCCTCGAGGCCAAGAATTCGGCACGAGGGGCA |
| sensitive nucleoside transporter | | GCACTGTGTCCCTAGCAATCAGCACCCTCCAAAGGTATCCAGGCCTAGAGGTTTTATCCGAGTCCCTAGGCCCAAGTG |
| | | CGCCATGAGGACGGACACCCGCTGAAGCTTTGCTGGTCATTGCAGCCTGGTCAGGACAGCCGCTGGTTGCCGGTCCT |
| | | GGTCGCCTGCAGGGTTGTGTTTATCCCCCTGCTGATGCTCTGCAATGTGAAGCAGCACCACTACCTGCCCTCCCTCTT |
| | | TAAGCATGATGTCTGGTTCATCACCTTCATGGCCGCCTTTGCCTTCTCCAATGGCTACCTCGCCAGCCTCTGCATGTGC |
| | | TTCGGGCCCAAGAAAGTCAAACCGGCTGAGGCAGAGACTGCCGGAAACATCATGTCCTTCTTTCTGTGTCTGGGCCTG |
| | | GCTCTGGGAGCTGTGTTGTCCTTCTTGTTAAGGGCACTTGTGTGAGCGACCCTGTGTGGACAGAGGAACTACACTGCC |
| | | TGCTTCCTGCTCACTTCCTTCGCTGTTAGGGACGAGCAGGGGTCCAGAGGGCTGCTCTTCTGCTTTCCTCCGGGCTGG |
| | | GCCCAGATGTCCAGGAACAAAGGANGGACCTCTGAGGATGGACTTGGGATTGGGGGTCANANTGGTANGGGGACAAT |
| | | GGTCTCTGA |
| Extracellular-signal-regulated kinase 1 | M61177 | TGGGAATTGGCCCTCTAGATGCATGCTCGAGCGGCCGCCAGTGTGATGGATATCTGCAGAATTCGCCCTTCGCGGGA |
| | | TCCAGCACGACCACACTGGCTTTCTGACCGAGTATGTGGCCACACGCTGGTACCGAGCCCCAGAGATCATGCTTAACT |
| | | CCAAGGGCTACACCAAATCCATTGACACCTGGTCTGTGGGCTGCATTCTGGCTGAGATGCTCTCCAACCGGCCTATCT |
| | | TCCCCGGCAAGCACTACCTGGACCAGCTCAACCACATTCTAGGTATACTGGGTTCCCCATCCCAAGAGGACCTAAATT |
| | | GTATCATTAACATGAAGGCCCGAAACTACCTACAGTCTCTGCCCTCTAAAACCAAGGTGGCTTGGGCCAAGCTTTTTCC |
| | | CAAATCTGACTCCAAAGCTCTTGACCTGCTGGACCGGATGTTAACCTTTAACCCAAACAAGCGCATCACAGTAGAGGAA |
| | | GCACTGGCTCACCCTTACCTGGAACAGTACTATGATCCGACAGATGAACCAGTGGCTGAGGAGCCATTCACCTTTGAC |
| | | ATGGAGCTGGATGATCTCCCCAACTGCAGGGCCAAAGGGCGAATTNCAGCACACTGGCGGNCGNTACTAGTGGATCG |
| | | AGCTCGGTACCAAGCTT |
| Fas antigen | D26112 | GGNCCAGCTTGGTACCGAGCTCGGATCCACTAGTAACGGCCGCCAGTGTGCTGGAATTCGCCCTTCGCGGGATCCCT |
| | | GCACCTCGTGTGGACTTGAAGACATCCTTGAGCCTTGCACACGAACCAGCAACACCAAATGCAAGAAACAAAGTTCCA |
| | | ATTATAAGCTCCTTTGGCTGCTGATCCTCCCCGGTTTGGCAATTCTATTTGTGTTCATATATAAAAGGTACCGGAAGAGG |
| | | CAACCTGGTGACCCTGAATCTGGAATCCCAAGTCCTGAAAGTGTGCCAATGAATGTCTCAGATGTTAACTTGAATAAAT |
| | | ACATCTGGAGAACTGCCGAAAAAATGAAAATCTGTGACGCTAAAAAATTTGCTCGGCAGCACAAGATCCCGGAAAGCA |
| | | AGATCGATGAGATCGAGCACAACAGCCCCCAAGATGCAGCTGAGCAGAAAATCCAGCTGCTCCAGTGCTGGTATCAAT |
| | | CTCACGGGAAGACTGGTGCGTGTCAAGCTTTAATCCAGGGTCTCAGAAAAGCTAACCGCTGCGACATCGCAGAGGAAA |
| | | TTCAAGCCATGGTCTGGGACTGCAGGGCCAAGGGCGAATTCGATATCGCGGGCCGCTCGAGCATGCATNTAAGAGGG |
| | | CCCTATTCTATAGNGTCACTAAATGCTAAAACTCGCTTGATCAACCTCACTGT |
| Fatty acid synthase | M76767 | AATACTCAAGTTATGCATCAAGCTTGGTNCCGAGCTCGGATCCACTAGTACCGGCCGCCAGTGTGCTGGAATTCGCCC |
| | | TTCGCGGGATCCACCTTTAAGTCACCCTGAAGGCATCACCATAGCTACAGCCTCACCCAGGGGTAGAGAATAGTACTG |
| | | TCTACTTGTTGACTACCTGGCAGTTGGTGCCAGCCCCTATAGAGGAAAACAGCAGTGTGTGGCCTCTGTGAGAAGCAT |
| | | ATCCCTGGAAACAGGTGACCAGAGCAGAGGGCTAATGCCTACCTGAGTCACACAAAACTGACCAGGCTTGAGTGTCCA |
| | | GAAGAGTCTATCAGAAGGCCACAGCATTCAGTCCTATCCACAGAGAGCAGCAGACTAAGTTGTCTCCTCGCCAGCTTA |
| | | GAAAACTGCAGTGCTGGGGTACAGGTAGGGTGTTCAGGAGGTCCGGGCCCCAGTGATTAGTCTAAGACTGAAGCATC |
| | | TGGTTGGCTGTGGTCCCACCTAGAAAATTCTTAAAGCTCTTGTCATGTACTTCCTGGGAAGGACCTACCCTGTCTCAAT |
| | | AATGTCTCTAGCTCGTTGGAGTCTACTGACTCAAAAGCTTGGCCAAGGGCGAATTCTGCAGATATCCATCACACTGGCG |
| | | GCCGCTCGAGCATGCATCTAGAGGGCCCNATTCCC |
| Ferritin H-chain | U58829 | GGATTGGCCNCCTAGAGCATGCTCGAGCGGCCGCCAGTGTGATGGATATCTGCAGAATTCGCCCTTTTGGCCAAGCTT |
| | | TCACATGGCCTTGGGGACGTCAGCTTAGCTCTCATCACCGTGTCCCAGGGTGTGCTTGTCAAAGAGATATTCTGCCAT |
| | | GCCAGATTCAGGGGCTCCCATCTTGCGTAAGTTGGTCACGTGGTCACCCAGTTCTTTAATGGATTTCACCTGCTCATTC |
| | | AGGTAATGCGTCTCAATGAAGTCACATAAGTAGGGATCATTCTTGTCAGTAGCCAGTTTGTGAAGTTCCAGTAGTGATT |
| | | GATTCACACTCTTTTCCAAGTGCAGTGCACACTCCATTGCATTCAGCCCGCTCTCCCAGTCATCACGGTCAGGTTTCTT |
| | | TATATTCTGCAGGAAGATTCGTCCACCTCGCTGGTTCTGCAGCTTCATCAGTTTCTCAGCATGCTCCCTCTCTTCATGAG |
| | | ATTGATGGAGAAAGTATTTGGCAAAGTTCTTCAGGGCCACATCATCCCGGTCAAAATAACAAGACATGGACAGATAGAC |
| | | GTAGGAGGCATACAACTCCAGGTTGATCTGGCGGTTGATGGCAGGATCCCGCGATAAGGGCGAATTCCAGCACACTG |
| | | GCGGCCGTTACTAGTGGATCCGAGCTCGGTACCAAGCTTGAGCATAGCTTGAGTATTCTATAGTGTCACCTAAATAGCT |
| | | TGGCGTAATCATGGNCATAGCN |
| Focal adhesion kinase (pp125FAK) | AF020777 | TTGCGAATTGGGCCCTCTAGATGCATGCTCGAGCGGCCGCCAGTGTGATGGATATCTGCAGAATTCGCCCTTATCGCG |
| | | TCTAGATGCCCTTTGGGAGATCTTCTCTAGCATTCCACCAGCAGCGAGGAAGTAACCTTGTCCTCGGTCGCCCGCACT |
| | | CACAGCTCCAACTGATGACAGCTAGCTGAAAGTCTCTCTTGTATAAGTTTAACTACTTATACATGGTTTTGATTCTGTTAT |
| | | TTTTCTAAATAGTGTTCAGAAAACTCTGGATCCAAAATGTGGCATTTTTCTGAGAATGAAGATTGTACATTAAGAAGCTTT |
| | | TAAAATATACTTGGTGTTCAAATTGGGATGTATGTTCCTAGAAGTGGTAATTCATACTGACCATGACTTCGAATTGGAAG |
| | | AGAAGTGTCTGAGGAGGGAGGGCTCCCAAGACACTGAGACTGGCTATCCTTCCTGCCAGAATTCCTGTCCAGACTGAA |
| | | TTGCAATATGCTAATCTCATTTATAGAGAAAGTGCATAAAAGCTATATTTTGAAGAATGAGTGGTTTCAAAAGAAAACTTC |
| | | TGCCCTCCCTGTCAAGGGCGAATTCCAGCACACTGGCGGCCGTTACTAGTGGATCCGAGCTCGGGACCAACTTGATG |
| | | CATAACTTGGAGTATTCTATAAGTGNCCCTAAAATAGCTTGGCGTAATCN |
| Gadd153 | U36994 | GGGNTCCACTAGTANCGGCCNGCCAGTGTGCTGGAATTCGCCCTTATCGCGGATCCNTGAGTCTCTGCCTTTCGCCTT |
| | | TGAGACAGTGTCCAGCTGGNAGCTGGAAGCNTGGTATGAGGATCTGCAGGAGGTCCTGTCCTCAGATGAAATTGGGG |
| | | GCACCTATATCTCATCCCCAGGAAACGAAGAGGAAGAATCAAAAACCTTCACTACTCTTGACCCTGCATCCCTAGCTTG |
| | | GCTGACTGAGGAGCCAGGGCCAGCAGAGGTCACAAGCACCTCCCAAAGCCCTCGCTCTCCAGATTCCAGTCAGAGTT |
| | | CTATGGCTCAGGAGGAAGAAGAGGAAGATCAAGGAAGAACTAGGAAACGGAAACAGAGTGGTCAGTGCGCAGCCCG |
| | | GGCTGGGAAACAGCGCATGAAGGAGAAGGAGCAGGAGAATGAGAGGAAAGTGGCACAGCTTGCTGAAGAGAACGAG |
| | | CGGCTCAAGCAGGAAATCGAGCGCCTGACCAGGGAGGTAGAGACCACACGGCGGGCTCTGATCGACCGCATGGTCA |
| | | GTCTGCACCAAGCATGAACTGTTGGCATCACCTCCTGTCTGTCTCTCCCGGAGTGTACCCAGCACCATCACGCCAGTG |
| | | CCAAGCATGTAATCTCCAGTGCACATGCTGAGGAGGAAGCTTGGCCAAAAGGGCGAATTCTGCAGATATCCATCACAC |
| | | TGGCGGCCGCTCGAGCATGCTTTAGGGGNCCAATC |
| Gadd45 | L32591 | TTNGGAATTGGGCCCTCTAGATGCATGCTCGAGCGGCCGCCAGTGTGATGGATATCTGCAGAATTCGCCCTTCGCGTC |
| | | TAGAGACTTTGGAGGAATTCTCGGCCGCAGAGCAGAAGATCGAAAGGATGGACACGGTGGGCGATGCCCTGGAGGAA |
| | | GTGCTCAGCAAGGCTCGGAGTCAGCGCACCATAACTGTCGGCGTGTACGAGGCAGCCAAGCTGCTCAACGTAGACCC |
| | | GGACAACGTGGTCCTGTGCCTGCTGGCTGCGGATGAAGATGACGACCGGGACGTGGCTCTGCAGATCCATTTCACCC |
| | | TCATTCGTGCTTTCTGTTGCGAGAACGACATCAACATCCTGCGGGTCAGCAACCCGGGTCGGCTGGCAGAGCTGTTGC |
| | | TACTGGAGAACGACAAGAGCCCCGCTGAGAGCGGGGGCGCTGCGCAGACCCCGGACTTACACTGTGTGCTGGTGAC |
| | | GAACCCACATTCATCACAATGGAAGGATCCTGCCTTAAGTCAACTTATTTGTTTTTGCCGGGAAAGTCGCTACATGGAT |
| | | CAGTGGGTGCCAGTGATTAATCTCCCCGAACGGTGATTCAAGCTTGGCCAAGGCGAATTNCAACACACTGCGGGCGG |
| | | TACTATGGATCCAACTCGGACCAACTTGATGCATACTT |
| Gamma-actin, cytoplasmic | X52815 | CTTTATGACATGATTACGAATTTAATACGACTCACTATAGGGAATTTGGCCCTCGAGGCCAAGAATTCGGCACGAGGGA |
| | | TTTGCCCTGGCAAATGTACACACCTCATGCTAGCCTCATGAAACTGGAATAAGCCTTTGAAAAGAAATTTGTCCTTGAAG |
| | | CTTGTATCTGATATCAGCACTGGATCGTAGAACTTGTTGCTGATTTTTGACCTTGTATTCAAGTTAACTGTTCCCTTGGTA |
| | | TTAACCAACAGCAGACTTCCAGGATTTCCCGAGGCTGGCAAGGGTTCCTGAACTAGTTACCACTTCTTTTCTTGCCAGT |
| | | CTAACAGGGTGGGAAAGTCCGAGCCTTAGGACCCAGTTTCTGTTCTGGTTTTTTCCCTCCTGACCTCCATGGGTTGTTA |
| | | CTTGCCTTGAGTTGGGAACGTTTGCATCGACACCTGTAAATGTATTCATCCTTTTAATTTATGTAAGGTTTTTGTACTCAA |
| | | TTCTTTAAGAAATGACAAATTTTGGTTTTCTACTGTTCAGTGAGAACATTAGGCCCCAGCAACACGTCATTGTGTAAAGA |
| | | GAAATAAAAGTGCTGCAGTAACNNCNTAAAAANNCCANCNNNAACNNANAAACCNATTGCGGCCGCAAGCTTATTCCC |
| | | TTTAGNGANGGGTTAATTTTAGCTTTGGCNCTGGCCGCCGTTTTTACAACGTCNGNGACTGGNNAAAN |
| Gap junction membrane channel protein | NM_017251 | TATGACATGATTACGAATTTAATACGACTCACTATAGGGAATTTGGCCCTCGAGGCAAGAATTCGGCACGAGGAGCAGT |
| beta 1 (Gjb1) | | TGCAACCAGGTGTGGCAGTGCCAGGGAGGTGTGAATGAGGCAGGATGAACTGGACAGGTCTATACACCTTGCTCAGT |
| | | GGCGTGAATCGGCATTCTACAGCCATTGGCCGAGTATGGCTGTCCGTCATCTTTATCTTCAGAATCATGGTGCTGGTG |
| | | GTGGCTGCAGAGAGCGTGTGGGGTGATGAGAAGTCTTCTTTCATCTGTAACACCCTCCAGCCGGGCTGTAACAGCGT |
| | | CTGCTATGACCATTTTTTCCCCATCTCCCATGTGCGCCTGTGGTCCCTGCAACTCATCTTGGTTTCCACCCCAGCTCTC |
| | | CTCGTGGCAATGCACGTGGCTCACCAACAACACATAGAAAAGAAAATGCTACGGCTTGAGGGGCACGGGGACCCCCT |
| | | TCACCTGGAGAGGTAAAGAGGCACAAGGTGCACATCTCAGGGACACTGTGGTGGACCTATGTCATCAGTGTGGTGTTC |
| | | CGGCTGCTGTTTGAGGCTGTCTTCATGTATGTCTTCTATCTGCTCTACCCGGGCTATGCCATGGTGCGGCTGGTCAAGT |
| | | GTGAGGCCTTCCCCTGCCCCAACACGGTGGACTGCTTCGTGTCCCGCCCCACTGAGAAAACCGTCTTCACTGTCTTTA |
| | | TGCTCGCCGCCTC |
| Glucokinase | J04218 | TTGCGAATTGGGCCCTCTAGATGCATGCTCGAGCGGCCGGCCAGTGTGATGGATATCTGCAGAATTCGCCCTTTCACGA |
| | | TTCTTGCTTCCCAAGCGTGGGTCTGGCCTCCCAAGGGAATGCTTCCTGGACCTTGCAATGGCCTGGCTTCCCTGGGG |
| | | GGGACACACCTTCATGGGGAGGTAACTTCAGCAGTTCGGCCAGACCAGACCCCAGGAGAGTAAGGGCTGCTAGTCAC |
| | | CCAGACCTGGCTGTTTTCTTGTCTGTGGCTGAAGAGGCCGGGGAGCCATGAGAGACTGACTATCCGGCTACATGGAG |
| | | AGGACTTTCCAGGCATGAACATGCCAGAGACTGTTGCCTTCATATACCTCCACCCGAGTGGCTTACAGTTCTGGGATG |
| | | AACCCTCCCAGGAGATGCCAGAGGTTAGAGCCCCAGAGTCCTTGCTCTAAGGGGACCAGAAAGGGGAGGCCTCACTC |
| | | TGCACTATTCAAGCAGGAATCATCTCCAACACTCAGGTCCCTGACCCANGAGGAAGAAGCCACCCTCAGTGTCCCTCC |
| | | AGAGACCACCCAGGTAAGGGCGAATTCCAGCACACTGGCGGCCGGTACTAGTNGATCCGAGCTCGGACCAA |
| Glucose-regulated protein 78 | M14050 | GAATNGGCCCTCTAGATGCATGCTCGAGCGGCCGCCAGTGTGATGGATATCTGCAGAATTCGCCCTTNGCGGGANCC |
| | | CTGACTGGAATCCCTCCTGCTNCCCGTGGGGTACCCCANATTGNANNCACCTTNGANATACATGTGAATGGNATTCNT |
| | | NGAGTGGCANNTGAAGACANAGGGACAGGNAACANNNACNNANTCACCATCACCAATGACCAAAACCGCCTGACCCC |
| | | TGAANAAATTGAAAGGATGGTCAGTGACGCCGANAANTNNGCTGAGGANGACNAAANGCTCANAGAGCGCATNGACA |
| | | CGANGAATGAATTGGAAAGCTATGCTTACTCTCTTAAGAACCANATCCGGANATNAAGAGANGCTGGGAGGTAAGCTG |
| | | NCTNCTGAANATAANNAGACCATGGAGAAAGCTGTAGAGGAAAAGATCGAATGGCTGGAAAGCCACCAGGATGCAGA |
| | | CATTGAACACTNTAAAGCTNNNAAGATNGAACTAGACNANATTGTTCANCCAATTATGAGCAAACTCTATNGAAGTGGA |
| | | CGCCCTCCCCCAACTGGGGANGAAGAAGCTTGGCCCAGGGCGAATTCCATCACACTGGCNGGCGGTACTATTGGATC |
| | | GATCTCGNTCCAACCTGANGCATAGCTTGAGTNTTCTATNNTG |
| Glutathione S-transferase theta-1 | X67654 | NGCNCTATAGAATACTCAAGCTATGCATCAAGCTTGGTACCGAGCTCGGATCCACTAGTAACGGCCGCCAGTGTGCTG |
| | | GAATTCGCCCTTGTGTGGATGAGTACCTGGCATGGCAGCATACGACCCTTCGGAGAAGCTGTCTCCGGACCCTGTGG |
| | | CATAAGGTGATGTTCCCCGTTTTCCTGGGTGAGCAGATACGCCCGGAGATGCTGGCAGCCACATTGGCAGATCTGGAT |
| | | GTTAACGTACAGGTGCTGGAAGACCAGTTCCTCCAGGACAAAGACTTCCTTGTCGGACCGCACATCTCCCTGGCTGAC |
| | | GTGGTAGCTATCACAGAGCTGATGCATCCTGTAGGTGGTGGCTGCCCAGTCTTTGAAGGGCGTCCCAGGCTGGCTGC |
| | | ATGGTACAGGAGAGTGGAGGCAGCTGTGGGGAAGGACCTCTTCCTGGAGGCCCATGAGGTCATCCTGAAGGTGAGA |
| | | GACTGTCCACCTGCTGACCCCGTCATAAAGCAAAAGCTGATGCCCAGAGTGCTGACCATGATCCAGTGACGTCAGAAG |
| | | CTTCATCCCTGCACCAGCCAGGCCACTCGCATTACACGACTGTAAGGGCGAATTCTGCAGATATCCATCACACTGGCG |
| | | GCCGCTCGAGCATGCATCTAGAGGGCCCAA |
| Glycine methyltransferase | NM_017084 | TATGACATGATTACGAATTTAATACGACTCACTATAGGGAATTTGGCCCTCGAGGCCAAGAATTCGGCACGAGGCAGC |
| | | CTTTGACAAGTGGGTCATTGAAGAAGCCAACTGGTTGACTCTGGACAAAGATGTGCCAGCAGGAGATGGCTTTGACGC |
| | | TGTCATCTGCCTTGGGAACAGTTTTGCTCACCTGCCGGACAGCAAAGGTGACCAGAGTGAGCACCGGCTGGCGCTAA |
| | | AGAACATCGCAAGCATGGTGCGGCCCGGGGGCCTGCTGGTCATCGACCACCGCAACTACGACTACATCCTCAGCACG |
| | | GGCTGTGCACCCCCAGGGAAGAACATCTACTATAAGAGTGACCTGACCAAGGACATTACGACGTCAGTGCTGACAGTA |
| | | AACAACAAAGCTCACATGGTAACCCTGGACTACACAGTGCAGGTGCCAGGTGCTGGCAGAGATGGCGCTCCTGGCTT |
| | | CAGTAAGTTTCGGCTCTCTTACTACCCACACTGTTTGGCGTCTTTCACGGAGTTGGTCCAAGAAGCCTTTGGGGGCAG |
| | | GTGCCAGCACAGCGTCCTGGGTGACTTCAAGCCTTACAGGCCCGGCCAGGCCTACGTTCCCTGCTACTTCATCCACGT |
| | | GCTCAAGAANACAGGCTGANCCTGGCTNCNGCTTCCACCCTAANAACATCCCTACCACAGATATTGCAGANAT |
| Heme oxygenase | NM_012580 | TTNTGACATGATTCGAATNNAANACCGACTCACTATAGGGAATTTGGCCCTCGAGGCCAAGAATTCGGCACGAGGACC |
| | | CGCTACCTGGGTGACCTCTCAGGGGGTCAGGTCCTGAAGAAGATTGCGCAGAAGGCCATGGCCTTGCCAAGCTCTGG |
| | | GGAAGGCCTGGCTTTTTTCACCTTCCCGAGCATCGACAACCCCACCAAGTTCAAACAGCTCTATCGTGCTCGCATGAA |
| | | CACTCTGGAGATGACCCCCGAGGTCAAGCACAGGGTGACAGAAGAGGCTAAGACCGCCTTCCTGCTCAACATTGAGC |
| | | TGTTTGAGGAGCTGCAGGCACTGCTGACAGAGGAACACAAAGACCAGAGTCCCTCTGCAGAGACGCCCCGAGGAAAA |
| | | TCCCAGATCAGCACTAGTTCATCCCAGACACCGCTCCTGCGATGGGTCCTCACACTCAGTTTCCTGTTGGCGACCGTG |
| | | GCAGTGGGAATTTATGCCATGTAAATGCAGTGTTGGCCCCCAGAGGCTGTGAACTCTGTCTCATGTAGCCTTCTCTCTG |
| | | CAGGGGAGAATCTTGCCTGGCTCTCTTTTCTTGGGCCTCTAAGAAAGCTTTTGGGGTTCCTCGCCCCCTTCCTGTGTCN |
| | | TTCCTTTNCTCTCTTGGAATGGGAAGGAGATGCCTGGCACATTTCT |
| Hepatic lipase | M16235 | TTGGGAATTGGCCCTCTAGATGCATGCTCGAGCGGNCCGCCAGTGTGATGGATATCTGCAGAATTCGCCCTTCGCGG |
| | | GATCCCGTTGCAACAGTCTGGGCTATGACATCCGCAGGGATCGGCCACGCAAGAGCAAGACACTCTTCCTCATCACCC |
| | | AAGCCCAGTCCCCCTTCAAAGTTTATCATTACCAGTTCAAGATCCAGTTCATCAATCAAATGGAGAAGCCAATAGAGCC |
| | | TACTTTTACCATGACACTGCTGGGGACAAAAGAAGAAATAAAGAAAATTCCCATCACCCTGGGCGAAGGAATTACCAGC |
| | | AATAAAACCTATTCCTTACTTATCACACTGGACAAAGACATCGGCGAGTTGATCATGCTCAAGTTCAAGTGGGAAAACA |
| | | GCGCAGTGTGGGCCAATGTCTGGAACACAGTGCAGACCATAATGCTATGGGACACAGAGCCTCACTACGCGGGCCTC |
| | | ATTCTGAAGACCATCTGGGTCAAAGCTGGAGAGACGCAGCAAAGAATGACATTTTGCCCTGATAATGTGGATGATCTCC |
| | | AGCTTCACCCCACCCAGGAGAAAGTCTAAGCTTGGCCAAGGGCGAATTCCAAGCACACTGGCGGCCGTTACTAGTGG |
| | | NTCCGAGCTCGGTCCAAGCTTGATGCATAGCTTGGAGTATTCTATAGTGNCACCCTAAA |
| High affinity IgE receptor gamma chain | AA957422 | TATGACATGATTACGAATTTAATACGACTCACTATAGGGAATTTGGCCCTCGAGGCCAAGAATTCGGCACGAGGCGCG |
| (FcERIgamma) | | TGATCACCAGCCCCCAGCGCTGCAGCCCCCGCCCAGGATGATCCCAGCGGTGATCTTGTTCTTGCTCCTTTTGGTGGA |
| | | AGAAGCAGCTGCCCTAGGAGAGCCGCAGCTCTGCTATATCCTGGATGCCATCCTGTTTTTGTATGGTATTGTCCTTACC |
| | | CTGCTCTACTGTCGACTCAAGATCCAGGTCCGAAAGGCAGACATAGCCAGCCGTGAGAAATCAGATGCTGTCTACACG |
| | | GGCCTGAACACCCGGAACCAGGAGACATATGAGACTCTGAAACATGAGAAACCACCCCAATAGCTTTACAACACGTGT |
| | | TCTCAGCTGCATTCCTTTTCCGCTTTTAATTCTCTCCTCGCCCTCATGATTGACGTGGCTGTGCTACCTCCGTGCTTCTG |
| | | GAACTAGCTGACCTTATTCCCAGAACCATGCTAGGCTCTAAATCAATGTCCCCATATCCACCAAAGACTTACTCACTGA |
| | | CATTTCTCTTCTCCCATCCTCCTTTGCTTCATTCCTCTTTCCTTCCCTGATCCTCTGTGCTCACTAAACAATGGGAAGGG |
| | | ATTACCCCCCAATAAAGCTGCCAGAGATCACGCTCAAAAAAAAAAAAAAAAAAAAAAACATTGC |
| H-rev107 | NM_017060 | TCAGGTTGGGAAACTGTTTGGAAGGGCGATCGGTGCGGCCNTNTTCGCTTATTACGCCAGNTGGGCGAAAGGGGGAA |
| | | TGTGCTGCAAGGCGATTAAGTTGGGTAACGCCAGGGTTTTCCCAGTCACGACGTTGTAAAACGACGGCCAGTGCCAAG |
| | | CTAAAATTAACCCTCACTAAAGGGAATAAGCTTGCGGCCGCGTGATGTGGCTGGGAAGGACAAGTACCAGGTCAACAA |
| | | CAAGCACGACAAGGAGTACACTCCGCTGCCTCTGAACAAGATCATCCAGCGAGCTGAGGAGCTGGTGGGGCAGGAG |
| | | GTGCTGTACAGGCTGACCAGTGAGAACTGTGAGCACTTCGTGAACGAACTGCGTTATGGAGTCCCTCGGAGTGACCA |
| | | GGTCAGAGATGCCGTCAAGGTGGCGACCGTCACTGGAGTGGGCTTGGCGGCCTTGGGCCTCATTGGAGTCATGCTCT |
| | | CAAGAAACAAGAAACAGAAGCAGTGAGCTGAATGACTATCCAGCTTTAGGGCTCTTCTTTTGCTAGAGGGTTGGAGTTT |
| | | GATTTATAGATTCTACTGCTTTATAATTAGGTATATTTTCACAATATACAATAAACCACAAGAAGGGAATTTTCATGGAAA |
| | | AAAAAAACCTCGTGCCGAATTCTTGGCCTCGAGGGCCAAATTCCCTATAGTGAGTCGTATTAAATTCGTAATCATGTCAT |
| | | AGANNNNNNCNN |
| Hypoxanthine-guanine phosphoribosyl- | X62085 | GAATTGGGCCCTCTAGATGCATGCTCGAGCGGCCGCCAGTGTGATGGATATCTGCAGAATTCGCCCTTCGCGGGATC |
| transferase | | CTGCTGAAGATTTGGAAAAGGTGTTTATTCCTCATGGACTGATTATGGACAGGACTGAAAGACTTGCTCGAGATGTCAT |
| | | GAAGGAGATGGGAGGCCATCACATTGTGGCCCTCTGTGTGCTGAAGGGGGGCTATAAGTTCTTTGCTGACCTGCTGG |
| | | ATTACATTAAAGCGCTGAATAGAAATAGTGATAGGTCCATTCCTATGACTGTAGATTTTATCAGACTGAAGAGCTACTGT |
| | | AATGACCAGTCAACGGGGGACATAAAAGTTATTGGTGGAGATGATCTCTCAACTTTAACTGGAAAGAACGTCTTGATCG |
| | | TTGAAGATATAATTGACACTGGTAAAACAATGCAGATTTTGCTTTCCTTGGTCAAGCAGTACAGCCCCAAAATGGTTAAG |
| | | GTTGCAAGCTTGCTGGTGAAAAGGACCTCTCGAAAGTGTTGGATACAGGCCAGACTTTGTTGGATTTGAAATTCCAGAC |
| | | AAGTTTGTTGTTGGATATGCCCTTGACGCAGGCCAAGGGCGAATTCCAGACACTGGCGGNCGTACTAGTGGATCCGA |
| | | GCTCGGACCAAGCTTGATGCATACTT |
| ID-1 | D10862 | GGATTTCGCCCTTATCGNGGGATCCAAAGCGTTGCCATCTCGCGCTTGCGCTTGGNACGNCGCCTGCCCCCCCTTGC |
| | | TGGGACGAACAGGCAGGTGAACGTTCTGCTCTACGACATGAACGGCTGCTACTCACGCCTCAAGGAGCTGGTGCCTA |
| | | CCCTGCCTCAGAACCGCAAAGTGAGCAAGGTGGAGATACTGCAGCATGTTATCGACTACATCAGGGACCTGCAGCTG |
| | | GAGCTGAACTCTGAGTCTGAAGTCGCGACCGCCGGAGGCCGGGGGCTGCCCGTCCGGGCCCCGCTCAGCACCCTGA |
| | | ACGGCGAGATCAGTGCCTTGGCGGCCGAGGCGGCATGTGTTCCAGCCGACGACCGCATCTTGTGTCGCTGAGGCGG |
| | | CGCACTGAGGAACCAGATGGACTCCAGCCCTTCAGGAGGCAAGAGGAAAAAAAGTGCTCTCGGTTCCCCAGAGCAAC |
| | | CCGGGGAAAGACACTACCGCGGCCACGGGACTCTTGACGGATCTGTCCAGGGGGTAGAGGGTTGATCAACGGAGTC |
| | | TCGCCCTCTCCAAGCTTGGCCAAAGGGCGAATTCTGCAGATATCCATCACACTGGCGGCCGCTCGAGCATGCATCTAG |
| | | AGGGCCNGTTTTCCAA |
| IgE binding protein | NM_031832 | TATGACATGATTACGAATTTAATACGACTCACTATAGGGAATTTGGCCCTCGAGGCCAAGAATTCGGCACGAGGGGAG |
| | | CACTAACCAGGAAAATGGCAGACGGCTTCTCGCTTAATGATGCCTTAGCTGGCTCCGGAAACCCAAACCCTCAAGGAT |
| | | GGCCTGGTGCATGGGGGAACCAGCCTGGGGCAGGAGGCTACCCAGGGGCCTCCTATCCTGGGGCCTACCCAGGACA |
| | | GGCTCCTCCAGGGGGTTATCCTGGACAGGCTCCTCCTAGTGCCTATCCGGGCCCAACTGGCCCTAGTGCTTATCCTG |
| | | GCCCAACTGCCCCTGGAGCTTATCCTGGCCCAACTGCCCCCGGAGCCTTCCCAGGGCAACCTGGGGGACCTGGAGC |
| | | CTACCCCAGTGCTCCTGGGGCCTATCCTGCTACTGGCCCCTATGGTGCCCCGACTGGACCACTGACAGTGCCCTACG |
| | | ATATGCCCTTGCCTGGAGGAGTGATGCCTCGCATGCTGATCACAATCATGGGCACAGTGAAGCCCAACGCAAACAGTA |
| | | TCACTCTGAATTTCAAGAGAGGGAACGACATCGCCTTCCACTTTAACCCCCGCTTCAATGAGAACAACAGAAGAGTCAT |
| | | CCGTGTGCAACACGAAGCAGGACAATTAACTGGGGAANGGAAAAAAAGACAG |
| 1kB-a | X63594 | TTGCGAATTGGGCCCTCTAGATGCATGCTCGAGCGGCCGCCAGTGTGATGGATATCTGCAGAATTCGCCCTTATCGCG |
| | | GGATCCTGCCCGAGAGTGAGGATGAGGAGAGCTATGACACGGAGTCAGAATTCACGGAAGATGAGTTGCCCTACGAT |
| | | GACTGTGTGTTTGGAGGCCAGCGTCTGACGTTATAAGAGGAAAGTGGCCAAAGGAACGTGGACTTGTATATTTGTACA |
| | | AATAGAGTTTTCTAAAAAAAAAAAAGAAAAAGAAAAAAAAATAAGAAAGGGTATACTTAGCACCACAGCACACA |
| | | GCGCCTAGCCCCGAGCATTCTATTGTGGTGATTCAGCTGTTGTTCTTGTGAAATTTGGGGAAACAGATCACTGAAATTC |
| | | AAAGGAAATGTCTCCTAAGGTTCATGTTTGTGGGATTTTTTTTTTTGAGGTGATTTTCCAGAATCTGATAAAAGGACCAC |
| | | ATTTTATTTATTGTGCTTTTGGTTGAACCGCCATAGACTGTAGCTGACCCCAGTGTGCCCTCTCACGTAAGAACCAGGT |
| | | GTTCAGTGGTATGTGCTTAAGCACCCCAGCTGAGAAGCTTAAGGGCGAATTCCAGCACACTGGCGGCCGTACTAGTG |
| | | GATCCGAGCTCGGACCCAACTTTGATGCAT |
| Insulin-like growth factor binding protein 1 | M89791 | TTGGCGAATTGGGCCCTCTAGATGCATGCTCGAGCGGCCGCCAGTGTGATGGATATCTGCAGAATTCGCCCTTCGCG |
| | | GGATCCGGAGCCTCGACCTCTGCATGCCCTCACCCGTGGCCAGGGAGCCTGTGTACTAGAACCTGCCGCACCCGCCA |
| | | CGAGCAGCTTGTCCGGTTCTCAGCATGAAGAGGCAAAGGCTGCTGTGGCCTCTGAGGATGAGCTTGCCGAGAGCCCA |
| | | GAGATGACAGAGGAACAGCTGCTGGATAGCTTCCACCTCATGGCCCCATCCCGTGAGGACCAGCCCATCCTGTGGAA |
| | | TGCCATTAGCACCTACAGCAGCATGCGGGCCCGGGAGATCACTGACCTCAAGAAATGGAAGGAGCCCTGCCAACGGG |
| | | AACTCTATAAAGTGTTAGAGAGATTAGCTGCCGCTCAACAGAAAGCAGGAGATGAGATCTACAAATTTTATCTGCCAAA |
| | | CTGCAACAAGAATGGATTTTATCACAGCAAACAGTGCGAGACATCTCTGGATGGAGAAGCTGGGCTCTGCTGGTGTGT |
| | | CTACCCATGGAGTGGGAAGAAGATCCTTGGATCAAGCTTGGCCAAGGGCGAATTCCAGCACACTGGCGGCCGGTACT |
| | | AGTGGATCCGAGCTCGGTACCAACTTGATGCATAGCTTGAGTATTCTATAGTGNCACCTAAATAGCTTGG |
| Insulin-like growth factor binding protein 3 | M33300 | TGCGAATTGGCCCTCTAGATGCATGCTCGAGCGGCCGCCAGTGTGATGGATATCTGCAGAATTCGCCCTTCGCGGGAT |
| | | CCCTGAAGGCGCTGCTGAATGGCCGCGGGTTCTGCGCCAACGCCAGCGCCGCCAGCAACCTGAGTGCCTACCTCCC |
| | | CTCCCAGCCGTCTCCTGGAAACACCACTGAGTCTGAGGAGGACCACAATGCTGGGAGTGTGGAAAGCCAGGTTGTCC |
| | | CCAGCACACATCGCGTGACTGATTCCAAGTTCCATCCACTCCATTCAAAGATGGAGGTCATCATAAAAGGCCAGGCTA |
| | | GGGACAGCCAGCGCTACAAAGTTGACTATGAGTCCCAGAGCACAGACACCCAGAACTTCTCCTCCGAGTCTAAGCGG |
| | | GAGACAGAATATGGTCCCTGCCGCAGAGAAATGGAGGACACACTGAATCATCTGAAGTTCCTCAATGTGCTGAGTCCC |
| | | AGGGGCGTCCACATCCCAAACTGTGACAAGAAGGGGTTCTATAAGAAGAAACAGTGTCGCCCTTCCAAAGGCAGAAAG |
| | | CGGGGCTTCTGCTGGTGCGTGGACAAGTACGGGCAGCCATTGAAGCTTGGCCAAGGCGAATTCCAGACACTTGGCGG |
| | | CCGTTCTAGTGGATCCGACTCGGTACCCAACTTGATGCATAGCTTGAGTATTCTATATGTCACCTAA |
| Insulin-like growth factor binding protein 5 | M62781 | GAGAATTGGGCCCTCTAGATGCATGCTCGAGCGGCCGCCAGTGTGATGGATATCTGGAGAATTCGCCCTTATCGCGG |
| | | GATCCTGCCGCAGTCACATGGAAGCTTCCCTCCAGGAGTTCAAAGCCAGCCCACGCATGGTGCCCCGTGCCGTGTAC |
| | | CTGCCCAACTGTGACCGCAAAGGATTCTACAAGAGAAAGCAGTGCAAGCCTTCTCGTGGCCGCAAACGTGGCATCTGC |
| | | TGGTGTGTGGACAAGTATGGGATGAAGCTGCCGGGCATGGAGTACGTCGATGGGGACTTTCAGTGCCACGCCTTCGA |
| | | CAGCAGTAACGTTGAGTGACGCGTCCCCTCCCTTCCTCCCCCTTTCCTACCCCCCAGCCCCAACTCCAGCCAGCGCCT |
| | | CCCTCCACCCCAGGACGTCACTCATTTCATCTCATTTAGGGGAAATATATATACATATATATTTGAGGAAACTGAGGACC |
| | | TCGGAATCTCTAGCAAGGGCTAAGGAGACACTCCCCATCCCGACCCCGGAAACGTATTCCTATTTGAAGCAAGTTGAA |
| | | CGGACAGAGAAGGGAAGAAGAGAAGGGGCAAGAAGGAGCGACTCGAGGGCCAAAAGGGCGAATTCCAACACACTGG |
| | | CGGNCGTTACTAGTGGATCCGAGCTCGGTACCAAACTTTGATGC |
| Insulin-like growth factor I | M15480 | CGAATTGGGCCCTCTAGATGCATGCTCGAGCGGCCGCCAGTGTGATGGATATCTGCAGAATTCGCCCTTCGCGGGAT |
| | | CCTCTTCACATCTCTTCTACCTGGCACTCTGCTTGCTCACCTTTACCAGCTCGGCCACAGCCGGACCAGAGACCCTTTG |
| | | CGGGGCTGAGCTGGTGGACGCTCTTCAATTCGTGTGTGGACCAAGGGGCTTTTACTTCAACAAGCCCACAGGCTATG |
| | | GCTCCAGCATTCGGAGGGCACCACAGACGGGCATTGTGGATGAGTGTTGCTTCCGGAGCTGTGATCTGAGGAGGCTG |
| | | GAGATGTACTGTGCTCCGCTGAAGCCTACAAAGTCAGCTCGTTCCATCCGGGCCCAGCGCCACACTGACATGCCCAA |
| | | GACTCAGAAGTCCCAGCCCCTATCGACACACAAGAAAAGGAAGCTGCAAAGGAGAAGGAAAGGAAGCTTGGCCAAGG |
| | | GCGAATTCCAGCACACTGGCGGCCGTTACTAGTGGATCCGAGCTCGGTACCAAGCTTGATGCATAGCTTGAGTATTCT |
| | | ATAGTGTCACCTAAATAGCTTGGCGTAATCATGGTCATAGCTGTTTCCTGTGTGAAATTGGTATTCCGCTCACAATTCAC |
| | | ACAACATACGAGCCGGAAGCATAAAAGTGTAAAAGCCTGGGGTGCCTAA |
| Insulin-like growth factor I, exon 6 | S43941 | TATGACATGATTACGAATTTAATACGACTCACTATAGGGAATTTGGCCCTCGAGGCCAAGAATTCGGCACGAGGATCCT |
| | | ATTCTCCAAAGACTATCAAGTGCTCGAAAACATTGTAAAATGAGTCTACTCCACTGAAAAAGTTAATGCATCTCTCCATT |
| | | ATAATCTATTTTCCTGCGGTTAAAAAAAAATCCTTTTTTAAATACCCACACACACACACACACACACACACCTATTCACCC |
| | | TAAGAGTTAGAAGAAATCATTTCCTTGAAAAATCCTATCTTTAAAAAAAAAAAAAAATCGACCCATGCCGCTTGAGACTC |
| | | CTTCTCCTTCTTTGAATGTCAATGTAAAACGTGGTTATGTCTGTAGATGAAACCGTCTCAGACGCCACGGCTCCAGGGT |
| | | TTCTGTTACAGTTTGTGCACTTGGGAGGATGCGCAGAAGACAGGACGCAGCGTGTTTTGCTTTCCCCTTTACTGTTTGG |
| | | CCAGCTACGCCAATGTGGTGCTATTGTTTCTTTAAGAAAGTACTTGACTTAAAAAAAANNNGAANAANANNAANAAAAAA |
| | | CCTTTGCGGCCGCAAGCTTATTCCCTTTAGTGAGGGTTAATTTTAGCTTGGCACTGGCCGTCGTTTTACAACGTCGTGG |
| | | ACTGGGAAAACCCTGGCGTTACCCAACTTAATCGCCTTGCAGCACATCCCCCTTTCGCC |
| Integrin beta1 | U12309 | TTGTGAATTGGCCCTCTAGATGCATGCTCGAGCGGCCGCCAGTGTGATGGATATCTGCAGAATTCGCCCTTCGGGATC |
| | | CGCTTGTAAGTGCACAGATCCCAAGTTCCAAGGGCCAACCTGTGAGACCTGTCAGACCTGCCTTGGTGTCTGTGCAGA |
| | | GCATAAAGAATGTGTTCAGTGCAGAGCCTTCAATAAAGGAGAAAAAAAAGACACGTGTACACAGGAGTGCTCCCATTTC |
| | | AACCTCACTAAAGTGGAAAGCAGGGAGAAGTTGCCCCAGCCTGTGCAGGTGGACCCTGTGACCCACTGCAAGGAGAA |
| | | GGACATTGATGACTGCTGGTTCTATTTCACCTACTCAGTGAACAGCAACGGTGAAGCTCACGTGCATGTTGTGGAGACT |
| | | CCAGACTGTCCTACTGGTCCCGACATCATCCCAATTGTAGCAGGCGTGGTTGCCGGAATTGTTCTTATTGGCCTTGCCT |
| | | CGCTGCTGATTTGGAAACTTTTAATGATAATTCATGACAGGAGGGAATTTGCTAAATTTGAAAAGGAGAAAATGAATGCC |
| | | AAGTGGAACACGGGTGAAAATCCTAAGCTTGACCAAGGGCGAATTCCAACACACTGGCGGCCGTTACTAGTGGATCCG |
| | | AGCTCGGTACCAAACTTTGATGCATACTTGAGTATTCTATANTGTCACCTAAATAGCTTGGCGTAATCN |
| Interferon related developmental regulator | NM_019242 | AACATGATTACGAATTTAATACGACTCACTATAGGGAATTTGGCCCTCGAGGCCAAGAATTCGGCACGAGGGTGATGAT |
| IFRD1 (PC4) | | GTAAACATGAGAATTGCTGCTGGCGAATCTTTGGCACTTCTGTTTGAATTGGCCAGAGGAATGGAGAGTGAACGGGAT |
| | | TTTCCAACAGAAACTGTTAAATTCGGTCCTGAGCGCATGTATATTGATAGCTGGGTCAAAAAGCACACCTATGACACGT |
| | | TTAAAGAGGCTCTTGGATCAGGGATGCAGTACCACTTGCAGACAAATGAATTCCTTCGCAATGTATTTGAGCTGGGACC |
| | | CCCTGTGATGCTCGATGCTGCAACACTTAAAACCATGAAGATTCCTCGTTTTGAAAGGCATTTATATAACTCTGCAGCTT |
| | | TCAAAGCTCGAACAAAAGCCCGAAGCAAATGCCGAGATAAGAGAGCAGATGTTGGAGAATTCTTCTAGATGTCTGACTT |
| | | TGATGTCTGTTTTCTAATTTCTTCCTTTATTATTATTTTTGCTACTTCTAATGTATATAAGCTTTTAGAGACTTTTTTTATCTT |
| | | GGTCAACTTAGATAATTTTTGATGTANGGATGGGTTATATTTTAATTTAATGNACAGTGGTACAAATTAATGAGTTCTTTA |
| | | TTCTGTAAAAATAACTGATNACCACAAATNAAAGTGGTTGTGGATGC |
| Interleukin-18 | AJ222813 | GCGAATTGGGCCCTCTAGATGCATGCTCGAGCGGCCGCCAGTGTGATGGATATCTGCAGAATTCGCCCTTATCGCGG |
| | | GATCCTGGCTGCAATACCAGAAGAAGGCTCTTGTGTCAACTTCAAAGAAATGGTGTTTATTGACAACACACTTTACCTTA |
| | | TACCTGAAGATAATGGAGACTTGGAATCAGACCACTTTGGCAGACTTCACTGTACAACCGCAGTAATGCGGAGCATAAA |
| | | TGACCAAGTTCTCTTCGTTGACAAAAGAAACCCGCCTGTGTTCGAGGACATGCCTGATATCGACCGAACAGCCAACGA |
| | | ATCCCAGACCAGACTGATAATATATATGTACAAAGATAGTGAAGTAAGAGGACTGGCTGTGACCCTATCTGTGAAGGAT |
| | | GGAAGGATGTCTACCCTCTCCTGTAAAAACAAAATCATTTCCTTTGAGGAAATGAATCCACCTGAAAATATTGATGATAT |
| | | AAAAAGTGATCTCATATTCTTTCAGAAACGTGTGCCAGGACACAACAAAATGGAATTTGAATCTTCCCTGTATGAAGGAC |
| | | ACTTTCTAGCTTGCCAAAAGGAAGATGATGCTAAGCTTGGCCAAGGGCGAATTCCAGCACACTGGCGGCCGTTACTAG |
| | | TGGATCCGAGCTCGGACCAAGCTTGATGCATAGCTTGAGTATTCTATAGTGCACCTAAATAGCTTGG |
| Iron-responsive element-binding protein | NM_017321 | TATGACATGATTACGAATTTAATACGACTCACTATAGGGAATTTGGCCCTCGAGGCCAAGAATTCGGCACGAGGGGGA |
| | | AGACCTTCCAGGCCGTGATGAGGTTCGACACCGATGTGGAGCTCACTTACTTCCACAATGGAGGCATCCTGAACTACA |
| | | TGATCCGAAAGATGGCCCAGTAGGTGCTGGCCTCTCAGGAGACCCGCGCTTGGTGCTAGACCCAATGAGGTACCAGG |
| | | CCTCCGCTGGTGGAGGCCTGGCGAGCAGCCACCTCTACTTCTCGTGAGGGTGCTAGCAAGATGAGCAAGTGGGCCCT |
| | | GCCATTCCTGGAGGCTCAGCGGCAGGAGTCTCTAGTTCGGTGATTTGTTAATCTTTTTATCCTTTTCTGTAATCCGGAAT |
| | | CTAGAATCATGGGAAGGTCCATAGTCCCAAAGAGAGCTACCTTCTCTTTAAAGTCACTCATCACCGGTCATTGATTTTTT |
| | | TCACTCTGACTAATCTTCAGCAGAACTAGCCAGTATCTCAGAAGTGTCTCCTACCCTTTCTGTTACTCTGTCTGTCTGTG |
| | | CTCAGTGACACCCTTCCCTGGAGAGCCCATTCCTCCGTGTATCACACCAATGGTAACGACATAGCTTCAGACTCTGTCA |
| | | CACTTCAATTCATAGTAATCGNGTGATCCCTTNCTTCCAAGTGAGCGAAAAACCTTGTGGCTAAGGGCG |
| JNK1 stress activated protein kinase | L27129 | GCGAATTGGGCCCTCTAGATGCATGCTCGAGCGGCCGCCAGTGTGATGGATATCTGCAGAATTCGCCCTTAACAGCTC |
| | | GGAACACCTTGTCCTGAATTCATGAAGAAACTACAACCAACAGTAAGGACTTACGTTGAAAACAGACCTAAGTATGCTG |
| | | GCTATAGCTTTGAGAAACTGTTTCCTGATGTGCTTTTCCCAGCTGACTCAGAACATAACAAACTTAAAGCCAGTCAGGC |
| | | GAGAGATTTGTTATCTAAAATGCTGGTGATAGATGCGTCCAAAAGGATCTCCGTAGACGAAGCTCTCCAGCACCCGTAC |
| | | ATCAACGTCTGGTATGATCCTTCAGAAGCAGAGGCCCCACCACCAAAGATCCCTGACAAGCAGTTAGATGAAAGGGAG |
| | | CACACAATAGAGGAGTGGAAAGAACTGATATACAAGGAGGTCATGGATTTGGAGGAGCGAACTAAGAATGGCGTCATA |
| | | AGAGGGCAGCCGTCTCCTTTAGGACCAGNGCAGATCANTGGCTTNGATCNTACGATGCGTTCGCNGNNTGGGAATGA |
| | | CATGACNTGNATGTCCAGAGNACCGANTCTGG |
| Lecithin:cholesterol acyltransferase | NM_017024 | TTATGACATGATTACGAATTTAATACGACTCACTATAGGGAATTTGGCCCTCGAGGCCAAGAATTCGGCACGAGGATGG |
| | | GTATGTGCGGGATGAGACAGTGCGGGCTGCACCCTATGACTGGCGTCTGGCACCCCGCCAGCAGGATGAATACTACC |
| | | AGAAGCTGGCAGGACTGGTAGAGGAGATGTATGCTGCTTACGGGAAGCCTGTTTTCCTTATTGGACACAGCCTTGGCT |
| | | GCCTACATGTGCTCCATTTCTTACTGCGTCAGCCACAGTCCTGGAAGGACCACTTCATTGATGGCTTCATCTCTCTTGG |
| | | GGCCCCATGGGGCGGTTCCATCAAGCCCATGCGGATCCTGGCCTCAGGTGACAACCAGGGCATCCCGATCATGTCCA |
| | | ACATAAAGCTGAGAGAAGAACAGCGCATAACCACAACTTCCCCCTGGATGTTTCCAGCTCACCACGTGTGGCCTGAAG |
| | | ACCACGTGTTCATTTCCACACCAAACTTCAACTACACAGGCCAAGACTTCGAGCGTTTTTTTGCAGATCTTCATTTTGAA |
| | | GAAGGCTGGCACATGTTTCTACAGTCTCGTGACCTACTAGCAGGCCTCCCAGCGCCTGGTGTAGAAATATATTGTCTAT |
| | | ACNGTGTAAGGCATGCCCACAGNCCACACCTACATCTATGACCACNACTTTCCTACAAAGACCCGGNGGCTGCACTC |
| L-gulono-gamma-lactone oxidase | D12754 | TATGACATGATTACGAATTTAATACGACTCACTATAGGGAATTTGGCCCTCGAGGCCAAGAATTCGGCACGAGGCACAA |
| | | GCACAATGGGAACTGACCTCTCCTCCTGACCACAAAGAAAGGCTGGGCTCTGGGCGGGTCCTCTCTGCCTTCGGCAT |
| | | CATTTCCCTTACATCCAGGCAAGAAGTGGCCTCTCACTCAAATTCCTGTTAGCATTTCCATGGGTCACACATAAACTGCA |
| | | ATCCTCTCAGGAGAAGGGGGATCCCTGATACATCATATCTATCCAGACTAAGGATGTGGTTCTTCCTAGATTCTATGGC |
| | | TCCACCAGGTATAGAGAGATTCCTGGGGCCTGCAGTTCTCCATCCCTCTTCAGAAGGGAGGGATCCCTTGGCGAGAGT |
| | | TTGGCTCAGAGGTGGCATGAAGCATGCTCTGCTCTCTCTTACCCTTGAAGGTCCTTCGGATGCCCAGAGATGTCTGCT |
| | | GGTCCTGGGCAAGCCATCATTCAAACGGGTCCAACCTGGCCTTCTGTCTGCCATGGCCTGACCCTCGCAGTGTCTCTT |
| | | CCAGAGGTGTTTAGAGTGGAACTCGCTTCAACCTCTTAACCAGTTGCTGATCCCTGTGTTTCTCTCCCTTCTCCTTGGA |
| | | GACTACTCTTGGAGGGGGATCCCACCATGTCCTTGGCTTTCCCTGGGTATTGTTCTCCTC |
| Liver fatty acid binding protein | V01235 | TNGNNATTGGGCCCTCTAGATGCATGCTCGAGCGGCCGCCAGTGTGATGGATATCTGCAGAATTCGCCCTTAGTACCA |
| | | AGTGCAGAGCCAAGAGAACTTTGAGCCCTTCATGAAGGCGATGGGTCTGCCTGAGGACCTCATCCAGAAAGGGAAGG |
| | | ACATCAAGGGGGTGTCAGAAATCGTGCATGAAGGGAAGAAAGTCAAACTCACCATCACCTATGGGTCCAAGGTGATCC |
| | | ACAATGAGTTCACCTTGGGGGAGGAGTGCGAACTGGAGACCATGACTGGGGAAAAGGTCAAGGCAGTGGTTAAGATG |
| | | GAGGGTGACAATAAAATGGTGACAACTTTCAAAGGCATAAAGTCCGTGACTGAATTCAATGGAGACACACAATCACCAATA |
| | | CCATGACACTGGGTGAAGGGCGAATTCCAGCACACTGGCGGCCGTTACTAGTGGATCCGAGCTCGGTACCAAACTTG |
| | | ATGCATAGCTTGAGTATTCTATAGTGTCACCTAAATAGCTTGGCGTAATCATGGTCATAGCTGTTTCCTGTGTGAAATTG |
| | | TTATCCGCTCACAATTCCACACAACATACGAGCCGGAAGCATAAAGTGTAAAGCCTGGGGTGCCTAATGAGTGAGCTA |
| | | CTCCATTAATTGCGTTGCGCTCCTTGCCCTTTTCAGTCNGGAAACCTG |
| Macrophage inflammatory protein-1 alpha | U22414 | GCGAATTGGGCCCTCTAGATGCATGCTCGAGCGGCCGCCAGTGTGATGGATATCTGCAGAATTCGCCCTTATCGCGG |
| | | GATCCTGGCGCTCTGGAACGAAGTCTTCTCAGCGCCATATGGAGCTGACACCCCGACTGCCTGCTGCTTCTCCTATGG |
| | | ACGGCAAATTCCACGAAAATTCATTGCTGACTATTTTGAGACCAGCAGCCTCTGCTCCCAGCCGGGTGTCATTTTCCTG |
| | | ACCAAGAGAAACCGGCAGATCTGCGCTGACCCCAAAGAGACCTGGGTCCAAGAATACATCACTGAGCTGGAACTAAAT |
| | | GCCTGAGATTAGAGGCAGCAAGGAACCCCCAAACCTCCGTGGGCCCCGTGTAGAGCAGGGGCTTGAGCCCCAGAAC |
| | | ATTCCTGCCACCTGCAAATCTCCCCCTCCTATAAGCTGTTTGCTGCCAAGTAGCCACATCCAGGGACTCTTCACTTGAA |
| | | TTTTTATTTAATTTAATCCTATTGATTTAATACTATTTAATTTTTTAATTTATTTTATTGNCACATTTGTGTTTGTAGCTATTT |
| | | ATTCTGAAAGACCTCAGGGCACATTCCTCAGCCCTCCCCCAAGCTTGGCCAACAANGGCGAATTCCAGCACACTGGCG |
| | | GGCGGTACTAGTGGGATCCCGACTCGG |
| Macrophage inflammatory protein-2 alpha | U45965 | CCGCCAGTGTGTGGAATTCGCCTTTGNCGGATCCGCCAGCTCCTCAATGCTGTACTGGTCCTTGCTCCTCCTGCTTGC |
| | | CCACCAACCATCAGGGACAGGTGAGACTCGAGGCTGACATTCTTGGAGGAGCCTCAGGTGGGCGCAGCCATGCCCA |
| | | GGCCCTCTGACCCACTCTCTTCTCCTACAGGGGTTGTTGTGGCCAGTGAGCTGCGCTGTCAATGCCTTGACGACCCTA |
| | | CCAAGGGTTGACTTTCAAGAACATCCAGAGCTTGACGGTGACCCCTCCAGGACCCCACTGCGCCCAGACAGAAGTCAT |
| | | AGCCACTCTCAAGGATGGTCATGAAGTTTGTCTCAACCCTGAAGCCCCCTTGGTTCAGAGGATCGTCCAAAAGATACTG |
| | | AACAAAGGCAAGGCTAACTGACCTGGAAAGGAAGAACATGGGCTCCTGTACCTCAACGGGCAGAATCAAAGAGAAAA |
| | | GAAACAAACTGCACCCAGGAAGCCTGGATCGTACCTGATGTGCCTCGCTGTCTGAGTTTATCTATTTATTTATATATGTA |
| | | TTTATTTATTTATTTTCAGTGCCTAGATGTTGTTACATTTACTATGATATTTAAAGATATGCATTGGCCAGCTCACTGTAAA |
| | | GCTTGGCCAAGGGCGAATTCTGCAGATATCCATCACACTGGCGGCCGCTCGAGCAGCATTNAGNGATCTAANGGCCA |
| | | TNCAA |
| MAP kinase kinase | D14591 | ATGCCGAATTGGGCCCTCTAGATGCATGCTCGAGCGGCCGCCAGTGTGATGGATATCTGCAGAATTCGCCCTTATCGC |
| | | GGGATCCGTCGCCTGAGAGACTCCAGGGGACTCACTACTCTGTGCAGTCGGACATCTGGAGCATGGGGCTCTCTCTG |
| | | GTGGAGATGGCAGTTGGAAGATACCCCATTCCTCCTCCTGATGCCAAGGAGCTGGAGCTGCTGTTTGGATGCCAGGT |
| | | GGAAGGAGACGCGGCCGAAACGCCACCCAGGCCAAGGACCCCTGGGAGGCCCCTCAGCTCATATGGAATGGATAGC |
| | | CGACCTCCCATGGCAATTTTTGAGTTGTTGGATTACATCGTCAATGAGCCTCCTCCAAAACTGCCCAGTGGAGTATTCA |
| | | GTCTGGAATTTCAGGATTTTGTGAATAAGTGCTTAATAAAGAACCCTGCAGAGAGAGCAGATCTGAAGCAGCTCATGGT |
| | | ACATGCTTTCATCAAGAGATCTGATGCCGAGGAGGTAGACTTCGCAGGCTGGCTCTGCTCCACCATTGGGCTTAACCA |
| | | GCCCAGCACACCAAGCTTGGCCAAAAGGGCGAATTCCAGCACACTGGCGGCCGTTACTAGTGGATCCGAGCTCGGTA |
| | | CCAAGCTTGATGCATAGCTTGAGTATTCTATAGTGTCACCTAAATAGCTTGGCGTAATCATGGCATAGCTGN |
| Matrin F/G | NM_022667 | TATGACATGATTACGAATTTAATTTAATACGACTCACTATAGGGAATTTGGCCCTCGAGGCCAAGAATTCGGCACGAGGAAAGT |
| | | CTTAAGGCCTGAAGTACACTGAGCTGAATGAGCACCGGGCCTGAGAGTTTAGTTTCTCCAAGTCCTTGGAAGGTATCC |
| | | CCAGCGTAGGCCCTACGTCCTCCAGACAAGATGCCCATAATGAGGCGGCCTCTGTTTTCACCAGTGTCTCAGGAATAC |
| | | TTAATGGAGTGAAAAGAGGGAGTCTTGCCTTCTGGGCCAGGCAGCCCGGATCTCCTCTGCCTCTGCCCACACCCAGG |
| | | AGAGCCAGAGGAGAAGCAGGTAGTTGGTTTCTTATCAGCTCCAGCGGGGCTAAGGGAGCTGGCTGTGTCCACTTTTCA |
| | | TCTGGATTCCGTCTAGCATGAAAGCCGTGCCCTCGAGGCTGTTTTGGAAACCACCATTTTGGGAAGTATCCCTCTCTAT |
| | | AAACTATGCCCCGGTATCTGAGGAGGAATGAAGGGACCAACAAGGCTGGATCATCCAAAACTGTTCACAGGAACCTGA |
| | | GGCCTATCCTCCCGTCGGCATACGCTTCTTTCTGCCTTTCACCCAACAGAGTCACAAGGAACCACCTTAGAANGGCCA |
| | | GTCTNCCANCCTCACAGTGGAGACAAAGGCTTTTCCTTCAAANACACNAATGGCGATCTCT |
| Matrix metalloproteinase-1 | X91785 | TTGCGAATTGGGCCCTCTAGATGCATGCTCGAGCGGCCGCCAGTGTGATGGATATCTGCAGAATTCGCCCTTATCGCG |
| | | GGATCCGGTACTGGCAGTGGGCCTTGCTGTCTTCTTCTTTAGACGCCATGGGACACCCAAGCGACTGCTTTACTGCCA |
| | | GCGTTCACTGCTGGACAAGGTCTGACCCCCACCACTGGCCCACCCGCTTCTACCACAAGGACTTTGCCTCCTCCGAAG |
| | | GCAGTGGCAGCCGGTGGTGGCAGGTGGGCTGTTCTCACCCATCCTGGGCTCCCTCCCTCCAGCCTCCCTTCTCAGTC |
| | | CCTAATTGGCTTCTCCCACCCTCACCCCAGCCTTGCTTCATCCATAGGTGGGTCCCTTGAGGGCTGAGCAGAAGATGG |
| | | TCTGGCCTCTGGCCCTCAAGGGACCCTCATAGCTTGGTGTGTGTCCAACCCTATTTGAATGTTGTCAAGGCTCTGCACT |
| | | TGAAGGCAGGACCCTCTGACCTTACAGGCAAAGGCCAAATGGGGTCATCTGCTTCTCTTCCATCCCCCTAACTACATAT |
| | | CTTAAATCTCTGAACTATGACCTCAGGAGGCTTTGGGAAGCTTGGCCAAAAGGGCGAATTCCAGCACACTGGCGGCCG |
| | | TTACTAGTGGATCCGAGCTCGGTACCAAC |
| Melanoma-associated antigen ME491 | NM_017125 | TCTCTATGACATGATTACGAATTTAATACGACTCACTATAGGGAATTTGGCCCTCGAGGCCAAGAATTCGGCACGAGGT |
| | | CTTAAGCTTCCACAGTAAGAATACCCCAACATGAACGCTGACCTCAAAAGCCACATTTCTGTGGCTGGGGTTTCT |
| | | GGGATTTTTCCTTCAAGATCCTGTCACAGGGCCAGGGAGATCGCTCAGTGGTAAAGCCCTAAGGGTGTAAGCTTGAGA |
| | | ACCCAAGTTTGACCCCCAGAACCCAGGAAAATGTGGAAGGAGAGAACCAACTTCACAATATTTTCCTTTGGTTTCTATA |
| | | CTCATGCCACAGTGCACCCTCTCTCCTAGATTGTGTGTACATGCAGAATTACCTTACAGACAACAAAACGGCCACCATT |
| | | TTGGACAAATTGCAGAAGGAAAATAAATGCTGTGGAGCCTCTAACTACACAGACTGGGAGCGCATCCCCGGTATGGCC |
| | | AAGGACAGGGTCCCCCGATTCTTGCTGCATCAACATCACTGTGGGGCTGTGGAAACGATTTCAAGGAATCCACCATCC |
| | | ATACCCAGGGCTGCGTGGAAACTATAGCCGGCATGGCTGANGAAGAACGTACTGCTGGTGGCTGGAGCAGCCCTGG |
| | | GCATTGCTTTTGTGGAGGTCCTGGGCATTATCTTCTCCTGCTGTCTGGNGGAAGANTATCCCGGAGTGGCTACGAAGT |
| | | AATGG |
| MHC class I antigen RT1.A1(f) alpha-chain | X99767 | TCTATGACATGATTACGAATTTAATACGACTCACTATAGGGAATTTGGCCCTCGAGGCCAAGAATTCGGCACGAGGCAT |
| | | AAATGACATAGGTTTCCATGTTGGAGTCGGTGGATAGAGAAGGCTCCCATCTCTGGGTAAGCGGCTCAGGCAGCCCCT |
| | | CATGCTCCACACGGCATGTGTAATTCTGCTCCTTCCCAAGTGGCACCACCACAGATGCCCACTTCTGGAAGGTTCCATC |
| | | CCCTGCAGGCCTGGTCTCCACAAGCTCCATGTCCTGGGTCAGGTCCTCCCCATTCAACTGCCAGCTCAGGGAGATGTC |
| | | AGCAGGGTAGAAGCCCAGGGCCCAGCACCTCAGGGTGACATCACCTTCAGGTCTGGGGTGAAGGGTCACATGTGCCT |
| | | TTGGGGGATCTAAGCGCAGCAGCGTCTCCTTCCCGTGCTCCAGGTATCTGCGGAGCCACTCCACGCACGTGCCCTCC |
| | | AGGTAGGCCCTGAGTCTCTCTGCAACACCAGCCCGATCCCACTTGTTCCGGGTGATCTGTGCCGCAAAGTCCGCCGC |
| | | CGTCCACGTCTTCAGGTCTTCGTTCANGGCGATGTAATCGCGGCCGTCGTAAGCGTCCTGCCTTATTACCCGCGGAGG |
| | | AGGNTTCCCGTTCCGTTCCCACGTCACAAGCCATACATTCCTCTGGATGGGGGTGAAANNCCGCCCCTCGCTCTTGG |
| Monocyte chemotactic protein receptor (CCR2) | U77349 | GCGAATTGGCCCTCTAGATGCATGCTCGAGCGGCCGCCAGTGTGATGGATATCTGCAGAATTCGCCCTTATCGCGGG |
| | | ATCCTGATCAGCATACTTGTGGCCCTTATTTTCCAACAATCTGGAAGAATTTCCAAACAATAATGAGGAATATCTTGAGT |
| | | TTGATCCTGCCCCTACTTGTCATGGTCATCTGCTACTCAGGAATCCTCCACACCCTGTTTCGCTGTAGGAATGAGAAAA |
| | | AGAGGCATAGGGCTGTGAGGCTCATCTTTGCCATCATGATTGTCTACTTTCTCTTCTGGACTCCATACAATATTGTTCTC |
| | | TTCCTGACCACCTTCCAGGAATTCTTGGGAATGAGTAACTGTGTGGTTGACATGCACTTAGACCAGGCCATGCAGGTG |
| | | ACAGAGGCTCTTGGAATGACACACTGCTGCGTTAATCCTATCATTTACGCCTTTGTTGGTGAGAAGTCCCGAAGGTATC |
| | | TCTCCATATTTTTCAGAAAGCACATTGCCAAAAATCTCTGCAAACAATGCCCAGTTTTCTATAGGGAGACAGCAGACCC |
| | | GAGTGAGAAGCTTGGCCAAGGGCGAATTCCAGCACACTGGCNGGCCGNTACTAGTGGATCCGAGCTCGGNNCCCAN |
| | | CTTTGAN |
| Multidrug resistant protein-1 | M81855 | GAATTGGGCCCTCTAGATGCATGCTCGAGCGGCCGCCAGTGTGATGGATATCTGCAGAATTCGCCCTTCGCGGGATC |
| | | CTGGAACAGTGTTTCTAGATGGCAAAGAAATAAAGCAACTCAATGTCCAGTGGCTCCGCGCCCACCTGGGCATTGTGT |
| | | CCCAGGAGCCCATCCTGTTTGACTGCAGCATCGCCGAGAACATTGCCTACGGAGACAACAGCCGTGTCGTGTCTCATG |
| | | AGGAGATCGTGAAGGCAGCCAAGGAGGCCAACATCCACCAGTTCATCGACTCACTGCCTGAAAAATACAACACCAGAG |
| | | TGGGAGACAAAGGGACTCAGCTGTCGGGCGGGCAGAAGCAGCGCATCGCCATCGCGCGCGCCCTCGTCAGACAGCC |
| | | TCACATCTTACTTCTGGATGAAGCGACATCAGCTCTGGATACGGAGAGTGAAAAGGTCGTCCAGGAAGCGCTGGACAA |
| | | AGCCAGGGAAGGCCGCACCTGCATTGTGATCGCGCACCGCCTGTCCACCATCCAGAACGCAGACTTGATCGTGGTGA |
| | | TTCAGAACGGCCAGGTCAAGAACACGGCACAAGCTTGGCCAAGGGCGAATTCCAGCACACTGGCGGCCGTTACTAGT |
| | | GGATCCGAGCTCGGTACCAAGCTTGATGCATACTTGAGTATTCTATAGTGCACCTAAATACTTG |
| Multidrug resistant protein-2 | L15079 | ATCCCATGGCCGGACCAGTGTTCCTCGATGGTCAGNAAGCAAAGAAACTCAATGTCCAGGGTCCGAGCTCACTTTGGC |
| | | ATTGTGTCCCAGAAGCCCATCCTGTTTGACTGCAGCATCGCCGAGAACATCGCCTACGAGACANCAGCCGTGTCGTGT |
| | | CTCAGGATGAGATTGTGAGGCGGCCAAGGAGGCCAACATCCACCCCTTCATTGAGACACTGCCCCAAAAGTATGAAAC |
| | | AAGAGTAGGAGACAAGGGGACACAGCTCTCTGGAGGCCAGAAACAGAGGATTGCTATCGCCCGAGCCCTCATCAGAC |
| | | AGCCTCGGGTCCTACTGCTGGATGAAGCCACGTCGGCTTTGGACACTGAGAGTGAAAAGGTCGTCCAGGAAGCGCTG |
| | | GACAAAGCCAGGGAAGGCCGCACCTGCATTGTGATCGCGCACCGCCTGTCCACCATCCAGAACGCAGACTTGATCGT |
| | | GGTGATCGACAACGGCAAGGTCAAGGAGAAGCTTGGCCAAGGGCGAATTCTGCAGATATCCATCACACTGGCGGCCG |
| | | CTCGAGCATGCATCTAGANGCCCGGNTACNAGNAAGNNCAN |
| NADH-cytochrome b5 reductase | D00636 | TATGACATGATTACGAATTTAATACGACTCACTATAGGGAATTTGGCCCTCGAGGCCAAGAATTCGGCACGAGGAGACA |
| | | GAGAGAGAGGAGTCAGAGAGCTGTTAGTACCATGTCTACCCGTTACCATGGACCGCAATGTGTAGTAAGGAAATGAAA |
| | | GGTAAACAGTTATCTACAGTCCTAGGAACCATGCCTGTCACCTAACCACCTACTGCCTTAGGCTATGGCCTGGTCTGTG |
| | | CCCCTAAACACTACATTAGACAGTGACACCCAGAGGTTCTCTTAGGAGGGTGTCTGGCAGTGACAGGCCAGCCCCCAC |
| | | TTGCTGGCCAGGAGTTCCTTGGGTCAGCACGTGGACCCTTCCAAAACCCCACACACTGCCCCTCCCCTCCATGATGCC |
| | | TTGAGCACCCTAAGTTGTCAGCCCAACAGAGTTTGGTAGACCTTGGGGTACCTGGCTTGTTTCTCCATCCCATACCTCC |
| | | CTCCCATCTGGTTCCCAGCCTGGGGGGTTCTGAGCAGAGCCTCTTTTCTCGGAGACAGACCCGGTGCCTGGCGCTGC |
| | | CTTCAGCAGACAGCAGACAGCCTCCTGCACACTGGCTTTTTTTAGTCATTTATGGGCAAAATGAGTTAAAATAAAACTTT |
| | | GCAAATCCTGAANANNNANNNNNNNNNNNNNNNNNNNNCNTTTGCGGCCGCAAGCTTATTCCC |
| NADPH quinone oxidoreductase-1 | J02640 | GAGGACGCCNGGAACNTGNGCANANATNGNANNCNNAGNAAGAGCNNANNTANACNTANNNNCCTACTAAANANANA |
| (DT-diaphorase) | | NNTNGTTNTGAAANGAGCGGNANATTGCNAATCATNNCCNGTNNAAANGNAAAGNAAATATTGCANAANTTCACCNCATNATAG |
| | | ANGGGATCCNTGNTNTNGCNGGANTCTTAAANGANGAAAGGATGAGAAGGTTGNTCGAAATNTGCCCCTCTGANGCNT |
| | | ATCAACNTTTNGACCCCCCNTCANTTTCCAAGAAATCNANNATCAACAAGGGGAAGCCNAAAGGANTCCGNAGAACTTT |
| | | TCAGGTACNCCTGGTTGNNTTCATCATCNNGGCGGANAANGTAAGGCCCGCNTNAGNCCCGGATATTGTNGCTTANCA |
| | | GATACAAGCTGGANNCCTGCAGGGCCTGGNGANATTATCACGTNTCCCCTATTGNATTGGNTNGGGGTTGCCCNCCAT |
| | | TCTGANAANGCTGGTTTAANAGAGNGCTTGTAACNAGGATTNGCCTACATGTATNCCACCATGTATNACCAGGGTCCTT |
| | | TCCAGAATAAGAAGACCTTGCTTTCCATCNCCACCNGGGGGCNAGCGGNTCCATGTACTCTCTNGCAAGGGTGTCCAC |
| | | GGGGACATGAACTGTCATTCTCTGGCCAATTCAGAGTGGCATTCTGCGCTTCTGTGGCTTCCAGGTCTTANANCCTCAA |
| | | CTGGTGTACAGCATTGGCCACAACCCCACCCNATGCCCGCGTGCAGGTCNTGGAAGGGTGGAAGNAGCGTCTGGANA |
| | | NTGTCGGNAGGAGTCANCGNTNTANNTTGANGTCNANAATNCG |
| Neuropeptide Y | NM_012614 | TATGACATGATTACGAATTTAATACGACTCACTATAGGGAATTTGGCCCTCGAGGCCAAGAATTCGGCACGAGGTCCTC |
| | | GCAGAGGCGCCCAGAGCAGAGCACCCGCTGCGCAGAGACCACAGCCCGCCCGCCATGATGCTAGGTAACAAACGAA |
| | | TGGGGCTGTGTGGACTGACCCTCGCTCTATCCCTGCTCGTGTGTTTGGGCATTCTGGCTGAGGGGTACCCCTCCAAG |
| | | CCGGACAATCCGGGCGAGGACGCGCCAGCAGAGGACATGGCCAGATACTACATCAACAATCT |
| | | CATCACCAGACAGAGATATGGCAAGAGATCCAGCCCTGAGACACTGATTTCAGATCTCTTAATGAGAGAAAGCACAGA |
| | | AAATGCCACCAGAACAAGGCTTGAAGACCCTTCCATGTGGTGATGGGAAATGAAACTTGCTCTCCTGACTTTTCCTAGT |
| | | TTCCCCCCACATCTCATCTCATCCTGTGAAACCAGTCTGCCTGTCCCACCAATGCATGCCACCACCAGGCTGGATTCCG |
| | | ACCCATTTCCCTTGTTGTCGTTGTATATATGTGTGTTTAAATAAAGTATCATGCATTCAAAAAAAAAAAAAAAAAAAAACA |
| | | ATTGCGGCCGCAAGCTTATTCCCTTTAGTGAGGGTTAATTTTANCTTGGCACTGGCCGTCGTTTTACAACGTCGTGGAC |
| | | TG |
| NGF-inducible anti-proliferative putative | M60921 | TTATGACATGATTACGAATTTAATACGACTCACTATAGGGAATTTGGCCCTCGAGGCCAAGAATTCGGCACGAGGGCTA |
| secreted protein (PC3) | | GAGCCAGCCAGTCACCCTTAGTGAGGATCCCCTTCCCCATGTCTCTCCACTGCCGTGGCATCCCATGTCCTGGATTTC |
| | | TCAATTCCTCAGTTTCTACTCAAAGGTGCTACTTACCAAACACTCTGCCCGTCCCGCTCTCCCCAGCTTCGCACAGCCG |
| | | TCCCAGGTGGCTTCGTCTCTCCTGCTTTAAAGTTAACTTTGGGCCCACAGACCCGAGAGCTGTGGGTTGAAGCAAAGC |
| | | TGTGAATCGCTCCAGATGGTCCCTGTGTTCTGTCCACACACAGGTCCCCGCCTTTTTAGAAGCAGCCTCCTGGTCTCAT |
| | | GCTTAAATCTGTTCCTCACTGCCCGTGTTCACTTTAGAAATGGCAGAACCACAGAGCTGGACTGTTGAGCAGGCCTGTC |
| | | TCTCTCATTAAATAAAAATAAGTAAGTTTGTAAGCTATTCCGACAGAAGAGACAAAGGTTACTGATTGTACAATAGCGCT |
| | | TTTATATGGAAGACTGTACAGCTTTATGGACAAATGTAAAACTTTTTTGTTTTTAATAAAAATGTAGCAGACCAAAAAAAA |
| | | AAAAAAAAAACACATGCGGGCCGCAAGCTTATTCCCTTTTAGTGAGGGTTAATTTTACC |
| N-hydroxy-2-acetylaminofluorene | AI030692 | TATGACATGATTACGAATTTAATACGACTCACTATAGGGAATTTGGCCCTCGAGGCCAAGAATTCGGCACGAGGCTTAC |
| sulfotransferase (ST1C1) | | TATTACTTCTCAAGAATGAATAAAATGCTGCCTGACCCTGGTACCCTGGGAGAATACATTGAACAGTTCAAAGCTGGAA |
| | | AAGTGCTGTGGGGCTCCTGGTATGACCATGTAAAGGGATGGTGGGATGTGAAAGACCAACACCGTATTCTGTATCTCT |
| | | TCTATGAAGACATGAAAGAGGACCCTAAAAGAGAAATTAAGAAGATAGCAAAATTCCTGGAAAAAGACATATCAGAGGA |
| | | AGTTCTTAATAAAATCATCTACCACACCTCCTTTGATGTAATGAAGGAAAACCCAATGGCCAACTATACCACTCTACCCT |
| | | CCAGTATCATGGACCACTCTATATCTCCTTTCATGAGGAAAGGGATGCCTGGAGACTGGAAGAACTACTTTACTGTGGC |
| | | ACAAAGTGAGGATTTTGATGAAGACTACCGGAGGAAGATGGCAGGGAGCAATATTACCTTCCGCACAGAGATCTGAGA |
| | | GCAGTGAGGAAGAGGGAAGCCCTAGATTTCCTGACTATATGCTTTAGCTATTTGAGCTTCCATTCCTGAGTTTTGTATGT |
| | | CCTGNGATACTATTTCATCAAATGNAAATCAGACCTTCCACACTAGGTGATTATTCTTATTGATACC |
| NIPK | AB020967 | TATGACATGATTACGAATTTAATACGACTCACTATAGGGAATTTGGCCCTCGAGGCCAAGAATTCGGCACGAGGCCGG |
| | | CGTCCGGGTGCCGCAGCACTTTAGCAGCGGAAGCGGCTGCTGTTGAGTACGTGACGGCTGGGGTCTGGCACGGGCG |
| | | TGCTGGGACTCCGAGATAGGCTCAGTGAGGCTGGCAGCGGTCCACGGGCGCGGGCGCAGGCGGCTCTGAGACCCC |
| | | AGGACAAGATGCGAGCCACATCTCTGGCTGCTTCTGCCGATGTTCCCTGCAGGAAGAAACCGTTGGAGTTTGATGACA |
| | | ACATTGATGTCGAGTGTCCAGTCTTAAAACGAGTGAGAGATGAGCCTGAGCCTGGACCAACCCCCAGCCTACCTCCCG |
| | | CCTCAGACTTGTCACCTGCTGTGGCCCCTGCAACCCGGCTGGGGCCCTATATCCTTTTGGAACGAGAGCAAGGCAACT |
| | | GCACCTATCGGGCCCTGCACTGCCCCACAGGCACAGAGTACACCTGCAAGGTGTATCCCGCCAGTGAGGCCCAGGC |
| | | GGTGCTGGCACCGTATGCAAGACTGCCTACCCACCAGCACGTGGCCCGTCCCACAGAAGTCCTGCTGGGCTCTCANC |
| | | TCCTATACACCTTTTTCACGAANACCATGGGGACTTTGCACAGTCTGGTGCGCAACCGCCGCGGTATCCCCGAC |
| Nucleoside diphosphate kinase beta isoform | D13374 | GCGAATTGGGCCCTCTAGATGCATGCTCGAGCGGCCGCCAGTGTGATGGATATCTGCAGAATTCGCCCTTATCGCGG |
| | | GATCCGCCAACAGCGAGCGTACCTTCATCGCCATCAAGCCTGATGGGGTCCAGCGGGGGCTTGTGGGAGAGATCATC |
| | | AAGCGATTCGAGCAGAAGGGATTCCGCCTGGTTGGTTTGAAATTTATACAGGCTTCAGAGGATCTTCTCAAGGAGCAC |
| | | TACATTGACCTGAAGGACCGCCCCTTCTTTTCTGGCCTGGTGAAGTACATGCACTCAGGACCGGTGGTTGCTATGGTC |
| | | TGGGAGGGACTGAATGTTGTGAAGACAGGCCGGGTGATGCTTGGAGAGACCAACCCCGCAGACTCTAAGCCTGGGAC |
| | | CATACGAGGAGACTTTTGCATTCAAGTTGGCAGGAACATCATTCATGGCAGCGATTCTGTGGAGAGTGCGGAGAAGGA |
| | | GATCAGTTTGTGGTTTCAGCCTGAGGAGCTGGTGGACTACAAGAGCTGTGCGCAGAACTGGATCTATGAGTGACAGCA |
| | | GGGCCCAAGCTTGGCCAAAAGGGCGAATTCCAGCACACTGGCGGCCGTTACTAGTGGATCCGAGCTCGGTACCAAGC |
| | | TTGATGCATAACTTGAGTATTCTATAGTGTCACCTAAATAGCTTGGCGTAATCATGGTCATAGCTGGTTCCTGTG |
| Nucleosome assembly protein | AF062594 | TTCTGACATGATTACGAATTTAATACGACTCACTATAGGGAATTTGGCCCTCGAGGCCAAGAATTCGGCACGAGGCATC |
| | | TGGTTTCCATTCACATTCTTCTTCTGTAGGTTCATAAATTGCATTAATGATCTCAAATCGCTTATCAAACAGAGGCTGATA |
| | | GAGAACAGCATACTTTCTCTAAGGTCATGAACTTCCTCATAGAATTTGGCTTCTATCTGTGCACATTTAACTTGAAGAT |
| | | TCTTGAGAGCATTCACCCGTCTTTTGACTACCTTAGGCAAGCTTTCAATGTATCCTGTTGGTGTGTCTACCAGACCATCA |
| | | AGTCTTTCCTGAAGAGCTGCAAGAATCTGAGGATTTTGCATCATCTGAACAGTTAGCTGACGTGCTTTGATTTTTGTTTC |
| | | TTCACCCGTTTCTTCTTCTTCTACTTCTTCAACATCTTCCAAATCTTGATCAAGTTCAGACTGTTCTTTGTTGTCAATGTCT |
| | | GCCATCTTTTAAGAACTCCAAATATCGTTCCAGGAGGCTCGGACGCGGAGCCGAGCGGCCGGAGCTGCGCAGGCAGT |
| | | GACTCAAGGCGGCGGCGGCGGCGGCGGCGGCGGCGGCGGCNGGAGGACGGGANGCGCCCCCNAGCAGATGGCG |
| | | CTAAAAAAAAAAAAAAAAAAAAACCTTTGCGGCGCAGCTTTATCCCTTTAGNGAGGGGGTNATTTTAACC |
| Organic anion transporter 3 | AF041105 | TTGGCGAATTGGGCCCTCTAGATGCATGCTCGAGCGGCCGCCAGTGTGATGGATATCTGCAGAATTCGCCCTTCGCG |
| | | GGATCCGCATCCGGTCATCAGGAAATTCATCTGCAGTCCTGGGGTTATGTAAAAAAGGCCCTGAGTGTGCTAATAAACT |
| | | ACAGTACTTTTTAATCATGTCAGTAATTGGCAGTTTCATCTACTCGATCACAGCCATACCTGGGTATATGGTTCTTCTGA |
| | | GGTGTATCAAGCCTGAAGAGAAGTCGCTTGGGATTGGATTACATGCATTTTGCACAAGAGTATTCGCTGGCATTCCGG |
| | | CACCCATTTACTTTGGCGCTTTGATAGACAGAACCTGTTTACATTGGGGAACCCTGAAATGTGGTGAGCCCGGGGCAT |
| | | GCAGGATGTACAATATAAATAACTTCAGGCGCATTTACCTAGTGTTGCCTGCAGCTCTTAGAGGATCAGGCTATCTCCC |
| | | TGCACTCTTCATTCTGATACTTATGAGGAAATTCCAGTTCCCTGGGGAAATCGACTCTTCAGAAACTGAACTTGCAGAG |
| | | ATGAAGATCACAGTGAAGAAAAGTGAGTGCACAGATGTGCAAGCTTGGCCAAGGGCGAATTCCAGCACACTGGCGGN |
| | | CGTTACTAGTGGATCCGAGCTCGGACCAAC |
| Organic cation transporter 3 | AF055286 | GCGAATTGGGCCCTCTAGATGCATGCTCGAGCGGCCGCCAGTGTGATGGATATCTGCAGAATTCGCCCTTATCGCGG |
| | | GATCCCCTCTAGGTGGCGCCGTTCCAAGGAACCGTAAAGATTCCAGAGTATTTTATATGGAATAAGATAAAGATTTATC |
| | | ATGCTACCCAAGCTTCTGGGAACACATAACCTTTGACCTCTTTAGCAAAGATCCAGTAGGTACACAGATTCTGCCTAGG |
| | | TTCTCTTTCATTGGGACCAATGTCTCTGACCCACTGGGGAGGAGATATGAGAAGGTCCCAACCAGTTCTCCAGTTCTCA |
| | | GGACAGTCCCATCCAGGAAGCAGGATGAACATATCAAGGCCTGGCAGTGAACGGAAGTGTACTCAGCTGATCTGATCA |
| | | AGACTGAGCAGTGAGGAGTGTTCTGAAGAGTCTGAGAACTGCTATTTCTGATTCGCTTAAGCTCCGAATACATGCCTTC |
| | | AGAGAGCCGACATGCCTGGTGCCCCTCCCCTTCTGTCCCCAAAGCCACTGAGGTTCCAGGCCCCAGGTTAGGCACCT |
| | | CGAGGGCAAGGGCGAATTCCAGCACACTGGCGGCCGTTACTAGTGGATCCGAGCTCGGTACCAAGCTTGATGCATAG |
| | | CTTGAGTATTCTATAGTGTCACCTAAATAGCTTGGCGTAATCATGGNCATAGCTGTTTCCTGNGTGAAATTGTN |
| Ornithine decarboxylase | J04792 | TTTGAGAATTGGNCCCTCTAGATGCATGCTCGAGCGGCCGCCAGTGTGATGGATATCTGCAGAATTCGCCCTTATCGC |
| | | GGGATCCCATCGAGAACCAACCATGGGCAGCTTTACTAAGGAAGAGTTTGACTGCCATATCCTCGATGAAGGTTTCAC |
| | | TGCTAAGGACATTCTGGACCAAAAAATCAATGAAGTTTCTTCCTCTGATGATAAGGATGCTTTCTATGTTGCGGACCTCG |
| | | GAGACGTTCTAAAGAAGCATCTGAGGTGGCTGAAAGCTCTTCCCCGTGTTACTCCCTCCTATGCTGTCAAGTGTAATGA |
| | | CAGCAGAGCCATAGTGAGCACCCTGGCTGCCATGGGACAGGATTTGATTGTGCAAGCAAGACTGAAATACAGTTGGT |
| | | GCAGGGACTTGGGGTGCCTCCAGAGAGGATTATCTATGCAAATCCTTGTAAGCAAGTGTCTCAGATCAAGTATGCTGC |
| | | CAGTAATGGAGTCCAGATGATGACTTTTGACAGTGAAATTGAGTTGATGAAAGTTGCCAGAGCACATCCAAAGGCAAAG |
| | | TTGGTTTTTGCGGATTGCCACTGATGATTCCAAAGCAGTTTGTCGGCTCAGTGTTAAGTTTGGTGCCACACTGAAAACC |
| | | ANCAGGCTTCTCTTGGAACGGGCAAAAAGAGCTAAA |
| Osteoactivin | AF184983 | CNGGGTATGACATGATTACGAATTTAATACGACTCACTATAGGGAATTTGGCCCTCGAGGCCAAGAATTCGGCACGAG |
| | | GGCCGGCACTCTGACTCCTGGTGGATGGGACTAGGGAGTCAGAGTCAAGCCCTGACTGGCTGAGGGCGGGCGCTCC |
| | | GAGTCAGCATGGAAAGTCTCTGCGGGGTCCTGGTATTTCTGCTGCTGGCTGCAGGACTGCCGCTCCAGGCGGCCAAG |
| | | CGGTTCCGTGATGTGCTGGGCCATGAGCAGTATCCGGATCACATGAGGGAGAACAACCAACGTGGCTGGTCTTCA |
| | | GATGAAAATGAATGGGATGAACAGCTGTATCCAGTGTGGAGGAGGGGAGAGGGCAGATGGAAGGACTCCTGGGAAG |
| | | GAGGCCGTGTGCAGGCAGCCCTAACCAGTGATTCACCGGCCTTGGTGGGTTCCAATATCACCTTCGTAGTGAACCTGG |
| | | TGTTCCCCAGATGCCAGAAGGAAGATGCCAACGGCAATATCGTCTATGAGAGGAACTGCAGAAGTGATTTGGAGCTGG |
| | | CTTCTGACCCGTATGTCTACAACTGGACCACAGGGGCAGACGATGANGACTGGGAAGACAACACCAGCCAAGGCCAG |
| | | CACCTCANGTTCC |
| p53 | X13058 | TTGGCGAATTGGGCCCTCTAGATGCATGCTCGAGCGGCCGCCAGTGTGATGGATATCTGCAGAATTCGCCCTTCGCG |
| | | GGATCCAGGCAGACTTTTCGGCACAGCGTGGTGGTACCGTATGAGCCACCTGAGGTCGGCTCCGACTATACCACTATC |
| | | CACTACAAGTACATGTGCAACAGCTCCTGCATGGGGGGCATGAACCGCCGGCCCATCCTTACCATCATCACGCTGGAA |
| | | GACTCCAGTGGGAATCTTCTGGGACGGGACAGCTTTGAGGTTCGTGTTTGTGCCTGTCCTGGGAGAGACCGTCGGAC |
| | | AGAGGAAGAAAATTTCCGCAAAAAAGAAGAGCATTGCCCGGAGCTGCCCCCAGGGAGTGCAAAGAGAGCACTGCCCA |
| | | CCAGCACAAGCTCCTCTCCCCAGCAAAAGAAAAACCACTCGATGGAGAATATTTCACCCTTAAGATCCGTGGGCGTG |
| | | AGCGCTTCGAGATGTTCCGAGAGCTGAATGAGGCCTTGGAATTAAAGGATGCCCGTGCTGCCGAGGAGTCAGGAGAC |
| | | AGCAGGGCTCACTCCAGCTACCCGAAGACCAAAAGCTTGGCCAAGGGCGAATTCCAGCACACTTGGCGGCCGGTACT |
| | | AGTGGATCCGACTCGGTACCAAACTTGATGCATAGCTTG |
| p55CDC | U05341 | NACTCAAGCTATGCATCAAGCTTGGGACCCGAGCTCGGATCCACTTAGNACGCCCGCAGTGTGCTGNAATTCGCCCTT |
| | | CGCGGGATCCGTATGTGGGCTGCGCTGGGCCCCAGATGGACGACATCTGGCAAGCGGGTGGCAATGATAACATTGTC |
| | | AACATGTGGCCTAGTGGTCCTGGAGAAAGTGGCTGGGTTCCCCTGCAGACATTCACTCAACATCAAGGTGCTGTCAAG |
| | | GCTGTTGCATGGTGTCCCTGGCAGTCCAATATCCTGGCAACAGGAGGAGGTACCAGTGACCGACACATTCGCATTTGG |
| | | AACGTCTGCTCTGGAGCCTGTCTGAGTGCTGTGGATGTGCATTCCCAGGTGTGCTCCATCCTCTGGTCTCCCCACTAT |
| | | AAGGAGCTCATCTCAGGCCATGGCTTTGCCCAGAACCAGCTGGTTATTTGGAAGTACCCAACTATGGCCAAGGTGGCA |
| | | GAGCTCAAAGGTCACACAGCCCGGGTCCTGAGTCTCACCATGAGTCCAGACGGGGCCACAGTGGCATCTGCAGCAGC |
| | | CGATGAGACTCTGCGGCTCTGGCGCTGCTTTGAGCTGGACCCTGCCAAGCTTGGCCAAGGGCGAATTCTGCAGATAT |
| | | CCATCACACTGGCGGCCGCTCGAGCATGCATCTAGAGGGCCCAATTCGCAA |
| PAR interacting protein | U83590 | CTATGACATGATTACGAATTTAATACGACTCACTATAGGGAATTTGGCCCTCGAGGCCAAGAATTCGGCACGAGGGCTT |
| | | TCTTGGGCATGCTTCAAGGCAAGCAGCAGAAGCTGCAGCAAAACCTGCAGCAAGGGAATCACTCGTCTGGCTCCAGT |
| | | CGCCTCTACGATCTCTACTGGCAGGCCATGAACTTGCTTGGAGTGCAGCGTCCAAAGTCAGAAAAGAAGAATGTGAAG |
| | | GATATTCCTAGTGACAGCCAGAGTCCCATTAGCACAAAGCGGAAGAAAAAGGGATTCTTGCCAGAGACCAAGAAGCGA |
| | | AAGAAACTTAAATCTGAGGGTACCACATCAGAAAAGAAGGCTGCATCACAGCAGGATGCAGTGACAGAGGGTGCCATG |
| | | CCTGCTGCCACTGGTAAAGACCAGCCCCCCAGCACAGGCAAGAAAAGAAGGAAGAGAGTGAAGGCTAACACCCCATC |
| | | CCAGGTGAATGGAGTAACTGTGGCCAAGAGTCCGGCTCCCAATAACCCCACCCTGAGTCCCAGCACCCCCCCTGCCA |
| | | AGACCCCAAAAGTGCAGAAGAAAAAAGAGAAGCTGTCACAGGTGAATGGATCCACTCCTGTGTCCCCCCGTAGAGCCT |
| | | GAAAGCAAAAAGCATCAGAANGCTCTCAGCACAAAGGAGGTCAAAAAGAAAGTCCTCCAGTCTGGCCTGNCAAAAAAA |
| | | AANAA |
| Paraoxonase 1 | U94856 | TTGCGAATTGGGCCCTCTAGATGCATGCTCGAGCGGCCGCCAGTGTGATGGATATCTGCAGAATTCGCCCTTGTCCTG |
| | | GGAAATGTACTTGGGCCTGTCATGGTCCAATGTTGTATACTACAGTCCAGATAAAGTCCGAGTGGTAGCAGATGGATTT |
| | | GATTTCGCTAATGGCATTGGCATTTCCCTTGATGGCAAGTATGTCTATATCGCTGAATTGCTGGCTCACAAGATTCACGT |
| | | GTATGAAAAGCATGCTAATTGGACTTTAACGCCATTGAAGGTCCTCAGCTTTGACACCCTTGTGGATAACATCTCTGTG |
| | | GATCCTGTGACAGGGGACCTCTGGGTTGGATGCCATCCCAATGGGATGAGGATTTTTTTCTATGACTCGGAGAATCCT |
| | | CCTGGCTCAGAGGTGCTTCGGATCCAGAGCATTTTATCCGAAGACCCCAAAGTAACTGTAGTTTATGCAGAGAATGGC |
| | | ACGGTGTTGCAAGGTACGACAGTCGCTGCTGTGTACAAAGGAAAACTGCTGATTGGAACGGTGTTCCACAGAGCTCTT |
| | | TGTTGTAAGGGCGAATTCCAGCACACTGGCGGCCGTTACTAGTGGATCCGAGCTCGGTACCAAGCTTGATGCATAACT |
| | | TGAGTATTCTATAGTGNCACCTAAATAGCTTGGCGTAATCATGGTCATAGCTGGTT |
| Peroxisomal multifunctional enzyme type II | NM_024392 | TACATGATTACTACTTTAATACGACTCACTATAGGGAATTTGGCCCTCGAGGCCAAGAATTCGGCACGAGGCCTCGTGC |
| | | CGAGAGGACCCTGGGAGCCATTGTCAGGAAGCGGAATCAGCCCATGACTCCCGAGGCAGTGAGGGACAACTGGGTG |
| | | AAGATCTGTGACTTCAGCAATGCCAGCAAGCCGAAGAGCATTCAAGAGTCCACAGGTGGTATAATCGAAGTTTTACATA |
| | | AAATAGATTCAGAAGGAATCTCACAAAATCACACCGGTCAAGTGGCATCTGCAGATGCATCAGGATTTGCTGGCGTCGT |
| | | TGGCCACAAACTTCCTTCATTTTCTTCTTCATATACGGAACTGCAGTGCATTATGTATGCCCTCGGAGTAGGAGCTTCAG |
| | | TCAAAAATCCAAAGGACTTGAAGTTTGTTTATGAAGGGAGTGCTGACTTCTCCTGTTTGCCTACATTTGGAGTCATTGTG |
| | | GCTCAGAAGTCCTTGATGAGTGGAGGCTTAGCAGAGGTTCCTGGGCTGTCAATCAACTTTGCAAAGGTTCTTCATGGG |
| | | GAGCAGTACTTGGAGTTGTATAAGCCACTTCCCCGATCAGGGGAATTTAAAATGTGAAGCAGTTATTGCTGACATCCTG |
| | | GATAAAGGCTCTGGCATAGTGATTGTTATGGACGTCTATTCTTATTCTGGCAAGGACTTATATGCTATNATCAGTTC |
| Phase-1 RCT-102 | | TTATGACATGATTACGAATTTAATACGACTCACTATAGGGAATTTGGCCCTCGAGGCCAAGAATTCGGCACGAGGTGAA |
| | | GATGAAAAGCAAAACAACCTTTTTGATAAGCAAGGTATAGATTTTACATTTTTGTCCTTGCTCCCAATGAAATGGATAAA |
| | | CAAAAATAAAATCTGACAATGCCGTCTCTTCACGGAATGTTGTTGTGTTAGCCGGACTGAAAGCCCACCTTAATTTTTAT |
| | | ATAACGTCTTTAGCTCTTCCTTTGACAGGGCAGGCCTTGTTCTGAACTGTTTGCGCTTCTGACTGTTAGACACCAATGAC |
| | | GCATGCACTGTCCCTCTCATTCTCTCTTGCTTCCCCTCTGGCCTGAGTTTCTTGTGCATCCTCCCTACCCCCACCTCTGT |
| | | TAGGGTAGATATATCAGCTATGTAAATAGAGCAAGGAAACGGTATTGTGCATTTGTGGCATTTACGNAGAGTTGCAGTT |
| | | GTACGCTGCTGAAAACGCANGCTTTTTGTAACATGTGGTCCTTCCATAAGTACCCNATGTATTTTAGNCTATTTTAGTCG |
| | | TATTTGNTCNATAAAATATGCAAGCTATANGGTAAACANANAACAAANAAAAAAANACCNTTGCGGCCGCCAAGCTTATT |
| | | CCCTTTAATGAGGGGTTAAATTTTAGCTTGGGCCTGGCCGCCGTTTAACAANGNCC |
| Phase-1 RCT-107 | | TTATGACATGATTACGAATTTAATACGACTCACTATAGGGAATTTGGCCCTCGAGGCCAAGAATTCGGCACGAGGCTCA |
| | | AACTCACAATGTAGCCAAGACTGGCCAAGAGCGGACAAACCTGGCTCCACTTCCCAAGTGCTGGGATCATAGGAATGT |
| | | GCCACCACACCTCGGTGTTGACCTCAATCCCATAGTAAGCGGTGAGATTAACGTACCACAGAGCATTCCCATTGCGAT |
| | | CGCATTCTGCCATGAGGTGGGAAGAAAAATGGGCGATTATTTTTAGGGAAAGGACAAATTTGTGGTCACCTGATCTGAA |
| | | AGGACTGGAGATATTATTTTCCCTCAGGTGTTGGAGGACTGGTGGCTGATTGGTCAGACTCTTTCATGAACCGTGTAG |
| | | GAGCAGTGCTGATTGGTCAGGTTCATTTAAGAACCGCGGGAAGTGGTGCTGATTGGTCAGATTCATTTAAGAACCGCG |
| | | GGAAGTGGTGCTGATTGGTCAGATTCATTTAAGAACCGCGAGGAGCGGCGTTTGTTCCCACAACACATTTCATCCAGA |
| | | CGCAATGGAAAAAGCACTGTTAGCTTTCCTTGTTTGTGTGCTAAAGATTTGTTTTGCCTTATGATTGTTACTTTTGCTTGT |
| | | TTTGTTTGTCTTTGTAACAAAATGACTTTTTCCAAGGGAAAGGAACCTTTTAAGAAAAAGAACGGCACTCTCTGCACGAT |
| Phase-1 RCT-109 | | TCTCAAAGTGATTTTTCCCAAGNATACGACTCACTATAGGGAATTTGGCCCTCGAGGCCAAGAATTCGGCACGAGGGA |
| | | GAAGCTACTAGGCATGTCAAGCAGTCAGCAGGATTTGTCTTCATGGTCTTCTGCAGATGAGGTGGACACGTCGGGGTC |
| | | AGTGAGTCCTACATCAGGAAGAAGTACCCCAAACAGACAGAAATCGGTCCACAAAAGGTGGCTCCGATTCCTCCCTTT |
| | | CTAAACCAGGCCAGCACGGGCAGGTCGAGATTTCTTGTTCCGGGTCCTCAGGGCAGCTCTTCCGATTCAGCTGCTCCT |
| | | ACTACTCCTTATCGGACTCACCTCCCGTGTACCCATATCGGAAGAAGACTACAGCTGTGCCCTCTCCAACAACTTTGCC |
| | | AGATCGTTCCATCCCATGCTCAGATATACCAACGGTCCTCCTCCCCTCTGAAGCAAGCACACGTGCAGGCAGCAAGAG |
| | | GGGAAGCAGGTCCAAAGCAACCCCAGGCTGTCAGAGAAGGTGTGATCTTGGCAGAGGTCATCATATGGCAGCTTCCG |
| | | TTCTCCCTAACCCGTGTATAAAAAGAAGGAAAAAAAAGCCTTTGGGACATGGGACAGTAAAACAACAGGAGGCAAACA |
| | | AAACTTGTAATTGGTACAGTGNCNATTTGANACTCTGAGCCCTTAATACTAAAAGAAATTAAGGACATATANGGGTTCCC |
| | | TANAACCCATGAAGTCTTGGGCCC |
| Phase-1 RCT-110 | | TATGACATGATTACGAATTTAATACGACTCACTATAGGGAATTTGGCCCTCGAGGCCAAGAATTCGGCACGAGGCCTGC |
| | | AGTGAGAAAGTGGCAAAGGGCTTTTCAGCTAAGGTCACCCCAGTGACCACCATCCTTTGTCCACTGGGGCAGGTCTTG |
| | | CATGTGTCCCTAATGCCGCATGGGGTCCCTGCCTGCCATCTCCCATCTGACCCACCTCTGTCATGCATGGTGATGGTG |
| | | ATGTGTTTACTGTCTGGTCCTGTGCCTGTCTGAGACCGTCTCTGTGGAACTTGCCTTAAACTGAAGTAAACCAGTTCTTT |
| | | TAGTAAAACCCCTTCTCCTTGGAAAACAAAAAAAAAAAAAAAAAAAAAAACCAATTGCGGCCGCAAGCTTATTCCCTTTA |
| | | GTGAGGGTTAATTTTAGCTTGGCACTGGCCGTCGTTTTACAACGTCGTGACTGGGAAAACCCTGGCGTTACCCAACTTA |
| | | ATCGCCTTGCAGCACATCCCCCTTTCGCCAGCTGGCGTAATAGCGAANAGGCCCGCACCGATCGCCCTTCCCAACAGT |
| | | TGCGCANCCTGAATGGCAAATGGGACGCGCCCTGTANCGGCGCATTAACGCGGCGGGTGTGGTGGTTACGCGCAAC |
| | | GTGACGCTACATTTGCCAGCGNCCTANCGCCCGCTCCTTTCCTTTCTTCCCTTCCTTT |
| Phase-1 RCT-117 | | TTCCTGTTTCTAATTTGGGGGGTATNTACGGGAAGCATGTTAACAACCAAGCATTCAACCGTGNAGACACAGACTTCCA |
| | | GTCTCCCTAGCAGGCAGGAAACCCCGTTGTGTGCCTGATGGGTGTTTGCGATGTGCTGGTTGGTGTTTTACACTGTGA |
| | | TAATAATCCTTCTCGGCTTACATCTCCTTCTTTTTTGAAAGCCCATTTTTCCAGGCTTGAGGAGAAGGGCTTAGCGAACA |
| | | GACCACCTGTCACGCAAGCATGAGGACCTTAGTTCGAATCCCAGGCATTCACACACAAGCCAAGTATGGACCTAGCTA |
| | | ACCGGCCACACTTAACCCCTATACCCCTCTCACACTCACCTCTTTTCCAGCCTAGCGTGCGCCTCTCCCCCTAAGACCC |
| | | ATAAACAGTACAGAAAACCCAGAGAGAGCATGCTCTGAGGCACCGAGAGAGATTAGCCGTTTTCGCTGCTTTCAGTTC |
| | | ATCACATCAGCGTTCCAGCTTTACACCCGCCAGTATCTACGACTCACACCTACTGGACGCCAAGTCCCCCACGGCTGG |
| | | TCCAGTTCTCCATTCTCATCCTCAAG |
| Phase-1 RCT-12 | | CNTTATGACATGATTACGAATTTAATACGACTCACTATAGGGAATTTGGCCCTCGAGGCCAAGAATTCGGCACGAGGCT |
| | | GAATTCAGGTCACCTCAATGATAAGTCTTCGACTTGCCCCAATGTTAGTCCTGCATTCCTAGCTCTTAAGAGATGCAATG |
| | | ACACTTACTGGCTCATTCTTGTCAAGTTGGATGCGGTAGCGGGAGATGAAGGTGGGTTTTCCTGTTCTGTCCACCATCA |
| | | GTAGAAGGCCATTGAAGCCCCAGAAGCACAGCCCCGGCACGAGGACAACACCTGAGGCAGAGAGCAAAGGGACTTA |
| | | GAGCAGCCCCAGGTACCACAGTGGCTCACCAGGAAGGAAAATGAACAGGCACATGGATCTGTCTCCTGTCTGGTGAC |
| | | TCACTGTTCAAGTCCTAGCTATGGAACCTGAACAAGTCACTAAGTGTCTGTATTTGTTCTGTCTTGTCTGTCTCTGTCCC |
| | | CTTTGCACCGTGTGTGTGTGTGTGTGTGTGTGTGTGT |
| Phase-1 RCT-121 | | TATGACATGATTACGAATTTNATACGACTCACTATAGGGAATTTGGCCCTCGAGGCCAAGAATTCGGCACGAGGGATCC |
| | | AGACTGCAGCCTCCCTCCTCCCTCTCTCTAAGCCCTACCTCTGCACCTCCTCTTCCCCAGGTCCATTGTTCCTCTGTTT |
| | | CCCTTAAAAAAAAAAAAGAGCAGGCCTCCCAGGGATATCAGCTGAACACAGCATAACAAGATGCAATAAGAACAGGCA |
| | | CAAACCCTCTTATCAAGTCTGGATGAGGCAACCCAGTAGGAGGAAAAGGATCCCATAAGCAGGCAATAGTTGATATCA |
| | | ACATAGTTCCCAAAATAGAGATCTACACTGAGTTATTCTGGTTATGAGATGGAAAATGAGTGCAGTGCGCATGTGCTTC |
| | | TTGTTTGAGAACTTCCTTCTTTTTCCTCCCAAGTTCCTGGCTCCCAGCCACCAGCTGCCAGCACAACTCTCTCCACAGTT |
| | | AGTTCTTAGAATTTAGTAACAGATACTGAGATCTGTCTTTAGTAACAAATATAAAATGAGAATATAAGTCTAGAAATGATT |
| | | TTCCTGGGTTTTTCCGCTTTTGCAGTATCATGTTTATGGAATTTGAATTCTTGCAGCCATTGTATATGAATAGTCTGGGGT |
| | | AGAAGATGATCTGGAGACAAATTTTTTAGAAATAACTATTAAAGCTTGCTTTGATGCTGGAAAAAAAAA |
| Phase-1 RCT-123 | | TTATGACATGATTACGAATTTGATACGACTCACTATAGGGAATTTGGCCCTCGAGGCCAAGAATTCGGCACGAGGGTCC |
| | | CTCTGTCAGAGTCGGTGGGAGACAGAAGGGTTGAGAGAGGAGAACACTGCAGCTTAAGGTGTAAACGAGTATCGCAT |
| | | TTTATTTTCTAATAAATTCTCTAATAAGAAAAAAAAAAAAAAAAAACACATGCGGCCGCAAGCTTATTCCCTTTAGTGAGG |
| | | GTTAATTTTAGCTTGGCACTGGCCGTCGTTTTACAACGTCGTGACTGGGAAAACCCTGGCGTTACCCAACTTAATCGCC |
| | | TTGCAGCACATCCCCCTTTCGCCAGCTGGCGTAATAGCGAAGAGGCCCGCACCGATCGCCCTTCCCAACAGTTGCGC |
| | | AGCCTGAATGGCGAATGGGACGCGCCCTGTAGCGGCGCATTAAGCGCGGCGGGTGTGGTGGTTACGCGCAGCGTGA |
| | | CCGCTACACTTGCCAGCGCCCTAGCGCCCGCTCCTTTCGCTTTCTTCCTTCCTTTCTCGCCACGTTCGCCGGCTTTCCC |
| | | CGTCAAGCTCTAAATCGGGGGCTCCCTTTAGGGTTCCGATTTAGTGCTTTACGGCACCTCGACCCCAAAAAAACTTTGA |
| | | TTANGGTGATGGTTCACGTAATGGGCCATCGCCCTGATANACNGGTTTTTCNCCCTTTGACGTTGGAGTCCACCGTN |
| Phase-1 RCT-127 | | NTNNNNTCTATGACATGATTACGAATTTAATACGACTCACTATAGGGAATTTGGCCCTCGAGGCCAAGAATTCGGCACG |
| | | AGGCTGTTTGTGGTGTAGGGACAATGGCGCCATTGTTCTTCGCGTCTTCAAAGCAGTTTCCAGAGGAAACAAAGATGG |
| | | CGGGGAGCTCAAAGCATCCGAGAGAATTCGTGAAGGGAGGAAAGTTGCTTAAAACTTGACCATGTTAAAGTTCTTACTC |
| | | CTGGCACGTCATGATACTTTCTGCCAGCAGTGAATGCCAGCCCATTGAGATGCCCGTCCTCTGGGAAGCTCCTGGAGC |
| | | AGAAATGCATGGCCAGTGAGAGGAGACTTCTTGCGAAGGAAAACTGGATGTGGTGATGTTCAGAAAAAGCAGACACAA |
| | | GAAGCAAATACAGAGTATTAGTACTGGGATGTTTGAGATGGGGTCTGCATGGCCCTTGCTGTGTCCCTCTTAAACTGTC |
| | | TAGTGGATGGGACAGCATCTGGTATGGCCTGCAGAGTTGAGCCTTGTGTATACCTTTTGAAGGCTCTATGTTAATGTAT |
| | | GACAGAGTGTTCTATTCTGGGAAGGTTATGGGTGAGCTGAGGATACAACTTTACAGACTAGCACAATGAGTGTACAATT |
| | | ACTGTAAAAAAGGCAGTTCACATTTTTATTGGTTATGAATTGNTTCCTTATTCATCCAACAGTGATCACTTGGGAGTGCT |
| | | GCAATAAACT |
| Phase-1 RCT-128 | | TATGACATGATTACGAATTTAATACGACTCACTATAGGGAATTTGGCCCTCGAGGACAAGAATTCGGCACGAGGATACT |
| | | ATTAGACCAAGCAGCTCTCAAGTGTTTAATGTCTACTGTGACACCCAATCAGGCACTCCACGGACATTAATTCAACACC |
| | | GGAAAGATGGCTCTCAAAACTTCAACCAAACGTGGGAAAACTACGAAAAGGGTTTTGGGAGGCTTGATGGAGAATTCT |
| | | GGTTGGGCCTAGAGAAGATCTACGCTATAGTCAAACAGTCTAACTACATCTTACGACTGGAGCTACAAGACTGGAAGG |
| | | ACAGCAAGCACTATGCTGAATATTCCTTTCATCTGGGCAATCATGAAACCAACTACACGCTACATGTGGCTGAGATTGC |
| | | TGCCAATATCCCTGAGGCCCTACCAGAACACAGAGACCTGATGTTTTCTACATGGGATCACAGAGCAAAGGGACAGCT |
| | | CTACTGTCCAGAAAGTTATTCAGGTGGCTGGTGGTTCAGTGACATGTGTGGAGAAAACAACCTAAATGGTAAATACAAC |
| | | AAACCCAGAGCCAAATCCAAACCAGAGCGGAGAAGAGGAATCTCCTGGAGGCCTCGGGGCGGAAAGCTCTACTCTAT |
| | | CAAATCATCTAAAATGATGCTCCAGCAGACCACCTAAGGAACGTCAGCTGAACTGAGACAAATTAAAAGACCAACACAT |
| | | TCAATATTAAAATCC |
| Phase-1 RCT-137 | | TTATGACATGATTACGAATTTAATACGACTCACTATAGGGAATTTGGCCCTCGAGGCCAAGAATTCGGCACGAGGGTGA |
| | | TCTCGGTTCGTCGCCACCATGGGGAAGCGACAGCACCAGAAGGACAAGATGTACATCACCTGTGCAGAGTACACTCAT |
| | | TTCTATGGTGGCAGGAAGCCAGATATCACACAGACAAGTTTTCGCCGCTTACCTTTTGACCATTGCAGTCTCTCTCTCC |
| | | AGCCTTTTGTCTACCCAGTCTGCACCCCGGAAGGTGTCGTCTTCGACTTGCTGAACATTGTCCCATGGCTTAAGAAGTA |
| | | TGGGACCGATCCGAGCACTGGAGAGAAACTTGATGGGAAGTCCCTGATTAAGCTGAACTTTGCAAAGAACAGTGAAGG |
| | | GCAGTACCACTGTCCAGTGCTGTACTCCGTGTTCACTGACAACACCCACATCGTGGCCATCAGGACAACTGGCAATGT |
| | | CTACACCTATGAGGCAGTGGAGCAGCTAAACATCAAGGCCAAGAACTTGAGGGACCTGTTAACTGATGAGCCCTTTTC |
| | | CAGGCAAGACATCATCACCCTGCAGGACCCCACCAATCTGGACAAATTTTAACGTCAGCAGCTTCTTCCATGTAAAGAA |
| | | TAACATGCGAATGATAGATCCAGATGAGGAAAAGGCCAAACCAGACCCATCTTTNTATTTG |
| Phase-1 RCT-138 | | TCTATGACATGATTACGAATTTAATACGACTCACTATAGGGAATTTGGCCCTCGAGGCCAAGAATTCGGCACGAGGGTC |
| | | TGGCCACCATGGGGGCCCCAGAGCCCTCCTGGTGCTTTCTGTTCCTTCCTGTCCTCCTGACTGTGGGAGGATTAAGTC |
| | | CCGTACAGGCCCAGAGTGGTAAGCCATAACACCCCTGGTCTTTCTCTCTTCCTCTCAAGATTTCCTCAGGCTACCCCTT |
| | | TTCCTTCTAGCTCTCTCTCTCTGCTAACGCCGAGCCCTGATTGTTAACCTGTGTCTCCCTCTTCATCCTTCTGAGACAAT |
| | | TACCCAGGATGCGAATGTTCCTCTGTGAGCCCGGGTGTACTGGCTGGGATTGTGCTGGGCGACTTGGTGCTGACTCT |
| | | GCTCATCGCCCTGGCGGTGTACTCTCTGGGCCGCCTGGTCTCTCGAGGCCGAGGGACTGCAGACGGGACCCGGAAA |
| | | CAGCACATGGCTGAGACTGAGTCACCTTATCAGGAGCTTCAGGGTCAGAGGCCAGAAGTATACAGTGACCTCAACACA |
| | | CAGAGGCAGTATTACAGATGAACCCACCCTATGCCCACCAACAACCTGATGCCCGGATCCACTCATTCCAGACGCTTA |
| | | CTCAACAAACCCTCCCTGGGATCAGGACTCCCGCTGGAAATAAATATCCACAGAATGGCCTCTGGGAGATAT |
| Phase-1 RCT-139 | | GNGCTAAGNNTATGACATGATTACGAATTTAATACGACTCACTATANNGAATTTGGCCCTCGAGGCCAAGAATTCGGCA |
| | | CGAGGAAAGGTTTTTTTTTTTTTTTTTTTAAATGATTCCTTAAACAGGCCATCTGAGTAGGAACTGTAGGTTTATAAAATA |
| | | CATGGNAACTGAAGTTTCTGAGATGGGACTTGCTCTGTTAGCCAGGCTGATGTGGCTGGTGCTTGTCCCANCGCTAGG |
| | | GAGGAGAGAGATGTGTANATTTCCTAGGACATGTGGCCAGCCAGCCAGCCTGCTCAGGGACCCACCTGCCTCCATCG |
| | | TCCCAGGCGCTGATTACCCATATGCATGGTAGGTTCTAAAATTGAATTCAGGCCCTCAGGCTTGCAAGTTAAACATGAC |
| | | ATGAGCAGTCTCCCTGGCCCACTGGTTTCATGTGATCCCTAAANGCATTTCCCTTTGGCATANTAATCATGTTTTGACAA |
| | | TGCTACCATCCTTTGGGTATAAACTTACATGGTTTCCCCGGATATTTTGTTTAAAATGTGAATATTCTAAAAATGTTTAGA |
| | | GACTCNTTCNTACAATTTTCTATGATCAAAATATTTTNCTAAGTAGAAAATAAACTTTCNGANTAAAACTCTGTAACCCTG |
| | | GGGGNNNNNTNNNCNNNCCCNNNNNCNNNANNNNNNNNNNNNNNNNTNNNNNNNNNCCCCTTTGGNCCCCAAGGNT |
| | | TTTTNCNTTT |
| Phase-1 RCT-144 | | TTATNANATGATTACGAATTTAATACGACTCACTATAGGGAATTTGGCCCTCGAGGCCAAGAATTCGGCACGAGGGATT |
| | | CCCCCTTCAGGAGGTAAATGAAAAACCTAAGAAGAAAAAGCTAAAACCCCAGGAAACCCTACAGGAAAATGGAATGGA |
| | | AGACCCACCTGTCTCTTTGCCTAAAACCAAGAAAAAGAAAGCTTTTCCCAAGGAGGAGTTGGCCAGTGATCTTGAAGAG |
| | | ATGGCTACTAGCAGCATAAGTGTTCCTAAGAAAAAGAAGTCCTTACCTAAAGAGGAAGTGGCCAGTGAACCTGAAGAG |
| | | GCAGCAAGCCCCATCACCCCTAAGAAGAAAAGGAAATTTTCTGAGGAGCCTGAGGCTGCTGCAAGCTGCACAAAGAG |
| | | CAGCACAAAGAAAAAGAAAAAGTCGCAGAAGGCCCGGGAGGAGGATTAGAATGGACCTGCTTGGTGGGAGGGGCATA |
| | | CTTTATGGTGGCAGTTCCTCCTGCCCATGATAAACCCCAATAAAAACGCAAAACCCGAAAANNNANANNNNNAANNNNN |
| | | NNNNNNNAAAANAAAAAAAANNNTTCTCGCGGCCGCAAGCTTATTCCCTTTAGTGAGGGTTAATTTTAGCTTGGCA |
| | | CTGGNCGTCGTTTTACAACGTCGTGACTGGGAAAACCCTGGCGTTCCCAACTTTATTCGCCTTGCAGCACTTCCCCTTT |
| | | CGCCAGC |
| Phase-1 RCT-145 | | TATGACATGATTACGAATTTAATACGACTCACTATAGGGAATTTGGCCCTCGAGGCCAAGAATTCGGCACGAGGGCCG |
| | | AACCTCGCGGGGCGGGAAGCCGCGAGATGGACACCCCTCCGCTCTCAGACTCGGACTCCGGGTCGGATGAGTGCCT |
| | | GGCCTCAGATCAAGAGTTGCAGGATGCGTTTTCCCGCGGACTCCTAAAGCCAGGCCTCAATGTCGTGCTAGAGAAGC |
| | | CGAAGAAGGCGGTGAATGACGAGAATGGCCTGAAGCAGTGCTTGGCTGAATTCAAACGGGATCTGGAGTGGGTTGAA |
| | | AGGCTCGATGTGACCCTGGGTCCTGTGCCTGAAGCCAGTGAAACTCAGTCAACACCCCAGAACAAGGACCAGAAGAA |
| | | AGGTGTTAATCCAGAAGACGACTTCCAGAGGGAAATGAGTTTCTACCGCCAGGCCCAGGCTGCTGTGCTTGCAGTATT |
| | | ACCCCGACTCCACCAGCTCCAAGTCCCTACGAAGAGGCCCACTGATTATTTTGCAGAAATGGCCAAGTCTGATCAACA |
| | | GATGCAAAAGATTCGACAGAAGCTGCAGACTAAACAGGCTGCCATGGAGAAATCTGAAAAGGCCAAGCAACTTCGAGC |
| | | GCTTAGGAAATACGGAAAGAAGGTGCAAACTGAGGTCCTTCAGAAGAAGCAGCAGGAGAAAGCGCATATGA |
| Phase-1 RCT-146 | | GGGGGGGTCTCNANATGATTCGAATTTAATACGACTCACTATAGGGAATTTGGCCCTCGAGGCCAAGAATTCGGCACG |
| | | AGGGACATCTGTAGCTGGGGAGTCAGTTAANGTGGTCTCTTCCTGCGCGAACATGGTTCGGACCAAAGCAAACTACGT |
| | | CCNGGGAGCCTACAGNNNAGTGGTGGCTTCTCAAGCCCCTAGGAAGGTGCTTGGCTCCTCCACCTTTGTCACCAATTC |
| | | TTCCGGTTCGTCNAGAAAAGCTGAAAATAAGTATGCTGGAGGGAACCCAGTCTGTGTGCGCCCAACTCCCNANTGGCA |
| | | AAAAGGNATCGGNGAATTCTTCAAGCTGNCCCCTCANGATTCTAANGGATAAAACCNGATTCNTGTANANGCCCGNGG |
| | | CTGNGGCNTTTNAAAATNCNATANTNGATNAC |
| Phase-1 RCT-149 | | CTCTATGACATGATTACGAATTTAATACGACTCACTATAGGGAATTTGGCCCTCGAGGCCAAGAATTCGGCACGAGGG |
| | | GCGCGCCTACTCGGACATGAGAAAGGCCAACTGGAAAACTCAGACAAATACTTCCATGCTCGGGGGAACTATGATGC |
| | | CGCCCGAAGGGGTCCTGGGGGAGCCTGGGCTGCTAAAGTCATCAGTGATGCCAGAGAGGGTATTCAGAGATTAACAG |
| | | GACACGGAGCAGAGGACTCAAGAGCTGACCAGTTTGCCAACAAGTGGGGCCGGAGTGGCAAAGACCCCAACCACTTT |
| | | CGACCTGCTGGTCTGCCCAGGAAATACTGAATTTTCTCTTCATGTTGTTCCCGGGCGCACAGCCCCCCAAGGAAAGGG |
| | | GCAATTACTGAGTTGAGTTATTTCCTAAAACCTGGATCCCTAAACATCCCAATGTGCTGAATAAATGCTTGTGAAATGCA |
| | | NNANANNNAAAAAAAAANNAAANAAAAAAAAAAANAAAAACAAAAAAAAAAGTGAGCGGCCGCAAGCTTATTCCCTTTA |
| | | GTGAGGGTTAATTTTAGCTTGGCACTGGCCGTCGTTTTACAACGTCGTGACTGGGAAAACCCTGGCGTTACCCAACTTA |
| | | ATCGCCTTGCAGCANATCCCCCTTTTGCCAGCTGGCGTAATACCGAANAGGCCCGCCCCGATCGCCCTTCCCACAGTT |
| | | G |
| Phase-1 RCT-15 | | AGGTCTTATGANNTGATTACGAATTTAATACGACTCACTATATGGAATTGGCCCTCGAGGCCAAGAATTCGGCACGAG |
| | | GGGACACTTGCTTGAGTTTGGTGTTTTCCCCGCCATCCTGAGTTTCCCACTTGACTACCCTTTGAGCCCTCCGAAAAT |
| | | GAGATTCACCTGTGAGATGTTCCATCCCAACATCTACCCTGACGGAAGAGTGTGCATCTCCATCCTGCATGCCCCAGG |
| | | GGACGACCCCATGGGCTATGAGAGCAGTGCCGAGCGGTGGAGCCCAGTGCAGAGTGTGGAGAAGATCCTGCTTTCG |
| | | GTGGTGAGCATGCTGGCAGAGCCCAATGATGAGAGTGGAGCAAACGTAGACGCTTCCAAGATGTGGCGGGACGACC |
| | | GTGAGCAGTTCTACAAGATTGCCAAGCAGATCGTACAGAAGTCTCTGGGGCTCTGANGCTACAGCCGTGTGCACACTG |
| | | AGCAGTCCAGTGCTGTCCTGAAGACCACGTGGCGACATGCTGCCATGCCAACACCAACACAGGCAGCTGCAGAGCCT |
| | | CGGCCCCCATTGTTTCTTCCTTTNCACCCCAGACAGGCAGCTCCCCTGAGATCATGACTGGGATAATGACAGCTACTG |
| | | CACTGCAAAANAATCCTCCTGCTGCGCCTCAGGGANGGTCCAGGCTGTCCTCACTACCTCTCCAANCATGGGGCCTCA |
| | | NTCTGCTTCTTCTGANGACATGGG |
| Phase-1 RCT-152 | | TCCNAAGTGATTNTGCCNANAATACGACTCACTATAGGGAATTTGGCCCTCGAGGCCAAGAATTCGGCACGAGGATGG |
| | | GTTTCGGAGACCTGAAAACCCCCGCCGGCCTCCAGGTGCTCAACGATTACCTGGCGGACAAGAGCTACATTGAGGGG |
| | | TACGTGCCATCACAAGCCGATGTGGCAGTATTTGAAGCAATCTCTGGTCCACCACCCGCTGACCTGTGTCATGCTCTG |
| | | CGTTGGTATAATCATATCAAATCTTACGAAAAGAAAAGGCCAGCTTGCCGGGAGTGAAGAAATCTTTGGGCAAGTATG |
| | | GCCCTGTCAGTGTGGCAGATACCACAGGAAGTGGAGCAGCAGATGCTAAAGACGATGATGACATTGATCTCTTCGGAT |
| | | CTGATGATGAGGAGGAAAGTGAAGACGCAAAGAGGCTACGAGAAGAACGCCTTGCTCAGTATGAGTCAAAGAAAGCT |
| | | AAAAAGCCTGCAGTTGTTGCGAAGTCTTCCATCTTGTTAGATGTGAAGCCTTGGGACGATGAGACAGACATGACGAAA |
| | | CTTGAGGAGTGTGTCCGAAGCATTCAAGCGGACGGCTTGGTGTGGGGCTCCTCTAAATTGGTTCCAGTGGGATACGG |
| | | AATTAAAAAGCTTCAAATACAGTGTGTAGTTTGAAGATGATAAGGTTGGAACAGATATGCTGGAAANANCANATTACTGC |
| Phase-1 RCT-154 | | ATGACATGATTACGAATTTAATACGACTCACTATAGGGAATTTGGCCCTCGAGGCCAAGAATTCGGCACGAGGGAGAG |
| | | AAGCTGCCTTCTCATTGGCTGAGGCCAAATTCACAGCAGGGGACTTCAGCACCACAGTTATACAAAATGTAAATAAAGC |
| | | CCAGGTGAAGATCCGAGCAAAGAAAGACAATGTAGCAGGTGTCACCTTACCAGTGTTTGAACATTACCACGAAGGAAC |
| | | TGACAGCTATGAACTAACCGGTTTAGCCAGAGGTGGGGAGCAGCTGGCGAAACTTAAGCGGAATTATGCCAAAGCAGT |
| | | GGAACTACTGGTGGAACTGGCTTCCCTGCAGACTTCCTTTGTTACTCTGGATGAAGCCATTAAGATAACCAACAGGCGT |
| | | GTAAATGCTATTGAGCATGTCATCATTCCCCGGATTGAACGCACCCTTGCTTATATCATCACAGAACTGGATGAGAGAG |
| | | AGCGAGAAGAATTCTATAGGTTAAAGAAAATCCAGGAGAAGAAAAAGATTATCAAGGAGAAAAGCGAGAAGGACTTGG |
| | | AGCGGCGGAGAGCAGCTGGAGAAGTGATGGAGCCCGCCACCTCCTGGCGGAAAANAAGGACGANGACCTGCTGTTC |
| | | GAGTACTGCCCCTGCTCCCTTCAGGCTGAGCATCATTTAACTCATGGGCAGGNTCGGTATGTGCAGGTATT |
| Phase-1 RCT-162 | | ATGACATGATTACGAATTTAATACGACTCACTATAGGGAATTTGGCCCTCGAGGCCAAGAATTCGGCACGAGGAATTTA |
| | | AGCATATTAGTCAGCGGAGAAGCTTCGGCGAGCAGAAGTGGACTTGGAGCGCGTGCGGGTGTGGTACAAGCTGGAC |
| | | GAGCTGTTTGAGCAGGAACGTAATGTTCGCACAGCCATGACCAACAGAGCAGGATTGCTTGCCCTGATGCTGCACCAA |
| | | ACCATCCAACACGATCCACTTACTACCGACCTTCGCTCTAGTGCTGACCGCTGAAAGTCACCAGCCCAGAGCCTCTCA |
| | | GCCCTGCATTCAGTCAGGGAGGGGCTCTGCATTTCAGCTCGCTCTTCCTCCGTTCATCTGTTTATTCTACCACCCTTAG |
| | | TTTTCTTCTTACCATCCATGTTTTGGCTTCTGTTTGCCCTTATCAGAAGGGTCTCTGCTTTCCCTTTGTCTCCTCTCCATA |
| | | GTCAGTGCTGGGTGAAAGTCAAGTTTACTCAGCCTTGCCTATACCCTCCCCCAAAATAAACAGGTTTTGTTAATAAAATT |
| | | TTGAACAAGAATAAAAAAAAAAAAAAAAAAAAAAACAATTGCGGCCGCAAGCTTATTCCCTTTAGNGANGGTTAATTTTA |
| | | CTTGGCACTGGCCGTCGTTTTACAACGTCGTGGACTGGGAAAAACCTGGCGGTTACCCAACTTAATCGCCTTGC |
| Phase-1 RCT-164 | | NNCNAATCTATGACATGATTACGAATTTAATACGACTCACTATAGGGAATTTGGCCCTCGAGGCCAAGAATTCGGCACG |
| | | AGGATCTTACTGCCCAGTTGTTCTGGCATTCGAAAAAGGACTGTAGACTATGGTCTAATGTTCAAGGATGTGGATGGA |
| | | CAGGACTGTGGAAAATAGTGAGAAACTGGTTCTCCGCTGGAGGAAGTAGGGTTAGGTTTAGGACCTTTGCAAGTGGG |
| | | GGTCAGGCACACCACCAGGAACAGCTCCTTCGATAAATAAAACAGTTATCACATTCCCACAACAACCTAAACACAGACT |
| | | ACCTCTTCCCTTACCAGATCTGCTAAGCTGTGAGGTTCTAAGAGGTCTGAGTGTGTTGTTTAAACTTTTGGAGATGGTTT |
| | | TTCATTATAACAATTCAAGCCAGACATTTGAAAGTGACTGTTCTTATAGTGTCCCCAAGTCTACTAACTGAGGGCCCACC |
| | | TTCTAGTTTCTAGTTGCACTTCTGAAATCCCATGTTTTGTTTGCTGGGACCTGAGTTTGGTGGCATTTTAAAATACAGATG |
| | | AGATCAATTTATCCGACTTCATGAGTNATCCTNCATTCTCTCGAGAAAANCTGATTTCNATGTNAATTAATTGTACTATG |
| | | ACC |
| Phase-1 RCT-168 | | TCNATGACATGATTACGAATTTAATACGACTCACTATAGGGAATTTGGCCCTCGAGGCCAAGAATTCGGCACGAGGTAG |
| | | AAATGGCCGTGGATTTGTCACACGGGTGACCTAAAAAGACAGTGGGGACCTTGCTTGGGTTCCTGGGGTGCCCACTG |
| | | TCCAGCCACTAGGACACTTGTAAGGAGCTCTGTGCTATGGCACCGGGCCCGGTCTTCACTGTATATTCAAGGATAGCA |
| | | CACATATATATTAACTCTATTTATTTTGCTGACCCTGACTGCTGCACTTGTCGGTGACGTTTCTCCAGGTTAGGCTGGGG |
| | | TGATACTCTGGGATGCTCTGCGTACAAAGATTATTTGAATGAACTGATCTCAGGCCTGTCCTGTGTGTGTTGCTTTGGG |
| | | GGTGGGAACTGCTTTGAAACCCTTGTTCAGATGTTTTTATAGGCTGAGAATGTCTTGCTGTGATGGATTAAATTCCTTTT |
| | | TGAAAACTTTNNANANAAAAAANAAAAANAAAAAAAAACACTTGCGGCCGCAAGCTTATTCCCTTTAGTGAGTCGTATTA |
| | | ATTTTAGCTTGGCACTGGCCGTCGTTTTACAACGTCGTGACTGGGAAAACCCTGGCGTTACCCAACTTAATCGCCTTGC |
| | | AGCACATCCCCCTTTCGCCAGCTGGCGTAATAGCGAAAAAGNCCGCACCGATCGCCCTTCCC |
| Phase-1 RCT-169 | | TATGACATGATTACGAATTTAATACGACTCACTATAGGGAATTTGGCCCTCGAGGCCAAGAATTCGGCACGAGGAGCAC |
| | | GGTGACTTAATAGAGCTCGGCTCTGCCATGAAGTCCGTTGCTCTATTCCTCATGGGCATCATCTTCCTGGATCACTGTG |
| | | GAGTTCGAGGAACCCTAGTGATAAGGAATCAGCGATGCTCCTGCATCAGCACCAGCCAAGGCACATTCCACTACAAAT |
| | | CCCTCAAAGACCTCAAACAGTTTGCCCCAAGCCCTAACTGCAACAAAACTGAAATCATCGCTACACTGAAGAACGGAGA |
| | | TCAAACCTGCCTAGACCCAGATTCAGCAAGGGTGAAGAAGCTGATGAAAGAATGGGAGAAAAAGATCAGCCAAAAGAA |
| | | AAAGCAAAAGAGGGGGAAAAACCATCAAAGGAGCAAGAAAACCCGAAAAGCTAAAACACCCCACCATCCGGAGTCAAA |
| | | GAAGACTGCATAAGAGACCACTTTACCAACAAGCGCTCTGCATCTAAACAGCTTTTAGATCATACTAAAACGCCTTCCCT |
| | | TTAATACACAACTCGTTCACTACAAAAAAAAAAAAAAAAAAGTGAGCGGCCGCAAGCTTATTCCCTTTAGTGAGGGTTAA |
| | | TTTTAGCTTGGCACTGGCCGTCGTTTTACAACGTCGTGGACTGGGAAAACCCTGGCGTTACCCAACTTAATCGCCTTGC |
| | | AGCACATCCCCTTTCGC |
| Phase-1 RCT-173 | | TATGACATGATTACGAATTTAATACGACTCACTATAGGGAATTTGGCCCTCGAGGCCAAGAATTCGGCACGAGGCAGAC |
| | | TCTGGAGAAGGTGTGCGTGCAGACTGTGGAGAGCGGAGCTATGACCAAGGACCTGGCTGGCTGCATCCATGGCCTCA |
| | | GCAATGTGAAGCTGAATGAGCACTTCCTGAACACCACAGACTTCCTGGACACCATTAAGAGCAACCTGGACAGAGCTC |
| | | TGGGCAAGCAGTAGGGGTGGACACTACACTACCCAGCTCCAGTGGGTGCTCCGTACATGGTAGAGGGTGGGCGTTGT |
| | | AGCCCCCCTCTCTGGTGGCCTTTCTAGGGGATTATTTTTTTACATATATATATATATGATGGAGATGTTTTTTGCATG |
| | | TGAGCAATTTCCCTCACAGTGGACACGAGGCAGGAACGGTGCGCTTTACCTCAGCCAGTCAGTGTGTTTTGCATACTG |
| | | TAATTTATATTGCCCTGAGTCACGTGGTGCCATATTTAGCTACTAAAAAGCTCTTCACAAANNNNCNNNNNNNNNNNAAT |
| | | NNNNNCAATTGCGGCCGCAAGCTTATTCCCTTTAGTGAGGGTTAATTTTAGCTTGGCACTGGCCGTCGTTTTACAACGT |
| | | CGTGACTGGNAAAACCCTGGCGTTACCCAACTTTAATCGCCTTGNAGCACATCCCCCTTTCGCCAGCTGGCGTAA |
| Phase-1 RCT-177 | | CCNNNNNNNCTCTATGACATGATTACGAATTTAATACGACTCACTATAGGGAAGGCCCTCGAGGCCAAGAATTCGG |
| | | CACGAGGGAAGCCTCGGGGAGGAGAGACAGCTCTGTTCCGTTTTTGTTTCTGGTGAAGAGTGTGGGGATTTTCGTCTT |
| | | GGCTTCAAATCCGTCCAAGCAGCACAGCGGGTCAAAATCTCCTGCAGGTCCTCAGATTACCAGTGACTAAACTGCACC |
| | | TAGGCAGACCAGCCATGAGAGCCACTCAGCAGGACTTCGAAAATGCAATGAACCAGGTGAAACTCTTGAAGAAGGACC |
| | | CAGGAAATGAAGTGAAGCTGAGACTGTATGCGCTGTATAAGCAGGCCACAGAAGGACCCTGCACTATGCCTAAACCAG |
| | | GTGTGTTTGACTTTGTCAATAAAGCCAAGTGGGATGCATGGAATGCTCTGGGCAGCCTACCAAAGGAAACTGCCAGGC |
| | | AGAACTATGTGGACCTCGTATCCAGTTTGAGTTCCTCATCTGAAGCCTCGAGCCAGGGAAAGGGTGGAGCTGATGGGA |
| | | AAGCCCAGGAGTCCAAGGGCATNCTGGTAACGTCTGAANGTGGCATCACAAAGATCACGTTCAATCGGCCTCCAAAAA |
| | | GAACGCCATAACCTTCCAGATGTATCAGGATATTATACTCGCGCTTAAGAATGCCAGCACGGATGACACAGTNCATCAC |
| | | CGTTTTCACANGAGCTGGTGACT |
| Phase-1 RCT-179 | | TCCTCATAANATGATTACNAATTTAATACGACTCACTATAGGGAAGGCCCTCGAGGCCAAGAATTCGGCACGAGGG |
| | | TTCTGCGGAACAGTAGGCAGTTGTTTTCCGTCCGGCTTCTCTCACACTCAAGTGCGCGCCTCCACCTCATGGAAGACT |
| | | CGATGGACATGGACATGAGCCCTCTTAGGCCTCAGAACTACCTTTTCGGTTGTGAACTAAAGGCTGACAAAGATTATCA |
| | | CTTTAAAGTGGATAATGATGAAAATGAGCACCAGTTATCATTAAGAACGGTCAGTTTAGGAGCAGGGGCAAAAGATGAG |
| | | TTGCACATCGTAGAGGCAGAAGCAATGAACTATGAAGGCAGCCCAATTAAAGTAACACTGGCAACTTTGAAAATGTCTG |
| | | TACAACCAACAGTTTCCCTTGGGGGCTTCGAAATTACACCACCTGTGGTCTTGAGGTTGAAGNGTGGTTCTGGGCCTG |
| | | TGCACATAAGTGGAXCAGCACCTAGTAGCTGTAGAGGAAGATGCANAGTCANAAGATGAAGATGAGGAAGATGTNAAAC |
| | | TCTTAGCATGTCTGGAAANAGATCTGCTCCCGGAGGTGGTAACAAAGTCCCACAGAAAAAATAAAACTTGATGAANAT |
| | | GATGATGANGATGATGAAGATGATGAGGATGAATGAANATGATGATGATGATGATTTTGATC |
| Phase-1 RCT-18 | | TTATGACATGATTACGAATTTAATACGACTCACTATAGGGAATTTGGCCCTCGAGGCCAAGAATTCGGCACGAGGAAGT |
| | | TATACGCCCTGGGAATGGCTGCCCCAAAACTGAAATCATTTTCTGGACCAAGGCCAAGAAAGCTATATGTGTGAATCCT |
| | | ACTGCCAGATGGTTACCAAAAGTATTAAAATTTGTCCGAAGCAGAAGTATTACTTCAACTCCCCAAGCTCCAGTGAGTA |
| | | AGAAAAGAGCTGCCTGAAGCCACTGTCACCCCAAAAGAACCTGCACCCTTTCTTAATCCCTGCTCGGAATCTTAGTGTT |
| | | ACGTTCTTAGTTGAAGAATTTCCAAGAAAATAACTTCCCTCTACAAACACGGCTGTAGATTAAAAGAAAAAAATCCTGCA |
| | | GTAGCTGAGAGGAGACACTCGAGCTCCTTCCCATACTCAACCCATATTCTTGTTCCTTAAGGGAGGATATTTTCGAGCA |
| | | GGCATTTAGTGACAAGCCACTTTGGTAATAGACCTGTTGTTTAGTGTTAACTATCCTAGACCTAGAGGAATAAAAGCAT |
| | | ACATGTCGAATCTGAACCATAGCTCCTACTAACAAGAGGTTTATGAGATGGACTTCAGTTAGTTTGCACCCTTGCAAAAA |
| | | TCAGGCTTCCAGAATAGTTTCCAGAAAGTCCCTAAGAAGCAGACGCATTACCAGCCTAAGGNGANGCAGAGCAGGTCT |
| | | CCNTTAGAGAGAATCTTCTGGAGGGAAATAATGNTTN |
| Phase-1 RCT-180 | | TCTATGACATGATTACGAATTTAATAGGACTCACTATAGGGAATTTGGCCCTCGAGGCCAAGAATTCGGCACGAGGGTG |
| | | GTGGCCAAGTTCAACGCCTCGCAGCTCATTACCCAGCGGGCTCAGGTGTCTCTGTTGATCCGAAGAGAGCTGACAGA |
| | | GCGTGCGAAGGACTTCAGCCTCATCCTGGACGATGTAGCTATCACAGAGCTAAGCTTCAGCCGAGAGTACACAGCTGC |
| | | TGTAGAAGCCAAACAAGTGGCCCAGCAGGAAGCCCAGAGGGCCCAGTTTTTGGTGGAGAAAGCAAAGCAGGAACAAC |
| | | GACAGAAGATTGTGCAGGCTGAGGGGGAGGCGGAGGCTGCTAAGATGCTTGGAGAAGCACTGAGCAAGAATCCTGG |
| | | CTATATCAAGCTCCGAAAGATCCGGGCTGCCCAGAACATCTCTAAAACGATCGCCACATCACAGAACCGGATCTATCTC |
| | | ACAGCTGACAACCTTGTGCTGAATCTGCAGGATGAAAGCTTTACTCGGGGAAGTGACAGCCTCATTAAGGGTAAGAAG |
| | | TGAGTGTGGACATCAAGAACCCCCACCACCAGAGAAGTTGGCACACTTCTCCAGTTTGGAGGGGCCAGCTTAGGGGG |
| | | TCAAGCATACCCCANCCCTGACCCAAGCATCATGNGATGGATTCTTCTGTATCTGCTCTCTTGGGATTAANGGAAACTG |
| | | AAGAC |
| Phase-1 RCT-181 | | TATGACATGATTACGAATTTAATACGACTCACTATAGGGAATTTGGCCCTCGAGGCCAAGAATTCGGCACGAGGCTGAA |
| | | CATCTACGTGCCACCGTGCGCTGGGATTACCAGCCAGACATCTGTAAGGACTACAAGGAAACTGGCTTCTGTGGTTTT |
| | | GGAGACAGCTGCAAATTCCTCCATGACCGTTCAGATTAGAAGCACGGATGGCAGATAGAACGGGAGCTCGATGAGGG |
| | | CCGTTATGGTGTGTATGAAGATGAAAACTATGAAGTAGAAAGCGATGATGAGGAAATACCATTCAAATGTTTCATCTGTC |
| | | GCCAAACCTTCCAGAATCCGGTTGTTACTAAGTGTAAACATTATTTCTGCGAGACCTGTGCATTGCAGCATTTCCGAAC |
| | | CACTCCACGGTGCTATGTTTGTGAGCAACAGACCCATGGGGTTTTCAATCCTGCCAAAGAACTGATTGCTAAACTGGAG |
| | | AAATACCGAAAAGCGGAAGGTGGTACTTCTAACACTTCAGAAGACGCCGATGGAATCTAATTGCCTTTACTTAGATTTTT |
| | | TGCAATTGTCAAATCTCAAAGTACTTTTTGTTTGGTCACTTCAAACCAAAGCAACCGTAATTGAAGAAATATTAAGTTTT |
| | | CTAAGAAGAATATCCAAGTTTTGTATTATATGACAGTGCTACAGATGGGAA |
| Phase-1 RCT-182 | | TATGACATGATTACGAATTTAATACGACTCACTATAGGGAATTTGGCCCTCGAGGCCAAGAATTCGGCACGAGGGTGAA |
| | | TGTCTCCAGCCAGGCCTCCCAGCGTGCACTGACCAACCATACCGTCTACTGTTCCACCAAGGGTGCTTTAGACATGTT |
| | | GACCAAGGTGATGGCCCTAGAGCTTGGGCCCCACAAGATCCGTGTGAATGCAGTAAACCCCACAGTAGTGATGACAC |
| | | CCATGGGCCGGGCCAACTGGAGTGACCCGCACAAAGCTAAGGTCATGCTGGATCGTATCCCACTTGGCAAGTTTGCT |
| | | GAGGTGGAGAACGTGGTANACACCATCCTCTTCCTGCTGAGCAACCGAAGTAGCATGACTACTGGTTCCGCTTTGCCA |
| | | GTGGATGGGGGCTTCCTGGCTACCTAAGCCCTCCCTACCAATACTCTGCTCAACTCATGTTCAGAACATCGTGCCCTC |
| | | CATCCCTCCAATAAAGCTCTCTGCCCAGCCTGTGTGCTGATTCTCCACCCCNNANAAAAAAAATTCACANNAANNANAA |
| | | NAAAAAAAGTTTGGCGGCCGCAAGCTTATTCCCTTTAGTGAGGGTTAAATTTTAGCTTGGCACTGGCCGNCGTTTTACA |
| | | ACGTCGTGACTGGGAAAACCTGGCGTTACCCCACTTAATCGCCTTGCAGCANATCCCCCTTTCGCC |
| Phase-1 RCT-185 | | NCTATGACATGATTACGAATTTAATACGACTCACTATAGGGAATTTGGCCCTCGAGGCCAAGAATTCGGCACGAGGGAA |
| | | TTAAGCTCCAGCAGACCAGCTGCCTGAAGAAGGACTGGAAAAAGCCGGAGTGTACAATCAAACCAAATGGGAGGAA |
| | | GCGGAAATGCCTGGCCTGCATCAAACTGGACCCCAAGGGTAAAGTTCTAGGCCGGATGGTCCACTGCCCAATACTGA |
| | | AGCAAGGGCCTCAGCAGGAGCCTCAGGAATCCCAGTGCAGTAAGATAGCACAGGCCGGCGAGGACTCCCGCATCTAC |
| | | TTCTTCCCTGGGCAGTTTGCCTTCTCAGGGCTCTACAATCCAAATAAGCCCTGGACAGGGTTTCATCTTACTTCCTGTA |
| | | CAGCCGTGGCGGTACCCACCATATGGCCTCCCAAAGACTTTCAACTCCAGGCTAATAAAACTGTTCCTTTCCAAAAAAA |
| | | AAAAAAAAAAAAAAAAAAGTGTGGCGGCCGCAAGCTTATTCCCTTTAGTGAGGGTTAATTTTAGCTTGGCACTGGCCGT |
| | | CGTTTTACAACGTCGTGACTGGGAAAACCCTGGCGTTACCCAACTTAATCGCCTTGCAGCACATCCCCCCTTTCGCCAG |
| | | CTGGGCGGNAATAANCGAAANAGGNCCCCCACCNNTN |
| Phase-1 RCT-187 | | TCTATGACATGATTACGAATTTAATACGACTCACTATAGGGAATTTGGCCCTCGAGGCCAAGAATTCGGCACGAGGAAA |
| | | AGCCGGGGGAGTATGGACCTCACTCTTCTGTGGGTGCTTCTGCCACTGGTGAGCACAGCCTGGGGCCAGTATGGTGA |
| | | CTACGGGTACCATCCATACCAGCAGTACCAGGACTACAGTGACGACGGATGGGTGAACCTGAACCGTCAGGGCTTCA |
| | | GCTACCAGTGTCCCCACGGGCAAGTGGTGGTAGCTGTGAGGAGCATCTTTAACAAGAAGGAGGGCTCTGACAGACAG |
| | | TGGAACTATGCCTGCATGCCCACTCCCCAAAGCCTCGGGGAGCCCACTGAGTGCTGGTGGGAGGAGATCAACAGGGC |
| | | CGGCATGGAATGGATGACAACCGAGTACCCAGGCCACTACGGCGAAGACATGGACATGATTTCCTATGACTATGATTT |
| | | CTATATGAGAGGAGCAACAACCACTTTCTCTGCAGTGGAAAGGGATCGCCAGTGGAAGTTCATAATGTGCCGGATGAC |
| | | TGACTATGACTGTGAAATTGAAAACGTTTAGATTCCCATGTACAGAAGTCCGGGTGAAGGGCAAGGGTCCTGAGGGTG |
| | | TCATATCACATCTGCTAATATTAATTAGAACAACTGCACTTTTT |
| Phase-1 RCT-189 | | GCCNGGGATATGCNCNGATTACNNGATTCACTGGCCGTCGTTTTACAACGTCGTGACTGGNAAANCCCTGGCGTTACC |
| | | CAANTNANTNGCCTTGNTGGCGACTGTGGAACCGGAAACCACCCCTACCACTAATCCCCCACCTGCGGAAGAGGAAAA |
| | | AACCTTCTGTTTTCTGTCACAGCATTGTCCTTGCTGTCCTCTTGCTGATGGCTGCTAGATGTTAATTTATTTGCTTCCCTC |
| | | CTTGATAACATTAGGTGATTCTGATTTCAANTTTTTCATTTGTTTTGCTTTTGTTTTTTTCCTCATGTAACATTGGTGAAGG |
| | | ATCCAGGACTATGACAGAAAGGTGGAATAAACATTACATTTGTGCATTCTTTGGGTAATTTTTTTGGTTTTTGTAACTACA |
| | | AAGCTTTGCTACAAATTTATGCATTTCATTCAAATCAGTGATCTATGTCTGTGTGATCCCTAAACATAATTGTGGACTATA |
| | | AAAATGTAACACCATAAATTACATTCCTAACTANAATTAGTTGNCTGCTTTTGAATCTTTATGCCTGNACTTTAACACTTT |
| | | GGGNTACTTAGGTTATTTTGNTTTGGGTTACAATGGCTCAAGTAGAAAAGCCGGTCCCATCCTTTTTAAAGACAGTGTA |
| | | CAAAACTGGNNATNNAATGTGNTCANGNGAATTGTCTTTTAAAAAAAAAAAAAAAAT |
| Phase-1 RCT-191 | | TATGACATGATTACGAATTTAATACGACTCACTATAGGGAATTTGGCCCTCGAGGCCAAGAATTCGGCACGAGGCTGAG |
| | | AAGCTAGTGTCCAAGTGTGTGTTCCCCGAACAGGCTAACAACAATGAGTGGGCCAGGTACCTCTACTACACAGGGCGA |
| | | ATCAAGGCTATCCAACTGGAGTACTCTGAAGCCCGGAGAACGATGACCAATGCCCTTCGAAAGGCACCACAGCACACA |
| | | GCAGTTGGCTTCAAACAGACGGTGCACAAGCTTCTGATTGTGGTGGAGCTGCTGCTGGGGGAGATCCCAGACCGCCT |
| | | GCAGTTCCGTCAGCCCTCCCTAAAGCGCTCTCTCATGCCCTACTTCCTTCTCACCCAAGCTGTTAGGACAGGAAATCTT |
| | | GCCAAGTTCAACCAAGTCCTGGATCAGTTTGGGGAGAAGTTTCAAACAGATGGGACCTACACCCTAATTATCCGACTG |
| | | CGACACAATGTGATTAAGACAGGTGTGCGTATGATCAGCCTGTCCTATTCCCGTATCTCCCTGGCGGACATCGCCCAG |
| | | AAGCTGCAGCTGGACAGCCCAGAAGATGCAGAGTTCATTGTCGCCAAGGCTATCCGGGATGGTGTGATTGANGCCAG |
| | | CATCAATCATGAGAAGGGATACGTCCAATCCAAAGANATGATTGACATCTACTCCACCCGGGAACTCAGCT |
| Phase-1 RCT-192 | | CTATGACATGATTACGAATTTAATACGACTCACTATAGGGAATTTGGCCCTCGAGGCCAAGAATTCGGCACGAGGGTTT |
| | | CCGCTTGCTGCTCCGCCATGGCTCGCGGTCCCAAGAAACACCTGAAGCGTGTGGCGGCCCCACGTCACTGGATGCTG |
| | | GACAAACTGACCGGCGTGTTCGGGCCCCGGCCATCTGCCGGCCCGCACCGCCTGCGGGAGTGCCTGGCGCTCGCCA |
| | | TCTTCCTGAGGAATAGGCTCAAGTACGCTGTGACCGGCGATGAGGTGAAGAAGATCTGCATGCAGCGCCTCATTAAGG |
| | | TCGACGGCAAGGTCAGAACCGATGTGGCCTACCCAGCTGGCTTCATGGATGTCATCAGCATAGACAAGAGCGGTGAG |
| | | AACTTCCGCCTGGTCTACGACACCAAGGGCCGCTTCGCGGTGCACCGCATCACGCCGGAGGAGGCCAAGTACAAGCT |
| | | GTGCAAGGTGAGGAAGGTCTTCGTGGGTACCAAGGGCATCCCGCACCTCGTGACGCACGACGCGCGAACCATCCGC |
| | | TACCCTGACCGCTCATCAAGGTCAACGACACCGTGCAGATCTCGCTAGACAGCGGCAAAATCACCGATGCCATCAAGT |
| | | TTGATACCGGCAACCTGTGCATGGTAACCCGGAAGGGGCCAACCTGGGCCGCATCGGC |
| Phase-1 RCT-197 | | TTCTATGACATGATTACGAATTTAATACGACTCACTATAGGGAATTTGGCCCTCGAGGCCAAGAATTCGGCACGAGGGG |
| | | AGGTGCCTTCTGTGTTCTAGCCTTGGTGATCTTAGCAACTTTAAGCCTGTCAACAATATCTGATTTTAAGAATGTAGCAG |
| | | TGTGGGAAGATGGTCATGGACCTTTAGATGTCTCAGGAACTGAAAGTTCAGAGACATCTAAACCACCACGTCTCACACC |
| | | ACCATGATCCTGATGAACTCAACGGCTGCGATGAACTCAACTGCTGCACCCATTCGTTCCCAGCAAATAGGAGAGAAA |
| | | TTAATTGCAGTTACTAATAACATGACTGTTCGAGAAAAGCCCCCCCCTTTGGGAAAGTTTTGTTTAGCATGATTCAGAAT |
| | | AGTAGTGACTCTTAGAAAGATCATGGATAAGTTCCAACAAGTTGAGCAAATTTATCAAGAGTTAACTAGAAGGAAAAGA |
| | | GAAACTAACATTGAGCAAGAAACGAAAGAAATTATAGAATGGTTAGAAAAGTTTTTATTCTGAGGGCCCATAGAG |
| | | TTTAAACTTTATTAAAATAAAGGTAAATGTTAAATGTATATCTGGGTACCCACAAGTCTGGTAGTATAACTGCAGNTTTCT |
| | | AAACTATTGTTTGCGGCTGAGAA |
| Phase-1 RCT-20 | | TNCTTATGACATGATTACGAATTTAATACGACTCACTATAGGGAATTTGGCCCTCGAGGCCAAGAATTCGGCACGAGGT |
| | | TTTCAAGGAAGGTAAGGTTTATTATTTTGGCTCACGGTTCCAGAGGATACAATCTATAATGGCTGAGGAGGCAGCAGTG |
| | | GAAGTATGGGACAGAGGTAATTCATACATCCTGACTGGATAAGACCAGCAGGAGTATATAATCCTCACCCATCGATATC |
| | | CTTAAGGTTTCACAGCCTTCAAAATAGAGCCTTAAGTTGAAAGACCAACAGCTCAAAACAAGCCTCCAGGTACATTTCA |
| | | GATTCAAGCGTCAACACACACCTTAATATATTTCAACGAAGCTACACATTCACAATTTAAATACAGTTTTTCATCAGGGAG |
| | | ACAGATATAATTCATAAGAGTGTTTAGACTACAAATCAGATACAATTTAGTCAAAATACCATAAAGAGTACTTTAAGTAAT |
| | | CAGGAAAGAGATTTTAACATTATTGCTTACTCTTATGAATTTTAACCAAATTGATAATTCTATAATCTTAGTTTATTTTCTAT |
| | | AGACTTAAATTATACTGCTTGCCAAGTAATGCAAGTATTGATGCAACTTTCTGAAATTAATTGAACTGCAAACCAAATTAT |
| | | TAAACTTAGCCAGCCACACACATTTTGACTCCCACTGCTTCAGAAGTAGTACCTATAAAAAGTAACCCACTGTAN |
| Phase-1 RCT-204 | | CTTTATGACATGATTACGAATTTAATACGACTCACTATAGGGAATTTGGCCCTCGAGGCCAAGAATTCGGCACGAGGCT |
| | | AGCTTTCGTTTCTTTGCTATGTCTGGACGTGGGAAGCAGGGCGGCAAGGCTCGCGCCAAGGCCAAGACCCGGTCCTC |
| | | CCGGGCGGGCCTGCAGTTTCCCGTGGGCCGCGTGCACCGTCTGCTGCGTAAGGGCAACTATTCGGAGCGGGTGGGC |
| | | GCCGGCGCCCCGGTGTACCTGGCGGCCGTGCTGGAGTACCTGACGGCCGAGATCCTGGAGCTGGCGGGCAACGCA |
| | | GCGAGGGACAACAAGAAGACGCGCATCATCCCGCGCCACCTGCAGCTGGCCATCCGCAACGACGAGGAGCTCAACA |
| | | AGCTGCTGGGCCGCGTGACCATCGCACAGGGCGGCGTTCTGCCAAACATCCAGGCGGTGCTGCTGCCCAAGAAGAC |
| | | CGAGAGCCACCACAAGGCTAAGGGGAAGTAAGACCAGGGATCACCAAAGGCTCTTTTCAGAGCCNCCTATAAAAAAAA |
| | | AAAAAAAAAAAAAAAAAACNATTGCGGCCGCAAGCTTATTCCCTTTAGTGAGGGTTAATTTTACTTGGCACTGGCCGTC |
| | | GTTTTACANCGTCGTGACTGGGAAAAACCCTGGNGTTACCCCAACTTAANNCCCTTGCAGCANNTCCCCCTTTCGC |
| Phase-1 RCT-205 | | CAACNNCNNNCNCNNTTATGNCATGATTACGAATTTAATACGACTCACTATAGGGAATTTGGCCCTCGAGGCCAAGAAT |
| | | TCGGCACGAGGTGTGTGTGTAACTGGATCAGCAGCATGTGTTCCAGTCATGGTTGACCTTAGCAGACATTAGCAGGAT |
| | | TAACACTGGAGGGAAACAAAGAAGCAAAATAAATATTTCCTCTGAGGTGGTATCTGCTGCTTGAACTGTTGTGCATTCA |
| | | TTGAACTTTGGACTTCAGAGGTTGCCTTATTGCATCTGAAATTCTTGGTCTAAAGTGAGATCCGAATTTCTTTTGCGCAC |
| | | CTTCACGAAAAAGTATAGAAGCCATATAGTTGGAACTTTGTGTGGGTTATGTCTAAACCTAGGAATAAATGCTGGGTTTC |
| | | TTTTCCTGTTGTAATTTCAGTCAAGTTACTGTGAGGGAAGCTGTTAGGTACATTTTAAATAAATTACAGCAAAGCGCTAT |
| | | CCTTCTGATCACAGCCAAGCTAACATCTATCTTCCGAACATCATGTCTGCCGCCTGCTGAAAGGCTGTAANGGTCTGGG |
| | | TAGTTTTCATTTAATATTGATCAAAGAAGCGTTTATTTATAAGNGCAAAGTGTTTTTTGGACGTTAATAAAAAAAAAAGTG |
| | | TTGNCCAACTAAAAAAAAAAAAAAAAAAAAAAAAATGGAAGCGGGCCGCAAGCTTATTNCCTTTANTGANGGTTAATTTT |
| | | AACTTGGCACTGGCCGGCGNTTTACAACGTCNNGAATGGGGAAAACCTGGNGG |
| Phase-1 RCT-207 | | TTATGACATGATTACGAATTTAATACGACTCACTATAGGGAATTTGGCCCTCGAGGCCAAGAATTCGGCACGAGGTCAT |
| | | GTGCGATGCTCTCATCAAGGCCATTGGCACGGAGCCTGACTCAGACGTCTTCTCGGAAATAATGCACTCCTTTGCTAA |
| | | CNGCATTGAANNGANGGGAGATGGGNGTCTCAACAATGAACACTTCNAGGAACTGGGAGGTATACTGAAGGCGAAGC |
| | | TCGAGGAACATTTCAAAAATCAANAGTTGCGGCAAGTTAAAAGACAAGATGAAGACTACGACGAACAGGTTGAANAGTC |
| | | NCTACAAGATGAANATGATAATGATGTTTATATACTGACTAAAGTCTCANATATTTTACACTCAATATTCAGTANCTACAA |
| | | ANAAAAGGTGTTGCCGNGGTTTGAACAGCTGCTCCCATTAATTGTCAACCTGATTTGTCCACATAAACCCTGGCCANAC |
| | | ANACAATGGGGATTGNGCATCTTCGATGATATCATANANCACTGTAGTCCAGCTTCATTTAAGTATGCAGAATATTTCTT |
| | | AAGGCCAATGCTCCAGTATGTATGTGACAACANCCCGGAAGTCAGGCAAGCTGCAGCATATGGCCTTGGCGTCATGG |
| | | CNCAATACGGNGGANATAACTACCGCC |
| Phase-1 RCT-211 | | TATGACATGATTACGAATTTAATACGACTCACTATAGGGAATTTGGCCCTCGAGGCCAAGAATTCGGCACGAGGAAGAA |
| | | GCAGCGAGGACGACCCCCGGACGGACCAAACCCGTGCGCCGCTGTAAACCCGTGCTCAGCGCCGACGTCCCCTGCC |
| | | GCCGCCATGCCCAAAAGAAATGCTGAAGGGGATGCTAAAGGAGACAAAGCCAAGGTGAAGGACGAGGCACAGAGAA |
| | | GATCTGCAAGGTTGTCTGCTAAACCTGCTCCTCCAAAGCCAGAGCCCAAGCCTAAAAAGGCCCCTGCAAAGAAGGGAG |
| | | AGAAGGTACCCAAGGGGAAGAAGGGGAAAGCGGATGCTGGTAAGGATGCGAATAATCCTGCAGAAAACGGAGATGCC |
| | | AAAACAGACCAGGCACAGAAAGCGGACGGTGCTGGAGATGCCAAGTGACGTGTGTGCGTTTTTGATAACTGTGTACTT |
| | | CTGGTGACTGTACAGTTTGAAATACTATTTTTTATCAAGTTTTATANNNNAAAAAAAAAAAANAAAAAAAAAACAATTGCG |
| | | GCCGCAAGCTTATTCCCTTTAGTGAGGGTTAATTTTAGCTTGGCACTGGCCGTCGTTTTACAACGTCGTGACTGGGAAA |
| | | ACCCTGGCGTTACCCAACTTAATCGCCTTGCAGCACATCCCCCTTTCCCAGCTGGNGTAATAACGAANAAGNCCGCAC |
| | | CGATCGCCCTTCCAACAGTTTGCGC |
| Phase-1 RCT-213 | | TATGACATGATTACGAATTTAATACGACTCACTATAGGGAATTTGGCCCTCGAGGCCAAGAATTCGGCACGAGGATTGT |
| | | AGCATCTATGGAGAAGACACCGTGACAGACGAGGAGGGGAAGTTCCGGTTACGTGGACTGCTGCGAGGGTGCATGTA |
| | | CCACGTGCAGCTGAAGGCAGAAGGCAATGACCACATCGAGCGGGCGCTGCCCCACCATCGGGTGATTGAGGTTGGG |
| | | AACAATGACGTGGATGACGTAAACATCATAGTTTTCCGGCAGATCAACCAGTTTGACCTGAGTGGAAATGTAATCACTT |
| | | CCTCCGAGTATCTGTCTACTTTGTGGGTGAAGCTGTACAAGAGCGAGAGTCTCGACAACCCCATCCAGACAGTGTCTC |
| | | TGGGTCAGTCGCTCTTCTTCCATTTCCCACCGCTGCTCCGAGATGGTGAGAACTACGTGGTGCTTCTGGACTCCACGC |
| | | TCCCCAGGTCTCAGTACGACTACGTCCTGCCGCAAGTGTCTTTCGCTGCAGTGGGCTACCACAAACACATCACTCTGG |
| | | TGTTCAGTCCCACGAGGAACTNCCTGAGCAAGAGATCCGCAAGGGNCTATATTGCCCTGCCTCTGACCTGCTGCTCCT |
| | | GNTGGTNGGNTACAACATGANAAGCTCNNCCTTTCTGCTGCAACTGNCNAATCCNTCNTCANGGAATCAAAGCCCTT |
| Phase-1 RCT-214 | | TATGACATGATTACGAATTTAATACGACTCACTATAGGGAATTTGGCCCTCGAGGCCAAGAATTCGGCACGAGGGTTTC |
| | | TTCTGAGCTGCATGCCCCTTGCTGGTTAGATGACAGCATACTTGACTCCCTCTATGATATTGGAGGTGACAACAACAGG |
| | | AGACATTTCTCCCAGAATAGCATTGGACTTCTCAGGCAGACTCACAGTCTACTGTTTTGGTCTTTGTCTCTCCTCCCCCT |
| | | ACTTCTGTCTCCTGGGTTCATTATCAGAAAGATAATACTAAAGTGAAAGCTTTGTTTAAGGTCTTAAAAATTGAAGAAAAT |
| | | CAGAAATTGTAAAGACAGTAAGACTTCAGACATACATTTTATAAGATCACAGTACAATAGTTAGAAGTACTGATGAGTGT |
| | | ATTCCCAATCCCTGGTCCCTAAGGCTAAATCCACTGCTTGTTCCTTGCTCCCTCGTATACTCTCAAGGTCTCTTTCAAAG |
| | | ATGGTTGCAGTGTTTGTCTCCATTGTTTTTCCATAAAGTATTTCCATTTAAAAAAAAAAAAAAAAAAAAGTGAGCGGCCG |
| | | CAAGCTTATTCCCTTTAGTGAGGGTTAATTTTAGCTTGGCACTGGNCGTCGTTTTACAACGTCGTGACTGGGAAAAACC |
| | | TGGCGGTACCCAACTTAATCGCCTTGCAGCACATNCCCTTTCGCCAGCTGGNGGTAATAACGAANAGGNCCCCACCGA |
| Phase-1 RCT-215 | | ATGACATGATTACGAATTTAATACGACTCACTATAGGGAATTTGGCCCTCGAGGCCAAGAATTCGGCACGAGGGTAATT |
| | | TTGGAGGTTTCCCCACAGCAAGTCACTCTCCTTTTCAGCCCCAAACTACAGGTGGAAGTGCTGGATCAGTAAATGCTAA |
| | | TTTTGCTCATTTTGATAACTTCCCCAAATCCTCCAGTGCTGATTTTGGATCCTTCAGTACATCCCAGAGTCATCAGACAG |
| | | CATCAACTGTTAGTAAAGTTTCAACAAACAAAGCTGGTTTACAGACAGCAGACAAATATGCGGCACTTGCTAATTTAGAC |
| | | AATATCTTCAGTGCTGGGCAAGGAGGTGATCAAGGGAGTGGTTTTGGGACCACCGGTAAAGCTCCTGTTGGTTCTGTG |
| | | GTTTCAGTTCCCAGTCATTCAAGTGCATCTTCTGACAAGTATGCAGCCCTGGCAGAGTTAGACAGCGTGTTCAGTTCTG |
| | | CAGCCACCTCCAATAATGCGTACACATCCACCAGTAATGCTAGCAGCAGTGTCTTTGGAACAGTGCCTGTGGGTGCCT |
| | | CTCCTCAGACACAGCCTGCTTCAAGTGGGCCTGCTCCATTTGGAGCTACGCCTTCTACGAATCCATTTGTTGCTGCTAC |
| | | TGGTCCCGTCTGCANCGTCATCTACAAATTCATTTCAGACCAATGCCAAAAGGANCAACAGCGGCAACCTT |
| Phase-1 RCT-219 | | CTATGACATGATTACGAATTTAATACGACTCACTATAGGGAATTTGGCCCTCGAGGCCAAGAATTCGGCACGAGGCTCC |
| | | ATCCTCAAGTGGACAAGAACTGTGCATCTGTCGTCCTGTGAGCCCAGCAGGACCTGCCCAGGAGGCTCCAGAGTCAG |
| | | TCATGGCTTTCTGTGCTGCAGGCCCTTGACCCAGACAGGGCAGGAATCTCCCCAAAATCCCAGTGAGGGAGGATCCA |
| | | ACCTGTGATCAGACTCCGGTCTTCTGACCCCTGCCTTCCTACTCCTGCATCCTGTCCCATCACAGACAGCCCTCCTCAC |
| | | AGCCTGGTTCATCTGCCTTGTCCTCCAACAGTGCTCTCTTGGGAGACAAGAGATTCAGAAGGGGAGGCAGGAACTCGA |
| | | GCTTGACTTCCACCTGTCCACCTGTTGGGAGTTCTGTCCAATGTGTGACCAACGACAATAAACCATAGCAAGCCGTGAA |
| | | AAAAAAAAAAAAAAAAAACACATGCGGCCGCAAGCTTATTCCCTTTAGTGAGGGTTAATTTTAACCTTGGCACTGGCCG |
| | | TCGTTTTTACAACGTCGTGAACTGGGAAAAACCCTGGCNGTTCCCAACTTTAATCGCCTTGCAGCACATTGCCCCTTTC |
| | | GCNAGCTGGNGGTAANAAACGAAAAAGGCCCCCCACCGAATCNCCCTTCCAAACATTTCCCACCNTNAATGGGCAAAT |
| | | GGGACCCCCCCTTGTAAC |
| Phase-1 RCT-221 | | TTATGACATGATTACGAATTTAATACGACTCACTATAGGGAATTTGGCCCTCGAGGCCAAGAATTCGGCACGAGGCTAA |
| | | CCCTGTCCACGCTCCTGCCTGCAACCCTCTCCCTGCTTGGCACAGTCGAGGAGGAAGATGCTCTTTGCCTATCCCAGC |
| | | TGCACCCTGGCTTCCTGCTCAAGGGAAGTGAGCACCCCACTTCCTGTGCTAGTTAGTGCCTGATTCTCTGGGTGAGTC |
| | | CCCGGGCGGACTCCCTCAGCCCCTTTCTCTGGTACAGTGGTGTCCGCCCGACTGCCTCCTGTAACCCCATCTTCTAAG |
| | | CCATCAATTTTATGTTACTATATTGCCCTTTGTGGGGTGGGAGAGGGATCTCCTGGCTCTGCGACTTGCCCCTTTGCCG |
| | | AATAGTTACTGTTCTTGACTTGAAGAGAAGCAACGTGTGGGGACCTCCCCACTGCCCCAGCCCAGACTTCTTCGGAAG |
| | | GGTTGGAAGTTGCTAGACAAATCAGAATGTAGAAGGTGGAGGATTCTGAGGAGGAGGCAGAGAATTCTGACTGGGGA |
| | | GGTATANGTTGGGTCCTCTGCCTCCCACGGCTGCAANGTGTGTCTGACCTCTGGAGCTCAGCCCCTCCCCCCTTTCTC |
| | | TTCAGTGCTGACAAGATGTCNATAAACTTATTTTCATACAATTAAAAAAAAAAAA |
| Phase-1 RCT-225 | | TCCTTAGGANATGATTACGAATTTAATACGACTCACTATAGGGAATTTGGCCCTCGAGGCAAGNAATTCGGCACGAGGC |
| | | GAAGAGACTGTGTGCCCGCAGNTACCTGTGTTTTCCAATAAACCCTCTTGCTGATTGCATCCGAGTGGACTCGGCTCG |
| | | GTCATTGGGCGCTTGGGACTCCTCCTGAGGGAAAGGTCCTCTCTGGGGGTCTTTCAGTGTTGTGCACGCCCATGATGC |
| | | TAGCACAGTAAGAGGCCAGTTTGGGCTGTGTATCAAGTTCATGGTCTACACGATCTATTTAGCAAACCTTGTCCCATAA |
| | | CTAAAAGGAACAGAAGACTAGAAAAAACATCACCAGGAAGAATGGTCCTTAAATAACTGTCCAGTTAGTGCCAGGGAAT |
| | | AATTATAAAAAGTACTGATGTTAATTCCTCCAGAAACACCCGAAGTGTAACCGTGATTTAAAAGCCTACATTACAAAAAT |
| | | CATTTTCATAGGACATACCTTGCTTTTTTTTTTGGAGCTTCACACATGTGTGAGGAAAGGAAAACAGAACTTAGATGTTC |
| | | CAGTTAAACTTTAACATATTCACTTCTTCAGAGAACCCTATTAAGCCTTGGTCCTATCTGTCAGGTTCCCTCTTTTCTTTA |
| | | TAATTCCTGTTAGCTGCACCACAAACCAATATATGACAAAGGAACACCTTGTATACATGGGCTTGGTACCAACAGCCC |
| | | CTAGAGA |
| Phase-1 RCT-227 | | TCTACATGATTACNAATTTAATACGACTCACTATAGGGAATTTGGCCCTCGAGGCCAAGAATTCGGCACGAGGCAAGAA |
| | | GTTAAGATCTGGAAGGTAGCCACTCTAACTAGGGGCGTGCTGAGACAAGACCACCTCATTCCTCTGCTGCTTTTCAGA |
| | | CAGGACTGTCCTGCCGACCCACCATGATCCAGGCTGCACTGTTCCTTGGCTGTATCTTAGTGTCCTCGGTGACCGCCT |
| | | TTCCATGGAAGACTCAGGATGGTGGCCTGCCCCATCAGCCAGCTGGCACAGAAACTGAGCCTACACAACTGCTCTACA |
| | | GCAAGAGTCCTCCTCCGACCTCCAGTACCTGTCGGAACCTCCTAAGCATGGCGCCCCTGCCCCCTGTAGCTCCTCTGT |
| | | CTGAGTCCTTGTCCCTCTTAGCACTGAGAGTTGTCCTGGAAAACATCGGCTGCCCTGCTGAGGCCTACTCTCTACAGCT |
| | | TCAGCTCAGGGAAATGGGAGGAAAAGACAGCACAGAAACTCTGATTCTCGAGAGTCAAAAGCATAGCCAGGAGGAAG |
| | | GCATTGGCAATAATGAAGCCATTCTGAGGCATCTGGTAGCATCCCCTGGAGAAATAAAAGANGGTCGAGCGCTCAGTT |
| | | ACTGTGCCTGAGGCATGCACCTCTGAACANGGGTTGTGTGTTGCGTGANTTGGCACAACTGGGGGGGGGGGGGGGGT |
| Phase-1 RCT-230 | | TATGACATGATTACGAATTTAATACGACTCACTATAGGGAATTTGGCCCTCGAGGCCAAGAATTCGGCACGAGGCACAC |
| | | ACACACCCCTCATCACTGATATGTTTTATCTTACCCCATCTCTCAGAGTGTTCCCTTAAAACCCAGGCTTTAACCCGGCA |
| | | TGTTCCCTCAGACTGTCTCCCCAGAGCAGAGCAAGGCCAGAGTTCTGAAGTTCTCCATCTCATTTAGTGGCCTCCTCTC |
| | | TTCTCCCAGTTTGAGTCCTGGAGGCCAGGAAACGGGCAAGCTGTTGCTCATGGCTCCGTGGCCTTCTGTTCAGAAACT |
| | | TATTCTTGCCACTGGTTCAATGAATCTCATCACCCTCTCTGCCAATGGGAGCTCTTCTCTTGTGGGAGACACTGTAAACA |
| | | ACACCAAAGGAAAAAATAAAACCACTGTGTGTGTGACTGAAAAAAAAAAAAAAAAAAAAAAAAATGGAAGCGGCCGCAA |
| | | GCTTATTCCCTTTAGTGAGGGTTAATTTTAGCTTGGCACTGGCCGTCGTTTTACAACGTCGTGACTGGAAAAACCCTGG |
| | | CGTTACCCAACTTAATCGCCTTGCAGCACATCCCCCTTTCGCCAGCTGGCGTAATAACGAAAAGGCCCGCACCGATCG |
| | | CCCTTCCAACAGTTGCGCAGCCTGAATGGCGAATGGGACNCNCCCTGTAACGGCGCATTAAGCGCGGCGGG |
| Phase-1 RCT-239 | | TATGACATGATTACGAATTTAATACGACTCACTATAGGGAATTTGGCCCTCGAGGCCAAGAATTCGGCACGAGGGGTCA |
| | | AGTTCCTGCCAAGATGTCCTGCCAGCAGAGCCAGAAGCAGTGCCAGCCTCCTCCCAAGTGCCCCTCCCCGAAGTGCT |
| | | CCCCAAAGTGCCCCCCAAAGAGCACAGCACAGTGTCTGCCTGCAGCCTGTTCCTGCTGTGCTACAAGCTCTGGGGGC |
| | | TGCAGTGTCCCCAGCTCTGAGGGAGGCTGCTGCCTGAGCCACCACAGGCGCAGGTCCCACAGATGCAGGCGCAGGA |
| | | GCTCCAGTTCCTGTGACCGTGGCAGTGGTCAGCAGTCTGGGGGCTGCTGCTGACTTGTATCACGAGGTTGAGACAAG |
| | | AGAGTTTTGAATACTGAGCCACCTTGGCTTGTGCTCTCTCAGACATCCTTATCTCTCTTGAGCTATGTGTTCTTAGACTC |
| | | TTCTAATGGATTTCCCGAAAGCCTGGTTGCCTCTCCTTCTCATGGTTCCATCACCTGTGTCTGGGCATGTCAGTGAATA |
| | | AAGTTCTTTCTCAGCATTACCAGTAAAAAAAAAAAAAAAAAAAAAAAAAATGGAAGCGGCCGCAAGCTTATTCCCTTTAG |
| | | TGAGGGTTAATTTTAGCTTGGCATGGCCGTCGTTTTACAACGTCGTGAATGGGAAAANCCTGGC |
| Phase-1 RCT-24 | | GTTAGNTTTNANATGATTACGAATTTAATACGACTCACTATATGGGAATTTGGCCCTCGAGGCCAAGAATTCGGCACGA |
| | | GGCTGATATTGAGCGCCCCACCTATACCAACCTCAATCGCCTCATCAGCCAGATTGTCTCCTCCATCACGGCCTCTCTC |
| | | CGCTTTGATGGAGCCCTCAACGTGGACCTCACAGAGTTCCAGACCAACCTGGTACCCTACCCCCGAATCCACTTCCCG |
| | | CTGGTCACTTACGCACCCATCGTCTCTGCCGAGAAAGCCTACCACGAGCAGCTGTCTGTGGCAGAGATAACCAGCTCT |
| | | TGCTTCGAGCCCAACAGCCAGATGGTGAAATGTGACCCACGTCACGGCAAATACATGGCCTGCTGCATGCTCTACCGT |
| | | GGTGATGTGGTACCCAAGGATGTGAATGTCGCCATTGCTGCCATCAAGACCAAGAGAACTATTCAGTTTGTTGACTGGT |
| | | GTCCCACAGGCTTCAAGGTGGGCATTAACTACCAGCCACCTACTGTCGTGCCAGGAGGAGACCTGGCCAAAGTCCAG |
| | | CGAGCAGTATGCATGCTGAGCAACACCACAGCAATCGCAGAGGCCTGGGCCCGCCTTTGACCATAAGTTCGACCTCAT |
| | | GTATGCCAAACGGGCCTTTGTACATTGGTATGTTTGGAGANGGAATGGAAAAAAGGAGAATTTTCTGANGCCAGGGAG |
| | | GACTGGCTGCTTCTGGANAAAG |
| Phase-1 RCT-241 | | TCNTNACATGATTACGAATTTAATACGACTCACTATAGGGAATTTGGCCCTCGAGGCCAAGAATTCGGCACGAGGGGA |
| | | GCATGCTGACCTTATTAGTCAGGCTCGCCCTGATTGTAGTACCCCTGAGCTTGCCCTAGGGTAGACGCCAGTCATTTC |
| | | ACAGAGCCCACCTACTCAGAACTGCACAACACGGCTCTGGTCCTGGGATTTTGGTTTGCAGTGAGAAGAAAACATCTT |
| | | CGTGTAAACTTGACCTCACGCTTAATAGTTTAAGGGTGAAAACAAGTGGATCAGATGTGTGATGAATGCCACAAGAGAC |
| | | TGATTGCTTTTATTGTATAGATTTATCACATTTAATGGAGAGGTTACAAACTAGACTTTGGCCATACCTTGAAATGAGAAC |
| | | TTGTAGTCACTGATAAGTACAAAGAACTCTCTCGTGTCTACACTAAACCTAACCAGAGATGTAGAAATGGAGAGGAG |
| | | GCTCTTCACAGGTCTCCCACTGTCTAGCTTATGGCAGTAAACCAGGAGAGCAAAGAGCTCCACGCTTACTGTCCAGGT |
| | | GACTCTACCCTGTTCAACAGACTTCATTCTTCTTTAGTTGTTGACTACCTCAGAAGAGAAGAAAAGGACATTTCACAGAG |
| | | TGATGCCTTTTACACATAATATACCCCCCACACACACACACACGCTGTAACTAGAAGG |
| Phase-1 RCT-242 | | CATTATGACATGATTACGAATTTAATACGACTCACTATAGGGAATTTGGCCCTCGAGGCCAAGAATTCGGCACGAGGGA |
| | | CCGAGAGCTGTGGGTTGAAGCAAAGCTGTGAATCGCTCCAGATGGTCCCTGTGTTCTGTCCACACACAGGTCCCCGC |
| | | CTTTTAGAAGCAGCCTCCTGGTCTCATGCTTAAATCTGTTCCTCACTGCCCGTGTTCACTTTANAAATGGCAGAACCAC |
| | | AGAGCTGGACTGTTGAGCAGGCCTGTCTCTCTCATTAAATAAAAATAAGTAAGTTTGTAAGCTATTCCGACAGAANAGA |
| | | CAAAGGTTACTGATTGTACAATAGCGCTTTTATATGGAAGACTGTACAGCTTTATGGACAAATGTAAAACTTTTTTGTTTT |
| | | TAATAAAAATGTAGCAGACCCCAANCNTAAAAANAAAATAAAAATGGAAGCGGCCGCAAGCTTATTCCCTTTAGTGAGG |
| | | GTTAATTTTAGCTTGGCACTGGCCGTCGTTTTACAACGTCGTGACTGGGAAAACCCTGGCGTTACCCAACTTAATCGCC |
| | | TTGCACACATCCCCCCTTTCNCCAGCTGGCGTAATAACNAAGAGGCCCGCACCCGATCNCCCTTCCAACAAGTTGCNC |
| | | ACCTGAATGNCGAATGGGACCCCCCCCTGTACCGGCCCCNTTAAACCCGGCGGGGTGG |
| Phase-1 RCT-248 | | TTATGACATGATTACGAATTNAATACGACTCACTATAGGGAATTTGGCCCTCGAGGCCAAGAATTCGGCACGAGGGGC |
| | | ATTATTTACTACTCAATTTTGCCTTACCAACCTCACTGATAATAATGAACACCGATTAGAGAAAGAATGCTAGAGGAAAG |
| | | ATGGTTGTGGCCTCCCCAGTCTGTCATCTGAACCCTTCATACATGGTGATCTAGAACATTATTAGCCACGGTGATCTTC |
| | | AAGGAGGCAAACAAGTCCTGTCTGCTTCTCTTGGCATCTGCTGACTCTAACAAGACCGTTTCAGAACTTTCCACTGGAA |
| | | GTCCTCTTCCTCTCAGAAGTCAGAAATTCTCCCTAGAAGAATAAGAAATAAATTCTTTTGGTAACCTGAAAAAAAAAAAA |
| | | AAAAAAAAAAAAAAAATTGGAAGCGGCCGCAAGCTTATTCCCTTTAGTGAGGGTTAATTTTAGCTTGGCACTGGCCGTC |
| | | GTTTTACAACGTCGTGACTGGGAAAACCCTGGCGTTACCCAACTTAATCGCCTTGCAGCACATCCCCCTTTCGCCAGCT |
| | | GGCGTAATAGCGAAGAGGCCCGCACCGATCGCCCTTCCAACAGTTGCGCAGCCTGAATGGCGAATGGGACCCCCCTG |
| | | TAGCGGCGCATTAAGCGCGGCGGGGNGTGGNGGNTACCCGCAGCGGGGACCGCTACCACTTGCCAGCGCCC |
| Phase-1 RCT-252 | | TATGACATGATTACGAATTTAATACGACTCACTATAGGGAATTTGGCCCTCGAGGCCAAGAATTCGGCACGAGGGGCC |
| | | AATACTCTACTGGCAAGACCACCTTCATCAGGTACCTGCTGGAACAGGATTTTCCAGGCATGAGGATTGGGCCTGAGC |
| | | CAACCACTGATTCCTTCATAGCAGTGATGCAGGGAGATGTGGAGGGGATCATCCCTGGGAACGCCCTGGTGGTGGAT |
| | | CCGAAGAAACCCTTCAGAAAGCTCAACGCCTTCGGCAATGCCTTCTTGAACAGGTTTGTGTGTGCGCAGCTGCCCAAC |
| | | GCTGTTCTAGAAAGTATCAGTGTCATCGACACAGCGGGGATCCTCTCTGGTGAGAAACAGAGGATCAGCCGAGGGTAT |
| | | GATTTTGCTGCTGTCCTCGAATGGTTTGCTGAGCGGGTGGACCGAATCATCCTGCTCTTTGACGCCCACAAGCTTGAC |
| | | ATCTCTGATGAGTTCTCAGAAGTCATCAAGGCCCTCAAGAACCACGAGGATGCAGGATCAGCTGCAGGCCCAGGACTT |
| | | CAGCAAATTCCAACCACTGAAGAGCAAGCTGCTAGAAGTGGTTGATGACATGCTGGCCCATGACATTGCCCAGCTCAT |
| | | GGTGCTGGTACGCCAGGAAAAGACCCACGGCCTGTTCAGATGGTGAANGGCGGAGCATTTTGANGGAACCCTCCAAG |
| | | GGCCCTTTGGGCATGGGCTTT |
| Phase-1 RCT-256 | | TCTCNTAANATGATTACGAATTTAATACGACTCACTATAGGGAATTTGGCCCTCGAGGCCAAGAATTCGGCACGAGGAA |
| | | TTGAGTGACATATCACTCCTGAGTATGCCCACTAGATGCGGTGGAGATGCAGAGGCATCCGGACCCCACGCCCCACC |
| | | CCCTCCCCTCACACACTTACTCTCTGCCTAGTAATGCCACAGAGCTTCCATCCCCATCCAAAGGTCATCAGGCATGGCT |
| | | ATCAGTTGGCTCTCAGGGTGGATTTGACATTCTCAGATGATTAGAAGTTGGCAAGAAGCAACCTTGGTGAATAACTCTG |
| | | GTGTCTAAACTCTGTACTTGAGTTACAGTGTCAGTAGAGGAGACGCCAAAGCTGTTGCGAGTGACGGCAGGATTATTG |
| | | AACAGTCATGATGCTTGGCTTTCAAAGGCGATTATCGCTTTAAGGTCTTAGAATTAGTAAGTGCATCTTTATAACCAGGC |
| | | ATAGCTAGATCATAACTACTGATGGCCAAGGACCATAGAACGTGCTTCTTACCTTCCTCTCTAGTTAGGATTACGACAA |
| | | ACATAATCACCAACGCTCAGGGAAACACTTGCTGATTCAAGTAAAATGCATGAACCTTGGAAGACCTTTCTAGAAGTCA |
| | | GAGATCAAGTTCATCTTGNTCTAGCACTTTCCACATTCATGTTTGGGTTGTATGCTGCGCCCTAN |
| Phase-1 RCT-258 | | TNATGACATGATTACGAATTTAATACGACTCACTATAGGGAATTTGGCCCTCGAGGCCAAGAATTCGGCACGAGGACCA |
| | | GTCAGGGAAGAATGTGATGGTGGAGCCCCATCGGCATGAAGGAGTCTTTATCTGTCGGGGAAAGGAGGATGCCCTTG |
| | | TCACAAAGAATCTGGTTCCTGGAGAATGTGTGTATGGAGAGAAGAGAGTCTCTATCTCCGAAGGAGATGACAAAATTGA |
| | | GTACCGAGCCTGGAACCCCTTCCGCTCCAAGCTGGCGGCAGCAATCCTGGGTGGCGTAGACCAGATCCACATCAAGC |
| | | CGGGGGCCAAGGTGCTCTACCTTGGGGCAGCCTCAGGCACCACCGTCTCCCACGTGTCTGACATTGTTGGCCCGGAT |
| | | GGTCTGGTCTACGCAGTTGAGTTCTCCCACCGCTCTGGCCGTGACCTCATCAACTTGGCCAAGAAGAGGACCAACATT |
| | | ATTCCTGTAATTGAAGATGCTCGGCACCCACACAAATACCGCATGCTTATTGCAATGGTGGATGTCATCTTTGCCGATG |
| | | TGGCCCAGCCAGAGCAAACCCGAATTGTGGCCCTGAATGCCCACACCTTCCTGCGGAATGGANGACACTTTGTGATTT |
| | | CCATTAANGGCCAACTGCATTGATTCCACAGCCTCAACANAANCTGTGTTTGCATCTGAAGTGAAAA |
| Phase-1 RCT-264 | | CTANNCCNTATGACATGATTACGAATTTAATACGACTCACTATAGGGAATTTGGCCCTCGAGGCCAAGAATTCGGCACG |
| | | AGGCATCTCGAGCAGAGAGCCTGTTTCCAGCACCCGTTCTCCTACCACCAAGTCACACTCGTGGCTGGCACAGAGAAG |
| | | GTGCTCACTGTTTGCTTGTTCAGACCGCGTCAGCAAAACATGGAAAACTCATTGAGTTTAATATTCTGGGCTTAACAGAC |
| | | TGATTATTTTCAGGAGCAAACTGAAGGATATTCGTAAGTATGCTGAAGTGACAGGTGGAGAACAATTCCCATTAATTATT |
| | | ATGTCTAATTATTCCACTTAATAATGATGAGATGCAAATAAGACCAACCAATAAAAAATGAGGAAAATACACAAGTATAAT |
| | | GTATAGAAAAAGCACAAAGTATTACCATTCCTTCAGCTTCGAACAAGACCATGATCAACATCAAAGGACAACCTATAGC |
| | | CCAAGACATGTGCCTGTCTGCACTCCAGGCTTGCTTACCTTGCTACGGATGATGAGAGGGAGTGGCAATAAAACCAAA |
| | | ACAGTGGAAAACCACAAGGAGAAAGCGACGATACACCAAAAGCGTAATTGAGGAGCTTCATGCCTGAGCAGGTGCTTC |
| | | AACANTTCCCCCTCACTCTCAGGCAGAAGTTAATAACCAGCTGGGATTAATATTTCTCTACGTCATCATCTTTACCTACT |
| | | GGCTCAGANAGAACCAACGCTGGTTAAAAATAAATCTCATTTTTATTGGTTTN |
| Phase-1 RCT-270 | | CTATGACATGATTACGAATTTAATACGACTCACTATAGGGAATTTGGCCCTCGAGGCCAAGAATTCGGCACGAGGCCCA |
| | | GCTGGCCTCTCGGCTCTTCTCCTCTGTCCTGTTCTCTACCCTGTGCTAGTCTCCCCTCTCACAGATACCCCTGGCCACA |
| | | TTCAGTCTGCTCTCTGAACTCTTCAGATGCCTCTGCCTGTCCTCTGGTATCTAAAATAAGAACTTCCCCTCAACCATACC |
| | | CTGTCTGTCTATAGCTCCCCCAGGGAGCTTGTGTTTGAAGTCTGCTCAGCTGCATGGTGGAGAGGACTGCATTCGTTT |
| | | CTCTTGCTCACAATTCCTTTCAAATTGCATTGAGAGCCAGAAGACAAAGGAAAGAACTGTTAACTAGCAGAATGCATTTT |
| | | ACAGAGCTCTGGCTGACAGTCTCCTTGCCCACGGAATCATTTGTCTCAGTTAGCGTTTAAAGGAGTTTTTGCGAGGCAA |
| | | ATTTCCGTTTGTGAGATCTCTGAAGTGGGATTGTTATTTTCCCACTGCCAAAACACTTTCACTGATTTCAAATCAGGAT |
| | | GCATTACAGAAACTATTTCCTAGGACTTCCTCAAAGCTAAGCAAGATGCCCCAGGCCAGTGGCAGGCAAACAGGATGG |
| Phase-1 RCT-271 | | CTATGACATGATTACGAATTTAATACGACTCACTATAGGGAATTTGGCCCTCGAGGCCAAGAATTCGGCACGAGGCACA |
| | | TTTCTTCAGTCATCATGGAGAAGGTGGGCACTAGCACTACGATACCACCCCAGACACAGTGGAGTATCTCGGCTACTTC |
| | | TCGCCTGCACAGTTCCTCTACCGAATTGACCAGCCCAAGGAGACCCATGCCTTTGGGAGGGATTAAAGCAGCTGCTGC |
| | | TTGATTAGGGGAAAAATGGTTAGCTAAGGTTATATTGCTAACCCAGCAATTGACAGTAATTAAAAAATATAGTAGGACCA |
| | | AAGAGAGAGAGAGAGAGACCGCAGCCAACAACAGTCATTTCACTACATTAGCACTGTTTTTATGGCATCCATGTGGATA |
| | | AGGGACACTCGTGTTATTTAGCATCTTTATGTGAAGGAGTGTTGGGAACAAAGATAACTAATTGAGATGCTCACTTTACT |
| | | CAACCAATTTTCTGTCTTTATAGCCCAGAAGTCCACCTTCCTTCTCTTCACACCTCAGGTATGGGCATGGACAGGGACC |
| | | TTTCATTATCCCTGCTATCGAGTAACATTTGGGTCACATTGCTGAATTTTTCAAGTTCATCTCACAAGGCTAAGGGGTC |
| Phase-1 RCT-277 | | TTCTATGACATGATTACGAATTTAATACGACTCACTATAGGGAATTTGGCCCTCGAGGCCAAGAATTCGGCACGAGGCT |
| | | GAGAATCTGTATTGTGGTGTATAAAGTGTGTCCTAAGAGCAGTAAAAGGACAAATTAAGCAGACTTTCTTTTCAAGCTT |
| | | ATGACTTAGATTCTTTTAGAAATATAGTTCTTAATCTTTAAAGATGAGATTCTAAGCCTAGAATTTTAAACCACATTTCATT |
| | | ATGGTGGCTTACTGATCTCAAATTTTCTACAATTTGGTTTTTTTAGACTTAAAAACAGCTAAACAAAACCTTCCTTTATCTT |
| | | TCTCATCACATCCAGTGGCAGAGGGGACTGTCTGAGAGTAAAGTCCCATGATTCAGAAGGACATAGCAGGTCAGGATG |
| | | TACCAATAAAAGTTATGAATGTGATGTTCATTAGAAAGAAACTGAACAACTGAGTACAGAAATGAATATCAGCAAGAATT |
| | | TGTTTTAAGGATTTTCCAGATTTTACTTTGTATCTTGAAGTGTGTGCCATATGCCAAGAACTAACAGAACTTCCTGTTCCA |
| | | TCTCTGTACCTTCTTCTTTTTTAAAAGTGAACTTATGGGTCGGTTCATTNCTGGGGGCCTTATGGGATGGGTTGGCTTG |
| | | GACTGGTTGCCACTGGATGCTGCAGTTNCTTTTAAT |
| Phase-1 RCT-278 | | TNTATGACATGATTACGAATTTAATACGACTCACTATAGGGAATTTGGCCCTCGAGGCCAAGAATTGGGGACGAGGGCC |
| | | CCTTGCTGGCAGTGTGCAGAAAGCAGAGGTCTAGGAGCAGGAAGCTCCTGTGAGCGGCAGCTTCCAGGACTTCAGGC |
| | | CTGGGTATACCCCACTGCTCACCCCCCTCCAGATCTGCTGATCCTCTGGTCTCAGGTCAAGGACCCTTTGTGGCCGCG |
| | | GGAGCACTATGGTCGTTCAGCAGGCACACCTTGGGTTAGCCAGGAGCCACATCTTTCTGTGTCAGAGCCTTCCACTGG |
| | | GCTGCGCGATGCCCTCTGTAGTTCCTCTGAGACGCCCCTGCCCTCAGTTTCCCCTTTCATCTGACTTCGGCTGCCCTCT |
| | | CCAGCCCCAGCCTTTGGAACTCACTCTAGCAATACCACCAGACTAGTTAGCCTTCCCGGCCCCCAAGACACTTGCGGT |
| | | TTCATGCCTTTGTGCCTTTCGCTCACGCGGTTTCTTCAGCTGGAATGCCTTCCCGCTCCTCCCGCCTCCTCTGCCTGGC |
| | | AAGCCCCTATTAGCCTCTCCCAGGCCCCTCCCTCCCCCAGGAAGGCACCTCCACCCCTCTCTTCCGGGCTACTCTGCA |
| | | C |
| Phase-1 RCT-285 | | GNNNNNTNCTATGACATGATTACGAATTTAATACGACTCACTATAGGGAATTTGGCCCTCGAGGCCAAGAATTCGGCAC |
| | | GAGGATCGTGGTTCTTTCAGATGCCTTCCTCAGACGTCCCTGGTGTAGCCAGAGCTTCCGGGAGGGACTGTGCCGCC |
| | | TACTGGAGCTCACCCGCAGACCTATCTTTATCACCTTCGAGGGCCAGAGGCGTGAGCCCATTCACCCTGCTCTCCGTC |
| | | TCCTGCGCCAGCACCGCCACCTCGTGACCCTGCTGCTTTGGAAGCCTGGCTCCGTGACACCTTCCTCTGATTTTTGGA |
| | | AAGAGCTACAGTTGGCACTACCACGGAAGGTGCAATACAGACCCGTGGAGGGAGACCCCCAAACCCGACTTCAGGAT |
| | | GACAAAGATCCCATGCTAATCGTGAGAGGACGTGCTGCCCAGGGCCGGGCCATGGAGTCAGAGCTGGATCCAGACCC |
| | | TGAGGGAGACCTGGGTGTCCGTGGACCTGTCTTTGGGGAGCCACCAACTCCACTACACGAAAGCAAGGTCTCCATAG |
| | | GAGAGGGCCATGCCAGTGAAATGGATGTCTCTGACCTTGGCTCTCGAAACTACAGTGCGCGAACAGACTTCTACTGCC |
| | | TTGTGTCTGAGGATGACGTG |
| Phase-1 RCT-287 | | TATGACATGATTACGAATTTAATACGACTCACTATAGGGAATTTGGCCCTCGAGGCCAAGAATTCGGCACGAGGCCAAA |
| | | ACCTGACGGACAGATCAGTTTTGACCTCTTATCCTCTGTGGCTCTGAGTGGTACTAATCATGAACATGACCAGCCAGCA |
| | | CATTTAACCTTGAAGGATGACAGCATACCTGTTAATAGAAATCTGTCAATATATGATGGGCCTGAGCAGCGATTCTGCC |
| | | CTGCAGGAGTTTATGAATTTGTTCCTCTGGAACAAGGTGATGGATTTCGGTTACAGATAAATGCTCAGAACTGTGTGCA |
| | | TTGTAAAACATGTGATATCAAAGACCCAAGTCAAAATATTAACTGGGTGGTCCCAGAAGGTGGAGGAGGACCTGCTTAC |
| | | AATGGCATGTAAAGCCCAAGTGCCTCCACTTACTGGCACACTTGACAGCCAGTTTCTAGAATACTGTAAATGTATGCCA |
| | | AACTAACCTCCCATATGTTTGGATAACTTCTGAACAAGTGTCCTTCAAACACTGAAGTAAAAAACTTTGTATCTAACGTC |
| | | CCATAAAATCATGAAATATTTGTCATTAATAAAACTTTATAAATAAATAAAAAAAAAAAAAAAAAATCTCGCGGCCGCAAG |
| | | CTTATTCCTTTAGTGAGGGTTAATTTTAGCTTGGCCTGGCCGTCGTTTTTCAACGTCC |
| Phase-1 RCT-288 | | TCCTNATNAGATGATNACGAATTTAATACGACTCACTATAGGGAATTTGGCCCTCGAGGCAAGAATTCGGCACGAGGAA |
| | | TTAAAACTATATAAATAAGAATAATAGAAAATAAGTTAAGGAAATAAACAGTAGTAAAACACCTCTGGTTTATCAAACGTT |
| | | TAATCATAGAAGGAAACCCTGATGTCACTTTCTTTACGTATGACTCTGTGAAGGAATGTAATGGTACATTACAATAGGAG |
| | | CTAATGTTTTAAATGTGTACAGTAGTGAAATAATTAACAATAAACTGGAGTTCAAAATGCCAGTCAATGTAAGTACATTCT |
| | | ATGATGGGGCTTTGAAAGTGTTTATTCCATGAAGCAATTCTACAAAGAACATTGATGAGCAATATGGGTAAACTGTTTGG |
| | | AAGGTGCTGGGCAAATAACTGGAATTGTCTAAGTGGCTTCACCGCACTGTACCAGAAACATATTCTGAAAGTCAGATCC |
| | | ATCAGTGCTCACTGTGCTGCCGAACTTCACAGTAATTTACTTTACTGTTGTGAAAAATAAACATCGCTCTTGTAAACTGT |
| | | GGTGTTAACATTTTTTCTAAAATGTAAAGGAGGCATTCTTTTTACAAAAGAGAAATGCTTTATCTTTCAGAAAAAAAATGA |
| | | AGCAAAAACATCTTATCCATTATCTGAATGTTGATTCCTTTGCTTATAAGTTTTAGGTGCAGGTGGCTTCTCCTATTGCTT |
| | | CTCTT |
| Phase-1 RCT-289 | | GTTACTTGTGGTGGCACAAACNTCAAACAACCAATATACAAGTGTTCTGGGGTTGGATCAGGGGTCGGGGAAGTCCCA |
| | | AGTTTTAACTCTGTGGGGTTTGGGGAGACAAGGTGGGGNAATTGAACGAATGGGGAAATCAATTTATTTTTNTTAATTC |
| | | TGTCCATATAAATATATTCATGAAGACCAAAAGAGGGAAGGGCAGTTGGGNTGGTGATGAAGTGGGAGAAGGGNAGG |
| | | CAGAGCCCTCTCAACTCTACTCAGCCAAAAATATNAAACANATTAATTTCATGGTGGGAGAAGAGATTTAAANAATGATA |
| | | GAAGATGGGAAGGAGGGGGAGACAGAAGGGGACCAACCAGGGAAAAGGGGGACCCATGGCAAGGGAGTCCCATGT |
| | | CAAGGAGTCCTGTGCCGGTGTGAGAATCTGTCTGCTTCTCTCNTCAGCCATAATGTGGTAAGCTCTGGCCCAATCCGC |
| | | CTTCGGCTCCCGGCTTGGCCCTTGCTCCTATTGTGCCAGCCCCTCCGGCCTCCAGCTATTGAGAGCTAGCTCGCTCCA |
| | | GGCCTCGTGCCGAATTCTTGGCCTCGAGGGCCAAATTCCCTATAGTGAGTCGTATTAAATTCGTAATCATGTCATACCN |
| | | NNC |
| Phase-1 RCT-290 | | CTATGACATGATTACGAATTTAATACGACTCACTATAGGGAATTTGGCCCTCGAGGCCAAGAATTCGGCACGAGGTCTG |
| | | AAGTGGGAGTCTGTGAGTTCTAAGCCAGCCAGGGATACAAACTGAGAGCTAGTTTTAAAATAAAATATAGACATCTTCTT |
| | | CGCAGGTGGGACGGTGAAGCCTTGACTCCTGTCTAAATTAGTAGGGGACAAGAAAGTGAGACCTTGGCAGTCCTTACT |
| | | GGCTCCTGCTACAGGCTTAGCTGAAGTATCTGTTCCAAGGGAATTACCTTCCGAAGCCAGCCAGGACAGGAGCTGCTA |
| | | GCCCCACAACTGTTTCCCACAGCCTCTGTGGGGCTGATTAAACCTTCCATTCCACGGTCTGCTTCCATTGTTCCTCGGA |
| | | AACCTCCACTTGGATCTGGAGTTTCAGATTCCTGCTGACTATCTTCCTCTTCACCCCCGCCACCACCAATGCGGCCGCA |
| | | AGCTTATTCCCTTTAGTGAGGGTTAATTTTAGCTTGGCACTGGCCGTCGTTTTACAACGTCGTGACTGGGAAAACCCTG |
| | | GCGTTACCCAACTTAATCGCCTTGCAGCACATCCCCCTTTCGCCAGCTGGCGTAATAGGGAAGAGGCCCGCACCGATC |
| | | GCCCTTCCGAACAGTTGCGCAGCCTGAATGGCGAA |
| Phase-1 RCT-291 | | GTCATCTTCAGCTATGCAGTGAATATGAGGCCAGTCTGGACTACAGGAAACCNTGTATTGGACAGAGCTAGAAGATCAT |
| | | ACAATCAGGAATGTGGGTGTAACAGCACTTACTTTTAAGGATAATGGATAAAACTCGAAAGAACATGAATTCGGAATGG |
| | | TCTAGTTTTAGTGGTGTCTGTTGGAACATTTTGTAGAGCGGCTCTAAGATGACATTTAAAACGAAAATACTGCTGACTTT |
| | | AAAAGGGAGGGAAAATATGGAAAGTTACATGTAATAAACCAATTAAGAGGTAGTGTTGGGGCTGCCTCTACAGAGTGC |
| | | CACGTTCTGGCCAAGAATGTTCTCTACTCATTTAAGGTCAGTTCCAGTACAGTCAGAATCCAACTGCCTCATGACCTCCT |
| | | CTGCCACTTCACTCACATATAACTAAAGCATGACAAACACTATGGTCCTGAAAAGTGTGAAATCTACTGTCTGTTTCATG |
| | | TGCTTATAAAAAATCAACTCCCCTGTGTATCCCACACGCTCCAGATTCAGTTGTGCAAATCAGTCCAGAATTTCAGAGGA |
| | | ACACACCTCGTGCCGAATTCTTGGCCTCGAGGGGCAAATTCCCTATAGGAGTCGTATTAAATTCGNATCANGTNAATCN |
| | | NNNG |
| Phase-1 RCT-292 | | TCTATGACATGATTACGAATTTAATACGACTCAGTATAGGGAATTTGGCCCTCGAGGCCAAGAATTCGGCACGAGGCAC |
| | | GTACTAAGCAGACCGCTCGAAAGTCCACGGGCGGCAAAGCCCCGCGCAAGCAGCTCGCCACCAAGGCCGCCCGCAA |
| | | GAGCGCTCCGGCCACCGGCGGCGTGAAGAAGCCCCACCGCTACCGTCCCGGCACCGTGGCTCTGCGCGAGATCCG |
| | | GCGCTACCAGAAGTCCACCGAGCTGCTGATCCGCAAGCTGCCGTTCCAGCGCCTGGTGCGCGAAATCGCGCAGGACT |
| | | TCAAGACCGACCTTCGCTTCCAGAGCTCGGCGGTCATGGCCCTTCAGGAGGCCAGCGAGGCCTACCTTGTGGGTCTG |
| | | TTTGAGGACACCAACCTGTGCGCCATCCACGCCAAGCGTGTGACCATCATGCCCAAGGACATCCAGCTGGCCCGCCG |
| | | CATTCGTGGAGAGAGAGCTTAAACGGTCCTACGAGCAGTTAACCCAAAGGCTCTTTTCAGAGCCACACNANTNNATNA |
| | | NTAGNAANNNNAANAAAACAATTGCGGCCGCAAGCTTATTCCCTTTAGTGAGGGTTAATTTTACTTGGCACTGGCCGTC |
| | | GTTTTACAACGTCGTGACTGGGAAAACCTGGCGTTACCCAATTTATCCCTTGCAGCACATCCCCCTTTCGCCAGCTGGN |
| | | GTAATAAC |
| Phase-1 RCT-293 | | GNNNTGTCTATGACATGATTACGAATTTAATACGACTCACTATAGGGAATTTGGCCCTCGAGGCCAAGAATTCGGCACG |
| | | AGGCCTCGCTCCTCAACTTGGCAAAAATGCCTACAGAGACTGAGAGATGCATCGAGTCCCTGATTGCTGTTTTCCAGAA |
| | | GTACAGTGGGAAGGATGGAAATAGCTGTCATCTCTCCAAAACTGAGTTCCTTTCCTTCATGAACACGGAGCTGGCCGC |
| | | CTTCACGAAGAACCAGAAGGACCCCGGTGTCCTCGACCGCATGATGAAGAAGCTGGACCTCAACAGTGATGGGCAGC |
| | | TAGATTTCCAAGAGTTTCTCAACCTTATTGGTGGCTTAGCTATAGCATGCCATGAGTCCTTCCTCCAGACTTCCCAGAAG |
| | | CGTATCTAACCCTCTCCATTCCCTTCCAGCCACCAAGTCATCGCCTCCTCCACTCCTTCCCCCATCCACACCTGCACTG |
| | | AGCCCACCACACCTACCACACATGCAGCCCACGCCTGACAGGGAAAATAAAACAATGTCATTTTTTTAAATGTAAAAAA |
| | | AAAAAAAAAAAAAAAAAAATGGAAGCGGCCGCAAGCTTATTCCCTTTAGTGAGGGTTAATTTTAGCTTGGCACTGGCCG |
| | | NCGTTT |
| Phase-1 RCT-296 | | TATGACATGATTACGAATTTAATACGACTCACTATAGGGAATTTGGCCCTCGAGGCCAAGAATTCGGCACGAGGATAAC |
| | | ATTAATCTTTTTAACAAAAATATGGTCACTTTATCTAAACCCATCCTATCCCCAAGTTTAACCAGGATAAGCTATTTTCATT |
| | | GCCAAACTATCTATTCTCCACACCTTTCTGTTCTGATTTTCTGATATTCCTCCCTGTAAATCTGAATAAATTCAGCAAGTA |
| | | ATAACAATGCCACAATTTAAATAATGTTTTCTTGAAAGGAATCATCCAGGGAATACCTTTCCCTCTAACTTCTTTTACTTC |
| | | ACCCTGAACAGGCAGGTGAACCTATACATCCCGAAATTCTCCATATCTGATACCTATGACCTTAAAGACATGCTGGAAG |
| | | ACCTGAACATTAAGGACTTGCTCACCAACCAATCAGATTTCTCAGGCAACACCAAAGATGTTCCCTTGACATTAACGAT |
| | | GGTCCACAAGGCCATGGTACAACTGGATGAAGGGAATGTGTTGCCTAATTCTACCAACGGGGCTCGCCTACACCTGCG |
| | | CTCTGAACCACTTGACATGAAGTTCAACAAGCCCTTCATCCTCCTGCTCTTTGACAAGTTCACATGGAGCAGCC |
| Phase-1 RCT-33 | | TTCCNAGGCATGATTACCAATTTAATACGACTGACTATAGGGAATTTGGCCCTCGAGGCCAAGAATTCGGCACGAGGG |
| | | GAAAATCCAGGGACAATCAAAGTGGGAGACCCTGTGTACCTTCTGAGCTAGTGATGGGAACTGTGGGTTCCGGAACAA |
| | | CAGATGTCTCTTAAAAAAAAAAAAAAGTTTTAAAAACTGACACCGCTGGAAATCGTTCTTCAGCCTATTCTTTGGATCTGT |
| | | GGGTTCCAAGTTTCTCCTCTTTCAGATTTCCTTCTGTTTCAGTGTTTGCTGGGGGCAGCACGGAAAGCAGGGAAATACG |
| | | GTCTGGCGAATTTAGGCCCCCATTAAGTTTCTTGAAGAATGAAGGGATTATCACATCGCCCTGTTTATAATTATGGAGTA |
| | | ATTTTTCTATTTCGGCATTCACTTGCCAAGACAGCAGATGTCCGTCATCTCCCTAGCCTTCTTTTGGGAAAGAGGAGAAA |
| | | AAAGGAGGACGACTGTGTAAGCCAGAAGAATGTTCCAGAATGTTTTGCTACCCCTGGACATGGTGCGTACAATGGAAA |
| | | TTAAATACTCTCCCAAGTAAAAGCTGGAATGACGGCATTCACTTTTCTCCTCGGGCCCAGGCTTTGGAAAGGGTTTAAC |
| | | GAGCAAATTCCGAAGATACAAATTGTTCCATAGATCN |
| Phase-1 RCT-34 | | ACTAAANNAANNCTCNTATGACNTGNTNACGAATTTAATACGACTCACTATAGGGAATTTGNGCCCTCGAGGCCAAGAA |
| | | TTCGGCACGAGGCAAAAGAAACTACAAATCCTAGATTCGTCTGAATATACAGACTCAGAGAATATTTAGTTTCATCTGAA |
| | | AAGGAAAACCTTTTCCNTCTATGTACACCAAGAGCTCTGTCAAATGTCTGCTCTCCATTACCACCTGTCTGACCTCTGCT |
| | | GAGAACAGTCGTCAGTGCAGCCACCAGGTTTCCGCCCTCTCTCAAGTTTGCTGTAGCAGATTGGCAGACTGTCTTGTA |
| | | ACAGCCATTCATTTCATTCCAGATTTTCCCAGGGGAATAGTCTGCATCCTGCTTGCTTTCTGTATAAAACTTACAAATCAA |
| | | TCATGAAATGCNCTAATTTTTGTGAATCAGGACCCTAAGTGTTTAATTGNAAAATAATTTTGTTTCATAGATTGCTTTAGG |
| | | TTTGTGTTATCTTTTTAATTGTTAAACTGNAATTTAACTACNTTGTAAAGTAACTGGAACTGCAGATTAAAGCCAAGGCTT |
| | | TACAGATAAAAAAATAATTTACTGGNAATGNCTAAATTTTGGAAAGAATGGCAATTAAAGCCCACTTTCAGGCTTAAAGC |
| | | CTTTTAANACCACCNNTANTCATCTACACCTCNCCCNTCAAGNNGTGGGCGGCCCCCCANANCCTTTATTTCCCCTTTN |
| | | ANNGGAGGGGGTTAAATTTT |
| Phase-1 RCT-36 | | TTATGACATGATTACGAATTTAATACGACTCACTATAGGGAATTTGGCCCTCGAGGCCAAGAATTCGGCACGAGGGCG |
| | | CGCAGGGGAGCGTGCCCACGGGACTGTTGGGCCGAAGTCTACCTGGTTTTAGAACGATCCAAAGAAATGGCAGGAAT |
| | | GCATTCACTGTATTCCTCTTTCTAGCCCTGCTCCAGATCTCTCACAATGAACTGCTACCCGCAAACAATTTATAGTACTC |
| | | AATGGCAGACATCTGTCTCCTCCCAAACGAACGGTTGGCTGGAGGACTCTAACATGCCCCAACTGTGCACACCTCCTC |
| | | CTTTAGATTAAACCTCAAGGGCTCCCTTCAGCTGAGTGCAATGTGTCTGATTCACCAAGAGATCTCCAGGGTTTGCAGG |
| | | GAGTTTAAGGTCAATGTGATTTTCAATATGGAGCTAAGGAGAGAAATGACACAAATTTAGTCATGCCTTCTATATACACA |
| | | CACCTATGAGCAATAAAAATGACACTTTTCCCGAAAAAAAAAAAAAAAAAAAAAAAAAAAAAAGGTTGTGCGGCCGCAA |
| | | GCTTATTCCCTTTAGNGAGGGTTAATTTTACTTGGCACTGGGCGTCGTTTTACAACGTCGTGGACTGGGAAAACCCTGG |
| | | CGTTACCCACTTAATCCCTTGCAGCN |
| Phase-1 RCT-37 | | TATGACATGATTACGAATTTAATACGACTCACTATAGGGAATTTGGCCCTCGAGGCCAAGAATTCGGCACGAGGCACCA |
| | | CCCCTCCCCCAATGTATTTATTGCACATCTGCTCTGTCTGGCACAGATGATAGATAGTGCTAGTTCATTTTTTTTTAATG |
| | | GAGAAGGCTGTTTTGAGCCCTGTTTCATTCACCTCTTGGTCCAGACTCCCTGAATTTGTTAGCAACTCCTGAGTGGAAA |
| | | GGAAGTCTGAGTGACCAGTGGGACTTTAGAGGTCCACTGGGTATGACTACGAGGAAATTACTGAAAGAGACTGGGGA |
| | | GTTTTTAACCCTTGGAAAGGAATGGCTCTTTCCATTCAAAAATACCAAAGAGCAGAGATAAACCCAACATTCCAAAAGCT |
| | | GTGATTCTACTAATGGGAAGAGGGATGGCTTTGCCTATCTCTCAGACACTTCTCTGAGCCTCCCTGGCCCTCTGCATTG |
| | | ACTCCAGATATTTAAGAAATAAATGTTGCTGAGAACTCCTTTTCCTCCTTCCAATGAGTGGTGATCATAGCCATTCATTG |
| | | TTTAACAAACATTTAGTGACTGGGTGAATAGTTAGTGATGTGTTGCCCATTCAATAGGCCAGGTAGTATTCTACAACCTG |
| | | GGAGCCGAGCAGTGACCAAGACAGCGCAGATTCCTGCTCCCAAGGGGTATGTANGGAA |
| Phase-1 RCT-38 | | TNATGACATGATTACGAATTTAATACGACTCACTATAGGGAATTTGGCCCTCGAGGCCAAGAATTGGGCACGAGGGTCC |
| | | TTCCCTATCAAAGCCAGATGCTTGAGAAGCCATGAAAGAGACCTCTGAAGTGACAGAAAGGAGGAAACAGCCTCAAGC |
| | | CCCATCTGGAATCTTCCTGGCTGCTGTCCTCAGCCGGTTCTTCTGGCTGTTGAGCATCGATGAGCTGTCGTCCCTTCCA |
| | | ATTGAGTGACATATCACTCCTGAGTATGCCCACTAGATGCGGTGGAGATGCAGAGGCATCCGGACCCCACGGCCCAC |
| | | CCCCTCCCCTCACACACTTACTCTCTGCCTAGTAATGCCACAGAGCTTCCATCCCCATGCAAAGGTCATCAGGCATGGC |
| | | TATCAGTTGGCTCTCAGGGTGGATTTGACATTCTCAGATGATTAGAAGTTGGCAAGAAGCAACCTTGGTGAATAACTCT |
| | | GGTGTCTAAACTCTGTACTTGAGTTTACAGTCTCAGTAGAGGAGACGCCAAAGCTGTTGCGAGTGACGGCAGGATTAT |
| | | TGAACAGTCATGATGCTTGGCTTTCAAAGGCGATTATCGCTTTAAGGCTTANAATTAGTAAGGCATCTTTATAACCAGGC |
| | | ATAGGTAGATCATAAACTACTGGAGGGCCAAAGGACCTAGAACGNGCTTCTTACCTTNCTCTCTAGNTACATTACGACA |
| | | AACATAATCNCCANNCTCAGGGGAAAACTTGCTGATTCAAGTA |
| Phase-1 RCT-39 | | TCTATGACATGATTACGAATTTAATACGACTCACTATAGGGAATTTGGCCCTCGAGGCCAAGAATTCGGCACGAGGGTA |
| | | AAACTGGCCAGGCTCATGTACAAGAAAAGACATGGTCCTCTATGGTTGTCTGAAACTCCTGGGGAGCCCTGGAAACCT |
| | | TGTAGAGGGCACTGGGGACCCTCATTATATACAGAAGTCACTGATGTGGACAAAGCTGGATACAGCTATGACCAGGCT |
| | | GGAGGGACAAGAAGCAAAGGGGTAGGTAAAAGAGCTCATGGTGTCAACTGCAGACAAGCCAAGTTGTGAATCCTGGT |
| | | CAGCACACCCAGAGACTTAGTCTAGAAATCCCTCCAGGATGCCTGGATACCTGTGCTCCCACTGACCTCAGATGAGGG |
| | | CCTGCTGTGGGACTGTGGTCCTTGGAAATCACTACCCTCTTGACGACCCAGGCACAACGGCATTACGTCATTCTGTTCT |
| | | CATTCATATTGTTTGCTCATGGTCAAGTTTGCCCATGGCTTTGGTAGGTGTCTTGAGCATTGGCTTGTTTGGGGAATGG |
| | | GGGGAGCGTTGGGAGCAGAGTCCATGAACAATTTTGTCCCTCAGACTGTCTTCATTTTTGGATGAGAGTGAATAACTCT |
| | | TTCCACATGGCCCTTTTGTGAGGCACACGTCTCCCCTGAA |
| Phase-1 RCT-40 | | NTATGACATGATTACGAATTTAATACGACTCACTATAGGGAATTTGGCCCTCGAGGCCAAGAATTCGGCACGAGGAAAG |
| | | ATCCAGTCACTGGGTTAGACTACTGGATTGTCAAGAACAGCTGGGGCTCTCAATGGGGTGAGAGTGGCTACTTCCGGA |
| | | TCCGCAGAGGAACTGATGAATGTGCAATTGAGAGTATAGCCATGGCAGCCATACCGATTCCTAAATTGTAGGACCTAG |
| | | CTCCCAGTGTCCCATACAGCTTTTTATTATTCACAGGGTGATTTAGTCACAGGCTGGAGACTTTTACAAAGCAATATCAG |
| | | AAGCTTACCACTAGGTACCCTTAAAGAATTTTGCCCTTAAGTTTAAAACAATCCTTGATTTTTTTCTTTTAATATCCTCCCT |
| | | ATCAATCACCGAACTACTTTTCTTTTTAAAGTACTTGGTTAAGTAATACTTTTCTGAGGATTGGTTAGATATTGTCAAATAT |
| | | TTTTGCTGGTCACCTAAAATGCAGCCAGATGTTTCATTGTTAAAAATCTATATAAAAGTGCAAGCTCCTTTTTTAAATTAC |
| | | ATAAATCCCATGAATACATGGCCAAAATAGTTATTTTTTAAAGACTTTAAAATAAATGATTTAATCGATGCTATAGAATTCC |
| | | TATGTGTGTTAC |
| Phase-1 RCT-48 | | TGACATGATTACGAATTTAATACGACTCACTATAGGGAATTTGGCGCTCGAGGCCAAGAATTCGGCAGGAGGCCTGGA |
| | | GCGAGCTAGCTCTCAATAGCTGGAGGCGGGAGGGGCTGGCACAATAGGAGCAAGGGCCAAGCCGGGAGCCGAAGG |
| | | CGGATTGGGCCAGAGCTTACCACATTATGGCTGAAGAGAGAAGCAGACAGATTCTCACACCGGCACAGGACTCCTTGA |
| | | CATGGGACTCCCTTGCCATGGGTCCCCCTTTTCCCTGGTTGGTCCCCTTCTGTCTCCCCCTCCTTCCCATCTTCTATCA |
| | | TTTTTTAAATCTCTTCTCCCACCATGAAATTAATTTGTTTCATATTTTTGGCTGAGTAGAGTTGAGAGGGCTCTGCCCTCC |
| | | CCTTCTCCCACTTCATCACCAGCCCAACTGCCCTTCCCTCTTTTGGTCTTCATGAATATATTTATATGGACAGAATTAAG |
| | | AAAAATAAATTGATTTCCCCATTCGTTCAATTCCCCCACCTTGTCTCCCCAAACCCCACAGAGTTAAAACTTGGGACTCC |
| | | CCCGACCCCTGATCCAACCCCAGAACACTTGTATATTGTTTGGTTGAGGTTTGTGCCACCACAGTAACAGACACAGTAT |
| | | TTAATTGCACATACAGATGGTTGCTGGGGTCACTGTAAAATTTTATTTAATCTGGGTT |
| Phase-1 RCT-49 | | TTATCCATGATTACGAATTTAATACGACTCACTATAGGGAATTTGGCCCTCGAGGCCAAGAATTCGGCACGAGGGTGAG |
| | | GTGTTCTCGTGGTTACGATAGGTCTCTTCCCTGTGATATTCATTTGCAGATGGCTGGACTGATCAAGCAGTACAGAATG |
| | | GAGGTCGGAGGGAGAGAAGGTCCTCCAGGGAGATGAGAAATCGCCGAGCACCTTAAGTCTCAAGGTTTGCTGACGGC |
| | | CAAGACCAGGCTTTGAATGAATGGTGAACTCAGAGGGGAGCGCGTTGGCCTGAGGAACCCACGGATGCCAGTGTTGG |
| | | TCTATTCTTGCTTTCAGGTACCCGTTGGAACACAGAATAGCAGTCTAGTCCTGCTGCCACCCCCACAAGGCTGGGCAT |
| | | GGTTCAAAGGCATGCAGGATGCAAAGAAGAGTCAGCTTTGGCTGGGGAGGAGTGGTTTGGTGTACACTGCTACTGAA |
| | | ATAGAAACTTTTGGCCTTCTGTCTGTAGAAATAAAAATCTGACTTGGTGATGTTTTTAAAAAAAAAAAAAAAAAAAAAACA |
| | | TTGCGGCCGCAAGCTTATTCCCTTTAGTGAGGGTTAATTTTACTTGGCACTGGCCGTCGTTTTACAACGTCGTGACTGG |
| | | GNAAAACCTGGCGTTNCCCAACTTAATCCCTTGCACACATTCCCCTTTCGCCAGT |
| Phase-1 RCT-50 | | TATGACATGATTACGAATTTAATACGACTCACTATAGGGAATTTGGCCCTGGAGGCCAAGAATTCGGCACGAGGGCAAT |
| | | CATGGCTCCGGGTTGGCCGCGGCCTCTGCCGCAGCTCCTCGTGTTGGGATTCGGGTTGGTGTTGATACGCGCCACGG |
| | | CCGGGGAGCAAGCACCAGGCAACGCCCCATGCTCAAGCGGCAGCTCCTGGAGCGCGGACCTCGACAAGTGCATGGA |
| | | CTGCGCTTCTTGTCCAGCGCGACCACACAGCGACTTCTGCCTGGGATGCGCAGCAGCACCTCCTGCCCACTTCAGGA |
| | | TGCTATGGCCCATTCTGGGAGGCGCTCTTAGTCTGGCCCTGGTTTTGGCGCTGGTTTCTGGTTTCCTGGTCTGGAGAC |
| | | GATGCCGCCGGAGAGAAAAGTTTACTACCCCCATAGAGGAGACTGGTGGAGAAGCTGCCCAGGTGTGGCACTGATCC |
| | | AGTGAGGAGCACCCGCGCTGGTGCCCATTCATCGTCCATTCATTCATTCTGGAGCCAGCCTGGCTTTCCAGAGACAAG |
| | | CCGCGGCAGACTCTTCCAACCACAAGGGGGTGGGGCGAGGTGGTGATTCACCTCCAAGGACTGGGCTTANGGTTCAG |
| | | GGGANCCTTCCAGGGTGTCTAATTGCCCTGTCTCTGGNTCTGGGGCAGACAGANANCCTCAAGCTAGGTCACAA |
| Phase-1 RCT-55 | | TCTCTATGACATGATTACGAATTTAATACGACTCACTATAGGGAATTTGGCCCTCGAGGCCAAGAATTCGGCACGAGGG |
| | | TTCCGGAATGACATTGCTGCCAAGTACAAGGAGCTGGGCTTCCAGGGCTGAGCCATGGGCTCCCACTGTCCAGCCCA |
| | | GCAAGCTGGGACCCAATGTTGTGTAGAAGGTAGAGTGTGCTGTGCCCTAGGTTAGCAGAGAACAGAAGAGGGGAGCA |
| | | TAGTGTGGCATCTACCCACACCCCTGGGGACAGAGCTCTGGGCAGCATTGTCCTGGAACCCAGAGGTGCAAAGTGGC |
| | | CTCTGCTTCCTCAGCTCTGCTGGGTCATGCTCAGGTCTCCTGTCACCTAAGTCCCAACCCACTTTCCTCCTGGTTTTGG |
| | | GAAAAATCTCTTTTCCACTGTCACATTTGACCCCAAATGCAAGTCACCAGCTAGCAGACACTGACCTTTGAAGGAGACA |
| | | GAGGGTTACTTANAGGGAGTGGAGGGTGGAAAGTGGGCAGGTGAGCATCGGAAGTCTCCAGCTGCTTAGCTTCCCCC |
| | | TGACCACCTGGTTTCAATAAAACATCCTGCAACTCCTCNTTTNGGCNCNNNNNTNTCCACNCCNNNAAAAAATANAANN |
| | | GTTGTGCGGCCNCAAGCTTTTCCCTTTAANGGAGGGTTAATTTTACCTTGCCTGGCCGNCGTTTNACACC |
| Phase-1 RCT-58 | | TCTATGACATGATTACGAATTTAATACGACTCACTATAGGGAATTTGGCGCTCGAGGCCAAGAATTCGGCACGAGGCCT |
| | | GTTCAGAATCACTACTTGAAGCTTCAGATCCCTTCTCTACAATGAACCTCAGCACTGCAAACCACCACATACCCCTAAAT |
| | | GATGGTAACAGCATTCCGATCATCGGGCTTGGAACCTACTCAGACCCTAGACCGGTACCCGGCAAGACCTTTATAGCA |
| | | GTGAAGACAGCTATTGACGAGGGGTACCGGCATATTGATGGGGCCTACGTCTACCGAAATGAACATGAAGTTGGTGAG |
| | | GCCATCAGAGAAAAGGTGGCAGAAGGGAAGGTAAAGAGGGAAGAGATTTTCTACTGTGGAAAGTTATGGAGTACAGA |
| | | CCATGATCCAGAGATGGTCCGCCCAGCCCTGGAAAGGACCCTGCAGACCCTCAAGCTAGATTACATAGACCTTTATAT |
| | | CATTGAGATGCCCATGGCCTTTAAGGCACTGGAAGCTCAAAGACGCTGGCTTGGTGAAATCCCTTGGGGTGTCTAATT |
| | | TTAACCGCAGGCAGCTGGAGGTCATCTTGAACAAGCCAGGACTCAAGTACAAGCCTGTCACCAACCAGGTGGAGTGC |
| | | CACCCGTATTTCACCCAGACAAAACTCTGGCAGACCTCTCANGAGAATCANGCTCCTGTCAGCAGAA |
| Phase-1 RCT-59 | | CCNNNTTCTATGACATGATTACGAATTAATACGACTCACTATAGGGAATTTGGCCCTCGAGGCCAAGAATTCGGCACG |
| | | AGGCAAGATGGTGCCTCGGGGGCTGAGGGAGGTCACAGGAACTGAGCAGTGACTGGTCCTTTCCCAGTATTGAATAC |
| | | TGAGCCCCTGTGGGTGTGGAAGCACTTAGTGGGTCTGGCCCCAACCCCAAACACCCCTGTTTCTGTAACACCCTGAGC |
| | | TGGACTGTTTATCTTTAGCCGGGAGAACATGTATTTTGGTCCCTTCCCTGTCTCCGCTCAGATTGTAACCTCCCACGT |
| | | GTGGGGATCACACCCTGCACTGTCCCGAATCTTTACACCCTATCCCAAAGCTGGTGCTCAATAAATACTTCTAGATGAT |
| | | TAAAAAAAAAAAAAAAAAAAAAAAAAAATGGAAGCGGCGGCAAGCTTATTCCCTTAGTGAGGGTTAATTTTACCTTGG |
| | | CACTGGCCGCCGTTTTACAACGTCGTGACTGGGAAAACCCTGGCGGTACCCAACTTANTCGCCTTGCAGCACATCCCC |
| | | CTTTCGCCAGGTGGGCGTAATACCGAAAAGGCCCGCACCGATCGCCCTTTCCCAANAATTGCNCAACCTGANTGNCAA |
| | | ATGGGACCCCCCCCTGTAACGGCNNATTTAANCGCGGGC |
| Phase-1 RCT-61 | | CNNNNNNNAATNNNNNNNNTNCTATGACATGATTACGAATTTAATACGACTCACTATAGGGAATTTGGCCCTCGAGGCC |
| | | AAGAATTCGGCACGAGGTGCTTTATTGTACAAAGCAGCGTCTCTGTGTTTTGATCCCCCATNGTTTGAATTCTCGNCTTT |
| | | AGCTGGTAGAGTCTCANATATCCCTGACACGGGGCATTCTGTGTGCGGTGAGGGGTTCCCGAGAGGNTCCGGAGCCC |
| | | TGCGTGAAAGCAAGCTCTCAGNGTACGGGNGATTGTGCAAAGNTCAGCACGGCANAGCATCCTGTGGTCCAGGTCTC |
| | | ACTTCTGCTCTTTATTGAAGACAAACATTCNCCANTAAAGAAAAAAAAAAGAGAAAGATGTTTTGAGANAAACTAATTTTC |
| | | TTTGACAAGAGTATTACTTAATTTTTTGGCCTATTAAGGTTCCCCTAGTTAGNACTCNATTCCCTGTGCTAATCGTTCAAC |
| | | TGTATATTGTTAAAACACACTGCTGATTCTGTACCAAACTGGTCTCAAAANAAAAAGAAAAAAAAAAAAACNTCTAGCGG |
| | | CCGCAAGCTTATTCCNTTAAGGGAGGGTTAATTTTANCTTGGCCCTGNCCGNCGTTTTACAACGTCGNGACTGGGAAA |
| | | ACCCTGNCGTNACCCCACTTAATCGCCTTGCAGCACATCCCCC |
| Phase-1 RCT-65 | | CTCTGACATGATTACGAATTTAATACGACTCACTATAGGGAATTTGGCCCTCGAGGCCAAGAATTCGGCACGAGGATAG |
| | | AAAGTGAAGGGCTGAGGGTTGGGAATTCATTTTCAAATGATGAGATGATTGGCTGGGTTCTCTGTCTACCTTACCTGCT |
| | | GTGAGGGGCTCTCCTAGGGCCGCTCTTACATTTACAGCTGCCTTCTTTGTGACAGACTTTTTCTTTTCTCTTTTTTGTGG |
| | | TGCTTGGGATGGGACCCAGGGCTTTGTGCAGGCCTGGTAAGGCTCTGTCCCTGGGCTGCACCGAGCGCCACCTCTCT |
| | | ATGGGTGACCGTATGTAATCCTGACAAAGACCAGCATGTGAGCCACTCAGAAGTTAGCCAGGATCGGGGGAGTTTGCC |
| | | GAAGTGCTACGTACACTTTACTGTGGAGAGGGGTGAAGAGTGTGCCACCTTAAACTCCGTGACGCTCAGGCTGCCAC |
| | | GGGCTCCTGTCCCACCTCCATTTGCATCAGACCGAGTGAGGGGTCCTTCACTTCTCTGGGGATAATAGTGGTTGTCAT |
| | | AGTGAGTGTGTATTTTCCAGTTTTGCTTGTCAGTCCCCCCCAAAGAAGAAGGAAGCCNCTGATTTCTTGCTGTTTGGTTT |
| | | GAGACAAGGATGTTACTCTGGANCCCTGGGTTGACTGGAAGCGTCATAAATANACCACGCTGGTCTCAAACTTATGGC |
| | | AC |
| Phase-1 RCT-66 | | TCTATGACATGATTACGAATTTAATACGACTCACTATAGGGAATTTGGCCCTCGAGGCCAAGAATTCGGCACGAGGAGC |
| | | ACTTTGCTTTAATCCACAAGAGGCAGAGGCAAACACACAACTGAGATCCAAGCCAGCCTGGGCTACATAGTGAGACCT |
| | | TATACATCTAAAAACCAAAAGAAATCCCATAATCCAGGCTATTTGGGGGAACTTGGAAGAATACTTAGAAATGGCCGTG |
| | | GATTTGTCACACGGGTGACCTAAAAAGACAGTGGGGACCTTGCTTGGGTTCCTGGGGTGCCCACTGTCCAGCCACTA |
| | | GGACACTTGTAAGGAGCTCTGTGCTATGGCACCGGGCCCGGTCTTCACTGTATATTCAAGGATAGCACACATATATATT |
| | | AACTCTATTTATTTTGCTGACCCTGACTGCTGCACTTGTCGGTGACGTTTCTCCAGGTTAGGCTGGGGTGATACTCTGG |
| | | GATGCTCTGCGTACAAAGATTATTTGAATGAACTGATCTCAGGCCTGTCCTGTGTGTGTTGCTTTGGGGGTGGGAACTG |
| | | CTTTGAAACCCTTGTTCGGATGTTTTTATAGGCTGAGAATGTCTTGCTGTGATGGATTAAATTCCTTTTTGAAAACTTAAA |
| | | ANNNNNNACNNCNNNNNCNNNNAAAN |
| Phase-1 RCT-68 | | TATTACATGATTACGAATTTAATACGACTCACTATAGGGAATTTGGCCCTCGAGGCCAAGAATTCGGCACGAGGTTGAT |
| | | AGAATCAGTAAGTTTCTAAGGGAAAGGAAATTGATATTTTGCAGACCAATTTGTTCTAACCAGCATCCCAACTCTAGCTC |
| | | TGTCAGCCACGTTACCGCATCCACCCTTTACTGCATGCTCAGGTCGCTGCAGTCTGGTTCTCCTGGGAGATTTTCATCA |
| | | TGTAGCTATTGGATACAATTATGAAAACCAACTGTTGAACATATTTGGAGTAGCTGTTTCTTTCCTAGAGAACCAAAGCT |
| | | GTTTCAGCTGGTAGAACAGGTTGAAGCCCGCCTGCATTAGCTGTGCTTTCCGTTATGTTTAGAGGGATGCACAGGCAC |
| | | GACATCATTCCAGGAAGGAAAATTGTGGTTAAGAATTTCCAGTAAGATCATACTTAATAGCTGAGATTTTTAAGGCATTT |
| | | TTATGTTTTCAATGACATAACAAAAGTCAACTCATGCTTATAGCAAGGTGAACAAACCCTAGGTTCTCTTCAGACGTTAA |
| | | TTGATAGTATTTAATGTACGTTGAAAGTTAACCTCCATTAATGACATCTTTTCTTTGTGGTANGGCCTACCTTCTGCTTTC |
| | | CTGGAAAAGATGAAATATACATCATTTGAACCTATTTTGAGTTTTCTGTTGGGGGGGGGGGGGGGTT |
| Phase-1 RCT-70 | | TATGACATGATTACGAATTTAATACGACTCACTATAGGGAATTTGGCCCTCGAGGCCAAGAATTGGGCACGAGGAGCG |
| | | GGACTACTTAGTCAAACCGTTATTACGGTTTGATGTTATGGGACAACGTTAAATTCCTTGAGGGGCTTCAGAGTTGCCA |
| | | TTTGCAGCCTGTCAGTTAAGTTCAGCATTAGAGTTTGATTCTGATCTTTAACCCTGTTCAGGGCGGTCTGCCCTCACAAT |
| | | CCAGATCAGCACACAGCGGAAGTATCTGACTTTCCAATGAATTGTAGTTTTTTATTGTATTTTAATTGGGCTATCTGCTCT |
| | | GCACCCAGCCCCACTCTAAAGGTAAAAGCTACGATCTGTGGAGTACACTGGACCCTGGTAGGTCCAAAGCATTAGGTG |
| | | GGAGCTCAGAAAATATTGTCAAGTAGGTGAACTGTATTTAAATGAAACAAAAAGGCATCGCAACTCTACATTAATGAGA |
| | | CAGCAGGTGCCAATACCTCTTAATCTATTCAAGACACCAAAATTAATCATTTTGCAGCTACTAAGTCATGTGCAATTTTA |
| | | GTAAGTGTTGAAAAGTGACCTTTTCACCTAAGGCATGAATCCCCTGGGCTGGGGATTGAACCCCAGACCTGCCTGGTA |
| | | TATGCTACATAATTGCTCAACCACAGNAGTACGCCTTCCATCCCCACNNATGNNGGNNNNGGGGTTTT |
| Phase-1 RCT-71 | | TATGACATGATTACGAATTTAATACGACTCACTATAGGGAATTTGGCCCTCGAGGCCAAGAAATTCGGCACGAGGCAAA |
| | | AAGAAGTCTAAAAGCCTATAAGAAACCTGAGCTGCTTGCCCGACTGCAGAAAGATGGTGTCAATCAAAAAGACAAGAA |
| | | CTCCTTGGGAGCAATTTTGCAACAGGTGGCCAATCTGAATCCTAAGGACCTCTCCTATACTTTAAAGGATTATGTATTTA |
| | | AGGAGCTCCAGCGGGACTGGCCAGGTTACAGTGAGACAGACAACAGACACTGGATTTGGTGCTCTCTAGAAAATTAA |
| | | ATCCATCTCAGAATGCTGGCACTAGCCGGCCAGAGTCTCCCATGTGTTCCAGCAAAGATGCTGCCTCATCTCCTCAGA |
| | | AACGACCTTTGGATTCAGATTTCATTGATCCTTTAACGAACAAAAAAGCTCGAATATCTCACCTGACAAACAGAGTCCCA |
| | | CCGACATTAAATGGCCATTTGAATCCCACCAATGAAAAATCTTGTGCAAGCCTTCTACCGCCCCCTGCAGCTGCTGCCA |
| | | TCCCCACCCCCTCACCACTGCCTTCAAACCACCTGCCTGTCTCCAATCCTACCCAGACTGTAAATTCCAACTCCAATTC |
| | | CCCTANCACTCCAGAAGGNCNGGGGGACTCAAGAACTACCTGTTGACAGTTTTAGTCCAAAGGGGNNGGGGGGGGG |
| | | N |
| Phase-1 RCT-72 | | TATGACATGATTACGAATTTAATACGACTCACTATAGGGAATTTGGCCCTCGAGGCCAAGAATTCGGCACGAGGCTCAG |
| | | ACACTGAGGATTGGAAATAAGGGCCTGGAGTAATACCCAGTCTTCAGTAACCATAGGCCCAGCCAGAGACCTGTGGTG |
| | | GTTCAGCCACAGCAGGGGCTGGGCAGAACCCCTCTGAGCTGCCAAAGCTTGTAAGTGCCAAGTAAGCCCCTTTCCT |
| | | CCCAGCATGCTCTGGCTGAAGGGGTTGGCCCTGCCCTGACACCTTCTCAGTGCCTCTCCCTATGCTTCCCCAATGATG |
| | | GTGCCCAGCCTGGGGCTGGGCCCACATATGCCAGTATGAAGGCCGCCTGGATGAAGAGGGCAGGCTCCCACTCCCT |
| | | TCATTTCCCCTAATGGGTGCTGGCCTCCCCACAAGTTTCAAGACTGACATTTCAAGGCAGCTCTCAGGACTGCCATTTT |
| | | TCTCTACACCTGTGGGTTTAGCTTTTATTTTTTAAGTCACAACTTTGATAAAAAGTGTTTCTATCTTACTGGTTGAATT |
| | | TAGCTTACTAATTCACACACCTGGAAGCAGCAACTTGTAGCATGTAACCAAGCCAGTTGGTTACTCTGGAAATGGGACA |
| | | GTATGATGCCATCCCATGTTGAACACTTGCGTCCNATNAAACAACGCTGGAATTC |
| Phase-1 RCT-73 | | TCTCTATGACATGATTACGAATTTAATACGACTCACTATAGGGAATTTGGCCCTCGAGGCCAAGAATTCGGCACGAGGG |
| | | GGAATTAGCAAAGGAAGCAAAAGGAAGGAATGTTCTATTAAGTGGAGTCCTCCTAAATCACTCACAGGATGAGCAGAA |
| | | GCTACNAGAGAGTTTAGTACAAAGTGCAGCCATCATAGCTGCCTTGGTTAAGGAAAGAAATTCTGCACTCGTCGGGCA |
| | | GCTCCTGGTGGCATGAAGAACACAGACAAAAATCATCTATAAGCTTCCAAAGATCCTTCCCTAAGGCCCCCGACCCCAC |
| | | TTGACTGCCTCGCCACCTCAATGTCTCTTCCTTGTGCATACAATATTTTCATTGTTTTCTTATCTTGCCACTTATTGGATG |
| | | TGACGCTTAGGTTGTGACAATTGGTTAATGTGTGTGTTCATGAACTTATGTATCCAATACAATTGGCTCCAACTACAAAA |
| | | GGATAAATAAATGGAAAATATTGCTTTCATGTAAAGAATATGTTCACTACTGCAGCAAGTGCAATTGACATGATAGGGTT |
| | | AGCCATTCCTTCAAGGGCCAAGAGTTTAGACAGCTTGGTGGTTATGGTAAGTTTATGCTATTAACATATTATTCACATT |
| | | ATAGTGGAGCTTATGGATANGCTTTCAGAATTATACCCNATTCCAAAATCATCGTCTTCCTATGT |
| Phase-1 RCT-75 | | TATGACATGATTACGAATTTAATACGACTCACTATAGGGAATTTGGCCCTCGAGGCCAAGAATTCGGCACGAGGGGAAA |
| | | GCCTTCCACAACTTCAACTACTTCCTGTTTTTCTACAACGTGGTGCTGGGCCTGGGTGCCTGCCTCTCCAGGCTGCTCA |
| | | TCAGCTGCCTCCTGGGCACATGGCTGATTGCACGCATCGACAGGACCATCATGCAAAGCGGCTATGAGGGTGCAGAC |
| | | ATGGGCTTCGGTGCATGGATCGGGATGCTCTTTGTGGACCATTACCACACCAACCCTGTGCTGGTCAGCTTTTGCCAC |
| | | ATTCTGATCACAAGCCACAAGGATGGAAAGCTGCAGAAGACTGCGAAACATGGGTGCCCAAACCAGTCCGCAGGTCC |
| | | CCGATTCTCGGCCAGGGCCAGAACGAGGTGGTTCCTGCTGCAGACGCTGATCAACAACCCCAGGCTCGCCATGCTTA |
| | | GAAAATCAAAATCAGGGCATAGTTTCAGAGAGTTCACACAGATTCTGCTGACCTGCTCAGACTGCTGATTGGGCCACCA |
| | | GCTTCAGCTCCGAGGCTAGGCCATCATCCCCTTTCACAAGGGACTCAGGCTAACTATCAGCCATTCCCTACCATGGGA |
| | | CAGGATGACATGGGCACTGTTCTGCACATGATANACAACACTGATCACTTGTGCGATGGGCATGTAAAACCAATGGT |
| Phase-1 RCT-78 | | TATGACATGATTACGAATTTAATACGACTCACTATAGGGAATTTGGCCCTCGAGGCCAAGAATTCGGCACGAGGATTTT |
| | | GTAATTTGTGCATGAAATTCATGTTATTTCCTGGGGAGAGAAGAGGGCCAACTGAGTGGGTGGGGGGCACGTGTGGG |
| | | AAGCAGGCAGAGGGCTCCTTCCCTGCAGGGGTGGGGAGGGGTCGCAGAGGGCATGCTACCCAGCTCACCTGAAGAG |
| | | GGCTCTTGAACCAGCCGTTTTGGGTTGTGTTAAATGTGGGAACTTTCCTCCATTAATGTACAATCTTGAACTAACTGCTA |
| | | ATAAAATGGGATTCTGTTTGTAAAAAAAAAAAAAAAAAAAAAAAAAAAAAAAAAAAAAATCTCGCGGCCGCAAGCTTAT |
| | | TCCCTTTAGNGAGGGTTAATTTTAGCTTGGCACTGGCCGTCGTTTTACAACGTCGTGACTGGGAAAACCCTGGCGTTAC |
| | | CCAACTTAATCGCCTTGCAGCACATCCCCCTTTCGCCAGCTGGCGTAATAGCGAAAAGGCCCGCACCGATCGCCCTTC |
| | | CCAACAGTTGCGCAACCTGAATGGCGAATGGGACCCCCCTGTAACGGCGCATTAAGCGCGGCGGGTGGGGTGGTTA |
| | | CCCNCAACGTGACCGCTACATTGCCAGCGCCCTAACNCCCCCTCCTTTTCCTTTCTTCCCTTCTTTNTCGCCCG |
| Phase-1 RCT-81 | | TTATGACATGATTACGAATTTAATACGACTCACTATAGGGAATTTGGCCCTCGAGGCCAAGAATTCGGCACGAGGGCAG |
| | | TTTGATGCTGTGTTCGCCGTAGAGCAGTAGTTCTCTACGTGTGGGCCGTGACCCTTTTGGGAGCCACACCTCAGATAG |
| | | TTACCTTACAATTCATAACGGTAGCAAAATTAGTTATGAAGTAGCAACATAGAAAATTTTATGGTTGGGGTCACCCAAGG |
| | | ATAAAGAACTGTATTAAAGGGTCATAGTGTTTGGAAGGTTGAGGACCAGTGAATTAAGTGGAATTTTTACACTAAGAGA |
| | | GTGGTAGAACCAGGATGCAGGCAGTGTACACATTGTTGGGGAGCTGCAGGCTCAGTTCTCAGGACAGGACCTGGATG |
| | | CGGGCAGAGTGCAGTGTTGGGGAGCTGTGGGTAACAGTCACTTAAGTGGGTTGATCAAAACGTGAACACTACCTCCAT |
| | | GCCAGACAAGGGCCTTGCTGATGATACAGGGACGGCAAGATTGGAACACGGCAACAGCTCTGAAGCCTGTGGGCCCTG |
| | | ACCAGTGAGCCCCGCCTTCTCACAGGCTGCCAGCACCCANGCAGCTGTNCCGCCTCTGTCTGCACCTGCTCTAGCCG |
| | | GCCACACATCTTCATCANTTGGGATTGGGATTGANCCTGANCTGCCGGTGTCTGTAATTATCTAATTTCGCTGGNGGNA |
| | | TTCCACCANCCTCTG |
| Phase-1 RCT-82 | | TATGACATGATTACGAATTTAATACGACTCACTATAGGGAATTTGGCCCTCGAGGGCAAGAATTCGGCACGAGGGGAA |
| | | GAAGAAGCTGACAACAAAGACTTCAAAGAAGATGGAGAGAAAGGGGAGCCTGTGAGCACTGTCTAGATCTGCCCTCTG |
| | | TGAGATCATAATTTGGTAACACGTACCTTCCTGTTGTAATGTTAATAGAGATAAATATTTTTATCAGATATTTTATAAACAG |
| | | TGCTTTTCTTTAGCATCATTCAATCTCAGATCATCATCATACCAGTTATTAGTTGCAAAATACCTAACATGACACCTTTATA |
| | | CATTTTGTGATTATAGTAGTGCAAAATAGGAAATGTATACTTGCAACTTTGTGAATTTCATTGTGTGTAAGTTATGTATTA |
| | | AATCTTTGAGTTTCAACTTTATTCTTATACGTGGTAATTTGCAGAGTAAGAGAATTCCAAGAAAGAGTTTGGCACAA |
| | | CTTTGCTGTAGTTCTCTAGGTTGCTTATAAAGAAGGTCAACTATTTTCAGGTAATGCCAAGAGTTAGGATTGCCATTA |
| | | CCAAATATAACTGTATTAGTAGCTTGGAAAGAATATAAAATTTTAATTTCATTTTTGAAGAAATTTTGAATGTTTATTTCAG |
| | | GAGGGCAGTTTTGACTATATGTGTATGCACAAAGTTTTGCTGGGATTTATCCATAATAAAACC |
| Phase-1 RCT-83 | | TATGACATGATTACCAATTAATACGACTCACTATAGGGAATTTGGCCCTCGAGGCCAAGAAACGGCACGAGGTGAT |
| | | TATTGTGATGGAAATTCTCTTTGTACCACCTTAAATTAGTTTGTTAATCCAAAGTGGAGGAAGACAGCAATAAAATGAAT |
| | | CCAAGTTTAAAAATGGGAAGACATAGGGTAAGAGCTCTCTATTATAAAACTGATTGCATTGTGTTTCTTCTTACACATCA |
| | | GTATATTGCATAGCTCTCAATGAAAGATTGACGAACTTGCTAAGCTTACTTTTCACAAACTCAAAAAATCCTTTAAAGGG |
| | | CATAGGAAAAAAAATCATTCAACAATAATACCCTTTTGCTTCCTTAGAGCTATACTAAAGTATAGAATTTCAAAACAGGAC |
| | | ACCCTTAGATTTTCCACCTATTCGTAACATGGACGCTGGAACTCACAGGAGAGCTGTCTGTGGTTCCACAAACATCATA |
| | | TTTTTTGGTGAGTGACTGTGGTTGTGATTACAGAGCAAGAAGTAAGGCTCTCCAGGCAGTGATGGCTACTTCAGGTATG |
| | | ACTACCATGAAAGACTATGTGGAATTTCTTTTACCTTTGGAAATGTTTGAAAAGCTAGTAAAAAGAATTAGGGAATTTAAA |
| | | ATTTCANGGGAAAAATATG |
| Phase-1 RCT-87 | | TTATGACATGATTACGAATTTAATACGACTCACTATAGGGAATTTGGCCCTCGAGGCCAAGAATTCGGCACGAGGCCAA |
| | | ACTGCACAGAGGGGAAATCAAGCTGAATTCTGAGCTGGACTTAGATGACTCCATTAGAGAAGTTTGCCTTCTCCAAC |
| | | GCTCTTTGCCTCTCAGTGAAACTGGCTATTTGGGAAGCAACACTGGACAAATTTATTGAGTCTATTCAGTCCATCCCAG |
| | | AGGCTTTAAAAGCTGGGAAGAAAGTCAAACTGTCTCATAAAGAAGTTATGCAGAAAATGGGTGAGCTCTTTGCCCTCAG |
| | | GCATCGAATAAACCTGAGCTCAGACTTCTTGATCACCCCTGACTTCTACTGGGACAGAGCGAACCTGGAGGAGCTTTA |
| | | CGACAAGACCTGCCAGTTCCTCAGCATCACTCGAAGAGTTAAGGTCATGAATGAAAAACTGGAACACTGCATGGAACTA |
| | | ACAGACCTAATGCGCAATCACCTCAATGAGAAGCGAGCGCTCCGCCTGGAGTGGATGATCGTCATCCTCATCAGCATT |
| | | GAGGTAATGTTCGAGCTGGGGAGAGTCTTCTTCTGATCTTGTGATCATGAAAGCACCACTGGAAGAGAGTAGTCAAGT |
| | | TCTGCAACCAAAAAACCAGCGTTCCT |
| Phase-1 RCT-88 | | TTATGACATGATTACGAATTTAATACGACTCACTATAGGGAATTTGGCCCTCGAGGCCAAGAATTCGGCACGAGGCTCC |
| | | TGGGTTCCCAGTCTGAGCTTTCCAGGTATTGATGCTTCACAGGCACTGCTACATACTGCTGAGGGAAACATGTCCTGC |
| | | GTGACTTTACTGGGAAAATTCCTAGCAAAAATTTTGTGCCTGATTTCCTCTGGACGTGTGCTGAATAGACTTCCACAAGA |
| | | GTTGTGCACACACATTCCTGAATCCTTCTCTGCTTCCTGCCCTTGGAATCTAACTGCCTCAGCCTAGAGGAATTCTAAT |
| | | CATTCCCTGCTCACCCAAGGACCGCTGGCCCTGTTTATCTTGTCATTACCTAGAAATTCCTTCTTATCAGAAGGCGCAT |
| | | CTCTTTCATTGTCCCTTTTCCCCTCGTCCTTCACTACTTATAGAGGAATATTTAAAAGAAACACAGAGGGGATGAATTCA |
| | | CTCGAAAATTGTTGTATGAAATTCTCAAGGAGTTAAAACCATGTTTCAAAAGAACAAAATGTGGACAGCATGCTGCCATT |
| | | TGCTTAAGGAAGTGAGATAGAGAAATGCACCGAAGGATTTCTGGAGGCGTCCAGGCTGCTTCTTGCTACTTCAGCAGA |
| | | CTGGTGCCCACTGGTGGGGCCTCTCCGGGGTTCTTCTGGACAGAA |
| Phase-1 RCT-89 | | TTATGACATGATTACGAATTTAATACGACTCACTATAGGGAATTTGGCCCTCGAGGCCAAGAATTCGGCACGAGGTGTG |
| | | TTCCTCTGAAATTCTGGACTGATTTGGACAACTGAATCTGGAGCGTGTGGGATACACAGGGGAGTTGATTTTTTATAAG |
| | | CACATGAAACAGACAGTAGATTTCACACTTTTCAGGACCATAGTGTTTGTCATGCTTTAGTTATATGTGAGTGAAGTGGC |
| | | AGAGGAGGTCATGAGGCAGTTGGATTCTGACTGTACTGGAACTGACCTTAAATGAGTAGAGAACATTCTTGGCCAGAA |
| | | CGTGGCACTGTGTAGAGGCAGCCCCAACACTACCTCTTAATTGGTTTATTACATGTAACTTTCCATATTTTCCCTCCCTT |
| | | TTAAAGTCAGCAGTATTTTCGTTTTAAATGTCATCTTAGAGCCGCTCTACAAAATGTTCCAACAGACACCACTAAAACTA |
| | | GACCATTCCGAATTCATGTTCTTTCGAGTTTTATCCATTATCCTTAAAAGTAAGTGCTGTTACACCCACATTCCTGATTGT |
| | | ATGATCTTCTAGCTCTGTACCAATACAGGGTTTCCTGTAGTCCAGACTGGCCTCATATTCACTGCATAGCTGAAGATGA |
| | | CCTTGAACTTCTGATCTT |
| Phase-1 RCT-92 | | TATGACATGATTACGAATTTAATACGACTCACTATAGGGAATTTGGCCCTCGAGGCCAAGAATTCGGCACGAGGTGCCT |
| | | ACCCGGTCTCATTGTTCATGACTGCATATCATTAGCGCGCTTCAAAGGTGTTCATATTTTCGAAGATCTGGGGGGTTTTT |
| | | TTCTATATCGCAGGATATTTTTGTACATGCTTAGAGACCTCATTCAGTTGATAATCCCAACATTGTTTGTCATCCTTAAAT |
| | | CATGAGAGTAAACCCAAGTATGACAAATTAAAAGAAAACTCTAGTCTTTCTAAATTTGTCTTGTCTAAAAAATATGTTCCC |
| | | ATAGACTGCCAGCATTGGTCTCACCTAAGGACGAACCACTCCTCCTCATTCCTTGTCTTCAACTCATGCATTTGTAAATG |
| | | ATGCTGGCAACCTACATGAACAGACAACATTGTCTCCTTGCCTCTGGACAGCCTTACCAGCTGGTCTCATCTTCCTGCA |
| | | TGGCCACACCCCTAGTGATGGAACTCAGGTANCATAGCACAACGTGAAGTTGTAGTCTGTTGAGCCTCCCATACCAAT |
| | | GAGAAAAGAAGCTTTGGAATTCCAGTTTTCTGAGATTCTGGTAGTACCTTCATATTTCATGTTGTAGACATTTGAAACTG |
| | | NGGTAAACTTTATTTTT |
| Preproalbumin | V01222 | TATGACATGATTACGAATTTAATACGACTCACTATAGGGAATTTGGCCCTCGAGGCCAAGAATTCGGCACGAGGCCCCA |
| | | CTAGCCTCTGGCACAATGAAGTGGGTAACCTTTCTCCTCCTCCTCTTCATCTCCGGTTCTGCCTTTTCCAGGGGTGTGT |
| | | TTCGCCGAGAAGCACACAAGAGTGAGATCGCCCATCGGTTTAAGGACTTAGGAGAACAGCATTTCAAAGGCCTAGTCC |
| | | TGATTGCCTTTTCCCAGTATCTCCAGAAATGCCCATATGAAGAGCATATCAAATTGGTGCAGGAAGTAACAGACTTTGC |
| | | AAAAACATGTGTCGCTGATGAGAATGCCGAAAACTGTGACAAGTGCAAGTCCATTCACACTCTCTTCGGAGACAAGTTATGCGCC |
| | | ATTCCAAAGCTTCGTGACAACTACGGTGAACTGGCTGACTGCTGTGCAAAACAAGAGCCCGAAAGAAACGAGTGTTTC |
| | | CTGCAGCACAAGGATGACAACCCCAACCTGCCACCCTTCCAGAGGCCGGAGGCTGAGGCCATGTGCACCTCCTTCCA |
| | | GGAGAACCCTACCAGCTTTCTGGGACACTATTGCATGAAGTTGCCAGGAGACATCCTTATTTCTATGCCCCAGAACTC |
| | | CTTTACTATGCTGAGAAATACAATGAGGTTCTGACCCAGTGCTGCCAGAGTCTGACAA |
| Proliferating cell nuclear antigen gene | Y00047 | CNCAAGCTATGCATCAAGCTTGGTACGGAGCTCGGATCCACTTAGAAACGGCCCGCCAGTGTGCTGGAATTCGCCCTT |
| | | CGCGGGATCCGGGGCTGAAGATAATGCGTGATACCTTAGCANTAGTATTTGAAGCACCAAATCAAGAGAAAGTTTCAG |
| | | ACTATGAGATGAAGTTAATGGACTTAGACGTTGAGCAACTTGGAATCCCAGAACAGGAGTACAGCTGCGTAGTAAAGAT |
| | | GCCATCTGGTGAATTTGCACGTATATGCCGGGACCTTAGCCATATTGGAGATGCTGTGGTGACCTCCTGTGCAAAGGA |
| | | CGGGGTGAAGTTTTCTGCGAGTGGGGAGCTTGGCAATGGGAACATTAAGTTGTCCCAGACAAGCAATGTTGATAAAGA |
| | | AGAGGAAGCTGTGTCCATAGAGATGAATGAGCCAGTTCAGCTAACTTTTGCTCTGAGGTACCTGAACTTTTTCACAAAA |
| | | GCCACTCCACTGTCTCCTGCAGTAACACTCAGTATGTCTGCAGATGTACCCCTTGTTGTAGAGTATAAAATTGCTGACA |
| | | TGGGACACTTAAGTATTATTTGGCTCCCAAGATCGAAGATAAGCTTGGCCAAGGGCGAATTCTGCAGATATCCATCAC |
| | | ACTGGCGGCCGCTCGAGCATGCATCTAGAGGGCCGATTC |
| Protein kinase C alpha | X07286 | AATTGGCCCTCTAGATGCATGCTCGAGCGGCCGCCAGTGTGATGGATATCTGCAGAATTCGCCCTTATCGCGGGATCC |
| | | CCAAGGAAGCTGTCTCCATCTGCAAAGGGCTTATGACCAAACACCCTGCCAAGCGGCTGGGCTGCGGGCCCGAGGG |
| | | GGAGAGGGATGTCAGAGAGCATGCCTTCTTTAGGAGGATCGACTGGGAGAAGTTGGAGAACAGGGAGATCCAACCGC |
| | | CATTCAAGCCCAAAGTGTGCGGCAAAGGAGCAGAAAACTTTGACAAGTTCTTCACACGAGGGCAGCCTGTCTTAACAC |
| | | CACCAGATCAGCTGGTCATCGCTAACATAGACCAGTCTGATTTTGAAGGGTTCTCGTATGTCAACCCCCAGTTTGTGCA |
| | | CCCAATCTTGCAAAGTGCAGTATGAAACTCAGAAACAAAAGATCTAATGCCTCCCTAGCCCCCAATCTCCCCAGCAGTG |
| | | GGAAGTGATTCTTAACCATAAAATTTTAAGGCTATAGCCTTGTATTTTGTTCCACACAGAGGCCTGAAAATTCTGGGGAT |
| | | ATTAGTCCATAAGTGATCAACTTTCTTCCCCAAAGCTTGGCCAAAAAGGCGAATTCCAGCACACTTGGCGGCCGTTACT |
| | | AGTGGATCCGAGCTCGGTACCCAAGCTTGATGCATAGCTTTGAGTATTCTATAGTGGCACCTAAATAGCTTGG |
| Protein O-mannosyltransferase 1 (Pomt1) | NM_053406 | TATGACATGATTACGAATTTAATACGACTCACTATAGGGAATTTGGCCCTCGAGGCCAAGAATTCGGCACGAGGGTTTG |
| | | CCGTGAGAACGCAGGTGTTGTGAAAGCCACCGCTAATTCAAAGCAAAAATGGGAAAGGAAAAGACTCACATCAACATT |
| | | GCCTATTGGCTGCACCCCAGGACCAGTGCCCAGATCCACTTGCTTGGAAACATCGTGATCTGGACTTCAGCCAGCCTC |
| | | GCCACAGTGGCATACACCCTACTGTTCTTCTGGTACCTGCTCCGCCGTCGAAGGAACATCTGTGACCTCCCTGAGGAT |
| | | GCCTGGTCCCACTGGGTGCTGGCTGGAGCTCCAGGAATGAATTCCAATTTCATCTCAAGAGCCCTGTTTGAAGTGATTT |
| | | CTCCTCACACAGTGAAGAATGTGCCCAGCCACAGCATCACCCATGAGGCCCAACTCTGACCAGTGTTTGAGCTGCCAG |
| | | TGTAGGACTCACCTACACTACACCTAAGGCAGGAGGAGCAGCCAGTGAAGGAGTGAAGTCCAGGCCCGGCCCAGCTG |
| | | TGCGCCCACCAATGGGGTCTTAGCTCTCTCCCGANGCCCACAGTACTGCCACTCATTTGTGTGCAGGTACAGTGGCCC |
| | | TCTGTAAAGCCTGCTTTGAAANCTGCCTTCACTCACACTGACTCCTCACCAATGCGACTCTANAAATC |
| Protein tyrosine phosphatase, receptor | NM_019140 | GGGGATCGGTGNGGNCTTTTTNGTTATTACGCCAGCTGGCGAAAGGGGNATGTGCTGCAAGGCGATTAAGTTGGGTA |
| type, D | | ACGCCAGGGTTTTCCCAGTCACGACGTTGTAAAACGACGGCCAGTGCCAAGCTAAAATTAACCCTCACTAAAGGGAAT |
| | | AAGCTTGCGGCCGCCACACNNNTTTTTTTTTTTTNNNTNNNNNNNNNNNNNGGTCCAAGCCAAAGGTAACTTTATTGAT |
| | | TCATGAATCTGGAAAGTTCAAGTACAGGTTTTCCAGAAAAGAACTGATTCTGAGCACAGTGTAGTCAGTGCTCCAT |
| | | CACTCTGGGTTGCCCAAAAAAAGACTTGCACAATGATTCCAGTTGACATGCAACATGGACAGAAACATTTCACAAAGCC |
| | | ACACCCATATATGAAGAGCTAGAGGTAATCAGCTGCTGAGAAAAGGACAATCCGTTTTCTTCAGGGATAGACCTCCTGA |
| | | TAGATTATCCAGTCTCAAGTGTTCAACAAAATACAAGAAACACTAAATGTAATCAGTAAGATGGATTACTGATAGATGAT |
| | | AGATAGATAGATAGATAGATAGATAGATAGATAGATAGATAGATAGATAGATGGATGGATGGATGGATGGATCCTCGTG |
| | | CCGAATTCTTGGCCTCGAGGGCCAAATTCCCTATAGTGAGTCGTATTAAATTCGTAATCATGTCATAA |
| PTEN/MMAC1 | AF017185 | GCGAATTGGGCCCTCTAGATGCATGCTCGAGCGGCCGCCAGTGTGATGGATATCTGCAGAATTCGCCCTTATCGCGG |
| | | GATCCCTATTGCAATGTTCAGTGGCGGAACTTGCAATCCCCAGTTTGTGGTCTGCCAGCTAAAGGTGAGGATCTACTCC |
| | | TCCAACTCAGGACCCACGCGGCGGGAGGACAAGCTCATGTACTTTGAGTTCCCTCAGCCATTGCCTGTGTGTGGTGAC |
| | | ATCAAAGTAGAGTTCTTCCACAAACAGAACAAGATGCTCAAAAAGGACAAAATGTTTCACTTTTGGGTAAATACGTTCTT |
| | | CATACCAGGAGCAGAGGAAACCTCAGAAAAAGTGGAAAATGGAAGTCTTTGTGATCAGGAAATCGATAGCATTTGTAGT |
| | | ATAGAGCGTGCGGATAATGACAAGGAGTATCTCGTGCTCACCCTGACAAAAAATGATCTTGACAAAGCAAACAAAGACA |
| | | AGGCCAAGCGATACTTCTCTCCAAATTTTAAGGTGAAATTATACTTTACAAAAACAGTAGAGGAACCATCAAATCCAGAG |
| | | GCTACAAAGCTTGGCAAGGGCGAATTCCAGCACACTGGCGGCCGTTACTAGTGGATCCGAGCTCGGTACCAAGCTTG |
| | | ATGCATAGCTTGAGTATTCTATAGTGTCACCTAAATAGCTTGGCGTAATCATGGTCATAGCTGGTTCCTG |
| Pyruvate kinase, muscle | M24361 | TATGACATGATTACGAATTTAATACGACTCACTATAGGGAATTTGGCCCTCGAGGCCAAGAATTCGGCACGAGGCGCAT |
| | | GCAGCACCTGATTGCCCGAGAGGCAGAGGCTGCCATCTACCACTTGCAGTTATTCGAGGAACTCCGCCGCCTGGCGC |
| | | CCATTACCAGCGACCCCACAGAAGCTGCCGCCGTGGGTGCCGTGGAGGCCTCCTTCAAGTGCTGCAGTGGGGCCATT |
| | | ATCGTGCTCACCAAGTCTGGCAGGAGTGCTCACCAAGTGGCCCGGTACCGCCCAAGGGCTCCTATCATTGCTGTGAC |
| | | ACGCAATCCCCAGACAGCCCGCCAGGCCCATCTGTACCGTGGCATCTTCCCTGTGCTGTGTAAGGATGCCGTACTGG |
| | | ATGCCTGGGCTGAGGACGTTGATCTTCGTGTGAACTTGGCCATGAATGTTGGCAAGGGCCGAAGCTTCTTCAAGAAAG |
| | | GAGATGTGGTCATTGTGCTGACTGGATGGCGCCCTGGCTCTGGCTTCACCAACACCATGCGTGTANTGCCTGTACCAT |
| | | GATGATCCTCTGGAGCTTCTCTTCTAACCCCTGTCCCTTCCCCTCCCCTATNCTATTCAATTAAGGCACAACGCTTGTAGT |
| | | GCTCACTCTGGGNCATAATGTGGCGCTGGTGGGCTGGGACACCACGAAAAAATAATGNCTCTGAA |
| Ref-1 | D44495 | GNATNTGCTCCACNANAATANAANGATGNANCNTAANNANAATTTAGCAANNATATANNNNNGNCNTNGANNNNNNANA |
| | | NNNNNTTCCAGTGNNGATGGAATATTNTGCAANAATNTCGCCCNTTAATCGCNGGGATCCCGAAANAACCCAAGTCCG |
| | | AANCCAGAAGACCAAAANNANTAAGGGGNCANCAAAANAAAATTTAGAAGGAGNCCNCAAGGAAAAGGGGCCCTTGTCN |
| | | TGTATGAGGACCCNTCCAAGTTCAAGAAAACGTCAGCCAGTGGCAAAATCTTGCCCACACTCAAAAATATGCTCCTGGA |
| | | ATNGGGATGGGCTTCGAGCCTGGATTAAAAGAAAGGCTTGGATTGGGTAAAGGAAGAAGCACCAGACATNTTGTGCC |
| | | TCCAAGAGACCAAATGCTCAGAGAACAGACTTCCCGGCTGAACTGCAAGAGCGTGCCTGGACTCACCCATCAGTACTG |
| | | GTCAGCCCCATCAGACAAAGAAGGATATAGTGGTGTGGGCCTACTTTCCCGCCAATGCCGCTCAAAGTCTCTTATGGC |
| | | ATTGGTGAGGAAGAACATGATCAAGAAGGCCGGGTGATTGTGGCTGAATTTGAGTCCTTTATCTTGGTAACAGCCTATG |
| | | TTCCGAACGCAGGAAGGGGTCTGGTAAGACTGGAGTACCGACAGCGATGGGATGAAGCCTTCAGAAGTTTCTAAAG |
| | | GACTTGGCTTCCCGGAANCATCTTGTGCTGTNTGNAANGNANTTAAANAAANCATN |
| Retinol dehydrogenase type III | U33501 | GNNNNNNCTATGACATGATTACGAATTTAATACGACTCACTATAGGGAATTTGGCCCTCGAGGCCAAGAATTCGGCACG |
| | | AGGGCCTTCTCAGACTCCCTCAGGAGGGAGCTCACCTACTTTGGGGTGAAGGTGGCTATTATAGAGCCTGGTGGGTT |
| | | CAAGACCAATGTCACTAATATGGAGAGGCTATCAGACAACCTGAAGAAGCTGTGGGACCAGGCCACTGAGGAGGTCA |
| | | AGGAGATCTACGGCGAGAAGTTTCGGGACTCCTATATGAAAGCAATGGAGTCAGTGGTGAACATGTGCTCAGGGGAC |
| | | CTGTCTCTGGTAACCGACTGCATGGAGCACGCCCTGACTTCCTGTCACCCTCGCACCCGGTACTCAGCTGGTTGGGAT |
| | | GCCAAGTTCTTCTACCTCCCCATGAGCTACCTTCCCACCTTTCTTTCGGATGCCGTAATCTACTGGGGCTCTGTAAAGC |
| | | CTGCCCGAGCCCTGTGAATCTGCACATGTGTGCAGACTTGGGGAAGTAAGGCGGGTGGAGGGAGATAACAATGTGGG |
| | | GTCCATTGTTCACCATACTCATTAAAATAATTCTGCTTCCGTACTAAAAAAAAAAAAAAAAAAAAAAAAAAAAGTGTGGCG |
| | | GCCGCAAGCTTATT |
| Ribosomal protein L13A | X68282 | GGGAATTGGCCCTCTAGATGCATGCTCGAGCGGCCGCCAGTGTGATGGATATCTGCAGAATTCGCCCTTCGCGGAATT |
| | | CCATTGTGGCCAAGCAGGTACTGCTGGGCCGAAAGGTGGTGGTTGTACGCTGTGAGGGCATCAACATTTCTGGAAATT |
| | | TCTACAGAAACAAGTTAAAGTATCTGGCCTTTCTCCGAAAGCGGATGAACACCAACCCGTCTCGAGGCCCCTACCACTT |
| | | CCGAGCCCCAAGCCGCATTTTTGGCGCACTGTGCGAGGCATGCTGCCGCACAAGACCAAAAGAGGCCAGGCTGCCC |
| | | TGGAACGCCTCAAGGTGTTGGATGGGATCCCTCCACCCTATGACAAGAAAAAGCGGATGGTGGTCCCTGTTGCCCTCA |
| | | AGGTTGTGCGGCTGAAGCCTACCAGAAAGTTTGCTTACCTGGGGCGTCTGGCTCATGAGGTCGGGTGGAAGTACCAG |
| | | GCAGTGACAGCTACTCTGGAGGAGAAACGGAAGGAAAGGCAAAGATCCATTACCGGAAGAAGAAGCAGCTCTTGAG |
| | | GCTAAGGAAACAGGCAGAAAAGAATGTGGAGAAAAGCTGGCCAAGGGCGAATTCCAGCACACTGGCGGCCGNTACTA |
| | | GTGGATCCGAGCTCGGTACCAACTTGATGCATAGCTTGAGTATT |
| Ribosomal protein S17 | NM_017152 | TTCTATGACATGATTACCAATTTAATACGACTCACTATAGGGAATTTGGCCCTCGAGGCCAAGAATTCGGCACGAGGTT |
| | | TTTTTTTTTTACCGAGACCCGTCAACATGGGCCGCGTTCGCACCAAGACTGTGAAGAAGGCGGCCCGGGTCATCATCG |
| | | AGAAGTACTACACGCGCCTGGGTAATGACTTTCACACCAACAAGCGCGTGTGCGAGGAGATCGCCATCATCCCCAGCA |
| | | AAAACCTTCGGAACAAGATAGCCGGCTATGTCACGCATCTGATGAAGCGGATTCAGAGAGGTCCTGTGAGAGGCATCT |
| | | CCATCAAGTTGCAGGAAGAAGAGAGGGAGAGGAGAGATAATTATGTTCCTGAGGTCTCAGCCCTAGATCAGGAGATCA |
| | | TTGAGGTAGATCCTGACACCAAGGAAATGCTGAAGCTTCTGGACTTTGGCAGTCTCTCCAACCTCCAGGTCACTCAGC |
| | | CTACGGTTGGGATGAATTTCAAAACACCACGTGGAGCTGTTTAATCCATTATGCCATATTTTCAATAAACCTGAAAACAA |
| | | AAAAAAAAAAAAAACAAAAAAAAAAAAAAAAACCTTTGCGGCCGCAAGCTTATTCCCTTTANGNGAGGGTTAATTTTAGC |
| | | TTGGCACTGGGCCGTCGTTTTACAACGNCGNGACTGGGAAAACCCTGGGNGTTACCCAACTTAATCGCCTTGCA |
| Ribosomal protein S8 | NM_031706 | TCTATGACATGATTACGAATTTAATACGACTCACTATAGGGAATTTGGCCCTCGAGGCCAAGAATTCGGCACGAGGGAA |
| | | GCTTTGCGCTTCCTCTTTCGAGCCAGCGCCGAGCGATGGGCATCTCTCGGGACAACTGGCACAAGCGCCGCAAGACC |
| | | GGGGGTAAGAGAAAACCCTACCACAAGAAGCGGAAGTATGAGCTGGGACGGCCGGCCGCCAACACTAAGATTGGCC |
| | | CTCGCCGCATACATAGAGTCCGAGTTCGAGGAGGCAATAAGAAGTATCGTGCTCTGAGATTGGATGTGGGGAACTTTT |
| | | CCTGGGGCTCAGAGTGTTGTACTCGCAAAACAAGGATCATTGATGTTGTCTACAATGCATCCAATAACGAGCTTGTCCG |
| | | CACCAAGACCCTGGTGAAGAACTGCATTGTGCTTATTGACAGCACACCGTACCGACAGTGGTACGAGTCCCACTATGC |
| | | ACTGCCCCTGGGCCGCAAGAAGGGGGCCAAGCTGACTCCTGAGGAGGAAGAGATTTTAAACAAAAAACGATCAAAGA |
| | | AAATTCAGAAGAAATATGATGAAAGGAAAAAGAATGCCAAAATCAGCAGTCTTCTGGAGGAGCAGTTCCAGCANGGCA |
| | | AGCTTTCTCGCCTGTATTGGCTCAAGAACAGGCCAGTGTGGCAGANCAGATGGCTATGTGCTCNAANGCAANGAGCTG |
| | | GAGT |
| Ribosomal protein S9 | X66370 | AGCTATTTAGGTGNCACTATAGAATACTCAAGCTATGCATCAAGTTTGGTACCGAGCTCGGATCCACTAGTAACGGCCG |
| | | CCAGTGTGTTGGAATTCGCCCTTCGCGGAATTCTATGTGACCCCACGGAGACCCCTCGAGAAATCGCGTCTCGACCAG |
| | | GAGCTAAAGTTGATTGGAGAGTATGGGCTCCGGAACAAACGTGAGGTGTGGAGGGTCAAATTTACCCTGGGGAAGATT |
| | | CGTAAGGCTGCCCGGGAGCTGTTGACGCTGGACGAGAGGATCCTCGGCGTCTGTTTGAAGGCAACGCTCTGCTGAG |
| | | ACGACTTGTTCGAATTGGGGTGCTGGATGAGGGCAAGATGGAGCTGGATTACATTCTGGGCCTGAAGATTGAGGATTT |
| | | CTTGGAGAGAAGGCTGCAGACCCAGGTGTTTAAGCTGGGCCTGGCCAAATCTATTCACCATGCGCGTGTGCTCATCCG |
| | | CCAACGTCACATCAGGGTCCGCAAGCAGGTGGTGAACATTCCATCTTTCATTGTTCGCCTGGACTCTCAGAAGCACATT |
| | | GACTTCTCCCTCCGTTCTCCTTATGGTGGCGGCCGCCCAGGACGAGTGAAGAGGAAAGCTTGGCCAAGGGCGAATTC |
| | | TGCAGATATCCATCACACTGGCGGCCGCTCGAGCATGCATCTAGAGGGCCCAATTCACAA |
| Sarcoplasmic reticulum calcium ATPase | X15635 | GGAATAGGCCCNCNNGATGATGCTCGAGCGGCCNCCAGTGTGATGGATATCTGCAGAATTCGCCCTTTGCAGAAATGT |
| | | AAGGGTGTTCGGGTGCGTGCATGTGCGTTGTTAGCAACACATCTTCCAGCCCTCTGCATGACTGAGCTTGGGGAAAGA |
| | | GAAATAGAACAGCCCCCAGCTCACTGTGTGATGTGGAGGAAATGTGTATTACAAGTGGGGTTTTAGCTGTTGAGTCAAA |
| | | ATAATAACAAGTGTACAATTTAGCATAAGGAATCGGAGAGCCTCTCCAGAGAAGTCGGTTTCTTTGCTGCAAGAAGAAT |
| | | GAGGTTCTGAACCCTTATCCAAGAACAGAAGCCATCAGCCAAGTCTCCACATTTCTCTGCAAAATGTTGTAGCCTCTAT |
| | | AACTGNATGATAGTGNAATGCATGCCTTCAGTTGTAAGTGGCCCAGATCGCGCTTCAAN |
| Selenoprotein P | NM_019192 | TTATGACATGATTACGAATTTAATACGACTCACTATAGGGAATTTGGCCCTCGAGGCCAAGAATTCGGCACGAGGGGCA |
| | | AGTGAAGGCTTGCAACTTTCACTTGCCCAGAGGAAGCTCTGACGAAGGGGATGCATAAACCAGCTCCTGTGTAAGTTA |
| | | TCTGAGGAGTCTGGGGCAGCTACCAGTAGCTGCTGCTGCCACTGCCGACACCTCATATTTGAGAAGTCAGGATCTGCA |
| | | ATCACTTGACAGTGTGCCGAAAACGTCCCATCCTTGTGTAGCTGACAGGGGCTTTTCGCGGAGGAGAAAGTCATTGAA |
| | | TCCTGTCAATGTAGATCAGCTCCAGCTGCCTGAGACAGTCAGCATGTAAGCCCCACAGAAGCCAGCCCCAACTGAAGC |
| | | TGAAATAATAAGACCAAGAAGTGAAAATGAAATTTGAACTAAATATTTAAATAAAGCGTACTCTCCCCAACTCCATCTAA |
| | | AGACACAATTTCATTTCTAGAATGTTCCAATCCATTTAATTAATTAGTGAAGTAAGTAGTTGAAATTGGATTTGTGCA |
| | | AACATGGAGAAATCTACCACATTGGCTTCTAAAATTTAAAATTTTTATGCCACAAACCATTTCATCCAAATCAGATTTGTA |
| | | CCGTGGGGCAACTGAAAAGTGATTGCGGNCATTGGGTAATATGGCTTTCTTTTCTTTCC |
| Senescence marker protein-30 | X69021 | GAATTGGGCCCTCTAGATGCATGCTCGAGCGGGCGCCAGTGTGATGGATATCTGCAGAATTCGCCCTTATCGGGGGAT |
| | | CCCCAAGGGTCCTTGTACTCCCTTCTTCCTGATCACAGTGTGAAGAATACTTTGACCAAGTGGATATCTCCAATGGTTT |
| | | GGATTGGTCCCTGGACCATAAAATCTTCTACTACATTGACAGCCTGTCCTACACTGTGGATGCCTTTGACTATGACCTG |
| | | CCAACAGGACAGATTTCCAACCGCAGAACTGTTTACAAGATGGAAAAAGATGAACAAATCCCAGATAGAATGTGCATTG |
| | | ATGTTGAGGGGAAGCTTGGGGTGGCCTGTTACAATGGAGGAAGAGTAATTCGCCTAGATCCTGAGACAGGGAAAAGA |
| | | CTGCAAACTGTGAAGTTGCCTGTTGATAAAACAACTTCATGCTGCTTTGGAGGGAAGGATTACTCTGAAATGTACGTGA |
| | | CATGTGCCAGGGATGGGATGAGCGCCGAAGGTCTTTTGAGGCAGCCTGATGCTGGTAACATTTTCAAGATAACAGGTC |
| | | TTGGGGTCAAAGGAATTGCTCCATACTCGGGCAAAGGGCGAATTCCAGCACACTGGCGGCCGTTACTAGTGGATCCG |
| | | AGCTCGGACCAAACTTGATGCATACTTGAGTATTCTATAGTGNCACCTAAATAGCTTGGCGTAATCN |
| Sodium/bile acid cotransporter | M77479 | AGGGGCAGTGAGGGCAACGCAATTAATGTGAGTTAGCTCACTTCATTAGGCACCCCAGGCTTTACACTTTATGCTTCC |
| | | GGCTCGTATGTTGTGTGGAATTGTGAGCGGATAACAATTTCAGACAGGAAACAGCTATGACCATGATTACGCCAAGCTA |
| | | TTTAGGTGACACTATAGAATACTCAAGCTATGCATCAAGCTTGGTACCGAGCTCGGATCCACTAGTAACGGCCGCCAGT |
| | | GTGCTGGAATTCGCCCTTCGCGGGATCCTCCAAAAGGCCACACTATGTACCCTACATCCTCAAGCTGCAGACGCACCA |
| | | TCAGCATGGAAACAGGATTCCAAAACATTCAACTCTGTTCTACCATCCTCAATGTGACCTTCCCCCCTGMGTCATTGG |
| | | GCCACTTTTCTTCTTTCCTCTCCTCTACATGATTTTCCAGCTTGCAGAAGGACTTCTCATCATCATTATCTTCCGGTGCTA |
| | | TGAGAAAATGAAGCCTCCAAAGGACCAAACAAAAATTACCTACAAAGCTGCTGCAACTGAGGATGCTACTCCAGCAGCT |
| | | CTGGAAAAAGGTACCCACAATGGGAAGCTTGGCCAAGGGCGAATTCTGCAGATATCCATCACACTGGCGGCCGCTCG |
| | | AGCATGCATCTAGAGGGCCAATTC |
| Stathmin | NM_017166 | TCTATGACATGATTACGAATTTAATACGACTCACTATAGGGAATTTGGCCCTCGAGGCCAAGAATTCGGCACGAGGCAG |
| | | GCGGCTCGGACTGAGCAGGGCTTTCCTTGCCAGTGGATTGTGTAGAGTGTACAGCCAGTCTCTTGTCTTCTGTCCAAC |
| | | ATGGCATCTTCTGATATTCAGGTGAAAGAGCTGGAGAAGCGTGCTTCCGGCCAGGCCAGGCTTTTGAGCTGATTCTCAGCCCT |
| | | CGATCAAAAGAATCTGTCCCCGAGTTCCCCCTTTCCCCCCCCAAAGAAGAAGGATCTTTCCCTGGAGGAAATTCAGAA |
| | | GAAATTAGAAGCTGCAGAAGAAAGACGCAAGTCTCATGAAGCAGAAGTCTTGAAGCAGCTCGCTGAGAAGCGGGAGC |
| | | ATGAAAAAGAAGTGCTCCAGAAAGCCATTGAGGAGAACAACAACTTCAGCAAAATGGNAGAGGAGAAACTGACCCACA |
| | | AAATGGAGGCTAACAAAGAGAACCGGGAGGCGCAAATGGCTGCCAAGCTGGAGCGTTTGCGAGAGAAGGACAAGCA |
| | | CGTTGAAGAGGTGCGGAAGAACAAAGAATCCAAAGACCCCGCGGACGAGACCGAGGCTGACTAAGTTGTTCCGAGAA |
| | | CTGACTTTCTCCCGACCCCTTCCTAAATATTCANAGACTGTACTGGNGCAG |
| Stearyl-CoA desaturase, liver | J02585 | TATGACATGATTACGAATTTAATACGACTCACTATAGGGAAATTTGGCCCTCGAGGCCAAGAATTCGGCACGAGGCCAGA |
| | | ACATCAGCTCAGGAGGACACGCTGAAACTCTCACTCTGGGCGAGTCCTGATAAAACAGGCTCCTAATACTGACCTACC |
| | | TCAAGGGCAGTTCTGAGGTGATTAGAGCCTTTTCTTTTTTTAAGCAAGTGTTTGGAGATGCATGGCAGAGGTTTTCCTG |
| | | CAGACTTGTAAAGAGGTAGATGTGTTCTAAAGAGCATCTTTAGTCTACATTGCTCTTCCCAGAGATCTACAACTATGACC |
| | | CAGCCAAAGTGCAAGCCTACCTACTGACTGCCATAAAAAGTATTCAGGAAACCTTTTCGTGGCCTGAATGAGATTTTCTT |
| | | TTTTTTTTTTTATGTGGGGCGGTTATTTGTGACCCAAGTGTAATTTGGATGATTTTTAATTAATATCAACTCTTGAAGCCTA |
| | | ATTGTACTGATTTGAGATTGCATTTGTTCCTAATAAAAGTGGATCTAGCTGTGCTGCCAAANNAAAAANNCNANCAAAAA |
| | | AATTGGAAGCGGCCGCAAGCTTATTCCCTTAGNGAGGGTTAATTTTAGCTTGGCACTGGCCGTCGTTTTACCAACGTC |
| | | GNGACTGGGAAAAACCCTGGGGGTTACCCAACTTAATCNCCCTTGCCACGGGNNGGGGGGGNNNGGN |
| Superoxide dismutase Mn | Y00497 | TTTNTGAATTGGGCCCTCTAGATGCATGCTCGAGCGGGCGCCAGTGTGATGGATATCTGCAGAATTCGCCCTTATCGC |
| | | GGGATCCACAAGCACAGCCTCCCTGACCTGCGTTACGACTATGGCGCGCTGGAGCCGCACATTAACGCGCAGATCAT |
| | | GCAGCTGCACCACAGCAAGCACCACGCGACCTACGTGAACAATCTGAACGTCACCGAGGAGAAGTACCACGAGGCGC |
| | | TGGCCAAGGGAGATGTTACAACTCAGGTGCTCTTCAGCCTGCACTGAAGTTCAATGGCGGGGGCCATATCAATCACA |
| | | GCATTTTCTGGACAAACCTGAGCCCTAAGGGTGGTGGAGAACCCAAAGGAGAGTTGCTGGAGGCTATCAAGCGTGAC |
| | | TTTGGGTCTTTTGAGAAGTTTAAGGAGAAACTGACAGCTGTGTCTGTGGGAGTCCAAGGTTCAGGCTGGGGCTGGCTT |
| | | GGCTTCAATAAGGAGCAAGGTCGCTTACAGATTGCCGCCTGCTCTAATCAGGACCCACTGCAAGGAACCACAGGCCTT |
| | | ATTCCACTGCTGGGGATTGATGTGTGGGAGCACGCTTACTATCTTCAGTATAAAAACGTCAGACCTGACTATCTGAAAA |
| | | GCCATTTGGAATGTAATCAACTGGGAGAATGTTAGCCAAAGAAN |
| T-cell cyclophilin | M19533 | GANANNCNACAANNANGGNNANNGAGGNNNGNNNNNNCNNTCCCAGTGTGATGGATATCTGCAGAATTCGGCCCTTA |
| | | TCGCGGGATCCCCTGTTCGAGCTGTTTGCAGNCAAAGTTCCAAAGACAGCAGAAAACTTTCGTGCTTTGAGCACTGGG |
| | | GAGAAGGATTTGGCATATAAGGGTTCCTCCTTTCACAGAATTATTCCAGGATTCATGTGCCAGGGTGGTGACTTCAC |
| | | ACGCCATAATGGCACTGGTGGCAAGTCCATCTACGGAGAGAAATTTGAGGATGAGAACTTCATCCTGAAGCATACAGG |
| | | TCCTGGCATCTTGTCCATGGCAAATGCTGGACCAAACACAAATGGTTCCCAGTTTTTTATCTGCACTGGCAAGACTGAG |
| | | TGGCTGGATGGCAAGCATGTGGTCTTTGGGAAGGTGAAAGAAGGCATGAGCATTGTGGAAGCCATGGAGCGTTTTGG |
| | | GTCCAGGAATGGCAAGACCAGCAAGAAGATCACCATCTCCGACTGTGGACAACTCTAATTTCTTTGACTTGCGGGCATT |
| | | TTACCCATCAAACCATTCCTTCTGTAGCTCAGGAGAGCACCCCCACCANNNNNGNNATNA |
| Thymidylate synthase | L12138 | TTGGCGAATTGGGCCCTCTAGATGCATGCTCGAGCGGCCGCCAGTGTGATGGATATCTGCAGAATTCGCCCTTCGCG |
| | | GGATCCGACAGGAAGGAGACCTGGGCCCAGTTTATGGATTCCAGTGGAGACATTTTGGAGCAGACTACAAAGATATGG |
| | | ATTCAGATTACTCGGGTCAAGGAGTAGACCAGCTGCAAAAAAGTGATTGACACCATCAAAACCAACCCCGATGACAGAA |
| | | GAATCATCATGTGTGCCTGGAACCCAAAAGATCTTCCCCTGATGGCACTGCCTCCTTGCCATGCCCTCTGTCAATTTTA |
| | | TGTGGTGAATGGGGAGCTGTCTTGCCAGCTTTACCAGCGGTCAGGAGATATGGGTCTGGGTGTGCCCTTCAACATTGC |
| | | CAGCTATGCTCTGCTGACCTACATGATTGCACATATCACGGGCCTGCAGCCGGGTGATTTTGTCCATACTTTGGGAGAT |
| | | GCACACATTTATCTGAATCATATTGAGCCACTGAAATTCAGCTACGGCGAGAACCAAGACCTTTCCCAAAGCTCAGAA |
| | | TCCTCCGAAAAGTTGAGACAATCGAAAGCTTGGCCAAGGGCGAATTCCAGCACACTGGCGGCCGTTACTAGTGGATCC |
| | | GACTCGGTACCAAGCTTGATGCATAGCTTGAGTATTCTATAGTGNCACCTAAATAGCTTGN |
| Thymosin beta-10 | M17698 | NTTGTGGGAATTGTGAGCGGATACCATTTTCACACAGGAAACAGTTATGCCATGATTACGCCAAGCTATTTAGGTGACA |
| | | CTATAGAATACTCAAGCTATGCATCAAGCTTGGTAGCGAGCTCGGATCCACTAGTAACGGCCGCCAGTGTGCTGGAAT |
| | | TCGCCCTTCGCGGGATGCGGAGTACCTGGAGCGCGAGCTCGGAACGAGAATCCACGAGTTGTAAGAAAATGGCAGAC |
| | | AAGGCGGACATGGGGGAAATCGCCAGCTTCGATAAGGCCAAGCTGAAGAAAACCGGGACGCAGGAGAAGAACACCCT |
| | | GCCGAGCAAAGAGACCATTGAACAGGAAAAGAGGAGTGAAATCTCCTAAAAGCCTAGGAAGATTTCCCCACCCCACCC |
| | | CTTCATCTCCGAGAACCCCCTCGTGATGTGGAGGAAGAGCCACCTGCAAGATGGACGCGAGCCACAAGCTGCACTGT |
| | | GAACCCGGGCACTCCGCGCCGATGCCACCGGCCCGTGGGTCTCTGAAGGGGACGCGCCCACTAATCGGACTGCCAA |
| | | ATTTCACCGGTTTGCCCAGGGATATTATAAGCTTGGCCAAGGGGGAATTCTGCAGATATCCATCACACTGGCGGCCGC |
| | | TCGAGCATGCATCTAGAGGGCCCAATTCACNC |
| Transthyretin | X14876 | TAGGTGNCACTATAGAATACTCAAGCTATGCATCAAGCTTGGTACCGAGCTCGGATCCACTAGTAACGGCCGCCAGTG |
| | | TGCTGGAATTCGCCCTTATGCGGGATCCTCCAAGTGTCCTCTGATGGTCAAAGTCCTGGATGCTGTCCGAGGCAGCCC |
| | | TGCTGTCGATGTGGCCGTGAAAGTGTTCAAAAAGACTGCAGACGGAAGCTGGGAGCCGTTTGCCTCTGGGAAGACCG |
| | | CCGAGTCTGGAGAGCTGCACGGGCTCACCACAGATGAGAAGTTCACGGAAGGGGTGTACAGGGTAGAACTGGACAC |
| | | CAAATCGTACTGGAAGGCTCTTGGGATTTCCCCATTCCATGAATACGCAGAGGTGGTTTTCACAGCCAATGACTCTGGT |
| | | CATCGCCACTACACCATCGCAGCCCTGCTCAGCCCGTACTCCTACAGCACCACTGCTGTCGTCAGTAACCCCCAGAAC |
| | | TGAGGGACCCAGCCCAGGAGGACCAGGATCTTGCCAAAGCAGTAGCTTCCCATTTGTACTGAAACAGTGTTCTTGCTC |
| | | TATAAACCGTGTTAGGAACTCGGGAAGATGCCGTGAAACGTTCTTAAGCTCAAGGGCGAATTCTGCAGATATCCATCAC |
| | | ACTGGCGGCCGCTCGAGCATGCATCTAGAGGGCCCAATTCGC |
| Tryptophan hydroxylase | X53501 | GANNGGGCCCTCTAGATGCATGCTCGAGCGGCCGCCAGTGTGATGGATATCTGCAGAATTCGCCCTTATCGCGGGAT |
| | | CCAGGATTGGAGCACGTGTGGTGTCAGCAGTGTAGCCTTGGCATAAGCAGTTGTATAAAGTTTTCATTACTGTAACAAA |
| | | GTGTCAGAGACAATCAGCTTATAAAGAGGAAAAGTTTGTTTTGGGTGTTATGGTCCATTAGACGTCGTTCCTGTGGTTT |
| | | AAGCCAGTGGTGAAACAAGGGATGATGAGAGAATGTGGCAGAGCAAACTTCATAAAGGATGGAAAGAGAAAAAGTGAA |
| | | GGGTTTAGAGAAAGAAGAAGAGAGAGATGGAATGAGAGAGGGAGAACACACACATGGGTACGCGCACACACACACAC |
| | | ACACACACACACACACACACACACACACACACACACACACGGGGTGAGAGAGAGTCAGAGACAGACAGAAAGATCCT |
| | | ACAGTACAGTTTAAGAGTTCATTACCAGGGTAAGAGAGAGGGCTCAGCAGTTAAGAGTACTCGCTGCTCTTCCAGAGG |
| | | ACCCTGCTTTGATTCCCAGCACCCACACTGTAAGCTTGGCCAAAAGGGCGAATTTCCAGCACACTGGCGGCCGTTACTA |
| | | GTGGATNCGAGCTCGGNCCCAAGCTTGATGCATAGCTTGAGTATTCTATAGTGTCACCTTAAATAGCTTTGGCGTAATC |
| | | ATTGGNCA |
| Ubiquitin conjugating enzyme (RAD 6 | M62388 | TTGNGAATTGGGCCCTCTAGATGCATGCTCGAGCGGCCGCCAGTGTGATGGATATCTGCAGAATTCGCCCTTCGCGG |
| homologue) | | GATCCGTAGGAGGCTCATGCGGGATTTCAAGCGATTGCAAGAGGACCCACCTGTGGGGGTCAGTGGTGCACCATCTG |
| | | AAAACAACATCATGCAGTGGAACGCAGTTATATTTGGACCAGAAGGGACACCCTTTGAAGATGGTACTTTTAAACTAGT |
| | | AATAGAATTTTCTGAAGAATATCCAAATAAACCACCAACCGTTAGGTTTTTATCCAAAATGTTTCATCCAAATGTGTATGC |
| | | TGACGGCAGCATATGCTTAGACATCCTGCAGAACCGATGGAGCCCCACGTACGACGTCTGCTCCATCTTAACTTCAATT |
| | | CAGTCTCTGTTGGATGAGCCGAATCCAAACAGTCCGGCCAATAGCCAAGCAGCACAGCTTTATCAGGAAAACAAACGG |
| | | GAGTATGAGAAGAGGGTTTCGGCCATTGTTGAGCAGAGCTGGAATGACTCATAATAGACACCTGGTCTGTGCACCTTT |
| | | CCATCGTCGTTGTGAAGCTTGGCCAAGGGCGAATTCCAGCACACTGGCGGCCGTTACTAGTGGATCCGAGCTCGGTA |
| | | CCAAGGTTGATGCATAGCTTGAGTATTCTATAGTGCACCTAAATAGCTTGGCGTAA |
| Uncoupling protein 2 | AB006613 | NGGGGAATTGGGCCCTCTAGATGCATGCTCGAGCGGCCGCCAGTGTGATGGATATCTGCAGAATTCGCCCTTCGCGG |
| | | AATTCTGGCAGGAGCACCACAGGTGCCCTGGCTGTGGCTGTGGCCCAACCTACAGATGTGGTAAAGGTCCGCTTCCA |
| | | GGCCCAGGCCCGGGCTGGCGGTGGTCGGAGATACCAGAGCACTGTCGAAGCCTAGAAGAGCATTGCACGAGAGGAA |
| | | GGGATCCGGGGCCTCTGGAAAGGGACCTCTCCCAATGTTGCCCGAAATGCCATTGTCAACTGTACTGAGCTGGTGAC |
| | | CTATGACCTCATCAAAGATACTCTCCTGAAAGCCAACCTCATGACAGACGACCTCCCTTGCCACTTCACTTCTGCCTTC |
| | | GGGGCGGGCTTCTGCACCACCGTCATTGCCTCCCCCATTGATGTGGTCAAGACGAGATATATGAACTCTGCCTTGGGC |
| | | CAGTACCACAGCGCCGGCCACTGTGCCCTGACCATGCTCCGGAAGGAGGGGCCCCGAGCCTTCTACAAGGGGTTCAT |
| | | GCCTTCCTTCCTCCGCTTGGGATCCTGGAACGTAAAGCTTGGCCAAGGGCGAATTCCAGCACACTGGCGGCCGTTACT |
| | | AGTGGATCCGAGCTCGGACCAACTTGATGCATAACTTGAGTATTCTATAGTGGCA |
| Urinary protein 2 precursor | AF198441 | CTATGACATGATTACGAATTTAATACGACTCACTATAGGGAATTTGGCCCTCGAGGCCAAGAATTCGGCACGAGGGTTT |
| | | GAAGATCAATCTCACTGCAATGGGAAAGCCTATCTTGCTGCTCCCACTGGGCCTGTCTTTGCTCATGAGCTCCCTGCTA |
| | | GCTTTGCAGTGCTTCAGATGTGAAAGCCTCGATTCTACAGGACTTTGTCGTGTTGGAAGACGTATATGTCAGACTTATC |
| | | CTGATGAGATTTGTGCCTGGGTTGTAGTAACCACTCGAGATGGAAAATTTGTATATGGAAACCAGAGCTGTGCTGAGTG |
| | | CATCGGTACGACTGTTGAGCATGGAAGTCTTATAATATCAACAAACTGTTGCTCAGCCACCCCTTTCTGCAATATGGTA |
| | | CATCCCTGATACTTAATTGTATGGAAGGATTTTAGTGCTCTTTATTGAGTCTTGACAGTGGAGCATGCGAGAAAGAATAA |
| | | TCTACTGACCATGTCTGGACAACTCACTATGATTTCTACATCGTGTTTTCTGACTACATGTATATCATATGTTCTTGGGTG |
| | | ATCTGTAATAAATTGAAAGCCTTACATATTTTAATTTGAACTAAAAAAAAAAAAAAAAAAAAAAAAAAAGTGTGGCG |
| Voltage-dependent anion channel 2 (Vdac2) | NM_031354 | CTATGACATGATTACGAATTTAATACGACTCACTATAGGGAATTTGGCCCTCGAGGCCAAGAATTCGGCACGAGGGTCT |
| | | CCTTCACTTCGCCCTCCAGCCGCGGTGGCTGCAGCGCAACTTCCAGATAGCGGAGTGGCCTCAGCTGCGAGCCGAGC |
| | | GGTGGCGGCAGCGCCCCTCAGGACACCCGCAGATCACCTTTTCCCCGCGACTTCGCCATGGCTGAATGTTGTGTACC |
| | | GGTATGCCAACGGCCAATTTGTATCCCTCCACCCTATGCTGACCTTGGCAAAGCTGCCAGAGATATTTTCAACAAAGGA |
| | | TTTGGTTTTGGGTTGGTAAAGCTGGATGTGAAAACGAAGTCATGCAGTGGTGTGGAATTTTCAACATCTGGCTCATCTA |
| | | ATACAGACACTGGTAAGTCAGTGGGACCTTGGAGACCAAGTACAAATGGTGTGAGTATGGTCTGACTTTCACAGAGA |
| | | AATGGAACACTGACAACACTCTGGGGACGGAGATTGCAATTGAAGAAGACCAGATTTGTCAAGGTTTGAAACTGACCTTTGA |
| | | CACCACGTTTTCACCAAACACAGGAAAGAAAAGTGGTAAAATCAAGTCTGCTTACAAGANGGAATGTATAAACCTTGGC |
| | | TGTGATGTTGATTTTGATTTTGCTGGGACCTGCCATCCATGGGTCACCCGTNTTTGGNTACGGGGGGGGGGGGGGNG |
| Zinc finger protein | AF001417 | AGGTGTGNAAATGACCGAGGAACTCAGATTTCTTGAATTGACTAAGACTTCACCAATGGGGTCAGAGGTAAACTTTGGT |
| | | CGTGGGCGAAAGTTCNTCCGAACTGTCTGAAGATTCGCTGCTGACATCAGAGTTCAGGCTATTGGTCTCTAAGTTATAA |
| | | TTAAAGCCGGAGCTGATCAGAGAGTCCTCTGGGGGACTAGAAGAAATCTTCAGTTCTGATTCCTCCTTCCTCTCCCGTG |
| | | CTAGAATGATTTTGGTCCACAGGTCTTCCTGGTTGTCAAATTTTATCTCAGAGGCTGACACGTAGCAGGGCTCAGTCTG |
| | | AAGATAGCGTTCCAACTCCAGGCAGGTCTGTTGCCAATATTCCTCCAGGGACGGCAGAGCCGAGAAGTAGCCCGTTTC |
| | | GTGCACAATCTGTAGTTCCTGGAAGATGCTACACATTGGGAGCACATCCATGTCGGGTTGGAAAAGACAGTCCCCGCT |
| | | GTCGGGAAAACAGGGAGGTGAACGATCAGGAGTCGGAGCAGAAACTGTTCGCGGGAGCGCAGGTGAAAGTTTCATGC |
| | | AAACTGGATGGCGCTGCAATCGGACGCCGGGTCCGGACCCTCCCGCAGCCCGCAGCGGGCCGAGCCCACGCAATAT |
| | | TTGCCTCGTGCCGAATTCTTGGCCTCGAGGGCCAAATTCCCTATAGTGAGTCGTATTAAATTCGTAATCATGTCATANN |
| | | NNG |

TABLE 34
Expression Data for 6 Hour Timepoint (1)

| | ANIT | ANIT | ANIT | ANIT | ANIT | ANIT | 5-FU |
|---|---|---|---|---|---|---|---|
| Compound-Dose (2) | 15 | 15 | 15 | 60 | 60 | 60 | 13 |
| Animal Number (3) | 1641 | 1642 | 1643 | 1651 | 1652 | 1653 | 1921 |
| Liver Toxicity Necrosis Classification (4) | no | no | no | yes | yes | yes | no |
| Gene Name (5) | | | | | | | |
| Gadd153 | 0.7185743 | 0.9336073 | 0.8929988 | 0.8827605 | 0.7812951 | 0.9294331 | 0.985154 |
| Interferon related developmental regulator | 0.6898839 | 0.7755275 | 0.7540799 | 0.7127763 | 0.8136663 | 0.7227162 | 0.8944311 |
| IFRD1 (PC4) | | | | | | | |
| ID-1 | 1.2276917 | 1.2966 | 1.2033458 | 1.464322 | 1.2952479 | 1.1469147 | 1.107182 |
| Insulin-like growth factor binding protein 1 | 1.2149296 | 1.2517433 | 0.849152 | 0.8105241 | 0.9387315 | 0.7861077 | 1.624905 |
| NIPK | 1.1653209 | 1.3313817 | 1.0859097 | 1.1544943 | 1.2684283 | 0.9513855 | 1.1618098 |
| Phase-1 RCT-50 | 1.0134555 | 1.0255734 | 0.9750375 | 1.2598153 | 1.1561269 | 1.1628666 | 1.1502093 |
| Sarcoplasmic reticulum calcium ATPase | 0.7396581 | 0.7300929 | 0.7690976 | 0.8404832 | 0.7737302 | 0.811909 | 1.0051636 |
| Phase-1 RCT-207 | 1.289143 | 1.1662071 | 1.1077025 | 1.2380215 | 1.2850202 | 1.2828368 | 1.0282483 |
| Acetyl-CoA carboxylase | 1.2387872 | 1.1087221 | 1.0561755 | 1.0629063 | 0.968881 | 0.993474 | 1.0338572 |
| Cathepsin L, sequence 2 | 0.9216462 | 0.754725 | 0.8318392 | 0.7312754 | 0.8803408 | 0.7271667 | 1.1044714 |
| Glucokinase | 0.9829909 | 0.9807149 | 0.8488332 | 0.8831044 | 0.3740629 | 0.6822068 | 0.8567957 |
| Gadd45 | 0.8634767 | 0.7835796 | 1.2540028 | 0.8263367 | 0.8540667 | 0.5859734 | 1.2413412 |
| Phase-1 RCT-18 | 0.9880852 | 1.0350194 | 0.9587807 | 1.0483129 | 0.9864402 | 0.945038 | 0.9594232 |
| 3-beta-hydroxysteroid dehydrogenase | 1.0688981 | 1.0014876 | 0.9632384 | 0.8352436 | 0.7861581 | 0.8678508 | 1.0936232 |
| (HSD3B1) | | | | | | | |
| Phase-1 RCT-221 | 1.0449207 | 1.0287501 | 0.9411498 | 0.9509047 | 0.9419692 | 0.8646358 | 0.9743303 |
| Heme oxygenase | 1.0908803 | 0.8578102 | 0.8084754 | 0.794832 | 0.8409988 | 0.7560927 | 1.2295612 |
| Cyclin dependent kinase 4 | 0.9099755 | 0.923375 | 0.9630763 | 0.9599556 | 0.8867117 | 0.8782047 | 0.9523531 |
| Phase-1 RCT-75 | 1.3928976 | 0.9574019 | 1.4720993 | 1.5138696 | 1.3571436 | 1.1690391 | 0.8948373 |
| Phase-1 RCT-65 | 1.3233488 | 1.2809784 | 1.3219572 | 1.1647984 | 1.3021673 | 1.1629522 | 1.3819382 |
| Cytochrome P450 1A1 | 3.404732 | 4.175663 | 3.1513543 | 1.3179038 | 9.773597 | 3.9355524 | 0.9577171 |
| Peroxisomal multifunctional enzyme type II | 0.8356373 | 0.9731294 | 1.0771408 | 0.6359808 | 0.9644716 | 0.8670697 | 1.1467946 |
| DNA topoisomerase I | 0.8054658 | 0.9291925 | 0.9849191 | 0.8489508 | 0.8531781 | 0.7827807 | 1.0761061 |
| Focal adhesion kinase (pp125FAK) | 1.0515311 | 0.9712278 | 1.0951585 | 1.0977428 | 1.0256263 | 0.8672704 | 0.9755156 |
| NGF-inducible anti-proliferative putative | 0.8653699 | 0.9457198 | 0.9580259 | 0.9351403 | 0.9743493 | 0.885854 | 1.3307024 |
| secreted protein (PC3) | | | | | | | |
| Phase-1 RCT-82 | 1.013551 | 1.0175092 | 0.9879723 | 1.0159973 | 1.0188105 | 1.0378032 | 0.9737003 |
| Senescence marker protein-30 | 0.5650517 | 0.9331675 | 1.2714597 | 0.6972141 | 0.7086856 | 0.5903618 | 0.9090846 |
| Melanoma-associated antigen ME491 | 1.1123772 | 1.1784738 | 1.2164025 | 1.1503028 | 0.9342504 | 1.0693377 | 1.1366633 |
| Activating transcription factor 3 | 1.452152 | 1.4229136 | 1.3566269 | 1.5028864 | 1.3285861 | 1.4855398 | 1.1027302 |
| Tryptophan hydroxylase | 0.9645554 | 0.9297437 | 0.9538499 | 0.9363961 | 0.8669578 | 0.8997998 | 1.0017949 |
| Zinc finger protein | 1.0606091 | 1.0850496 | 0.9745781 | 1.3638867 | 0.9989352 | 1.0269728 | 1.0491397 |
| c-myc | 1.5147784 | 1.0234506 | 1.1666878 | 1.331713 | 1.2711399 | 1.1695328 | 1.0753578 |
| Cholesterol 7-alpha-hydroxylase (P450 | 0.9303029 | 0.9509728 | 0.5418924 | 0.8818946 | 0.6116998 | 1.2272166 | 0.6835923 |
| VII) | | | | | | | |
| Ferritin H-chain | 0.6835101 | 0.6700283 | 0.6423172 | 0.670822 | 0.7071592 | 0.6641246 | 0.8880591 |
| Phase-1 RCT-197 | 0.9466599 | 0.8464824 | 0.832247 | 0.7768468 | 0.8980972 | 0.8835461 | 1.1923697 |
| Phase-1 RCT-179 | 0.9326698 | 0.8378596 | 1.039075 | 0.9614799 | 1.0280399 | 0.8014495 | 0.8718779 |
| Phase-1 RCT-15 | 1.015028 | 0.9775072 | 0.957975 | 1.002485 | 0.9226243 | 1.0759921 | 1.3613387 |
| Argininosuccinate lyase | 1.2056043 | 1.3548754 | 1.1052656 | 1.4339882 | 1.6074582 | 1.6668192 | 1.1163688 |
| Phase-1 RCT-49 | 0.9294566 | 0.9788385 | 0.9558389 | 1.1491555 | 1.02307 | 1.0143225 | 0.9257543 |
| Carbonic anhydrase III | 0.4845098 | 1.079574 | 0.8744931 | 0.7632172 | 0.9679762 | 1.112431 | 1.1657006 |
| Proliferating cell nuclear antigen gene | 0.8825166 | 0.8918006 | 0.9163802 | 0.9420029 | 1.0223718 | 1.0601966 | 0.8980906 |
| Integrin beta1 | 1.009435 | 1.0328991 | 1.0399166 | 0.9906036 | 0.9890845 | 0.8913259 | 1.0610689 |
| Caspase 6 | 0.8144714 | 0.8695206 | 0.8474238 | 0.7780047 | 0.9078218 | 0.9058883 | 1.0391469 |
| Phase-1 RCT-116 | 1.1774017 | 1.3448613 | 1.0215157 | 1.1908216 | 1.1333046 | 1.1744632 | 1.275086 |
| Phase-1 RCT-109 | 1.0254289 | 0.9660531 | 1.0601807 | 1.0625726 | 1.0412246 | 0.9230573 | 0.8201333 |
| Phase-1 RCT-71 | 1.032324 | 1.1339133 | 1.0255227 | 1.0752836 | 1.0623491 | 1.182517 | 0.8791432 |
| Elongation factor-1 alpha | 0.876109 | 0.8442411 | 0.8653391 | 0.8343079 | 0.8516285 | 0.8428534 | 1.0510715 |
| Phase-1 RCT-169 | 0.9582353 | 0.880698 | 0.8885882 | 1.0733279 | 0.9992086 | 1.0009198 | 1.1951162 |
| Pyruvate kinase, muscle | 0.9535065 | 0.853659 | 0.8753552 | 0.8693974 | 0.9328217 | 0.7706469 | 1.0531771 |
| Nucleosome assembly protein | 0.6089873 | 0.8987361 | 0.8813744 | 0.5589454 | 0.7303363 | 0.8971077 | 0.9334246 |
| Phase-1 RCT-127 | 0.9760207 | 0.9784484 | 0.9201744 | 1.0834774 | 1.1456012 | 1.0976746 | 0.9351027 |
| Iron-responsive element-binding protein | 0.95496 | 1.0916806 | 1.1726447 | 0.8244379 | 1.0485252 | 1.033854 | 0.8922884 |
| Phase-1 RCT-72 | 0.9564911 | 0.8800318 | 0.9346035 | 1.0972357 | 1.0255792 | 1.0620791 | 1.1585289 |
| Phase-1 RCT-144 | 0.9853792 | 0.937196 | 1.0796523 | 1.083639 | 1.2561976 | 0.9939714 | 0.8835513 |
| Phase-1 RCT-242 | 0.9511073 | 1.081147 | 0.9987483 | 1.2816093 | 1.0942544 | 1.1550943 | 1.9052056 |
| 17-beta hydroxysteroid dehydrogenase, | 0.2012994 | 0.8053457 | 0.6630149 | 0.4131254 | 0.6416274 | 0.8495546 | 0.7874646 |
| type 2 | | | | | | | |
| Phase-1 RCT-70 | 1.4160482 | 1.1079257 | 1.0468442 | 1.0817455 | 1.1199657 | 1.0359564 | 1.022524 |
| Ribosomal protein L13A | 1.0169355 | 0.8784845 | 1.0461044 | 1.0611742 | 0.9836401 | 0.8373581 | 0.9043261 |
| Cytochrome P450 2E1 | 1.236674 | 1.3538622 | 1.2765596 | 1.1653274 | 1.0093058 | 1.704629 | 0.7364896 |
| AT-3 | 0.8897899 | 0.885324 | 0.9628465 | 0.8641002 | 0.9821184 | 0.8789114 | 1.0034171 |
| Phase-1 RCT-270 | 1.1011877 | 1.0679487 | 1.1068181 | 0.9381476 | 0.9116659 | 1.0485853 | 0.7675028 |
| Phase-1 RCT-123 | 0.9355506 | 1.0066652 | 0.9724118 | 1.0092225 | 1.0079914 | 1.0441352 | 1.0704002 |
| Alpha-2-macroglobulin, sequence 2 | 0.984683 | 0.7993588 | 0.8231666 | 1.0809472 | 0.8669137 | 1.0587713 | 0.8900628 |
| Matrix metalloproteinase-1 | 0.7543654 | 0.7491251 | 0.7469956 | 0.7463155 | 0.7688904 | 0.7061313 | 0.853909 |
| Beta-tubulin, class I | 1.0952213 | 1.2097276 | 1.7881792 | 1.1133955 | 1.4442486 | 1.0395551 | 0.9839663 |
| Phase-1 RCT-161 | 0.9629833 | 1.008041 | 0.9222848 | 1.0530261 | 1.0164492 | 1.015252 | 1.1400217 |
| 14-3-3 zeta | 1.2609897 | 1.2516987 | 1.293437 | 1.2637444 | 1.3685564 | 1.1688179 | 1.079549 |
| Stem cell factor | 0.7785376 | 0.8548129 | 1.1513512 | 0.8351924 | 0.8007931 | 0.6740239 | 1.0943563 |
| Macrophage inflammatory protein-2 alpha | 1.050731 | 0.9662606 | 0.7755001 | 1.1097608 | 1.2300837 | 0.966158 | 1.0411074 |
| Thymidylate synthase | 0.994112 | 0.9508652 | 1.0184784 | 1.0404687 | 0.953401 | 0.9762096 | 0.9818226 |
| c-jun | 1.1578233 | 1.2684429 | 1.376932 | 1.9909544 | 1.7620658 | 1.5192893 | 2.0682418 |
| Superoxide dismutase Mn | 1.191638 | 1.1185166 | 1.158542 | 1.051313 | 1.0466814 | 1.0973539 | 1.0441002 |
| Phase-1 RCT-73 | 0.8896211 | 1.0080326 | 1.0021784 | 0.8007281 | 0.9510467 | 0.8591147 | 0.9783914 |
| Macrophage inflammatory protein-1 alpha | 1.237433 | 0.9849516 | 0.9835461 | 1.4384342 | 1.2878778 | 1.2832083 | 1.0215358 |
| Phase-1 RCT-214 | 1.0963717 | 1.0286255 | 1.1609334 | 0.953472 | 1.0031888 | 0.9564763 | 1.1001635 |
| NADH-cytochrome b5 reductase | 1.1950442 | 1.2027489 | 1.180445 | 1.1633062 | 1.0699576 | 1.2800499 | 0.9218112 |
| Interleukin-1 beta | 0.8933691 | 0.8187771 | 0.8707685 | 0.9796415 | 0.9708717 | 0.9417814 | 0.9505159 |
| NADP-dependent isocitrate | 1.0143814 | 1.0535113 | 1.3957092 | 0.9624125 | 1.0712386 | 0.9659266 | 0.8232184 |
| dehydrogenase, cytosolic | | | | | | | |
| Phase-1 RCT-289 | 1.1787131 | 1.118199 | 1.1143155 | 1.0494264 | 1.116709 | 1.169125 | 1.0520252 |
| Glutamine synthetase | 1.1762499 | 1.4699069 | 1.2290528 | 0.8384649 | 0.8188794 | 1.0502946 | 1.275435 |
| Phase-1 RCT-182 | 1.1028969 | 0.9852377 | 0.9824249 | 0.8813642 | 0.8578239 | 1.2171141 | 0.9131823 |
| Ubiquitin conjugating enzyme (RAD 6 | 1.1594281 | 0.9303154 | 1.1112452 | 1.0594631 | 1.1553923 | 0.977963 | 1.0631553 |
| homologue) | | | | | | | |
| Extracellular-signal-regulated kinase 1 | 0.5491036 | 1.0574118 | 0.8905959 | 0.8066067 | 1.0395427 | 1.1199929 | 1.1582721 |
| Emerin | 1.0191764 | 1.0759492 | 1.0692528 | 1.0734193 | 1.093064 | 0.982335 | 0.9517204 |
| Phase-1 RCT-78 | 1.0567994 | 1.0444764 | 1.0573443 | 0.9519615 | 1.0125328 | 1.0740681 | 1.1585928 |
| Phase-1 RCT-212 | 0.8336432 | 0.8106836 | 0.8402128 | 1.0186192 | 0.9425325 | 0.8530802 | 0.9507755 |
| Phase-1 RCT-168 | 1.1201595 | 1.111117 | 1.1195064 | 1.0638678 | 1.0535724 | 0.9803076 | 1.0363439 |
| Lysyl hydroxylase | 1.030539 | 1.1117865 | 1.1065004 | 0.8620387 | 0.8845925 | 0.9162976 | 0.9459705 |
| Phase-1 RCT-55 | 1.1767752 | 0.8761249 | 1.0162835 | 1.268858 | 1.2538329 | 0.9329485 | 1.1302994 |
| MHC class I antigen RT1.A1(f) alpha- | 1.9584932 | 1.8475755 | 1.7166697 | 1.6590681 | 1.6042585 | 1.449923 | 1.433592 |
| chain | | | | | | | |
| Phase-1 RCT-40 | 0.8299238 | 0.8246514 | 0.9613408 | 0.7184317 | 0.7115783 | 0.7588074 | 0.8732337 |
| Cyclin G | 0.9861405 | 1.0574917 | 1.0137767 | 0.7868509 | 0.9781407 | 1.047667 | 1.2199423 |
| Peroxisome proliferator activated receptor | 0.9240714 | 0.8528097 | 0.9166532 | 0.9808784 | 0.8266951 | 0.8610089 | 1.1634672 |
| gamma | | | | | | | |
| Phase-1 RCT-280 | 0.9341313 | 1.1864057 | 1.0366496 | 0.9431174 | 0.9929938 | 1.0330125 | 0.7716662 |
| D-dopachrome tautomerase | 0.9663504 | 0.8672113 | 0.9236014 | 0.86123 | 0.9134668 | 0.9512128 | 0.8304919 |
| Phase-1 RCT-287 | 0.885182 | 0.829776 | 1.0057417 | 0.7684604 | 0.9359691 | 0.8276541 | 1.0144409 |
| Phase-1 RCT-119 | 0.9507858 | 0.8301064 | 0.7305186 | 0.9939791 | 0.7619197 | 1.2791598 | 0.8959353 |
| Thioredoxin-1 (Trx1) | 0.7917261 | 0.7348733 | 0.9400805 | 0.8640825 | 0.8413442 | 0.8007128 | 0.8722432 |
| Phase-1 RCT-225 | 0.5667768 | 0.8596091 | 0.7999845 | 1.3692746 | 1.1744996 | 0.7431118 | 1.3262959 |
| Protein kinase C alpha | 1.0431842 | 0.9915947 | 1.0425026 | 1.072689 | 1.104208 | 1.0923749 | 0.9293683 |
| Phase-1 RCT-87 | 0.9755843 | 0.9233789 | 0.9689491 | 0.91749 | 0.9986669 | 0.9628528 | 0.9678066 |
| Phase-1 RCT-59 | 1.2079571 | 1.1136501 | 1.0195632 | 1.0277864 | 1.1726079 | 1.0952358 | 1.0166749 |
| C4b-binding protein | 0.6388986 | 0.7028221 | 0.6216511 | 0.5506674 | 0.6151086 | 0.6679841 | 1.1421845 |
| Phase-1 RCT-204 | 0.9328041 | 0.9965556 | 0.9401906 | 0.9321955 | 0.915742 | 1.0076454 | 1.0095606 |
| Carnitine palmitoyl-CoA transferase | 1.2755116 | 1.6034126 | 1.265878 | 1.2466457 | 2.1334035 | 1.4087862 | 1.3604103 |
| 8-oxoguanine DNA glycosylase | 0.99154 | 0.9938749 | 0.9517627 | 1.203186 | 1.1149639 | 1.0340207 | 1.0808245 |
| H-rev107 | 0.9989693 | 0.9772748 | 1.0796107 | 1.1261469 | 0.9959444 | 0.9453217 | 0.8773518 |
| Preproalbumin | 0.9349386 | 1.1626682 | 1.2016798 | 0.8078082 | 0.8294842 | 1.1399122 | 1.2619064 |
| Phase-1 RCT-177 | 0.7583118 | 0.7633801 | 0.8940682 | 0.5814648 | 0.8069022 | 0.6461482 | 0.9605613 |
| Phase-1 RCT-110 | 0.9485259 | 0.9847435 | 0.9803221 | 0.9402353 | 1.004198 | 1.1107664 | 1.0542169 |
| Phase-1 RCT-227 | 0.8833574 | 1.0183736 | 0.8947167 | 0.6572688 | 0.8903108 | 1.1178145 | 1.2398838 |
| Neuropeptide Y | 0.9952342 | 0.9726904 | 0.9153593 | 1.0660263 | 1.0813193 | 1.0456665 | 1.1304039 |
| Phase-1 RCT-277 | 0.9487815 | 0.7033335 | 1.0747119 | 0.8574905 | 0.9126127 | 0.6481303 | 0.6874526 |
| Phase-1 RCT-229 | 0.9944909 | 0.9842671 | 1.0025935 | 1.0843838 | 1.0881028 | 1.0507909 | 1.1274085 |
| Phase-1 RCT-34 | 1.4664594 | 1.104215 | 1.33316 | 1.2312793 | 1.2677217 | 1.1349192 | 0.7961378 |
| Choline kinase | 1.4081486 | 1.1065426 | 1.2669674 | 0.9868597 | 0.787567 | 0.9823182 | 1.3576117 |
| Phase-1 RCT-20 | 1.079748 | 0.9636784 | 0.9785105 | 0.9745486 | 1.0258286 | 1.060652 | 1.1061754 |
| Phase-1 RCT-248 | 1.0111111 | 1.0348809 | 0.9511109 | 1.0691276 | 1.0556587 | 1.0835474 | 0.9363523 |
| | 5-FU | 5-FU | 5-FU | 5-FU | 5-FU | APAP | APAP |
| Compound-Dose (2) | 13 | 13 | 50 | 50 | 50 | 250 | 250 |
| Animal Number (3) | 1922 | 1923 | 1931 | 1932 | 1933 | 2121 | 2122 |
| Liver Toxicity Necrosis Classification (4) | no | no | no | no | no | no | no |
| Gene Name (5) | | | | | | | |
| Gadd153 | 1.0788583 | 1.0081168 | 1.0519096 | 1.1473383 | 1.2465549 | 1.1727681 | 1.3356233 |
| Interferon related developmental regulator | 1.0828478 | 1.0075552 | 0.6761732 | 1.2359542 | 1.0848296 | 0.9331386 | 1.0134275 |
| 1FRD1 (PC4) | | | | | | | |
| ID-1 | 1.0950371 | 1.01164 | 1.2433068 | 0.9020668 | 0.9879841 | 1.2038897 | 1.2593217 |
| Insulin-like growth factor binding protein 1 | 0.9030647 | 0.8451559 | 1.0900192 | 0.976455 | 0.8919143 | 0.9329634 | 1.0453888 |
| NIPK | 1.1567576 | 0.9219275 | 1.2329302 | 1.0776528 | 1.0960306 | 1.1880271 | 1.1305571 |
| Phase-1 RCT-50 | 1.0498691 | 0.9108583 | 1.0588776 | 1.5152891 | 0.669942 | 1.1071056 | 1.1057713 |
| Sarcoplasmic reticulum calcium ATPase | 0.8858995 | 1.0098606 | 0.7356453 | 0.9549313 | 0.9002085 | 1.137184 | 1.1231465 |
| Phase-1 RCT-207 | 0.9924706 | 1.0921209 | 1.4034077 | 1.958303 | 1.3692966 | 1.1813176 | 1.3113829 |
| Acetyl-CoA carboxylase | 0.9946747 | 1.0601611 | 1.0705246 | 0.9494971 | 0.905874 | 0.8981977 | 0.8990743 |
| Cathepsin L, sequence 2 | 0.9508792 | 0.8724679 | 0.9591028 | 1.516148 | 0.9838328 | 1.0476373 | 1.0222765 |
| Glucokinase | 1.0948236 | 1.5476094 | 0.9257579 | 0.5621789 | 0.9273129 | 0.8981254 | 0.8295217 |
| Gadd45 | 0.8108042 | 0.8448618 | 0.8928306 | 1.1119313 | 1.3372039 | 1.3245273 | 1.4109555 |
| Phase-1 RCT-18 | 1.0059595 | 1.0282006 | 0.957373 | 0.9748662 | 1.0473009 | 1.001077 | 0.9757577 |
| 3-beta-hydroxysteroid dehydrogenase | 1.0432448 | 1.0601586 | 1.0656419 | 0.8712314 | 1.0014613 | 0.7063501 | 0.7752215 |
| HSD3B1) | | | | | | | |
| Phase-1 RCT-221 | 1.1178569 | 1.1038841 | 1.1093571 | 1.0788866 | 1.1596407 | 1.015261 | 1.5466703 |
| Heme oxygenase | 1.26232 | 1.0781766 | 1.0228149 | 2.1180346 | 1.4203475 | 1.6522803 | 1.3570095 |
| Cyclin dependent kinase 4 | 1.0218555 | 0.9387755 | 0.8660192 | 0.9259316 | 0.9589778 | 1.0311471 | 1.0069666 |
| Phase-1 RCT-75 | 1.1647131 | 0.834103 | 1.2567677 | 1.1566842 | 1.0223076 | 1.015826 | 1.0514078 |
| Phase-1 RCT-65 | 1.1425153 | 1.0306206 | 1.3208362 | 1.0988663 | 1.20215 | 1.1361784 | 1.0753907 |
| Cytochrome P450 1A1 | 0.8820159 | 0.8567971 | 1.3876512 | 0.9843562 | 0.9543454 | 1.1943812 | 1.2198145 |
| Peroxisomal multifunctional enzyme type II | 1.0778381 | 1.2019639 | 0.8039252 | 1.169988 | 1.0594281 | 0.8714904 | 0.8896325 |
| DNA topoisomerase I | 1.0567739 | 0.9435328 | 0.7343188 | 1 | 0.9774364 | 0.4803605 | 0.5703286 |
| Focal adhesion kinase (pp125FAK) | 0.9413212 | 0.8969048 | 0.9399666 | 0.9965205 | 0.9535865 | 0.9591684 | 1.0023568 |
| NGF-inducible anti-proliferative putative | 1.073774 | 0.9825605 | 0.956658 | 1.114923 | 0.9335354 | 0.7367932 | 0.8152962 |
| secreted protein (PC3) | | | | | | | |
| Phase-1 RCT-82 | 0.9630044 | 1.0768925 | 0.9286189 | 1.0180067 | 0.9677689 | 1.021052 | 1.068598 |
| Senescence marker protein-30 | 1.0748038 | 0.9792197 | 0.6202778 | 0.4626555 | 0.90999 | 0.5696384 | 0.5276256 |
| Melanoma-associated antigen ME491 | 0.8162779 | 0.7222134 | 1.0494608 | 0.8858706 | 1.2314706 | 0.7085851 | 0.8886442 |
| Activating transcription factor 3 | 0.9054137 | 1.0205317 | 1.2999927 | 1.0627655 | 1.0905476 | 1.2899004 | 1.0307328 |
| Tryptophan hydroxylase | 1.0600281 | 1.0654569 | 0.9482657 | 0.8546203 | 0.9942221 | 0.8289104 | 1.0033842 |
| Zinc finger protein | 1.0469896 | 1.0496228 | 1.1140525 | 1.5749687 | 1.1378806 | 0.8914579 | 0.8872626 |
| c-myc | 1.0139033 | 0.9300892 | 1.4131157 | 1.1585134 | 1.0761367 | 1.2891656 | 1.5202777 |
| Cholesterol 7-alpha-hydroxylase (P450 | 0.871769 | 1.1795971 | 0.8969874 | 0.9553781 | 1.103954 | 1.2052213 | 1.0843471 |
| VII) | | | | | | | |
| Ferritin H-chain | 1.0051687 | 0.9027979 | 0.6966488 | 0.8457289 | 0.9041293 | 0.8913291 | 0.9314529 |
| Phase-1 RCT-197 | 1.0633507 | 1.6126094 | 1.0277888 | 1.8494068 | 1.7915419 | 1.1012408 | 1.0756879 |
| Phase-1 RCT-179 | 1.075666 | 1.0227627 | 0.9912298 | 1.4609925 | 1.1311903 | 0.9943913 | 0.981826 |
| Phase-1 RCT-15 | 1.1846255 | 1.0393164 | 1.3739927 | 2.1224775 | 1.502956 | 1.1832641 | 1.1794859 |
| Argininosuccinate lyase | 1.312659 | 0.9263911 | 1.3412305 | 1.2550868 | 1.0607688 | 1.3230845 | 1.2691153 |
| Phase-1 RCT-49 | 0.9660717 | 0.9643093 | 0.9078089 | 1.2817576 | 1.0872744 | 1.2430588 | 1.1278608 |
| Carbonic anhydrase III | 1.4908164 | 1.8716093 | 1 | 0.1475497 | 0.4866224 | 0.258654 | 0.3691317 |
| Proliferating cell nuclear antigen gene | 0.8601744 | 0.8769133 | 0.8530337 | 0.9578324 | 0.9547153 | 1.111214 | 1.1954437 |
| Integrin beta1 | 0.9519325 | 0.9054252 | 0.9437879 | 1.5262783 | 1.0779074 | 1.048401 | 0.9879161 |
| Caspase 6 | 1.007694 | 0.8921997 | 0.8859599 | 1.0158961 | 1.0174546 | 1.1193326 | 1.1750544 |
| Phase-1 RCT-116 | 1.153043 | 0.9717307 | 1.1790323 | 1.1322439 | 1.2059441 | 1.239121 | 1.081191 |
| Phase-1 RCT-109 | 0.8571022 | 0.8349381 | 0.9887829 | 1.1498079 | 0.9894577 | 1.129926 | 1.1585151 |
| Phase-1 RCT-71 | 0.9620208 | 1.0309701 | 1.0340916 | 1.035196 | 1.1684034 | 1.0356438 | 1.117964 |
| Elongation factor-1 alpha | 1.1261322 | 0.9616736 | 0.7631653 | 0.9606512 | 0.9494209 | 0.7079693 | 0.6838467 |
| Phase-1 RCT-169 | 1.1468896 | 0.9475765 | 0.9381145 | 1.1769083 | 1.1695924 | 1.1844944 | 1.2258343 |
| Pyruvate kinase, muscle | 1.0575631 | 0.8987153 | 0.8854221 | 1.2158251 | 1.1187308 | 1.1922996 | 1.2496331 |
| Nucleosome assembly protein | 0.8806569 | 1.069418 | 0.5712067 | 0.6767358 | 1.2068651 | 0.9061014 | 0.6762689 |
| Phase-1 RCT-127 | 0.9992068 | 0.9316971 | 0.9202188 | 1.4556537 | 1.0735358 | 1.1043243 | 1.3398445 |
| Iron-responsive element-binding protein | 0.8781023 | 1.0264262 | 0.8390865 | 0.7374175 | 0.8882994 | 0.9644662 | 0.847783 |
| Phase-1 RCT-72 | 0.9473144 | 0.9802647 | 0.9588726 | 1.029165 | 1.022861 | 1.0775236 | 1.2492676 |
| Phase-1 RCT-144 | 0.9804354 | 0.9543101 | 0.9457164 | 1.5438249 | 1.0996565 | 1.1699151 | 1.1627622 |
| Phase-1 RCT-242 | 0.9918053 | 1.0267856 | 1.0434762 | 1.4783349 | 1.2913167 | 1.2765857 | 1.2894098 |
| 17-beta hydroxysteroid dehydrogenase, | 0.8143635 | 0.9906108 | 0.2391497 | 0.1929986 | 1.2884898 | 0.7408887 | 0.5177307 |
| type 2 | | | | | | | |
| Phase-1 RCT-70 | 0.9665679 | 0.9805142 | 1.2780778 | 1.0346018 | 1.0664095 | 1.0861219 | 1.179101 |
| Ribosomal protein L13A | 0.9932134 | 0.7693387 | 0.9555017 | 1.2161046 | 1.0157439 | 1.210838 | 1.2890612 |
| Cytochrome P450 2E1 | 0.8136099 | 0.7925297 | 0.9490733 | 0.5908919 | 0.7514936 | 0.9112895 | 1.0095818 |
| AT-3 | 0.9886837 | 0.9622449 | 0.8272199 | 1.0712005 | 0.9653564 | 0.9079777 | 0.9871599 |
| Phase-1 RCT-270 | 1.0506384 | 1.2008718 | 0.9320362 | 0.3813088 | 0.7195416 | 0.7543777 | 0.8815535 |
| Phase-1 RCT-123 | 0.9786397 | 1.0819108 | 0.9672251 | 0.984696 | 0.9591073 | 1.1567732 | 1.1476073 |
| Alpha-2-macroglobulin, sequence 2 | 0.8834469 | 0.9661285 | 1.1263669 | 0.9698157 | 0.809572 | 0.985006 | 1.2280965 |
| Matrix metalloproteinase-1 | 1.0377477 | 0.9322615 | 0.7519623 | 1.0037076 | 0.998315 | 0.9920586 | 0.7480897 |
| Beta-tubulin, class I | 1.1028981 | 0.9777419 | 0.993758 | 1.1215174 | 1.1007712 | 1.1863842 | 1.1575985 |
| Phase-1 RCT-161 | 1.248987 | 1.5738437 | 1.0985065 | 0.5286984 | 0.7327712 | 0.4921234 | 0.7275832 |
| 14-3-3 zeta | 1.1617604 | 0.9144968 | 1.0831653 | 0.9453098 | 1.0466564 | 1.0548731 | 1.0185955 |
| Stem cell factor | 1.1137605 | 1.0290896 | 1.1043288 | 0.726838 | 0.9732372 | 0.7115492 | 0.6766106 |
| Macrophage inflammatory protein-2 alpha | 1.142998 | 0.9895675 | 1.3339353 | 1.7429882 | 1.1827828 | 1.1425744 | 1.2556816 |
| Thymidylate synthase | 0.9455715 | 0.9555261 | 0.9474168 | 1.0293771 | 1.0063599 | 1.3532429 | 1.2792171 |
| c-jun | 1.1732258 | 1.1112821 | 1.377724 | 1.4476137 | 1.6327779 | 1.1723542 | 1.1812063 |
| Superoxide dismutase Mn | 1.0701905 | 1.0922582 | 1.0634749 | 1.2846715 | 1.1412678 | 0.943814 | 1.0953131 |
| Phase-1 RCT-73 | 0.993381 | 1.0573082 | 0.8838857 | 0.9213241 | 0.9528358 | 0.8614791 | 0.7951661 |
| Macrophage inflammatory protein-1 alpha | 1.0375714 | 0.9426705 | 1.3448964 | 1.1044588 | 1.0287099 | 1.2771738 | 1.4294727 |
| Phase-1 RCT-214 | 1.169987 | 0.9232881 | 0.9444928 | 0.7866101 | 0.9548044 | 0.7892209 | 0.7302325 |
| NADH-cytochrome b5 reductase | 0.9184869 | 1.2695007 | 1.0919676 | 0.6755307 | 0.8003139 | 0.8233565 | 0.7699763 |
| Interleukin-1 beta | 1.0038328 | 0.9016447 | 1.0413157 | 1.1061457 | 1.0280035 | 1.0472503 | 1.0049618 |
| NADP-dependent isocitrate | 1.1088023 | 1.0472747 | 0.9119698 | 0.657993 | 0.8800879 | 0.8153426 | 0.8345487 |
| dehydrogenase, cytosolic | | | | | | | |
| Phase-1 RCT-289 | 1.0775697 | 1.3428202 | 1.1256331 | 0.7466352 | 0.9324309 | 0.7974989 | 0.7884184 |
| Glutamine synthetase | 1.0654241 | 0.8631264 | 0.9850354 | 0.7506993 | 0.9002107 | 0.4594576 | 0.6399438 |
| Phase-1 RCT-182 | 0.8926218 | 1.1424687 | 0.9568243 | 0.5835359 | 0.7529215 | 0.8545491 | 0.8938586 |
| Ubiquitin conjugating enzyme (RAD 6 | 1.0487088 | 0.9624461 | 1.0787557 | 1.0814711 | 1.0445465 | 0.7004555 | 0.9100702 |
| homologue) | | | | | | | |
| Extracellular-signal-regulated kinase 1 | 1.1473312 | 1.2304639 | 0.6675405 | 0.5497229 | 0.701839 | 0.4198536 | 0.5491192 |
| Emerin | 0.9541177 | 0.9228342 | 1.0399038 | 0.8749907 | 1.0325296 | 0.9655744 | 0.8577963 |
| Phase-1 RCT-78 | 1.202749 | 1.1721112 | 1.213186 | 0.9349142 | 1.0873504 | 0.9616714 | 0.9917502 |
| Phase-1 RCT-212 | 0.9105812 | 1.0936751 | 0.8627747 | 1.0068879 | 1.0500882 | 1.0894178 | 1.1343453 |
| Phase-1 RCT-168 | 1.0026037 | 1.0913441 | 1.0027974 | 0.6916419 | 0.7936948 | 0.6464934 | 0.704383 |
| Lysyl hydroxylase | 0.9800566 | 0.9951422 | 0.9527513 | 0.8757247 | 0.9750733 | 0.4567277 | 0.6147097 |
| Phase-1 RCT-55 | 0.8314361 | 0.7462573 | 0.9522511 | 0.7246615 | 0.7866244 | 0.9972466 | 1.4096879 |
| MHC class I antigen RT1.A1(f) alpha- | 1.6433688 | 0.9890818 | 1.8143188 | 1.4693921 | 1.5359718 | 1.2765774 | 1.0956408 |
| chain | | | | | | | |
| Phase-1 RCT-40 | 1.168917 | 1.1055082 | 0.7467327 | 0.7141311 | 0.915378 | 0.6844335 | 0.6250631 |
| Cyclin G | 1.1420199 | 1.022032 | 1.2676705 | 2.8617885 | 2.9459188 | 1.204908 | 1.0808052 |
| Peroxisome proliferator activated receptor | 1.0719354 | 0.9794838 | 0.9791546 | 1.0581055 | 1.0154052 | 1.2449276 | 1.1345785 |
| gamma | | | | | | | |
| Phase-1 RCT-280 | 1.0992044 | 1.0653718 | 1.1481214 | 0.4788407 | 1.2119995 | 1.0552299 | 0.9407668 |
| D-dopachrome tautomerase | 0.7970937 | 1.1284751 | 0.8457592 | 0.6180769 | 0.8395328 | 0.5432341 | 0.8098799 |
| Phase-1 RCT-287 | 1.0905342 | 1.106405 | 0.8173018 | 1.0597137 | 0.9243385 | 0.7745482 | 0.8412263 |
| Phase-1 RCT-119 | 1.1908362 | 1.1802434 | 1.1453332 | 0.7780972 | 0.8998249 | 1.2396293 | 1.1238561 |
| Thioredoxin-1 (Trx1) | 0.9557014 | 0.9115042 | 0.7677512 | 0.9110799 | 0.930181 | 0.7570159 | 0.9298024 |
| Phase-1 RCT-225 | 1.0230334 | 1.954339 | 0.6422282 | 1.3487167 | 1.7447056 | 1.5262644 | 1.3791325 |
| Protein kinase C alpha | 0.8729349 | 0.8879306 | 0.9711539 | 0.9008751 | 0.8914163 | 0.7052178 | 0.7676415 |
| Phase-1 RCT-87 | 1.0122727 | 1.0743634 | 0.8882371 | 0.8766595 | 0.9112563 | 0.9122974 | 0.9566474 |
| Phase-1 RCT-59 | 1.0430996 | 1.5573233 | 1.5939119 | 2.8676443 | 1.8918239 | 0.857369 | 1.029799 |
| C4b-binding protein | 1.2480739 | 0.9044602 | 0.5361842 | 1.1773075 | 1.1656041 | 0.6815159 | 0.7381374 |
| Phase-1 RCT-204 | 1.0505644 | 1.0294287 | 0.9552375 | 0.9700053 | 1.0281935 | 1.0282518 | 1.0849814 |
| Carnitine palmitoyl-CoA transferase | 0.980958 | 0.8556547 | 1.3096107 | 1.7319344 | 1.292786 | 1.3993397 | 1.1184275 |
| 8-oxoguanine DNA glycosylase | 0.9749963 | 0.9347489 | 0.9691103 | 1.001063 | 0.9723535 | 1.0602769 | 1.1446478 |
| H-rev107 | 1.0237635 | 0.9176257 | 0.9162567 | 0.8114615 | 0.8993862 | 1.0068452 | 1.0549963 |
| Preproalbumin | 1.3800415 | 1.2241707 | 1.1252767 | 1.0120108 | 1.0858369 | 1.0299516 | 1.4650089 |
| Phase-1 RCT-177 | 0.9784666 | 1.0600237 | 0.7317775 | 0.9190715 | 0.9551539 | 0.8155509 | 0.7971473 |
| Phase-1 RCT-110 | 1.0589597 | 0.9771525 | 0.9514105 | 1.0170902 | 0.9503539 | 1.2249172 | 1.1602638 |
| Phase-1 RCT-227 | 1.2583612 | 1.2580485 | 1.0751243 | 0.9631675 | 1.0757377 | 0.8646556 | 0.9926153 |
| Neuropeptide Y | 0.9315083 | 0.8811207 | 0.9718331 | 0.9115999 | 0.9296936 | 1.0631701 | 1.1607137 |
| Phase-1 RCT-277 | 0.8494698 | 0.6248229 | 0.8103315 | 0.7550037 | 0.9401246 | 1.0878145 | 0.89657 |
| Phase-1 RCT-229 | 0.9885305 | 0.9326184 | 0.9775661 | 0.9377722 | 0.9729041 | 1.1309967 | 1.1689243 |
| Phase-1 RCT-34 | 0.8913192 | 1.1544548 | 1.2713096 | 0.6516143 | 0.9061475 | 1.2220098 | 1.0768808 |
| Choline kinase | 1.0976198 | 0.9356477 | 1.3073691 | 1.204216 | 1.0679858 | 1.1104386 | 1.20285 |
| Phase-1 RCT-20 | 0.993942 | 0.9536305 | 1.0391593 | 1.1911302 | 1.0342994 | 1.1716925 | 1.2229816 |
| Phase-1 RCT-248 | 0.889097 | 0.9299191 | 1.0240046 | 1.1070495 | 0.9974635 | 1.2103872 | 1.2348486 |
| | APAP | APAP | APAP | APAP | AFLB | AFLB | AFLB |
| Compound-Dose (2) | 250 | 1000 | 1000 | 1000 | 1 | 1 | 1 |
| Animal Number (3) | 2123 | 2131 | 2132 | 2133 | 151 | 152 | 153 |
| Liver Toxicity Necrosis Classification (4) | no | yes | yes | yes | yes | yes | yes |
| Gene Name (5) | | | | | | | |
| Gadd153 | 1.2833676 | 2.8652697 | 2.2053692 | 16.526773 | 3.8833592 | 1.1859112 | 2.222405 |
| Interferon related developmental regulator | 0.8854412 | 3.0686626 | 4.0263376 | 9.338897 | 0.801223 | 0.8417943 | 1.0093904 |
| IFRD1 (PC4) | | | | | | | |
| ID-1 | 1.2572212 | 1.5057205 | 1.4065558 | 2.0600755 | 1.345004 | 1.3496997 | 1.3741778 |
| Insulin-like growth factor binding protein 1 | 1.0790439 | 8.173368 | 7.3543024 | 25.693027 | 1.2302867 | 1.1150937 | 1.2667177 |
| NIPK | 1.1935561 | 3.8341188 | 2.9738483 | 7.068734 | 1.4134004 | 1.1481261 | 1.4247699 |
| Phase-1 RCT-50 | 1.1992707 | 4.535991 | 2.916106 | 6.078297 | 2.374917 | 1.2867305 | 2.4035218 |
| Sarcoplasmic reticulum calcium ATPase | 1.1083212 | 1.1310183 | 1.2480335 | 1.0102949 | 0.6449171 | 0.800101 | 0.7092686 |
| Phase-1 RCT-207 | 1.3813392 | 4.0819297 | 4.76018 | 9.332855 | 2.1066828 | 1.8182721 | 2.1182115 |
| Acetyl-CoA carboxylase | 0.9753878 | 0.846795 | 0.972178 | 0.8962916 | 0.5719274 | 0.8692389 | 0.616393 |
| Cathepsin L, sequence 2 | 0.9983985 | 2.2613726 | 2.3919814 | 3.2431889 | 1.1740044 | 0.9799854 | 0.989967 |
| Glucokinase | 1.150218 | 0.6771162 | 0.6806226 | 0.5939342 | 0.4527957 | 0.9678382 | 0.5956947 |
| Gadd45 | 1.1867486 | 13.548119 | 10.54845 | 33.398987 | 2.0279684 | 1.2584152 | 1.7730714 |
| Phase-1 RCT-18 | 1.0468442 | 1.0412029 | 1.0545696 | 1.2883968 | 1.0513842 | 0.9758247 | 1.0705905 |
| 3-beta-hydroxysteroid dehydrogenase | 0.7573767 | 0.7013438 | 0.6695827 | 0.8355773 | 0.7048579 | 0.8104224 | 0.7591108 |
| (HSD3B1) | | | | | | | |
| Phase-1 RCT-221 | 1.0165893 | 0.9543521 | 1.1509932 | 1.0378331 | 1.0670931 | 0.9366294 | 1.0061761 |
| Heme oxygenase | 1.1400157 | 14.160956 | 13.905331 | 23.974522 | 1.2035546 | 0.9562836 | 1.1048781 |
| Cyclin dependent kinase 4 | 0.9668472 | 0.9148699 | 0.9529063 | 0.5247123 | 0.9831317 | 1.0286175 | 0.8530349 |
| Phase-1 RCT-75 | 0.9628602 | 1.0251853 | 0.9835197 | 0.852195 | 1.0655175 | 1.1846697 | 0.9762626 |
| Phase-1 RCT-65 | 1.3248705 | 1.7103213 | 1.9475437 | 3.0537066 | 1.2268347 | 1.1254463 | 1.2376106 |
| Cytochrome P450 1A1 | 1.3705655 | 0.988665 | 0.9345324 | 1.4325821 | 0.9220569 | 0.9819405 | 0.9526205 |
| Peroxisomal multifunctional enzyme type II | 0.7694708 | 0.9538303 | 0.8035187 | 0.5821927 | 0.9150877 | 0.9209869 | 0.846792 |
| DNA topoisomerase I | 0.4849266 | 0.3174208 | 0.511474 | 0.30757 | 0.8733667 | 0.8954018 | 0.9034812 |
| Focal adhesion kinase (pp125FAK) | 0.9491199 | 0.8118133 | 0.7950084 | 0.8085916 | 0.7406378 | 0.9552835 | 0.7383463 |
| NGF-inducible anti-proliferative putative | 0.6966537 | 4.662477 | 3.1785836 | 4.9241757 | 2.5086343 | 1.6741941 | 2.6233454 |
| secreted protein (PC3) | | | | | | | |
| Phase-1 RCT-82 | 1.4619927 | 0.9477491 | 1.0070275 | 1.249107 | 1.0706055 | 1.0376211 | 1.1337601 |
| Senescence marker protein-30 | 0.6322364 | 0.3392425 | 0.2538878 | 0.2065512 | 0.6044028 | 0.9323341 | 0.7914829 |
| Melanoma-associated antigen ME491 | 0.6266244 | 0.7820349 | 0.907084 | 0.9264899 | 1.2832224 | 1.0746332 | 1.3415979 |
| Activating transcription factor 3 | 1.1869835 | 5.2152505 | 4.37664 | 13.563021 | 1.253289 | 1.0005217 | 1.2484285 |
| Tryptophan hydroxylase | 0.9610753 | 0.79304 | 0.8511427 | 0.6003673 | 0.9653761 | 0.847791 | 0.9119945 |
| Zinc finger protein | 0.8689942 | 3.6127496 | 2.2939496 | 7.3033237 | 1.7115605 | 1.1299456 | 2.0643866 |
| c-myc | 1.4129616 | 3.5845058 | 3.6551895 | 13.280829 | 1.0013629 | 1.0964643 | 1.1761646 |
| Cholesterol 7-alpha-hydroxylase (P450 | 1.1924791 | 0.5289903 | 0.5434674 | 0.5297082 | 1.1266701 | 1 | 0.9255106 |
| VII) | | | | | | | |
| Ferritin H-chain | 1.0929906 | 1.4230838 | 1.6446908 | 1.820085 | 1.0360752 | 0.9749754 | 0.9652173 |
| Phase-1 RCT-197 | 1.0758157 | 2.1165779 | 2.3839889 | 3.551137 | 2.0215065 | 1.567097 | 1.9994967 |
| Phase-1 RCT-179 | 0.8602158 | 1.075475 | 1.0207168 | 1.5035985 | 1.0242791 | 1.1490386 | 0.9811522 |
| Phase-1 RCT-15 | 1.340549 | 1.7863206 | 1.7628686 | 2.3381824 | 1.1482751 | 1.2068318 | 1.2805529 |
| Argininosuccinate lyase | 1.336161 | 2.3801498 | 2.702852 | 2.5246582 | 0.9160447 | 1.2249084 | 0.9155895 |
| Phase-1 RCT-49 | 0.9920009 | 1.4747499 | 1.6550812 | 2.356271 | 1.6574907 | 1.2052785 | 1.6079613 |
| Carbonic anhydrase III | 0.2367094 | 0.2452198 | 0.2732631 | 0.3452185 | 0.5522181 | 0.5810199 | 0.4409009 |
| Proliferating cell nuclear antigen gene | 1.1269474 | 1.6552235 | 1.5776664 | 4.7867675 | 0.934554 | 0.934688 | 0.9995248 |
| Integrin beta1 | 1.2108377 | 9.811679 | 8.983649 | 14.055526 | 1.4203438 | 1.1860901 | 1.4045798 |
| Caspase 6 | 1.2268109 | 1.8303516 | 1.7658768 | 1.8847111 | 0.9884584 | 1.0559884 | 1.1180794 |
| Phase-1 RCT-116 | 1.335424 | 3.4295046 | 4.020015 | 3.05114 | 1.1939492 | 1.0487763 | 1.1342099 |
| Phase-1 RCT-109 | 1.1811211 | 1.103818 | 1.2927822 | 1.3460281 | 1.2342706 | 1.167453 | 1.2091409 |
| Phase-1 RCT-71 | 1.2409624 | 2.1761372 | 3.3321042 | 4.228915 | 0.8397183 | 0.9922937 | 0.8672417 |
| Elongation factor-1 alpha | 0.6238232 | 0.8366218 | 1.0346955 | 0.6222716 | 0.8776314 | 0.876751 | 0.8144835 |
| Phase-1 RCT-169 | 1.3380578 | 1.3189174 | 1.3159388 | 1.5242406 | 1.4181015 | 1.1180694 | 1.9467065 |
| Pyruvate kinase, muscle | 1.1756511 | 1.7246149 | 1.6991045 | 2.0649593 | 1.1494771 | 1.1204019 | 1.2236372 |
| Nucleosome assembly protein | 0.7705489 | 0.6281483 | 0.5161758 | 0.7318954 | 1.0073657 | 0.9001569 | 1.0688637 |
| Phase-1 RCT-127 | 1.1428993 | 2.67086 | 2.4201894 | 5.618094 | 1.5074401 | 1.154673 | 1.5562277 |
| Iron-responsive element-binding protein | 0.908981 | 0.9870875 | 0.7556651 | 0.4705097 | 0.835323 | 0.9370129 | 0.8326986 |
| Phase-1 RCT-72 | 1.2909082 | 1.0555958 | 1.0973085 | 1.1270634 | 0.996435 | 1.0046988 | 1.1549896 |
| Phase-1 RCT-144 | 1.1410645 | 1.3522149 | 1.3438575 | 1.9194092 | 1.5495565 | 1.254896 | 1.3619442 |
| Phase-1 RCT-242 | 1.3655989 | 11.27296 | 11.646449 | 14.282213 | 2.9460118 | 1.8133638 | 3.0279515 |
| 17-beta hydroxysteroid dehydrogenase, | 0.5392335 | 0.4039342 | 0.33889 | 0.2924891 | 0.9707062 | 0.684624 | 1.1185054 |
| type 2 | | | | | | | |
| Phase-1 RCT-70 | 1.2966 | 1.2999583 | 1.4533943 | 1.395045 | 1.2705626 | 1.0519055 | 1.1450558 |
| Ribosomal protein L13A | 1.254028 | 1.6759192 | 1.6898803 | 1.4947733 | 1.1337094 | 1.074133 | 0.993692 |
| Cytochrome P450 2E1 | 1.2940545 | 0.6245352 | 0.700478 | 1.3281869 | 0.5835394 | 1.0121319 | 0.9542792 |
| AT-3 | 0.9501555 | 0.77549 | 0.7471827 | 0.928106 | 1.0158845 | 0.9483119 | 0.998296 |
| Phase-1 RCT-270 | 0.8988545 | 0.4905311 | 0.5453829 | 0.4122464 | 0.5680312 | 0.866144 | 0.6855335 |
| Phase-1 RCT-123 | 1.2102956 | 1.2046596 | 1.1821133 | 1.6174935 | 1.0727158 | 1.0350996 | 1.1081657 |
| Alpha-2-macroglobulin, sequence 2 | 1.008187 | 1.8841972 | 2.5372458 | 2.5765734 | 0.9642751 | 0.9734716 | 0.9734989 |
| Matrix metalloproteinase-1 | 1.0687732 | 1.564284 | 1.6630691 | 1.5348325 | 1.2066159 | 1.0945992 | 1.2482721 |
| Beta-tubulin, class I | 1.5152087 | 2.134808 | 1.8188515 | 2.2801545 | 1.202307 | 1.6354953 | 1.2386514 |
| Phase-1 RCT-161 | 0.6703033 | 0.8583385 | 0.9613807 | 1.4579211 | 0.7440358 | 0.7527317 | 0.6286552 |
| 14-3-3 zeta | 1.5055902 | 1.2935524 | 1.274424 | 1.937108 | 1.0184702 | 1.1252446 | 1.0234483 |
| Stem cell factor | 0.7273573 | 0.7266302 | 0.6381391 | 0.3551296 | 0.741445 | 1.0372766 | 0.9011831 |
| Macrophage inflammatory protein-2 alpha | 1.1854746 | 1.7635239 | 1.9319863 | 2.937391 | 1.5103047 | 1.1140451 | 1.6523249 |
| Thymidylate synthase | 1.4898455 | 1.0538392 | 0.8344627 | 1.4778813 | 0.961453 | 0.9988866 | 0.9293894 |
| c-jun | 1.3416399 | 5.9796095 | 3.4196622 | 19.5575 | 2.084037 | 1.2743791 | 2.7992826 |
| Superoxide dismutase Mn | 1.1041778 | 1.1113485 | 1.099426 | 1.2033713 | 1.1368082 | 1.04943 | 1.1317236 |
| Phase-1 RCT-73 | 0.7951185 | 0.7932463 | 0.7592893 | 0.6148349 | 0.8974472 | 0.9713426 | 0.8946707 |
| Macrophage inflammatory protein-1 alpha | 1.3512194 | 1.2765998 | 1.3482242 | 1.6690646 | 1.2612367 | 1.0736232 | 1.4281094 |
| Phase-1 RCT-214 | 0.7106905 | 0.5689397 | 0.4893059 | 0.5038679 | 0.5124366 | 1.0846788 | 0.5194139 |
| NADH-cytochrome b5 reductase | 0.8176254 | 0.4992453 | 0.6535707 | 0.4960838 | 0.7335173 | 0.9519077 | 0.7786831 |
| Interleukin-1 beta | 1.1580855 | 1.2193122 | 1.3650312 | 1.4973493 | 0.8821446 | 0.9891229 | 0.9040233 |
| NADP-dependent isocitrate | 0.7464656 | 0.613383 | 0.5675144 | 0.5276151 | 0.6363973 | 0.8949876 | 0.5124672 |
| dehydrogenase, cytosolic | | | | | | | |
| Phase-1 RCT-289 | 0.8429475 | 0.5924577 | 0.6081959 | 0.6202735 | 0.7536095 | 0.8116719 | 0.7709159 |
| Glutamine synthetase | 0.4982349 | 0.3969865 | 0.3727181 | 0.3012313 | 0.9870188 | 0.8643474 | 0.9600273 |
| Phase-1 RCT-182 | 0.8819477 | 0.4880117 | 0.5739881 | 0.4716944 | 0.6012949 | 0.7359619 | 0.7785707 |
| Ubiquitin conjugating enzyme (RAD 6 | 0.6427807 | 0.8069672 | 0.7713166 | 1.0204965 | 0.9152776 | 1.0339141 | 0.9855464 |
| homologue) | | | | | | | |
| Extracellular-signal-regulated kinase 1 | 0.4436902 | 0.7546413 | 0.6381694 | 0.3511591 | 0.9250814 | 1.0083622 | 0.8986571 |
| Emerin | 1.0044427 | 0.9028459 | 0.8096917 | 0.8449364 | 1.0125816 | 1.0449198 | 1.03981 |
| Phase-1 RCT-78 | 0.882611 | 0.7213889 | 0.8645054 | 0.9121452 | 0.880646 | 0.9859061 | 1.0141305 |
| Phase-1 RCT-212 | 1.129727 | 1.0167435 | 0.9820968 | 1.0684634 | 0.8705843 | 0.9893755 | 0.8246544 |
| Phase-1 RCT-168 | 0.6684185 | 0.5713901 | 0.5481381 | 0.4552002 | 0.5115341 | 1.0012994 | 0.6087013 |
| Lysyl hydroxylase | 0.4928167 | 0.4412355 | 0.471529 | 0.3402851 | 1.0047185 | 0.9592797 | 0.9796999 |
| Phase-1 RCT-55 | 0.9905435 | 1.232559 | 1.0003196 | 0.7491241 | 1.043981 | 1.0839958 | 1.1921021 |
| MHC class I antigen RT1.A1(f) alpha- | 1.2988966 | 1.6591353 | 1.8104838 | 1.9732566 | 1.3480786 | 1.1661986 | 1.389407 |
| chain | | | | | | | |
| Phase-1 RCT-40 | 0.6127362 | 0.5150169 | 0.6354469 | 0.3835705 | 0.737513 | 0.8609299 | 0.8431448 |
| Cyclin G | 1.218994 | 2.001303 | 1.7833366 | 3.6087186 | 3.858344 | 2.4275937 | 3.877533 |
| Peroxisome proliferator activated receptor | 1.5170406 | 1.269258 | 0.9505113 | 1.2365546 | 1.1919423 | 1.0771556 | 1.0876944 |
| gamma | | | | | | | |
| Phase-1 RCT-280 | 0.9296427 | 0.747173 | 0.5944901 | 0.9856027 | 0.9494678 | 1.0272999 | 1.0607578 |
| D-dopachrome tautomerase | 0.7741816 | 0.4240231 | 0.566384 | 0.5321072 | 0.6818873 | 0.8673754 | 0.6344342 |
| Phase-1 RCT-287 | 0.7807378 | 0.7618116 | 0.6975372 | 0.5842825 | 1.0164065 | 0.9006732 | 0.8372665 |
| Phase-1 RCT-119 | 1.575786 | 0.680642 | 0.9481295 | 2.119288 | 0.8064894 | 0.8496223 | 0.8126093 |
| Thioredoxin-1 (Trx1) | 0.8285027 | 0.8585535 | 0.8826543 | 1.0021559 | 0.9934167 | 1.0377915 | 1.1050999 |
| Phase-1 RCT-225 | 1.1217961 | 0.9779037 | 0.8794202 | 1.284452 | 1.2959278 | 0.9439515 | 1.0442888 |
| Protein kinase C alpha | 0.6613561 | 0.7075053 | 0.7613173 | 0.4578037 | 0.9341098 | 0.9663448 | 0.9204977 |
| Phase-1 RCT-87 | 1.1432753 | 0.6521053 | 0.6556129 | 0.6937014 | 0.7346196 | 0.9817095 | 0.714859 |
| Phase-1 RCT-59 | 0.9008778 | 2.4023085 | 3.3261206 | 3.3207152 | 3.4990766 | 2.2734041 | 3.0521646 |
| C4b-binding protein | 0.7397209 | 0.4105776 | 0.5429909 | 0.4467945 | 0.8031535 | 0.9076666 | 1.0161415 |
| Phase-1 RCT-204 | 1.1127936 | 1.5019838 | 1.5661345 | 2.150679 | 1.3204478 | 1.1289903 | 1.3324735 |
| Carnitine palmitoyl-CoA transferase | 1.2132612 | 1.9683046 | 2.0227141 | 2.8518858 | 1.2222464 | 1.1607535 | 1.1837156 |
| 8-oxoguanine DNA glycosylase | 1.1321105 | 0.937788 | 0.9086896 | 0.8296641 | 0.9379517 | 0.9618533 | 1.0034939 |
| H-rev107 | 1.0920812 | 0.7136611 | 0.8658882 | 0.9965694 | 0.9857026 | 1.0195576 | 1.1099597 |
| Preproalbumin | 1.1069688 | 1.6604996 | 1.7491046 | 1.480588 | 1.011479 | 0.932837 | 0.803871 |
| Phase-1 RCT-177 | 0.7448074 | 0.6922967 | 0.6148238 | 0.4757334 | 0.8072098 | 0.8956054 | 0.7880521 |
| Phase-1 RCT-110 | 1.3190069 | 1.0537738 | 1.0217078 | 1.6215957 | 1.1138569 | 1.0275468 | 1.0956928 |
| Phase-1 RCT-227 | 0.7902363 | 0.7560682 | 0.9408557 | 1.0318712 | 1.0470771 | 0.8737387 | 1.0100106 |
| Neuropeptide Y | 1.1003243 | 1.0657674 | 1.0751232 | 1.1736677 | 1.1800393 | 1.0212678 | 1.1195029 |
| Phase-1 RCT-277 | 1.0258058 | 0.4458227 | 0.6084315 | 0.4691939 | 0.8598763 | 0.9803214 | 0.8754364 |
| Phase-1 RCT-229 | 1.2298563 | 1.1051877 | 1.1345339 | 1.4589843 | 1.1055484 | 0.9956344 | 1.1371984 |
| Phase-1 RCT-34 | 1.3125105 | 1.643493 | 1.9507734 | 2.344811 | 0.7083609 | 1.1385628 | 0.8522291 |
| Choline kinase | 1.409967 | 2.7410498 | 3.1735117 | 5.967005 | 1.605774 | 1.3762556 | 1.3088061 |
| Phase-1 RCT-20 | 1.2796857 | 1.3857325 | 1.4120107 | 1.5395861 | 1.0600735 | 1.0839695 | 1.0870066 |
| Phase-1 RCT-248 | 1.2519598 | 1.0471131 | 1.2127873 | 1.4854348 | 1.1022316 | 1.0706614 | 1.1607368 |
| | AMPB | AMPB | AMPB | AMPB | AMPB | AMPB | AZA |
| Compound-Dose (2) | 5 | 5 | 5 | 20 | 20 | 20 | 50 |
| Animal Number (3) | 441 | 442 | 443 | 451 | 452 | 453 | 1821 |
| Liver Toxicity Necrosis Classification (4) | no | no | no | no | no | no | no |
| Gene Name (5) | | | | | | | |
| Gadd153 | 1.2815554 | 2.22445 | 1.2827231 | 1.6263807 | 1.1231719 | 1.2818065 | 1.3781147 |
| Interferon related developmental regulator | 1.0884724 | 1.2428341 | 0.9731662 | 1.1907465 | 0.8377063 | 1.276896 | 1.3465952 |
| IFRD1 (PC4) | | | | | | | |
| ID-1 | 0.9960418 | 0.8029466 | 1.0505867 | 0.8958256 | 1.0225111 | 0.8994235 | 1.6640673 |
| Insulin-like growth factor binding protein 1 | 1.142369 | 1.1695325 | 0.8681027 | 1.9246669 | 0.99709 | 1.2503277 | 1.8425959 |
| NIPK | 0.8721312 | 0.7416424 | 0.8195843 | 0.9686022 | 0.5280728 | 0.9487355 | 1.3648316 |
| Phase-1 RCT-50 | 0.8621526 | 0.9166466 | 1.004528 | 1.0540372 | 0.8991864 | 0.949495 | 1.3452727 |
| Sarcoplasmic reticulum calcium ATPase | 1.11326 | 1.2759304 | 1.2022578 | 1.1440561 | 0.9110837 | 1.3080571 | 1.384101 |
| Phase-1 RCT-207 | 0.8795347 | 0.9616903 | 0.9055985 | 0.9986825 | 0.8465316 | 0.967033 | 1.7038776 |
| Acetyl-CoA carboxylase | 0.8252425 | 0.7076232 | 0.690578 | 0.9132897 | 0.7484036 | 0.9811739 | 0.9299278 |
| Cathepsin L, sequence 2 | 1.6617501 | 1.6574585 | 1.1471232 | 0.9896577 | 1.9307734 | 0.9417703 | 1.8508434 |
| Glucokinase | 0.9605427 | 0.9508268 | 1.2693534 | 0.7507696 | 1.0961038 | 0.9420203 | 1.2714854 |
| Gadd45 | 1.6208329 | 1.9381377 | 1.7596792 | 1.3863268 | 0.9190062 | 1.1223677 | 0.9838989 |
| Phase-1 RCT-18 | 0.8642171 | 0.8515908 | 0.8717668 | 0.8827526 | 0.7767737 | 0.9760619 | 0.9811609 |
| 3-beta-hydroxysteroid dehydrogenase | 1.2955503 | 1.2154696 | 0.7852871 | 1.176074 | 0.9469906 | 0.6587974 | 0.9125461 |
| (HSD3B1) | | | | | | | |
| Phase-1 RCT-221 | 0.9182245 | 0.7301281 | 0.7376878 | 0.6548318 | 0.5639867 | 0.8387592 | 1.0619041 |
| Heme oxygenase | 1.3190682 | 1.8091581 | 1.3499751 | 1.2124782 | 0.9913346 | 1.0430529 | 1.3504897 |
| Cyclin dependent kinase 4 | 1.3632787 | 1.4305674 | 1.1181933 | 1.2661705 | 1.1126167 | 1.2121212 | 1.5806998 |
| Phase-1 RCT-75 | 0.9485362 | 0.811941 | 0.7282997 | 0.8759757 | 0.7611266 | 0.8644222 | 1.8957809 |
| Phase-1 RCT-65 | 0.7746588 | 0.7258351 | 0.8514756 | 1.0328242 | 0.8360089 | 0.9434525 | 1.6876643 |
| Cytochrome P450 1A1 | 1.031593 | 0.9696133 | 1.2418033 | 0.9481726 | 0.9409009 | 1.2993199 | 0.8563763 |
| Peroxisomal multifunctional enzyme type II | 1.4486805 | 1.4581461 | 1.1011746 | 1.0035414 | 1.4129606 | 0.8995599 | 1.7165074 |
| DNA topoisomerase I | 2.3251815 | 2.4081612 | 1.3893541 | 1.0614594 | 1.1225266 | 1.5506701 | 1.6180353 |
| Focal adhesion kinase (pp125FAK) | 1.4233799 | 1.5486678 | 1.3978257 | 1.0656973 | 1.2227253 | 1.0257143 | 1.3348763 |
| NGF-inducible anti-proliferative putative | 0.8824803 | 0.9914199 | 0.8134917 | 1.1299344 | 0.8117443 | 1.042277 | 1.9652187 |
| secreted protein (PC3) | | | | | | | |
| Phase-1 RCT-82 | 0.8359786 | 0.856136 | 0.8365833 | 0.9389535 | 0.8374729 | 0.9958392 | 1.0019519 |
| Senescence marker protein-30 | 1.3086267 | 1.5239292 | 0.8273349 | 0.869179 | 1.223318 | 1.1914744 | 0.5689768 |
| Melanoma-associated antigen ME491 | 0.9772607 | 1.085368 | 1.1645416 | 0.9030044 | 1.1117862 | 0.9829512 | 1.2925025 |
| Activating transcription factor 3 | 0.8046288 | 0.6905121 | 0.9192277 | 1.095002 | 0.6723657 | 0.9440995 | 1.2245198 |
| Tryptophan hydroxylase | 0.9051482 | 0.8991296 | 0.779321 | 0.8379424 | 0.8766744 | 1.0684847 | 1.0110428 |
| Zinc finger protein | 0.8390991 | 0.8902432 | 0.7675603 | 0.8484161 | 0.9692287 | 0.9505841 | 1.4017214 |
| c-myc | 0.9695715 | 1.002009 | 1.2973942 | 1.0542092 | 0.9083367 | 1.0170882 | 1.1329833 |
| Cholesterol 7-alpha-hydroxylase (P450 | 1.0557377 | 0.7398098 | 1.3561798 | 0.9445673 | 0.9291052 | 0.9341318 | 0.8695393 |
| VII) | | | | | | | |
| Ferritin H-chain | 1.9159555 | 2.2409635 | 1.1695658 | 1.0052587 | 1.8420279 | 0.7052673 | 0.8699903 |
| Phase-1 RCT-197 | 0.9329673 | 1.0369061 | 1.0211911 | 1.1739801 | 0.8228021 | 1.0974634 | 1.5864809 |
| Phase-1 RCT-179 | 1.7008828 | 2.481638 | 1.4084326 | 1.6958723 | 1.0987834 | 2.1671374 | 1.9186268 |
| Phase-1 RCT-15 | 1.5342574 | 1.4600675 | 1.0381784 | 1.2275982 | 1.166384 | 0.7835397 | 1.595185 |
| Argininosuccinate lyase | 0.8976321 | 0.8004414 | 0.6035886 | 0.9074159 | 1.198802 | 1.1194417 | 3.245213 |
| Phase-1 RCT-49 | 0.9808715 | 0.9731344 | 1.1293201 | 0.8719382 | 0.9526922 | 1.0402219 | 1.023024 |
| Carbonic anhydrase III | 0.6573786 | 0.62407 | 0.457007 | 0.2938376 | 1.1426109 | 0.7471356 | 0.2275264 |
| Proliferating cell nuclear antigen gene | 1 | 1.067941 | 1.281485 | 1.1246222 | 0.931118 | 1.1586571 | 1.5951552 |
| Integrin beta1 | 0.8470589 | 0.7886362 | 0.9811975 | 1.4811423 | 0.8070595 | 1.0812325 | 1.2374202 |
| Caspase 6 | 0.9848758 | 1.0501101 | 1.0324492 | 1.184546 | 0.8532372 | 1.0167674 | 1.493948 |
| Phase-1 RCT-116 | 0.6646635 | 0.6434578 | 0.574877 | 1.2544726 | 0.6801186 | 0.8806511 | 1.7654039 |
| Phase-1 RCT-109 | 1.0242515 | 1.0495168 | 0.8084617 | 1.028897 | 0.7603701 | 1.0727433 | 1.3078041 |
| Phase-1 RCT-71 | 0.8593702 | 0.8316996 | 0.8483239 | 0.9863653 | 0.8059375 | 0.9783551 | 1.424407 |
| Elongation factor-1 alpha | 2.1465073 | 2.2840316 | 1.2199965 | 1.0976102 | 1.4771649 | 0.998695 | 1.6447343 |
| Phase-1 RCT-169 | 0.8890423 | 0.9568057 | 1.1000484 | 1.6461843 | 0.723598 | 2.2146275 | 0.6901292 |
| Pyruvate kinase, muscle | 0.9829668 | 0.9290081 | 1.0119042 | 1.0804341 | 0.9930127 | 0.9387756 | 0.9379457 |
| Nucleosome assembly protein | 0.8753691 | 0.8616391 | 0.9330908 | 0.8687897 | 1.125568 | 1.0341239 | 0.9215828 |
| Phase-1 RCT-127 | 1.0991471 | 1.2588633 | 1.1400969 | 0.9542866 | 0.8613882 | 1.0906937 | 1.5926666 |
| Iron-responsive element-binding protein | 0.9877482 | 0.8936464 | 0.9399031 | 0.8975889 | 1.1180358 | 1.0172685 | 1.131405 |
| Phase-1 RCT-72 | 0.8106403 | 1.4478388 | 1.0226473 | 1.1521875 | 0.8004521 | 1.0341616 | 1.0191278 |
| Phase-1 RCT-144 | 1.3629977 | 1.6870812 | 1.57457 | 1.158573 | 1.0156773 | 1.2654474 | 2.6620753 |
| Phase-1 RCT-242 | 0.9815138 | 1.0721933 | 1.2027814 | 1.1666648 | 0.9138585 | 1.0666667 | 1.506152 |
| 17-beta hydroxysteroid dehydrogenase, | 0.8135538 | 0.881378 | 1.0169827 | 0.9081136 | 2.0692112 | 0.919673 | 0.4092393 |
| type 2 | | | | | | | |
| Phase-1 RCT-70 | 0.6742475 | 0.5959605 | 0.6082587 | 0.8551307 | 0.6213722 | 0.8886092 | 1.0267142 |
| Ribosomal protein L13A | 1.1706169 | 1.1926796 | 0.8330715 | 1.1161859 | 0.7175542 | 1.307635 | 1.5213656 |
| Cytochrome P450 2E1 | 1.2500467 | 1.2928176 | 1.2680922 | 0.7565138 | 1.1402076 | 0.9826303 | 0.9256686 |
| AT-3 | 1.207276 | 1.2777036 | 1.2967584 | 0.9998786 | 1.7388157 | 0.8945578 | 0.8295817 |
| Phase-1 RCT-270 | 0.7475411 | 0.3789617 | 0.5332491 | 0.5383752 | 0.7234679 | 0.841639 | 0.5997061 |
| Phase-1 RCT-123 | 0.9704918 | 0.9192628 | 1.0474521 | 0.9943767 | 1.0256511 | 1.0416667 | 0.8451823 |
| Alpha-2-macroglobulin, sequence 2 | 0.8671539 | 0.8802449 | 0.9053454 | 1.25346 | 1.0107871 | 1.5569087 | 1.23391 |
| Matrix metalloproteinase-1 | 1.4380375 | 1.5708599 | 1.1139706 | 1.0305166 | 1.5960741 | 0.7094278 | 1.8254836 |
| Beta-tubulin, class I | 1.0567042 | 1.1779519 | 0.958498 | 1.2239285 | 1.1280898 | 0.7142857 | 3.0896769 |
| Phase-1 RCT-161 | 0.8584848 | 0.8267794 | 0.9350957 | 0.7729408 | 0.8613883 | 0.8489124 | 0.4772063 |
| 14-3-3 zeta | 1.0919333 | 1.1656483 | 1.1036241 | 1.123189 | 1.0312692 | 0.9885163 | 1.3275582 |
| Stem cell factor | 1.0001287 | 1.1375046 | 0.9236532 | 1.0290661 | 1.0564723 | 1.1428571 | 0.9192463 |
| Macrophage inflammatory protein-2 alpha | 1.3236535 | 1.9178009 | 1.3106743 | 1.599325 | 1.0535439 | 1.1626041 | 1.444545 |
| Thymidylate synthase | 0.9933322 | 0.9342039 | 1.1048442 | 1.013651 | 0.9252456 | 0.9882354 | 0.9671611 |
| c-jun | 0.9031206 | 0.909069 | 1.1666566 | 1.3716072 | 0.7587181 | 0.9435775 | 1.9331286 |
| Superoxide dismutase Mn | 2.8455303 | 6.6514874 | 2.055473 | 5.7094407 | 0.8716567 | 3.1926608 | 1.2504618 |
| Phase-1 RCT-73 | 0.9584988 | 0.9044674 | 0.9066992 | 0.9257829 | 1.1669708 | 0.9054327 | 1.0115399 |
| Macrophage inflammatory protein-1 alpha | 1.014009 | 1.5515178 | 1.6593884 | 3.4168303 | 0.9196251 | 4.1036663 | 1.0827726 |
| Phase-1 RCT-214 | 0.8298774 | 0.8245065 | 0.7530702 | 0.6947228 | 0.9738001 | 0.9269555 | 1.1232727 |
| NADH-cytochrome b5 reductase | 0.724403 | 0.518932 | 0.5604603 | 0.6337785 | 0.8715504 | 0.9169055 | 0.9202379 |
| Interleukin-1 beta | 1.6692352 | 1.9413327 | 1.206328 | 1.8556786 | 1.5662885 | 1.4918212 | 1.3826402 |
| NADP-dependent isocitrate | 0.8660197 | 0.7330703 | 0.5779246 | 0.8402528 | 1.1019773 | 0.9003602 | 0.8779755 |
| dehydrogenase, cytosolic | | | | | | | |
| Phase-1 RCT-289 | 1.005513 | 0.7725764 | 0.6248746 | 0.8313635 | 1.1866156 | 1.0164912 | 0.9034695 |
| Glutamine synthetase | 1.8060124 | 1.8759016 | 1.4036244 | 0.9213638 | 1.4577339 | 1.3041193 | 1.7682787 |
| Phase-1 RCT-182 | 0.9790534 | 0.8870774 | 0.9079347 | 0.6062654 | 1.1961141 | 1.0157619 | 0.8733454 |
| Ubiquitin conjugating enzyme (RAD 6 | 1.682641 | 2.6777277 | 1.7114688 | 1.3161446 | 1.3883181 | 1.0068028 | 1.1597718 |
| homologue) | | | | | | | |
| Extracellular-signal-regulated kinase 1 | 0.7513485 | 0.6454964 | 0.6782128 | 0.6708033 | 1.0605838 | 0.9008842 | 1.2373748 |
| Emerin | 0.8274536 | 0.7654445 | 0.7690964 | 0.8400379 | 0.7695857 | 0.8571429 | 1.1213628 |
| Phase-1 RCT-78 | 0.8078689 | 0.7323261 | 0.7542073 | 0.7054814 | 0.8544861 | 0.8413216 | 0.9309174 |
| Phase-1 RCT-212 | 0.862872 | 1.0157853 | 0.97511 | 1.0545986 | 0.8246079 | 1.1156462 | 1.3270696 |
| Phase-1 RCT-168 | 1.137693 | 0.9326424 | 0.8516197 | 0.8681976 | 1.2486448 | 0.8638769 | 0.7994278 |
| Lysyl hydroxylase | 1.5996984 | 1.8760462 | 1.4870536 | 0.9972433 | 1.464436 | 1.4476191 | 1.3971894 |
| Phase-1 RCT-55 | 0.7959092 | 0.8698116 | 1.027643 | 0.9120147 | 1.1281852 | 0.9927585 | 1.0631729 |
| MHC class I antigen RT1.A1(f) alpha- | 0.661409 | 0.4987175 | 0.6462881 | 1.2530849 | 0.6170302 | 0.7750866 | 1.3714314 |
| chain | | | | | | | |
| Phase-1 RCT-40 | 0.9208795 | 0.9553725 | 0.6295013 | 0.8417652 | 0.8418249 | 0.7860583 | 0.9231428 |
| Cyclin G | 1.5188445 | 2.003793 | 1.3158327 | 1.8473654 | 1.0757948 | 1.2298648 | 2.7765043 |
| Peroxisome proliferator activated receptor | 0.9899165 | 0.899432 | 0.9780455 | 1.0186231 | 1.0364383 | 1.0749035 | 0.8753398 |
| gamma | | | | | | | |
| Phase-1 RCT-280 | 1.0481311 | 1.1514975 | 0.743403 | 0.9837706 | 1.0749662 | 1.0313076 | 0.7827933 |
| D-dopachrome tautomerase | 1.5270028 | 1.3458848 | 1.3664465 | 0.7981592 | 1.6320134 | 0.7127796 | 1.7746048 |
| Phase-1 RCT-287 | 1.0146679 | 1.2017545 | 0.8914678 | 0.9759933 | 1.1189647 | 0.9604863 | 1.3184599 |
| Phase-1 RCT-119 | 0.618373 | 0.6522032 | 0.685531 | 0.7172119 | 0.6587086 | 0.8814782 | 0.8668115 |
| Thioredoxin-1 (Trx1) | 1.0304661 | 1.2193178 | 0.8520268 | 1.1992112 | 0.7804762 | 1.1848342 | 1.4049199 |
| Phase-1 RCT-225 | 0.623296 | 0.7486572 | 0.670354 | 1.197863 | 0.5803517 | 1.207283 | 2.154617 |
| Protein kinase C alpha | 1.147541 | 1.4439099 | 1.3066121 | 0.9774463 | 1.0812331 | 0.9247547 | 1.0655121 |
| Phase-1 RCT-87 | 0.9923328 | 1.0700049 | 0.9046232 | 0.9407218 | 0.9710311 | 1.0277023 | 1.7660452 |
| Phase-1 RCT-59 | 0.9563984 | 0.9986361 | 1.0126835 | 0.9857324 | 1.0434306 | 0.9149561 | 1.8286296 |
| C4b-binding protein | 1.7961113 | 2.2868807 | 1.3581133 | 1.2151393 | 1.1341035 | 1.1361027 | 0.6853663 |
| Phase-1 RCT-204 | 0.9473242 | 0.9503516 | 0.9016564 | 0.9474008 | 0.9855861 | 1.0119572 | 1.0174831 |
| Carnitine palmitoyl-CoA transferase | 0.9479222 | 0.9730886 | 1.2914367 | 1.0168656 | 0.9122443 | 1.0126337 | 3.2076786 |
| 8-oxoguanine DNA glycosylase | 1.0531216 | 1.0174766 | 1.2601582 | 0.9219414 | 0.9065659 | 1.0679612 | 0.9989176 |
| H-rev107 | 1.0527982 | 0.9817797 | 1.0289665 | 1.0200597 | 1.0260999 | 1.0261781 | 0.8171401 |
| Preproalbumin | 1.0922517 | 0.9998509 | 0.4436021 | 1.163583 | 1.2411113 | 0.6309384 | 0.9063754 |
| Phase-1 RCT-177 | 1.0467489 | 1.0233972 | 0.6481074 | 0.8724402 | 0.9526593 | 0.8066127 | 1.3214151 |
| Phase-1 RCT-110 | 0.9358587 | 0.9305552 | 1.0806825 | 0.9086849 | 1.0540538 | 0.9475952 | 0.7626218 |
| Phase-1 RCT-227 | 0.9733071 | 1.0201755 | 0.8235298 | 0.6616358 | 1.1997972 | 0.8174468 | 0.8849589 |
| Neuropeptide Y | 0.9721841 | 0.9891285 | 1.1028893 | 0.9963159 | 1.1013198 | 0.9544742 | 0.8040733 |
| Phase-1 RCT-277 | 1.2602087 | 1.3503413 | 0.765976 | 1.3098913 | 1.4923527 | 1.1787853 | 0.9559842 |
| Phase-1 RCT-229 | 0.8300371 | 0.8910498 | 0.9759458 | 0.9396569 | 0.8816939 | 0.8749145 | 0.8034117 |
| Phase-1 RCT-34 | 0.7865872 | 0.6976745 | 0.9467362 | 1.0304617 | 0.8415674 | 1.0398529 | 1.8435344 |
| Choline kinase | 0.7532733 | 0.6464502 | 0.974058 | 1.1024599 | 0.8084133 | 1.2106538 | 1.680069 |
| Phase-1 RCT-20 | 0.9334688 | 0.9343202 | 1.0333788 | 1.1269286 | 0.937657 | 0.9882678 | 1.1772666 |
| Phase-1 RCT-248 | 0.9365998 | 0.9468414 | 0.9399359 | 0.9812439 | 0.7951276 | 0.938945 | 0.883486 |
| | AZA | AZA | AZA | AZA | AZA | BEN | BEN |
| Compound-Dose (2) | 50 | 50 | 200 | 200 | 200 | 250 | 250 |
| Animal Number (3) | 1822 | 1823 | 1831 | 1832 | 1833 | 2021 | 2022 |
| Liver Toxicity Necrosis Classification (4) | no | no | no | no | no | no | no |
| Gene Name (5) | | | | | | | |
| Gadd153 | 1.8454348 | 1.6153513 | 2.2703047 | 1.2577282 | 3.630317 | 0.7684429 | 1.1583636 |
| Interferon related developmental regulator | 1.2087835 | 1.1382556 | 1.3226651 | 1.1391714 | 1.674888 | 1.2101073 | 0.9631772 |
| IFRD1 (PC4) | | | | | | | |
| ID-1 | 2.0393023 | 1.7899401 | 1.3756752 | 1.2945112 | 1.0365846 | 0.8753756 | 1.2192289 |
| Insulin-like growth factor binding protein 1 | 1.4774516 | 0.9600517 | 4.1866317 | 4.025772 | 12.340709 | 1.2612039 | 0.9549934 |
| NIPK | 1.5692705 | 2.1763103 | 1.8816975 | 1.2518861 | 2.5278463 | 1.1369338 | 1.2889326 |
| Phase-1 RCT-50 | 2.3932905 | 2.7417428 | 2.815239 | 1.8633687 | 4.198423 | 1.0228636 | 1.0317639 |
| Sarcoplasmic reticulum calcium ATPase | 1.237868 | 1.111254 | 1.1861162 | 0.9933422 | 1.452521 | 1.1671053 | 0.9653907 |
| Phase-1 RCT-207 | 1.2298633 | 1.4988973 | 1.4156235 | 1.170791 | 2.1685083 | 1.0301977 | 1.2152554 |
| Acetyl-CoA carboxylase | 0.9585775 | 1.0783294 | 0.8387573 | 1.0194608 | 1.0003153 | 0.9302827 | 0.942078 |
| Cathepsin L, sequence 2 | 1.4150711 | 1.5586997 | 2.1377058 | 1.3695593 | 2.4141994 | 1.3124033 | 0.9678798 |
| Glucokinase | 1.3804253 | 1.5879945 | 1.2336694 | 1.3445233 | 1.3331839 | 0.915666 | 0.8561947 |
| Gadd45 | 0.8941425 | 1.1490391 | 1.2975433 | 1.0828297 | 1.6210274 | 0.9032624 | 1.22626 |
| Phase-1 RCT-18 | 0.9620071 | 1.0106235 | 1.0081054 | 1.0918618 | 1.128888 | 0.9678527 | 0.9876105 |
| 3-beta-hydroxysteroid dehydrogenase | 0.9045486 | 1.0175962 | 1.004205 | 0.9554967 | 0.9176639 | 1.176781 | 0.9023594 |
| (HSD3B1) | | | | | | | |
| Phase-1 RCT-221 | 1.0077071 | 1.0987163 | 1.1576494 | 1.0550746 | 1.2389913 | 0.8856884 | 0.8765698 |
| Heme oxygenase | 3.287646 | 1.7216042 | 3.0181582 | 1.8490497 | 3.2434382 | 1.0135045 | 1.1904697 |
| Cyclin dependent kinase 4 | 1.4084543 | 1.1774492 | 0.9712563 | 1.1932667 | 1.151295 | 1.2534981 | 0.9286539 |
| Phase-1 RCT-75 | 1.9019328 | 2.4566526 | 1.6907684 | 1.5872147 | 2.0907946 | 0.9638478 | 0.9611864 |
| Phase-1 RCT-65 | 2.0520449 | 2.195232 | 1.4784111 | 1.6742237 | 1.7555109 | 0.9429258 | 1.0514684 |
| Cytochrome P450 1A1 | 0.898658 | 0.9186718 | 1.0645486 | 1.0787554 | 1.1019984 | 0.8286161 | 1.3541065 |
| Peroxisomal multifunctional enzyme type II | 1.4914567 | 1.1491429 | 0.9982894 | 1.2787249 | 1.2739726 | 1.1217132 | 1.097673 |
| DNA topoisomerase I | 1.4717432 | 1.2958986 | 1.238216 | 1.2659783 | 1.4154758 | 0.9657008 | 0.8387902 |
| Focal adhesion kinase (pp125FAK) | 1.2089536 | 1.0463614 | 1.2220081 | 1.2794571 | 1.2437392 | 0.9727681 | 1.0716922 |
| NGF-inducible anti-proliferative putative | 1.914832 | 2.2651303 | 3.7566817 | 3.0069327 | 8.955394 | 1.068386 | 0.8254973 |
| secreted protein (PC3) | | | | | | | |
| Phase-1 RCT-82 | 0.9600505 | 0.957578 | 0.8653934 | 1.0532188 | 0.9306153 | 1.0182207 | 1.0333428 |
| Senescence marker protein-30 | 0.6076436 | 0.4440376 | 0.315113 | 0.5811751 | 0.5656847 | 0.8990287 | 0.9056241 |
| Melanoma-associated antigen ME491 | 0.9725763 | 1.3121358 | 0.9265357 | 1.04342 | 1.0614648 | 0.9712471 | 1.2499882 |
| Activating transcription factor 3 | 1.1796467 | 0.940407 | 1.6364841 | 1.3741331 | 1.8530256 | 0.9631472 | 1.2216127 |
| Tryptophan hydroxylase | 1.0389007 | 0.9792817 | 0.6934722 | 0.9015474 | 0.6219958 | 0.9752254 | 0.8964503 |
| Zinc finger protein | 1.6696239 | 2.3295126 | 3.3395178 | 1.7314243 | 4.9114466 | 1.0815872 | 1.1262603 |
| c-myc | 2.0205326 | 1.6513811 | 2.5686285 | 1.8141532 | 2.9426813 | 0.8732964 | 1.0201082 |
| Cholesterol 7-alpha-hydroxylase (P450 | 0.7980877 | 0.6896948 | 0.9728787 | 1.2120649 | 0.4634639 | 0.9355 | 1.1478853 |
| VII) | | | | | | | |
| Ferritin H-chain | 0.7259273 | 0.6381798 | 0.4632858 | 0.572282 | 0.674234 | 1.2500324 | 0.9850146 |
| Phase-1 RCT-197 | 1.079577 | 1.0240341 | 1.1786755 | 1.0349623 | 1.397963 | 1.057768 | 1.07618 |
| Phase-1 RCT-179 | 1.6550967 | 1.8030298 | 1.8449848 | 1.1742322 | 1.8441302 | 1.2266437 | 1.112049 |
| Phase-1 RCT-15 | 1.9458218 | 2.3901346 | 2.08598 | 1.8094372 | 3.7290175 | 1.0159757 | 1.0947173 |
| Argininosuccinate lyase | 2.8587906 | 3.5685694 | 2.4148872 | 1.6894931 | 2.4476388 | 1.0819371 | 1.0244849 |
| Phase-1 RCT-49 | 1.2049912 | 1.4289426 | 1.2147334 | 1.1635863 | 1.431827 | 1.0397378 | 1.1629713 |
| Carbonic anhydrase III | 0.3886747 | 0.8382018 | 0.2439355 | 0.2678276 | 0.2268833 | 1.553966 | 1.3769562 |
| Proliferating cell nuclear antigen gene | 1.403924 | 1.3074068 | 1.2528185 | 1.1876034 | 1.3709092 | 0.935218 | 1.2976757 |
| Integrin beta1 | 1.0191647 | 0.9134213 | 1.4534758 | 0.910799 | 2.275143 | 1.027478 | 0.9618995 |
| Caspase 6 | 1.3798392 | 1.1501068 | 1.0802578 | 1.0356654 | 1.3100941 | 1.1288208 | 1.0118423 |
| Phase-1 RCT-116 | 1.8895009 | 1.944999 | 1.5608617 | 1.7544655 | 1.7949226 | 0.7281658 | 1.1043752 |
| Phase-1 RCT-109 | 1.1161093 | 1.1229287 | 1.2617439 | 1.3744372 | 1.3791358 | 1.127207 | 0.877049 |
| Phase-1 RCT-71 | 1.510902 | 2.0123925 | 1.332907 | 1.4250013 | 1.9319296 | 0.9219413 | 1.1969875 |
| Elongation factor-1 alpha | 1.2228757 | 1.1040053 | 1.0604708 | 0.9927902 | 1.2657409 | 1.1420176 | 0.8734031 |
| Phase-1 RCT-169 | 0.6680861 | 0.8090251 | 0.8306079 | 0.7641866 | 0.9756134 | 0.8641145 | 0.9987432 |
| Pyruvate kinase, muscle | 0.9811322 | 1.0238789 | 1.1094862 | 0.7790501 | 1.0928495 | 1.0584176 | 0.9579143 |
| Nucleosome assembly protein | 0.8832467 | 1.0206528 | 0.6944891 | 0.8681498 | 0.7742369 | 0.553016 | 1.2141889 |
| Phase-1 RCT-127 | 1.6771466 | 1.944247 | 2.8616943 | 1.4297763 | 2.5194345 | 0.9285578 | 0.9643747 |
| Iron-responsive element-binding protein | 0.9758065 | 0.9853613 | 0.5843121 | 0.8603492 | 0.8367039 | 0.9857332 | 1.0796946 |
| Phase-1 RCT-72 | 1.1676978 | 1.3555491 | 1.6707041 | 0.9350559 | 1.4908531 | 0.9505301 | 0.9685665 |
| Phase-1 RCT-144 | 2.5976505 | 2.799812 | 2.1138384 | 1.9525727 | 2.8650112 | 1.0017062 | 1.2311654 |
| Phase-1 RCT-242 | 1.4995015 | 1.9234961 | 4.97803 | 2.601281 | 11.4892845 | 0.9817183 | 1.3860449 |
| 17-beta hydroxysteroid dehydrogenase, | 0.578491 | 0.9195684 | 0.2841888 | 0.353854 | 0.3587458 | 0.4212439 | 1.2421407 |
| type 2 | | | | | | | |
| Phase-1 RCT-70 | 0.870917 | 0.9500216 | 0.8432429 | 1.1055617 | 0.8987861 | 1.032583 | 1.1535485 |
| Ribosomal protein L13A | 1.1916605 | 1.1180195 | 0.9025008 | 1.0176058 | 1.0575814 | 1.0361423 | 0.8240427 |
| Cytochrome P450 2E1 | 0.8217463 | 0.8089083 | 0.7393287 | 0.7798795 | 0.49442 | 1.0716684 | 1.106448 |
| AT-3 | 0.8680952 | 0.9175097 | 0.9317156 | 1.1079845 | 0.9153247 | 1.0514488 | 0.9979278 |
| Phase-1 RCT-270 | 0.7656059 | 0.7836567 | 0.5242405 | 0.660677 | 0.6121206 | 0.989503 | 0.9269268 |
| Phase-1 RCT-123 | 0.9029953 | 0.9943603 | 1.0597963 | 1.0219479 | 0.9845551 | 1.0312549 | 1.1015273 |
| Alpha-2-macroglobulin, sequence 2 | 1.4344763 | 1.5226294 | 1.1021414 | 1.0522622 | 1.2363093 | 1.355085 | 0.9926317 |
| Matrix metalloproteinase-1 | 1.2999136 | 1.0790876 | 0.5837775 | 2.3958344 | 0.6774531 | 1.3127109 | 1.0215379 |
| Beta-tubulin, class I | 3.0734763 | 3.1595273 | 2.6005182 | 2.2041762 | 4.0310283 | 1.0620667 | 1.4178052 |
| Phase-1 RCT-161 | 0.6436908 | 0.9338204 | 0.5731397 | 0.6634454 | 0.4707212 | 1.0779816 | 1.136107 |
| 14-3-3 zeta | 1.5320332 | 1.8057389 | 1.2555195 | 1.2691565 | 1.4776924 | 0.9043232 | 1.1570945 |
| Stem cell factor | 1.0276536 | 0.8536892 | 0.74617 | 1.0709616 | 0.6877525 | 1.152533 | 1.0292016 |
| Macrophage inflammatory protein-2 alpha | 1.6986376 | 1.4569376 | 2.2329133 | 2.3857834 | 1.9290633 | 1.0716646 | 0.9977906 |
| Thymidylate synthase | 0.9309338 | 0.9320583 | 0.900398 | 0.8063534 | 1.0715797 | 0.8162859 | 0.9559851 |
| c-jun | 1.8582604 | 1.8694681 | 2.2903016 | 1.9724668 | 2.6356294 | 0.8907335 | 1.0907642 |
| Superoxide dismutase Mn | 1.0660243 | 0.9990187 | 0.8322479 | 0.7566869 | 1.1502824 | 1.095552 | 1.0217282 |
| Phase-1 RCT-73 | 0.9643959 | 0.9584799 | 0.9231306 | 1.1488204 | 0.8450612 | 1.016989 | 0.9845281 |
| Macrophage inflammatory protein-1 alpha | 0.9249432 | 0.9224536 | 1.2443128 | 0.9552614 | 1.153597 | 0.9454289 | 1.163116 |
| Phase-1 RCT-214 | 1.0540543 | 0.9568391 | 1.0605855 | 0.8932877 | 0.7770945 | 0.8216128 | 1.5341064 |
| NADH-cytochrome b5 reductase | 0.8518867 | 1.1021755 | 0.7962869 | 0.82004 | 0.8416947 | 0.9698979 | 0.9536417 |
| Interleukin-1 beta | 1.0635978 | 1.016666 | 0.8295447 | 1.0873649 | 0.8624718 | 1.1613837 | 1.0029951 |
| NADP-dependent isocitrate | 1.0084207 | 0.7856684 | 0.8014894 | 0.968227 | 0.5438233 | 1.0739686 | 0.9492402 |
| dehydrogenase, cytosolic | | | | | | | |
| Phase-1 RCT-289 | 0.9060668 | 0.8874055 | 0.7109394 | 0.9826971 | 0.6705193 | 1.1287707 | 1.0124767 |
| Glutamine synthetase | 0.992063 | 0.701481 | 1.2314194 | 1.2614123 | 2.1154976 | 1.0293003 | 0.879449 |
| Phase-1 RCT-182 | 1.2192341 | 1.3831582 | 0.8706614 | 0.7764178 | 0.7008016 | 1.0864663 | 0.9236989 |
| Ubiquitin conjugating enzyme (RAD 6 | 1.057868 | 1.1671858 | 1.2701374 | 1.104426 | 1.2518088 | 1.1215034 | 0.7775831 |
| homologue) | | | | | | | |
| Extracellular-signal-regulated kinase 1 | 1.1504676 | 1.1828971 | 0.6734789 | 1.1400086 | 0.6607913 | 1.4305286 | 1.3362181 |
| Emerin | 1.2493126 | 1.3261694 | 1.1256912 | 1.0940326 | 1.2757198 | 0.9623592 | 1.1775948 |
| Phase-1 RCT-78 | 0.9076651 | 1.0470706 | 0.883615 | 0.8757933 | 0.7561464 | 1.0971451 | 1.3947152 |
| Phase-1 RCT-212 | 1.2115074 | 1.2222426 | 1.3288201 | 1.080613 | 1.4374882 | 0.9709001 | 1.0348009 |
| Phase-1 RCT-168 | 1.1739362 | 1.118883 | 1.1318314 | 1.328074 | 0.6458857 | 1.0290341 | 1.0286711 |
| Lysyl hydroxylase | 1.0380856 | 0.9047957 | 0.9308733 | 0.8319472 | 1.1628278 | 1.0190585 | 0.9055197 |
| Phase-1 RCT-55 | 0.846477 | 0.8554151 | 0.7659817 | 0.761888 | 0.823519 | 1.0526472 | 1.0062453 |
| MHC class I antigen RT1.A1(f) alpha- | 1.4968697 | 1.7525383 | 1.3104522 | 1.4503052 | 1.5033327 | 0.9030065 | 1.1345328 |
| chain | | | | | | | |
| Phase-1 RCT-40 | 0.8862413 | 0.9394777 | 0.7965719 | 0.839186 | 0.8490865 | 1.1095096 | 0.987164 |
| Cyclin G | 1.8117791 | 1.9583093 | 1.618793 | 1.471769 | 3.109558 | 0.9288543 | 1.0911125 |
| Peroxisome proliferator activated receptor | 0.9635546 | 0.9376665 | 0.6260203 | 1.022725 | 0.7775657 | 0.7160233 | 0.8227503 |
| gamma | | | | | | | |
| Phase-1 RCT-280 | 0.7444198 | 0.743405 | 0.8394421 | 0.8321009 | 0.7953185 | 1.0345894 | 1.0216712 |
| D-dopachrome tautomerase | 1.6425738 | 1.4984787 | 1.2116263 | 1.4387175 | 1.0553871 | 1.3647795 | 0.8959435 |
| Phase-1 RCT-287 | 1.2379296 | 1.0079588 | 0.9783564 | 1.1718795 | 1.1520829 | 1.0278413 | 0.9880351 |
| Phase-1 RCT-119 | 1.0765576 | 1.4023663 | 0.7980328 | 0.8785276 | 0.6349393 | 1.2747958 | 0.9847147 |
| Thioredoxin-1 (Trx1) | 1.1588246 | 1.0985564 | 1.1384573 | 0.9200273 | 1.2581404 | 1.0934443 | 0.9058038 |
| Phase-1 RCT-225 | 2.5591798 | 1.3646406 | 3.0834296 | 1.3802259 | 3.9944038 | 0.9133483 | 1.3046439 |
| Protein kinase C alpha | 0.8851156 | 0.8764793 | 1.0102537 | 1.0762484 | 0.9842151 | 1.0300496 | 0.930261 |
| Phase-1 RCT-87 | 1.5036851 | 1.556417 | 1.1882132 | 1.2929627 | 1.1018525 | 1.0939481 | 0.9432995 |
| Phase-1 RCT-59 | 1.1816541 | 1.2817224 | 1.4672865 | 1.4905576 | 2.952709 | 1.0825353 | 1.0621262 |
| C4b-binding protein | 0.936726 | 1.0256457 | 0.8250563 | 0.9345675 | 0.6607361 | 0.8661791 | 0.8832536 |
| Phase-1 RCT-204 | 0.9111267 | 0.9899476 | 1.0051489 | 0.9046057 | 0.8936523 | 0.9820424 | 1.080614 |
| Carnitine palmitoyl-CoA transferase | 3.5171864 | 3.0035732 | 1.4658041 | 2.188171 | 1.7567886 | 1.0469853 | 1.0844889 |
| 8-oxoguanine DNA glycosylase | 1.097319 | 1.0081261 | 0.9629749 | 1.0342474 | 0.9680742 | 0.8997161 | 0.9671794 |
| H-rev107 | 0.8720893 | 0.8104222 | 0.9753592 | 0.9597423 | 0.9234449 | 0.9016785 | 1.0579176 |
| Preproalbumin | 1.4227397 | 1.2123443 | 1.3434268 | 1.1290538 | 1.2993124 | 0.8727888 | 0.9897364 |
| Phase-1 RCT-177 | 1.0490078 | 1.030741 | 0.8378254 | 1.0292168 | 0.90443 | 0.9919459 | 0.9113477 |
| Phase-1 RCT-110 | 0.8343279 | 0.9575599 | 0.9467166 | 1.0201768 | 0.9473477 | 0.9374098 | 1.037312 |
| Phase-1 RCT-227 | 0.840459 | 1.0055481 | 0.6432704 | 0.6314482 | 0.6813583 | 1.2865214 | 1.8025203 |
| Neuropeptide Y | 0.9647372 | 0.9666622 | 0.9592631 | 0.9071968 | 0.9903095 | 0.9892066 | 1.0157048 |
| Phase-1 RCT-277 | 0.9475825 | 0.8431503 | 0.644836 | 1.0265075 | 0.7476132 | 0.9288535 | 0.9081618 |
| Phase-1 RCT-229 | 0.8765233 | 0.954728 | 0.9906169 | 0.9763436 | 0.8954263 | 0.9446335 | 1.1001315 |
| Phase-1 RCT-34 | 2.4693944 | 1.7597972 | 1.2272075 | 1.1408653 | 1.0090904 | 1.0986434 | 1.0703478 |
| Choline kinase | 1.7859397 | 1.3411621 | 1.9232041 | 2.068268 | 3.0889084 | 1.1524023 | 0.9392262 |
| Phase-1 RCT-20 | 1.3100215 | 1.2312012 | 1.3019884 | 1.1288543 | 1.2474153 | 1.1002225 | 0.9758862 |
| Phase-1 RCT-248 | 0.8750936 | 0.9966755 | 1.00111 | 1.2209706 | 0.9790316 | 1.0156494 | 0.9645895 |
| | BEN | BEN | BEN | BEN | BAP | BAP | BAP |
| Compound-Dose (2) | 250 | 1000 | 1000 | 1000 | 30 | 30 | 30 |
| Animal Number (3) | 2023 | 2031 | 2032 | 2033 | 2341 | 2342 | 2343 |
| Liver Toxicity Necrosis Classification (4) | no | no | no | no | no | no | no |
| Gene Name (5) | | | | | | | |
| Gadd153 | 0.9683761 | 1.1387544 | 0.9452676 | 0.9515294 | 0.9415396 | 1.008518 | 0.9654388 |
| Interferon related developmental regulator | 0.9095981 | 1.0439614 | 1.1902914 | 0.9706668 | 0.6318749 | 1.0416102 | 1.0051893 |
| IFRD1 (PC4) | | | | | | | |
| ID-1 | 1.0536817 | 1.1008229 | 1.0810982 | 1.0023307 | 1.0071018 | 0.9465805 | 0.9542463 |
| Insulin-like growth factor binding protein 1 | 1.0717798 | 1.0304934 | 1.5515112 | 1.0748788 | 1.3534724 | 0.7378288 | 0.9144203 |
| NIPK | 0.8742918 | 1.3263052 | 1.2025973 | 0.9985361 | 1.4874468 | 1.2075617 | 0.9180057 |
| Phase-1 RCT-50 | 1.1117833 | 1.1301895 | 1.6554614 | 1.0153717 | 0.9361773 | 1.1907914 | 1.014833 |
| Sarcoplasmic reticulum calcium ATPase | 0.9348387 | 1.0755188 | 0.9380012 | 1.0467066 | 0.8337493 | 0.9241457 | 0.999489 |
| Phase-1 RCT-207 | 1.0148903 | 1.0669198 | 1.1017363 | 1.0460603 | 0.9778493 | 0.9879798 | 0.9709057 |
| Acetyl-CoA carboxylase | 0.8281677 | 0.9236103 | 0.8565658 | 0.8831171 | 0.925112 | 1.0469472 | 0.9993674 |
| Cathepsin L, sequence 2 | 1.226452 | 1.292512 | 1.4868503 | 1.280369 | 1.3073148 | 1.0018548 | 1.1605837 |
| Glucokinase | 0.7594352 | 0.9386267 | 0.8855665 | 0.782422 | 0.8930602 | 0.9675494 | 1.3617924 |
| Gadd45 | 1.1544037 | 0.9567532 | 1.4173826 | 1.0051656 | 1.2708099 | 1.2991488 | 1.4683206 |
| Phase-1 RCT-18 | 0.9815613 | 0.9981352 | 0.95618 | 0.9626559 | 0.937878 | 1.0430925 | 0.971598 |
| 3-beta-hydroxysteroid dehydrogenase | 1.0252222 | 1.1046706 | 0.9366363 | 1.0382205 | 0.9281052 | 0.7567052 | 0.8420869 |
| (HSD3B1) | | | | | | | |
| Phase-1 RCT-221 | 0.9006341 | 0.9371884 | 0.8558375 | 0.9508656 | 0.9650573 | 0.8989558 | 0.7638605 |
| Heme oxygenase | 1.0754746 | 1.1956061 | 1.409539 | 1.0226585 | 1.2050859 | 0.9456133 | 0.9079599 |
| Cyclin dependent kinase 4 | 1.0171089 | 1.0312159 | 0.9902147 | 1.0005714 | 1.0685337 | 1.1061859 | 1.0631801 |
| Phase-1 RCT-75 | 1.0002882 | 0.996945 | 1.1225103 | 0.9007581 | 1.1376868 | 1.2508633 | 1.0866421 |
| Phase-1 RCT-65 | 1.0479249 | 1.2152191 | 1.4472709 | 1.0905639 | 1.3336216 | 1.1220204 | 1.019122 |
| Cytochrome P450 1A1 | 1.1124738 | 0.832781 | 1.259691 | 1.0051148 | 1.2191882 | 1.237239 | 1.3378577 |
| Peroxisomal multifunctional enzyme type II | 0.9772015 | 1.014494 | 1.127635 | 1.1291926 | 1.0633434 | 1.109238 | 0.9627082 |
| DNA topoisomerase I | 0.9348186 | 1.1189626 | 0.8519251 | 0.9813798 | 0.9151466 | 0.8651702 | 0.8917974 |
| Focal adhesion kinase (pp125FAK) | 1.0693499 | 1.0599475 | 1.1384406 | 0.9318082 | 0.9915662 | 1.0257202 | 0.9959123 |
| NGF-inducible anti-proliferative putative | 0.7710759 | 0.9473479 | 0.885401 | 0.8494008 | 0.9479582 | 1.0350783 | 0.7986226 |
| secreted protein (PC3) | | | | | | | |
| Phase-1 RCT-82 | 0.9886292 | 0.992726 | 1.0045571 | 1.053785 | 1.0144799 | 1.0352397 | 0.9392172 |
| Senescence marker protein-30 | 0.7703715 | 0.9048498 | 0.4903531 | 0.7862633 | 1.0722827 | 0.7759587 | 1.1280499 |
| Melanoma-associated antigen ME491 | 1.1408322 | 1.2807392 | 1.054246 | 0.8284736 | 0.9177795 | 1.0254962 | 0.9915676 |
| Activating transcription factor 3 | 1.0438858 | 0.9382019 | 1.0266302 | 1.140475 | 1.1268996 | 0.9459148 | 0.995845 |
| Tryptophan hydroxylase | 0.9261074 | 0.8884928 | 1.1281359 | 0.9335435 | 1.1253415 | 1.068326 | 1.1382437 |
| Zinc finger protein | 1.0347207 | 0.943564 | 1.0613304 | 0.945529 | 0.9707221 | 0.902717 | 0.9417827 |
| c-myc | 1.0291198 | 0.9782018 | 1.1184841 | 1.1237218 | 0.9432874 | 0.956439 | 0.9128098 |
| Cholesterol 7-alpha-hydroxylase (P450 | 0.879029 | 0.6366271 | 0.5679188 | 0.7674264 | 0.9602838 | 1.0467876 | 0.887899 |
| VII) | | | | | | | |
| Ferritin H-chain | 0.9850194 | 0.9726592 | 0.9390332 | 1.0208918 | 1.1963959 | 0.9338194 | 1.0811028 |
| Phase-1 RCT-197 | 1.031227 | 1.0368139 | 1.105129 | 1.0986933 | 0.9839378 | 1.064774 | 0.9465814 |
| Phase-1 RCT-179 | 1.034931 | 1.2662401 | 1.0775819 | 1.0372267 | 1.042199 | 0.9479235 | 1.0046651 |
| Phase-1 RCT-15 | 1.1597381 | 1.271488 | 1.3232657 | 1.1758727 | 0.8864738 | 1.0550056 | 0.9621097 |
| Argininosuccinate lyase | 1.2148846 | 1.2928277 | 1.3599861 | 1.0105871 | 1.2600881 | 1.0079525 | 1.4069893 |
| Phase-1 RCT-49 | 1.0358545 | 1.1198446 | 1.018911 | 0.9605095 | 0.9930345 | 1.044677 | 1.0576253 |
| Carbonic anhydrase III | 1.1699673 | 0.9092153 | 0.290493 | 0.8785826 | 0.744348 | 0.518957 | 0.5612943 |
| Proliferating cell nuclear antigen gene | 0.9995413 | 0.9725171 | 1.0424138 | 1.0504996 | 0.9029927 | 1.0552375 | 0.9963119 |
| Integrin beta1 | 1.1099677 | 1.1594422 | 1.2221786 | 1.1600702 | 1.153276 | 0.9791925 | 0.9688249 |
| Caspase 6 | 1.0112442 | 1.0864149 | 1.0586573 | 1.047405 | 1.092894 | 0.9838504 | 1.0308443 |
| Phase-1 RCT-116 | 1.110397 | 1.214551 | 1.1358361 | 1.2505432 | 0.9276549 | 1.0343716 | 1.0981034 |
| Phase-1 RCT-109 | 0.9773294 | 1.0756593 | 0.9822745 | 0.9599237 | 0.9894444 | 1.0157758 | 1.0539043 |
| Phase-1 RCT-71 | 0.8056921 | 0.9783335 | 0.8728668 | 1.0502499 | 1.2519714 | 1.199545 | 1.1203063 |
| Elongation factor-1 alpha | 0.9354629 | 1.0330127 | 0.9176871 | 1.0042218 | 1.2135237 | 1.0966767 | 1.2003436 |
| Phase-1 RCT-169 | 1.1302775 | 1.0221606 | 1.1331176 | 1.0322253 | 0.7711418 | 1.0613542 | 0.9930456 |
| Pyruvate kinase, muscle | 1.1109506 | 1.1110457 | 1.2025235 | 1.0612884 | 1.0512054 | 1.084391 | 1.024586 |
| Nucleosome assembly protein | 0.5948792 | 0.8922827 | 0.3964799 | 1.1437826 | 0.7854957 | 1.1046975 | 1.1333194 |
| Phase-1 RCT-127 | 0.9061555 | 1.0382477 | 1.1604956 | 0.9046334 | 0.9197086 | 1.0473063 | 1.071945 |
| Iron-responsive element-binding protein | 0.8374215 | 0.9013534 | 0.9034723 | 0.9853731 | 1.1328592 | 1.0990165 | 1.0408896 |
| Phase-1 RCT-72 | 1.01911 | 1.1106812 | 0.9888034 | 1.0734684 | 0.8700177 | 1.0917567 | 1.0657859 |
| Phase-1 RCT-144 | 1.1519616 | 1.1261474 | 1.0943449 | 1.0185765 | 0.857712 | 1.0796282 | 1.001341 |
| Phase-1 RCT-242 | 1.1493311 | 1.1277505 | 1.3467069 | 0.9802957 | 1.0186136 | 1.0789002 | 0.9350919 |
| 17-beta hydroxysteroid dehydrogenase, | 0.4703964 | 0.8372051 | 0.1537785 | 1.2012304 | 0.4908962 | 1.236198 | 1.6672584 |
| type 2 | | | | | | | |
| Phase-1 RCT-70 | 1.0559789 | 1.0796541 | 1.2262121 | 1.1394027 | 1.0419157 | 1.0635666 | 0.8998135 |
| Ribosomal protein L13A | 1.0075092 | 1.0514439 | 0.9552864 | 0.9261219 | 1.0221624 | 1.0358661 | 1.1063783 |
| Cytochrome P450 2E1 | 0.8185985 | 0.6378579 | 0.8383358 | 1.2942786 | 1.6571988 | 1.3947438 | 1.5483325 |
| AT-3 | 1.0514518 | 1.0863788 | 1.1334628 | 1.0761588 | 0.8829327 | 0.9971089 | 0.841878 |
| Phase-1 RCT-270 | 0.6764457 | 0.8720043 | 0.5426679 | 0.8362474 | 0.9007188 | 0.8223022 | 0.9738163 |
| Phase-1 RCT-123 | 1.0274695 | 1.0060921 | 1.1269206 | 1.0010532 | 0.9301156 | 0.9754632 | 0.9480589 |
| Alpha-2-macroglobulin, sequence 2 | 1.0431353 | 1.1370115 | 1.1299165 | 1.1421741 | 0.9678952 | 1.1149094 | 1.0400656 |
| Matrix metalloproteinase-1 | 1.064139 | 1.1204358 | 1.0842026 | 1.1142659 | 1.1589211 | 0.9670295 | 1.0406342 |
| Beta-tubulin, class I | 1.0993313 | 1.0786425 | 0.8089006 | 0.9210888 | 0.8369234 | 0.994945 | 0.958302 |
| Phase-1 RCT-161 | 1.0563987 | 1.0216721 | 1.1776572 | 0.9661908 | 0.9353804 | 1.0573982 | 0.9455785 |
| 14-3-3 zeta | 1.1075889 | 1.0827193 | 1.0327682 | 0.9628457 | 1.0311359 | 1.038877 | 0.98365 |
| Stem cell factor | 0.8448905 | 1.0418656 | 0.715937 | 0.8952109 | 0.9195058 | 0.9170446 | 1.0569612 |
| Macrophage inflammatory protein-2 alpha | 1.362949 | 1.2373819 | 2.1291444 | 1.2273024 | 1.1747035 | 1.1052834 | 1.0462251 |
| Thymidylate synthase | 0.8534631 | 0.8668214 | 0.7610875 | 1.0161531 | 1.0199044 | 1.101687 | 0.9376044 |
| c-jun | 1.2735114 | 1.0538281 | 1.3020523 | 1.1935297 | 0.9770911 | 1.1124682 | 1.0067432 |
| Superoxide dismutase Mn | 0.9754357 | 0.9188979 | 0.9981703 | 1.0945923 | 1.1759368 | 1.210189 | 1.0883915 |
| Phase-1 RCT-73 | 0.912441 | 1.0235337 | 1.0541768 | 0.9722236 | 1.1255492 | 1.1050091 | 1.0694886 |
| Macrophage inflammatory protein-1 alpha | 1.317512 | 2.12987 | 1.3809446 | 1.0288526 | 1.0061669 | 0.9673199 | 1.0828346 |
| Phase-1 RCT-214 | 0.8929969 | 0.7957775 | 0.5479866 | 0.8374233 | 0.8316043 | 0.8409492 | 1.0976923 |
| NADH-cytochrome b5 reductase | 0.7670028 | 0.8907043 | 0.6850178 | 0.9748021 | 1.318038 | 1.0787482 | 1.0765324 |
| Interleukin-1 beta | 1.0518932 | 1.0426127 | 1.1069993 | 1.010734 | 1.059884 | 1.0415621 | 1.0568024 |
| NADP-dependent isocitrate | 0.7997742 | 0.9630634 | 0.6466874 | 0.9313159 | 1.0383492 | 0.9655658 | 1.1109564 |
| dehydrogenase, cytosolic | | | | | | | |
| Phase-1 RCT-289 | 0.8605115 | 0.8655248 | 0.7888005 | 0.9560064 | 1.1264015 | 1.0624944 | 1.0771321 |
| Glutamine synthetase | 0.985204 | 0.9218724 | 0.6655083 | 1.0000782 | 1.0942863 | 0.7622418 | 0.8726466 |
| Phase-1 RCT-182 | 0.744724 | 0.7713571 | 0.6825076 | 0.9038299 | 1.1004655 | 0.9374647 | 1 |
| Ubiquitin conjugating enzyme (RAD 6 | 0.853883 | 0.9874579 | 0.8972662 | 0.8485302 | 0.9909887 | 0.9505234 | 1.0009576 |
| homologue) | | | | | | | |
| Extracellular-signal-regulated kinase 1 | 1.1243943 | 0.9843239 | 0.5190032 | 0.9368947 | 0.81425 | 0.6573305 | 0.7010246 |
| Emerin | 0.9395209 | 0.9881254 | 0.9477368 | 1.1788602 | 0.9304916 | 0.969379 | 0.9959185 |
| Phase-1 RCT-78 | 0.9447072 | 1.0777075 | 1.0196669 | 1.0980341 | 0.9636014 | 0.9806593 | 0.9868971 |
| Phase-1 RCT-212 | 1.0131453 | 1.0594159 | 0.9069422 | 1.0486423 | 1.207818 | 1.146252 | 1.1241823 |
| Phase-1 RCT-168 | 0.6993837 | 0.8576606 | 0.5339859 | 0.8534265 | 0.8789651 | 0.8911815 | 1.1608813 |
| Lysyl hydroxylase | 0.9649132 | 0.9284635 | 0.8177961 | 0.9473484 | 1.0548344 | 0.9080069 | 0.8974394 |
| Phase-1 RCT-55 | 1.2045653 | 1.5779655 | 0.8353053 | 1.115457 | 0.9038845 | 1.1947882 | 1.0233783 |
| MHC class I antigen RT1.A1(f) alpha- | 1.1553309 | 1.157107 | 1.2515545 | 1.1741903 | 1.096581 | 1.1168116 | 1.2572243 |
| chain | | | | | | | |
| Phase-1 RCT-40 | 0.9765561 | 0.9976908 | 0.8128642 | 1.0579932 | 0.8700024 | 0.8271677 | 0.8514841 |
| Cyclin G | 1.223063 | 1.0394315 | 1.2198827 | 1.1107566 | 1.0720488 | 1.0913224 | 1.1404341 |
| Peroxisome proliferator activated receptor | 0.6163117 | 0.7980248 | 0.6249651 | 1.0803914 | 1.0839412 | 1.2062597 | 0.9764356 |
| gamma | | | | | | | |
| Phase-1 RCT-280 | 1.1429276 | 0.8652598 | 1.0053462 | 1.0177286 | 1.1412182 | 0.9327691 | 1.1380948 |
| D-dopachrome tautomerase | 0.9486489 | 0.9606651 | 0.6998898 | 0.8762071 | 1.0316594 | 0.9653901 | 1.0396582 |
| Phase-1 RCT-287 | 1.0061791 | 1.146569 | 1.2208104 | 1.1244864 | 1.0108587 | 0.94835 | 0.8705846 |
| Phase-1 RCT-119 | 0.9491282 | 0.9087423 | 1.1121122 | 1.0502428 | 0.9757909 | 0.9145449 | 0.8978524 |
| Thioredoxin-1 (Trx1) | 0.8631975 | 0.8911324 | 0.8404467 | 1.0469868 | 0.9577307 | 1.0829806 | 1.2038869 |
| Phase-1 RCT-225 | 1.3535538 | 1.3432908 | 0.8849381 | 1.2128373 | 3.1801095 | 1.6650091 | 1.3840051 |
| Protein kinase C alpha | 1.1094604 | 0.9130698 | 0.9102957 | 0.9757217 | 0.9779568 | 1.1319137 | 0.9893307 |
| Phase-1 RCT-87 | 0.9212182 | 0.9735436 | 0.9306746 | 1.0143461 | 1.0650865 | 1.0825604 | 1.1497867 |
| Phase-1 RCT-59 | 1.051865 | 0.9647018 | 1.0979718 | 1.0629542 | 0.8823918 | 0.8257269 | 0.8758298 |
| C4b-binding protein | 0.8866565 | 1.0791072 | 1.0733774 | 0.8757234 | 0.7482706 | 0.8761343 | 0.8210863 |
| Phase-1 RCT-204 | 0.9785676 | 1.0934997 | 1.1865572 | 1.0431378 | 1.0596333 | 1.0131397 | 1.0274467 |
| Carnitine palmitoyl-CoA transferase | 1.01117 | 0.9100943 | 1.1136179 | 1.035553 | 1.1611589 | 1.0578135 | 1.0761838 |
| 8-oxoguanine DNA glycosylase | 1.075637 | 1.060725 | 1.0661694 | 0.9283683 | 1.0521836 | 1.0913515 | 0.998203 |
| H-rev107 | 0.9471097 | 0.9344137 | 0.9308853 | 0.9533712 | 1.0005459 | 0.88879 | 0.9975345 |
| Preproalbumin | 0.9224665 | 1.0253764 | 0.6973596 | 1.0031093 | 1.1283779 | 0.8631486 | 0.9748936 |
| Phase-1 RCT-177 | 0.8947864 | 1.0349624 | 1.0889244 | 0.9657174 | 1.2596984 | 1.160194 | 1.2743162 |
| Phase-1 RCT-110 | 1.0219785 | 0.9757128 | 1.0050547 | 0.9552113 | 0.9231288 | 1.0853168 | 0.9039382 |
| Phase-1 RCT-227 | 1.0132759 | 1.2296067 | 1.1625408 | 1.240913 | 1.1962849 | 0.9940471 | 1.0150079 |
| Neuropeptide Y | 1.010097 | 1.0439422 | 1 | 1.0599746 | 0.9062545 | 0.985494 | 1.0805749 |
| Phase-1 RCT-277 | 0.8276205 | 1.0010785 | 0.7394584 | 1.143283 | 1.0313994 | 0.7643012 | 1.065428 |
| Phase-1 RCT-229 | 1.0371146 | 1.0145515 | 1.1772475 | 1.056379 | 0.928556 | 1.08085 | 0.9575524 |
| Phase-1 RCT-34 | 1.1493608 | 1.4026551 | 0.8901727 | 1.1991684 | 1.0469079 | 1.43809 | 1.0635649 |
| Choline kinase | 0.8888829 | 1.0516448 | 0.9098578 | 0.9481391 | 1.1818445 | 0.9343685 | 1.2065281 |
| Phase-1 RCT-20 | 1.0342422 | 1.0430638 | 1.0476933 | 1.0723549 | 0.9686995 | 1.013308 | 1.040619 |
| Phase-1 RCT-248. | 0.9830865 | 0.994502 | 0.9885486 | 0.9300904 | 0.9814531 | 1.0541953 | 1.0052253 |
| | BRB | BRB | BRB | BRB | BRB | BRB | BUS |
| Compound-Dose (2) | 200 | 200 | 200 | 800 | 800 | 800 | 14 |
| Animal Number (3) | 2321 | 2322 | 2323 | 2331 | 2332 | 2333 | 1741 |
| Liver Toxicity Necrosis Classification (4) | yes | yes | yes | yes | yes | yes | no |
| Gene Name (5) | | | | | | | |
| Gadd153 | 1.0996547 | 0.8341616 | 0.8285781 | 1.0651655 | 1.0922648 | 0.7479399 | 1.0389175 |
| Interferon related developmental regulator | 1.5784473 | 0.8766664 | 0.9521281 | 1.1416252 | 1.0262192 | 1.0505267 | 1.1008086 |
| IFRD1 (PC4) | | | | | | | |
| ID-1 | 0.9345724 | 0.9537039 | 1.2841669 | 0.9230764 | 0.9700647 | 0.8780122 | 0.831251 |
| Insulin-like growth factor binding protein 1 | 10.201994 | 1.949942 | 1.915724 | 1.3218093 | 2.7499552 | 2.3119965 | 2.2355814 |
| NIPK | 2.3261423 | 1.6631947 | 2.8327847 | 1.1164727 | 1.7091045 | 1.1146551 | 0.9578965 |
| Phase-1 RCT-50 | 0.9965264 | 0.7723147 | 0.837535 | 0.7891324 | 0.8770608 | 0.8290903 | 0.9361803 |
| Sarcoplasmic reticulum calcium ATPase | 0.9142339 | 0.7398658 | 0.6591609 | 0.7543346 | 0.8128965 | 0.7316823 | 0.9802074 |
| Phase-1 RCT-207 | 1.8528723 | 1.177977 | 1.2360649 | 1.2242993 | 1.4681867 | 1.4105413 | 1.2242144 |
| Acetyl-CoA carboxylase | 1.1107398 | 0.8904163 | 0.8244692 | 0.8188807 | 1.0256019 | 0.9007779 | 0.9697701 |
| Cathepsin L, sequence 2 | 2.3831332 | 1.9324304 | 1.8287247 | 1.7944392 | 1.7731409 | 2.0964768 | 1.1590776 |
| Glucokinase | 0.4516861 | 0.6246617 | 0.5576909 | 0.7269739 | 0.3971601 | 0.4234179 | 1.3270768 |
| Gadd45 | 1.966831 | 1.8286704 | 1.9349273 | 1.1067592 | 2.098856 | 1.6932851 | 0.7213792 |
| Phase-1 RCT-18 | 0.6483723 | 0.7278831 | 0.695882 | 0.7413752 | 0.8239311 | 0.7648718 | 0.8997127 |
| 3-beta-hydroxysteroid dehydrogenase | 0.8558663 | 0.7807798 | 1.020451 | 0.8422508 | 0.584767 | 0.6884784 | 1.1303773 |
| (HSD3B1) | | | | | | | |
| Phase-1 RCT-221 | 1.3382692 | 1.2492766 | 1.0550431 | 1.3420081 | 1.2916902 | 1.40588 | 1.0384121 |
| Heme oxygenase | 2.4110518 | 11.530977 | 10.655806 | 2.40087 | 2.5309384 | 5.924109 | 1.1563706 |
| Cyclin dependent kinase 4 | 1.2767262 | 0.8975322 | 0.7757275 | 0.7427683 | 0.9612296 | 0.8543618 | 1.1194769 |
| Phase-1 RCT-75 | 1.9877325 | 1.1161382 | 1.2327073 | 1.2577907 | 1.2701322 | 1.319002 | 0.9265889 |
| Phase-1 RCT-65 | 1.0819929 | 1.4664961 | 1.2941734 | 0.9051058 | 1.1975101 | 1.0102608 | 1.3893318 |
| Cytochrome P450 1A1 | 0.5754142 | 0.6362601 | 0.6176596 | 0.5952776 | 0.7038136 | 0.6673306 | 0.7840971 |
| Peroxisomal multifunctional enzyme type II | 1.9945956 | 1.9337581 | 1.8582721 | 1.8599674 | 1.7225305 | 2.4660795 | 1.3210993 |
| DNA topoisomerase I | 1.0616039 | 0.8123445 | 0.7682843 | 0.8117516 | 0.8388233 | 0.9501295 | 1.1830039 |
| Focal adhesion kinase (pp125FAK) | 0.8537523 | 0.7899053 | 0.7627546 | 0.7147601 | 0.7548839 | 0.7436317 | 0.9096544 |
| NGF-inducible anti-proliferative putative | 1.7038462 | 1.0966772 | 0.9887268 | 1.1338862 | 1.2199774 | 1.1979269 | 1.08044 |
| secreted protein (PC3) | | | | | | | |
| Phase-1 RCT-82 | 0.5930594 | 0.7070903 | 0.6693354 | 0.6598871 | 0.7869827 | 0.7364691 | 0.9209407 |
| Senescence marker protein-30 | 0.690469 | 0.6343933 | 0.6644554 | 0.5913708 | 0.3295118 | 0.4955376 | 1.0416205 |
| Melanoma-associated antigen ME491 | 0.6928654 | 1.1768833 | 1.1099266 | 1.0548519 | 1.1472411 | 1.088565 | 1.4265038 |
| Activating transcription factor 3 | 0.8559317 | 0.8377538 | 0.8614703 | 0.9157254 | 0.8788609 | 0.8705517 | 1.113728 |
| Tryptophan hydroxylase | 1.0093024 | 0.9064729 | 0.8556363 | 0.9505585 | 0.9206746 | 0.8895429 | 1.2847296 |
| Zinc finger protein | 1.8229138 | 1.0273241 | 0.9795331 | 0.8991521 | 0.8560659 | 0.8593511 | 11133416 |
| c-myc | 2.1182606 | 1.8631449 | 1.5257095 | 0.9941691 | 1.561438 | 1.3135252 | 0.8481485 |
| Cholesterol 7-alpha-hydroxylase (P450 | 0.6171367 | 0.6468103 | 0.6329349 | 0.6096364 | 0.8315015 | 0.6771509 | 2.4100943 |
| VII) | | | | | | | |
| Ferritin H-chain | 1.3253969 | 1.675352 | 1.5554856 | 1.2707413 | 1.089117 | 1.3237996 | 0.889322 |
| Phase-1 RCT-197 | 0.679455 | 0.9596407 | 0.8594266 | 0.781686 | 0.9814485 | 0.8449809 | 1.4460373 |
| Phase-1 RCT-179 | 2.6465857 | 1.4135592 | 1.8323255 | 1.7396343 | 1.4186548 | 1.9355127 | 1.0225435 |
| Phase-1 RCT-15 | 3.1092436 | 1.6220695 | 1.5565863 | 1.415154 | 1.8251929 | 1.6371068 | 1.4660423 |
| Argininosuccinate lyase | 4.6747584 | 2.257461 | 2.1613784 | 2.7580504 | 3.6661165 | 3.846397 | 2.3255715 |
| Phase-1 RCT-49 | 1.3364066 | 0.9298994 | 0.9409059 | 0.9159778 | 1.0552372 | 0.969761 | 1.179395 |
| Carbonic anhydrase III | 0.298023 | 0.1577575 | 0.360401 | 0.588479 | 0.2293877 | 0.3440649 | 0.8058969 |
| Proliferating cell nuclear antigen gene | 0.9455317 | 0.8653094 | 0.7725962 | 0.7000924 | 0.9969806 | 0.7797875 | 0.98162 |
| Integrin beta1 | 1.3656108 | 1.033444 | 0.8836152 | 0.9321238 | 1.3583691 | 1.2590222 | 1.1145693 |
| Caspase 6 | 1.0704948 | 1.151978 | 1.0238767 | 1.1292125 | 1.0856743 | 0.9369038 | 1.0817986 |
| Phase-1 RCT-116 | 1.7074419 | 1.7303934 | 1.72958 | 1.4581916 | 1.616117 | 1.5473114 | 1.7672663 |
| Phase-1 RCT-109 | 1.5408211 | 1.2541593 | 1.275519 | 1.469945 | 1.3869876 | 1.5253252 | 1.1447014 |
| Phase-1 RCT-71 | 2.187869 | 1.2599368 | 1.2598258 | 1.3694009 | 1.6476622 | 1.4726019 | 1.0611848 |
| Elongation factor-1 alpha | 1.8239547 | 1.4228185 | 1.4122667 | 1.6975929 | 1.3356714 | 1.7175736 | 1.2752038 |
| Phase-1 RCT-169 | 0.7517752 | 0.8335239 | 0.7773873 | 0.7521226 | 0.8086913 | 0.6917185 | 0.7179146 |
| Pyruvate kinase, muscle | 0.912553 | 0.9601898 | 0.3389 | 0.9522002 | 1.0912663 | 0.920188 | 0.8775584 |
| Nucleosome assembly protein | 0.6807067 | 0.8218312 | 0.8054949 | 0.7931277 | 0.6928482 | 0.6964256 | 1.273891 |
| Phase-1 RCT-127 | 1.4608772 | 0.9363015 | 0.9695395 | 0.7510293 | 0.8722803 | 0.8111666 | 0.938377 |
| Iron-responsive element-binding protein | 1.2842793 | 1.8240596 | 1.5485758 | 1.4097923 | 1.6682738 | 1.6793947 | 0.9736915 |
| Phase-1 RCT-72 | 0.6654249 | 0.7733882 | 0.7534655 | 0.7031924 | 0.7082347 | 0.6669027 | 0.8364289 |
| Phase-1 RCT-144 | 1.9757348 | 1.1850276 | 0.8853574 | 1.1040978 | 1.3341564 | 1.5797241 | 0.9400913 |
| Phase-1 RCT-242 | 0.985811 | 0.791901 | 0.7287505 | 0.7526717 | 0.9785534 | 0.8382109 | 0.9641146 |
| 17-beta hydroxysteroid dehydrogenase, | 0.2159493 | 0.5894142 | 0.481402 | 0.7140688 | 0.3313084 | 0.3451148 | 0.6145118 |
| type 2 | | | | | | | |
| Phase-1 RCT-70 | 0.786287 | 1.0665656 | 0.8820705 | 1.0351055 | 1.324151 | 1.1347275 | 0.9550574 |
| Ribosomal protein L13A | 1.52151 | 1.1396385 | 1.3989338 | 1.4078625 | 1.2625325 | 1.3627646 | 1.0361149 |
| Cytochrome P450 2E1 | 0.7390931 | 1.1130149 | 1.0533737 | 0.8720703 | 0.8722912 | 0.7795487 | 1.0779967 |
| AT-3 | 0.6174436 | 0.7008765 | 0.6499755 | 0.5786419 | 0.7182002 | 0.6058408 | 0.7546564 |
| Phase-1 RCT-270 | 0.9834291 | 1.1652379 | 1.1633575 | 1.0940489 | 1.1839353 | 0.9952139 | 1.1021551 |
| Phase-1 RCT-123 | 0.7578414 | 0.7655469 | 0.7853172 | 0.7036611 | 0.8002546 | 0.7741383 | 0.9332281 |
| Alpha-2-macroglobulin, sequence 2 | 3.9010272 | 1.3892564 | 1.4140908 | 1.7044066 | 1.6570169 | 1.6258769 | 1.3811414 |
| Matrix metalloproteinase-1 | 1.3347169 | 1.0177312 | 0.9206107 | 0.9669101 | 1.0346707 | 1.0067065 | 1.0934616 |
| Beta-tubulin, class I | 1.1412523 | 1.3404566 | 1.4790536 | 0.868248 | 1.006244 | 1.0859166 | 1.3110378 |
| Phase-1 RCT-161 | 0.492212 | 0.5182843 | 0.590164 | 0.6719332 | 0.6313142 | 0.6034442 | 0.919013 |
| 14-3-3 zeta | 1.3050028 | 1.0551736 | 1.2210464 | 0.9777864 | 1.2110108 | 1.3846525 | 1.1407305 |
| Stem cell factor | 0.7942565 | 0.9180558 | 1.3932098 | 0.9937483 | 0.7548839 | 0.6593459 | 1.0723841 |
| Macrophage inflammatory protein-2 alpha | 2.151789 | 0.8808751 | 0.9215701 | 1.1774691 | 1.1530933 | 0.8278021 | 0.9936696 |
| Thymidylate synthase | 0.9302471 | 0.8669094 | 0.7892742 | 0.6761102 | 0.8378952 | 0.7728633 | 0.898893 |
| c-jun | 1.3871436 | 1.5995406 | 1.38898 | 1.3291215 | 2.0794258 | 1.0179287 | 1.339199 |
| Superoxide dismutase Mn | 1.6784772 | 1.2597113 | 1.2338262 | 1.0899724 | 1.3784454 | 1.1204505 | 1.3652031 |
| Phase-1 RCT-73 | 0.9253287 | 0.9835848 | 1 | 1.0010222 | 1.0421593 | 1.0109471 | 0.9288265 |
| Macrophage inflammatory protein-1 alpha | 0.7806529 | 0.8362705 | 0.8623782 | 0.9778156 | 1.0300423 | 0.7422691 | 1.0655125 |
| Phase-1 RCT-214 | 0.7905642 | 0.8760967 | 0.8647711 | 0.7188382 | 0.7223442 | 0.8528692 | 0.9816945 |
| NADH-cytochrome b5 reductase | 1.1621958 | 1.230008 | 1.3096722 | 0.995016 | 1.0374655 | 1.1474973 | 1.1826204 |
| Interleukin-1 beta | 1.0956602 | 0.9960011 | 0.8980877 | 0.9280265 | 0.9181809 | 0.9555702 | 0.9161333 |
| NADP-dependent isocitrate | 0.9316405 | 1.5300967 | 1.5082638 | 1.1711409 | 0.8654622 | 1.2047809 | 0.8840693 |
| dehydrogenase, cytosolic | | | | | | | |
| Phase-1 RCT-289 | 1.1012759 | 1.0945505 | 1.1192117 | 1.1585194 | 1.0773658 | 1.1815758 | 1.0667849 |
| Glutamine synthetase | 1.0386727 | 0.870734 | 0.9528385 | 1.1147155 | 0.9374233 | 0.9192255 | 1.3063034 |
| Phase-1 RCT-182 | 1.2785391 | 1.2880173 | 1.2910417 | 1.0826452 | 1.156955 | 1.4102608 | 1.0597143 |
| Ubiquitin conjugating enzyme (RAD 6 | 1.6548876 | 0.9269145 | 1.0741147 | 1.226962 | 1.094108 | 1.3416165 | 1.0187544 |
| homologue) | | | | | | | |
| Extracellular-signal-regulated kinase 1 | 0.7390304 | 0.901338 | 0.8014678 | 0.8443065 | 0.8804385 | 0.6572261 | 1.1021793 |
| Emerin | 0.898049 | 0.8360968 | 0.8904551 | 0.8565506 | 0.9428818 | 0.945158 | 1.0161226 |
| Phase-1 RCT-78 | 0.9503455 | 0.8928123 | 0.8291077 | 0.9522306 | 0.9427583 | 0.9550169 | 0.9982413 |
| Phase-1 RCT-212 | 0.9570021 | 1.0007759 | 0.9769918 | 0.9145648 | 0.9466004 | 0.9394206 | 0.9440548 |
| Phase-1 RCT-168 | 0.6199149 | 0.8662243 | 0.7177544 | 0.8327423 | 0.7648168 | 0.9181477 | 0.9421744 |
| Lysyl hydroxylase | 1.2107565 | 1.1008449 | 0.956959 | 0.9010016 | 0.9487892 | 1.0381304 | 1.1233381 |
| Phase-1 RCT-55 | 0.8097586 | 0.8096507 | 0.7681305 | 0.8103089 | 0.7967369 | 0.804613 | 0.6326391 |
| MHC class I antigen RT1.A1(f) alpha- | 0.8896583 | 1.4078712 | 1.3185018 | 1.0934252 | 1.3201215 | 1.0681044 | 1.4629936 |
| chain | | | | | | | |
| Phase-1 RCT-40 | 0.9448729 | 1 | 1.0164052 | 0.9902728 | 0.8077492 | 0.9760939 | 1.1832219 |
| Cyclin G | 1.0329841 | 1.0828401 | 0.8815802 | 0.6840803 | 1.3927739 | 0.7824177 | 1.4571083 |
| Peroxisome proliferator activated receptor | 0.6912981 | 0.8500497 | 0.7911545 | 0.8573994 | 0.99068411 | 0.699567 | 0.8038329 |
| gamma | | | | | | | |
| Phase-1 RCT-280 | 0.9534001 | 0.8615733 | 0.8786874 | 0.8189229 | 0.8114135 | 0.7610647 | 0.9692596 |
| D-dopachrome tautomerase | 1.384319 | 1.7912011 | 1.4327333 | 1.7303877 | 1.282589 | 1.9580717 | 1.0338011 |
| Phase-1 RCT-287 | 1.6417862 | 1.2579132 | 1.1991845 | 1.2757661 | 1.4070396 | 1.3527446 | 1.1625185 |
| Phase-1 RCT-119 | 1.5167423 | 0.9695211 | 0.9043258 | 1.0774713 | 0.9986262 | 0.9328595 | 0.9447048 |
| Thioredoxin-1 (Trx1) | 1.2181594 | 1.2365285 | 1.1742849 | 1.1787622 | 1.0091839 | 1.0936756 | 1.0505008 |
| Phase-1 RCT-225 | 0.601233 | 1.9104548 | 1.4262059 | 0.4768281 | 0.3967885 | 0.6774557 | 1.3270012 |
| Protein kinase C alpha | 0.6601613 | 0.7563042 | 0.7039489 | 0.6994292 | 0.81805 | 0.7225568 | 0.8838576 |
| Phase-1 RCT-87 | 1.2923447 | 1.2091386 | 1.1269919 | 1.0017382 | 1.07127 | 1.213759 | 1.1031731 |
| Phase-1 RCT-59 | 1.9374493 | 1.0248809 | 1.0004367 | 1.2642226 | 1.5928037 | 1.4063356 | 1.3874239 |
| C4b-binding protein | 1.7470182 | 0.7209359 | 0.8697181 | 0.9409187 | 0.5876558 | 1.0117071 | 0.9160246 |
| Phase-1 RCT-204 | 0.9620059 | 0.9292848 | 0.9147794 | 0.8126935 | 0.9375712 | 0.9099326 | 1.0200256 |
| Carnitine palmitoyl-CoA transferase | 1.6289271 | 1.7968493 | 1.5022124 | 2.0522587 | 2.8468359 | 2.903596 | 1.6076658 |
| 8-oxoguanine DNA glycosylase | 0.6756716 | 0.7544016 | 0.6896413 | 0.6357594 | 0.8341391 | 0.6889709 | 0.8889995 |
| H-rev107 | 0.7940808 | 0.7864425 | 0.8106061 | 0.7153174 | 0.7776324 | 0.7940541 | 0.9012175 |
| Preproalbumin | 2.1478064 | 1.8562509 | 2.0377367 | 1.9479977 | 1.498921 | 1.7894061 | 1.1339426 |
| Phase-1 RCT-177 | 1.7963465 | 1.42277 | 1.5658277 | 1.3966238 | 1.4226948 | 1.6020895 | 1.1060225 |
| Phase-1 RCT-110 | 0.5724095 | 0.674163 | 0.6833774 | 0.5994517 | 0.7201641 | 0.6088674 | 0.8139944 |
| Phase-1 RCT-227 | 2.2253728 | 1.7776005 | 1.6296176 | 1.6157794 | 1.6271541 | 1.6645541 | 1.3053432 |
| Neuropeptide Y | 0.6518005 | 0.7220946 | 0.7173794 | 0.6652931 | 0.7889586 | 0.7619904 | 0.8015558 |
| Phase-1 RCT-277 | 0.9123513 | 0.9365035 | 0.8474994 | 0.8244242 | 0.8098863 | 0.8648926 | 0.806389 |
| Phase-1 RCT-229 | 0.543861 | 0.6990707 | 0.6846517 | 0.6502727 | 0.7174652 | 0.6808728 | 0.830375 |
| Phase-1 RCT-34 | 0.6387801 | 1.5861293 | 1.3183023 | 1.0276324 | 1.0787803 | 1.2954007 | 1.0674654 |
| Choline kinase | 0.874293 | 1.4916626 | 0.8799122 | 0.3803265 | 0.8254278 | 0.6908379 | 1.0818926 |
| Phase-1 RCT-20 | 0.7371658 | 0.8302786 | 0.8630465 | 0.8743954 | 0.8978444 | 0.8939894 | 0.9145553 |
| Phase-1 RCT-248 | 0.6712309 | 0.7836323 | 0.754254 | 0.8456698 | 0.9020482 | 0.8488089 | 0.9892739 |
| | BUS | BUS | CAD | CAD | CAD | CAD | CAD |
| Compound-Dose (2) | 14 | 14 | 1 | 1 | 1 | 4 | 4 |
| Animal Number (3) | 1742 | 1743 | 241 | 242 | 243 | 251 | 252 |
| Liver Toxicity Necrosis Classification (4) | no | no | no | no | no | yes | yes |
| Gene Name (5) | | | | | | | |
| Gadd153 | 1.0475538 | 1.0561299 | 0.9089944 | 0.735644 | 0.7706641 | 1.7784672 | 4.1913447 |
| Interferon related developmental regulator | 1.1515131 | 0.8593496 | 1.285831 | 2.18344 | 2.7619956 | 2.6053054 | 4.7076125 |
| IFRD1 (PC4) | | | | | | | |
| ID-1 | 1.1473962 | 0.9748331 | 1.0398078 | 1.4901352 | 0.8400593 | 1.6863209 | 1.5628446 |
| Insulin-like growth factor binding protein 1 | 1.0887092 | 1.2400824 | 1.1144817 | 5.54081 | 1.113687 | 7.2956443 | 5.4513335 |
| NIPK | 0.8863862 | 0.8994743 | 1.1472915 | 0.9823015 | 0.5546369 | 1.6890501 | 3.6218421 |
| Phase-1 RCT-50 | 0.8430437 | 0.8139131 | 1.1733599 | 1.3352906 | 0.9549855 | 4.944409 | 12.229047 |
| Sarcoplasmic reticulum calcium ATPase | 0.9695594 | 0.9030064 | 1.2706072 | 1.3421962 | 2.957581 | 0.678492 | 0.910686 |
| Phase-1 RCT-207 | 1.1249889 | 0.9853302 | 1.3360201 | 1.6967962 | 0.6133941 | 4.559005 | 3.5664513 |
| Acetyl-CoA carboxylase | 0.8183399 | 0.8851501 | 0.8708816 | 0.8644386 | 2.7955947 | 0.567617 | 0.6394426 |
| Cathepsin L, sequence 2 | 1.0815574 | 1.2412051 | 1.007254 | 1.480641 | 0.8483314 | 6.8919506 | 2.0798998 |
| Glucokinase | 1.3541161 | 1.0781056 | 0.604603 | 0.7297077 | 1.3890611 | 0.2688426 | 0.529699 |
| Gadd45 | 0.7636173 | 0.9979213 | 1.3960526 | 2.1730056 | 2.8994453 | 3.8760571 | 38.514282 |
| Phase-1 RCT-18 | 0.9069237 | 0.8260054 | 1.182789 | 0.9767414 | 1.2627156 | 0.5869311 | 1.0900828 |
| 3-beta-hydroxysteroid dehydrogenase | 1.1436836 | 0.9107903 | 1.1637231 | 1.2050658 | 0.5633227 | 1.0161211 | 0.8089499 |
| (HSD3B1) | | | | | | | |
| Phase-1 RCT-221 | 1.1359934 | 1.0450308 | 1.2387897 | 1.1090978 | 1.2215436 | 1.2531507 | 1.1069965 |
| Heme oxygenase | 0.9385045 | 0.9272289 | 3.2946606 | 3.054435 | 4.908183 | 47.470757 | 57.01253 |
| Cyclin dependent kinase 4 | 1.0054569 | 1.0423744 | 0.8879442 | 1.0582142 | 0.8220162 | 0.8813281 | 0.8955249 |
| Phase-1 RCT-75 | 0.9923911 | 1.031938 | 0.8751004 | 1.1153481 | 0.8657742 | 1.4018989 | 1.3910917 |
| Phase-1 RCT-65 | 1.0868341 | 1.1432387 | 0.9314526 | 0.6354988 | 0.3292022 | 1.3890532 | 1.667872 |
| Cytochrome P450 1A1 | 0.5989676 | 0.7000001 | 1.192324 | 0.7803829 | 0.6580517 | 1.0039625 | 1.0667142 |
| Peroxisomal multifunctional enzyme type II | 1.3255755 | 1.2721031 | 0.8158523 | 1.0111487 | 0.7199774 | 0.4682577 | 0.5266339 |
| DNA topoisomerase I | 1.0512486 | 1.1277868 | 1.1484349 | 2.0884538 | 3.715367 | 0.2958742 | 0.4297092 |
| Focal adhesion kinase (pp125FAK) | 0.8172565 | 0.8655612 | 1.1017176 | 0.9626966 | 1.9751055 | 1.0788057 | 0.8850282 |
| NGF-inducible anti-proliferative putative | 1.0441872 | 1.0161572 | 1.2893995 | 2.9619033 | 2.615093 | 1.6223496 | 2.42805 |
| secreted protein (PC3) | | | | | | | |
| Phase-1 RCT-82 | 0.9819508 | 0.8492837 | 1.0651476 | 0.686916 | 1.2597634 | 0.8827714 | 0.9895312 |
| Senescence marker protein-30 | 1.266858 | 1.3245778 | 0.585047 | 0.9921964 | 1.5621766 | 0.2999822 | 0.3345364 |
| Melanoma-associated antigen ME491 | 0.8630552 | 0.9617887 | 0.8536916 | 1.0143875 | 0.8925012 | 1.8828344 | 1.6821188 |
| Activating transcription factor 3 | 0.9100912 | 0.9018018 | 1.1096218 | 0.9853374 | 0.6316596 | 3.080833 | 25.819933 |
| Tryptophan hydroxylase | 1.0785295 | 1.015894 | 0.9535516 | 1.2077832 | 0.5629064 | 0.7340806 | 0.811272 |
| Zinc finger protein | 0.9160895 | 0.9568915 | 1.558597 | 2.37883 | 0.8517192 | 5.4700594 | 21.583532 |
| c-myc | 0.8112546 | 0.9711506 | 1.0993483 | 0.9602593 | 1.2759346 | 3.9707828 | 2.682938 |
| Cholesterol 7-alpha-hydroxylase (P450 | 0.8885627 | 0.7442218 | 1.1211157 | 0.8864611 | 3.253094 | 0.6640719 | 1.6600415 |
| VII) | | | | | | | |
| Ferritin H-chain | 0.9040417 | 1.0907272 | 0.8374969 | 0.8484959 | 0.8538694 | 2.082377 | 1.055599 |
| Phase-1 RCT-197 | 1.0120059 | 0.9611756 | 1.2368873 | 1.00115 | 1.2825346 | 0.9662318 | 1.3695793 |
| Phase-1 RCT-179 | 1.1987152 | 0.9695552 | 1.1152828 | 1.6270827 | 1.8402045 | 2.128932 | 1.681951 |
| Phase-1 RCT-15 | 1.1552873 | 1.3121269 | 1.1872265 | 1.4264657 | 0.6117948 | 1.275288 | 1.7276384 |
| Argininosuccinate lyase | 1.9772974 | 1.6830827 | 1.2270243 | 1.1070614 | 0.6138931 | 2.31668 | 1.969421 |
| Phase-1 RCT-49 | 0.7908649 | 0.8149074 | 1.5006728 | 1.5669149 | 2.813406 | 3.1739488 | 2.3326297 |
| Carbonic anhydrase III | 0.6858951 | 0.6850939 | 0.3161832 | 0.4799587 | 0.7679328 | 0.1421211 | 0.2213954 |
| Proliferating cell nuclear antigen gene | 0.7930945 | 0.9126682 | 0.8976878 | 0.8069082 | 1.6371257 | 1.5708321 | 1.4997784 |
| Integrin beta1 | 1.0174257 | 1.1432487 | 1.212915 | 2.9342427 | 1.0956092 | 3.5956244 | 2.739436 |
| Caspase 6 | 1.072487 | 1.1204032 | 0.9114535 | 1.2072804 | 0.8295605 | 0.9463219 | 1.3151512 |
| Phase-1 RCT-116 | 1.5817039 | 1.86618 | 0.8154889 | 0.6117767 | 0.2284352 | 0.8749169 | 0.8281035 |
| Phase-1 RCT-109 | 1.3647779 | 1.2901598 | 1.5504164 | 1.9094386 | 2.6704357 | 2.1494477 | 1.6760525 |
| Phase-1 RCT-71 | 1.2372489 | 1.244086 | 1.131043 | 1.2610104 | 1.1356494 | 1.2972516 | 1.6733831 |
| Elongation factor-1 alpha | 1.2120681 | 1.40359 | 0.7525337 | 1.1466509 | 2.3427007 | 1.2267385 | 1.4093816 |
| Phase-1 RCT-169 | 0.6498528 | 0.6190476 | 1.6728776 | 1.6617903 | 0.9157858 | 1.3354717 | 1.2033993 |
| Pyruvate kinase, muscle | 0.8843176 | 0.9863109 | 1.0171237 | 1.1107763 | 0.6613917 | 1.871353 | 1.79406 |
| Nucleosome assembly protein | 0.7910165 | 0.8329759 | 0.8888343 | 1.1807082 | 1.1388469 | 0.848704 | 0.8236355 |
| Phase-1 RCT-127 | 0.7129249 | 0.7880089 | 1.3186321 | 1.3102024 | 2.1354961 | 1.7621762 | 1.9327446 |
| Iron-responsive element-binding protein | 1.0525795 | 1.2087111 | 0.714421 | 0.6698155 | 1.0867572 | 0.4641822 | 0.4961864 |
| Phase-1 RCT-72 | 0.691988 | 0.7559106 | 1.1589519 | 0.8688329 | 0.6788448 | 1.9189606 | 1.605539 |
| Phase-1 RCT-144 | 0.9233254 | 0.9387069 | 1.2140448 | 0.9731309 | 1.7107127 | 3.512054 | 1.8008361 |
| Phase-1 RCT-242 | 0.8083488 | 0.8064815 | 2.2416735 | 2.0840216 | 1.8131837 | 2.3085923 | 3.3836668 |
| 17-beta hydroxysteroid dehydrogenase, | 0.4368525 | 0.9344581 | 0.6183488 | 0.9407536 | 0.8694136 | 0.8697096 | 0.5174992 |
| type 2 | | | | | | | |
| Phase-1 RCT-70 | 0.9319589 | 0.8628318 | 1.0814879 | 0.8755 | 1.0160475 | 2.0784848 | 1.7446764 |
| Ribosomal protein L13A | 1.2334831 | 1.2403561 | 1.1727208 | 1.4934385 | 1.6134553 | 1.7786049 | 2.062789 |
| Cytochrome P450 2E1 | 1.0771118 | 1.0624764 | 0.7063115 | 0.4193142 | 0.7208381 | 0.3589626 | 0.6163533 |
| AT-3 | 0.6480715 | 0.6905276 | 1.1708971 | 0.5802496 | 1.2058916 | 0.9047967 | 1.2390915 |
| Phase-1 RCT-270 | 1.0800492 | 1.2271212 | 0.5284225 | 0.849377 | 1.4046017 | 0.2892452 | 0.3067927 |
| Phase-1 RCT-123 | 0.8345833 | 0.8444109 | 1.1078539 | 0.8490867 | 0.8639182 | 0.9863658 | 0.862115 |
| Alpha-2-macroglobulin, sequence 2 | 1.0855992 | 0.9521127 | 1.1497104 | 2.000779 | 1.2809606 | 1.7354616 | 1.7171525 |
| Matrix metalloproteinase-1 | 0.9468756 | 1.1787173 | 0.8902082 | 1.0664856 | 1.2098839 | 2.2192895 | 1.0873777 |
| Beta-tubulin, class I | 1.6485544 | 1.6386973 | 0.7283505 | 0.4838234 | 0.512897 | 1.5877714 | 1.20785 |
| Phase-1 RCT-161 | 0.8704148 | 0.7769789 | 0.4752448 | 0.5530057 | 0.4297832 | 0.3533443 | 0.4025698 |
| 14-3-3 zeta | 0.9998972 | 1.2699002 | 0.9209735 | 1.1326066 | 0.7193997 | 1.9147629 | 2.0993452 |
| Stem cell factor | 1.0968151 | 1.0801704 | 0.7608326 | 0.6579562 | 0.5797728 | 0.7194997 | 0.7328701 |
| Macrophage inflammatory protein-2 alpha | 1.0451177 | 0.9961158 | 2.0376859 | 2.638398 | 1.6586162 | 1.7633419 | 2.6699395 |
| Thymidylate synthase | 0.7807429 | 0.9003704 | 1.0457618 | 0.7898116 | 1.03352 | 0.8720765 | 1.0496972 |
| c-jun | 1.2097919 | 1.194697 | 1.2178226 | 0.9448812 | 0.6342033 | 1.6447593 | 8.064402 |
| Superoxide dismutase Mn | 1.2555023 | 1.2673516 | 1.1477357 | 1.4560213 | 2.5777514 | 1.7073054 | 1.4551935 |
| Phase-1 RCT-73 | 0.8923963 | 0.8530184 | 0.8766896 | 0.9333333 | 0.9139305 | 0.691861 | 0.7773256 |
| Macrophage inflammatory protein-1 alpha | 0.9977617 | 1.0650318 | 1.0718037 | 0.8590661 | 0.4559776 | 1.6225747 | 2.1227517 |
| Phase-1 RCT-214 | 1.223886 | 1.3733904 | 0.626689 | 0.6358515 | 1.058417 | 0.2745062 | 0.4583045 |
| NADH-cytochrome b5 reductase | 1.0547508 | 1.1920314 | 0.6494751 | 0.5463828 | 1.3806888 | 0.614027 | 0.4387768 |
| Interleukin-1 beta | 0.7699202 | 0.8281482 | 0.9755658 | 1.2756212 | 1.5582001 | 2.1154296 | 1.2801836 |
| NADP-dependent isocitrate | 1.1318537 | 1.1169341 | 0.7735624 | 0.9043602 | 1.4442937 | 0.766187 | 0.6230809 |
| dehydrogenase, cytosolic | | | | | | | |
| Phase-1 RCT-289 | 0.9632787 | 0.9539801 | 0.808135 | 1.0841353 | 1.1210116 | 0.7338429 | 0.7136167 |
| Glutamine synthetase | 0.8764148 | 1.2538844 | 0.7621953 | 1.0604109 | 1.8305466 | 0.5388102 | 0.5860333 |
| Phase-1 RCT-182 | 1.0772256 | 1.0673342 | 0.7963047 | 0.7824888 | 1.7193425 | 0.4853855 | 0.4681138 |
| Ubiquitin conjugating enzyme (RAD 6 | 0.9698472 | 1.1446311 | 1.062068 | 1.5463047 | 1.3830999 | 1.1966028 | 1.8469709 |
| homologue) | | | | | | | |
| Extracellular-signal-regulated kinase 1 | 1.0059168 | 1.0007262 | 0.8355094 | 0.7028729 | 0.3836779 | 0.731225 | 0.6008956 |
| Emerin | 0.9191657 | 0.886896 | 0.9432033 | 1.1907893 | 0.766429 | 0.8219273 | 0.9654654 |
| Phase-1 RCT-78 | 0.9589797 | 1.015894 | 0.9827404 | 0.8378817 | 1.3197054 | 0.8066924 | 0.8176216 |
| Phase-1 RCT-212 | 0.9722207 | 0.958231 | 1.046214 | 0.8155718 | 0.7014004 | 1.0733169 | 0.9886604 |
| Phase-1 RCT-168 | 0.966176 | 1.1560237 | 0.6844505 | 0.7132889 | 1.5763406 | 0.5036778 | 0.591979 |
| Lysyl hydroxylase | 0.9587117 | 1.0352231 | 0.7997726 | 0.9859111 | 1.1686845 | 0.7328628 | 0.8008592 |
| Phase-1 RCT-55 | 0.6204104 | 0.6223261 | 0.963482 | 0.7976606 | 0.6819216 | 1.5112758 | 0.9790343 |
| MHC class I antigen RT1.A1(f) alpha- | 1.2983035 | 1.1754553 | 1.0189772 | 0.5579769 | 0.2741749 | 1.6154394 | 1.1893224 |
| chain | | | | | | | |
| Phase-1 RCT-40 | 0.9933025 | 1.2809854 | 0.7026357 | 1.2740551 | 1.8200084 | 0.5969545 | 0.5337219 |
| Cyclin G | 0.944012 | 1.1094241 | 1.1097989 | 1.5083688 | 1.2410102 | 4.528601 | 2.3966677 |
| Peroxisome proliferator activated receptor | 0.8392525 | 0.8190694 | 1.058116 | 0.6842327 | 0.6390063 | 0.9299547 | 1.3657857 |
| gamma | | | | | | | |
| Phase-1 RCT-280 | 0.9335378 | 0.8376858 | 1.3125235 | 1.1449205 | 0.8824546 | 0.6763532 | 0.773286 |
| D-dopachrome tautomerase | 1.0794488 | 1.1520737 | 0.6796878 | 0.6452309 | 1.49937 | 0.6775488 | 0.4741986 |
| Phase-1 RCT-287 | 1.1376208 | 1.0940928 | 0.8337989 | 1.0691193 | 0.9145294 | 0.4194513 | 0.7038358 |
| Phase-1 RCT-119 | 0.797407 | 0.8226866 | 1.0986946 | 1.0674233 | 1.0431154 | 0.6955313 | 0.7514412 |
| Thioredoxin-1 (Trx1) | 0.9958323 | 1.0312477 | 1.1465044 | 1.8762884 | 2.3035288 | 2.1524622 | 1.6517862 |
| Phase-1 RCT-225 | 1.2174518 | 1.1149601 | 1.2236041 | 0.8541667 | 1.0149516 | 7.5371675 | 8.013704 |
| Protein kinase C alpha | 0.9058563 | 0.8865792 | 0.8792956 | 0.8532117 | 2.1292083 | 0.8836915 | 0.9123103 |
| Phase-1 RCT-87 | 1.028194 | 1.0208054 | 0.8902062 | 0.906773 | 1.4464972 | 0.5573033 | 0.7969732 |
| Phase-1 RCT-59 | 1.1663467 | 0.8516667 | 1.3083559 | 1.9628078 | 0.8895317 | 2.1095455 | 3.1501558 |
| C4b-binding protein | 0.7859813 | 0.8804129 | 1.1052866 | 0.9902369 | 0.9963656 | 0.5723452 | 0.4712398 |
| Phase-1 RCT-204 | 0.8714277 | 0.9083083 | 1.0133529 | 0.8772284 | 0.8086446 | 1.2025608 | 0.8403303 |
| Carnitine palmitoyl-CoA transferase | 1.2962945 | 0.9913523 | 1.0922921 | 0.6705608 | 0.4183972 | 1.1406351 | 1.7792885 |
| 8-oxoguanine DNA glycosylase | 0.6865799 | 0.7590508 | 0.9568839 | 0.7817398 | 0.8435737 | 0.7359022 | 1.011185 |
| H-rev107 | 0.853678 | 0.8700699 | 0.7340477 | 0.6326667 | 0.8259419 | 0.6086437 | 0.6959423 |
| Preproalbumin | 1.2445313 | 1.2092645 | 0.6864559 | 1.1100718 | 0.321097 | 0.5361729 | 0.4756639 |
| Phase-1 RCT-177 | 1.2103966 | 1.1801634 | 0.7705215 | 1.1012383 | 0.5297067 | 0.5155912 | 0.6399711 |
| Phase-1 RCT-110 | 0.7637737 | 0.7626097 | 1.0459318 | 0.8761396 | 0.8461012 | 1.022452 | 1.1059798 |
| Phase-1 RCT-227 | 1.1182265 | 1.086273 | 0.9499276 | 1.1763122 | 0.9910691 | 0.8043385 | 0.682657 |
| Neuropeptide Y | 0.7919618 | 0.745768 | 1.0361329 | 0.910638 | 1.3242875 | 1.5928048 | 1.8869846 |
| Phase-1 RCT-277 | 0.8494842 | 0.9089431 | 0.8340284 | 1.003982 | 1.1922239 | 0.6185063 | 0.6866697 |
| Phase-1 RCT-229 | 0.78214 | 0.7511112 | 1.1212664 | 0.704383 | 0.6961917 | 1.0038159 | 1.1605916 |
| Phase-1 RCT-34 | 0.9526011 | 1.2105337 | 1.2349437 | 1.1032833 | 1.520847 | 8.251641 | 6.081258 |
| Choline kinase | 1.6725014 | 1.0950981 | 1.3472157 | 0.9203776 | 0.4187047 | 3.7908692 | 6.931563 |
| Phase-1 RCT-20 | 0.8375808 | 0.8401632 | 1.043185 | 0.7252298 | 0.6511919 | 1.3701224 | 1.9221274 |
| Phase-1 RCT-248 | 0.7937995 | 0.8284314 | 1.3055539 | 0.9568236 | 1.1289353 | 1.5496231 | 1.2461208 |
| | CCL4 | CCL4 | CCL4 | CCL4 | CCL4 | CCL4 | CAR |
| Compound-Dose (2) | 250 | 250 | 250 | 1000 | 1000 | 1000 | 16 |
| Animal Number (3) | 2041 | 2042 | 2043 | 2051 | 2052 | 2053 | 1851 |
| Liver Toxicity Necrosis Classification (4) | yes | yes | yes | yes | yes | yes | no |
| Gene Name (5) | | | | | | | |
| Gadd153 | 2.336987 | 2.0631466 | 2.100735 | 2.4715695 | 2.707997 | 2.8786454 | 1.2246268 |
| Interferon related developmental regulator | 2.105227 | 2.2468853 | 2.0867763 | 1.5571995 | 2.5576532 | 2.3199174 | 0.8265826 |
| IFRD1 (PC4) | | | | | | | |
| ID-1 | 1.5316503 | 1.5534334 | 1.3917205 | 1.5481912 | 1.469856 | 1.9059677 | 1.3212638 |
| Insulin-like growth factor binding protein 1 | 4.720753 | 4.2272696 | 5.3301377 | 8.427773 | 11.837842 | 11.806425 | 0.8553523 |
| NIPK | 1.6963229 | 1.717954 | 1.576483 | 1.5700719 | 1.9555297 | 1.6383938 | 1.3981199 |
| Phase-1 RCT-50 | 1.7896957 | 2.0087337 | 1.828204 | 2.6136935 | 3.4895608 | 3.386395 | 1.202367 |
| Sarcoplasmic reticulum calcium ATPase | 1.2678341 | 1.4873061 | 1.3692182 | 1.1542474 | 1.2307341 | 1.3435622 | 0.8193305 |
| Phase-1 RCT-207 | 2.2170343 | 2.5764434 | 2.5619826 | 4.3550987 | 4.160575 | 4.602809 | 1.5319546 |
| Acetyl-CoA carboxylase | 0.9381389 | 0.9414856 | 0.9978434 | 0.7945885 | 0.9017361 | 0.9188139 | 1.0308716 |
| Cathepsin L, sequence 2 | 2.9562008 | 3.3319163 | 2.4368432 | 6.2943854 | 5.4248834 | 4.533411 | 0.7550266 |
| Glucokinase | 0.727991 | 0.7952781 | 0.790524 | 0.842413 | 0.9431188 | 0.7237573 | 0.6362357 |
| Gadd45 | 2.6577005 | 3.1833534 | 2.184613 | 3.8200366 | 7.0446534 | 5.078827 | 0.8816645 |
| Phase-1 RCT-18 | 0.8971551 | 0.9166572 | 0.9115502 | 0.7632367 | 0.8317633 | 0.7923557 | 0.9938912 |
| 3-beta-hydroxysteroid dehydrogenase | 0.7855619 | 0.9304191 | 0.7794742 | 0.7549982 | 0.8669117 | 0.725601 | 0.9776313 |
| (HSD3B1) | | | | | | | |
| Phase-1 RCT-221 | 0.9673877 | 0.9991572 | 1.0352503 | 1.0309238 | 0.9353919 | 0.9911338 | 0.9580409 |
| Heme oxygenase | 7.2549453 | 7.2823186 | 5.9417424 | 15.42959 | 18.257088 | 14.647765 | 1.0759474 |
| Cyclin dependent kinase 4 | 1.0841067 | 1.1992817 | 1.1343513 | 1.1669427 | 1.3328245 | 1.1981064 | 0.9250284 |
| Phase-1 RCT-75 | 1.4566525 | 1.2356762 | 1.3514895 | 1.3709408 | 1.5162334 | 1.6612226 | 0.81956 |
| Phase-1 RCT-65 | 1.2702037 | 1.3508801 | 1.4941704 | 1.3753192 | 1.3057662 | 1.4137537 | 1.5858599 |
| Cytochrome P450 1A1 | 1.1083431 | 1.0099761 | 1.0265131 | 1.3231844 | 1.3516355 | 1.3439691 | 1.1423639 |
| Peroxisomal multifunctional enzyme type II | 0.7733123 | 0.8140461 | 0.7630166 | 0.6367751 | 0.7406758 | 0.6773458 | 0.7015513 |
| DNA topoisomerase I | 0.9159226 | 0.8155692 | 0.9852278 | 0.5800715 | 0.6557872 | 0.5850375 | 0.7523254 |
| Focal adhesion kinase (pp125FAK) | 1.0165051 | 0.9772694 | 0.9479876 | 1.0883276 | 1.161601 | 1.1856351 | 0.8622747 |
| NGF-inducible anti-proliferative putative | 0.9585678 | 1.117477 | 0.9117649 | 0.9562724 | 1.4657005 | 1.2530751 | 0.6525267 |
| secreted protein (PC3) | | | | | | | |
| Phase-1 RCT-82 | 0.8971776 | 0.9430782 | 0.9651775 | 0.7957453 | 0.8697621 | 0.7596626 | 0.9901292 |
| Senescence marker protein-30 | 0.3201941 | 0.4511159 | 0.3589867 | 0.2782697 | 0.2601456 | 0.2436091 | 0.7016908 |
| Melanoma-associated antigen ME491 | 1.2693484 | 1.5421274 | 1.0887313 | 1.4460311 | 1.3368099 | 1.4273763 | 1.050204 |
| Activating transcription factor 3 | 1.7783788 | 1.4138048 | 1.3087331 | 1.7502118 | 1.6803215 | 2.788536 | 1.3956591 |
| Tryptophan hydroxylase | 0.8387789 | 0.8976374 | 0.9079463 | 0.7880402 | 0.9833096 | 0.7841834 | 0.7815464 |
| Zinc finger protein | 1.2471575 | 1.1353724 | 1.2073545 | 2.20005 | 2.4226031 | 4.3565836 | 1.0865802 |
| c-myc | 1.982732 | 2.031786 | 2.044644 | 3.0200953 | 3.5158072 | 4.6503835 | 1.0891727 |
| Cholesterol 7-alpha-hydroxylase (P450 | 0.5193926 | 0.7015054 | 0.566205 | 0.8267121 | 0.7317732 | 0.5285978 | 0.9567298 |
| VII) | | | | | | | |
| Ferritin H-chain | 1.5942243 | 1.9397582 | 1.5407901 | 3.0893784 | 2.66429 | 3.2831922 | 0.7372654 |
| Phase-1 RCT-197 | 1.7041267 | 1.880463 | 1.815517 | 2.1445417 | 2.618018 | 2.3513167 | 1.4282553 |
| Phase-1 RCT-179 | 1.9652004 | 1.9016367 | 1.7535436 | 3.1131349 | 2.7610462 | 2.7161212 | 1.1183516 |
| Phase-1 RCT-15 | 1.5455071 | 1.7218494 | 1.7525623 | 1.8089906 | 2.2995114 | 2.307848 | 1.378844 |
| Argininosuccinate lyase | 2.4241266 | 2.0160434 | 2.0917354 | 1.6834902 | 2.2397385 | 2.3092072 | 1.0036882 |
| Phase-1 RCT-49 | 1.876507 | 1.7790385 | 1.5383307 | 2.3413782 | 2.3936243 | 2.238492 | 1.0868777 |
| Carbonic anhydrase III | 0.1738059 | 0.3684128 | 0.3301551 | 0.1636487 | 0.1863654 | 0.1882683 | 0.6906969 |
| Proliferating cell nuclear antigen gene | 1.3992065 | 1.4459836 | 1.3589199 | 1.7917043 | 1.9668527 | 1.9949214 | 0.9559984 |
| Integrin beta1 | 5.58679 | 5.637676 | 5.575098 | 11.639049 | 13.900116 | 14.06068 | 1.030491 |
| Caspase 6 | 3.4051418 | 3.962217 | 3.0601647 | 7.7323384 | 7.8128324 | 6.7494555 | 1.0213523 |
| Phase-1 RCT-116 | 1.2295191 | 1.3800625 | 1.3761586 | 1.4826359 | 1.2863501 | 1.2546318 | 2.9379618 |
| Phase-1 RCT-109 | 1.3698624 | 1.2746289 | 1.2012031 | 1.5856453 | 1.4261839 | 1.3318579 | 0.8279164 |
| Phase-1 RCT-71 | 1.2983922 | 1.2536311 | 1.2558036 | 1.3095739 | 1.6219091 | 1.4555721 | 1.1963665 |
| Elongation factor-1 alpha | 1.4109732 | 1.5648483 | 1.4123816 | 1.6647105 | 1.6185483 | 1.7237053 | 0.7960954 |
| Phase-1 RCT-169 | 2.0839918 | 1.5128883 | 2.2824984 | 1.4098899 | 1.2869285 | 1.4033358 | 1.1969987 |
| Pyruvate kinase, muscle | 1.9241703 | 1.7307527 | 1.747322 | 3.4254837 | 3.286254 | 2.8736959 | 0.9783188 |
| Nucleosome assembly protein | 0.673108 | 0.5931408 | 0.9263877 | 0.4946106 | 0.4596022 | 0.5580892 | 1.2873944 |
| Phase-1 RCT-127 | 1.6482924 | 1.5943937 | 1.4628253 | 1.8463196 | 1.7971846 | 2.2165473 | 1.0937527 |
| Iron-responsive element-binding protein | 0.6346712 | 0.6280296 | 0.6875836 | 0.6063271 | 0.522027 | 0.5200608 | 0.7230943 |
| Phase-1 RCT-72 | 1.7562784 | 1.6021874 | 1.7512926 | 2.1262965 | 2.0971465 | 2.139062 | 0.9696162 |
| Phase-1 RCT-144 | 2.5060542 | 2.4134476 | 2.3163002 | 3.6047573 | 3.9221654 | 4.2120833 | 1.045583 |
| Phase-1 RCT-242 | 1.8941776 | 2.8681173 | 2.1463683 | 3.5348887 | 5.8996334 | 4.5911546 | 1.3688867 |
| 17-beta hydroxysteroid dehydrogenase, | 0.5885319 | 0.4408018 | 0.9135144 | 0.4176675 | 0.3611446 | 0.5247926 | 1.9652655 |
| type 2 | | | | | | | |
| Phase-1 RCT-70 | 1.3431274 | 1.365344 | 1.3367865 | 1.8658892 | 1.8379011 | 1.4796546 | 1.0849639 |
| Ribosomal protein L13A | 1.4366585 | 1.2794713 | 1.1710712 | 2.0106037 | 1.5940348 | 1.5394613 | 0.8054659 |
| Cytochrome P450 2E1 | 0.34987 | 0.5508418 | 0.5566751 | 0.459387 | 0.3523421 | 0.3069493 | 0.6867144 |
| AT-3 | 0.900798 | 0.928784 | 0.9036409 | 0.7521226 | 0.8791553 | 0.7845656 | 1.0431468 |
| Phase-1 RCT-270 | 0.4227928 | 0.5645848 | 0.601451 | 0.3714876 | 0.3518298 | 0.3335138 | 0.6905913 |
| Phase-1 RCT-123 | 0.9075536 | 0.9079689 | 0.9067525 | 0.7631707 | 0.8026587 | 0.8739291 | 1.0321673 |
| Alpha-2-macroglobulin, sequence 2 | 1.062175 | 1.2175983 | 1.086588 | 1.0451134 | 1.034116 | 0.9554697 | 0.6083725 |
| Matrix metalloproteinase-1 | 1.5778139 | 1.8503144 | 1.6198367 | 3.1427984 | 2.6413348 | 3.2151663 | 0.9343071 |
| Beta-tubulin, class I | 2.0626967 | 2.0077472 | 1.943834 | 2.4189525 | 2.8958285 | 2.3575835 | 1.1647443 |
| Phase-1 RCT-161 | 0.3406974 | 0.5150338 | 0.5795457 | 0.4771628 | 0.5360016 | 0.49534 | 1.2625333 |
| 14-3-3 zeta | 1.390145 | 1.366735 | 1.354112 | 1.8211747 | 1.6429375 | 1.9442023 | 1.7373629 |
| Stem cell factor | 0.4953751 | 0.7185977 | 0.6788662 | 0.4406641 | 0.4377355 | 0.3629124 | 0.9345752 |
| Macrophage inflammatory protein-2 alpha | 1.674307 | 1.7014763 | 1.8466084 | 2.519656 | 2.2259827 | 2.1930737 | 1.10924 |
| Thymidylate synthase | 0.6122141 | 0.5049558 | 0.5152879 | 0.5316984 | 0.5984195 | 0.6110783 | 1.1583619 |
| c-jun | 1.6744564 | 1.9502923 | 1.9512099 | 2.807757 | 2.347875 | 2.213561 | 2.3511446 |
| Superoxide dismutase Mn | 1.8537257 | 1.6785889 | 2.1623943 | 3.5460567 | 2.7466645 | 3.369079 | 1.0707496 |
| Phase-1 RCT-73 | 0.8184901 | 0.8025156 | 0.762677 | 0.6080071 | 0.6664004 | 0.662912 | 0.8525065 |
| Macrophage inflammatory protein-1 alpha | 1.6662898 | 1.6200022 | 1.8087444 | 1.727355 | 1.4408935 | 1.6883032 | 1.4175925 |
| Phase-1 RCT-214 | 0.6621282 | 0.7203413 | 0.6400244 | 0.4267588 | 0.5187004 | 0.4137026 | 1.0126282 |
| NADH-cytochrome b5 reductase | 0.7332569 | 0.645074 | 0.772957 | 0.6040322 | 0.5932336 | 0.5836774 | 0.6994225 |
| Interleukin-1 beta | 1.7234089 | 1.9688027 | 1.7254527 | 3.1537135 | 2.6244602 | 3.354438 | 0.9295259 |
| NADP-dependent isocitrate | 0.7113699 | 0.7278209 | 0.711077 | 0.4627577 | 0.5217453 | 0.3940006 | 0.6639713 |
| dehydrogenase, cytosolic | | | | | | | |
| Phase-1 RCT-289 | 0.75779 | 0.7900196 | 0.8038961 | 0.4963911 | 0.5438005 | 0.5764203 | 0.8041027 |
| Glutamine synthetase | 0.491175 | 0.5321041 | 0.4565988 | 0.679283 | 0.6095827 | 0.5910178 | 0.6528148 |
| Phase-1 RCT-182 | 0.4775423 | 0.6649774 | 0.6687151 | 0.5583551 | 0.4173144 | 0.3903699 | 0.7850449 |
| Ubiquitin conjugating enzyme (RAD 6 | 0.9807192 | 1.4882606 | 1.1661017 | 1.9029436 | 1.7174611 | 1.635866 | 1.0578318 |
| homologue) | | | | | | | |
| Extracellular-signal-regulated kinase 1 | 0.4273931 | 0.6328296 | 0.5707476 | 0.3755559 | 0.3917442 | 0.39774 | 0.84795 |
| Emerin | 0.984218 | 0.949385 | 0.9778266 | 0.7417402 | 0.8882057 | 0.7879438 | 1.0575026 |
| Phase-1 RCT-78 | 0.7445614 | 0.8847877 | 0.8493385 | 0.6596265 | 0.675832 | 0.6908882 | 1.0651649 |
| Phase-1 RCT-212 | 1.5980073 | 1.3786545 | 1.8949147 | 4.0689783 | 2.5560122 | 2.3504655 | 1.2218957 |
| Phase-1 RCT-168 | 0.8004694 | 0.946668 | 0.900247 | 0.7805905 | 0.9129972 | 0.8111624 | 0.7030972 |
| Lysyl hydroxylase | 0.6494552 | 0.6633485 | 0.6113004 | 0.7364157 | 0.72148 | 0.718063 | 0.7185187 |
| Phase-1 RCT-55 | 1.4592545 | 1.6486949 | 1.8197386 | 2.0616403 | 2.0897963 | 2.536242 | 0.9593602 |
| MHC class I antigen RT1.A1(f) alpha- | 1.8027383 | 1.5763335 | 2.1896572 | 2.2453513 | 2.1532924 | 1.9826437 | 2.1449192 |
| chain | | | | | | | |
| Phase-1 RCT-40 | 0.7205404 | 0.707553 | 0.8111039 | 0.5432568 | 0.5892958 | 0.5492194 | 0.6229137 |
| Cyclin G | 1.5011126 | 1.5073917 | 1.5719965 | 2.348219 | 2.5908928 | 3.1865892 | 2.723597 |
| Peroxisome proliferator activated receptor | 0.6646236 | 0.5765904 | 0.5458802 | 0.4515298 | 0.5421776 | 0.4926039 | 1.3089945 |
| gamma | | | | | | | |
| Phase-1 RCT-280 | 0.6999338 | 0.8476288 | 0.6449291 | 0.4056287 | 0.5974889 | 0.5361142 | 1.060417 |
| D-dopachrome tautomerase | 0.6205115 | 0.6639308 | 0.6627238 | 0.523486 | 0.5032601 | 0.5527148 | 0.6528705 |
| Phase-1 RCT-287 | 0.8884646 | 0.9417581 | 0.8268098 | 0.6728452 | 0.815438 | 0.6836649 | 0.6799559 |
| Phase-1 RCT-119 | 0.77737 | 0.9768846 | 1.1320573 | 0.448792 | 0.4975044 | 0.5205638 | 0.5668562 |
| Thioredoxin-1 (Trx1) | 1.4850017 | 1.5040743 | 1.3818958 | 1.9179963 | 1.8905054 | 1.745985 | 0.7870935 |
| Phase-1 RCT-225 | 3.3056962 | 2.274617 | 3.7255003 | 8.37507 | 6.0022345 | 5.58821 | 1.9433713 |
| Protein kinase C alpha | 0.9899713 | 1.1077287 | 1.061545 | 1.1903932 | 1.1750414 | 1.1700243 | 1.045463 |
| Phase-1 RCT-87 | 1.1588528 | 0.9832309 | 1.0200373 | 0.8281702 | 0.9573354 | 0.9298392 | 0.7887006 |
| Phase-1 RCT-59 | 1.0576165 | 1.0359329 | 0.940391 | 0.8151788 | 1.0401115 | 1.1751164 | 1.6581818 |
| C4b-binding protein | 0.7300496 | 0.7454422 | 0.7488737 | 0.5805854 | 0.6723028 | 0.6336272 | 0.8072755 |
| Phase-1 RCT-204 | 0.8643243 | 0.8817229 | 0.9129091 | 0.8214892 | 0.7521097 | 0.7524658 | 1.0048233 |
| Carnitine palmitoyl-CoA transferase | 0.8990353 | 0.9224328 | 0.8629154 | 0.444256 | 0.4823133 | 0.4751923 | 2.0136456 |
| 8-oxoguanine DNA glycosylase | 1.0607635 | 0.8456528 | 0.8804722 | 0.8724565 | 0.931313 | 1.081931 | 1.0713483 |
| H-rev107 | 0.916499 | 0.7918578 | 0.8462916 | 0.8757867 | 0.9324182 | 0.9368123 | 0.9915804 |
| Preproalbumin | 0.8304126 | 1.1711377 | 0.7312804 | 0.4071997 | 0.8350648 | 0.7280483 | 1.5836138 |
| Phase-1 RCT-177 | 0.7908749 | 0.8189223 | 0.7508913 | 0.6516799 | 0.6237009 | 0.6327945 | 0.7695812 |
| Phase-1 RCT-110 | 0.9430677 | 0.958835 | 0.9506849 | 0.8843915 | 0.9034989 | 0.9026997 | 1.1207397 |
| Phase-1 RCT-227 | 0.6760763 | 0.9568358 | 0.8134082 | 0.6889421 | 0.6528188 | 0.7341505 | 1.0027298 |
| Neuropeptide Y | 1.0144942 | 1.0965065 | 1.0413741 | 0.9498293 | 1.0001891 | 0.9964304 | 1.0656506 |
| Phase-1 RCT-277 | 0.9223269 | 0.7593156 | 0.7696189 | 0.4979741 | 0.5991657 | 0.764937 | 0.7127979 |
| Phase-1 RCT-229 | 0.9155199 | 0.9426535 | 0.9398429 | 0.8226131 | 0.8385363 | 0.839009 | 1.0952379 |
| Phase-1 RCT-34 | 1.5896841 | 2.4088078 | 3.02467 | 2.300364 | 2.348732 | 3.389625 | 1.4910659 |
| Choline kinase | 1.3510892 | 1.6978487 | 1.3622925 | 1.4661915 | 1.768377 | 1.7108403 | 1.1193753 |
| Phase-1 RCT-20 | 1.340057 | 1.4144884 | 1.3475461 | 1.4472241 | 1.508543 | 1.3992178 | 1.0838178 |
| Phase-1 RCT-248 | 1.1528746 | 1.1174567 | 1.0723118 | 1.3250748 | 1.2372736 | 1.1092035 | 0.9228208 |
| | CAR | CAR | CHCL3 | CHCL3 | CHCL3 | CHCL3 | CHLOR |
| Compound-Dose (2) | 16 | 16 | 250 | 250 | 250 | 500 | 8 |
| Animal Number (3) | 1852 | 1853 | 1621 | 1622 | 1623 | 2352 | 41 |
| Liver Toxicity Necrosis Classification (4) | no | no | no | no | no | no | no |
| Gene Name (5) | | | | | | | |
| Gadd153 | 1 | 1.2502046 | 1.1471001 | 1.0448989 | 1.0678427 | 1.5527178 | 1.2702795 |
| Interferon related developmental regulator | 0.8141601 | 0.8941247 | 1.5660272 | 1.1251776 | 1.3328296 | 1.3661786 | 1.0549568 |
| IFRD1 (PC4) | | | | | | | |
| ID-1 | 1.1135905 | 1.2173277 | 1.088379 | 1.0999694 | 1.0878427 | 0.9680817 | 1.0950811 |
| Insulin-like growth factor binding protein 1 | 0.9057186 | 1.2206831 | 1.616715 | 2.02622 | 1.9173236 | 3.7368171 | 0.8721584 |
| NIPK | 1.2716104 | 1.4406834 | 1.5155158 | 1.2350659 | 1.3266516 | 2.5291088 | 1.2752138 |
| Phase-1 RCT-50 | 1.2890792 | 1.1351724 | 1.2189993 | 1.1384875 | 1.0837743 | 1.2461538 | 0.978 2063 |
| Sarcoplasmic reticulum calcium ATPase | 0.9105485 | 0.9407387 | 1.0431494 | 0.9994877 | 1.0213084 | 0.9494347 | 0.6381649 |
| Phase-1 RCT-207 | 1.3697987 | 1.3565191 | 1.565766 | 1.6328045 | 1.5113041 | 1.4598675 | 0.9311277 |
| Acetyl-CoA carboxylase | 0.8355119 | 0.7546511 | 0.809708 | 0.8167129 | 0.8775639 | 0.8896514 | 1.1751372 |
| Cathepsin L, sequence 2 | 0.9941311 | 1.1112007 | 1.8517196 | 1.2871497 | 1.9157559 | 1.7415808 | 1.0761861 |
| Glucokinase | 0.4607391 | 0.4305266 | 0.6858354 | 0.6675334 | 0.5827413 | 0.6920635 | 0.882147 |
| Gadd45 | 0.8008952 | 0.912503 | 1.8503075 | 1.7872658 | 1.7578572 | 1.7833834 | 2.1535591 |
| Phase-1 RCT-18 | 1.0472413 | 0.9703851 | 0.8542507 | 0.9330775 | 0.8650262 | 0.9352822 | 1.1701256 |
| 3-beta-hydroxysteroid dehydrogenase | 0.9086611 | 0.9584301 | 0.6587421 | 0.8629249 | 0.866856 | 0.8566523 | 1.3470904 |
| (HSD3B1) | | | | | | | |
| Phase-1 RCT-221 | 0.9612153 | 1.095486 | 0.995846 | 0.9702906 | 0.9977396 | 1.1619221 | 1.2151778 |
| Heme oxygenase | 1.1849327 | 1.1638148 | 6.460615 | 5.769106 | 4.549837 | 6.173422 | 1.1170713 |
| Cyclin dependent kinase 4 | 0.9400246 | 1.1559956 | 1.0587443 | 0.9316079 | 0.9801806 | 0.7877874 | 1.1760414 |
| Phase-1 RCT-75 | 0.9203378 | 0.9425129 | 1.3855288 | 0.8913118 | 1.0239129 | 1.0770934 | 1.1891619 |
| Phase-1 RCT-65 | 1.502033 | 1.6688019 | 1.2830735 | 1.0583618 | 1.1322846 | 0.9264495 | 1.1280415 |
| Cytochrome P450 1A1 | 1.280067 | 0.9238569 | 0.9291704 | 1.1437533 | 0.9454091 | 1.0220801 | 0.9075969 |
| Peroxisomal multifunctional enzyme type II | 0.7502904 | 1.0430362 | 0.7776327 | 0.9754915 | 1.0788783 | 1.0161079 | 0.9068809 |
| DNA topoisomerase I | 0.7558974 | 0.8580351 | 1.6547451 | 0.7431991 | 0.6429116 | 0.7267783 | 1.5987933 |
| Focal adhesion kinase (pp125FAK) | 0.9659454 | 0.8885287 | 0.9999507 | 0.969007 | 1.0027155 | 1.0479146 | 0.9528391 |
| NGF-inducible anti-proliferative putative | 0.6436703 | 0.7393819 | 1.0062159 | 0.8940088 | 1.0879629 | 0.9682658 | 0.8795686 |
| secreted protein (PC3) | | | | | | | |
| Phase-1 RCT-82 | 1.0227041 | 0.9167779 | 0.9452336 | 0.9776212 | 0.97824 | 0.877224 | 1.072274 |
| Senescence marker protein-30 | 0.8285546 | 0.9557829 | 0.3805252 | 0.4592952 | 0.5463058 | 0.2292552 | 0.4954177 |
| Melanoma-associated antigen ME491 | 0.9050299 | 1.0901425 | 1.0815402 | 1.0290729 | 0.921649 | 0.9296494 | 0.9012038 |
| Activating transcription factor 3 | 1.3707942 | 1.0578363 | 1.1613007 | 1.2657187 | 1.0803897 | 1.9803959 | 1.7989016 |
| Tryptophan hydroxylase | 0.818873 | 0.7946968 | 1.0855227 | 1.0740151 | 1.0610945 | 0.8214706 | 1.1305271 |
| Zinc finger protein | 1.103625 | 0.9460855 | 1.679381 | 1.4726762 | 1.4159324 | 2.2407913 | 1.0152197 |
| c-myc | 1.1847197 | 1.1106578 | 1.5296278 | 1.2490377 | 1.2753673 | 2.9705229 | 1.2371854 |
| Cholesterol 7-alpha-hydroxylase (P450 | 1.1698875 | 0.9348938 | 0.8170714 | 0.8214194 | 0.7448636 | 0.6468221 | 0.8935267 |
| VII) | | | | | | | |
| Ferritin H-chain | 0.7863495 | 1.0734227 | 1.016603 | 0.7542932 | 1.187773 | 1.0238458 | 0.9 2223 |
| Phase-1 RCT-197 | 1.2697457 | 1.2926725 | 1.3312808 | 1.0529103 | 1.0771221 | 1.173303 | 1.0729241 |
| Phase-1 RCT-179 | 1.1392597 | 1.6448033 | 1.0510439 | 1.1152711 | 1.1304893 | 1.4103645 | 0.9855346 |
| Phase-1 RCT-15 | 1.5423017 | 1.4481926 | 1.2144095 | 1.205564 | 1.1498176 | 1.0040804 | 1.4195181 |
| Argininosuccinate lyase | 1.1576478 | 1.3805453 | 1.2380421 | 1.6035173 | 1.6042439 | 1.17999 | 1.1209998 |
| Phase-1 RCT-49 | 1.0586914 | 1.1268256 | 1.268598 | 1.0799574 | 1.3838192 | 1.599215 | 0.9956963 |
| Carbonic anhydrase III | 0.6334651 | 0.4966585 | 0.1767803 | 0.2984954 | 0.4036249 | 0.1881159 | 0.5921557 |
| Proliferating cell nuclear antigen gene | 1.0392687 | 1.0753464 | 1.0233837 | 1.0582508 | 1.0332854 | 1.440239 | 0.7330231 |
| Integrin beta1 | 1.0791886 | 1.2620445 | 1.6348858 | 1.7152888 | 1.4879615 | 2.1996462 | 0.9875876 |
| Caspase 6 | 1.0399905 | 1.1597272 | 1.2146997 | 1.3018749 | 1.2252991 | 1.327785 | 1.3398557 |
| Phase-1 RCT-116 | 1.9554826 | 2.1191454 | 1.851129 | 1.1381216 | 1.1273929 | 0.7562825 | 1.1538911 |
| Phase-1 RCT-109 | 0.8220774 | 1.1230259 | 1.0217818 | 0.9718144 | 1.090925 | 1.1919981 | 0.853956 |
| Phase-1 RCT-71 | 1.2169597 | 1.157805 | 1.2167754 | 1.2744727 | 1.4593581 | 1.1782391 | 0.650876 |
| Elongation factor-1 alpha | 0.7539177 | 1.0056561 | 0.944284 | 0.9122947 | 1.1391783 | 1.147543 | 1.3263549 |
| Phase-1 RCT-169 | 0.9855387 | 0.8199591 | 1.1758924 | 1.0246239 | 0.9600401 | 0.8769527 | 1.4113702 |
| Pyruvate kinase, muscle | 1.0239805 | 0.9824458 | 1.310553 | 1.1250604 | 1.2651156 | 1.438451 | 1.125992 |
| Nucleosome assembly protein | 1.1155065 | 1.3046411 | 0.5458274 | 0.6091545 | 0.820899 | 0.6249721 | 1.0993706 |
| Phase-1 RCT-127 | 1.1828607 | 1.1185426 | 1.6227692 | 1.1619661 | 1.3927568 | 1.600424 | 1.0434759 |
| Iron-responsive element-binding protein | 0.775624 | 0.8603781 | 0.8902442 | 0.9550924 | 1.0993336 | 0.8907909 | 0.9337031 |
| Phase-1 RCT-72 | 1.3251092 | 0.9330981 | 1.2209156 | 1.0102443 | 1.015847 | 0.9789583 | 1.1464983 |
| Phase-1 RCT-144 | 1.0295572 | 1.4272898 | 1.3024839 | 1.0295275 | 1.2864944 | 1.5411105 | 0.8180647 |
| Phase-1 RCT-242 | 1.2780957 | 1.0042795 | 1.5626116 | 1.1540312 | 1.0435572 | 1.2026746 | 0.8270812 |
| 17-beta hydroxysteroid dehydrogenase, | 1.6542811 | 2.0545876 | 0.310296 | 0.4121931 | 0.8468375 | 0.3620772 | 1.0858507 |
| type 2 | | | | | | | |
| Phase-1 RCT-70 | 1.1250782 | 1.0837983 | 1.16406 | 1.0629892 | 1.2148372 | 1.0299867 | 1.4420124 |
| Ribosomal protein L13A | 0.8047739 | 1.1515516 | 1.1293737 | 1.0434378 | 1.163287 | 1.173499 | 1.214916 |
| Cytochrome P450 2E1 | 0.9488558 | 0.8184056 | 0.6181762 | 0.9273856 | 0.9631865 | 0.7051388 | 0.6660162 |
| AT-3 | 1.1145523 | 0.9902331 | 1.0087068 | 1.0246154 | 0.8987333 | 1.068324 | 1.1539075 |
| Phase-1 RCT-270 | 0.7464272 | 0.7107219 | 0.7345905 | 0.7220982 | 0.7418329 | 0.6538084 | 1.1333648 |
| Phase-1 RCT-123 | 1.0694202 | 0.9182522 | 0.9286577 | 0.983676 | 0.8898334 | 0.9185084 | 0.7769177 |
| Alpha-2-macroglobulin, sequence 2 | 0.7243561 | 0.6533154 | 1.1841588 | 1.0675273 | 1.1136976 | 1.1515889 | 0.752406 |
| Matrix metalloproteinase-1 | 0.8492969 | 1.1798382 | 1.110358 | 0.8168539 | 1.1973447 | 1.0425521 | 0.9651782 |
| Beta-tubulin, class I | 1.0264555 | 1.8121955 | 1.1010879 | 0.9217299 | 1.1136973 | 1.0944594 | 0.6796358 |
| Phase-1 RCT-161 | 1.2073402 | 0.8442153 | 0.5472329 | 0.6981513 | 0.7327951 | 0.6350764 | 0.9153817 |
| 14-3-3 zeta | 1.5443193 | 1.8863462 | 0.8915179 | 0.9684742 | 1.0023837 | 1.1124685 | 0.9395816 |
| Stem cell factor | 0.966133 | 1.0268097 | 0.3445408 | 0.513709 | 0.5661957 | 0.2215127 | 0.5132113 |
| Macrophage inflammatory protein-2 alpha | 1.2013862 | 1.4419484 | 1.3694553 | 1.0956607 | 1.1506662 | 1.2285861 | 1.1066482 |
| Thymidylate synthase | 1.1121128 | 1.0964017 | 1.1336917 | 0.8667154 | 1.0024775 | 1.1142136 | 1.2201033 |
| c-jun | 1.4556346 | 1.5238326 | 1.6697975 | 1.3991966 | 1.2085371 | 1.2163249 | 1.0891458 |
| Superoxide dismutase Mn | 0.9487221 | 1.3575883 | 1.2195308 | 0.9977136 | 1.1495788 | 1.0753816 | 0.8297366 |
| Phase-1 RCT-73 | 0.9848855 | 1 | 0.7375651 | 0.8363152 | 0.8391286 | 0.8399628 | 1.197748 |
| Macrophage inflammatory protein-1 alpha | 1.2782801 | 0.9631984 | 1.3797879 | 1.5101252 | 1.141878 | 1.0564977 | 0.9866203 |
| Phase-1 RCT-214 | 1.0271447 | 1.205471 | 0.9266864 | 0.9358127 | 0.7303777 | 0.690338 | 1.0903677 |
| NADH-cytochrome b5 reductase | 0.7375527 | 0.8216204 | 0.9240202 | 0.8364075 | 1.0715731 | 0.7352684 | 0.8686571 |
| Interleukin-1 beta | 0.8359478 | 1.0747553 | 1.1811258 | 0.8399388 | 1.1410061 | 1.0731726 | 0.91253 |
| NADP-dependent isocitrate | 0.698113 | 0.9394422 | 0.6731242 | 0.8618017 | 0.8695845 | 0.7619873 | 1.0742916 |
| dehydrogenase, cytosolic | | | | | | | |
| Phase-1 RCT-289 | 0.7676363 | 0.7185834 | 0.7084163 | 0.9003491 | 0.8767054 | 0.9052573 | 0.6395382 |
| Glutamine synthetase | 0.5690295 | 0.9798326 | 0.6682612 | 0.7070361 | 0.868267 | 0.8789697 | 1.1746923 |
| Phase-1 RCT-182 | 0.9033766 | 0.7684806 | 0.8438702 | 0.8454312 | 0.7926207 | 0.876785 | 1.1865878 |
| Ubiquitin conjugating enzyme (RAD 6 | 1.0162641 | 1.0737911 | 1.1992924 | 1.3187292 | 1.0738155 | 1.2950537 | 0.80612 |
| homologue) | | | | | | | |
| Extracellular-signal-regulated kinase 1 | 0.8102462 | 0.7150808 | 0.652395 | 0.6043235 | 0.609538 | 0.18269 | 0.5922376 |
| Emerin | 1.0605819 | 1.1607376 | 0.8870008 | 1.0237916 | 1.0327363 | 1.0028857 | 1.191149 |
| Phase-1 RCT-78 | 0.9867514 | 1.0039544 | 0.7495351 | 0.9176893 | 0.8730167 | 0.9323676 | 0.9285804 |
| Phase-1 RCT-212 | 1.1195118 | 1.1763489 | 1.1240487 | 1.0815476 | 1.043643 | 0.9319655 | 0.7770124 |
| Phase-1 RCT-168 | 0.718448 | 0.9146082 | 0.6079588 | 0.7566543 | 0.723093 | 0.7969258 | 1.1398344 |
| Lysyl hydroxylase | 0.6534534 | 0.8343957 | 0.6563584 | 0.8276665 | 0.8891613 | 0.7564362 | 1.0925666 |
| Phase-1 RCT-55 | 0.9936612 | 0.9809362 | 1.6404183 | 1.1585383 | 1.0314525 | 0.7500727 | 0.8151295 |
| MHC class I antigen RT1.A1(f) alpha- | 1.4497671 | 1.8760674 | 1.4781014 | 1.2152092 | 1.2089397 | 1.0774136 | 1.0842497 |
| chain | | | | | | | |
| Phase-1 RCT-40 | 0.6459498 | 0.6521928 | 0.7296857 | 0.7067574 | 0.8118471 | 0.6822185 | 1.0952585 |
| Cyclin G | 2.065647 | 1.8833616 | 0.9291581 | 0.9702041 | 1.0424551 | 0.9836932 | 0.8775439 |
| Peroxisome proliferator activated receptor | 1.3342992 | 1.1436195 | 1.2041274 | 0.8344921 | 1.049409 | 0.9426634 | 1.3222736 |
| gamma | | | | | | | |
| Phase-1 RCT-280 | 1.1102597 | 1.0751228 | 0.7834436 | 1.1332078 | 0.8093881 | 0.8680679 | 1.0887243 |
| D-dopachrome tautomerase | 0.6867033 | 0.9294791 | 0.7719542 | 0.7489539 | 0.8316114 | 0.8137146 | 0.7590511 |
| Phase-1 RCT-287 | 0.8218606 | 0.9722557 | 0.7767792 | 0.9286861 | 1.0505296 | 0.9681942 | 1.1592729 |
| Phase-1 RCT-119 | 0.7977964 | 0.4444278 | 1.1775827 | 1.5250154 | 0.9430978 | 1.0476298 | 1.8847816 |
| Thioredoxin-1 (Trx1) | 0.7396792 | 0.9447732 | 1.1122036 | 0.8840528 | 1.0914489 | 1.1118892 | 0.8825707 |
| Phase-1 RCT-225 | 1.756609 | 1.5543699 | 1.3099439 | 1.3771476 | 1.2915497 | 1.0569124 | 0.7431795 |
| Protein kinase C alpha | 1.0435289 | 0.9538652 | 0.9195357 | 0.9333139 | 0.9760211 | 0.9726473 | 1.081073 |
| Phase-1 RCT-87 | 0.9529604 | 1.0964235 | 0.9861746 | 0.9904093 | 0.9733981 | 0.9292941 | 0.8236206 |
| Phase-1 RCT-59 | 1.4642415 | 1.1417861 | 1.5161198 | 1.3964022 | 1.3845748 | 1.3199365 | 1.0720131 |
| C4b-binding protein | 0.7506889 | 0.813801 | 1.4916167 | 0.6459799 | 0.6954152 | 0.7993727 | 1.1850541 |
| Phase-1 RCT-204 | 0.9917453 | 0.8797809 | 0.9322174 | 0.9458528 | 0.9514657 | 0.9374346 | 0.8564882 |
| Carnitine palmitoyl-CoA transferase | 1.8571333 | 2.1659138 | 1.1256486 | 1.3282723 | 1.1460063 | 1.118432 | 1.0043429 |
| 8-oxoguanine DNA glycosylase | 1.1141229 | 1.0104282 | 0.9755223 | 0.9688382 | 0.8869928 | 0.9963863 | 1.2477132 |
| H-rev107 | 0.9954786 | 0.8941421 | 0.9948268 | 0.9840605 | 0.8534088 | 0.9015765 | 0.9542361 |
| Preproalbumin | 1.1889983 | 1.3381602 | 0.3989481 | 1.1725336 | 0.9774386 | 0.9081649 | 1.3473572 |
| Phase-1 RCT-177 | 0.8614304 | 0.9857058 | 0.6785955 | 0.7430509 | 0.9552252 | 0.8574581 | 1.3310513 |
| Phase-1 RCT-110 | 1.0881591 | 0.9949583 | 0.9392828 | 1.0219705 | 0.8882209 | 0.9400039 | 0.8371259 |
| Phase-1 RCT-227 | 0.9843731 | 0.9979506 | 0.702757 | 0.9648183 | 0.9689232 | 0.9163446 | 0.9643975 |
| Neuropeptide Y | 1.6092019 | 0.8956645 | 0.9140371 | 0.9542439 | 0.9485049 | 1.0375732 | 0.9073355 |
| Phase-1 RCT-277 | 0.7800468 | 0.8577635 | 0.7392179 | 0.7106348 | 0.7846276 | 0.8019261 | 0.8010684 |
| Phase-1 RCT-229 | 1.1376607 | 0.9548941 | 0.9073708 | 1.0007855 | 0.9151645 | 0.9687908 | 0.9630218 |
| Phase-1 RCT-34 | 1.2971586 | 2.2257657 | 1.6183391 | 1.1257191 | 1.6556021 | 1.6096569 | 0.9728746 |
| Choline kinase | 1.0699079 | 1.0664937 | 1.2478535 | 1.0859207 | 1.2732768 | 1.1234984 | 0.5912794 |
| Phase-1 RCT-20 | 1.1795557 | 1.063569 | 1.1103457 | 1.1142442 | 0.99981 | 0.9877886 | 1.1781793 |
| Phase-1 RCT-248 | 0.9470415 | 1.113051 | 1.0963055 | 1.0056175 | 0.9694666 | 1.0463463 | 0.8796322 |
| | CHLOR | CHLOR | CHLOR | CHLOR | CHLOR | CIS | CIS |
| Compound-Dose (2) | 8 | 8 | 30 | 30 | 30 | 2.5 | 2.5 |
| Animal Number (3) | 42 | 43 | 51 | 52 | 53 | 321 | 322 |
| Liver Toxicity Necrosis Classification (4) | no | no | no | no | no | no | no |
| Gene Name (5) | | | | | | | |
| Gadd153 | 1.1154865 | 1.1477782 | 0.979353 | 0.9786103 | 1.3718704 | 1.0481455 | 1.5692155 |
| Interferon related developmental regulator | 1.2323107 | 1.2892762 | 1.4393816 | 1.2545093 | 1.3299127 | 0.8557413 | 0.9431457 |
| IFRD1 (PC4) | | | | | | | |
| ID-1 | 1.009092 | 1.1625088 | 0.9680634 | 1.0269444 | 1.2747144 | 1.2996411 | 0.9634217 |
| Insulin-like growth factor binding protein 1 | 0.8320085 | 1.3132583 | 1.9643286 | 1.6275401 | 2.8506594 | 2.8488114 | 1.5265641 |
| NIPK | 1.2523681 | 1.4276234 | 1.2224103 | 1.4314497 | 1.2301588 | 0.9326266 | 1.2489318 |
| Phase-1 RCT-50 | 1.0855274 | 0.9431125 | 1.0755401 | 1.1447283 | 1.1445996 | 1.1174988 | 1.667145 |
| Sarcoplasmic reticulum calcium ATPase | 0.8395885 | 0.7425658 | 0.9033074 | 1.0694333 | 0.7468906 | 0.8663188 | 1.0225002 |
| Phase-1 RCT-207 | 0.9661515 | 0.8826531 | 1.0364114 | 1.0625196 | 1.0972998 | 1.3269562 | 1.1799663 |
| Acetyl-CoA carboxylase | 1.2275119 | 1.251747 | 1.0627426 | 1.1022869 | 1.0763086 | 0.8155329 | 1.2310108 |
| Cathepsin L, sequence 2 | 1.0185919 | 1.1940762 | 1.4511142 | 1.3790704 | 2.1417127 | 0.8267224 | 1.137992 |
| Glucokinase | 0.7781501 | 0.6543509 | 0.6117292 | 0.7267367 | 0.7566555 | 0.9788308 | 0.8685898 |
| Gadd45 | 3.0108783 | 2.1356769 | 3.687135 | 4.3110094 | 5.290954 | 1.3367518 | 1.3935984 |
| Phase-1 RCT-18 | 1.188242 | 1.1966804 | 0.9794177 | 0.928596 | 0.9682339 | 0.7553352 | 0.9378159 |
| 3-beta-hydroxysteroid dehydrogenase | 1.1451674 | 1.3635805 | 0.9234186 | 1.0251063 | 1.0348244 | 0.7414269 | 0.5216857 |
| (HSD3B1) | | | | | | | |
| Phase-1 RCT-221 | 1.0733045 | 0.99972 | 0.8719192 | 0.8825889 | 0.9119783 | 1.0187305 | 0.9359502 |
| Heme oxygenase | 1.3874754 | 0.8787039 | 1.4452081 | 3.011199 | 1.6085197 | 1.4192125 | 3.0185418 |
| Cyclin dependent kinase 4 | 1.1646675 | 1.3098345 | 1.3348261 | 1.2825934 | 1.0658482 | 1.094088 | 1.168919 |
| Phase-1 RCT-75 | 1.1610053 | 1.0884984 | 1.059651 | 1.0045828 | 0.9392559 | 1.1481729 | 1.2807046 |
| Phase-1 RCT-65 | 1.1846508 | 0.9767454 | 0.8904515 | 0.8456168 | 1.0383489 | 1.1274593 | 1.0884659 |
| Cytochrome P450 1A1 | 0.8438214 | 0.8368002 | 1.1407958 | 1.1488457 | 1.069325 | 1.0374116 | 1.2092174 |
| Peroxisomal multifunctional enzyme type II | 0.9705853 | 1.0268742 | 1.0787603 | 0.9120428 | 1.0482578 | 1.1241759 | 1.024654 |
| DNA topoisomerase I | 1.6813105 | 1.8233294 | 1.0909226 | 1.3671016 | 1.0124493 | 1.3555889 | 1.2730944 |
| Focal adhesion kinase (pp125FAK) | 0.9386058 | 0.9418911 | 1.1970491 | 1.1677649 | 1.1902968 | 1.1562841 | 1.297927 |
| NGF-inducible anti-proliferative putative | 0.8700013 | 0.968384 | 0.7318949 | 0.9516211 | 0.7227963 | 1.2850078 | 1.8542887 |
| secreted protein (PC3) | | | | | | | |
| Phase-1 RCT-82 | 1.0077637 | 0.7940514 | 1.0103595 | 1.0150058 | 0.9358118 | 0.9103996 | 0.893941 |
| Senescence marker protein-30 | 0.6894271 | 0.544668 | 0.3751478 | 0.2975678 | 0.2899214 | 1.0017437 | 0.6969659 |
| Melanoma-associated antigen ME491 | 1.0673268 | 0.8962717 | 1.0344188 | 1.0140196 | 1.0908341 | 0.8575986 | 1.136686 |
| Activating transcription factor 3 | 1.2716261 | 1.5113707 | 1.5044177 | 1.6554366 | 1.2586535 | 1.0436833 | 1.0546737 |
| Tryptophan hydroxylase | 0.9248279 | 0.9845402 | 0.751968 | 1.0617217 | 0.8814269 | 1.1871979 | 0.8750411 |
| Zinc finger protein | 1.0814389 | 1.0481232 | 0.8425559 | 0.85352 | 0.853428 | 1.1352558 | 2.1485486 |
| c-myc | 1.0750235 | 1.0278102 | 1.5680552 | 2.2539406 | 1.5709214 | 1.0552783 | 1.1356846 |
| Cholesterol 7-alpha-hydroxylase (P450 | 0.9914135 | 0.8918626 | 1.040704 | 0.9511864 | 1.0649444 | 0.4833989 | 0.4078423 |
| VII) | | | | | | | |
| Ferritin H-chain | 0.9595605 | 1.13628 | 0.9677948 | 1.1391671 | 1.4343405 | 0.8794268 | 0.9496839 |
| Phase-1 RCT-197 | 1.2233564 | 1.2439715 | 1.4957703 | 1.261693 | 1.3718771 | 1.1491425 | 1.223004 |
| Phase-1 RCT-179 | 1.0486461 | 0.9897033 | 0.9421743 | 0.8767417 | 1.1014341 | 1.3035247 | 1.5393965 |
| Phase-1 RCT-15 | 1.3934708 | 1.66217 | 1.2167907 | 1.4751917 | 1.251944 | 2.2845128 | 2.2203672 |
| Argininosuccinate lyase | 1.3475709 | 1.3765392 | 1.18493 | 1.2778687 | 1.1076013 | 0.7997695 | 0.6047032 |
| Phase-1 RCT-49 | 1.1184026 | 0.9504643 | 1.084894 | 1.0051283 | 1.0952318 | 1.1647036 | 1.6599184 |
| Carbonic anhydrase III | 0.8121011 | 0.5098962 | 0.3730051 | 0.2512476 | 0.2467424 | 1.690513 | 0.2792007 |
| Proliferating cell nuclear antigen gene | 0.8010973 | 0.6425847 | 1.1095594 | 1.4573044 | 1.2800853 | 1.0867621 | 1.0850874 |
| Integrin beta1 | 0.8914644 | 1.652007 | 4.2395997 | 2.9845393 | 5.7773447 | 0.8929995 | 0.9970035 |
| Caspase 6 | 1.0795299 | 1.5765334 | 2.7078695 | 2.180241 | 2.897335 | 0.8565173 | 0.8945893 |
| Phase-1 RCT-116 | 0.9335246 | 0.9894401 | 1.1142278 | 1.0938298 | 1.3175815 | 1.135494 | 1.0426499 |
| Phase-1 RCT-109 | 0.9139678 | 1.0050579 | 0.9388721 | 1.3749807 | 1.1796324 | 0.9675121 | 1.0216907 |
| Phase-1 RCT-71 | 0.7249302 | 0.7228908 | 0.9070039 | 0.9619426 | 1.0990123 | 0.891835 | 0.8778606 |
| Elongation factor-1 alpha | 1.1669997 | 1.3890283 | 0.9479592 | 1.037878 | 1.2201952 | 1.0037752 | 1.0774795 |
| Phase-1 RCT-169 | 1.2170441 | 1.0586793 | 1.0043831 | 1.0007727 | 1.0552884 | 0.7101499 | 1.0227742 |
| Pyruvate kinase, muscle | 1.0220395 | 1.153401 | 1.4635667 | 1.3940432 | 2.3149333 | 0.7132008 | 0.8933905 |
| Nucleosome assembly protein | 1.8725564 | 1.2100004 | 1.4301475 | 0.9912605 | 0.8731886 | 0.729694 | 0.5878292 |
| Phase-1 RCT-127 | 0.91217 | 1.1790904 | 1.0916244 | 1.193346 | 1.3830885 | 1.2742819 | 1.6758145 |
| Iron-responsive element-binding protein | 0.9335291 | 1.0021856 | 1.1025468 | 1.0896357 | 0.9618915 | 0.8287777 | 0.7290085 |
| Phase-1 RCT-72 | 1.0443296 | 0.8009555 | 1.1727227 | 1.1287936 | 1.1589706 | 0.8790699 | 0.9629629 |
| Phase-1 RCT-144 | 0.9444373 | 0.9492301 | 1.0196669 | 1.0409471 | 1.0936794 | 1.4811122 | 1.760144 |
| Phase-1 RCT-242 | 0.8931469 | 0.7376085 | 0.9913535 | 1.0252755 | 1.0009165 | 1.5575988 | 2.30323 |
| 17-beta hydroxysteroid dehydrogenase, | 1.9170269 | 1.2345488 | 1.4456264 | 1.034977 | 0.9042218 | 0.2355756 | 0.1358948 |
| type 2 | | | | | | | |
| Phase-1 RCT-70 | 1.4256423 | 1.1970419 | 1.1723912 | 1.1445707 | 1.1097473 | 0.9919594 | 0.9467713 |
| Ribosomal protein L13A | 1.076386 | 1.3602656 | 1.1363965 | 1.3774645 | 1.7437706 | 1.0061979 | 1.0296408 |
| Cytochrome P450 2E1 | 0.6964331 | 0.4593764 | 0.5667268 | 0.5373628 | 0.8894759 | 1.1737031 | 0.8157266 |
| AT-3 | 1.0881056 | 1.2426351 | 1.0184362 | 0.9718655 | 1.088005 | 0.9846723 | 1.2640461 |
| Phase-1 RCT-270 | 1.1018115 | 1.0454168 | 0.8057656 | 0.6389422 | 0.7064616 | 0.7014405 | 0.4476357 |
| Phase-1 RCT-123 | 0.8459472 | 0.7076557 | 0.9484276 | 0.9002728 | 0.9271665 | 1.0168759 | 1.0553854 |
| Alpha-2-macroglobulin, sequence 2 | 0.8263649 | 0.7336202 | 0.7517352 | 0.8389691 | 0.7402446 | 1.1745716 | 1.2089303 |
| Matrix metalloproteinase-1 | 0.9857169 | 1.2091138 | 0.9980668 | 1.1738241 | 1.4422779 | 0.7997653 | 0.9389062 |
| Beta-tubulin, class I | 0.7394816 | 0.7929099 | 0.7704446 | 0.7849309 | 0.9101544 | 1.6775364 | 1.611548 |
| Phase-1 RCT-161 | 0.8937011 | 0.8659679 | 0.8909661 | 0.923851 | 0.8339379 | 0.9398942 | 0.6400846 |
| 14-3-3 zeta | 0.8022794 | 1.0374125 | 1.3065394 | 1.1916461 | 1.3609718 | 1.1379418 | 1.1328411 |
| Stem cell factor | 0.7100064 | 0.5766698 | 0.3998237 | 0.3254117 | 0.3033344 | 1.2216768 | 0.9465219 |
| Macrophage inflammatory protein-2 alpha | 1.2004474 | 1.122252 | 1.7133615 | 2.5194697 | 2.5560973 | 2.423721 | 3.6259134 |
| Thymidylate synthase | 0.8357665 | 0.938978 | 1.1650736 | 0.6426464 | 0.5549663 | 0.9653336 | 0.9737663 |
| c-jun | 0.9802616 | 0.9601364 | 1.7872092 | 1.83753 | 1.6349264 | 1.5117762 | 1.7973676 |
| Superoxide dismutase Mn | 0.782531 | 1.0818027 | 1.1022016 | 1.238344 | 2.2081156 | 1.9049118 | 3.100952 |
| Phase-1 RCT-73 | 1.3256644 | 1.2902198 | 1.152171 | 1.0634649 | 0.9158518 | 0.8070951 | 0.8796079 |
| Macrophage inflammatory protein-1 alpha | 0.9566952 | 0.9338902 | 0.9482654 | 0.9452609 | 1.0647439 | 1.0365449 | 1.1628245 |
| Phase-1 RCT-214 | 1.1875656 | 1.1366471 | 1.1611977 | 1.1408759 | 0.8960642 | 0.8748335 | 0.7626113 |
| NADH-cytochrome b5 reductase | 0.8898664 | 1.018042 | 0.8203328 | 0.7781595 | 0.9137035 | 0.8025321 | 0.6706873 |
| Interleukin-1 beta | 0.9383507 | 1.1372198 | 1.0331032 | 1.1905713 | 1.492238 | 1.1685706 | 1.3035988 |
| NADP-dependent isocitrate | 1.0505075 | 1.1625873 | 0.8700033 | 0.8569496 | 0.8815391 | 0.8715807 | 0.6579148 |
| dehydrogenase, cytosolic | | | | | | | |
| Phase-1 RCT-289 | 0.6273439 | 0.6261101 | 0.7069506 | 0.4988254 | 0.8197219 | 0.9840117 | 0.7083383 |
| Glutamine synthetase | 1.0122482 | 1.0133481 | 1.0876803 | 1.055616 | 1.156103 | 1.0742822 | 1.1382331 |
| Phase-1 RCT-182 | 1.0773256 | 0.7952755 | 0.6308931 | 0.5783318 | 0.6808135 | 0.9868669 | 0.6521811 |
| Ubiquitin conjugating enzyme (RAD 6 | 0.971682 | 0.830269 | 0.8264602 | 0.7788256 | 0.8922011 | 1.1171154 | 1.3759131 |
| homologue) | | | | | | | |
| Extracellular-signal-regulated kinase 1 | 0.840053 | 0.5107802 | 0.378788 | 0.2411319 | 0.2387902 | 1.0938401 | 0.9220062 |
| Emerin | 1.3089138 | 1.3182248 | 1.1625932 | 1.207086 | 0.9500165 | 0.9398523 | 1.1119928 |
| Phase-1 RCT-78 | 1.1101263 | 0.9190535 | 0.9802342 | 0.7581422 | 0.9202681 | 1 | 0.8794202 |
| Phase-1 RCT-212 | 0.6681351 | 0.6266831 | 0.717504 | 0.7221816 | 1.0439668 | 0.9715513 | 0.847584 |
| Phase-1 RCT-168 | 1.2306873 | 1.0887958 | 1.0488372 | 0.8321793 | 0.9547466 | 0.6862758 | 0.544033 |
| Lysyl hydroxylase | 0.9151855 | 1.0799072 | 1.0923643 | 1.0850555 | 1.1665833 | 0.6502548 | 1.1486886 |
| Phase-1 RCT-55 | 0.669755 | 0.7498632 | 0.7112371 | 0.7852803 | 1.2751788 | 0.8305684 | 0.7108417 |
| MHC class I antigen RT1.A1(f) alpha- | 0.9220004 | 0.9978906 | 1.0085784 | 1.1232988 | 1.1749231 | 1.8383648 | 1.3110204 |
| chain | | | | | | | |
| Phase-1 RCT-40 | 1.142313 | 1.1614307 | 0.8589244 | 0.8829776 | 0.89521 | 0.9825496 | 0.9357773 |
| Cyclin G | 0.818247 | 1.2084734 | 1.2109188 | 1.2326605 | 1.9763862 | 2.088708 | 2.5875628 |
| Peroxisome proliferator activated receptor | 0.8761163 | 1.010359 | 1.1754947 | 0.6825765 | 0.537784 | 0.8231603 | 0.8158611 |
| gamma | | | | | | | |
| Phase-1 RCT-280 | 1.1900065 | 1.163371 | 1.1028711 | 1.1484097 | 0.8714736 | 1.0523067 | 1.0601717 |
| D-dopachrome tautomerase | 0.7296417 | 0.7144721 | 0.6082565 | 0.5594831 | 0.6428797 | 0.9757455 | 0.7716169 |
| Phase-1 RCT-287 | 1.1467422 | 1.2848513 | 0.985082 | 0.9427435 | 1.0710658 | 1.2319392 | 0.9286174 |
| Phase-1 RCT-119 | 1.9680895 | 1.5405396 | 1.1503268 | 0.8815578 | 1.0061892 | 0.9181774 | 0.4936768 |
| Thioredoxin-1 (Trx1) | 0.8554814 | 0.9188081 | 0.9507105 | 1.0061847 | 1.1137342 | 1.0669836 | 1.0990372 |
| Phase-1 RCT-225 | 0.6378572 | 0.6402514 | 0.6947967 | 0.6845623 | 1.1247225 | 1.2019472 | 1.7485437 |
| Protein kinase C alpha | 1.143705 | 1.3090938 | 1.1024612 | 1.1266302 | 1.275723 | 0.8042295 | 0.9529098 |
| Phase-1 RCT-87 | 0.881456 | 0.769196 | 0.9429412 | 0.874217 | 0.9524851 | 1.0375906 | 1.036284 |
| Phase-1 RCT-59 | 1.0565059 | 0.9623858 | 1.0298393 | 1.0481942 | 1.1524539 | 1.5674758 | 1.4307327 |
| C4b-binding protein | 1.1664581 | 1.233799 | 0.8938183 | 0.8018568 | 0.933904 | 0.8525462 | 0.8196399 |
| Phase-1 RCT-204 | 0.925979 | 0.8856897 | 0.8716339 | 0.8313951 | 0.9047712 | 1.0613654 | 1.0805084 |
| Carnitine palmitoyl-CoA transferase | 0.8696795 | 1.2100221 | 2.0888746 | 2.074062 | 1.2719198 | 1.2743616 | 1.2639215 |
| 8-oxoguanine DNA glycosylase | 1.2628317 | 1.3998804 | 1.2313519 | 1.1952983 | 1.3921142 | 1.0041099 | 1.0533049 |
| H-rev107 | 0.9139517 | 1.0500882 | 0.9810633 | 1.0832365 | 1.1479695 | 0.9965332 | 1.0774955 |
| Preproalbumin | 1.3677338 | 1.6960224 | 1.3043967 | 1.6239588 | 1.2593548 | 0.8818493 | 0.7395453 |
| Phase-1 RCT-177 | 1.5262921 | 1.3890699 | 1.126783 | 1.0145448 | 0.9118512 | 0.914107 | 0.8568504 |
| Phase-1 RCT-110 | 0.9419355 | 0.8569527 | 0.7316012 | 0.7485083 | 0.7525227 | 0.9903163 | 1.0568191 |
| Phase-1 RCT-227 | 1.1219705 | 0.9706979 | 1.0148945 | 0.7522841 | 0.9777166 | 1.1418369 | 0.7081797 |
| Neuropeptide Y | 0.9518693 | 0.8858903 | 0.820267 | 0.7629712 | 0.8320915 | 0.9568901 | 0.8320125 |
| Phase-1 RCT-277 | 0.6252193 | 0.9207644 | 0.6557452 | 0.9625894 | 1.0395354 | 0.8916547 | 1.0027869 |
| Phase-1 RCT-229 | 1.109886 | 0.8607388 | 0.9948127 | 0.7848485 | 0.9082511 | 1.0212615 | 0.7928672 |
| Phase-1 RCT-34 | 0.9637625 | 0.9899156 | 0.9474135 | 0.894336 | 0.9985528 | 0.5605913 | 0.4376034 |
| Choline kinase | 0.8542138 | 0.7978244 | 0.9789072 | 1.1913267 | 0.7598936 | 0.9692103 | 1.1535779 |
| Phase-1 RCT-20 | 1.1604072 | 1.1660671 | 1.1193624 | 1.2247064 | 1.2229095 | 0.9824191 | 1.1543435 |
| Phase-1 RCT-248 | 0.9401617 | 0.9771152 | 1.0882425 | 1.1208252 | 1.2611833 | 1.0943983 | 1.0652899 |
| | CIS | CIS | CIS | CIS | CLO | CLO | CLO |
| Compound-Dose (2) | 2.5 | 10 | 10 | 10 | 75 | 75 | 75 |
| Animal Number (3) | 323 | 331 | 332 | 333 | 321 | 322 | 323 |
| Liver Toxicity Necrosis Classification (4) | no | no | no | no | no | no | no |
| Gene Name (5) | | | | | | | |
| Gadd153 | 1.4777994 | 1.1664515 | 1.3535161 | 1.3058107 | 1.0652044 | 0.8028036 | 0.9492043 |
| Interferon related developmental regulator | 1.1955168 | 0.9615454 | 1.1182783 | 1.1427512 | 0.9723983 | 1.4994612 | 0.971494 |
| IFRD1 (PC4) | | | | | | | |
| ID-1 | 1.160696 | 0.8342257 | 0.891918 | 1.0084596 | 1.039946 | 0.9025534 | 1.330385 |
| Insulin-like growth factor binding protein 1 | 0.9627976 | 2.0935564 | 1.2717538 | 1.4697685 | 1.635953 | 1.6682786 | 1.6886628 |
| NIPK | 0.9714177 | 0.9794728 | 0.8894091 | 1.0045447 | 0.9442859 | 0.9880772 | 1.2306856 |
| Phase-1 RCT-50 | 1.2421951 | 1.6245226 | 1.4912567 | 1.6132729 | 1.0215045 | 0.8098882 | 1.0130222 |
| Sarcoplasmic reticulum calcium ATPase | 1.0362289 | 1.0038043 | 1.0730094 | 0.9356644 | 0.9362705 | 1.2452916 | 1.0652164 |
| Phase-1 RCT-207 | 1.1396152 | 2.3001273 | 1.2054566 | 2.2146907 | 0.9938449 | 0.9965997 | 1.3133621 |
| Acetyl-CoA carboxylase | 1.6246902 | 1.0086026 | 1.0018318 | 0.8330166 | 0.9443953 | 1.1103008 | 0.8124099 |
| Cathepsin L, sequence 2 | 1.2705058 | 1.4486492 | 1.6149896 | 1.8223121 | 0.9425534 | 1.1856511 | 1.0741558 |
| Glucokinase | 1.9152843 | 0.8090051 | 0.7899045 | 0.6177193 | 0.8080473 | 1.175971 | 1.5373787 |
| Gadd45 | 0.9053156 | 2.0765855 | 2.1534743 | 1.7632583 | 1.4377085 | 0.7706204 | 1.1678901 |
| Phase-1 RCT-18 | 0.9070922 | 0.9649357 | 0.9006067 | 0.8840186 | 0.9361278 | 0.8816857 | 0.9858773 |
| 3-beta-hydroxysteroid dehydrogenase | 0.7652606 | 0.7189758 | 0.7084202 | 1.0780706 | 0.8785093 | 1.0495851 | 0.9912571 |
| (HSD3B1) | | | | | | | |
| Phase-1 RCT-221 | 1.0829856 | 0.9066542 | 0.9394296 | 1.0299114 | 1.0559938 | 1.2270768 | 0.9668486 |
| Heme oxygenase | 1.3564284 | 1.6648824 | 2.280217 | 1.8524593 | 0.8626621 | 1.1159544 | 0.6762078 |
| Cyclin dependent kinase 4 | 1.2025174 | 0.9642014 | 0.9589586 | 0.9617328 | 1.059544 | 1.096654 | 1.0562112 |
| Phase-1 RCT-75 | 1.5780407 | 1.3445679 | 1.1163859 | 1.3550801 | 1.0085129 | 0.8116758 | 0.9229184 |
| Phase-1 RCT-65 | 0.9324543 | 1.0161432 | 0.9659664 | 0.9840442 | 1.0599922 | 0.8264915 | 0.9000595 |
| Cytochrome P450 1A1 | 0.7781223 | 1.0082821 | 1.069517 | 0.6654941 | 0.8941606 | 0.6997943 | 0.9479065 |
| Peroxisomal multifunctional enzyme type II | 1.2428923 | 0.9375581 | 1.015255 | 1.3469954 | 1.535029 | 1.4449558 | 1.1875936 |
| DNA topoisomerase I | 1.3691139 | 1.067189 | 0.9850302 | 1.1193889 | 1.0893594 | 1.4879628 | 1.1045711 |
| Focal adhesion kinase (pp125FAK) | 1.0777004 | 1.001217 | 1.0490468 | 0.9466199 | 0.9603059 | 1.1272535 | 1.0019656 |
| NGF-inducible anti-proliferative putative | 1.2825193 | 3.7288678 | 4.6822386 | 5.0091543 | 1.108902 | 1.467495 | 1.0197836 |
| secreted protein (PC3) | | | | | | | |
| Phase-1 RCT-82 | 0.9664533 | 0.939314 | 1.0486693 | 0.9060658 | 0.9461124 | 1.0901719 | 1.0111241 |
| Senescence marker protein-30 | 0.9826746 | 0.4342715 | 0.4231782 | 1.0299826 | 0.7542052 | 0.983812 | 0.9253538 |
| Melanoma-associated antigen ME491 | 1.1213249 | 1.1245394 | 1.2892827 | 1.574742 | 0.9445653 | 1.0266773 | 0.9903634 |
| Activating transcription factor 3 | 0.8149502 | 1.0794685 | 0.9563219 | 2.1110792 | 1.2880273 | 0.7614387 | 1.1390245 |
| Tryptophan hydroxylase | 1.1159321 | 0.9139798 | 1.1211963 | 1.0405108 | 0.9023335 | 0.9849532 | 1 |
| Zinc finger protein | 1.1880169 | 3.0201125 | 1.2276562 | 3.382819 | 0.8863083 | 0.9204525 | 0.9406682 |
| c-myc | 0.8760836 | 1.3631748 | 1.3588105 | 1.2821947 | 1.4059875 | 0.7448381 | 1.078347 |
| Cholesterol 7-alpha-hydroxylase (P450 | 0.4432934 | 0.5973987 | 0.4257 | 0.2798733 | 0.7894118 | 1.0356333 | 1.1176172 |
| VII) | | | | | | | |
| Ferritin H-chain | 1.2013538 | 1.1310893 | 1.0276756 | 1.2717515 | 0.8541849 | 0.8118534 | 0.7966287 |
| Phase-1 RCT-197 | 1.2207997 | 1.7883906 | 2.8310745 | 2.6738167 | 0.9620302 | 1.1364459 | 0.8503624 |
| Phase-1 RCT-179 | 1.5649978 | 1.8167458 | 1.37526 | 1.7958474 | 1.1558181 | 1.4054419 | 1.1541637 |
| Phase-1 RCT-15 | 1.6124748 | 2.5848107 | 1.963651 | 1.8955631 | 1.1780736 | 0.8423352 | 1.0149719 |
| Argininosuccinate lyase | 0.6987714 | 0.8085209 | 0.8078077 | 0.7143409 | 1.0030717 | 0.4414504 | 1.2496835 |
| Phase-1 RCT-49 | 1.287144 | 1.7381076 | 2.1042411 | 2.2800722 | 0.9679553 | 1.3272383 | 1.2781005 |
| Carbonic anhydrase III | 1.3774656 | 0.4829224 | 0.6465321 | 0.5195742 | 0.8177142 | 0.4862387 | 0.5968595 |
| Proliferating cell nuclear antigen gene | 1.0372772 | 1.3832303 | 1.2763162 | 1.033595 | 0.9915773 | 1.1778843 | 1.1405118 |
| Integrin beta1 | 1.0962051 | 0.9572582 | 1.0701771 | 1.0992563 | 1.2558843 | 1.1401267 | 1.2200412 |
| Caspase 6 | 1.0105057 | 0.8008647 | 1.0239257 | 0.9371974 | 0.8797274 | 0.8247346 | 1.0478537 |
| Phase-1 RCT-116 | 0.8992659 | 0.8865593 | 1.1610253 | 0.7470042 | 1.1990987 | 0.6092429 | 0.9772598 |
| Phase-1 RCT-109 | 1.0670938 | 1.0749619 | 0.9599282 | 1.1057761 | 1.5693303 | 1.8476527 | 1.6787325 |
| Phase-1 RCT-71 | 0.9874322 | 0.8550908 | 0.9017765 | 0.927092 | 0.9488192 | 1.0501238 | 1.0651696 |
| Elongation factor-1 alpha | 1.1615913 | 1.0136976 | 1.0486158 | 1.2029771 | 1.0200361 | 1.463058 | 0.9113765 |
| Phase-1 RCT-169 | 0.8380023 | 0.6616225 | 0.913646 | 1.1546594 | 0.940307 | 0.8193247 | 0.9426071 |
| Pyruvate kinase, muscle | 0.671881 | 1.1697266 | 1.0343255 | 0.6498295 | 1.1389663 | 0.8554031 | 0.9773169 |
| Nucleosome assembly protein | 0.7201088 | 0.6301301 | 0.6103741 | 0.7249172 | 1.2080947 | 0.9989293 | 0.8471467 |
| Phase-1 RCT-127 | 1.3289052 | 1.443341 | 0.9583329 | 1.4266738 | 0.7991654 | 1.2295078 | 1.1102518 |
| Iron-responsive element-binding protein | 0.9552711 | 0.6928353 | 0.9271944 | 0.9041993 | 1.3774465 | 1.3186419 | 1.2631097 |
| Phase-1 RCT-72 | 1.0580403 | 0.8638771 | 1.0756698 | 1.1189767 | 0.9157062 | 0.8688906 | 0.9227018 |
| Phase-1 RCT-144 | 1.4925249 | 1.682965 | 1.859191 | 2.1793988 | 1.2780149 | 1.506084 | 1.1756432 |
| Phase-1 RCT-242 | 0.974168 | 3.7794254 | 6.070761 | 4.648406 | 0.9758751 | 0.7302912 | 0.9303653 |
| 17-beta hydroxysteroid dehydrogenase, | 0.1311868 | 0.1300119 | 0.1778954 | 0.0978842 | 1.450668 | 1.0072777 | 0.9387043 |
| type 2 | | | | | | | |
| Phase-1 RCT-70 | 0.918063 | 0.9006651 | 1.0513812 | 1.0233526 | 1.100938 | 1.0677193 | 1.2297233 |
| Ribosomal protein L13A | 1.0786726 | 1.1738036 | 0.9829432 | 1.1535915 | 1.7683101 | 1.7414174 | 1.6344805 |
| Cytochrome P450 2E1 | 0.9180553 | 0.7208829 | 0.6410477 | 0.6841291 | 0.6991042 | 0.5652058 | 1.0769557 |
| AT-3 | 0.9036488 | 1.1403708 | 1.3955013 | 0.8208142 | 0.9548324 | 1.061699 | 1.0325601 |
| Phase-1 RCT-270 | 0.8484456 | 0.5616964 | 0.5611181 | 0.6147419 | 0.5885986 | 0.7882578 | 0.6843891 |
| Phase-1 RCT-123 | 0.9339474 | 0.9256986 | 1.0203613 | 0.8938387 | 0.9630592 | 0.8294137 | 0.9803798 |
| Alpha-2-macroglobulin, sequence 2 | 1.0340519 | 1.2915121 | 1.4530298 | 0.9990516 | 0.7747713 | 1.0868514 | 0.9665895 |
| Matrix metalloproteinase-1 | 1.0210088 | 1.1975784 | 1.1657771 | 1.1477946 | 0.915849 | 1.0194211 | 0.9277153 |
| Beta-tubulin, class I | 2.1502948 | 1.8103998 | 1.301003 | 1.8635764 | 1.1354531 | 0.5927039 | 0.8893524 |
| Phase-1 RCT-161 | 0.9292438 | 0.9322262 | 0.9465597 | 0.7725709 | 0.8508648 | 0.5625117 | 0.6329725 |
| 14-3-3 zeta | 1.2869604 | 1.2892867 | 1.2460713 | 1.5517802 | 1.2941656 | 0.8575548 | 1.1797345 |
| Stem cell factor | 0.9512779 | 0.7574608 | 0.9154466 | 0.8386632 | 0.8919016 | 0.8942381 | 0.9643994 |
| Macrophage inflammatory protein-2 alpha | 1.5741637 | 6.610779 | 5.254996 | 2.702853 | 1.3815869 | 0.7928991 | 0.7332662 |
| Thymidylate synthase | 0.9324723 | 1.8106767 | 1.848705 | 1.143121 | 1.0171559 | 0.8190426 | 0.9500609 |
| c-jun | 0.8263295 | 1.4880482 | 1.859949 | 1.1067829 | 1.2352223 | 0.8755413 | 0.985602 |
| Superoxide dismutase Mn | 2.1785557 | 3.1054919 | 2.5423782 | 2.5245397 | 1.3782283 | 1.7269521 | 1.3459438 |
| Phase-1 RCT-73 | 0.8896431 | 0.8799521 | 0.8488047 | 0.9163806 | 1.1911749 | 1.0821598 | 1.1916877 |
| Macrophage inflammatory protein-1 alpha | 0.8031789 | 1.5727121 | 0.7559398 | 1.2641194 | 1.0470961 | 0.6532739 | 0.9808293 |
| Phase-1 RCT-214 | 0.9874226 | 0.7348059 | 0.5833508 | 0.8406636 | 1.0219152 | 0.8823368 | 0.6966664 |
| NADH-cytochrome b5 reductase | 0.9815897 | 0.6495572 | 0.6188718 | 0.7931231 | 0.7872851 | 0.7973765 | 0.7505189 |
| Interleukin-1 beta | 1.3067657 | 1.4386387 | 17.1902502 | 1.4913352 | 0.9446659 | 1.0470198 | 0.9997647 |
| NADP-dependent isocitrate | 0.9430142 | 0.5803416 | 0.6742474 | 0.7954767 | 0.9640394 | 0.9352261 | 0.8890194 |
| dehydrogenase, cytosolic | | | | | | | |
| Phase-1 RCT-289 | 0.9783509 | 0.7672762 | 0.7188644 | 0.9467017 | 0.9060549 | 1.1170591 | 0.991654 |
| Glutamine synthetase | 1.4118668 | 1.0244579 | 0.9458515 | 1.1529943 | 0.9984097 | 1.1699831 | 0.9126689 |
| Phase-1 RCT-182 | 1.0938393 | 0.719855 | 1.2789668 | 0.8368481 | 0.6377794 | 0.8835357 | 0.7953116 |
| Ubiquitin conjugating enzyme (RAD 6 | 1.3615159 | 1.344485 | 1.2133102 | 1.3712791 | 1.0965292 | 1.4055922 | 1.1337833 |
| homologue) | | | | | | | |
| Extracellular-signal-regulated kinase 1 | 0.8834366 | 0.9233549 | 0.8853308 | 0.7421242 | 0.9318174 | 0.55033 | 0.7326925 |
| Emerin | 0.9929712 | 0.979807 | 1.1339457 | 0.8230648 | 1.3849081 | 1.4212873 | 1.7454102 |
| Phase-1 RCT-78 | 1.0631474 | 0.8703955 | 1.0930982 | 1 | 0.7915409 | 0.89054 | 0.7870286 |
| Phase-1 RCT-212 | 1.0408399 | 1.0012947 | 1.0971035 | 1.0367546 | 0.8877345 | 0.902493 | 0.8932788 |
| Phase-1 RCT-168 | 0.8991906 | 0.5307823 | 0.5121778 | 0.4479933 | 1.0402095 | 1.3195096 | 1.0798941 |
| Lysyl hydroxylase | 1.2557335 | 1.1076778 | 1.0425076 | 1.4199129 | 1.0123721 | 1.2128575 | 0.9269668 |
| Phase-1 RCT-55 | 0.9069951 | 0.6808924 | 0.8638609 | 0.8238348 | 0.9664424 | 0.973132 | 0.8962677 |
| MHC class I antigen RT1.A1(f) alpha- | 1.1089827 | 1.3744236 | 1.6525911 | 1.2688493 | 1.0875078 | 0.7548979 | 0.9652919 |
| chain | | | | | | | |
| Phase-1 RCT-40 | 1.333961 | 0.8317008 | 0.7415124 | 0.9939089 | 0.6926498 | 0.9241318 | 0.6660984 |
| Cyclin G | 2.8529127 | 6.4126663 | 5.5224924 | 5.7263427 | 1.2905228 | 0.8943251 | 0.9488641 |
| Peroxisome proliferator activated receptor | 0.7424828 | 0.7344958 | 0.6858305 | 0.5856412 | 1.3696257 | 1.1071922 | 0.8844312 |
| gamma | | | | | | | |
| Phase-1 RCT-280 | 0.8274566 | 1.1065544 | 0.6408547 | 0.7929913 | 0.9980474 | 1.0670675 | 1.0039196 |
| D-dopachrome tautomerase | 1.3433974 | 0.9433621 | 0.8984301 | 1.0952792 | 0.8260452 | 0.9878911 | 0.7970778 |
| Phase-1 RCT-287 | 1.0541203 | 0.918619 | 1.1302222 | 1.095956 | 1.1517566 | 1.2045815 | 1.0615433 |
| Phase-1 RCT-119 | 0.8560834 | 0.6704112 | 0.6299486 | 0.6250264 | 0.5576165 | 0.8699736 | 1.082693 |
| Thioredoxin-1 (Trx1) | 1.3200029 | 1.2004397 | 1.3720669 | 1.5935584 | 1.148264 | 1.5803232 | 1.0829506 |
| Phase-1 RCT-225 | 1.1241944 | 0.6642692 | 1.0253955 | 1.0692991 | 1.1783477 | 1.2785318 | 1.0276109 |
| Protein kinase C alpha | 0.8914604 | 1.022812 | 1.0834047 | 0.8634725 | 0.939705 | 1.2789294 | 0.9421706 |
| Phase-1 RCT-87 | 1.1108028 | 1.0543126 | 1.1116376 | 1.277041 | 0.869207 | 1.0637668 | 0.8390908 |
| Phase-1 RCT-59 | 1.0592649 | 3.1518254 | 2.462888 | 3.713464 | 1.104827 | 0.850275 | 1.0224192 |
| C4b-binding protein | 0.7784235 | 0.8231621 | 1.072345 | 0.7397378 | 0.9645513 | 1.2008616 | 0.8601201 |
| Phase-1 RCT-204 | 0.9647512 | 0.903746 | 1.0038626 | 0.9705741 | 0.9136388 | 0.8995572 | 1.018074 |
| Carnitine palmitoyl-CoA transferase | 0.7535704 | 1.0858845 | 1.1620414 | 1.0611956 | 1.2688335 | 0.8885603 | 1.4870517 |
| 8-oxoguanine DNA glycosylase | 1.0239844 | 0.9746443 | 1.1159245 | 0.796319 | 1.1077858 | 0.9777014 | 1.0878283 |
| H-rev107 | 0.9135078 | 0.8652567 | 0.6880069 | 0.8309463 | 0.8682193 | 0.507876 | 0.9559618 |
| Preproalbumin | 0.9987862 | 0.8239122 | 0.9337972 | 1.1313337 | 1.0173795 | 0.5518554 | 0.738997 |
| Phase-1 RCT-177 | 1.0703257 | 0.7481232 | 0.7475388 | 1.0941818 | 1.127677 | 1.1088003 | 1.1092864 |
| Phase-1 RCT-110 | 0.9773096 | 1.0059986 | 1.0569994 | 0.8530992 | 0.9480823 | 0.7700564 | 0.8653486 |
| Phase-1 RCT-227 | 1.0658389 | 0.8278618 | 1.1731341 | 1.4920981 | 0.7604884 | 0.9995962 | 0.7956342 |
| Neuropeptide Y | 0.9245049 | 1.2350802 | 1.2737019 | 0.8446038 | 0.9248889 | 0.8652428 | 0.9337464 |
| Phase-1 RCT-277 | 1.0167917 | 0.9299302 | 0.7974231 | 0.9603907 | 0.8900505 | 0.9842934 | 0.8678485 |
| Phase-1 RCT-229 | 0.980537 | 1.0672075 | 1.6240153 | 0.8684574 | 0.8950664 | 0.7411576 | 0.8942618 |
| Phase-1 RCT-34 | 0.6976548 | 0.6147797 | 0.8247668 | 0.6483958 | 0.9870291 | 0.9411096 | 0.9181494 |
| Choline kinase | 1.1147641 | 1.0482554 | 1.228698 | 1.1860961 | 0.9611791 | 0.8724414 | 1.1537596 |
| Phase-1 RCT-20 | 1.0598736 | 1.1521776 | 1.1222556 | 1.0722178 | 1.0241532 | 0.6953397 | 0.8795231 |
| Phase-1 RCT-248 | 0.8798857 | 0.8580287 | 1.0154983 | 0.9643522 | 1.0723004 | 1.251142 | 1.3533441 |
| | CLO | CLO | CLO | CLOZ | CLOZ | CLOZ | CLOZ |
| Compound-Dose (2) | 250 | 250 | 250 | 45 | 45 | 45 | 180 |
| Animal Number (3) | 1841 | 1842 | 1843 | 2421 | 2422 | 2423 | 2431 |
| Liver Toxicity Necrosis Classification (4) | no | no | no | no | no | no | no |
| Gene Name (5) | | | | | | | |
| Gadd153 | 0.9608976 | 1.1410654 | 0.9374231 | 1.1182563 | 0.9448752 | 1.0050619 | 0.9333098 |
| Interferon related developmental regulator | 0.8810267 | 0.7182581 | 0.7748178 | 1.0875887 | 1.2180828 | 0.937407 | 0.9268757 |
| IFRD1 (PC4) | | | | | | | |
| ID-1 | 1.0680745 | 1.2008054 | 1.1652457 | 0.7702386 | 0.8847795 | 0.9727145 | 1.0591007 |
| Insulin-like growth factor binding protein 1 | 1.1164829 | 1.5894657 | 1.1699874 | 1.4766254 | 1.07835 | 1.3163824 | 1.0187067 |
| NIPK | 0.8366485 | 0.8146783 | 0.8091981 | 1.0655283 | 1.2932701 | 1.000479 | 1.1249355 |
| Phase-1 RCT-50 | 0.9042951 | 1.0352192 | 0.9889638 | 0.918011 | 1.027393 | 0.9101507 | 0.9771627 |
| Sarcoplasmic reticulum calcium ATPase | 0.9992048 | 1.0909557 | 0.9665774 | 1.1402038 | 1.1398048 | 1.0184636 | 0.98317 |
| Phase-1 RCT-207 | 1.0316937 | 1.5294598 | 1.3800668 | 0.9013122 | 0.9000346 | 0.9108143 | 0.9625818 |
| Acetyl-CoA carboxylase | 0.9077064 | 0.943137 | 0.865503 | 1.1002442 | 1.5209866 | 0.8949486 | 1.0765935 |
| Cathepsin L, sequence 2 | 1.0163392 | 1.1988395 | 1.0473108 | 1.0775472 | 1.0766658 | 1.0270677 | 0.9306462 |
| Glucokinase | 0.8913963 | 1.2220505 | 0.9930557 | 0.7689819 | 0.6701633 | 0.4975296 | 0.9185473 |
| Gadd45 | 1.4709898 | 1.0246946 | 0.9917869 | 1.1511537 | 1.0001892 | 0.8837206 | 0.9226152 |
| Phase-1 RCT-18 | 0.9673203 | 0.9994537 | 0.942918 | 0.8590237 | 0.9421533 | 0.8213382 | 0.8824967 |
| 3-beta-hydroxysteroid dehydrogenase | 1.0359262 | 0.8313455 | 1.0069315 | 0.7170981 | 0.9940825 | 1.1695011 | 1.0599293 |
| (HSD3B1) | | | | | | | |
| Phase-1 RCT-221 | 1.159901 | 0.9301622 | 0.9389841 | 0.9978771 | 1.0921589 | 0.9513888 | 0.9869565 |
| Heme oxygenase | 0.8119684 | 0.5829337 | 0.6484602 | 0.9824582 | 1.6913332 | 1.0439022 | 1.6500137 |
| Cyclin dependent kinase 4 | 1.0012888 | 0.995525 | 1.093948 | 1.0530684 | 0.8964134 | 0.9068167 | 0.9177654 |
| Phase-1 RCT-75 | 0.9956465 | 0.8213385 | 0.7747017 | 1.00311 | 1.1575559 | 0.9998019 | 1.0270292 |
| Phase-1 RCT-65 | 0.9948198 | 1.150421 | 0.96705 | 1.3164803 | 1.1347224 | 1.0892775 | 1.2174214 |
| Cytochrome P450 1A1 | 0.9418458 | 1.7376593 | 0.9267996 | 0.7708598 | 0.8532201 | 0.7834163 | 5.138333 |
| Peroxisomal multifunctional enzyme type II | 1.485648 | 1.1685367 | 1.2926756 | 1.2339665 | 1.3282776 | 1.2846198 | 1.1289783 |
| DNA topoisomerase I | 0.8436759 | 0.5904681 | 0.733056 | 1.3459347 | 1.3590252 | 1.2516785 | 1.0840245 |
| Focal adhesion kinase (pp125FAK) | 0.9672682 | 0.8894373 | 0.8900354 | 0.9147795 | 0.9625965 | 0.9351348 | 0.8391174 |
| NGF-inducible anti-proliferative putative | 0.9825646 | 0.7283962 | 0.9459561 | 1.048002 | 1.1530355 | 0.9120295 | 1.0493531 |
| secreted protein (PC3) | | | | | | | |
| Phase-1 RCT-82 | 0.9453846 | 0.968136 | 1.0821024 | 0.9561508 | 0.9802937 | 0.8246241 | 0.9458508 |
| Senescence marker protein-30 | 0.4211526 | 0.4748625 | 1.1090428 | 1.4753989 | 1.0794756 | 0.8026514 | 0.9307346 |
| Melanoma-associated antigen ME491 | 1.0222632 | 1.4581567 | 1.0210073 | 0.7708532 | 0.8438385 | 1.064818 | 0.822859 |
| Activating transcription factor 3 | 0.4920422 | 1.2822056 | 1.1339073 | 1.1734228 | 1.0272698 | 1.2657508 | 0.9528345 |
| Tryptophan hydroxylase | 1.0754423 | 0.7199513 | 0.9398391 | 1.336392 | 1.0951045 | 1.340614 | 0.9612781 |
| Zinc finger protein | 1.035419 | 0.9452173 | 0.9199344 | 0.7687463 | 0.8495618 | 0.8543456 | 0.9688488 |
| c-myc | 1.1188986 | 1.0494416 | 0.9548339 | 0.7265537 | 0.9567418 | 0.7177096 | 0.9443395 |
| Cholesterol 7-alpha-hydroxylase (P450 | 0.9309568 | 2.6260757 | 1.0142232 | 1.4397609 | 0.9356353 | 1.0783836 | 0.9886511 |
| VII) | | | | | | | |
| Ferritin H-chain | 1.0074964 | 0.749508 | 1.0745804 | 1.0580496 | 0.9748812 | 1.0133928 | 0.9146249 |
| Phase-1 RCT-197 | 0.9017187 | 1.0155311 | 0.9051011 | 1.0756719 | 0.8391094 | 0.9836285 | 0.8669242 |
| Phase-1 RCT-179 | 1.1299447 | 0.9664941 | 1.0256494 | 1.0455304 | 1.0676757 | 1.0275779 | 1.1828905 |
| Phase-1 RCT-15 | 1.0055814 | 0.9688541 | 0.9799063 | 1.3965299 | 1.7235432 | 1.1343454 | 1.2318407 |
| Argininosuccinate lyase | 1.2846969 | 1.495419 | 0.9447678 | 1.422001 | 1.1525767 | 1.3732553 | 1.245413 |
| Phase-1 RCT-49 | 0.9862702 | 1.1859583 | 1.0047666 | 0.8699858 | 1.067766 | 0.8586408 | 1.0070145 |
| Carbonic anhydrase III | 1.2433301 | 1.011188 | 1.8360097 | 1.1873289 | 0.4733803 | 1.3700339 | 1.2391126 |
| Proliferating cell nuclear antigen gene | 1.0001537 | 1.0748879 | 0.9079856 | 0.8603733 | 1.0023931 | 0.8633696 | 0.989206 |
| Integrin beta1 | 1.0537393 | 1.1135844 | 1.1123301 | 1.3409257 | 1.1609395 | 1.1111859 | 1.0403761 |
| Caspase 6 | 0.9479893 | 0.980948 | 1.099585 | 1.0858331 | 1.0522219 | 0.9787135 | 0.9377723 |
| Phase-1 RCT-116 | 1.2129555 | 1.5602555 | 1.1838682 | 1.5093719 | 1.3212972 | 1.0827298 | 1.1557515 |
| Phase-1 RCT-109 | 1.3892442 | 1.4571643 | 1.1545767 | 0.9601951 | 1.1758571 | 1.2127372 | 1.0754565 |
| Phase-1 RCT-71 | 0.978946 | 1.149536 | 1.0938791 | 1.0919402 | 1.4856652 | 1.1384013 | 1.0951779 |
| Elongation factor-1 alpha | 1.2048628 | 1.0291805 | 0.9954457 | 1.3914833 | 1.0896405 | 1.2014773 | 0.9548932 |
| Phase-1 RCT-169 | 0.9256603 | 0.8323792 | 1.0193197 | 1.1016575 | 0.8932812 | 0.9075969 | 1.1343166 |
| Pyruvate kinase, muscle | 0.9381899 | 1.1660395 | 0.9479113 | 1.1126273 | 1.0659025 | 0.9400799 | 0.9464493 |
| Nucleosome assembly protein | 0.9675698 | 1.686649 | 1.3416399 | 0.933803 | 0.7454016 | 1.2980537 | 0.965304 |
| Phase-1 RCT-127 | 1.0005565 | 0.9495652 | 1.0664808 | 0.8375743 | 1.0772692 | 0.8225845 | 1.0849568 |
| Iron-responsive element-binding protein | 1.0185441 | 0.9715442 | 1.0295917 | 1.0631104 | 1.1880623 | 1.0323111 | 1.1156142 |
| Phase-1 RCT-72 | 0.9718688 | 1.0328351 | 1.010514 | 1.0326351 | 0.8817172 | 0.7959827 | 1.0656226 |
| Phase-1 RCT-144 | 1.0570142 | 1.068542 | 0.9395442 | 0.9311223 | 1.1164808 | 0.8473141 | 0.913031 |
| Phase-1 RCT-242 | 0.9874957 | 0.9015144 | 0.9241944 | 0.8375161 | 0.932177 | 0.8002071 | 0.9837781 |
| 17-beta hydroxysteroid dehydrogenase, | 1.0575863 | 2.4837582 | 2.0148299 | 1.0046862 | 0.63258 | 1.5851679 | 1.1880333 |
| type 2 | | | | | | | |
| Phase-1 RCT-70 | 1.1507982 | 1.1338258 | 1.1054069 | 1.0251231 | 1.0979865 | 0.9352164 | 0.9929135 |
| Ribosomal protein L13A | 1.3664813 | 1.4455322 | 1.1761609 | 0.9903457 | 1.0299431 | 1.1326166 | 1.0632643 |
| Cytochrome P450 2E1 | 0.9459211 | 1.1777763 | 1.0346562 | 1.2748483 | 0.3783584 | 1.2014453 | 0.7309387 |
| AT-3 | 0.9239421 | 1.0214568 | 1.0522934 | 0.9826652 | 1.0041664 | 0.9720713 | 0.9276583 |
| Phase-1 RCT-270 | 0.6657512 | 0.47017 | 0.9795018 | 1.4407829 | 1.2062541 | 0.9390898 | 1.1587849 |
| Phase-1 RCT-123 | 0.9675326 | 1.0007063 | 0.9142351 | 0.8138464 | 1.03213 | 0.8974399 | 0.9382651 |
| Alpha-2-macroglobulin, sequence 2 | 0.9797861 | 0.9995096 | 1.0116966 | 1.3936995 | 1.0886897 | 1.0947844 | 1.1405032 |
| Matrix metalloproteinase-1 | 1.0636213 | 0.8628396 | 1.0718575 | 1.1109232 | 0.8888739 | 1.2920443 | 0.8169901 |
| Beta-tubulin, class I | 0.8812037 | 0.8618971 | 0.8316839 | 1.3929935 | 1.3820385 | 1.2515024 | 1.0090874 |
| Phase-1 RCT-161 | 0.9705439 | 1.1194715 | 1.2595773 | 0.7993948 | 0.8937158 | 0.8002781 | 0.7343197 |
| 14-3-3 zeta | 1.0450435 | 1.0489594 | 1.0482322 | 1.0635517 | 1.0038257 | 0.9986565 | 0.9883122 |
| Stem cell factor | 0.8036336 | 0.6085044 | 0.9255235 | 1.378464 | 1.0391836 | 1.0146534 | 1.0026883 |
| Macrophage inflammatory protein-2 alpha | 0.9033192 | 0.7485687 | 0.9462527 | 1.0810547 | 1.1090052 | 1.14337 | 0.8917656 |
| Thymidylate synthase | 1.0589908 | 1.1072227 | 0.945988 | 0.7496661 | 0.8315253 | 0.8798602 | 1.0131876 |
| c-jun | 1.1416692 | 1.0892526 | 1.0810723 | 2.2610528 | 0.9682853 | 0.9591188 | 1.2782836 |
| Superoxide dismutase Mn | 1.2521008 | 1.326852 | 1.0989552 | 1.403422 | 1.2593702 | 1.1555072 | 1.2730436 |
| Phase-1 RCT-73 | 1.1047949 | 0.977787 | 1.0779636 | 1.0577729 | 1.1294429 | 1.0452851 | 1.0321558 |
| Macrophage inflammatory protein-1 alpha | 1.0655606 | 1.0027701 | 1.0328538 | 0.7622603 | 0.7778626 | 0.7866654 | 0.973594 |
| Phase-1 RCT-214 | 0.9631545 | 0.7844238 | 0.8196483 | 0.9291552 | 0.990579 | 0.9888939 | 1.0150075 |
| NADH-cytochrome b5 reductase | 0.8593366 | 0.8004869 | 0.8455348 | 1.1134614 | 1.0447626 | 0.9489074 | 1.1377784 |
| Interleukin-1 beta | 0.9748898 | 0.8820345 | 1.121455 | 1.1273118 | 0.9809169 | 1.1047702 | 0.9470083 |
| NADP-dependent isocitrate | 0.9206625 | 0.6934565 | 0.8368779 | 0.98839 | 1.1040357 | 0.9525166 | 1.1709274 |
| dehydrogenase, cytosolic | | | | | | | |
| Phase-1 RCT-289 | 1.0622177 | 0.9919961 | 1.2031344 | 0.9698447 | 0.9422024 | 1 | 0.7444939 |
| Glutamine synthetase | 0.8225883 | 1.0531967 | 0.8729375 | 1.0046906 | 0.7456022 | 0.9876058 | 1.1271669 |
| Phase-1 RCT-182 | 0.8362826 | 0.8982307 | 1.0926597 | 0.9732112 | 0.8329441 | 1.1651516 | 0.8792288 |
| Ubiquitin conjugating enzyme (RAD 6 | 1.1046615 | 0.9266716 | 1.0462561 | 0.9350017 | 0.8941758 | 1.2123113 | 1.1891202 |
| homologue) | | | | | | | |
| Extracellular-signal-regulated kinase 1 | 0.900061 | 1.0584913 | 1.2510608 | 1.1057161 | 0.6285249 | 1.2222856 | 0.9560394 |
| Emerin | 1.114966 | 1.3014317 | 1.444523 | 0.9345439 | 1.0051866 | 0.9867944 | 1.0956088 |
| Phase-1 RCT-78 | 0.8533472 | 0.9828223 | 1.0001426 | 1.0772234 | 1.127215 | 1.0338076 | 0.9598559 |
| Phase-1 RCT-212 | 0.9593411 | 0.8509183 | 0.9671716 | 0.9144535 | 0.8559242 | 0.9199735 | 1.1306477 |
| Phase-1 RCT-168 | 1.104003 | 1.0789691 | 1.1120517 | 1.0144646 | 0.9892755 | 1.0412582 | 1.0742358 |
| Lysyl hydroxylase | 0.9895941 | 1.0065663 | 1.0063249 | 0.9174858 | 0.6834018 | 1.0229279 | 0.8805752 |
| Phase-1 RCT-55 | 0.9402393 | 1.3462903 | 0.8970724 | 0.9672162 | 0.7208364 | 0.7303916 | 1.0411817 |
| MHC class I antigen RT1.A1(f) alpha- | 1.0314152 | 1.1438619 | 0.9624206 | 1.1996666 | 1.4604423 | 1.0184325 | 0.9979649 |
| chain | | | | | | | |
| Phase-1 RCT-40 | 0.6989399 | 0.6283605 | 0.8560695 | 1.572512 | 1.6342572 | 0.9743063 | 0.8613499 |
| Cyclin G | 0.9674813 | 1.4618573 | 1.0295708 | 1.0565988 | 0.8900976 | 1.071694 | 1.0041132 |
| Peroxisome proliferator activated receptor | 0.9929379 | 1.3650321 | 0.8849561 | 0.8480831 | 0.6568345 | 0.9971213 | 1.016789 |
| gamma | | | | | | | |
| Phase-1 RCT-280 | 1.0198756 | 0.8127384 | 0.8144099 | 0.9713171 | 0.8575961 | 1.0165477 | 0.9381239 |
| D-dopachrome tautomerase | 0.8693069 | 0.8125631 | 0.9842208 | 1.1838523 | 1 | 1.2375549 | 1.232398 |
| Phase-1 RCT-287 | 1.2267436 | 1.0080564 | 1.1650683 | 1.1046329 | 1.171368 | 0.9995269 | 0.9099843 |
| Phase-1 RCT-119 | 1.0445286 | 0.9946325 | 1.0852857 | 1.1677629 | 0.9518777 | 1.0818635 | 0.8382615 |
| Thioredoxin-1 (Trx1) | 1.1610906 | 1.0985358 | 1.0781599 | 1.4042255 | 1.3907342 | 1.0980104 | 1.2127857 |
| Phase-1 RCT-225 | 0.9116357 | 1.4018592 | 0.6712688 | 0.2718085 | 0.579256 | 1.1403403 | 0.6495857 |
| Protein kinase C alpha | 0.9576035 | 0.988513 | 0.969444 | 1.0275874 | 0.9313216 | 1.00167 | 0.9176223 |
| Phase-1 RCT-87 | 1.0210327 | 0.86518 | 1.0520636 | 0.9509893 | 1.0212699 | 0.8242882 | 0.8024983 |
| Phase-1 RCT-59 | 1.077258 | 1.0592402 | 1.235905 | 0.690686 | 0.9899694 | 0.8262069 | 0.9104352 |
| C4b-binding protein | 0.7414722 | 0.523028 | 0.775379 | 1.0130826 | 1.3614047 | 1.1229963 | 0.831654 |
| Phase-1 RCT-204 | 0.9753441 | 1.0067638 | 0.9410997 | 0.9762682 | 1.0234381 | 1.0556935 | 1.2071922 |
| Carnitine palmitoyl-CoA transferase | 1.0340303 | 1.7449958 | 1.3448441 | 0.9355328 | 1.051204 | 1.1397537 | 0.8802019 |
| 8-oxoguanine DNA glycosylase | 0.8450393 | 1.0108324 | 0.863728 | 0.9439642 | 0.901842 | 0.8836787 | 1.0015395 |
| H-rev107 | 1.0062102 | 0.9867915 | 0.8229442 | 0.8214302 | 0.8954701 | 0.8498508 | 0.9250225 |
| Preproalbumin | 1.0630776 | 0.8958946 | 0.9429222 | 1.3287193 | 1.5401993 | 1.3660408 | 1.2189285 |
| Phase-1 RCT-177 | 1.3284144 | 0.8756865 | 1.0373514 | 1.2515868 | 1.4211255 | 1.1879334 | 1.1836313 |
| Phase-1 RCT-110 | 0.9262117 | 1.1018304 | 0.9765634 | 0.735895 | 0.8863279 | 0.7774005 | 0.8361396 |
| Phase-1 RCT-227 | 0.9089461 | 1.1239718 | 1.0288868 | 1.2899691 | 1.0213081 | 1.311045 | 1.3450123 |
| Neuropeptide Y | 0.9438619 | 0.9527084 | 0.8020995 | 0.8628566 | 0.9022168 | 0.8442031 | 0.8854681 |
| Phase-1 RCT-277 | 0.7125464 | 0.5758576 | 0.6648771 | 1.0231367 | 1.1316079 | 1.0646178 | 1.1152446 |
| Phase-1 RCT-229 | 0.8970875 | 1.0555822 | 1.0472574 | 0.8644863 | 0.867917 | 0.8393984 | 0.9327256 |
| Phase-1 RCT-34 | 0.9842579 | 1.2005953 | 1.1281708 | 2.0779154 | 1.1169853 | 1.3680507 | 1.2224481 |
| Choline kinase | 0.904444 | 0.9331387 | 1.0942782 | 1.4028679 | 1.3247349 | 0.6788527 | 0.9972829 |
| Phase-1 RCT-20 | 1.0025303 | 1.1695054 | 0.9887838 | 1.0598549 | 1.0337205 | 0.9270567 | 1.040153 |
| Phase-1 RCT-248 | 1.0539098 | 1.2938945 | 1.1279898 | 0.7711037 | 0.889462 | 0.5159522 | 0.9320796 |
| | CLOZ | CLOZ | CMC | CMC | CMC | CHEX | CHEX |
| Compound-Dose (2) | 180 | 180 | 30 | 30 | 30 | 0.5 | 0.5 |
| Animal Number (3) | 2432 | 2433 | 151 | 152 | 153 | 2241 | 2242 |
| Liver Toxicity Necrosis Classification (4) | no | no | no | no | no | no | no |
| Gene Name (5) | | | | | | | |
| Gadd153 | 1.1765646 | 0.9342311 | 0.7606967 | 0.8100289 | 1.0811524 | 1.9686092 | 1.385269 |
| Interferon related developmental regulator | 1.0345932 | 1.0207139 | 0.7855883 | 0.8484688 | 0.773653 | 1.5959315 | 1.3303684 |
| IFRD1 (PC4) | | | | | | | |
| ID-1 | 1.039665 | 1.1181648 | 0.8002384 | 0.9862174 | 0.9970438 | 1.3464538 | 1.5311825 |
| Insulin-like growth factor binding protein 1 | 1.122365 | 0.8346951 | 1.0216945 | 0.9608531 | 0.985763 | 0.8641444 | 1.4837782 |
| NIPK | 1.0983853 | 1.0368168 | 1.0869685 | 1.0931784 | 1.0450101 | 1.1963465 | 1.1277021 |
| Phase-1 RCT-50 | 1.0112197 | 0.9465407 | 1.0047923 | 1.1946847 | 1.3816605 | 4.033162 | 5.585607 |
| Sarcoplasmic reticulum calcium ATPase | 1.0298532 | 1.0411139 | 0.5761599 | 0.6227019 | 0.9184884 | 1.2259504 | 1.1588426 |
| Phase-1 RCT-207 | 1.0310225 | 0.9897835 | 1.160821 | 1.1341091 | 0.9207895 | 2.7614477 | 2.5460906 |
| Acetyl-CoA carboxylase | 0.9105415 | 1.0918167 | 0.9740571 | 0.9380899 | 0.9710398 | 0.9110474 | 0.8647038 |
| Cathepsin L, sequence 2 | 1.1793472 | 1.5047537 | 0.7683697 | 0.9822233 | 0.6843788 | 2.7601736 | 3.3044343 |
| Glucokinase | 0.6881666 | 0.5475149 | 0.3774918 | 0.4271972 | 0.4861491 | 0.6511732 | 0.5061088 |
| Gadd45 | 0.9435921 | 1.5789286 | 1.2575942 | 1.8472987 | 1.2860641 | 1.214662 | 1 |
| Phase-1 RCT-18 | 0.8677251 | 0.8760584 | 1.0255771 | 1.1607585 | 1.4343021 | 1.034599 | 1.0540371 |
| 3-beta-hydroxysteroid dehydrogenase | 1.0123714 | 1.0091615 | 0.8306232 | 1.0016026 | 0.9553083 | 0.7582176 | 0.8239129 |
| (HSD3B1) | | | | | | | |
| Phase-1 RCT-221 | 1.0090134 | 1.1305184 | 0.8779668 | 0.9119122 | 0.7612242 | 0.8988828 | 1.1127518 |
| Heme oxygenase | 1.1148443 | 1.3064055 | 1.4164264 | 1.2920971 | 0.9538655 | 0.7792441 | 0.8337236 |
| Cyclin dependent kinase 4 | 1.0413052 | 0.9474883 | 0.7992693 | 0.7279431 | 0.871404 | 0.9606662 | 0.7006463 |
| Phase-1 RCT-75 | 0.9723821 | 1.0765303 | 1.1710882 | 1.1886568 | 1.0708501 | 1.8986644 | 1.8782042 |
| Phase-1 RCT-65 | 1.0902854 | 1.1426122 | 1.181896 | 1.3632299 | 1.2637658 | 1.084024 | 0.9382915 |
| Cytochrome P450 1A1 | 0.9485354 | 0.7873413 | 0.8936267 | 1.1261358 | 1.0374235 | 1.2725967 | 1.2586222 |
| Peroxisomal multifunctional enzyme type II | 1.0240006 | 1.4589493 | 1.1908965 | 1.155684 | 1.0523934 | 0.7897263 | 0.791615 |
| DNA topoisomerase I | 1.2072941 | 1.3180852 | 0.8825739 | 0.821817 | 0.6059353 | 0.8200669 | 0.7568441 |
| Focal adhesion kinase (pp125FAK) | 0.9397891 | 0.8956956 | 0.9151267 | 1.0396539 | 1.0989453 | 1.249245 | 1.3170962 |
| NGF-inducible anti-proliferative putative | 0.9143484 | 0.9376797 | 1.2660489 | 1.1596255 | 1.4273437 | 1.8212605 | 1.5847789 |
| secreted protein (PC3) | | | | | | | |
| Phase-1 RCT-82 | 0.970567 | 0.8449153 | 0.9342508 | 0.9788081 | 1.177113 | 0.9320096 | 0.8798012 |
| Senescence marker protein-30 | 1.0866205 | 0.7431943 | 0.6006578 | 0.529814 | 0.6655551 | 0.3749239 | 0.1939765 |
| Melanoma-associated antigen ME491 | 0.9170407 | 0.9549704 | 0.7661839 | 0.7212641 | 0.9350305 | 1.1566821 | 1.3748273 |
| Activating transcription factor 3 | 0.9989253 | 0.9460117 | 1.2127811 | 1.1242064 | 1.2143363 | 0.8549494 | 0.8087356 |
| Tryptophan hydroxylase | 0.9798663 | 1.2962693 | 1.119373 | 0.9010423 | 0.8489122 | 0.6452963 | 0.5691863 |
| Zinc finger protein | 1.1548481 | 0.8096424 | 1.1444403 | 1.0999786 | 1.0347527 | 3.484682 | 4.6837034 |
| c-myc | 3.2173533 | 0.8386362 | 1.2135689 | 1.3119427 | 1.4491754 | 1.5442644 | 2.188976 |
| Cholesterol 7-alpha-hydroxylase (P450 | 1.1206236 | 0.7854834 | 0.746895 | 0.6386592 | 0.7229146 | 0.9487857 | 0.8408052 |
| VII) | | | | | | | |
| Ferritin H-chain | 1.024457 | 1.1885884 | 0.900636 | 0.8596141 | 0.8203639 | 0.6596249 | 0.6648522 |
| Phase-1 RCT-197 | 0.9510899 | 1.0383345 | 0.8136439 | 1.0090666 | 0.9463343 | 1.422632 | 1.3185365 |
| Phase-1 RCT-179 | 1.0944417 | 1.3954943 | 0.8824895 | 1.0650761 | 0.6717649 | 2.000525 | 2.1926706 |
| Phase-1 RCT-15 | 1.3845695 | 1.6682378 | 1.0426614 | 1.1671517 | 1.2003523 | 2.660902 | 2.6730523 |
| Argininosuccinate lyase | 1.3072342 | 1.5775527 | 1.0951878 | 1.0512652 | 0.9066866 | 1.7755879 | 2.3974206 |
| Phase-1 RCT-49 | 1.0144081 | 1.0287235 | 0.9820635 | 1.1869719 | 0.9786616 | 3.088243 | 4.431734 |
| Carbonic anhydrase III | 0.7080345 | 0.4872412 | 0.6890884 | 0.6857857 | 1.1619563 | 0.2251441 | 0.0473048 |
| Proliferating cell nuclear antigen gene | 1.0091459 | 0.9085991 | 1.3036764 | 1.154817 | 1.3424358 | 1.218333 | 1.1726964 |
| Integrin beta1 | 1.0186528 | 1.0347819 | 0.9667066 | 1.0861601 | 1.4365923 | 1.6006643 | 1.7452571 |
| Caspase 6 | 0.9596847 | 0.9224901 | 0.8707469 | 0.8704625 | 1.3079352 | 1.22215 | 1.0536768 |
| Phase-1 RCT-116 | 1.4288309 | 1.2664233 | 1.013206 | 1.2134981 | 0.9774803 | 1.112274 | 1.0605539 |
| Phase-1 RCT-109 | 1.0705416 | 1.0619936 | 0.781185 | 0.892063 | 0.6276345 | 1.2626413 | 1.2305433 |
| Phase-1 RCT-71 | 1.0750757 | 1.1083864 | 1.0887866 | 1.2617254 | 1.2610478 | 2.0775554 | 2.009428 |
| Elongation factor-1 alpha | 1.0186756 | 1.1598856 | 0.6795511 | 0.7591885 | 0.5408555 | 0.9531817 | 0.9033678 |
| Phase-1 RCT-169 | 0.9489923 | 0.8144562 | 1.324167 | 1 | 1.2558181 | 1.2609965 | 1.1902367 |
| Pyruvate kinase, muscle | 0.9847708 | 1.2753224 | 0.8729103 | 1.069449 | 0.987743 | 2.0013642 | 2.4644535 |
| Nucleosome assembly protein | 0.9594558 | 0.6831263 | 1.2755677 | 1.4252563 | 1.5193342 | 0.6012471 | 0.6260324 |
| Phase-1 RCT-127 | 1.0100638 | 1.1774249 | 1.1844738 | 1.1618335 | 0.9404816 | 2.8177075 | 3.3913255 |
| Iron-responsive element-binding protein | 1.0244161 | 1.0808064 | 0.9994935 | 0.8113191 | 0.9684551 | 0.6414047 | 0.5316567 |
| Phase-1 RCT-72 | 0.9228984 | 0.8384938 | 0.9370267 | 1.0207202 | 1.0474843 | 1.5537046 | 1.2441758 |
| Phase-1 RCT-144 | 1.0675185 | 1.2196552 | 0.7341973 | 0.8196614 | 0.6286161 | 2.5857449 | 3.6033442 |
| Phase-1 RCT-242 | 1.063435 | 0.8936893 | 1.1210269 | 1.3456041 | 1.4646462 | 3.2036362 | 3.7286282 |
| 17-beta hydroxysteroid dehydrogenase, | 1.1439115 | 0.5170498 | 2.3895397 | 2.3121948 | 2.2213478 | 0.3941614 | 0.4124797 |
| type 2 | | | | | | | |
| Phase-1 RCT-70 | 0.9604012 | 0.9166661 | 1.0521004 | 1.0162028 | 1.0391489 | 1.3115969 | 1.3713875 |
| Ribosomal protein L13A | 1.1162372 | 1.1584325 | 0.8795698 | 0.9925988 | 0.5878612 | 1.2140301 | 1.2934426 |
| Cytochrome P450 2E1 | 0.9701947 | 0.9544632 | 1.1509818 | 1.1771178 | 1.0047926 | 1.119516 | 1.6182413 |
| AT-3 | 0.8523181 | 0.9310506 | 0.8327506 | 0.9218012 | 1.0671057 | 1.022456 | 1.090334 |
| Phase-1 RCT-270 | 0.8684931 | 0.8844253 | 0.9597781 | 0.7339256 | 0.9525176 | 0.3805625 | 0.3087489 |
| Phase-1 RCT-123 | 0.8708859 | 0.8487685 | 1.1584042 | 1.1991154 | 1.1339612 | 0.8036441 | 0.8162525 |
| Alpha-2-macroglobulin, sequence 2 | 1.1571331 | 1.2587081 | 1.0136857 | 0.9132677 | 1.055114 | 1.0750335 | 1.1854357 |
| Matrix metalloproteinase-1 | 0.9097909 | 1.1867009 | 1.0106953 | 0.9432278 | 0.9539881 | 0.8893518 | 1.0111585 |
| Beta-tubulin, class I | 1.2194175 | 1.0281503 | 0.6825807 | 0.8112711 | 0.5381852 | 0.9494259 | 0.9725797 |
| Phase-1 RCT-161 | 0.9299735 | 0.7980477 | 0.8477458 | 1.0113993 | 1.4442161 | 1.0018919 | 0.9606074 |
| 14-3-3 zeta | 0.9489332 | 0.9815716 | 1.0571456 | 1.0229127 | 0.9634926 | 1.5139843 | 1.4217017 |
| Stem cell factor | 0.9766085 | 0.796292 | 0.6532143 | 0.5942181 | 0.8936085 | 0.9135138 | 0.7087905 |
| Macrophage inflammatory protein-2 alpha | 0.9840262 | 0.9278642 | 0.961556 | 1.5673363 | 1.6591642 | 1.6509347 | 1.8104758 |
| Thymidylate synthase | 0.891104 | 0.7403107 | 1.1896576 | 1.3338974 | 1.6733768 | 1.145832 | 1.0966454 |
| c-jun | 0.9436312 | 0.9767608 | 1.1291357 | 1.4380051 | 1.3538828 | 2.3322034 | 1.587437 |
| Superoxide dismutase Mn | 1.1855384 | 1.1897198 | 1.1143311 | 0.9802153 | 1.0487516 | 2.2193022 | 1.4983441 |
| Phase-1 RCT-73 | 0.9962387 | 1.1204464 | 1.03073 | 1.0239667 | 1.1554004 | 0.7492527 | 0.8100732 |
| Macrophage inflammatory protein-1 alpha | 1.2308886 | 0.7427456 | 1.2069107 | 1.1516151 | 1.3781619 | 1.254737 | 1.4303427 |
| Phase-1 RCT-214 | 1.1070365 | 0.9745829 | 1.1208043 | 0.8715661 | 1.0046376 | 0.6881101 | 0.5054577 |
| NADH-cytochrome b5 reductase | 0.9820256 | 0.9807084 | 1.1736493 | 0.7969866 | 1.1578748 | 0.3745182 | 0.3558243 |
| Interleukin-1 beta | 0.9571706 | 1.1233792 | 0.9544559 | 0.9596923 | 1.0394517 | 0.895968 | 0.9455692 |
| NADP-dependent isocitrate | 0.9243189 | 0.8436446 | 1.0873817 | 0.7924356 | 0.8468297 | 0.2760605 | 0.2540225 |
| dehydrogenase, cytosolic | | | | | | | |
| Phase-1 RCT-289 | 0.8873094 | 0.84607 | 1.0276419 | 0.9656249 | 0.9856671 | 0.560843 | 0.5658117 |
| Glutamine synthetase | 0.9864295 | 0.8659521 | 0.957763 | 0.7906025 | 0.8461093 | 1.2062832 | 1.2588074 |
| Phase-1 RCT-182 | 0.8127571 | 1.0544989 | 1.0046797 | 0.8246641 | 0.8762103 | 0.3865644 | 0.4196328 |
| Ubiquitin conjugating enzyme (RAD 6 | 1.1756055 | 1.4245914 | 0.8570273 | 0.8660294 | 0.6684883 | 0.9942962 | 1.247427 |
| homologue) | | | | | | | |
| Extracellular-signal-regulated kinase 1 | 0.6398795 | 0.5311057 | 0.6864108 | 0.739759 | 1.1879661 | 0.3524557 | 0.2236294 |
| Emerin | 1.0733517 | 0.9704013 | 1.185682 | 1.1641341 | 1.1485847 | 0.8379549 | 0.7791347 |
| Phase-1 RCT-78 | 0.8696312 | 0.8977453 | 1.1021167 | 0.9108863 | 1.0313257 | 0.8074775 | 0.7246457 |
| Phase-1 RCT-212 | 0.9945802 | 1.0897845 | 1.0095264 | 1.0338799 | 0.9202671 | 0.9132324 | 0.890877 |
| Phase-1 RCT-168 | 0.8264778 | 0.9121315 | 1.0077223 | 0.9935367 | 0.8917636 | 0.517829 | 0.5605307 |
| Lysyl hydroxylase | 0.94825 | 0.9931779 | 0.8755615 | 0.7603325 | 0.8423739 | 1.0575664 | 1.0978385 |
| Phase-1 RCT-55 | 0.9595241 | 0.9942521 | 0.9220764 | 0.8947722 | 0.9568933 | 1.121799 | 1.4907624 |
| MHC class I antigen RT1.A1(f) alpha- | 1.1783234 | 1.1231898 | 1.1453495 | 1.1601518 | 1.6179262 | 1.452018 | 1.2771279 |
| chain | | | | | | | |
| Phase-1 RCT-40 | 0.896767 | 0.9688458 | 1.0283419 | 1.1065841 | 0.9202671 | 0.5696715 | 0.4838135 |
| Cyclin G | 1.0827142 | 1.1595474 | 1.034909 | 1.2865733 | 1.2463012 | 3.7452185 | 4.571906 |
| Peroxisome proliferator activated receptor | 0.8723482 | 1.345292 | 1.4028924 | 1.2013322 | 1.5603776 | 1.1553857 | 0.970739 |
| gamma | | | | | | | |
| Phase-1 RCT-280 | 1.4093803 | 0.8843707 | 1.1513247 | 1.0119642 | 1.0737574 | 0.7020422 | 0.4806872 |
| D-dopachrome tautomerase | 0.9785563 | 1.1540986 | 0.9527921 | 0.9071145 | 0.6776088 | 0.3796619 | 0.3768816 |
| Phase-1 RCT-287 | 0.9658876 | 1.0431648 | 0.7402771 | 0.7400485 | 0.9174875 | 0.7734382 | 0.7382091 |
| Phase-1 RCT-119 | 0.9308044 | 1.1316528 | 1.0356885 | 0.9959465 | 1.3149339 | 0.9015281 | 1.2205942 |
| Thioredoxin-1 (Trx1) | 1.0960408 | 1.0716466 | 1.2250621 | 1.1472393 | 1.0255165 | 0.6609843 | 0.637054 |
| Phase-1 RCT-225 | 0.8276014 | 0.9337168 | 0.7480868 | 0.8256199 | 0.2722594 | 1.4610342 | 1.2179135 |
| Protein kinase C alpha | 0.8718494 | 0.8583676 | 0.7528158 | 0.6720756 | 0.7777195 | 1.125374 | 1.1235071 |
| Phase-1 RCT-87 | 0.8772966 | 0.9689744 | 1.1319923 | 1.0914907 | 1.0641888 | 0.7451364 | 0.734158 |
| Phase-1 RCT-59 | 1.108546 | 0.8209917 | 0.9071966 | 1.0232013 | 0.905435 | 2.5667937 | 2.6899319 |
| C4b-binding protein | 0.9751314 | 1.134305 | 0.9225394 | 1.0717598 | 0.8385773 | 0.7869753 | 0.6623135 |
| Phase-1 RCT-204 | 1.1083045 | 1.3348511 | 0.9210352 | 0.9427806 | 0.9680378 | 1.6626132 | 2.26592 |
| Carnitine palmitoyl-CoA transferase | 1.1462771 | 1.3422669 | 1.3206326 | 1.3679037 | 1.5878874 | 1.3934232 | 1.3271409 |
| 8-oxoguanine DNA glycosylase | 1.0086834 | 0.9101519 | 1.153777 | 1.0768057 | 1.2754111 | 1.3796998 | 1.479251 |
| H-rev107 | 0.9197654 | 0.3972068 | 1.1598829 | 1.1856582 | 1.0389929 | 1.1313727 | 1.2012417 |
| Preproalbumin | 1.0872719 | 1.2314241 | 1.1835746 | 1.113928 | 1.4968722 | 0.4791996 | 0.31543 |
| Phase-1 RCT-177 | 1.2105507 | 1.3585994 | 1.061051 | 0.9954885 | 0.9349856 | 0.6427981 | 0.6380794 |
| Phase-1 RCT-110 | 0.8840316 | 0.8710881 | 1.1295111 | 1.3195572 | 1.6803578 | 1.3594975 | 1.0384028 |
| Phase-1 RCT-227 | 0.8812623 | 1.2222947 | 1.2152252 | 0.9932076 | 1.1232198 | 0.5600708 | 0.4153755 |
| Neuropeptide Y | 1.4324944 | 0.804572 | 0.9906946 | 1.1596292 | 1.3258679 | 0.9655659 | 0.8575395 |
| Phase-1 RCT-277 | 1.0031583 | 0.9726225 | 1.2481302 | 1.2206075 | 1.2734294 | 0.5275346 | 0.5100324 |
| Phase-1 RCT-229 | 0.8779392 | 0.848289 | 1.0534588 | 1.0704379 | 1.3080869 | 1.0797726 | 0.9444139 |
| Phase-1 RCT-34 | 1.092193 | 1.1345469 | 1.2249236 | 0.9222522 | 0.9998194 | 0.5974576 | 0.5671926 |
| Choline kinase | 1.0185006 | 0.848261 | 0.8494175 | 0.7929388 | 1.2738152 | 1.3471842 | 1.3503621 |
| Phase-1 RCT-20 | 0.983609 | 0.9849164 | 1.0130256 | 1.0932242 | 1.2552941 | 1.1336882 | 1.0829953 |
| Phase-1 RCT-248 | 0.9497179 | 0.7653366 | 0.9090595 | 1.0039512 | 0.7945378 | 1.0558136 | 1.1855242 |
| | CHEX | CHEX | CHEX | CHEX | CPHOS | CPHOS | CPHOS |
| Compound-Dose (2) | 0.5 | 2 | 2 | 2 | 25 | 25 | 25 |
| Animal Number (3) | 2243 | 2251 | 2252 | 2253 | 2141 | 2142 | 2143 |
| Liver Toxicity Necrosis Classification (4) | no | no | no | no | no | no | no |
| Gene Name (5) | | | | | | | |
| Gadd153 | 1.7844477 | 1.323875 | 1.5871651 | 1.8510119 | 1.0268688 | 1.1817113 | 1.3905258 |
| Interferon related developmental regulator | 1.3230062 | 1.1131834 | 1.2424812 | 1.5277249 | 1.1144816 | 1.2480401 | 0.791119 |
| IFRD1 (PC4) | | | | | | | |
| ID-1 | 1.5403085 | 0.9215258 | 1.0406017 | 0.9977781 | 1.0737023 | 1.218934 | 1.656492 |
| Insulin-like growth factor binding protein 1 | 0.9697186 | 8.189722 | 36.193382 | 18.845623 | 1.2939781 | 1.5581142 | 1.086005 |
| NIPK | 1.0629793 | 1.2139448 | 1.4987214 | 1.2468987 | 1.1163266 | 1.1450868 | 1.1998578 |
| Phase-1 RCT-50 | 3.449078 | 4.107797 | 7.0997186 | 8.844672 | 0.8369129 | 0.935373 | 0.8181895 |
| Sarcoplasmic reticulum calcium ATPase | 1.2380323 | 0.9488905 | 0.8117185 | 0.8171244 | 0.8859316 | 0.8926636 | 1.0207125 |
| Phase-1 RCT-207 | 2.2760494 | 3.0277555 | 4.7050414 | 4.5417094 | 1.0999082 | 1.2015456 | 1.3157319 |
| Acetyl-CoA carboxylase | 0.8709167 | 0.9954698 | 0.7451589 | 0.7324497 | 1.0301207 | 0.8291789 | 1.0908287 |
| Cathepsin L, sequence 2 | 3.1731493 | 2.968516 | 5.7428937 | 4.2624383 | 1.2560953 | 1.387408 | 0.7994248 |
| Glucokinase | 0.8462257 | 0.5817117 | 0.4505983 | 0.3842188 | 0.9972352 | 0.6873238 | 2.2650378 |
| Gadd45 | 1.0798941 | 8.564091 | 9.897914 | 13.841803 | 1.4409771 | 1.6393377 | 1.5678291 |
| Phase-1 RCT-18 | 1.0813061 | 0.9127868 | 0.8315097 | 0.8734788 | 0.9405059 | 0.9961301 | 1.0864013 |
| 3-beta-hydroxysteroid dehydrogenase | 0.6911956 | 0.956894 | 0.9700759 | 1.0063759 | 0.8148979 | 0.7476584 | 0.7089165 |
| (HSD3B1) | | | | | | | |
| Phase-1 RCT-221 | 0.9522474 | 1.1702023 | 1.0630686 | 1.0842638 | 1.0972105 | 1.2254709 | 1.0788829 |
| Heme oxygenase | 0.9211622 | 1.321163 | 0.9363931 | 1.0621456 | 1.0715398 | 2.067676 | 0.952763 |
| Cyclin dependent kinase 4 | 1.1203977 | 0.9351213 | 0.7578149 | 0.8261299 | 1.0173368 | 0.865562 | 1.0408356 |
| Phase-1 RCT-75 | 2.068471 | 1.517875 | 3.072005 | 2.5647874 | 0.9999719 | 0.9432594 | 1.2245024 |
| Phase-1 RCT-65 | 1.0132 | 1.570903 | 1.2304034 | 2.1907747 | 1.3841032 | 1.4658718 | 1.7493815 |
| Cytochrome P450 1A1 | 1.1925737 | 0.875699 | 0.804912 | 0.9034701 | 0.9763113 | 0.9893409 | 1.6522053 |
| Peroxisomal multifunctional enzyme type II | 0.8538019 | 1.0089917 | 1.2196586 | 1.1796415 | 0.7978566 | 0.7572708 | 0.636024 |
| DNA topoisomerase I | 0.7561094 | 1.3112004 | 1.7509251 | 1.6573284 | 1.0103524 | 0.95232 | 0.7057418 |
| Focal adhesion kinase (pp125FAK) | 1.173637 | 1.1720437 | 1.242579 | 1.240816 | 0.9108803 | 0.9569864 | 0.9533665 |
| NGF-inducible anti-proliferative putative | 1.3271519 | 5.7076497 | 5.450073 | 7.0906177 | 0.8127426 | 0.8492096 | 0.7577347 |
| secreted protein (PC3) | | | | | | | |
| Phase-1 RCT-82 | 0.9313718 | 0.91627 | 0.8603264 | 0.8793117 | 0.8505064 | 0.8814123 | 1.1427218 |
| Senescence marker protein-30 | 0.5128356 | 0.7268904 | 0.2625777 | 0.3415793 | 0.7898583 | 0.6336256 | 0.5463171 |
| Melanoma-associated antigen ME491 | 1.458863 | 1.2571139 | 1.358107 | 1.8041399 | 0.8524907 | 0.8168763 | 0.9693549 |
| Activating transcription factor 3 | 0.6788724 | 2.1682773 | 2.4969354 | 1.606443 | 1.1929091 | 1.4835409 | 1.4524212 |
| Tryptophan hydroxylase | 0.7219233 | 0.894258 | 0.8631896 | 0.8110071 | 0.9884976 | 0.8817688 | 0.9493015 |
| Zinc finger protein | 3.4983404 | 5.8323665 | 20.65312 | 16.238869 | 0.9671843 | 0.8700054 | 0.8209398 |
| c-myc | 1.3780406 | 2.6990962 | 4.500065 | 5.7699766 | 1.1795896 | 1.1368349 | 1.5797365 |
| Cholesterol 7-alpha-hydroxytase (P450 | 1.0106151 | 0.9307327 | 0.5551805 | 0.7264097 | 0.7046924 | 0.8406197 | 0.7502559 |
| VII) | | | | | | | |
| Ferritin H-chain | 1.120655 | 1.0574666 | 1.3032757 | 1.1911676 | 1.7259506 | 2.124993 | 1.3000276 |
| Phase-1 RCT-197 | 1.1259809 | 1.1483266 | 1.0638589 | 1.099802 | 0.9297582 | 1.0033861 | 0.9800899 |
| Phase-1 RCT-179 | 2.3926995 | 1.9514495 | 3.7769787 | 3.514739 | 0.8294568 | 0.8853722 | 0.600083 |
| Phase-1 RCT-15 | 2.389244 | 2.4877126 | 3.2787433 | 4.6626782 | 1.0189652 | 1.2284777 | 1.3648192 |
| Argininosuccinate lyase | 2.6214097 | 1.5724729 | 3.2236745 | 2.7559874 | 1.36262 | 1.4035317 | 1.5341467 |
| Phase-1 RCT-49 | 3.648836 | 3.0263925 | 3.6449368 | 5.7354307 | 1.0370276 | 1.096836 | 1.1717484 |
| Carbonic anhydrase III | 0.2040876 | 1.052352 | 0.1343771 | 0.1308409 | 0.6068291 | 0.3565591 | 0.9198937 |
| Proliferating cell nuclear antigen gene | 1.1873177 | 1.0491829 | 1.1203165 | 1.1143941 | 0.9657635 | 1.0552827 | 1.0787797 |
| Integrin beta1 | 1.5779473 | 2.133643 | 3.3411343 | 4.0190945 | 1.2312363 | 1.3565775 | 1.1215075 |
| Caspase 6 | 1.2755684 | 1.0899495 | 1.256474 | 1.3291011 | 1.0951895 | 1.1519257 | 1.0309988 |
| Phase-1 RCT-116 | 0.9908677 | 1.1092023 | 1.309892 | 1.1826066 | 1.093061 | 1.5732199 | 1.3841608 |
| Phase-1 RCT-109 | 1.0133312 | 0.7945779 | 0.6877067 | 0.816251 | 1.4624946 | 1.7340869 | 1.232109 |
| Phase-1 RCT-71 | 1.5412561 | 1.4400856 | 2.304412 | 2.602543 | 0.9690542 | 1.0674455 | 0.984124 |
| Elongation factor-1 alpha | 1.2136638 | 1.1315401 | 1.4706717 | 1.2901378 | 1.1241682 | 1.014994 | 0.7034068 |
| Phase-1 RCT-169 | 1.208126 | 3.927512 | 2.0512316 | 5.9334583 | 0.863557 | 1.0413496 | 1.3507943 |
| Pyruvate kinase, muscle | 2.474455 | 2.0192885 | 3.5376008 | 2.9893672 | 1.2199836 | 1.2529191 | 1.0947062 |
| Nucleosome assembly protein | 0.8932825 | 0.8199922 | 0.9084436 | 0.8458162 | 1.1175299 | 0.9906919 | 0.5912249 |
| Phase-1 RCT-127 | 3.8954964 | 1.4004384 | 2.4070141 | 2.910667 | 1.00687 | 1.0534936 | 1.1599785 |
| Iron-responsive element-binding protein | 0.6725869 | 0.7815192 | 0.4980847 | 0.4682379 | 0.7967762 | 0.6607793 | 0.6482077 |
| Phase-1 RCT-72 | 1.0877193 | 0.9645977 | 0.9928149 | 0.9518874 | 0.8207147 | 0.8663458 | 1.1775148 |
| Phase-1 RCT-144 | 3.1176043 | 2.4285176 | 3.5942986 | 4.4746027 | 0.8841828 | 1.0156157 | 1.0168767 |
| Phase-1 RCT-242 | 2.3161387 | 6.8508797 | 4.933473 | 4.459887 | 0.9354186 | 1.0391505 | 1.4243038 |
| 17-beta hydroxysteroid dehydrogenase, | 0.8965371 | 0.3727773 | 0.5286655 | 0.518855 | 1.0351092 | 0.9963641 | 0.4123494 |
| type 2 | | | | | | | |
| Phase-1 RCT-70 | 1.1466987 | 1.172485 | 1.1482631 | 1.1419212 | 0.9596515 | 1.1593261 | 1.9536109 |
| Ribosomal protein L13A | 1.3171009 | 1.1635101 | 1.8168858 | 1.5273411 | 1.7436887 | 2.1965175 | 1.2156801 |
| Cytochrome P450 2E1 | 1.3057531 | 0.8478242 | 0.5055624 | 0.8444709 | 0.7969885 | 0.6166129 | 0.5902626 |
| AT-3 | 1.1938004 | 1.0578861 | 1.2179605 | 1.1744331 | 0.8774751 | 0.9185972 | 1.039477 |
| Phase-1 RCT-270 | 0.4908581 | 0.9087259 | 0.3759474 | 0.2921523 | 0.8298319 | 0.5071461 | 0.8152669 |
| Phase-1 RCT-123 | 1.0860355 | 0.9285832 | 1.1748255 | 1.0117396 | 0.9511458 | 0.9128588 | 1.1499667 |
| Alpha-2-macroglobulin, sequence 2 | 1.0340784 | 1.1697701 | 1.6960365 | 2.055635 | 0.7209327 | 0.649959 | 0.7125688 |
| Matrix metalloproteinase-1 | 1.1912407 | 1.4351726 | 2.035382 | 1.5597044 | 1.68073 | 2.1468914 | 1.5582157 |
| Beta-tubulin, class I | 1.0230223 | 1.0003003 | 1.4682993 | 0.8410493 | 1.0758952 | 1.2991679 | 1.4226933 |
| Phase-1 RCT-161 | 0.9102384 | 0.9405335 | 0.903203 | 0.8483805 | 0.9394853 | 0.7614825 | 1.4949017 |
| 14-3-3 zeta | 1.5434755 | 1.7089969 | 2.2698455 | 2.2927547 | 1.1347823 | 1.4992849 | 0.9918877 |
| Stem cell factor | 0.7200591 | 0.7034744 | 0.5718657 | 0.5920138 | 0.8181506 | 0.6903132 | 0.7097413 |
| Macrophage inflammatory protein-2 alpha | 1.8937194 | 2.0735016 | 1.8752743 | 1.9107991 | 0.9728206 | 1.3845319 | 0.9662155 |
| Thymidylate synthase | 1.0174305 | 1.049172 | 1.0876154 | 1.0398474 | 1.0683302 | 0.9348688 | 0.9667164 |
| c-jun | 1.4034392 | 3.9854562 | 3.0143523 | 4.009134 | 0.9456323 | 1.4562079 | 2.2498882 |
| Superoxide dismutase Mn | 2.941786 | 8.289341 | 14.135337 | 17.289742 | 1.1861475 | 1.18179 | 1.0823966 |
| Phase-1 RCT-73 | 0.7926196 | 0.9005327 | 1.0843397 | 0.9995455 | 0.9181056 | 0.9447869 | 0.838891 |
| Macrophage inflammatory protein-1 alpha | 1.1345794 | 1.0463153 | 1.2573816 | 1.1030653 | 1.0180987 | 1.1414776 | 1.7150396 |
| Phase-1 RCT-214 | 0.8831326 | 0.8136953 | 0.5781573 | 0.6637799 | 1.0363982 | 1.0669336 | 0.8625286 |
| NADH-cytochrome b5 reductase | 0.4703055 | 0.7905841 | 0.4978043 | 0.3709405 | 0.8689585 | 0.590559 | 0.8149056 |
| Interleukin-1 beta | 1.2082739 | 1.2146724 | 1.4957434 | 1.2936944 | 1.5209517 | 1.868814 | 1.4461828 |
| NADP-dependent isocitrate | 0.3197886 | 0.7582756 | 0.276826 | 0.2174262 | 0.8994059 | 0.7530251 | 0.6116083 |
| dehydrogenase, cytosolic | | | | | | | |
| Phase-1 RCT-289 | 0.5567026 | 0.9430446 | 0.7342676 | 0.6408615 | 1.0281671 | 0.8338355 | 0.991003 |
| Glutamine synthetase | 2.2179005 | 2.5139816 | 3.0947185 | 3.1190624 | 0.6307512 | 0.8090729 | 0.8706603 |
| Phase-1 RCT-182 | 0.4477195 | 0.6341929 | 0.6077408 | 0.4917501 | 0.8022699 | 0.5102434 | 0.7889959 |
| Ubiquitin conjugating enzyme (RAD 6 | 1.2142897 | 1.5677243 | 2.0285778 | 2.098915 | 0.942265 | 0.9588838 | 0.6383004 |
| homologue) | | | | | | | |
| Extracellular-signal-regulated kinase 1 | 0.3001081 | 0.8117772 | 0.3510207 | 0.2713912 | 0.60866 | 0.3840543 | 1.0261567 |
| Emerin | 0.8713257 | 0.9772813 | 1.0578102 | 0.8819869 | 1.2181295 | 1.1767237 | 0.8343231 |
| Phase-1 RCT-78 | 0.8334091 | 0.9409019 | 0.7960627 | 0.7415028 | 1.0020458 | 0.745707 | 0.9129555 |
| Phase-1 RCT-212 | 0.9327826 | 0.8622806 | 0.8836635 | 0.8862546 | 0.9573777 | 0.865335 | 1.0099642 |
| Phase-1 RCT-168 | 0.5341207 | 0.770514 | 0.5934889 | 0.3465456 | 0.9360334 | 0.7828058 | 0.646455 |
| Lysyl hydroxylase | 1.6817493 | 1.4336053 | 1.4445691 | 1.5043573 | 0.7901776 | 0.7778358 | 0.9108456 |
| Phase-1 RCT-55 | 1.3323337 | 1.0170738 | 1.1765746 | 1.3183376 | 0.972007 | 0.9817303 | 1.5679711 |
| MHC class I antigen RT1.A1(f) alpha- | 1.4238466 | 1.9526731 | 1.8834743 | 2.6017783 | 1.2590507 | 1.8106252 | 1.6783357 |
| chain | | | | | | | |
| Phase-1 RCT-40 | 0.6441833 | 0.9509122 | 0.9162991 | 0.8891984 | 0.8249916 | 0.7191528 | 0.6595402 |
| Cyclin G | 2.1775928 | 3.4404607 | 7.979724 | 7.341203 | 1.2998382 | 0.862676 | 1.4873523 |
| Peroxisome proliferator activated receptor | 1 | 1.0215765 | 1.009515 | 0.9727257 | 1.0453944 | 1.0425767 | 1.1809654 |
| gamma | | | | | | | |
| Phase-1 RCT-280 | 0.7275981 | 0.8733755 | 0.7540843 | 0.861401 | 0.8549527 | 1.007301 | 0.8703985 |
| D-dopachrome tautomerase | 0.5841223 | 0.6413037 | 0.6597097 | 0.5320147 | 0.6931726 | 0.5649967 | 0.9256513 |
| Phase-1 RCT-287 | 0.828557 | 0.9359778 | 0.9963806 | 1.1918628 | 0.671769 | 0.7654696 | 0.625103 |
| Phase-1 RCT-119 | 1.2153145 | 0.8162751 | 1.1692823 | 0.6933279 | 1.1161451 | 0.8688476 | 0.9177563 |
| Thioredoxin-1 (Trx1) | 0.8671653 | 0.9459941 | 1.0023035 | 1.0982692 | 0.9099162 | 0.9990968 | 0.8360615 |
| Phase-1 RCT-225 | 1.4160423 | 0.6521165 | 0.5427963 | 0.8860172 | 1.2687068 | 0.9506828 | 1.1373031 |
| Protein kinase C alpha | 1.1121504 | 0.9189212 | 0.9921584 | 0.948838 | 0.8521274 | 0.9080062 | 1.010042 |
| Phase-1 RCT-87 | 0.9042152 | 1.0266783 | 0.9351091 | 1.0220834 | 0.799032 | 0.8384275 | 0.8356084 |
| Phase-1 RCT-59 | 1.9504247 | 2.6803663 | 3.4525228 | 3.8167098 | 0.9577852 | 0.912524 | 1.0679643 |
| C4b-binding protein | 0.7649313 | 1.1219428 | 1.285401 | 1.2177256 | 0.8936207 | 0.9056285 | 0.6300583 |
| Phase-1 RCT-204 | 2.257046 | 1.5538791 | 3.2973871 | 3.36897 | 0.9154698 | 0.8331398 | 1.2180467 |
| Carnitine palmitoyl-CoA transferase | 1.1330408 | 0.995792 | 0.9168253 | 1.1786226 | 0.9879704 | 2.1016593 | 1.1201242 |
| 8-oxoguanine DNA glycosylase | 1.350742 | 1.2064193 | 1.1115209 | 0.9493023 | 1.0469246 | 1.0056523 | 1.3216493 |
| H-rev107 | 1.1698531 | 1.1204135 | 1.0682408 | 0.9999999 | 0.9324258 | 0.8622732 | 0.9906792 |
| Preproalbumin | 0.6419438 | 0.862693 | 0.8034355 | 0.7905585 | 1.0962775 | 1.0494729 | 0.7094297 |
| Phase-1 RCT-177 | 0.6879447 | 0.8908346 | 0.9438881 | 1.0326767 | 0.8672021 | 0.9464925 | 0.681808 |
| Phase-1 RCT-110 | 1.0830106 | 0.9686695 | 0.9314909 | 0.9587038 | 0.9102574 | 0.9688659 | 1.2620728 |
| Phase-1 RCT-227 | 0.8079372 | 0.7195278 | 0.6383494 | 0.6926483 | 0.8467624 | 0.5918858 | 0.617042 |
| Neuropeptide Y | 0.9501234 | 1.296338 | 1.1445181 | 1.0936875 | 0.9732333 | 1.0192788 | 1.3269433 |
| Phase-1 RCT-277 | 0.5879663 | 0.8130282 | 0.8044767 | 0.65265 | 0.9719253 | 1.0040847 | 0.5241712 |
| Phase-1 RCT-229 | 1.0240241 | 0.9335889 | 0.8668065 | 0.8605851 | 1.0209498 | 0.9462986 | 1.2811127 |
| Phase-1 RCT-34 | 0.881638 | 0.6764618 | 0.6510202 | 0.6981545 | 1.0807405 | 1.044174 | 1.3272773 |
| Choline kinase | 1.1566594 | 1.4619123 | 1.2527808 | 1.5622853 | 0.9961212 | 0.9881606 | 1.4096638 |
| Phase-1 RCT-20 | 1.1315469 | 0.9623128 | 1.0858629 | 1.0565234 | 0.9482463 | 0.9319236 | 1.1789237 |
| Phase-1 RCT-248 | 1.0248734 | 0.955228 | 1.0154396 | 0.9969937 | 1.3358105 | 1.5456758 | 1.3494335 |
| | CPHOS | CPHOS | CPHOS | CYCA | CYCA | CYCA | |
| Compound-Dose (2) | 100 | 100 | 100 | 20 | 20 | 20 | |
| Animal Number (3) | 2151 | 2152 | 2153 | 421 | 422 | 423 | |
| Liver Toxicity Necrosis Classification (4) | no | no | no | no | no | no | |
| Gene Name (5) | | | | | | | |
| Gadd153 | 1.0537983 | 1.2465805 | 1.007254 | 1.443514 | 1.2364559 | 1.2442609 | |
| Interferon related developmental regulator | 1.1207484 | 0.900986 | 1.4844143 | 1.0364828 | 1.1643105 | 1.3609716 | |
| IFRD1 (PC4) | | | | | | | |
| ID-1 | 0.9822516 | 1.1492193 | 0.8987915 | 1.2017941 | 1.0905391 | 0.9930104 | |
| Insulin-like growth factor binding protein 1 | 2.4847174 | 1.2253555 | 2.5293207 | 1.3858792 | 1.5527766 | 1.5027355 | |
| NIPK | 1.1156201 | 1.1094444 | 1.2279603 | 1.0853899 | 0.9537697 | 0.8763466 | |
| Phase-1 RCT-50 | 1.0559547 | 0.9538594 | 1.1368606 | 1.1796147 | 0.964114 | 0.9935789 | |
| Sarcoplasmic reticulum calcium ATPase | 0.9893088 | 1.166248 | 1.009637 | 0.9408266 | 0.7624447 | 0.8329145 | |
| Phase-1 RCT-207 | 1.3795277 | 1.3000697 | 1.4803665 | 1.1154239 | 1.1237066 | 1.3242997 | |
| Acetyl-CoA carboxylase | 0.8337498 | 0.8834515 | 0.8416207 | 0.9767702 | 0.950153 | 0.9375011 | |
| Cathepsin L, sequence 2 | 2.175082 | 1.3696542 | 2.2591949 | 1.2628828 | 1.5413916 | 1.808275 | |
| Glucokinase | 0.479833 | 1.1305695 | 0.5890625 | 1.6630741 | 0.9114418 | 1.2507352 | |
| Gadd45 | 1.4900218 | 1.4199187 | 1.8735077 | 1.3334124 | 1.0859015 | 1.0681955 | |
| Phase-1 RCT-18 | 0.9022892 | 0.8273383 | 0.8771964 | 0.998203 | 0.8740392 | 0.8614071 | |
| 3-beta-hydroxysteroid dehydrogenase | 0.7348112 | 0.6745015 | 0.7038327 | 0.4880432 | 0.5953647 | 1.1849707 | |
| (HSD3B1) | | | | | | | |
| Phase-1 RCT-221 | 1.1229622 | 1.28848 | 1.2511262 | 1.0997255 | 1.3296378 | 1.3027854 | |
| Heme oxygenase | 1.0892235 | 1.5192341 | 2.1460571 | 1.2912079 | 1.7650267 | 1.7054217 | |
| Cyclin dependent kinase 4 | 1.0995982 | 1.1094725 | 1.0185672 | 1.3057233 | 1.5881155 | 1.1882174 | |
| Phase-1 RCT-75 | 1.0161562 | 1.1273503 | 1.095369 | 1.0168229 | 1.0215611 | 1.0415287 | |
| Phase-1 RCT-65 | 1.3491628 | 1.4387356 | 1.3280704 | 0.8850979 | 0.7892828 | 0.8739781 | |
| Cytochrome P450 1A1 | 1.0185412 | 1.2809564 | 0.9525853 | 1.4712571 | 0.6727427 | 0.6659737 | |
| Peroxisomal multifunctional enzyme type II | 0.8805089 | 0.6912038 | 0.9296644 | 1.0438043 | 1.1017718 | 1.0142903 | |
| DNA topoisomerase I | 0.7260371 | 0.674491 | 0.6649151 | 0.9285183 | 1.4486314 | 1.4512296 | |
| Focal adhesion kinase (pp125FAK) | 0.9175299 | 0.8831956 | 0.9239895 | 1.0660291 | 1.2521734 | 1.1561521 | |
| NGF-inducible anti-proliferative putative | 0.8182016 | 0.6689515 | 0.8768684 | 0.7221685 | 0.8937983 | 0.7766777 | |
| secreted protein (PC3) | | | | | | | |
| Phase-1 RCT-82 | 0.983355 | 1.0921866 | 0.8822463 | 0.961531 | 0.8937655 | 0.8864794 | |
| Senescence marker protein-30 | 0.3505011 | 0.3040859 | 0.3658148 | 0.7777901 | 0.8961045 | 0.6803365 | |
| Melanoma-associated antigen ME491 | 0.9707179 | 0.8676796 | 0.8841678 | 1.1790683 | 1.2803015 | 0.5605118 | |
| Activating transcription factor 3 | 1.4770133 | 1.4586717 | 1.0788481 | 0.9751461 | 1.0197465 | 1.2017199 | |
| Tryptophan hydroxylase | 0.758134 | 1.0477817 | 0.9742643 | 0.6806992 | 1.0130686 | 0.9704405 | |
| Zinc finger protein | 0.8739849 | 1.0571684 | 1.3479788 | 0.7466646 | 0.9020134 | 1.2223456 | |
| c-myc | 1.2305678 | 1.280541 | 1.1258637 | 1.1840752 | 0.8447694 | 1.0837215 | |
| Cholesterol 7-alpha- hydroxytase (P450 | 0.7788107 | 0.8876715 | 0.9350323 | 0.9215853 | 0.6708438 | 0.7167631 | |
| VII) | | | | | | | |
| Ferritin H-chain | 2.3920352 | 1.6393383 | 1.9972931 | 0.7563725 | 0.9198926 | 0.9576925 | |
| Phase-1 RCT-197 | 0.9506702 | 0.9876339 | 1.1544585 | 1.113861 | 1.1211852 | 1.2700075 | |
| Phase-1 RCT-179 | 0.8274637 | 0.7077777 | 1.008307 | 1.1079361 | 1.6419836 | 1.4292796 | |
| Phase-1 RCT-15 | 1.4216772 | 1.2252609 | 1.8802882 | 0.9749798 | 1.2182475 | 1.2366718 | |
| Argininosuccinate lyase | 1.4060395 | 1.4986552 | 2.9141905 | 0.8436613 | 0.8117246 | 0.9025778 | |
| Phase-1 RCT-49 | 1.0338758 | 1.082748 | 1.0952854 | 1.1106856 | 1.1849788 | 1.1604351 | |
| Carbonic anhydrase III | 0.1289246 | 0.2566063 | 0.1623811 | 0.4942864 | 0.1516015 | 0.3387672 | |
| Proliferating cell nuclear antigen gene | 1.1503438 | 1.2173884 | 1.0609454 | 1.0504204 | 0.8805561 | 0.8833866 | |
| Integrin beta1 | 2.066766 | 1.2066684 | 1.6846242 | 1.2449186 | 1.2937934 | 1.2828424 | |
| Caspase 6 | 1.346237 | 1.1530007 | 1.2345183 | 1.1349472 | 1.0082724 | 0.9897853 | |
| Phase-1 RCT-116 | 1.5890355 | 1.6828046 | 2.5646858 | 1.334231 | 1.0260279 | 0.9354929 | |
| Phase-1 RCT-109 | 1.606749 | 1.1282215 | 1.6901292 | 1.4259655 | 1.8186828 | 1.6651722 | |
| Phase-1 RCT-71 | 1.1053451 | 1.2155167 | 1.1119555 | 1.1681427 | 1.1653315 | 1.1954005 | |
| Elongation factor-1 alpha | 1.0409058 | 0.9001384 | 1.1790899 | 1.0570681 | 1.2862569 | 1.2006648 | |
| Phase-1 RCT-169 | 1.0017965 | 1.2806185 | 0.9413649 | 0.9545679 | 0.754987 | 0.844219 | |
| Pyruvate kinase, muscle | 1.2724626 | 1.1970702 | 1.5225846 | 1.2466319 | 1.1463708 | 1.1872618 | |
| Nucleosome assembly protein | 0.5295967 | 0.733227 | 0.9041942 | 0.801837 | 0.6231393 | 0.6522236 | |
| Phase-1 RCT-127 | 1.0408914 | 1.003346 | 1.0392346 | 0.9632683 | 1.3328681 | 1.4026546 | |
| Iron-responsive element-binding protein | 0.9166442 | 0.8242433 | 0.6962215 | 0.8982735 | 0.9378396 | 0.7964942 | |
| Phase-1 RCT-72 | 0.9042122 | 1.2450476 | 0.9836636 | 0.930736 | 0.871094 | 0.8856897 | |
| Phase-1 RCT-144 | 1.0562986 | 1.0606049 | 1.0546496 | 1.3602147 | 1.5189047 | 1.2960958 | |
| Phase-1 RCT-242 | 1.2582949 | 1.2363948 | 1.2231609 | 0.9339557 | 0.9475504 | 0.9666293 | |
| 17-beta hydroxysteroid dehydrogenase, | 0.2731903 | 0.2900973 | 0.6398263 | 0.5778986 | 0.2714085 | 0.2342754 | |
| type 2 | | | | | | | |
| Phase-1 RCT-70 | 1.2030113 | 1.3140118 | 1.1899722 | 1.166231 | 1.0906293 | 0.8753531 | |
| Ribosomal protein L13A | 2.020708 | 1.1460273 | 1.6821706 | 1.5412277 | 1.8160518 | 1.7179817 | |
| Cytochrome P450 2E1 | 0.584384 | 0.5954781 | 0.8424572 | 0.8442712 | 0.862747 | 0.9585818 | |
| AT-3 | 0.9394153 | 0.8521118 | 0.9398697 | 1.0731682 | 1.1536281 | 1.0453157 | |
| Phase-1 RCT-270 | 0.6639934 | 0.8741179 | 0.6351779 | 0.510878 | 0.4737396 | 0.6536524 | |
| Phase-1 RCT-123 | 0.9223173 | 0.8331618 | 0.8485045 | 1.0332271 | 0.9277999 | 0.946074 | |
| Alpha-2-macroglobulin, sequence 2 | 0.6843499 | 0.7752912 | 0.9570001 | 0.9099162 | 0.8879998 | 1 | |
| Matrix metalloproteinase-1 | 2.2537587 | 1.7290292 | 1.8657224 | 0.8376041 | 0.953397 | 0.9889938 | |
| Beta-tubulin, class I | 1.0363526 | 1.0280998 | 0.9383087 | 1.0093101 | 1.4135209 | 0.9034575 | |
| Phase-1 RCT-161 | 0.6145361 | 0.6551154 | 0.7430937 | 0.6540843 | 0.4121313 | 0.5094573 | |
| 14-3-3 zeta | 1.3561543 | 1.2397512 | 1.4723117 | 1.0915642 | 1.0659643 | 1.0202637 | |
| Stem cell factor | 0.4566778 | 0.5466166 | 0.5858542 | 0.8795533 | 0.9902129 | 0.7905756 | |
| Macrophage inflammatory protein-2 alpha | 1.8975666 | 1.4923637 | 1.81745 | 1.1189542 | 1.6698467 | 1.3748304 | |
| Thymidylate synthase | 1.0370343 | 0.994744 | 1.0224415 | 1.0200262 | 0.8720166 | 0.8695486 | |
| c-jun | 1.5425799 | 1.8548172 | 2.210535 | 1.1986566 | 1.0961754 | 1.1306467 | |
| Superoxide dismutase Mn | 1.2765156 | 1.0854034 | 1.2015598 | 1.3657142 | 1.5930569 | 1.8564385 | |
| Phase-1 RCT-73 | 0.8918687 | 0.688558 | 0.9189046 | 0.9932917 | 0.8367233 | 0.8951778 | |
| Macrophage inflammatory protein-1 alpha | 1.0700101 | 1.3797919 | 1.0195752 | 1.0115398 | 0.7866281 | 0.8223891 | |
| Phase-1 RCT-214 | 0.7405044 | 0.7018666 | 0.6654378 | 0.895938 | 0.9440229 | 0.8855812 | |
| NADH-cytochrome b5 reductase | 0.6009278 | 0.7507146 | 0.5280331 | 0.6774212 | 0.7847068 | 0.853794 | |
| Interleukin-1 beta | 2.0374193 | 1.5717815 | 1.5758362 | 0.8990706 | 0.9854895 | 1.0185239 | |
| NADP-dependent isocitrate | 0.6067706 | 0.6340609 | 0.5102063 | 0.6779712 | 0.8631481 | 0.6888476 | |
| dehydrogenase, cytosolic | | | | | | | |
| Phase-1 RCT-289 | 0.8534488 | 0.7687818 | 0.8071061 | 0.7652034 | 0.9160388 | 0.9959451 | |
| Glutamine synthetase | 0.4758248 | 0.6574614 | 0.8403165 | 1.1767477 | 1.3342221 | 1.4024062 | |
| Phase-1 RCT-182 | 0.6101838 | 0.8576264 | 0.5828691 | 0.6889819 | 0.6413258 | 0.9046662 | |
| Ubiquitin conjugating enzyme (RAD 6 | 0.992878 | 0.9097136 | 1.0861177 | 1.0265228 | 1.6217974 | 1.4206741 | |
| homologue) | | | | | | | |
| Extracellular-signal-regulated kinase 1 | 0.1917026 | 0.3320151 | 0.2912506 | 0.5924933 | 0.2081787 | 0.3966684 | |
| Emerin | 1.0761061 | 1.0195053 | 1.05902 | 1.0460086 | 1.0233744 | 0.9121569 | |
| Phase-1 RCT-78 | 0.7852229 | 0.9055641 | 0.8617407 | 0.8731573 | 0.6637717 | 0.7826992 | |
| Phase-1 RCT-212 | 0.7554106 | 0.845422 | 0.9046709 | 1.024717 | 1.0452812 | 1.0314667 | |
| Phase-1 RCT-168 | 0.7139158 | 0.599202 | 0.5144773 | 0.8373241 | 0.8988438 | 0.9681823 | |
| Lysyl hydroxylase | 0.6067131 | 0.4256047 | 0.8233594 | 1.1819198 | 1.3311744 | 1.4157789 | |
| Phase-1 RCT-55 | 0.9930617 | 1.2771955 | 1.1327554 | 0.9097688 | 0.9401507 | 1.0778093 | |
| MHC class I antigen RT1.A1(f) alpha- | 1.6321218 | 1.8973975 | 2.0893815 | 1.0904845 | 0.8449211 | 0.8958194 | |
| chain | | | | | | | |
| Phase-1 RCT-40 | 0.6625125 | 0.6895482 | 0.5673148 | 0.8237945 | 0.7740365 | 0.8344768 | |
| Cyclin G | 1.1576024 | 1.4068509 | 1.5097153 | 1.5629607 | 1.2649181 | 1.2373999 | |
| Peroxisome proliferator activated receptor | 1.0745851 | 1.0844343 | 1.0183909 | 0.8912673 | 0.9397514 | 1.2254982 | |
| gamma | | | | | | | |
| Phase-1 RCT-280 | 0.8787721 | 0.8471609 | 0.8787558 | 0.9135633 | 0.644517 | 0.8335333 | |
| D-dopachrome tautomerase | 0.6101964 | 0.809477 | 0.8099513 | 0.7137904 | 0.8595423 | 1.0034013 | |
| Phase-1 RCT-287 | 0.7938092 | 0.6993159 | 0.7828475 | 1.2399744 | 1.2551234 | 1.1686116 | |
| Phase-1 RCT-119 | 0.9998294 | 1.1084354 | 1.042591 | 0.5374739 | 0.9217802 | 1.0017986 | |
| Thioredoxin-1 (Trx1) | 0.993747 | 0.8653913 | 0.8606149 | 0.9930579 | 1.1818565 | 1.234687 | |
| Phase-1 RCT-225 | 0.796353 | 0.7849852 | 0.6127531 | 1.3494039 | 1.4222478 | 1.3610284 | |
| Protein kinase C alpha | 0.9955084 | 0.8517512 | 0.9262028 | 0.9921727 | 1.156413 | 1.0335637 | |
| Phase-1 RCT-87 | 0.8287216 | 0.9207022 | 0.9675403 | 0.9875783 | 1.0826209 | 1.1609998 | |
| Phase-1 RCT-59 | 1.1846848 | 1.2377837 | 1.5810862 | 1.0025473 | 1.1281986 | 1.0807378 | |
| C4b-binding protein | 0.6565414 | 0.5762771 | 0.6627492 | 1.0005138 | 1.3059664 | 1.3145951 | |
| Phase-1 RCT-204 | 0.8704483 | 1.0070261 | 0.9389781 | 1.0618204 | 1.0114563 | 1.086424 | |
| Carnitine palmitoyl-CoA transferase | 1.2882736 | 1.2846671 | 2.1152134 | 0.9368447 | 0.8186537 | 0.8162028 | |
| 8-oxoguanine DNA glycosylase | 1.0300897 | 0.9212371 | 0.9585341 | 1.0740162 | 1.0395677 | 0.9692444 | |
| H-rev107 | 0.8449257 | 0.8422323 | 0.825224 | 0.9103887 | 1.0997518 | 0.8523135 | |
| Preproalbumin | 0.993016 | 0.6824648 | 1.139516 | 0.7973324 | 0.7116489 | 0.8127667 | |
| Phase-1 RCT-177 | 0.8736814 | 0.7834948 | 0.8735638 | 0.9510196 | 0.834835 | 0.9441508 | |
| Phase-1 RCT-110 | 0.9530034 | 1.0327266 | 0.9518606 | 1.0211344 | 0.8280835 | 0.8492154 | |
| Phase-1 RCT-227 | 0.7521052 | 0.7423607 | 0.6124045 | 0.9153019 | 0.6645469 | 0.8119186 | |
| Neuropeptide Y | 0.987401 | 1.2345253 | 1.0196763 | 1.0213194 | 0.9999999 | 1.0373305 | |
| Phase-1 RCT-277 | 0.8011528 | 0.7220877 | 0.7867222 | 0.6062075 | 1.3159982 | 0.656356 | |
| Phase-1 RCT-229 | 0.9572815 | 1.1229413 | 1.0082563 | 0.9623197 | 0.7879899 | 0.8910313 | |
| Phase-1 RCT-34 | 1.028132 | 1.0646019 | 0.77224 | 0.7587988 | 0.700376 | 0.7006728 | |
| Choline kinase | 1.0687664 | 1.1541944 | 1.0556324 | 0.951274 | 0.8317233 | 0.9577274 | |
| Phase-1 RCT-20 | 0.9941705 | 1.1179793 | 1.0872451 | 1.0466816 | 0.8961483 | 0.9587385 | |
| Phase-1 RCT-248 | 1.5001171 | 1.1379092 | 1.1787723 | 1.2747824 | 1.080392 | 0.9997642 | |
| | CYCA | CYCA | CYCA | DEX | DEX | DEX | DEX |
| Compound-Dose (2) | 80 | 80 | 80 | 8 | 8 | 8 | 30 |
| Animal Number (3) | 431 | 432 | 433 | 1341 | 1342 | 1343 | 1351 |
| Liver Toxicity Necrosis Classification (4) | no | no | no | no | no | no | no |
| Gene Name (5) | | | | | | | |
| Gadd153 | 1.0448542 | 1.1062808 | 1.2771786 | 1.2676193 | 1.4142634 | 1.6155117 | 1.1178005 |
| Interferon related developmental regulator | 1.3141092 | 1.3790201 | 1.2410325 | 0.8615118 | 0.8937532 | 0.8694944 | 1.0676143 |
| IFRD1 (PC4) | | | | | | | |
| ID-1 | 1.029898 | 1.3377413 | 1.183672 | 1.0029364 | 1.0997725 | 1.0882101 | 0.8623245 |
| Insulin-like growth factor binding protein 1 | 1.6708524 | 1.975271 | 1.6783271 | 0.9581872 | 1.106588 | 1.1350757 | 1.0485095 |
| NIPK | 0.9903206 | 0.9619927 | 0.9074218 | 0.708931 | 0.7487857 | 0.8138657 | 0.77940311 |
| Phase-1 RCT-50 | 0.9356622 | 1.0353138 | 1.0311953 | 1.0012553 | 1.1225268 | 1.133866 | 1.1998295 |
| Sarcoplasmic reticulum calcium ATPase | 1.2492818 | 1.2282479 | 0.9599108 | 1.0001818 | 1.052906 | 1.1955922 | 1.2515774 |
| Phase-1 RCT-207 | 1.0974792 | 1.2349092 | 0.9861664 | 1.3837559 | 1.3576558 | 1.3743293 | 2.2989964 |
| Acetyl-CoA carboxylase | 1.0507295 | 1.0813622 | 1.0380164 | 1.0907692 | 0.9575013 | 0.9314078 | 1.0753624 |
| Cathepsin L, sequence 2 | 1.6041257 | 1.6025063 | 1.9211249 | 0.9383003 | 0.8948854 | 0.9191521 | 1.0548416 |
| Glucokinase | 0.997029 | 0.8238276 | 0.7480798 | 0.7225674 | 0.8764944 | 0.7566172 | 0.6425842 |
| Gadd45 | 1.0056016 | 1.2379446 | 1.1710308 | 2.0514271 | 3.5367138 | 2.8699548 | 2.092824 |
| Phase-1 RCT-18 | 0.8774132 | 0.9566197 | 0.8512021 | 0.9715411 | 0.9165813 | 0.8352406 | 0.8315123 |
| 3-beta-hydroxysteroid dehydrogenase | 1.2546198 | 0.9705944 | 1.0345578 | 0.9492603 | 0.8169687 | 0.7763053 | 0.9429565 |
| (HSD3B1) | | | | | | | |
| Phase-1 RCT-221 | 1.0526004 | 1.0601437 | 1.1534904 | 1.1095942 | 1.0538336 | 1.0784458 | 1.0041517 |
| Heme oxygenase | 1.1714281 | 1.4153603 | 1.5725106 | 0.7801099 | 0.7407051 | 0.9325072 | 0.8993611 |
| Cyclin dependent kinase 4 | 1.0884091 | 1.320963 | 1.4068112 | 1.0790273 | 1.0999094 | 1.095359 | 1.0942556 |
| Phase-1 RCT-75 | 1.0132331 | 1.0354784 | 0.9253702 | 1.0288439 | 1.0893854 | 1.0606279 | 0.8736609 |
| Phase-1 RCT-65 | 0.799988 | 0.8365154 | 0.8274743 | 1.0342023 | 1.1050642 | 1.1462489 | 1.3272464 |
| Cytochrome P450 1A1 | 0.8729764 | 0.9542013 | 0.6481403 | 0.8518818 | 0.9803349 | 0.831304 | 0.8913962 |
| Peroxisomal multifunctional enzyme type II | 1.1292479 | 1.0320228 | 1.1004341 | 1.1097003 | 1.0085999 | 1.026173 | 1.2851489 |
| DNA topoisomerase I | 1.1604326 | 1.2616745 | 1.2975012 | 0.8612108 | 0.9719967 | 0.9282814 | 0.7038006 |
| Focal adhesion kinase (pp125FAK) | 0.9804457 | 1.0626135 | 1.0201217 | 0.8539292 | 0.9412446 | 0.84095 | 0.8282015 |
| NGF-inducible anti-proliferative putative | 0.8392075 | 0.8357265 | 0.8228612 | 1.0475305 | 0.8079878 | 0.854571 | 0.8809096 |
| secreted protein (PC3) | | | | | | | |
| Phase-1 RCT-82 | 0.8419499 | 0.8468084 | 0.7814538 | 1.0922055 | 1.1130445 | 1.142924 | 0.938683 |
| Senescence marker protein-30 | 0.5626416 | 1.1559547 | 0.7006141 | 0.5275925 | 0.5153432 | 0.6271566 | 0.4328918 |
| Melanoma-associated antigen ME491 | 0.8976139 | 1.1357034 | 1.3080673 | 0.9766037 | 0.8934295 | 0.8713636 | 1.0901451 |
| Activating transcription factor 3 | 0.9811202 | 0.9654024 | 0.9266759 | 0.9532972 | 1.2260133 | 1.1333228 | 1.0412967 |
| Tryptophan hydroxylase | 0.8744866 | 0.8064646 | 0.8904945 | 0.730473 | 0.8443807 | 0.6650286 | 0.7685609 |
| Zinc finger protein | 1.0896904 | 0.8645428 | 0.9043073 | 0.8547971 | 0.8073008 | 0.8154201 | 1.0725034 |
| c-myc | 1.0686601 | 1.1365633 | 1.0419921 | 1.1998042 | 1.4852221 | 1.5397022 | 1.2892656 |
| Cholesterol 7-alpha-hydroxylase P450 VII) | 0.8200423 | 0.7330146 | 0.7024047 | 0.7875821 | 0.9425688 | 0.8550745 | 0.7105084 |
| Ferritin H-chain | 1.0956482 | 0.7133642 | 1.0063157 | 1.102701 | 1.0742872 | 0.9369315 | 0.9607744 |
| Phase-1 RCT-197 | 0.9971616 | 1.1335856 | 1.0945284 | 0.8987544 | 0.9054094 | 0.9073995 | 0.9847385 |
| Phase-1 RCT-179 | 1.7407955 | 1.9883666 | 1.7229999 | 0.9872915 | 0.951387 | 0.9325966 | 1.413844 |
| Phase-1 RCT-15 | 1.1572878 | 1.0976168 | 1.3326694 | 1.0463127 | 0.896919 | 0.899007 | 1.4741883 |
| Argininosuccinate lyase | 0.9207423 | 0.6909263 | 0.8176806 | 1.0564291 | 0.8346627 | 0.9890683 | 1.6101395 |
| Phase-1 RCT-49 | 0.9269975 | 1.236216 | 1.1589782 | 0.9014867 | 0.8526791 | 0.8725641 | 1.1160021 |
| Carbonic anhydrase III | 0.3477499 | 0.1476942 | 0.3266841 | 0.5732966 | 0.5964452 | 0.7135139 | 0.563068 |
| Proliferating cell nuclear antigen gene | 0.9836865 | 1.0815179 | 0.8121728 | 1.0752847 | 1.3066941 | 1.4247342 | 1.9005954 |
| Integrin beta1 | 1.1610616 | 1.3484857 | 1.4490657 | 1.3349885 | 1.3099735 | 1.4763737 | 1.2379732 |
| Caspase 6 | 1.0017357 | 0.9876387 | 1.0481861 | 1.1000617 | 1.4060401 | 1.3827395 | 1.1101452 |
| Phase-1 RCT-116 | 0.9950206 | 0.8826702 | 1.0719081 | 0.948579 | 1.032105 | 0.9339993 | 1.2026467 |
| Phase-1 RCT-109 | 1.2812439 | 1.8000199 | 1.734601 | 1.1542435 | 1.2114013 | 1.1230981 | 1.2200902 |
| Phase-1 RCT-71 | 1.0133089 | 1.170251 | 1.0406955 | 1.3495172 | 1.2126992 | 1.4044182 | 1.7031835 |
| Elongation factor-1 alpha | 1.2842697 | 1.3338126 | 1.2158965 | 1.0828885 | 1.1127689 | 0.9442936 | 1.0096111 |
| Phase-1 RCT-169 | 0.9421971 | 0.7816191 | 0.7981601 | 1.053975 | 1.1393448 | 1.1699146 | 1.0354978 |
| Pyruvate kinase, muscle | 1.047775 | 1.0649478 | 1.3313003 | 1.0765637 | 0.9614993 | 0.9330426 | 1.019729 |
| Nucleosome assembly protein | 0.8056495 | 0.7347529 | 0.6461248 | 0.7220354 | 0.687533 | 0.781491 | 0.7188268 |
| Phase-1 RCT-127 | 0.9953476 | 1.3650078 | 1.2906947 | 1.1783849 | 0.8647643 | 1.045848 | 0.911347 |
| Iron-responsive element-binding protein | 0.9259862 | 0.8299521 | 0.7738088 | 1.2816826 | 1.5969173 | 1.4640955 | 1.1725314 |
| Phase-1 RCT-72 | 0.882441 | 0.821406 | 0.8243071 | 1.1128618 | 1.2165186 | 1.224332 | 1.1047316 |
| Phase-1 RCT-144 | 1.21975 | 2.1631913 | 1.641503 | 1.2642703 | 1.1573822 | 1.2043765 | 1.7358122 |
| Phase-1 RCT-242 | 0.9184537 | 0.9891263 | 0.9770286 | 1.2481409 | 1.2973025 | 1.3919817 | 1.381328 |
| 17-beta hydroxysteroid dehydrogenase, | 0.4616346 | 0.3804571 | 0.2994705 | 0.6617636 | 0.6250637 | 0.7619933 | 0.6443807 |
| type 2 | | | | | | | |
| Phase-1 RCT-70 | 0.9020996 | 0.8516315 | 0.8589156 | 1.1202481 | 1.300387 | 1.2997338 | 0.8473226 |
| Ribosomal protein L13A | 2.3402135 | 2.235569 | 1.8361284 | 1.2827929 | 1.3618011 | 1.2714775 | 1.1614312 |
| Cytochrome P450 2E1 | 0.9210291 | 1.1101815 | 0.6958855 | 0.7820702 | 1.0882152 | 1.0545316 | 0.7129645 |
| AT-3 | 0.8983433 | 1.0104859 | 1.0461462 | 1.0248334 | 0.9232575 | 0.9262161 | 0.9349489 |
| Phase-1 RCT-270 | 0.5837159 | 0.4134402 | 0.4870429 | 1.3789811 | 1.1926533 | 1.226688 | 1.819257 |
| Phase-1 RCT-123 | 0.9454186 | 0.9404165 | 0.9357019 | 0.8762747 | 0.8933085 | 0.9628746 | 0.9966711 |
| Alpha-2-macroglobulin, sequence 2 | 1.2952836 | 1.1325079 | 1.0337226 | 1.0788192 | 1.0268149 | 1.1130042 | 1.6050067 |
| Matrix metalloproteinase-1 | 1.0782433 | 0.8595834 | 1.1006273 | 1.129776 | 1.0463583 | 0.9434875 | 0.9661403 |
| Beta-tubulin, class I | 0.9851387 | 1.1159232 | 0.7974778 | 1.0421367 | 0.8633926 | 0.9166709 | 1.3430926 |
| Phase-1 RCT-161 | 0.7814521 | 0.644448 | 0.6118631 | 1.0538521 | 0.9347342 | 1 | 0.7928577 |
| 14-3-3 zeta | 1.1445826 | 1.1136858 | 1.0505913 | 1.167013 | 1.3180228 | 1.445074 | 1.2844964 |
| Stem cell factor | 0.7453783 | 1.1172677 | 0.8319036 | 0.5029929 | 0.5178375 | 0.6315113 | 0.5488306 |
| Macrophage inflammatory protein-2 alpha | 1.5260947 | 2.3906202 | 2.145872 | 1.2837749 | 1.3842411 | 1.813666 | 1.3204743 |
| Thymidylate synthase | 1.080214 | 0.9775475 | 0.9296596 | 0.672777 | 0.8202269 | 0.7141326 | 0.6735051 |
| c-jun | 0.9616714 | 1.0766805 | 1.1322987 | 1.1203958 | 1.5576555 | 1.8653525 | 1.5094707 |
| Superoxide dismutase Mn | 1.6509271 | 3.5769284 | 2.359766 | 1.0498444 | 1.1783663 | 1.2190272 | 1.0162449 |
| Phase-1 RCT-73 | 0.8794668 | 0.8266149 | 0.9943274 | 0.9680381 | 0.9378202 | 0.858408 | 0.9493389 |
| Macrophage inflammatory protein-1 alpha | 1.0985497 | 0.851912 | 0.9754973 | 1.1755428 | 1.3509679 | 1.3521217 | 1.0310795 |
| Phase-1 RCT-214 | 0.975714 | 0.7642539 | 0.8336229 | 0.9206429 | 0.8071424 | 0.7919713 | 0.5956691 |
| NADH-cytochrome b5 reductase | 0.8982445 | 0.6814376 | 0.6929386 | 0.8102948 | 0.6703005 | 0.8403298 | 0.7068526 |
| Interleukin-1 beta | 1.025522 | 0.9060589 | 1.1454202 | 1.1197803 | 1.044138 | 0.9601806 | 0.9632663 |
| NADP-dependent isocitrate | 0.8170621 | 0.553668 | 0.684319 | 0.7626141 | 0.6396906 | 0.6665036 | 0.4897769 |
| dehydrogenase, cytosolic | | | | | | | |
| Phase-1 RCT-289 | 0.9622987 | 0.8333645 | 0.8965346 | 0.8108919 | 0.7268961 | 0.71125 | 0.9440518 |
| Glutamine synthetase | 1.7561529 | 1.3998247 | 1.3604645 | 1.1391616 | 1.167084 | 0.9279367 | 1.1994969 |
| Phase-1 RCT-182 | 0.8735136 | 0.6863826 | 0.8290766 | 0.9323641 | 0.9533215 | 0.9500009 | 0.8761675 |
| Ubiquitin conjugating enzyme (RAD 6 | 1.8214389 | 1.5250822 | 1.6920667 | 1.3024272 | 1.3462974 | 1.3540735 | 1.6043805 |
| homologue) | | | | | | | |
| Extracellular-signal-regulated kinase 1 | 0.6233946 | 0.9625297 | 0.5173074 | 0.572185 | 0.5810977 | 0.7431218 | 0.5950753 |
| Emerin | 1.013781 | 1 | 0.8785525 | 0.9509556 | 0.9745811 | 1.0351745 | 1.1860683 |
| Phase-1 RCT-78 | 0.9312837 | 0.779525 | 0.794976 | 0.9040387 | 0.83184 | 0.8732051 | 0.8317867 |
| Phase-1 RCT-212 | 0.9693106 | 0.9231517 | 0.9603477 | 0.9994264 | 0.965355 | 0.8363291 | 1.0638953 |
| Phase-1 RCT-168 | 0.9305897 | 0.8179372 | 0.9493972 | 0.8540679 | 0.7876226 | 0.7621049 | 0.60016 |
| Lysyl hydroxylase | 1.1780622 | 1.2416638 | 1.3468019 | 1.0988544 | 1.1340454 | 0.9403684 | 1.0662886 |
| Phase-1 RCT-55 | 0.9223744 | 1.0828516 | 0.875153 | 1.3644245 | 1.3198231 | 1.265233 | 1.2543198 |
| MHC class I antigen RT1.A1(f) alpha- | 0.9825864 | 0.9571455 | 0.9822382 | 0.9329866 | 1.115608 | 1.17681 | 0.9278817 |
| chain | | | | | | | |
| Phase-1 RCT-40 | 0.8212654 | 0.6567124 | 0.7676137 | 0.9464196 | 0.8992323 | 0.7824384 | 0.5945833 |
| Cyclin G | 1.1067655 | 1.333024 | 1.5311562 | 1.8598254 | 1.4008944 | 2.0356812 | 1.6186402 |
| Peroxisome proliferator activated receptor | 1.0130031 | 1.0372584 | 1.1668606 | 0.6861407 | 0.8551145 | 0.7438429 | 0.5281569 |
| gamma | | | | | | | |
| Phase-1 RCT-280 | 1.0113076 | 0.7665545 | 0.8998672 | 0.924785 | 0.8115763 | 0.7961428 | 0.98528 |
| D-dopachrome tautomerase | 0.8615303 | 0.682361 | 0.8440793 | 0.9117773 | 0.8615373 | 0.7807652 | 0.8403492 |
| Phase-1 RCT-287 | 0.9747023 | 1.0218469 | 1.15959 | 0.9399977 | 0.87089 | 0.9058751 | 0.8755269 |
| Phase-1 RCT-119 | 0.9807099 | 0.821719 | 0.7841557 | 0.8754526 | 1.1518507 | 1.1723303 | 0.9768366 |
| Thioredoxin-1 (Trx1) | 1.1662415 | 1.3173422 | 1.3170868 | 0.8563615 | 0.8344261 | 0.8634213 | 0.8604289 |
| Phase-1 RCT-225 | 0.802034 | 0.794386 | 0.9515151 | 1.0830187 | 1.0209783 | 0.8553363 | 0.9463546 |
| Protein kinase C alpha | 0.9683781 | 1.0531288 | 1.120766 | 1.1516237 | 1.0240799 | 1.0796145 | 1.1504664 |
| Phase-1 RCT-87 | 0.968205 | 1.0850959 | 1.0856353 | 1.157416 | 1.176778 | 1.1766019 | 0.9344432 |
| Phase-1 RCT-59 | 1.1163602 | 1.145961 | 0.8559114 | 1.137144 | 1.2653494 | 1.1582404 | 1.620731 |
| C4b-binding protein | 1.1054826 | 1.0395092 | 1.171393 | 0.7774158 | 0.734317 | 0.747693 | 0.5953353 |
| Phase-1 RCT-204 | 0.9266055 | 0.9498541 | 0.94058 | 0.8442035 | 0.8281834 | 0.9292849 | 0.9456191 |
| Carnitine palmitoyl-CoA transferase | 1.0092452 | 1.1090086 | 0.8311551 | 1.0542902 | 1.0681357 | 1.3321323 | 1.7088845 |
| 8-oxoguanine DNA glycosytase | 0.998879 | 1.0761906 | 0.9963116 | 0.8419015 | 0.9153228 | 0.8821058 | 0.9225683 |
| H-rev107 | 0.9053375 | 0.9859265 | 0.7357439 | 0.7936894 | 0.8255988 | 0.7826744 | 0.8201742 |
| Preproalbumin | 0.956306 | 0.5243141 | 0.9553706 | 0.9849722 | 0.8919662 | 0.7891854 | 1.0414095 |
| Phase-1 RCT-177 | 0.9538607 | 0.8263714 | 1.0505606 | 0.9439296 | 0.9134363 | 0.8463976 | 0.9029324 |
| Phase-1 RCT-110 | 0.9472396 | 0.8999551 | 0.8155932 | 0.9594933 | 0.7825413 | 0.8849815 | 0.8914391 |
| Phase-1 RCT-227 | 0.8906134 | 0.6739017 | 0.8519733 | 0.9996111 | 0.8956006 | 0.9464451 | 0.8428918 |
| Neuropeptide Y | 0.9022313 | 1.067271 | 0.9395639 | 0.9775518 | 0.8616969 | 0.9312139 | 0.9539022 |
| Phase-1 RCT-277 | 1.0019972 | 0.7895345 | 0.7717893 | 0.4827275 | 0.5085256 | 0.3465772 | 0.4308463 |
| Phase-1 RCT-229 | 0.8727707 | 0.8997638 | 0.8299456 | 0.9217982 | 0.8900046 | 0.9336107 | 0.9152765 |
| Phase-1 RCT-34 | 0.7539919 | 0.7629598 | 0.6622958 | 0.9864492 | 1.0880926 | 1.1605731 | 1.0266217 |
| Choline kinase | 0.9426768 | 1.0673144 | 0.9042736 | 0.9476454 | 0.8707264 | 1.077104 | 1.0917796 |
| Phase-1 RCT-20 | 0.8281602 | 0.8453699 | 0.8510186 | 1.1546807 | 1.018189 | 1.1944602 | 1.2448725 |
| Phase-1 RCT-248 | 0.8897792 | 1.1104382 | 1.2257625 | 1.2305909 | 1.2822331 | 1.2438203 | 1.1301842 |
| | DEX | DEX | DIF | DIF | DIF | DIF | DIF |
| Compound-Dose (2) | 30 | 30 | 25 | 25 | 25 | 100 | 100 |
| Animal Number (3) | 1352 | 1353 | 241 | 242 | 243 | 251 | 252 |
| Liver Toxicity Necrosis Classification (4) | no | no | no | no | no | no | no |
| Gene Name (5) | | | | | | | |
| Gadd153 | 1.3884002 | 1.4505817 | 1.3836128 | 1.3886435 | 1.2758768 | 1.2714417 | 0.8517901 |
| Interferon related developmental regulator | 0.8943749 | 0.930337 | 1.1698748 | 0.7850649 | 0.992474 | 0.8120433 | 1.0738144 |
| IFRD1 (PC4) | | | | | | | |
| ID-1 | 1.0639237 | 1.085857 | 1.2509584 | 1.267821 | 1.3549134 | 0.839346 | 0.9179503 |
| Insulin-like growth factor binding protein 1 | 0.9751497 | 1.2947624 | 0.7400774 | 0.6369238 | 0.5929802 | 0.860744 | 0.6078306 |
| NIPK | 0.8852137 | 0.5622988 | 1.0083054 | 0.9015347 | 0.769694 | 0.5354585 | 0.7408367 |
| Phase-1 RCT-50 | 1.3079531 | 1.1411738 | 1.3499454 | 1.3055284 | 1.1657332 | 1.2993397 | 1.1766099 |
| Sarcoplasmic reticulum calcium ATPase | 1.1613208 | 1.1398766 | 0.7185942 | 0.8378656 | 0.74305 | 0.5695874 | 0.7126101 |
| Phase-1 RCT-207 | 1.8582406 | 1.6087422 | 0.9766804 | 0.967289 | 0.9630512 | 1.0655282 | 1.0652405 |
| Acetyl-CoA carboxylase | 1.0878452 | 1.1677774 | 1.041591 | 0.9699795 | 1.0282824 | 1.2019682 | 1.1059489 |
| Cathepsin L, sequence 2 | 1.0639824 | 1.0744483 | 1.3068584 | 1.0927022 | 1.598235 | 1.2622269 | 1.074526 |
| Glucokinase | 0.8671283 | 0.8320906 | 1.1187791 | 1.3953642 | 1.1708927 | 1.4098628 | 0.7664111 |
| Gadd45 | 1.7448965 | 1.8658012 | 1.4027362 | 1.1639798 | 1.1474477 | 0.9952292 | 0.9214307 |
| Phase-1 RCT-18 | 0.892825 | 0.8900819 | 0.8773513 | 0.9031503 | 0.9332957 | 0.9856342 | 0.9959967 |
| 3-beta-hydroxysteroid dehydrogenase | 0.8351318 | 0.9063346 | 1.0077075 | 0.6072537 | 1.0339527 | 2.0005724 | 1.3610573 |
| (HSD3B1) | | | | | | | |
| Phase-1 RCT-221 | 1.1080614 | 1.1173856 | 0.77669 | 0.8083066 | 0.8545653 | 0.7586259 | 0.8183469 |
| Heme oxygenase | 0.8520559 | 1.0894725 | 1.5041407 | 0.9269143 | 1.1719396 | 0.9458494 | 1.4612912 |
| Cyclin dependent kinase 4 | 1.0487453 | 1.1181413 | 1.8922783 | 1.4025822 | 1.5556413 | 2.9893353 | 1.333256 |
| Phase-1 RCT-75 | 1.023999 | 0.9693367 | 1.1268014 | 1.116383 | 1.0740708 | 1.5082092 | 1.0836552 |
| Phase-1 RCT-65 | 1.4315622 | 1.0502143 | 1.2196866 | 1.3931428 | 1.1058694 | 1.1793308 | 1.0538352 |
| Cytochrome P450 1A1 | 1.0608116 | 1.0316557 | 0.999652 | 0.8746811 | 1.0536481 | 0.7374761 | 1.0045917 |
| Peroxisomal multifunctional enzyme type II | 1.0277601 | 1.1628624 | 0.9949061 | 1.352535 | 1.2344862 | 1.836753 | 1.4250157 |
| DNA topoisomerase I | 0.7378568 | 0.9114692 | 0.918578 | 0.7266573 | 0.7973736 | 0.9260368 | 0.8689088 |
| Focal adhesion kinase (pp125FAK) | 0.8159593 | 0.8470408 | 0.8809897 | 0.7685088 | 0.7611753 | 0.3541577 | 0.7950546 |
| NGF-inducible anti-proliferative putative | 0.9175451 | 0.8719331 | 0.8334383 | 0.8039421 | 0.63283 | 0.6364878 | 0.8026792 |
| secreted protein (PC3) | | | | | | | |
| Phase-1 RCT-82 | 1.045203 | 0.9969599 | 0.7907858 | 0.8290001 | 0.8425973 | 0.870289 | 0.9296427 |
| Senescence marker protein-30 | 0.4013995 | 0.3287787 | 0.6928645 | 0.8946214 | 0.73067 | 0.9923416 | 0.5960037 |
| Melanoma-associated antigen ME491 | 0.9841162 | 0.9561136 | 0.8534896 | 1.290025 | 0.880567 | 1.1540784 | 1.1918983 |
| Activating transcription factor 3 | 1.0748057 | 0.9421904 | 1.2269067 | 1.4822936 | 1.3552039 | 2.243396 | 1.4424229 |
| Tryptophan hydroxylase | 0.7086304 | 0.6489818 | 1.2861114 | 1.1873051 | 1.2228482 | 2.5557704 | 1.0591683 |
| Zinc finger protein | 0.9140933 | 0.7835156 | 1.017046 | 1.0859337 | 0.8406397 | 1.1405156 | 1.0949991 |
| c-myc | 1.7051003 | 1.7871655 | 1.5070794 | 1.5667894 | 1.5549897 | 1.2679186 | 1.0942013 |
| Cholesterol 7-alpha-hydroxylase P450 VII) | 0.8881279 | 0.8076834 | 1.2069831 | 0.7974609 | 0.9589351 | 0.6997881 | 0.878829 |
| Ferritin H-chain | 0.9933892 | 1.0881922 | 0.7006284 | 0.7452266 | 0.7640601 | 1.0190778 | 0.702664 |
| Phase-1 RCT-197 | 0.8579074 | 0.9470594 | 0.9726237 | 0.8744733 | 0.884077 | 0.8188993 | 1.057327 |
| Phase-1 RCT-179 | 1.3673564 | 1.2220016 | 0.9853198 | 0.8566204 | 0.8705891 | 1.0240241 | 0.8715376 |
| Phase-1 RCT-15 | 1.3337117 | 1.019709 | 1.7794156 | 1.8029552 | 1.7932677 | 6.787993 | 2.6882167 |
| Argininosuccinate lyase | 1.3793374 | 1.0466518 | 1.5252221 | 1.461911 | 1.569452 | 1.6442823 | 1.3705016 |
| Phase-1 RCT-49 | 1.0640091 | 0.9228432 | 0.8975433 | 0.8570516 | 0.7597046 | 0.9174421 | 1.0353292 |
| Carbonic anhydrase III | 0.5366234 | 0.3332017 | 0.5133709 | 1.4482896 | 1.4290621 | 4.6755233 | 0.5117113 |
| Proliferating cell nuclear antigen gene | 1.2650155 | 1.3149804 | 1.1186911 | 0.9438652 | 0.8869636 | 0.6060299 | 0.7398226 |
| Integrin beta1 | 1.2603636 | 1.5112165 | 1.0167036 | 0.8384475 | 0.8056949 | 1.3058773 | 1.0266104 |
| Caspase 6 | 1.1449169 | 1.2995298 | 1.237789 | 1.1723615 | 1.0759863 | 1.4007044 | 1.1605276 |
| Phase-1 RCT-116 | 1.2620486 | 1.2628659 | 3.7507155 | 4.0536056 | 4.9414487 | 2.0624855 | 1.3880414 |
| Phase-1 RCT-109 | 1.5402095 | 1.400646 | 0.6136423 | 0.6791973 | 0.5786843 | 0.5758885 | 0.6740695 |
| Phase-1 RCT-71 | 1.9540204 | 1.5232381 | 0.8619935 | 0.8287436 | 0.9671632 | 0.5125247 | 0.8398779 |
| Elongation factor-1 alpha | 1.069449 | 1.1323607 | 1.1356872 | 1.1188382 | 1.2151353 | 1.5381154 | 0.9617647 |
| Phase-1 RCT-169 | 1.1122804 | 1.1024202 | 0.7128326 | 0.932589 | 0.8971795 | 0.8633513 | 0.7696306 |
| Pyruvate kinase, muscle | 1.1153003 | 1.1036066 | 1.1439568 | 1.1104981 | 1.1888062 | 1.3816341 | 1.1972934 |
| Nucleosome assembly protein | 0.7463301 | 1.107617 | 1.1195881 | 0.7234067 | 0.90957 | 0.7380998 | 2.1552904 |
| Phase-1 RCT-127 | 0.8838567 | 0.8191499 | 1.0961393 | 0.9309346 | 0.9256746 | 0.7498946 | 1.0683295 |
| Iron-responsive element-binding protein | 1.1640453 | 1.6355906 | 1.0352157 | 1.0799178 | 1.1832844 | 0.7847299 | 1.1222669 |
| Phase-1 RCT-72 | 1.1632466 | 1.1491065 | 0.9404566 | 1.0798408 | 1.0295455 | 1.0164621 | 0.9139234 |
| Phase-1 RCT-144 | 1.4481487 | 1.5749441 | 0.9593955 | 0.9278081 | 0.8853776 | 1.1983722 | 1.0654476 |
| Phase-1 RCT-242 | 1.4221622 | 1.2410867 | 0.9835621 | 1.1220657 | 1.0486945 | 1.1194212 | 1.0453906 |
| 17-beta hydroxysteroid dehydrogenase, | 0.69302 | 1.0982496 | 1.1633836 | 0.578316 | 0.7876908 | 0.6482649 | 1.9994202 |
| type 2 | | | | | | | |
| Phase-1 RCT-70 | 1.0446771 | 0.9381395 | 0.9523955 | 0.9804366 | 0.9450029 | 0.9195268 | 0.940455 |
| Ribosomal protein L13A | 1.5147691 | 1.626859 | 1.0750962 | 0.9502932 | 0.7166286 | 0.561684 | 0.5661899 |
| Cytochrome P450 2E1 | 0.7163754 | 0.8269439 | 0.9114155 | 0.8971928 | 1.082249 | 1.4985788 | 1.208799 |
| AT-3 | 0.9060373 | 0.9031281 | 0.9976614 | 1.0689057 | 0.9859455 | 1.1461328 | 1.076572 |
| Phase-1 RCT-270 | 1.4584994 | 1.5315052 | 0.6152946 | 0.7676816 | 0.7231596 | 0.8639491 | 0.8048289 |
| Phase-1 RCT-123 | 0.9797359 | 0.9073246 | 1.1024798 | 1.114093 | 1.0483193 | 0.9419664 | 0.9598299 |
| Alpha-2-macroglobulin, sequence 2 | 1.4508644 | 0.9458726 | 0.7219253 | 0.6696338 | 0.610657 | 0.3906158 | 0.690736 |
| Matrix metalloproteinase-1 | 1.0331739 | 1.1545876 | 0.7825577 | 0.7912079 | 0.8154131 | 1.0439895 | 0.7853538 |
| Beta-tubulin, class I | 1.2449441 | 1.2044665 | 1.2288083 | 2.543356 | 1.6929046 | 1.181457 | 1.1384543 |
| Phase-1 RCT-161 | 0.9201918 | 0.9120823 | 1.1744668 | 2.0702195 | 1.6858728 | 1.2710149 | 0.9750736 |
| 14-3-3 zeta | 1.3499728 | 1.3743372 | 1.2006785 | 1.1865026 | 1.139614 | 2.060828 | 1.1337401 |
| Stem cell factor | 0.5445715 | 0.4089551 | 0.9682111 | 1.0397775 | 0.896601 | 1.1964816 | 0.6607523 |
| Macrophage inflammatory protein-2 alpha | 1.3485932 | 1.3360571 | 1.0865799 | 1.2007248 | 1.3977265 | 0.8616945 | 0.8197633 |
| Thymidylate synthase | 0.764994 | 0.6170235 | 1.2070657 | 1.12871 | 0.9659622 | 0.7437973 | 1.2124983 |
| c-jun | 1.585089 | 1.4558537 | 1.1943449 | 1.2137611 | 1.2389246 | 1.1407274 | 1.0762353 |
| Superoxide dismutase Mn | 1.1414894 | 1.0854832 | 1.2079935 | 1.2678173 | 1.2752644 | 1.4448665 | 0.9747192 |
| Phase-1 RCT-73 | 0.8650031 | 0.938485 | 1.140803 | 1.1397561 | 1.2475387 | 1.691877 | 1.1438065 |
| Macrophage inflammatory protein-1 alpha | 1.1670322 | 1.2244252 | 1.3512604 | 1.7061254 | 1.3576747 | 1.1855714 | 1.1152385 |
| Phase-1 RCT-214 | 0.6044087 | 0.7577235 | 1.521052 | 1.4387845 | 1.3272336 | 1.3554646 | 1.3219737 |
| NADH-cytochrome b5 reductase | 0.7087764 | 0.6204653 | 0.8140621 | 0.9607474 | 0.9041074 | 1.060259 | 0.9536001 |
| Interleukin-1 beta | 1.0268818 | 1.1875945 | 0.7463105 | 0.778241 | 0.8020974 | 1.0211571 | 0.8422411 |
| NADP-dependent isocitrate | 0.5789322 | 0.6172546 | 1.0427985 | 0.9471531 | 0.9471216 | 0.9001811 | 1.0730528 |
| dehydrogenase, cytosolic | | | | | | | |
| Phase-1 RCT-289 | 0.8025556 | 0.8754532 | 0.7282928 | 0.9721758 | 0.8867394 | 0.964187 | 1.0445987 |
| Glutamine synthetase | 1.0126511 | 1.4310333 | 0.9138616 | 0.9992308 | 1.0646193 | 1.0654869 | 0.997672 |
| Phase-1 RCT-182 | 0.8179189 | 0.8951438 | 0.670976 | 0.8547485 | 0.8546137 | 0.9064102 | 0.9435321 |
| Ubiquitin conjugating enzyme (RAD 6 | 1.3621775 | 1.5021722 | 0.8515219 | 0.9730741 | 0.9011802 | 1.1470127 | 0.9234148 |
| homologue) | | | | | | | |
| Extracellular-signal-regulated kinase 1 | 0.5922263 | 0.3807094 | 0.9464419 | 1.5064771 | 1.49592 | 3.9622078 | 0.5163713 |
| Emerin | 1.0215691 | 1.075294 | 1.0607293 | 1.1090199 | 1.0478067 | 1.0093412 | 1.1519376 |
| Phase-1 RCT-78 | 0.8841131 | 0.7867037 | 0.9243317 | 1.1862726 | 1.1662916 | 1.35.14017 | 1.0214039 |
| Phase-1 RCT-212 | 1.1462754 | 1.2530912 | 1.0377884 | 1.0269414 | 0.9496968 | 0.8385461 | 0.6587467 |
| Phase-1 RCT-168 | 0.7539933 | 0.7682384 | 0.8391526 | 0.9078428 | 0.8521743 | 0.9000326 | 1.1230217 |
| Lysyl hydroxylase | 0.9325329 | 1.228128 | 0.8460374 | 0.879957 | 0.9423906 | 1.0896537 | 1.0469593 |
| Phase-1 RCT-55 | 2.097427 | 2.2929475 | 0.6253163 | 1.2662809 | 0.948884 | 1.1010554 | 0.9187591 |
| MHC class I antigen RT1.A1(f) alpha- | 0.9972669 | 1.0193107 | 1.1361642 | 1.3759997 | 1.0705094 | 1.0099671 | 1.2194107 |
| chain | | | | | | | |
| Phase-1 RCT-40 | 0.7221244 | 0.8075842 | 0.9848355 | 0.9849935 | 1.1308156 | 1.0540394 | 0.999453 |
| Cyclin G | 1.9774908 | 2.1332314 | 1.6856765 | 1.2970321 | 1.1560766 | 1.371335 | 0.8757824 |
| Peroxisome proliferator activated receptor | 0.6596893 | 0.586164 | 1.3733292 | 1.540002 | 1.1375457 | 0.9125197 | 1.4012071 |
| gamma | | | | | | | |
| Phase-1 RCT-280 | 0.8177697 | 0.8098462 | 0.9706428 | 0.8620266 | 0.868639 | 1.4877573 | 1.3862056 |
| D-dopachrome tautomerase | 0.78515 | 0.8892555 | 0.8235343 | 0.9151852 | 0.8910152 | 1.2712693 | 1.0440753 |
| Phase-1 RCT-287 | 0.8159689 | 0.8898797 | 0.9536895 | 1.2209545 | 1.1424453 | 1.2366983 | 1.1695523 |
| Phase-1 RCT-119 | 1.0786844 | 0.8595675 | 0.6874957 | 0.6485094 | 0.7394286 | 0.6154752 | 0.8060798 |
| Thioredoxin-1 (Trx1) | 0.8518778 | 0.8609029 | 0.7468289 | 0.760199 | 0.6709374 | 0.6740977 | 0.7479546 |
| Phase-1 RCT-225 | 1.0769101 | 1.4350773 | 0.9790221 | 0.9289701 | 0.6790544 | 0.7349236 | 0.5817377 |
| Protein kinase C alpha | 1.1947953 | 1.3723167 | 0.9876699 | 1.0828557 | 1.0204557 | 1.0186974 | 0.85292 |
| Phase-1 RCT-87 | 1.0790952 | 1.0985938 | 1.0140395 | 1.0416692 | 1.0190272 | 1.0738738 | 1.094998 |
| Phase-1 RCT-59 | 1.2076896 | 1.2177812 | 0.9689841 | 1.0140747 | 1.0173962 | 1.2028736 | 0.9725691 |
| C4b-binding protein | 0.5647027 | 0.6716258 | 1.2150382 | 1.1215144 | 1.2606493 | 1.0761752 | 1.1600064 |
| Phase-1 RCT-204 | 0.9714241 | 0.7777904 | 1.0983198 | 1.1164016 | 1.0477796 | 0.8743846 | 0.7888343 |
| Carnitine palmitoyl-CoA transferase | 1.2801865 | 1.278849 | 1.203406 | 1.229704 | 1.0805371 | 1.1298245 | 1.5261058 |
| 8-oxoguanine DNA glycosytase | 0.9889084 | 1.1627195 | 1.1379384 | 1.1360916 | 1.0008862 | 0.705972 | 0.876177 |
| H-rev107 | 0.8981585 | 0.8000711 | 1.3203918 | 1.1928886 | 1.0329057 | 1.0655192 | 1.2092038 |
| Preproalbumin | 0.839368 | 0.8971706 | 2.9663517 | 3.4346387 | 4.9952097 | 8.43845 | 3.062119 |
| Phase-1 RCT-177 | 0.8287447 | 0.898407 | 1.0187092 | 1.0565392 | 1.1680287 | 1.5909325 | 1.1085057 |
| Phase-1 RCT-110 | 0.9004355 | 0.8015504 | 1.0723927 | 1.0515132 | 0.9703156 | 1.0102708 | 0.9748475 |
| Phase-1 RCT-227 | 0.8994045 | 0.854874 | 0.8182538 | 1.1084249 | 1.0847666 | 1.5027038 | 1.0517381 |
| Neuropeptide Y | 0.9670855 | 0.9274256 | 1.1288501 | 1.0788002 | 1.1272614 | 1.2508116 | 1.0645413 |
| Phase-1 RCT-277 | 0.4553956 | 0.5337867 | 1.5930319 | 0.9535381 | 1.0913203 | 1.5153064 | 1.0828774 |
| Phase-1 RCT-229 | 0.9723246 | 0.8286989 | 1.0284252 | 1.13827 | 1.1088207 | 1.2484306 | 1 |
| Phase-1 RCT-34 | 0.9541856 | 0.986385 | 0.8029276 | 1.2612526 | 1.0773942 | 1.2732087 | 1.2238476 |
| Choline kinase | 0.9855889 | 0.8386984 | 0.9720535 | 0.9211294 | 0.8452047 | 0.601532 | 0.7567457 |
| Phase-1 RCT-20 | 1.2665538 | 1.1777141 | 1.174807 | 1.1578871 | 1.0606645 | 3.1998408 | 1.7589369 |
| Phase-1 RCT-248 | 1.375568 | 1.4391384 | 0.7220593 | 0.8398526 | 0.737868 | 0.5721502 | 0.6904236 |
| | DIF | DMN | DMN | DMN | DOX | DOX | ERY |
| Compound-Dose (2) | 100 | 20 | 20 | 20 | 12 | 12 | 40 |
| Animal Number (3) | 253 | 1751 | 1752 | 1753 | 1251 | 1252 | 341 |
| Liver Toxicity Necrosis Classification (4) | no | yes | yes | yes | no | no | no |
| Gene Name (5) | | | | | | | |
| Gadd153 | 0.7835289 | 2.7634692 | 2.1360915 | 3.6335547 | 1.7136365 | 3.6575553 | 0.892915 |
| Interferon related developmental regulator | 1.2013717 | 1.2239319 | 0.9535337 | 0.9448096 | 0.7571595 | 0.7068728 | 1.1253603 |
| IFRD1 (PC4) | | | | | | | |
| ID-1 | 0.8772463 | 2.5968096 | 2.1279018 | 2.5312076 | 1.5183649 | 2.3388557 | 1.0330453 |
| Insulin-like growth factor binding protein 1 | 0.5669996 | 1.3900056 | 1.0892065 | 1.542582 | 1.3891479 | 1.6560104 | 1.715455 |
| NIPK | 0.6297803 | 3.2584634 | 1.611702 | 2.3119438 | 0.8291291 | 0.7958907 | 1.3685406 |
| Phase-1 RCT-50 | 1.1698517 | 5.8399262 | 2.5078852 | 4.758477 | 0.8698319 | 0.9943217 | 0.8401021 |
| Sarcoplasmic reticulum calcium ATPase | 0.6515051 | 0.7452555 | 0.8651663 | 0.7102866 | 0.9232515 | 1.0988955 | 1.8868299 |
| Phase-1 RCT-207 | 0.9961914 | 4.741258 | 2.6280184 | 4.4846463 | 3.9457316 | 5.4778256 | 0.9336207 |
| Acetyl-CoA carboxylase | 1.0519629 | 0.5956724 | 0.7105761 | 0.5776525 | 0.6716704 | 0.5648819 | 1.200072 |
| Cathepsin L, sequence 2 | 1.1716988 | 1.5592618 | 1.3544492 | 1.6992153 | 1.4019945 | 1.3687974 | 1.3885401 |
| Glucokinase | 0.6635521 | 0.4629579 | 0.6910814 | 0.3104679 | 0.9069638 | 1.1995432 | 0.7182848 |
| Gadd45 | 1.3279679 | 4.4704843 | 2.6021936 | 4.8645725 | 1.2906667 | 1.6169124 | 1.0843519 |
| Phase-1 RCT-18 | 0.9141742 | 1.0719212 | 0.9293661 | 1.0218002 | 0.8990918 | 1 | 0.9424956 |
| 3-beta-hydroxysteroid dehydrogenase | 1.3082374 | 0.7266947 | 0.9049369 | 0.7387941 | 1.388277 | 0.9726747 | 0.5843204 |
| (HSD3B1) | | | | | | | |
| Phase-1 RCT-221 | 0.8382678 | 1.4337856 | 1.2439082 | 1.3439407 | 1.1969742 | 1.220203 | 0.8685025 |
| Heme oxygenase | 1.8260581 | 1.6679943 | 1.0814756 | 1.1872652 | 1.0823766 | 1.1615105 | 1.1491435 |
| Cyclin dependent kinase 4 | 1.3313055 | 0.9098824 | 0.9684255 | 0.9611052 | 1.0512015 | 0.9669938 | 0.6842401 |
| Phase-1 RCT-75 | 1.0420028 | 1.5519956 | 1.1466051 | 1.5147408 | 0.9184228 | 0.9155694 | 0.8317143 |
| Phase-1 RCT-65 | 0.9831354 | 1.8276304 | 1.415835 | 1.6244664 | 0.8410533 | 0.9375822 | 0.797013 |
| Cytochrome P450 1A1 | 1.0551095 | 1.0601075 | 1.1598278 | 1.1406573 | 0.9122596 | 1.5773181 | 0.9544608 |
| Peroxisomal multifunctional enzyme type II | 1.4209152 | 0.5515961 | 0.6894243 | 0.6193892 | 1.182973 | 1.0134292 | 0.8116716 |
| DNA topoisomerase I | 0.6483043 | 0.7625399 | 0.7123134 | 0.7307804 | 1.1998571 | 1.0128113 | 1.1886564 |
| Focal adhesion kinase (pp125FAK) | 0.8600209 | 0.7493783 | 0.9017789 | 0.7627725 | 1.1540179 | 1.0581989 | 1.2310092 |
| NGF-inducible anti-proliferative putative | 0.936582 | 2.3247848 | 1.2591267 | 1.9707054 | 0.7053912 | 0.6960234 | 2.0236828 |
| secreted protein (PC3) | | | | | | | |
| Phase-1 RCT-82 | 0.8936024 | 0.9050432 | 0.8985649 | 0.8388701 | 0.8842444 | 1.122326 | 0.8663185 |
| Senescence marker protein-30 | 0.545338 | 0.2843716 | 0.4042628 | 0.30219 | 0.6757381 | 0.5034388 | 1.3258038 |
| Melanoma-associated antigen ME491 | 1.0519959 | 1.1234566 | 1.2549 | 1.7350804 | 0.9474036 | 1.0358588 | 0.8208777 |
| Activating transcription factor 3 | 1.3409036 | 4.951724 | 2.4110086 | 9.10396 | 1.0091802 | 1.1626439 | 0.6833569 |
| Tryptophan hydroxylase | 1.0091383 | 0.9625918 | 0.8797152 | 0.835977 | 0.9006427 | 0.9301914 | 1.2433642 |
| Zinc finger protein | 1.0399895 | 4.665098 | 2.8630896 | 3.9066653 | 1.0186923 | 0.9724069 | 0.8401826 |
| c-myc | 1.1384916 | 2.9950738 | 1.7897125 | 3.367003 | 0.9614662 | 1.0426886 | 0.8698621 |
| Cholesterol 7-alpha-hydroxylase P450 | 0.9437861 | 0.7143598 | 0.8142259 | 0.6898346 | 0.9173585 | 1.0213233 | 0.59038 |
| VII) | | | | | | | |
| Ferritin H-chain | 0.7172857 | 1.0038675 | 1.1405584 | 1.1268369 | 1.171653 | 1.0739478 | 0.7909544 |
| Phase-1 RCT-197 | 1.2113346 | 2.9793854 | 1.6086432 | 2.671713 | 1.1531376 | 1.4207977 | 0.8521918 |
| Phase-1 RCT-179 | 0.9508215 | 2.4748769 | 1.7155123 | 2.139141 | 1.3581831 | 1.2564949 | 1.2024245 |
| Phase-1 RCT-15 | 2.3608508 | 2.5143518 | 1.4562907 | 1.7819164 | 1.0454232 | 1.0834174 | 0.7309443 |
| Argininosuccinate lyase | 1.3584251 | 2.091064 | 1.3005798 | 1.5935695 | 1.2153777 | 1.6860768 | 1.7688313 |
| Phase-1 RCT-49 | 1.036461 | 3.159928 | 1.6832291 | 2.7350569 | 1.0461 | 1.4049759 | 1.1648395 |
| Carbonic anhydrase III | 0.4750684 | 0.3044106 | 0.5206823 | 0.1589793 | 0.6799097 | 0.4831 | 0.4522781 |
| Proliferating cell nuclear antigen gene | 0.8757937 | 1.9329227 | 1.408458 | 2.0936825 | 0.9742153 | 1.0774729 | 1.4538667 |
| Integrin beta1 | 0.8826984 | 1.3733207 | 1.1207848 | 1.3520906 | 1.1283333 | 1.3467253 | 0.868632 |
| Caspase 6 | 1.1267935 | 1.1530595 | 1.0050522 | 1.1413513 | 1.0908027 | 1.1455709 | 0.8996123 |
| Phase-1 RCT-116 | 1.4981838 | 2.804215 | 2.074911 | 2.0408783 | 0.988681 | 1.104312 | 0.879782 |
| Phase-1 RCT-109 | 0.7539058 | 1.5393132 | 1.2151245 | 1.5855896 | 1.3549582 | 1.8561252 | 1.1762372 |
| Phase-1 RCT-71 | 0.8865333 | 1.335683 | 1.178291 | 1.1005051 | 1.0353825 | 1.2753224 | 1.1003181 |
| Elongation factor-1 alpha | 1.0225891 | 1.1194668 | 0.993348 | 1.0118845 | 1.1551472 | 1.0120801 | 1.2281874 |
| Phase-1 RCT-169 | 0.8051678 | 0.9704499 | 1 | 1.299976 | 1.0704755 | 1.2276249 | 0.8280248 |
| Pyruvate kinase, muscle | 1.2632686 | 1.1352718 | 1.0516709 | 1.158779 | 0.8204168 | 0.7310895 | 0.9984887 |
| Nucleosome assembly protein | 2.1655662 | 0.9017426 | 0.818171 | 0.7942088 | 1.8015609 | 1.6744641 | 0.914977 |
| Phase-1 RCT-127 | 0.7421457 | 2.7517655 | 1.850964 | 2.4814494 | 0.7786243 | 0.852926 | 1.2374219 |
| Iron-responsive element-binding protein | 1.057322 | 0.5936948 | 0.6962427 | 0.5677659 | 0.8779098 | 0.8432872 | 1.554189 |
| Phase-1 RCT-72 | 0.9122689 | 1.4784483 | 1.0809506 | 1.689314 | 0.8992552 | 1.0525385 | 0.9672864 |
| Phase-1 RCT-144 | 1.0897225 | 3.6527135 | 2.0571349 | 3.3134623 | 1.1678252 | 1.5323609 | 0.9093057 |
| Phase-1 RCT-242 | 1.0680346 | 4.2778153 | 2.142768 | 4.0212636 | 1.4287058 | 2.512171 | 0.7075283 |
| 17-beta hydroxysteroid dehydrogenase, | 2.1285467 | 0.4785214 | 1.1547089 | 0.5146813 | 5.5649767 | 4.3197937 | 0.6748216 |
| type 2 | | | | | | | |
| Phase-1 RCT-70 | 0.8262765 | 1.1747006 | 1.0233732 | 1.2065824 | 0.839785 | 1.046701 | 1.2550893 |
| Ribosomal protein L13A | 0.6666623 | 1.5896859 | 1.2773485 | 1.5645155 | 1.6114365 | 1.9697762 | 1.2263759 |
| Cytochrome P450 2E1 | 1.4708492 | 0.4431618 | 0.3540786 | 0.5515161 | 0.818828 | 1.6510942 | 1.0911818 |
| AT-3 | 1.0109092 | 1.1667881 | 0.9615662 | 1.1649761 | 1.1071584 | 1.0340213 | 0.832381 |
| Phase-1 RCT-270 | 0.7090792 | 0.4011772 | 0.5650736 | 0.3368262 | 0.6282733 | 0.5002535 | 1.6370288 |
| Phase-1 RCT-123 | 0.9504537 | 1.010477 | 0.9495598 | 1.0569363 | 0.9031825 | 0.8483586 | 1.1020392 |
| Alpha-2-macroglobulin, sequence 2 | 0.7631831 | 0.8071213 | 0.7053223 | 0.7340056 | 0.7087691 | 0.8785034 | 1.4345533 |
| Matrix metalloproteinase-1 | 0.8213148 | 1.2833747 | 1.1878209 | 1.369808 | 1.231405 | 1.1117179 | 0.7683936 |
| Beta-tubulin, class I | 1.1705807 | 3.208725 | 1.9164867 | 3.0126839 | 0.7863578 | 0.9067696 | 0.6537312 |
| Phase-1 RCT-161 | 0.9504568 | 0.6693633 | 0.7874405 | 0.572149 | 0.5444511 | 0.6300923 | 0.7010142 |
| 14-3-3 zeta | 1.0477242 | 2.6452231 | 1.9145318 | 1.9458206 | 1.2609935 | 1.4302226 | 0.5021094 |
| Stem cell factor | 0.6011826 | 0.6286035 | 0.8574877 | 0.6282963 | 0.8738676 | 0.7211446 | 0.8375633 |
| Macrophage inflammatory protein-2 alpha | 1.0376683 | 5.8859153 | 2.080393 | 3.6116807 | 1.6221519 | 4.3107123 | 1.1607493 |
| Thymidylate synthase | 1.4905071 | 1.0370532 | 0.9428874 | 0.9632804 | 1.3209971 | 1.3720802 | 0.99652 |
| c-jun | 1.0450134 | 6.3308783 | 2.8741693 | 6.1863155 | 1.9385508 | 3.534283 | 0.9300802 |
| Superoxide dismutase Mn | 0.9883891 | 1.8340144 | 1.3580865 | 1.6279742 | 1.3337784 | 1.2124912 | 1.1260207 |
| Phase-1 RCT-73 | 1.1849988 | 0.9723752 | 0.9612783 | 0.9248407 | 0.8905695 | 0.8452399 | 1.0194948 |
| Macrophage inflammatory protein 1 alpha | 1.0913938 | 1.8791075 | 1.4821895 | 2.4320447 | 1.4520001 | 1.302664 | 0.8698251 |
| Phase-1 RCT-214 | 1.2855912 | 0.6316849 | 1.1198446 | 0.6202854 | 0.605 | 0.6077121 | 1.2238961 |
| NADH-cytochrome b5 reductase | 0.9716144 | 0.5361223 | 0.7371341 | 0.5669075 | 0.5325 | 0.4195853 | 1.2269099 |
| Interleukin-1 beta | 0.8747738 | 1.1802584 | 1.1463293 | 1.3217931 | 1.2914845 | 1.15441 | 0.8603826 |
| NADP-dependent isocitrate | 1.1332413 | 0.4825503 | 0.6111378 | 0.4465239 | 0.8449965 | 0.8249955 | 1.5759628 |
| dehydrogenase, cytosolic | | | | | | | |
| Phase-1 RCT-289 | 1.0153891 | 0.6125925 | 0.6914029 | 0.5849629 | 0.8027923 | 0.7302274 | 1.4096866 |
| Glutamine synthetase | 1.084321 | 0.5731256 | 0.690157 | 0.9628206 | 1.1978416 | 1.007456 | 0.8988083 |
| Phase-1 RCT-182 | 0.8810073 | 0.6898353 | 0.7460433 | 0.6463543 | 0.8612322 | 0.694202 | 1.4064935 |
| Ubiquitin conjugating enzyme (RAD 6 | 1.0775741 | 1.7219193 | 1.4253161 | 1.3943104 | 1.1789488 | 1.0519481 | 1.2543423 |
| homologue) | | | | | | | |
| Extracellular-signal-regulated kinase 1 | 0.4831012 | 0.6117538 | 0.7562842 | 0.4202999 | 0.6801615 | 0.4993888 | 0.5096282 |
| Emerin | 1.2263917 | 1.0394256 | 1.013423 | 0.9776732 | 0.997431 | 0.8484811 | 1.1028464 |
| Phase-1 RCT-78 | 1.0892912 | 0.9637328 | 1.0271559 | 0.917866 | 0.9806166 | 0.8944885 | 1.0629585 |
| Phase-1 RCT-212 | 0.7160929 | 1.2608567 | 1.0585625 | 0.9283719 | 0.6472754 | 0.828162 | 0.9729067 |
| Phase-1 RCT-168 | 1.1207591 | 0.52183 | 0.7787344 | 0.6053192 | 1.1333709 | 0.9855467 | 0.9996434 |
| Lysyl hydroxylase | 1.1213822 | 0.6406153 | 0.8310946 | 0.8232926 | 1.1113207 | 0.9898563 | 1.0804877 |
| Phase-1 RCT-55 | 0.7388069 | 1.0522739 | 0.9292412 | 1.0610853 | 1.0433824 | 1.0625855 | 0.6865788 |
| MHC class I antigen RT1.A1(f) alpha- | 1.1044897 | 2.5039837 | 2.375702 | 2.9335883 | 0.6816381 | 1.066051 | 0.2694188 |
| chain | | | | | | | |
| Phase-1 RCT-40 | 0.9412754 | 0.5176179 | 0.6643274 | 0.4552652 | 0.7516438 | 0.6872109 | 1.286754 |
| Cyclin G | 0.9811706 | 11.608406 | 5.425879 | 8.810581 | 2.8266072 | 4.884555 | 0.6756685 |
| Peroxisome proliferator activated receptor | 1.7575788 | 0.9871824 | 1.1446279 | 1.158231 | 1.5000836 | 1.1864322 | 0.8920239 |
| gamma | | | | | | | |
| Phase-1 RCT-280 | 1.330703 | 0.9542426 | 1.0068421 | 0.9218929 | 0.8938979 | 0.7440836 | 1.5840575 |
| D-dopachrome tautomerase | 1.033198 | 0.7300786 | 0.688565 | 0.7135617 | 0.9253599 | 0.9028862 | 1.1009005 |
| Phase-1 RCT-287 | 1.1215166 | 0.6437347 | 0.7445805 | 0.6159639 | 0.7987928 | 0.7088116 | 1.5868874 |
| Phase-1 RCT-119 | 0.8191561 | 0.4294502 | 0.4619425 | 0.3237254 | 0.6872376 | 0.6678041 | 2.1080105 |
| Thioredoxin-1 (Trx1) | 0.8464779 | 0.9804469 | 0.8567748 | 0.8671225 | 1.0541887 | 1.0880774 | 1.5495167 |
| Phase-1 RCT-225 | 0.6650739 | 1.9802423 | 1.4397321 | 1.0639042 | 0.4306505 | 0.4658952 | 0.5625975 |
| Protein kinase C alpha | 0.9110502 | 1.0322503 | 0.9865065 | 1.0526676 | 0.9643388 | 0.7678616 | 1.95717 |
| Phase-1 RCT-87 | 0.9212559 | 0.8553177 | 0.8410067 | 0.8392639 | 0.8716289 | 0.8785284 | 1.3980403 |
| Phase-1 RCT-59 | 1 | 6.071453 | 3.1821194 | 7.903795 | 3.650263 | 4.440098 | 1.1509476 |
| C4b-binding protein | 1.0131764 | 0.5747827 | 0.7250066 | 0.6409396 | 0.9642112 | 1.0091234 | 1.5504801 |
| Phase-1 RCT-204 | 0.8346456 | 1.5292451 | 1.2592149 | 1.663888 | 1.0076044 | 1.0494508 | 1.3006037 |
| Carnitine palmitoyl-CoA transferase | 1.4006947 | 2.1493251 | 1.9610192 | 2.4234111 | 1.1292717 | 1.4081326 | 0.8420576 |
| 8-oxoguanine DNA glycosylase | 0.8851944 | 1.0131531 | 1.0616709 | 1.1232765 | 1.1202703 | 0.9214398 | 0.7211685 |
| H-rev107 | 1.3396013 | 1.006677 | 0.8914694 | 0.9739823 | 0.8167735 | 0.8407175 | 1.148565 |
| Preproalbumin | 2.3950615 | 0.9891126 | 1.4207505 | 0.9394212 | 0.9566094 | 0.8977792 | 0.4137893 |
| Phase-1 RCT-177 | 1.1165385 | 0.7887878 | 0.9772732 | 0.7919344 | 1.0404531 | 0.8729369 | 1.1141635 |
| Phase-1 RCT-110 | 1.0610693 | 1.0531356 | 1.0580863 | 1.0860512 | 0.8871317 | 0.8869832 | 0.7821766 |
| Phase-1 RCT-227 | 1.0705726 | 0.795857 | 0.9731349 | 0.7670572 | 1.2551588 | 1.1012684 | 1.0825001 |
| Neuropeptide Y | 1.1624813 | 1.3750663 | 1.0834199 | 1.2606512 | 0.9625 | 0.9621885 | 1.0041858 |
| Phase-1 RCT-277 | 1.8549694 | 0.7622113 | 0.7990912 | 0.799126 | 1.1639608 | 1.0244124 | 2.4799984 |
| Phase-1 RCT-229 | 1.0343887 | 1.0684927 | 1.0318931 | 1.0566839 | 0.9453397 | 0.9619647 | 0.9547108 |
| Phase-1 RCT-34 | 1.2571117 | 0.7582375 | 0.9879892 | 0.6540166 | 1.5444446 | 2.1935241 | 0.9148544 |
| Choline kinase | 0.7542752 | 1.1687438 | 1.0452665 | 1.0650359 | 0.9354804 | 0.8979862 | 0.6273665 |
| Phase-1 RCT-20 | 1.6896739 | 1.1101228 | 1.022512 | 1.0781702 | 0.8851718 | 0.8870937 | 0.6945648 |
| Phase-1 RCT-248 | 0.730984 | 1.4070712 | 1.4083581 | 1.416326 | 0.9052239 | 1.4805955 | 0.8522354 |
| | ERY | ERY | ERY | ERY | ERY | EST | EST |
| Compound-Dose (2) | 40 | 40 | 160 | 160 | 160 | 0.1 | 0.1 |
| Animal Number (3) | 342 | 343 | 351 | 352 | 353 | 1422 | 1423 |
| Liver Toxicity Necrosis Classification (4) | no | no | no | no | no | no | no |
| Gene Name (5) | | | | | | | |
| Gadd153 | 1.0131509 | 0.9471753 | 1 | 0.9856488 | 0.8915262 | 0.7963074 | 0.9342356 |
| Interferon related developmental regulator | 1.6867974 | 1.3359295 | 1.7920336 | 1.598326 | 1.933224 | 0.8581449 | 0.8436244 |
| IFRD1 (PC4) | | | | | | | |
| ID-1 | 0.7340872 | 1.0959929 | 0.9464496 | 0.9415565 | 0.6870812 | 0.7769845 | 0.9826188 |
| Insulin-like growth factor binding protein 1 | 1.4125255 | 1.3097118 | 1.5472344 | 1.1768588 | 1.1011612 | 0.9709111 | 0.9024676 |
| NIPK | 0.7417722 | 0.7264553 | 1.0376207 | 0.8863828 | 0.7793638 | 0.7759761 | 0.9050878 |
| Phase-1 RCT-50 | 0.8314883 | 0.7083351 | 0.7090297 | 0.7922196 | 0.7402764 | 0.6842389 | 0.8163301 |
| Sarcoplasmic reticulum calcium ATPase | 1.8747405 | 1.6152213 | 1.1996129 | 1.3267893 | 0.8026229 | 0.9133492 | 0.9413347 |
| Phase-1 RCT-207 | 0.8788194 | 0.8996894 | 0.9319062 | 0.9231412 | 0.7013317 | 0.7746823 | 0.9038895 |
| Acetyl-CoA carboxylase | 1.5450044 | 1.2386752 | 1.4783483 | 1.970416 | 1.1805763 | 0.9341453 | 1.1314309 |
| Cathepsin L, sequence 2 | 1.1774081 | 1.2847914 | 1.6884935 | 1.3247303 | 1.0981913 | 1.0770248 | 0.9072449 |
| Glucokinase | 1.3223231 | 1.521782 | 0.5134712 | 0.7758472 | 0.6627622 | 1.3439231 | 1.3213698 |
| Gadd45 | 0.8390231 | 0.6449841 | 0.7526199 | 0.8417277 | 1.0102928 | 0.6496318 | 0.9500579 |
| Phase-1 RCT-18 | 1.0004622 | 0.9325709 | 0.8423117 | 0.8832883 | 0.8746072 | 0.8398449 | 0.8601617 |
| 3-beta-hydroxysteroid dehydrogenase | 1.0156319 | 0.9106228 | 0.5316004 | 0.7389792 | 0.8857649 | 1.4353075 | 1.1599782 |
| (HSD3B1) | | | | | | | |
| Phase-1 RCT-221. | 0.9583271 | 1.119747 | 1.0156262 | 0.9064395 | 1.2961017 | 1.0961291 | 1.0634004 |
| Heme oxygenase | 1.98292 | 1.3373728 | 1.1470807 | 1.402258 | 1.5469931 | 0.900802 | 1.0095888 |
| Cyclin dependent kinase 4 | 0.8201937 | 0.6605642 | 1.1296568 | 1.093581 | 0.8508177 | 0.8922606 | 0.544637 |
| Phase-1 RCT-75 | 0.6430831 | 0.5849274 | 0.8733171 | 0.7463875 | 0.8178837 | 0.978314 | 1.1075618 |
| Phase-1 RCT-65 | 0.4446246 | 0.5189442 | 0.735356 | 0.8555344 | 0.8388093 | 0.7541264 | 0.9682925 |
| Cytochrome P450 1A1 | 1.0596398 | 0.8649126 | 0.8042272 | 0.8863109 | 0.6848391 | 0.6816962 | 0.9857848 |
| Peroxisomal multifunctional enzyme type II | 0.9037236 | 0.8637435 | 1.2291635 | 1.0099907 | 1.8145205 | 1.3331455 | 1.1944213 |
| DNA topoisomerase I | 1.353949 | 1.5043219 | 1.0611229 | 1.4878061 | 2.205883 | 1.6372882 | 1.6486973 |
| Focal adhesion kinase (pp125FAK) | 1.3671725 | 1.6046762 | 1.2537608 | 1.3911356 | 1.0130914 | 0.9322475 | 1.0970393 |
| NGF-inducible anti-proliferative putative | 0.9790741 | 0.9521855 | 1.4856517 | 1.203858 | 1.5061109 | 0.8499253 | 0.9830909 |
| secreted protein (PC3) | | | | | | | |
| Phase-1 RCT-82 | 1.0540746 | 1.0047274 | 0.9024255 | 1.0506724 | 0.8876765 | 0.7518113 | 0.8997319 |
| Senescence marker protein-30 | 0.9950644 | 1.558712 | 0.9061594 | 0.8227952 | 1.1829493 | 1.5419149 | 1.2839411 |
| Melanoma-associated antigen ME491 | 1.1547494 | 1.2862153 | 1.1529762 | 1.3312546 | 1.4187051 | 1.1636863 | 1.054955 |
| Activating transcription factor 3 | 0.8624018 | 0.8110191 | 0.6593128 | 0.743288 | 0.6863983 | 1.1154406 | 1.2974913 |
| Tryptophan hydroxylase | 0.7461352 | 0.7840836 | 1.0419278 | 1.0734322 | 1.0431467 | 1.123614 | 1.0263213 |
| Zinc finger protein | 1.3894747 | 0.9281926 | 0.9492554 | 0.8141191 | 0.7466667 | 1.1954583 | 1.2009614 |
| c-myc | 1.3106813 | 0.8585084 | 0.625935 | 0.9444807 | 0.7075344 | 0.7123765 | 0.9769859 |
| Cholesterol 7-alpha-hydroxylase P450 | 1.7674837 | 1.2059116 | 0.6983711 | 0.8654006 | 0.5698858 | 1.1789088 | 1.1459078 |
| VII) | | | | | | | |
| Ferritin H-chain | 0.5798258 | 0.7978475 | 0.914418 | 0.7953628 | 1.3405317 | 1.261707 | 0.998283 |
| Phase-1 RCT-197 | 1.3038616 | 1.4111732 | 1.2599512 | 1.0100839 | 1.0086739 | 0.7293165 | 0.8733414 |
| Phase-1 RCT-179 | 1.4668504 | 1.7654766 | 1.71527 | 1.0910583 | 0.7573277 | 0.9701836 | 0.8730488 |
| Phase-1 RCT-15 | 1.2090274 | 0.7027423 | 0.9125265 | 1.0356091 | 1.0584905 | 0.9674718 | 0.9195526 |
| Argininosuccinate lyase | 0.5524889 | 0.4614466 | 1.193287 | 1.3112205 | 1.9899453 | 1.0433547 | 0.9880086 |
| Phase-1 RCT-49 | 1.1186069 | 1.2124461 | 1.2197388 | 1.5133139 | 0.992198 | 0.8082328 | 0.8758459 |
| Carbonic anhydrase III | 0.3881274 | 0.3381833 | 0.1545165 | 0.2702588 | 0.5410996 | 1.8491436 | 0.894806 |
| Proliferating cell nuclear antigen gene | 1.1128132 | 1.0369985 | 1.1363467 | 1.3857287 | 0.9707131 | 0.7581933 | 0.8950108 |
| Integrin beta1 | 0.91538 | 0.9280598 | 0.9461837 | 0.8459505 | 0.6858171 | 0.981594 | 0.9450706 |
| Caspase 6 | 0.7886009 | 0.8406389 | 1.0676599 | 0.958185 | 0.7816393 | 0.9848979 | 0.9745107 |
| Phase-1 RCT-116 | 0.3531345 | 0.2786272 | 0.6918442 | 0.9034101 | 0.8918682 | 0.4914708 | 0.66062 |
| Phase-1 RCT-109 | 0.874524 | 0.8593291 | 1.3883901 | 1.3994707 | 1.318573 | 1.2119268 | 1.1664286 |
| Phase-1 RCT-71 | 1.021799 | 0.8903363 | 1.0096934 | 1.17089 | 1.1079706 | 0.7751497 | 0.9176413 |
| Elongation factor-1 alpha | 0.9384159 | 1.1250173 | 1.3803848 | 1.5621709 | 2.4764445 | 1.4100875 | 1.1791188 |
| Phase-1 RCT-169 | 1.3279272 | 1.0032408 | 0.656775 | 1.1181076 | 0.678765 | 0.6488193 | 0.8776336 |
| Pyruvate kinase, muscle | 0.8123157 | 0.6220736 | 0.8369383 | 0.9284468 | 0.6936923 | 0.9047543 | 0.936325 |
| Nucleosome assembly protein | 1.2669642 | 1.1990772 | 1.106503 | 1.0415143 | 1.3950613 | 0.7923462 | 0.7260546 |
| Phase-1 RCT-127 | 1.4223213 | 1.5873238 | 0.876962 | 1.4400619 | 1.0136966 | 0.8540568 | 0.9440831 |
| Iron-responsive element-binding protein | 0.8235227 | 1.1671485 | 1.4635953 | 1.1866817 | 1.1498272 | 1.1894493 | 1.0927458 |
| Phase-1 RCT-72 | 1.150724 | 0.9855813 | 0.8329193 | 0.9864884 | 0.7644585 | 0.6987487 | 0.936699 |
| Phase-1 RCT-144 | 1.5099847 | 1.8019848 | 1.3303572 | 1.9731643 | 1.1198386 | 0.7681992 | 0.9255725 |
| Phase-1 RCT-242 | 1.0646973 | 0.98813 | 0.86059 | 0.9826224 | 0.879656 | 0.6788809 | 0.8376394 |
| 17-beta hydroxysteroid dehydrogenase, | 1.171096 | 0.955455 | 0.9186965 | 0.8705337 | 1.2867705 | 0.7192826 | 0.6400901 |
| type 2 | | | | | | | |
| Phase-1 RCT-70 | 1.1414571 | 0.9677786 | 1.2138782 | 1.1109265 | 1.0010089 | 1.0179657 | 1.0016474 |
| Ribosomal protein L13A | 0.8927635 | 0.6810235 | 1.612297 | 1.3930826 | 1.2527714 | 1.2477404 | 1.3320664 |
| Cytochrome P450 2E1 | 0.6713246 | 0.8082452 | 0.7140248 | 1.0402614 | 0.9379149 | 1.2084614 | 0.9576892 |
| AT-3 | 1.5656103 | 1.1600322 | 0.712472 | 0.9217928 | 0.9106228 | 0.8068411 | 1.0236082 |
| Phase-1 RCT-270 | 0.7148238 | 0.7708334 | 0.9427805 | 1.0993949 | 1.1445255 | 1.4826891 | 1.0451792 |
| Phase-1 RCT-123 | 0.9956724 | 1.0089142 | 1.0251856 | 0.962006 | 1.0249429 | 0.7607854 | 0.9022651 |
| Alpha-2-macroglobulin, sequence 2 | 0.8264394 | 0.8989087 | 1.3866827 | 1.6776382 | 1.0396745 | 1.1021883 | 0.9537591 |
| Matrix metalloproteinase-1 | 0.6648716 | 0.8457176 | 0.9538271 | 0.8153479 | 1.0341309 | 1.3212502 | 0.9954585 |
| Beta-tubulin, class I | 0.2807447 | 0.4109575 | 0.5866685 | 0.9401471 | 1.0975928 | 0.984358 | 1.0475734 |
| Phase-1 RCT-161 | 0.3249824 | 0.2946154 | 0.8170066 | 0.8921009 | 0.7487954 | 0.7964858 | 0.7262945 |
| 14-3-3 zeta | 0.6422179 | 0.601244 | 0.5690116 | 0.7600928 | 0.6912248 | 0.6859844 | 0.8145136 |
| Stem cell factor | 0.9502631 | 1.0364805 | 0.7821227 | 0.7991197 | 0.7163438 | 1.2561315 | 1.1956146 |
| Macrophage inflammatory protein-2 alpha | 1.5312365 | 0.7668536 | 1.0086368 | 1.0556844 | 0.9880999 | 0.812092 | 0.9934964 |
| Thymidylate synthase | 1.0838666 | 1.0750396 | 0.7240039 | 0.865212 | 0.5495735 | 0.7858306 | 0.9957789 |
| c-jun | 0.763423 | 0.8786777 | 1.0692589 | 1.0173876 | 0.9150853 | 0.8925537 | 1.0495192 |
| Superoxide dismutase Mn | 1.0520145 | 1.0608006 | 1.3213862 | 1.4110487 | 1.4506973 | 1.1454229 | 1.0831802 |
| Phase-1 RCT-73 | 0.836229 | 0.8481513 | 1.1042784 | 1.0219548 | 1.3858722 | 0.9591825 | 0.8852518 |
| Macrophage inflammatory protein 1 alpha | 1.0516834 | 0.9735724 | 0.8189716 | 0.9364313 | 0.8681046 | 0.6618428 | 0.8469229 |
| Phase-1 RCT-214 | 0.8048398 | 0.9323912 | 0.8411842 | 0.8901988 | 0.8446674 | 0.9711207 | 1.2873286 |
| NADH-cytochrome b5 reductase | 0.6857025 | 0.7786142 | 0.901718 | 0.9125163 | 0.9692691 | 1.4715375 | 1.3073021 |
| Interleukin-1 beta | 0.7720144 | 0.9198625 | 0.8532499 | 1.0709758 | 0.796042 | 1.3018434 | 1.0317203 |
| NADP-dependent isocitrate | 0.7016332 | 1.1244615 | 1.2918222 | 1.0436294 | 1.1470474 | 1.1978151 | 1.1521425 |
| dehydrogenase, cytosolic | | | | | | | |
| Phase-1 RCT-289 | 0.8338496 | 1.0004985 | 1.1396871 | 1.143574 | 1.150601 | 1.3554294 | 1.1212798 |
| Glutamine synthetase | 1.5645282 | 1.6637605 | 1.4361356 | 1.034441 | 1.5683843 | 1.5630064 | 1.1189969 |
| Phase-1 RCT-182 | 0.981078 | 1.3015753 | 0.9955105 | 1.3586593 | 1.2771626 | 1.6049628 | 1.1905062 |
| Ubiquitin conjugating enzyme (RAD 6 | 1.3448393 | 1.3884952 | 1.5298707 | 1.369906 | 1.1755967 | 1 | 1.1235083 |
| homologue) | | | | | | | |
| Extracellular-signal-regulated kinase 1 | 0.6574135 | 0.5709472 | 0.2205146 | 0.3789895 | 0.5109311 | 1.3551108 | 0.916277 |
| Emerin | 0.7420299 | 0.8208376 | 1.1064181 | 0.7562854 | 0.7439269 | 0.9616956 | 1.0572027 |
| Phase-1 RCT-78 | 0.9187272 | 0.862436 | 0.7939779 | 0.8995986 | 1.0274034 | 1.0830078 | 0.999565 |
| Phase-1 RCT-212 | 1.0761435 | 0.9147837 | 0.931778 | 1.130138 | 0.9721732 | 0.7315075 | 0.8424636 |
| Phase-1 RCT-168 | 1.0705185 | 1.3650427 | 1.1447351 | 0.94151 | 1.172966 | 1.3376229 | 1.0826093 |
| Lysyl hydroxylase | 1.2514198 | 1.4230347 | 1.5086398 | 1.2550412 | 1.6074142 | 1.5533127 | 1.2078619 |
| Phase-1 RCT-55 | 1.0370266 | 1.007302 | 0.9881374 | 1.0385654 | 0.9657497 | 0.882597 | 0.9515997 |
| MHC class I antigen RT1.A1(f) alpha- | 0.4321977 | 0.384503 | 0.3659965 | 0.4277926 | 0.5429683 | 0.7571532 | 0.9681933 |
| chain | | | | | | | |
| Phase-1 RCT-40 | 1.0423954 | 1.291455 | 1.3408848 | 1.1141518 | 1.5901107 | 1.1488858 | 0.9998204 |
| Cyclin G | 1.2878695 | 1.2771572 | 1.2481596 | 0.839044 | 0.8426447 | 0.7168882 | 0.9429215 |
| Peroxisome proliferator activated receptor | 0.7371407 | 0.7330893 | 0.7332332 | 0.85286 | 1.0430323 | 1.0643578 | 1.2538161 |
| gamma | | | | | | | |
| Phase-1 RCT-280 | 1.0357163 | 1.2246869 | 1.476581 | 1.0611261 | 1.8882008 | 1.3969136 | 1.2756737 |
| D-dopachrome tautomerase | 0.6894233 | 0.9131733 | 0.9162919 | 1.1795444 | 1.7122018 | 1.2438076 | 1.0254271 |
| Phase-1 RCT-287 | 0.9610177 | 1.2148949 | 1.7886199 | 1.479234 | 1.6762542 | 0.8984547 | 0.9380334 |
| Phase-1 RCT-119 | 0.9841449 | 0.8091317 | 1.2393218 | 1.4355767 | 1.3798809 | 1.563357 | 1.2553256 |
| Thioredoxin-1 (Trx1) | 1.1148903 | 1.2236829 | 1.6762501 | 1.6292542 | 1.8406374 | 0.9015483 | 0.8520425 |
| Phase-1 RCT-225 | 0.9886569 | 0.6541455 | 0.5924696 | 0.7503481 | 0.8195771 | 0.9421056 | 0.8247786 |
| Protein kinase C alpha | 1.322306 | 1.3798138 | 1.4952191 | 1.3766813 | 1.2209836 | 1.4326771 | 1.2412567 |
| Phase-1 RCT-87 | 1.0903051 | 1.1809626 | 1.2704817 | 1.4782107 | 1.1348828 | 0.8629355 | 0.9160267 |
| Phase-1 RCT-59 | 1.1509278 | 1.1361003 | 1.2230527 | 0.9926949 | 1.1881704 | 1.0313685 | 0.9696884 |
| C4b-binding protein | 1.2537663 | 1.3449256 | 1.3280367 | 1.4374032 | 1.7904607 | 1.0771612 | 1.1564987 |
| Phase-1 RCT-204 | 0.9281276 | 1.001132 | 1.1640625 | 1.0434731 | 1.098005 | 0.8486773 | 0.8733006 |
| Carnitine palmitoyl-CoA transferase | 0.6469292 | 0.9145371 | 0.9286037 | 0.8107116 | 0.8052735 | 0.7683026 | 0.8239064 |
| 8-oxoguanine DNA glycosylase | 1.1213989 | 1.3804375 | 0.9825917 | 0.9889684 | 0.8375036 | 0.8907017 | 0.9957311 |
| H-rev107 | 0.6165416 | 1.0352539 | 0.7763128 | 1.1132673 | 0.8779496 | 0.8074973 | 0.8650863 |
| Preproalbumin | 0.3790047 | 0.2406324 | 0.5308922 | 0.6073061 | 0.9899747 | 1.0581433 | 0.8923597 |
| Phase-1 RCT-177 | 0.7716496 | 0.9684581 | 1.2376125 | 1.0735385 | 1.6153178 | 0.9743791 | 0.8646798 |
| Phase-1 RCT-110 | 0.9535732 | 1.0001798 | 0.4645274 | 0.8380237 | 0.7811332 | 0.7752954 | 0.8421447 |
| Phase-1 RCT-227 | 0.9475123 | 0.8242272 | 0.810636 | 0.8839617 | 1.1547942 | 1.0716959 | 0.8830223 |
| Neuropeptide Y | 1.1775752 | 0.9499241 | 0.8545064 | 1.1144443 | 1.0307418 | 0.9791156 | 1.0761689 |
| Phase-1 RCT-277 | 1.1709756 | 1.6129565 | 0.7079576 | 1.6426915 | 1.6770371 | 1.1141748 | 1.3397336 |
| Phase-1 RCT-229 | 1.3057052 | 0.8955523 | 0.8156007 | 0.9195252 | 0.8416923 | 0.8592279 | 0.9958171 |
| Phase-1 RCT-34 | 0.7270933 | 0.8013015 | 0.7514502 | 0.855899 | 0.7197377 | 1.1397501 | 0.9509527 |
| Choline kinase | 0.7441332 | 0.7988909 | 0.6021789 | 0.7848785 | 0.6368336 | 0.6087287 | 0.9470288 |
| Phase-1 RCT-20 | 1.161555 | 0.9287823 | 0.7658473 | 0.8302552 | 0.6394958 | 0.6899375 | 0.8632728 |
| Phase-1 RCT-248 | 0.9325866 | 0.9898763 | 0.863609 | 1.0583073 | 1.0489224 | 1.0157247 | 1.0513474 |
| | EST | EST | EST | ETH | ETH | ETH | GAN |
| Compound-Dose (2) | 0.4 | 0.4 | 0.4 | 2500 | 2500 | 2500 | 50 |
| Animal Number (3) | 1431 | 1432 | 1433 | 131 | 132 | 133 | 2441 |
| Liver Toxicity Necrosis Classification (4) | no | no | no | no | no | no | no |
| Gene Name (5) | | | | | | | |
| Gadd153 | 0.7583081 | 0.7698525 | 0.9120985 | 1.1874509 | 1.0804772 | 1.2623208 | 0.8603386 |
| Interferon related developmental regulator | 1.001286 | 1.0996817 | 1.1304364 | 1.006948 | 1.095503 | 1.2279831 | 1.3860478 |
| IFRD1 (PC4) | | | | | | | |
| ID-1 | 0.884069 | 0.8581831 | 0.8763377 | 0.9775443 | 1.0918127 | 1.0781168 | 0.933121 |
| Insulin-like growth factor binding protein 1 | 1.0365711 | 0.8608601 | 1.5081958 | 0.878446 | 0.8526927 | 0.8655914 | 0.9286351 |
| NIPK | 0.9946584 | 0.7576783 | 1.006434 | 0.9208556 | 1.0308541 | 0.9778167 | 0.9909315 |
| Phase-1 RCT-50 | 0.7670839 | 0.7466373 | 0.6471496 | 1.2012886 | 0.9791218 | 1.014139 | 1.0153555 |
| Sarcoplasmic reticulum calcium ATPase | 0.7865374 | 0.9722935 | 1.1977534 | 1.3622067 | 1.0700077 | 1.1980573 | 0.8412523 |
| Phase-1 RCT-207 | 0.8604938 | 0.7317975 | 0.6295563 | 1.1092855 | 1.0044167 | 1.0210335 | 0.9323568 |
| Acetyl-CoA carboxylase | 0.9466537 | 1.2056348 | 1.0184463 | 0.9486445 | 1.1001056 | 0.9098421 | 0.8493068 |
| Cathepsin L, sequence 2 | 1.0091324 | 1.4368359 | 1.1369851 | 0.9241644 | 1.1408347 | 1.1029679 | 1.1618222 |
| Glucokinase | 1.1481795 | 1.371239 | 1.8385235 | 1.0129913 | 0.9670098 | 1.0549194 | 0.6670471 |
| Gadd45 | 0.734883 | 0.7567283 | 0.7942495 | 1.8339617 | 1.0281488 | 1.5438823 | 0.9799284 |
| Phase-1 RCT-18 | 0.8924657 | 0.9490553 | 0.8767011 | 0.8833316 | 1.0495888 | 0.915588 | 0.8922961 |
| 3-beta-hydroxysteroid dehydrogenase | 1.2762828 | 1.3139546 | 0.9772087 | 0.7692739 | 0.9491699 | 0.6263946 | 1.3121604 |
| (HSD3B1) | | | | | | | |
| Phase-1 RCT-221 | 1.0356052 | 1.0647916 | 1.2703807 | 0.8347601 | 1.2746607 | 0.8924251 | 0.7823151 |
| Heme oxygenase | 0.7130534 | 1.1116625 | 0.9067209 | 1.2784023 | 0.9541441 | 0.9379441 | 1.2820069 |
| Cyclin dependent kinase 4 | 0.9255791 | 0.9216291 | 0.8444917 | 1.0397849 | 0.958056 | 1.1758977 | 1.0276918 |
| Phase-1 RCT-75 | 1.020117 | 0.8959627 | 0.8070684 | 1.097275 | 1.2415928 | 1.0507032 | 1.0551605 |
| Phase-1 RCT-65 | 0.7586916 | 0.6571576 | 0.7016456 | 1.1419393 | 1.1070446 | 0.8952279 | 0.9099855 |
| Cytochrome P450 1A1 | 0.8273394 | 1.1778455 | 0.7946378 | 1.285492 | 0.8539681 | 1.288459 | 1.2074642 |
| Peroxisomal multifunctional enzyme type II | 1.2086265 | 1.1981788 | 1.01007 | 0.9280307 | 1.118123 | 0.9031642 | 1.3529346 |
| DNA topoisomerase I | 1.8595204 | 1.4872819 | 1.8480041 | 0.7865569 | 1.1197311 | 0.9137864 | 1.1671149 |
| Focal adhesion kinase (pp125FAK) | 1.0617284 | 1.1731706 | 1.0618595 | 0.8702077 | 0.8051527 | 1.0293117 | 1.2581793 |
| NGF-inducible anti-proliferative putative | 0.9753243 | 0.9171475 | 1.3298756 | 0.8220695 | 0.8147953 | 0.8885866 | 1.1781976 |
| secreted protein (PC3) | | | | | | | |
| Phase-1 RCT-82 | 0.8659771 | 1.4905599 | 0.8854067 | 1.1209044 | 1.034864 | 1.0342131 | 0.9278823 |
| Senescence marker protein-30 | 1.7852149 | 1.2896401 | 2.0485046 | 0.5668374 | 0.7300475 | 0.7186121 | 1.0314611 |
| Melanoma-associated antigen ME491 | 1.1410657 | 1.138584 | 1.0716753 | 1.0264399 | 0.9282906 | 1.0704973 | 1.0858425 |
| Activating transcription factor 3 | 1.1226995 | 0.9317565 | 0.7945917 | 1.1823715 | 1.1963793 | 1.1140978 | 0.9138985 |
| Tryptophan hydroxylase | 0.8210166 | 0.8562038 | 0.8573077 | 1.0787637 | 0.9945273 | 1.0336542 | 1.0870714 |
| Zinc finger protein | 1.0538801 | 1.0335635 | 0.7848162 | 0.9077632 | 1.0340881 | 0.8755602 | 1.1337997 |
| c-myc | 0.8141762 | 0.8086494 | 0.6411319 | 1.3958893 | 0.8888675 | 1.3064717 | 1.0679102 |
| Cholesterol 7-alpha-hydroxylase P450 | 1.3262137 | 1.4430654 | 1.253966 | 1.0400044 | 0.7799749 | 1.1664655 | 0.9816918 |
| VII) | | | | | | | |
| Ferritin H-chain | 1.2793036 | 1.1860831 | 1.4408659 | 0.6749139 | 0.913588 | 0.7940972 | 1.0266696 |
| Phase-1 RCT-197 | 0.6326824 | 0.8402675 | 0.89183 | 1.0415212 | 1.0492218 | 1.2352865 | 1.0782217 |
| Phase-1 RCT-179 | 1.0050968 | 1.0468878 | 1.107739 | 0.7940169 | 0.960488 | 0.8571287 | 1.0903771 |
| Phase-1 RCT-15 | 0.7134185 | 0.6525286 | 0.6696647 | 1.1304988 | 1.1490401 | 0.6889578 | 1.1593689 |
| Argininosuccinate lyase | 1.1607779 | 1.0989169 | 0.8088696 | 0.9873684 | 0.9788706 | 0.908393 | 1.4169972 |
| Phase-1 RCT-49 | 0.8850216 | 1.0885545 | 1.0527546 | 0.9318548 | 1.0897816 | 1.09795 | 0.9540139 |
| Carbonic anhydrase III | 1.2231811 | 0.5585451 | 1.0962778 | 0.4925981 | 0.3665705 | 0.272682 | 0.5880975 |
| Proliferating cell nuclear antigen gene | 0.8150545 | 0.9085831 | 0.9223794 | 1.2937853 | 0.8267971 | 1.2517221 | 0.8121697 |
| Integrin beta1 | 0.9525597 | 0.8549941 | 0.9678363 | 1.300398 | 0.94458 | 1.2997758 | 1.0244067 |
| Caspase 6 | 0.8927584 | 0.8832731 | 0.9826469 | 1.2284529 | 1.0596352 | 1.380923 | 1.0215634 |
| Phase-1 RCT-116 | 0.5438997 | 0.4776347 | 0.4814604 | 1.1655014 | 1.1333174 | 0.8168195 | 1.1970849 |
| Phase-1 RCT-109 | 1.435941 | 1.3927195 | 1.6048622 | 0.8536423 | 0.9940829 | 0.8617926 | 1.0467349 |
| Phase-1 RCT-71 | 0.8122494 | 1.0540559 | 0.8327419 | 0.9916235 | 0.9306421 | 0.840392 | 1.0546343 |
| Elongation factor-1 alpha | 1.3686469 | 1.2923751 | 1.1689371 | 0.6774882 | 1.1998724 | 0.816362 | 1.0792495 |
| Phase-1 RCT-169 | 0.5146973 | 0.7501223 | 0.9086439 | 1.2657778 | 1.0787144 | 1.0394231 | 1.0613164 |
| Pyruvate kinase, muscle | 0.9326756 | 1.0181215 | 0.8582439 | 0.9331533 | 1.2481794 | 1.10982 | 0.9593846 |
| Nucleosome assembly protein | 1.078611 1 | 0.8911851 | 0.9528492 | 0.6964823 | 0.7626748 | 0.8478584 | 0.8093572 |
| Phase-1 RCT-127 | 1.046854 | 0.9635861 | 1.08892 | 0.91751 | 0.8532536 | 0.9509718 | 1.126501 |
| Iron-responsive element-binding protein | 1.1374741 | 1.0109282 | 1.1224977 | 1.3855858 | 1.201415 | 1.4941527 | 0.9774711 |
| Phase-1 RCT-72 | 0.5026922 | 0.5953944 | 0.7881503 | 1.3795601 | 1.0387912 | 1.1626867 | 0.908513 |
| Phase-1 RCT-144 | 1.0001702 | 1.1296295 | 0.9777842 | 1.0644376 | 1.0153602 | 1.0941297 | 0.9349667 |
| Phase-1 RCT-242 | 0.8252167 | 1.3312824 | 0.7098084 | 1.3371048 | 0.9304188 | 1.1935967 | 0.9684542 |
| 17-beta hydroxysteroid dehydrogenase, | 1.0050323 | 0.665345 | 0.8988867 | 0.8477258 | 0.759887 | 0.8415563 | 0.7975014 |
| type 2 | | | | | | | |
| Phase-1 RCT-70 | 0.9158249 | 1.0304717 | 0.8884482 | 1.1311026 | 1.2465945 | 0.9209083 | 0.8596746 |
| Ribosomal protein L13A | 1.4874643 | 1.2703654 | 1.6974485 | 0.725181 | 1.0808575 | 0.7949693 | 0.946975 |
| Cytochrome P450 2E1 | 0.9481482 | 0.9704424 | 1.0339531 | 0.9555704 | 0.800234 | 1.1395278 | 1.0104347 |
| AT-3 | 0.8949907 | 0.9058294 | 0.8574736 | 0.8748853 | 0.9809812 | 1.1611369 | 1.1031704 |
| Phase-1 RCT-270 | 1.2079656 | 1.2068535 | 1.3416092 | 0.9044092 | 0.9920289 | 1.0571306 | 0.8544738 |
| Phase-1 RCT-123 | 0.9110397 | 0.9114224 | 0.8430084 | 1.0634174 | 0.9727401 | 1.0716985 | 1.0048393 |
| Alpha-2-macroglobulin, sequence 2 | 0.870599 | 1.1371822 | 0.9455547 | 0.7921562 | 0.7834363 | 0.6754215 | 1.3074244 |
| Matrix metalloproteinase-1 | 1.2021214 | 1.1424415 | 1.3794646 | 0.7566267 | 0.9719384 | 0.8621512 | 1.0250927 |
| Beta-tubulin, class I | 1.1117284 | 0.9267524 | 0.8522871 | 0.8049175 | 0.9024373 | 0.7973308 | 0.795205 |
| Phase-1 RCT-161 | 0.7817141 | 2.4560826 | 0.7034597 | 1.0535109 | 0.7583721 | 0.7825052 | 0.7647272 |
| 14-3-3 zeta | 0.7807224 | 0.6778676 | 0.5988513 | 1.0457047 | 1.014135 | 1.0005802 | 1.0594461 |
| Stem cell factor | 0.861441 | 0.8286937 | 1.0669761 | 0.8631797 | 0.8771512 | 0.839173 | 1.0048153 |
| Macrophage inflammatory protein-2 alpha | 0.6536919 | 0.893988 | 0.6906793 | 1.8744742 | 1.1779593 | 1.6661495 | 1.1650777 |
| Thymidylate synthase | 0.8088392 | 0.7231497 | 0.6710587 | 1.3389996 | 0.8039407 | 1.3895127 | 0.9861366 |
| c-jun | 0.6597843 | 0.7935766 | 0.6889894 | 1.594034 | 0.9953411 | 1.2989212 | 0.9201561 |
| Superoxide dismutase Mn | 1.088066 | 1.1309911 | 0.9818796 | 1.1272775 | 0.921368 | 1.1746732 | 0.9673803 |
| Phase-1 RCT-73 | 1.0457087 | 1.0116353 | 1.0344901 | 0.8820209 | 0.9588637 | 0.8239133 | 1.2085308 |
| Macrophage inflammatory protein-1 alpha | 0.4831599 | 0.6374102 | 0.4924438 | 1.232168 | 0.9600214 | 1.2350811 | 1.0909677 |
| Phase-1 RCT-214 | 1.156774 | 0.9208229 | 1.0589978 | 0.8612923 | 1.0531908 | 0.8455996 | 1.0080605 |
| NADH-cytochrome b5 reductase | 1.3602033 | 1.0804011 | 1.4745059 | 1.0822381 | 1.1078827 | 1.015317 | 0.876952 |
| Interleukin-1 beta | 1.1057063 | 1.1057261 | 1.2346438 | 0.9397197 | 1.0233185 | 1.0588788 | 1.0088378 |
| NADP-dependent isocitrate | 1.2737777 | 1.1491959 | 1.3239802 | 0.787567 | 0.8268277 | 0.8448204 | 1.0262548 |
| dehydrogenase, cytosolic | | | | | | | |
| Phase-1 RCT-289 | 1.2001828 | 1.1137056 | 1.3812559 | 0.6661381 | 0.7671452 | 0.7892287 | 0.9443383 |
| Glutamine synthetase | 1.3755157 | 1.1957835 | 1.3032209 | 0.8167889 | 1.0235181 | 1.0345902 | 1.1066206 |
| Phase-1 RCT-182 | 1.3387742 | 1.1643863 | 1.216596 | 0.7467043 | 1.1178029 | 0.8601782 | 0.9104491 |
| Ubiquitin conjugating enzyme (RAD 6 | 1.1700889 | 1.3017168 | 1.4262296 | 0.7525781 | 0.9689129 | 0.9847508 | 1.244106 |
| homologue) | | | | | | | |
| Extracellular-signal-regulated kinase 1 | 0.8741012 | 0.5409387 | 0.7917188 | 0.8020002 | 0.7161292 | 0.5582217 | 0.9275815 |
| Emerin | 1.1077341 | 0.8291246 | 0.8847648 | 0.8820133 | 1.0722513 | 0.9613263 | 0.9293455 |
| Phase-1 RCT-78 | 0.9842194 | 0.9563347 | 0.9348434 | 0.8889429 | 1.0126139 | 0.9263166 | 0.9971605 |
| Phase-1 RCT-212 | 0.5962918 | 0.854425 | 1.0236936 | 0.8793683 | 0.7236772 | 0.8517733 | 1.0374604 |
| Phase-1 RCT-168 | 1.2553726 | 1.2274389 | 1.4154276 | 0.7620825 | 1.2491962 | 1.0798645 | 0.8647597 |
| Lysyl hydroxylase | 1.2146837 | 1.3479693 | 1.3047473 | 0.8151518 | 1.0319768 | 1.0298262 | 1.0653174 |
| Phase-1 RCT-55 | 0.879663 | 0.6863385 | 1.0278572 | 0.9126117 | 0.8802429 | 0.7812859 | 0.7255867 |
| MHC class I antigen RT1.A1(f) alpha- | 0.6395994 | 0.7121908 | 0.6002991 | 1.6405619 | 1.6095062 | 0.882232 | 0.9585427 |
| chain | | | | | | | |
| Phase-1 RCT-40 | 1.0830338 | 1.0027827 | 1.041308 | 0.8026694 | 0.9889929 | 0.8268415 | 1.0836779 |
| Cyclin G | 0.7155522 | 0.6950899 | 0.5960293 | 1.4022173 | 1.0101626 | 1.2929996 | 0.9957031 |
| Peroxisome proliferator activated receptor | 0.8843854 | 0.7232987 | 0.736696 | 1.2497065 | 0.8580204 | 1.2166704 | 0.9143531 |
| gamma | | | | | | | |
| Phase-1 RCT-280 | 1.3155366 | 1.2898817 | 1.0758455 | 0.8511196 | 1.0523498 | 0.8232631 | 1.4219652 |
| D-dopachrome tautomerase | 1.0051653 | 0.9620568 | 1.2246931 | 0.6749989 | 0.7874721 | 0.582478 | 1.0955334 |
| Phase-1 RCT-287 | 1.0070418 | 0.9916791 | 0.9809035 | 0.9129724 | 0.9930888 | 0.9282041 | 1.1749226 |
| Phase-1 RCT-119 | 1.2167274 | 1.3616536 | 1.2856426 | 0.9509191 | 1.1892052 | 0.8654614 | 1.0582108 |
| Thioredoxin-1 (Trx1) | 0.8662677 | 0.9274285 | 1.2117405 | 0.8555372 | 0.8410054 | 0.9198303 | 1.1532905 |
| Phase-1 RCT-225 | 0.7344037 | 0.9363172 | 1.0031679 | 0.7578877 | 0.6707206 | 0.7501526 | 0.9744781 |
| Protein kinase C alpha | 1.2192311 | 1.3185242 | 1.3396301 | 1.0326285 | 0.9227675 | 1.1990436 | 0.9184683 |
| Phase-1 RCT-87 | 0.9240755 | 1.9859939 | 0.9971004 | 1.056428 | 0.8913999 | 1.0139613 | 0.9717036 |
| Phase-1 RCT-59 | 1.1328888 | 1.1499255 | 0.8764969 | 0.9291191 | 1.1524923 | 0.9575315 | 1.0118767 |
| C4b-binding protein | 1.202521 | 1.1237297 | 1.1046677 | 0.7481521 | 0.9953318 | 0.8215811 | 1.3808086 |
| Phase-1 RCT-204 | 1.0709085 | 0.9666277 | 0.9695225 | 1.0442817 | 0.9741399 | 0.972427 | 1.0932584 |
| Carnitine palmitoyl-CoA transferase | 0.7734776 | 0.6836717 | 0.5770065 | 1.2980025 | 0.9861904 | 1.1925623 | 0.8941209 |
| 8-oxoguanine DNA glycosylase | 0.8031693 | 0.8431655 | 0.8255994 | 1.1797314 | 1.0118593 | 1.1509423 | 0.8836221 |
| H-rev107 | 0.8491399 | 0.833738 | 0.7324302 | 1.1104679 | 0.9699454 | 1.1077619 | 1.0424954 |
| Preproalbumin | 0.9256532 | 0.658998 | 0.5969732 | 1.0323056 | 1.1199471 | 0.605579 | 1.4249024 |
| Phase-1 RCT-177 | 1.1035552 | 1 | 1.1046382 | 0.8708686 | 1.0178326 | 0.8067791 | 1.3251534 |
| Phase-1 RCT-110 | 0.8425152 | 0.8108224 | 0.768342 | 1.0966513 | 0.9109964 | 1.0533818 | 0.8745589 |
| Phase-1 RCT-227 | 1.0170791 | 1.0507699 | 0.9782375 | 0.8431815 | 1.0271181 | 0.9168049 | 1.0997632 |
| Neuropeptide Y | 0.9969136 | 0.9063368 | 0.9143534 | 0.933259 | 1.0613875 | 0.9916257 | 0.9467704 |
| Phase-1 RCT-277 | 1.4484167 | 1.9124676 | 1.5849967 | 0.8180931 | 1.2988164 | 1.0839533 | 1.0817962 |
| Phase-1 RCT-229 | 0.7888463 | 0.8319213 | 0.8140638 | 0.9917877 | 1.00168 | 0.9455593 | 0.8976886 |
| Phase-1 RCT-34 | 0.8389707 | 0.6816721 | 0.9782218 | 1.1307684 | 1.2562938 | 0.9021536 | 0.8324536 |
| Choline kinase | 0.6360675 | 0.6894325 | 0.7461103 | 1.4133804 | 1.1113985 | 1.2417948 | 0.9699959 |
| Phase-1 RCT-20 | 0.7345679 | 0.6600036 | 0.6959771 | 1.1482551 | 1.1761527 | 0.8166759 | 0.9655176 |
| Phase-1 RCT-248 | 1.1106832 | 1.3520929 | 1.1893849 | 0.9783784 | 0.9677097 | 0.8774297 | 0.9882807 |
| | GAN | GAN | GAN | GAN | GAN | GEN | GEN |
| Compound-Dose (2) | 50 | 50 | 200 | 200 | 200 | 38 | 38 |
| Animal Number (3) | 2442 | 2443 | 2451 | 2452 | 2453 | 221 | 222 |
| Liver Toxicity Necrosis Classification (4) | no | no | no | no | no | no | no |
| Gene Name (5) | | | | | | | |
| Gadd153 | 0.9014682 | 0.843106 | 1.1492279 | 0.9373988 | 0.9648556 | 1.0703622 | 1.1455681 |
| Interferon related developmental regulator | 1.3728855 | 1.4572014 | 1.7453324 | 1.6384251 | 1.6405149 | 0.9672436 | 1.1390474 |
| IFRD1 (PC4) | | | | | | | |
| ID-1 | 0.9344038 | 0.9495077 | 0.9836376 | 0.7812493 | 1.1784389 | 0.9860014 | 0.9640462 |
| Insulin-like growth factor binding protein 1 | 0.9821955 | 1.0208721 | 1.1879667 | 1.2374921 | 0.9173954 | 1.822697 | 1.1452451 |
| NIPK | 1.0079533 | 1.0396165 | 1.055413 | 1.1409038 | 1.1083313 | 0.9591495 | 1.1009685 |
| Phase-1 RCT-50 | 1.0494449 | 1.0434613 | 1.0373479 | 1.0559881 | 1.1551926 | 0.9854285 | 1.1359861 |
| Sarcoplasmic reticulum calcium ATPase | 0.8605072 | 0.8976755 | 0.9459211 | 1.0267525 | 1.1406034 | 0.934634 | 1.2284557 |
| Phase-1 RCT-207 | 0.9070539 | 1.0003488 | 1.1478363 | 1.1171297 | 1.1462348 | 0.9618257 | 0.9608116 |
| Acetyl-CoA carboxylase | 0.8612872 | 0.9502638 | 0.9168768 | 0.960213 | 0.9744401 | 0.8176953 | 0.8269811 |
| Cathepsin L, sequence 2 | 1.1881638 | 1.1985086 | 1.3905112 | 1.6897217 | 1.6290381 | 1.3454994 | 0.796142 |
| Glucokinase | 0.6991381 | 0.7005901 | 0.8022448 | 0.576179 | 0.5008628 | 0.8115969 | 0.7652976 |
| Gadd45 | 1.008709 | 0.9582183 | 1.2484161 | 1.1421276 | 1.1941953 | 1.4829526 | 1.0528592 |
| Phase-1 RCT-18 | 0.8976408 | 0.878736 | 0.8876132 | 0.879046 | 0.9348332 | 0.8830884 | 0.9634497 |
| 3-beta-hydroxysteroid dehydrogenase | 1.475288 | 1.1508926 | 1.2062666 | 1.1929561 | 1.0886662 | 1.311926 | 1.130972 |
| (HSD3B1) | | | | | | | |
| Phase-1 RCT-221 | 0.8195969 | 0.8349316 | 0.8204901 | 0.9022673 | 1.05961 | 0.8342958 | 0.6786928 |
| Heme oxygenase | 1.10049 | 0.9722828 | 1.555585 | 1.4655039 | 2.0376222 | 1.0089239 | 1.0362954 |
| Cyclin dependent kinase 4 | 1.1668346 | 1.0217165 | 1.177608 | 1.340486 | 1.05742 | 1.1086526 | 1.0910149 |
| Phase-1 RCT-75 | 1.0243926 | 0.9869852 | 1.1760796 | 1.3787688 | 1.2327306 | 0.9985637 | 0.9315457 |
| Phase-1 RCT-65 | 0.9084083 | 0.9454216 | 0.8818849 | 0.8619919 | 1.0566046 | 0.8392362 | 0.8631513 |
| Cytochrome P450 1A1 | 1.1309646 | 1.0827435 | 1.4592625 | 2.8689377 | 1.172641 | 0.9047245 | 1.1676958 |
| Peroxisomal multifunctional enzyme type II | 1.3456603 | 1.4140025 | 1.2194929 | 1.4941648 | 1.2578622 | 0.9002509 | 0.8465017 |
| DNA topoisomerase I | 1.1535813 | 1.1415415 | 1.4156852 | 1.3269649 | 1.2347972 | 1.2059957 | 1.2200465 |
| Focal adhesion kinase (pp125FAK) | 1.1723814 | 1.0313327 | 1.4241583 | 1.1322945 | 1.2997657 | 0.9846581 | 1.2716358 |
| NGF-inducible anti-proliferative putative | 1.10827 | 1.0217746 | 1.0031441 | 1.3210475 | 1.1039287 | 1.1873313 | 0.9874322 |
| secreted protein (PC3) | | | | | | | |
| Phase-1 RCT-82 | 0.8801953 | 0.9642041 | 0.9125179 | 0.894482 | 0.9890084 | 0.9064721 | 1.1050645 |
| Senescence marker protein-30 | 1.0598552 | 1.0853969 | 0.9286421 | 1.0624695 | 1.1177529 | 1.033622 | 0.9136775 |
| Melanoma-associated antigen ME491 | 1.0266176 | 0.9307991 | 1.1014501 | 1.0640708 | 1.1231495 | 1.1226124 | 1.0491755 |
| Activating transcription factor 3 | 0.8528027 | 0.8272698 | 0.8103424 | 0.7273536 | 0.7569243 | 0.9305249 | 1.0590037 |
| Tryptophan hydroxylase | 1.0428178 | 0.9539651 | 0.9753123 | 0.9637891 | 0.8697577 | 1.2023127 | 0.9357161 |
| Zinc finger protein | 1.0492547 | 1.0494119 | 0.8034117 | 1.1754973 | 0.9193807 | 0.8431798 | 0.8983616 |
| c-myc | 1.0278081 | 0.9171689 | 1.148303 | 1.0157576 | 0.9838143 | 1.0466344 | 1.3003286 |
| Cholesterol 7-alpha-hydroxylase P450 | 0.9157249 | 0.9763938 | 0.9578473 | 0.761067 | 0.8111154 | 1.1229935 | 1.8491255 |
| VII) | | | | | | | |
| Ferritin H-chain | 1.1819623 | 1.2430978 | 1.0492101 | 1.2814943 | 0.9337964 | 1.178692 | 0.6758007 |
| Phase-1 RCT-197 | 1.0739248 | 1.1294012 | 1.4902544 | 1.1314183 | 1.3803396 | 0.9132541 | 1.0375327 |
| Phase-1 RCT-179 | 1.152815 | 1.1821909 | 1.1555505 | 1.7790123 | 1.8047799 | 1.2418946 | 1.033681 |
| Phase-1 RCT-15 | 1.1224626 | 1.1837616 | 1.208211 | 1.1548798 | 1.3677597 | 1.1249108 | 0.9284031 |
| Argininosuccinate lyase | 1.447407 | 1.3660539 | 1.2777408 | 1.6356899 | 1.5070518 | 1.5717558 | 0.891384 |
| Phase-1 RCT-49 | 0.89801 | 0.9668466 | 1.0112478 | 1.2325155 | 1.4077051 | 1.0566196 | 1.2930865 |
| Carbonic anhydrase III | 0.6652724 | 0.6307227 | 0.3682804 | 0.4471907 | 0.3804785 | 0.6714267 | 0.5746476 |
| Proliferating cell nuclear antigen gene | 0.8464456 | 0.8530536 | 0.946319 | 0.7571231 | 0.8173716 | 1.1574932 | 1.3910277 |
| Integrin beta1 | 1.0878925 | 1.0542861 | 1.408833 | 1.0538571 | 1.20195 | 1.44134 | 1.1583565 |
| Caspase 6 | 1.038705 | 0.9816207 | 1.1290275 | 1.0224676 | 0.9511649 | 1.0793415 | 1.0975618 |
| Phase-1 RCT-116 | 1.064776 | 1.2469491 | 1.5756956 | 1.3616754 | 1.4918815 | 1.171592 | 0.7865976 |
| Phase-1 RCT-109 | 1.0461495 | 1.1004812 | 1.4497476 | 1.4730349 | 1.442 | 1.2715892 | 0.8716604 |
| Phase-1 RCT-71 | 1.000265 | 1.17492 | 1.1093224 | 1.2201664 | 1.4817358 | 0.9674234 | 1.0929259 |
| Elongation factor-1 alpha | 1.0555756 | 1.0804625 | 1.3602378 | 1.4940584 | 1.328736 | 1.1254094 | 0.8172427 |
| Phase-1 RCT-169 | 1.0499644 | 0.9608049 | 1.0792878 | 0.9015466 | 1.3461344 | 0.8636676 | 0.9321175 |
| Pyruvate kinase, muscle | 0.9979615 | 0.9791766 | 1.0995656 | 1.0164696 | 1.0807217 | 1.1199754 | 0.9193481 |
| Nucleosome assembly protein | 0.852124 | 1.3688782 | 0.6739404 | 0.7465932 | 0.8569593 | 0.6479461 | 1.1386935 |
| Phase-1 RCT-127 | 1.0791448 | 1.0488182 | 1.1240592 | 1.5450443 | 1.8532118 | 1.0817263 | 1.1595663 |
| Iron-responsive element-binding protein | 0.980006 | 1.0596461 | 1 | 0.8653559 | 0.866385 | 0.976154 | 0.9634391 |
| Phase-1 RCT-72 | 0.9037745 | 0.9709576 | 0.8898485 | 0.8817892 | 1.1665903 | 0.8299567 | 1.0890157 |
| Phase-1 RCT-144 | 0.9050837 | 0.9585745 | 1.2433691 | 1.1637335 | 1.4817443 | 1.0024972 | 1.0251063 |
| Phase-1 RCT-242 | 0.8678098 | 0.9119036 | 1.26231 | 1.2087405 | 1.2238438 | 0.8708353 | 1.1412474 |
| 17-beta hydroxysteroid dehydrogenase, | 0.7980703 | 1.5703495 | 0.6583799 | 0.4892898 | 0.5271972 | 0.5465289 | 1.1244538 |
| type 2 | | | | | | | |
| Phase-1 RCT-70 | 0.8554208 | 0.8957057 | 0.8011496 | 0.8943167 | 0.9353152 | 0.9007324 | 0.8082652 |
| Ribosomal protein L13A | 1.0558317 | 0.9799271 | 1.3410084 | 1.3350573 | 1.3796122 | 1.4205028 | 0.977382 |
| Cytochrome P450 2E1 | 1.0767244 | 0.7984472 | 0.771584 | 0.9480705 | 0.7624569 | 1.0371962 | 0.9042823 |
| AT-3 | 1.0898496 | 1.0186012 | 1.0275601 | 0.9543699 | 1.0331197 | 0.9963478 | 1.0832655 |
| Phase-1 RCT-270 | 0.8962595 | 0.9053668 | 0.7447391 | 0.6479213 | 0.5758236 | 0.8027597 | 0.861032 |
| Phase-1 RCT-123 | 0.9779226 | 1.1099734 | 0.911158 | 0.9610543 | 0.9392809 | 1.009741 | 1.156317 |
| Alpha-2-macroglobulin, sequence 2 | 1.3695967 | 1.1330676 | 1.4530154 | 1.4572407 | 1.5766407 | 1.1387395 | 1.2709306 |
| Matrix metalloproteinase-1 | 1.1596609 | 1.0747011 | 1.0295709 | 1.1482861 | 0.8304421 | 1.1764649 | 0.6990032 |
| Beta-tubulin, class I | 0.8545832 | 1.1169184 | 1.3057603 | 1.1323972 | 1.4558687 | 1.6597817 | 0.6938647 |
| Phase-1 RCT-161 | 0.8119298 | 0.727452 | 0.5860325 | 0.6434246 | 0.5747805 | 0.7021921 | 0.8276614 |
| 14-3-3 zeta | 1.0619445 | 1 | 1.1699277 | 1.2669055 | 1.3241378 | 1.0677041 | 0.7769265 |
| Stem cell factor | 1.0174489 | 1.0020477 | 0.9965434 | 0.9501237 | 0.9443725 | 0.9825909 | 1.0026604 |
| Macrophage inflammatory protein-2 alpha | 1.1493648 | 0.9138092 | 1.5790453 | 1.6157172 | 1.699052 | 1.4845176 | 1.152255 |
| Thymidylate synthase | 0.9185802 | 0.9697653 | 0.9248511 | 1.0629692 | 0.9206955 | 1.210347 | 1.5095093 |
| c-jun | 0.9708573 | 0.8529849 | 1.0392345 | 0.931391 | 0.9622765 | 1.1030993 | 1.0034012 |
| Superoxide dismutase Mn | 1.002761 | 1.0239928 | 1.0910516 | 0.8741348 | 1.2753716 | 1.5496131 | 1.1650572 |
| Phase-1 RCT-73 | 1.1803197 | 1.1701039 | 1.0579369 | 1.1223853 | 1.1442199 | 1.0912493 | 1.0165731 |
| Macrophage inflammatory protein-1 alpha | 1.0115021 | 1.0619075 | 1.0567342 | 1.0160909 | 1.0054972 | 1.1593976 | 1.3769805 |
| Phase-1 RCT-214 | 1.0194654 | 0.9559037 | 0.8841123 | 0.7885088 | 0.901497 | 1.1836127 | 0.845372 |
| NADH-cytochrome b5 reductase | 0.8935689 | 1.068101 | 0.7756349 | 0.7627271 | 0.6103624 | 0.8503938 | 0.7120092 |
| Interleukin-1 beta | 1.0878898 | 1.0528835 | 1.0225699 | 1.0726529 | 1.059774 | 1.1678648 | 0.7707684 |
| NADP-dependent isocitrate | 1.0169761 | 1.9351695 | 0.7778244 | 0.6837982 | 0.7140889 | 1.1624864 | 0.821252 |
| dehydrogenase, cytosolic | | | | | | | |
| Phase-1 RCT-289 | 0.9935023 | 0.8607413 | 0.7916843 | 0.758639 | 0.7036869 | 1.0019181 | 0.9418685 |
| Glutamine synthetase | 1.1030624 | 0.8870734 | 1.0882293 | 1.2687559 | 1.3409914 | 0.9251174 | 0.8540685 |
| Phase-1 RCT-182 | 0.9812712 | 0.9193443 | 0.8096126 | 0.7726845 | 0.6896624 | 0.7186713 | 0.775637 |
| Ubiquitin conjugating enzyme (RAD 6 | 1.0921634 | 1.145659 | 1.2883147 | 1.9207493 | 1.965763 | 1.0374839 | 0.9973732 |
| homologue) | | | | | | | |
| Extracellular-signal-regulated kinase 1 | 0.9689914 | 0.8544432 | 0.8785737 | 0.9012746 | 0.84091 | 0.7368435 | 0.6449138 |
| Emerin | 0.889564 | 1.0037388 | 0.748053 | 1.1839305 | 1.0158272 | 1.0136006 | 1.0416571 |
| Phase-1 RCT-78 | 1.0668236 | 1.0020108 | 0.8235177 | 0.8565171 | 0.8865965 | 0.8127312 | 0.8994569 |
| Phase-1 RCT-212 | 0.9653602 | 0.9621484 | 1.0996416 | 1.0374302 | 1.1488208 | 1.0301659 | 0.9931938 |
| Phase-1 RCT-168 | 0.8774785 | 0.846267 | 0.9023772 | 0.5943256 | 0.657544 | 0.9290131 | 0.8309328 |
| Lysyl hydroxylase | 1.0253655 | 0.8819727 | 1.2858815 | 0.9512619 | 1.086577 | 0.9095359 | 0.9471982 |
| Phase-1 RCT-55 | 0.7535167 | 0.80416 | 1.2821794 | 0.7700851 | 0.959801 | 1.1788757 | 0.8124654 |
| MHC class I antigen RT1.A1(f) alpha- | 0.9129567 | 0.9804375 | 1.1343632 | 0.9999999 | 1.0471803 | 1.2995924 | 0.8518928 |
| chain | | | | | | | |
| Phase-1 RCT-40 | 1.113898 | 1.1622858 | 1.042037 | 1.0697123 | 0.9016268 | 1.0200598 | 0.8751528 |
| Cyclin G | 1.0076786 | 1.12747 | 1.2449757 | 1.5388645 | 1.7349867 | 1.2107111 | 1.1499631 |
| Peroxisome proliferator activated receptor | 0.8973623 | 0.9329214 | 0.855311 | 0.7240531 | 0.7215856 | 0.9514801 | 1.1842991 |
| gamma | | | | | | | |
| Phase-1 RCT-280 | 1.3210025 | 1.157926 | 1.0182133 | 1.1396981 | 1.1539787 | 1.0942652 | 0.9970065 |
| D-dopachrome tautomerase | 1.1717098 | 1.1267455 | 0.8710994 | 0.9498455 | 0.8754229 | 1.0387434 | 0.6916322 |
| Phase-1 RCT-287 | 1.1107905 | 1.0890795 | 1.0862077 | 1.1337279 | 1.1347337 | 0.9354578 | 0.8113872 |
| Phase-1 RCT-119 | 1.1296128 | 0.9059465 | 0.714914 | 0.9636937 | 0.985486 | 0.9205625 | 0.9346064 |
| Thioredoxin-1 (Trx1) | 1.0181463 | 1.3146865 | 1.1915324 | 1.3072066 | 1.3707068 | 1.3778244 | 1.1798495 |
| Phase-1 RCT-225 | 0.963665 | 0.8695242 | 0.9913808 | 0.8092046 | 0.8623826 | 1.2118 | 0.8340029 |
| Protein kinase C alpha | 0.9030824 | 0.8930288 | 1.0734869 | 0.9120306 | 0.906357 | 1.0593499 | 0.8852184 |
| Phase-1 RCT-87 | 0.9402058 | 1.1282787 | 1.0383265 | 0.89797 | 0.9964948 | 0.9542386 | 1.08826 |
| Phase-1 RCT-59 | 0.943804 | 1.0034841 | 1.1245518 | 1.1838737 | 1.2408333 | 1.0570716 | 1.0216776 |
| C4b-binding protein | 1.4672484 | 1.1691604 | 1.3317046 | 2.1333423 | 1.8200132 | 1.0165676 | 1.060791 |
| Phase-1 RCT-204 | 1.1159635 | 1.2148981 | 0.9930807 | 1.2374928 | 1.2162837 | 0.9422561 | 1.0262543 |
| Carnitine palmitoyl-CoA transferase | 0.8503996 | 0.8530975 | 1.0635327 | 1.0335853 | 1.1649772 | 0.8821307 | 1.0210856 |
| 8-oxoguanine DNA glycosylase | 0.8958565 | 0.8519062 | 0.9961863 | 0.9527473 | 0.9175338 | 1.1316018 | 1.1515138 |
| H-rev107 | 1.060363 | 0.9478003 | 1.0263116 | 1.0076557 | 0.923641 | 1.1491742 | 1.2778827 |
| Preproalbumin | 1.5025563 | 1.4692361 | 0.8719291 | 1.4748536 | 1.0783895 | 1.2096382 | 0.8665234 |
| Phase-1 RCT-177 | 1.3011819 | 1.2690438 | 0.9920601 | 1.1864476 | 1.2811844 | 1.1455138 | 1.0185932 |
| Phase-1 RCT-110 | 0.9433305 | 0.8891842 | 0.8450171 | 0.829705 | 0.9248722 | 0.8903044 | 1.0983253 |
| Phase-1 RCT-227 | 1.2043598 | 1.1536828 | 0.8755224 | 0.9408242 | 1.0213375 | 0.8028659 | 0.8853698 |
| Neuropeptide Y | 1.0134827 | 0.9492587 | 0.9556651 | 0.964656 | 1.004993 | 0.9280958 | 1.0240383 |
| Phase-1 RCT-277 | 1.135705 | 0.9490067 | 1.0512284 | 1.1536216 | 0.9399878 | 1.5588206 | 1.1858062 |
| Phase-1 RCT-229 | 0.9113848 | 0.8639784 | 0.8426505 | 0.8397842 | 0.9842938 | 0.780215 | 1.1164922 |
| Phase-1 RCT-34 | 0.9470553 | 1.0355276 | 1.0917329 | 0.7194856 | 0.8659429 | 1.0692991 | 0.9373187 |
| Choline kinase | 0.984596 | 0.9739658 | 0.9674714 | 0.9379691 | 0.9330737 | 1.2603903 | 1.3305864 |
| Phase-1 RCT-20 | 0.9753729 | 1.0549905 | 1.1248496 | 1.1060641 | 1.1518753 | 0.8523327 | 1.2000538 |
| Phase-1 RCT-248 | 0.9179393 | 0.985334 | 1.1071438 | 0.9714993 | 0.9888589 | 1.099086 | 0.9118907 |
| | GEN | GEN | GEN | GEN | HYD | HYD | HYD |
| Compound-Dose (2) | 38 | 150 | 150 | 150 | 250 | 250 | 250 |
| Animal Number (3) | 223 | 231 | 232 | 233 | 1221 | 1222 | 1223 |
| Liver Toxicity Necrosis Classification (4) | no | no | no | no | no | no | no |
| Gene Name (5) | | | | | | | |
| Gadd153 | 1.116924 | 1.0480762 | 1.1497957 | 0.9955874 | 1.0137774 | 1.0955021 | 1.2753471 |
| Interferon related developmental regulator | 0.9935949 | 1.0385746 | 0.935793 | 0.8920229 | 0.6920494 | 0.7604014 | 0.7968494 |
| IFRD1 (PC4) | | | | | | | |
| ID-1 | 0.8951258 | 1.1923246 | 0.9967165 | 1.0135331 | 1.1734383 | 1.1754112 | 1.1274909 |
| Insulin-like growth factor binding protein 1 | 1.4183425 | 1.0239638 | 1.0873212 | 0.9726707 | 1.2437371 | 0.995615 | 0.9317171 |
| NIPK | 0.9697488 | 0.974451 | 0.9496436 | 0.975917 | 1.4792056 | 1.1551942 | 1.1605475 |
| Phase-1 RCT-50 | 1.0054073 | 1.5311438 | 1.0865055 | 0.9632653 | 1.4154121 | 1.3493745 | 1.3052951 |
| Sarcoplasmic reticulum calcium ATPase | 1.196705 | 1.2235627 | 1.201415 | 0.9438598 | 0.4513507 | 0.6402129 | 0.6615394 |
| Phase-1 RCT-207 | 1.0585532 | 1.2186716 | 1.3824451 | 0.8919919 | 1.3056359 | 1.2389787 | 1.2956556 |
| Acetyl-CoA carboxylase | 0.8656976 | 1.1247956 | 0.9208205 | 0.8700843 | 0.9285855 | 1.1893504 | 1.2015167 |
| Cathepsin L, sequence 2 | 1.1158152 | 0.8537839 | 0.9143055 | 1.1194373 | 0.8249791 | 0.8105725 | 0.6063736 |
| Glucokinase | 0.9040308 | 1.1316848 | 0.8982336 | 0.8436323 | 0.5609455 | 0.8398128 | 1.1654927 |
| Gadd45 | 0.9981652 | 1.4607611 | 1.0111365 | 1.0017478 | 1.3256884 | 1.299629 | 1.342712 |
| Phase-1 RCT-18 | 0.847479 | 1.2198851 | 0.9454225 | 0.9683269 | 1.0709858 | 1.1367725 | 1.0665774 |
| 3-beta-hydroxysteroid dehydrogenase | 1.3011721 | 0.9952771 | 1.2317649 | 1.3482411 | 2.3129058 | 2.0049672 | 1.8091785 |
| (HSD3B1) | | | | | | | |
| Phase-1 RCT-221 | 0.6357262 | 0.7086809 | 0.7007161 | 0.8560172 | 0.9452898 | 0.8274027 | 0.6891304 |
| Heme oxygenase | 0.938485 | 1.1608096 | 0.8930439 | 1.2440006 | 1.1396204 | 0.9953123 | 0.9568135 |
| Cyclin dependent kinase 4 | 1.2730134 | 1.1614503 | 1.1420301 | 0.9626421 | 1.8712351 | 1.4995345 | 1.7810838 |
| Phase-1 RCT-75 | 0.9070368 | 0.9875463 | 0.985825 | 0.9916016 | 1.3885722 | 0.9898075 | 0.8976518 |
| Phase-1 RCT-65 | 0.81102 | 0.8813787 | 0.8676654 | 1.0266972 | 2.0425692 | 1.62572 | 1.7619551 |
| Cytochrome P450 1A1 | 0.990917 | 1.4776978 | 1.0538638 | 0.9687082 | 1.2154857 | 2.176667 | 2.058543 |
| Peroxisomal multifunctional enzyme type II | 0.9376046 | 0.7264782 | 0.8870834 | 1.0063287 | 2.671572 | 1.4174465 | 0.8539686 |
| DNA topoisomerase I | 0.9284384 | 0.6882712 | 0.8547917 | 0.9788873 | 1.4643803 | 1.2968099 | 0.6775973 |
| Focal adhesion kinase (pp125FAK) | 0.9947648 | 1.5139595 | 1.055058 | 0.9004285 | 0.6071912 | 0.728883 | 1.0059913 |
| NGF-inducible anti-proliferative putative | 1.0159398 | 1.2538036 | 0.9814974 | 1.1364367 | 0.882414 | 0.9635267 | 0.9289117 |
| secreted protein (PC3) | | | | | | | |
| Phase-1 RCT-82 | 0.9476677 | 1.523524 | 0.9420185 | 0.9070449 | 0.7025895 | 0.7952433 | 1.1746652 |
| Senescence marker protein-30 | 1.0184888 | 0.6706753 | 1.0950288 | 1.1406283 | 0.6554651 | 0.6583819 | 0.3775666 |
| Melanoma-associated antigen ME491 | 1.0089228 | 1.1242079 | 0.9229324 | 1.0268782 | 0.7845197 | 0.8659594 | 0.9788584 |
| Activating transcription factor 3 | 0.8964248 | 0.8424462 | 1.2477546 | 0.9785423 | 1.4580142 | 1.898293 | 1.2958541 |
| Tryptophan hydroxylase | 0.8899578 | 0.6824432 | 0.8118462 | 1.1462892 | 2.4542472 | 2.410999 | 1.42548 |
| Zinc finger protein | 0.905862 | 0.6938676 | 0.935887 | 0.9499076 | 1.3983254 | 1.4658945 | 1.0178479 |
| c-myc | 1.0182568 | 1.463417 | 1.0473427 | 0.9852383 | 1.3697301 | 1.2445552 | 1.3963808 |
| Cholesterol 7-alpha-hydroxylase P450 | 1.0982882 | 2.0509653 | 1.250733 | 0.9713134 | 0.5313051 | 1.4202547 | 1.7720687 |
| VII) | | | | | | | |
| Ferritin H-chain | 1.2925658 | 0.78948 | 0.9509149 | 1.240332 | 0.6093143 | 0.5511762 | 0.6058133 |
| Phase-1 RCT-197 | 0.9679097 | 0.9038767 | 1.0173031 | 0.9612361 | 0.7638151 | 0.7941616 | 0.9920073 |
| Phase-1 RCT-179 | 1.0984774 | 1.0714421 | 1.1396568 | 1.1858338 | 1.1373905 | 0.8716317 | 0.8263084 |
| Phase-1 RCT-15 | 1.2165934 | 0.8420305 | 1.1084207 | 0.9602706 | 1.2607985 | 1.4797299 | 1.4840046 |
| Argininosuccinate lyase | 1.1402718 | 0.901285 | 0.9888495 | 0.9198576 | 4.468011 | 1.656183 | 0.9638739 |
| Phase-1 RCT-49 | 1.0019228 | 3.8708284 | 1.0704329 | 1.1209397 | 0.8506332 | 0.8910079 | 1.1023805 |
| Carbonic anhydrase III | 1.1296297 | 0.7115365 | 1.2608541 | 0.9141739 | 1.9664334 | 1.1512041 | 1.5677643 |
| Proliferating cell nuclear antigen gene | 1.0080376 | 1.4032357 | 1.1798093 | 1.075755 | 0.5614929 | 0.7162985 | 1.4706718 |
| Integrin beta1 | 1.2839816 | 1.2601422 | 1.146219 | 0.9881821 | 1.4596668 | 0.9735369 | 1.0025735 |
| Caspase 6 | 1.1483971 | 1.2365146 | 1.0581696 | 1 | 0.9994767 | 0.9488566 | 0.9143029 |
| Phase-1 RCT-116 | 1.0958657 | 0.9890918 | 0.9989336 | 0.8848883 | 3.227019 | 1.9185466 | 1.9347473 |
| Phase-1 RCT-109 | 1.0551664 | 1.0914787 | 0.975318 | 1.0201967 | 0.8590411 | 0.7175077 | 0.6996204 |
| Phase-1 RCT-71 | 0.996393 | 1.262195 | 1.0176653 | 0.9243626 | 1.2538561 | 1.320946 | 1.2090969 |
| Elongation factor-1 alpha | 0.8605086 | 0.6405158 | 0.7723705 | 0.8664582 | 0.7571666 | 0.7529839 | 0.7755522 |
| Phase-1 RCT-169 | 0.8008957 | 1.0593635 | 0.9709875 | 1.01172 | 0.6980453 | 1.0753379 | 1.061102 |
| Pyruvate kinase, muscle | 1.0489587 | 0.9826822 | 0.9473725 | 0.8946341 | 1.554801 | 1.3950188 | 1.3150873 |
| Nucleosome assembly protein | 1.3161695 | 0.7461773 | 1.0809835 | 0.9666243 | 1.1450367 | 1.3034613 | 1.0062547 |
| Phase-1 RCT-127 | 1.1620669 | 1.2713369 | 1.08586 | 1.0188742 | 0.8818069 | 0.9067317 | 1.0234084 |
| Iron-responsive element-binding protein | 0.9808629 | 0.9341191 | 0.9849295 | 1.0105636 | 0.8111386 | 0.743988 | 0.777966 |
| Phase-1 RCT-72 | 0.8276373 | 1.2357693 | 1.0064553 | 1.0093513 | 0.8626939 | 1.0220395 | 1.0221149 |
| Phase-1 RCT-144 | 0.9793329 | 1.3141469 | 0.9733074 | 0.9406199 | 0.7486181 | 0.7751261 | 1.0344529 |
| Phase-1 RCT-242 | 1.0450768 | 1.3873559 | 1.0756491 | 0.8410632 | 0.9280387 | 1.0941325 | 1.3387752 |
| 17-beta hydroxysteroid dehydrogenase, | 1.4487206 | 0.6969976 | 1.1555077 | 1.1101123 | 2.128221 | 1.9304622 | 1.3256708 |
| type 2 | | | | | | | |
| Phase-1 RCT-70 | 0.841387 | 0.9712986 | 0.8598809 | 0.9661358 | 1.0973467 | 1.0600516 | 1.0585148 |
| Ribosomal protein L13A | 0.9960434 | 1.1315813 | 1.0419748 | 1.0440089 | 0.8615068 | 0.7143555 | 0.6908701 |
| Cytochrome P450 2E1 | 1.0464361 | 0.9438023 | 1.047321 | 0.8860287 | 1.0001858 | 0.7147515 | 0.9569551 |
| AT-3 | 0.9144049 | 1.4297458 | 0.9747766 | 1.1775764 | 1.1092932 | 1.1519599 | 0.8723936 |
| Phase-1 RCT-270 | 0.8250377 | 0.6976289 | 0.8398075 | 0.8747118 | 0.896094 | 0.8447059 | 0.7932652 |
| Phase-1 RCT-123 | 0.9588804 | 1.3994205 | 1.0618527 | 0.9903233 | 1.076341 | 1.0188893 | 1.0781587 |
| Alpha-2-macroglobulin, sequence 2 | 1.2446979 | 1.0256999 | 1.1155715 | 1.0054308 | 0.9677621 | 1.1435027 | 1.1852075 |
| Matrix metalloproteinase-1 | 1.2222726 | 0.7990318 | 0.9520495 | 0.8625777 | 1.0034385 | 0.8528717 | 0.8125105 |
| Beta-tubulin, class I | 0.9549193 | 1.4322449 | 0.855743 | 1.0843277 | 2.4807625 | 1.4334477 | 1.4266701 |
| Phase-1 RCT-161 | 0.959707 | 1.1610465 | 0.9934688 | 0.9541333 | 2.1768107 | 1.7720755 | 1.8269267 |
| 14-3-3 zeta | 0.9673036 | 1.2611899 | 1.3101892 | 1.0822511 | 2.355444 | 1.5731219 | 1.1701683 |
| Stem cell factor | 1.134728 | 0.9799763 | 1.0488839 | 0.9218944 | 1.127844 | 0.9231771 | 0.9702274 |
| Macrophage inflammatory protein-2 alpha | 1.3557 | 0.9761956 | 1.1075238 | 0.9382888 | 1.2998276 | 1.2100937 | 1.0129354 |
| Thymidylate synthase | 1.1346177 | 1.951673 | 1.3067101 | 0.9717909 | 1.0342925 | 0.9720078 | 1.101407 |
| c-jun | 0.9775224 | 1.085403 | 0.9639383 | 0.8820633 | 1.3901203 | 1.1260499 | 1.3708405 |
| Superoxide dismutase Mn | 1.0460523 | 1.2479638 | 1.092292 | 1.238414 | 0.9719535 | 0.8542314 | 1.1019026 |
| Phase-1 RCT-73 | 1.0230572 | 0.9782122 | 1.0083344 | 1.051329 | 1.386829 | 1.4485353 | 1.3728833 |
| Macrophage inflammatory protein-1 alpha | 1.1824238 | 1.6834683 | 1.5444982 | 0.802558 | 1.0252994 | 1.3744569 | 1.1819838 |
| Phase-1 RCT-214 | 1.0078163 | 1.1546727 | 1.3170553 | 1.2458215 | 1.1651486 | 1.2109371 | 0.8906194 |
| NADH-cytochrome b5 reductase | 0.7826652 | 0.7490249 | 0.9205517 | 0.8387539 | 1.1892797 | 0.8283449 | 0.7765053 |
| Interleukin-1 beta | 1.2068555 | 0.8212215 | 0.9703908 | 0.9973503 | 0.8670333 | 0.9595634 | 1.0292934 |
| NADP-dependent isocitrate | 0.9701836 | 0.8823232 | 0.918551 | 1.3085232 | 0.8422471 | 0.7923732 | 0.5453488 |
| dehydrogenase, cytosolic | | | | | | | |
| Phase-1 RCT-289 | 1.1400585 | 0.8869867 | 1.0105634 | 1.019051 | 1.1103321 | 1.007298 | 0.8544939 |
| Glutamine synthetase | 0.9724679 | 0.6863884 | 0.842947 | 1.049093 | 0.8964816 | 0.8208523 | 0.9865772 |
| Phase-1 RCT-182 | 0.8366676 | 0.7772906 | 0.8851211 | 0.9321982 | 0.6896408 | 0.6327688 | 0.7115637 |
| Ubiquitin conjugating enzyme (RAD 6 | 1.0963092 | 0.968056 | 1.1322113 | 1.1181896 | 0.8458849 | 0.8643935 | 0.7036774 |
| homologue) | | | | | | | |
| Extracellular-signal-regulated kinase 1 | 1.1172776 | 0.8193423 | 1.0973734 | 0.9687206 | 1.7028501 | 1.3313763 | 1.8639518 |
| Emerin | 1.0754925 | 1.1337421 | 1.2379216 | 0.923813 | 1.3782345 | 1.2376901 | 1.2277554 |
| Phase-1 RCT-78 | 0.9807785 | 0.9295158 | 0.9715889 | 0.9506783 | 1.1702471 | 1.1030843 | 1.039241 |
| Phase-1 RCT-212 | 0.8807353 | 1.114313 | 0.916162 | 0.9363765 | 1.0870261 | 1.0778021 | 1.1176935 |
| Phase-1 RCT-168 | 0.8207086 | 0.6704248 | 0.8131917 | 0.995487 | 0.8585935 | 0.6962553 | 0.5320558 |
| Lysyl hydroxylase | 0.9753053 | 0.7047661 | 0.8949256 | 1.01154 | 0.9192871 | 0.7642734 | 0.9625465 |
| Phase-1 RCT-55 | 0.6484741 | 0.8285705 | 0.8182316 | 0.9853105 | 0.7975545 | 1.0717057 | 1.2100164 |
| MHC class I antigen RT1.A1(f) alpha- | 1.1186316 | 1.0644019 | 1.0027851 | 1.0012399 | 2.6169622 | 2.57511 | 2.2506177 |
| chain | | | | | | | |
| Phase-1 RCT-40 | 0.9152761 | 0.826957 | 0.8324946 | 1.0196509 | 0.7338942 | 0.7201094 | 0.6242553 |
| Cyclin G | 1.2357011 | 1.2510049 | 1.1727731 | 0.9719053 | 1.3298053 | 1.4268534 | 1.4572672 |
| Peroxisome proliferator activated receptor | 1.0346949 | 2.1819718 | 1.0971878 | 0.9804069 | 1.2769954 | 1.1746961 | 1.4618249 |
| gamma | | | | | | | |
| Phase-1 RCT-280 | 1.1283395 | 0.7555603 | 1.160006 | 0.9255565 | 1.0574163 | 1.281224 | 1.062186 |
| D-dopachrome tautomerase | 0.7882415 | 0.7160934 | 0.8090432 | 1.4854132 | 1.2697219 | 1.0475911 | 0.8089448 |
| Phase-1 RCT-287 | 0.8973974 | 0.9260852 | 0.8938855 | 1.0203444 | 0.7856503 | 0.7270374 | 0.8315586 |
| Phase-1 RCT-119 | 0.9386873 | 0.7203397 | 0.8453419 | 0.9709249 | 0.8009432 | 1.0736276 | 0.9853802 |
| Thioredoxin-1 (Trx1) | 1.199383 | 1.0892138 | 1.11131 | 1.5781049 | 0.7234774 | 0.7304493 | 0.7154251 |
| Phase-1 RCT-225 | 0.8204902 | 0.877703 | 0.7195414 | 0.7711036 | 0.5066119 | 0.5834054 | 0.7241668 |
| Protein kinase C alpha | 0.9228698 | 0.9568267 | 0.9254788 | 0.9184474 | 0.7094191 | 0.7038986 | 1.0521293 |
| Phase-1 RCT-87 | 0.997528 | 1.1143898 | 0.956871 | 0.8913809 | 0.8750656 | 0.8696955 | 1.113316 |
| Phase-1 RCT-59 | 1.156861 | 0.8134198 | 1.1321657 | 0.8170744 | 0.8571282 | 1.2150049 | 1.0701352 |
| C4b-binding protein | 1.181473 | 0.8977435 | 0.8584343 | 1.0176011 | 1.0769876 | 1.2457002 | 1.4125148 |
| Phase-1 RCT-204 | 0.9891626 | 1.0815848 | 0.9683682 | 1.0224518 | 1.1951008 | 1.0011208 | 1.1851948 |
| Carnitine palmitoyl-CoA transferase | 1.1069888 | 1.2401621 | 1.3571017 | 1.1315383 | 2.3173766 | 1.3563243 | 1.5185031 |
| 8-oxoguanine DNA glycosylase | 1.0451355 | 1.5996459 | 1.1466529 | 0.9990694 | 0.9697124 | 0.948754 | 1.1443964 |
| H-rev107 | 0.9680162 | 1.409953 | 1.0996817 | 1.0581121 | 1.0031744 | 1.0118678 | 0.9563499 |
| Preproalbumin | 1.2233015 | 0.7253594 | 0.9939266 | 1.5344672 | 4.598472 | 3.033431 | 3.065234 |
| Phase-1 RCT-177 | 0.9953614 | 0.7474806 | 0.9226621 | 1.3477937 | 1.7448574 | 1.3477274 | 0.7854078 |
| Phase-1 RCT-110 | 1.0485688 | 1.4263303 | 1.1013055 | 0.961236 | 0.9775465 | 1.1534055 | 1.2200861 |
| Phase-1 RCT-227 | 1.010583 | 0.8560627 | 0.9928314 | 1.2171544 | 1.0387074 | 0.9183803 | 0.8986918 |
| Neuropeptide Y | 0.8813534 | 1.5922107 | 0.9479405 | 0.9241517 | 1.1255876 | 1.262041 | 1.2450336 |
| Phase-1 RCT-277 | 0.7828952 | 0.7233312 | 0.5615006 | 1.0852458 | 0.7135744 | 0.7488682 | 0.6179105 |
| Phase-1 RCT-229 | 0.946345 | 1.4116302 | 1.0437288 | 0.9568515 | 0.9821544 | 1.3723329 | 1.4595995 |
| Phase-1 RCT-34 | 1.0821942 | 0.9877677 | 0.9030639 | 0.9353842 | 0.7944045 | 0.8206962 | 0.9668785 |
| Choline kinase | 1.0001342 | 1.3453416 | 1.0942507 | 0.9780589 | 1.3683761 | 1.1744763 | 1.1965885 |
| Phase-1 RCT-20 | 0.9623654 | 1.1525395 | 0.9670355 | 0.931388 | 0.9852194 | 1.2175905 | 1.3754785 |
| Phase-1 RCT-248 | 1.0682054 | 1.209613 | 1.0254188 | 0.9434137 | 0.9846647 | 1.1325886 | 1.1648085 |
| | HYD | HYD | ISON | ISON | ISON | ISON | ISON |
| Compound-Dose (2) | 1000 | 1000 | 50 | 50 | 50 | 200 | 200 |
| Animal Number (3) | 1231 | 1233 | 1941 | 1942 | 1943 | 1951 | 1952 |
| Liver Toxicity Necrosis Classification (4) | no | no | no | no | no | no | no |
| Gene Name (5) | | | | | | | |
| Gadd153 | 1.8127629 | 1.3033437 | 1.2639816 | 1.0275779 | 1.1779433 | 1.0215728 | 1.0858066 |
| Interferon related developmental regulator | 0.9778913 | 0.9150389 | 1.0805734 | 1.0728949 | 1.0235034 | 1.2561146 | 1.0732973 |
| IFRD1 (PC4) | | | | | | | |
| ID-1 | 1.4386284 | 1.2894638 | 1.104685 | 0.9699711 | 0.9389923 | 1 | 1.00917 |
| Insulin-like growth factor binding protein 1 | 0.7283251 | 1.1073048 | 1.6135948 | 1.0007716 | 0.9799231 | 0.9524644 | 1.0436653 |
| NIPK | 1.3261899 | 1.1046464 | 1.0196611 | 1.0976804 | 1.1842904 | 1.1155797 | 1.0779071 |
| Phase-1 RCT-50 | 1.3459699 | 1.1649827 | 1.0318154 | 1.0358231 | 1.0169414 | 1.0932341 | 1.0569249 |
| Sarcoplasmic reticulum calcium ATPase | 0.7151505 | 1.2226083 | 0.9396891 | 1.1481997 | 0.9357179 | 0.967308 | 0.970721 |
| Phase-1 RCT-207 | 1.2607771 | 1.055736 | 1.1944637 | 1.0706694 | 1.0672678 | 1.0348487 | 1.0806477 |
| Acetyl-CoA carboxylase | 0.986511 1 | 0.8605099 | 0.8835453 | 0.9090707 | 0.9521575 | 0.9679577 | 0.8862931 |
| Cathepsin L, sequence 2 | 0.7438993 | 0.9303828 | 1.4262623 | 1.0975043 | 0.9433064 | 0.9081122 | 0.9857665 |
| Glucokinase | 1.2534503 | 1.3458538 | 0.9393693 | 1.0068666 | 1.6297134 | 0.7240624 | 0.5798228 |
| Gadd45 | 1.4860467 | 0.8952192 | 0.9578247 | 1.0938023 | 0.9245344 | 0.9832614 | 0.9691276 |
| Phase-1 RCT-18 | 1.0032573 | 1.0588115 | 0.9098097 | 0.9149553 | 0.9842959 | 0.9288537 | 0.9737144 |
| 3-beta-hydroxysteroid dehydrogenase | 1.8775456 | 1.0488441 | 0.986741 | 0.9841229 | 0.7930035 | 0.8677079 | 0.6761611 |
| (HSD3B1) | | | | | | | |
| Phase-1 RCT-221 | 1.0313028 | 0.7555975 | 0.9259038 | 0.9196265 | 1.0169015 | 0.9191205 | 0.8888701 |
| Heme oxygenase | 0.9866343 | 1.016218 | 1.0609566 | 1.3736643 | 1.00102 | 1.2013853 | 1.0829936 |
| Cyclin dependent kinase 4 | 1.6161106 | 1.0470572 | 0.9955096 | 1.0265752 | 1.0358717 | 0.9902448 | 1.0470725 |
| Phase-1 RCT-75 | 1.0570194 | 0.8881993 | 1.1892524 | 1.0077782 | 0.9610896 | 1.0738553 | 0.989449 |
| Phase-1 RCT-65 | 1.7665886 | 1.0039515 | 1.1961607 | 1.2901063 | 0.9653404 | 1.0963484 | 1.1762989 |
| Cytochrome P450 1A1 | 1.9552969 | 1.4072013 | 0.9802699 | 0.9202403 | 1.2744255 | 1.2827277 | 1.5018231 |
| Peroxisomal multifunctional enzyme type II | 0.9167879 | 0.9481372 | 1.0296814 | 0.8275405 | 0.9039344 | 1.105777 | 1.0666063 |
| DNA topoisomerase I | 0.8594766 | 0.8932182 | 0.8508397 | 1.01601 | 1.0345421 | 0.985731 | 0.9375145 |
| Focal adhesion kinase (pp125FAK) | 0.904579 | 1.0249529 | 0.9327166 | 0.9460297 | 1.0046736 | 0.9989738 | 0.9571227 |
| NGF-inducible anti-proliferative putative | 0.810518 | 0.9549859 | 0.8427872 | 0.8481997 | 0.9395412 | 0.9315586 | 0.993801.9 |
| secreted protein (PC3) | | | | | | | |
| Phase-1 RCT-82 | 0.9143178 | 0.9536931 | 0.9198092 | 0.9835014 | 0.9923338 | 0.9923422 | 0.9907517 |
| Senescence marker protein-30 | 0.4192783 | 0.7915637 | 0.6523272 | 1.0684735 | 0.9785754 | 0.7837327 | 0.6749392 |
| Melanoma-associated antigen ME491 | 0.713054 | 0.7485291 | 1.0081222 | 0.8670077 | 0.96662 | 0.7895012 | 0.8608316 |
| Activating transcription factor 3 | 2.0676463 | 1.3807546 | 1.1472831 | 1.1841124 | 1.4645828 | 1.2486728 | 1.0715251 |
| Tryptophan hydroxylase | 1.5242419 | 1.056615 | 1.0711011 | 1.1535026 | 0.9257048 | 1.0069169 | 1.0325538 |
| Zinc finger protein | 1.0603677 | 1.2716469 | 1.061783 | 1.1132207 | 0.9522577 | 1.103619 | 1.0479739 |
| c-myc | 1.7979863 | 1.3446151 | 1.2089368 | 0.9455189 | 1.1075269 | 1.076363 | 0.9945518 |
| Cholesterol 7-alpha-hydroxylase P450 | 1.0628024 | 0.6332693 | 1.128875 | 0.8503636 | 0.7955671 | 0.9705764 | 0.9450745 |
| VII) | | | | | | | |
| Ferritin H-chain | 0.5375916 | 0.7656556 | 0.9096405 | 0.819886 | 0.8471689 | 0.8785505 | 0.9328309 |
| Phase-1 RCT-197 | 1.089026 | 0.9814068 | 1.1559854 | 1.0394638 | 1.0805802 | 1.0223625 | 1.1421398 |
| Phase-1 RCT-179 | 0.9054433 | 0.8237573 | 1.2473911 | 1.0652453 | 0.9710499 | 0.9449483 | 0.9783624 |
| Phase-1 RCT-15 | 1.7098378 | 1.0844592 | 1.2936307 | 1.114612 | 0.9491843 | 1.0144848 | 1.0737094 |
| Argininosuccinate lyase | 1.325439 | 0.6925623 | 1.640089 | 1.414619 | 1.1419255 | 2.1548169 | 2.541465 |
| Phase-1 RCT-49 | 1.1171913 | 0.9914433 | 0.9915673 | 0.9101881 | 1.0070521 | 1.0446242 | 1.1136217 |
| Carbonic anhydrase III | 0.5416368 | 0.5530762 | 0.4967544 | 1.5571764 | 1.0396795 | 0.7442491 | 0.5995918 |
| Proliferating cell nuclear antigen gene | 1.3428392 | 1.2230747 | 0.9960288 | 0.9176267 | 1.0970017 | 1.0408714 | 1.0058836 |
| Integrin beta1 | 0.6802101 | 0.988685 | 0.9450501 | 0.9800873 | 1.0905957 | 1.0357596 | 1.1580117 |
| Caspase 6 | 0.9648404 | 1.0759934 | 1.057368 | 1.0309072 | 0.9958042 | 1.0467478 | 1.0624574 |
| Phase-1 RCT-116 | 2.616404 | 0.8311365 | 1.6470495 | 1.4503983 | 0.8869743 | 1.2720273 | 1.1770811 |
| Phase-1 RCT-109 | 0.7153949 | 0.7129597 | 1.111306 | 0.977453 | 1.1637768 | 1.006255 | 1.1727868 |
| Phase-1 RCT-71 | 1.0166316 | 0.850418 | 0.9756042 | 0.9651567 | 1.0074083 | 1.1433089 | 1.337439 |
| Elongation factor-1 alpha | 0.8246187 | 0.7350637 | 1.1449325 | 0.966876 | 1.0156159 | 0.9687082 | 0.9316629 |
| Phase-1 RCT-169 | 1.0749652 | 1.3529398 | 0.8543268 | 1.2413396 | 1.0501451 | 3.3783185 | 0.8368303 |
| Pyruvate kinase, muscle | 1.4128987 | 0.9844765 | 1.2129196 | 1.1040856 | 0.9984601 | 1.0816653 | 1.0765383 |
| Nucleosome assembly protein | 0.8269138 | 0.8680569 | 1.0066713 | 1.2414287 | 1.0700731 | 1.0107627 | 0.866955 |
| Phase-1 RCT-127 | 1.1685897 | 1.1069552 | 0.9591553 | 0.9258835 | 0.9441446 | 1.0484929 | 0.9941824 |
| Iron-responsive element-binding protein | 0.8059678 | 0.8437884 | 0.8846899 | 0.8578575 | 0.948365 | 0.9258654 | 0.9833475 |
| Phase-1 RCT-72 | 0.989335 | 1.4632838 | 0.9736293 | 1.1630596 | 1.1051718 | 0.9726219 | 0.966008 |
| Phase-1 RCT-144 | 1.2627819 | 0.9249013 | 1.0280219 | 0.901979 | 0.9716827 | 1.0264837 | 1.1588262 |
| Phase-1 RCT-242 | 1.3238322 | 1.1202866 | 0.9795017 | 0.9618142 | 1.0016005 | 0.9794442 | 0.9947801 |
| 17-beta hydroxysteroid dehydrogenase, | 0.5787742 | 0.3567661 | 1.2255769 | 2.138751 | 1.0666524 | 0.9169991 | 0.9558621 |
| type 2 | | | | | | | |
| Phase-1 RCT-70 | 1.0415297 | 0.9781556 | 1.0180413 | 0.9640293 | 1.0420454 | 1.1022962 | 1.1678461 |
| Ribosomal protein L13A | 0.9789132 | 0.7276442 | 1.2446029 | 0.9968991 | 1.1772531 | 0.9422759 | 1.0867789 |
| Cytochrome P450 2E1 | 0.7966334 | 1.0952439 | 0.952735 | 0.7793698 | 0.6189453 | 0.9971944 | 0.8055217 |
| AT-3 | 0.8369698 | 1.2876028 | 0.970089 | 1.3910724 | 0.9917418 | 1.0354613 | 1.0201318 |
| Phase-1 RCT-270 | 0.78453 | 0.7997179 | 0.8431378 | 0.9094623 | 0.7613346 | 0.7571247 | 0.6639367 |
| Phase-1 RCT-123 | 1.0540634 | 0.9640542 | 1.0327765 | 0.9379011 | 0.9731166 | 1.0322794 | 1.0002326 |
| Alpha-2-macroglobulin, sequence 2 | 1.1899501 | 1.0210365 | 1.2051609 | 0.9967052 | 0.8969271 | 1.0797632 | 1.3289794 |
| Matrix metalloproteinase-1 | 0.5689108 | 0.82667 | 1.0300617 | 0.9343246 | 0.9084986 | 1.0869126 | 1.0570588 |
| Beta-tubulin, class I | 1.5809557 | 1.2786525 | 0.9403483 | 1.0314034 | 0.9369623 | 0.7972053 | 0.8973138 |
| Phase-1 RCT-161 | 1.4206741 | 0.9565027 | 0.7537702 | 1.2644985 | 1.0436232 | 0.8774453 | 0.7920463 |
| 14-3-3 zeta | 1.3829998 | 1.0083851 | 1.0822741 | 1.1209632 | 0.8370943 | 0.921813 | 0.9276954 |
| Stem cell factor | 0.9511242 | 0.9982795 | 1.1105206 | 1.2063893 | 1.0194987 | 1.0337074 | 0.9428775 |
| Macrophage inflammatory protein-2 alpha | 1.043825 | 1.5169259 | 1.222366 | 1.1931536 | 1.0117675 | 1.1176807 | 1.1524097 |
| Thymidylate synthase | 1.0296001 | 1.1940833 | 1.0217847 | 0.9881159 | 1.2443552 | 1.0538572 | 0.9434331 |
| c-jun | 1.3264308 | 1.0247993 | 1.3437349 | 1.306656 | 1.5004582 | 1.2171007 | 1.3238635 |
| Superoxide dismutase Mn | 1.0536046 | 0.9603038 | 1.0697415 | 1.0276743 | 1.1257976 | 1.128045 | 1.1676245 |
| Phase-1 RCT-73 | 1.2370431 | 0.9856027 | 0.9800001 | 1.0072176 | 0.9117415 | 1.0387224 | 0.9913007 |
| Macrophage inflammatory protein-1 alpha | 1.7815073 | 1.4721205 | 1.1332567 | 1.0996436 | 1.2752298 | 1.2374929 | 1.0984274 |
| Phase-1 RCT-214 | 0.9916065 | 0.8739687 | 0.8811618 | 0.8875946 | 0.7257706 | 0.7823825 | 0.7761771 |
| NADH-cytochrome b5 reductase | 0.7182639 | 0.8766104 | 0.9318636 | 0.8704341 | 0.9718253 | 0.9221873 | 0.851809 |
| Interleukin-1 beta | 0.6190541 | 0.9050888 | 0.9741056 | 0.9875878 | 1.0139403 | 1.0610343 | 1.1265557 |
| NADP-dependent isocitrate | 0.559419 | 0.9058299 | 0.7484161 | 1.1027197 | 0.9581456 | 0.8428694 | 0.8044283 |
| dehydrogenase, cytosolic | | | | | | | |
| Phase-1 RCT-289 | 0.8065083 | 0.8539488 | 1.2053282 | 1.0401973 | 0.9939677 | 1.04399 | 0.9393881 |
| Glutamine synthetase | 0.6593219 | 0.6890682 | 0.9076536 | 0.7599857 | 0.8692796 | 1.1143031 | 1.1458646 |
| Phase-1 RCT-182 | 0.502935 | 1.0811851 | 0.8350681 | 0.8360075 | 0.8512477 | 0.7344849 | 0.7734624 |
| Ubiquitin conjugating enzyme (RAD 6 | 0.7591782 | 1.0174246 | 1.2058126 | 1.1333488 | 1.006145 | 1.1799765 | 1.0975538 |
| homologue) | | | | | | | |
| Extracellular-signal-regulated kinase 1 | 1.6366792 | 0.8984496 | 1.0605581 | 1.2850258 | 1.0756288 | 0.9755187 | 1.0433697 |
| Emerin | 1.0885555 | 0.9879543 | 1.1472399 | 1.0738713 | 1.1182309 | 1.0582236 | 1.0781575 |
| Phase-1 RCT-78 | 0.8610008 | 0.8754257 | 0.9141355 | 1.1193252 | 1.0930681 | 0.9279393 | 0.844425 |
| Phase-1 RCT-212 | 1.1028128 | 1.0394447 | 0.9758118 | 1.0360703 | 1.0193738 | 0.9634308 | 0.9950288 |
| Phase-1 RCT-168 | 0.5888459 | 0.9166398 | 0.8917335 | 0.8687769 | 1.0678431 | 0.7465231 | 0.8092584 |
| Lysyl hydroxylase | 0.6867124 | 1.0399355 | 0.9280525 | 0.8161535 | 1.007509 | 0.983924 | 0.9535422 |
| Phase-1 RCT-55 | 1.0754652 | 1.1051395 | 0.9038169 | 1.257592 | 0.819069 | 0.9695973 | 1.179635 |
| MHC class I antigen RT1.A1(f) alpha- | 2.9960742 | 0.9573503 | 1.4316661 | 1.300423 | 1.3803949 | 1.163116 | 1.3042239 |
| chain | | | | | | | |
| Phase-1 RCT-40 | 0.5044735 | 0.9157996 | 0.7904112 | 1.0646025 | 0.943564 | 0.7239547 | 0.7279773 |
| Cyclin G | 1.4812899 | 1.2407701 | 0.9782873 | 1.0046062 | 1.0156518 | 1.1025336 | 1.2254035 |
| Peroxisome proliferator activated receptor | 1.764595 | 1.3267925 | 1.2063422 | 0.996599 | 1.5850098 | 1.1310933 | 1.0976676 |
| gamma | | | | | | | |
| Phase-1 RCT-280 | 0.8495334 | 0.9606007 | 0.982519 | 0.9892154 | 0.9977027 | 1.1369532 | 1.0490385 |
| D-dopachrome tautomerase | 0.7344272 | 0.8438759 | 0.8000127 | 0.8462267 | 0.8200441 | 0.7043289 | 0.716939 |
| Phase-1 RCT-287 | 0.7629734 | 0.9171231 | 0.9742926 | 1.0620903 | 0.9437181 | 1.1534063 | 1.1490415 |
| Phase-1 RCT-119 | 0.9483053 | 0.9798847 | 1.2690235 | 1.1339738 | 0.940092 | 1.2261946 | 1.2588007 |
| Thioredoxin-1 (Trx1) | 0.6239642 | 1.0301775 | 0.9416493 | 1.1230812 | 1.0713118 | 1.0190364 | 1.008812 |
| Phase-1 RCT-225 | 0.9336601 | 0.8521106 | 1.1730174 | 1.3064153 | 1.6439751 | 1.0069077 | 1.2699995 |
| Protein kinase C alpha | 0.6562914 | 0.970502 | 0.9478711 | 0.9421928 | 1.1282324 | 0.9499368 | 0.9776735 |
| Phase-1 RCT-87 | 0.9052697 | 0.9184353 | 0.9919923 | 1.0109825 | 0.9889612 | 1.0406864 | 1.1337469 |
| Phase-1 RCT-59 | 1.1085469 | 1.1522045 | 1.1858968 | 1.0027959 | 1.0496517 | 1.3092813 | 1.1927016 |
| C4b-binding protein | 1.4171153 | 0.9663627 | 0.8815243 | 1.0091249 | 0.9634552 | 0.8713744 | 0.7801594 |
| Phase-1 RCT-204 | 1.255808 | 0.8708163 | 1.0661837 | 0.9575806 | 0.9822216 | 1.1304886 | 1.1514928 |
| Carnitine palmitoyl-CoA transferase | 1.3395013 | 1.1571155 | 3.821277 | 1.1909064 | 1.1261873 | 1.5373602 | 2.6268623 |
| 8-oxoguanine DNA glycosylase | 1.1986595 | 1.2205598 | 1.0434943 | 0.9880124 | 1.0882015 | 1.1066477 | 1.1859607 |
| H-rev107 | 1.0284055 | 0.9652843 | 0.9886635 | 0.9502911 | 0.9951856 | 1.0533648 | 1.0160178 |
| Preproalbumin | 2.1108387 | 0.7741756 | 1.1888758 | 0.8255016 | 0.6725461 | 0.8583947 | 0.9120059 |
| Phase-1 RCT-177 | 0.8126515 | 0.9196194 | 0.9936718 | 1.0263578 | 0.8708639 | 0.9848983 | 0.899699 |
| Phase-1 RCT-110 | 1.1298898 | 1.1406924 | 1.0010053 | 0.9456122 | 1.0350056 | 1.0281701 | 1.0321643 |
| Phase-1 RCT-227 | 0.6490438 | 0.7737298 | 0.9510952 | 1.1857139 | 0.9551721 | 1.1404179 | 0.8738179 |
| Neuropeptide Y | 1.1380357 | 1.1038357 | 0.957204 | 1.0818069 | 1.0246643 | 1.0265089 | 0.9954924 |
| Phase-1 RCT-277 | 0.6129561 | 1.0278102 | 0.9368885 | 0.7784343 | 0.816762 | 0.9576152 | 0.8339835 |
| Phase-1 RCT-229 | 1.2241085 | 1.1767114 | 0.9211465 | 1.0518948 | 1.0090867 | 1.0167704 | 0.9859735 |
| Phase-1 RCT-34 | 0.7083033 | 0.9653375 | 0.9110316 | 1.227123 | 1.0418711 | 0.8567238 | 0.9186472 |
| Choline kinase | 1.1550108 | 1.3598335 | 0.9688395 | 1.3755882 | 1.2771281 | 1.0615622 | 0.9865583 |
| Phase-1 RCT-20 | 1.3893232 | 1.052828 | 1.0153987 | 1.1305972 | 0.9793928 | 1.0724024 | 1.080595 |
| Phase-1 RCT-248 | 1.0198363 | 0.9768127 | 1.0309949 | 0.9514502 | 1.0965581 | 0.9886219 | 1.0382615 |
| | ISON | KETO | KETO | KETO | KETO | KETO | LPS |
| Compound-Dose (2) | 200 | 20 | 20 | 80 | 80 | 80 | 2 |
| Animal Number (3) | 1953 | 2221 | 2223 | 2231 | 2232 | 2233 | 341 |
| Liver Toxicity Necrosis Classification (4) | no | no | no | no | no | no | no |
| Gene Name (5) | | | | | | | |
| Gadd153 | 1.0536747 | 0.9228966 | 0.9914969 | 1 | 0.9273405 | 0.9907862 | 3.4147527 |
| Interferon related developmental regulator | 0.9246071 | 1.0590816 | 1.095061 | 1.142506 | 1.121716 | 1.0588284 | 1.5046431 |
| IFRD1 (PC4) | | | | | | | |
| ID-1 | 1.0586216 | 1.1144695 | 0.9163131 | 0.9577145 | 0.8889011 | 0.8357365 | 1.1714563 |
| Insulin-like growth factor binding protein 1 | 0.8855669 | 0.9527162 | 0.9959223 | 1.0214511 | 1.0634316 | 0.895165 | 8.229666 |
| NIPK | 1.0726656 | 0.944166 | 0.9670863 | 1.030593 | 1.0444181 | 0.8604865 | 1.0962989 |
| Phase-1 RCT-50 | 1.0860683 | 0.9712822 | 0.9368355 | 1.0838232 | 0.9247001 | 1.0049965 | 2.3360503 |
| Sarcoplasmic reticulum calcium ATPase | 0.9620861 | 0.9715707 | 0.9663368 | 0.9574373 | 0.9752683 | 1.1813173 | 0.9880024 |
| Phase-1 RCT-207 | 1.0593575 | 1.0036439 | 1.0150962 | 0.96733 | 0.9871669 | 0.9589993 | 1.9333489 |
| Acetyl-CoA carboxylase | 0.8410967 | 1.0210835 | 0.9905964 | 1.069679 | 1.0411197 | 1.0051137 | 1.0410357 |
| Cathepsin L, sequence 2 | 1.3108917 | 0.9660311 | 1.2512745 | 0.77504 | 1.0134839 | 1.0899011 | 1.4545306 |
| Glucokinase | 0.4565747 | 0.4627134 | 0.8838492 | 0.7040563 | 0.6418213 | 1.1633391 | 1.2551272 |
| Gadd45 | 0.9120701 | 1.2201904 | 0.8567249 | 1.0077599 | 0.8864299 | 1.277511 | 2.6349094 |
| Phase-1 RCT-18 | 0.952036 | 0.9475948 | 0.9869973 | 0.9782123 | 0.9793334 | 1.0205207 | 0.9364237 |
| 3-beta-hydroxysteroid dehydrogenase | 0.6810293 | 0.968919 | 1.0091366 | 1.0656422 | 0.8923258 | 0.9241464 | 1.0516205 |
| (HSD3B1) | | | | | | | |
| Phase-1 RCT-221 | 1.0511945 | 0.9994119 | 1.0395994 | 1.0425329 | 1.0926871 | 1.0177222 | 1.0888729 |
| Heme oxygenase | 1.3533628 | 1.1688439 | 0.9179193 | 1.4204386 | 1.1380563 | 1.2518272 | 3.533252 |
| Cyclin dependent kinase 4 | 0.9648037 | 1.0089407 | 0.972558 | 0.9971067 | 0.9943976 | 1.0645716 | 0.7521249 |
| Phase-1 RCT-75 | 1.0405363 | 1.0420177 | 0.9328001 | 0.9208476 | 0.9255733 | 0.9381112 | 1.9256188 |
| Phase-1 RCT-65 | 1.1829572 | 1.1986033 | 1.0116844 | 1.0063257 | 1.1034843 | 0.9604521 | 1.8297493 |
| Cytochrome P450 1A1 | 1.5601836 | 0.9850997 | 0.9484124 | 1.0444716 | 0.9862227 | 1.13917 | 0.9304598 |
| Peroxisomal multifunctional enzyme type II | 1.0393927 | 1.0367752 | 1.0424678 | 1.0272564 | 1.0256476 | 0.8668762 | 0.8737555 |
| DNA topoisomerase I | 0.8811902 | 1.1580708 | 0.8709525 | 1.2004129 | 1.1000584 | 1.1760023 | 1.6016743 |
| Focal adhesion kinase (pp125FAK) | 0.8912541 | 0.9701622 | 0.9741653 | 1.0131148 | 0.9664097 | 1.0181396 | 1.671383 |
| NGF-inducible anti-proliferative putative | 0.8653415 | 0.9238848 | 0.937968 | 1.095331 | 1.1392101 | 0.9016523 | 2.760559 |
| secreted protein (PC3) | | | | | | | |
| Phase-1 RCT-82 | 0.9926186 | 0.979524 | 0.9999561 | 0.9841828 | 1.0164995 | 1.0287018 | 0.884632 |
| Senescence marker protein-30 | 0.5446504 | 1.082271 | 1.093525 | 1.1853379 | 1.1663637 | 1.1076714 | 0.2828463 |
| Melanoma-associated antigen ME491 | 1.1305643 | 0.8484442 | 0.8481086 | 1.0443178 | 0.9617333 | 0.9318936 | 1.4767443 |
| Activating transcription factor 3 | 1.0396997 | 1.0964359 | 0.9752201 | 0.9552391 | 0.9464811 | 0.8898185 | 1.7816947 |
| Tryptophan hydroxylase | 0.9875028 | 0.9846308 | 0.874359 | 1.1167656 | 1.004381 | 1.1573745 | 0.7411372 |
| Zinc finger protein | 1.0711578 | 1.0474906 | 1.0601234 | 0.9389355 | 0.9249278 | 0.952601 | 2.6432815 |
| c-myc | 0.9398406 | 1.093317 | 0.9051913 | 1.2135977 | 0.9300202 | 0.9576145 | 2.7813256 |
| Cholesterol 7-alpha-hydroxylase P450 | 0.8521237 | 0.7444301 | 1.256338 | 0.6370072 | 1.2533636 | 1.2613876 | 0.230865 |
| VII) | | | | | | | |
| Ferritin H-chain | 0.841712 | 0.980776 | 0.9569362 | 1.1519245 | 1.0521193 | 1.0327373 | 1.1747218 |
| Phase-1 RCT-197 | 1.3815345 | 1.0674584 | 1.1294522 | 1.1085479 | 1.1012568 | 1.0513813 | 1.63027 |
| Phase-1 RCT-179 | 0.9572802 | 0.9431783 | 1.0555197 | 1.0323646 | 0.9682742 | 0.9215118 | 2.0711462 |
| Phase-1 RCT-15 | 0.9296727 | 1.2411383 | 0.9950888 | 1.363971 | 1.0666254 | 1.1078354 | 1.2746118 |
| Argininosuccinate lyase | 2.6208181 | 1.1160672 | 1.0954962 | 0.8095465 | 0.960001 | 1.0195055 | 0.480956 |
| Phase-1 RCT-49 | 1.0305921 | 0.9557194 | 0.9592778 | 1.1068355 | 1.0002692 | 1.0213331 | 2.304993 |
| Carbonic anhydrase III | 0.3075453 | 1.4889219 | 1.2765006 | 1.2645773 | 0.7353717 | 1.0078796 | 0.3283733 |
| Proliferating cell nuclear antigen gene | 1.0299654 | 0.9249895 | 0.8857636 | 0.9487572 | 0.8741393 | 0.8852767 | 1.6231809 |
| Integrin beta1 | 1.0969945 | 1.1089368 | 1.1159241 | 1.0778109 | 1.1365919 | 0.9384832 | 1.948284 |
| Caspase 6 | 1.0822659 | 1.0233717 | 0.9826703 | 0.9940416 | 1.0026886 | 1.0208048 | 0.7576094 |
| Phase-1 RCT-116 | 1.3454756 | 1.2611448 | 1.0612005 | 0.9222646 | 0.9810332 | 0.9725015 | 0.5983142 |
| Phase-1 RCT-109 | 1.1660628 | 0.9112358 | 0.9469143 | 1.0554012 | 1.0359797 | 0.9956228 | 1.2456201 |
| Phase-1 RCT-71 | 1.2488846 | 0.8846165 | 1.060993 | 0.9642878 | 1.1036463 | 1.1540539 | 1.0625638 |
| Elongation factor-1 alpha | 1.0182295 | 1.0619925 | 1.0313597 | 0.9548139 | 1.0243895 | 0.976929 | 1.330464 |
| Phase-1 RCT-169 | 0.7389251 | 1.0824522 | 1.0230948 | 1.0575747 | 1.0757723 | 1.3243555 | 48.93563 |
| Pyruvate kinase, muscle | 1.1159333 | 0.9935246 | 1.0776857 | 0.9497719 | 1.0369557 | 0.909151 | 0.7058793 |
| Nucleosome assembly protein | 0.9506329 | 1.1925362 | 1.325094 | 0.6179134 | 0.6970356 | 1.0952376 | 0.6243787 |
| Phase-1 RCT-127 | 0.9376865 | 0.9452556 | 0.9944046 | 1.1202545 | 0.9996954 | 1.0909326 | 1.3025404 |
| Iron-responsive element-binding protein | 1.0034567 | 1.0270617 | 1.0318418 | 0.8845413 | 1.2101266 | 0.8743286 | 0.6161139 |
| Phase-1 RCT-72 | 1.1704144 | 0.9644856 | 0.9418323 | 1.0146052 | 0.9565213 | 1.1565528 | 1.2187476 |
| Phase-1 RCT-144 | 0.8513773 | 0.9164333 | 0.9341134 | 1.0557394 | 0.9374691 | 0.9194766 | 1.6013553 |
| Phase-1 RCT-242 | 0.9670332 | 1.0353872 | 0.8869119 | 1.0985578 | 0.9969879 | 1.0035049 | 2.9660199 |
| 17-beta hydroxysteroid dehydrogenase, | 0.9455719 | 1.4876153 | 1.6390032 | 0.4567545 | 0.5456439 | 1.3109382 | 0.2103749 |
| type 2 | | | | | | | |
| Phase-1 RCT-70 | 1.1290449 | 1.0571572 | 1.0744861 | 1.0240337 | 1.1924628 | 0.9736953 | 0.8387432 |
| Ribosomal protein L13A | 1.0647409 | 0.9630259 | 0.9474955 | 1.1012404 | 1.0866926 | 0.9724045 | 1.4947401 |
| Cytochrome P450 2E1 | 1.0906998 | 1.1024042 | 1.0060757 | 0.6459591 | 0.8418387 | 1.0476735 | 0.7931534 |
| AT-3 | 1.0327208 | 0.9828643 | 1.0073847 | 1.0595969 | 0.9622789 | 1.0087639 | 1.0424607 |
| Phase-1 RCT-270 | 0.8706854 | 1.004659 | 0.9690765 | 1.0882479 | 1.0955831 | 1.1575916 | 0.4317776 |
| Phase-1 RCT-123 | 1.0295242 | 0.9570273 | 0.915875 | 0.9966748 | 0.9816834 | 0.9575957 | 0.9983227 |
| Alpha-2-macroglobulin, sequence 2 | 1.2440596 | 0.9831097 | 0.9859294 | 1.0488796 | 0.9737954 | 1.0991638 | 1.2402723 |
| Matrix metalloproteinase-1 | 0.8578755 | 1.0727559 | 0.9830374 | 1.1279216 | 1.0597799 | 1.0368247 | 0.7313246 |
| Beta-tubulin, class I | 0.5525056 | 0.8657698 | 0.6865473 | 1.0923228 | 0.7785361 | 0.7423398 | 1.6665593 |
| Phase-1 RCT-161 | 0.607171 | 1.2304059 | 1.1112796 | 1.1459614 | 0.7651397 | 0.9888889 | 0.8265385 |
| 14-3-3 zeta | 0.8579964 | 0.9799869 | 0.9227863 | 0.9890965 | 0.913325 | 0.8470209 | 1.9878981 |
| Stem cell factor | 0.7633883 | 0.9900981 | 1.069753 | 1.0640366 | 1.0150657 | 0.9979982 | 0.5494676 |
| Macrophage inflammatory protein-2 alpha | 1.1150115 | 1.3293363 | 1.0523502 | 0.9743962 | 0.9353344 | 0.9217366 | 5.360902 |
| Thymidylate synthase | 0.9753314 | 1.283213 | 1.0161449 | 0.9307574 | 0.9592072 | 0.9162862 | 0.7209763 |
| c-jun | 1.3635511 | 1.2254646 | 0.9436435 | 0.8068848 | 0.9074928 | 1.0293031 | 1.4667748 |
| Superoxide dismutase Mn | 1.1636626 | 0.9598176 | 1.0363431 | 1.1149348 | 1.0966605 | 0.9703679 | 20.531837 |
| Phase-1 RCT-73 | 0.9726587 | 0.9718703 | 1.0021045 | 1.0047433 | 0.9802641 | 0.8725392 | 0.8449552 |
| Macrophage inflammatory protein-1 alpha | 1.0442431 | 0.9632984 | 1.0113952 | 1.0514163 | 0.9907138 | 1.0812596 | 9.432194 |
| Phase-1 RCT-214 | 0.61874511 | 1.2086112 | 1.1501125 | 0.9247065 | 1.0162979 | 0.8233785 | 0.444227 |
| NADH-cytochrome b5 reductase | 0.9298016 | 0.7793064 | 0.9567314 | 0.7406374 | 0.9120005 | 0.8200346 | 0.6654027 |
| Interleukin-1 beta | 0.9276232 | 0.9934762 | 0.9376433 | 1.1644472 | 1.0648499 | 1.0146818 | 5.399458 |
| NADP-dependent isocitrate | 0.7353923 | 0.9285606 | 1.1571048 | 0.9219812 | 0.9802655 | 0.9801148 | 0.4421812 |
| dehydrogenase, cytosolic | | | | | | | |
| Phase-1 RCT-289 | 0.9980791 | 0.9380813 | 0.9744469 | 0.9009947 | 0.9569861 | 0.9224083 | 0.7643685 |
| Glutamine synthetase | 1.2060397 | 0.8932053 | 0.9068219 | 0.9232394 | 0.8141592 | 1.0149715 | 2.564585 |
| Phase-1 RCT-182 | 0.9121343 | 0.890106 | 0.9173531 | 0.8357683 | 0.8645767 | 0.9069827 | 0.7525027 |
| Ubiquitin conjugating enzyme (RAD 6 | 1.0041174 | 0.9653044 | 1.0942245 | 0.951561 | 0.9729593 | 0.8963175 | 2.1065512 |
| homologue) | | | | | | | |
| Extracellular-signal-regulated kinase 1 | 0.7869572 | 1.3799845 | 1.2503301 | 1.2218295 | 0.8259686 | 1.0389146 | 0.6245113 |
| Emerin | 1.03155 | 0.9834827 | 1.0285655 | 1.0232941 | 1.0402478 | 0.8535997 | 0.8224931 |
| Phase-1 RCT-78 | 0.9554932 | 1.0396174 | 1.1255184 | 0.9098462 | 0.9854085 | 0.9995112 | 0.9443744 |
| Phase-1 RCT-212 | 0.9932917 | 0.8835548 | 0.9999468 | 1.0203595 | 1.0179217 | 1.0982866 | 0.8302221 |
| Phase-1 RCT-168 | 0.7108972 | 0.9605346 | 1.0633084 | 0.8640158 | 0.9290697 | 0.901916 | 0.6164324 |
| Lysyl hydroxylase | 0.9791673 | 0.9072464 | 0.9281597 | 0.9817754 | 0.9135438 | 0.9275596 | 1.3596615 |
| Phase-1 RCT-55 | 0.9249211 | 0.835499 | 0.8632738 | 0.9511315 | 0.9088479 | 0.954855 | 1.1280015 |
| MHC class I antigen RT1.A1(f) alpha- | 1.2738571 | 1.0519633 | 1.0611436 | 0.9614006 | 1.1066346 | 0.838361 | 1.5966864 |
| chain | | | | | | | |
| Phase-1 RCT-40 | 0.7241561 | 0.97362 | 0.8867274 | 1.020949 | 0.9851807 | 0.7941262 | 0.6146483 |
| Cyclin G | 1.1592332 | 1.0911762 | 1.1682767 | 1.1554235 | 1.0402974 | 1.0339515 | 3.248979 |
| Peroxisome proliferator activated receptor | 1.1105576 | 1.3675216 | 1.0355848 | 0.9186547 | 0.936335 | 0.8823621 | 0.6611542 |
| gamma | | | | | | | |
| Phase-1 RCT-280 | 1.1940002 | 1.2342781 | 1.0894498 | 1.2267095 | 1.0038025 | 0.8396637 | 0.9345424 |
| D-dopachrome tautomerase | 0.7748854 | 0.7865582 | 0.8992852 | 0.8667085 | 0.9371771 | 0.9652985 | 0.6214878 |
| Phase-1 RCT-287 | 1.1162273 | 0.9477836 | 0.9604146 | 1.0962455 | 0.9772052 | 0.8461351 | 0.9582894 |
| Phase-1 RCT-119 | 1.2930411 | 1.0418226 | 0.9681125 | 0.8699712 | 0.9049466 | 1.2729532 | 0.6594039 |
| Thioredoxin-1 (Trx1) | 0.8663642 | 0.9080933 | 0.9667903 | 1.100456 | 1.1160744 | 0.9965038 | 2.822328 |
| Phase-1 RCT-225 | 2.112223 | 0.8094559 | 1.1413121 | 1.0842501 | 1.0993338 | 0.7192935 | 1.0377507 |
| Protein kinase C alpha | 1.0001739 | 0.9302001 | 0.9086485 | 0.978628 | 0.9818844 | 0.9488993 | 0.8701847 |
| Phase-1 RCT-87 | 1.1169786 | 0.913342 | 1.0042148 | 1.0193317 | 1.0516809 | 1.02899 | 1.4517434 |
| Phase-1 RCT-59 | 0.9986311 | 1.0225185 | 0.9976539 | 1.0598515 | 1.0568336 | 1.0814726 | 2.68238 |
| C4b-binding protein | 0.6744671 | 1.3965802 | 1.073919 | 1.36641 | 1.0807018 | 1.303682 | 0.8989053 |
| Phase-1 RCT-204 | 1.1952876 | 0.9873692 | 0.9944999 | 0.9995009 | 0.9722573 | 1.0208148 | 0.9971521 |
| Carnitine palmitoyl-CoA transferase | 2.2839532 | 1.3156136 | 1.5311923 | 0.7871196 | 0.8836854 | 0.8662098 | 0.5530677 |
| 8-oxoguanine DNA glycosylase | 1.0870602 | 1.0826445 | 1.0254344 | 1.1597703 | 1.0345509 | 1.071013 | 0.8076708 |
| H-rev107 | 1.0201252 | 1.1654503 | 1.0211188 | 1.0884885 | 1.0170335 | 1.0368363 | 1.1952735 |
| Preproalbumin | 0.7670341 | 1.1105329 | 1.0150822 | 1.101477 | 0.9570871 | 0.9398915 | 0.9298036 |
| Phase-1 RCT-177 | 0.9107521 | 1.0154182 | 1.028458 | 1.0590694 | 1.0333862 | 0.9006326 | 0.7138494 |
| Phase-1 RCT-110 | 1.0382303 | 0.9763002 | 0.9388783 | 0.9899639 | 1.0177981 | 1.1037645 | 0.977665 |
| Phase-1 RCT-227 | 1.1657801 | 1.2050165 | 1.5869582 | 0.8670942 | 0.9882239 | 0.9907143 | 0.7694052 |
| Neuropeptide Y | 1.0244286 | 0.9760039 | 0.9869478 | 1.0055175 | 0.9759518 | 1.0927428 | 1.388911 |
| Phase-1 RCT-277 | 0.8128217 | 0.9076815 | 0.940818 | 0.9199227 | 1.4039158 | 1.0100731 | 1.0874782 |
| Phase-1 RCT-229 | 1.0636351 | 0.9705339 | 1.0186312 | 0.9422853 | 1.0007714 | 1.0198772 | 0.9173963 |
| Phase-1 RCT-34 | 0.6565795 | 1.0098627 | 0.8561175 | 0.9200938 | 0.9737912 | 0.8848324 | 0.4185021 |
| Choline kinase | 1.0498606 | 0.9060388 | 0.8861996 | 0.9791244 | 0.9503803 | 1.0580543 | 0.9651046 |
| Phase-1 RCT-20 | 1.0999168 | 0.9448681 | 0.9703032 | 0.9917864 | 0.9711199 | 1.04403 | 0.9273385 |
| Phase-1 RCT-248 | 1.0740494 | 0.9741902 | 0.9360652 | 1.0378385 | 0.9905203 | 1.0141878 | 0.9772486 |
| | LPS | LPS | LPS | LPS | LPS | MET | MET |
| Compound-Dose (2) | 2 | 2 | 8 | 8 | 8 | 1.3 | 1.3 |
| Animal Number (3) | 342 | 343 | 351 | 352 | 353 | 221 | 222 |
| Liver Toxicity Necrosis Classification (4) | no | no | yes | yes | yes | no | no |
| Gene Name (5) | | | | | | | |
| Gadd153 | 1.6607026 | 3.3599277 | 2.1310408 | 4.285916 | 2.851768 | 1.6387256 | 1.7123194 |
| Interferon related developmental regulator | 1.1177357 | 1.2243353 | 1.842565 | 1.6230091 | 1.4814252 | 0.6656938 | 0.6916686 |
| IFRD1 (PC4) | | | | | | | |
| ID-1 | 0.8479681 | 1.0369929 | 1.0392978 | 1.0273883 | 1.2712274 | 2.058135 | 1.7220719 |
| Insulin-like growth factor binding protein 1 | 3.770373 | 3.7279167 | 2.0381908 | 18.500338 | 4.588317 | 0.7659578 | 0.836329 |
| NIPK | 1.0707154 | 1.143029 | 0.9314405 | 1.132536 | 1.1398163 | 1.6592982 | 1.7507889 |
| Phase-1 RCT-50 | 2.4759953 | 3.35019 | 1.4986522 | 3.6602044 | 2.5695453 | 1.3871026 | 1.3971759 |
| Sarcoplasmic reticulum calcium ATPase | 1.065433 | 0.9481217 | 0.8891953 | 1.0419314 | 0.8204625 | 0.5076294 | 0.4089853 |
| Phase-1 RCT-207 | 1.8592138 | 1.1162423 | 1.2659117 | 1.5635973 | 0.7206125 | 1.1188117 | 1.065494 |
| Acetyl-CoA carboxylase | 0.7440718 | 0.9877024 | 0.8138889 | 0.5847421 | 0.7213042 | 0.9144555 | 1.0344888 |
| Cathepsin L, sequence 2 | 0.9989637 | 1.1937855 | 1.3231181 | 2.5843117 | 2.0065484 | 0.8073406 | 1.0012062 |
| Glucokinase | 0.7959354 | 0.9615579 | 0.8283927 | 0.4372022 | 0.6141817 | 1.094789 | 1.2558228 |
| Gadd45 | 2.1587982 | 2.3142595 | 2.8278213 | 2.7900846 | 3.5787094 | 0.9727326 | 1.0368172 |
| Phase-1 RCT-18 | 0.9858875 | 0.9827222 | 0.8962061 | 0.6917099 | 0.8668181 | 1.0475577 | 0.9659801 |
| 3-beta-hydroxysteroid dehydrogenase | 1.8228394 | 0.8662202 | 1.1989218 | 0.8422013 | 0.5904704 | 0.9768385 | 0.8232995 |
| (HSD3B1) | | | | | | | |
| Phase-1 RCT-221 | 1.1444511 | 0.9079188 | 0.85457 | 0.8301904 | 0.9822098 | 0.9634847 | 0.866753 |
| Heme oxygenase | 3.4081213 | 4.7782593 | 1.6366897 | 11.614569 | 4.120745 | 0.8398098 | 0.917912 |
| Cyclin dependent kinase 4 | 0.9116862 | 0.842817 | 1.0302713 | 0.9549416 | 0.8847669 | 1.470408 | 1.2557586 |
| Phase-1 RCT-75 | 1.679687 | 1.4774226 | 1.4459257 | 1.929228 | 1.7049214 | 0.9844463 | 0.9640312 |
| Phase-1 RCT-65 | 2.3381355 | 2.0400748 | 1.1506512 | 1.5752776 | 1.6146058 | 2.1649675 | 2.3416471 |
| Cytochrome P450 1A1 | 1.0024225 | 1.3778642 | 0.6076481 | 0.4040059 | 0.4211884 | 2.2584696 | 1.9025488 |
| Peroxisomal multifunctional enzyme type II | 0.4858698 | 0.5794489 | 0.8519087 | 0.7063619 | 0.7191429 | 1.037631 | 0.9540434 |
| DNA topoisomerase I | 1.4623646 | 1.2654377 | 1.4454291 | 1.4159832 | 1.6161628 | 0.4169716 | 0.5620475 |
| Focal adhesion kinase (pp125FAK) | 1.3634831 | 1.519662 | 1.5010269 | 1.4551363 | 1.7232593 | 1.0319128 | 1.0320194 |
| NGF-inducible anti-proliferative putative | 1.8117344 | 2.8377779 | 2.0227947 | 3.49445 | 2.9348636 | 0.6492041 | 0.7670667 |
| secreted protein (PC3) | | | | | | | |
| Phase-1 RCT-82 | 0.7904565 | 1.014286 | 0.8498794 | 0.765677 | 0.9055275 | 1.0177155 | 0.8753635 |
| Senescence marker protein-30 | 0.3149866 | 0.3388356 | 0.2672288 | 0.2353374 | 0.2370538 | 0.3853445 | 0.4271487 |
| Melanoma-associated antigen ME491 | 1.4097823 | 1.6094047 | 1.3896492 | 1.6730782 | 2.1062632 | 0.9613594 | 0.9777685 |
| Activating transcription factor 3 | 1.7727222 | 1.231608 | 0.9486762 | 1.5747051 | 0.7431607 | 1.7054068 | 2.2322686 |
| Tryptophan hydroxylase | 0.995548 | 0.7857753 | 0.7581213 | 0.5778653 | 0.7021139 | 1.0095191 | 1.3256533 |
| Zinc finger protein | 2.9504304 | 1.4631441 | 1.3831595 | 5.941812 | 2.420045 | 1.0439065 | 1.1287794 |
| c-myc | 1.5247602 | 1.8029577 | 1.2743174 | 3.7306855 | 1.998253 | 1.7474566 | 1.6644868 |
| Cholesterol 7-alpha-hydroxylase P450 | 0.6767724 | 0.4512604 | 0.7565269 | 0.8305054 | 0.785973 | 1.2541755 | 1.0806594 |
| VII) | | | | | | | |
| Ferritin H-chain | 0.7769795 | 1.0179391 | 1.0713936 | 1.3411149 | 1.4399604 | 0.6834927 | 0.6263039 |
| Phase-1 RCT-197 | 1.8056266 | 1.380795 | 1.3244987 | 1.1843028 | 1.2471777 | 0.9695849 | 1.103641 |
| Phase-1 RCT-179 | 1.7609483 | 1.1678132 | 2.3016577 | 1.9836354 | 1.7305132 | 0.7943228 | 0.7373452 |
| Phase-1 RCT-15 | 1.0940305 | 1.3604777 | 1.3757702 | 1.7532504 | 1.6626421 | 1.6727176 | 2.1789854 |
| Argininosuccinate lyase | 0.8458837 | 0.5143747 | 1.0603545 | 1.6386608 | 1.323768 | 1.0113677 | 1.0245849 |
| Phase-1 RCT-49 | 2.8278906 | 2.124291 | 1.409815 | 2.2399306 | 2.2527413 | 0.9473326 | 0.9340438 |
| Carbonic anhydrase III | 0.1367678 | 1.0052402 | 0.1754519 | 0.1191592 | 0.1100009 | 0.6417587 | 0.7571603 |
| Proliferating cell nuclear antigen gene | 1.254984 | 1.3308322 | 1.0204445 | 2.056137 | 1.4535215 | 1.4740138 | 1.3691646 |
| Integrin beta1 | 2.2543967 | 1.691816 | 1.3210272 | 3.7003593 | 1.8610729 | 1.0598472 | 1.0449225 |
| Caspase 6 | 1.0710107 | 0.790594 | 0.8747137 | 1.2563053 | 0.9847196 | 1.3844489 | 1.3780744 |
| Phase-1 RCT-116 | 1.0733088 | 0.733268 | 0.9838448 | 1.3415624 | 1.2693154 | 2.9811418 | 2.8088133 |
| Phase-1 RCT-109 | 1.6510758 | 1.0729297 | 1.1496828 | 1.2894638 | 1.2747709 | 0.4924123 | 0.5262826 |
| Phase-1 RCT-71 | 1.4720643 | 1.0881327 | 1.035437 | 2.033831 | 1.3607856 | 0.9468104 | 0.9262803 |
| Elongation factor-1 alpha | 1.1346995 | 1.126819 | 1.2626036 | 1.4459492 | 1.3983423 | 0.7502229 | 0.8597646 |
| Phase-1 RCT-169 | 85.23488 | 36.190662 | 28.334414 | 50.985996 | 48.983387 | 1.0867108 | 0.842231 |
| Pyruvate kinase, muscle | 1.0265826 | 1.1384441 | 1.2984685 | 2.5647087 | 1.8940941 | 1.056601 | 1.2424566 |
| Nucleosome assembly protein | 0.5608808 | 0.8003915 | 0.6643791 | 0.5956513 | 0.6796462 | 0.8322721 | 0.8848684 |
| Phase-1 RCT-127 | 1.2364187 | 1.5875487 | 1.2943637 | 2.0298467 | 1.6490371 | 0.9575567 | 0.9470899 |
| Iron-responsive element-binding protein | 0.4021433 | 0.5363564 | 0.7518585 | 0.4675145 | 0.5653893 | 1.0240958 | 0.9408677 |
| Phase-1 RCT-72 | 1.369233 | 1.1061096 | 1.1816725 | 1.4818263 | 1.5678983 | 0.9259833 | 1.000445 |
| Phase-1 RCT-144 | 1.3763506 | 1.4749446 | 1.1437457 | 1.6977193 | 1.3412372 | 1.0115086 | 0.8179072 |
| Phase-1 RCT-242 | 4.75165 | 3.497128 | 1.7929516 | 2.57932 | 3.6428614 | 1.2112051 | 1.1058719 |
| 17-beta hydroxysteroid dehydrogenase, | 0.3442984 | 0.5062181 | 0.2978527 | 0.223705 | 0.2191122 | 0.7175208 | 0.835137 |
| type 2 | | | | | | | |
| Phase-1 RCT-70 | 0.7820103 | 0.8275557 | 0.6928653 | 0.5595976 | 0.6027919 | 1.1632639 | 0.9957719 |
| Ribosomal protein L13A | 1.6624883 | 1.2439847 | 1.2858732 | 1.5497344 | 1.5530978 | 1.0576942 | 1.0758667 |
| Cytochrome P450 2E1 | 0.4247553 | 0.78982 | 0.6889061 | 0.5190199 | 0.6842659 | 1.6259478 | 1.2908205 |
| AT-3 | 0.9683421 | 1.0546159 | 1.1529136 | 1.1333535 | 1.2718793 | 0.9432427 | 0.9593796 |
| Phase-1 RCT-270 | 0.4603207 | 0.4836426 | 0.5108092 | 0.2469673 | 0.3804783 | 0.5518974 | 0.63035 |
| Phase-1 RCT-123 | 0.8135947 | 1.0918111 | 0.9628128 | 1.1209729 | 1.0576805 | 1.1343591 | 1.0553668 |
| Alpha-2-macroglobulin, sequence 2 | 1.5531377 | 1.2850043 | 1.3945466 | 1.7211853 | 1.429218 | 0.8741447 | 0.9336213 |
| Matrix metalloproteinase-1 | 0.9457606 | 0.8353539 | 0.9790733 | 1.2289183 | 1.3289477 | 0.774088 | 0.7834659 |
| Beta-tubulin, class I | 1.3413569 | 1.7304004 | 1.1878859 | 1.3599939 | 1.3606906 | 1.5405242 | 1.2587079 |
| Phase-1 RCT-161 | 0.3891223 | 0.9803423 | 0.9279446 | 0.7865819 | 0.9114956 | 2.3014426 | 1.9659631 |
| 14-3-3 zeta | 1.9108706 | 1.7060933 | 1.3774607 | 2.404773 | 1.5407195 | 1.3666638 | 1.3138492 |
| Stem cell factor | 0.7966409 | 0.8066062 | 0.4735835 | 0.3467059 | 0.3413201 | 1.3101155 | 1.5282516 |
| Macrophage inflammatory protein-2 alpha | 4.1258607 | 2.837201 | 1.4550626 | 2.597739 | 2.5921078 | 1.0994107 | 1.1934859 |
| Thymidylate synthase | 0.9231061 | 1.0498892 | 0.7390853 | 0.5231131 | 0.4614306 | 1.2370056 | 1.3519739 |
| c-jun | 1.4625305 | 1.8214475 | 0.8356571 | 1.0038534 | 1.250455 | 1.4467466 | 1.5606412 |
| Superoxide dismutase Mn | 12.397959 | 16.049902 | 13.238676 | 25.926502 | 21.8753 | 0.9747338 | 1.0315026 |
| Phase-1 RCT-73 | 0.6589457 | 0.8202743 | 1.0047376 | 0.812212 | 0.5999307 | 1.2580303 | 1.1285874 |
| Macrophage inflammatory protein-1 alpha | 2.2058752 | 1.5598359 | 1.9972274 | 9.395047 | 2.5032089 | 1.9005556 | 2.2255218 |
| Phase-1 RCT-214 | 0.2547292 | 0.4908926 | 0.8169841 | 0.915823 | 0.5482919 | 1.1927806 | 1.0708815 |
| NADH-cytochrome b5 reductase | 0.367132 | 0.6054735 | 0.535887 | 0.3365015 | 0.4537689 | 0.7588252 | 0.711055 |
| Interleukin-1 beta | 1.2263435 | 2.880983 | 1.1058053 | 1.424196 | 1.5072981 | 0.8950957 | 0.8709363 |
| NADP-dependent isocitrate | 0.2603418 | 0.3997504 | 0.674601 | 0.3465505 | 0.3917808 | 0.7229914 | 0.6312189 |
| dehydrogenase, cytosolic | | | | | | | |
| Phase-1 RCT-289 | 0.5165652 | 0.7193102 | 0.6661751 | 0.5658197 | 0.6869847 | 0.7683361 | 0.791154 |
| Glutamine synthetase | 1.6971004 | 1.9901379 | 1.2630124 | 1.17765 | 1.4402163 | 0.8578215 | 0.8370555 |
| Phase-1 RCT-182 | 0.489632 | 0.7202601 | 0.4762563 | 0.4048491 | 0.5181661 | 0.6825464 | 0.6786026 |
| Ubiquitin conjugating enzyme (RAD 6 | 1.6101443 | 1.3191372 | 1.5347537 | 2.276446 | 1.857328 | 0.9810194 | 0.8886622 |
| homologue) | | | | | | | |
| Extracellular-signal-regulated kinase 1 | 0.9403281 | 1.0643568 | 0.2183505 | 0.1415699 | 0.1394627 | 1.4962692 | 1.7380564 |
| Emerin | 0.8678658 | 0.9476531 | 0.8109267 | 0.7483903 | 0.5317437 | 1.0777112 | 1.1288517 |
| Phase-1 RCT-78 | 0.7068965 | 1.0357363 | 0.7241861 | 0.7169935 | 0.7649793 | 0.9068503 | 1.0068777 |
| Phase-1 RCT-212 | 0.7993874 | 0.8872975 | 1.0228524 | 1.011959 | 1.0400214 | 1.2010728 | 1.073962 |
| Phase-1 RCT-168 | 0.3835167 | 0.6625264 | 0.7827294 | 0.5803307 | 0.5531787 | 0.611268 | 0.6242065 |
| Lysyl hydroxylase | 1.4077295 | 1.268237 | 1.0859128 | 1.0776505 | 1.1825135 | 0.8742588 | 0.8487374 |
| Phase-1 RCT-55 | 1.2206091 | 1.0474926 | 1.12711 | 1.5746086 | 1.5776507 | 0.6394867 | 0.5346103 |
| MHC class I antigen RT1.A1(f) alpha- | 1.6918482 | 1.9732311 | 1.5030735 | 2.1590161 | 2.450555 | 5.509003 | 4.330819 |
| chain | | | | | | | |
| Phase-1 RCT-40 | 0.4282304 | 0.5168296 | 0.612667 | 0.4714623 | 0.4964311 | 0.5582312 | 0.6684598 |
| Cyclin G | 3.4577875 | 2.9949992 | 1.5611303 | 3.1243408 | 2.611184 | 1.4523977 | 1.660195 |
| Peroxisome proliferator activated receptor | 0.8398752 | 0.9596175 | 0.5613834 | 0.4179068 | 0.3650779 | 1.6823552 | 2.0147614 |
| gamma | | | | | | | |
| Phase-1 RCT-280 | 0.7705086 | 0.8217155 | 1.0696307 | 1.0215472 | 0.9014245 | 0.9354727 | 1.0299921 |
| D-dopachrome tautomerase | 0.3593805 | 0.4566949 | 0.4838556 | 0.4243018 | 0.4995816 | 0.6687587 | 0.6962988 |
| Phase-1 RCT-287 | 0.4609412 | 0.7396115 | 0.9858619 | 0.7802296 | 0.7561602 | 0.9893184 | 0.9464392 |
| Phase-1 RCT-119 | 0.6327754 | 0.7968408 | 0.5696921 | 0.4240459 | 0.5633097 | 0.6634553 | 0.7265123 |
| Thioredoxin-1 (Trx1) | 2.4958155 | 2.0429754 | 2.3559551 | 2.6454837 | 2.7709494 | 0.4623632 | 0.5159885 |
| Phase-1 RCT-225 | 1.0879977 | 0.8283416 | 1.0797901 | 1.2393788 | 1.6334157 | 1.6080526 | 1.4475479 |
| Protein kinase C alpha | 1.008037 | 0.7707009 | 0.8777539 | 0.9323966 | 0.8934742 | 0.7604998 | 0.7101405 |
| Phase-1 RCT-87 | 1.4073529 | 1.1859186 | 1.3436353 | 1.074945 | 1.5530536 | 0.7801647 | 0.8196669 |
| Phase-1 RCT-59 | 2.1432896 | 1.2829205 | 1.3543965 | 2.5287957 | 0.8281634 | 0.7822855 | 1.0337437 |
| C4b-binding protein | 0.9883078 | 1.0573431 | 1.9719384 | 2.217614 | 2.1400852 | 1.1352882 | 0.8873498 |
| Phase-1 RCT-204 | 0.9422084 | 1.1793935 | 1.09233 | 1.2205852 | 1.3370055 | 1.1363025 | 1.1313006 |
| Carnitine palmitoyl-CoA transferase | 1.6048687 | 0.7703967 | 0.8100183 | 0.8295691 | 0.4676666 | 1.2391233 | 1.5312753 |
| 8-oxoguanine DNA glycosylase | 1.0542516 | 1.2042111 | 0.9814477 | 0.8092792 | 0.9434693 | 1.1086553 | 1.1691469 |
| H-rev107 | 1.1315634 | 1.1548426 | 1.1323792 | 1.3529751 | 1.8248473 | 1.1732174 | 1.0663573 |
| Preproalbumin | 0.5736989 | 0.9290387 | 1.3461627 | 1.2906474 | 1.332073 | 1.7008556 | 1.5603569 |
| Phase-1 RCT-177 | 0.7494623 | 0.5520546 | 0.9722184 | 0.8812247 | 0.7464364 | 0.8374366 | 0.8639364 |
| Phase-1 RCT-110 | 1.0942925 | 1.2048389 | 0.9443737 | 1.2539673 | 1.1334356 | 1.2568412 | 1.2213258 |
| Phase-1 RCT-227 | 0.5418113 | 1.0283946 | 0.7056301 | 0.5750716 | 0.6493662 | 0.7290695 | 0.8075896 |
| Neuropeptide Y | 0.9017201 | 1.3144174 | 0.9399234 | 1.3766555 | 1.1883028 | 1.1026388 | 1.0677933 |
| Phase-1 RCT-277 | 0.527112 | 0.8771951 | 1.1785953 | 1.2288909 | 1.1694478 | 0.8633753 | 0.8046255 |
| Phase-1 RCT-229 | 0.8978174 | 1.0965973 | 0.8273019 | 0.8226742 | 0.8515185 | 1.3243006 | 1.2748146 |
| Phase-1 RCT-34 | 0.4070748 | 0.5891155 | 0.8146992 | 0.7392666 | 0.7838734 | 0.7148867 | 0.6688721 |
| Choline kinase | 1.2172351 | 1.1012555 | 0.942351 | 1.1766359 | 1.0678389 | 1.3631154 | 1.7612989 |
| Phase-1 RCT-20 | 0.994029 | 1.0900743 | 0.9288963 | 0.99154 | 0.9225699 | 1.3838162 | 1.543528 |
| Phase-1 RCT-248 | 1.2599342 | 1.0936613 | 1.056177 | 1.0893133 | 1.0882382 | 0.9072105 | 0.7830525 |
| | MET | MET | MET | NAL | NAL | NAL | NAL |
| Compound-Dose (2) | 1.3 | 5 | 5 | 45 | 45 | 45 | 180 |
| Animal Number (3) | 223 | 232 | 233 | 2641 | 2642 | 2643 | 2651 |
| Liver Toxicity Necrosis Classification (4) | no | no | no | no | no | no | no |
| Gene Name (5) | | | | | | | |
| Gadd153 | 1.2044247 | 1.5656763 | 1.4602768 | 0.7791075 | 0.8370411 | 1.8379778 | 0.9951789 |
| Interferon related developmental regulator | 0.6556312 | 0.7996874 | 0.7825933 | 1.1047945 | 0.9872357 | 1.2663137 | 1.0433071 |
| IFRD1 (PC4) | | | | | | | |
| ID-1 | 1.6571474 | 1.2788585 | 1.5070465 | 1.1378957 | 1.1266406 | 2.565162 | 1.0404894 |
| Insulin-like growth factor binding protein 1 | 0.6807259 | 0.4722701 | 0.6238672 | 0.9155845 | 0.9703859 | 1.0815454 | 1.4179277 |
| NIPK | 1.5361422 | 1.1310045 | 1.0576954 | 1.0257678 | 1.2061241 | 1.3690506 | 1.1256688 |
| Phase-1 RCT-50 | 1.4273196 | 1.4594349 | 1.3769141 | 0.891236 | 0.8853963 | 0.9207312 | 0.9718036 |
| Sarcoplasmic reticulum calcium ATPase | 0.4373581 | 0.7981359 | 0.706964 | 0.9798018 | 0.8154047 | 1.1012964 | 0.9825248 |
| Phase-1 RCT-207 | 1.2976832 | 1.1162758 | 0.7513261 | 1.1035242 | 1.1265339 | 1.0290045 | 1.121846 |
| Acetyl-CoA carboxylase | 0.6853623 | 1.0089206 | 1.0234956 | 0.9612424 | 0.8863754 | 0.8575997 | 0.9250437 |
| Cathepsin L, sequence 2 | 0.8251112 | 1.329302 | 1.2228293 | 1.0222241 | 1.0717591 | 1.1257784 | 1.0588249 |
| Glucokinase | 0.5736947 | 1.0199665 | 1.115394 | 0.8868384 | 1.2858275 | 1.0520258 | 0.4916571 |
| Gadd45 | 0.9390754 | 0.7329065 | 0.9197177 | 0.9423077 | 1.137563 | 0.8683834 | 1.0540577 |
| Phase-1 RCT-18 | 1.2029899 | 0.9911901 | 1.0924424 | 0.9344946 | 0.870634 | 0.8300904 | 0.8838441 |
| 3-beta-hydroxysteroid dehydrogenase | 1.0041775 | 0.7312996 | 0.6601865 | 1.2633917 | 1.3496358 | 0.9574679 | 0.9385622 |
| (HSD3B1) | | | | | | | |
| Phase-1 RCT-221 | 0.9791629 | 0.7351127 | 1.266469 | 1.149695 | 1.1092149 | 1.2413803 | 1.1519644 |
| Heme oxygenase | 0.8519703 | 0.9668901 | 1.129992 | 1.9946654 | 1.9984113 | 2.3341227 | 2.8951182 |
| Cyclin dependent kinase 4 | 1.1908913 | 1.6716791 | 1.6995487 | 0.8564991 | 1.1177077 | 1.1014198 | 1.1043694 |
| Phase-1 RCT-75 | 1.0278354 | 1.1472554 | 1.1335636 | 1.2784487 | 1.2602512 | 1.1567805 | 1.083776 |
| Phase-1 RCT-65 | 1.917057 | 1.3282099 | 2.3091102 | 0.9800724 | 0.9121468 | 0.8185287 | 1.0352284 |
| Cytochrome P450 1A1 | 2.4119864 | 1.7449286 | 1.4757196 | 0.7732766 | 0.8122972 | 0.9242477 | 0.9132097 |
| Peroxisomal multifunctional enzyme type II | 1.3137789 | 1.2228757 | 1.4286499 | 1.3264323 | 1.0669152 | 1.2024528 | 1.1055452 |
| DNA topoisomerase I | 0.6377395 | 0.7508929 | 0.6740785 | 1.0025634 | 1.0271807 | 1.086932 | 0.8035574 |
| Focal adhesion kinase (pp125FAK) | 0.9834468 | 0.9562978 | 1.004082 | 0.844397 | 0.9757326 | 1.0451201 | 0.983977 |
| NGF-inducible anti-proliferative putative | 0.8268963 | 0.4787998 | 0.7276106 | 1.0113196 | 0.8238075 | 0.8602029 | 1.0579156 |
| secreted protein (PC3) | | | | | | | |
| Phase-1 RCT-82 | 0.9152693 | 0.8834263 | 1 | 0.9266159 | 0.847594 | 0.8225809 | 0.913681 |
| Senescence marker protein-30 | 0.5167382 | 0.9871358 | 0.7702801 | 1.1746048 | 1.05583 | 1.0259887 | 0.7912152 |
| Melanoma-associated antigen ME491 | 1.0079892 | 0.9737937 | 1.1212779 | 1.0566019 | 1.0035516 | 1.5497603 | 1.1232235 |
| Activating transcription factor 3 | 2.350112 | 1.5404048 | 1.2147006 | 1.2879127 | 1.5620657 | 1.4774483 | 1.3906368 |
| Tryptophan hydroxylase | 1.3316151 | 1.1581575 | 0.9747872 | 0.9714493 | 1.0400838 | 0.89465 | 0.9917306 |
| Zinc finger protein | 1.34541 | 1.0655411 | 0.8659261 | 0.9122602 | 1.0465122 | 1.0982033 | 1.0268971 |
| c-myc | 1.6195909 | 1.6347424 | 1.6002514 | 0.9212689 | 1.2761987 | 1.0178604 | 1.119941 |
| Cholesterol 7-alpha-hydroxylase P450 | 0.8333877 | 1.3587931 | 1.3904295 | 0.5793515 | 0.9409087 | 0.7491255 | 0.9356187 |
| VII) | | | | | | | |
| Ferritin H-chain | 0.5233888 | 0.5706344 | 0.7916933 | 0.965289 | 0.9285261 | 0.9881395 | 1.7217417 |
| Phase-1 RCT-197 | 0.9901083 | 0.8860568 | 1.0120423 | 0.9326422 | 0.9007457 | 1.0202954 | 0.986041 |
| Phase-1 RCT-179 | 0.860205 | 0.6904762 | 0.6119167 | 1.2181811 | 1.2942263 | 1.3986197 | 0.9849422 |
| Phase-1 RCT-15 | 1.4221032 | 2.5069609 | 1.5998527 | 1.240706 | 1.110488 | 0.8757532 | 1.0327007 |
| Argininosuccinate lyase | 1.3214672 | 1.319278 | 1.4901671 | 0.998091 | 1.2113259 | 1.4027214 | 1.3859622 |
| Phase-1 RCT-49 | 1.0148873 | 0.8084245 | 0.9657586 | 0.9850292 | 1.0414908 | 1.0148319 | 1.2372597 |
| Carbonic anhydrase III | 0.5190741 | 1.0308634 | 0.5695257 | 0.899995 | 0.7736411 | 0.6952053 | 0.4929818 |
| Proliferating cell nuclear antigen gene | 0.9504591 | 1.341481 | 0.9950362 | 1.0171561 | 1.0910658 | 1.0994637 | 1.1698159 |
| Integrin beta1 | 1.0848004 | 0.937654 | 1.0565115 | 1.0856451 | 0.9953101 | 1.0281152 | 1.3164912 |
| Caspase 6 | 1.064221 | 1.1083107 | 1.0865866 | 0.8604817 | 0.9374215 | 1.1174734 | 1.1628164 |
| Phase-1 RCT-116 | 4.3035154 | 3.3481505 | 3.759344 | 0.9367794 | 0.9549914 | 0.8752465 | 1.2648683 |
| Phase-1 RCT-109 | 0.758065 | 0.3605688 | 0.5422646 | 1.0476534 | 0.9840335 | 1.1953714 | 1.2668154 |
| Phase-1 RCT-71 | 1.0715241 | 1.0643274 | 1.0670989 | 0.8677384 | 0.8124172 | 0.8103629 | 0.9418117 |
| Elongation factor-1 alpha | 0.7646893 | 1.6553217 | 0.9637824 | 1.0805078 | 1.0436599 | 1.1536056 | 1.3199021 |
| Phase-1 RCT-169 | 0.8365963 | 0.9174885 | 1.1174746 | 1.118112 | 0.9038318 | 0.9241813 | 0.9053956 |
| Pyruvate kinase, muscle | 1.0278003 | 1.3567251 | 1.2501168 | 0.9635986 | 1.0044435 | 1.0428089 | 1.0459358 |
| Nucleosome assembly protein | 0.8544565 | 0.9650805 | 0.9009547 | 0.6828291 | 0.6443229 | 1.1037319 | 0.7618153 |
| Phase-1 RCT-127 | 0.9829893 | 1.016016 | 1.1130027 | 0.9127799 | 1.0839665 | 1.2140782 | 1.0530958 |
| Iron-responsive element-binding protein | 1.1735312 | 1.3065581 | 1.2206095 | 1.0086617 | 1 | 0.8734257 | 1.166402 |
| Phase-1 RCT-72 | 0.4802456 | 1.2682431 | 1.4523982 | 0.9217782 | 0.8160432 | 0.8034738 | 0.9441825 |
| Phase-1 RCT-144 | 1.0888162 | 1.2009839 | 1.4401608 | 1.1579542 | 1.1704141 | 1.4015261 | 1.2898822 |
| Phase-1 RCT-242 | 1.3071805 | 1.0817133 | 1.5210397 | 0.8165627 | 0.8228148 | 0.9005507 | 0.9368073 |
| 17-beta hydroxysteroid dehydrogenase, | 1.0889945 | 0.9754195 | 0.7014058 | 0.3759749 | 0.394173 | 1.0195326 | 0.7033878 |
| type 2 | | | | | | | |
| Phase-1 RCT-70 | 1.0620308 | 0.9978299 | 1.3304162 | 0.9621158 | 0.9220058 | 0.8368836 | 1.0256853 |
| Ribosomal protein L13A | 0.6940989 | 0.4019599 | 0.393758 | 0.990426 | 1.0301706 | 1.3010312 | 1.2461061 |
| Cytochrome P450 2E1 | 0.9595326 | 1.3030417 | 0.7976719 | 0.7650495 | 1.0111758 | 0.7427582 | 1.0257722 |
| AT-3 | 1.074381 | 1.067379 | 1.1921375 | 0.7742634 | 0.9204636 | 0.9644585 | 0.8813297 |
| Phase-1 RCT-270 | 0.8534871 | 0.8087534 | 0.6133242 | 1.0632771 | 0.9553252 | 0.7021761 | 0.8454317 |
| Phase-1 RCT-123 | 1.214826 | 1.1031317 | 1.336416 | 0.9722635 | 1.0026666 | 1.0484949 | 0.9390423 |
| Alpha-2-macroglobulin, sequence 2 | 0.7706887 | 0.5716918 | 0.9547036 | 0.8112778 | 0.791016 | 0.7512797 | 1.0257317 |
| Matrix metalloproteinase-1 | 0.6770403 | 0.6632772 | 0.964018 | 0.9755983 | 1.0129212 | 0.9481756 | 1.5933832 |
| Beta-tubulin, class I | 2.015382 | 1.7375705 | 1.7845248 | 1.2701181 | 1.45566 | 1.2612182 | 1.1443676 |
| Phase-1 RCT-161 | 1.2933291 | 1.5659357 | 1.7074088 | 0.8985872 | 0.8265938 | 0.8613675 | 0.9533529 |
| 14-3-3 zeta | 1.7261057 | 1.8024441 | 1.6705579 | 1.4222497 | 1.2357888 | 1.2314328 | 1.4446098 |
| Stem cell factor | 1.0995482 | 0.9289839 | 0.8150577 | 1.0106875 | 0.9234433 | 0.9274489 | 0.7944489 |
| Macrophage inflammatory protein-2 alpha | 1.3763627 | 1.372251 | 1.4357891 | 0.8197635 | 0.9817379 | 1.1691839 | 1.0630679 |
| Thymidylate synthase | 1.2803364 | 1.2987072 | 1.3412137 | 0.7803205 | 0.8352646 | 0.9589863 | 1.0577105 |
| c-jun | 1.6319388 | 1.7008042 | 1.5959191 | 0.8109295 | 0.8299388 | 0.7547144 | 1.005337 |
| Superoxide dismutase Mn | 1.2036241 | 1.0403733 | 0.813362 | 1.225496 | 1.153862 | 1.2095511 | 1.2610024 |
| Phase-1 RCT-73 | 1.3502263 | 1.1141754 | 1.1966648 | 1.1238501 | 0.989459 | 1.0635642 | 1.0228536 |
| Macrophage inflammatory protein-1 alpha | 1.7505885 | 1.4021825 | 1.3475821 | 1.3000499 | 1.3030622 | 1.3687288 | 0.9140722 |
| Phase-1 RCT-214 | 1.273834 | 1.2565428 | 1.007372 | 1.0716561 | 1.0757092 | 0.9974599 | 0.9477713 |
| NADH-cytochrome b5 reductase | 0.900146 | 1.0819883 | 0.8834139 | 1.0321225 | 1.0650631 | 0.9352624 | 0.9715114 |
| Interleukin-1 beta | 0.8287858 | 0.6545473 | 0.9697684 | 0.8267312 | 0.8509211 | 0.93448 | 1.4084119 |
| NADP-dependent isocitrate | 1.0289357 | 0.8029274 | 0.7760947 | 1.2033482 | 1.2194312 | 1.0507338 | 0.9435595 |
| dehydrogenase, cytosolic | | | | | | | |
| Phase-1 RCT-289 | 1.0177592 | 0.6327985 | 0.6660202 | 1.1613065 | 0.9661787 | 0.9535328 | 1.0071944 |
| Glutamine synthetase | 0.9013749 | 0.9709448 | 1.1017735 | 1.0163764 | 1.0810827 | 0.8440934 | 1.266148 |
| Phase-1 RCT-182 | 0.6531485 | 0.7537136 | 0.8888111 | 1.1450417 | 1.2700547 | 1.1425049 | 1.0442497 |
| Ubiquitin conjugating enzyme (RAD 6 | 0.7973788 | 0.7220284 | 0.9165605 | 1.1488587 | 1.2802948 | 1.2934208 | 1.1191027 |
| homologue) | | | | | | | |
| Extracellular-signal-regulated kinase 1 | 1.5942118 | 1.2577571 | 0.7783185 | 0.8625887 | 0.7358346 | 0.66625 | 0.4305806 |
| Emerin | 1.3627053 | 0.9964275 | 0.8552358 | 1.0458349 | 1.098714 | 1.0966991 | 1.0511054 |
| Phase-1 RCT-78 | 1.0251043 | 1.1065596 | 1.0188948 | 0.9887805 | 1.0003217 | 1.088044 | 0.9763737 |
| Phase-1 RCT-212 | 1.2615035 | 0.6664614 | 0.8640643 | 0.9765258 | 1.0290979 | 0.9600404 | 0.902106 |
| Phase-1 RCT-168 | 0.9678366 | 0.7510443 | 0.7091709 | 0.9810729 | 0.9263077 | 0.9045349 | 0.8858248 |
| Lysyl hydroxylase | 0.9645497 | 1.0341085 | 1.1361778 | 0.9610851 | 0.9791007 | 1.1030238 | 1.1335691 |
| Phase-1 RCT-55 | 0.5181779 | 1.5971804 | 1.1171635 | 0.6851624 | 0.6775442 | 0.7020083 | 0.9551498 |
| MHC class I antigen RT1.A1(f) alpha- | 7.945626 | 2.1271622 | 4.445193 | 1.3666476 | 1.3390242 | 1.1774933 | 1.3408306 |
| chain | | | | | | | |
| Phase-1 RCT-40 | 0.706076 | 0.8084265 | 0.6748058 | 1.1213188 | 0.9700915 | 1.058306 | 0.9064109 |
| Cyclin G | 1.4946433 | 1.8336625 | 2.3144493 | 0.8454679 | 0.9870234 | 1.2621891 | 0.8842859 |
| Peroxisome proliferator activated receptor | 1.3740038 | 0.7963386 | 0.6964937 | 0.6550341 | 0.7680935 | 0.8708816 | 0.9201374 |
| gamma | | | | | | | |
| Phase-1 RCT-280 | 1.0263124 | 0.9802291 | 0.7488914 | 0.8659974 | 1.0292977 | 1.3050967 | 1.2253289 |
| D-dopachrome tautomerase | 1.0133225 | 0.6292806 | 1.0142854 | 0.9656038 | 0.9928342 | 1.1892623 | 0.861182 |
| Phase-1 RCT-287 | 1.1081758 | 1.0800303 | 0.9151021 | 1.2305877 | 1.1143019 | 1.1611038 | 0.9759294 |
| Phase-1 RCT-119 | 0.6646422 | 0.4547555 | 0.4630654 | 0.8092918 | 0.9079229 | 0.7854391 | 0.5237284 |
| Thioredoxin-1 (Trx1) | 0.6556689 | 0.3870278 | 0.5102024 | 1.0253559 | 0.9609112 | 1.1674062 | 1.2694361 |
| Phase-1 RCT-225 | 1.5049703 | 0.5220652 | 0.771519 | 1.2089598 | 2.2408903 | 2.0692575 | 1.1831052 |
| Protein kinase C alpha | 0.7583554 | 1.1558806 | 0.9539279 | 0.812669 | 0.75761 | 0.9160612 | 1.0347658 |
| Phase-1 RCT-87 | 0.6922123 | 0.9987872 | 0.9484658 | 1.0835038 | 0.9883269 | 1.1186347 | 1.0100102 |
| Phase-1 RCT-59 | 1.2067604 | 1.0969381 | 0.9339778 | 0.9202836 | 0.972082 | 0.8776463 | 1.0198094 |
| C4b-binding protein | 1.2370622 | 0.9423858 | 0.689967 | 1.1483116 | 1.1230686 | 1.4638089 | 0.7374078 |
| Phase-1 RCT-204 | 1.2013448 | 1.0251052 | 1.2979596 | 0.8495504 | 1.0157119 | 1.092109 | 1.0024334 |
| Carnitine palmitoyl-CoA transferase | 3.0166125 | 1.3086743 | 2.5445309 | 0.9583016 | 1.0299039 | 1.2254566 | 1.3172252 |
| 8-oxoguanine DNA glycosylase | 1.1661803 | 0.991453 | 1.0799471 | 0.6413288 | 0.8294979 | 0.9684202 | 0.9745966 |
| H-rev107 | 1.295059 | 1.1954513 | 1.8009129 | 0.8680386 | 0.97304 | 0.9757757 | 0.8726575 |
| Preproalbumin | 2.0477915 | 4.42191 | 1.4936846 | 1.1245829 | 1.0970465 | 0.8017859 | 1.0576568 |
| Phase-1 RCT-177 | 0.9886079 | 0.9092165 | 1.1839718 | 1.2160796 | 0.9896467 | 1.1506846 | 1.0038823 |
| Phase-1 RCT-110 | 1.3652238 | 1.1462941 | 1.5387638 | 0.9248425 | 0.8950687 | 0.9022322 | 0.9588538 |
| Phase-1 RCT-227 | 0.6490801 | 0.8303464 | 0.6902919 | 1.054546 | 0.926469 | 1.0279601 | 0.9999609 |
| Neuropeptide Y | 1.0514113 | 1.3207853 | 1.3800428 | 0.8571524 | 0.8817468 | 0.9113066 | 0.9171492 |
| Phase-1 RCT-277 | 0.874599 | 0.8847606 | 1.0283706 | 0.7696549 | 0.9175957 | 0.7710705 | 0.7928301 |
| Phase-1 RCT-229 | 1.3366885 | 1.2079084 | 1.3986844 | 0.8803108 | 0.8955528 | 0.9185297 | 0.8867984 |
| Phase-1 RCT-34 | 0.8140321 | 1.1975708 | 0.8327398 | 1.8537018 | 1.5218847 | 1.3066714 | 1.0601436 |
| Choline kinase | 1.7444603 | 0.87523 | 1.017467 | 1.1057991 | 1.0676453 | 0.7368851 | 1.0030779 |
| Phase-1 RCT-20 | 1.1501327 | 1.1773386 | 1.6918511 | 0.9927102 | 0.8999685 | 0.9086976 | 0.9491751 |
| Phase-1 RCT-248 | 1.1211339 | 0.6294681 | 0.8195482 | 0.910331 | 0.8343276 | 0.9607132 | 1.0496676 |
| | PBARB | PBARB | PBARB | PBARB | PBARB | PBARB | PHEN |
| Compound-Dose (2) | 20 | 20 | 20 | 80 | 80 | 80 | 20 |
| Animal Number (3) | 2621 | 2622 | 2623 | 2631 | 2632 | 2633 | 1321 |
| Liver Toxicity Necrosis Classification (4) | no | no | no | no | no | no | no |
| Gene Name (5) | | | | | | | |
| Gadd153 | 1.0247533 | 1.1156038 | 1.8162385 | 1.1323488 | 1.013142 | 1.1017112 | 1.088577 |
| Interferon related developmental regulator | 1.218587 | 1.0111693 | 0.9902828 | 1.4182678 | 1.2900181 | 1.3345567 | 0.9290552 |
| IFRD1 (PC4) | | | | | | | |
| ID-1 | 0.8794782 | 0.8796123 | 0.9079107 | 0.9065543 | 0.8957102 | 0.9198678 | 1.1059829 |
| Insulin-like growth factor binding protein 1 | 1.2372787 | 0.9915605 | 0.914017 | 1.1060444 | 0.8741062 | 1.2568069 | 1.0111321 |
| NIPK | 1.2191901 | 1.0742545 | 1.0185908 | 1.9312441 | 1.109624 | 1.3595613 | 0.9701211 |
| Phase-1 RCT-50 | 0.9545364 | 1.0219473 | 1.0604969 | 1.0029012 | 1.0034884 | 1.0515954 | 1.1630429 |
| Sarcoplasmic reticulum calcium ATPase | 0.9693735 | 0.923159 | 0.9708613 | 1.0011289 | 0.8673949 | 0.9556758 | 0.8371521 |
| Phase-1 RCT-207 | 1.0240494 | 1.1192498 | 0.9757444 | 1.0157868 | 1.0769681 | 1.0104657 | 1.0602763 |
| Acetyl-CoA carboxylase | 0.9915635 | 1.0080628 | 1.0103029 | 1.1043069 | 0.9921169 | 0.9982567 | 1.42627 |
| Cathepsin L, sequence 2 | 0.9652244 | 0.8663293 | 0.7091564 | 1.0257555 | 1.1249918 | 1.2587708 | 1.3667697 |
| Glucokinase | 0.7567006 | 0.7822213 | 0.9062271 | 0.7781191 | 0.7188006 | 0.6465153 | 0.7165667 |
| Gadd45 | 0.8857994 | 0.9784833 | 0.9524151 | 1.2099104 | 1.4333833 | 1.3959785 | 1.1212214 |
| Phase-1 RCT-18 | 0.9226731 | 0.9306577 | 0.960335 | 1.0258609 | 0.9328029 | 0.8859199 | 0.947854 |
| 3-beta-hydroxysteroid dehydrogenase | 1.0154197 | 1.0382729 | 0.9956063 | 0.90299 | 0.8881743 | 0.85048 | 2.663502 |
| (HSD3B1) | | | | | | | |
| Phase-1 RCT-221 | 1.1374173 | 0.9908309 | 1.0092374 | 1.1652727 | 0.8120102 | 0.9507652 | 0.897544 |
| Heme oxygenase | 0.6606793 | 0.6321205 | 0.8150649 | 0.8373669 | 0.6587765 | 1.088914 | 1.3839298 |
| Cyclin dependent kinase 4 | 1.3186461 | 1.1872873 | 1.0169244 | 0.9560511 | 0.9903225 | 1.1030264 | 1.3233958 |
| Phase-1 RCT-75 | 1.0575826 | 1.0405253 | 1.0261899 | 0.8805679 | 0.7565807 | 1.0823656 | 1.0436015 |
| Phase-1 RCT-65 | 1.0641862 | 1.0689392 | 1.0900856 | 1.0990281 | 1.0326957 | 1.0285457 | 0.973537 |
| Cytochrome P450 1A1 | 0.9889239 | 0.96308 | 1.047614 | 1.0025958 | 0.9881126 | 0.872875 | 0.9431733 |
| Peroxisomal multifunctional enzyme type II | 1.244917 | 1.2219814 | 1.0305682 | 1.206624 | 1.069516 | 1.3685039 | 1.0454307 |
| DNA topoisomerase I | 1.0416996 | 0.9055305 | 0.9579684 | 0.987969 | 1.0252639 | 0.8357293 | 1.0304797 |
| Focal adhesion kinase (pp125FAK) | 1.0369568 | 0.9042841 | 0.9072232 | 1.0071753 | 1.009655 | 0.9193827 | 0.6747646 |
| NGF-inducible anti-proliferative putative | 1.0100541 | 1.2219512 | 1.0425868 | 1.038099 | 0.8618004 | 1.0103569 | 0.609036 |
| secreted protein (PC3) | | | | | | | |
| Phase-1 RCT-82 | 0.9505523 | 0.9478775 | 1.0112163 | 0.9645225 | 0.9792423 | 0.8893371 | 0.9554222 |
| Senescence marker protein-30 | 0.8352284 | 0.7808923 | 0.9886797 | 0.7127075 | 0.5319546 | 0.5006512 | 0.8006672 |
| Melanoma-associated antigen ME491 | 0.9506882 | 0.8737901 | 1.3423421 | 1.0523821 | 1.0077443 | 1.3003591 | 0.90764 |
| Activating transcription factor 3 | 1.0668424 | 0.9287666 | 1.0418247 | 1.0840164 | 1.211955 | 0.9816461 | 1.149928 |
| Tryptophan hydroxylase | 1.0209187 | 0.949201 | 1.0252062 | 1.0661938 | 1.1336715 | 0.9603766 | 1.3700457 |
| Zinc finger protein | 1 | 1.2826557 | 0.9709471 | 1.1023464 | 1.0621527 | 1.0417241 | 0.9791908 |
| c-myc | 0.9038844 | 0.9761724 | 1.0651381 | 1.5922004 | 1.3051496 | 1.219585 | 1.1015917 |
| Cholesterol 7-alpha-hydroxylase P450 | 0.9315131 | 0.7244022 | 0.8114399 | 0.932146 | 1.3149862 | 2.6526217 | 1.0234778 |
| VII) | | | | | | | |
| Ferritin H-chain | 0.8830118 | 0.8099324 | 0.8515131 | 0.9591988 | 0.8788917 | 1.1192126 | 2.283105 |
| Phase-1 RCT-197 | 0.966273 | 0.8798428 | 0.8983957 | 1.12448 | 1.0357467 | 1.0149838 | 0.8972282 |
| Phase-1 RCT-179 | 1.0321391 | 1.0024121 | 0.8787526 | 0.9722288 | 0.9624073 | 1.0343386 | 1.2333217 |
| Phase-1 RCT-15 | 1.6196376 | 1.6505365 | 1.4982314 | 1.1655562 | 1.2942965 | 1.5130119 | 3.5297544 |
| Argininosuccinate lyase | 1.172175 | 0.8735927 | 1.2972374 | 0.6917074 | 1.1273544 | 1.9792662 | 1.0991616 |
| Phase-1 RCT-49 | 1.0190192 | 1.0418191 | 1.0214984 | 0.9735727 | 1.1563894 | 1.0542681 | 0.8601364 |
| Carbonic anhydrase III | 0.9347944 | 0.5103385 | 1.9155728 | 0.7125495 | 0.488099 | 0.3055625 | 1.7213516 |
| Proliferating cell nuclear antigen gene | 1.060467 | 1.0730444 | 1.060568 | 1.0893956 | 1.0287297 | 1.123764 | 0.9984905 |
| Integrin beta1 | 1.0090196 | 0.9852656 | 0.8468999 | 1.1624016 | 0.9317371 | 1.2336656 | 1.0932711 |
| Caspase 6 | 0.9526119 | 0.8699961 | 0.9094639 | 0.9849156 | 0.9677364 | 1.0682117 | 0.9862551 |
| Phase-1 RCT-116 | 0.9835418 | 0.9165409 | 0.9485145 | 1.1293076 | 1.0988579 | 1.1542474 | 0.968475 |
| Phase-1 RCT-109 | 1.0482209 | 1.0670625 | 1.1010877 | 0.7723628 | 0.8066127 | 1.0653814 | 0.971308 |
| Phase-1 RCT-71 | 1.0041296 | 1.0837991 | 1.0497049 | 0.9037051 | 0.8358902 | 0.9790472 | 0.7347947 |
| Elongation factor-1 alpha | 0.9791168 | 0.9765573 | 0.96506 | 0.962611 | 0.9845825 | 1.1908307 | 0.8647324 |
| Phase-1 RCT-169 | 0.8210536 | 0.8587768 | 0.9560475 | 1.2024158 | 0.9100347 | 0.6837238 | 1.0000807 |
| Pyruvate kinase, muscle | 1.09203 | 1.0301634 | 0.8849154 | 0.9849927 | 1.0208664 | 1.1916095 | 1.2391573 |
| Nucleosome assembly protein | 0.6923288 | 0.818627 | 1.1285776 | 0.9166316 | 1.1111679 | 0.9782622 | 1.4712149 |
| Phase-1 RCT-127 | 0.8960849 | 1.0099537 | 0.8105291 | 1.023065 | 1.1849915 | 1.0761226 | 1.1731942 |
| Iron-responsive element-binding protein | 0.9576453 | 1.064835 | 0.9665768 | 1.170347 | 1.0641296 | 1.0913563 | 0.7253452 |
| Phase-1 RCT-72 | 1.3333243 | 1.2053462 | 1.3740662 | 0.9557113 | 0.9778121 | 0.8497456 | 1.1133952 |
| Phase-1 RCT-144 | 1.0576887 | 1.1004744 | 0.9508874 | 0.9757937 | 0.8046352 | 1.0661235 | 0.9991027 |
| Phase-1 RCT-242 | 1.0072488 | 1.0268921 | 1.0261354 | 1.1014955 | 1.0172472 | 0.9477395 | 1.0337737 |
| 17-beta hydroxysteroid dehydrogenase, | 0.4654273 | 0.5428265 | 1.7431762 | 0.7901057 | 1.1655952 | 0.8895919 | 1.8514342 |
| type 2 | | | | | | | |
| Phase-1 RCT-70 | 0.9713092 | 0.8756042 | 0.9302869 | 0.947043 | 0.8712532 | 0.9054014 | 1.0029447 |
| Ribosomal protein L13A | 0.9581491 | 1.0040352 | 0.871541 | 0.7609926 | 0.7667542 | 0.9259638 | 1.4680055 |
| Cytochrome P450 2E1 | 0.4976948 | 0.6841765 | 0.6348579 | 0.689052 | 0.9165082 | 0.7095984 | 1.0474955 |
| AT-3 | 0.9128751 | 0.8667017 | 0.832455 | 0.9636016 | 0.9138482 | 0.9201775 | 0.9486357 |
| Phase-1 RCT-270 | 1.1571403 | 1.24947 | 1.292048 | 1.0085881 | 1.2976952 | 1.1093677 | 0.6594982 |
| Phase-1 RCT-123 | 0.9714688 | 1.0018218 | 0.9965871 | 1.0039306 | 1.0140212 | 0.8933343 | 1.0073142 |
| Alpha-2-macroglobulin, sequence 2 | 0.9867714 | 0.8740516 | 0.8331696 | 0.7777333 | 0.9252778 | 1.1062748 | 0.5449995 |
| Matrix metalloproteinase-1 | 0.9982247 | 0.9132081 | 0.8350927 | 0.9575033 | 0.8918539 | 1.035769 | 1.8189772 |
| Beta-tubulin, class I | 1.5627121 | 1.3392687 | 1.2407404 | 0.9569603 | 0.7458804 | 0.9381945 | 0.9373528 |
| Phase-1 RCT-161 | 0.9972942 | 0.8001935 | 1.0658565 | 1.0742984 | 1.0210888 | 0.9292739 | 1.243147 |
| 14-3-3 zeta | 1.160863 | 1.0961487 | 1.005734 | 1.2140929 | 0.9284404 | 1.1080934 | 1.12982 |
| Stem cell factor | 1.0127119 | 0.843593 | 0.9586356 | 0.8686059 | 0.6594958 | 0.678501 | 0.8883727 |
| Macrophage inflammatory protein-2 alpha | 0.9935693 | 1.1352926 | 1.0100117 | 1.2426208 | 1.0782763 | 1.2450438 | 1.0706942 |
| Thymidylate synthase | 0.6528539 | 0.7385396 | 1.001714 | 0.7001616 | 0.7986652 | 0.6894252 | 0.9424411 |
| c-jun | 0.8332413 | 0.8547956 | 0.8272472 | 1.520916 | 1.056755 | 0.9601368 | 1.0440879 |
| Superoxide dismutase Mn | 1.1442118 | 1.1333251 | 1.084696 | 1.1687912 | 1.1475984 | 1.24016 | 1.071405 |
| Phase-1 RCT-73 | 0.9447477 | 1.0407711 | 0.9823728 | 1.053366 | 0.9918321 | 1.0223083 | 1.2394292 |
| Macrophage inflammatory protein-1 alpha | 0.9784293 | 1.0254483 | 0.9944513 | 1.0255982 | 1.0331396 | 1.023854 | 1.1438928 |
| Phase-1 RCT-214 | 0.8327148 | 1.0279021 | 0.8965928 | 0.8370789 | 0.7112196 | 0.7855712 | 1.0071084 |
| NADH-cytochrome b5 reductase | 1.0007405 | 1.2125204 | 1.1356368 | 0.85391 | 1.1467522 | 0.9534244 | 1.0177183 |
| Interleukin-1 beta | 1.0288243 | 1.01339 | 1.0355874 | 1.0837005 | 1.0044345 | 1.087687 | 1.3022223 |
| NADP-dependent isocitrate | 1.1168 | 0.9844763 | 1.0626094 | 1.031399 | 0.8522132 | 0.7667258 | 0.9475601 |
| dehydrogenase, cytosolic | | | | | | | |
| Phase-1 RCT-289 | 0.9590958 | 1.0390788 | 1.0353521 | 0.9744958 | 1.0383286 | 1.0117488 | 0.8072727 |
| Glutamine synthetase | 0.6483477 | 0.6585429 | 0.8646696 | 0.6893234 | 0.6080985 | 0.7349125 | 0.8477446 |
| Phase-1 RCT-182 | 1.025232 | 0.8328319 | 1.0800527 | 0.8492497 | 1.0134063 | 0.9788052 | 0.5449424 |
| Ubiquitin conjugating enzyme (RAD 6 | 0.844881 | 0.9887558 | 0.9719559 | 0.7648401 | 0.7820481 | 0.9093642 | 0.9426815 |
| homologue) | | | | | | | |
| Extracellular-signal-regulated kinase 1 | 1.2613025 | 0.6866677 | 2.0806813 | 0.7588863 | 0.5457937 | 0.3801158 | 1.2615129 |
| Emerin | 1.0163149 | 0.9938058 | 0.9186198 | 0.9338301 | 0.9217837 | 1.1504043 | 1.0827245 |
| Phase-1 RCT-78 | 1.1788602 | 1.0750444 | 1.1497099 | 1.2684222 | 1.2497126 | 1.2598948 | 1.3762392 |
| Phase-1 RCT-212 | 0.8342893 | 0.9532291 | 1.0466273 | 0.878644 | 0.9374952 | 1.068466 | 0.8163214 |
| Phase-1 RCT-168 | 0.969666 | 0.9852228 | 1.0424283 | 0.8560921 | 0.8773232 | 0.7999534 | 0.8123817 |
| Lysyl hydroxylase | 0.7966217 | 0.7542306 | 0.8714476 | 0.8321081 | 0.7433102 | 0.8332204 | 0.8289194 |
| Phase-1 RCT-55 | 0.9814958 | 0.9040124 | 1.0063313 | 0.5655928 | 0.5382698 | 0.7552479 | 0.8525771 |
| MHC class I antigen RT1.A1(f) alpha- | 1.227902 | 1.2196568 | 1.2059363 | 1.2455714 | 1.0230098 | 1.1611935 | 1.0786797 |
| chain | | | | | | | |
| Phase-1 RCT-40 | 1.0762416 | 0.8885903 | 1.0599844 | 1.0222068 | 0.934812 | 0.856204 | 0.8972715 |
| Cyclin G | 1.0881777 | 1.07105 | 1.13835 | 0.9985842 | 0.9813076 | 1.1109604 | 1.0575448 |
| Peroxisome proliferator activated receptor | 0.6521976 | 0.6946006 | 0.5081223 | 0.7181029 | 0.7923243 | 0.7027285 | 0.8680874 |
| gamma | | | | | | | |
| Phase-1 RCT-280 | 1.0432798 | 0.9739839 | 0.9826619 | 1.1020333 | 1.357472 | 1.0601299 | 1.0689518 |
| D-dopachrome tautomerase | 1.1326549 | 0.9455876 | 1.3615543 | 0.7974365 | 0.8351667 | 1.1992284 | 0.6443265 |
| Phase-1 RCT-287 | 0.9744274 | 0.8976533 | 0.8774621 | 0.9916574 | 0.8884593 | 0.9526708 | 0.996015 |
| Phase-1 RCT-119 | 0.8747509 | 0.6412503 | 0.995261 | 0.6659082 | 1.0657198 | 0.991219 | 0.8207868 |
| Thioredoxin-1 (Trx1) | 1.0633303 | 1.0357592 | 0.9787087 | 0.9552776 | 0.9095519 | 1.229932 | 0.9310074 |
| Phase-1 RCT-225 | 1.1095155 | 0.7993423 | 0.9495663 | 0.7460682 | 0.8009132 | 0.6882154 | 0.8858112 |
| Protein kinase C alpha | 1.0667315 | 1.1513287 | 1.0755838 | 1.0558159 | 0.9942247 | 1.0358374 | 0.8255222 |
| Phase-1 RCT-87 | 0.9951307 | 1.0005308 | 1.06798 | 0.9291108 | 0.946898 | 1.0964673 | 1.0775862 |
| Phase-1 RCT-59 | 0.9094712 | 1.1253064 | 1.0806004 | 0.9966924 | 1.0882748 | 0.981392 | 1.0893652 |
| C4b-binding protein | 0.7613713 | 0.7083098 | 0.7911043 | 0.9865399 | 0.857442 | 0.7948673 | 0.7314538 |
| Phase-1 RCT-204 | 1.0739672 | 1.0435286 | 1.1053118 | 0.9269427 | 1.0444175 | 1.0060909 | 0.9857124 |
| Carnitine palmitoyl-CoA transferase | 0.9901516 | 0.9969968 | 0.8743483 | 1.1339796 | 1.0564288 | 1.1917455 | 0.9961065 |
| 8-oxoguanine DNA glycosylase | 1.015807 | 0.9513495 | 0.8438926 | 1.0248638 | 0.945156 | 0.9783639 | 0.9924049 |
| H-rev107 | 0.9595378 | 0.9839644 | 0.8788559 | 0.887351 | 0.9573618 | 0.9004516 | 0.9248664 |
| Preproalbumin | 0.9703915 | 1.168751 | 1.0977635 | 1.0383672 | 1.3389838 | 1.0649399 | 7.166313 |
| Phase-1 RCT-177 | 1.1120609 | 1.0777332 | 0.8891019 | 1.0436306 | 0.9575945 | 1.1552696 | 1.736098 |
| Phase-1 RCT-110 | 0.9793769 | 0.949418 | 1.0365926 | 1.0343928 | 1.0189517 | 0.8946505 | 1.0768346 |
| Phase-1 RCT-227 | 1.2663277 | 1.036791 | 1.0844696 | 1.3995479 | 1.3316028 | 1.4833747 | 1.6418245 |
| Neuropeptide Y | 0.9786442 | 1.0256027 | 1.0152079 | 1.0010686 | 1.0777713 | 1.0202055 | 0.847755 |
| Phase-1 RCT-277 | 0.9113821 | 1.2384039 | 0.7306898 | 0.8449046 | 0.7626158 | 0.8432234 | 1.0428561 |
| Phase-1 RCT-229 | 0.9953741 | 0.9833824 | 1.0091054 | 1.0281692 | 1.0936998 | 1.0159459 | 1.3125952 |
| Phase-1 RCT-34 | 1.0585179 | 1.0462052 | 1.1479174 | 1.0504084 | 0.8275412 | 1.0151272 | 1.1258456 |
| Choline kinase | 1.1568407 | 0.9518518 | 1.0471545 | 1.0252645 | 0.9372641 | 1.0791435 | 0.8775187 |
| Phase-1 RCT-20 | 0.9957871 | 1.0762982 | 0.976711 | 1.0082656 | 0.9874384 | 0.9612739 | 1.4368775 |
| Phase-1 RCT-248 | 0.9353126 | 1.0399693 | 1.0813643 | 0.8883298 | 0.9163885 | 0.93492 | 0.9715969 |
| | PHEN | PHEN | PHEN | PHEN | PHEN | PEG | PEG |
| Compound-Dose (2) | 20 | 20 | 80 | 80 | 80 | 5000 | 5000 |
| Animal Number (3) | 1322 | 1323 | 1331 | 1332 | 1333 | 141 | 142 |
| Liver Toxicity Necrosis Classification (4) | no | no | no | no | no | no | no |
| Gene Name (5) | | | | | | | |
| Gadd153 | 1.1995702 | 1.2666667 | 1.1693778 | 0.9951903 | 1.1755555 | 0.8387576 | 0.9266505 |
| Interferon related developmental regulator | 0.7158182 | 0.879222 | 0.5316183 | 0.7354487 | 0.7217255 | 1.0183065 | 1.1218348 |
| IFRD1 (PC4) | | | | | | | |
| ID-1 | 1.2986774 | 1.3141364 | 1.7983679 | 1.2937155 | 1.3552673 | 0.8880639 | 0.8481219 |
| Insulin-like growth factor binding protein 1 | 0.9769619 | 1.0899327 | 1.1592891 | 1.1772097 | 1.1429492 | 1.089308 | 1.2422513 |
| NIPK | 0.7229675 | 1.0878652 | 1.7452253 | 1.2895318 | 1.5168066 | 0.817279 | 0.796863 |
| Phase-1 RCT-50 | 0.9504001 | 1.4580586 | 1.4430426 | 1.6234862 | 1.1987605 | 1.0434972 | 0.9314718 |
| Sarcoplasmic reticulum calcium ATPase | 1.0866649 | 0.8874294 | 0.6734563 | 0.8105515 | 0.9668189 | 1.0033226 | 1.0688877 |
| Phase-1 RCT-207 | 0.8443131 | 1.1951059 | 1.2575887 | 1.5198549 | 1.1839384 | 0.9509807 | 0.966844 |
| Acetyl-CoA carboxylase | 0.7682783 | 0.8757211 | 0.7093813 | 1.0590501 | 1.062048 | 0.9868784 | 0.9225375 |
| Cathepsin L, sequence 2 | 1.2381506 | 1.1990142 | 1.0164602 | 1.1912088 | 1.1516088 | 1.6093849 | 1.130862 |
| Glucokinase | 1.7143683 | 0.8938666 | 0.4342305 | 0.4287781 | 0.5431609 | 0.671631 | 0.7289248 |
| Gadd45 | 0.9801576 | 1.5476843 | 1.5847269 | 1.9324737 | 1.4866054 | 1.2706566 | 1.1800238 |
| Phase-1 RCT-18 | 0.9099535 | 0.8276979 | 0.795247 | 0.7866883 | 0.9973713 | 1.0223086 | 1.0343493 |
| 3-beta-hydroxysteroid dehydrogenase | 1.4742254 | 1.5543119 | 1.699762 | 2.792885 | 2.4521976 | 0.8011006 | 0.8288512 |
| (HSD3B1) | | | | | | | |
| Phase-1 RCT-221 | 1.0223651 | 0.8370827 | 0.6434123 | 0.8528887 | 0.7979116 | 0.8453091 | 0.7318186 |
| Heme oxygenase. | 0.9914061 | 1.4129506 | 1.9575106 | 2.1336405 | 2.64749 | 1.3145466 | 1.9329306 |
| Cyclin dependent kinase 4 | 1.0845207 | 1.5233209 | 1.4934033 | 1.2163558 | 1.2717952 | 0.7764615 | 0.8351604 |
| Phase-1 RCT-75 | 0.8215829 | 0.9922675 | 1.606634 | 1.1915759 | 1.1472889 | 0.9045126 | 0.8141701 |
| Phase-1 RCT-65 | 0.9196061 | 0.9188057 | 0.6280922 | 0.9214798 | 0.9800039 | 0.8353096 | 0.7819619 |
| Cytochrome P450 1A1 | 1.2237241 | 1.1587161 | 2.5097914 | 1.8488467 | 1.0511912 | 1.1915764 | 1.070636 |
| Peroxisomal multifunctional enzyme type II | 1.119119 | 0.8988622 | 0.9075171 | 1.2651827 | 1.0011868 | 1.063794 | 1.0823125 |
| DNA topoisomerase I | 0.9989806 | 0.637692 | 0.4090469 | 0.5469732 | 0.5962006 | 1.1216801 | 1.2448287 |
| Focal adhesion kinase (pp125FAK) | 1.202056 | 0.5352079 | 1.3538481 | 0.6586257 | 0.7128604 | 1.221897 | 1.1863109 |
| NGF-inducible anti-proliferative putative | 0.5273156 | 0.5948268 | 0.4412934 | 0.4905701 | 0.566941 | 0.935155 | 1.3151006 |
| vsecreted protein (PC3) | | | | | | | |
| Phase-1 RCT-82 | 0.9749765 | 0.9662414 | 1.2260474 | 1.2489845 | 0.9289853 | 0.947355 | 0.9668568 |
| Senescence marker protein-30 | 1.1264855 | 0.5692905 | 0.1897511 | 0.3036077 | 0.4663493 | 0.4030454 | 0.9013601 |
| Melanoma-associated antigen ME491 | 1.1525635 | 1.1810694 | 1.386257 | 1.0466396 | 0.9930644 | 1.1365267 | 0.9511741 |
| Activating transcription factor 3 | 0.801557 | 1.266311 | 1.2442491 | 1.466224 | 1.3588183 | 0.9888876 | 0.9296579 |
| Tryptophan hydroxylase | 1.0670235 | 1.2903811 | 0.9313992 | 1.3320335 | 1.0765119 | 0.7785382 | 0.8367071 |
| Zinc finger protein | 0.8665585 | 0.7154369 | 0.6947609 | 0.6660246 | 0.7472996 | 1.0505089 | 1.1622735 |
| c-myc | 0.8651384 | 1.4398625 | 2.1121607 | 1.777207 | 1.3777273 | 1.3683014 | 1.1729277 |
| Cholesterol 7-alpha-hydroxylase P450 | 1.5508661 | 1.3181542 | 1.4044908 | 0.6993446 | 0.9411566 | 0.6432459 | 0.7116729 |
| VII) | | | | | | | |
| Ferritin H-chain | 1.0329417 | 1.7181236 | 1.2340729 | 1.8780612 | 1.9226501 | 1.1510612 | 1.2411466 |
| Phase-1 RCT-197 | 0.8365573 | 0.9335776 | 0.7822934 | 0.834303 | 0.7850354 | 1.1328113 | 0.915529 |
| Phase-1 RCT-179 | 1.0883875 | 1.0033563 | 1.0451742 | 0.9882843 | 0.8716877 | 1.0124098 | 0.9632288 |
| Phase-1 RCT-15 | 1.0651319 | 2.3134992 | 1.634768 | 2.171104 | 2.1263123 | 0.9981898 | 1.0709243 |
| Argininosuccinate lyase | 1.2501523 | 0.9446797 | 0.4604164 | 0.9859073 | 0.7193275 | 1.2533381 | 1.0671562 |
| Phase-1 RCT-49 | 0.9219841 | 0.8275436 | 1.4815218 | 0.987803 | 0.9926372 | 1.0732799 | 1.065717 |
| Carbonic anhydrase III | 1.1666462 | 0.7175977 | 0.5098476 | 0.6897191 | 0.6762102 | 0.5153954 | 0.8356118 |
| Proliferating cell nuclear antigen gene | 1.1285015 | 1.2494142 | 1.2121432 | 0.8818202 | 1.1517814 | 1.2454054 | 1.1725035 |
| Integrin beta1 | 1.0416279 | 1.4231727 | 1.6306949 | 2.2487478 | 1.6656935 | 1.0033205 | 1.1197953 |
| Caspase 6 | 1.0946655 | 1.126829 | 1.0405338 | 1.0520545 | 1.203263 | 1.0329467 | 1.0660926 |
| Phase-1 RCT-116 | 0.8370483 | 1.4959221 | 1.1039135 | 1.6731662 | 1.8138875 | 0.6587553 | 0.708074 |
| Phase-1 RCT-109 | 0.8813652 | 0.9132051 | 1.1370656 | 1.0917772 | 1.1954769 | 0.8889813 | 0.8769499 |
| Phase-1 RCT-71 | 1.0699549 | 1.0491813 | 0.9887803 | 1.0521528 | 0.9431556 | 0.987296 | 1.0058271 |
| Elongation factor-1 alpha | 1.053152 | 0.5931522 | 0.4009376 | 0.5464143 | 0.8542034 | 0.9380279 | 0.9433216 |
| Phase-1 RCT-169 | 0.8884656 | 1.0351144 | 0.6997022 | 0.8865229 | 0.7300046 | 0.8794184 | 0.8222106 |
| Pyruvate kinase, muscle | 1.0347295 | 1.1922473 | 1.1538663 | 1.2490314 | 1.1450936 | 1.1946273 | 0.9618976 |
| Nucleosome assembly protein | 2.8556898 | 1.4146132 | 1.021456 | 1.4704812 | 1.208362 | 1.625757 | 1.770932 |
| Phase-1 RCT-127 | 0.8746264 | 0.8630269 | 1.593747 | 1.0895053 | 1.2648574 | 1.1041766 | 0.970284 |
| Iron-responsive element-binding protein | 0.9828642 | 0.8886303 | 0.8545156 | 0.6590506 | 0.8462193 | 0.8643937 | 1.1057422 |
| Phase-1 RCT-72 | 0.9821956 | 1.1925652 | 1.1126335 | 1.1997893 | 1.08982 | 0.8639917 | 0.9698886 |
| Phase-1 RCT-144 | 1.1174304 | 0.9827853 | 1.65814 | 1.1553274 | 1.1185299 | 0.9038513 | 0.9301565 |
| Phase-1 RCT-242 | 0.9906266 | 1.1379157 | 1.7740561 | 1.4139427 | 1.1897812 | 1.1481904 | 1.3181925 |
| 17-beta hydroxysteroid dehydrogenase, | 6.0886645 | 1.4235861 | 1.0183786 | 1.4620717 | 1.1201662 | 2.2176101 | 1.8636501 |
| type 2 | | | | | | | |
| Phase-1 RCT-70 | 0.7110149 | 0.8536215 | 1.0682658 | 1.136257 | 1.0389031 | 0.991398 | 1.0775403 |
| Ribosomal protein L13A | 0.9081181 | 1.2264652 | 0.9733213 | 1.2456899 | 1.5655851 | 0.8886427 | 0.8132256 |
| Cytochrome P450 2E1 | 1.8851967 | 1.6501358 | 1.4741321 | 1.2321445 | 0.9003852 | 0.8787164 | 0.9310749 |
| AT-3 | 1.0689591 | 0.8618068 | 1.4422562 | 0.8794884 | 0.8734754 | 1.0436265 | 1.1171165 |
| Phase-1 RCT-270 | 0.9437037 | 0.6276115 | 0.3825029 | 0.4300135 | 0.5249834 | 0.8058478 | 0.9003498 |
| Phase-1 RCT-123 | 0.9902706 | 1.1301395 | 1.5063019 | 1.1142502 | 1.0285412 | 1.0402423 | 1.0472378 |
| Alpha-2-macroglobulin, sequence 2 | 1.2101953 | 0.7218121 | 0.3160206 | 0.4987905 | 0.4575886 | 0.9841508 | 1.0075958 |
| Matrix metalloproteinase-1 | 1.090431 | 1.562952 | 1.2152635 | 1.9331468 | 1.7775359 | 1.1864791 | 1.1477245 |
| Beta-tubulin, class I | 0.5695431 | 1.153271 | 1.0983839 | 1.133395 | 1.1492376 | 0.56266 | 0.5676826 |
| Phase-1 RCT-161 | 0.919584 | 1.3384517 | 1.3796713 | 1.5315013 | 1.2478894 | 0.7341741 | 0.7539306 |
| 14-3-3 zeta | 0.9454805 | 1.4821098 | 1.4955721 | 1.4282246 | 1.355797 | 0.7670581 | 0.8500257 |
| Stem cell factor | 1.0385752 | 0.8941948 | 0.5495268 | 0.5056059 | 0.732731 | 0.4953291 | 0.9225019 |
| Macrophage inflammatory protein-2 alpha | 1.2533404 | 1.3295642 | 1.456981 | 1.7901107 | 1.3821354 | 2.942998 | 1.9564955 |
| Thymidylate synthase | 1.1756895 | 1.402874 | 1.7747816 | 1.1288302 | 1.3272288 | 1.1043297 | 1.1647047 |
| c-jun | 1.0323354 | 1.3379072 | 1.183718 | 1.7598337 | 1.3914863 | 0.9158442 | 0.9021527 |
| Superoxide dismutase Mn | 0.8789546 | 1.118839 | 0.941356 | 1.1316545 | 1.2891518 | 1.4026673 | 1.1485211 |
| Phase-1 RCT-73 | 0.9733691 | 1.240843 | 0.6823995 | 1.2640795 | 1.0449661 | 1.0212553 | 1.0926237 |
| Macrophage inflammatory protein-1 alpha | 1.0980508 | 1.252087 | 1.3338223 | 1.1260813 | 1.1837777 | 1.2980744 | 0.9568259 |
| Phase-1 RCT-214 | 0.6219892 | 0.799197 | 0.4768786 | 0.7030253 | 0.9870643 | 0.7792628 | 0.9839965 |
| NADH-cytochrome b5 reductase | 0.6694308 | 0.8826934 | 0.7491446 | 0.7002627 | 0.7156898 | 0.8676338 | 0.9999577 |
| Interleukin-1 beta | 1.163902 | 1.5790772 | 1.3750653 | 1.676032 | 1.6271116 | 1.2434183 | 1.1107947 |
| NADP-dependent isocitrate | 0.8442578 | 0.7834249 | 1.101536 | 0.6220579 | 0.6937524 | 0.890878 | 1.0579951 |
| dehydrogenase, cytosolic | | | | | | | |
| Phase-1 RCT-289 | 1.0415077 | 0.863539 | 0.7777567 | 0.7249048 | 0.7192993 | 0.9498252 | 0.9755985 |
| Glutamine synthetase | 1.338546 | 0.7741242 | 0.5056679 | 0.6727383 | 0.5808148 | 1.0054841 | 1.0304278 |
| Phase-1 RCT-182 | 1.3869008 | 0.6491612 | 0.4470218 | 0.5281956 | 0.4435347 | 1.142516 | 1.1821607 |
| Ubiquitin conjugating enzyme (RAD 6 | 1.0518057 | 0.6564333 | 0.6710243 | 0.6205068 | 0.6019797 | 1.1180228 | 1.0648289 |
| homologue) | | | | | | | |
| Extracellular-signal-regulated kinase 1 | 1.2912947 | 1.0958047 | 0.9553338 | 0.8128643 | 0.8496453 | 0.5681351 | 0.869917 |
| Emerin | 0.8483466 | 0.9675309 | 1.2920823 | 0.9046095 | 1.0784153 | 0.9303685 | 0.9903262 |
| Phase-1 RCT-78 | 1.0234376 | 1.1967646 | 0.9696712 | 1.3268954 | 1.1507969 | 1.0116206 | 1 |
| Phase-1 RCT-212 | 0.8073475 | 0.9550391 | 0.9620966 | 0.9316597 | 0.8189821 | 0.8807135 | 0.8014727 |
| Phase-1 RCT-168 | 1.1742411 | 0.6424666 | 1.0867578 | 0.6763883 | 0.7276874 | 0.9039989 | 0.9983012 |
| Lysyl hydroxylase | 1.3614123 | 0.7748991 | 0.732631 | 0.6764639 | 0.5621877 | 0.9632765 | 1.0545431 |
| Phase-1 RCT-55 | 1.2611818 | 0.8335255 | 0.5310882 | 0.9505026 | 1.1775051 | 0.6549422 | 0.5763462 |
| MHC class I antigen RT1.A1(f) alpha- | 0.7045261 | 1.2992707 | 0.9309924 | 1.11526 | 1.0888743 | 0.7948824 | 0.6923142 |
| chain | | | | | | | |
| Phase-1 RCT-40 | 0.9037123 | 0.7236282 | 0.454836 | 0.6787182 | 0.9790198 | 1.1825911 | 1.1886967 |
| Cyclin G | 1.0889081 | 1.3929912 | 1.7177427 | 1.5048895 | 1.3911839 | 1.2799363 | 0.7984356 |
| Peroxisome proliferator activated receptor | 1.1782305 | 1.2805393 | 1.9910777 | 0.8905022 | 1.0774273 | 1.1057912 | 1.2795272 |
| gamma | | | | | | | |
| Phase-1 RCT-280 | 0.8569488 | 0.7204538 | 0.4489199 | 0.7083872 | 1.0215383 | 1.114795 | 1.305788 |
| D-dopachrome tautomerase | 1.287664 | 0.6231496 | 1.9025705 | 1.5083064 | 0.9767114 | 0.7560414 | 0.9334472 |
| Phase-1 RCT-287 | 0.8735367 | 0.8285056 | 0.9576961 | 0.9248911 | 0.9330146 | 0.8642831 | 1.0928617 |
| Phase-1 RCT-119 | 1.1139419 | 0.8021287 | 0.45551 | 1.0477946 | 0.9370368 | 1.0710961 | 0.8772182 |
| Thioredoxin-1 (Trx1) | 0.839216 | 0.984234 | 0.8256128 | 0.8813368 | 0.9318594 | 1.1696607 | 1.1778405 |
| Phase-1 RCT-225 | 0.5634041 | 0.9388137 | 1.0543327 | 0.9223462 | 0.9357133 | 0.6076081 | 0.6826361 |
| Protein kinase C alpha | 0.9325432 | 0.8362094 | 1.1870774 | 0.67207 | 0.9010476 | 0.8571161 | 1.0764468 |
| Phase-1 RCT-87 | 0.972421 | 0.949874 | 0.8755049 | 0.8561884 | 0.848228 | 0.9625322 | 1.1180357 |
| Phase-1 RCT-59 | 1.024917 | 0.9174081 | 1.0365509 | 1.0070639 | 1.0950058 | 1.0884624 | 1.059504 |
| C4b-binding protein | 0.9477621 | 0.6890364 | 0.6879969 | 0.5242738 | 0.6306124 | 1.267755 | 1.3542994 |
| Phase-1 RCT-204 | 0.9619961 | 1.1748432 | 1.6099974 | 1.1732575 | 0.9735441 | 0.9551877 | 1.019032 |
| Carnitine palmitoyl-CoA transferase | 1.0591898 | 1.1588184 | 1.4112753 | 1.494711 | 1.1884104 | 1.2322675 | 1.0434328 |
| 8-oxoguanine DNA glycosylase | 0.9259135 | 1.2338805 | 1.3476398 | 1.0543523 | 1.1158218 | 1.1057494 | 0.9160441 |
| H-rev107 | 1.0113721 | 1.044305 | 1.6344833 | 1.027987 | 1.0393873 | 1.0143219 | 0.9367648 |
| Preproalbumin | 0.949587 | 3.6556916 | 2.12044 | 3.6879866 | 3.1822946 | 0.9067547 | 1.0171164 |
| Phase-1 RCT-177 | 0.9296984 | 1.2436886 | 0.6319693 | 1.1920685 | 1.0476997 | 0.9754508 | 0.9712356 |
| Phase-1 RCT-110 | 0.9645275 | 0.9295462 | 1.1417056 | 0.9694704 | 0.7935793 | 1.0572066 | 0.9150135 |
| Phase-1 RCT-227 | 1.1723293 | 1.2510451 | 0.9573796 | 1.4053389 | 1.1711311 | 1.2820278 | 1.0683969 |
| Neuropeptide Y | 1.0453558 | 0.6774383 | 0.6381983 | 0.7242649 | 0.5965275 | 1.030898 | 1.3187762 |
| Phase-1 RCT-277 | 1.2076888 | 1.0210714 | 0.3743126 | 0.4424869 | 0.3740071 | 0.9855167 | 1.4141408 |
| Phase-1 RCT-229 | 1.0267891 | 1.3200926 | 1.1809031 | 1.3371431 | 1.1407516 | 0.9758002 | 0.9413409 |
| Phase-1 RCT-34 | 1.0651885 | 1.1086041 | 0.9133645 | 1.2194682 | 1.2850326 | 0.8911597 | 0.8837652 |
| Choline kinase | 1.0726646 | 1.1063521 | 1.0356432 | 0.9921918 | 0.9167244 | 0.7504605 | 0.8615052 |
| Phase-1 RCT-20 | 1.0848695 | 1.5617558 | 1.5191047 | 1.9559133 | 1.7658793 | 0.9735481 | 0.9000952 |
| Phase-1 RCT-248 | 0.9098299 | 0.9321331 | 1.6620961 | 1.1077862 | 1.1129133 | 0.9659903 | 0.9686536 |
| | PEG | PUR | PUR | PUR | PUR | PUR | PUR |
| Compound-Dose (2) | 5000 | 38 | 38 | 38 | 150 | 150 | 150 |
| Animal Number (3) | 143 | 21 | 22 | 23 | 31 | 32 | 33 |
| Liver Toxicity Necrosis Classification (4) | no | no | no | no | no | no | no |
| Gene Name (5) | | | | | | | |
| Gadd153 | 0.904284 | 0.7132081 | 0.7535775 | 0.7163988 | 1.0073354 | 1.3829072 | 1.2740602 |
| Interferon related developmental regulator | 1 | 0.9965479 | 1.3189136 | 1.3860381 | 0.9613614 | 0.8344091 | 0.8722844 |
| IFRD1 (PC4) | | | | | | | |
| ID-1 | 0.866928 | 0.9742351 | 0.8679341 | 0.8400168 | 0.8937391 | 1.0599697 | 0.9311806 |
| Insulin-like growth factor binding protein 1 | 0.9622701 | 0.9561014 | 0.9071662 | 1.0404902 | 1.4577785 | 1.0167514 | 1.035789 |
| NIPK | 0.8086367 | 0.907898 | 0.7779881 | 0.8523917 | 0.9476356 | 0.7659878 | 0.9946709 |
| Phase-1 RCT-50 | 1.0071452 | 0.9869505 | 1.242203 | 1.1737882 | 0.9906365 | 1.1853511 | 1.1310492 |
| Sarcoplasmic reticulum calcium ATPase | 0.9791979 | 0.639021 | 0.848146 | 0.6005902 | 0.5034371 | 0.7016926 | 0.6593589 |
| Phase-1 RCT-207 | 0.9057024 | 0.7792742 | 0.703091 | 0.5536179 | 0.926182 | 0.9477801 | 0.9220814 |
| Acetyl-CoA carboxylase | 0.9278464 | 0.9941245 | 1.3206025 | 1.4031831 | 1.014609 | 1.0395695 | 0.9393954 |
| Cathepsin L, sequence 2 | 1.9677271 | 1.5778209 | 1.1545951 | 1.6601537 | 2.8350549 | 2.757117 | 2.138561 |
| Glucokinase | 1.0330616 | 0.6101243 | 0.6893839 | 0.5718501 | 0.5283005 | 0.7491081 | 0.6543379 |
| Gadd45 | 1.0598949 | 1.0560398 | 1.3325402 | 1.0159873 | 1.2725604 | 1.594505 | 1.0792844 |
| Phase-1 RCT-18 | 0.9587444 | 0.8838861 | 0.6834971 | 0.8107092 | 0.9253744 | 0.9976154 | 0.899071 |
| 3-beta-hydroxysteroid dehydrogenase | 0.8777147 | 1.0280991 | 1.1222498 | 1.1331949 | 1.4706546 | 1.7941374 | 1.2740374 |
| (HSD3B1) | | | | | | | |
| Phase-1 RCT-221 | 0.8979125 | 1.1710842 | 1.0370238 | 1.098748 | 1.0920931 | 1.0833702 | 1.0755467 |
| Heme oxygenase. | 1.0043232 | 1.373368 | 1.0865577 | 2.917187 | 1.071746 | 1.4373344 | 1.0967287 |
| Cyclin dependent kinase 4 | 0.8525141 | 0.879023 | 0.9254603 | 0.9883675 | 1.0628324 | 1.1004221 | 1.1061288 |
| Phase-1 RCT-75 | 0.8602893 | 1.0412214 | 0.8812035 | 0.8645427 | 1.0479089 | 0.9401539 | 1.1250124 |
| Phase-1 RCT-65 | 0.9521828 | 0.8271786 | 0.8493415 | 0.9684985 | 0.7222467 | 0.853315 | 0.8874497 |
| Cytochrome P450 1A1 | 1.0917714 | 0.8867841 | 1.0303569 | 0.9545401 | 0.7735831 | 1.0740471 | 1.0111408 |
| Peroxisomal multifunctional enzyme type II | 0.9851901 | 1.3593146 | 1.3733801 | 1.417047 | 1.5145717 | 1.1423458 | 1.1675475 |
| DNA topoisomerase I | 0.9236388 | 1.1730897 | 1.1526027 | 1.6801039 | 0.7845445 | 0.6841924 | 0.8817445 |
| Focal adhesion kinase (pp125FAK) | 1.0608697 | 0.8707502 | 0.9764838 | 0.9077843 | 0.8010594 | 0.9573495 | 1.0295125 |
| NGF-inducible anti-proliferative putative | 0.9104847 | 0.7925483 | 1.2518446 | 0.8924394 | 1.0145383 | 0.9233044 | 0.8045892 |
| secreted protein (PC3) | | | | | | | |
| Phase-1 RCT-82 | 0.9953193 | 0.9239647 | 0.8582541 | 0.8482337 | 0.8161439 | 1.0258021 | 0.9305265 |
| Senescence marker protein-30 | 0.4226844 | 1.0850809 | 0.759685 | 0.8979901 | 0.4350511 | 0.5446394 | 0.4577479 |
| Melanoma-associated antigen ME491 | 1.0640042 | 1.163345 | 1.1329628 | 1.0696158 | 1.0715066 | 1.1808499 | 1.0544521 |
| Activating transcription factor 3 | 1.0084718 | 0.8935916 | 0.888353 | 0.8893504 | 0.9308049 | 0.6867448 | 1.0154415 |
| Tryptophan hydroxylase | 0.6988825 | 1.1066045 | 1.335579 | 1.0396726 | 1.0393841 | 0.9811491 | 0.9435092 |
| Zinc finger protein | 1.0422287 | 0.8443515 | 0.7016313 | 0.6037316 | 0.8245552 | 0.8060321 | 0.9151603 |
| c-myc | 1.0701948 | 0.62925 | 0.6491546 | 0.5004997 | 0.533056 | 0.7381671 | 0.7143131 |
| Cholesterol 7-alpha-hydroxylase P450 | 0.7621476 | 0.8192132 | 0.6519031 | 0.6800177 | 0.5105014 | 0.6832889 | 0.7677417 |
| VII) | | | | | | | |
| Ferritin H-chain | 1.1021696 | 1.266814 | 1.6011648 | 1.3525403 | 1.2644123 | 1.1993645 | 1.0806979 |
| Phase-1 RCT-197 | 1.1441749 | 0.9955719 | 1.2469375 | 1.3207586 | 1.0216105 | 0.8816564 | 0.8798927 |
| Phase-1 RCT-179 | 0.9572094 | 1.2193669 | 0.975552 | 0.8569484 | 1.5513654 | 1.5742469 | 1.3420126 |
| Phase-1 RCT-15 | 0.9432314 | 1.1345271 | 1.3534527 | 1.3062931 | 1.1275568 | 1.2129607 | 1.1017004 |
| Argininosuccinate lyase | 1.2071607 | 1.1946032 | 1.6347252 | 1.2926433 | 1.7954997 | 1.6071215 | 1.2507344 |
| Phase-1 RCT-49 | 1.0139242 | 1.2256005 | 1.1066649 | 1.2001579 | 1.1663035 | 1.1443734 | 1.1782912 |
| Carbonic anhydrase III | 0.2394506 | 1.1618223 | 0.505354 | 0.6008899 | 0.3055729 | 0.3289917 | 0.3229253 |
| Proliferating cell nuclear antigen gene | 1.0515897 | 0.634904 | 0.6776711 | 0.6082113 | 0.7917095 | 1.0381975 | 0.9539039 |
| Integrin beta1 | 0.9279639 | 0.7730939 | 0.8124881 | 0.7983096 | 1.0477253 | 1.1413854 | 1.1250079 |
| Caspase 6 | 0.9580385 | 0.788857 | 0.806992 | 0.7584565 | 1.019783 | 1.242393 | 1.2042724 |
| Phase-1 RCT-116 | 0.8875603 | 1.394354 | 1.419673 | 1.0721742 | 1.4264121 | 1.1375471 | 0.9226086 |
| Phase-1 RCT-109 | 0.8845661 | 1.532065 | 1.803887 | 1.3284392 | 1.7225769 | 1.518066 | 1.3384665 |
| Phase-1 RCT-71 | 0.9996036 | 0.9384204 | 0.9716869 | 0.9555304 | 0.8514019 | 0.9863825 | 0.9400895 |
| Elongation factor-1 alpha | 0.9911292 | 1.3733981 | 1.4450591 | 1.7082874 | 1.452989 | 1.1024799 | 1.1718713 |
| Phase-1 RCT-169 | 1.2680169 | 0.6967915 | 0.720013 | 0.6509357 | 0.7454978 | 0.8677657 | 0.8084917 |
| Pyruvate kinase, muscle | 1.1942214 | 1.6536213 | 1.3679624 | 1.636046 | 2.1904655 | 2.153786 | 2.1795251 |
| Nucleosome assembly protein | 1.052492 | 1.9355768 | 1.2474529 | 2.1000085 | 1.2099686 | 1.8127214 | 1.4084079 |
| Phase-1 RCT-127 | 1.0212334 | 1.0907197 | 1.0291411 | 1.0535955 | 0.8133273 | 0.8068764 | 1.011674 |
| Iron-responsive element-binding protein | 1.1082592 | 1.0539323 | 1.05317 | 1.1206524 | 1.0602475 | 1.0169963 | 1.1373884 |
| Phase-1 RCT-72 | 0.9106965 | 0.8237481 | 0.8081779 | 0.8022819 | 0.8358537 | 1.0082793 | 0.9292724 |
| Phase-1 RCT-144 | 0.8136091 | 1.0663658 | 0.7956528 | 0.9034071 | 1.3248376 | 1.3309369 | 1.2203514 |
| Phase-1 RCT-242 | 1.0166258 | 1.056131 | 1.091847 | 0.9930441 | 0.9440522 | 1.0720447 | 1.0424666 |
| 17-beta hydroxysteroid dehydrogenase, | 1.0475473 | 2.3882864 | 1.3749642 | 1.9946938 | 1.6440188 | 2.8838308 | 1.6008005 |
| type 2 | | | | | | | |
| Phase-1 RCT-70 | 0.9146408 | 1.1891463 | 1.0299104 | 1.0342717 | 0.9232509 | 0.9500674 | 0.9728782 |
| Ribosomal protein L13A | 0.8737587 | 1.7495188 | 2.333353 | 1.8734444 | 2.3639362 | 1.7394975 | 1.6080854 |
| Cytochrome P450 2E1 | 1.0469469 | 0.8985198 | 0.8368296 | 0.6775463 | 0.8046962 | 1.3338028 | 1.0658755 |
| AT-3 | 1.0065508 | 1.1627029 | 1.1913149 | 1.1734469 | 1.0966538 | 1.0544688 | 1.0678574 |
| Phase-1 RCT-270 | 0.836407 | 0.9493927 | 0.8756394 | 1.0178083 | 0.7833051 | 0.8309505 | 0.8742222 |
| Phase-1 RCT-123 | 1.0217979 | 1.0832218 | 1.1344934 | 1.1581599 | 1.0141597 | 1.0704919 | 1.0927573 |
| Alpha-2-macroglobulin, sequence 2 | 1.1946173 | 0.9717218 | 0.7623529 | 1.0441406 | 0.9278002 | 1.0776229 | 0.9524019 |
| Matrix metalloproteinase-1 | 1.1472094 | 1.1123015 | 1.3156707 | 1.1344688 | 1.282249 | 1.2768875 | 1.1856162 |
| Beta-tubulin, class I | 0.6845306 | 1.3149364 | 1.4466263 | 1.298334 | 1.5714284 | 1.2117792 | 1.12632 |
| Phase-1 RCT-161 | 0.6867698 | 0.8951613 | 1.0139214 | 0.9247528 | 0.8414747 | 1.0606225 | 0.9530185 |
| 14-3-3 zeta | 0.8192508 | 1.1501281 | 0.6399084 | 0.7156639 | 1.3123472 | 5.873885 | 0.9658797 |
| Stem cell factor | 0.5359781 | 0.9755854 | 0.9229366 | 1.00816 | 0.5194036 | 0.6867824 | 0.5484479 |
| Macrophage inflammatory protein-2 alpha | 1.4132477 | 0.5719115 | 0.6779672 | 0.5192273 | 0.554804 | 2.1508055 | 1.0450137 |
| Thymidylate synthase | 1.119215 | 0.6350414 | 0.7131558 | 0.5718552 | 0.6282539 | 0.7601805 | 0.7745618 |
| c-jun | 1.126949 | 0.6171758 | 0.6665443 | 0.542862 | 0.6547394 | 0.8615619 | 0.7570067 |
| Superoxide dismutase Mn | 1.4382659 | 0.9251294 | 1.3578448 | 1.096616 | 1.5899603 | 1.2890095 | 1.063506 |
| Phase-1 RCT-73 | 0.9739215 | 1.1756834 | 1.4134628 | 1.3814272 | 1.225917 | 1.0566547 | 0.9840857 |
| Macrophage inflammatory protein-1 alpha | 0.7711114 | 0.7265784 | 0.7681252 | 0.6262415 | 0.8272222 | 0.7884836 | 0.707767 |
| Phase-1 RCT-214 | 0.8693917 | 0.9687756 | 0.9186413 | 1.0431243 | 0.9486495 | 0.9410788 | 0.9156182 |
| NADH-cytochrome b5 reductase | 1.1023754 | 0.7839085 | 0.856091 | 0.8732762 | 0.7179046 | 0.8234981 | 0.836085 |
| Interleukin-1 beta | 1.2392551 | 1.090416 | 1.2391316 | 1.1766851 | 1.2994987 | 1.2920426 | 1.168491 |
| NADP-dependent isocitrate | 0.865129 | 1.1322789 | 1.132729 | 1.0933871 | 1.209089 | 1.1112541 | 1.0987419 |
| dehydrogenase, cytosolic | | | | | | | |
| Phase-1 RCT-289 | 1.0496366 | 0.9434148 | 0.8331885 | 0.9777966 | 0.9990731 | 0.9006771 | 0.8395912 |
| Glutamine synthetase | 1.1833715 | 1.2668344 | 1.3885561 | 1.2699624 | 1.1902468 | 1.1897073 | 1.1165926 |
| Phase-1 RCT-182 | 1.1999097 | 1.0811398 | 1.2679977 | 1.2688823 | 1.0042474 | 1.1507543 | 1.0867915 |
| Ubiquitin conjugating enzyme (RAD 6 | 0.9750081 | 0.1630497 | 0.7036651 | 1.1223367 | 1.3116329 | 1.24775 | 1.1625859 |
| homologue) | | | | | | | |
| Extracellular-signal-regulated kinase 1 | 0.3300311 | 0.1252848 | 0.5081632 | 0.6125312 | 0.3046573 | 0.3350371 | 0.3205615 |
| Emerin | 0.9432057 | 0.2324685 | 0.9757874 | 0.9847849 | 1.0753324 | 0.8808404 | 1.0989051 |
| Phase-1 RCT-78 | 0.9232631 | 0.8948145 | 1.1908568 | 1.050119 | 1.0077131 | 1.0449177 | 1.0044048 |
| Phase-1 RCT-212 | 0.9089932 | 0.9186299 | 0.9894123 | 0.8484052 | 1.0486726 | 0.9406251 | 0.8525462 |
| Phase-1 RCT-168 | 1.0997419 | 1.4144046 | 0.914677 | 1.3156062 | 1.3835254 | 1.2019238 | 1.4046267 |
| Lysyl hydroxylase | 1.0035286 | 1.231724 | 1.5259194 | 1.3949589 | 1.2215214 | 1.2322268 | 1.1730435 |
| Phase-1 RCT-55 | 0.9243829 | 0.9101275 | 0.8160577 | 0.9212149 | 1.0833316 | 0.9998484 | 0.9518844 |
| MHC class I antigen RT1.A1(f) alpha- | 0.8926435 | 1.0682273 | 1.6476047 | 1.2695124 | 1.6141967 | 1.2436677 | 1.0448114 |
| chain | | | | | | | |
| Phase-1 RCT-40 | 0.921732 | 0.9916234 | 1.097505 | 1.2539204 | 0.8926131 | 0.8909886 | 0.8456137 |
| Cyclin G | 1.0974002 | 0.7445958 | 0.7131558 | 0.9847849 | 1.6917267 | 2.2594092 | 2.0401466 |
| Peroxisome proliferator activated receptor | 1.2582737 | 0.7031571 | 0.6796428 | 0.6014168 | 0.5681214 | 0.7162839 | 0.6415271 |
| gamma | | | | | | | |
| Phase-1 RCT-280 | 1.1028528 | 0.9702324 | 1.0636455 | 1.1562618 | 0.9759067 | 0.9970338 | 0.8945849 |
| D-dopachrome tautomerase | 0.8538515 | 1.2568195 | 1.3506292 | 1.2820061 | 1.3081354 | 1.3578203 | 1.1388144 |
| Phase-1 RCT-287 | 0.7506948 | 1.2428519 | 1.2898679 | 1.4175856 | 1.1620263 | 1.0603054 | 1.1059291 |
| Phase-1 RCT-119 | 1.2786301 | 0.6911626 | 0.6971992 | 0.8895224 | 0.4827038 | 0.5414503 | 0.6933811 |
| Thioredoxin-1 (Trx1) | 1.0454142 | 1.1316029 | 1.6981606 | 1.3663913 | 1.6787603 | 1.3549397 | 1.1133468 |
| Phase-1 RCT-225 | 0.5777342 | 0.749574 | 0.7150748 | 0.5892281 | 0.9577615 | 0.5963196 | 0.5246987 |
| Protein kinase C alpha | 1.0536807 | 0.7258531 | 0.8633379 | 0.8054474 | 1.0566401 | 1.0284588 | 0.9680486 |
| Phase-1 RCT-87 | 0.995598 | 0.9266088 | 0.9199709 | 0.9406554 | 0.9398181 | 0.9978488 | 0.9801689 |
| Phase-1 RCT-59 | 1.0034807 | 1.1143671 | 1.1807536 | 1.1362777 | 1.2706876 | 1.1089526 | 1.0547501 |
| C4b-binding protein | 0.9715494 | 1.2728775 | 1.2742926 | 1.4233519 | 1.0327133 | 1 | 0.9807597 |
| Phase-1 RCT-204 | 0.9405748 | 1.0662283 | 1.1155046 | 1.1442014 | 1.1805742 | 1.1189028 | 1.0334567 |
| Carnitine palmitoyl-CoA transferase | 1.0819365 | 0.8728707 | 0.8511443 | 0.715627 | 0.8950231 | 0.8905714 | 0.7958476 |
| 8-oxoguanine DNA glycosylase | 1.0741043 | 0.7874155 | 0.6968551 | 0.6841674 | 0.8569114 | 0.9105654 | 0.9317675 |
| H-rev107 | 0.9691979 | 1.2037599 | 1.2115896 | 1.2787749 | 1.0968711 | 1.2116033 | 1.1415013 |
| Preproalbumin | 0.9330527 | 1.0647217 | 1.2218138 | 1.2782254 | 1.0612347 | 0.9522716 | 0.816297 |
| Phase-1 RCT-177 | 0.9881506 | 1.1217731 | 1.2864312 | 1.3596256 | 0.97997 | 0.8885997 | 0.8830209 |
| Phase-1 RCT-110 | 0.9577065 | 1.154175 | 1.4480902 | 1.3403282 | 1.1795139 | 1.1601756 | 1.0831681 |
| Phase-1 RCT-227 | 0.899905 | 0.9692972 | 1.5969019 | 1.2403646 | 1.2766004 | 1.1081322 | 1.0679438 |
| Neuropeptide Y | 0.9556312 | 1.0106924 | 1.2744149 | 1.1949646 | 1.0286623 | 1.2185447 | 1.1740392 |
| Phase-1 RCT-277 | 1.205961 | 1.2383226 | 0.9787265 | 1.5964065 | 1.5029185 | 1.328338 | 1.6220429 |
| Phase-1 RCT-229 | 0.9487422 | 0.8706874 | 1.0495753 | 0.982382 | 0.8575376 | 1.0448686 | 1.0001718 |
| Phase-1 RCT-34 | 1.1890522 | 1.1823788 | 1.5438939 | 1.453539 | 1.3084046 | 1.2930677 | 1.250286 |
| Choline kinase | 0.7710573 | 0.7228806 | 0.8696287 | 0.6624301 | 0.5522103 | 0.6677852 | 0.6308965 |
| Phase-1 RCT-20 | 1.0165304 | 0.9496607 | 1.1574316 | 1.1306448 | 0.9617003 | 1.0999641 | 1.048287 |
| Phase-1 RCT-248 | 0.9628636 | 1.1949321 | 1.2771612 | 1.2190549 | 1.2454714 | 1.1927415 | 1.1324235 |
| | QUIN | QUIN | QUIN | QUIN | QUIN | QUIN | STRZ |
| Compound-Dose (2) | 25 | 25 | 25 | 100 | 100 | 100 | 20 |
| Animal Number (3) | 2541 | 2542 | 2543 | 2551 | 2552 | 2553 | 1721 |
| Liver Toxicity Necrosis Classification (4) | no | no | no | no | no | no | no |
| Gene Name (5) | | | | | | | |
| Gadd153 | 0.8956992 | 0.9289222 | 1.1091291 | 0.9515839 | 1.0174003 | 0.8959367 | 0.9536188 |
| Interferon related developmental regulator | 1.031074 | 0.9722404 | 1.1770204 | 1.024626 | 1.0792588 | 0.9846691 | 0.7433153 |
| IFRD1 (PC4) | | | | | | | |
| ID-1 | 1.0331286 | 1.082014 | 1.0738368 | 0.9660215 | 1.0264213 | 1.0321697 | 1.3289592 |
| Insulin-like growth factor binding protein 1 | 1.1078073 | 0.9879481 | 1.5970607 | 0.9672246 | 0.88195 | 0.739169 | 1.0312055 |
| NIPK | 1.2242166 | 1.065608 | 1.1189375 | 1.0729123 | 1.1398034 | 0.9694779 | 1.2761025 |
| Phase-1 RCT-50 | 1.0636328 | 1.0069976 | 1.2742422 | 1.1717432 | 1.0444403 | 1.0493222 | 1.0014558 |
| Sarcoplasmic reticulum calcium ATPase | 0.8214938 | 0.8958171 | 1.0185363 | 0.7674764 | 0.9952355 | 0.8174768 | 0.7976661 |
| Phase-1 RCT-207 | 1.1365346 | 1.1388465 | 2.2109659 | 1.2883872 | 1.136509 | 1.2409686 | 1.1293254 |
| Acetyl-CoA carboxylase | 0.9765118 | 0.8534338 | 1.1679797 | 0.992174 | 1.0005964 | 0.9210061 | 1.0434241 |
| Cathepsin L, sequence 2 | 1.0157638 | 1.0313617 | 1.8769704 | 1.0920159 | 1.0716906 | 1.2215364 | 1.0972161 |
| Glucokinase | 0.6419679 | 0.7991465 | 0.4694925 | 0.7633718 | 0.3221989 | 0.2473983 | 0.6508862 |
| Gadd45 | 0.93687 | 1.2487764 | 2.2416198 | 1.4244715 | 1.3627323 | 1.2921852 | 0.7954641 |
| Phase-1 RCT-18 | 0.954469 | 1.0060587 | 1.0009229 | 0.9983728 | 0.957477 | 0.929618 | 0.9614309 |
| 3-beta-hydroxysteroid dehydrogenase | 0.9914078 | 1.1018163 | 0.9779786 | 1.027558 | 0.8914278 | 0.8375758 | 0.9415546 |
| (HSD3B1) | | | | | | | |
| Phase-1 RCT-221 | 0.9750239 | 1.103459 | 1.1249943 | 1.0196245 | 0.9834673 | 0.9980653 | 1.1305263 |
| Heme oxygenase | 0.9515382 | 0.8371817 | 3.0129354 | 0.7271019 | 0.9996794 | 0.7949051 | 1.2329857 |
| Cyclin dependent kinase 4 | 0.9188529 | 1.2356969 | 0.9028722 | 0.940932 | 1.0109477 | 0.9624651 | 1.099637 |
| Phase-1 RCT-75 | 1.4734133 | 1.0174372 | 1.9605625 | 1.3797202 | 1.2384981 | 1.1150541 | 1.3240062 |
| Phase-1 RCT-65 | 1.0984735 | 1.0161941 | 0.7962401 | 1.1160252 | 1.0325493 | 0.9042594 | 1.840235 |
| Cytochrome P450 1A1 | 1.0424771 | 0.9274654 | 1.2526407 | 1.1283768 | 1.0869833 | 1.0470572 | 0.7936738 |
| Peroxisomal multifunctional enzyme type II | 1.0787268 | 1.0505832 | 0.8842874 | 0.9230731 | 0.9422285 | 0.9549543 | 1.2000835 |
| DNA topoisomerase I | 0.7656518 | 0.9416397 | 0.8486767 | 0.9307935 | 1.0130855 | 0.938291 | 1.0344342 |
| Focal adhesion kinase (pp125FAK) | 0.9549242 | 0.9735684 | 1.5041813 | 1.0836233 | 1.0837781 | 1.049512 | 0.8960605 |
| NGF-inducible anti-proliferative putative | 0.8378341 | 0.786496 | 0.7538254 | 0.7000711 | 0.885276 | 0.8201869 | 1.0825168 |
| secreted protein (PC3) | | | | | | | |
| Phase-1 RCT-82 | 1.0115731 | 0.9664413 | 0.8763434 | 0.9581664 | 0.9990774 | 0.9660887 | 1.0203491 |
| Senescence marker protein-30 | 0.811003 | 1.0388726 | 0.3998786 | 0.6833155 | 0.6631059 | 0.6771836 | 1.0503516 |
| Melanoma-associated antigen ME491 | 0.9839147 | 0.9234387 | 1.1317861 | 1.0217636 | 1.0110197 | 1.073723 | 1.1817913 |
| Activating transcription factor 3 | 1.4913332 | 1.4384993 | 1.2527953 | 1.348173 | 1.3015845 | 1.220127 | 1.0323893 |
| Tryptophan hydroxylase | 0.9619646 | 0.980993 | 0.8467742 | 1.0015317 | 1.0524276 | 0.8965089 | 1.0378932 |
| Zinc finger protein | 1.1104406 | 1.0909024 | 1.4057719 | 1.1470853 | 1.1203113 | 1.0748428 | 0.9613103 |
| c-myc | 1.0717872 | 0.9410529 | 1.4565594 | 1.113026 | 1.1461422 | 1.7793967 | 0.7935607 |
| Cholesterol 7-alpha-hydroxylase (P450 | 1.0538087 | 1.1232399 | 1.0750283 | 1.8360767 | 1.0966481 | 1.0650349 | 0.7443296 |
| VII) | | | | | | | |
| Ferritin H-chain | 0.913985 | 0.7783886 | 0.7563036 | 0.8323475 | 0.7851379 | 0.8347074 | 1.0425884 |
| Phase-1 RCT-197 | 0.895591 | 0.9750854 | 1.3339888 | 0.8430979 | 0.9143968 | 1.0166352 | 1.4878863 |
| Phase-1 RCT-179 | 1.0085516 | 1.1871773 | 1.8282133 | 1.076124 | 1.3230917 | 1.1721107 | 1.0385113 |
| Phase-1 RCT-15 | 1.1079892 | 1.0837233 | 1.6120588 | 1.0929176 | 1.0746158 | 1.1567411 | 1.3743663 |
| Argininosuccinate lyase | 1.4631919 | 1.4137486 | 2.2581432 | 1.794231 | 1.632306 | 2.0624561 | 1.4597362 |
| Phase-1 RCT-49 | 1.007575 | 1.0354266 | 1.6194397 | 1.1451591 | 1.2206876 | 1.2807841 | 0.8391562 |
| Carbonic anhydrase III | 0.8667812 | 1.23965 | 0.2986639 | 0.5571008 | 0.2518956 | 0.3976953 | 0.9823853 |
| Proliferating cell nuclear antigen gene | 1.0279797 | 0.9237141 | 1.3858143 | 1.0113871 | 1.047991 | 1.0082754 | 0.8578507 |
| Integrin beta1 | 1.085354 | 1.0687553 | 1.8026065 | 1.0774226 | 1.0116153 | 0.9653761 | 1.0033077 |
| Caspase 6 | 0.9021747 | 1.0317882 | 1.178977 | 0.9176208 | 0.8808754 | 0.9471948 | 1.0356376 |
| Phase-1 RCT-116 | 0.9547042 | 0.8843122 | 0.9255285 | 0.9102327 | 0.8723544 | 0.9200084 | 2.5868313 |
| Phase-1 RCT-109 | 0.97271 | 1.0947076 | 1.2864263 | 1.0713001 | 1.1132206 | 1.1474179 | 0.9519684 |
| Phase-1 RCT-71 | 0.8877336 | 0.8090476 | 0.982504 | 1.0523517 | 0.951045 | 1.035148 | 1.1118972 |
| Elongation factor-1 alpha | 0.6694363 | 0.896957 | 1.1260277 | 0.7227552 | 0.8398997 | 0.8872455 | 1.1026789 |
| Phase-1 RCT-169 | 1.0461854 | 0.8992772 | 0.9173954 | 0.8910572 | 0.9186046 | 0.9764186 | 1.0509863 |
| Pyruvate kinase, muscle | 1.0025909 | 0.9731379 | 1.4452989 | 1.1065384 | 1.0488223 | 1.1726681 | 1.1208653 |
| Nucleosome assembly protein | 0.9026749 | 1.121321 | 0.5443574 | 0.5778071 | 0.6209802 | 1.3077387 | 1.0815134 |
| Phase-1 RCT-127 | 1.0669646 | 0.7665519 | 1.308295 | 1.2098949 | 1.0791302 | 1.1112558 | 0.9420408 |
| Iron-responsive element-binding protein | 1.0171851 | 1.0931163 | 1.6909759 | 1.1853861 | 1.0078838 | 1.096173 | 1.1183586 |
| Phase-1 RCT-72 | 1.0119543 | 0.9829904 | 0.4851012 | 1.0945181 | 0.6444894 | 0.674148 | 0.964085 |
| Phase-1 RCT-144 | 1.0003207 | 1.071675 | 1.529523 | 1.1527525 | 1.3151917 | 1.259122 | 0.8401791 |
| Phase-1 RCT-242 | 1.0685512 | 0.9257383 | 1.5785475 | 1.1038216 | 1.0927283 | 1.0863065 | 0.8956102 |
| 17-beta hydroxysteroid dehydrogenase, | 0.981532 | 1.1265334 | 0.3681477 | 0.4599655 | 0.5136915 | 1.5952554 | 0.9851646 |
| type 2 | | | | | | | |
| Phase-1 RCT-70 | 1.5410239 | 1.1553749 | 0.9656294 | 1.6141089 | 1.4706594 | 1.1785955 | 1.1221439 |
| Ribosomal protein L13A | 1.0798548 | 1.1182297 | 1.2794975 | 1.1625522 | 1.141447 | 1.1276727 | 0.9722093 |
| Cytochrome P450 2E1 | 0.9380593 | 1.0358602 | 0.8484237 | 1.105311 | 1.090721 | 1.0681098 | 0.6198993 |
| AT-3 | 0.8638243 | 0.9531363 | 0.9970716 | 0.9087637 | 1.5190817 | 0.9012937 | 0.9033825 |
| Phase-1 RCT-270 | 0.9629648 | 0.8798907 | 0.4541825 | 0.7490899 | 0.6902649 | 0.8708327 | 1.1947669 |
| Phase-1 RCT-123 | 1.0666302 | 1.0512145 | 1.13857 | 1.0838073 | 1.099916 | 1.102237 | 0.8787104 |
| Alpha-2-macroglobulin, sequence 2 | 0.8252758 | 0.8752596 | 1.2112572 | 0.9450463 | 0.8576626 | 0.8717048 | 0.92966 |
| Matrix metalloproteinase-1 | 0.8307637 | 0.7999087 | 0.7917618 | 0.7988643 | 0.7848874 | 0.9125649 | 1.2903296 |
| Beta-tubulin, class I | 1.276248 | 1.1943134 | 0.8710172 | 1.24872 | 0.9432882 | 0.8042755 | 1.5540262 |
| Phase-1 RCT-161 | 1.0479895 | 0.9789994 | 0.9154271 | 0.9102461 | 0.9258651 | 0.9834871 | 1.1017041 |
| 14-3-3 zeta | 1.1426983 | 1.1011965 | 0.9550694 | 1.2807937 | 1.1686954 | 1.0333027 | 1.4088997 |
| Stem cell factor | 0.8031972 | 1.0344145 | 0.5544267 | 0.7409197 | 0.6971939 | 0.7673368 | 1.0957198 |
| Macrophage inflammatory protein-2 alpha | 0.9289722 | 0.8820543 | 1.5718418 | 0.8571358 | 0.9831936 | 1.1350106 | 1.0051125 |
| Thymidylate synthase | 0.8919495 | 1.0591632 | 0.977337 | 0.9519038 | 1.1447592 | 1.040263 | 0.9603848 |
| c-jun | 0.9860422 | 0.9228222 | 1.2308091 | 1.1535759 | 1.0465816 | 1.2275763 | 1.2747381 |
| Superoxide dismutase Mn | 1.2098013 | 1.1039717 | 3.784806 | 1.2279646 | 1.1624682 | 1.0292767 | 1.2160558 |
| Phase-1 RCT-73 | 1.0291015 | 0.9650838 | 0.6452626 | 0.9801879 | 1.0076854 | 0.9669374 | 1.0728621 |
| Macrophage inflammatory protein-1 alpha | 1.0617851 | 1.0436962 | 1.1756082 | 1.0974263 | 1.1763867 | 1.0912257 | 1.0375134 |
| Phase-1 RCT-214 | 1.1363833 | 1.0364236 | 0.671721 | 0.9653657 | 1.0425295 | 1.0555546 | 1.2341691 |
| NADH-cytochrome b5 reductase | 1.0764203 | 0.8795861 | 0.6485919 | 0.9086751 | 0.8445975 | 0.8003032 | 1.0803512 |
| Interleukin-1 beta | 0.9075565 | 0.8628155 | 0.879374 | 0.8918362 | 0.8959801 | 1.000017 | 1.1206225 |
| NADP-dependent isocitrate | 1.0648009 | 1.1678219 | 0.6985109 | 0.9796804 | 0.9319506 | 0.8092628 | 0.9009602 |
| dehydrogenase, cytosolic | | | | | | | |
| Phase-1 RCT-289 | 1.0372615 | 1.039138 | 0.7344763 | 0.9835274 | 0.8322235 | 0.9290679 | 0.8805066 |
| Glutamine synthetase | 0.863605 | 0.9052671 | 0.8548583 | 0.9265546 | 0.7997657 | 1.9257684 | 1.0959939 |
| Phase-1 RCT-182 | 0.9210967 | 0.8550212 | 0.5618068 | 0.8634859 | 0.6326014 | 0.7566448 | 0.9424531 |
| Ubiquitin conjugating enzyme (RAD 6 | 0.9900309 | 1.095867 | 1.4557115 | 1.0684187 | 1.1089792 | 1.2329906 | 0.938791 |
| homologue) | | | | | | | |
| Extracellular-signal-regulated kinase 1 | 0.8177642 | 1.2191478 | 0.3520504 | 0.5852303 | 0.3375069 | 0.4745552 | 1.2784476 |
| Emerin | 1.1757071 | 1.1222569 | 0.7269309 | 1.025147 | 1.2775453 | 1.1314534 | 0.9924032 |
| Phase-1 RCT-78 | 0.7667822 | 0.9350212 | 0.7710151 | 0.7003664 | 0.7711629 | 0.8144989 | 1.1142771 |
| Phase-1 RCT-212 | 0.8273309 | 0.8552477 | 0.997309 | 0.8805636 | 0.9675283 | 0.9095514 | 0.9827941 |
| Phase-1 RCT-168 | 0.8186531 | 0.8805174 | 0.8071098 | 0.7732949 | 0.823652 | 0.8864734 | 1.0231972 |
| Lysyl hydroxylase | 0.8935273 | 0.9140413 | 0.9552765 | 0.8748782 | 0.8614495 | 1.4383897 | 1.1423059 |
| Phase-1 RCT-55 | 1.0677035 | 0.9235227 | 1.8468018 | 1.3593582 | 1.1758628 | 1.0204117 | 0.9707851 |
| MHC class I antigen RT1.A1(f) alpha- | 1.3959838 | 0.9441919 | 1.199634 | 1.4748625 | 1.2825601 | 1.1630158 | 2.5953853 |
| chain | | | | | | | |
| Phase-1 RCT-40 | 0.9116669 | 0.7892057 | 0.4999414 | 0.8213936 | 0.7035756 | 0.5676172 | 1.2832835 |
| Cyclin G | 1.0510112 | 1.0155321 | 1.515248 | 1.0035025 | 1.1046528 | 1.1842393 | 1.911375 |
| Peroxisome proliferator activated receptor | 2.198169 | 0.9721372 | 0.9115514 | 0.854784 | 1.1268333 | 1.0050257 | 0.8751646 |
| gamma | | | | | | | |
| Phase-1 RCT-280 | 1.0679547 | 1.0386404 | 0.789385 | 0.9683741 | 1.1255198 | 1.1483499 | 1.0559148 |
| D-dopachrome tautomerase | 0.7993314 | 0.8184301 | 0.4081545 | 0.8874857 | 0.5904214 | 0.5719094 | 1.1111488 |
| Phase-1 RCT-287 | 0.8157653 | 0.9213677 | 0.9328461 | 0.8108144 | 0.8417811 | 0.8704904 | 1.0598764 |
| Phase-1 RCT-119 | 1.0210013 | 0.9362307 | 0.9537554 | 0.9951758 | 0.9582984 | 0.9379306 | 0.6758112 |
| Thioredoxin-1 (Trx1) | 0.9269302 | 0.8248388 | 0.9475777 | 0.9083909 | 0.8447252 | 0.8506291 | 0.9776722 |
| Phase-1 RCT-225 | 0.8234596 | 0.8645654 | 1.3236755 | 0.6038162 | 0.8950652 | 0.6956808 | 2.0690262 |
| Protein kinase C alpha | 0.8521603 | 1.0295439 | 0.8930458 | 0.9142309 | 0.9208859 | 0.9357069 | 0.8531994 |
| Phase-1 RCT-87 | 0.9660085 | 0.8717729 | 0.8197535 | 0.9459957 | 0.9455392 | 0.87264 | 0.9880917 |
| Phase-1 RCT-59 | 1.063215 | 1.0678072 | 1.1595917 | 1.0817872 | 1.0348437 | 1.1109334 | 1.0723096 |
| C4b-binding protein | 0.8018779 | 0.8555282 | 1.045143 | 0.861671 | 1.2936996 | 0.9341326 | 0.8494085 |
| Phase-1 RCT-204 | 1.104904 | 1.0693393 | 1.0495746 | 1.2675121 | 1.1946778 | 1.241528 | 1.0332555 |
| Carnitine palmitoyl-CoA transferase | 1.1108785 | 1.2050971 | 1.5716841 | 1.09616 | 1.5232165 | 1.6343318 | 1.1482769 |
| 8-oxoguanine DNA glycosylase | 1.0348185 | 1.0606564 | 1.9593476 | 1.0424011 | 1.1352636 | 1.1337317 | 1.0243404 |
| H-rev107 | 1.0823803 | 1.006325 | 1.203018 | 1.1564531 | 1.1619513 | 1.0987496 | 0.9544323 |
| Preproalbumin | 1.4256947 | 1.0851679 | 0.9119346 | 0.9170877 | 0.9119481 | 1.0372233 | 1.2458792 |
| Phase-1 RCT-177 | 0.9769375 | 0.8831378 | 0.6110009 | 0.9279498 | 1.0051912 | 0.8675136 | 1.1267495 |
| Phase-1 RCT-110 | 0.9416219 | 0.9235636 | 0.9518443 | 1.0017676 | 0.9444591 | 0.9300977 | 0.9094245 |
| Phase-1 RCT-227 | 1.0005257 | 0.9662607 | 0.8116308 | 0.6630796 | 0.7478554 | 0.797029 | 0.9695783 |
| Neuropeptide Y | 1.0690722 | 0.9926771 | 1.0389087 | 1.1013191 | 0.9941952 | 0.955124 | 0.8459478 |
| Phase-1 RCT-277 | 1.0591295 | 1.0165619 | 0.5802853 | 0.9690122 | 1.3698503 | 0.9765036 | 0.9593526 |
| Phase-1 RCT-229 | 0.8854107 | 0.9401656 | 0.8767061 | 0.8490628 | 0.8596894 | 0.917158 | 0.8808166 |
| Phase-1 RCT-34 | 1.0768846 | 1.0713792 | 0.6672249 | 0.9171392 | 0.8265589 | 0.8609718 | 1.3573017 |
| Choline kinase | 0.9044166 | 0.8752269 | 1.042818 | 1.0192693 | 0.9233241 | 0.9211072 | 1.7457666 |
| Phase-1 RCT-20 | 1.0182931 | 1.0163316 | 1.1525867 | 0.9718862 | 1.0160118 | 1.0152878 | 1.0331981 |
| Phase-1 RCT-248 | 0.9878134 | 1.00088 | 1.2434682 | 1.0404116 | 1.0229217 | 1.0917748 | 0.9117446 |
| | STRZ | STRZ | STRZ | STRZ | STRZ | TAM | TAM |
| Compound-Dose (2) | 20 | 20 | 75 | 75 | 75 | 50 | 50 |
| Animal Number (3) | 1722 | 1723 | 1731 | 1732 | 1733 | 1441 | 1442 |
| Liver Toxicity Necrosis Classification (4) | no | no | no | no | no | no | no |
| Gene Name (5) | | | | | | | |
| Gadd153 | 1.0737689 | 1.1766455 | 0.9160689 | 0.9364167 | 0.9432371 | 1.0912793 | 1.5739099 |
| Interferon related developmental regulator | 0.8627251 | 0.7559729 | 0.8017629 | 0.9494509 | 0.9266053 | 1.0711445 | 0.9737964 |
| IFRD1 (PC4) | | | | | | | |
| ID-1 | 1.1216054 | 1.153467 | 1.8172499 | 1.3095882 | 1.1098682 | 1.0097964 | 1.27443 |
| Insulin-like growth factor binding protein 1 | 1.1805946 | 1.1066333 | 0.9161218 | 1.0492432 | 1.1368207 | 1.7345412 | 0.8906694 |
| NIPK | 0.9979778 | 1.2532598 | 1.2405387 | 1.2636437 | 1.1432594 | 1.2778863 | 1.2165554 |
| Phase-1 RCT-50 | 0.8421992 | 1.5487115 | 1.3775797 | 0.8643315 | 0.8075433 | 1.0551274 | 1.0340571 |
| Sarcoplasmic reticulum calcium ATPase | 1.0155424 | 0.8899127 | 0.8746518 | 0.8632819 | 0.8709957 | 1.1319916 | 1.4589427 |
| Phase-1 RCT-207 | 0.954851 | 1.4967248 | 3.1272452 | 1.0708675 | 0.8985648 | 1.0309031 | 1.202655 |
| Acetyl-CoA carboxylase | 0.8430436 | 0.803058 | 0.8433398 | 0.9405202 | 1.0811574 | 0.8993139 | 0.8327028 |
| Cathepsin L, sequence 2 | 1.4523062 | 1.2590424 | 1.658361 | 1.1082938 | 1.1572012 | 0.7108592 | 0.788115 |
| Glucokinase | 1.0789497 | 0.9115734 | 0.5019409 | 0.8870753 | 1.0028398 | 0.9253835 | 2.0241113 |
| Gadd45 | 0.7920815 | 1.2017158 | 0.8048852 | 0.7681376 | 0.9217603 | 1.8175162 | 2.3321905 |
| Phase-1 RCT-18 | 0.9095823 | 0.9857608 | 0.9504393 | 0.980422 | 1.0891905 | 1.0187443 | 0.8855822 |
| 3-beta-hydroxysteroid dehydrogenase | 0.8510614 | 0.9739038 | 0.793359 | 1.159552 | 0.7461191 | 1.2947807 | 1.0412668 |
| (HSD3B1) | | | | | | | |
| Phase-1 RCT-221 | 1.1687772 | 1.1242981 | 1.1561108 | 1.2108594 | 1.0200552 | 1.0277013 | 0.8851076 |
| Heme oxygenase | 0.8334703 | 1.0286058 | 0.8998796 | 1.1155692 | 1.1191038 | 1.4631745 | 1.5392202 |
| Cyclin dependent kinase 4 | 1.2424027 | 1.1119636 | 1.152386 | 0.965961 | 1.0467372 | 1.080344 | 0.900196 |
| Phase-1 RCT-75 | 1.2158002 | 1.231854 | 2.237655 | 1.1456001 | 1.058911 1 | 0.9709264 | 1.0833299 |
| Phase-1 RCT-65 | 1.39018 | 1.5763934 | 1.3295559 | 1.4511452 | 1.621822 | 1.164027 | 1.1208055 |
| Cytochrome P450 1A1 | 0.724393 | 0.8753935 | 0.6071625 | 0.6283435 | 0.6847531 | 0.9990615 | 1.026141 |
| Peroxisomal multifunctional enzyme type II | 1.2785238 | 1.2402827 | 1.0497079 | 1.700979 | 1.5461411 | 0.9999176 | 0.9293192 |
| DNA topoisomerase I | 1.0377209 | 0.9506957 | 0.9657524 | 1.3195816 | 1.1731496 | 1.137121 | 0.9085392 |
| Focal adhesion kinase (pp125FAK) | 0.8808481 | 0.8968087 | 0.972373 | 0.9002278 | 0.8614476 | 0.985641 | 0.9100221 |
| NGF-inducible anti-proliferative putative | 1.0194911 | 1.2836846 | 1.3566833 | 1.0795976 | 0.9261039 | 0.7677426 | 0.7673935 |
| secreted protein (PC3) | | | | | | | |
| Phase-1 RCT-82 | 0.9578578 | 0.9739674 | 0.9157137 | 0.8598458 | 0.9127147 | 0.9682602 | 0.8709383 |
| Senescence marker protein-30 | 1.278451 | 0.8411581 | 0.6140094 | 0.9527337 | 1.104355 | 0.8805426 | 0.9455206 |
| Melanoma-associated antigen ME491 | 1.0874984 | 1.0837638 | 0.960524 | 1.130692 | 0.9983096 | 0.9105845 | 1.1453857 |
| Activating transcription factor 3 | 0.8574825 | 1.2900977 | 0.7988586 | 0.7648174 | 0.7643014 | 1.1931349 | 1.2708017 |
| Tryptophan hydroxylase | 0.9964554 | 1.028839 | 1.0020759 | 1.0855097 | 1.1375448 | 0.8443174 | 0.7492142 |
| Zinc finger protein | 0.8400785 | 1.4572549 | 0.8137144 | 0.8584415 | 0.8962095 | 1.0151678 | 0.875479 |
| c-myc | 0.844269 | 1.0434324 | 0.9041742 | 0.8724206 | 0.7673276 | 1.2086236 | 1.6091957 |
| Cholesterol 7-alpha-hydroxylase (P450 | 0.8700164 | 0.8101485 | 0.9179509 | 0.889387 | 0.7751555 | 1.2687346 | 1.6810906 |
| VII) | | | | | | | |
| Ferritin H-chain | 1.4046493 | 0.9548562 | 1.3895175 | 1.1566914 | 1.2770388 | 0.7162639 | 0.6776546 |
| Phase-1 RCT-197 | 1.2378111 | 1.5079869 | 2.5526888 | 1.0287683 | 1.0929102 | 1.0487497 | 1.1231748 |
| Phase-1 RCT-179 | 1.075454 | 1.4120117 | 2.291223 | 1.2200727 | 1.0791081 | 0.9745325 | 0.8465593 |
| Phase-1 RCT-15 | 1.2449303 | 1.5794407 | 1.5696763 | 1.3020574 | 1.5605807 | 0.9679531 | 0.7425766 |
| Argininosuccinate lyase | 1.6605521 | 1.6711084 | 4.266252 | 1.8461033 | 1.825206 | 0.9949393 | 0.7586735 |
| Phase-1 RCT-49 | 0.9213126 | 1.1358856 | 2.317324 | 0.9700708 | 0.8558337 | 1.0637664 | 1.2273704 |
| Carbonic anhydrase III | 0.7580923 | 0.7746936 | 0.667691 | 1.200777 | 1.2550609 | 0.9223427 | 0.8787487 |
| Proliferating cell nuclear antigen gene | 0.8890767 | 0.9662833 | 1.0041722 | 0.8418013 | 0.7328724 | 1.0263379 | 1.9752729 |
| Integrin beta1 | 1.1413392 | 0.9294103 | 1.0556656 | 0.8190725 | 0.8903053 | 1.3158003 | 1.2657907 |
| Caspase 6 | 1.2187108 | 1.0561849 | 1.2821721 | 0.9734382 | 1.0541339 | 1.1723139 | 1.3769325 |
| Phase-1 RCT-116 | 1.5067525 | 1.6781765 | 1.4246353 | 1.8511719 | 1.7373319 | 0.9943922 | 0.9319395 |
| Phase-1 RCT-109 | 1.15137 | 1.0343615 | 1.5914884 | 1.1699181 | 1.0075294 | 1.0446274 | 0.9255508 |
| Phase-1 RCT-71 | 1.1302744 | 1.2786307 | 0.9923076 | 1.0060652 | 1.1564497 | 0.9760051 | 0.8613675 |
| Elongation factor-1 alpha | 1.2483238 | 1.0577475 | 1.248002 | 1.609456 | 1.1995648 | 0.8827937 | 0.7759746 |
| Phase-1 RCT-169 | 0.8830833 | 0.9073314 | 0.6237474 | 0.6729012 | 0.7920311 | 1.1518811 | 1.1359924 |
| Pyruvate kinase, muscle | 1.2165269 | 1.1340832 | 1.2497878 | 0.7278349 | 1.1058248 | 0.8467429 | 1.0629312 |
| Nucleosome assembly protein | 0.9386222 | 1.0385602 | 0.8845083 | 0.989705 | 1.1036022 | 0.8301776 | 1.2256218 |
| Phase-1 RCT-127 | 0.9229556 | 1.0349848 | 1.503476 | 1.0240353 | 0.9162713 | 1.0824753 | 1.2737762 |
| Iron-responsive element-binding protein | 1.2228448 | 1.0449297 | 0.9889963 | 1.2254508 | 1.3025224 | 0.9084047 | 1.0185213 |
| Phase-1 RCT-72 | 0.8637832 | 0.9814218 | 0.8941643 | 0.7566846 | 0.8560301 | 1.0658915 | 1.1128669 |
| Phase-1 RCT-144 | 1 | 1.232107 | 1.8422887 | 1.1571612 | 0.8360657 | 1.0359 | 1.1833651 |
| Phase-1 RCT-242 | 0.747821 | 1.3343729 | 1.5025367 | 0.9211109 | 0.7305669 | 1.3234891 | 1.7899241 |
| 17-beta hydroxysteroid dehydrogenase, | 1.141976 | 1.3813183 | 0.3960133 | 0.9441239 | 1.5162907 | 0.821476 | 1.2831832 |
| type 2 | | | | | | | |
| Phase-1 RCT-70 | 1.0750538 | 1.090064 | 1.0416764 | 1.0039694 | 0.8851922 | 0.9657338 | 0.8298022 |
| Ribosomal protein L13A | 1.2723142 | 1.2403827 | 1.82943 | 1.1495602 | 1.2551616 | 1.0172838 | 0.8620865 |
| Cytochrome P450 2E1 | 0.7333723 | 0.8871392 | 0.708752 | 0.685261 | 0.6611009 | 0.5719594 | 0.9444408 |
| AT-3 | 0.8373443 | 0.9056959 | 0.9913174 | 0.939988 | 0.8855007 | 0.9905794 | 0.9608445 |
| Phase-1 RCT-270 | 1.2054355 | 0.8441143 | 0.763102 | 1.2004589 | 1.6963117 | 0.9519514 | 0.9581122 |
| Phase-1 RCT-123 | 0.9229906 | 0.8862519 | 0.8503408 | 0.9936705 | 0.86371 | 0.9537707 | 0.8667439 |
| Alpha-2-macroglobulin, sequence 2 | 0.9479917 | 1.0496235 | 1.7544894 | 1.2053983 | 1.1606668 | 1.0779585 | 0.9964705 |
| Matrix metalloproteinase-1 | 1.4198823 | 1.0354248 | 1.5808303 | 1.2799987 | 1.3507673 | 0.8185946 | 0.7619056 |
| Beta-tubulin, class I | 1.293969 | 1.2116205 | 1.9491514 | 1.497107 | 1.0653915 | 0.9279938 | 0.8029044 |
| Phase-1 RCT-161 | 0.7404725 | 0.8566369 | 0.6832569 | 1.0638947 | 1.02087 | 0.8813285 | 0.8723533 |
| 14-3-3 zeta | 0.9558833 | 1.2764328 | 1.3789151 | 1.1966808 | 0.96464 | 1.0300121 | 1.0259553 |
| Stem cell factor | 1.196071 | 1.0580199 | 0.7439016 | 0.8890051 | 1.2871227 | 0.9191636 | 0.9517368 |
| Macrophage inflammatory protein-2 alpha | 0.9721848 | 1.6220434 | 0.9330153 | 0.8148239 | 0.8873224 | 2.3272166 | 1.7307833 |
| Thymidylate synthase | 1.0069455 | 0.9423463 | 0.8863912 | 0.8430626 | 0.8262504 | 0.8203632 | 1.0684782 |
| c-jun | 1.16242 | 2.3929214 | 1.1900374 | 1.4642601 | 1.8177665 | 1.286165 | 1.5384387 |
| Superoxide dismutase Mn | 1.2905015 | 1.3027042 | 1.2103195 | 1.2329135 | 1.4635897 | 0.991625 | 1.5268167 |
| Phase-1 RCT-73 | 1.0187111 | 1.0207121 | 0.8857792 | 1.1207213 | 0.9580408 | 0.967236 | 0.87017 |
| Macrophage inflammatory protein-1 alpha | 0.853749 | 1.021516 | 0.7008354 | 0.6948373 | 0.7732546 | 1.0142543 | 1.7530977 |
| Phase-1 RCT-214 | 0.8972353 | 1.0452962 | 0.6276279 | 0.915686 | 0.9189161 | 0.8379453 | 0.8361688 |
| NADH-cytochrome b5 reductase | 1.1936305 | 1.0679834 | 1.0838271 | 1.2406534 | 1.4356691 | 0.7777835 | 0.8633484 |
| Interleukin-1 beta | 1.2247337 | 0.980786 | 1.0955127 | 0.912331 | 1.0534362 | 0.788668 | 0.723652 |
| NADP-dependent isocitrate | 0.8780353 | 0.966067 | 0.9328791 | 0.9244144 | 0.8056212 | 0.8415794 | 0.7889218 |
| dehydrogenase, cytosolic | | | | | | | |
| Phase-1 RCT-289 | 0.8883271 | 0.9851575 | 0.9803476 | 1.0281278 | 0.9742168 | 0.8606963 | 0.7975212 |
| Glutamine synthetase | 1.4409302 | 1.3269619 | 1.1463455 | 1.3835598 | 1.4972576 | 0.9179347 | 1.302443 |
| Phase-1 RCT-182 | 1.0593579 | 0.9943004 | 0.8284189 | 1.0955104 | 1.2568598 | 0.9519564 | 0.9036076 |
| Ubiquitin conjugating enzyme (RAD 6 | 0.8755921 | 0.9556578 | 1.0303826 | 0.8045185 | 0.8296448 | 1.0137633 | 0.9944734 |
| homologue) | | | | | | | |
| Extracellular-signal-regulated kinase 1 | 0.9172936 | 1.096869 | 0.9084079 | 1.1666744 | 1.4342372 | 0.9839463 | 1.0247432 |
| Emerin | 0.9539074 | 1.1304243 | 1.2213633 | 1.1044062 | 1.0047426 | 0.9526151 | 1.0066795 |
| Phase-1 RCT-78 | 1.1278096 | 0.9347562 | 1.0484998 | 1.1015949 | 1.3498646 | 1.0371861 | 0.9282566 |
| Phase-1 RCT-212 | 0.9522099 | 1.0293903 | 1.0131068 | 0.8411049 | 0.9560429 | 0.9642072 | 1.1934193 |
| Phase-1 RCT-168 | 1.0624039 | 0.9763295 | 0.7699958 | 1.101075 | 1.1696348 | 0.8539112 | 0.7102844 |
| Lysyl hydroxylase | 1.1107872 | 1.191205 | 1.2542695 | 1.1558717 | 1.0565826 | 1.0550195 | 1.2478005 |
| Phase-1 RCT-55 | 0.9513087 | 0.9224072 | 0.713701 | 0.8143131 | 0.8748555 | 0.7872616 | 0.8144665 |
| MHC class I antigen RT1.A1(f) alpha- | 1.7178133 | 1.8646669 | 2.0844798 | 2.0641513 | 1.9905267 | 1.1914444 | 1.0610402 |
| chain | | | | | | | |
| Phase-1 RCT-40 | 0.9140392 | 0.9514493 | 0.7298446 | 1.1800665 | 1.2965161 | 0.8948373 | 0.798094 |
| Cyclin G | 1.448197 | 2.3561683 | 2.9436986 | 0.8907791 | 1.1202722 | 1.0352434 | 1.7809172 |
| Peroxisome proliferator activated receptor | 0.875436 | 1.0241729 | 0.6734084 | 0.7730314 | 0.8243588 | 0.7773308 | 0.8697829 |
| gamma | | | | | | | |
| Phase-1 RCT-280 | 0.9063671 | 1.171397 | 0.8746454 | 0.8242911 | 0.9050973 | 0.9328898 | 0.718339 |
| D-dopachrome tautomerase | 1.388199 | 1.0215019 | 1.4876618 | 1.395042 | 1.4402454 | 0.7842439 | 0.7951776 |
| Phase-1 RCT-287 | 1.0029128 | 0.9802625 | 0.9326011 | 1.2809837 | 1.2091691 | 0.8809412 | 0.9437203 |
| Phase-1 RCT-119 | 0.5912408 | 0.8081309 | 0.6781581 | 1.124735 | 2.5603762 | 1.2905766 | 1.0359875 |
| Thioredoxin-1 (Trx1) | 0.9818854 | 0.9173943 | 1.1061931 | 1.0020434 | 1.1464458 | 1.0211014 | 1.2433465 |
| Phase-1 RCT-225 | 1.0739199 | 1.6913116 | 1.5733751 | 1.0460331 | 0.8866406 | 0.9475759 | 0.981434 |
| Protein kinase C alpha | 0.9595003 | 0.9461255 | 0.9542564 | 0.8616415 | 0.8620582 | 0.9786876 | 1.1645181 |
| Phase-1 RCT-87 | 0.9916799 | 1.0079222 | 0.9778675 | 0.9168944 | 0.9378612 | 0.8750016 | 0.8380058 |
| Phase-1 RCT-59 | 0.8672726 | 1.5311468 | 3.6316793 | 0.8235968 | 0.7034488 | 0.9753933 | 0.8246062 |
| C4b-binding protein | 0.757324 | 0.8601477 | 0.7279925 | 1.2847617 | 1.2831093 | 1.1018332 | 0.9943827 |
| Phase-1 RCT-204 | 1.0999635 | 0.9623228 | 1.2868581 | 1.1265236 | 1.0920818 | 0.9673375 | 0.8572375 |
| Carnitine palmitoyl-CoA transferase | 0.9195423 | 1.4491287 | 1.2275239 | 1.0454147 | 0.8815506 | 0.9117487 | 1.2284262 |
| 8-oxoguanine DNA glycosylase | 0.9382048 | 0.9562076 | 0.8322278 | 0.7643086 | 0.8743201 | 1.0137247 | 1.340345 |
| H-rev107 | 0.9051517 | 0.8962622 | 0.9432707 | 0.8827562 | 0.8446095 | 0.997159 | 1.1055218 |
| Preproalbumin | 0.9610689 | 1.2981279 | 1.0301318 | 1.3040833 | 1.1630982 | 0.922419 | 0.6620168 |
| Phase-1 RCT-177 | 1.109714 | 1.0483027 | 0.9069247 | 1.2175887 | 1.2405002 | 0.9616411 | 0.8026573 |
| Phase-1 RCT-110 | 0.8324231 | 0.9369552 | 0.7869 | 0.8053718 | 0.7728071 | 1.1483692 | 1.6965662 |
| Phase-1 RCT-227 | 1.2347263 | 0.9367322 | 1.1006217 | 1.5316616 | 1.5598246 | 1.0327357 | 0.9355939 |
| Neuropeptide Y | 0.8240988 | 0.9039084 | 0.8648127 | 0.7982968 | 0.8984295 | 1.0719379 | 0.9919192 |
| Phase-1 RCT-277 | 1.0315834 | 1.2350388 | 1.1656072 | 1.0456783 | 0.9077155 | 0.5746481 | 0.4152244 |
| Phase-1 RCT-229 | 0.8551132 | 0.8686208 | 0.7659316 | 0.8087411 | 0.8570358 | 1.0255709 | 1.0013528 |
| Phase-1 RCT-34 | 1.1473901 | 0.9467121 | 1.1969974 | 1.292775 | 1.1435173 | 0.8790201 | 1.1033767 |
| Choline kinase | 1.0691828 | 1.2637377 | 0.9556332 | 0.682479 | 1.0301757 | 1.0825635 | 1.222488 |
| Phase-1 RCT-20 | 1.0060068 | 1.0682278 | 1.036447 | 0.9440693 | 1.1107574 | 1.1353658 | 1.2166934 |
| Phase-1 RCT-248 | 0.9038248 | 0.9292738 | 0.9618236 | 0.8613879 | 0.7903947 | 1.0160334 | 1.0504133 |

| | TAM | TAM | TAM | TAM | TET | TET |
|---|---|---|---|---|---|---|
| Compound-Dose (2) | 50 | 200 | 200 | 200 | 50 | 50 |
| Animal Number (3) | 1443 | 1451 | 1452 | 1453 | 121 | 122 |
| Liver Toxicity Necrosis Classification (4) | no | no | no | no | no | no |
| Gene Name (5) | | | | | | |
| Gadd153 | 1.0152202 | 1.100836 | 1.263481 | 1.4057178 | 1.2102058 | 1.3193202 |
| Interferon related developmental regulator | 1.0953354 | 1.2916914 | 1.5498939 | 1.7393152 | 1.0691351 | 1.0795922 |
| IFRD1 (PC4) | | | | | | |
| ID-1 | 0.8682674 | 0.9226274 | 0.9420359 | 0.991776 | 1.0916549 | 1.284487 |
| Insulin-like growth factor binding protein 1 | 1.0075512 | 1.1390456 | 1.3517617 | 1.7847229 | 1.0978009 | 2.230742 |
| NIPK | 1.1002748 | 0.9807164 | 1.104517 | 1.2421268 | 1.1071142 | 2.668978 |
| Phase-1 RCT-50 | 1.124537 | 1.1887858 | 1.2070966 | 1.1305026 | 1.1067357 | 1.2325734 |
| Sarcoplasmic reticulum calcium ATPase | 1.2333715 | 1.1359707 | 1.2044669 | 1.136322 | 1.1692326 | 1.0805093 |
| Phase-1 RCT-207 | 0.9959706 | 1.1011415 | 1.1091803 | 1.0794061 | 0.9655142 | 0.9223894 |
| Acetyl-CoA carboxylase | 0.8801596 | 1.6661114 | 1.1750461 | 1.5631254 | 0.9902887 | 1.297844 |
| Cathepsin L, sequence 2 | 0.6989568 | 0.7726345 | 0.9096466 | 1.0990357 | 1.1176881 | 1.0854284 |
| Glucokinase | 0.7165741 | 1.2968189 | 0.9717113 | 0.4741327 | 0.9263198 | 1.0452824 |
| Gadd45 | 2.429594 | 1.3961899 | 1.9527471 | 1.752101 | 1.1292982 | 1.3658636 |
| Phase-1 RCT-18 | 0.9971563 | 0.9556968 | 1.004604 | 0.9604754 | 0.9914231 | 1.0088214 |
| 3-beta-hydroxysteroid dehydrogenase | 1.4430685 | 1.4229106 | 1.2230406 | 1.1908451 | 0.8068038 | 0.8191238 |
| (HSD3B1) | | | | | | |
| Phase-1 RCT-221 | 0.9591357 | 1.0952033 | 1.2028964 | 1.0389504 | 0.8833555 | 1.1491623 |
| Heme oxygenase | 1.1466633 | 2.1472974 | 4.2234936 | 3.2589738 | 1.0094517 | 1.46658 |
| Cyclin dependent kinase 4 | 0.8506956 | 0.8447105 | 0.9750285 | 1.0314125 | 1.0638285 | 1.2540737 |
| Phase-1 RCT-75 | 0.9578995 | 1.072364 | 1.0616248 | 1.1798095 | 0.9794541 | 1.3388108 |
| Phase-1 RCT-65 | 1.0241032 | 1.0527843 | 1.0345284 | 1.0379571 | 0.8423903 | 0.870319 |
| Cytochrome P450 1A1 | 0.8676978 | 0.8672437 | 0.9412964 | 0.9138129 | 1.0405208 | 1.1254727 |
| Peroxisomal multifunctional enzyme type II | 1.0388504 | 1.1712062 | 1.1154138 | 1.0727375 | 0.8296418 | 1.0383184 |
| DNA topoisomerase I | 1.0252452 | 1.3391616 | 1.4383702 | 1.5401082 | 0.983231 | 1.2396675 |
| Focal adhesion kinase (pp125FAK) | 0.8517487 | 1.2039348 | 1.4319284 | 1.5430435 | 1.0872172 | 1.1564238 |
| NGF-inducible anti-proliferative putative | 0.7386558 | 0.786059 | 0.7738565 | 0.8695411 | 0.9580157 | 1.1197777 |
| secreted protein (PC3) | | | | | | |
| Phase-1 RCT-82 | 0.8864622 | 0.8628906 | 0.9813061 | 0.8432906 | 0.9942754 | 0.9654738 |
| Senescence marker protein-30 | 1.1650062 | 0.9105342 | 0.8735455 | 0.589164 | 1.0935825 | 0.8975534 |
| Melanoma-associated antigen ME491 | 1.003725 | 0.8521228 | 0.9489439 | 1.1092868 | 2.466284 | 1.1839517 |
| Activating transcription factor 3 | 1.3001816 | 1.1631835 | 0.9209164 | 1.0283355 | 1.0802057 | 0.8402438 |
| Tryptophan hydroxylase | 0.9135286 | 1.1381304 | 0.8054601 | 0.9772335 | 0.9127503 | 0.9082804 |
| Zinc finger protein | 1.0066345 | 1.2082301 | 1.4653074 | 1.0416542 | 0.9855456 | 1.1094133 |
| c-myc | 1.2398218 | 1.2368919 | 1.9127817 | 1.4010482 | 1.0826228 | 1.249958 |
| Cholesterol 7-alpha-hydroxylase (P450 | 1.2465492 | 0.9045895 | 1.5433869 | 0.573129 | 1.062892 | 1.1226137 |
| VII) | | | | | | |
| Ferritin H-chain | 0.8401744 | 0.889344 | 0.7886416 | 0.8476431 | 0.9178766 | 1.0871491 |
| Phase-1 RCT-197 | 1.0010518 | 1.1075131 | 1.5096287 | 1.3376105 | 0.9881755 | 0.6992306 |
| Phase-1 RCT-179 | 0.9728017 | 1.2646106 | 1.5799181 | 1.7686863 | 1.0316507 | 0.822916 |
| Phase-1 RCT-15 | 1.1571704 | 1.6289351 | 1.7126579 | 2.085583 | 0.7202911 | 1.160184 |
| Argininosuccinate lyase | 1.1721493 | 1.4173967 | 1.207534 | 1.4724733 | 0.7261589 | 1.0349171 |
| Phase-1 RCT-49 | 1.015135 | 0.9109541 | 1.2127489 | 1.3128952 | 1.1193554 | 1.388967 |
| Carbonic anhydrase III | 1.6608068 | 1.1341285 | 0.5711923 | 0.180022 | 0.3137977 | 0.3683957 |
| Proliferating cell nuclear antigen gene | 1.0997511 | 0.996725 | 1.1767169 | 1.1330311 | 1.0434937 | 1.0735391 |
| Integrin beta1 | 1.0296934 | 1.1992862 | 1.3247744 | 0.9196197 | 1.0729389 | 1.0235454 |
| Caspase 6. | 1.028136 | 1.1185081 | 1.1576191 | 1.2105614 | 1.0897089 | 0.9851775 |
| Phase-1 RCT-116 | 1.0913258 | 1.2251989 | 1.0747542 | 1.2412635 | 0.9720064 | 1 |
| Phase-1 RCT-109 | 0.9437618 | 0.9054754 | 1.1708034 | 1.3786588 | 1.2015407 | 1.4353443 |
| Phase-1 RCT-71 | 0.9040546 | 1.1807245 | 1.4632176 | 1.5235918 | 0.9734789 | 0.9047543 |
| Elongation factor-1 alpha | 0.9076935 | 1.0527009 | 1.131851 | 1.1981305 | 0.6923534 | 1.1331064 |
| Phase-1 RCT-169 | 1.1489198 | 0.9414519 | 1.0898005 | 1.2482957 | 1.0145969 | 1.0079296 |
| Pyruvate kinase, muscle | 0.7241072 | 0.9318093 | 1.0223086 | 1.1665587 | 0.5872466 | 1.209805 |
| Nucleosome assembly protein | 1.7215058 | 0.8501829 | 1.1145272 | 0.6596642 | 0.4755126 | 0.3324799 |
| Phase-1 RCT-127 | 1.1018689 | 1.25823 | 1.3076588 | 1.2104185 | 1.0452561 | 1.1627007 |
| Iron-responsive element-binding protein | 0.8791832 | 0.804903 | 0.7009608 | 0.7201752 | 0.8801966 | 0.807871 |
| Phase-1 RCT-72 | 1.0602037 | 1.0097517 | 1.0178484 | 0.9406965 | 0.9806616 | 0.9959475 |
| Phase-1 RCT-144 | 1.0534021 | 1.0354841 | 1.4669204 | 1.6042608 | 1.1374668 | 2.0307443 |
| Phase-1 RCT-242 | 1.1060088 | 0.8610015 | 1.3999615 | 1.6353492 | 1.008149 | 1.1720295 |
| 17-beta hydroxysteroid dehydrogenase, | 1.7808369 | 0.661712 | 0.9546356 | 0.3575856 | 0.2006861 | 0.2039655 |
| type 2 | | | | | | |
| Phase-1 RCT-70 | 0.8815215 | 0.8833354 | 0.8664241 | 0.8530702 | 0.9394416 | 0.9391497 |
| Ribosomal protein L13A | 0.8972626 | 1.129362 | 1.2893931 | 1.2646034 | 1.3203602 | 1.6256821 |
| Cytochrome P450 2E1 | 0.8630563 | 0.5715576 | 0.532455 | 0.5535799 | 1.1365325 | 1.1923635 |
| AT-3 | 0.9758984 | 0.9074341 | 0.9911943 | 0.8142279 | 1.0929141 | 1.0036389 |
| Phase-1 RCT-270 | 0.9479787 | 0.8651555 | 0.6902643 | 0.4929713 | 0.9226681 | 0.6227616 |
| Phase-1 RCT-123 | 1.0511878 | 0.9308649 | 0.9405516 | 0.934152 | 1.0232632 | 1.0069107 |
| Alpha-2-macroglobulin, sequence 2 | 1.2038013 | 1.3822649 | 1.2664762 | 1.4612544 | 1.1068213 | 1.12614 |
| Matrix metalloproteinase-1 | 0.9045153 | 0.9295906 | 0.8764577 | 0.982512 | 1.0788953 | 1.089761 |
| Beta-tubulin, class I | 0.6228005 | 0.5725357 | 0.7599319 | 0.9653844 | 0.9356648 | 1.0712517 |
| Phase-1 RCT-161 | 0.9525347 | 0.8822583 | 0.7153452 | 0.4494319 | 1.0049907 | 0.9844695 |
| 14-3-3 zeta | 1.0312307 | 1.0648961 | 1.0898188 | 1.0773017 | 1.0987899 | 1.0468837 |
| Stem cell factor | 1.3219398 | 0.9651328 | 0.8521899 | 0.6428169 | 1.138411 | 1.1388916 |
| Macrophage inflammatory protein-2 alpha | 1.6472535 | 1.5632253 | 3.163692 | 3.3913941 | 1.1714371 | 1.1485512 |
| Thymidylate synthase | 1.0638106 | 1.062865 | 0.9832365 | 0.9271075 | 1.0311226 | 0.9435151 |
| c-jun | 0.7231992 | 1.0636092 | 1.2081821 | 1.136395 | 0.9659413 | 0.9341424 |
| Superoxide dismutase Mn | 1.1301152 | 0.8298544 | 0.9463896 | 1.6167457 | 1.0590942 | 1.2505109 |
| Phase-1 RCT-73 | 1.0596572 | 0.9114673 | 1.0039325 | 0.9140292 | 0.912039 | 0.8499407 |
| Macrophage inflammatory protein-1 alpha | 1.0012385 | 1.0678896 | 0.9425554 | 1.1537448 | 1.0747093 | 1.0173438 |
| Phase-1 RCT-214 | 0.9196772 | 0.6234576 | 0.6201433 | 0.54618 | 1.0316507 | 0.9055615 |
| NADH-cytochrome b5 reductase | 0.7455637 | 0.7221691 | 0.5807792 | 0.4906566 | 0.7668027 | 0.6613845 |
| Interleukin-1 beta | 0.9073124 | 0.9022868 | 0.8463323 | 0.9268324 | 1.1905206 | 1.1433368 |
| NADP-dependent isocitrate | 0.7810223 | 0.6321428 | 0.5643269 | 0.5092659 | 0.9866183 | 0.9812557 |
| dehydrogenase, cytosolic | | | | | | |
| Phase-1 RCT-289 | 0.9634334 | 0.8108795 | 0.7658076 | 0.7665552 | 0.6439639 | 0.813585 |
| Glutamine synthetase | 1.0803361 | 1.3987229 | 1.7521758 | 1.1696112 | 1.0387564 | 1.243397 |
| Phase-1 RCT-182 | 1.1764693 | 0.9417486 | 0.7676665 | 0.6385971 | 0.7269443 | 0.7956127 |
| Ubiquitin conjugating enzyme (RAD 6 | 1.1468316 | 1.11947 | 1.3307228 | 1.3360448 | 1.4110401 | 0.9231269 |
| homologue) | | | | | | |
| Extracellular-signal-regulated kinase 1 | 1.6081673 | 1.1245155 | 0.7396845 | 0.4659215 | 1.097133 | 0.7860098 |
| Emerin | 0.8942308 | 0.843747 | 0.8888914 | 0.8373954 | 0.9292651 | 0.8438871 |
| Phase-1 RCT-78 | 1.0758878 | 0.9047376 | 0.7439705 | 0.7787119 | 0.9847403 | 0.9336149 |
| Phase-1 RCT-212 | 0.7839279 | 0.9503546 | 0.9676265 | 0.9860859 | 1.0212438 | 1.0061414 |
| Phase-1 RCT-168 | 0.7628744 | 0.7219511 | 0.7088559 | 0.5709583 | 0.8891723 | 0.5998948 |
| Lysyl hydroxylase | 1.1273719 | 1.1075172 | 1.3210288 | 1.125713 | 0.9728623 | 1.1593138 |
| Phase-1 RCT-55 | 0.651376 | 0.6732449 | 0.9040576 | 0.8420354 | 1.0488449 | 1.0537916 |
| MHC class I antigen RT1.A1(f) alpha- | 0.9529482 | 0.9644675 | 1.0865027 | 1.0817662 | 0.4611456 | 0.420739 |
| chain | | | | | | |
| Phase-1 RCT-40 | 0.8990918 | 0.8520672 | 0.8283281 | 0.7719045 | 0.9577521 | 0.8987123 |
| Cyclin G | 0.9857627 | 0.7482751 | 0.9870411 | 1.3463203 | 1.1237624 | 1.0946981 |
| Peroxisome proliferator activated receptor | 1.0792725 | 0.9990219 | 0.9674143 | 0.8224748 | 1.0909039 | 1.0335038 |
| gamma | | | | | | |
| Phase-1 RCT-280 | 1.1296688 | 0.9681054 | 0.9307225 | 0.8516575 | 0.8594139 | 1.0939612 |
| D-dopachrome tautomerase | 0.8349549 | 0.7522583 | 0.6958076 | 0.666018 | 0.8620644 | 0.8396218 |
| Phase-1 RCT-287 | 0.9742486 | 0.788684 | 0.8273925 | 0.6667829 | 1.0045755 | 0.9854103 |
| Phase-1 RCT-119 | 1.2203325 | 1.5088385 | 1.0357172 | 1.1789049 | 1.0091828 | 0.9959831 |
| Thioredoxin-1 (Trx1) | 1.0298781 | 0.9835901 | 1.1378111 | 1.0943652 | 0.8792204 | 0.8463008 |
| Phase-1 RCT-225 | 0.6906859 | 1.0495203 | 0.9392533 | 0.8982911 | 0.8693634 | 0.603808 |
| Protein kinase C alpha | 0.9885746 | 1.0677347 | 1.0984975 | 1.0951512 | 0.940594 | 1.1256125 |
| Phase-1 RCT-87 | 0.851605 | 0.8277987 | 0.9154398 | 0.781625 | 1.0003433 | 1.1352594 |
| Phase-1 RCT-59 | 1.0750806 | 0.9478618 | 0.9244025 | 1.0301212 | 0.9119055 | 0.8848243 |
| C4b-binding protein | 1.152354 | 1.0763485 | 1.0389904 | 1.0676023 | 0.9999718 | 0.8604266 |
| Phase-1 RCT-204 | 1.0275985 | 1.0440515 | 0.9439316 | 0.9641559 | 1.0407457 | 1.1550591 |
| Carnitine palmitoyl-CoA transferase | 1.0338005 | 0.8877612 | 0.7674275 | 0.9178608 | 0.9617786 | 0.6582729 |
| 8-oxoguanine DNA glycosylase | 0.9279355 | 1.0716586 | 1.0491873 | 0.9273238 | 1.0881871 | 1.1418774 |
| H-rev107 | 0.9800145 | 0.83538 | 0.7509936 | 0.9673784 | 1.0028638 | 0.8998985 |
| Preproalbumin | 1.1997442 | 1.0094185 | 0.7840186 | 0.7145684 | 0.2967645 | 0.6155893 |
| Phase-1 RCT-177 | 0.98301 | 0.9464024 | 1.0867333 | 0.9740524 | 0.7817478 | 0.780426 |
| Phase-1 RCT-110 | 1.0585583 | 0.9061975 | 0.992923 | 0.9416543 | 1.0107212 | 1.0111414 |
| Phase-1 RCT-227 | 1.0613592 | 1.1006259 | 0.6467739 | 0.6465168 | 0.7562874 | 0.8762425 |
| Neuropeptide Y | 1.1084256 | 1.0189542 | 0.9165136 | 1.0301815 | 1.0439323 | 1.0468837 |
| Phase-1 RCT-277 | 0.3990001 | 0.6206554 | 0.5329996 | 1.0717953 | 0.9950581 | 1.0435135 |
| Phase-1 RCT-229 | 1.0259573 | 1.0219699 | 0.9527692 | 0.9439368 | 1.029568 | 1.0506895 |
| Phase-1 RCT-34 | 0.8661957 | 0.7248761 | 0.6439076 | 0.4843302 | 0.9899812 | 0.9068196 |
| Choline kinase | 1.2703842 | 1.0927864 | 1.037551 | 1.0268825 | 1.1522398 | 1.1482743 |
| Phase-1 RCT-20 | 1.035019 | 1.1245399 | 1.1100212 | 1.0298755 | 0.9614413 | 0.9180853 |
| Phase-1 RCT-248 | 1.0191532 | 0.8057694 | 0.8051589 | 1.0688107 | 1.0702499 | 1.1514677 |
| | TET | TET | TET | TET | THEO | THEO |
| Compound-Dose (2) | 50 | 150 | 150 | 150 | 25 | 25 |
| Animal Number (3) | 123 | 1241 | 1242 | 1243 | 2521 | 2522 |
| Liver Toxicity Necrosis Classification (4) | no | yes | yes | yes | no | no |
| Gene Name (5) | | | | | | |
| Gadd153 | 1.7287107 | 1.6459739 | 2.586845 | 2.2226994 | 0.7752263 | 1.1924525 |
| Interferon related developmental regulator | 1.0555514 | 2.97996 | 2.3007605 | 2.1452572 | 0.987623 | 0.9877742 |
| IFRD1 (PC4) | | | | | | |
| ID-1 | 1.1019382 | 1.0753717 | 1.3898617 | 1.1907985 | 0.6845145 | 0.7978036 |
| Insulin-like growth factor binding protein 1 | 2.2526062 | 23.46054 | 3.8853085 | 4.5951953 | 0.7763941 | 0.9261805 |
| NIPK | 1.4669368 | 6.4005113 | 1.386775 | 1.4023478 | 0.8020333 | 0.882137 |
| Phase-1 RCT-50 | 1.3421224 | 3.2272909 | 1.4457723 | 1.3720336 | 0.7872099 | 0.9865749 |
| Sarcoplasmic reticulum calcium ATPase | 1.1613445 | 0.8061075 | 0.782101 | 0.6127147 | 0.8668022 | 1.1027774 |
| Phase-1 RCT-207 | 0.9361104 | 2.9878724 | 1.0893409 | 0.9890544 | 0.9562609 | 1.0277696 |
| Acetyl-CoA carboxylase | 1.0946552 | 1.4675243 | 1.0144334 | 0.994868 | 0.9713193 | 0.9311653 |
| Cathepsin L, sequence 2 | 0.1088762 | 2.4765198 | 2.8016455 | 2.2516057 | 0.9004124 | 1.339304 |
| Glucokinase | 1.0091162 | 0.1726747 | 0.6399636 | 0.5122083 | 0.4382007 | 1.0890862 |
| Gadd45 | 1.0645863 | 1.648844 | 2.7049263 | 4.038101 | 1.1666389 | 1.0466483 |
| Phase-1 RCT-18 | 1.0912548 | 0.7465744 | 0.8830207 | 0.8215699 | 0.8662741 | 0.9190651 |
| 3-beta-hydroxysteroid dehydrogenase | 0.8278001 | 1.2895063 | 1.3983064 | 1.1869123 | 1.1011038 | 0.9576808 |
| (HSD3B1) | | | | | | |
| Phase-1 RCT-221 | 0.9876384 | 1.3006711 | 0.8061788 | 0.8524496 | 0.9393513 | 0.9652193 |
| Heme oxygenase | 1.6177213 | 1.598419 | 2.06196 | 3.041665 | 1.0568699 | 1.0359479 |
| Cyclin dependent kinase 4 | 1.4400903 | 0.9394653 | 0.9426039 | 0.9230396 | 1.0625459 | 1.1226432 |
| Phase-1 RCT-75 | 1.3428543 | 2.0255203 | 1.5425689 | 1.4505416 | 1.3067768 | 1.0070978 |
| Phase-1 RCT-65 | 0.6202197 | 0.9906632 | 0.8179236 | 1.013863 | 1.1200975 | 0.9437748 |
| Cytochrome P450 1A1 | 1.095325 | 0.5504068 | 0.4286065 | 0.3211366 | 0.6940336 | 1.2566228 |
| Peroxisomal multifunctional enzyme type II | 0.9635738 | 1.2593834 | 1.1665587 | 1.1636746 | 1.3865554 | 1.0118332 |
| DNA topoisomerase I | 1.2329654 | 1.3153661 | .1.36883 | 1.303115 | 1.2868818 | 0.9504618 |
| Focal adhesion kinase (pp125FAK) | 1.0712708 | 1.2893726 | 1.3287659 | 1.1674271 | 1.0731168 | 1.142466 |
| NGF-inducible anti-proliferative putative | 1.1317866 | 5.7680564 | 1.7429188 | 1.5740573 | 1.2051606 | 0.959933 |
| secreted protein (PC3) | | | | | | |
| Phase-1 RCT-82 | 1.0147123 | 0.3662282 | 0.8019152 | 0.7815983 | 0.8800565 | 0.9463711 |
| Senescence marker protein-30 | 0.8404867 | 0.2795345 | 0.3785799 | 0.5033277 | 1.1814433 | 0.8658488 |
| Melanoma-associated antigen ME491 | 1.4199691 | 2.0501099 | 1.7088709 | 1.1281859 | 1.1837671 | 0.9897392 |
| Activating transcription factor 3 | 0.7914349 | 2.0199034 | 0.8646992 | 0.965238 | 1.0987115 | 1.0375614 |
| Tryptophan hydroxylase | 0.8006082 | 0.6327201 | 0.7946869 | 0.6273946 | 1.1699303 | 0.8662917 |
| Zinc finger protein | 0.9994335 | 5.868788 | 1.2830467 | 1.307543 | 0.8342457 | 0.9410725 |
| c-myc | 1.2298065 | 4.240596 | 2.251538 | 2.1081502 | 0.8519322 | 1.0658635 |
| Cholesterol 7-alpha-hydroxylase (P450 | 1.0375427 | 0.4362364 | 0.5093055 | 0.7561398 | 0.7853536 | 1.6901469 |
| VII) | | | | | | |
| Ferritin H-chain | 1.1171178 | 0.9458926 | 1.4246433 | 1.4388057 | 0.9142672 | 0.9344307 |
| Phase-1 RCT-197 | 0.9203821 | 1.0991192 | 1.5332628 | 1.2428591 | 0.8666908 | 1.0374818 |
| Phase-1 RCT-179 | 0.9616041 | 2.9299695 | 2.8949263 | 2.3344326 | 0.9479708 | 1.1859794 |
| Phase-1 RCT-15 | 1.0666533 | 3.7141626 | 1.5848471 | 1.8093436 | 1.1192573 | 1.0861093 |
| Argininosuccinate lyase | 0.8636115 | 2.429773 | 1.6964365 | 2.3832288 | 1.8577434 | 1.0167589 |
| Phase-1 RCT-49 | 1.3196394 | 3.5828943 | 1.6736058 | 1.9922246 | 1.0798991 | 1.0161589 |
| Carbonic anhydrase III | 0.495383 | 0.355884 | 0.2124466 | 0.2839461 | 1.2589093 | 0.4726734 |
| Proliferating cell nuclear antigen gene | 1.1469014 | 1.4021624 | 1.427327 | 1.4506018 | 1.0867715 | 1.0703213 |
| Integrin beta1 | 1.0216045 | 3.1595175 | 2.2417653 | 1.8436278 | 0.9914379 | 0.9792684 |
| Caspase 6 | 0.986119 | 0.8888151 | 1.2106845 | 1.0138583 | 0.9418715 | 0.9562995 |
| Phase-1 RCT-116 | 1.0885096 | 1.4422684 | 1.4529233 | 1.8083924 | 0.8650974 | 0.9423398 |
| Phase-1 RCT-109 | 1.4603536 | 1.5643387 | 1.167983 | 1.2676982 | 0.9743932 | 0.9339277 |
| Phase-1 RCT-71 | 0.943261 | 1.4141116 | 1.0318687 | 1.0447232 | 1.1502593 | 1.1855267 |
| Elongation factor-1 alpha | 1.1147811 | 1.40229 | 1.5782937 | 1.3578689 | 0.9739652 | 1.0361316 |
| Phase-1 RCT-169 | 0.987298 | 2.5794394 | 1.591515 | 1.3402148 | 0.9055523 | 0.9662483 |
| Pyruvate kinase, muscle | 1.1642039 | 0.9806974 | 2.2459695 | 1.7085382 | 0.8595308 | 1.1542966 |
| Nucleosome assembly protein | 0.362669 | 0.618241 | 0.7025522 | 1.258526 | 0.8543731 | 0.9623375 |
| Phase-1 RCT-127 | 1.0819112 | 3.1272588 | 0.73121 | 0.9245081 | 0.8657306 | 0.9806012 |
| Iron-responsive element-binding protein | 0.7562185 | 0.7433924 | 0.6960751 | 0.7689934 | 1.1788161 | 1.2229723 |
| Phase-1 RCT-72 | 1.0880735 | 0.5626981 | 0.8767434 | 0.8413168 | 0.8381942 | 1.0144036 |
| Phase-1 RCT-144 | 1.6395702 | 3.626966 | 2.0350773 | 1.8029693 | 1.2085899 | 1.1826333 |
| Phase-1 RCT-242 | 1.076548 | 9.684165 | 1.4815421 | 1.4925084 | 0.9431441 | 0.9746188 |
| 17-beta hydroxysteroid dehydrogenase, | 0.2010636 | 0.2802385 | 0.4984455 | 1.6870192 | 0.8131833 | 0.9443286 |
| type 2 | | | | | | |
| Phase-1 RCT-70 | 0.7058498 | 0.6533885 | 0.8507652 | 0.74724 | 1.0160468 | 0.7933176 |
| Ribosomal protein L13A | 1.6767935 | 1.4493297 | 1.3918498 | 1.4101118 | 0.9967002 | 1.0643643 |
| Cytochrome P450 2E1 | 1.1242486 | 0.4376164 | 0.6069357 | 0.6722736 | 0.5736742 | 1.2321931 |
| AT-3 | 1.0939116 | 1.1195611 | 1.174079 | 1.1561028 | 0.8158385 | 0.9182785 |
| Phase-1 RCT-270 | 0.6781825 | 0.331263 | 0.4515843 | 0.4193378 | 1.21691 | 0.7282107 |
| Phase-1 RCT-123 | 1.099863 | 1.0566293 | 1.0515664 | 1.1778936 | 0.8836514 | 1.0060465 |
| Alpha-2-macroglobulin, sequence 2 | 1.0791031 | 2.0648744 | 1.9354894 | 1.6620501 | 1.1180482 | 1.0767897 |
| Matrix metalloproteinase-1 | 1.1871523 | 0.792479 | 1.3650472 | 1.3103944 | 1.0567423 | 0.9469835 |
| Beta-tubulin, class I | 1.1359575 | 0.8154547 | 1.2591362 | 1.2112389 | 1.2152667 | 1.073731 |
| Phase-1 RCT-161 | 0.985404 | 0.3547162 | 0.9227314 | 0.8253828 | 0.7848021 | 0.5986416 |
| 14-3-3 zeta | 1.0924345 | 1.7278771 | 1.6708562 | 1.5667573 | 1.1121051 | 1.1293842 |
| Stem cell factor | 1.1309311 | 0.3122962 | 0.5986137 | 0.5058357 | 0.8328635 | 0.8685027 |
| Macrophage inflammatory protein-2 alpha | 1.1319925 | 2.8700662 | 1.01659 | 0.8137181 | 0.7895597 | 1.058721 |
| Thymidylate synthase | 1.129138 | 0.8720717 | 0.8165292 | 0.7861435 | 0.899439 | 1.1111943 |
| c-jun | 0.4683426 | 0.6643291 | 0.7262391 | 0.8852158 | 0.8047538 | 1.4232118 |
| Superoxide dismutase Mn | 1.1911262 | 4.0748386 | 6.1163354 | 3.9385533 | 1.1978381 | 0.9895366 |
| Phase-1 RCT-73 | 0.8894822 | 0.9585655 | 1.2285091 | 1.3546143 | 1.1383699 | 1.0425117 |
| Macrophage inflammatory protein-1 alpha | 0.985906 | 2.6214674 | 1.5245483 | 1.247878 | 0.7525905 | 0.9752287 |
| Phase-1 RCT-214 | 1.0152906 | 0.3357716 | 0.7282479 | 0.8508155 | 0.6860188 | 0.9382326 |
| NADH-cytochrome b5 reductase | 0.7351604 | 0.5274558 | 0.5250855 | 0.5764589 | 1.2689832 | 1.031508 |
| Interleukin-1 beta | 1.2719836 | 0.7638702 | 1.3719634 | 1.2384241 | 0.9366627 | 0.9925091 |
| NADP-dependent isocitrate | 0.9609651 | 0.3768987 | 0.5725113 | 0.6310725 | 1.3144648 | 0.9783028 |
| dehydrogenase, cytosolic | | | | | | |
| Phase-1 RCT-289 | 0.8244113 | 0.8772255 | 0.8329076 | 0.8979993 | 1.068664 | 1.1137966 |
| Glutamine synthetase | 1.2391644 | 2.21418 | 1.4192013 | 1.2165644 | 1.590084 | 0.8564124 |
| Phase-1 RCT-182 | 0.6884282 | 0.7698764 | 0.5891542 | 0.6466979 | 1.0755646 | 0.7887591 |
| Ubiquitin conjugating enzyme (RAD 6 | 1.1342868 | 2.4847426 | 2.1554332 | 2.0340924 | 1.1096836 | 1.1654197 |
| homologue) | | | | | | |
| Extracellular-signal-regulated kinase 1 | 0.652741 | 0.5826122 | 0.2098335 | 0.2867657 | 1.1014205 | 0.5311538 |
| Emerin | 0.882223 | 1.0141357 | 0.7976743 | 0.8812617 | 1.0019686 | 1.066056 |
| Phase-1 RCT-78 | 0.8479589 | 0.8712426 | 0.7962941 | 0.6794639 | 0.9730215 | 0.8549288 |
| Phase-1 RCT-212 | 1.1609261 | 0.8633462 | 0.9230196 | 0.7608674 | 1.0761214 | 1.1818571 |
| Phase-1 RCT-168 | 0.7698506 | 0.354471 | 0.6975219 | 0.6754807 | 1.0584272 | 1.031299 |
| Lysyl hydroxylase | 1.1136639 | 1.5474619 | 1.2623758 | 1.0489478 | 1.1832788 | 1.0727407 |
| Phase-1 RCT-55 | 1.0531853 | 0.8916424 | 0.6834844 | 0.6411634 | 1.1299388 | 1.1210498 |
| MHC class I antigen RT1.A1(f) alpha- | 0.3560063 | 0.6185644 | 0.9295398 | 1.2161596 | 1.1808603 | 0.9119438 |
| chain | | | | | | |
| Phase-1 RCT-40 | 0.8720716 | 0.7079805 | 0.9545589 | 0.865555 | 1.0881213 | 0.878525 |
| Cyclin G | 1.233938 | 2.4413002 | 2.4921587 | 1.3049314 | 1.493512 | 1.1726016 |
| Peroxisome proliferator activated receptor | 1.1193396 | 1.3539121 | 1.2006408 | 1.067273 | 0.8045256 | 1.2332311 |
| gamma | | | | | | |
| Phase-1 RCT-280 | 1.1654886 | 1.1687623 | 1.5020163 | 1.7023355 | 0.7940206 | 0.9688684 |
| D-dopachrome tautomerase | 0.908399 | 0.7437282 | 0.8814949 | 0.9539688 | 1.267419 | 1.038328 |
| Phase-1 RCT-287 | 1.0659937 | 0.968357 | 1.0886254 | 0.9886358 | 1.0711565 | 0.9046877 |
| Phase-1 RCT-119 | 0.9880701 | 0.6405414 | 0.7311688 | 1.074997 | 1.0987856 | 0.8350776 |
| Thioredoxin-1 (Trx1) | 0.8772386 | 1.0355676 | 1.5046414 | 1.4396245 | 1.1425707 | 0.981987 |
| Phase-1 RCT-225 | 0.6659025 | 0.3949658 | 0.5094572 | 0.5719407 | 0.8893925 | 1.3970246 |
| Protein kinase C alpha | 0.9869801 | 0.9417291 | 0.8132979 | 0.8065043 | 0.9065256 | 1.10531 |
| Phase-1 RCT-87 | 1.1252395 | 0.5992389 | 0.9688637 | 0.9549139 | 1.155893 | 1.0210634 |
| Phase-1 RCT-59 | 1.0421901 | 2.9625936 | 1.0366244 | 0.9679333 | 0.7687917 | 1.0484384 |
| C4b-binding protein | 1.5424225 | 0.6305668 | 1.846223 | 1.4642987 | 1.3067627 | 0.9717332 |
| Phase-1 RCT-204 | 1.183612 | 0.9976274 | 1.3176935 | 1.4610274 | 1.0580602 | 1.0160569 |
| Carnitine palmitoyl-CoA transferase | 0.8344178 | 1.6889536 | 0.8078581 | 1.6294596 | 0.8705062 | 0.8666874 |
| 8-oxoguanine DNA glycosylase | 1.1355017 | 1.1671425 | 0.49547 | 0.4766083 | 0.8559238 | 1.1389185 |
| H-rev107 | 1.0824822 | 0.6467125 | 0.9057511 | 0.3730438 | 1.1665697 | 1.057679 |
| Preproalbumin | 0.5842912 | 0.8676387 | 1.5335246 | 1.5956192 | 1.3258455 | 0.9900456 |
| Phase-1 RCT-177 | 0.7062671 | 0.9969268 | 1.3492973 | 1.6250275 | 1.3673252 | 1.0770427 |
| Phase-1 RCT-110 | 1.0784268 | 1.0484556 | 0.8983942 | 0.8088272 | 0.8629248 | 0.9440637 |
| Phase-1 RCT-227 | 0.5957248 | 1.0472249 | 0.8709276 | 0.7783664 | 1.2691003 | 0.8782275 |
| Neuropeptide Y | 1.1807181 | 0.9489014 | 1.0389317 | 0.9147463 | 0.9411258 | 1.0485665 |
| Phase-1 RCT-277 | 0.9900913 | 0.8231311 | 0.9287719 | 0.8723901 | 1.0956188 | 1.8018144 |
| Phase-1 RCT-229 | 1.0819112 | 0.7573872 | 0.8176934 | 0.7733643 | 0.8000171 | 0.8712264 |
| Phase-1 RCT-34 | 0.8574428 | 0.2728113 | 0.698128 | 0.667824 | 1.1486567 | 1.0831641 |
| Choline kinase | 1.1593593 | 0.4911241 | 0.8793119 | 1.0698757 | 0.7811079 | 0.770677 |
| Phase-1 RCT-20 | 0.8274643 | 0.3696094 | 1.1110257 | 0.9545777 | 0.8381421 | 1.0417258 |
| Phase-1 RCT-248 | 1.1921928 | 1.1631304 | 1.075939 | 1.1317575 | 0.8252632 | 0.9709334 |

| | THEO | THEO | THEO | THEO |
|---|---|---|---|---|
| Compound-Dose (2) | 25 | 100 | 100 | 100 |
| Animal Number (3) | 2523 | 2531 | 2532 | 2533 |
| Liver Toxicity Necrosis Classification (4) | no | no | no | no |
| Gene Name (5) | | | | |
| Gadd153 | 1.0757678 | 0.9531249 | 1.2840645 | 0.8543842 |
| Interferon related developmental regulator | 1.1450814 | 1.287171 | 1.5135175 | 1.7927483 |
| IFRD1 (PC4) | | | | |
| ID-1 | 0.820941 | 1.662615 | 1.5134492 | 1.0961545 |
| Insulin-like growth factor binding protein 1 | 1.1271468 | 11.388036 | 1.8577055 | 1.0195587 |
| NIPK | 0.9586394 | 1.2178138 | 1.0404977 | 1.0693463 |
| Phase-1 RCT-50 | 0.8988475 | 3.4928575 | 1.7231 | 0.9665522 |
| Sarcoplasmic reticulum calcium ATPase | 1.0802929 | 1.1523994 | 1.4429387 | 1.2799648 |
| Phase-1 RCT-207 | 1.0204151 | 2.0221515 | 2.1250558 | 2.0207424 |
| Acetyl-CoA carboxylase | 0.9759992 | 1.2608329 | 1.5450202 | 2.034776 |
| Cathepsin L, sequence 2 | 1.1568261 | 1.6367413 | 1.6378412 | 1.9477276 |
| Glucokinase | 0.7779893 | 0.227183 | 0.2231991 | 0.325073 |
| Gadd45 | 1.150977 | 2.5452342 | 2.2720706 | 1.2665205 |
| Phase-1 RCT-18 | 0.8999034 | 1.0880953 | 1.0562044 | 1.0352442 |
| 3-beta-hydroxysteroid dehydrogenase | 0.669427 | 0.6202604 | 0.6841856 | 0.790359 |
| (HSD3B1) | | | | |
| Phase-1 RCT-221 | 1.0064441 | 1.0301074 | 1.0854243 | 0.874207 |
| Heme oxygenase | 1.4599674 | 3.2060776 | 5.717468 | 2.1466641 |
| Cyclin dependent kinase 4 | 1.1450939 | 0.8006824 | 0.9631147 | 1.0132068 |
| Phase-1 RCT-75 | 1.2262719 | 1.2522854 | 1.2295071 | 1.4222938 |
| Phase-1 RCT-65 | 0.9676945 | 0.4320325 | 0.4145742 | 0.3801141 |
| Cytochrome P450 1A1 | 0.8530051 | 1.4558653 | 1.155507 | 1.1110605 |
| Peroxisomal multifunctional enzyme type II | 1.2059176 | 0.658994 | 0.8394799 | 0.8796246 |
| DNA topoisomerase I | 1.3364993 | 1.1016858 | 1.3263638 | 1.1424066 |
| Focal adhesion kinase (pp125FAK) | 1.2192931 | 1.3414066 | 1.2758701 | 1.1721156 |
| NGF-inducible anti-proliferative putative | 1.1530159 | 4.0344834 | 1.3795621 | 0.8526026 |
| secreted protein (PC3) | | | | |
| Phase-1 RCT-82 | 0.8807252 | 0.8659267 | 0.8330126 | 0.8465989 |
| Senescence marker protein-30 | 1.256244 | 0.2502691 | 0.3005393 | 0.3606139 |
| Melanoma-associated antigen ME491 | 0.9775848 | 1.2712712 | 1.3453335 | 1.1431388 |
| Activating transcription factor 3 | 0.9684395 | 1.0077639 | 0.8186535 | 0.8512373 |
| Tryptophan hydroxylase | 1.1195056 | 0.597977 | 0.696626 | 0.8788759 |
| Zinc finger protein | 0.8164806 | 2.7797558 | 2.394884 | 1.3506024 |
| c-myc | 1.0058763 | 3.7597053 | 2.3545673 | 1.0720751 |
| Cholesterol 7-alpha-hydroxylase (P450 | 1.1785723 | 1.1003965 | 0.914404 | 0.9928329 |
| VII) | | | | |
| Ferritin H-chain | 1.0503954 | 0.7044287 | 0.7879279 | 0.6523632 |
| Phase-1 RCT-197 | 1.0427165 | 0.7564947 | 0.8586181 | 1.1882805 |
| Phase-1 RCT-179 | 1.3177061 | 2.4692686 | 2.7024078 | 1.8806285 |
| Phase-1 RCT-15 | 1.1902155 | 2.2042453 | 1.7152029 | 1.8299277 |
| Argininosuccinate lyase | 1.0717933 | 1.9938915 | 1.7210898 | 2.0828123 |
| Phase-1 RCT-49 | 1.1728091 | 2.1705317 | 2.4710927 | 2.0702784 |
| Carbonic anhydrase III | 0.336306 | 0.0952575 | 0.2038679 | 0.1489183 |
| Proliferating cell nuclear antigen gene | 1.0665827 | 1.4294137 | 1.4133391 | 1.4064122 |
| Integrin beta1 | 1.0015672 | 7.585116 | 1.7902143 | 1.2068578 |
| Caspase 6 | 0.9694789 | 1.0693247 | 0.7514862 | 0.7619633 |
| Phase-1 RCT-116 | 0.7949851 | 1.0562338 | 1.029916 | 1.0601768 |
| Phase-1 RCT-109 | 1.1159103 | 1.1545151 | 1.1472744 | 0.978056 |
| Phase-1 RCT-71 | 1.1442984 | 1.6052722 | 1.339217 | 1.6004844 |
| Elongation factor-1 alpha | 1.041973 | 0.8964942 | 1.1922263 | 0.9392347 |
| Phase-1 RCT-169 | 0.7526875 | 0.4548971 | 0.5900487 | 0.5647399 |
| Pyruvate kinase, muscle | 1.0015913 | 1.425564 | 1.419108 | 1.4972262 |
| Nucleosome assembly protein | 0.9558404 | 0.5505292 | 0.5366014 | 0.6443425 |
| Phase-1 RCT-127 | 1.1231229 | 2.0728037 | 2.3874643 | 1.8279389 |
| Iron-responsive element-binding protein | 1.2326317 | 0.6971393 | 0.5931574 | 0.8605114 |
| Phase-1 RCT-72 | 0.861345 | 1.1306155 | 1.1636096 | 1.092996 |
| Phase-1 RCT-144 | 1.442581 | 2.119773 | 2.3132586 | 1.723946 |
| Phase-1 RCT-242 | 0.9148597 | 9.1628475 | 2.615471 | 1.2044868 |
| 17-beta hydroxysteroid dehydrogenase, | 0.9913685 | 0.2137981 | 0.2331921 | 0.2327655 |
| type 2 | | | | |
| Phase-1 RCT-70 | 0.9031069 | 0.9836611 | 0.885477 | 1.0187027 |
| Ribosomal protein L13A | 1.1144984 | 1.1840187 | 1.2628536 | 1.0602447 |
| Cytochrome P450 2E1 | 0.7706027 | 0.6134111 | 0.7337926 | 0.7952657 |
| AT-3 | 0.8884725 | 0.9496468 | 0.9116857 | 0.9323819 |
| Phase-1 RCT-270 | 0.7656892 | 0.2598354 | 0.2977355 | 0.4287843 |
| Phase-1 RCT-123 | 1.1400163 | 0.942926 | 0.9165151 | 0.8891025 |
| Alpha-2-macroglobulin, sequence 2 | 0.9436341 | 4.1839075 | 2.5233362 | 2.715508 |
| Matrix metalloproteinase-1 | 1.1383064 | 0.8762247 | 0.9226188 | 0.779699 |
| Beta-tubulin, class I | 1.4651042 | 0.8522539 | 0.8473058 | 0.6011008 |
| Phase-1 RCT-161 | 0.79948 | 1.0039079 | 0.9406388 | 0.9734567 |
| 14-3-3 zeta | 0.9514876 | 1.4999998 | 1.131468 | 0.9499575 |
| Stem cell factor | 1.0279288 | 0.3938148 | 0.5186146 | 0.5154229 |
| Macrophage inflammatory protein-2 alpha | 0.8434629 | 0.9516312 | 1.0026371 | 0.9322481 |
| Thymidylate synthase | 1.0587991 | 1.0648315 | 1.011496 | 1.0960153 |
| c-jun | 0.7525032 | 2.6571016 | 1.4275053 | 1.0407388 |
| Superoxide dismutase Mn | 1.1507727 | 9.522318 | 8.430987 | 2.6703746 |
| Phase-1 RCT-73 | 1.1391124 | 0.581481 | 0.7147318 | 0.8293513 |
| Macrophage inflammatory protein-1 alpha | 0.7763016 | 1.5353768 | 1.1271933 | 1.0955105 |
| Phase-1 RCT-214 | 0.9780941 | 0.3424636 | 0.4287686 | 0.4825276 |
| NADH-cytochrome b5 reductase | 0.8872259 | 0.458207 | 0.433095 | 0.6039734 |
| Interleukin-1 beta | 0.9582817 | 1.1757431 | 1.057491 | 0.996048 |
| NADP-dependent isocitrate | 1.1305422 | 0.2265338 | 0.2320493 | 0.4220586 |
| dehydrogenase, cytosolic | | | | |
| Phase-1 RCT-289 | 1.0042347 | 0.528327 | 0.5905693 | 0.7509664 |
| Glutamine synthetase | 0.8273197 | 0.6917988 | 0.7057206 | 1.2005938 |
| Phase-1 RCT-182 | 0.7068199 | 0.3807657 | 0.4469583 | 0.5662849 |
| Ubiquitin conjugating enzyme (RAD 6 | 0.9852318 | 1.4229134 | 1.7556481 | 1.4431288 |
| homologue) | | | | |
| Extracellular-signal-regulated kinase 1 | 0.3979802 | 0.8091687 | 0.3809018 | 0.2992182 |
| Emerin | 1.0296068 | 0.9470561 | 0.9017744 | 0.9099938 |
| Phase-1 RCT-78 | 0.7926453 | 0.6633263 | 0.7515341 | 0.757053 |
| Phase-1 RCT-212 | 0.9673836 | 1.0145875 | 1.0426927 | 1.1432428 |
| Phase-1 RCT-168 | 1.0356401 | 0.4757742 | 0.4110178 | 0.4683473 |
| Lysyl hydroxylase | 0.9938495 | 0.8714587 | 0.9385527 | 1.0255687 |
| Phase-1 RCT-55 | 0.8445901 | 1.3403332 | 1.5320877 | 1.5006536 |
| MHC class I antigen RT1.A1(f) alpha- | 1.1856972 | 1.504269 | 0.88223 | 0.8514664 |
| chain | | | | |
| Phase-1 RCT-40 | 0.9822658 | 0.4853818 | 0.5595729 | 0.5963956 |
| Cyclin G | 1.2441028 | 2.2264364 | 1.9064409 | 1.5431788 |
| Peroxisome proliferator activated receptor | 1.1127053 | 0.9407037 | 0.9634767 | 0.9402155 |
| gamma | | | | |
| Phase-1 RCT-280 | 0.939752 | 0.8991229 | 1.5064856 | 0.7863306 |
| D-dopachrome tautomerase | 1.0062424 | 0.3894646 | 0.3965566 | 0.5014159 |
| Phase-1 RCT-287 | 1.046466 | 0.721317 | 0.7420956 | 0.8771454 |
| Phase-1 RCT-119 | 0.8332445 | 1.1050906 | 1.2604584 | 1.7668016 |
| Thioredoxin-1 (Trx1) | 1.0823907 | 1.0434809 | 1.2330936 | 1.046098 |
| Phase-1 RCT-225 | 0.949782 | 2.0354884 | 1.5642136 | 0.7874278 |
| Protein kinase C alpha | 0.9094369 | 1.1954852 | 1.1573874 | 1.0430955 |
| Phase-1 RCT-87 | 1.0168767 | 0.7201638 | 0.7304875 | 0.9904006 |
| Phase-1 RCT-59 | 1.0540829 | 2.2288082 | 2.3831477 | 2.4686112 |
| C4b-binding protein | 1.5207027 | 0.8285646 | 1.2686068 | 1.0474222 |
| Phase-1 RCT-204 | 1.0772231 | 0.8582717 | 1.0403843 | 0.9962844 |
| Carnitine palmitoyl-CoA transferase | 0.94997 | 0.9747372 | 1.0556021 | 1.088 |
| 8-oxoguanine DNA glycosylase | 0.9919823 | 1.0716525 | 1.0506706 | 1.1156777 |
| H-rev107 | 1.259636 | 1.0613165 | 0.8983872 | 0.9394816 |
| Preproalbumin | 1.0389938 | 0.7020814 | 0.5825915 | 0.8190556 |
| Phase-1 RCT-177 | 1.3796384 | 0.405783 | 0.6331104 | 0.763036 |
| Phase-1 RCT-110 | 0.9662713 | 1.0334934 | 0.9720579 | 0.9945264 |
| Phase-1 RCT-227 | 0.6984144 | 0.607359 | 0.6451422 | 0.7021602 |
| Neuropeptide Y | 1.3406657 | 1.0645744 | 1.1881795 | 1.1931964 |
| Phase-1 RCT-277 | 1.6261044 | 0.9241087 | 0.7343959 | 1.1811877 |
| Phase-1 RCT-229 | 0.8235306 | 0.9708303 | 0.9538933 | 1.0020951 |
| Phase-1 RCT-34 | 0.8465119 | 0.3158725 | 0.3384442 | 0.3596196 |
| Choline kinase | 0.7246327 | 1.5810117 | 1.146868 | 0.9373832 |
| Phase-1 RCT-20 | 0.9511538 | 1.1916534 | 1.1159762 | 1.0540992 |
| Phase-1 RCT-248 | 0.9546592 | 1.1157095 | 1.0476844 | 0.9941281 |

(1) Gene expression data for 6 hour timpoint are presented as mean ratio of treatment/control for all 6 hour predictive genes (Table 20).
(2) Compound and dose abbreviations as in Table 1.
(3) Individual animal number
(4) Liver necrosis classification for compound-dosegroup at 72 h. yes, necrosis observed; no, no necrosis observed
(5) Predictivegene (as in Table 20 and as included in Table 32)

TABLE 35
Expression Data for 24 Hour Timepoint (1)

| | ANIT | ANIT | ANIT | 5-FU | 5-FU | 5-FU | 5-FU |
|---|---|---|---|---|---|---|---|
| Compound-Dose (2) | 15 | 15 | 15 | 13 | 13 | 13 | 50 |
| Animal Number (3) | 1644 | 1645 | 1646 | 1924 | 1925 | 1926 | 1934 |
| Liver Toxicity Necrosis Classification (4) | no | no | no | no | no | no | no |
| Gene Name (5) | | | | | | | |
| Phase-1 RCT-78 | 0.9606432 | 1.0556684 | 1.06886 | 1.0378032 | 1.0988842 | 1.1426469 | 0.9944465 |
| Gamma-actin, cytoplasmic | 1.234655 | 0.8976352 | 1.0834413 | 0.8829685 | 0.9185269 | 1.3651268 | 1.1415585 |
| Matrin F/G | 0.9744384 | 1.1004952 | 0.865002 | 0.8279782 | 1.009797 | 0.8797073 | 1.0522065 |
| Zinc finger protein | 1.1231229 | 1.183528 | 1.0070511 | 1.2828006 | 0.9875436 | 1.0132923 | 1.178386 |
| Cathepsin L, sequence 2 | 0.6737198 | 1.0427825 | 0.7013422 | 0.7277387 | 0.9251564 | 0.7494224 | 0.8220989 |
| Gadd45 | 0.9991312 | 0.8330532 | 0.7687579 | 1.2129412 | 1.4248364 | 1.6599729 | 0.9869085 |
| Phase-1 RCT-144 | 1.0041298 | 1.0179332 | 1.0406581 | 1.1029855 | 1.0888541 | 1.0810028 | 1.1220856 |
| Phase-1 RCT-145 | 1.1349468 | 0.9919431 | 1.0710337 | 1.0738063 | 0.9911743 | 0.9269185 | 1.2875798 |
| Phase-1 RCT-50 | 1.0811491 | 1.1751633 | 1.1560526 | 1.3123729 | 1.1956394 | 1.5409899 | 1.1319916 |
| Phase-1 RCT-92 | 0.7578442 | 0.7398149 | 0.8428189 | 0.9231525 | 0.864412 | 0.8824382 | 0.8727324 |
| Phase-1 RCT-89 | 0.8709707 | 0.9250588 | 0.9641894 | 0.887008 | 1.0033853 | 0.8921654 | 1.0670309 |
| 14-3-3 zeta | 1.2469954 | 1.3013403 | 1.2184939 | 0.8958337 | 0.9736256 | 0.9816141 | 1.0652953 |
| Dynamin-1 (D100) | 0.7341691 | 0.8172326 | 0.8457148 | 1.0695996 | 1.0328438 | 1.0394361 | 0.9577231 |
| Insulin-like growth factor binding protein 1 | 0.8385347 | 1.01372 | 0.7705984 | 0.7539287 | 0.8581647 | 0.7616749 | 1.1499928 |
| L-gulono-gamma-lactone oxidase | 1.035266 | 0.8882917 | 1.2309648 | 0.8881217 | 1.3411688 | 1.0458558 | 1.2213012 |
| Ornithine decarboxylase | 1.6540791 | 1.2079552 | 1.5588424 | 1.315074 | 1.0189186 | 1.0996697 | 1.1511778 |
| PAR interacting protein | 1.0212445 | 1.0472668 | 1.068352 | 1.0239652 | 1.0729176 | 0.9728491 | 1.1765709 |
| Phase-1 RCT-128 | 0.6430531 | 0.7403402 | 0.7893262 | 0.8443767 | 0.7321819 | 0.6534293 | 0.8989345 |
| Phase-1 RCT-180 | 1.3548789 | 1.0220557 | 1.3431201 | 0.8702884 | 1.0285927 | 0.8921868 | 1.284046 |
| Phase-1 RCT-182 | 0.8725214 | 0.9559407 | 0.9858731 | 0.9838916 | 0.7504907 | 0.6978025 | 0.8309025 |
| Phase-1 RCT-207 | 1.123396 | 1.2213591 | 1.0871301 | 1.0950805 | 1.020762 | 0.972094 | 1.8437409 |
| Phase-1 RCT-213 | 1.1572421 | 1.0405387 | 1.0764239 | 1.0014594 | 0.9347457 | 0.8919299 | 1.1561885 |
| Phase-1 RCT-256 | 0.8256394 | 0.756739 | 0.9494309 | 0.9216274 | 1.121024 | 1.0076169 | 1.1053467 |
| Phase-1 RCT-258 | 1.0452662 | 1.0501314 | 0.9829971 | 1.0678148 | 0.9378324 | 0.9536871 | 1.1425503 |
| Phase-1 RCT-264 | 0.6777747 | 0.680146 | 0.7329243 | 0.8753427 | 0.7854723 | 0.967061 | 0.9749348 |
| Phase-1 RCT-271 | 1.0047202 | 0.8360211 | 1.155231 | 0.9694101 | 0.7295578 | 1.1351821 | 0.847829 |
| Phase-1 RCT-288 | 0.7912366 | 0.8019341 | 0.7317085 | 0.8048883 | 0.7987462 | 0.7408214 | 0.9212515 |
| Phase-1 RCT-38 | 0.8092122 | 0.7337164 | 0.9244515 | 0.9042202 | 1.090489 | 1.0620958 | 1.1336697 |
| Phase-1 RCT-39 | 0.9256676 | 0.973714 | 0.9036931 | 0.9808886 | 1.0840786 | 1.2670538 | 0.998117 |
| Phase-1 RCT-68 | 1.082211 | 1.0222166 | 1.0484298 | 1.0217175 | 0.9939265 | 1.0393579 | 1.0164386 |
| Phase-1 RCT-33 | 1.0367855 | 0.8183904 | 1.1262873 | 0.7944518 | 0.9035608 | 0.9077959 | 0.8387765 |
| Phase-1 RCT-36 | 1.0628756 | 0.9383539 | 1.1077182 | 0.9919092 | 1.113472 | 1.0854328 | 0.8942561 |
| 17-beta hydroxysteroid dehydrogenase, | 0.2557269 | 0.4065386 | 0.4871701 | 0.9630411 | 1.3751112 | 0.7476072 | 0.6372146 |
| type 2 | | | | | | | |
| Senescence marker protein-30 | 0.6908734 | 0.761073 | 0.8339542 | 0.7876478 | 0.8078276 | 0.9261982 | 0.8138276 |
| Ribosomal protein S17 | 0.7170245 | 0.6600363 | 0.779886 | 0.6934841 | 0.6917452 | 0.7580662 | 0.9146664 |
| Phase-1 RCT-83 | 0.9355766 | 0.7363179 | 0.6322819 | 0.9126156 | 0.7908109 | 1.0080338 | 0.8607119 |
| Phase-1 RCT-49 | 0.9144406 | 0.9921536 | 1.0273243 | 1.0697739 | 1.1442826 | 1.0990219 | 1.0087235 |
| Phase-1 RCT-48 | 1.0922433 | 1.0387018 | 1.2802345 | 1.3648323 | 1.4315051 | 1.4858004 | 1.4895482 |
| Phase-1 RCT-40 | 0.8138789 | 0.7969445 | 0.7612315 | 0.8024294 | 0.6707104 | 0.7153794 | 0.7943131 |
| Phase-1 RCT-296 | 0.8661627 | 0.7406203 | 0.8728151 | 0.646161 | 0.8784101 | 0.8266384 | 0.6303439 |
| Phase-1 RCT-295 | 0.9571786 | 0.8119676 | 1.0094142 | 0.8923158 | 0.9450644 | 0.9203633 | 1.1216207 |
| Phase-1 RCT-291 | 0.7955568 | 0.8378552 | 0.6871151 | 0.9241879 | 0.9999999 | 0.8549849 | 0.9008082 |
| Phase-1 RCT-270 | 0.6942322 | 0.7076541 | 0.8038533 | 0.9906985 | 0.8756114 | 0.8580422 | 0.9727854 |
| Phase-1 RCT-241 | 1.2400453 | 1.2443434 | 1.2229478 | 1.1740458 | 1.0846578 | 0.9436349 | 1.0631747 |
| Phase-1 RCT-191 | 1.589339 | 0.4355308 | 1.5240867 | 1.0897073 | 1.4454972 | 0.9717998 | 1.1388346 |
| Phase-1 RCT-189 | 0.957172 | 0.9813333 | 1.0627927 | 0.9603199 | 0.9504963 | 1.0071 | 1.1807938 |
| Phase-1 RCT-179 | 0.9294973 | 0.8791354 | 0.8680937 | 0.8467943 | 0.8114437 | 0.7780451 | 1.0111154 |
| Phase-1 RCT-152 | 0.9330756 | 0.7658373 | 0.8526723 | 0.7580634 | 0.7153202 | 0.8203859 | 0.9404895 |
| Phase-1 RCT-123 | 1.0235242 | 0.9633438 | 0.9743514 | 1.0897603 | 1.004225 | 1.1684424 | 0.9645892 |
| Paraoxonase 1 | 0.6920878 | 0.7282934 | 0.6632389 | 0.6540971 | 0.6665652 | 0.7230098 | 0.7127779 |
| Organic anion transporter 3 | 0.7457268 | 0.9852116 | 0.7191772 | 1.0418876 | 0.9527009 | 1.3286465 | 0.7104384 |
| N-hydroxy-2-acetylaminofluorene | 0.7415294 | 0.7526311 | 0.6513983 | 0.7900769 | 0.5953234 | 0.7028629 | 0.638206 |
| sulfotransferase (ST1C1) | | | | | | | |
| Integrin beta1 | 0.9959797 | 0.8837991 | 0.8777278 | 1.1494998 | 1.0974783 | 1.2803507 | 0.9689227 |
| Insulin-like growth factor binding protein 3 | 0.7536389 | 0.8281868 | 0.9423642 | 0.7099455 | 0.8942794 | 0.9921948 | 1.0137434 |
| ID-1 | 1.2034271 | 1.4213678 | 1.3590462 | 0.928004 | 0.91022 | 0.8405138 | 1.1966172 |
| Hepatic lipase | 0.9425939 | 0.6856935 | 1.6810079 | 0.5968204 | 0.723527 | 0.8124986 | 0.6973803 |
| Heme oxygenase | 0.8559955 | 0.8107492 | 0.7697256 | 1.0312041 | 1.0437711 | 1.4447311 | 1.0176487 |
| Equilbrative nitrobenzylthioinosine- | 0.8776665 | 0.8876054 | 0.7660337 | 0.8887283 | 0.7155096 | 0.8258019 | 0.8052473 |
| sensitive nucleoside transporter | | | | | | | |
| Epidermal growth factor | 0.9639346 | 0.9443616 | 0.9327937 | 1.0038493 | 1.0512372 | 1.1399446 | 0.8861233 |
| c-myc | 1.2556394 | 0.9573229 | 0.8723531 | 1.5037686 | 1.3402379 | 1.3130077 | 1.1648532 |
| Carbonic anhydrase III | 0.8394085 | 0.6661759 | 0.7352127 | 0.9730505 | 0.4981356 | 0.4177677 | 0.6849998 |
| Beta-actin | 1.1125833 | 1.0069311 | 1.080633 | 0.6421092 | 0.7735134 | 1.0292419 | 1.0695546 |
| Bax (alpha) | 1.2416624 | 1.1744834 | 1.2701218 | 1.2928851 | 1.418582 | 0.908126 | 1.8201531 |
| Alpha 1 - inhibitor III | 0.6311194 | 0.5582825 | 0.667051 | 0.628073 | 0.567337 | 0.7400826 | 0.8073888 |
| 60S ribosomal protein L6 | 0.9602271 | 0.8815434 | 0.9421652 | 0.7749037 | 0.7103887 | 0.7249129 | 0.9687549 |
| Phase-1 RCT-117 | 1.1647296 | 0.8430245 | 0.795242 | 1.1680948 | 1.2766598 | 1.134663 | 1.5469552 |
| Phase-1 RCT-102 | 0.9053056 | 0.75501 | 0.8611785 | 1.1961399 | 1.6769581 | 1.6983763 | 1.5027566 |
| 3-hydroxyisobutyrate dehydrogenase | 0.7644417 | 0.8099306 | 0.7458267 | 0.8211354 | 0.7821619 | 0.7580239 | 0.8516723 |
| ATPase inhibitor (rat mitochondrial IF1 | 0.802482 | 0.6290388 | 0.6611787 | 0.7644631 | 0.6640281 | 0.699207 | 0.8686532 |
| protein) | | | | | | | |
| Alpha-2-macroglobulin | 0.9666392 | 1.0481755 | 0.7910072 | 0.6375054 | 0.55787 | 0.7578951 | 0.8192324 |
| Phase-1 RCT-137 | 0.8303145 | 0.6784075 | 0.8429167 | 0.8037788 | 0.7698091 | 0.7706092 | 0.912908 |
| Phase-1 RCT 252 | 0.6560146 | 0.8101222 | 0.9351693 | 0.8428133 | 0.9022817 | 0.6775506 | 0.9891068 |
| Phase-1 RCT-65 | 1.1814723 | 1.3648194 | 1.0991114 | 1.1430959 | 1.4789107 | 1.6424018 | 1.2501326 |
| Liver fatty acid binding protein | 0.8225945 | 0.755037 | 0.8761555 | 0.608056 | 1.1298586 | 0.8484246 | 0.8121429 |
| Carbamyl phosphate synthetase I | 0.6134369 | 0.7673738 | 0.8715026 | 0.8482378 | 0.9113016 | 0.6917755 | 1.0281152 |
| c-jun | 1.1744986 | 1.4761358 | 1.1978464 | 1.6867671 | 2.1156347 | 1.9463342 | 1.3228419 |
| Transthyretin | 0.5051306 | 0.5640249 | 0.5808367 | 0.7253085 | 0.6587368 | 0.8447225 | 0.9205499 |
| Aflatoxin B1 aldehyde reductase | 0.9825718 | 0.9719576 | 1.004303 | 0.8631947 | 0.652617 | 0.84073 | 0.9970102 |
| Phase-1 RCT-15 | 1.0476011 | 1.1975862 | 0.9823651 | 1.123427 | 1.1442177 | 1.2747158 | 1.2646374 |
| Insulin-like growth factor I | 0.673672 | 0.5636892 | 0.7470547 | 0.7358452 | 0.9152294 | 1.0104843 | 1.0725784 |
| Sodium/bile acid cotransporter | 0.6770603 | 0.8037157 | 0.5836247 | 0.772383 | 0.7382281 | 0.6717047 | 0.6441643 |
| Organic cation transporter 3 | 0.9872308 | 0.9144946 | 0.9809934 | 1.197376 | 0.957618 | 1.0156827 | 1.0696678 |
| Gadd153 | 1.0656637 | 1.0819016 | 1.1014409 | 1.3004087 | 1.1418839 | 1.2355686 | 1.1323394 |
| Phase-1 RCT-109 | 0.9298786 | 0.9429688 | 1.0163127 | 0.7688204 | 0.9333745 | 0.7531865 | 1.0160506 |
| Phase-1 RCT-88 | 0.8410778 | 0.8999794 | 0.7897615 | 1.0629405 | 0.9164393 | 1.0688851 | 0.8620212 |
| Ubiquitin conjugating enzyme (RAD 6 | 1.0090814 | 0.8631023 | 1.027551 | 0.8936054 | 0.8092048 | 0.9160371 | 0.9142789 |
| homologue) | | | | | | | |
| Glucose-regulated protein 78 | 1.347454 | 0.9249608 | 1.0002779 | 0.7092724 | 0.7203007 | 0.831907 | 1.0513484 |
| Ribosomal protein L13A | 0.9291038 | 0.8786943 | 0.9376202 | 0.7629735 | 0.9454066 | 0.7374412 | 1.0712136 |
| Monocyte chemotactic protein receptor | 1.108431 | 1.0272442 | 1.0699223 | 1.5510839 | 1.1705893 | 1.333799 | 1.2815113 |
| (CCR2) | | | | | | | |
| Fas antigen | 1.2969109 | 1.0780027 | 1.0589168 | 0.9647479 | 0.9796271 | 1.0087534 | 1.2731256 |
| Calpactin I heavy chain | 1.1729448 | 1.1061074 | 1.276587 | 1.1088448 | 1.2847337 | 1.4083927 | 1.0974767 |
| Uncoupling protein 2 | 1.0172572 | 0.932326 | 1.0646602 | 1.0087626 | 1.0422384 | 0.8998179 | 1.0939405 |
| Phase-1 RCT-168 | 0.9633163 | 1.0560569 | 1.3160982 | 0.9709101 | 0.944249 | 1.0057173 | 0.8857675 |
| Phase-1 RCT-154 | 1.1245887 | 0.9558731 | 1.0598948 | 1.0176849 | 1.013118 | 0.9968762 | 2.580363 |
| Superoxide dismutase Mn | 1.1324732 | 1.144354 | 1.0584015 | 0.8849232 | 0.9169562 | 0.9847251 | 1.0782919 |
| Phase-1 RCT-214 | 1.1671687 | 0.9638619 | 1.1359466 | 0.939544 | 0.8672364 | 1 | 1.1081185 |
| Phase-1 RCT-225 | 1.5419785 | 1.5970343 | 1.085522 | 1.2804985 | 1.3645488 | 1.5460782 | 1.1515332 |
| Phase-1 RCT-181 | 1.1259185 | 1.0983542 | 1.0375159 | 0.9117115 | 1.3550928 | 0.8866687 | 0.9872145 |
| Nucleoside diphosphate kinase beta | 1.1012578 | 0.9614247 | 1.2050774 | 0.7670224 | 0.8638704 | 0.7603818 | 0.9720333 |
| isoform | | | | | | | |
| Extracellular-signal-regulated kinase 1 | 0.8534378 | 0.6854154 | 0.7451769 | 1.0550921 | 0.8599343 | 0.8050641 | 0.8898626 |
| {Ribosomal protein L6} | 0.8708381 | 0.7953882 | 0.9023265 | 0.796707 | 0.760862 | 0.8027847 | 1.0141122 |
| Aldehyde dehydrogenase, microsomal | 1.1834677 | 1.1346823 | 1.2817447 | 0.8813519 | 0.9138298 | 0.914008 | 1.080652 |
| p53 | 1.052024 | 1.0942703 | 1.0409782 | 1.0380415 | 0.9094677 | 0.9746472 | 1.1217331 |
| Phase-1 RCT-239 | 1.1428587 | 1.1938224 | 1.1353062 | 1.0183517 | 1.4855719 | 1.4244561 | 1.1483557 |
| Macrophage inflammatory protein-2 alpha | 1.0204061 | 1.3235006 | 0.9874173 | 1.1621082 | 1.1772798 | 1.6081469 | 1.050757 |
| IgE binding protein | 0.9121916 | 0.9083094 | 0.8378812 | 1.0531306 | 1.224349 | 1.3156986 | 1.0154625 |
| Phase-1 RCT-205 | 1.0558898 | 1.1280379 | 1.2269703 | 1.1254572 | 1.3340502 | 1.1391122 | 1.0193665 |
| Melanoma-associated antigen ME491 | 1.0375177 | 1.1374773 | 0.8617728 | 0.92368 | 0.8712776 | 0.7646829 | 1.0675931 |
| Phase-1 RCT-242 | 1.0861074 | 1.1926715 | 1.0917605 | 1.3877577 | 1.1097107 | 1.4220744 | 1.1499097 |
| Enolase alpha | 1.1413764 | 0.7946374 | 1.3107854 | 0.5203328 | 0.5296867 | 0.631955 | 0.5601858 |
| Thymosin beta-10 | 0.9876697 | 0.8630124 | 0.9579362 | 0.8263573 | 0.8805448 | 0.839964 | 1.0476573 |
| Cofilin | 1.0454941 | 0.9625092 | 1.0086012 | 0.8766813 | 0.8280717 | 0.9322122 | 0.979426 |
| Carbonyl reductase | 1.1883663 | 1.2043815 | 1.2608972 | 1.4120765 | 1.0944185 | 1.1135831 | 1.0254887 |
| MAP kinase kinase | 1.0824237 | 1.0124472 | 1.0826418 | 0.8540512 | 0.8864909 | 1.0648104 | 0.8571875 |
| Phase-1 RCT-162 | 0.9305023 | 0.9782598 | 0.9711172 | 0.984266 | 0.9430872 | 0.9813772 | 1.0271157 |
| Phase-1 RCT-72 | 0.9479011 | 0.9927654 | 1.0133151 | 1.202419 | 1.0749412 | 1.5916219 | 1.0047745 |
| Ribosomal protein S8 | 0.772163 | 0.7123377 | 0.8073831 | 0.7435265 | 0.7338651 | 0.7322153 | 0.9177516 |
| Proliferating cell nuclear antigen gene | 0.9405809 | 0.9422681 | 1.0109384 | 1.1720645 | 1.1489159 | 1.1526172 | 0.9886081 |
| Phase-1 RCT-192 | 1.1132158 | 0.9295505 | 0.9692047 | 0.8722476 | 0.8292279 | 0.7715829 | 0.9216096 |
| IkB-a | 0.9691682 | 0.8259872 | 1.1694446 | 0.8077511 | 0.6992489 | 0.8052953 | 0.8794727 |
| Endogenous retroviral sequence, 5′ and | 0.9026088 | 1.1204435 | 0.9442034 | 0.9298239 | 0.9721721 | 0.8393259 | 0.9950646 |
| 3′ LTR | | | | | | | |
| Phase-1 RCT-37 | 0.8961507 | 0.9234294 | 0.9431801 | 1.0185499 | 0.9925371 | 1.2430218 | 1.0544777 |
| Matrix metalloproteinase-1 | 0.6609957 | 0.6991246 | 0.6311756 | 0.7559208 | 0.9048951 | 0.9508686 | 0.835312 |
| Cyclin G | 1.2350241 | 1.2108237 | 1.3522166 | 1.4034588 | 1.0933255 | 1.55363 | 2.3834968 |
| DNA polymerase beta | 0.8664421 | 0.8521425 | 0.9054932 | 0.8683556 | 0.766411 | 0.8994175 | 0.9006014 |
| Phase-1 RCT-127 | 1.0414683 | 0.9647064 | 0.9414419 | 1.0842229 | 1.0088959 | 1.1533852 | 0.9572113 |
| Multidrug resistant protein-2 | 0.8078203 | 1.3479131 | 0.7112554 | 0.9033722 | 0.9381391 | 1.0345547 | 1 |
| Ribosomal protein S9 | 0.9879627 | 0.7876066 | 1.0765914 | 0.7437902 | 0.695443 | 0.7559015 | 0.7701294 |
| Phase-1 RCT-12 | 1.0551718 | 1.036269 | 1.1493162 | 1.0201974 | 1.3652276 | 1.0644789 | 1.1844039 |
| Beta-tubulin, class I | 1.345743 | 0.8566294 | 1.117432 | 0.956182 | 1.4076198 | 1.0629709 | 1.3852527 |
| Argininosuccinate lyase | 1.1025302 | 1.2026094 | 1.0731585 | 0.8724884 | 1.0322863 | 0.7849556 | 1.1032883 |
| Elongation factor-1 alpha | 0.7427184 | 0.8893508 | 0.8544673 | 0.7734079 | 0.787115 | 0.6450148 | 0.7887963 |
| Phase-1 RCT-55 | 1.1534506 | 1.0091838 | 1.1124452 | 1.3796122 | 1.28574 | 2.3383546 | 1.135149 |
| Macrophage inflammatory protein-1 alpha | 1.2219894 | 1.4038032 | 1.3033266 | 1.638195 | 1.2652233 | 1.1574394 | 1.4291952 |
| c-H-ras | 1.2052535 | 1.0465447 | 1.132804 | 0.9831354 | 1.1069292 | 0.9797258 | 1.3339578 |
| Multidrug resistant protein-1 | 0.8336811 | 1.4127753 | 0.7923596 | 0.8892252 | 1.0112568 | 0.8697436 | 1.062138 |
| T-cell cyclophilin | 0.7842779 | 0.7432393 | 0.8265376 | 0.7259359 | 0.6650558 | 0.7443986 | 0.8161539 |
| Phase-1 RCT-185 | 0.7249858 | 0.7483657 | 0.8339102 | 0.9447574 | 0.7118444 | 0.8946133 | 0.8036889 |
| 25-DX | 0.8239855 | 0.9955587 | 0.8126368 | 0.7639309 | 0.7742776 | 0.6594153 | 0.8309735 |
| Ref-1 | 1.0130506 | 0.9716394 | 0.9941383 | 1.5013391 | 1.1508367 | 1.7462074 | 1.2874095 |
| Apolipoprotein CIII | 0.8926475 | 0.875975 | 0.9307526 | 1.0087755 | 0.6908777 | 0.8739698 | 0.973667 |
| NADPH quinone oxidoreductase-1 (DT- | 1.3016022 | 1.1030159 | 1.0716479 | 1.5835896 | 1.316442 | 1.9291593 | 1.6188277 |
| diaphorase) | | | | | | | |
| Pyruvate kinase, muscle | 0.7598411 | 0.9789966 | 0.8111811 | 1.0383656 | 1.3005311 | 1.2687137 | 1.1669873 |
| | 5-FU | 5-FU | APAP | PAP | APAP | AMPB | AMPB |
| Compound-Dose (2) | 50 | 50 | 250 | 250 | 250 | 5 | 5 |
| Animal Number (3) | 1935 | 1936 | 2124 | 2125 | 2126 | 444 | 445 |
| Liver Toxicity Necrosis Classification (4) | no | no | no | no | no | no | no |
| Gene Name (5) | | | | | | | |
| Phase-1 RCT-78 | 0.8264331 | 0.9308587 | 0.9203526 | 0.9098523 | 0.8661597 | 0.9309284 | 1.0404218 |
| Gamma-actin, cytoplasmic | 1.0492682 | 0.8976617 | 0.9818335 | 1.032798 | 1.0409787 | 0.5889278 | 0.6080275 |
| Matrin F/G | 1.06837 | 1.2039248 | 1.2915198 | 1.3863662 | 1.274342 | 1.0725853 | 0.9228077 |
| Zinc finger protein | 1.0754057 | 1.1371926 | 1.0537326 | 1.0233865 | 1.1575507 | 1.0295161 | 0.9915832 |
| Cathepsin L, sequence 2 | 0.8208284 | 0.8365451 | 0.8534969 | 0.8992265 | 0.80499 | 1.3029445 | 1.0383123 |
| Gadd45 | 1.3720591 | 1.0139263 | 1.0286342 | 0.9542578 | 0.9179477 | 1.1298801 | 1.0124631 |
| Phase-1 RCT-144 | 1.0345267 | 1.2151896 | 0.9828554 | 0.9194126 | 0.9596442 | 0.769776 | 0.8926844 |
| Phase-1 RCT-145 | 1.1361277 | 1.2261806 | 1.0253922 | 0.9850923 | 0.9777477 | 0.9779645 | 1.0004281 |
| Phase-1 RCT-50 | 1.1666703 | 1.1848195 | 1.0551788 | 1.089631 | 1.103832 | 0.8877311 | 0.8995088 |
| Phase-1 RCT-92 | 0.7225307 | 0.830805 | 0.659712 | 0.871622 | 0.8555691 | 1.0753319 | 0.990141 |
| Phase-1 RCT-89 | 0.9204272 | 1.0734688 | 0.9725134 | 0.9925594 | 0.8346179 | 1.1420949 | 0.8569865 |
| 14-3-3 zeta | 1.1281042 | 1.1210417 | 1.3165957 | 1.2755212 | 1.0667768 | 0.8864849 | 1.001191 |
| Dynamin-1 (D100) | 0.9478024 | 1.0657479 | 1.0713295 | 0.9927657 | 0.9898405 | 1.169634 | 1.1217867 |
| Insulin-like growth factor binding protein 1 | 1.1458775 | 0.9828581 | 1.1296431 | 1.1723659 | 1.176557 | 1.2116332 | 1.193881 |
| L-gulono-gamma-lactone oxidase | 1.1252539 | 0.9716736 | 1.0050704 | 1.296565 | 1.0625685 | 0.8201332 | 0.6984235 |
| Ornithine decarboxylase | 1.426834 | 1.3840773 | 1.9487185 | 1.8499516 | 1.7758799 | 0.8358308 | 0.8981549 |
| PAR interacting protein | 1.0820519 | 1.1654148 | 0.9788213 | 0.9999226 | 0.8972472 | 0.8656605 | 0.9585217 |
| Phase-1 RCT-128 | 0.6975887 | 1.0062159 | 0.9543832 | 0.9381317 | 0.8103129 | 1.2472311 | 1.1158968 |
| Phase-1 RCT-180 | 1.0138234 | 0.8948993 | 1.0824504 | 1.087474 | 1.0474179 | 1.0517182 | 1.0971311 |
| Phase-1 RCT-182 | 0.7459426 | 0.9820361 | 0.9656007 | 0.7314658 | 0.771692 | 1.0325558 | 0.8200564 |
| Phase-1 RCT-207 | 1.9543331 | 2.3668187 | 1.1815271 | 1.1913304 | 1.1588718 | 0.8538696 | 1.0764338 |
| Phase-1 RCT-213 | 1.1128368 | 1.1354781 | 1.1243172 | 1.0841323 | 1.0840886 | 0.932004 | 1.0209827 |
| Phase-1 RCT-256 | 1.0199891 | 0.9926932 | 1.0094044 | 1.1078622 | 1.0561546 | 1.2516383 | 0.9810751 |
| Phase-1 RCT-258 | 0.9913303 | 1.0752094 | 1.0369308 | 0.9895516 | 1.0010142 | 0.847468 | 0.9513122 |
| Phase-1 RCT-264 | 0.6272578 | 0.7679091 | 0.7485089 | 0.8746949 | 0.6019432 | 1.2118464 | 1.2089987 |
| Phase-1 RCT-271 | 0.7969024 | 0.8014067 | 0.8345232 | 1.0360738 | 0.8981077 | 1.0113114 | 1.2025958 |
| Phase-1 RCT-288 | 0.7481297 | 0.8201597 | 0.7995217 | 0.8382061 | 0.7633603 | 1.1391088 | 1.0509446 |
| Phase-1 RCT-38 | 1.0868694 | 0.9566376 | 1.0207416 | 1.1934133 | 1.0345111 | 1.2936591 | 1.0296395 |
| Phase-1 RCT-39 | 1.1593198 | 1.0193778 | 1.1706781 | 1.0454218 | 0.9506837 | 0.900013 | 0.9563121 |
| Phase-1 RCT-68 | 0.9998055 | 0.9702952 | 1.2556094 | 1.3145684 | 1.1844678 | 1.0051438 | 1.0124631 |
| Phase-1 RCT-33 | 0.8504484 | 0.8223167 | 0.9478408 | 1.072374 | 0.8960295 | 1.0881449 | 0.8494526 |
| Phase-1 RCT-36 | 0.9455909 | 0.8934879 | 1.0209336 | 1.096263 | 0.9025034 | 0.9375606 | 0.8446715 |
| 17-beta hydroxysteroid dehydrogenase, | 1.1259855 | 1.0530969 | 0.6602028 | 0.51389 | 0.3486026 | 1.0215769 | 0.9373793 |
| type 2 | | | | | | | |
| Senescence marker protein-30 | 0.7092474 | 0.7384215 | 0.7951443 | 0.8592866 | 0.732002 | 1.3176931 | 0.9055896 |
| Ribosomal protein S17 | 0.7997774 | 0.7929199 | 0.8610807 | 0.9940348 | 0.8382898 | 1.3360245 | 1.3346441 |
| Phase-1 RCT-83 | 0.813229 | 0.7595651 | 0.7245517 | 0.9334855 | 0.7087203 | 0.9741879 | 1.0609227 |
| Phase-1 RCT-49 | 1.0197467 | 1.0847362 | 0.9250758 | 0.9791626 | 0.885897 | 0.7539586 | 0.9205005 |
| Phase-1 RCT-48 | 0.9047989 | 1.0702626 | 1.358267 | 1.1895674 | 1.0349641 | 1.3473293 | 1.2138255 |
| Phase-1 RCT-40 | 0.6568417 | 0.7594329 | 0.8548482 | 0.8847627 | 0.7293569 | 1.0289422 | 1.0779145 |
| Phase-1 RCT-296 | 0.5118952 | 0.5849822 | 0.719744 | 0.9032642 | 0.7253511 | 0.9592091 | 0.7299408 |
| Phase-1 RCT-295 | 1.0286902 | 1.0815953 | 1.1734005 | 1.2967525 | 1.0849984 | 1.1969392 | 1.1117816 |
| Phase-1 RCT-291 | 0.8065127 | 0.777191 | 1.0751426 | 1.034983 | 1.0676949 | 0.8186351 | 0.8476734 |
| Phase-1 RCT-270 | 0.8435893 | 0.9071513 | 0.9524308 | 0.8710589 | 0.859674 | 1.0831913 | 0.8703755 |
| Phase-1 RCT-241 | 1.1138122 | 0.9605852 | 1.0045639 | 0.9200067 | 1.153423 | 0.9170438 | 0.8902995 |
| Phase-1 RCT-191 | 0.9875454 | 1.1399181 | 1.2239399 | 1.1713823 | 1.0227512 | 0.7916213 | 0.944196 |
| Phase-1 RCT-189 | 1.0361978 | 1.186117 | 0.9110143 | 0.8260672 | 0.7520856 | 1.1312664 | 1.1596029 |
| Phase-1 RCT-179 | 0.831642 | 0.9108703 | 0.9625362 | 0.9199495 | 0.8235564 | 1.2064903 | 1.2141274 |
| Phase-1 RCT-152 | 0.8495509 | 0.9490785 | 0.7835439 | 0.8992117 | 0.7232087 | 1.2008587 | 1.0749402 |
| Phase-1 RCT-123 | 0.9104691 | 1.0329788 | 1.0294486 | 1.0414743 | 1.094004 | 0.948637 | 0.9799223 |
| Paraoxonase 1 | 0.6636318 | 0.6696453 | 0.5639626 | 0.782172 | 0.6893359 | 1.0735302 | 0.9749345 |
| Organic anion transporter 3 | 1.3038136 | 1.1778989 | 1.2156521 | 1.1630347 | 1.1539832 | 1.1097461 | 0.9605166 |
| N-hydroxy-2-acetylaminofluorene | 0.5931203 | 0.5926167 | 0.780278 | 0.9368148 | 0.8355691 | 0.9899004 | 1.0606474 |
| sulfotransferase (ST1C1) | | | | | | | |
| Integrin beta1 | 1.295391 | 1.2332006 | 1.2903486 | 1.2740508 | 1.3898692 | 1.2685422 | 1.0535703 |
| Insulin-like growth factor binding protein 3 | 0.9752592 | 1.0442053 | 0.774142 | 0.8645647 | 0.6147066 | 1.2881382 | 0.9133946 |
| ID-1 | 1.0789943 | 1.215396 | 1.0370384 | 1.0159979 | 0.9002337 | 0.9515531 | 0.9025709 |
| Hepatic lipase | 0.580999 | 0.6124978 | 0.8202561 | 0.8267142 | 0.9853741 | 0.9025518 | 0.7897592 |
| Heme oxygenase | 0.9735902 | 0.81857 | 1.1482412 | 1.2556607 | 0.9316275 | 1.325053 | 0.8858699 |
| Equilbrative nitrobenzylthioinosine- | 0.8245753 | 0.6647123 | 0.8785653 | 1.0180075 | 0.9749414 | 0.9590571 | 0.9896835 |
| sensitive nucleoside transporter | | | | | | | |
| Epidermal growth factor | 0.9806534 | 0.944624 | 0.8196384 | 0.9033831 | 0.7848426 | 1.516955 | 1.2809311 |
| c-myc | 1.3555838 | 1.1519816 | 1.269491 | 1.0737239 | 1.2187063 | 1.0842137 | 1.0022017 |
| Carbonic anhydrase III | 0.5937173 | 0.938281 | 0.9307031 | 1.0554777 | 0.5985045 | 1.0996143 | 1.4797466 |
| Beta-actin | 0.967212 | 0.919635 | 3.0672295 | 2.4768662 | 2.7791693 | 0.874977 | 0.7595192 |
| Bax (alpha) | 1.6742516 | 2.4587035 | 1.295488 | 1.1407483 | 1.307053 | 0.9552065 | 0.9472449 |
| Alpha 1 - inhibitor III | 0.6584054 | 0.8384187 | 0.4391303 | 0.6929265 | 0.6857092 | 0.5854951 | 0.9438677 |
| 60S ribosomal protein L6 | 0.8730169 | 0.890568 | 0.8307589 | 0.8993789 | 0.7420199 | 1.0680226 | 1.0149685 |
| Phase-1 RCT-117 | 1.1515244 | 0.931738 | 0.5392493 | 0.984865 | 0.907024 | 0.7383441 | 1.137953 |
| Phase-1 RCT-102 | 0.9443657 | 1.0775381 | 0.6762655 | 0.762919 | 0.5259215 | 0.9226028 | 0.823916 |
| 3-hydroxyisobutyrate dehydrogenase | 0.7781804 | 0.8586909 | 1.0065533 | 0.9518525 | 0.8933026 | 1.1824843 | 1.1520633 |
| ATPase inhibitor (rat mitochondrial IF1 | 0.719345 | 0.8293529 | 0.6792845 | 0.6941613 | 0.5084229 | 1.4203913 | 1.2955924 |
| protein) | | | | | | | |
| Alpha-2-macroglobulin | 0.6865777 | 0.8395349 | 0.727021 | 0.7683781 | 0.9628826 | 0.6194958 | 1.3751622 |
| Phase-1 RCT-137 | 0.7666324 | 0.9301438 | 0.7139007 | 0.7273785 | 0.6056705 | 1.3410574 | 1.5034796 |
| Phase-1 RCT 252 | 0.816385 | 0.8081465 | 0.8312343 | 0.80571 | 0.6828082 | 0.9844119 | 0.8206223 |
| Phase-1 RCT-65 | 1.378758 | 1.3046662 | 1.5337906 | 1.5226179 | 1.3918132 | 0.949553 | 1.0112531 |
| Liver fatty acid binding protein | 0.6958506 | 0.6339875 | 0.7487941 | 0.9535539 | 0.633476 | 1.3605846 | 1.2572317 |
| Carbamyl phosphate synthetase I | 0.8562396 | 0.8177047 | 0.8605284 | 0.8054114 | 0.7509247 | 0.9854083 | 0.8224081 |
| c-jun | 1.8350563 | 1.4393637 | 0.9835252 | 1.0151979 | 0.9719749 | 1.0713656 | 1.0493238 |
| Transthyretin | 0.5872653 | 0.6551925 | 0.6613085 | 0.7890704 | 0.5748965 | 1.0544994 | 1.0695916 |
| Aflatoxin B1 aldehyde reductase | 0.9282437 | 2.0246065 | 0.722496 | 0.9845435 | 0.8905217 | 0.9831285 | 1.0293256 |
| Phase-1 RCT-15 | 1.2709618 | 1.1686368 | 1.4327178 | 1.4164921 | 1.4717025 | 0.9947144 | 1.1576836 |
| Insulin-like growth factor I | 0.7755994 | 0.9910879 | 0.796438 | 0.7801899 | 0.6827156 | 1.2177734 | 0.9285826 |
| Sodium/bile acid cotransporter | 0.6426185 | 0.7971108 | 0.5944278 | 1.0647414 | 0.6077975 | 1.1732075 | 0.9933712 |
| Organic cation transporter 3 | 1.1952068 | 1.0582124 | 0.7761408 | 0.856324 | 0.8057378 | 1.1218594 | 1.0644948 |
| Gadd153 | 1.3915831 | 1.2783103 | 1.2222211 | 1.0213656 | 1.2682419 | 1.0942502 | 1.0674458 |
| Phase-1 RCT-109 | 0.9374391 | 0.9456841 | 1.1642103 | 1.2189075 | 1.030518 | 1.0794307 | 1.0029254 |
| Phase-1 RCT-88 | 0.7713925 | 0.9519671 | 0.6626439 | 0.8254275 | 0.8373817 | 0.9800136 | 1.04734 |
| Ubiquitin conjugating enzyme (RAD 6 | 0.8923608 | 0.9667737 | 0.8466024 | 0.9069464 | 0.814561 | 1.131639 | 1.1849248 |
| homologue) | | | | | | | |
| Glucose-regulated protein 78 | 1.1012429 | 0.8680978 | 1.4693646 | 1.0288177 | 1.1946875 | 1.3228414 | 1.0660775 |
| Ribosomal protein L13A | 0.9976696 | 0.9033369 | 1.3120813 | 1.2298342 | 1.1284492 | 1.2836893 | 1.2161244 |
| Monocyte chemotactic protein receptor | 1.5947446 | 1.5423346 | 0.9190399 | 0.9828574 | 1.0863031 | 0.9213958 | 0.986389 |
| (CCR2) | | | | | | | |
| Fas antigen | 1.1208655 | 1.1012806 | 1.5384216 | 1.3954457 | 1.6575236 | 1.0451683 | 1.0708269 |
| Calpactin I heavy chain | 1.2257118 | 1.0623697 | 1.3189057 | 1.3670574 | 1.176196 | 1.0492951 | 1.0503352 |
| Uncoupling protein 2 | 1.2260782 | 1.0403274 | 1.156883 | 1.1235359 | 1.1682943 | 1.1405665 | 1.2100816 |
| Phase-1 RCT-168 | 0.8356137 | 0.7324105 | 0.8088246 | 0.8856127 | 0.7707364 | 0.8763399 | 0.8311132 |
| Phase-1 RCT-154 | 2.2507923 | 2.525899 | 0.9833191 | 1.0331408 | 0.9781032 | 1.1033485 | 1.0670265 |
| Superoxide dismutase Mn | 1.0041932 | 1.0214748 | 1.1370399 | 1.1964532 | 1.02008 | 1.7116846 | 1.2457026 |
| Phase-1 RCT-214 | 0.846329 | 0.6486018 | 1.081729 | 1.2471573 | 1.2982978 | 0.9627687 | 1.0128975 |
| Phase-1 RCT-225 | 1.4604083 | 1.9018307 | 1.5824646 | 1.6279248 | 0.915002 | 1.0538584 | 0.9361214 |
| Phase-1 RCT-181 | 1.0954368 | 1.0280274 | 1.0151833 | 1.0094173 | 0.9226298 | 0.9249831 | 0.9039672 |
| Nucleoside diphosphate kinase beta | 0.7992253 | 0.8638516 | 1.0197171 | 1.2295636 | 0.8894069 | 1.1502217 | 1.3752702 |
| isoform | | | | | | | |
| Extracellular-signal-regulated kinase 1 | 1.1070317 | 1.075846 | 1.0540497 | 1.1447276 | 0.7444317 | 0.9680275 | 1.3552467 |
| {Ribosomal protein L6} | 0.7890627 | 0.8916913 | 0.8670989 | 0.9519953 | 0.8320139 | 1.2267033 | 1.0718535 |
| Aldehyde dehydrogenase, microsomal | 0.9089831 | 0.8768838 | 0.8556882 | 0.9467101 | 0.8487862 | 1.1384561 | 1.067368 |
| p53 | 1.2351357 | 1.1178073 | 0.9108433 | 0.8760809 | 1.0271506 | 1.1171085 | 1.0065411 |
| Phase-1 RCT-239 | 1.1877072 | 1.2254268 | 1.2677562 | 1.342256 | 1.3076787 | 0.7406275 | 0.8298853 |
| Macrophage inflammatory protein-2 alpha | 1.3891182 | 1.4428144 | 1.1066924 | 1.0778382 | 1.208523 | 1.2132936 | 1.1950506 |
| IgE binding protein | 1.0802361 | 1.0328445 | 1.0943258 | 1.096537 | 1.0439243 | 1.0789162 | 0.9736975 |
| Phase-1 RCT-205 | 0.9727485 | 1.1119434 | 0.8788992 | 0.9332462 | 0.9377763 | 0.7945797 | 0.9737628 |
| Melanoma-associated antigen ME491 | 0.9731291 | 0.9615104 | 0.9180655 | 0.9607673 | 0.8656814 | 0.8593465 | 0.972976 |
| Phase-1 RCT-242 | 1.2405444 | 1.4084667 | 1.1248877 | 1.1911055 | 1.20148 | 0.7011897 | 0.9541348 |
| Enolase alpha | 0.4232743 | 0.7050132 | 0.9049337 | 0.9241083 | 1.0508192 | 1.2150401 | 1.5965631 |
| Thymosin beta-10 | 1.0547793 | 0.9079984 | 1.0283252 | 1.0723436 | 1.0575863 | 1.3530982 | 1.2984315 |
| Cofilin | 0.835503 | 0.9433985 | 0.8527123 | 0.930896 | 0.913214 | 1.0442383 | 1.1501076 |
| Carbonyl reductase | 1.421994 | 1.1156356 | 1.120373 | 0.9737988 | 1.2524985 | 0.8795914 | 1.0405794 |
| MAP kinase kinase | 1.0985358 | 0.9053413 | 0.9758264 | 0.9617103 | 0.952728 | 0.9804574 | 0.9181999 |
| Phase-1 RCT-162 | 0.8819966 | 0.9545043 | 0.9232208 | 0.9957115 | 0.8568474 | 1.187671 | 1.1679085 |
| Phase-1 RCT-72 | 1.3980849 | 1.0493258 | 1.1065431 | 1.1870025 | 1.0982953 | 0.8266573 | 0.9001768 |
| Ribosomal protein S8 | 0.8158994 | 0.8492166 | 0.8139034 | 0.92109 | 0.7418779 | 1.3837492 | 1.2226822 |
| Proliferating cell nuclear antigen gene | 1.2565892 | 1.0492861 | 0.8932141 | 0.9160172 | 0.9974282 | 1.0243479 | 1.0754528 |
| Phase-1 RCT-192 | 0.8232579 | 0.875704 | 1.1178906 | 1.1181488 | 0.8020556 | 1.2273967 | 1.2119337 |
| IkB-a | 0.8224846 | 0.8824672 | 0.8367836 | 0.9914149 | 0.9959012 | 0.9629551 | 1.0242637 |
| Endogenous retroviral sequence, 5′ and | 0.9160741 | 1.2891484 | 1.5841322 | 1.2960659 | 0.9523855 | 0.9994521 | 0.8615626 |
| 3′ LTR | | | | | | | |
| Phase-1 RCT-37 | 0.9756728 | 0.9541688 | 0.947503 | 1.0052108 | 0.94906 | 1.0808233 | 1.0024878 |
| Matrix metalloproteinase-1 | 0.8100137 | 0.8333569 | 1.1158096 | 1.1268581 | 1.1223301 | 1.0812477 | 1.2793016 |
| Cyclin G | 1.87699 | 3.7271981 | 1.6658889 | 1.0566213 | 1.099879 | 1.0808889 | 0.9943242 |
| DNA polymerase beta | 0.9189502 | 0.9204193 | 0.7988831 | 0.9703308 | 0.8867658 | 1.124575 | 1.1490761 |
| Phase-1 RCT-127 | 1.030932 | 0.9933307 | 0.786047 | 1.0096066 | 1.008742 | 0.9139869 | 1.0123742 |
| Multidrug resistant protein-2 | 1.6298143 | 1.9177527 | 1.0098183 | 1.0122682 | 1.1600221 | 1.1390125 | 1.000016 |
| Ribosomal protein S9 | 0.6570891 | 0.7727515 | 1.0753275 | 0.9525019 | 0.9957287 | 1.347017 | 1.4554441 |
| Phase-1 RCT-12 | 1.1514064 | 1.256103 | 1.1918987 | 1.1572877 | 0.8945466 | 0.8727618 | 1.0715595 |
| Beta-tubulin, class I | 1.2803719 | 1.5404369 | 1.1870043 | 1.374863 | 1.1042604 | 0.9138164 | 1.1618305 |
| Argininosuccinate lyase | 0.8042617 | 1.0600874 | 1.1295489 | 1.135157 | 0.8669316 | 1.0717185 | 1.1093702 |
| Elongation factor-1 alpha | 0.7970261 | 0.8564949 | 0.8353572 | 0.8698468 | 0.9081252 | 1.0106614 | 0.9360194 |
| Phase-1 RCT-55 | 1.5350391 | 1.0821584 | 1.1189781 | 1.1290827 | 1.3078299 | 0.7137425 | 0.7682591 |
| Macrophage inflammatory protein-1 alpha | 1.7488328 | 2.1291108 | 1.2596482 | 1.2336874 | 1.2911267 | 0.7908793 | 1.0198528 |
| c-H-ras | 1.1885431 | 1.2420834 | 1.0740927 | 0.9608217 | 1.1733519 | 0.9286088 | 1.0100199 |
| Multidrug resistant protein-1 | 1.550949 | 2.2784832 | 1.3464178 | 1.2392595 | 1.3891298 | 1.2192514 | 1.2031754 |
| T-cell cyclophilin | 0.7321016 | 0.758857 | 0.7517646 | 0.9529529 | 0.7786428 | 1.1996833 | 1.1822731 |
| Phase-1 RCT-185 | 0.6675405 | 0.8435878 | 0.7561237 | 0.7464 | 0.7109049 | 0.9765083 | 1.0357654 |
| 25-DX | 0.7430027 | 0.9571102 | 0.8511507 | 0.9995099 | 0.8263345 | 1.2966344 | 1.0659928 |
| Ref-1 | 1.6648233 | 1.474214 | 0.9331438 | 1.1119254 | 1.0734626 | 1.0426857 | 1.0633229 |
| Apolipoprotein CIII | 0.7729409 | 0.9612901 | 0.919155 | 0.9429518 | 1.0053529 | 1.0392569 | 1.2722049 |
| NADPH quinone oxidoreductase-1 (DT- | 1.5852265 | 1.8408571 | 0.8544312 | 1.1228857 | 0.9491565 | 1.08327 | 1.1104908 |
| diaphorase) | | | | | | | |
| Pyruvate kinase, muscle | 1.2623621 | 1.049505 | 1.2455558 | 1.1347631 | 1.2077923 | 1.0268922 | 0.9320446 |
| | AMPB | AMPB | AMPB | AMPB | AZA | AZA | AZA |
| Compound-Dose (2) | 5 | 20 | 20 | 20 | 50 | 50 | 50 |
| Animal Number (3) | 446 | 454 | 455 | 456 | 1824 | 1825 | 1826 |
| Liver Toxicity Necrosis Classification (4) | no | no | no | no | no | no | no |
| Gene Name (5) | | | | | | | |
| Phase-1 RCT-78 | 0.9070634 | 0.9480701 | 0.7926477 | 0.847036 | 0.9298717 | 0.9558975 | 1.0712345 |
| Gamma-actin, cytoplasmic | 0.5821251 | 0.7871822 | 0.7093914 | 0.775439 | 0.7523052 | 0.7578945 | 0.8713903 |
| Matrin F/G | 0.886426 | 0.9947434 | 1.0442108 | 1.0063747 | 1.4353818 | 1.6712931 | 1.4580698 |
| Zinc finger protein | 1.0740062 | 0.9079414 | 0.9745604 | 1.0340985 | 1.0890789 | 1.1491843 | 0.9533254 |
| Cathepsin L, sequence 2 | 0.9416942 | 1.1328342 | 1.1602902 | 1.1673063 | 1.1145176 | 1.1013969 | 1.7933372 |
| Gadd45 | 0.9549831 | 0.9350914 | 0.9430864 | 1.0063 | 1.0348563 | 0.829614 | 0.8231778 |
| Phase-1 RCT-144 | 1.0955491 | 0.8869591 | 0.8532358 | 0.8862108 | 0.9918908 | 0.8365974 | 0.9387686 |
| Phase-1 RCT-145 | 1.0577755 | 1.0824317 | 0.9454147 | 0.9915661 | 1.0604832 | 1.0167998 | 1.1809617 |
| Phase-1 RCT-50 | 1.0817329 | 1.025011 | 0.9490913 | 0.9360425 | 1.44215 | 1.0917292 | 1.2234671 |
| Phase-1 RCT-92 | 0.8397044 | 0.9191499 | 1.2321131 | 0.8645302 | 0.752016 | 0.9808349 | 0.6368073 |
| Phase-1 RCT-89 | 0.9314401 | 1.177015 | 1.0667709 | 1.0850333 | 0.8057168 | 1.169088 | 1.0576782 |
| 14-3-3 zeta | 1.0588349 | 0.991496 | 1.0395159 | 1.0589399 | 1.1267045 | 0.9881783 | 0.9280008 |
| Dynamin-1 (D100) | 1.0587527 | 1.0252057 | 0.8899 | 0.9371175 | 1.021319 | 1.1168324 | 0.8928621 |
| Insulin-like growth factor binding protein 1 | 0.9362947 | 1.1707132 | 1.0164272 | 0.9082892 | 0.9060388 | 1.1553967 | 1.0494683 |
| L-gulono-gamma-lactone oxidase | 0.6687783 | 0.7165173 | 0.8466016 | 0.7172374 | 0.5343522 | 0.6695687 | 0.4201693 |
| Ornithine decarboxylase | 0.8963891 | 0.9529114 | 0.9510727 | 1.0162787 | 1.4556915 | 1.2827682 | 1.4496058 |
| PAR interacting protein | 1.0811801 | 0.9411619 | 0.9824935 | 0.9503574 | 1.1913778 | 1.0059475 | 1.116744 |
| Phase-1 RCT-128 | 0.9199231 | 1.0481011 | 0.8305293 | 0.8376861 | 0.896084 | 1.3919032 | 0.8506981 |
| Phase-1 RCT-180 | 1.0686349 | 1.0443985 | 1.1445392 | 1.136211 | 0.928463 | 0.899411 | 0.7842507 |
| Phase-1 RCT-182 | 0.7265104 | 0.7444952 | 0.8206751 | 1.0056177 | 0.9856907 | 1.1356047 | 1.2026912 |
| Phase-1 RCT-207 | 0.7820309 | 0.9439779 | 0.9122205 | 1.0076088 | 1.2666013 | 1.20434 | 1.0074526 |
| Phase-1 RCT-213 | 1.0820234 | 0.9968559 | 1.0356328 | 1.1032516 | 0.9922274 | 1.1935297 | 1.0588398 |
| Phase-1 RCT-256 | 0.8939296 | 1.1084479 | 0.9758979 | 0.8989366 | 0.9687098 | 1.0142882 | 0.8772666 |
| Phase-1 RCT-258 | 1.0999037 | 0.919677 | 0.8904463 | 0.9717339 | 1.0101322 | 1.0905071 | 1.0150908 |
| Phase-1 RCT-264 | 0.8343306 | 1.0359182 | 0.9829549 | 0.7664022 | 0.9576483 | 0.9407848 | 0.9120599 |
| Phase-1 RCT-271 | 1.1246231 | 1.0966185 | 0.9050379 | 0.8800845 | 1.285325 | 1.6049888 | 0.7109682 |
| Phase-1 RCT-288 | 1.0585071 | 1.0273583 | 0.9688564 | 1.0499824 | 0.629191 | 1.0559647 | 0.5562486 |
| Phase-1 RCT-38 | 0.9184311 | 1.1572365 | 0.9668758 | 0.8595974 | 0.834435 | 0.8915342 | 0.8894174 |
| Phase-1 RCT-39 | 0.9086675 | 0.9210828 | 1.1095364 | 1.015182 | 0.8732016 | 0.7374082 | 1.0035868 |
| Phase-1 RCT-68 | 1.0477191 | 1.2328756 | 1.2969031 | 1.0559149 | 1.193551 | 1.1469512 | 1.3091723 |
| Phase-1 RCT-33 | 0.8260297 | 1.0429465 | 1.0128429 | 0.8738492 | 0.6952157 | 0.7285301 | 0.702221 |
| Phase-1 RCT-36 | 0.9841526 | 0.9435421 | 0.9088171 | 0.9216593 | 0.9419789 | 0.8490725 | 0.8333413 |
| 17-beta hydroxysteroid dehydrogenase, | 1.0147817 | 0.7625936 | 0.9838565 | 0.8993297 | 0.7610686 | 0.5378354 | 0.8026193 |
| type 2 | | | | | | | |
| Senescence marker protein-30 | 0.83561 | 1.0821384 | 0.9235263 | 1.1342849 | 0.2245676 | 0.3781015 | 0.3116318 |
| Ribosomal protein S17 | 1.0631652 | 1.7532421 | 1.3206248 | 1.246554 | 1.371442 | 1.4234012 | 2.4839084 |
| Phase-1 RCT-83 | 1.0174524 | 1.2055792 | 1.150247 | 1.145355 | 1.293814 | 0.9844557 | 1.5269971 |
| Phase-1 RCT-49 | 1.2418224 | 0.8990266 | 0.9240159 | 0.9285115 | 1.0464156 | 0.9035581 | 0.8942486 |
| Phase-1 RCT-48 | 1.2273251 | 1.0794547 | 1.0750862 | 1.0609623 | 0.9523583 | 1.2105967 | 0.8602574 |
| Phase-1 RCT-40 | 0.8713576 | 1.2471164 | 1.0392296 | 1.1495807 | 1.0016414 | 1.4191339 | 1.525431¹ |
| Phase-1 RCT-296 | 0.6731812 | 0.5559927 | 1.1618317 | 0.7357143 | 0.9135502 | 0.6976052 | 0.7169617 |
| Phase-1 RCT-295 | 1.0465214 | 1.3802288 | 1.1799 | 1.0115302 | 1.3204356 | 1.2635342 | 1.6548408 |
| Phase-1 RCT-291 | 0.8079996 | 0.7205252 | 0.8697816 | 0.8761358 | 1.015373 | 0.9752554 | 0.9942117 |
| Phase-1 RCT-270 | 0.9101095 | 1.1034979 | 1.0178612 | 1.1249268 | 0.8001629 | 1.2150624 | 0.6599876 |
| Phase-1 RCT-241 | 0.9545099 | 0.8622955 | 0.9135238 | 1.1912299 | 0.960994 | 0.9975827 | 1.1486952 |
| Phase-1 RCT-191 | 1.0833651 | 0.9738737 | 1.061395 | 0.9773905 | 1.577858 | 1.4074147 | 1.3583895 |
| Phase-1 RCT-189 | 1.0450487 | 1.3901923 | 1.0421152 | 1.2881461 | 0.6534645 | 0.8842895 | 0.6498718 |
| Phase-1 RCT-179 | 1.1106943 | 1.2614957 | 1.0967388 | 1.091425 | 1.0577947 | 1.0796807 | 0.9477445 |
| Phase-1 RCT-152 | 0.8369955 | 1.6744584 | 1.1059141 | 1.0406197 | 1.2117712 | 1.1903574 | 2.1739435 |
| Phase-1 RCT-123 | 1.1886034 | 0.9520908 | 1.0198792 | 1.0053626 | 1.0742905 | 0.9976288 | 1.0744553 |
| Paraoxonase 1 | 0.7153009 | 0.5801933 | 0.8143748 | 0.7854477 | 0.4736557 | 0.7183936 | 0.6899841 |
| Organic anion transporter 3 | 0.9488077 | 0.8956594 | 0.9092578 | 0.8788957 | 0.5053284 | 0.7804855 | 0.751599 |
| N-hydroxy-2-acetylaminofluorene | 0.8711633 | 1.3331565 | 0.9139637 | 0.9922501 | 0.5797132 | 0.9697285 | 1.162436 |
| sulfotransferase (ST1C1) | | | | | | | |
| Integrin beta1 | 1.1629019 | 1.2545536 | 1.0665374 | 1.0598626 | 0.8244765 | 0.9259602 | 0.962172 |
| Insulin-like growth factor binding protein 3 | 0.831884 | 1.1556784 | 1.1821109 | 1.1669447 | 0.7135422 | 0.9059209 | 0.9254208 |
| ID-1 | 1.1944456 | 1.0437014 | 0.9694996 | 0.9944603 | 1.0585251 | 1.1374294 | 0.8796557 |
| Hepatic lipase | 0.832729 | 0.5824089 | 0.7604451 | 0.7242364 | 0.4572208 | 0.7791846 | 0.6205136 |
| Heme oxygenase | 1.0590959 | 1.2553945 | 1.2038336 | 1.5259259 | 1.2260115 | 0.9720785 | 1.3517003 |
| Equilbrative nitrobenzylthioinosine- | 0.713909 | 1.1697053 | 0.9976289 | 0.9830484 | 0.7818351 | 0.9292593 | 1.1171777 |
| sensitive nucleoside transporter | | | | | | | |
| Epidermal growth factor | 1.0176045 | 1.7017363 | 1.4153259 | 1.3683574 | 0.9635668 | 1.0265025 | 1.0941824 |
| c-myc | 1.2002887 | 0.9308433 | 0.8984804 | 0.9068002 | 1.2421045 | 1.1196234 | 0.9720898 |
| Carbonic anhydrase III | 0.8531348 | 2.4647226 | 0.9153584 | 0.9614183 | 0.4754552 | 1.3172245 | 0.7998897 |
| Beta-actin | 0.8457795 | 0.8832542 | 0.7946342 | 0.9267778 | 0.6435825 | 1.1258664 | 0.6400922 |
| Bax (alpha) | 1.0461246 | 1.0295255 | 1.037648 | 1.0487169 | 1.4329258 | 1.0629381 | 1.1681137 |
| Alpha 1 - inhibitor III | 0.6426812 | 0.6523874 | 0.7239008 | 0.7438422 | 0.6527166 | 1.0028344 | 0.6043494 |
| 60S ribosomal protein L6 | 1.0449053 | 1.0768906 | 0.809804 | 0.9494649 | 1.0966653 | 1.172357 | 1.3922592 |
| Phase-1 RCT-117 | 1.260836 | 1.488873 | 1.4134079 | 1.0557526 | 0.9472243 | 1.0675833 | 0.9922431 |
| Phase-1 RCT-102 | 1.0242358 | 0.7650408 | 0.7811276 | 0.9309548 | 0.5531166 | 0.5857912 | 0.3589506 |
| 3-hydroxyisobutyrate dehydrogenase | 0.8563317 | 1.263498 | 1.1132686 | 1.2752879 | 0.7166379 | 1.1311368 | 0.9569627 |
| ATPase inhibitor (rat mitochondrial IF1 | 0.9086809 | 1.523139 | 1.1182885 | 1.0891768 | 0.4391682 | 0.8301331 | 0.5848376 |
| protein) | | | | | | | |
| Alpha-2-macroglobulin | 0.7449276 | 1.6542026 | 0.7806153 | 1.4200066 | 0.5058997 | 0.7663649 | 0.4627633 |
| Phase-1 RCT-137 | 0.9648276 | 1.5168723 | 1.1995206 | 1.2559632 | 0.6695195 | 1.0140086 | 0.6744351 |
| Phase-1 RCT 252 | 0.6762629 | 0.831278 | 0.8637714 | 0.7857379 | 1.0141293 | 0.9032807 | 0.8484889 |
| Phase-1 RCT-65 | 1.0233536 | 0.9172255 | 1.0764174 | 1.0359195 | 2.0653486 | 1.6928691 | 1.6255807 |
| Liver fatty acid binding protein | 1.0148991 | 1.3544377 | 1.1863532 | 0.9321395 | 0.4524413 | 0.6080664 | 0.6717466 |
| Carbamyl phosphate synthetase I | 0.6687909 | 0.8536069 | 0.8474841 | 0.8190946 | 0.9329855 | 0.8940592 | 0.9712577 |
| c-jun | 1.0498438 | 0.9667493 | 1.0887262 | 1.0111239 | 1.3967819 | 0.9711213 | 0.8528442 |
| Transthyretin | 0.7892534 | 0.9569866 | 0.9584387 | 0.7100759 | 0.5367836 | 0.5899975 | 0.5991671 |
| Aflatoxin B1 aldehyde reductase | 0.7903845 | 0.9197916 | 0.7791598 | 0.7847079 | 0.8105843 | 1.2845099 | 1.1434147 |
| Phase-1 RCT-15 | 0.947639 | 1.2171261 | 1.0184524 | 0.9488336 | 1.7260218 | 1.5003911 | 1.8018925 |
| Insulin-like growth factor I | 0.7302675 | 1.1421229 | 1.0908562 | 1.1001841 | 0.5041788 | 0.6286917 | 0.6750658 |
| Sodium/bile acid cotransporter | 0.9131659 | 1.0396426 | 1.0869092 | 0.8449752 | 0.7477124 | 0.9879446 | 1.0380545 |
| Organic cation transporter 3 | 1.0968392 | 1.0472007 | 0.8988957 | 0.986372 | 0.9951441 | 0.9944876 | 1.1709911 |
| Gadd153 | 1.1202532 | 1.2809528 | 1.1410224 | 1.1584694 | 1.055819 | 0.9069458 | 1.0802016 |
| Phase-1 RCT-109 | 1.094703 | 1.2738577 | 1.0572506 | 1.0823812 | 1.1602619 | 1.0052907 | 1.195961 |
| Phase-1 RCT-88 | 0.8834708 | 1.0040814 | 1.336429 | 0.9864395 | 0.8336058 | 0.9802125 | 0.9469894 |
| Ubiquitin conjugating enzyme (RAD 6 | 1.0671748 | 0.8749884 | 0.7187884 | 0.9905873 | 0.916195 | 0.9249301 | 0.9684137 |
| homologue) | | | | | | | |
| Glucose-regulated protein 78 | 0.9597177 | 2.0454447 | 1.2520401 | 1.2555087 | 0.5912622 | 0.5333984 | 0.8723528 |
| Ribosomal protein L13A | 1.0554941 | 1.4414706 | 1.2252439 | 1.1640664 | 1.09293 | 1.1067629 | 1.1281542 |
| Monocyte chemotactic protein receptor | 1.1679579 | 0.9881861 | 0.9339516 | 0.936749 | 0.9340976 | 0.9509153 | 0.953972 |
| (CCR2) | | | | | | | |
| Fas antigen | 1.1997621 | 1.1113182 | 1.0798763 | 1.238301 | 0.6609684 | 0.8061969 | 0.7861994 |
| Calpactin I heavy chain | 1.0650861 | 1.3486049 | 1.2038094 | 1.1418315 | 1.5102588 | 1.2390724 | 1.3440775 |
| Uncoupling protein 2 | 1.2507656 | 1.073871 | 1.144688 | 1.0422362 | 1.0126103 | 1.0237933 | 0.819295 |
| Phase-1 RCT-168 | 1.0053369 | 1.0414331 | 0.9554883 | 1.0007696 | 1.0402021 | 0.7692204 | 0.8769292 |
| Phase-1 RCT-154 | 1.0284704 | 1.2491182 | 1.0622903 | 1.0341724 | 1.1362087 | 1.1200827 | 1.1436362 |
| Superoxide dismutase Mn | 1.2299312 | 3.5397284 | 1.620721 | 2.0311725 | 1.1768074 | 1.2895966 | 1.3674692 |
| Phase-1 RCT-214 | 1.0103006 | 1.1333699 | 1.1462982 | 1.0020114 | 0.829956 | 0.7476181 | 0.6585489 |
| Phase-1 RCT-225 | 0.9475496 | 0.8552404 | 0.6522903 | 1.1290088 | 1.5169756 | 1.8928087 | 0.6091376 |
| Phase-1 RCT-181 | 1.0266337 | 0.9184188 | 1.0248286 | 1.03 | 1.1418198 | 1.3074466 | 0.9403492 |
| Nucleoside diphosphate kinase beta | 1.1748644 | 1.5727006 | 1.4162894 | 1.4557424 | 1.0615329 | 1.3283691 | 1.4231929 |
| isoform | | | | | | | |
| Extracellular-signal-regulated kinase 1 | 0.9546834 | 1.3452194 | 0.8869863 | 1.0330003 | 1.1509323 | 1.3652226 | 1.2381482 |
| {Ribosomal protein L6} | 0.8910129 | 1.2770609 | 0.9694046 | 1.0013335 | 1.4658207 | 1.3217133 | 1.9163811 |
| Aldehyde dehydrogenase, microsomal | 1.0606048 | 1.2100272 | 1.0429099 | 1.0023754 | 0.9299008 | 1.1590096 | 0.7610748 |
| p53 | 1.1502441 | 1.2027552 | 0.9196404 | 1.038293 | 0.7966134 | 0.8437588 | 0.8255295 |
| Phase-1 RCT-239 | 0.8892142 | 0.8250758 | 0.9007312 | 0.8759945 | 1.47695 | 1.247851 | 1.1122367 |
| Macrophage inflammatory protein-2 alpha | 0.96162 | 1.2063959 | 1.2135922 | 1.0830756 | 1.0711341 | 1.0484394 | 1.1240859 |
| IgE binding protein | 0.9537656 | 1.1162554 | 1.1351768 | 1.0017319 | 1.3038343 | 1.0860491 | 1.1784645 |
| Phase-1 RCT-205 | 1.148797 | 0.9521009 | 0.9280353 | 1.0149823 | 0.9934965 | 1.0278277 | 1.0908263 |
| Melanoma-associated antigen ME491 | 1.1832527 | 1.1749369 | 1.0672234 | 1.111746 | 1.0148723 | 1.0271976 | 0.8852153 |
| Phase-1 RCT-242 | 1.2740754 | 0.8639044 | 0.8285046 | 0.9210899 | 1.1739111 | 0.8922104 | 0.9261866 |
| Enolase alpha | 1.1172845 | 1.7132647 | 1.5081278 | 1.5581639 | 1.0209273 | 1.0935841 | 1.7353852 |
| Thymosin beta-10 | 1.2781966 | 1.1220789 | 1.1501005 | 1.0699301 | 0.8798466 | 1.1026509 | 0.907828 |
| Cofilin | 0.9147836 | 1.1039462 | 1.0121367 | 0.8960105 | 1.3628459 | 1.4227997 | 1.2666644 |
| Carbonyl reductase | 1.1666231 | 0.8861219 | 0.9312707 | 1.0360234 | 0.8265593 | 0.7411223 | 0.6681411 |
| MAP kinase kinase | 0.8666886 | 1.0469087 | 1 | 1.0886178 | 0.8417131 | 0.8823712 | 1.119699 |
| Phase-1 RCT-162 | 1.0408144 | 1.4499726 | 1.0450523 | 1.0314128 | 1.216004 | 1.2265562 | 1.5253831 |
| Phase-1 RCT-72 | 0.9808678 | 0.9268482 | 0.9786701 | 0.8739393 | 1.3640453 | 1.0002636 | 1.4097186 |
| Ribosomal protein S8 | 0.9832621 | 1.8340534 | 1.2433833 | 1.2149522 | 1.2169771 | 1.4053944 | 2.206138 |
| Proliferating cell nuclear antigen gene | 1.2117277 | 1.0191394 | 1.0118906 | 0.961718 | 0.9794385 | 0.8792179 | 0.9541432 |
| Phase-1 RCT-192 | 1.2398323 | 1.4443008 | 1.2017136 | 1.0775869 | 1.4120709 | 1.6240517 | 1.5705532 |
| IkB-a | 0.967266 | 1.0511838 | 0.8995905 | 1.0205237 | 1.2906218 | 1.1539489 | 1.2569281 |
| Endogenous retroviral sequence, 5′ and | 0.8822105 | 1.0136741 | 0.589122 | 1.0823427 | 0.7625257 | 0.9590924 | 0.6334852 |
| 3′ LTR | | | | | | | |
| Phase-1 RCT-37 | 0.9908742 | 1.1896893 | 0.9577071 | 1.0089909 | 1.3397677 | 1.2501497 | 1.5043254 |
| Matrix metalloproteinase-1 | 0.9096614 | 1.1114557 | 1.2666249 | 1.1397347 | 0.8388416 | 0.9485998 | 1.032123 |
| Cyclin G | 1.0538665 | 1.1650543 | 1.0677277 | 1.0814815 | 1.6631067 | 1.3296047 | 1.8672763 |
| DNA polymerase beta | 0.9647466 | 1.2619385 | 1.159897 | 1.0941453 | 1.0308098 | 1.073167 | 1.099975 |
| Phase-1 RCT-127 | 1.2380753 | 1.1713632 | 1.1332923 | 1.0051208 | 1.0135945 | 0.9849969 | 1.0537654 |
| Multidrug resistant protein-2 | 1.0052876 | 0.9425771 | 1.0912297 | 1.006105 | 0.7789322 | 1.038398 | 1.0772347 |
| Ribosomal protein S9 | 1.1448232 | 1.3463068 | 1.1127572 | 1.2813442 | 0.8800685 | 1.0044005 | 0.9775681 |
| Phase-1 RCT-12 | 1.0972954 | 1.1846565 | 0.8915474 | 0.9915949 | 1.1153711 | 1.0728441 | 0.8380462 |
| Beta-tubulin, class I | 1.0664338 | 1.3792162 | 0.9915622 | 1.0126032 | 0.8973706 | 0.9248406 | 0.8424161 |
| Argininosuccinate lyase | 0.8723022 | 1.050779 | 1.0506321 | 0.8694069 | 2.8383193 | 1.4502248 | 1.9327924 |
| Elongation factor-1 alpha | 0.7992303 | 1.0948519 | 0.9959608 | 0.9681041 | 0.7833365 | 1.029647 | 1.0621622 |
| Phase-1 RCT-55 | 0.8072829 | 0.8908684 | 0.8394074 | 0.8284411 | 0.9751847 | 0.8015461 | 0.989175 |
| Macrophage inflammatory protein-1 alpha | 1.3505888 | 0.9156402 | 0.8723202 | 1.0605185 | 0.8927752 | 0.9258928 | 0.8537576 |
| c-H-ras | 0.9218839 | 1.1451765 | 1.0243174 | 1.0140023 | 1.2371001 | 1.1112157 | 1.2343993 |
| Multidrug resistant protein-1 | 0.9503454 | 1.1687711 | 1.1595697 | 1.0658762 | 0.7772039 | 0.9947537 | 0.9486536 |
| T-cell cyclophilin | 0.9397931 | 1.338579 | 1.1714432 | 1.1766771 | 0.8282061 | 1.0330414 | 0.9489519 |
| Phase-1 RCT-185 | 0.7263861 | 0.733953 | 0.883491 | 0.9602943 | 0.8169107 | 0.9969682 | 0.907629 |
| 25-DX | 0.9140533 | 0.9927501 | 0.894019 | 0.9292117 | 0.5866038 | 0.965022 | 0.5424061 |
| Ref-1 | 0.9185734 | 1.1970148 | 1.2041531 | 0.9080426 | 1.2696769 | 0.9478902 | 1.501708 |
| Apolipoprotein CIII | 1.0957452 | 0.9154562 | 0.90213 | 0.8632187 | 0.6120905 | 0.7641302 | 0.517848 |
| NADPH quinone oxidoreductase-1 (DT- | 0.9042904 | 1.364032 | 1.3053478 | 0.9907369 | 1.4194748 | 1.3612328 | 1.5495567 |
| diaphorase) | | | | | | | |
| Pyruvate kinase, muscle | 0.9989268 | 1.0069858 | 1.0461496 | 1.0201097 | 1.3594375 | 1.0729209 | 1.2579149 |
| | AZA | AZA | AZA | BEN | BEN | BEN | BEN |
| Compound-Dose (2) | 200 | 200 | 200 | 250 | 250 | 250 | 1000 |
| Animal Number (3) | 1834 | 1835 | 1836 | 2024 | 2025 | 2026 | 2034 |
| Liver Toxicity Necrosis Classification (4) | no | no | no | no | no | no | no |
| Gene Name (5) | | | | | | | |
| Phase-1 RCT-78 | 0.6966337 | 0.76912 | 0.5239551 | 0.9292451 | 1.005316 | 0.9592632 | 1.1148616 |
| Gamma-actin, cytoplasmic | 3.7113097 | 2.4193583 | 2.9596164 | 1.1211765 | 1.0631752 | 0.8050485 | 1.3450783 |
| Matrin F/G | 1.597515 | 1.1693022 | 1.4484042 | 1.2323203 | 1.1039815 | 1.0824697 | 1.3069142 |
| Zinc finger protein | 3.7833471 | 2.9543152 | 1.678247 | 1.0909039 | 1.0063345 | 1.0674199 | 0.9449763 |
| Cathepsin L, sequence 2 | 4.1413903 | 4.82356 | 4.0171266 | 1.0810305 | 1.0625975 | 1.4416926 | 0.8298467 |
| Gadd45 | 1.3841097 | 1.3486642 | 0.9782585 | 1.1186035 | 0.8869724 | 0.9174147 | 1.0454173 |
| Phase-1 RCT-144 | 1.5608697 | 1.1077926 | 1.7616754 | 0.9282573 | 1.076135 | 0.9790328 | 0.97044 |
| Phase-1 RCT-145 | 2.322422 | 1.7900894 | 2.3341157 | 0.9507642 | 1.0198053 | 0.996983 | 0.9651043 |
| Phase-1 RCT-50 | 2.0424013 | 1.8918456 | 1.4971056 | 0.9201075 | 0.9508708 | 1.1421466 | 0.9221596 |
| Phase-1 RCT-92 | 0.3981309 | 0.3829268 | 0.5293839 | 1.1732237 | 1.1429317 | 0.8775197 | 1.0870357 |
| Phase-1 RCT-89 | 0.5954438 | 0.5911951 | 0.6828815 | 1.0488504 | 1.0163027 | 0.9257058 | 1.1293125 |
| 14-3-3 zeta | 1.8646452 | 1.6531961 | 1.6096697 | 0.9208501 | 0.8478094 | 0.881484 | 0.9486937 |
| Dynamin-1 (D100) | 0.8447272 | 0.7388033 | 0.7987577 | 1.0105406 | 1.0823545 | 0.9226728 | 1.0720388 |
| Insulin-like growth factor binding protein 1 | 7.4772077 | 9.646749 | 1.6741246 | 1.2367438 | 1.2538363 | 1.2067088 | 1.217728 |
| L-gulono-gamma-lactone oxidase | 0.3925192 | 0.3399565 | 0.289002 | 1.2729362 | 1.1302453 | 0.9740832 | 1.2142392 |
| Ornithine decarboxylase | 2.6674852 | 2.3179746 | 3.1643207 | 1.0829935 | 0.7960098 | 1.0381306 | 1.2280579 |
| PAR interacting protein | 2.7082071 | 1.7582772 | 2.5890565 | 0.9145443 | 1.1607314 | 0.9615644 | 1.0912352 |
| Phase-1 RCT-128 | 0.7246171 | 0.4810762 | 0.4338843 | 1.0329999 | 1.1524246 | 0.9332992 | 1.1424961 |
| Phase-1 RCT-180 | 1.5572282 | 1.3217336 | 1.2062231 | 1.0153166 | 1.1699657 | 0.8752314 | 1.0433127 |
| Phase-1 RCT-182 | 0.9087691 | 1.0611457 | 0.661459 | 0.9595929 | 1.1186393 | 1.0371811 | 0.9949247 |
| Phase-1 RCT-207 | 3.581343 | 2.201169 | 2.083282 | 0.9522653 | 1.0296407 | 1.0891997 | 1.0198023 |
| Phase-1 RCT-213 | 1.1855916 | 1.1017321 | 1.0299644 | 0.9845672 | 1.0451869 | 1.0561191 | 1.0463935 |
| Phase-1 RCT-256 | 0.6239309 | 0.5814197 | 0.5354769 | 1.2432363 | 1.3048334 | 0.8207673 | 1.1988181 |
| Phase-1 RCT-258 | 1.5766476 | 1.3367516 | 1.382023 | 0.9957253 | 1.1072593 | 1.0775684 | 0.9812813 |
| Phase-1 RCT-264 | 0.5792218 | 0.6164239 | 0.4954063 | 1.1532516 | 1.1784669 | 1.1830523 | 1.1864895 |
| Phase-1 RCT-271 | 0.8163146 | 0.908581 | 0.7150847 | 1.0753878 | 1.1988932 | 0.9673495 | 1.167427 |
| Phase-1 RCT-288 | 0.5175948 | 0.6436232 | 0.4976981 | 1.2267514 | 1.0697432 | 1.2011045 | 0.8798338 |
| Phase-1 RCT-38 | 0.5638173 | 0.6010841 | 0.4800534 | 1.2624466 | 1.3171362 | 0.8318521 | 1.0996178 |
| Phase-1 RCT-39 | 1.3282126 | 1.2363253 | 1.2692422 | 0.9494178 | 0.8630604 | 1.0180182 | 0.9362262 |
| Phase-1 RCT-68 | 0.977351 | 1.668641 | 1.2330366 | 1.0292512 | 0.9737785 | 1.060279 | 1.0595927 |
| Phase-1 RCT-33 | 0.5462915 | 0.6388092 | 0.5240665 | 1.3416235 | 1.0231903 | 0.8933977 | 1.0218835 |
| Phase-1 RCT-36 | 0.8427327 | 0.7448815 | 0.9063669 | 1.2326769 | 0.9993069 | 0.9156223 | 0.981406 |
| 17-beta hydroxysteroid dehydrogenase, | 1.0372355 | 0.387267 | 0.9144725 | 1.4559753 | 0.7260974 | 2.0725243 | 0.7284895 |
| type 2 | | | | | | | |
| Senescence marker protein-30 | 0.1043787 | 0.0929194 | 0.1143656 | 1.1751034 | 1.1928709 | 1.1474496 | 1.0320355 |
| Ribosomal protein S17 | 2.5854347 | 2.4897707 | 2.0641575 | 1.0818571 | 1.5008307 | 1.1447939 | 1.3563975 |
| Phase-1 RCT-83 | 1.0906727 | 0.8314614 | 1.0777624 | 0.8660462 | 0.9194189 | 0.7271524 | 1.0765259 |
| Phase-1 RCT-49 | 1.4146855 | 0.7749877 | 1 | 0.9541073 | 1.0696465 | 0.8911991 | 0.8915008 |
| Phase-1 RCT-48 | 0.8615946 | 0.7880989 | 0.8058059 | 0.7851294 | 0.753834 | 0.7570276 | 1.1410823 |
| Phase-1 RCT-40 | 1.0034037 | 0.9530306 | 0.8039673 | 0.9584477 | 1.0564356 | 1.0212655 | 1.2086035 |
| Phase-1 RCT-296 | 0.3682909 | 0.1969152 | 0.2741845 | 1.2441527 | 0.909405 | 0.6119775 | 0.7078477 |
| Phase-1 RCT-295 | 1.8207892 | 2.4026294 | 1.5569768 | 1.072399 | 1.007202 | 1.0133826 | 1.290285 |
| Phase-1 RCT-291 | 0.8321025 | 0.8445127 | 0.6477352 | 1.0311136 | 1.0822281 | 1.0381668 | 0.9800899 |
| Phase-1 RCT-270 | 0.3171353 | 0.3314283 | 0.2660066 | 1.0055592 | 1.0221418 | 1.0584432 | 1.230621 |
| Phase-1 RCT-241 | 2.8140347 | 2.53466 | 2.9338982 | 0.9618745 | 0.9668073 | 0.8652557 | 0.937066 |
| Phase-1 RCT-191 | 2.3215384 | 1.7151371 | 2.396179 | 1.0358827 | 0.9574447 | 0.9776347 | 1.4443167 |
| Phase-1 RCT-189 | 0.5559768 | 0.4622004 | 0.4814428 | 1.0086552 | 1.1220528 | 1.1701125 | 1.1136184 |
| Phase-1 RCT-179 | 3.400019 | 2.5933642 | 2.2101238 | 1.0832627 | 1.0011777 | 1.0370518 | 0.8289163 |
| Phase-1 RCT-152 | 1.9038615 | 2.3436167 | 2.026818 | 0.934406 | 1.1481379 | 1.0084034 | 1.0914087 |
| Phase-1 RCT-123 | 0.8852586 | 0.9679442 | 0.9143729 | 1.0375941 | 0.9776238 | 0.9152062 | 0.9115186 |
| Paraoxonase 1 | 0.5513634 | 0.5483122 | 0.3947786 | 1.0723425 | 1.1213222 | 1.1918526 | 0.8321396 |
| Organic anion transporter 3 | 0.4497929 | 0.6878383 | 0.8383967 | 1.1012778 | 0.9735543 | 1.023828 | 0.9536997 |
| N-hydroxy-2-acetylaminofluorene | 0.2549956 | 0.3152328 | 0.3613176 | 0.9549221 | 0.9815674 | 0.912648 | 1.0523893 |
| sulfotransferase (ST1C1) | | | | | | | |
| Integrin beta1 | 1.2875762 | 1.4129983 | 1.1690544 | 1.0813729 | 1.2109368 | 1.1266248 | 1.0635471 |
| Insulin-like growth factor binding protein 3 | 0.6730096 | 0.8269937 | 0.5633904 | 0.8956429 | 1.0516661 | 0.9340481 | 0.879999 |
| ID-1 | 1.6034024 | 1.0984336 | 1.5095127 | 0.9398435 | 0.9273287 | 0.9904381 | 0.8907645 |
| Hepatic lipase | 0.4828872 | 0.4433413 | 0.3662966 | 1.1180252 | 1.3373758 | 0.7376425 | 0.8900666 |
| Heme oxygenase | 2.2231264 | 1.9260212 | 1.1356058 | 0.9820411 | 0.9306974 | 1.2183154 | 0.8456153 |
| Equilbrative nitrobenzylthioinosine- | 0.4699491 | 0.495319 | 0.5670274 | 0.9641964 | 0.9726889 | 0.874571 | 1.0759777 |
| sensitive nucleoside transporter | | | | | | | |
| Epidermal growth factor | 0.8700575 | 1.1877998 | 0.9476621 | 0.9158884 | 0.9254838 | 0.9170199 | 1.0217446 |
| c-myc | 2.955166 | 2.4390032 | 1.5227859 | 1.087673 | 0.9170278 | 0.8689754 | 1.0511134 |
| Carbonic anhydrase III | 0.1128292 | 0.2098177 | 0.0979962 | 0.9267532 | 0.804558 | 0.6483697 | 1.9800402 |
| Beta-actin | 2.1455996 | 1.209875 | 1.783 | 1.0198948 | 0.5883343 | 0.7476708 | 1.155623 |
| Bax (alpha) | 2.1796682 | 1.5512561 | 1.7595508 | 0.9815292 | 1.0086665 | 0.8919374 | 0.9499462 |
| Alpha 1 - inhibitor III | 0.535313 | 0.4228615 | 0.3517941 | 1.1266437 | 1.377275 | 0.7856618 | 1.1728764 |
| 60S ribosomal protein L6 | 2.109469 | 2.2923336 | 1.7147254 | 0.9693109 | 1.2186768 | 1.0584786 | 1.0068545 |
| Phase-1 RCT-117 | 0.7306401 | 0.9177682 | 0.7440881 | 0.8857632 | 0.9398662 | 0.794009 | 0.7255443 |
| Phase-1 RCT-102 | 0.4125425 | 0.2763322 | 0.3086535 | 0.9749973 | 1.0985925 | 0.508824 | 0.7869762 |
| 3-hydroxyisobutyrate dehydrogenase | 0.7314415 | 0.7112612 | 0.6382471 | 1.1120981 | 1.0662285 | 1.1180098 | 1.2129534 |
| ATPase inhibitor (rat mitochondrial IF1 | 0.3957389 | 0.4425302 | 0.3983508 | 1.0099877 | 1.1223562 | 1.1052583 | 0.982656 |
| protein) | | | | | | | |
| Alpha-2-macroglobulin | 0.3604016 | 0.6775749 | 0.4719652 | 1.1477631 | 1.4129804 | 1.0401325 | 0.9729531 |
| Phase-1 RCT-137 | 0.6888283 | 0.8855293 | 0.6477727 | 1.1329114 | 1.2717985 | 1.0440713 | 0.9050218 |
| Phase-1 RCT 252 | 0.8883688 | 1.0348597 | 0.764273 | 1.0626657 | 1.1786468 | 1.0468917 | 1.0463514 |
| Phase-1 RCT-65 | 1.4580855 | 1.8278515 | 1.7081406 | 1.021939 | 0.9481224 | 0.9519528 | 1.2516361 |
| Liver fatty acid binding protein | 0.3070056 | 0.3965497 | 0.2404319 | 1.0808522 | 1.4414902 | 0.9398744 | 1.0131907 |
| Carbamyl phosphate synthetase I | 0.878952 | 1.2114036 | 0.572312 | 1.0945495 | 1.232482 | 1.1601917 | 1.0695795 |
| c-jun | 1.1384608 | 1.2323586 | 1.3022552 | 0.9204963 | 0.7828084 | 1.1115937 | 1.2109932 |
| Transthyretin | 0.4767853 | 0.3951052 | 0.2835472 | 0.925694 | 1.0649593 | 1.0430142 | 1.0870161 |
| Aflatoxin B1 aldehyde reductase | 2.2256725 | 1.3961154 | 1.3114012 | 1.3006419 | 1.3652612 | 1.1129129 | 1.6912673 |
| Phase-1 RCT-15 | 1.10744 | 1.9384079 | 1.5923443 | 0.9557661 | 1.0504875 | 1.1361768 | 1.3270931 |
| Insulin-like growth factor I | 0.7006372 | 0.7520742 | 0.4446287 | 0.9035857 | 1.0632257 | 0.8187211 | 0.8566466 |
| Sodium/bile acid cotransporter | 0.3544989 | 0.5923152 | 0.420512 | 1.153714 | 1.1635811 | 0.9872644 | 1.0882746 |
| Organic cation transporter 3 | 1.2896361 | 1.355899 | 1.4426008 | 1.001565 | 1.1819994 | 1.0736699 | 0.9742056 |
| Gadd153 | 1.5431488 | 1.6952058 | 1.5897092 | 0.906382 | 0.9664307 | 0.8976809 | 0.9358965 |
| Phase-1 RCT-109 | 2.0017002 | 1.5736568 | 1.9256737 | 1.013805 | 1.2096984 | 1.0263801 | 1.100054 |
| Phase-1 RCT-88 | 0.8937986 | 0.9339541 | 0.898189 | 1.0867556 | 1.0979505 | 0.896956 | 1.0120294 |
| Ubiquitin conjugating enzyme (RAD 6 | 1.4982063 | 1.549052 | 1.1318076 | 0.9573855 | 1.0963624 | 1.0298058 | 1.011227 |
| homologue) | | | | | | | |
| Glucose-regulated protein 78 | 1.26832 | 1.532798 | 2.1707742 | 0.8874235 | 1.1607352 | 1.1412001 | 0.883291 |
| Ribosomal protein L13A | 2.3670874 | 2.1578383 | 1.5810844 | 0.9783386 | 1.1213123 | 1.0636284 | 1.049043 |
| Monocyte chemotactic protein receptor | 1.2276454 | 1.1490645 | 1.3174213 | 0.994109 | 1.0987456 | 1.0130931 | 0.9461094 |
| (CCR2) | | | | | | | |
| Fas antigen | 1.3866451 | 1.2914145 | 1.3099786 | 0.981465 | 0.7583471 | 0.8983236 | 1.0946722 |
| Calpactin I heavy chain | 2.292488 | 1.0395428 | 1.3612175 | 0.93792 | 0.9210932 | 0.9379831 | 1.1759462 |
| Uncoupling protein 2 | 1.3006057 | 0.9405885 | 0.9984959 | 0.8809454 | 0.9842479 | 0.9005647 | 0.9186463 |
| Phase-1 RCT-168 | 0.8597759 | 0.7851655 | 0.7220254 | 1.0482829 | 1.1402363 | 1.1048865 | 1.0036719 |
| Phase-1 RCT-154 | 3.2134542 | 2.3379166 | 2.8159227 | 0.9737443 | 1.0869392 | 0.9941529 | 0.9363217 |
| Superoxide dismutase Mn | 2.2236707 | 2.6636457 | 1.320855 | 1.0570674 | 0.968507 | 1.0107254 | 1.2044377 |
| Phase-1 RCT-214 | 0.4200544 | 0.6125698 | 0.40149 | 1.2329315 | 0.9380732 | 1.0429778 | 0.8738685 |
| Phase-1 RCT-225 | 1.4242824 | 0.8452123 | 1.1055099 | 1.2475954 | 0.8390377 | 0.7503263 | 0.9781839 |
| Phase-1 RCT-181 | 1.0853324 | 0.8594197 | 0.7264623 | 1.0027984 | 0.9606602 | 0.8368456 | 0.8557891 |
| Nucleoside diphosphate kinase beta | 1.6000621 | 2.075579 | 1.4382755 | 1.0324707 | 1.1152691 | 0.9311169 | 1.1390778 |
| isoform | | | | | | | |
| Extracellular-signal-regulated kinase 1 | 0.8093335 | 0.7986614 | 0.5854928 | 0.896482 | 0.8874506 | 0.7024323 | 1.8199605 |
| {Ribosomal protein L6} | 2.437476 | 2.31947 | 2.1404097 | 1.0303496 | 1.1675267 | 1.0800574 | 1.1385733 |
| Aldehyde dehydrogenase, microsomal | 0.8902168 | 1.0531714 | 0.6068111 | 1.2564394 | 1.0522046 | 1.0734015 | 1.0419233 |
| p53 | 1.6350684 | 1.549507 | 1.4160074 | 0.9617775 | 1.1536652 | 0.9543483 | 0.9370994 |
| Phase-1 RCT-239 | 0.6479248 | 0.8278147 | 1.1579431 | 0.9482529 | 1.0110567 | 0.9096378 | 1.3171164 |
| Macrophage inflammatory protein-2 alpha | 2.8009555 | 1.5657178 | 1.4802667 | 0.9296439 | 1.0333728 | 0.9984052 | 0.9315205 |
| IgE binding protein | 2.0368078 | 1.1205053 | 1.2887799 | 0.9922071 | 0.9552927 | 1.0428023 | 0.9253113 |
| Phase-1 RCT-205 | 1.4595677 | 1.0801259 | 1.2561373 | 0.9881126 | 1.1834087 | 0.9485072 | 0.9858624 |
| Melanoma-associated antigen ME491 | 1.183064 | 1.1525198 | 1.0177659 | 0.8850345 | 1.1398335 | 1.1886117 | 1.1011051 |
| Phase-1 RCT-242 | 2.7421343 | 2.072074 | 1.7595274 | 0.9358765 | 1.0192795 | 0.8450537 | 0.9359088 |
| Enolase alpha | 2.007912 | 2.179798 | 2.0758271 | 1.0282851 | 1.2636331 | 0.9454425 | 1.1175112 |
| Thymosin beta-10 | 1.5735146 | 1.3253361 | 0.9694758 | 0.9513483 | 1.0328171 | 0.9745186 | 0.9128686 |
| Cofilin | 2.004856 | 1.9089888 | 1.7015514 | 1.0773121 | 1.1267308 | 1.0939043 | 1.1557666 |
| Carbonyl reductase | 1.3698994 | 1.4838775 | 1.3819762 | 0.9991375 | 0.9812979 | 0.9882416 | 0.9825527 |
| MAP kinase kinase | 0.9775976 | 1.060353 | 0.7803983 | 0.962752 | 0.8489384 | 1.0266925 | 1.0075188 |
| Phase-1 RCT-162 | 1.4439982 | 1.3182963 | 1.4743998 | 1.0035086 | 1.1285129 | 1.0472312 | 1.1286196 |
| Phase-1 RCT-72 | 0.5506171 | 1.1688455 | 1.1948105 | 0.9500626 | 0.9733757 | 0.9486521 | 1.0914369 |
| Ribosomal protein S8 | 2.5998082 | 2.4200976 | 2.2287722 | 1.0324107 | 1.4452399 | 1.1289481 | 1.2236403 |
| Proliferating cell nuclear antigen gene | 1.161188 | 1.2172091 | 1.0723077 | 0.9951933 | 0.9013112 | 0.8948703 | 0.8367055 |
| Phase-1 RCT-192 | 2.3663833 | 2.7402613 | 2.2067914 | 1.0858628 | 1.0200282 | 1.0567776 | 1.0074214 |
| IkB-a | 1.9840299 | 1.5882152 | 1.4755849 | 0.9986046 | 1.2946097 | 0.9912198 | 1.2760468 |
| Endogenous retroviral sequence, 5′ and | 0.9938678 | 0.6176182 | 0.689525 | 1.232518 | 0.4768834 | 1.1455748 | 1.022733 |
| 3′ LTR | | | | | | | |
| Phase-1 RCT-37 | 1.1384115 | 1.2056628 | 1.2829539 | 1.0380094 | 1.1082013 | 1.0264146 | 1.0973363 |
| Matrix metalloproteinase-1 | 1.3651036 | 1.0617216 | 1.1747607 | 1.0981578 | 1.302976 | 1.2203318 | 1.1522845 |
| Cyclin G | 4.1628447 | 5.219233 | 4.203121 | 1.0759602 | 1.0838652 | 0.8941499 | 1.0132829 |
| DNA polymerase beta | 1.3806723 | 1.5133308 | 1.3679365 | 1.1280129 | 1.2439889 | 1.1131299 | 1.102701 |
| Phase-1 RCT-127 | 1.760293 | 1.8099358 | 1.359161 | 0.9493663 | 0.9927587 | 0.8875207 | 0.822957 |
| Multidrug resistant protein-2 | 2.8270404 | 1.8727441 | 2.0099854 | 0.9262759 | 0.7926945 | 1.0984764 | 0.9789383 |
| Ribosomal protein S9 | 1.8721043 | 1.6732374 | 1.1191888 | 0.8987832 | 1.0575777 | 1.0042931 | 1.0534278 |
| Phase-1 RCT-12 | 1.1861581 | 0.8096998 | 1.2324103 | 0.9713572 | 0.8793325 | 0.8743198 | 1.0794019 |
| Beta-tubulin, class I | 1.6286935 | 0.7008052 | 1.4540414 | 1.0390564 | 1.0174049 | 0.7305253 | 1.2538594 |
| Argininosuccinate lyase | 5.57864 | 4.1464057 | 2.867889 | 1.00485 | 0.8963179 | 1.2799237 | 1.176554 |
| Elongation factor-1 alpha | 2.0919669 | 2.057847 | 1.3752365 | 1.0890957 | 1.1157644 | 1.2466987 | 1.3338935 |
| Phase-1 RCT-55 | 0.5973496 | 1.327144 | 1.7170528 | 1.0275701 | 1.0070853 | 0.8074127 | 1.1874329 |
| Macrophage inflammatory protein-1 alpha | 1.3285068 | 0.8618391 | 1.1024398 | 1.0068718 | 1.1206398 | 0.9337961 | 0.9030241 |
| c-H-ras | 1.3059788 | 1.327043 | 1.2896405 | 1.0363495 | 0.9856399 | 1.1840907 | 1.0482775 |
| Multidrug resistant protein-1 | 3.4321015 | 2.0541956 | 2.3662581 | 0.9083537 | 0.9137271 | 1.0225953 | 1.0110735 |
| T-cell cyclophilin | 1.3834865 | 1.5060042 | 1.20479 | 1.1610852 | 1.2674953 | 1.0952082 | 1.1426393 |
| Phase-1 RCT-185 | 0.7562463 | 0.8287317 | 0.7280609 | 1.1136601 | 1.0350257 | 0.8967831 | 1.0487007 |
| 25-DX | 0.8739833 | 0.8522831 | 0.7223803 | 1.2626667 | 1.3260094 | 1.2735499 | 0.9572457 |
| Ref-1 | 0.7942405 | 1.5546198 | 1.6225113 | 1.0094053 | 1.0439547 | 1.030285 | 1.0586076 |
| Apolipoprotein CIII | 0.6241902 | 0.5841411 | 0.4123555 | 0.9432988 | 0.9851726 | 0.8685129 | 0.9614223 |
| NADPH quinone oxidoreductase-1 (DT- | 1.7295493 | 1.9655155 | 1.7247624 | 1.1085218 | 1.0395936 | 1.1150974 | 1.045258 |
| diaphorase) | | | | | | | |
| Pyruvate kinase, muscle | 1.1416324 | 0.7298476 | 0.8793267 | 1.0196491 | 1.0080576 | 1.0370488 | 0.9573476 |
| | BEN | BEN | BAP | BAP | BAP | BUS | BUS |
| Compound-Dose (2) | 1000 | 1000 | 30 | 30 | 30 | 14 | 14 |
| Animal Number (3) | 2035 | 2036 | 2344 | 2345 | 2346 | 1744 | 1745 |
| Liver Toxicity Necrosis Classification (4) | no | no | no | no | no | no | no |
| Gene Name (5) | | | | | | | |
| Phase-1 RCT-78 | 1.012998 | 1.0248717 | 0.9888582 | 0.8693752 | 0.8357989 | 1.064394 | 1.0704983 |
| Gamma-actin, cytoplasmic | 0.9904965 | 0.916226 | 0.9347313 | 1.012718 | 0.8351032 | 1.397714 | 1.49204 |
| Matrin F/G | 1.3284292 | 1.2325181 | 1.5234137 | 1.0414155 | 0.8653234 | 1.3978662 | 1.1594794 |
| Zinc finger protein | 0.9700281 | 1.0248989 | 1.2265573 | 1.045693 | 0.8733212 | 0.9783377 | 1.3610433 |
| Cathepsin L, sequence 2 | 1.0271238 | 0.9386216 | 2.8414452 | 1.4512879 | 1.6239613 | 1.0440164 | 1.1563909 |
| Gadd45 | 1.0248128 | 1.1098864 | 1.3675758 | 1.3047723 | 1.6485288 | 0.8359598 | 0.7524573 |
| Phase-1 RCT-144 | 0.9582144 | 0.9692081 | 0.9444003 | 0.9053437 | 1.007073 | 0.901138 | 0.9892833 |
| Phase-1 RCT-145 | 1.0088658 | 1.2072213 | 1.0712887 | 0.9765844 | 1.070357 | 0.9624114 | 0.9869101 |
| Phase-1 RCT-50 | 0.916575 | 0.9012362 | 0.9081509 | 0.9415907 | 0.9266058 | 0.7655572 | 0.7994906 |
| Phase-1 RCT-92 | 0.9555517 | 1.0321226 | 0.785619 | 0.8940618 | 1.0910436 | 1.074838 | 1.1755104 |
| Phase-1 RCT-89 | 0.9675681 | 0.8607842 | 0.9187344 | 0.9063843 | 1.0859551 | 1.2126415 | 1.120274 |
| 14-3-3 zeta | 1.0424389 | 0.9743778 | 0.9276852 | 1.0456921 | 0.9326912 | 1.3681663 | 1.1481848 |
| Dynamin-1 (D100) | 0.9048159 | 0.9281927 | 0.8574143 | 0.7524779 | 0.9661824 | 0.7779045 | 0.845993 |
| Insulin-like growth factor binding protein 1 | 1.2859656 | 1.0474768 | 2.1500325 | 1.1949488 | 1.141738 | 0.9980807 | 0.9117706 |
| L-gulono-gamma-lactone oxidase | 0.8097124 | 1.1911863 | 0.7264397 | 1.0787615 | 1.4016356 | 1.5709223 | 1.5704963 |
| Ornithine decarboxylase | 0.978526 | 0.9153351 | 1.0119632 | 1.0287522 | 1.1766845 | 1.1798028 | 1.4534361 |
| PAR interacting protein | 0.9760183 | 1.005397 | 1.0038546 | 0.9903606 | 1.0913972 | 0.9829292 | 1.0427082 |
| Phase-1 RCT-128 | 0.9317038 | 0.9421548 | 0.936469 | 0.7248523 | 1.0672041 | 1.4167204 | 1.2491512 |
| Phase-1 RCT-180 | 0.9300399 | 0.8989515 | 0.7909178 | 0.990127 | 1.1647229 | 1.5064911 | 1.7705806 |
| Phase-1 RCT-182 | 0.9571756 | 1.0689924 | 1.0125006 | 0.7540172 | 1.001911 | 1.1237267 | 1.0449928 |
| Phase-1 RCT-207 | 0.9402853 | 0.9895836 | 1.143769 | 0.9253087 | 0.9841292 | 1.086318 | 1.019465 |
| Phase-1 RCT-213 | 1.0430176 | 1.2194318 | 1.064771 | 1.0367198 | 0.9756231 | 1.1261985 | 1.0382159 |
| Phase-1 RCT-256 | 1.0822227 | 1.0938944 | 1.021899 | 1.0361824 | 1.4391258 | 1.3993646 | 1.2264366 |
| Phase-1 RCT-258 | 1.024189 | 1.1017636 | 1.0585428 | 0.9336639 | 0.996589 | 1.0208126 | 1.0018185 |
| Phase-1 RCT-264 | 0.6786357 | 1.1560445 | 0.8458368 | 0.6209406 | 0.9230838 | 1.2355839 | 1.4836521 |
| Phase-1 RCT-271 | 0.9232683 | 0.9112026 | 0.7968035 | 0.7628693 | 1.0253656 | 1.4306257 | 1.2564017 |
| Phase-1 RCT-288 | 0.9481354 | 0.9404666 | 1.1046783 | 0.7521556 | 0.8453296 | 1.4246571 | 1.5688425 |
| Phase-1 RCT-38 | 1.171769 | 1.0876458 | 1.1061925 | 1.1500988 | 1.5118519 | 1.3216703 | 1.1581867 |
| Phase-1 RCT-39 | 1.2119607 | 0.9433367 | 1.0758175 | 1.093325 | 0.973535 | 0.907436 | 1.1731839 |
| Phase-1 RCT-68 | 1.037136 | 1.1787753 | 1.2427366 | 1.0319047 | 1.117024 | 0.8833348 | 0.876555 |
| Phase-1 RCT-33 | 1.0051633 | 0.9833072 | 0.826028 | 0.8453579 | 0.9376368 | 1.2807688 | 1.3484479 |
| Phase-1 RCT-36 | 0.9860046 | 0.8883501 | 0.8844469 | 0.9325002 | 0.9857375 | 0.9250174 | 0.9103485 |
| 17-beta hydroxysteroid dehydrogenase, | 0.8330053 | 2.8086312 | 0.3757527 | 0.9452004 | 0.8252792 | 0.5919523 | 0.8079439 |
| type 2 | | | | | | | |
| Senescence marker protein-30 | 0.65751 | 0.8170337 | 1.0364783 | 1.4015683 | 1.809052 | 0.8709043 | 1.2688149 |
| Ribosomal protein S17 | 1.3885648 | 1.7355832 | 1.4411916 | 1.0686182 | 1.4209476 | 1.0922575 | 1.0512481 |
| Phase-1 RCT-83 | 1.1457834 | 0.9359222 | 1.1845433 | 1.0804242 | 0.9613739 | 0.9122688 | 0.7133207 |
| Phase-1 RCT-49 | 0.9685266 | 0.935126 | 0.9552386 | 0.9054073 | 0.9258575 | 0.8324286 | 0.8775227 |
| Phase-1 RCT-48 | 0.7717118 | 1.0991498 | 0.6532858 | 0.7383797 | 0.9898392 | 1.400528 | 1.2530143 |
| Phase-1 RCT-40 | 0.8928828 | 0.8817722 | 0.97268 | 0.946736 | 1.0502566 | 1.2375789 | 1.0527813 |
| Phase-1 RCT-296 | 0.8873732 | 0.9964241 | 0.3556601 | 0.6152238 | 1.2677956 | 1.0469518 | 1.5326462 |
| Phase-1 RCT-295 | 1.167717 | 1.5818933 | 1.1372108 | 1.0487927 | 1.2617238 | 1.3424479 | 1.4007715 |
| Phase-1 RCT-291 | 1.03217 | 0.9643396 | 1.1064429 | 0.9744304 | 1.237043 | 1.1395005 | 1.2189777 |
| Phase-1 RCT-270 | 0.917407 | 1.1043437 | 0.7610961 | 0.7352368 | 0.9465089 | 1.3650941 | 1.403988 |
| Phase-1 RCT-241 | 1.1699233 | 0.9929033 | 0.8207184 | 0.9238097 | 0.8705906 | 0.8699716 | 0.8804184 |
| Phase-1 RCT-191 | 1.2391211 | 1.3852113 | 0.9640195 | 1.2269036 | 1.0533156 | 1.1203219 | 1.1333405 |
| Phase-1 RCT-189 | 0.9118889 | 1.0496056 | 1.087906 | 0.8967375 | 0.8996317 | 1.2876908 | 1.1095145 |
| Phase-1 RCT-179 | 0.945356 | 0.9862577 | 1.2491074 | 0.9168077 | 1.1163371 | 1.472374 | 1.4217837 |
| Phase-1 RCT-152 | 1.0043366 | 1.215023 | 1.4748924 | 1.0913185 | 1.2602001 | 1.1685336 | 1.1325012 |
| Phase-1 RCT-123 | 0.9126924 | 0.8678719 | 0.9168926 | 0.9134544 | 0.9342126 | 0.7620599 | 0.8202166 |
| Paraoxonase 1 | 0.8268871 | 0.8769804 | 1.5926524 | 1.3103069 | 1.732061 | 1.0387398 | 1.1294318 |
| Organic anion transporter 3 | 1.0919858 | 0.9814556 | 0.7736386 | 0.9169813 | 0.939876 | 0.9774919 | 0.9133499 |
| N-hydroxy-2-acetylaminofluorene | 1.2117546 | 0.9203163 | 1.0873955 | 1.1490673 | 1.3631811 | 1.0586367 | 0.9530183 |
| sulfotransferase (ST1C1) | | | | | | | |
| Integrin beta1 | 1.0215065 | 1.0833187 | 1.6090201 | 1.0377716 | 0.8962543 | 1.1605645 | 1.0938702 |
| Insulin-like growth factor binding protein 3 | 0.9452874 | 1.1773409 | 0.8141185 | 0.7554984 | 0.7933477 | 1.1176977 | 1.1892314 |
| ID-1 | 1.107624 | 1.1194537 | 0.962678 | 0.9509592 | 0.7893555 | 0.9680063 | 1.1172072 |
| Hepatic lipase | 0.9916272 | 0.9840803 | 0.5502192 | 0.6353822 | 0.9645402 | 1.5011321 | 1.5903503 |
| Heme oxygenase | 0.9921967 | 0.9156862 | 1.2198147 | 0.935846 | 1.0112437 | 0.9836838 | 0.7229878 |
| Equilbrative nitrobenzylthioinosine- | 1.0062685 | 0.9626756 | 1.019853 | 1.1260031 | 1.2230359 | 0.9409531 | 0.8090819 |
| sensitive nucleoside transporter | | | | | | | |
| Epidermal growth factor | 1.5598979 | 0.9436674 | 0.9049939 | 1.1163325 | 1.0106174 | 0.8764173 | 0.789813 |
| c-myc | 0.9884758 | 0.9544398 | 1.1770171 | 1.000762 | 0.7601999 | 0.8303317 | 0.7356411 |
| Carbonic anhydrase III | 0.5169383 | 0.6469516 | 0.2161613 | 0.5853369 | 1.2993048 | 0.8961489 | 1.1638929 |
| Beta-actin | 1.0013711 | 1.2273529 | 0.7547822 | 0.8872546 | 0.6651123 | 1.9898698 | 1.5665321 |
| Bax (alpha) | 0.9843578 | 1.1754037 | 0.9658865 | 1.0425696 | 0.8676918 | 1.2283171 | 1.1650804 |
| Alpha 1 - inhibitor III | 0.8131611 | 0.7832114 | 0.8952138 | 0.6186565 | 0.7906568 | 1.1060873 | 1.3428322 |
| 60S ribosomal protein L6 | 0.919599 | 1.0469663 | 1.3375161 | 0.9821876 | 1.1774285 | 1.2937964 | 1.0942562 |
| Phase-1 RCT-117 | 0.878556 | 1.2696829 | 0.9639036 | 0.7564002 | 0.9792127 | 0.6846969 | 0.7727978 |
| Phase-1 RCT-102 | 1.0455929 | 1.0054173 | 0.4326966 | 0.5704886 | 0.730668 | 1.2747341 | 1.322002 |
| 3-hydroxyisobutyrate dehydrogenase | 1.0076114 | 0.9365832 | 1.1205114 | 0.959861 | 1.2833178 | 1.2081124 | 1.0479382 |
| ATPase inhibitor (rat mitochondrial IF1 | 0.824814 | 0.908179 | 1.3100915 | 1.0346922 | 1.259272 | 0.8497443 | 0.8647223 |
| protein) | | | | | | | |
| Alpha-2-macroglobulin | 1.3955147 | 0.9550351 | 1.1086873 | 0.8971185 | 0.7230533 | 1.3414469 | 1.4487567 |
| Phase-1 RCT-137 | 1.2086991 | 0.930078 | 0.9844798 | 0.9373319 | 1.2014779 | 1.5320246 | 1.5328485 |
| Phase-1 RCT 252 | 0.8240685 | 0.9214069 | 0.7293817 | 0.7126266 | 1.1433077 | 1.15678 | 1.5427232 |
| Phase-1 RCT-65 | 1.1756767 | 1.09439 | 1.0598484 | 1.2147654 | 0.9552781 | 1.2402506 | 1.0828404 |
| Liver fatty acid binding protein | 0.8323677 | 0.9996049 | 0.7306769 | 0.7608122 | 0.8945148 | 0.9993388 | 1.0276881 |
| Carbamyl phosphate synthetase I | 0.7744606 | 0.8618008 | 0.757057 | 0.7488923 | 1.2525837 | 1.0823783 | 1.7122058 |
| c-jun | 1.1467885 | 0.960893 | 0.8509684 | 1.0712705 | 0.7715727 | 1.2298893 | 0.7469471 |
| Transthyretin | 0.6881942 | 1.1540821 | 0.8122838 | 0.6442909 | 0.8193464 | 0.9507796 | 1.3673004 |
| Aflatoxin B1 aldehyde reductase | 0.8787551 | 4.0461297 | 1.9220527 | 1.4719589 | 1.7218351 | 0.9146199 | 0.8438531 |
| Phase-1 RCT-15 | 1.0634 | 1.1517425 | 1.064215 | 1.1784416 | 1.1464139 | 1.1803362 | 1.2139361 |
| Insulin-like growth factor I | 0.8585674 | 1.1589289 | 0.6507893 | 0.6364676 | 0.8717047 | 1.0127659 | 1.1894858 |
| Sodium/bile acid cotransporter | 0.6436162 | 1.0652164 | 1.7685418 | 1.3887147 | 1.7675744 | 0.8062378 | 0.853996 |
| Organic cation transporter 3 | 1.0113645 | 1.2215718 | 1.2795233 | 0.9607067 | 1.1549846 | 0.8610155 | 0.9219961 |
| Gadd153 | 0.9680713 | 0.9600416 | 0.8923781 | 1.0003737 | 0.830289 | 0.9854817 | 1.0377822 |
| Phase-1 RCT-109 | 1.0720357 | 1.269072 | 1.1660494 | 0.9145849 | 1.0070486 | 1.2585077 | 1.1634601 |
| Phase-1 RCT-88 | 0.9462617 | 0.9951761 | 0.9501799 | 0.9658228 | 0.8197932 | 0.6442019 | 0.7693523 |
| Ubiquitin conjugating enzyme (RAD 6 | 0.8596546 | 1.0171196 | 1.1927505 | 0.944146 | 1.0741978 | 0.8839144 | 0.9352797 |
| homologue) | | | | | | | |
| Glucose-regulated protein 78 | 0.9978759 | 0.9193489 | 0.9595127 | 1.1108341 | 1.1273624 | 1.3287907 | 1.7466733 |
| Ribosomal protein L13A | 1.2218935 | 1.5729314 | 1.2772131 | 0.9269971 | 1.0719514 | 0.996663 | 1.0961248 |
| Monocyte chemotactic protein receptor | 1.0364957 | 0.9605687 | 1.2497525 | 0.9714974 | 1.0991983 | 0.8782294 | 0.9790759 |
| (CCR2) | | | | | | | |
| Fas antigen | 0.9228005 | 1.0511218 | 1.02494 | 0.9582462 | 0.8062391 | 1.5606765 | 1.2360479 |
| Calpactin I heavy chain | 1.2490076 | 1.1615725 | 0.8451143 | 1.0772192 | 0.9711416 | 0.9331496 | 0.8750598 |
| Uncoupling protein 2 | 1.0701581 | 1.1689075 | 0.9360005 | 0.9242452 | 0.7995296 | 1.0480435 | 0.997496 |
| Phase-1 RCT-168 | 0.9422062 | 0.9140951 | 0.8567884 | 0.9469474 | 1.0212011 | 1.1483575 | 1.0962225 |
| Phase-1 RCT-154 | 1.0047576 | 1.0600618 | 1.0376146 | 0.9975066 | 1.1128929 | 0.6096191 | 0.9438074 |
| Superoxide dismutase Mn | 1.2432184 | 1.6476307 | 1.5030962 | 1.1641678 | 1.1524429 | 1.3086507 | 1.04525 |
| Phase-1 RCT-214 | 0.9527875 | 0.9204301 | 0.8425432 | 1.0543047 | 1.0770663 | 1.4979415 | 2.3256223 |
| Phase-1 RCT-225 | 1.2174127 | 0.9831747 | 1.7392437 | 2.1131594 | 1.3423799 | 0.9381729 | 1.236395 |
| Phase-1 RCT-181 | 1.1220124 | 0.9642751 | 0.8080831 | 0.8735644 | 1.0180587 | 0.8815234 | 1.0245229 |
| Nucleoside diphosphate kinase beta | 1.0558496 | 1.2792239 | 0.7941501 | 1.0832835 | 1.2344192 | 1.1649014 | 1.2285272 |
| isoform | | | | | | | |
| Extracellular-signal-regulated kinase 1 | 0.5709716 | 0.6907105 | 0.3225279 | 0.6977566 | 1.1630868 | 0.9517077 | 1.1540576 |
| {Ribosomal protein L6} | 1.1919546 | 1.4806808 | 1.3701717 | 0.9989895 | 1.2543863 | 1.3254699 | 1.2997738 |
| Aldehyde dehydrogenase, microsomal | 0.9563665 | 1.2111809 | 0.9944133 | 0.9103261 | 0.9179897 | 1.3297373 | 1.1775002 |
| p53 | 0.9565139 | 0.9802603 | 1.0004919 | 0.9028463 | 0.9886983 | 1.3718901 | 1.5228194 |
| Phase-1 RCT-239 | 1.0702546 | 1.1132203 | 0.817179 | 1.0907521 | 0.8635912 | 0.7349997 | 0.770281 |
| Macrophage inflammatory protein-2 alpha | 0.9790421 | 0.9847329 | 1.125653 | 1.0495106 | 0.7755672 | 0.9853954 | 1.1093072 |
| IgE binding protein | 1.0041564 | 0.9184637 | 1.1317782 | 0.9805388 | 1.0442654 | 0.8431575 | 0.7832122 |
| Phase-1 RCT-205 | 0.9609811 | 0.9060189 | 1.0633568 | 1.064425 | 1.0567652 | 0.8926227 | 0.9525448 |
| Melanoma-associated antigen ME491 | 1.107526 | 0.869632 | 0.8674341 | 1.1272734 | 0.7974557 | 0.7924301 | 0.7988351 |
| Phase-1 RCT-242 | 1.0072429 | 0.9629999 | 0.9170792 | 1.0137064 | 0.8167439 | 0.6771247 | 0.7451775 |
| Enolase alpha | 0.9665234 | 1.1041591 | 0.9285306 | 0.996917 | 1.0612432 | 1.1908118 | 1.2814697 |
| Thymosin beta-10 | 1.0507609 | 1.218369 | 1.1293613 | 0.9054156 | 0.9386586 | 1.5576366 | 1.2378267 |
| Cofilin | 0.920177 | 1.0581415 | 0.9782915 | 0.8712325 | 1.0250045 | 1.0805206 | 1.1256176 |
| Carbonyl reductase | 0.990534 | 0.959385 | 1.1054786 | 1.0237094 | 0.9666029 | 0.9855207 | 1.1195915 |
| MAP kinase kinase | 1.0682081 | 1.0474654 | 0.9393023 | 1.0700102 | 0.9289074 | 1.1106898 | 0.9838817 |
| Phase-1 RCT-162 | 1.2283758 | 1.4276313 | 1.1163574 | 0.9613192 | 1.1278867 | 0.8837471 | 0.9914696 |
| Phase-1 RCT-72 | 0.9899353 | 0.9517062 | 0.8425267 | 1.0421966 | 0.920265 | 0.7625921 | 0.7456297 |
| Ribosomal protein S8 | 1.4045637 | 1.7173852 | 1.5392064 | 1.0530316 | 1.3560612 | 1.2051417 | 1.2571408 |
| Proliferating cell nuclear antigen gene | 0.9915175 | 0.9149498 | 0.904964 | 0.9277663 | 0.9369111 | 0.8519644 | 0.8553965 |
| Phase-1 RCT-192 | 1.0741752 | 1.281383 | 0.9390487 | 1.004074 | 1.1469207 | 0.9492441 | 1.0434508 |
| IkB-a | 0.8701468 | 1.0878115 | 1.1292201 | 0.9829286 | 1.1370168 | 0.9275752 | 0.9655762 |
| Endogenous retroviral sequence, 5′ and | 0.9853415 | 0.8941175 | 1.0212339 | 1.6538879 | 1.4996394 | 0.9137799 | 1.2908863 |
| 3′ LTR | | | | | | | |
| Phase-1 RCT-37 | 1.0841764 | 1.2106717 | 1.0982636 | 0.938845 | 1.1379498 | 0.9750233 | 0.9268447 |
| Matrix metalloproteinase-1 | 1.3279779 | 1.2292411 | 1.424628 | 0.9722884 | 1.1686628 | 1.0763032 | 1.1874627 |
| Cyclin G | 0.966465 | 1.0430251 | 0.9929118 | 1.1240302 | 1.1363571 | 0.8723437 | 0.9866107 |
| DNA polymerase beta | 0.9544553 | 0.9559873 | 1.0697848 | 1.0466899 | 1.2621326 | 0.9162179 | 1.1857452 |
| Phase-1 RCT-127 | 0.9143099 | 1.0351822 | 1.0427216 | 0.8638739 | 0.9552475 | 0.7181895 | 0.8335391 |
| Multidrug resistant protein-2 | 1.3038982 | 1.1755364 | 1.8075417 | 1.4280765 | 0.9500036 | 1.1236757 | 0.9125143 |
| Ribosomal protein S9 | 0.7855111 | 1.1152974 | 1.091868 | 0.9348629 | 1.156297 | 1.2270125 | 1.7409263 |
| Phase-1 RCT-12 | 1.1247871 | 1.1875986 | 0.8599771 | 1.0096121 | 0.9293074 | 1.0116947 | 0.9954492 |
| Beta-tubulin, class I | 1.2848592 | 1.6821 | 0.7728321 | 1.2201084 | 1.1359999 | 1.6063566 | 1.6603278 |
| Argininosuccinate lyase | 0.9435762 | 1.5382729 | 1.0327706 | 0.8564189 | 0.9743807 | 1.4231238 | 1.6798581 |
| Elongation factor-1 alpha | 1.313477 | 1.2903754 | 1.6832963 | 1.0348657 | 1.1669741 | 1.2259861 | 1.2377819 |
| Phase-1 RCT-55 | 0.9746494 | 0.9255465 | 0.9228968 | 1.1036797 | 0.877193 | 0.7037122 | 0.6634403 |
| Macrophage inflammatory protein-1 alpha | 0.9467006 | 0.9346252 | 1.010685 | 1.0178694 | 0.8506803 | 0.8996736 | 0.968917 |
| c-H-ras | 1.104404 | 1.2867297 | 1.1091162 | 1.0370576 | 1.0000762 | 1.1226699 | 1.1201024 |
| Multidrug resistant protein-1 | 1.1263776 | 1.1982965 | 1.5288286 | 1.2348312 | 0.9802235 | 1.0688647 | 0.9309531 |
| T-cell cyclophilin | 1.0441507 | 0.8825584 | 1.076737 | 0.9888333 | 1.2858677 | 0.9594932 | 1.1880453 |
| Phase-1 RCT-185 | 0.7975301 | 1.0195429 | 0.8927769 | 0.7917763 | 0.9982331 | 1.1699415 | 1.0961187 |
| 25-DX | 0.8781971 | 0.9413058 | 1.5600784 | 1.040287 | 1.2409428 | 1.1315013 | 1.0382785 |
| Ref-1 | 1.1059946 | 1.2715389 | 1.5023463 | 1.5066134 | 1.5721827 | 0.9470246 | 1.1321822 |
| Apolipoprotein CIII | 0.6405088 | 0.9026303 | 1.256718 | 0.9488789 | 1.2317374 | 1.260566 | 1.1291071 |
| NADPH quinone oxidoreductase-1 (DT- | 1.1300856 | 1.706858 | 3.7278683 | 3.0025556 | 3.550085 | 1.1209266 | 1.1346557 |
| diaphorase) | | | | | | | |
| Pyruvate kinase, muscle | 1.079561 | 1.0017515 | 1.1539986 | 1.0684897 | 0.9974641 | 1.4432871 | 1.2499297 |
| | BUS | CAD | CAD | CAD | CAR | CAR | CAR |
| Compound-Dose (2) | 14 | 1 | 1 | 1 | 16 | 16 | 16 |
| Animal Number (3) | 1746 | 244 | 245 | 246 | 1854 | 1855 | 1856 |
| Liver Toxicity Necrosis Classification (4) | no | no | no | no | no | no | no |
| Gene Name (5) | | | | | | | |
| Phase-1 RCT-78 | 1.1592139 | 1.0255255 | 1.037231 | 0.9999999 | 1.094995 | 1.0938365 | 1.0033218 |
| Gamma-actin, cytoplasmic | 0.6641409 | 0.8666718 | 1.111525 | 0.8218571 | 1 | 0.8829377 | 0.7036943 |
| Matrin F/G | 1.1215018 | 1.3330951 | 1.7573369 | 1.5132957 | 0.715763 | 0.7637605 | 0.7477419 |
| Zinc finger protein | 1.6728373 | 0.9664831 | 0.9074502 | 0.9915353 | 0.9499301 | 0.9958291 | 1.0325587 |
| Cathepsin L, sequence 2 | 1.2030499 | 0.6396752 | 1.1254956 | 0.8209748 | 0.8658587 | 0.9574473 | 1.2842411 |
| Gadd45 | 0.9821052 | 0.9431784 | 0.9677562 | 1.109791 | 0.9853353 | 0.9105629 | 1.0144325 |
| Phase-1 RCT-144 | 0.9512708 | 1.1149272 | 1.0604142 | 1.0921812 | 0.9258371 | 0.9570468 | 0.9097343 |
| Phase-1 RCT-145 | 0.980446 | 1.1531684 | 0.9501364 | 0.9674025 | 0.9431939 | 1.0105226 | 0.9734493 |
| Phase-1 RCT-50 | 0.9755142 | 1.0525353 | 0.9758069 | 0.9306669 | 1.1225919 | 1.0972033 | 0.9559513 |
| Phase-1 RCT-92 | 0.9162518 | 1.0773294 | 0.8797283 | 0.9298616 | 0.772517 | 0.7920139 | 0.7614585 |
| Phase-1 RCT-89 | 1.0300634 | 0.6329765 | 1.1136876 | 1.0348824 | 0.7646036 | 0.8133516 | 0.8981553 |
| 14-3-3 zeta | 0.889012 | 0.7335754 | 0.9097308 | 1.0473813 | 1.509641 | 1.5396113 | 1.8436518 |
| Dynamin-1 (D100) | 0.963396 | 1.1475768 | 0.9417752 | 1.1772211 | 0.9335519 | 0.8303874 | 0.7918361 |
| Insulin-like growth factor binding protein 1 | 0.9242942 | 0.7882763 | 0.7984562 | 1.2245544 | 1.1220509 | 0.8957977 | 1.1789569 |
| L-gulono-gamma-lactone oxidase | 1.1687533 | 0.7465334 | 0.6365684 | 1.5977325 | 0.4994488 | 0.686313 | 0.6093361 |
| Ornithine decarboxylase | 1.0427198 | 1.0727961 | 0.9122623 | 1.009704 | 1.7512414 | 1.8125535 | 1.8936803 |
| PAR interacting protein | 1.0018455 | 1.1052963 | 1.0979998 | 1.1271478 | 0.9763326 | 0.9918281 | 0.9517729 |
| Phase-1 RCT-128 | 1.0042634 | 0.988745 | 0.9209751 | 1.5306062 | 0.8426918 | 0.6982569 | 0.7098169 |
| Phase-1 RCT-180 | 1.0236653 | 0.8059568 | 0.8250722 | 0.9902005 | 0.9313951 | 1.0926203 | 0.927073 |
| Phase-1 RCT-182 | 1.0348463 | 0.6793788 | 0.880818 | 0.6001697 | 0.8692953 | 0.7930051 | 0.9749695 |
| Phase-1 RCT-207 | 0.9828317 | 1.1221644 | 0.8647441 | 0.9696736 | 1.7816856 | 1.4003307 | 1.2896867 |
| Phase-1 RCT-213 | 0.9700677 | 1.1356136 | 1.0875949 | 0.9467157 | 1.0131752 | 0.9960768 | 0.9872913 |
| Phase-1 RCT-256 | 1.0485976 | 0.992386 | 1.1650034 | 2.0995169 | 0.6144337 | 0.6324272 | 0.5620555 |
| Phase-1 RCT-258 | 0.9848343 | 1.2037048 | 1.0627469 | 0.9989098 | 0.9807609 | 0.9875321 | 0.9803354 |
| Phase-1 RCT-264 | 1.042209 | 0.6900498 | 0.5835168 | 0.6347438 | 0.7431545 | 0.9155045 | 0.8448186 |
| Phase-1 RCT-271 | 1.2406162 | 0.754853 | 0.7788186 | 1.4595602 | 0.8055501 | 0.7172052 | 0.7672992 |
| Phase-1 RCT-288 | 1.0287255 | 0.8771248 | 0.8734779 | 0.816827 | 0.6741667 | 0.6263041 | 0.7370434 |
| Phase-1 RCT-38 | 1.0893149 | 0.8614677 | 1.1112626 | 1.9753243 | 0.6005413 | 0.6170121 | 0.5720821 |
| Phase-1 RCT-39 | 1.0023216 | 0.8664179 | 1.0967039 | 1.0310755 | 1.1335663 | 1.1751024 | 1.1050638 |
| Phase-1 RCT-68 | 0.9694255 | 1.1183589 | 1.113307 | 1.0495281 | 1.2174321 | 1.1606094 | 1.2079067 |
| Phase-1 RCT-33 | 1.2316489 | 0.8145452 | 0.9519779 | 2.0443347 | 0.6376173 | 0.9493131 | 0.9545052 |
| Phase-1 RCT-36 | 1.0313365 | 1.036553 | 1.0543965 | 1.3902068 | 0.7534571 | 0.9879119 | 0.9849631 |
| 17-beta hydroxysteroid dehydrogenase, | 1.8894733 | 0.5299866 | 0.8516602 | 0.6702575 | 1.163904 | 1.3181926 | 2.9710505 |
| type 2 | | | | | | | |
| Senescence marker protein-30 | 0.9724526 | 0.8535687 | 1.0588164 | 0.8261873 | 0.8079969 | 1.0894308 | 1.0861651 |
| Ribosomal protein S17 | 1.0604584 | 1.3687737 | 1.4031212 | 1.4624434 | 0.7203303 | 0.6330063 | 0.7241964 |
| Phase-1 RCT-83 | 0.8371213 | 0.8690555 | 1.0913544 | 0.9754183 | 0.9055849 | 0.9438733 | 0.8014066 |
| Phase-1 RCT-49 | 0.9800095 | 1.3326769 | 1.0930429 | 1.3702456 | 0.855736 | 0.8570986 | 0.8813155 |
| Phase-1 RCT-48 | 1.0121841 | 0.8351429 | 0.8958237 | 0.8486352 | 0.7116521 | 0.7486877 | 0.6821266 |
| Phase-1 RCT-40 | 1.1394655 | 0.7982522 | 1.1178788 | 0.896129 | 0.8211058 | 0.8252744 | 0.8487489 |
| Phase-1 RCT-296 | 1.1817225 | 0.5929669 | 0.6353468 | 0.6629982 | 0.4277205 | 0.647689 | 0.5303079 |
| Phase-1 RCT-295 | 1.1130731 | 1.1257282 | 1.1251417 | 1.3872546 | 1.1439148 | 1.1804931 | 1.2091378 |
| Phase-1 RCT-291 | 1.0985883 | 0.7386515 | 0.9563837 | 0.8441966 | 0.8217449 | 0.7856808 | 0.8132479 |
| Phase-1 RCT-270 | 0.9655659 | 0.7355066 | 0.7515767 | 0.6579805 | 0.7251484 | 0.7380418 | 0.6708258 |
| Phase-1 RCT-241 | 0.9756365 | 1.1733422 | 1.051228 | 1.0670377 | 0.9534594 | 1.0059595 | 0.9790716 |
| Phase-1 RCT-191 | 0.9850165 | 0.9112554 | 0.9419333 | 0.9386287 | 1.1062262 | 1.1899693 | 1.1308348 |
| Phase-1 RCT-189 | 1.081937 | 0.8649894 | 0.8595485 | 1.1802347 | 0.7720637 | 0.8566768 | 0.9018569 |
| Phase-1 RCT-179 | 1.1413467 | 0.8119224 | 0.8146477 | 0.9005458 | 1.0859321 | 1.0291111 | 0.9461888 |
| Phase-1 RCT-152 | 1.1412129 | 1.1489742 | 1.3356917 | 1.5576998 | 0.8305301 | 0.8178273 | 0.8674403 |
| Phase-1 RCT-123 | 1.0412254 | 1.302592 | 1.1165439 | 0.9683829 | 0.9715666 | 0.9894803 | 1.0204797 |
| Paraoxonase 1 | 0.9785816 | 0.6680378 | 0.9188719 | 0.6333563 | 0.5920301 | 0.746118 | 0.6769486 |
| Organic anion transporter 3 | 1.4680489 | 0.7496769 | 0.7592143 | 0.921734 | 1.2942573 | 1.218386 | 1.2057085 |
| N-hydroxy-2-acetylaminofluorene | 1.2692161 | 0.6147558 | 1.0025032 | 0.7438684 | 0.7341777 | 0.7940046 | 0.7133854 |
| sulfotransferase (ST1C1) | | | | | | | |
| Integrin beta1 | 0.9992426 | 1.0922253 | 1.0896685 | 1.2927763 | 1.0638644 | 1.04303 | 1.1443281 |
| Insulin-like growth factor binding protein 3 | 1.0734738 | 0.5480973 | 0.8539287 | 0.8597077 | 0.694198 | 0.7061638 | 0.8060978 |
| ID-1 | 0.8737182 | 0.8101889 | 0.8764965 | 0.9313932 | 1.5675051 | 1.0410495 | 1.6455994 |
| Hepatic lipase | 0.9803594 | 0.7948307 | 0.870493 | 0.9710074 | 0.3841917 | 0.5046774 | 0.3899094 |
| Heme oxygenase | 1.1051673 | 1.1534878 | 1.2350689 | 1.2944899 | 0.8273408 | 1.041064 | 0.7933779 |
| Equilbrative nitrobenzylthioinosine- | 1.0728408 | 0.7312239 | 1.0191307 | 0.7873164 | 0.7709897 | 0.7927386 | 0.7217221 |
| sensitive nucleoside transporter | | | | | | | |
| Epidermal growth factor | 1.0646371 | 1.2324198 | 1.3430192 | 1.0322853 | 0.9319288 | 0.8783072 | 0.9024622 |
| c-myc | 1.0009671 | 0.8956041 | 0.8340899 | 1.0036646 | 1.1224029 | 1.085955 | 1.0484827 |
| Carbonic anhydrase III | 1.0423691 | 0.8636505 | 0.5319681 | 0.7357204 | 1.1452497 | 1.6406586 | 0.7793642 |
| Beta-actin | 0.4525807 | 0.6335506 | 1.0248864 | 0.8814244 | 2.2156713 | 1.9706372 | 1.975018 |
| Bax (alpha) | 0.9752275 | 0.7871565 | 0.6834517 | 0.8658935 | 1.4134742 | 1.2759356 | 1.3882053 |
| Alpha 1 - inhibitor III | 0.874452 | 0.5270844 | 0.4597887 | 0.4511813 | 0.5728379 | 0.6374714 | 0.7406561 |
| 60S ribosomal protein L6 | 1.0766976 | 0.9467462 | 0.9731745 | 1.5153639 | 0.8710781 | 0.8295908 | 0.9655191 |
| Phase-1 RCT-117 | 0.9934201 | 1.2419946 | 1.0635422 | 1.5562509 | 1.1237067 | 0.8045632 | 0.9733849 |
| Phase-1 RCT-102 | 1.1973782 | 0.5822538 | 0.709641 | 0.5626305 | 0.4887968 | 0.4997405 | 0.4448672 |
| 3-hydroxyisobutyrate dehydrogenase | 1.1927274 | 0.8547316 | 2.2619987 | 0.958683 | 0.8012401 | 0.8007858 | 0.8965268 |
| ATPase inhibitor (rat mitochondrial IF1 | 0.9305126 | 1.0962267 | 1.2821177 | 2.0782266 | 0.4888459 | 0.5615355 | 0.5625629 |
| protein) | | | | | | | |
| Alpha-2-macroglobulin | 0.913617 | 0.6832901 | 0.3540675 | 0.4660623 | 0.6272326 | 0.6523749 | 0.8874352 |
| Phase-1 RCT-137 | 1.2714103 | 0.8471698 | 0.9600238 | 1.0444139 | 0.5969433 | 0.6191209 | 0.6511688 |
| Phase-1 RCT 252 | 1.0912942 | 0.7861117 | 0.875398 | 1.4913071 | 0.5085026 | 0.6548359 | 0.5197458 |
| Phase-1 RCT-65 | 0.9326206 | 0.7236796 | 0.9002927 | 0.7596491 | 1.5458307 | 1.5591791 | 1.6128478 |
| Liver fatty acid binding protein | 0.8224365 | 0.6949953 | 0.8177401 | 1.6706816 | 0.4792838 | 0.5505199 | 0.6268606 |
| Carbamyl phosphate synthetase I | 0.9929828 | 0.7354301 | 0.9489195 | 1.4618624 | 0.4964423 | 0.6349035 | 0.5175007 |
| c-jun | 1.0752187 | 0.8397844 | 0.9237731 | 0.8603768 | 1.8101414 | 1.393942 | 1.7459549 |
| Transthyretin | 0.8813847 | 0.6311055 | 0.5103184 | 0.5799006 | 0.6311048 | 0.7395544 | 0.7257287 |
| Aflatoxin B1 aldehyde reductase | 1.0021297 | 0.8639818 | 1.0473567 | 1.395244 | 1.0457866 | 1.3519762 | 1.0494235 |
| Phase-1 RCT-15 | 1.0378957 | 0.8841616 | 0.8426145 | 0.9099756 | 1.3704808 | 1.4056506 | 1.2723596 |
| Insulin-like growth factor I | 0.7864628 | 0.5257295 | 0.8004057 | 0.8192999 | 0.6362239 | 0.6363815 | 0.660366 |
| Sodium/bile acid cotransporter | 1.1589404 | 0.4929412 | 0.8253326 | 1.1542131 | 0.8335502 | 0.8425593 | 1.1373435 |
| Organic cation transporter 3 | 1.0308381 | 1.0415475 | 0.9418556 | 1.4128814 | 0.9542267 | 0.8987916 | 0.9719366 |
| Gadd153 | 1.0283177 | 0.71057 | 0.7239225 | 0.9012818 | 1.5213019 | 1.3283073 | 1.3606365 |
| Phase-1 RCT-109 | 1.0631162 | 1.6612374 | 1.5067303 | 2.16972 | 0.9878065 | 0.828809 | 1.0014424 |
| Phase-1 RCT-88 | 0.9494334 | 1.3276424 | 0.9645208 | 0.9035673 | 1.0077969 | 0.9875003 | 1.0005672 |
| Ubiquitin conjugating enzyme (RAD 6 | 0.9690218 | 1.0268016 | 0.9754465 | 1.0077807 | 1.0897999 | 1.1350448 | 1.2170075 |
| homologue) | | | | | | | |
| Glucose-regulated protein 78 | 0.9301953 | 1.7200215 | 1.3762052 | 1.6751324 | 1.1721681 | 1.23007 | 1.3411301 |
| Ribosomal protein L13A | 0.9507009 | 1.1807262 | 1.0965607 | 1.209073 | 1.0612388 | 0.8486543 | 1.1048334 |
| Monocyte chemotactic protein receptor | 0.9598336 | 0.9598095 | 0.9292723 | 1.0711172 | 1.1801386 | 1.0922614 | 1.0605185 |
| (CCR2) | | | | | | | |
| Fas antigen | 0.9835884 | 1.0658228 | 0.9866289 | 1.1449132 | 1.4370484 | 1.2802213 | 1.6670308 |
| Calpactin I heavy chain | 0.980328 | 1.236004 | 1.1587111 | 1.4105617 | 1.253714 | 1.2858303 | 1.3621274 |
| Uncoupling protein 2 | 0.8878852 | 1.1613408 | 0.9100074 | 1.1839678 | 1.0606512 | 1.0028626 | 1.1287041 |
| Phase-1 RCT-168 | 1.1304932 | 1.1725396 | 1.0275257 | 1.3379147 | 0.7474626 | 0.8162275 | 0.8237702 |
| Phase-1 RCT-154 | 1.0015068 | 1.2774769 | 1.0318981 | 1.025087 | 1.0600232 | 1.0306317 | 1.1097126 |
| Superoxide dismutase Mn | 1.1474055 | 1.0890474 | 1.3737698 | 1.2037665 | 1.1438038 | 1.0886627 | 1.214644 |
| Phase-1 RCT-214 | 1.1197827 | 1.215907 | 0.9095075 | 1.2023371 | 0.9574882 | 1.1108873 | 0.9547927 |
| Phase-1 RCT-225 | 1.1933484 | 0.960947 | 0.9440668 | 1.0044096 | 2.1085238 | 1.9944754 | 1.0219359 |
| Phase-1 RCT-181 | 1.0217246 | 1.1124297 | 0.9637182 | 1.0777204 | 0.7935016 | 0.7499104 | 0.990494 |
| Nucleoside diphosphate kinase beta | 1.2170374 | 1.4073727 | 1.4542128 | 1.516178 | 1.0686402 | 1.0967051 | 1.1948473 |
| isoform | | | | | | | |
| Extracellular-signal-regulated kinase 1 | 0.9786702 | 0.9065967 | 0.8098788 | 0.8598487 | 1.284846 | 1.6813183 | 0.8582825 |
| {Ribosomal protein L6} | 1.01772 | 1.1154768 | 1.2342839 | 1.4355958 | 0.7872725 | 0.7986205 | 0.8174483 |
| Aldehyde dehydrogenase, microsomal | 1.1683462 | 0.6866641 | 0.7849079 | 0.9846404 | 0.9889172 | 0.933796 | 0.9089764 |
| p53 | 0.943315 | 0.8390218 | 0.8548355 | 0.9701474 | 0.9981423 | 0.9538426 | 0.8721052 |
| Phase-1 RCT-239 | 0.9328992 | 0.9949058 | 0.8875659 | 0.9455234 | 1.1297386 | 1.073292 | 0.9630342 |
| Macrophage inflammatory protein-2 alpha | 1.0592467 | 1.0709677 | 0.908618 | 1.0554452 | 1.4427378 | 1.3881137 | 1.5386143 |
| IgE binding protein | 1.0193554 | 1.5480868 | 1.7091323 | 1.5237262 | 0.9268111 | 0.9124182 | 0.933777 |
| Phase-1 RCT-205 | 1.002539 | 1.2277259 | 1.3401694 | 1.0983773 | 0.8433959 | 0.8338369 | 0.8478423 |
| Melanoma-associated antigen ME491 | 0.9487007 | 0.9692444 | 0.9559595 | 0.9000151 | 0.9739546 | 1.0057329 | 1.2318835 |
| Phase-1 RCT-242 | 0.8567058 | 1.4282796 | 1.1295344 | 1.0839249 | 1.1610334 | 1.0551423 | 0.9816344 |
| Enolase alpha | 1.3597603 | 2.4880288 | 2.1044474 | 1.5376272 | 1.3292711 | 0.9658459 | 0.9293606 |
| Thymosin beta-10 | 1 | 1.3059582 | 0.9985989 | 1.5459284 | 1.0284693 | 0.8843569 | 1.0969594 |
| Cofilin | 1.001616 | 1.0721028 | 1.1750342 | 1.0561879 | 0.8405904 | 0.8904647 | 0.9566366 |
| Carbonyl reductase | 1.053322 | 0.6911106 | 0.6986458 | 0.7612382 | 1.125839 | 1.1323282 | 1.118233 |
| MAP kinase kinase | 1.0395789 | 0.7323237 | 0.8604 | 0.8798033 | 0.7863244 | 0.7529784 | 0.9128637 |
| Phase-1 RCT-162 | 1.0343021 | 1.1153677 | 1.1107585 | 1.1923879 | 0.9295715 | 0.9309032 | 0.9847314 |
| Phase-1 RCT-72 | 0.9563429 | 1.2187343 | 1.2369455 | 1.0925539 | 0.9602623 | 0.9724191 | 0.8551151 |
| Ribosomal protein S8 | 1.1210288 | 1.1720573 | 1.2804202 | 1.500759 | 0.8781387 | 0.7865298 | 0.8907942 |
| Proliferating cell nuclear antigen gene | 0.9836609 | 0.9863698 | 0.8708534 | 0.9429366 | 1.0233011 | 1.0262103 | 0.9510356 |
| Phase-1 RCT-192 | 1.0452284 | 1.0989672 | 1.0036055 | 1.4271226 | 1.0091796 | 0.8989343 | 1.1264547 |
| IkB-a | 0.9161865 | 1.1221476 | 1.2289935 | 1.2589473 | 0.8749204 | 0.7559072 | 0.8081187 |
| Endogenous retroviral sequence, 5′ and | 0.8435459 | 0.5731911 | 0.8326342 | 0.7410591 | 1.5978316 | 1.1344993 | 1.4405288 |
| 3′ LTR | | | | | | | |
| Phase-1 RCT-37 | 1.053153 | 1.223352 | 1.2522728 | 1.2397865 | 0.9342423 | 0.8935678 | 0.9267863 |
| Matrix metalloproteinase-1 | 0.8445419 | 0.7758035 | 0.9761224 | 0.9065605 | 1.189299 | 1.0646465 | 1.1838274 |
| Cyclin G | 0.9710991 | 1.827208 | 1.1988646 | 1.1512 | 2.8839307 | 2.176605 | 2.3467317 |
| DNA polymerase beta | 0.9208665 | 1.2422088 | 1.1368307 | 1.226051 | 0.8252841 | 0.8986716 | 0.9060223 |
| Phase-1 RCT-127 | 0.9909289 | 1.25508 | 1.130625 | 1.2442247 | 0.9875379 | 0.9988158 | 0.9412854 |
| Multidrug resistant protein-2 | 0.9161109 | 0.6826693 | 0.8683397 | 0.7558366 | 1.6322926 | 1.3965453 | 1.8623515 |
| Ribosomal protein S9 | 1.1375246 | 1.096867 | 0.8357224 | 0.912419 | 0.9680114 | 0.9740847 | 1.0761544 |
| Phase-1 RCT-12 | 0.9452896 | 0.8918211 | 0.9285766 | 1.2656366 | 1.3455998 | 1.1222386 | 1.2873701 |
| Beta-tubulin, class I | 0.8051437 | 0.9105088 | 1.0500515 | 1.7913461 | 1.2118707 | 0.939521 | 1.0667461 |
| Argininosuccinate lyase | 1.1152531 | 0.8062986 | 0.7175926 | 0.8192643 | 0.9036452 | 1.0233389 | 1.0559356 |
| Elongation factor-1 alpha | 1.0568441 | 0.7354469 | 0.9957939 | 0.8099166 | 0.8684222 | 0.8406044 | 1.0197695 |
| Phase-1 RCT-55 | 0.9369386 | 1.1539267 | 1.1409793 | 1.0249784 | 1.1441793 | 1.029413 | 0.9226376 |
| Macrophage inflammatory protein-1 alpha | 0.9221086 | 0.9112554 | 0.8646643 | 0.8426807 | 1.1816934 | 1.1296769 | 1.095676 |
| c-H-ras | 1.0224457 | 0.6681131 | 0.8430361 | 1.1584265 | 1.2512568 | 1.2708756 | 1.3148922 |
| Multidrug resistant protein-1 | 0.8738104 | 0.7425667 | 0.7753388 | 0.681693 | 1.7290249 | 1.5513285 | 2.077706 |
| T-cell cyclophilin | 1.0234853 | 1.2490038 | 1.1140512 | 1.2764155 | 0.7704579 | 0.7977839 | 0.8579325 |
| Phase-1 RCT-185 | 1.070547 | 0.8489767 | 1.0078487 | 0.8494245 | 0.6739773 | 0.7554829 | 0.7696208 |
| 25-DX | 1.1153094 | 0.9824517 | 0.9579042 | 1.5592701 | 1.027093 | 1.0700144 | 1.0955297 |
| Ref-1 | 1.0505832 | 1.0907452 | 1.0409725 | 1.0307381 | 1.4107288 | 1.4345379 | 1.3571587 |
| Apolipoprotein CIII | 0.990823 | 1.0819147 | 0.746855 | 0.8102197 | 0.8401556 | 0.8283916 | 0.8595325 |
| NADPH quinone oxidoreductase-1 (DT- | 0.8942895 | 0.9439951 | 1.2331557 | 0.8850905 | 1.4758272 | 1.4413787 | 1.6117979 |
| diaphorase) | | | | | | | |
| Pyruvate kinase, muscle | 1.1480552 | 1.0774521 | 1.0461551 | 1.4214342 | 0.8044874 | 0.8092774 | 0.7775644 |
| | CHCL3 | CHCL3 | CHCL3 | CHCL3 | CHCL3 | CHCL3 | CHLOR |
| Compound-Dose (2) | 250 | 250 | 250 | 500 | 500 | 500 | 8 |
| Animal Number (3) | 1624 | 1625 | 1626 | 2354 | 2355 | 2356 | 44 |
| Liver Toxicity Necrosis Classification (4) | no | no | no | no | no | no | no |
| Gene Name (5) | | | | | | | |
| Phase-1 RCT-78 | 0.6371437 | 0.8716484 | 0.7244269 | 0.956264 | 0.8040589 | 0.7710564 | 1.0628641 |
| Gamma-actin, cytoplasmic | 1.3966328 | 2.0080492 | 4.828458 | 1.6725786 | 3.1347418 | 2.8372366 | 1.0166708 |
| Matrin F/G | 0.3705433 | 0.8586965 | 0.4019924 | 0.9700947 | 0.6012951 | 0.4644135 | 1.5895839 |
| Zinc finger protein | 5.140148 | 1.0743979 | 2.2721379 | 1.4667269 | 2.5985198 | 3.465775 | 0.9695364 |
| Cathepsin L, sequence 2 | 1.1611915 | 1.8056684 | 5.1170006 | 2.444574 | 3.539196 | 2.7840476 | 1.4868618 |
| Gadd45 | 2.4178674 | 0.8442409 | 1.6548092 | 1.0283352 | 1.6095598 | 2.0979147 | 0.8886619 |
| Phase-1 RCT-144 | 2.334774 | 1.151626 | 1.4679184 | 1.0791944 | 1.841862 | 2.381963 | 0.7559324 |
| Phase-1 RCT-145 | 1.9835876 | 1.3002892 | 1.9688524 | 1.2414786 | 1.8897154 | 2.0073514 | 1.0311143 |
| Phase-1 RCT-50 | 2.9030688 | 1.0186405 | 1.5215645 | 1.0288959 | 1.4064637 | 2.2622712 | 1.0640138 |
| Phase-1 RCT-92 | 0.4892659 | 1.028068 | 0.4621187 | 0.7895095 | 0.574755 | 0.4396825 | 1.0119021 |
| Phase-1 RCT-89 | 0.4317204 | 0.766854 | 0.4762353 | 0.8087208 | 0.532439 | 0.4468516 | 0.8273964 |
| 14-3-3 zeta | 1.1644213 | 1.3859208 | 1.9416331 | 1.212394 | 2.0705886 | 1.3541808 | 1.3820266 |
| Dynamin-1 (D100) | 0.6809692 | 0.7518817 | 0.6401772 | 0.9444392 | 0.8206269 | 0.7895032 | 1.2720165 |
| Insulin-like growth factor binding protein 1 | 4.683118 | 1.3476887 | 2.9222531 | 1.3186442 | 1.984494 | 2.4675303 | 1.6317682 |
| L-gulono-gamma-lactone oxidase | 0.2773432 | 0.5586789 | 0.2578902 | 0.3634121 | 0.2624145 | 0.1553042 | 1.2909374 |
| Ornithine decarboxylase | 1.5195441 | 1.5541869 | 1.4205314 | 1.1245701 | 4.25675 | 0.8829816 | 1.6334985 |
| PAR interacting protein | 1.8909643 | 1.2167732 | 1.3892258 | 1.0470545 | 1.5827543 | 1.8112622 | 0.7598445 |
| Phase-1 RCT-128 | 0.4121503 | 0.805028 | 0.3623458 | 0.9482043 | 0.731676 | 0.7153035 | 1.123019 |
| Phase-1 RCT-180 | 1.5412409 | 1.4006307 | 1.5051446 | 1.2572634 | 1.8287534 | 1.7213008 | 0.9861962 |
| Phase-1 RCT-182 | 0.519241 | 0.8219003 | 0.6963694 | 1.038103 | 0.6816375 | 0.8025111 | 0.7886264 |
| Phase-1 RCT-207 | 1.2676375 | 1.1690223 | 2.3826344 | 1.4618077 | 2.2590778 | 2.4690695 | 1.276991 |
| Phase-1 RCT-213 | 1.5956331 | 1.3910927 | 1.9398804 | 1.2954518 | 1.8307228 | 1.6998764 | 1.4765493 |
| Phase-1 RCT-256 | 0.3308325 | 0.7697096 | 0.3920282 | 0.6655492 | 0.3803094 | 0.303441 | 1.7338086 |
| Phase-1 RCT-258 | 1.6582546 | 1.3462856 | 1.7928323 | 1.4302068 | 1.5394285 | 1.6584333 | 1.0626582 |
| Phase-1 RCT-264 | 0.4013032 | 0.8789263 | 0.5101573 | 0.5851339 | 0.4857754 | 0.5135371 | 1.1576368 |
| Phase-1 RCT-271 | 0.541374 | 0.7308545 | 0.6158222 | 0.6476788 | 0.713516 | 0.6503229 | 0.977524 |
| Phase-1 RCT-288 | 0.8263995 | 0.7099097 | 0.4452559 | 0.7665808 | 0.6038635 | 0.5043669 | 1.35154 |
| Phase-1 RCT-38 | 0.3242101 | 0.8159399 | 0.4129187 | 0.656195 | 0.4911781 | 0.3285534 | 1.4207782 |
| Phase-1 RCT-39 | 1.3694326 | 1.2972132 | 1.7219309 | 1.3727522 | 1.3097901 | 1.2684821 | 1.6215599 |
| Phase-1 RCT-68 | 1.5669899 | 1.0758537 | 1.2689751 | 1.235216 | 1.7443765 | 1.3422408 | 1.2381139 |
| Phase-1 RCT-33 | 0.4801861 | 0.9778237 | 0.5947639 | 0.6779819 | 0.4994276 | 0.2723433 | 1.0816144 |
| Phase-1 RCT-36 | 0.6815824 | 0.8801761 | 0.6348202 | 0.8891197 | 0.6865828 | 0.4898246 | 1.0537292 |
| 17-beta hydroxysteroid dehydrogenase, | 0.3040897 | 0.5958868 | 0.4011078 | 0.4407222 | 0.3742735 | 0.2491644 | 0.4544741 |
| type 2 | | | | | | | |
| Senescence marker protein-30 | 0.1005499 | 0.4433828 | 0.2857176 | 0.1773102 | 0.1291306 | 0.0696802 | 0.9363067 |
| Ribosomal protein S17 | 2.3944237 | 1.679237 | 1.9199592 | 1.7476137 | 1.5835245 | 1.6732677 | 1.0619301 |
| Phase-1 RCT-83 | 0.7634692 | 0.6565188 | 0.367365 | 0.421537 | 0.26431 | 0.2652865 | 0.4715055 |
| Phase-1 RCT-49 | 3.5507123 | 0.9775521 | 2.171106 | 1.2598095 | 1.7201018 | 2.7135832 | 0.9798784 |
| Phase-1 RCT-48 | 0.8570442 | 0.905743 | 0.7771382 | 0.7941555 | 0.8424852 | 0.6070359 | 0.9775963 |
| Phase-1 RCT-40 | 0.6208167 | 0.8348457 | 0.6589597 | 0.7368508 | 0.5661494 | 0.4601151 | 0.8359762 |
| Phase-1 RCT-296 | 0.1616081 | 0.3289076 | 0.1999198 | 0.3021776 | 0.2243825 | 0.1470616 | 0.8860239 |
| Phase-1 RCT-295 | 1.7048194 | 1.5465922 | 1.6781572 | 1.1982428 | 1.3465966 | 1.0343394 | 1.5046062 |
| Phase-1 RCT-291 | 0.5017113 | 0.8578391 | 0.5817197 | 0.8611236 | 0.562176 | 0.5556176 | 1.0651342 |
| Phase-1 RCT-270 | 0.4398497 | 0.980101 | 0.5247004 | 0.8135929 | 0.5145418 | 0.4535331 | 1.0843389 |
| Phase-1 RCT-241 | 1.2909565 | 1.1504853 | 1.5093951 | 1.2494702 | 1.4005688 | 1.8080631 | 0.7251883 |
| Phase-1 RCT-191 | 1.8617877 | 1.2912173 | 1.692554 | 1.2869703 | 1.8166823 | 1.2883978 | 0.9419897 |
| Phase-1 RCT-189 | 0.8520951 | 0.8551408 | 0.6569334 | 0.8185097 | 0.7883271 | 0.866738 | 1.0774376 |
| Phase-1 RCT-179 | 1.1913326 | 1.1794572 | 2.0811687 | 1.3716091 | 2.424524 | 1.9661573 | 1.1626701 |
| Phase-1 RCT-152 | 1.7552665 | 1.6590614 | 2.091083 | 1.2810138 | 1.3486449 | 1.5629497 | 1.0711147 |
| Phase-1 RCT-123 | 1.4745797 | 0.8578985 | 0.864812 | 1.1506449 | 0.863804 | 0.8971895 | 1.2070584 |
| Paraoxonase 1 | 0.4275015 | 0.6913109 | 0.40398 | 0.5798943 | 0.4473963 | 0.4704552 | 0.531076 |
| Organic anion transporter 3 | 0.8621656 | 1.0174079 | 0.9693695 | 1.1182246 | 0.7864281 | 0.5140474 | 1.3175968 |
| N-hydroxy-2-acetylaminofluorene | 0.6741163 | 0.5783891 | 0.3085378 | 0.2690423 | 0.1311424 | 0.1157278 | 0.5077717 |
| sulfotransferase (ST1C1) | | | | | | | |
| Integrin beta1 | 2.5847468 | 1.312327 | 2.2970977 | 1.3405927 | 1.9414345 | 2.4737737 | 1.7085491 |
| Insulin-like growth factor binding protein 3 | 0.4886099 | 0.5610084 | 0.4911355 | 0.5722672 | 0.3022991 | 0.3165915 | 0.6334142 |
| ID-1 | 2.306296 | 1.3842239 | 2.3010168 | 1.2042308 | 1.5516472 | 1.8843287 | 1.4431463 |
| Hepatic lipase | 1.0179372 | 0.6300731 | 0.7065453 | 0.4600447 | 0.4824779 | 0.6394408 | 0.7185957 |
| Heme oxygenase | 2.7152383 | 1.1371093 | 2.2270393 | 1.4161971 | 1.9007862 | 4.472455 | 0.9944512 |
| Equilbrative nitrobenzylthioinosine- | 0.6902381 | 0.755101 | 0.5585185 | 0.4959962 | 0.3020701 | 0.2275196 | 0.4880138 |
| sensitive nucleoside transporter | | | | | | | |
| Epidermal growth factor | 0.5529362 | 1.0283659 | 0.538563 | 0.7224632 | 1.0792471 | 0.5515144 | 0.9391982 |
| c-myc | 2.908735 | 1.0245543 | 1.7830541 | 1.298595 | 2.1622064 | 3.679885 | 1.2159842 |
| Carbonic anhydrase III | 0.0372995 | 0.2120036 | 0.1000727 | 0.1482751 | 0.1243989 | 0.078799 | 1.3109862 |
| Beta-actin | 1.9930058 | 2.1564136 | 2.9754467 | 1.625167 | 3.8749194 | 1.2742553 | 1.8392432 |
| Bax (alpha) | 1.384386 | 0.927784 | 0.9567475 | 1.1464725 | 1.1948808 | 1.2284995 | 1.5221621 |
| Alpha 1 - inhibitor III | 0.3595857 | 0.565803 | 0.2984439 | 0.6324928 | 0.3787989 | 0.43376 | 0.4302009 |
| 60S ribosomal protein L6 | 2.542786 | 2.001232 | 2.4066107 | 1.6914116 | 1.9114324 | 1.6833869 | 0.9989229 |
| Phase-1 RCT-117 | 0.5141174 | 0.7518191 | 0.4725036 | 0.6205995 | 0.4954118 | 0.601397 | 1.9381273 |
| Phase-1 RCT-102 | 0.979185 | 0.7244733 | 0.7425502 | 0.5413785 | 0.5230489 | 0.5827767 | 0.8927771 |
| 3-hydroxyisobutyrate dehydrogenase | 0.5550078 | 0.9203749 | 0.5891529 | 0.8001381 | 0.5446674 | 0.4975133 | 0.8593217 |
| ATPase inhibitor (rat mitochondrial IF1 | 0.4840354 | 0.4930837 | 0.2747284 | 0.4126689 | 0.3303557 | 0.3491139 | 0.6085519 |
| protein) | | | | | | | |
| Alpha-2-macroglobulin | 0.9328255 | 0.4453728 | 0.3616437 | 0.9099922 | 0.9736934 | 0.8452315 | 0.4820687 |
| Phase-1 RCT-137 | 0.698903 | 0.9085934 | 0.6672357 | 0.9324814 | 0.8153182 | 0.7872415 | 0.4553237 |
| Phase-1 RCT 252 | 0.7067989 | 0.9661397 | 0.8466108 | 0.8610325 | 0.8079118 | 0.6908764 | 0.6451677 |
| Phase-1 RCT-65 | 1.1251483 | 1.1012527 | 1.1493808 | 1.298894 | 1.2658174 | 0.9630997 | 1.4116539 |
| Liver fatty acid binding protein | 0.2810068 | 0.4282461 | 0.2126619 | 0.2988262 | 0.2482398 | 0.2133468 | 0.9191746 |
| Carbamyl phosphate synthetase I | 0.6927421 | 0.948971 | 0.7574574 | 0.8636691 | 0.8656945 | 0.6418412 | 0.4583764 |
| c-jun | 3.2143707 | 1.064371 | 2.2517326 | 1.7598394 | 1.7081951 | 1.7731827 | 1.7411708 |
| Transthyretin | 0.3094239 | 0.7186482 | 0.4016867 | 0.4565617 | 0.3681484 | 0.369303 | 0.7022638 |
| Aflatoxin B1 aldehyde reductase | 1.7421032 | 3.824148 | 7.065194 | 1.8123728 | 1.3896055 | 1.6392268 | 1.0137928 |
| Phase-1 RCT-15 | 1.5384948 | 1.2849332 | 1.7229642 | 1.0318732 | 1.6736032 | 1.1669209 | 1.2531292 |
| Insulin-like growth factor I | 0.4799785 | 0.5035437 | 0.3561175 | 0.3725782 | 0.2262455 | 0.2674155 | 0.600892 |
| Sodium/bile acid cotransporter | 0.3368883 | 0.9976937 | 0.8446514 | 0.7919007 | 0.6308384 | 0.4943361 | 0.3101429 |
| Organic cation transporter 3 | 2.0240536 | 1.6266541 | 1.9419141 | 1.5973607 | 1.8742828 | 1.6760845 | 0.870505 |
| Gadd153 | 2.9718585 | 1.122854 | 1.6915423 | 1.2885892 | 3.0361364 | 2.9541683 | 1.7389616 |
| Phase-1 RCT-109 | 2.4274035 | 1.5175177 | 1.8851091 | 1.60208 | 1.3969826 | 1.529359 | 1.4459845 |
| Phase-1 RCT-88 | 0.6079105 | 0.9213848 | 0.4764174 | 1.0582324 | 0.7710052 | 0.7260993 | 1.1475767 |
| Ubiquitin conjugating enzyme (RAD 6 | 1.6844543 | 1.4440753 | 1.2990458 | 1.6028544 | 1.8304756 | 1.6816338 | 0.7540053 |
| homologue) | | | | | | | |
| Glucose-regulated protein 78 | 2.46723 | 1.4468734 | 3.0839565 | 0.9292189 | 1.7311583 | 2.0894556 | 1.1500394 |
| Ribosomal protein L13A | 2.8061671 | 1.8013763 | 2.2113428 | 1.6666081 | 1.830172 | 1.5858955 | 1.6261137 |
| Monocyte chemotactic protein receptor | 2.267645 | 1.6408286 | 1.8586075 | 1.6098369 | 1.7630942 | 1.649289 | 0.8724024 |
| (CCR2) | | | | | | | |
| Fas antigen | 1.439155 | 1.3101397 | 1.1577528 | 1.3693476 | 2.1496255 | 1.1930344 | 1.658896 |
| Calpactin I heavy chain | 3.8715677 | 1.676187 | 2.189351 | 1.625362 | 2.3508852 | 2.1298687 | 0.9887139 |
| Uncoupling protein 2 | 2.4145768 | 1.3499376 | 1.6680452 | 1.8335843 | 1.7724017 | 1.6093215 | 1.5752543 |
| Phase-1 RCT-168 | 0.7336245 | 0.8596757 | 0.6214957 | 0.8598437 | 0.6615893 | 0.5881366 | 0.9644471 |
| Phase-1 RCT-154 | 2.1555882 | 1.4137173 | 1.7245204 | 1.3795067 | 1.5410916 | 1.662294 | 1.1497729 |
| Superoxide dismutase Mn | 2.9548674 | 1.518019 | 1.5104393 | 1.6049261 | 1.5720065 | 1.8577825 | 1.3448886 |
| Phase-1 RCT-214 | 0.8170111 | 0.9647705 | 0.6913356 | 0.7126156 | 0.8002648 | 0.6558819 | 0.7448598 |
| Phase-1 RCT-225 | 1.840301 | 1.6012229 | 0.4994452 | 1.803509 | 0.537821 | 1.6661315 | 0.6487556 |
| Phase-1 RCT-181 | 0.6828901 | 0.8790867 | 0.7274394 | 1.027137 | 0.9835876 | 0.8832993 | 1.0045929 |
| Nucleoside diphosphate kinase beta | 1.2519243 | 1.1696349 | 1.1105106 | 1.211217 | 1.6729065 | 1.1620513 | 0.9310227 |
| isoform | | | | | | | |
| Extracellular-signal-regulated kinase 1 | 0.0565397 | 0.3414356 | 0.2550883 | 0.1367805 | 0.140424 | 0.0794654 | 1.2907908 |
| {Ribosomal protein L6} | 2.3689332 | 1.8346372 | 2.2026622 | 1.6086929 | 1.5839182 | 1.6111103 | 1.0683833 |
| Aldehyde dehydrogenase, microsomal | 0.6933889 | 0.825416 | 0.5615354 | 0.7516701 | 0.76253 | 0.6497358 | 1.277826 |
| p53 | 1.490807 | 1.3234842 | 1.3869592 | 1.0909461 | 1.1376187 | 1.1611143 | 0.9149908 |
| Phase-1 RCT-239 | 0.65175 | 0.82122 | 0.6021984 | 0.7782507 | 0.6643653 | 0.6013878 | 1.3705628 |
| Macrophage inflammatory protein-2 alpha | 3.5830648 | 1.4085846 | 2.8965595 | 1.6478957 | 2.010488 | 2.9682014 | 1.0882816 |
| IgE binding protein | 4.9149375 | 1.675745 | 2.0075607 | 1.9184964 | 1.7290056 | 2.2816997 | 0.9575679 |
| Phase-1 RCT-205 | 1.5378289 | 1.2091863 | 1.2712562 | 1.0334775 | 1.3105564 | 1.7285874 | 0.8101422 |
| Melanoma-associated antigen ME491 | 2.0621252 | 1.4337863 | 1.3791602 | 1.1710653 | 1.2296801 | 1.4669724 | 0.986367 |
| Phase-1 RCT-242 | 1.465543 | 0.9221696 | 2.2434483 | 1.0150152 | 1.2900362 | 1.5683508 | 1.2192566 |
| Enolase alpha | 1.760203 | 1.4604919 | 1.8599303 | 1.2116262 | 1.27204 | 1.3206686 | 0.8077577 |
| Thymosin beta-10 | 2.6290216 | 1.5133142 | 1.8628675 | 1.813572 | 1.6749557 | 1.5710407 | 1.459226 |
| Cofilin | 1.7787225 | 1.2029405 | 1.2163616 | 1.2784184 | 1.2989422 | 1.1379477 | 0.802887 |
| Carbonyl reductase | 1.6894636 | 1.4748449 | 1.661004 | 1.3940912 | 1.6068825 | 1.7447444 | 0.7992331 |
| MAP kinase kinase | 1.2406976 | 1.1201105 | 1.0850257 | 0.904019 | 1.3512853 | 1.1076458 | 0.9910529 |
| Phase-1 RCT-162 | 1.6615405 | 1.3521535 | 1.6655351 | 1.4400636 | 1.4253778 | 1.3430874 | 0.9550369 |
| Phase-1 RCT-72 | 1.7905409 | 1.0706571 | 1.9403309 | 1.3332051 | 1.3714188 | 1.3946064 | 1.135691 |
| Ribosomal protein S8 | 2.575565 | 1.8624856 | 2.1455233 | 1.7219293 | 1.5348426 | 1.5116693 | 0.9564226 |
| Proliferating cell nuclear antigen gene | 1.2739083 | 0.8845056 | 1.2145532 | 1.0146555 | 1.0157743 | 1.6634737 | 0.9228275 |
| Phase-1 RCT-192 | 1.7672387 | 1.3316823 | 1.7472245 | 1.1747884 | 1.4558609 | 1.1726967 | 1.0165868 |
| IkB-a | 1.7262594 | 1.3872033 | 1.8576231 | 1.1632226 | 1.1811992 | 1.3579197 | 1.0526273 |
| Endogenous retroviral sequence, 5′ and | 1.1083918 | 1.911932 | 0.8517811 | 1.9804286 | 0.8468149 | 1.4089309 | 0.7794915 |
| 3′ LTR | | | | | | | |
| Phase-1 RCT-37 | 1.8286437 | 1.5163486 | 1.8210925 | 1.3302857 | 1.441814 | 1.3576527 | 1.1869764 |
| Matrix metalloproteinase-1 | 1.9700292 | 1.6615969 | 2.3332312 | 1.0998316 | 1.1650577 | 1.2953582 | 1.864509 |
| Cyclin G | 2.4211576 | 1.1573857 | 1.2865088 | 1.7635794 | 2.2096837 | 2.8959618 | 2.0284479 |
| DNA polymerase beta | 1.2825508 | 1.2698765 | 1.2297217 | 1.0803038 | 1.1799638 | 1.0104771 | 0.7000579 |
| Phase-1 RCT-127 | 1.4337729 | 0.8181927 | 1.307704 | 0.992536 | 0.7233632 | 1.7249565 | 1.8457665 |
| Multidrug resistant protein-2 | 6.1735992 | 1.905452 | 4.252751 | 2.902582 | 2.4494357 | 2.5084052 | 1.5861276 |
| Ribosomal protein S9 | 1.8109064 | 1.1725705 | 1.1839602 | 1.3214 | 1.6653782 | 1.5268471 | 0.6788492 |
| Phase-1 RCT-12 | 1.9253509 | 1.1359744 | 1.2756582 | 1.0106765 | 1.5688204 | 1.318987 | 0.8789671 |
| Beta-tubulin, class I | 2.8506553 | 1.182604 | 1.6753527 | 1.0757418 | 2.314277 | 1.4674935 | 0.7992553 |
| Argininosuccinate lyase | 1.9414912 | 1.4516863 | 1.8944308 | 1.1376382 | 1.861319 | 1.3540685 | 1.1580272 |
| Elongation factor-1 alpha | 2.102706 | 1.4968712 | 1.7153046 | 1.3815624 | 1.3516675 | 1.2004886 | 1.3969063 |
| Phase-1 RCT-55 | 0.9118204 | 1.4107014 | 2.630658 | 1.3448384 | 2.0394783 | 1.9787757 | 1.0319189 |
| Macrophage inflammatory protein-1 alpha | 1.7506641 | 1.4494371 | 1.6503798 | 1.4451402 | 1.8146522 | 1.827477 | 1.0340112 |
| c-H-ras | 1.3969246 | 1.0318757 | 0.9768938 | 1.2015506 | 1.0392032 | 1.0117741 | 0.9752517 |
| Multidrug resistant protein-1 | 10.695147 | 1.876597 | 4.234022 | 4.0481467 | 3.405581 | 3.0688853 | 1.7220082 |
| T-cell cyclophilin | 1.3725969 | 1.3619719 | 1.3657271 | 1.11287 | 1.1340784 | 0.969251 | 0.7418268 |
| Phase-1 RCT-185 | 0.3866216 | 0.6933857 | 0.5684025 | 0.6394927 | 0.4921462 | 0.519972 | 0.74057 |
| 25-DX | 0.9731351 | 1.5323958 | 1.5084121 | 1.2160286 | 1.0874338 | 1.2160532 | 1.2341386 |
| Ref-1 | 1.3498952 | 1.3052413 | 1.5201982 | 1.2438059 | 1.7135047 | 1.8923337 | 0.9318656 |
| Apolipoprotein CIII | 0.4918512 | 0.9200652 | 0.7942825 | 0.7777448 | 0.7203525 | 0.9100906 | 0.8156238 |
| NADPH quinone oxidoreductase-1 (DT- | 1.490646 | 1.4868166 | 1.5710475 | 1.6829526 | 1.905352 | 2.524841 | 0.5802039 |
| diaphorase) | | | | | | | |
| Pyruvate kinase, muscle | 3.186226 | 1.3090701 | 2.158049 | 2.101238 | 2.3172796 | 2.2965617 | 1.395751 |
| | CHLOR | CHLOR | CHLOR | CHLOR | CIS | CIS | CIS |
| Compound-Dose (2) | 8 | 8 | 30 | 30 | 2.5 | 2.5 | 10 |
| Animal Number (3) | 45 | 46 | 55 | 56 | 324 | 326 | 334 |
| Liver Toxicity Necrosis Classification (4) | no | no | no | no | no | no | no |
| Gene Name (5) | | | | | | | |
| Phase-1 RCT-78 | 0.8607835 | 0.9622628 | 1.0011305 | 1.2277693 | 0.9451106 | 1.076718 | 0.8073915 |
| Gamma-actin, cytoplasmic | 1.0337392 | 1.1692146 | 1.4031113 | 1.029407 | 0.6282655 | 0.8338799 | 0.8272463 |
| Matrin F/G | 1.9299235 | 1.5319067 | 1.3676157 | 1.3103834 | 0.9483665 | 0.9101452 | 1.2457743 |
| Zinc finger protein | 0.9464596 | 0.8920125 | 0.9239103 | 0.905196 | 1.3234811 | 1.0800295 | 0.9822273 |
| Cathepsin L, sequence 2 | 0.974179 | 0.9772522 | 0.9147419 | 1.3529702 | 0.9270976 | 1.2141223 | 1.4807463 |
| Gadd45 | 0.9012383 | 0.7240961 | 0.9829873 | 1.5217386 | 1.1479658 | 1.2180996 | 1.319409 |
| Phase-1 RCT-144 | 0.9438536 | 0.9757195 | 0.978453 | 0.9817358 | 0.8553296 | 0.8575144 | 0.9556294 |
| Phase-1 RCT-145 | 1.0466523 | 1.2440423 | 1.1851947 | 1.133658 | 1.0370014 | 1.0323118 | 0.9932321 |
| Phase-1 RCT-50 | 1.092173 | 1.227109 | 1.1130536 | 1.4127111 | 1.238596 | 0.8719963 | 1.3135983 |
| Phase-1 RCT-92 | 0.8432889 | 1.1401483 | 0.8841939 | 1.0948187 | 0.7579902 | 0.9846339 | 0.7355877 |
| Phase-1 RCT-89 | 1.1753924 | 0.9550012 | 0.7726266 | 0.6823449 | 0.5829307 | 0.9435955 | 0.5203279 |
| 14-3-3 zeta | 0.9678749 | 1.5040133 | 1.5475848 | 1.3681266 | 0.9423689 | 0.907474 | 0.8497372 |
| Dynamin-1 (D100) | 1.0765752 | 0.9836102 | 0.8106828 | 0.7902838 | 1.1144723 | 0.8485215 | 0.8961323 |
| Insulin-like growth factor binding protein 1 | 1.6031494 | 1.5778029 | 1.2130103 | 2.0580611 | 0.6197209 | 0.9836901 | 0.7644331 |
| L-gulono-gamma-lactone oxidase | 1.6266693 | 1.0194757 | 1.0674201 | 1.3262844 | 0.6216647 | 0.5610655 | 0.3933854 |
| Ornithine decarboxylase | 1.7626339 | 3.480216 | 3.6108851 | 2.2009513 | 1.3402053 | 0.9507311 | 1.3365344 |
| PAR interacting protein | 0.9626226 | 1.0083088 | 1.0596166 | 1.0567555 | 0.9231157 | 1.023355 | 0.9423729 |
| Phase-1 RCT-128 | 1.1015064 | 1.0157777 | 0.9033173 | 0.8764695 | 0.5434216 | 0.7163746 | 0.6201788 |
| Phase-1 RCT-180 | 0.9469357 | 0.9715555 | 1.038763 | 0.9459478 | 1.6229701 | 1.5380939 | 1.4608333 |
| Phase-1 RCT-182 | 0.8853326 | 0.8484109 | 0.8879759 | 0.8455561 | 0.770283 | 1.2042272 | 0.7537985 |
| Phase-1 RCT-207 | 0.9970184 | 1.117147 | 1.1248664 | 1.0781369 | 1.2034016 | 1.0987779 | 1.7326708 |
| Phase-1 RCT-213 | 1.0487964 | 1.1235648 | 1.2339238 | 1.1430113 | 1.2300916 | 1.154293 | 1.1003022 |
| Phase-1 RCT-256 | 1.9600946 | 1.5241872 | 1.4906304 | 1.5264934 | 0.5318754 | 0.7647484 | 0.6253275 |
| Phase-1 RCT-258 | 0.985078 | 0.9363327 | 1.1128812 | 0.9594027 | 1.1790006 | 0.9807834 | 0.9606846 |
| Phase-1 RCT-264 | 1.221213 | 1.9757322 | 1.5182499 | 1.3116298 | 0.5811398 | 0.743714 | 0.559315 |
| Phase-1 RCT-271 | 1.153234 | 0.9167817 | 0.7956316 | 0.8888957 | 0.9208229 | 0.7897346 | 0.6425568 |
| Phase-1 RCT-288 | 1.3519984 | 1.293204 | 1.2750105 | 1.0913733 | 0.7306607 | 0.8442872 | 0.5797194 |
| Phase-1 RCT-38 | 1.7188926 | 1.3251982 | 1.2376769 | 1.240316 | 0.4940798 | 0.6773579 | 0.6243151 |
| Phase-1 RCT-39 | 1.4115503 | 1.349628 | 1.1377786 | 1.0491626 | 0.8174416 | 0.887948 | 1.0494285 |
| Phase-1 RCT-68 | 1.0250171 | 1.1526634 | 1.362027 | 1.2501867 | 1.1098714 | 0.9460027 | 1.285993 |
| Phase-1 RCT-33 | 1.0740836 | 1.060955 | 0.9927216 | 1.0857751 | 0.8753851 | 0.70147 | 0.5556629 |
| Phase-1 RCT-36 | 1.0967163 | 1.0812649 | 0.9569398 | 1.1193799 | 1.18202 | 0.8520767 | 0.8738113 |
| 17-beta hydroxysteroid dehydrogenase, | 0.4987307 | 0.3944599 | 0.3572585 | 0.440654 | 0.7231686 | 0.9691052 | 0.7046318 |
| type 2 | | | | | | | |
| Senescence marker protein-30 | 0.776018 | 0.7185239 | 0.5580232 | 0.3917894 | 0.570138 | 1.2732934 | 0.3511908 |
| Ribosomal protein S17 | 1.0674578 | 1.1445694 | 1.4758997 | 1.2568531 | 0.7611532 | 1.3734509 | 1.1439061 |
| Phase-1 RCT-83 | 0.5791216 | 0.5026998 | 0.3634665 | 0.3217674 | 1.112734 | 0.9115802 | 0.6323901 |
| Phase-1 RCT-49 | 1.041376 | 0.9428289 | 0.9833307 | 1.0567831 | 1.0938528 | 0.7877361 | 1.1182084 |
| Phase-1 RCT-48 | 0.7984275 | 1.2208415 | 0.9456223 | 1.0503836 | 0.7335326 | 0.7065628 | 0.5865915 |
| Phase-1 RCT-40 | 1.0648195 | 0.966093 | 0.9420123 | 0.64845 | 0.7344165 | 1.2431074 | 0.5244213 |
| Phase-1 RCT-296 | 1.3135695 | 1.0867846 | 1.3487991 | 1.2111536 | 1.0185341 | 0.8289453 | 0.2318363 |
| Phase-1 RCT-295 | 1.2954656 | 1.3453301 | 1.2795527 | 1.2840881 | 0.7899448 | 0.8135337 | 1.0130997 |
| Phase-1 RCT-291 | 1.1661826 | 1.0382669 | 0.892646 | 0.9805633 | 0.9382803 | 1.2458768 | 0.9352306 |
| Phase-1 RCT-270 | 1.2404761 | 1.0791998 | 0.8868678 | 0.9352942 | 0.7815947 | 1.2509441 | 0.7395225 |
| Phase-1 RCT-241 | 0.7325096 | 0.7304468 | 0.8057044 | 0.6494042 | 1.5475765 | 1.2616662 | 1.1250352 |
| Phase-1 RCT-191 | 0.7231351 | 0.7399314 | 0.6422824 | 0.6224721 | 2.3776848 | 1.0801154 | 2.2285156 |
| Phase-1 RCT-189 | 1.603178 | 1.5266793 | 1.2138711 | 1.1819234 | 0.782797 | 1.079618 | 0.800868 |
| Phase-1 RCT-179 | 1.1315386 | 1.0255935 | 0.9077433 | 0.8175134 | 0.8838185 | 0.9251663 | 0.7389568 |
| Phase-1 RCT-152 | 0.9413447 | 0.7373906 | 0.8022502 | 0.8201085 | 0.7496445 | 1.1664348 | 1.0833834 |
| Phase-1 RCT-123 | 0.9417501 | 0.9382809 | 0.9047302 | 0.9276012 | 1.2527659 | 0.8841026 | 1.366702 |
| Paraoxonase 1 | 0.4745067 | 0.5320101 | 0.4815271 | 0.5021851 | 0.5326357 | 0.9353516 | 0.4450429 |
| Organic anion transporter 3 | 1.4536006 | 0.9768471 | 0.6902948 | 1.0882787 | 0.5749831 | 0.6959701 | 0.6531905 |
| N-hydroxy-2-acetylaminofluorene | 0.5331641 | 0.4778536 | 0.4031999 | 0.4145576 | 0.4318642 | 0.796662 | 0.2144646 |
| sulfotransferase (ST1C1) | | | | | | | |
| Integrin beta1 | 1.4780182 | 1.4246706 | 1.6488527 | 2.685007 | 0.6845577 | 0.9422278 | 0.9165761 |
| Insulin-like growth factor binding protein 3 | 0.9652056 | 0.7630577 | 0.5337548 | 0.5990519 | 0.8430029 | 0.7961096 | 0.6845434 |
| ID-1 | 1.2933875 | 1.1104575 | 1.0960327 | 1.2530203 | 1.1930537 | 1.0442795 | 1.0556043 |
| Hepatic lipase | 0.8227764 | 0.5628368 | 0.4684192 | 0.6672351 | 0.4740529 | 0.7814818 | 0.2799614 |
| Heme oxygenase | 1.0782809 | 0.817642 | 0.7261281 | 0.7116813 | 0.9934096 | 0.8510598 | 1.0008491 |
| Equilbrative nitrobenzylthioinosine- | 0.6103079 | 0.5556062 | 0.4252173 | 0.3997742 | 0.6266845 | 0.9848797 | 0.7262129 |
| sensitive nucleoside transporter | | | | | | | |
| Epidermal growth factor | 0.7337751 | 0.8450263 | 1.375353 | 0.9962146 | 0.7929972 | 1.186321 | 1.080053 |
| c-myc | 1.0019932 | 1.3436288 | 1.1122122 | 1.1182342 | 1.6878546 | 1.7297822 | 1.6824652 |
| Carbonic anhydrase III | 1.1384124 | 0.8681812 | 0.5592448 | 0.3262349 | 0.2088967 | 0.4927986 | 0.127679 |
| Beta-actin | 1.9995865 | 2.2815988 | 3.8257785 | 2.5394576 | 0.6554335 | 0.8867914 | 0.5439541 |
| Bax (alpha) | 1.1597836 | 1.525885 | 1.1983931 | 0.9819619 | 1.366779 | 1.0283289 | 1.1118253 |
| Alpha 1 - inhibitor III | 0.8532909 | 0.6721681 | 0.5052064 | 0.5393853 | 0.5836702 | 0.9722624 | 0.4164851 |
| 60S ribosomal protein L6 | 0.9725 | 0.9324464 | 0.8363535 | 1.0505217 | 1.2775595 | 1.0421566 | 0.9732808 |
| Phase-1 RCT-117 | 1.670123 | 1.3210906 | 1.2966324 | 0.961409 | 0.7629294 | 1.032305 | 0.8138116 |
| Phase-1 RCT-102 | 0.8443456 | 0.8632546 | 0.5806189 | 0.629146 | 0.8477321 | 0.528174 | 0.2462262 |
| 3-hydroxyisobutyrate dehydrogenase | 0.9054585 | 1.1365039 | 1.2210319 | 0.6879672 | 0.7243433 | 1.080903 | 0.7081417 |
| ATPase inhibitor (rat mitochondrial IF1 | 0.6040159 | 0.5048392 | 0.5142837 | 0.5303346 | 0.4821537 | 0.8605969 | 0.459627 |
| protein) | | | | | | | |
| Alpha-2-macroglobulin | 1.0187674 | 0.9294365 | 1.1914253 | 0.7748953 | 0.7339491 | 1.0894071 | 0.6363683 |
| Phase-1 RCT-137 | 0.5268652 | 0.476658 | 0.4004778 | 0.4707787 | 0.7474079 | 1.0283815 | 0.6737796 |
| Phase-1 RCT 252 | 1.2239488 | 1.0584385 | 0.8971111 | 1.397953 | 0.5530085 | 0.7793449 | 1.1168917 |
| Phase-1 RCT-65 | 1.4356123 | 1.1765527 | 1.1819371 | 1.115687 | 2.0979779 | 0.9428421 | 1.7487537 |
| Liver fatty acid binding protein | 0.9237759 | 0.5292841 | 0.3743474 | 0.4119408 | 0.8953968 | 0.9201109 | 0.2922781 |
| Carbamyl phosphate synthetase I | 1.5058508 | 1.1540596 | 0.6273214 | 0.9016648 | 0.5247682 | 0.7749379 | 1.1022915 |
| c-jun | 1.3544605 | 1.6517537 | 1.3955567 | 1.7649726 | 2.2173843 | 0.87656 | 1.9329888 |
| Transthyretin | 0.3103809 | 0.3831937 | 0.2998863 | 0.3111739 | 0.5601787 | 0.6198754 | 0.5614568 |
| Aflatoxin B1 aldehyde reductase | 0.5991697 | 0.5881491 | 0.376633 | 0.4445136 | 0.356518 | 0.8018236 | 0.6488218 |
| Phase-1 RCT-15 | 1.3914596 | 2.0069695 | 1.919852 | 1.6354334 | 1.5021759 | 1.0076462 | 1.5440177 |
| Insulin-like growth factor I | 0.8811973 | 0.658025 | 0.4975352 | 0.6370642 | 0.7167889 | 0.772683 | 0.4764601 |
| Sodium/bile acid cotransporter | 0.5668975 | 0.3329743 | 0.3560306 | 0.366156 | 0.7885116 | 0.985213 | 0.4704706 |
| Organic cation transporter 3 | 0.8592352 | 0.8740543 | 0.8580075 | 1.037833 | 1.4260577 | 0.9630614 | 0.9628981 |
| Gadd153 | 1.0445358 | 1.2607881 | 1.3025993 | 1.105625 | 0.8940916 | 0.9411652 | 1.4060813 |
| Phase-1 RCT-109 | 1.1852068 | 1.0831156 | 1.1038141 | 1.2159032 | 0.6416536 | 0.7212179 | 0.8799327 |
| Phase-1 RCT-88 | 0.9766096 | 1.3402203 | 1.1951985 | 1.3873786 | 1.2513545 | 1.0347621 | 1.1227387 |
| Ubiquitin conjugating enzyme (RAD 6 | 0.7793418 | 0.7097029 | 0.5934899 | 0.7164316 | 0.8150982 | 0.9420985 | 0.9209402 |
| homologue) | | | | | | | |
| Glucose-regulated protein 78 | 1.0472488 | 1.0706767 | 4.2098007 | 2.7762258 | 0.2897015 | 0.8058562 | 0.3652417 |
| Ribosomal protein L13A | 1.5332355 | 1.3256055 | 1.6739731 | 1.8008224 | 0.6684468 | 0.6651767 | 0.7975397 |
| Monocyte chemotactic protein receptor | 0.8373629 | 0.8333499 | 0.8154045 | 1.0169382 | 1.7226957 | 1.116666 | 1.2830446 |
| (CCR2) | | | | | | | |
| Fas antigen | 1.4236131 | 1.8036429 | 3.0106614 | 2.3859372 | 1.1788747 | 1.3331988 | 0.72531 |
| Calpactin I heavy chain | 1.0540596 | 1.1944932 | 0.9957113 | 0.8625778 | 1.2444483 | 1.0537971 | 1.2428876 |
| Uncoupling protein 2 | 1.3532453 | 1.2117263 | 1.2734271 | 1.5674418 | 1.2918465 | 1.0248822 | 1.0589432 |
| Phase-1 RCT-168 | 1.1974081 | 0.9763854 | 1.0983257 | 1.1990459 | 0.7025182 | 0.8263165 | 0.689428 |
| Phase-1 RCT-154 | 0.9865828 | 1.2215265 | 1.1662236 | 1.1053346 | 0.9960799 | 1.0005022 | 2.0589685 |
| Superoxide dismutase Mn | 1.1202965 | 1.4254324 | 1.4426531 | 1.6017439 | 1.094141 | 0.8849636 | 1.2139889 |
| Phase-1 RCT-214 | 0.8866593 | 0.9831879 | 1.0666806 | 0.8784125 | 0.8843097 | 0.9111136 | 0.4349275 |
| Phase-1 RCT-225 | 1.1654569 | 1.148871 | 0.8907595 | 0.4840235 | 1.0237489 | 0.7615722 | 1.1903867 |
| Phase-1 RCT-181 | 1.0409751 | 1.216856 | 1.2604249 | 1.2534215 | 1.031918 | 0.957369 | 0.9145681 |
| Nucleoside diphosphate kinase beta | 0.9181216 | 0.9987968 | 1.3606321 | 1.1420627 | 1.1557537 | 1.6733311 | 1.252927 |
| isoform | | | | | | | |
| Extracellular-signal-regulated kinase 1 | 1.1054016 | 0.8437554 | 0.5599762 | 0.3071311 | 1.1886556 | 0.7304365 | 1.1068233 |
| {Ribosomal protein L6} | 1.3100564 | 1.1624311 | 1.1506336 | 1.5571465 | 0.9207551 | 1.0123564 | 1.2395573 |
| Aldehyde dehydrogenase, microsomal | 1.0955907 | 1.0034623 | 1.1065545 | 1.0775802 | 1.0665894 | 1.150418 | 0.9708295 |
| p53 | 1.0283021 | 0.988948 | 1.0234778 | 1.0392927 | 0.8940103 | 0.9495751 | 0.8076869 |
| Phase-1 RCT-239 | 1.5416204 | 1.2209153 | 1.0724903 | 1.3306426 | 1.6431084 | 1.0658231 | 1.8452631 |
| Macrophage inflammatory protein-2 alpha | 0.7840559 | 1.0066804 | 1.5919212 | 1.0074661 | 1.5734266 | 1.2599277 | 4.758492 |
| IgE binding protein | 1.1243645 | 1.2497979 | 1.2329196 | 0.977197 | 1.2092645 | 0.8146161 | 1.2889707 |
| Phase-1 RCT-205 | 0.9596918 | 0.9565604 | 0.9663383 | 0.9518551 | 1.1121995 | 1.1161385 | 1.0723933 |
| Melanoma-associated antigen ME491 | 1.0736835 | 0.9857694 | 1.0015023 | 0.9784083 | 0.9698131 | 1.013202 | 1.2892904 |
| Phase-1 RCT-242 | 1.1172447 | 1.2097231 | 1.1518385 | 1.0520346 | 1.2966644 | 1.0386639 | 1.6458608 |
| Enolase alpha | 1.5646904 | 1.15621 | 1.6185347 | 1.8946108 | 0.5593312 | 0.9973083 | 0.9922746 |
| Thymosin beta-10 | 1.2739258 | 1.0923687 | 1.1484659 | 1.5247775 | 0.7746214 | 0.9903091 | 1.0044553 |
| Cofilin | 0.8435684 | 0.9664053 | 0.8996427 | 0.7662689 | 0.9727799 | 1.3761318 | 1.1655151 |
| Carbonyl reductase | 0.8595761 | 0.8156682 | 0.7642246 | 0.9103994 | 1.292442 | 1.2107755 | 1.2957275 |
| MAP kinase kinase | 0.7745083 | 0.9344583 | 1.3151636 | 1.0814239 | 0.6162756 | 0.8903453 | 0.8004822 |
| Phase-1 RCT-162 | 0.8462222 | 0.8259528 | 0.7469191 | 0.8314185 | 0.9932673 | 1.1413177 | 1.1681024 |
| Phase-1 RCT-72 | 1.0744566 | 1.0393142 | 0.9990431 | 0.9493681 | 0.8320233 | 0.9461111 | 1.1017557 |
| Ribosomal protein S8 | 0.7758628 | 0.7608836 | 0.7177711 | 0.7847602 | 0.7871742 | 1.2352318 | 1.1016206 |
| Proliferating cell nuclear antigen gene | 0.7554146 | 0.931667 | 0.8009754 | 0.9326549 | 0.9411848 | 1.0303925 | 1.1966109 |
| Phase-1 RCT-192 | 1.1344378 | 1.0388281 | 1.2017347 | 1.2925192 | 0.9820766 | 1.3029827 | 1.1504003 |
| IkB-a | 1.5075887 | 1.1326232 | 1.5538621 | 2.0592947 | 0.5494151 | 0.7223972 | 0.8647373 |
| Endogenous retroviral sequence, 5′ and | 1.4570918 | 1.1854516 | 1.2431531 | 1.539076 | 0.5092347 | 0.5646141 | 0.8274848 |
| 3′ LTR | | | | | | | |
| Phase-1 RCT-37 | 1.2574722 | 1.1988237 | 1.2206571 | 1.538637 | 1.1340243 | 1.0140313 | 1.1972648 |
| Matrix metalloproteinase-1 | 1.7199522 | 1.5294427 | 1.6043587 | 2.1731188 | 0.9782881 | 1.2348429 | 0.9856398 |
| Cyclin G | 0.8354939 | 1.0905935 | 1.2311877 | 0.9744952 | 1.3104947 | 2.2622194 | 4.177021 |
| DNA polymerase beta | 0.5424109 | 0.5217003 | 0.4231622 | 0.613861 | 0.9630513 | 1.0333036 | 1.0247248 |
| Phase-1 RCT-127 | 1.6520334 | 1.3075392 | 1.2172813 | 0.9211795 | 1.1035542 | 0.9831564 | 1.158282 |
| Multidrug resistant protein-2 | 1.1946652 | 1.2312013 | 1.109738 | 1.7793016 | 0.9401709 | 1.639345 | 2.9246273 |
| Ribosomal protein S9 | 0.6371657 | 0.6926009 | 0.6014586 | 0.7177029 | 0.9146004 | 1.5035032 | 0.9522787 |
| Phase-1 RCT-12 | 1.0238414 | 1.0179986 | 1.0760834 | 0.9468527 | 1.2084297 | 0.7116929 | 1.0336585 |
| Beta-tubulin, class I | 0.8457782 | 0.8946962 | 0.8345172 | 0.8536087 | 1.3677133 | 0.5222045 | 0.8286044 |
| Argininosuccinate lyase | 0.808817 | 1.1280862 | 0.8104566 | 0.7526506 | 1.103141 | 0.5909745 | 2.0967388 |
| Elongation factor-1 alpha | 1.7177944 | 1.2449312 | 1.1452734 | 1.3346578 | 0.624918 | 1.4390482 | 0.8224856 |
| Phase-1 RCT-55 | 1.0702797 | 1.196943 | 1.5539397 | 1.0692372 | 0.7408477 | 0.7992042 | 0.9623083 |
| Macrophage inflammatory protein-1 alpha | 0.9835348 | 0.9776784 | 0.7989588 | 0.9818819 | 1.5730629 | 1.0326964 | 1.7944839 |
| c-H-ras | 1.3131206 | 1.1269218 | 1.3974054 | 1.3004295 | 1.2268939 | 0.8330191 | 1.1560876 |
| Multidrug resistant protein-1 | 1.6474925 | 1.9830111 | 1.5504704 | 1.9396741 | 1.0010531 | 1.8931203 | 5.2112646 |
| T-cell cyclophilin | 0.570146 | 0.5545211 | 0.4526081 | 0.6225194 | 0.7667258 | 1.3311896 | 0.9044762 |
| Phase-1 RCT-185 | 0.9012877 | 0.7429908 | 0.6512678 | 0.6604155 | 0.5877339 | 0.8644245 | 0.5725222 |
| 25-DX | 1.821772 | 1.3431692 | 2.120996 | 1.9546307 | 0.537659 | 0.8948053 | 0.6324779 |
| Ref-1 | 1.2621802 | 1.1289668 | 1.1458676 | 1.1772068 | 0.8989914 | 0.8429269 | 1.127526 |
| Apolipoprotein CIII | 0.685941 | 0.7565933 | 0.8916003 | 0.7841036 | 1.0393406 | 1.1187668 | 1.0711987 |
| NADPH quinone oxidoreductase-1 (DT- | 1.0446845 | 0.7691945 | 0.8555439 | 1.1638571 | 0.9874752 | 1.2574757 | 1.9799837 |
| diaphorase) | | | | | | | |
| Pyruvate kinase, muscle | 1.0498942 | 1.0295482 | 1.112782 | 1.3712785 | 1.8185265 | 0.7661569 | 1.0308839 |
| | CIS | CIS | CLO | CLO | CLO | CLO | CLO |
| Compound-Dose (2) | 10 | 10 | 75 | 75 | 75 | 250 | 250 |
| Animal Number (3) | 335 | 336 | 324 | 325 | 326 | 1844 | 1845 |
| Liver Toxicity Necrosis Classification (4) | no | no | no | no | no | no | no |
| Gene Name (5) | | | | | | | |
| Phase-1 RCT-78 | 0.8046168 | 0.9871655 | 1.0238378 | 1.0873106 | 1.1120224 | 1.037546 | 1.0312718 |
| Gamma-actin, cytoplasmic | 0.5751056 | 0.6795235 | 1.0778693 | 0.7838244 | 0.8632041 | 0.7769618 | 0.7060872 |
| Matrin F/G | 1.2083122 | 0.9624211 | 1.221967 | 1.0167148 | 1.1361852 | 1.4009918 | 1.1008941 |
| Zinc finger protein | 1.1447985 | 1.0847522 | 0.9179699 | 0.9378578 | 0.8106546 | 1.0869216 | 1.0245874 |
| Cathepsin L, sequence 2 | 1.6676673 | 1.4350458 | 1.0773492 | 1.0117935 | 0.9565347 | 1.0829068 | 0.9754005 |
| Gadd45 | 1.3900796 | 1.0893645 | 1.1656715 | 0.7616876 | 1.1293821 | 1.0778009 | 0.9973549 |
| Phase-1 RCT-144 | 0.9701576 | 0.8475664 | 0.9703862 | 0.8121525 | 0.9202232 | 0.931891 | 0.9180039 |
| Phase-1 RCT-145 | 1.0810103 | 0.9703174 | 0.971588 | 1.025661 | 0.9542384 | 1.0116105 | 1.0194573 |
| Phase-1 RCT-50 | 1.037604 | 0.8177524 | 1.1114589 | 1.0868558 | 1.0424821 | 1.2225633 | 0.9798389 |
| Phase-1 RCT-92 | 0.708987 | 1.3046607 | 0.9014301 | 0.7500759 | 0.743497 | 0.6614122 | 0.7875341 |
| Phase-1 RCT-89 | 0.6633486 | 0.9986178 | 0.8670437 | 0.905162 | 0.8961787 | 1.0506935 | 1.1200917 |
| 14-3-3 zeta | 0.9103854 | 1.1311103 | 1.2143873 | 1.220523 | 1.170079 | 1.1488966 | 1.192529 |
| Dynamin-1 (D100) | 0.8578991 | 1.1988286 | 1.0052273 | 1.0230565 | 0.8908589 | 0.9982128 | 1.0824605 |
| Insulin-like growth factor binding protein 1 | 1.0143567 | 1.32067 | 1.4721029 | 1.6323174 | 1.504116 | 1.680814 | 1.5471383 |
| L-gulono-gamma-lactone oxidase | 0.3640076 | 0.5067522 | 0.8005467 | 0.7057471 | 0.5025994 | 0.6946214 | 0.775735 |
| Ornithine decarboxylase | 1.0972922 | 0.7047622 | 1.1665722 | 0.8244281 | 0.9825374 | 0.9671012 | 0.9300174 |
| PAR interacting protein | 0.9236925 | 0.8311345 | 1.049854 | 1.0367074 | 0.9792762 | 1.010814 | 0.9554628 |
| Phase-1 RCT-128 | 0.5982262 | 1.6249751 | 0.9893356 | 1.0063609 | 0.8046587 | 0.940525 | 1.2254317 |
| Phase-1 RCT-180 | 1.2252749 | 1.0601547 | 1.0851306 | 0.9510201 | 1.0504035 | 0.9011508 | 1.0153394 |
| Phase-1 RCT-182 | 0.7754691 | 1.0924189 | 0.7678316 | 0.7665625 | 0.7374777 | 1.0332078 | 1.0206137 |
| Phase-1 RCT-207 | 1.7344229 | 1.5880327 | 1.0974342 | 1.211136 | 1.0325438 | 1.2120092 | 1.1693616 |
| Phase-1 RCT-213 | 1.1370479 | 0.8928941 | 0.9979489 | 1.1325656 | 0.9982649 | 1.0386674 | 0.9940351 |
| Phase-1 RCT-256 | 0.5855819 | 1.1167219 | 1.152052 | 0.9915163 | 0.9034577 | 0.8178946 | 1.0530919 |
| Phase-1 RCT-258 | 1.0502334 | 0.7975624 | 0.992701 | 1.0548091 | 0.9146017 | 1.0935695 | 1.0810217 |
| Phase-1 RCT-264 | 0.6364979 | 1.3715794 | 0.6718445 | 0.7697249 | 0.636275 | 0.563508 | 0.8388788 |
| Phase-1 RCT-271 | 0.4463132 | 1.1051266 | 0.8834405 | 0.8655503 | 0.6716755 | 0.7088648 | 0.9824195 |
| Phase-1 RCT-288 | 0.5864734 | 0.8905542 | 0.9812582 | 0.9747835 | 0.8670272 | 1.0427788 | 1.1108547 |
| Phase-1 RCT-38 | 0.6190891 | 1.1063186 | 1.0868759 | 1.0166274 | 0.8173037 | 0.7791298 | 0.9831153 |
| Phase-1 RCT-39 | 0.9999999 | 0.7731459 | 0.9408482 | 1.0248312 | 1.1853719 | 1.0179993 | 1.0109497 |
| Phase-1 RCT-68 | 1.0019991 | 1.1050183 | 1.0381862 | 1.0394188 | 1.0161443 | 1.0323689 | 1.0321838 |
| Phase-1 RCT-33 | 0.5999613 | 0.6128426 | 1.0091028 | 0.8823876 | 0.86417 | 0.9027408 | 1.1406156 |
| Phase-1 RCT-36 | 0.8501622 | 0.7313553 | 1.0184898 | 0.9492431 | 0.8762108 | 0.925804 | 1.0708504 |
| 17-beta hydroxysteroid dehydrogenase, | 0.6496035 | 0.1475818 | 0.7709408 | 0.9076842 | 0.8512438 | 0.6846164 | 1.8661739 |
| type 2 | | | | | | | |
| Senescence marker protein-30 | 0.42047 | 0.9719585 | 0.9864362 | 1.0504204 | 0.8480418 | 0.9684312 | 0.9954882 |
| Ribosomal protein S17 | 1.0878431 | 1.9202831 | 0.9832848 | 1.1760634 | 1.0018028 | 0.8554587 | 0.9687598 |
| Phase-1 RCT-83 | 0.6946861 | 0.8835927 | 0.8277054 | 1.0008863 | 0.9380672 | 0.9266082 | 0.8997337 |
| Phase-1 RCT-49 | 0.9792716 | 0.8867833 | 0.9397801 | 0.9519593 | 1.06024 | 1.0177245 | 1.0266343 |
| Phase-1 RCT-48 | 0.5791497 | 1.3059179 | 0.9763258 | 1.0195045 | 0.9937492 | 0.7691087 | 0.8511204 |
| Phase-1 RCT-40 | 0.7598361 | 1.1957912 | 0.8566524 | 0.8540801 | 0.8933591 | 0.7665758 | 0.9859136 |
| Phase-1 RCT-296 | 0.2450759 | 0.3108751 | 0.8845093 | 0.6070795 | 0.5058811 | 0.5518755 | 0.7134052 |
| Phase-1 RCT-295 | 0.921299 | 1.143048 | 1.137225 | 1.1930573 | 1.0916301 | 1.013991 | 1.0759624 |
| Phase-1 RCT-291 | 0.9680121 | 1.1965643 | 0.8452637 | 0.7624245 | 0.8178515 | 0.8285808 | 0.843803 |
| Phase-1 RCT-270 | 0.6294899 | 0.8483383 | 0.8774688 | 0.8479526 | 0.7589024 | 0.6459318 | 0.9349173 |
| Phase-1 RCT-241 | 1.3712897 | 0.922298 | 1.0128397 | 1.0734928 | 1.0972037 | 1.0168953 | 0.9553103 |
| Phase-1 RCT-191 | 2.0367167 | 0.7249804 | 0.9292345 | 0.8220799 | 0.957703 | 1.0112606 | 0.9300578 |
| Phase-1 RCT-189 | 0.7702052 | 1.0880882 | 0.8814318 | 0.9514646 | 0.927505 | 1.03048 | 1.1528964 |
| Phase-1 RCT-179 | 0.76473 | 1.0310601 | 0.971588 | 1.0110725 | 0.8478705 | 0.9959621 | 1.008282 |
| Phase-1 RCT-152 | 0.9983237 | 1.2797565 | 0.9144024 | 0.9934285 | 1.170481 | 1.0005909 | 1.0748382 |
| Phase-1 RCT-123 | 1.2180126 | 0.9825168 | 1.0372825 | 1.1503855 | 1.0392454 | 0.9475712 | 0.9313291 |
| Paraoxonase 1 | 0.5490457 | 1.2321923 | 0.6499275 | 0.8274352 | 0.6301668 | 0.8039061 | 0.8394398 |
| Organic anion transporter 3 | 0.6839926 | 0.8307495 | 1.7660377 | 1.1464902 | 0.990658 | 1.0027584 | 1.0872991 |
| N-hydroxy-2-acetylaminofluorene | 0.4633918 | 0.9007394 | 0.7561622 | 1.0462459 | 0.8401808 | 1.0132732 | 1.0009149 |
| sulfotransferase (ST1C1) | | | | | | | |
| Integrin beta1 | 0.8913387 | 0.8958817 | 1.1434082 | 1.1484933 | 1.1895391 | 1.2759743 | 1.187599 |
| Insulin-like growth factor binding protein 3 | 0.7595621 | 0.7517783 | 1.0250034 | 0.9218206 | 0.8983931 | 0.8151444 | 0.9803886 |
| ID-1 | 1.1348583 | 1.117049 | 0.9896722 | 1.3260568 | 1.201731 | 1.2741591 | 1.1313009 |
| Hepatic lipase | 0.4096699 | 0.7395916 | 0.7741466 | 0.7656857 | 0.6444346 | 0.6378239 | 0.7942272 |
| Heme oxygenase | 0.7656498 | 0.7037601 | 0.9372743 | 0.7161879 | 1.527974 | 1.1091478 | 0.8003252 |
| Equilbrative nitrobenzylthioinosine- | 0.678933 | 0.9716563 | 0.8296835 | 0.9535281 | 0.8981892 | 1.0215355 | 0.9611369 |
| sensitive nucleoside transporter | | | | | | | |
| Epidermal growth factor | 0.9464319 | 1.8327162 | 1.0493588 | 0.9323986 | 1.4311419 | 0.7759705 | 0.9940925 |
| c-myc | 1.4593872 | 0.9192107 | 1.1620953 | 1.0162963 | 1.0749708 | 1.2821244 | 1.010309 |
| Carbonic anhydrase III | 0.2762675 | 1.318189 | 1.2066739 | 1.5381652 | 0.683671 | 1.3674699 | 1.0822979 |
| Beta-actin | 0.5040894 | 0.897674 | 1.0397253 | 1.5636549 | 1.0789865 | 0.9737921 | 0.9482086 |
| Bax (alpha) | 1.1582731 | 1.5667136 | 1.2830487 | 1.1199355 | 1.1929992 | 1.220737 | 1.0056925 |
| Alpha 1 - inhibitor III | 0.3911211 | 0.7079244 | 0.7224569 | 0.6207221 | 0.5719046 | 0.9078235 | 0.8683121 |
| 60S ribosomal protein L6 | 1.0270361 | 0.9957632 | 1.1538827 | 1.175986 | 1.1347727 | 1.1295975 | 1.1544085 |
| Phase-1 RCT-117 | 0.8956635 | 1.69721 | 1.2716464 | 1.0508528 | 1.5117348 | 1.0601634 | 1.2466214 |
| Phase-1 RCT-102 | 0.3089513 | 0.6141785 | 0.867229 | 0.7123037 | 0.4598193 | 0.3358196 | 0.5202518 |
| 3-hydroxyisobutyrate dehydrogenase | 0.7794364 | 1.1583204 | 0.9067104 | 0.8783292 | 0.8507776 | 1.012777 | 1.0932094 |
| ATPase inhibitor (rat mitochondrial IF1 | 0.6261172 | 2.144783 | 0.7523483 | 1.0689827 | 0.9707199 | 0.818203 | 0.7948468 |
| protein) | | | | | | | |
| Alpha-2-macroglobulin | 0.4647911 | 0.9010837 | 0.8063359 | 0.7430179 | 0.6473633 | 0.9903805 | 1.099836 |
| Phase-1 RCT-137 | 0.6980377 | 1.6302003 | 0.7523394 | 0.8213942 | 0.7172529 | 0.7591994 | 0.9208097 |
| Phase-1 RCT 252 | 1.069488 | 1.3014208 | 0.9063108 | 0.8039333 | 0.7522041 | 0.9546151 | 1.0458614 |
| Phase-1 RCT-65 | 1.530546 | 0.8885216 | 1.0492948 | 0.8185152 | 0.9144948 | 1.0865247 | 0.9290935 |
| Liver fatty acid binding protein | 0.3097961 | 0.7477483 | 1.6357622 | 1.4576445 | 1.5821996 | 0.7404268 | 0.915678 |
| Carbamyl phosphate synthetase I | 1.0675259 | 1.4635996 | 0.893522 | 0.8103007 | 0.6998422 | 0.9503316 | 1.0247873 |
| c-jun | 3.097618 | 1.2160735 | 1.1490877 | 0.852124 | 1.1025271 | 1.4643363 | 1.226269 |
| Transthyretin | 0.5726632 | 1.2968193 | 0.6747036 | 0.6038043 | 0.5091327 | 0.5263106 | 0.7030417 |
| Aflatoxin B1 aldehyde reductase | 0.6381856 | 1.3932115 | 0.7014087 | 0.9102977 | 0.7004176 | 0.8301392 | 1.1138501 |
| Phase-1 RCT-15 | 1.3467461 | 1.0281328 | 1.1012775 | 0.925923 | 1.0097433 | 1.0736777 | 0.9869689 |
| Insulin-like growth factor I | 0.5972167 | 0.8088279 | 0.9661401 | 0.8670542 | 0.8927319 | 0.7476081 | 0.8885972 |
| Sodium/bile acid cotransporter | 0.5590594 | 1.4424578 | 0.8587267 | 0.8708533 | 0.7377493 | 0.8389525 | 1.2769556 |
| Organic cation transporter 3 | 0.9381509 | 0.9255762 | 1.0896637 | 1.2236319 | 1.2009283 | 1.1090454 | 1.1381396 |
| Gadd153 | 1.4889789 | 1.5081396 | 1.2519166 | 1.6769141 | 1.1097336 | 1.2237183 | 0.9808785 |
| Phase-1 RCT-109 | 0.8433956 | 0.9243685 | 1.2153028 | 1.4855807 | 1.427764 | 1.441311 | 1.4541881 |
| Phase-1 RCT-88 | 1.2653813 | 1.1898217 | 0.9892768 | 0.8407377 | 0.8947768 | 0.8267574 | 0.9015271 |
| Ubiquitin conjugating enzyme (RAD 6 | 0.8644446 | 1.1891438 | 1.0179031 | 1.2121485 | 0.994929 | 1.0746713 | 1.1622276 |
| homologue) | | | | | | | |
| Glucose-regulated protein 78 | 0.5350605 | 0.9516534 | 1.5092411 | 1.3003453 | 1.643553 | 1.3164022 | 1.1294812 |
| Ribosomal protein L13A | 0.7969471 | 0.9688379 | 1.6368532 | 1.5070733 | 1.5706558 | 1.45864 | 1.3681536 |
| Monocyte chemotactic protein receptor | 1.2790887 | 0.8896768 | 1.0962943 | 1.1872028 | 1.1601523 | 1.0624667 | 1.0464318 |
| (CCR2) | | | | | | | |
| Fas antigen | 1.0026166 | 1.2617052 | 1.1950943 | 1.3758305 | 1.4303813 | 1.2577068 | 1.2590275 |
| Calpactin I heavy chain | 1.1446929 | 0.9332343 | 1.0531199 | 1.2163562 | 1.3901119 | 0.9855546 | 1.0330664 |
| Uncoupling protein 2 | 1.1717832 | 1.0542072 | 1.1697519 | 1.413869 | 1.2624334 | 1.0045985 | 0.9786646 |
| Phase-1 RCT-168 | 0.6138488 | 0.780075 | 0.9761707 | 0.9521159 | 1.1161588 | 1.024106 | 1.0241808 |
| Phase-1 RCT-154 | 2.5475147 | 2.2485664 | 0.956867 | 0.9993966 | 0.9991077 | 0.9834675 | 1.0341693 |
| Superoxide dismutase Mn | 1.0029622 | 1.3913771 | 1.5747628 | 1.5586021 | 1.6861567 | 1.3651761 | 1.2412094 |
| Phase-1 RCT-214 | 0.4055502 | 0.9692238 | 0.9839418 | 0.8448431 | 1.0081819 | 0.5879976 | 0.820463 |
| Phase-1 RCT-225 | 0.6408325 | 0.7091386 | 0.9628665 | 1.1730175 | 1.3434876 | 0.9277262 | 1.0835439 |
| Phase-1 RCT-181 | 0.9410605 | 0.8488927 | 1.0867357 | 1.0406928 | 0.9739859 | 0.8801246 | 0.9130774 |
| Nucleoside diphosphate kinase beta | 1.139818 | 1.7035264 | 1.0044122 | 1.0485855 | 1.0339017 | 0.8011803 | 0.9407457 |
| isoform | | | | | | | |
| Extracellular-signal-regulated kinase 1 | 0.7268254 | 1.288836 | 1.1162939 | 1.2586837 | 0.8366346 | 1.2957016 | 1.0274451 |
| {Ribosomal protein L6} | 1.0734788 | 1.0681065 | 0.9972537 | 1.045468 | 0.9641402 | 1.0742276 | 1.1015782 |
| Aldehyde dehydrogenase, microsomal | 0.7960903 | 0.7675256 | 1.1214892 | 1.02648 | 1.0777656 | 1.0054383 | 1.0876639 |
| p53 | 0.7704963 | 1.0029768 | 0.9513695 | 0.9328778 | 0.9243447 | 0.9817829 | 0.8909785 |
| Phase-1 RCT-239 | 1.1058224 | 0.7795404 | 0.9572584 | 0.7743141 | 0.9774028 | 1.0893745 | 1.0298054 |
| Macrophage inflammatory protein-2 alpha | 1.797691 | 2.1660028 | 1.0764753 | 0.9052038 | 1.1593039 | 0.9518957 | 0.9443452 |
| IgE binding protein | 1.2985214 | 0.8752174 | 1.0462023 | 1.248985 | 1.1920556 | 0.9353232 | 0.95473 |
| Phase-1 RCT-205 | 0.9298396 | 0.7869697 | 0.9418964 | 1.0257899 | 0.9438863 | 0.9985455 | 1.0079683 |
| Melanoma-associated antigen ME491 | 1.1373084 | 1.004523 | 0.9736744 | 1.6206717 | 1.1098901 | 1.0102526 | 1.1262056 |
| Phase-1 RCT-242 | 1.8894325 | 0.951277 | 0.9583244 | 1.0463378 | 0.9751728 | 1.1688443 | 1.0017463 |
| Enolase alpha | 0.8716074 | 2.3281786 | 1.0378855 | 0.9762643 | 1.0826788 | 0.8477321 | 0.8186911 |
| Thymosin beta-10 | 0.8322679 | 1.2984791 | 1.097345 | 1.5103061 | 1.1944747 | 1.1192976 | 1.1449518 |
| Cofilin | 1.1505121 | 1.1726911 | 0.757438 | 0.8700941 | 0.8563129 | 0.8880473 | 1.0303377 |
| Carbonyl reductase | 1.2126508 | 0.9468309 | 1.0316017 | 1.1252222 | 1.0064368 | 1.2229317 | 0.9782471 |
| MAP kinase kinase | 0.7756838 | 0.7947748 | 1.0492274 | 1.0468414 | 1.0725509 | 1.0714064 | 0.9695403 |
| Phase-1 RCT-162 | 1.0870209 | 1.1600704 | 1.0504576 | 1.0845264 | 1.003322 | 1.0049698 | 0.9999999 |
| Phase-1 RCT-72 | 0.9191091 | 0.9388089 | 0.9754179 | 0.9330528 | 0.9865277 | 0.9291911 | 0.9371134 |
| Ribosomal protein S8 | 1.0035172 | 1.5685288 | 1.0577986 | 1.2118728 | 1.0091369 | 1.0089744 | 1.0726746 |
| Proliferating cell nuclear antigen gene | 0.9965306 | 1.073584 | 0.985312 | 1.2164209 | 0.9717702 | 0.9817861 | 0.8563434 |
| Phase-1 RCT-192 | 1.0868511 | 1.3035669 | 1.2245295 | 1.1848744 | 1.0970803 | 0.9716311 | 1.0871353 |
| IkB-a | 0.8979037 | 1.2738705 | 0.9704366 | 1.0312173 | 0.8759286 | 1.026036 | 0.9553378 |
| Endogenous retroviral sequence, 5′ and | 0.7270998 | 0.9786004 | 1.2685239 | 1.4342458 | 1.1309975 | 1.309439 | 1.3024776 |
| 3′ LTR | | | | | | | |
| Phase-1 RCT-37 | 1.0284132 | 1.0003376 | 1.073987 | 1.0592281 | 1.0531334 | 1.0522472 | 1.0773978 |
| Matrix metalloproteinase-1 | 0.9930557 | 1.6675236 | 0.9368548 | 0.8786496 | 0.877143 | 0.9281852 | 0.9691064 |
| Cyclin G | 5.5452766 | 3.072167 | 1.1616812 | 1.7081167 | 1.1832772 | 1.1340841 | 1.041701 |
| DNA polymerase beta | 0.9863183 | 1.7564968 | 0.95522 | 0.9724148 | 0.9051448 | 0.7428951 | 0.9002935 |
| Phase-1 RCT-127 | 1.0762155 | 1.1973124 | 1.1409795 | 1.1414555 | 1.2466059 | 1.0917267 | 1.0629215 |
| Multidrug resistant protein-2 | 4.7353415 | 2.4912171 | 1.091319 | 0.8236207 | 1.0885489 | 1.2430158 | 1.1257577 |
| Ribosomal protein S9 | 0.9079319 | 1.432359 | 0.9923761 | 1.127075 | 0.9874634 | 1.0236433 | 1.058497 |
| Phase-1 RCT-12 | 1.0409336 | 0.9786567 | 1.1672189 | 0.9478773 | 0.9573671 | 1.1591169 | 1.0141681 |
| Beta-tubulin, class I | 0.8217132 | 1.1047298 | 1.1848013 | 0.887288 | 0.9590552 | 1.1541145 | 0.8899295 |
| Argininosuccinate lyase | 1.534673 | 0.551445 | 0.9187176 | 0.8658473 | 0.9176588 | 1.3101095 | 1.2478269 |
| Elongation factor-1 alpha | 0.8813723 | 1.4793574 | 0.8932042 | 0.8690593 | 0.8515013 | 0.9249798 | 0.946063 |
| Phase-1 RCT-55 | 0.761299 | 0.7365172 | 1.0657697 | 2.724562 | 0.9669369 | 0.8310602 | 0.8766686 |
| Macrophage inflammatory protein-1 alpha | 2.6966367 | 0.9792271 | 1.0316312 | 0.8831336 | 0.9187422 | 1.0564429 | 0.9925865 |
| c-H-ras | 0.9425293 | 1.2545226 | 1.1742551 | 1.0280901 | 1.405547 | 1.0569907 | 0.9770325 |
| Multidrug resistant protein-1 | 6.6054926 | 2.7571132 | 1.1871798 | 0.742881 | 0.999639 | 1.1283089 | 1.1449374 |
| T-cell cyclophilin | 0.8504638 | 1.8998427 | 0.9081715 | 0.9737377 | 0.8821054 | 0.7008191 | 0.8608836 |
| Phase-1 RCT-185 | 0.6217701 | 1.1922091 | 0.6888641 | 0.7358791 | 0.7176532 | 0.7073047 | 0.8661876 |
| 25-DX | 0.714602 | 1.3535051 | 1.1197815 | 1.2259307 | 1.1285962 | 1.1927092 | 1.2424228 |
| Ref-1 | 1.1125329 | 1.2221881 | 1.6313797 | 0.9953124 | 1.0078123 | 1.0381919 | 1.1855222 |
| Apolipoprotein CIII | 0.8242241 | 1.525113 | 1.1189816 | 1.125669 | 1.3099873 | 1.1846015 | 1.1178908 |
| NADPH quinone oxidoreductase-1 (DT- | 1.2557561 | 1.4717712 | 2.0343647 | 1.1834509 | 0.950388 | 1.0827268 | 1.2913818 |
| diaphorase) | | | | | | | |
| Pyruvate kinase, muscle | 0.8119376 | 1.0635122 | 1.1544617 | 1.6451802 | 0.9138776 | 0.9254126 | 0.9065152 |
| | CLO | CLOZ | CLOZ | CLOZ | CLOZ | CLOZ | CLOZ |
| Compound-Dose (2) | 250 | 45 | 45 | 45 | 180 | 180 | 180 |
| Animal Number (3) | 1846 | 2424 | 2425 | 2426 | 2434 | 2435 | 2436 |
| Liver Toxicity Necrosis Classification (4) | no | no | no | no | no | no | no |
| Gene Name (5) | | | | | | | |
| Phase-1 RCT-78 | 0.9921061 | 0.9653634 | 1.0674027 | 0.9788164 | 1.0018809 | 0.8982704 | 0.8865598 |
| Gamma-actin, cytoplasmic | 0.6459996 | 0.886831 | 0.7804518 | 0.7148457 | 0.9918829 | 0.8274213 | 0.7739847 |
| Matrin F/G | 1.0064962 | 1.304306 | 1.328683 | 1.2367427 | 1.3119333 | 1.0637772 | 0.9590906 |
| Zinc finger protein | 1.0017802 | 0.9231237 | 0.7508053 | 0.9783699 | 0.8219812 | 0.9306771 | 0.8928176 |
| Cathepsin L, sequence 2 | 0.8837841 | 1.2393463 | 1.2705245 | 1.1856972 | 1.1163008 | 1.2032956 | 0.9103449 |
| Gadd45 | 0.988731 | 1.1427332 | 1.0090585 | 1.0248095 | 1.4100426 | 1.2979765 | 1.8108071 |
| Phase-1 RCT-144 | 1.2672701 | 0.8549211 | 0.8348357 | 0.9015942 | 0.9811293 | 0.908677 | 0.955141 |
| Phase-1 RCT-145 | 1.3609823 | 0.8974987 | 0.867854 | 0.9413139 | 0.9152613 | 0.945638 | 0.9147869 |
| Phase-1 RCT-50 | 1.2561848 | 0.860321 | 0.8624685 | 0.9993298 | 1.0326535 | 1.0469033 | 0.9903552 |
| Phase-1 RCT-92 | 0.9808787 | 1.0616745 | 0.9555532 | 0.9127119 | 1.124379 | 0.9706637 | 0.9424857 |
| Phase-1 RCT-89 | 0.9596862 | 1.102487 | 1.0727208 | 0.9967388 | 1.1413121 | 0.9235765 | 0.9075318 |
| 14-3-3 zeta | 1.0137153 | 1.1270578 | 1.1470114 | 0.9113008 | 0.9559349 | 0.8098114 | 0.7541698 |
| Dynamin-1 (D100) | 1.1738801 | 1.023684 | 0.9301965 | 0.9578129 | 0.9023999 | 0.8788517 | 0.7925122 |
| Insulin-like growth factor binding protein 1 | 1.1587747 | 1.0503203 | 1.0420595 | 0.9399124 | 1.4654089 | 1.2531165 | 1.5121571 |
| L-gulono-gamma-lactone oxidase | 0.8772922 | 1.3819056 | 1.5457462 | 1.3697044 | 1.2005341 | 0.9574766 | 1.0319285 |
| Ornithine decarboxylase | 0.8452814 | 1.1223097 | 1.1905354 | 1.1402204 | 1.1084533 | 1.2060008 | 1.3621129 |
| PAR interacting protein | 1.086781 | 0.8853049 | 0.8669899 | 0.972051 | 1.0335934 | 0.9398502 | 0.9906902 |
| Phase-1 RCT-128 | 1.0427474 | 1.3032243 | 1.0724229 | 0.9624687 | 0.869736 | 0.7495271 | 0.6872599 |
| Phase-1 RCT-180 | 0.8617662 | 0.8242607 | 0.9234825 | 0.9418231 | 0.6705864 | 0.8414683 | 0.8774861 |
| Phase-1 RCT-182 | 0.8593107 | 1.2236092 | 1.0119786 | 1.0263534 | 1.093086 | 1.0522426 | 1.3565948 |
| Phase-1 RCT-207 | 1.4897561 | 0.812847 | 0.8553222 | 0.9894857 | 0.8592435 | 0.9296542 | 0.835365 |
| Phase-1 RCT-213 | 1.0384609 | 0.9716977 | 0.9187113 | 0.9880305 | 0.9102739 | 1.0202829 | 0.964093 |
| Phase-1 RCT-256 | 1.0067914 | 1.1925324 | 1.2427804 | 1.1180465 | 1.05008 | 0.8061342 | 0.7603708 |
| Phase-1 RCT-258 | 1.1854497 | 1.0078565 | 0.8844186 | 1.024528 | 0.9641455 | 0.985597 | 0.9303849 |
| Phase-1 RCT-264 | 0.6123741 | 1.1039298 | 1.1797496 | 0.9400041 | 0.983529 | 0.6579118 | 0.6065717 |
| Phase-1 RCT-271 | 0.9471153 | 1.2264541 | 1.0611937 | 0.9930724 | 0.9926574 | 1.0380732 | 0.9008862 |
| Phase-1 RCT-288 | 0.9349029 | 1.0989327 | 1.0391032 | 1.0451883 | 0.9830022 | 0.9895406 | 0.8829587 |
| Phase-1 RCT-38 | 0.9772005 | 1.2382972 | 1.2852923 | 1.168362 | 1.0336496 | 0.8561926 | 0.8183252 |
| Phase-1 RCT-39 | 1.2472459 | 0.8956333 | 0.8312525 | 0.9610307 | 1.125456 | 1.1323239 | 1.0077417 |
| Phase-1 RCT-68 | 1.0663778 | 0.8381461 | 0.9717728 | 0.9977662 | 1.1153136 | 1.0944927 | 1.0418516 |
| Phase-1 RCT-33 | 1.025996 | 1.220438 | 1.2245404 | 1.0739689 | 1.2708685 | 1.0056202 | 1.3127906 |
| Phase-1 RCT-36 | 1.1394242 | 0.8996747 | 0.8930224 | 0.9837513 | 1.1013166 | 1.0100023 | 1.1851918 |
| 17-beta hydroxysteroid dehydrogenase, | 1.0694108 | 1.5687214 | 1.8456241 | 1.4490196 | 1.936508 | 0.8198347 | 1.3169792 |
| type 2 | | | | | | | |
| Senescence marker protein-30 | 0.9643821 | 0.9815532 | 1.2791436 | 1.129092 | 0.934338 | 0.8848776 | 1.3212239 |
| Ribosomal protein S17 | 0.821361 | 1.0750471 | 1.1589347 | 1.0278233 | 1.0485919 | 1.1580966 | 0.918547 |
| Phase-1 RCT-83 | 1.0399531 | 1.0628535 | 0.8176921 | 0.9137351 | 0.7876189 | 0.9013009 | 0.9108204 |
| Phase-1 RCT-49 | 1.39848 | 0.8498006 | 0.874466 | 0.9788483 | 1.0009714 | 0.9330128 | 0.9872036 |
| Phase-1 RCT-48 | 0.8020226 | 1.0534043 | 0.95551 | 0.9435244 | 0.8895587 | 0.72537 | 0.811059 |
| Phase-1 RCT-40 | 0.6756692 | 1.2148898 | 1.1878873 | 1.0718266 | 1.1340768 | 0.9319364 | 0.7870088 |
| Phase-1 RCT-296 | 0.7381998 | 1.4932228 | 1.3967603 | 1.2012094 | 1.6095632 | 1.0124761 | 1.0056144 |
| Phase-1 RCT-295 | 0.9161142 | 1.1796182 | 1.1108142 | 1.0437163 | 1.1446435 | 1.0401138 | 1.0004104 |
| Phase-1 RCT-291 | 0.7735763 | 1.3218302 | 1.2123888 | 1.0946348 | 1.0536492 | 1.0663463 | 0.9659656 |
| Phase-1 RCT-270 | 0.8101968 | 1.1915478 | 1.0854709 | 0.9756414 | 1.0210506 | 0.871088 | 0.9497725 |
| Phase-1 RCT-241 | 1.1946778 | 0.9616112 | 0.8852473 | 0.9528104 | 0.9196029 | 1.1230364 | 1.3017248 |
| Phase-1 RCT-191 | 1.0522951 | 1.0814689 | 1.059468 | 1.0778302 | 1.0870003 | 1.1445812 | 1.1546031 |
| Phase-1 RCT-189 | 0.8936796 | 1.2841735 | 1.1639428 | 1.0744107 | 1.0410655 | 0.7351005 | 0.831108 |
| Phase-1 RCT-179 | 0.8879474 | 1.186585 | 1.0847381 | 0.9634397 | 1.0190275 | 0.868883 | 0.8332234 |
| Phase-1 RCT-152 | 0.8997506 | 1.0202174 | 1.2492963 | 1.1147162 | 1.1816701 | 1.0386245 | 1.1438717 |
| Phase-1 RCT-123 | 1.1763302 | 0.9552969 | 0.796243 | 0.9366691 | 1.0087385 | 1.0162578 | 0.9883124 |
| Paraoxonase 1 | 0.7364444 | 1.124285 | 1.3163103 | 1.0480409 | 1.2633115 | 0.8438304 | 1.0580598 |
| Organic anion transporter 3 | 1.2428385 | 1.28996 | 1.0508424 | 1.2768179 | 1.3698672 | 1.2174009 | 0.9871002 |
| N-hydroxy-2-acetylaminofluorene | 0.9120821 | 1.2311027 | 1.2570467 | 1.1288201 | 0.6966333 | 0.9997854 | 0.9228205 |
| sulfotransferase (ST1C1) | | | | | | | |
| Integrin beta1 | 1.3816445 | 0.9876833 | 1.1289806 | 1.0033833 | 1.1523081 | 1.042288 | 0.9851696 |
| Insulin-like growth factor binding protein 3 | 0.7420601 | 0.8539061 | 1.2179834 | 1.0362505 | 0.8317886 | 0.7508972 | 0.7019985 |
| ID-1 | 1.2305789 | 0.7822036 | 0.9563868 | 1.0813543 | 0.9087713 | 0.7822133 | 0.7844114 |
| Hepatic lipase | 0.7132315 | 0.8971055 | 1.0274721 | 0.7982159 | 0.9622249 | 0.4426219 | 0.7400179 |
| Heme oxygenase | 1.0428406 | 0.8955033 | 0.8950648 | 0.9139635 | 1.1075792 | 0.9360811 | 1.3717853 |
| Equilbrative nitrobenzylthioinosine- | 0.9915086 | 1.0761796 | 1.0090948 | 0.9594897 | 0.8143289 | 1.0519668 | 1.034184 |
| sensitive nucleoside transporter | | | | | | | |
| Epidermal growth factor | 0.7275533 | 0.9753428 | 1.063766 | 1.0019679 | 1.0069776 | 1.0977278 | 1.0249517 |
| c-myc | 1.0064795 | 0.8243019 | 0.7078105 | 0.9015688 | 0.8670703 | 0.9332053 | 0.866055 |
| Carbonic anhydrase III | 1.32104 | 1.8393288 | 0.8379002 | 1.200754 | 0.6359521 | 0.9732239 | 0.6005103 |
| Beta-actin | 0.6332676 | 1.2093507 | 0.8156328 | 0.6453843 | 0.7704772 | 0.6727369 | 0.426125 |
| Bax (alpha) | 1.0377324 | 0.842567 | 0.9716278 | 1.0921773 | 0.9540634 | 1.0125102 | 1.0886308 |
| Alpha 1 - inhibitor III | 0.7781227 | 0.9053752 | 1.4565855 | 1.1119854 | 1.1113497 | 0.5575254 | 0.7340002 |
| 60S ribosomal protein L6 | 0.8401233 | 1.1784494 | 0.9274687 | 0.948593 | 0.8960638 | 1.2027582 | 1.0619768 |
| Phase-1 RCT-117 | 1.0893842 | 1.1531562 | 1.1112852 | 1.1396813 | 0.8124891 | 0.8045102 | 0.9560958 |
| Phase-1 RCT-102 | 0.6676589 | 0.6473121 | 0.8773212 | 0.853246 | 0.470931 | 0.4268305 | 0.3359523 |
| 3-hydroxyisobutyrate dehydrogenase | 0.8548204 | 1.5416944 | 1.2398165 | 1.1053338 | 1.1814077 | 1.0193034 | 1.042428 |
| ATPase inhibitor (rat mitochondrial IF1 | 0.5887893 | 1.2030883 | 1.0725507 | 1.0740273 | 0.7631808 | 0.8401004 | 0.8556082 |
| protein) | | | | | | | |
| Alpha-2-macroglobulin | 0.9263381 | 0.7946413 | 0.9460717 | 1.0827864 | 0.9700276 | 1.1778711 | 1.5338515 |
| Phase-1 RCT-137 | 0.6965868 | 1.2128456 | 0.8848574 | 0.8659195 | 0.8203449 | 0.7703918 | 0.9451094 |
| Phase-1 RCT 252 | 0.8962863 | 1.42121 | 1.0121545 | 1.0463375 | 1.0745214 | 0.7418699 | 0.7667341 |
| Phase-1 RCT-65 | 1.0594393 | 1.0842564 | 1.2085905 | 1.0568321 | 1.1224254 | 0.9149387 | 0.935248 |
| Liver fatty acid binding protein | 0.5498661 | 0.9574943 | 1.0121646 | 0.8571658 | 0.8608677 | 0.7698577 | 0.4714811 |
| Carbamyl phosphate synthetase I | 0.8455644 | 1.5860901 | 1.3347597 | 1.1294214 | 1.1664128 | 0.78521 | 0.8459566 |
| c-jun | 1.1390681 | 1.5758096 | 0.969397 | 1.1066635 | 1.3872062 | 0.8922199 | 1.133416 |
| Transthyretin | 0.51361 | 0.8692275 | 1.3682896 | 0.9804305 | 0.9502858 | 0.6537179 | 0.6914399 |
| Aflatoxin B1 aldehyde reductase | 0.8669881 | 1.0157241 | 1.0497903 | 1.1489407 | 0.9575507 | 1.3686484 | 1.0340062 |
| Phase-1 RCT-15 | 0.9506648 | 1.1280242 | 1.0003583 | 1.1127989 | 1.3357984 | 1.099218 | 1.0322719 |
| Insulin-like growth factor I | 0.6571685 | 0.932158 | 1.2666804 | 1.1426656 | 0.7593854 | 0.5065861 | 0.5353174 |
| Sodium/bile acid cotransporter | 0.7423678 | 1.1460938 | 1.3909429 | 1.32358 | 1.1055479 | 1.1638454 | 0.9677767 |
| Organic cation transporter 3 | 0.9532456 | 1.0669408 | 0.9503462 | 0.9414581 | 0.9064956 | 1.0379571 | 1.0734189 |
| Gadd153 | 1.0080427 | 0.8778138 | 1.0093688 | 1.0837454 | 1.0934826 | 1.049213 | 1.096267 |
| Phase-1 RCT-109 | 1.15212 | 1.0035005 | 0.9705471 | 1.0784366 | 0.9549983 | 0.9468043 | 0.8308208 |
| Phase-1 RCT-88 | 1.2359864 | 0.9088665 | 0.9458805 | 1.0276941 | 1.3625522 | 1.1468865 | 1.0369705 |
| Ubiquitin conjugating enzyme (RAD 6 | 0.8982192 | 1.0793881 | 0.9609292 | 0.8917644 | 0.8108932 | 0.9383767 | 1.0173624 |
| homologue) | | | | | | | |
| Glucose-regulated protein 78 | 1.0579383 | 1.2491922 | 1.1877414 | 1.1735767 | 1.0445507 | 1.1820309 | 1.1505797 |
| Ribosomal protein L13A | 1.057396 | 1.0106195 | 1.08023 | 1.0932258 | 0.9440916 | 0.915589 | 0.8081462 |
| Monocyte chemotactic protein receptor | 1.1726363 | 0.8493856 | 0.8416292 | 1.0206528 | 0.8926773 | 1.0865068 | 1.0995505 |
| (CCR2) | | | | | | | |
| Fas antigen | 1.0241503 | 0.9021689 | 0.991493 | 0.9681429 | 0.9221914 | 1.0334537 | 0.891094 |
| Calpactin I heavy chain | 1.1012253 | 0.8790943 | 0.8905688 | 1.0159656 | 0.9645357 | 1.0714376 | 0.9879699 |
| Uncoupling protein 2 | 0.9015287 | 0.8727165 | 0.8961616 | 0.9260189 | 0.7549052 | 0.8144826 | 0.7055593 |
| Phase-1 RCT-168 | 0.9078201 | 0.937528 | 0.9982292 | 0.9695318 | 1.1368818 | 1.0466332 | 1.0800414 |
| Phase-1 RCT-154 | 1.2637999 | 0.9103072 | 0.8596345 | 0.9527857 | 0.9199665 | 1.0494719 | 0.9609252 |
| Superoxide dismutase Mn | 1.097916 | 1.2434553 | 1.2872769 | 1.2595822 | 1.101772 | 1.4288327 | 1.1436363 |
| Phase-1 RCT-214 | 0.6527693 | 1.0036073 | 0.9534135 | 0.8544126 | 0.9200357 | 0.9650487 | 1.004252 |
| Phase-1 RCT-225 | 1.0387151 | 1.4208792 | 0.8867768 | 1.0463276 | 1.050487 | 0.7986276 | 0.6394028 |
| Phase-1 RCT-181 | 1.0489882 | 1.0521691 | 1.0441566 | 1.0882947 | 1.1397661 | 0.9695453 | 1.0076723 |
| Nucleoside diphosphate kinase beta | 0.775411 | 1.007616 | 1.1206173 | 1.0439776 | 1.1101897 | 1.3020135 | 1.2315216 |
| isoform | | | | | | | |
| Extracellular-signal-regulated kinase 1 | 1.11061 | 1.2805088 | 0.8534953 | 1.095857 | 0.6704319 | 0.9842781 | 0.6232093 |
| {Ribosomal protein L6} | 0.8919665 | 1.2610809 | 1.2469453 | 1.1794071 | 1.2498722 | 1.085636 | 1.2129378 |
| Aldehyde dehydrogenase, microsomal | 0.8521644 | 1.1041787 | 1.0348518 | 0.9244143 | 0.8684579 | 0.9102538 | 1.0100766 |
| p53 | 1.0433538 | 0.8716977 | 0.9243879 | 0.8836221 | 0.8864961 | 0.8172711 | 0.8590676 |
| Phase-1 RCT-239 | 1.3403263 | 0.7752677 | 0.8423 | 0.9090232 | 0.9022249 | 0.8250873 | 0.7299355 |
| Macrophage inflammatory protein-2 alpha | 1.044781 | 0.7423833 | 0.8488313 | 1.0639075 | 1.0813316 | 1.2070769 | 1.230198 |
| IgE binding protein | 1.0180311 | 0.9892967 | 0.9474773 | 0.9831421 | 0.9561929 | 1.033856 | 0.9813864 |
| Phase-1 RCT-205 | 1.2848107 | 1.0223074 | 0.9144027 | 1.0809205 | 1.0888368 | 1.1281394 | 1.1073841 |
| Melanoma-associated antigen ME491 | 1.2460276 | 0.8959557 | 0.7721415 | 0.7758122 | 0.8356031 | 0.8061376 | 0.9347186 |
| Phase-1 RCT-242 | 1.7604536 | 0.8236116 | 0.7435682 | 0.8693325 | 1.1394132 | 0.795703 | 0.8916928 |
| Enolase alpha | 0.7416872 | 1.0390514 | 1.2987049 | 1.0463995 | 1.2012522 | 0.8717675 | 1.1920075 |
| Thymosin beta-10 | 1.0264677 | 0.9138593 | 0.8822638 | 0.9540584 | 0.7770349 | 0.8465763 | 0.7005094 |
| Cofilin | 0.8334626 | 1.0615832 | 0.9992583 | 0.9019377 | 0.9327692 | 0.8491512 | 0.8987105 |
| Carbonyl reductase | 1.0018888 | 0.8249927 | 0.8177791 | 0.9332739 | 0.8836972 | 1.1429694 | 1.263531 |
| MAP kinase kinase | 0.8948286 | 0.8841588 | 0.9968426 | 1.0419743 | 1.073772 | 1.0733225 | 1.0155474 |
| Phase-1 RCT-162 | 0.9301015 | 0.9909073 | 0.9979786 | 1.030518 | 1.0753801 | 1.0683684 | 0.9374762 |
| Phase-1 RCT-72 | 1.4278687 | 0.7916698 | 0.8000569 | 0.9107598 | 0.9746472 | 0.9566778 | 0.9987635 |
| Ribosomal protein S8 | 0.8102557 | 1.1661334 | 1.2827874 | 1.1929779 | 1.2079232 | 1.1315039 | 1.1351981 |
| Proliferating cell nuclear antigen gene | 1.0428165 | 6.8335965 | 0.957534 | 1.0210738 | 0.9756644 | 0.9025084 | 0.965236 |
| Phase-1 RCT-192 | 0.8666282 | 6.8728432 | 0.7992523 | 0.8650084 | 1.0478005 | 0.9789699 | 0.8958922 |
| IkB-a | 0.9531507 | 0.8280283 | 1.1372018 | 1.0238105 | 1.1712449 | 0.9125757 | 1.014124 |
| Endogenous retroviral sequence, 5′ and | 1.161104 | 1.4116328 | 0.9164059 | 1.5041084 | 0.9510433 | 0.7285916 | 0.7010502 |
| 3′ LTR | | | | | | | |
| Phase-1 RCT-37 | 0.9569738 | 1.0998311 | 1.0055625 | 1.0144075 | 1.1097153 | 1.0188518 | 1.0264846 |
| Matrix metalloproteinase-1 | 0.7707374 | 1.0801774 | 1.286742 | 1.0710963 | 1.1271043 | 1.0787076 | 0.8671973 |
| Cyclin G | 1.3977548 | 0.9148868 | 0.9581808 | 1.0044901 | 1.0929897 | 1.0835636 | 1.1368802 |
| DNA polymerase beta | 0.7056531 | 0.9564673 | 1.0060097 | 0.9620535 | 0.9958712 | 1.0786865 | 1.1102712 |
| Phase-1 RCT-127 | 1.1384555 | 0.9136111 | 0.9259425 | 0.9668784 | 0.8966954 | 0.8966689 | 0.9835529 |
| Multidrug resistant protein-2 | 1.247048 | 0.9946356 | 1.1621568 | 1.1561549 | 1.5246601 | 1.1736935 | 1.0238371 |
| Ribosomal protein S9 | 0.8948858 | 1.0470507 | 0.9134198 | 0.8716263 | 0.8005883 | 0.8972151 | 1.0072577 |
| Phase-1 RCT-12 | 1.0797205 | 0.9611117 | 0.8989689 | 0.9765223 | 1.0831896 | 0.9951761 | 1.1291821 |
| Beta-tubulin, class I | 0.768421 | 1.2084737 | 1.0185635 | 1.0266479 | 1.107597 | 1.0901651 | 1.1788443 |
| Argininosuccinate lyase | 1.0149401 | 1.5628184 | 1.4103422 | 1.3624986 | 1.2824941 | 0.9230627 | 0.9817164 |
| Elongation factor-1 alpha | 0.7477521 | 1.1752241 | 1.3037202 | 1.0361601 | 1.1349288 | 0.8953901 | 0.8066633 |
| Phase-1 RCT-55 | 1.1409619 | 0.7669758 | 0.8207182 | 0.8844903 | 0.9054865 | 0.9470471 | 0.8631391 |
| Macrophage inflammatory protein-1 alpha | 0.9763697 | 0.8088356 | 0.7538148 | 1.0188313 | 0.7611294 | 1.2754446 | 1.118549 |
| c-H-ras | 0.8015127 | 0.993975 | 0.8953718 | 0.9794278 | 0.9648464 | 1.0844992 | 1.1016494 |
| Multidrug resistant protein-1 | 1.1179422 | 1.0488818 | 1.1886497 | 1.217331 | 1.4404448 | 1.2396317 | 1.1010787 |
| T-cell cyclophilin | 0.6536596 | 0.9730754 | 1.0262281 | 0.9181312 | 0.923575 | 0.9845122 | 1.0534306 |
| Phase-1 RCT-185 | 0.7179268 | 1.1407553 | 1.1132104 | 0.9824638 | 0.9882316 | 0.764919 | 0.9358397 |
| 25-DX | 0.9575113 | 1.24935 | 1.128952 | 1.1130946 | 0.8009916 | 1.2993685 | 0.9168872 |
| Ref-1 | 1.0384986 | 1.0354985 | 1.0446534 | 1.1255326 | 1.0803958 | 1.2402488 | 1.6018965 |
| Apolipoprotein CIII | 1.2709482 | 1.2015684 | 1.0319034 | 1.0470655 | 0.8604853 | 0.6822078 | 0.7708933 |
| NADPH quinone oxidoreductase-1 (DT- | 1.0170316 | 1.7166524 | 1.4238293 | 1.7563319 | 1.5783402 | 1.6318972 | 2.9773736 |
| diaphorase) | | | | | | | |
| Pyruvate kinase, muscle | 0.915594 | 1.0343441 | 0.9291596 | 0.9898635 | 1.1143997 | 1.0949875 | 0.9207206 |
| | CMC | CMC | CMC | CHEX | CHEX | CHEX | CHEX |
| Compound-Dose (2) | 30 | 30 | 30 | 0.5 | 0.5 | 0.5 | 2 |
| Animal Number (3) | 154 | 155 | 156 | 2244 | 2245 | 2246 | 2254 |
| Liver Toxicity Necrosis Classification (4) | no | no | no | no | no | no | no |
| Gene Name (5) | | | | | | | |
| Phase-1 RCT-78 | 1.0471661 | 0.8371602 | 0.9030145 | 1.0157197 | 0.8898794 | 0.8257909 | 0.6988697 |
| Gamma-actin, cytoplasmic | 0.9099355 | 1.1920847 | 0.7598121 | 0.9983807 | 1.5683876 | 1.1129632 | 3.6529722 |
| Matrin F/G | 0.9423861 | 0.7511268 | 0.8743944 | 1.4549469 | 1.4021835 | 0.9668484 | 1.1528054 |
| Zinc finger protein | 1.0799911 | 1.0614225 | 1.1600953 | 0.932975 | 0.8470814 | 0.925186 | 1.0181242 |
| Cathepsin L, sequence 2 | 0.8006905 | 0.8591436 | 0.720271 | 1.0308439 | 1.2493106 | 1.0159839 | 2.0817847 |
| Gadd45 | 0.8124576 | 0.7252486 | 0.8677296 | 1.1025003 | 0.7626423 | 0.9921554 | 0.8955244 |
| Phase-1 RCT-144 | 1.0251744 | 1.0935436 | 1.0946478 | 0.9016562 | 1.1117104 | 1.1677814 | 1.6792383 |
| Phase-1 RCT-145 | 1.0227491 | 1.1880487 | 1.0696088 | 0.9462535 | 1.0961993 | 1.2302104 | 1.561673 |
| Phase-1 RCT-50 | 1.1839706 | 0.9406687 | 1.1693006 | 0.9058889 | 0.8851095 | 0.9622647 | 1.407187 |
| Phase-1 RCT-92 | 0.7277244 | 0.859071 | 1.0213327 | 0.997039 | 0.915869 | 0.7397689 | 0.7992213 |
| Phase-1 RCT-89 | 0.8507434 | 0.9709819 | 0.8674203 | 1.0565872 | 0.9712536 | 0.8421634 | 0.7941308 |
| 14-3-3 zeta | 1.1341782 | 0.9956855 | 1.0426875 | 0.9306471 | 1.0259876 | 0.7577298 | 1.1433519 |
| Dynamin-1 (D100) | 1.0326974 | 1.1042852 | 1.0625716 | 0.8478524 | 0.9891286 | 0.840886 | 0.9532344 |
| Insulin-like growth factor binding protein 1 | 1.2017453 | 1.1822572 | 1.3050629 | 1.3025428 | 1.0495468 | 1.0004839 | 1.1440232 |
| L-gulono-gamma-lactone oxidase | 0.7435418 | 0.7543758 | 1.0636863 | 0.6225383 | 0.57385 | 0.2932492 | 0.4313738 |
| Ornithine decarboxylase | 1.1020507 | 1.1636727 | 1.1864347 | 0.9590167 | 0.7149722 | 1.210663 | 1.7995795 |
| PAR interacting protein | 0.9775385 | 1.0541832 | 0.9468072 | 0.9880509 | 1.2141242 | 1.366236 | 1.910845 |
| Phase-1 RCT-128 | 0.8832258 | 1.1688087 | 0.9260169 | 0.8660524 | 1.6040848 | 0.7257264 | 0.9307375 |
| Phase-1 RCT-180 | 0.9671595 | 1.1545066 | 1.0576249 | 0.9003779 | 1.1296495 | 0.9405158 | 2.0179589 |
| Phase-1 RCT-182 | 0.9692158 | 0.9014889 | 0.8570439 | 1.0740895 | 0.9739921 | 0.777604 | 0.9362958 |
| Phase-1 RCT-207 | 1.1986428 | 0.9822658 | 1.1432531 | 0.9528819 | 1.3641334 | 1.5906587 | 2.3335903 |
| Phase-1 RCT-213 | 1.0586464 | 1.1866939 | 1.0921637 | 1.0683076 | 1.4074974 | 1.6072657 | 1.7257162 |
| Phase-1 RCT-256 | 0.7614757 | 0.8570786 | 0.7664978 | 1.0104756 | 1.067425 | 0.7722384 | 1.3992337 |
| Phase-1 RCT-258 | 1.0402327 | 1.0994966 | 1.020244 | 0.9739963 | 1.0581328 | 1.0956938 | 1.3866968 |
| Phase-1 RCT-264 | 0.9438748 | 0.8127987 | 0.9459364 | 0.7729994 | 0.5210472 | 0.516007 | 0.4800665 |
| Phase-1 RCT-271 | 0.9587255 | 0.9496664 | 0.9578117 | 0.9253091 | 0.9352302 | 0.5926202 | 0.9161555 |
| Phase-1 RCT-288 | 1.2338227 | 1.2624696 | 1.1103075 | 0.9228277 | 0.8750953 | 0.8584068 | 0.9487939 |
| Phase-1 RCT-38 | 0.791501 | 0.8914421 | 0.7562524 | 1.0260766 | 1.1277264 | 0.8149691 | 1.4826338 |
| Phase-1 RCT-39 | 0.9760744 | 0.9951084 | 0.9268434 | 1.1249387 | 0.9459689 | 1.190913 | 1.2011948 |
| Phase-1 RCT-68 | 1.0819287 | 1.0549021 | 1.0792909 | 0.9752845 | 1.0317582 | 1.0428107 | 1.2016842 |
| Phase-1 RCT-33 | 0.8300129 | 1.0228612 | 0.8510871 | 0.9114527 | 0.9435144 | 0.9022719 | 1.4121661 |
| Phase-1 RCT-36 | 1.0125273 | 1.0163101 | 0.9019474 | 0.8754864 | 0.7918165 | 0.8005832 | 0.9895583 |
| 17-beta hydroxysteroid dehydrogenase, | 0.3596137 | 0.6680751 | 1.0361084 | 0.7569813 | 0.6426736 | 0.6781303 | 0.8938599 |
| type 2 | | | | | | | |
| Senescence marker protein-30 | 0.6514168 | 0.7213361 | 0.8255357 | 0.8981316 | 0.6800154 | 0.7424635 | 0.6015016 |
| Ribosomal protein S17 | 1.2249691 | 1.2911468 | 1.1810311 | 1.2319673 | 2.198811 | 2.3979373 | 3.8717864 |
| Phase-1 RCT-83 | 0.946241 | 1.0609703 | 0.8789166 | 1.1225398 | 0.8822197 | 1.0940223 | 0.72824 |
| Phase-1 RCT-49 | 1.0511899 | 1.0978701 | 1.0819949 | 0.9758607 | 1.0243053 | 1.0191998 | 1.2770728 |
| Phase-1 RCT-48 | 0.8201005 | 0.9948941 | 0.6448996 | 1.1675109 | 1.0938517 | 1.0088116 | 1.3505663 |
| Phase-1 RCT-40 | 1.0655042 | 1.030483 | 0.9564545 | 1.0288253 | 1.0145277 | 0.9058137 | 1 |
| Phase-1 RCT-296 | 0.5165336 | 0.6879827 | 0.8048409 | 0.807766 | 0.3170394 | 0.1576684 | 0.1380627 |
| Phase-1 RCT-295 | 0.9490663 | 1.2591591 | 0.9936857 | 1.0582712 | 1.2622952 | 1.2053946 | 2.2961106 |
| Phase-1 RCT-291 | 0.6635509 | 0.7203682 | 0.7327291 | 1.0384343 | 0.9727216 | 0.8252466 | 0.9585801 |
| Phase-1 RCT-270 | 1.034416 | 0.9710582 | 1.0753901 | 0.8935978 | 0.8142408 | 0.6483021 | 0.4654501 |
| Phase-1 RCT-241 | 1.0029461 | 1.2254524 | 0.9941953 | 1.1735768 | 1.3099661 | 1.2119621 | 1.4434941 |
| Phase-1 RCT-191 | 1.1825393 | 0.8426741 | 1.1506138 | 0.9129049 | 0.9922228 | 0.9284492 | 1.336952 |
| Phase-1 RCT-189 | 1.2262198 | 1.4455162 | 1.2032523 | 1.0133795 | 0.9018568 | 0.8419196 | 0.7943921 |
| Phase-1 RCT-179 | 1.0726161 | 1.2481211 | 1.056878 | 0.9335811 | 1.1462737 | 0.9050572 | 2.0659199 |
| Phase-1 RCT-152 | 1.0164213 | 1.475296 | 1.2105607 | 1.0186201 | 1.3991824 | 1.461649 | 2.4885514 |
| Phase-1 RCT-123 | 0.9876806 | 1.005618 | 1.0056612 | 0.8500373 | 0.9463656 | 0.8279407 | 1.0456605 |
| Paraoxonase 1 | 0.6222873 | 0.6182429 | 0.7779973 | 0.8478296 | 0.731044 | 0.6989097 | 0.6636049 |
| Organic anion transporter 3 | 0.8742976 | 0.6332315 | 0.8260844 | 1.0516382 | 0.9928489 | 1.1492584 | 0.777347 |
| N-hydroxy-2-acetylaminofluorene | 1.3558375 | 0.9544282 | 1.1400536 | 1.0203187 | 0.6867953 | 0.9250276 | 0.3761568 |
| sulfotransferase (ST1C1) | | | | | | | |
| Integrin beta1 | 1.156407 | 1.1001058 | 1.0946438 | 1.1204696 | 1.1972289 | 1.2522796 | 1.582949 |
| Insulin-like growth factor binding protein 3 | 0.7449726 | 0.7520943 | 0.9040868 | 0.9999999 | 0.8656419 | 0.9516107 | 1.3257849 |
| ID-1 | 1.120886 | 0.996605 | 0.9757787 | 1.0296733 | 1.0083055 | 1.1466665 | 1.1317737 |
| Hepatic lipase | 0.633352 | 0.9366249 | 0.8871704 | 0.927583 | 0.7418401 | 0.5393627 | 0.5445957 |
| Heme oxygenase | 0.8179004 | 0.7776252 | 0.6889211 | 1.054812 | 0.842495 | 1.0663974 | 1.0806013 |
| Equilbrative nitrobenzylthioinosine- | 1.0643631 | 0.9689576 | 0.7901226 | 2.2489514 | 0.8645093 | 1.0091199 | 0.7823181 |
| sensitive nucleoside transporter | | | | | | | |
| Epidermal growth factor | 0.7938959 | 1.0199218 | 0.7484019 | 1.0575583 | 0.9148485 | 0.999947 | 0.8101736 |
| c-myc | 1.1600162 | 0.7798084 | 1.3268955 | 1.2134513 | 0.5801836 | 1.0663662 | 0.8349579 |
| Carbonic anhydrase III | 0.6017597 | 0.7164182 | 0.5959066 | 0.5902854 | 0.6998071 | 0.1186656 | 0.3154139 |
| Beta-actin | 1.1000493 | 1.1381011 | 0.8437967 | 0.9697028 | 1.425739 | 0.9416215 | 1.750575 |
| Bax (alpha) | 1.2905521 | 0.9723958 | 1.2563033 | 1.0679818 | 1.332116 | 1.4372963 | 2.6248136 |
| Alpha 1 - inhibitor III | 0.6692346 | 0.6614468 | 0.6952176 | 0.6786019 | 0.8037288 | 0.4356084 | 0.4971736 |
| 60S ribosomal protein L6 | 1.064129 | 1.2614971 | 1.0416728 | 1.0071745 | 1.9609026 | 2.0688612 | 4.097095 |
| Phase-1 RCT-117 | 0.7606593 | 1.1731318 | 0.9031236 | 1.152223 | 1.1572988 | 0.9034138 | 0.9835641 |
| Phase-1 RCT-102 | 0.7510501 | 0.562187 | 0.6684065 | 0.9263886 | 0.5081999 | 0.4625911 | 0.5405393 |
| 3-hydroxyisobutyrate dehydrogenase | 0.8245567 | 1.0574955 | 0.8511097 | 0.8738359 | 1.0029895 | 0.8101313 | 1.0050355 |
| ATPase inhibitor (rat mitochondrial IF1 | 1.1606925 | 1.284846 | 1.0856704 | 0.7636657 | 1.2291912 | 0.7398764 | 1.5151533 |
| protein) | | | | | | | |
| Alpha-2-macroglobulin | 1.1288669 | 0.7983252 | 0.7411193 | 1.0926673 | 0.8258417 | 1.3337142 | 6.123466 |
| Phase-1 RCT-137 | 0.8817021 | 1.2939056 | 1.1053284 | 0.7888222 | 1.2868757 | 0.8316168 | 1.3951693 |
| Phase-1 RCT 252 | 0.8428423 | 1.1304859 | 0.8692793 | 0.7688876 | 1.4754044 | 0.7099103 | 1.6806287 |
| Phase-1 RCT-65 | 1.1157068 | 0.8537874 | 1.134871 | 1.043522 | 1.0860897 | 1.1386333 | 1.3903891 |
| Liver fatty acid binding protein | 0.9869096 | 0.6954176 | 0.9069215 | 0.3963215 | 0.6076953 | 0.5953544 | 0.6394367 |
| Carbamyl phosphate synthetase I | 0.8372406 | 1.1479684 | 0.8697147 | 0.7127037 | 1.4801992 | 0.6817931 | 1.7591617 |
| c-jun | 1.052368 | 0.7383019 | 1.117 | 0.96753 | 0.9888616 | 1.1022645 | 0.7400711 |
| Transthyretin | 0.7787316 | 0.626974 | 0.7831761 | 0.7153317 | 0.5126207 | 0.5141993 | 0.4829779 |
| Aflatoxin B1 aldehyde reductase | 0.9522463 | 0.7044434 | 0.8994162 | 0.803943 | 0.8629971 | 0.7075055 | 0.6874692 |
| Phase-1 RCT-15 | 1.1295125 | 1.0256214 | 1.1426187 | 1.0787915 | 1.0477911 | 1.1213465 | 1.0916415 |
| Insulin-like growth factor I | 0.7347667 | 0.7698456 | 0.9358119 | 0.848608 | 0.7296041 | 0.8255787 | 1.0244377 |
| Sodium/bile acid cotransporter | 0.8794049 | 0.599444 | 0.846965 | 1.0990115 | 1.0279434 | 0.8773332 | 0.6135982 |
| Organic cation transporter 3 | 1.0086356 | 1.2306463 | 1.014775 | 1.044702 | 1.624847 | 1.7855703 | 3.3090835 |
| Gadd153 | 0.9737433 | 1.1167201 | 0.9966002 | 1.0698527 | 1.2216476 | 1.2547026 | 1.7194129 |
| Phase-1 RCT-109 | 1.1508441 | 1.0600932 | 1.1000482 | 0.8540916 | 1.9160272 | 1.8740226 | 0.7387065 |
| Phase-1 RCT-88 | 0.8234285 | 0.8871763 | 1.1379418 | 1.1482127 | 1.0050626 | 0.954527 | 0.7712234 |
| Ubiquitin conjugating enzyme (RAD 6 | 1.0114896 | 1.1651388 | 1.023601 | 0.804633 | 1.3560141 | 1.4869399 | 1.7611756 |
| homologue) | | | | | | | |
| Glucose-regulated protein 78 | 0.9793551 | 1.3507808 | 1.028638 | 1.0035524 | 2.0084684 | 1.5085858 | 2.0283525 |
| Ribosomal protein L13A | 1.3000224 | 1.1285212 | 1.1976901 | 1.2428577 | 2.2039905 | 2.1535406 | 3.2626903 |
| Monocyte chemotactic protein receptor | 1.1019106 | 1.3313829 | 1.2484472 | 1.0345764 | 0.9872893 | 1.109927 | 1.1513957 |
| (CCR2) | | | | | | | |
| Fas antigen | 1.2569524 | 1.2571596 | 1.0056059 | 1.0293813 | 1.1938522 | 1.0163693 | 1.1966382 |
| Calpactin I heavy chain | 1.0945944 | 1.0150394 | 1.0288849 | 1.132874 | 1.072285 | 1.1169147 | 1.2655613 |
| Uncoupling protein 2 | 1.29301 | 1.2453775 | 1.1939926 | 1.1184211 | 1.1337523 | 1.0837348 | 1.3765388 |
| Phase-1 RCT-168 | 0.955419 | 1.1754576 | 0.8905687 | 0.7700366 | 0.6781263 | 0.7513695 | 0.5750061 |
| Phase-1 RCT-154 | 0.9400862 | 1.2462798 | 1.0049834 | 1.0083412 | 2.288147 | 2.383056 | 4.6043005 |
| Superoxide dismutase Mn | 1.1319547 | 2.0200396 | 1.6057551 | 1.2421241 | 1.428933 | 1.1789733 | 2.8132963 |
| Phase-1 RCT-214 | 0.8699653 | 0.9585503 | 0.8533242 | 0.9597128 | 0.6209476 | 0.82784 | 0.7056426 |
| Phase-1 RCT-225 | 0.7760323 | 0.4345704 | 0.7331903 | 0.9199421 | 0.8250576 | 0.8495191 | 0.812194 |
| Phase-1 RCT-181 | 0.8042991 | 0.7799806 | 0.9033365 | 1.1002562 | 1.438414 | 1.3354911 | 1.590165 |
| Nucleoside diphosphate kinase beta | 0.8089485 | 0.9498586 | 0.8216103 | 1.0880276 | 1.4795583 | 1.2451506 | 2.1025286 |
| isoform | | | | | | | |
| Extracellular-signal-regulated kinase 1 | 0.670538 | 0.7788072 | 0.6529602 | 0.6243116 | 0.8044816 | 0.4902078 | 0.6140661 |
| {Ribosomal protein L6} | 0.9921517 | 1.1173406 | 1.0331165 | 0.9983806 | 1.7308453 | 1.8038887 | 3.9719095 |
| Aldehyde dehydrogenase, microsomal | 0.9661763 | 1.0421156 | 0.9389474 | 1.0036554 | 1.2703005 | 1.0435328 | 1.2191534 |
| p53 | 0.9233488 | 1.1121048 | 0.9235815 | 0.9802263 | 1.1633922 | 1.0944346 | 1.3175641 |
| Phase-1 RCT-239 | 1.0626191 | 0.9298008 | 1.1390542 | 0.8709341 | 0.9535451 | 1.0813566 | 1.259676 |
| Macrophage inflammatory protein-2 alpha | 1.1380638 | 0.9401254 | 1.3124572 | 0.9919074 | 1.1185317 | 1.1487525 | 1.4402591 |
| IgE binding protein | 1.1743172 | 1.9268948 | 1.051405 | 1.0970899 | 1.2579374 | 0.9718072 | 1.781124 |
| Phase-1 RCT-205 | 1.117025 | 1.7558326 | 1.0296993 | 0.9056635 | 0.9776244 | 0.9664341 | 0.9356605 |
| Melanoma-associated antigen ME491 | 1.5448022 | 1.232766 | 1.1828952 | 2.1181684 | 1.706025 | 1.0779825 | 2.480578 |
| Phase-1 RCT-242 | 1.2483943 | 1.107811 | 1.4089146 | 0.8617709 | 0.9050089 | 1.0695999 | 0.8146787 |
| Enolase alpha | 1.034378 | 0.9679791 | 0.7521752 | 1.2985603 | 2.2568574 | 2.2174652 | 2.6117344 |
| Thymosin beta-10 | 1.0511589 | 1.2702259 | 0.8051984 | 1.2473649 | 1.6998695 | 1.5861318 | 2.4944253 |
| Cofilin | 0.8631229 | 1.1051084 | 0.9577698 | 1.0442724 | 1.4381027 | 1.2833525 | 1.6438514 |
| Carbonyl reductase | 1.0569657 | 1.0395573 | 1.1370679 | 1.032963 | 0.944806 | 2.4768586 | 0.9190734 |
| MAP kinase kinase | 0.9585434 | 0.9788318 | 0.90606 | 1.2150109 | 1.0318556 | 1.1220206 | 1.2011533 |
| Phase-1 RCT-162 | 1.0025722 | 0.97233 | 1.0056994 | 1.1152316 | 1.1696951 | 1.2903702 | 1.9161958 |
| Phase-1 RCT-72 | 1.0954493 | 1.0639043 | 1.0577831 | 1.0436133 | 1.0338454 | 1.1772991 | 1.7775879 |
| Ribosomal protein S8 | 1.0098487 | 1.0723845 | 1.0577004 | 1.1718295 | 1.884867 | 2.162969 | 3.6621451 |
| Proliferating cell nuclear antigen gene | 1.1566243 | 1.023866 | 1.4357682 | 0.8801625 | 0.6795334 | 1.097543 | 0.9041886 |
| Phase-1 RCT-192 | 0.883417 | 0.88167 | 0.9416277 | 0.7374161 | 0.9454689 | 0.8100978 | 1.3936177 |
| IkB-a | 1.0376899 | 1.081449 | 1.0303317 | 1.1091301 | 1.1397011 | 0.9908223 | 1.3391433 |
| Endogenous retroviral sequence, 5′ and | 1.1356703 | 0.6706346 | 0.9498298 | 0.9027519 | 0.8773599 | 0.9999999 | 1.0143306 |
| 3′ LTR | | | | | | | |
| Phase-1 RCT-37 | 0.975009 | 1.0361996 | 1.0092484 | 1.0827104 | 1.3925552 | 1.3491938 | 1.8995149 |
| Matrix metalloproteinase-1 | 1.0805334 | 1.0915244 | 1.0991398 | 1.1867055 | 1.3227838 | 1.3109212 | 1.8990906 |
| Cyclin G | 1.1416441 | 1.2275662 | 1.2799505 | 0.9875488 | 1.5124706 | 1.2798419 | 3.440135 |
| DNA polymerase beta | 0.9069923 | 1.0130097 | 0.9630096 | 1.0409629 | 1.1214626 | 1.1457049 | 1.4635317 |
| Phase-1 RCT-127 | 0.9801484 | 1.0457485 | 1.1458557 | 0.9068652 | 1.0493762 | 1.0344626 | 1.3987466 |
| Multidrug resistant protein-2 | 1.0635747 | 0.6692764 | 1.2040455 | 1.3219597 | 1.0919524 | 1.3476552 | 3.5141747 |
| Ribosomal protein S9 | 0.9932442 | 1.0393901 | 1.0239768 | 0.944808 | 2.0597034 | 1.9918705 | 2.6471243 |
| Phase-1 RCT-12 | 1.1744244 | 1.0385827 | 1.1618084 | 0.9293912 | 0.9847956 | 0.9065255 | 0.883176 |
| Beta-tubulin, class I | 1.3497293 | 1.0560118 | 1.3553591 | 1.024464 | 1.5757039 | 1.614378 | 2.2946734 |
| Argininosuccinate lyase | 1.1479695 | 1.0996364 | 1.1858492 | 1.0318185 | 1.1400254 | 1.2545941 | 3.0696962 |
| Elongation factor-1 alpha | 0.8436397 | 0.82741 | 0.876581 | 0.9953771 | 1.2205302 | 1.0613021 | 2.113279 |
| Phase-1 RCT-55 | 1.0185466 | 1.0482085 | 1.0882783 | 0.9723486 | 0.9965327 | 1.0868146 | 1.6868674 |
| Macrophage inflammatory protein-1 alpha | 1.1809922 | 1.3437251 | 1.3824635 | 0.9337041 | 0.8573542 | 0.9841824 | 0.8863171 |
| c-H-ras | 1.1547298 | 0.9677838 | 0.9584786 | 1.0713621 | 1.3540628 | 1.4434841 | 2.0440903 |
| Multidrug resistant protein-1 | 1.1038035 | 0.6939005 | 1.2687087 | 1.1624674 | 1.6557432 | 1.9675676 | 5.0458617 |
| T-cell cyclophilin | 0.8570503 | 1.0316645 | 0.9251252 | 1.085256 | 1.2782106 | 1.1775538 | 1.5258083 |
| Phase-1 RCT-185 | 0.8264903 | 0.9322674 | 0.8055021 | 0.9971198 | 0.7111179 | 0.5125071 | 0.5316933 |
| 25-DX | 0.9372149 | 0.7882471 | 0.8862107 | 0.8427508 | 1.106385 | 0.6038504 | 0.9266605 |
| Ref-1 | 1.2169021 | 1.2365209 | 1.1433469 | 1.1780521 | 1.0908308 | 1.2381445 | 1.262897 |
| Apolipoprotein CIII | 0.8765931 | 0.9606628 | 0.8188712 | 0.829578 | 0.6892585 | 0.5908861 | 0.5461198 |
| NADPH quinone oxidoreductase-1 (DT- | 1.5874805 | 1.6113678 | 1.2255836 | 1.9117788 | 1.2035342 | 1.2773225 | 1.0065181 |
| diaphorase) | | | | | | | |
| Pyruvate kinase, muscle | 0.9015758 | 0.9270653 | 0.9446133 | 1.2178328 | 1.2472234 | 1.2232014 | 1.8739325 |
| | CHEX | CHEX | CPHOS | CPHOS | CPHOS | CPHOS | CPHOS |
| Compound-Dose (2) | 2 | 2 | 25 | 25 | 25 | 100 | 100 |
| Animal Number (3) | 2255 | 2256 | 2144 | 2145 | 2146 | 2154 | 2155 |
| Liver Toxicity Necrosis Classification (4) | no | no | no | no | no | no | no |
| Gene Name (5) | | | | | | | |
| Phase-1 RCT-78 | 0.667798 | 0.6463164 | 0.9391316 | 0.886846 | 0.9805226 | 0.6707907 | 0.8855752 |
| Gamma-actin, cytoplasmic | 2.8307903 | 2.7995775 | 1.7562884 | 0.9431759 | 1.1469854 | 1.729922 | 0.9863684 |
| Matrin F/G | 0.6684219 | 0.8266303 | 1.1916116 | 1.2122637 | 0.8718199 | 1.3490194 | 1.1634636 |
| Zinc finger protein | 2.5314925 | 1.0313812 | 0.9909809 | 1.0342678 | 1.0761296 | 1.0043882 | 1.1008052 |
| Cathepsin L, sequence 2 | 3.2933927 | 1.7515085 | 1.0889438 | 1.1396464 | 0.7879411 | 2.5087938 | 0.9960531 |
| Gadd45 | 1.0677212 | 0.9225125 | 0.9510075 | 0.9302086 | 1.2260978 | 0.8098992 | 0.9251846 |
| Phase-1 RCT-144 | 1.6981608 | 1.5520238 | 0.9566223 | 0.9453365 | 1.2069634 | 0.8011884 | 0.827708 |
| Phase-1 RCT-145 | 1.9621283 | 1.5309699 | 0.9329095 | 0.9407846 | 1.1930208 | 0.9485446 | 0.972011 |
| Phase-1 RCT-50 | 3.394043 | 1.8673415 | 1.034398 | 1.1886325 | 1.0005766 | 0.9330783 | 0.9171667 |
| Phase-1 RCT-92 | 0.5483356 | 0.623581 | 0.7802805 | 0.7807231 | 0.7512972 | 0.7781508 | 1.1181778 |
| Phase-1 RCT-89 | 0.391246 | 0.6660112 | 0.9264467 | 1.0080161 | 0.8210696 | 0.8326018 | 1.0771154 |
| 14-3-3 zeta | 1.307878 | 1.21656 | 1.1839417 | 1.1401983 | 0.9397132 | 1.3926954 | 1.0973746 |
| Dynamin-1 (D100) | 0.7964142 | 0.6968291 | 0.9778819 | 1.1594439 | 1.0533482 | 0.8876348 | 0.998888 |
| Insulin-like growth factor binding protein 1 | 1.8908749 | 1.0158372 | 1.1673372 | 1.3398731 | 0.9290115 | 2.7482727 | 0.9617714 |
| L-gulono-gamma-lactone oxidase | 0.1814972 | 0.3251369 | 0.9031416 | 1.1519494 | 0.7240199 | 0.7478321 | 0.9085025 |
| Ornithine decarboxylase | 1.8088492 | 2.528514 | 1.176092 | 1.1380397 | 1.2392683 | 1.6271954 | 1.2315495 |
| PAR interacting protein | 2.4942105 | 1.7751994 | 0.9853479 | 0.8818699 | 1.0005305 | 0.9074348 | 0.850068 |
| Phase-1 RCT-128 | 0.7357793 | 0.5600781 | 0.9568636 | 1.2518427 | 0.7223218 | 1.1631421 | 1.0494541 |
| Phase-1 RCT-180 | 1.9922869 | 1.7307063 | 1.0763104 | 0.9311662 | 0.9008316 | 0.9629032 | 1.342655 |
| Phase-1 RCT-182 | 0.6479557 | 0.4738569 | 0.8137107 | 0.7286113 | 0.6249178 | 0.7487153 | 1.1040483 |
| Phase-1 RCT-207 | 3.5395994 | 2.2061093 | 0.9999135 | 1.1275882 | 1.1160359 | 1.4032772 | 0.9619898 |
| Phase-1 RCT-213 | 2.2978952 | 1.5577714 | 0.9194017 | 0.931604 | 1.1387705 | 1.3266455 | 1.0444399 |
| Phase-1 RCT-256 | 0.747829 | 1.31074 | 1.0140744 | 1.1056498 | 0.6972737 | 0.9440794 | 1.1170622 |
| Phase-1 RCT-258 | 1.3913066 | 1.2058332 | 0.9523556 | 0.9593832 | 1.2935637 | 1.0043358 | 1.0006169 |
| Phase-1 RCT-264 | 0.325804 | 0.2879862 | 1.0200475 | 1.0137259 | 0.8744989 | 0.7874297 | 1.3133297 |
| Phase-1 RCT-271 | 0.6862688 | 0.7350636 | 0.8512117 | 0.9533653 | 0.7467103 | 0.6419573 | 0.7477193 |
| Phase-1 RCT-288 | 0.7065826 | 0.6813062 | 0.9689823 | 1.0845494 | 0.8318668 | 0.9535464 | 1.2355851 |
| Phase-1 RCT-38 | 0.8074279 | 1.4364172 | 1.0438038 | 1.076891 | 0.6634227 | 0.9654182 | 1.1403881 |
| Phase-1 RCT-39 | 1.309749 | 1.4161114 | 1.0445861 | 1.0353231 | 0.9387819 | 1.1017482 | 0.8511754 |
| Phase-1 RCT-68 | 1.3639582 | 1.1215438 | 1.0802022 | 1.062245 | 1.2459819 | 1.5620099 | 1.0761513 |
| Phase-1 RCT-33 | 1.1201547 | 1.1286113 | 1.0311242 | 1.03702 | 0.9894077 | 0.8278367 | 1.0451841 |
| Phase-1 RCT-36 | 0.9522938 | 0.9535317 | 1.0611111 | 0.9877064 | 1.026631 | 0.8405287 | 1.019746 |
| 17-beta hydroxysteroid dehydrogenase, | 0.8086311 | 0.8288592 | 0.4939154 | 1.3243837 | 0.8245766 | 0.2018843 | 0.5603041 |
| type 2 | | | | | | | |
| Senescence marker protein-30 | 0.085319 | 0.454931 | 1.1123818 | 1.0450069 | 0.6272208 | 0.2785304 | 1.4121476 |
| Ribosomal protein S17 | 3.700771 | 3.3105054 | 0.972983 | 0.9247206 | 0.888338 | 1.409621 | 1.0901618 |
| Phase-1 RCT-83 | 0.7709396 | 0.5883393 | 1.5859932 | 0.8287824 | 1.0944492 | 0.6857336 | 1.0074972 |
| Phase-1 RCT-49 | 2.2791853 | 1.1162382 | 0.9349288 | 0.9993536 | 1.2135141 | 0.9428837 | 0.8715959 |
| Phase-1 RCT-48 | 0.8259009 | 0.9012404 | 0.9687007 | 1.1085708 | 0.8232026 | 0.7872354 | 1.011728 |
| Phase-1 RCT-40 | 0.5867795 | 0.6252523 | 0.9869798 | 0.9476073 | 0.7827327 | 0.6626859 | 0.9190771 |
| Phase-1 RCT-296 | 0.1324876 | 0.1028095 | 1.01345 | 0.7855847 | 0.6128551 | 0.158913 | 0.8048253 |
| Phase-1 RCT-295 | 1.8094178 | 1.772306 | 1.1690981 | 1.2003773 | 0.7264906 | 1.4765191 | 1.0772991 |
| Phase-1 RCT-291 | 0.9117413 | 0.8283461 | 0.9644906 | 0.9999749 | 0.8076509 | 0.9278626 | 1.0587115 |
| Phase-1 RCT-270 | 0.3824071 | 0.3778883 | 0.9663873 | 0.8189346 | 0.6831677 | 0.7237883 | 1.2349594 |
| Phase-1 RCT-241 | 2.8054178 | 1.6259654 | 0.888283 | 0.8516127 | 1.2378597 | 0.9822884 | 1.0866003 |
| Phase-1 RCT-191 | 1.4646564 | 1.3259895 | 0.9705969 | 1.0260553 | 1.3154 | 1.0503707 | 1.0233644 |
| Phase-1 RCT-189 | 0.7556233 | 0.7412518 | 0.9615324 | 1.0974708 | 0.707485 | 0.9764776 | 0.8922575 |
| Phase-1 RCT-179 | 1.8741211 | 1.7257137 | 0.9472554 | 0.9971207 | 0.7794477 | 1.032192 | 0.8451706 |
| Phase-1 RCT-152 | 3.0684192 | 2.1494024 | 0.9267474 | 0.8689169 | 1.087118 | 1.1847246 | 0.8551658 |
| Phase-1 RCT-123 | 0.87194 | 0.9461917 | 0.8935726 | 0.8496512 | 1.1602521 | 0.9244103 | 0.9581271 |
| Paraoxonase 1 | 0.4606977 | 0.4822132 | 0.9000111 | 0.7703277 | 0.7320197 | 0.7502915 | 1.3107543 |
| Organic anion transporter 3 | 0.6196384 | 0.8225944 | 1.1989499 | 1.2403215 | 0.844389 | 1.1526127 | 0.9666416 |
| N-hydroxy-2-acetylaminofluorene | 0.1069238 | 0.239772 | 0.7895198 | 0.77608 | 0.567018 | 0.2502532 | 0.8187832 |
| sulfotransferase (ST1C1) | | | | | | | |
| Integrin beta1 | 1.9727416 | 1.5494436 | 1.1614805 | 1.1736794 | 0.967523 | 1.665677 | 0.9991933 |
| Insulin-like growth factor binding protein 3 | 0.6014795 | 0.8706371 | 1.2620434 | 1.3743546 | 0.9277635 | 0.4986485 | 0.9784009 |
| ID-1 | 1.8312454 | 1.4756345 | 1.4420907 | 1.1872588 | 1.5162722 | 0.9753945 | 1.353634 |
| Hepatic lipase | 0.4250339 | 0.4085319 | 0.9466535 | 0.9833831 | 0.6945092 | 0.4661904 | 0.7755381 |
| Heme oxygenase | 2.502657 | 1.1108916 | 0.9534873 | 0.8807262 | 1.1531132 | 0.8352884 | 0.9393536 |
| Equilbrative nitrobenzylthioinosine- | 0.4340071 | 0.4974363 | 0.839089 | 0.8169304 | 0.7425929 | 0.4522161 | 0.8570891 |
| sensitive nucleoside transporter | | | | | | | |
| Epidermal growth factor | 0.7851437 | 0.8390522 | 1.0683367 | 1.0456637 | 0.8550362 | 1.0460306 | 1.0301778 |
| c-myc | 1.6828226 | 1.1433551 | 0.9533054 | 0.9119935 | 1.6357614 | 0.896512 | 0.9532857 |
| Carbonic anhydrase III | 0.0981724 | 0.1170097 | 0.9088653 | 1.0083947 | 0.682052 | 0.4819486 | 0.9202982 |
| Beta-actin | 1.265545 | 1.5824912 | 2.3040056 | 2.0273225 | 1.07393 | 2.7878108 | 1.2672379 |
| Bax (alpha) | 3.9086614 | 3.0786192 | 1.2386264 | 1.2369996 | 1.3569362 | 1.2693458 | 0.933848 |
| Alpha 1 - inhibitor III | 0.1984895 | 0.3033326 | 0.8217765 | 1.0460413 | 0.6627238 | 0.4861853 | 0.7626263 |
| 60S ribosomal protein L6 | 4.045918 | 3.1642585 | 0.9039314 | 0.90144 | 0.8838301 | 1.149436 | 1.0282967 |
| Phase-1 RCT-117 | 0.5435337 | 0.8113733 | 1.0592916 | 1.153077 | 0.8979352 | 0.8736454 | 1.0590727 |
| Phase-1 RCT-102 | 0.1629469 | 0.3218882 | 0.856752 | 0.8379034 | 1.2560108 | 0.1936154 | 0.5372141 |
| 3-hydroxyisobutyrate dehydrogenase | 0.7821577 | 0.6853831 | 0.9804944 | 1.049768 | 0.7488565 | 0.9559243 | 1.0686826 |
| ATPase inhibitor (rat mitochondrial IF1 | 1.1272709 | 1.103547 | 0.7509297 | 0.9233325 | 0.8846574 | 0.6961141 | 0.9379382 |
| protein) | | | | | | | |
| Alpha-2-macroglobulin | 8.537437 | 10.8864 | 0.8460496 | 0.9185173 | 0.7109078 | 1.376476 | 0.7513375 |
| Phase-1 RCT-137 | 0.9967647 | 0.8970311 | 0.7713414 | 0.9292721 | 0.7513132 | 0.759066 | 1.1532505 |
| Phase-1 RCT 252 | 2.1088016 | 1.3155844 | 0.7723637 | 1.134687 | 0.6007874 | 1.8449084 | 1.3955609 |
| Phase-1 RCT-65 | 1.0703056 | 1.2085007 | 1.1739428 | 1.1806263 | 1.3234937 | 1.6624568 | 1.1367786 |
| Liver fatty acid binding protein | 0.1219566 | 0.3543471 | 1.4778444 | 1.0908494 | 0.7643772 | 0.3482527 | 0.9220613 |
| Carbamyl phosphate synthetase I | 2.338573 | 1.4254993 | 0.776629 | 1.1883606 | 0.5561633 | 1.9909643 | 1.476023 |
| c-jun | 1.5913497 | 1.1105604 | 1.1344666 | 1.0432019 | 1.5495086 | 0.9971519 | 0.7638058 |
| Transthyretin | 0.3340674 | 0.2878656 | 0.9173054 | 0.9384143 | 0.8358054 | 0.6669698 | 1.0750316 |
| Aflatoxin B1 aldehyde reductase | 0.3081197 | 0.5568926 | 0.6742587 | 0.8572652 | 0.9458898 | 0.6588352 | 0.7015384 |
| Phase-1 RCT-15 | 2.0978093 | 1.2427815 | 1.1823878 | 1.0841051 | 1.2732364 | 1.5478154 | 1.1188401 |
| Insulin-like growth factor I | 0.6320406 | 0.7238303 | 1.2214048 | 1.2864136 | 0.7688987 | 0.4316746 | 0.9099649 |
| Sodium/bile acid cotransporter | 0.2803164 | 0.3732801 | 1.0687011 | 1.0116426 | 0.7821948 | 0.4844858 | 0.9022802 |
| Organic cation transporter 3 | 3.2877293 | 2.7987916 | 0.936944 | 0.9030965 | 0.9971304 | 1.0487641 | 1.0399148 |
| Gadd153 | 2.3482122 | 2.1793523 | 1.107138 | 1.0884286 | 1.2165945 | 1.2304094 | 1.0477052 |
| Phase-1 RCT-109 | 0.8440098 | 0.9526926 | 1.3006603 | 1.4978039 | 0.905875 | 1.4498234 | 0.8706095 |
| Phase-1 RCT-88 | 0.5622494 | 0.6990299 | 0.7544847 | 0.7727982 | 0.8997526 | 0.9204478 | 0.9590114 |
| Ubiquitin conjugating enzyme (RAD 6 | 1.7884897 | 1.6240944 | 0.9811337 | 1.0571078 | 0.8291071 | 1.3405486 | 1.0407757 |
| homologue) | | | | | | | |
| Glucose-regulated protein 78 | 1.3342218 | 1.9431802 | 1.3781518 | 1.2836183 | 0.5750642 | 1.6278014 | 1.3265496 |
| Ribosomal protein L13A | 3.2222893 | 2.5970266 | 1.4579102 | 1.6483915 | 0.8601038 | 1.6183573 | 0.8677086 |
| Monocyte chemotactic protein receptor | 1.1846683 | 1.3560402 | 0.9134791 | 0.9293504 | 1.7072501 | 0.9161365 | 1.0207207 |
| (CCR2) | | | | | | | |
| Fas antigen | 1.6553521 | 1.5438404 | 1.2888573 | 1.406916 | 0.9112303 | 1.6140989 | 1.140782 |
| Calpactin I heavy chain | 1.2104383 | 1.277796 | 1.2291756 | 1.2400712 | 1.2377505 | 1.0774096 | 1.0660698 |
| Uncoupling protein 2 | 1.418773 | 1.4214371 | 1.0358721 | 1.0957901 | 1.4018011 | 0.9256125 | 0.777016 |
| Phase-1 RCT-168 | 0.2586505 | 0.400393 | 1.0856777 | 0.9271205 | 0.8776507 | 0.8368063 | 1.1428528 |
| Phase-1 RCT-154 | 4.404344 | 4.1387997 | 0.9700533 | 0.9576501 | 1.115014 | 1.1224256 | 1.0526112 |
| Superoxide dismutase Mn | 3.631148 | 2.358562 | 1.1644005 | 1.1525052 | 0.8381789 | 1.1752942 | 0.9713565 |
| Phase-1 RCT-214 | 0.2815282 | 0.4923604 | 1.1638097 | 1.0658029 | 0.7476404 | 0.7964751 | 1.4353431 |
| Phase-1 RCT-225 | 0.8232756 | 0.5193017 | 0.9201934 | 0.8514112 | 0.7569802 | 1.3456092 | 1.2263802 |
| Phase-1 RCT-181 | 1.0510261 | 1.1358595 | 0.9277747 | 1.0243663 | 0.9455357 | 0.9942644 | 1.1455501 |
| Nucleoside diphosphate kinase beta | 1.9090911 | 1.7552234 | 1.1120044 | 1.0174289 | 0.9342405 | 1.6182374 | 1.2571884 |
| isoform | | | | | | | |
| Extracellular-signal-regulated kinase 1 | 0.5344649 | 0.5280631 | 0.9109168 | 0.9754714 | 0.7667931 | 0.5552625 | 0.913246 |
| {Ribosomal protein L6} | 3.7913826 | 2.8316884 | 0.9323775 | 1.0229768 | 0.8493095 | 1.2114961 | 0.9543652 |
| Aldehyde dehydrogenase, microsomal | 0.9856865 | 0.9884456 | 1.0687747 | 1.1520454 | 0.8742379 | 0.8473994 | 0.9386944 |
| p53 | 1.8704671 | 1.6200306 | 0.9119639 | 0.906875 | 0.9641649 | 0.9555383 | 0.8967571 |
| Phase-1 RCT-239 | 1.1736157 | 1.6064131 | 1.1130126 | 1.1102828 | 1.3730361 | 0.8380306 | 0.9768531 |
| Macrophage inflammatory protein-2 alpha | 2.1188757 | 2.1920779 | 1.0363132 | 0.9630271 | 1.2813107 | 0.9745143 | 0.9293999 |
| IgE binding protein | 2.930634 | 1.5511414 | 1.0512024 | 0.9398536 | 1.3695757 | 0.966367 | 0.9916483 |
| Phase-1 RCT-205 | 1.4146328 | 0.8011008 | 0.9376333 | 0.9175087 | 1.1424867 | 0.8525526 | 0.9230769 |
| Melanoma-associated antigen ME491 | 1.9088544 | 1.8986208 | 0.994103 | 0.9981464 | 1.09723 | 1.251004 | 0.9720487 |
| Phase-1 RCT-242 | 1.1940968 | 1.0014788 | 1.1034844 | 1.0132798 | 1.6644111 | 1.0566189 | 0.9509832 |
| Enolase alpha | 1.8820095 | 1.7663839 | 1.1835247 | 0.9912332 | 0.7832362 | 2.0236657 | 1.3261403 |
| Thymosin beta-10 | 2.0289629 | 2.0181258 | 1.3100605 | 1.4969864 | 0.8640092 | 1.3471377 | 0.8864974 |
| Cofilin | 1.457137 | 1.3132658 | 0.9478236 | 0.8675892 | 0.8927991 | 1.077763 | 1.1043705 |
| Carbonyl reductase | 1.1011152 | 1.1157993 | 0.9447666 | 0.8836669 | 1.4660344 | 0.9953131 | 0.946986 |
| MAP kinase kinase | 1.389296 | 1.2024299 | 1.053685 | 1.0350316 | 0.935824 | 0.8967357 | 1.1037039 |
| Phase-1 RCT-162 | 1.5743201 | 1.3396431 | 1.0119911 | 0.9445357 | 0.9827871 | 1.0226735 | 1.0935352 |
| Phase-1 RCT-72 | 1.6667311 | 1.522107 | 0.9206672 | 0.9077973 | 1.2706705 | 0.9313363 | 0.9537962 |
| Ribosomal protein S8 | 3.3911521 | 2.9236145 | 0.9899689 | 0.9037337 | 0.7461551 | 1.1861774 | 1.0670441 |
| Proliferating cell nuclear antigen gene | 1.043779 | 0.9893094 | 0.966058 | 0.9334428 | 1.1885968 | 0.9684168 | 0.9066035 |
| Phase-1 RCT-192 | 1.6649636 | 0.9838191 | 1.0390985 | 1.0116512 | 0.8805418 | 1.2401581 | 1.2396026 |
| IkB-a | 1.2742853 | 0.9567115 | 0.9545653 | 0.9796697 | 0.9446681 | 1.3525469 | 0.9688512 |
| Endogenous retroviral sequence, 5′ and | 0.9066097 | 0.7128742 | 1.213356 | 1.2235526 | 0.8279691 | 2.3012395 | 0.8011342 |
| 3′ LTR | | | | | | | |
| Phase-1 RCT-37 | 1.4997816 | 1.5984524 | 0.9802339 | 0.9762653 | 0.9459132 | 1.1364017 | 1.0073923 |
| Matrix metalloproteinase-1 | 3.1198218 | 2.247403 | 1.3326219 | 1.3320858 | 0.8850892 | 1.3429927 | 0.9671366 |
| Cyclin G | 8.628878 | 4.770808 | 1.2665889 | 1.2254753 | 1.5915552 | 1.2039192 | 1.1884351 |
| DNA polymerase beta | 1.3669251 | 1.2760171 | 1.0109853 | 0.9297993 | 0.8683913 | 1.0195237 | 1.1464632 |
| Phase-1 RCT-127 | 1.6703781 | 1.3068986 | 0.970587 | 0.9767633 | 1.2734109 | 1.034659 | 0.9705572 |
| Multidrug resistant protein-2 | 3.4177349 | 3.1648946 | 1.0715761 | 1.0003902 | 0.9202215 | 0.9192066 | 0.9184761 |
| Ribosomal protein S9 | 1.9152645 | 2.0636494 | 1.0059747 | 0.9552453 | 0.7100436 | 1.3048372 | 1.1824393 |
| Phase-1 RCT-12 | 0.9742653 | 0.7860527 | 1.1598688 | 1.0943879 | 0.9641975 | 1.1685371 | 0.9177635 |
| Beta-tubulin, class I | 1.4241686 | 1.2904816 | 1.1570295 | 1.0440573 | 0.8936613 | 1.1601248 | 1.1619031 |
| Argininosuccinate lyase | 5.4212594 | 2.3782387 | 1.1350992 | 1.277719 | 0.9853934 | 3.3298848 | 1.3016456 |
| Elongation factor-1 alpha | 2.1220472 | 1.6371182 | 0.9851635 | 1.0433565 | 0.8510042 | 1.3022339 | 1.2128466 |
| Phase-1 RCT-55 | 1.3801666 | 1.598299 | 1.1452774 | 0.97103 | 1.4097662 | 1.0168848 | 1.0464588 |
| Macrophage inflammatory protein-1 alpha | 0.9230972 | 1.0700648 | 0.9369015 | 0.9064627 | 1.7060608 | 0.8286141 | 0.9284511 |
| c-H-ras | 1.8799273 | 1.6705265 | 1.1150687 | 1.1782086 | 1.0584335 | 1.384305 | 1.0144197 |
| Multidrug resistant protein-1 | 7.384141 | 7.3518043 | 1.1213814 | 1.0503424 | 0.9900291 | 0.995581 | 0.977274 |
| T-cell cyclophilin | 1.3614658 | 1.1560038 | 0.9775799 | 0.9106141 | 0.7892498 | 1.0598682 | 1.1782238 |
| Phase-1 RCT-185 | 0.3822102 | 0.3980739 | 0.7973408 | 0.8463898 | 0.7658559 | 0.6685737 | 0.8749062 |
| 25-DX | 0.5823775 | 0.7346732 | 0.8957489 | 1.0446537 | 0.7730401 | 1.0096067 | 0.7517986 |
| Ref-1 | 1.5383164 | 1.3712323 | 1.0549197 | 0.9726511 | 1.0595975 | 1.1531125 | 1.0285712 |
| Apolipoprotein CIII | 0.5461478 | 0.502718 | 0.891026 | 0.9310297 | 0.8529257 | 0.8173271 | 0.8642668 |
| NADPH quinone oxidoreductase-1 (DT- | 0.8532323 | 0.8480012 | 1.187104 | 1.1019137 | 1.1113386 | 1.5559 | 1.2621051 |
| diaphorase) | | | | | | | |
| Pyruvate kinase, muscle | 2.1357522 | 1.6510645 | 0.9407241 | 1.0512626 | 1.0550194 | 0.9875792 | 0.6904371 |

(1) Gene expression data for 24 hour timpoint are presented as mean ratio of treatment/control for all 24 hour predictive genes (Table 5).
(2) Compound and dose abbreviations as in Table 1.
(3) Individual animal number
(4) Liver necrosis classification for compound-dose group at 72 h: yes, necrosis observed; no, no necrosis observed
(5) Predictive gene (as in Table 5 and as included in Table 32)

TABLE 35
Expression Data for 24 Hour Timepoint (1)

| | CPHOS | CYCA | CYCA | CYCA | CYCA | CYCA | CYCA |
|---|---|---|---|---|---|---|---|
| Compound-Dose (2) | 100 | 20 | 20 | 20 | 80 | 80 | 80 |
| Animal Number (3) | 2156 | 424 | 425 | 426 | 434 | 435 | 436 |
| Liver Toxicity Necrosis Classification (4) | no | no | no | no | no | no | no |
| Gene Name (5) | | | | | | | |
| Phase-1 RCT-78 | 0.9484483 | 0.9794971 | 1.3069156 | 1.167088 | 0.9789183 | 1.1266862 | 1.0271409 |
| Gamma-actin, cytoplasmic | 1.3634926 | 1.0125684 | 1.0745239 | 1.0903769 | 0.9612417 | 1.3817676 | 0.9044261 |
| Matrin F/G | 0.9591115 | 1.0260894 | 1.2102444 | 1.2889785 | 1.1718001 | 1.2187382 | 1.4787639 |
| Zinc finger protein | 1.1068206 | 0.8486365 | 0.8013943 | 0.967137 | 0.9269166 | 0.9467515 | 1.008149 |
| Cathepsin L, sequence 2 | 1.1702825 | 1.1768477 | 0.9187442 | 1.053295 | 1.325772 | 1.6699927 | 1.2260942 |
| Gadd45 | 0.7904642 | 1.0456836 | 0.898972 | 0.9870908 | 1.1308373 | 1.6655682 | 1.1475778 |
| Phase-1 RCT-144 | 0.9685715 | 0.6244965 | 0.9665857 | 0.8624127 | 0.8728544 | 1.0502961 | 0.8548957 |
| Phase-1 RCT-145 | 1.0006781 | 0.7794119 | 0.8580293 | 0.8403946 | 0.9526797 | 1.0197982 | 0.9157196 |
| Phase-1 RCT-50 | 0.9663577 | 0.8396751 | 0.9605634 | 0.8938182 | 1.0644314 | 0.9487388 | 0.9041121 |
| Phase-1 RCT-92 | 0.870823 | 0.9759136 | 0.9620701 | 0.8869293 | 0.8174908 | 0.7296586 | 0.7213025 |
| Phase-1 RCT-89 | 0.8911229 | 1.1618327 | 1.0946219 | 0.8981101 | 0.7978234 | 0.9068809 | 0.932035 |
| 14-3-3 zeta | 1.0937468 | 1.253323 | 1.0906231 | 1.1158351 | 0.9816425 | 1.1151539 | 1.0103863 |
| Dynamin-1 (D100) | 0.9409692 | 0.8652225 | 1.1071768 | 1.0250887 | 0.9364979 | 0.832732 | 0.9573133 |
| Insulin-like growth factor binding protein 1 | 1.1548257 | 1.2828089 | 1.408841 | 1.4273657 | 1.1655009 | 1.1625943 | 1.3610075 |
| L-gulono-gamma-lactone oxidase | 0.7459693 | 0.8787201 | 0.6397069 | 0.7580304 | 0.670311 | 0.6397245 | 0.712658 |
| Ornithine decarboxylase | 1.2927084 | 0.941588 | 0.8631294 | 1.0292195 | 0.8723652 | 0.9949076 | 0.8576096 |
| PAR interacting protein | 0.9499066 | 0.9062479 | 1.0354124 | 0.9345446 | 0.9905648 | 1.0633708 | 0.8708044 |
| Phase-1 RCT-128 | 0.8783803 | 1.3684152 | 1.3334267 | 1.0783281 | 0.8618171 | 0.7879093 | 0.9581407 |
| Phase-1 RCT-180 | 1.1933385 | 1.0168844 | 0.9215276 | 1.100195 | 1.0865346 | 1.0267407 | 1.2216153 |
| Phase-1 RCT-182 | 0.9999412 | 1.2700888 | 1.2598532 | 0.9704698 | 0.8661587 | 1.0447692 | 0.9196985 |
| Phase-1 RCT-207 | 1.1437858 | 0.8366748 | 1.0595306 | 0.8409313 | 1.1121846 | 0.9441503 | 0.9534003 |
| Phase-1 RCT-213 | 1.2641296 | 0.9356715 | 1.0046576 | 1.1963956 | 1.2202446 | 1.2503719 | 1.2359508 |
| Phase-1 RCT-256 | 0.7931778 | 1.0291848 | 1.0287266 | 1.0267929 | 0.8192412 | 0.8530713 | 0.9592804 |
| Phase-1 RCT-258 | 1.1131915 | 0.8311171 | 0.902467 | 0.8789593 | 0.9955706 | 0.9703758 | 0.9809441 |
| Phase-1 RCT-264 | 0.7448648 | 1.0363969 | 1.2332034 | 0.7722422 | 0.4160352 | 0.5033242 | 0.6194832 |
| Phase-1 RCT-271 | 0.6939642 | 0.8282959 | 0.8386124 | 0.922964 | 0.7056658 | 0.4966346 | 0.7670422 |
| Phase-1 RCT-288 | 1.0077139 | 1.2757496 | 1.3390166 | 1.198772 | 0.7762905 | 0.9089493 | 1.0402434 |
| Phase-1 RCT-38 | 0.7883769 | 0.75825 | 0.9110029 | 0.9498691 | 0.7813118 | 0.8573137 | 0.9862575 |
| Phase-1 RCT-39 | 0.9938932 | 0.9053082 | 0.8490759 | 0.9213972 | 1.1300805 | 1.1195198 | 1.0796058 |
| Phase-1 RCT-68 | 1.0606742 | 0.8869659 | 1.0785478 | 1.0822141 | 1.0803603 | 1.2140108 | 1.1720673 |
| Phase-1 RCT-33 | 0.8107873 | 1.1899189 | 0.943084 | 1.3082712 | 0.8549936 | 0.9939307 | 1.212808 |
| Phase-1 RCT-36 | 0.941996 | 0.8195126 | 0.9264725 | 1.1017169 | 0.9258663 | 0.9367098 | 1.0599449 |
| 17-beta hydroxysteroid dehydrogenase, | 0.4556468 | 0.7315514 | 0.6742402 | 0.6945379 | 0.6129316 | 0.5960956 | 0.7412709 |
| type 2 | | | | | | | |
| Senescence marker protein-30 | 0.7754318 | 1.5519705 | 1.1176964 | 0.9005886 | 0.680923 | 0.6797705 | 1.114304 |
| Ribosomal protein S17 | 1.1473886 | 1.2796603 | 0.9923449 | 1.0618263 | 0.9263767 | 1.3398372 | 1.2199057 |
| Phase-1 RCT-83 | 0.8979311 | 0.7972006 | 0.9840882 | 0.9287447 | 0.7615814 | 0.8230061 | 0.8780805 |
| Phase-1 RCT-49 | 0.9460527 | 0.8171284 | 0.9188861 | 0.9228668 | 0.8637526 | 0.9955895 | 0.9908863 |
| Phase-1 RCT-48 | 0.9022799 | 0.983695 | 1.3176322 | 1.0085266 | 0.8498268 | 0.7513785 | 0.8254458 |
| Phase-1 RCT-40 | 0.9196776 | 1.3069608 | 1.1720355 | 0.9759463 | 0.6401215 | 0.8725444 | 0.8782629 |
| Phase-1 RCT-296 | 0.6015903 | 0.763848 | 0.7476022 | 0.4766074 | 0.2583476 | 0.2967772 | 0.2731636 |
| Phase-1 RCT-295 | 1.0489185 | 1.077714 | 1.0427506 | 1.0050138 | 0.847999 | 1.0633425 | 0.9588534 |
| Phase-1 RCT-291 | 0.845613 | 1.0258198 | 0.9332075 | 0.895806 | 0.9139153 | 0.9513171 | 0.9427471 |
| Phase-1 RCT-270 | 0.9316339 | 1.1744232 | 1.3081051 | 0.9844962 | 0.9266729 | 0.8420352 | 1.0507302 |
| Phase-1 RCT-241 | 1.0945207 | 0.954865 | 0.7859155 | 1.4552926 | 1.3767824 | 2.2799394 | 2.1868176 |
| Phase-1 RCT-191 | 1.0512861 | 0.8547019 | 0.819453 | 0.9079883 | 0.8570923 | 0.9010302 | 0.8206368 |
| Phase-1 RCT-189 | 0.9970363 | 1.2756166 | 1.1144452 | 1.2307031 | 1.0198687 | 1.0475286 | 1.213863 |
| Phase-1 RCT-179 | 0.9044926 | 1.1046842 | 1.0298331 | 0.9401223 | 0.790566 | 0.9349622 | 0.8429226 |
| Phase-1 RCT-152 | 1.0164545 | 1.0511379 | 0.9930704 | 1.0813404 | 1.0562707 | 1.4261972 | 1.0665971 |
| Phase-1 RCT-123 | 0.9684989 | 0.8492146 | 0.9458128 | 0.9414672 | 1.1219648 | 0.9934461 | 1.0085897 |
| Paraoxonase 1 | 0.8712983 | 1.1172482 | 0.9117747 | 0.7901064 | 0.595261 | 0.6978524 | 0.7338647 |
| Organic anion transporter 3 | 0.9397717 | 1.0870864 | 0.9476457 | 0.9168134 | 1.076507 | 0.9376372 | 0.879915 |
| N-hydroxy-2-acetylaminofluorene | 0.5332261 | 0.9321487 | 1.0513389 | 0.8952602 | 0.6630721 | 0.7701613 | 0.5906033 |
| sulfotransferase (ST1C1) | | | | | | | |
| Integrin beta1 | 1.0838394 | 1.0403911 | 1.0899867 | 1.1697568 | 1.207472 | 1.1117179 | 1.0817413 |
| Insulin-like growth factor binding protein 3 | 0.7368286 | 1.0357935 | 1.0737177 | 0.9467605 | 0.6671923 | 0.8182897 | 0.7782388 |
| ID-1 | 1.2512331 | 1.085807 | 1.0849609 | 1.0751164 | 0.8859658 | 1.0061654 | 0.9649399 |
| Hepatic lipase | 0.6520101 | 0.9184179 | 0.8205795 | 0.924792 | 0.6543936 | 0.621842 | 0.690928 |
| Heme oxygenase | 1.030465 | 0.8380553 | 1.01958 | 1.138625 | 1.0007648 | 1.2854139 | 1.425625 |
| Equilbrative nitrobenzylthioinosine- | 0.6998117 | 0.8180151 | 0.9244396 | 0.8377082 | 0.749504 | 0.8347768 | 0.7928226 |
| sensitive nucleoside transporter | | | | | | | |
| Epidermal growth factor | 0.9965828 | 1.2178551 | 1.5044067 | 1.3998023 | 1.4302865 | 1.3989742 | 1.22446 |
| c-myc | 0.9444976 | 1.0541184 | 0.9413008 | 1.2570727 | 1.2432101 | 1.2089269 | 1.1163868 |
| Carbonic anhydrase III | 0.6231273 | 0.8345837 | 1.1372169 | 0.4795358 | 0.2686169 | 0.1849496 | 0.2028372 |
| Beta-actin | 1.4041195 | 1.2431613 | 1.1064727 | 1.1757787 | 0.9432045 | 1.2399414 | 0.9669534 |
| Bax (alpha) | 1.0653334 | 1.1471728 | 0.9755305 | 1.1394238 | 0.4988447 | 1.0986015 | 1.0887909 |
| Alpha 1 - inhibitor III | 0.5850105 | 1.1186627 | 1.1048896 | 0.7858807 | 0.552784 | 0.578092 | 0.5363697 |
| 60S ribosomal protein L6 | 1.1633608 | 1.3303285 | 1.1172074 | 1.246229 | 1.0751581 | 1.4194587 | 1.1461912 |
| Phase-1 RCT-117 | 0.8962536 | 0.9127982 | 1.5104566 | 1.3473152 | 1.296784 | 1.3626329 | 1.187847 |
| Phase-1 RCT-102 | 0.4843836 | 0.7290464 | 0.9393332 | 0.5187361 | 0.6335657 | 0.3928867 | 0.3343534 |
| 3-hydroxyisobutyrate dehydrogenase | 0.9041752 | 1.2934505 | 1.0470141 | 0.9515516 | 0.779919 | 0.927655 | 1.0004078 |
| ATPase inhibitor (rat mitochondrial IF1 | 0.91498 | 1.3097916 | 1.1264551 | 0.9819565 | 0.6893018 | 0.8239262 | 0.8331651 |
| protein) | | | | | | | |
| Alpha-2-macroglobulin | 1.1931322 | 1.398006 | 1.34228 | 0.9760035 | 1.5143198 | 3.7688572 | 3.641365 |
| Phase-1 RCT-137 | 0.9417717 | 1.1353768 | 1.0359992 | 0.9350271 | 0.7090305 | 0.8122262 | 0.9399968 |
| Phase-1 RCT 252 | 1.1561236 | 1.0807412 | 0.9486151 | 0.909766 | 0.7947239 | 0.8039961 | 0.9158813 |
| Phase-1 RCT-65 | 1.0949265 | 0.7253302 | 0.8041676 | 0.9139243 | 1.0784854 | 1.0185388 | 0.964945 |
| Liver fatty acid binding protein | 0.7032923 | 1.2566397 | 1.3157 | 0.9534683 | 0.364544 | 0.6956245 | 0.7067674 |
| Carbamyl phosphate synthetase I | 1.1761247 | 1.0537703 | 0.898905 | 0.8567108 | 0.7459986 | 0.8528347 | 0.9445739 |
| c-jun | 0.8561152 | 1.0576034 | 0.9763591 | 1.0821173 | 1.1570456 | 1.019149 | 1.0372682 |
| Transthyretin | 0.6639538 | 0.8416573 | 1.0226023 | 0.6438274 | 0.3471657 | 0.4123118 | 0.5059201 |
| Aflatoxin B1 aldehyde reductase | 0.7559094 | 0.9160531 | 0.8663881 | 0.803571 | 0.7299712 | 0.6515291 | 0.7900069 |
| Phase-1 RCT-15 | 1.2613454 | 0.9491328 | 0.9274648 | 0.9567491 | 0.8306106 | 0.9375537 | 0.9305009 |
| Insulin-like growth factor I | 0.6625483 | 1.0422248 | 1.1184182 | 0.884918 | 0.5654103 | 0.759491 | 0.7177323 |
| Sodium/bile acid cotransporter | 0.8068859 | 1.1025871 | 1.1035293 | 0.8227795 | 0.4676899 | 0.6407399 | 0.8295302 |
| Organic cation transporter 3 | 1.1304556 | 1.0360454 | 1.1485786 | 1.2880225 | 1.1503327 | 1.4467344 | 1.196245 |
| Gadd153 | 1.0613161 | 0.9964648 | 1.013136 | 1.1860693 | 1.1560538 | 1.1675631 | 1.0255171 |
| Phase-1 RCT-109 | 0.9617487 | 1.4736047 | 1.3388493 | 1.6066182 | 1.1075263 | 1.5332296 | 1.357719 |
| Phase-1 RCT-88 | 0.9411321 | 1.0049641 | 1.0259602 | 1 | 0.9749526 | 1.0264654 | 0.9517795 |
| Ubiquitin conjugating enzyme (RAD 6 | 1.1006974 | 1.4837803 | 1.2331097 | 1.4842604 | 1.1282028 | 1.2934452 | 1.485269 |
| homologue) | | | | | | | |
| Glucose-regulated protein 78 | 1.2428181 | 1.4143939 | 1.4555316 | 1.7375795 | 1.5070225 | 2.3957715 | 1.520934 |
| Ribosomal protein L13A | 0.9037868 | 2.1051333 | 1.2514815 | 1.6737267 | 1.3718643 | 1.7124127 | 1.5968841 |
| Monocyte chemotactic protein receptor (CCR2) | 1.0114678 | 0.8305488 | 1.102919 | 1.2375101 | 1.1789569 | 1.3779206 | 1.2171417 |
| Fas antigen | 1.1171663 | 1.1554203 | 1.3964238 | 1.6514571 | 1.0852808 | 1.5930628 | 1.3967149 |
| Calpactin I heavy chain | 1.0571114 | 0.7388207 | 0.8074206 | 0.953503 | 1.05044 | 1.1588517 | 0.9942155 |
| Uncoupling protein 2 | 0.857944 | 1.0927417 | 0.9452921 | 1.0785184 | 1.1166226 | 1.0573944 | 1.0679501 |
| Phase-1 RCT-168 | 1.0486772 | 1.0541917 | 1.2293272 | 1.2926707 | 1.0122651 | 1.2914813 | 1.0922012 |
| Phase-1 RCT-154 | 1.0315523 | 0.8123731 | 0.9002547 | 0.9411916 | 0.923289 | 0.9931874 | 1.0141993 |
| Superoxide dismutase Mn | 1.0026956 | 1.7931311 | 1.3136995 | 1.4579132 | 1.4575801 | 2.0912316 | 1.9115899 |
| Phase-1 RCT-214 | 0.9882354 | 1.1746662 | 1.2062724 | 1.1203108 | 0.9383328 | 1.0171969 | 1.2223859 |
| Phase-1 RCT-225 | 0.6388158 | 0.8663846 | 1.095809 | 0.9776422 | 1.0474476 | 1.0535673 | 1.0779146 |
| Phase-1 RCT-181 | 0.9887068 | 0.9058579 | 1.171667 | 1.1700705 | 1.0928146 | 1.0154092 | 1.0935737 |
| Nucleoside diphosphate kinase beta | 1.2793146 | 1.6095233 | 1.142993 | 1.4085383 | 1.1669791 | 1.5273639 | 1.5307045 |
| isoform | | | | | | | |
| Extracellular-signal-regulated kinase 1 | 0.7508411 | 0.8775373 | 1.0804611 | 0.6085772 | 0.4997078 | 0.327315 | 0.3236324 |
| {Ribosomal protein L6} | 1.1437004 | 1.2947508 | 1.0898371 | 1.21699 | 0.973102 | 1.3840315 | 1.1102005 |
| Aldehyde dehydrogenase, microsomal | 0.9128318 | 1.1903493 | 1.2996749 | 1.5529606 | 1.0317655 | 1.1258808 | 1.2268202 |
| p53 | 0.9518125 | 1.0585759 | 1.1299703 | 1.131571 | 1.154535 | 1.1306938 | 1.0153068 |
| Phase-1 RCT-239 | 0.9240445 | 0.7193955 | 0.7342554 | 0.8391464 | 0.9096711 | 0.8116496 | 0.7964684 |
| Macrophage inflammatory protein-2 alpha | 0.9907531 | 1.0770301 | 0.8797814 | 0.9835936 | 1.2305254 | 1.1006483 | 1.3060907 |
| IgE binding protein | 0.9285562 | 0.7619336 | 0.774358 | 0.8275303 | 0.9297729 | 1.0019716 | 1.0302432 |
| Phase-1 RCT-205 | 0.9971099 | 0.8310527 | 0.9160821 | 0.9445239 | 1.0272211 | 1.1299479 | 0.9905042 |
| Melanoma-associated antigen ME491 | 1.0413418 | 0.8933014 | 0.8894287 | 1.0837882 | 0.9710496 | 1.1542349 | 1.0019283 |
| Phase-1 RCT-242 | 0.9162872 | 0.8220349 | 0.8142368 | 0.8067749 | 0.8829714 | 0.8545437 | 0.8469437 |
| Enolase alpha | 1.9798956 | 1.5763352 | 1.3974018 | 1.5624995 | 2.196609 | 1.3272035 | 1.3155141 |
| Thymosin beta-10 | 0.9514072 | 1.4453231 | 1.1108308 | 1.275565 | 1.2182832 | 1.1645329 | 1.2355655 |
| Cofilin | 1.1772711 | 1.0556089 | 0.9019081 | 0.895893 | 0.662621 | 0.8204432 | 0.8248963 |
| Carbonyl reductase | 1.002473 | 0.9056512 | 0.9571164 | 0.9255852 | 0.9808534 | 0.881362 | 0.9532288 |
| MAP kinase kinase | 0.8919063 | 1.1302441 | 1.0801458 | 1.0635779 | 1.0474159 | 1.2201722 | 1.1015418 |
| Phase-1 RCT-162 | 1.0602555 | 0.9512007 | 1.0276769 | 1.0103471 | 0.8351822 | 1.2007734 | 1.1087223 |
| Phase-1 RCT-72 | 1.0338881 | 0.6708951 | 0.7182395 | 0.7508048 | 1.0462437 | 0.9083128 | 0.880863 |
| Ribosomal protein S8 | 1.1086005 | 1.3408228 | 1.108977 | 1.1859726 | 0.9787134 | 1.3853642 | 1.2583168 |
| Proliferating cell nuclear antigen gene | 0.8968966 | 0.9019503 | 0.8161073 | 0.8896762 | 1.0400723 | 0.9159782 | 0.9144177 |
| Phase-1 RCT-192 | 1.0831431 | 0.9545358 | 1.1163645 | 1.1802254 | 1.0971167 | 1.1932416 | 1.3586617 |
| IkB-a | 1.246955 | 1.3135934 | 1.0406927 | 1.1221793 | 0.7900329 | 1.0581249 | 1.0602478 |
| Endogenous retroviral sequence, 5′ and | 0.7676363 | 0.9419165 | 1.3551219 | 1.1728796 | 1.219649 | 1.439765 | 1.1936044 |
| 3′ LTR | | | | | | | |
| Phase-1 RCT-37 | 1.0946265 | 0.8393847 | 0.9969303 | 1.073692 | 0.9927762 | 1.241572 | 1.0928205 |
| Matrix metalloproteinase-1 | 1.0797313 | 1.3669475 | 1.0922022 | 1.0937433 | 0.9765365 | 1.2051444 | 1.1695774 |
| Cyclin G | 1.1426753 | 0.927368 | 0.9439414 | 1.0660563 | 1.2003452 | 1.1318227 | 1.0697398 |
| DNA polymerase beta | 1.0695652 | 1.1777807 | 0.9750892 | 1.0071118 | 0.8116791 | 0.916705 | 1.0304117 |
| Phase-1 RCT-127 | 0.9958279 | 0.7922245 | 1.0210559 | 0.9173634 | 1.1876384 | 1.1010038 | 0.9915028 |
| Multidrug resistant protein-2 | 0.880839 | 1.3267599 | 0.9625292 | 0.9865633 | 1.2161293 | 1.2592363 | 1.0002177 |
| Ribosomal protein S9 | 1.1062683 | 1.6887826 | 1.2279115 | 1.4277323 | 1.1858401 | 1.3671156 | 1.7134383 |
| Phase-1 RCT-12 | 1.0891831 | 0.872658 | 0.9102572 | 1.0254031 | 0.8052335 | 0.8811795 | 0.8661383 |
| Beta-tubulin, class I | 1.1518395 | 1.095425 | 0.89262 | 0.9807819 | 0.5908803 | 0.7195684 | 0.7875911 |
| Argininosuccinate lyase | 1.3661621 | 1.2406964 | 1.1377484 | 1.1941922 | 0.6948887 | 1.0419337 | 0.9429498 |
| Elongation factor-1 alpha | 1.0907369 | 1.4308841 | 0.9690195 | 1.0944589 | 0.8962008 | 1.093791 | 0.9550287 |
| Phase-1 RCT-55 | 1.0722129 | 0.6376712 | 0.7104994 | 0.8489142 | 1.0362318 | 1.0267856 | 0.8832764 |
| Macrophage inflammatory protein-1 alpha | 1.0156627 | 0.975791 | 0.8239509 | 0.7969764 | 1.0936152 | 1.0133909 | 1.0093943 |
| c-H-ras | 1.0889424 | 1.2574104 | 1.2048957 | 1.4656397 | 1.3118485 | 1.379995 | 1.4539462 |
| Multidrug resistant protein-1 | 0.9689362 | 1.2337555 | 1.0017997 | 1.1590967 | 1.0471948 | 1.2279593 | 1.0204519 |
| T-cell cyclophilin | 1.0741376 | 1.2705863 | 1.0031952 | 1.0170288 | 0.8173524 | 0.8995277 | 0.9672994 |
| Phase-1 RCT-185 | 0.7901897 | 1.147602 | 0.9235125 | 0.9084899 | 0.7178328 | 0.7532542 | 0.7056111 |
| 25-DX | 0.8720123 | 1.4570947 | 1.2995418 | 1.2592057 | 1.145478 | 1.0147473 | 1.1503934 |
| Ref-1 | 1.0787234 | 0.9177754 | 0.9881394 | 0.890492 | 0.8602657 | 1.1609738 | 1.0823809 |
| Apolipoprotein CIII | 0.8393125 | 0.9760724 | 1.1009572 | 1.054559 | 1.0141109 | 0.8587704 | 1.0457902 |
| NADPH quinone oxidoreductase-1 (DT- | 1.0603205 | 1.120312 | 0.9754635 | 0.8840929 | 0.9178307 | 1.2760704 | 1.4034709 |
| diaphorase) | | | | | | | |
| Pyruvate kinase, muscle | 0.9320664 | 0.7197864 | 0.7680998 | 0.902185 | 1.0471885 | 1.2491783 | 0.9838086 |
| | DEX | DEX | DEX | DEX | DEX | DEX | DIF |
| Compound-Dose (2) | 8 | 8 | 8 | 30 | 30 | 30 | 25 |
| Animal Number (3) | 1344 | 1345 | 1346 | 1354 | 1355 | 1356 | 244 |
| Liver Toxicity Necrosis Classification (4) | no | no | no | no | no | no | no |
| Gene Name (5) | | | | | | | |
| Phase-1 RCT-78 | 0.9016074 | 1.047702 | 0.9524189 | 0.8143447 | 0.8395853 | 0.9415394 | 1.2681366 |
| Gamma-actin, cytoplasmic | 1.0504767 | 0.9983521 | 1.3778216 | 1.2107049 | 1.0015125 | 1.1647716 | 0.9755414 |
| Matrin F/G | 1.1675302 | 1.2290709 | 1.233895 | 1.1326349 | 1.2895017 | 1.2039557 | 0.7044983 |
| Zinc finger protein | 0.9335774 | 0.9732305 | 0.944238 | 0.8712403 | 0.9913458 | 0.9490584 | 0.9500951 |
| Cathepsin L, sequence 2 | 1.1278893 | 0.7797205 | 1.0938381 | 1.1526314 | 1.1994022 | 0.9928828 | 0.8296714 |
| Gadd45 | 0.8988565 | 1.1327127 | 0.61942 | 0.9538803 | 0.751423 | 0.7880998 | 0.8870292 |
| Phase-1 RCT-144 | 1 | 1.0228603 | 1.0279365 | 1.0068527 | 0.9240651 | 0.997117 | 1.0155392 |
| Phase-1 RCT-145 | 1.0950401 | 1.1128432 | 1.0872256 | 1.0149844 | 1.0494667 | 1.035229 | 1.023923 |
| Phase-1 RCT-50 | 1.1780765 | 1.0487024 | 1.0769858 | 1.1814307 | 1.1186703 | 1.0143969 | 1.1409343 |
| Phase-1 RCT-92 | 1.4860952 | 0.9172064 | 0.9976957 | 1.0855561 | 1.1530167 | 1.2251908 | 1.2324997 |
| Phase-1 RCT-89 | 0.8632377 | 0.8299645 | 0.8372636 | 0.8369598 | 0.8691601 | 0.823132 | 0.8204704 |
| 14-3-3 zeta | 1.1247512 | 1.1677564 | 1.2318062 | 1.2250646 | 1.099514 | 1.220174 | 1.0377959 |
| Dynamin-1 (D100) | 1.0419639 | 0.8254351 | 0.9604431 | 0.9828914 | 0.9810203 | 1.0125628 | 0.8897803 |
| Insulin-like growth factor binding protein 1 | 0.9668605 | 0.9254602 | 1.0641761 | 1.0126386 | 0.9537184 | 0.973971 | 0.5834418 |
| L-gulono-gamma-lactone oxidase | 0.9605832 | 0.7559683 | 1.0206794 | 0.9258762 | 1.0382137 | 0.9731256 | 1.0264962 |
| Ornithine decarboxylase | 1.313035 | 1.2811236 | 1.3389722 | 2.094062 | 1.029919 | 1.1037291 | 1.9170657 |
| PAR interacting protein | 0.9578987 | 1.0499667 | 1.0014151 | 0.9535548 | 1.0086327 | 1.0243137 | 0.9978563 |
| Phase-1 RCT-128 | 1.0203365 | 0.8346393 | 0.9647681 | 0.9719711 | 1.0037056 | 1.0275246 | 0.8416228 |
| Phase-1 RCT-180 | 1.0317767 | 1.034779 | 1.1908523 | 1.1043671 | 1.0657126 | 1.0740765 | 0.9564257 |
| Phase-1 RCT-182 | 1.0905079 | 0.9612501 | 0.935752 | 0.8883732 | 0.8958973 | 0.9529584 | 1.0180123 |
| Phase-1 RCT-207 | 1.4934056 | 1.1954647 | 1.4177564 | 1.9695834 | 1.4322838 | 1.6315132 | 0.8998502 |
| Phase-1 RCT-213 | 1.4176707 | 1.1132253 | 1.3200431 | 1.9172125 | 1.4247601 | 1.5040355 | 0.8700756 |
| Phase-1 RCT-256 | 0.9612078 | 0.9343951 | 0.9522353 | 0.9981769 | 1.1777793 | 1.0157566 | 0.9593858 |
| Phase-1 RCT-258 | 0.9640819 | 1.0029348 | 1.0519202 | 1.0480452 | 1.0738331 | 1.0845735 | 0.8694243 |
| Phase-1 RCT-264 | 1.3019388 | 1.2222999 | 0.9791482 | 1.0882916 | 0.9927167 | 0.8856316 | 1.4883032 |
| Phase-1 RCT-271 | 0.952562 | 0.8414075 | 0.9799365 | 1.1851102 | 1.055208 | 0.9271801 | 1.2774917 |
| Phase-1 RCT-288 | 0.9436787 | 0.8805234 | 0.7579634 | 0.8613958 | 1.0593352 | 0.9401083 | 0.7449552 |
| Phase-1 RCT-38 | 1.079758 | 0.8578972 | 0.9523553 | 1.0642976 | 1.2117375 | 1.044451 | 0.8952225 |
| Phase-1 RCT-39 | 1.0508671 | 0.984838 | 1.0810317 | 1.2475129 | 1.1666921 | 1.104852 | 0.9003261 |
| Phase-1 RCT-68 | 1.2886081 | 1.1694928 | 1.2695768 | 1.4197143 | 1.161459 | 1.1425786 | 1.0827061 |
| Phase-1 RCT-33 | 0.8485021 | 0.837167 | 0.8621183 | 0.7999304 | 0.8486944 | 0.9205629 | 0.6922109 |
| Phase-1 RCT-36 | 0.7668174 | 0.9716932 | 0.9411978 | 0.7624822 | 0.8541378 | 0.9035047 | 0.8985426 |
| 17-beta hydroxysteroid dehydrogenase, | 0.2631113 | 1.5418183 | 0.2737214 | 0.3835117 | 0.3544665 | 0.3098543 | 0.3467 |
| type 2 | | | | | | | |
| Senescence marker protein-30 | 0.1751853 | 0.5998646 | 0.2439391 | 0.0909845 | 0.1246195 | 0.1577867 | 1.0460982 |
| Ribosomal protein S17 | 1.0611134 | 0.8206289 | 0.9380397 | 1.0008417 | 0.8710777 | 0.8536615 | 0.9029493 |
| Phase-1 RCT-83 | 0.590431 | 0.7868895 | 0.525788 | 0.3819335 | 0.5376735 | 0.5923828 | 1.0104488 |
| Phase-1 RCT-49 | 0.8587477 | 1.0142572 | 0.9212242 | 0.8948476 | 0.8914739 | 0.945809 | 0.9605337 |
| Phase-1 RCT-48 | 0.8457468 | 0.8018658 | 1.1125082 | 1.0186676 | 0.9386842 | 1.0271327 | 1.382412 |
| Phase-1 RCT-40 | 0.9237121 | 0.9971122 | 0.9367893 | 1.0670631 | 1.0553341 | 0.9755881 | 0.9225808 |
| Phase-1 RCT-296 | 0.5101435 | 0.7218407 | 0.3445743 | 0.5133583 | 0.4550724 | 0.5362017 | 0.8579184 |
| Phase-1 RCT-295 | 1.1768588 | 1.0431111 | 1.1205034 | 1.2532243 | 1.1271898 | 1.0429481 | 0.966849 |
| Phase-1 RCT-291 | 1.1561921 | 0.9001495 | 1.1296004 | 1.1174682 | 1.157917 | 1.076194 | 1.0858755 |
| Phase-1 RCT-270 | 0.8232746 | 1.0886072 | 0.8980894 | 0.9614574 | 0.8721803 | 0.8733677 | 0.9461792 |
| Phase-1 RCT-241 | 1.0306824 | 0.9649197 | 1.0331721 | 1.0325253 | 1.0312617 | 1.0729878 | 0.8739892 |
| Phase-1 RCT-191 | 0.8987376 | 1.1407268 | 1.1853619 | 1.1184291 | 0.9850183 | 0.8941214 | 1.2241559 |
| Phase-1 RCT-189 | 0.9410226 | 1.3702569 | 1.2251792 | 0.9656558 | 1.2688469 | 0.9710921 | 1.0621101 |
| Phase-1 RCT-179 | 0.9436284 | 0.8885917 | 0.7789137 | 0.8280183 | 0.9782131 | 0.8568198 | 0.7720468 |
| Phase-1 RCT-152 | 1.0188305 | 0.8409632 | 0.8516023 | 0.9838529 | 0.9107038 | 0.9069822 | 0.7159046 |
| Phase-1 RCT-123 | 1.0932989 | 1.0736104 | 0.9703695 | 1.1874835 | 1.068932 | 1.0515445 | 1.1134479 |
| Paraoxonase 1 | 0.5693803 | 0.7328787 | 0.5723285 | 0.4837934 | 0.6075611 | 0.5960118 | 0.9797956 |
| Organic anion transporter 3 | 1.2515669 | 0.8646781 | 0.7035663 | 1.2976725 | 1.0973067 | 1.0953355 | 1.2188452 |
| N-hydroxy-2-acetylaminofluorene | 0.7097396 | 0.7082622 | 0.4748208 | 0.4180065 | 0.5772609 | 0.6181135 | 0.9590185 |
| sulfotransferase (ST1C1) | | | | | | | |
| Integrin beta1 | 1.3321288 | 1.1186386 | 1.1327616 | 1.4544283 | 1.1433564 | 1.1473787 | 0.9149853 |
| Insulin-like growth factor binding protein 3 | 0.8270477 | 1.0142798 | 0.7618303 | 0.8708798 | 0.8253288 | 0.9937779 | 0.8737868 |
| ID-1 | 1.172157 | 1.1538845 | 1.160872 | 1.1738592 | 1.1933732 | 1.1359069 | 1.1631093 |
| Hepatic lipase | 1.0539937 | 0.662697 | 1.124715 | 0.8159776 | 1.2627561 | 1.3783836 | 0.6951066 |
| Heme oxygenase | 0.5271825 | 1.1013604 | 0.7973837 | 0.6117483 | 0.6303521 | 0.5957731 | 0.9149236 |
| Equilbrative nitrobenzylthioinosine- | 0.6147467 | 0.7964698 | 0.5684933 | 0.3963633 | 0.5255498 | 0.6012438 | 0.990347 |
| sensitive nucleoside transporter | | | | | | | |
| Epidermal growth factor | 0.8202153 | 0.96982 | 0.7690861 | 0.9336134 | 0.7689949 | 0.7333729 | 0.9944014 |
| c-myc | 1.2691678 | 1.3219734 | 1.4357251 | 1.4392366 | 1.5724199 | 1.5278071 | 1.1741662 |
| Carbonic anhydrase III | 0.2070855 | 0.6654532 | 0.1586079 | 0.0860625 | 0.1409493 | 0.1500412 | 1.2480069 |
| Beta-actin | 1.0199512 | 1.0837424 | 1.2858558 | 1.9535304 | 1.0538042 | 1.4361104 | 0.9605029 |
| Bax (alpha) | 1.3836203 | 1.5180525 | 1.557838 | 1.5934169 | 1.464582 | 1.5635858 | 1.2806216 |
| Alpha 1 - inhibitor III | 0.5170906 | 1.1284292 | 0.6118412 | 0.4955207 | 0.643531 | 0.5539833 | 1.4102213 |
| 60S ribosomal protein L6 | 1.1265074 | 0.8783677 | 0.9883308 | 1.2490304 | 1.1080885 | 0.982097 | 0.7789582 |
| Phase-1 RCT-117 | 0.8835576 | 0.7045741 | 0.8635302 | 0.6457098 | 0.8530837 | 0.6992543 | 0.9614768 |
| Phase-1 RCT-102 | 0.6534954 | 0.9108908 | 0.6782553 | 0.5160103 | 0.6908931 | 0.7054934 | 0.927115 |
| 3-hydroxyisobutyrate dehydrogenase | 0.7364823 | 0.8431144 | 0.8473383 | 0.7373608 | 0.9651749 | 0.6221082 | 1.0493352 |
| ATPase inhibitor (rat mitochondrial IF1 | 0.5599418 | 0.7400284 | 0.5244647 | 0.3873609 | 0.4856478 | 0.5069025 | 0.7114059 |
| protein) | | | | | | | |
| Alpha-2-macroglobulin | 0.4673016 | 1.0138469 | 0.6150506 | 0.5416076 | 0.5276723 | 0.5899175 | 1.1513122 |
| Phase-1 RCT-137 | 0.6255344 | 0.6574938 | 0.6263865 | 0.5995761 | 0.6085627 | 0.5601373 | 0.8410449 |
| Phase-1 RCT 252 | 1.6648535 | 0.9326198 | 1.4003679 | 1.9796063 | 1.9868429 | 1.7242062 | 0.7312009 |
| Phase-1 RCT-65 | 1.1604254 | 1.2968013 | 1.362569 | 1.3287864 | 1.3063271 | 1.3071054 | 1.1113621 |
| Liver fatty acid binding protein | 0.5984197 | 0.8346289 | 0.509371 | 0.3897052 | 0.4439674 | 0.4668869 | 0.7139377 |
| Carbamyl phosphate synthetase I | 1.816814 | 0.9287933 | 1.7339363 | 2.3461616 | 2.593192 | 2.0618658 | 0.7920148 |
| c-jun | 1.4035677 | 1.3333827 | 1.3333013 | 1.7341735 | 1.3622386 | 1.3648615 | 1.1261715 |
| Transthyretin | 0.5974794 | 1.007511 | 0.6963937 | 0.6509537 | 0.5647217 | 0.6017239 | 1.2602017 |
| Aflatoxin B1 aldehyde reductase | 0.9248796 | 0.9363239 | 0.9716532 | 0.9632127 | 0.9718555 | 0.9466749 | 0.72762 |
| Phase-1 RCT-15 | 1.3656176 | 1.1912901 | 1.4050114 | 1.2931926 | 1.4245993 | 1.1770061 | 1.4862294 |
| Insulin-like growth factor I | 0.8007879 | 0.9478875 | 0.6959159 | 0.8381752 | 1 | 1.0281444 | 0.740289 |
| Sodium/bile acid cotransporter | 0.4314776 | 0.6649001 | 0.5301635 | 0.5905081 | 0.3612221 | 0.4591997 | 0.6875928 |
| Organic cation transporter 3 | 1.1565741 | 0.8640005 | 1.001734 | 1.2119286 | 1.1061155 | 0.9716686 | 0.7685725 |
| Gadd153 | 1.2919445 | 1.1944822 | 1.1410551 | 1.4994215 | 1.1878908 | 1.3056116 | 1.2064006 |
| Phase-1 RCT-109 | 1.1631279 | 0.9125801 | 1.0456504 | 1.184129 | 1.0023848 | 1.0825773 | 0.6204044 |
| Phase-1 RCT-88 | 1.5889429 | 0.9663833 | 0.8911583 | 0.9731609 | 1.1609735 | 1.240619 | 1.1831139 |
| Ubiquitin conjugating enzyme (RAD 6 | 1.1554683 | 0.9008179 | 0.978698 | 1.2124301 | 1.0773935 | 1.0196687 | 0.9668307 |
| homologue) | | | | | | | |
| Glucose-regulated protein 78 | 1.4283929 | 0.7986428 | 1.1380045 | 1.2771454 | 1.0092942 | 1.2631692 | 0.6820516 |
| Ribosomal protein L13A | 1.2663463 | 1.1912576 | 1.2523277 | 1.1856581 | 1.3362538 | 1.2243726 | 0.772004 |
| Monocyte chemotactic protein receptor (CCR2) | 1.1622503 | 0.9122417 | 1.0536101 | 1.2337668 | 1.118325 | 1.0058485 | 0.9711044 |
| Fas antigen | 1.1093762 | 1.0405878 | 1.209605 | 1.3190597 | 1.0495738 | 1.2463725 | 0.8307147 |
| Calpactin I heavy chain | 1.1537789 | 1.1704319 | 1.3028935 | 1.5996423 | 1.1911826 | 1.1222261 | 1.208356 |
| Uncoupling protein 2 | 1.3977586 | 0.9337968 | 1.1758285 | 1.2180458 | 1.4078048 | 1.372554 | 0.6901072 |
| Phase-1 RCT-168 | 0.8313901 | 1.0215636 | 0.9069839 | 0.7707748 | 0.9123206 | 0.8938018 | 0.8798043 |
| Phase-1 RCT-154 | 1.1175152 | 1.089165 | 1.0468183 | 1.0385841 | 1.0408214 | 1.0267228 | 0.9850259 |
| Superoxide dismutase Mn | 1.3760265 | 1.1935889 | 1.2892783 | 1.5199355 | 1.2704139 | 1.2275697 | 1.1046427 |
| Phase-1 RCT-214 | 1.0542988 | 0.9638431 | 1.0236752 | 0.91697 | 1.1251256 | 0.9553995 | 1.0360509 |
| Phase-1 RCT-225 | 0.7049421 | 1.0897186 | 0.8227121 | 0.6441245 | 0.6502419 | 0.5730928 | 1.076926 |
| Phase-1 RCT-181 | 1.1147672 | 0.9761971 | 0.9691011 | 0.9829947 | 1.0906028 | 1.1257753 | 1.0112641 |
| Nucleoside diphosphate kinase beta | 1.0921172 | 0.882027 | 1.1286196 | 1.3731439 | 1.1275012 | 1.0549165 | 1.0344825 |
| isoform | | | | | | | |
| Extracellular-signal-regulated kinase 1 | 0.173137 | 0.6937633 | 0.199679 | 0.1047352 | 0.150082 | 0.2109335 | 1.3114012 |
| {Ribosomal protein L6} | 1.864099 | 0.9601462 | 1.3303138 | 1.6762054 | 1.7611854 | 1.5706056 | 0.8197972 |
| Aldehyde dehydrogenase, microsomal | 0.9280436 | 1.0977849 | 1.0591354 | 0.8925377 | 1.0320721 | 1.001144 | 0.7448703 |
| p53 | 1.0591621 | 0.9082669 | 0.996808 | 0.8635985 | 0.9672056 | 1.0110146 | 0.9712527 |
| Phase-1 RCT-239 | 1.2491986 | 1.3014317 | 1.5640558 | 1.8845156 | 1.7338325 | 1.5355486 | 1.2516769 |
| Macrophage inflammatory protein-2 alpha | 0.8939349 | 0.9056923 | 0.8391192 | 0.9527214 | 0.9643906 | 0.7477584 | 0.9088844 |
| IgE binding protein | 0.9625534 | 1.1119753 | 0.9968702 | 1.06081 | 1.141606 | 0.9625751 | 1.0188591 |
| Phase-1 RCT-205 | 1.0267532 | 1.0100111 | 1.0203888 | 1.0370837 | 0.9499991 | 1.0034962 | 1.0777103 |
| Melanoma-associated antigen ME491 | 0.867502 | 1.1649553 | 0.9642277 | 0.9439852 | 0.8628026 | 0.8657712 | 1.2606784 |
| Phase-1 RCT-242 | 1.1116986 | 1.1538302 | 1.100549 | 1.3228401 | 1.3140354 | 1.2169123 | 1.1442951 |
| Enolase alpha | 1.3559322 | 0.7556119 | 1.113777 | 1.0604686 | 0.9498641 | 0.89166 | 0.6110686 |
| Thymosin beta-10 | 1.5120194 | 0.8367428 | 1.6898222 | 1.7339818 | 2.097716 | 1.8710811 | 0.7017548 |
| Cofilin | 0.926928 | 1.0218201 | 0.9282548 | 0.9462159 | 1.0848842 | 0.9711912 | 1.4546252 |
| Carbonyl reductase | 1.1022089 | 1.0038049 | 1.0690098 | 1.2480377 | 1.0275369 | 1.1888652 | 1.0489371 |
| MAP kinase kinase | 0.8850357 | 1.043164 | 1.0269703 | 1.1179563 | 0.8707532 | 0.8949223 | 0.9054016 |
| Phase-1 RCT-162 | 1.010651 | 0.8486154 | 0.8767442 | 1.0985435 | 1.0168097 | 0.8220152 | 0.9135545 |
| Phase-1 RCT-72 | 1.2194732 | 1.0864666 | 1.0833468 | 1.3614608 | 1.1461796 | 1.0630473 | 0.9275221 |
| Ribosomal protein S8 | 0.9544565 | 0.7927226 | 0.9010562 | 1.0480707 | 1.0173916 | 0.8002484 | 0.7593466 |
| Proliferating cell nuclear antigen gene | 1.0936941 | 1.1237994 | 1.0278995 | 1.0928977 | 0.9930566 | 1.0600398 | 1.059154 |
| Phase-1 RCT-192 | 0.9336989 | 0.8207323 | 0.8912228 | 0.9132519 | 0.911441 | 0.8665707 | 0.8455856 |
| IkB-a | 1.2772547 | 0.8444852 | 1.2714325 | 1.331598 | 1.2245946 | 1.1809969 | 0.9714178 |
| Endogenous retroviral sequence, 5′ and | 1.485529 | 0.895126 | 0.9100264 | 0.9278592 | 1.1088786 | 0.9358371 | 1.0346006 |
| 3′ LTR | | | | | | | |
| Phase-1 RCT-37 | 1.4140047 | 0.9580507 | 1.3021775 | 1.6177237 | 1.5932928 | 1.3942528 | 0.9242801 |
| Matrix metalloproteinase-1 | 1.5199137 | 1.1679509 | 1.2021229 | 1.5901262 | 1.4906188 | 1.2735887 | 0.819165 |
| Cyclin G | 1.348115 | 1.2312993 | 1.1527976 | 1.892217 | 1.0820698 | 1.1879805 | 1.2959547 |
| DNA polymerase beta | 1.0092962 | 0.8329693 | 0.94353 | 0.9656296 | 0.9207724 | 0.9001613 | 1.1393579 |
| Phase-1 RCT-127 | 0.9043407 | 0.8059779 | 0.8695816 | 0.6592069 | 0.8559969 | 0.6951018 | 0.9762267 |
| Multidrug resistant protein-2 | 1.390414 | 1.361758 | 1.3981218 | 1.5249064 | 1.3481936 | 1.3526287 | 1.02338 |
| Ribosomal protein S9 | 1.1485845 | 0.9454855 | 1.0321845 | 1.2294197 | 1.3326836 | 1.3074819 | 1.0141214 |
| Phase-1 RCT-12 | 1.1748898 | 0.8683463 | 1.2869905 | 1.5661703 | 1.227762 | 1.2866415 | 1.1713469 |
| Beta-tubulin, class I | 1.0709851 | 0.7036567 | 1.5008382 | 1.5112339 | 1.2313545 | 1.233854 | 1.2451168 |
| Argininosuccinate lyase | 1.0436952 | 1.2862256 | 1.1258215 | 1.0457597 | 0.9892073 | 0.8361496 | 1.073455 |
| Elongation factor-1 alpha | 1.1787244 | 1.0493758 | 1.0925428 | 1.0840623 | 1.2337308 | 1.0939821 | 1.0740578 |
| Phase-1 RCT-55 | 1.0665163 | 1.0354108 | 1.2874337 | 1.7527561 | 0.9855029 | 1.1599869 | 0.9992306 |
| Macrophage inflammatory protein-1 alpha | 1.1137325 | 1.1383218 | 1.1026648 | 1.2039791 | 1.1821076 | 1.1495212 | 0.9966862 |
| c-H-ras | 0.7347537 | 0.9424608 | 0.8674112 | 0.804673 | 0.7479881 | 0.7030091 | 0.9350114 |
| Multidrug resistant protein-1 | 1.7038599 | 1.5688658 | 1.5602114 | 2.012833 | 1.7821885 | 1.5754657 | 1.3874929 |
| T-cell cyclophilin | 1.0172763 | 0.7793455 | 0.9399415 | 0.959794 | 0.9604566 | 0.920778 | 1.0542505 |
| Phase-1 RCT-185 | 0.8959007 | 0.8912553 | 0.7220727 | 0.6482225 | 0.7576036 | 0.6823117 | 1.3714492 |
| 25-DX | 0.6744704 | 0.7847038 | 0.6292614 | 0.7262102 | 0.6577414 | 0.6937497 | 0.698531 |
| Ref-1 | 0.8625024 | 0.824925 | 0.7279568 | 0.9420822 | 0.6306458 | 0.7546803 | 0.7516345 |
| Apolipoprotein CIII | 1.0097816 | 1.0441012 | 0.9882657 | 0.8132122 | 0.9867947 | 0.9899838 | 1.2155291 |
| NADPH quinone oxidoreductase-1 (DT- | 0.799555 | 0.7174253 | 0.6483225 | 0.8673986 | 0.5332554 | 0.6387594 | 0.7009314 |
| diaphorase) | | | | | | | |
| Pyruvate kinase, muscle | 1.2887633 | 0.9414723 | 1.2636769 | 1.2105726 | 1.2433457 | 1.1593304 | 1.0172545 |
| | DIF | DIF | DIF | DIF | DIF | DOX | DOX |
| Compound-Dose (2) | 25 | 25 | 100 | 100 | 100 | 12 | 12 |
| Animal Number (3) | 245 | 246 | 254 | 255 | 256 | 1254 | 1255 |
| Liver Toxicity Necrosis Classification (4) | no | no | no | no | no | no | no |
| Gene Name (5) | | | | | | | |
| Phase-1 RCT-78 | 1.0054765 | 1.0562277 | 1.1915442 | 1.0790083 | 1.058604 | 0.9839231 | 1.0131279 |
| Gamma-actin, cytoplasmic | 1.1522331 | 0.9845947 | 0.9753384 | 0.9208074 | 0.9002559 | 0.9334328 | 0.7112135 |
| Matrin F/G | 0.7401431 | 0.8223853 | 0.9067551 | 0.9304595 | 0.9191722 | 0.7384959 | 0.9549521 |
| Zinc finger protein | 0.9181842 | 0.9336498 | 0.8931084 | 1.0458792 | 0.9855228 | 1.0369691 | 0.9658368 |
| Cathepsin L, sequence 2 | 0.812287 | 0.6718458 | 0.9307667 | 0.9211901 | 0.8307942 | 1.4108573 | 0.9150273 |
| Gadd45 | 1.0341849 | 1.0453503 | 0.7287909 | 0.9086292 | 0.8121401 | 1.2922177 | 1.4613928 |
| Phase-1 RCT-144 | 1.1018745 | 1.1295192 | 1.2953196 | 1.1583499 | 1.1585755 | 1.124259 | 1.0434958 |
| Phase-1 RCT-145 | 1.1503525 | 1.1301882 | 1.0904553 | 1.2827536 | 1.1539549 | 1.5768801 | 1.1469966 |
| Phase-1 RCT-50 | 1.0833458 | 1.3020625 | 1.0947018 | 1.1134381 | 1.1388752 | 0.8861762 | 0.947103 |
| Phase-1 RCT-92 | 1.1045401 | 0.9797983 | 0.9841723 | 0.7470164 | 0.9490318 | 0.7962785 | 0.7303543 |
| Phase-1 RCT-89 | 0.8428074 | 0.7928727 | 0.9959372 | 0.8756568 | 0.8625919 | 0.6073347 | 0.7344323 |
| 14-3-3 zeta | 0.9862049 | 1.0677679 | 1.1530576 | 1.2453339 | 1.0753685 | 1.2606515 | 1.101815 |
| Dynamin-1 (D100) | 0.8956808 | 0.7801103 | 0.9161274 | 0.7210956 | 0.8577027 | 0.7402272 | 0.8494076 |
| Insulin-like growth factor binding protein 1 | 0.6609483 | 0.6840662 | 0.6237623 | 0.7076913 | 0.655158 | 1.3940378 | 0.9862287 |
| L-gulono-gamma-lactone oxidase | 1.2316203 | 1.3397641 | 0.921728 | 1.2478249 | 1.0314366 | 0.6313162 | 0.5920211 |
| Ornithine decarboxylase | 1.9348685 | 2.5246766 | 1.7744201 | 2.3055675 | 2.5618808 | 0.7645056 | 0.9960042 |
| PAR interacting protein | 1.0712777 | 1.0350841 | 1.2932757 | 1.3033093 | 1.1574073 | 1.3560556 | 1.0963247 |
| Phase-1 RCT-128 | 0.9115381 | 0.6487989 | 0.9075577 | 0.7279741 | 0.8744426 | 0.6109814 | 0.8298952 |
| Phase-1 RCT-180 | 1.1608938 | 1.1655883 | 0.8552088 | 1.0576811 | 1.0319508 | 0.9423754 | 0.9808528 |
| Phase-1 RCT-182 | 1.0299796 | 0.8145503 | 1.1357232 | 0.96543 | 0.9188481 | 0.8581695 | 0.8095816 |
| Phase-1 RCT-207 | 0.931024 | 1.0766408 | 0.7467794 | 0.9488957 | 1.077221 | 3.9848044 | 1.1706065 |
| Phase-1 RCT-213 | 0.9745477 | 1.0657272 | 0.9338867 | 0.9499033 | 1.0747455 | 1.5059562 | 1.2296436 |
| Phase-1 RCT-256 | 0.9674627 | 0.9182549 | 0.9607895 | 1.0270376 | 0.9879929 | 0.6277827 | 0.7594094 |
| Phase-1 RCT-258 | 0.9436733 | 0.9757629 | 0.8888035 | 1.067213 | 1.0447125 | 1.0262396 | 1.0742414 |
| Phase-1 RCT-264 | 0.9904245 | 0.7112802 | 1.4137381 | 1.2208863 | 1.281418 | 0.9826869 | 0.7432373 |
| Phase-1 RCT-271 | 1.262436 | 1.3997965 | 0.9441074 | 1.1792747 | 1.0024462 | 0.7188248 | 0.6880351 |
| Phase-1 RCT-288 | 0.7463127 | 0.6018459 | 0.7746908 | 0.8344995 | 0.9327966 | 0.6963155 | 0.651479 |
| Phase-1 RCT-38 | 0.9016998 | 0.8141939 | 0.9931758 | 0.9538496 | 0.9665953 | 0.6593506 | 0.7590686 |
| Phase-1 RCT-39 | 0.9264361 | 0.9241397 | 0.8268709 | 1.0536855 | 1.0622908 | 1.2347424 | 0.9394559 |
| Phase-1 RCT-68 | 1.0981326 | 1.2007613 | 1.1402569 | 1.221857 | 1.1311085 | 0.9997644 | 1.2364086 |
| Phase-1 RCT-33 | 0.8122988 | 0.9065056 | 1.1020057 | 0.9430744 | 0.9053095 | 1.0724941 | 0.7772337 |
| Phase-1 RCT-36 | 0.9391925 | 1.0875279 | 1.0932105 | 0.9253519 | 0.9847777 | 0.9433672 | 0.7948577 |
| 17-beta hydroxysteroid dehydrogenase, | 0.460961 | 0.6105518 | 0.4312654 | 0.8859535 | 0.7838344 | 5.6818852 | 1.8134654 |
| type 2 | | | | | | | |
| Senescence marker protein-30 | 1.1221726 | 0.6569196 | 1.1026238 | 1.0750107 | 0.907983 | 0.6753206 | 1.0680922 |
| Ribosomal protein S17 | 0.9109137 | 0.7271886 | 1.2865689 | 1.2448672 | 1.1970538 | 1.2216486 | 1.2405282 |
| Phase-1 RCT-83 | 0.8350925 | 0.7869297 | 0.9791799 | 0.8645746 | 0.7540507 | 0.6750171 | 0.8116761 |
| Phase-1 RCT-49 | 0.9788159 | 1.0496899 | 1.1463145 | 1.0586997 | 1.0639055 | 1.0551763 | 0.9813357 |
| Phase-1 RCT-48 | 1.2894795 | 1.1010865 | 1.5748872 | 1.8059895 | 1.4936033 | 0.6825086 | 1.0942042 |
| Phase-1 RCT-40 | 1.0001905 | 0.7252309 | 1.1329285 | 0.8846656 | 1.0477209 | 0.7632098 | 0.8540155 |
| Phase-1 RCT-296 | 0.906794 | 0.6310231 | 1.0353827 | 1.0394692 | 1.0207493 | 0.9363716 | 0.3294354 |
| Phase-1 RCT-295 | 0.9548269 | 0.8963004 | 1.0214832 | 1.0470282 | 0.9984825 | 1.1534892 | 1.1554133 |
| Phase-1 RCT-291 | 1.0995327 | 1.0076678 | 1.1619645 | 1.1672877 | 1.0681014 | 0.6345833 | 0.8505756 |
| Phase-1 RCT-270 | 0.9806274 | 0.8760611 | 1.1227957 | 0.8906972 | 1.0871042 | 0.6331319 | 0.7512263 |
| Phase-1 RCT-241 | 0.9654019 | 1.0652418 | 0.802194 | 0.9622636 | 1.0498322 | 1.071882 | 1.3263177 |
| Phase-1 RCT-191 | 1.3596039 | 1.8359603 | 1.1009502 | 1.1807156 | 0.8712906 | 0.8123763 | 0.730506 |
| Phase-1 RCT-189 | 1.0649163 | 1.0044433 | 0.9457858 | 0.9228458 | 0.8679664 | 0.8114274 | 0.9105365 |
| Phase-1 RCT-179 | 0.8935874 | 0.7074423 | 0.8733484 | 0.8279275 | 0.8531704 | 1.3963594 | 1.0014902 |
| Phase-1 RCT-152 | 0.782513 | 0.6566529 | 0.8827272 | 0.7603868 | 0.7129391 | 1.4389966 | 1.3007702 |
| Phase-1 RCT-123 | 1.0472156 | 1.2295187 | 1.0866455 | 0.9923956 | 1.025285 | 0.9031745 | 0.9819815 |
| Paraoxonase 1 | 0.8334145 | 0.5565577 | 0.7970424 | 0.7382121 | 0.7014539 | 0.9174501 | 0.9899404 |
| Organic anion transporter 3 | 1.1004206 | 1.070036 | 1.0221021 | 1.0430906 | 1.0006036 | 1.033883 | 0.9407284 |
| N-hydroxy-2-acetylaminofluorene | 0.7522546 | 0.6366667 | 0.968888 | 0.8405594 | 0.7602168 | 0.7659894 | 0.9505844 |
| sulfotransferase (ST1C1) | | | | | | | |
| Integrin beta1 | 0.8543854 | 0.9904251 | 0.8444621 | 0.8628562 | 0.8413037 | 1.1281446 | 1.2119812 |
| Insulin-like growth factor binding protein 3 | 0.8159961 | 0.6226863 | 0.8316076 | 0.7562817 | 0.7814363 | 0.9809638 | 1.054671 |
| ID-1 | 1.1715382 | 1.250535 | 0.9216662 | 0.9525418 | 1.0086107 | 1.5117502 | 1.5514625 |
| Hepatic lipase | 1.0688205 | 0.8213968 | 0.8180063 | 0.7073626 | 0.7610552 | 0.638737 | 1.1117998 |
| Heme oxygenase | 1.2185714 | 0.9342359 | 0.8846934 | 1.0408093 | 0.8662071 | 1.064681 | 1.1228601 |
| Equilbrative nitrobenzylthioinosine- | 0.8051648 | 0.7997835 | 0.9942466 | 0.8984562 | 0.7952143 | 0.7094553 | 0.8872644 |
| sensitive nucleoside transporter | | | | | | | |
| Epidermal growth factor | 0.7609399 | 0.5475023 | 0.7998418 | 0.7768145 | 0.634099 | 1.035831 | 2.1082518 |
| c-myc | 1.174976 | 1.5797335 | 1.186149 | 1.4352084 | 1.3868939 | 0.9767461 | 1.0438954 |
| Carbonic anhydrase III | 1.3518391 | 0.8745595 | 2.609636 | 1.415605 | 1.0263969 | 0.6678135 | 0.3842935 |
| Beta-actin | 1.0174677 | 0.6884565 | 1.1876681 | 0.9515724 | 0.9313752 | 1.0399488 | 1.1563126 |
| Bax (alpha) | 1.2574457 | 1.7794076 | 1.3011365 | 1.5375423 | 1.6359781 | 1.2937338 | 1.4070051 |
| Alpha 1 - inhibitor III | 1.1390623 | 0.8318231 | 1.4007106 | 0.8227961 | 1.1251445 | 0.9734188 | 0.776804 |
| 60S ribosomal protein L6 | 0.8656142 | 0.7029315 | 0.8469934 | 0.9274637 | 0.9005132 | 1.4460454 | 1.5024986 |
| Phase-1 RCT-117 | 1.1962621 | 1.1519722 | 0.7961746 | 1.4468807 | 1.3580258 | 0.4775811 | 1.1952397 |
| Phase-1 RCT-102 | 0.976449 | 0.5812911 | 0.7926087 | 0.9841508 | 0.8825305 | 0.3675549 | 0.4223357 |
| 3-hydroxyisobutyrate dehydrogenase | 1.0291436 | 0.9002419 | 1.094336 | 1.0302578 | 0.8928766 | 0.8606284 | 0.8550839 |
| ATPase inhibitor (rat mitochondrial IF1 | 0.7100032 | 0.5270888 | 0.6933163 | 0.7452415 | 0.5762413 | 0.6924334 | 1.2469176 |
| protein) | | | | | | | |
| Alpha-2-macroglobulin | 1.0731332 | 0.8742707 | 1.2757528 | 0.8887965 | 0.8876357 | 1.2667946 | 1.0870404 |
| Phase-1 RCT-137 | 1.0189394 | 0.7794142 | 0.8725105 | 0.952718 | 0.8068268 | 0.8455533 | 1.1713247 |
| Phase-1 RCT 252 | 0.8650051 | 0.740093 | 0.7305729 | 0.7376211 | 0.6951842 | 0.676213 | 0.6447738 |
| Phase-1 RCT-65 | 1.0977235 | 1.5055437 | 1.0402446 | 1.179867 | 1.100142 | 0.8343763 | 0.8512572 |
| Liver fatty acid binding protein | 0.7581012 | 0.5145845 | 0.6751255 | 0.7179415 | 0.8231087 | 1.3176926 | 0.8149537 |
| Carbamyl phosphate synthetase I | 0.9317059 | 0.7818984 | 0.7565774 | 0.750462 | 0.6796513 | 0.6075342 | 0.5835581 |
| c-jun | 1.1144154 | 1.4459878 | 1.0828898 | 1.2425681 | 1.3008494 | 1.9220823 | 1.1018791 |
| Transthyretin | 0.8081868 | 0.5503009 | 0.7018675 | 0.5688645 | 0.6222885 | 0.8652858 | 0.6974813 |
| Aflatoxin B1 aldehyde reductase | 0.6922816 | 0.6898996 | 0.6870675 | 0.7558344 | 0.7290751 | 1.4548429 | 1.6431706 |
| Phase-1 RCT-15 | 1.3810982 | 1.4762883 | 1.9779413 | 1.6226133 | 1.6030686 | 1.0671923 | 0.9948131 |
| Insulin-like growth factor I | 0.7474025 | 0.5604551 | 0.7700061 | 0.7104682 | 0.6555817 | 0.8579609 | 1.0560609 |
| Sodium/bile acid cotransporter | 0.8168652 | 0.6645965 | 0.9937608 | 0.9386273 | 0.7202144 | 0.7137379 | 0.9612464 |
| Organic cation transporter 3 | 0.8287855 | 0.6814266 | 0.8090365 | 0.9275647 | 0.9031484 | 1.4971648 | 1.5990235 |
| Gadd153 | 1.1358725 | 1.3992534 | 1.098144 | 1.2021248 | 1.3694658 | 1.7199167 | 1.7340665 |
| Phase-1 RCT-109 | 0.7132627 | 0.7022035 | 0.8300777 | 0.7935467 | 0.8679087 | 1.3828288 | 0.9799286 |
| Phase-1 RCT-88 | 1.0365148 | 0.9962185 | 0.923942 | 0.6937807 | 0.9796232 | 0.892311 | 0.8506036 |
| Ubiquitin conjugating enzyme (RAD 6 | 0.9377438 | 0.7298896 | 0.9606111 | 0.9960229 | 0.9553119 | 1.2042702 | 1.4067369 |
| homologue) | | | | | | | |
| Glucose-regulated protein 78 | 0.7816344 | 0.5708965 | 0.790483 | 0.6849909 | 0.6196662 | 2.005804 | 2.0680456 |
| Ribosomal protein L13A | 1.0970287 | 0.7479778 | 0.7215453 | 0.789969 | 0.9818789 | 1.578077 | 1.0621145 |
| Monocyte chemotactic protein receptor (CCR2) | 1.0445595 | 1.0099934 | 0.9158131 | 1.0046194 | 0.9576413 | 1.3135839 | 1.5175824 |
| Fas antigen | 0.9383163 | 0.6881792 | 0.9950752 | 0.9128254 | 0.9539911 | 1.3586603 | 1.3594425 |
| Calpactin I heavy chain | 1.2110449 | 1.5293131 | 1.2360163 | 1.5296632 | 1.3864225 | 0.7915841 | 0.976202 |
| Uncoupling protein 2 | 0.735202 | 0.7342812 | 0.7488037 | 0.8199222 | 0.7870006 | 1.0277493 | 1.021247 |
| Phase-1 RCT-168 | 1.0071242 | 1.0921726 | 1.2083452 | 1.0965936 | 1.075449 | 1.1102885 | 1.1008118 |
| Phase-1 RCT-154 | 1.0556666 | 0.9976771 | 1.2471428 | 1.2551316 | 1.1589812 | 1.8150673 | 1.290509 |
| Superoxide dismutase Mn | 1.1062655 | 1.0694853 | 1.0178945 | 1.0572224 | 1.172151 | 1.3473337 | 1.2469805 |
| Phase-1 RCT-214 | 1.1526017 | 1.0368013 | 1.3128741 | 1.2260255 | 1.1903119 | 0.6174184 | 1.0707477 |
| Phase-1 RCT-225 | 0.8331614 | 0.9656274 | 0.9991004 | 0.8053892 | 0.8582919 | 0.428216 | 0.6079226 |
| Phase-1 RCT-181 | 0.9768243 | 1.1123165 | 0.9355542 | 0.8697043 | 1.0504079 | 0.9068765 | 0.7300206 |
| Nucleoside diphosphate kinase beta | 0.9856877 | 0.91412 | 1.2014649 | 1.2682381 | 1.1269659 | 0.9662601 | 1.3868898 |
| isoform | | | | | | | |
| Extracellular-signal-regulated kinase 1 | 1.4641335 | 0.9969219 | 2.0861804 | 1.4293 | 1.1450347 | 0.6768352 | 0.412362 |
| {Ribosomal protein L6} | 0.869869 | 0.788359 | 1.0216589 | 0.9708834 | 0.881896 | 1.0448707 | 1.1887455 |
| Aldehyde dehydrogenase, microsomal | 0.876411 | 0.897054 | 1.0032759 | 0.9922893 | 1.0209697 | 1.0349674 | 0.9636791 |
| p53 | 0.9827584 | 0.9944749 | 0.9572091 | 0.9318745 | 1.0830653 | 1.303493 | 1.4104387 |
| Phase-1 RCT-239 | 1.2414868 | 1.8535638 | 1.1352313 | 1.6729554 | 1.5435367 | 0.7648112 | 0.5818324 |
| Macrophage inflammatory protein-2 alpha | 0.852841 | 1.0497901 | 0.9137354 | 1.0387785 | 0.948149 | 1.6733148 | 1.5255616 |
| IgE binding protein | 0.8667242 | 0.9929011 | 1.0067452 | 1.0338424 | 1.0572431 | 0.9035317 | 1.1824845 |
| Phase-1 RCT-205 | 1.1246315 | 1.2269077 | 1.2323409 | 1.1318395 | 1.0743102 | 0.9679185 | 0.9607557 |
| Melanoma-associated antigen ME491 | 1.1498673 | 1.0736861 | 1.1964151 | 1.1801275 | 1.1045839 | 0.9681353 | 0.8598748 |
| Phase-1 RCT-242 | 1.1995293 | 1.5524218 | 1.2468494 | 1.3147545 | 1.3025866 | 1.4110525 | 0.9493349 |
| Enolase alpha | 0.6331559 | 0.6330895 | 0.9461446 | 0.9578369 | 0.9278706 | 1.542316 | 2.3143473 |
| Thymosin beta-10 | 0.7237414 | 0.6470189 | 0.735872 | 0.8141183 | 0.7261398 | 0.9112115 | 1.1332175 |
| Cofilin | 1.3694327 | 1.030254 | 1.4801254 | 1.261177 | 1.1888853 | 1.1988536 | 1.059257 |
| Carbonyl reductase | 1.0584143 | 1.2161262 | 0.8222543 | 1.179946 | 1.2975112 | 1.0649179 | 1.188021 |
| MAP kinase kinase | 0.9912357 | 0.95828 | 0.9658388 | 0.9016227 | 0.9070272 | 1.1269094 | 1.2795695 |
| Phase-1 RCT-162 | 0.939432 | 0.9066266 | 1.0321127 | 0.9736348 | 0.8908579 | 1.1634954 | 1.0771184 |
| Phase-1 RCT-72 | 1.0479257 | 1.2596406 | 0.850846 | 1.0079315 | 0.896836 | 0.8830122 | 0.9348046 |
| Ribosomal protein S8 | 0.8184723 | 0.6402165 | 0.9849965 | 0.9056048 | 0.8087254 | 1.3459687 | 1.2631326 |
| Proliferating cell nuclear antigen gene | 1.0946403 | 1.3979219 | 0.992245 | 0.971824 | 0.8865716 | 0.9470069 | 1.961844 |
| Phase-1 RCT-192 | 0.9036466 | 0.8135634 | 1.0137818 | 1.0313817 | 0.9688408 | 1.0884675 | 0.9954352 |
| IkB-a | 0.9153882 | 0.8847584 | 1.0159202 | 1.0271039 | 1.1171277 | 1.197552 | 1.6017346 |
| Endogenous retroviral sequence, 5′ and | 0.771543 | 0.7726386 | 0.8426448 | 0.9285884 | 0.7067065 | 0.7301481 | 0.6492262 |
| 3′ LTR | | | | | | | |
| Phase-1 RCT-37 | 0.9426175 | 0.9016812 | 0.9902634 | 0.9999999 | 0.8586274 | 1.0449872 | 1.0957305 |
| Matrix metalloproteinase-1 | 0.8261954 | 0.6605798 | 0.8004344 | 0.7707496 | 0.816663 | 1.204917 | 1.0540271 |
| Cyclin G | 0.8577724 | 1.3878073 | 1.1336772 | 0.9629484 | 0.8362814 | 2.7899957 | 2.3397868 |
| DNA polymerase beta | 0.983084 | 0.8727085 | 0.9798261 | 0.9944674 | 0.8810854 | 1.0028085 | 1.3320081 |
| Phase-1 RCT-127 | 1.0685972 | 1.1529505 | 0.6706199 | 1.4238687 | 1.3356714 | 0.7725154 | 1.1181643 |
| Multidrug resistant protein-2 | 1.1054993 | 1.2153562 | 0.9508979 | 1.0054368 | 1.0896405 | 3.223368 | 1.3437301 |
| Ribosomal protein S9 | 0.9672169 | 0.7912244 | 1.027324 | 1.0239946 | 1.0765357 | 1.2614537 | 1.5171708 |
| Phase-1 RCT-12 | 1.3823446 | 1.4526114 | 1.4174402 | 1.7869742 | 1.4053019 | 0.9186056 | 0.8884425 |
| Beta-tubulin, class I | 1.9649289 | 1.9077431 | 1.3803464 | 1.6078476 | 1.3034828 | 0.7847885 | 0.8542048 |
| Argininosuccinate lyase | 0.9627877 | 0.9866256 | 1.1904318 | 1.1039255 | 1.1228687 | 1.2225375 | 0.7747435 |
| Elongation factor-1 alpha | 1.0924808 | 0.8293921 | 0.97413 | 1.0194639 | 1.1455259 | 1.1117985 | 1.5073308 |
| Phase-1 RCT-55 | 1.1281741 | 1.3469962 | 1.0710821 | 0.9416949 | 0.9636494 | 1.029152 | 0.7899565 |
| Macrophage inflammatory protein-1 alpha | 1.1149024 | 1.3926147 | 0.8762591 | 0.939153 | 1.0843323 | 1.4332342 | 1.1387316 |
| c-H-ras | 0.7232571 | 0.7521555 | 1.0742891 | 1.0953562 | 1.0156398 | 1.0476233 | 1.6221459 |
| Multidrug resistant protein-1 | 1.3681731 | 1.4882226 | 1.603519 | 1.5283337 | 1.7581807 | 2.2437754 | 1.306301 |
| T-cell cyclophilin | 0.921366 | 0.7874711 | 0.9238024 | 0.8923494 | 0.8854339 | 0.9790981 | 1.3480906 |
| Phase-1 RCT-185 | 1.101825 | 0.8806983 | 1.1570312 | 1.1268035 | 1.0156815 | 0.7479717 | 0.9242129 |
| 25-DX | 0.6784217 | 0.608959 | 0.6903244 | 0.7761578 | 0.7831562 | 1.164624 | 1.4040428 |
| Ref-1 | 0.8855358 | 0.8796749 | 1.146987 | 1.068789 | 1.0085884 | 0.7871952 | 1.2545661 |
| Apolipoprotein CIII | 1.0541718 | 1.034665 | 1.1327796 | 1.2287922 | 1.0853468 | 1.3784631 | 1.386772 |
| NADPH quinone oxidoreductase-1 (DT- | 0.7737947 | 0.7967621 | 1.297991 | 1.0939999 | 0.9122914 | 0.7271929 | 1.3689698 |
| diaphorase) | | | | | | | |
| Pyruvate kinase, muscle | 1.2189789 | 1.7559134 | 0.8935557 | 0.8991864 | 0.8337774 | 0.8189324 | 1.0324525 |
| | ERY | ERY | ERY | ERY | ERY | ERY | EST |
| Compound-Dose (2) | 40 | 40 | 40 | 160 | 160 | 160 | 0.1 |
| Animal Number (3) | 344 | 345 | 346 | 354 | 355 | 356 | 1424 |
| Liver Toxicity Necrosis Classification (4) | no | no | no | no | no | no | no |
| Gene Name (5) | | | | | | | |
| Phase-1 RCT-78 | 1.3046751 | 1.0914379 | 1.0743146 | 1.145496 | 0.9977903 | 0.9885499 | 1.015147 |
| Gamma-actin, cytoplasmic | 0.8171305 | 0.8403273 | 0.5834105 | 0.7089467 | 1 | 0.7684894 | 0.8396725 |
| Matrin F/G | 1.151736 | 0.925684 | 1.0159781 | 1.2375226 | 1.1844898 | 1.2199197 | 1.1184754 |
| Zinc finger protein | 0.5982894 | 0.6860821 | 0.8635595 | 0.9211642 | 0.6985992 | 0.9000989 | 0.9924435 |
| Cathepsin L, sequence 2 | 1.0718793 | 1.3114142 | 1.4819858 | 1.8974347 | 0.7350942 | 1.9309031 | 0.8816093 |
| Gadd45 | 1.2178854 | 0.9731595 | 1.1790946 | 1.032838 | 1.162525 | 0.9564042 | 0.9730492 |
| Phase-1 RCT-144 | 0.8756742 | 0.7410977 | 0.7993088 | 0.8380978 | 0.9137664 | 0.8050461 | 0.964896 |
| Phase-1 RCT-145 | 0.9260749 | 0.8437133 | 0.9145744 | 1.0656359 | 0.6419889 | 0.7902377 | 0.9989576 |
| Phase-1 RCT-50 | 0.5152757 | 0.9588465 | 0.6690646 | 0.7385512 | 0.9553111 | 0.6607592 | 0.7411156 |
| Phase-1 RCT-92 | 1.8560541 | 0.9838004 | 1.4675742 | 1.1654725 | 1.1074867 | 1.3672794 | 0.9541844 |
| Phase-1 RCT-89 | 1.9920813 | 1.5473448 | 2.3659704 | 1.6974288 | 1.2814406 | 1.8845782 | 1.2136217 |
| 14-3-3 zeta | 0.6233349 | 0.7283151 | 0.7665073 | 0.6457143 | 0.7433449 | 0.5301009 | 0.6683149 |
| Dynamin-1 (D100) | 1.925274 | 1.4394305 | 1.7830293 | 1.1512485 | 1.1577648 | 1.4523342 | 1.1063477 |
| Insulin-like growth factor binding protein 1 | 0.9195007 | 1.6170423 | 1.3417126 | 1.4067464 | 0.8920356 | 1.0410556 | 0.9549041 |
| L-gulono-gamma-lactone oxidase | 1.3836896 | 0.7312946 | 1.2617328 | 1.0356843 | 1.5578626 | 0.8594613 | 1.015189 |
| Ornithine decarboxylase | 0.2706858 | 0.3253885 | 0.3865665 | 0.2597117 | 0.493393 | 0.3305529 | 0.6235855 |
| PAR interacting protein | 0.8671227 | 0.7201573 | 0.8708334 | 0.9441991 | 1.0625031 | 0.9904169 | 0.9790523 |
| Phase-1 RCT-128 | 2.9452815 | 1.6105057 | 3.2078798 | 1.411337 | 1.3571572 | 1.7300712 | 1.1151074 |
| Phase-1 RCT-180 | 0.8292764 | 0.697848 | 1.0573006 | 1.3260691 | 1.2723324 | 1.0504124 | 1.1617475 |
| Phase-1 RCT-182 | 1.0213171 | 1.1863085 | 0.858668 | 1.0733824 | 1.0666082 | 1.0488182 | 1.2729136 |
| Phase-1 RCT-207 | 0.7424223 | 0.7127469 | 0.6874069 | 0.8182303 | 0.7082806 | 0.6478244 | 0.8430738 |
| Phase-1 RCT-213 | 0.6974828 | 0.7156252 | 0.7328844 | 0.8738437 | 0.9090805 | 0.8904017 | 0.9192316 |
| Phase-1 RCT-256 | 2.2057862 | 1.4984442 | 2.2053957 | 1.5591079 | 1.6899713 | 1.5582178 | 1.1051437 |
| Phase-1 RCT-258 | 0.8078812 | 0.7461768 | 0.843076 | 0.9282644 | 0.8681315 | 0.6483728 | 0.9988009 |
| Phase-1 RCT-264 | 2.6556458 | 1.8828884 | 3.0006933 | 1.4906067 | 1.7173597 | 1.8781346 | 1.1821638 |
| Phase-1 RCT-271 | 1.5033168 | 0.6939941 | 0.948786 | 0.7513359 | 0.7663074 | 0.7684612 | 0.8402952 |
| Phase-1 RCT-288 | 0.7448587 | 0.7213675 | 0.7222557 | 1.625849 | 0.6033554 | 0.7408314 | 0.7793902 |
| Phase-1 RCT-38 | 2.2436502 | 1.4070039 | 2.276939 | 1.5520036 | 1.6892585 | 1.6098739 | 1.1632563 |
| Phase-1 RCT-39 | 0.7414501 | 1.0818814 | 0.7358919 | 1.165079 | 0.7260877 | 0.882489 | 0.9985314 |
| Phase-1 RCT-68 | 0.9859434 | 0.9328336 | 0.9761741 | 0.9827848 | 1.1401714 | 0.9926258 | 0.9161487 |
| Phase-1 RCT-33 | 1.849916 | 0.8726726 | 1.673844 | 1.1651813 | 1.1581932 | 1.4595691 | 1.1809142 |
| Phase-1 RCT-36 | 1.1959951 | 0.7590257 | 1.1177846 | 0.9616723 | 0.9717681 | 1.1412898 | 1.128536 |
| 17-beta hydroxysteroid dehydrogenase, | 0.7904897 | 0.910664 | 1.2713588 | 0.782394 | 0.5943241 | 0.9492806 | 0.6673186 |
| type 2 | | | | | | | |
| Senescence marker protein-30 | 1.7453419 | 1.9867128 | 2.0194666 | 1.7971244 | 1.4273145 | 1.0728071 | 1.2435108 |
| Ribosomal protein S17 | 1.5942111 | 2.1265278 | 2.0721617 | 1.7979212 | 2.6001446 | 2.111169 | 1.1019536 |
| Phase-1 RCT-83 | 1.0840935 | 1.7109941 | 1.5134729 | 1.3522961 | 0.7312803 | 1.0250876 | 0.877723 |
| Phase-1 RCT-49 | 1.1229011 | 0.7007859 | 0.9866293 | 0.870676 | 0.909496 | 0.8772811 | 0.9544208 |
| Phase-1 RCT-48 | 1.0646036 | 0.6102698 | 0.8151956 | 0.75351 | 1.3863585 | 0.7512516 | 1.0363315 |
| Phase-1 RCT-40 | 1.8156971 | 1.6735761 | 2.2430272 | 1.5040301 | 1.1773834 | 1.3675375 | 0.9525221 |
| Phase-1 RCT-296 | 1.3541422 | 1.2882005 | 1.6139604 | 0.6096316 | 0.7375369 | 0.8171037 | 1.1289173 |
| Phase-1 RCT-295 | 1.0023037 | 0.9455403 | 1.0771772 | 1.0347421 | 1.2947134 | 1.1415977 | 0.9351424 |
| Phase-1 RCT-291 | 0.7963607 | 0.9178221 | 0.7504244 | 0.8135296 | 0.8459877 | 0.9363224 | 1.0686002 |
| Phase-1 RCT-270 | 2.1867974 | 1.2634829 | 1.6172161 | 1.3820782 | 1.4018935 | 1.3082228 | 1.1821332 |
| Phase-1 RCT-241 | 0.802139 | 0.866324 | 0.7138963 | 1.8868235 | 0.7797346 | 1.8610184 | 0.930402 |
| Phase-1 RCT-191 | 0.4825102 | 0.4207209 | 0.5093713 | 0.5588714 | 0.7769824 | 0.6229851 | 0.916745 |
| Phase-1 RCT-189 | 1.7235689 | 1.3627185 | 1.8778596 | 2.1202216 | 1.5402299 | 2.243597 | 1.4367621 |
| Phase-1 RCT-179 | 1.3148415 | 0.7943796 | 0.9626251 | 1.5324435 | 0.4902231 | 1.3260224 | 0.8808916 |
| Phase-1 RCT-152 | 1.6828704 | 2.2160928 | 2.3116677 | 2.0795898 | 2.0914745 | 2.6181242 | 0.9369655 |
| Phase-1 RCT-123 | 0.8516517 | 1.0585556 | 0.7388913 | 0.9148728 | 1.0545751 | 1.0026851 | 0.9629836 |
| Paraoxonase 1 | 1.2883499 | 1.869949 | 1.5313503 | 1.0477068 | 1.1457033 | 1.5317206 | 1.0198061 |
| Organic anion transporter 3 | 0.94003 | 1.2312672 | 0.6946014 | 0.6614157 | 0.6557856 | 0.5875658 | 0.7948477 |
| N-hydroxy-2-acetylaminofluorene | 1.6756074 | 1.8997293 | 2.1344118 | 1.6493942 | 0.9744161 | 1.3544061 | 0.8737929 |
| sulfotransferase (ST1C1) | | | | | | | |
| Integrin beta1 | 0.9654173 | 0.88465 | 1.0476137 | 0.8295673 | 0.8244376 | 0.6180027 | 1.017569 |
| Insulin-like growth factor binding protein 3 | 2.0987406 | 2.2558253 | 2.4470077 | 1.468472 | 1.7170621 | 1.4289383 | 1.3493485 |
| ID-1 | 0.8616903 | 0.7703483 | 0.8531873 | 0.7735415 | 0.8116456 | 0.6086617 | 1.0130625 |
| Hepatic lipase | 1.1511606 | 0.9213136 | 0.9525996 | 0.8697828 | 0.8481318 | 1.017036 | 1.2356069 |
| Heme oxygenase | 1.240772 | 1.6822037 | 1.4343596 | 1.8969213 | 2.1436775 | 2.6557374 | 0.8350033 |
| Equilbrative nitrobenzylthioinosine- | 0.9943014 | 1.2691829 | 1.2500876 | 1.3061357 | 0.745481 | 0.8764174 | 0.8824002 |
| sensitive nucleoside transporter | | | | | | | |
| Epidermal growth factor | 1.4825957 | 1.7736048 | 1.5188228 | 1.3959074 | 1.407102 | 1.4905548 | 1.236545 |
| c-myc | 1.1974717 | 0.878868 | 0.7479487 | 0.9427556 | 0.934615 | 0.8385381 | 0.791741 |
| Carbonic anhydrase III | 1.0248159 | 0.4751967 | 1.0399283 | 0.787604 | 0.4992937 | 0.3743774 | 0.9381163 |
| Beta-actin | 0.7174167 | 0.4646646 | 0.5986167 | 0.6490104 | 0.9104236 | 0.4993384 | 0.7375403 |
| Bax (alpha) | 0.7030144 | 0.6891272 | 0.7565884 | 0.8421159 | 0.864095 | 0.7174688 | 0.9800239 |
| Alpha 1 - inhibitor III | 1.464841 | 1.8750519 | 1.4658511 | 0.9372556 | 0.747706 | 1.4316047 | 1.1347969 |
| 60S ribosomal protein L6 | 1.2947186 | 0.8096044 | 1.3291594 | 1.3582761 | 1.3897492 | 1.3923753 | 0.9137771 |
| Phase-1 RCT-117 | 1.8071829 | 1.6583085 | 1.8017474 | 1.9038724 | 1.2675313 | 2.2720842 | 0.9787938 |
| Phase-1 RCT-102 | 1.6716912 | 0.5363799 | 1.3746179 | 0.5467025 | 0.4971568 | 0.6516236 | 1.1315098 |
| 3-hydroxyisobutyrate dehydrogenase | 1.6447264 | 1.3280048 | 1.7234511 | 1.7089126 | 1.5597272 | 1.696042 | 1.0634521 |
| ATPase inhibitor (rat mitochondrial IF1 | 2.8324342 | 1.2672375 | 3.0221741 | 2.216673 | 1.756264 | 2.9005635 | 1.1742792 |
| protein) | | | | | | | |
| Alpha-2-macroglobulin | 3.4746912 | 0.9778748 | 2.6499164 | 19.667973 | 5.506673 | 5.4624777 | 1.8019046 |
| Phase-1 RCT-137 | 2.3404415 | 1.1240724 | 2.1407337 | 1.6197182 | 2.1235428 | 2.3656025 | 1.2980542 |
| Phase-1 RCT 252 | 1.3956954 | 1.1276152 | 1.4335128 | 0.9871198 | 1.4793962 | 1.1180509 | 1.107401 |
| Phase-1 RCT-65 | 0.7017964 | 0.8908603 | 0.7274052 | 0.8288731 | 0.8946289 | 0.7959291 | 0.7888141 |
| Liver fatty acid binding protein | 2.2449362 | 2.496283 | 3.6212106 | 1.1980082 | 1.1234883 | 1.5622343 | 1.0008434 |
| Carbamyl phosphate synthetase I | 1.494917 | 1.1590773 | 1.5159031 | 0.9578381 | 1.7046676 | 1.0930035 | 1.0471283 |
| c-jun | 0.7979035 | 1.1496426 | 0.9088701 | 0.9409447 | 0.9890342 | 0.8164377 | 0.9429843 |
| Transthyretin | 1.9787489 | 2.320032 | 3.150734 | 1.7089721 | 1.9292778 | 2.1254022 | 1.1740705 |
| Aflatoxin B1 aldehyde reductase | 0.8823617 | 0.5924826 | 0.683098 | 0.8035737 | 0.6150676 | 0.7587878 | 1.2460879 |
| Phase-1 RCT-15 | 0.8967305 | 0.8843715 | 1.0338205 | 0.9235788 | 0.9874468 | 0.947193 | 0.7956206 |
| Insulin-like growth factor I | 2.061099 | 2.1596088 | 2.354777 | 1.4066072 | 1.7076546 | 1.4493039 | 1.352946 |
| Sodium/bile acid cotransporter | 2.9072728 | 1.2932681 | 2.9010882 | 1.2097394 | 0.9115224 | 1.4216633 | 1.0010197 |
| Organic cation transporter 3 | 1.0185238 | 0.9649167 | 1.284659 | 1.336564 | 1.1749829 | 1.3762009 | 0.9740506 |
| Gadd153 | 0.9036861 | 1.0574546 | 1.1207694 | 1.0782987 | 0.8757479 | 0.7575757 | 0.7680007 |
| Phase-1 RCT-109 | 1.4946924 | 0.8285434 | 1.5731704 | 1.1724768 | 1.5357205 | 1.4407775 | 1.1182466 |
| Phase-1 RCT-88 | 1.7924161 | 1.362853 | 1.5018326 | 1.3013394 | 1.4841356 | 1.5591816 | 0.9094634 |
| Ubiquitin conjugating enzyme (RAD6 | 1.1297365 | 0.9944098 | 1.2780479 | 1.304638 | 1.819953 | 1.4897186 | 1.1103649 |
| homologue) | | | | | | | |
| Glucose-regulated protein 78 | 2.2094297 | 2.1173525 | 2.4686866 | 4.4238434 | 1.7441198 | 2.5857375 | 1.0038023 |
| Ribosomal protein L13A | 1.6782147 | 1.0505928 | 2.543329 | 1.4769226 | 1.733376 | 1.5887942 | 1.1009448 |
| Monocyte chemotactic protein receptor (CCR2) | 0.5571817 | 0.8096895 | 0.6004639 | 0.7635297 | 0.873992 | 0.9222661 | 0.795961 |
| Fas antigen | 1.2873343 | 0.865962 | 1.2765297 | 1.4816856 | 1.0371265 | 1.1640005 | 0.8435948 |
| Calpactin I heavy chain | 1.0192211 | 0.7677112 | 1.1755935 | 0.9239366 | 1.0519654 | 0.7994842 | 0.9003265 |
| Uncoupling protein 2 | 0.7763109 | 0.8127323 | 0.9801469 | 0.8549287 | 1.1571794 | 0.9576427 | 1.0614493 |
| Phase-1 RCT-168 | 1.5757682 | 1.0826385 | 1.4992486 | 1.5104128 | 1.3677459 | 1.4286089 | 1.073896 |
| Phase-1 RCT-154 | 1.1190175 | 0.7863556 | 1.0114676 | 1.1182048 | 0.7007902 | 1.1195285 | 0.9322451 |
| Superoxide dismutase Mn | 1.9742026 | 1.7422863 | 1.9135505 | 1.9883803 | 2.6762848 | 1.6509039 | 1.0801138 |
| Phase-1 RCT-214 | 0.6164565 | 0.8168197 | 0.6630925 | 0.9426488 | 0.8484807 | 1.0456159 | 0.897943 |
| Phase-1 RCT-225 | 1.3864579 | 1.2612184 | 0.9234554 | 1.7201121 | 0.9636295 | 0.7713498 | 0.9082298 |
| Phase-1 RCT-181 | 0.91705 | 0.82063 | 0.8395435 | 0.8340051 | 1.0709493 | 0.9943795 | 1.0274212 |
| Nucleoside diphosphate kinase beta | 1.1031327 | 1.0152477 | 1.0638169 | 0.9322887 | 1.3054496 | 1.090767 | 1.2227219 |
| isoform | | | | | | | |
| Extracellular-signal-regulated kinase 1 | 0.6918522 | 0.6397311 | 0.8472724 | 0.7278605 | 0.4875581 | 0.3449439 | 0.9018081 |
| {Ribosomal protein L6} | 1.3267968 | 1.3866663 | 1.5033104 | 1.5365924 | 1.8075202 | 1.6546329 | 1.0230248 |
| Aldehyde dehydrogenase, microsomal | 1.7618445 | 1.1200566 | 1.4895493 | 1.1792433 | 1.3343514 | 1.1736747 | 1.0377368 |
| p53 | 1.6058685 | 1.2048452 | 1.6012113 | 1.5470365 | 1.1115948 | 0.9102609 | 0.8623753 |
| Phase-1 RCT-239 | 0.7917985 | 0.7567933 | 0.7581568 | 0.6466097 | 0.8873342 | 0.7711132 | 0.9150288 |
| Macrophage inflammatory protein-2 alpha | 1.0000918 | 1.076359 | 1.2420377 | 0.9796714 | 1.0720333 | 0.9714794 | 0.9045132 |
| IgE binding protein | 1.2871934 | 1.0357251 | 1.7931908 | 1.2416072 | 1.3345616 | 0.9626111 | 0.9490303 |
| Phase-1 RCT-205 | 1.3263571 | 0.7073438 | 0.9757852 | 1.4601974 | 1.2633361 | 1.5438241 | 1.0698799 |
| Melanoma-associated antigen ME491 | 1.1064799 | 0.8674696 | 1.0899904 | 1.2789402 | 1.3931452 | 1.1017052 | 0.9789908 |
| Phase-1 RCT-242 | 0.6898642 | 0.5412219 | 0.5692167 | 0.8689857 | 0.7949904 | 0.8210483 | 0.9390978 |
| Enolase alpha | 0.9133129 | 1.0887572 | 1.0477728 | 2.1281877 | 2.2828853 | 4.0989804 | 1.3314631 |
| Thymosin beta-10 | 1.4515698 | 1.0113668 | 1.5923477 | 1.4246024 | 1.4631331 | 1.5367264 | 1.2220308 |
| Cofilin | 1.3460637 | 1.2514149 | 1.5662527 | 0.7586643 | 1.3986899 | 1.2835791 | 1.2344989 |
| Carbonyl reductase | 0.6992121 | 0.9555411 | 0.6825498 | 0.7820483 | 0.7315698 | 0.4947433 | 0.8673934 |
| MAP kinase kinase | 0.8533267 | 0.8319414 | 0.9445063 | 0.7824835 | 0.9368709 | 0.4868771 | 1.0518181 |
| Phase-1 RCT-162 | 1.3561025 | 1.5889726 | 1.5252761 | 1.4797984 | 1.8396616 | 1.6578428 | 1.0507632 |
| Phase-1 RCT-72 | 0.7643848 | 1.2055798 | 0.7713375 | 0.880288 | 0.8249179 | 0.6976481 | 0.7828592 |
| Ribosomal protein S8 | 2.0714388 | 2.2812564 | 2.8340156 | 0.8629115 | 3.1145298 | 2.6691117 | 1.0580859 |
| Proliferating cell nuclear antigen gene | 1.2404206 | 1.1262001 | 1.040335 | 0.885608 | 0.966159 | 0.7588708 | 0.8738785 |
| Phase-1 RCT-192 | 0.8644448 | 0.778972 | 0.9775494 | 0.9306437 | 1.0534642 | 1.1018441 | 1.0994567 |
| IkB-a | 0.784856 | 0.573536 | 0.8536878 | 0.8149751 | 1.1345508 | 0.9618451 | 1.4855899 |
| Endogenous retroviral sequence, 5′ and | 1.0465344 | 1.1397475 | 0.9159402 | 1.0911741 | 0.6897357 | 0.5928535 | 0.7465968 |
| 3′ LTR | | | | | | | |
| Phase-1 RCT-37 | 1.0796652 | 1.1470929 | 1.213584 | 1.2654525 | 1.3410114 | 1.1934067 | 0.9148717 |
| Matrix metalloproteinase-1 | 0.9482892 | 0.5328655 | 1.1260147 | 1.0469211 | 1.4483149 | 1.2288436 | 1.2981261 |
| Cyclin G | 0.7886401 | 0.8259432 | 0.7451659 | 0.8877055 | 0.7960341 | 0.750717 | 0.8586254 |
| DNA polymerase beta | 1.2574316 | 1.5501531 | 1.2522966 | 1.022403 | 1.3847826 | 0.9884988 | 1.146003 |
| Phase-1 RCT-127 | 1.2215488 | 1.2673993 | 1.2249299 | 1.184363 | 1.0235173 | 1.478652 | 0.9541326 |
| Multidrug resistant protein-2 | 1.2437081 | 1.348887 | 1.2123268 | 1.0143793 | 1.169789 | 0.8012936 | 0.7831205 |
| Ribosomal protein S9 | 1.2018086 | 1.2504216 | 1.2981393 | 1.3898 | 2.142252 | 1.439242 | 1.0675004 |
| Phase-1 RCT-12 | 0.7140949 | 0.6780132 | 0.807696 | 0.6222308 | 0.8504101 | 0.5237246 | 0.9762024 |
| Beta-tubulin, class I | 0.6582632 | 0.7141493 | 0.8502041 | 0.5189793 | 1.0424433 | 0.6962566 | 1.0109775 |
| Argininosuccinate lyase | 1.0591232 | 1.0438608 | 1.4781908 | 1.0910281 | 1.4374999 | 1.495635 | 1.0888152 |
| Elongation factor-1 alpha | 1.2662908 | 1.2598937 | 1.5798334 | 1.5499823 | 2.0097637 | 1.693962 | 1.1765566 |
| Phase-1 RCT-55 | 0.661125 | 0.9924877 | 0.5247591 | 0.7194569 | 0.6521698 | 0.6378601 | 0.7638164 |
| Macrophage inflammatory protein-1 alpha | 0.541908 | 0.8319678 | 0.5137824 | 0.671406 | 0.8606426 | 0.6907659 | 0.827444 |
| c-H-ras | 0.8271519 | 0.7747231 | 0.9327555 | 1.2355654 | 1.4023541 | 1.2206491 | 0.9227952 |
| Multidrug resistant protein-1 | 0.9630151 | 0.8441162 | 1.1732447 | 1.0608569 | 1.2163931 | 1.0536633 | 0.7654111 |
| T-cell cyclophilin | 1.413001 | 1.4722817 | 1.3778731 | 1.2513797 | 1.578499 | 1.0762656 | 1.1120536 |
| Phase-1 RCT-185 | 0.9475589 | 0.7308424 | 1.0092249 | 0.8517721 | 1.090487 | 1.2458767 | 1.2825836 |
| 25-DX | 2.0025964 | 0.9442398 | 1.8850203 | 1.664301 | 1.4410014 | 1.8977482 | 1.00365 |
| Ref-1 | 2.314484 | 1.2832838 | 2.2737715 | 0.8188228 | 0.8033118 | 1.0195754 | 1.0145404 |
| Apolipoprotein CIII | 0.9925867 | 1.113581 | 1.1459274 | 0.7022407 | 0.7688034 | 0.9297074 | 1.3859916 |
| NADPH quinone oxidoreductase-1 (DT- | 2.2400634 | 1.2531289 | 2.3080895 | 0.8387501 | 0.8309439 | 1.0336238 | 1.0920204 |
| diaphorase) | | | | | | | |
| Pyruvate kinase, muscle | 0.761744 | 1.1551427 | 1.0597123 | 1.0749912 | 1.1219273 | 1.0962286 | 0.847417 |
| | EST | EST | EST | EST | EST | ETH | ETH |
| Compound-Dose (2) | 0.1 | 0.1 | 0.4 | 0.4 | 0.4 | 2500 | 2500 |
| Animal Number (3) | 1425 | 1426 | 1434 | 1435 | 1436 | 134 | 135 |
| Liver Toxicity Necrosis Classification (4) | no | no | no | no | no | no | no |
| Gene Name (5) | | | | | | | |
| Phase-1 RCT-78 | 1.0304314 | 1.1373146 | 0.9370616 | 1.0395666 | 1.0391445 | 0.9164214 | 0.8734835 |
| Gamma-actin, cytoplasmic | 1.2726216 | 0.9196373 | 1.1871346 | 1.2651716 | 1.0286912 | 0.9200018 | 0.9307378 |
| Matrin F/G | 1.2997053 | 1.1500682 | 0.8688852 | 0.9548653 | 0.9332527 | 0.8835666 | 0.7951649 |
| Zinc finger protein | 1.2370325 | 0.9665538 | 0.5840035 | 0.5098187 | 0.5419267 | 0.9035364 | 0.8744882 |
| Cathepsin L, sequence 2 | 1.2463498 | 1.2331133 | 1.1855974 | 1.1856833 | 1.4139824 | 0.8378182 | 0.8895865 |
| Gadd45 | 0.7607668 | 0.8417025 | 0.8753212 | 0.6969673 | 0.8774846 | 1.6427606 | 1.4569082 |
| Phase-1 RCT-144 | 0.8437126 | 0.8762918 | 0.9689837 | 0.9790236 | 1.0122873 | 1.081442 | 1.1252271 |
| Phase-1 RCT-145 | 0.8101653 | 0.82087 | 0.8150347 | 0.7197604 | 0.6864749 | 1.0178725 | 1.1509957 |
| Phase-1 RCT-50 | 0.6195437 | 0.853488 | 0.7776847 | 0.5725228 | 0.7050141 | 1.0673821 | 1.1265911 |
| Phase-1 RCT-92 | 1.0727197 | 1.1371187 | 1.1105787 | 1.5818233 | 1.3493189 | 0.7053197 | 0.7300615 |
| Phase-1 RCT-89 | 1.3255758 | 1.4303848 | 1.1542516 | 1.3531849 | 1.2975395 | 0.8990558 | 0.8448901 |
| 14-3-3 zeta | 0.7597263 | 0.7870657 | 0.6605424 | 0.6036639 | 0.6083623 | 1.236708 | 1.495327 |
| Dynamin-1 (D100) | 1.1622748 | 1.1727291 | 1.1327395 | 1.2956442 | 1.2930012 | 1.052875 | 0.9121175 |
| Insulin-like growth factor binding protein 1 | 1.178661 | 1.0955883 | 1.0447339 | 1.0389254 | 0.9889363 | 1.200666 | 1.1977156 |
| L-gulono-gamma-lactone oxidase | 1.0640885 | 1.4301397 | 1.0640041 | 1.7075694 | 1.367901 | 0.5514012 | 0.4861048 |
| Ornithine decarboxylase | 0.717285 | 0.721472 | 0.6549876 | 0.7772626 | 0.6756911 | 1.1004072 | 1.1450986 |
| PAR interacting protein | 0.956818 | 0.9813302 | 0.9989717 | 0.9886239 | 1.04751 | 1.0705303 | 1.0636709 |
| Phase-1 RCT-128 | 1.294693 | 1.2955294 | 1.2062238 | 1.4110892 | 1.3865926 | 0.9795698 | 0.8117476 |
| Phase-1 RCT-180 | 1.1483281 | 1.0310713 | 0.8550867 | 0.7955197 | 0.7782789 | 0.9105614 | 0.9332984 |
| Phase-1 RCT-182 | 1.2871236 | 1.173113 | 1.2398208 | 1.499539 | 1.2597803 | 0.8650094 | 0.8690648 |
| Phase-1 RCT-207 | 0.7455412 | 0.6722333 | 1.0039043 | 0.9476345 | 0.8913919 | 1.1062521 | 1.2135482 |
| Phase-1 RCT-213 | 0.8470503 | 0.8945488 | 1.0632862 | 0.8746682 | 0.9213482 | 1.0766559 | 1.2222823 |
| Phase-1 RCT-256 | 1.430769 | 1.3862747 | 1.0741428 | 1.1674851 | 1.1853721 | 0.9966137 | 0.7537109 |
| Phase-1 RCT-258 | 0.7695987 | 0.7648709 | 0.7486147 | 0.7130126 | 0.7437233 | 1.0846621 | 1.2426503 |
| Phase-1 RCT-264 | 1.6015104 | 1.7085139 | 2.1963668 | 1.7806599 | 2.1670918 | 0.7562202 | 0.5226375 |
| Phase-1 RCT-271 | 0.7688758 | 0.9026864 | 0.7989568 | 0.9264053 | 0.6186034 | 0.9099602 | 0.7964562 |
| Phase-1 RCT-288 | 1.289207 | 1.2300128 | 1.0914503 | 1.3261905 | 1.5674889 | 0.7834119 | 0.8188883 |
| Phase-1 RCT-38 | 1.5152737 | 1.2743127 | 1.1449655 | 1.3405435 | 1.235193 | 0.7580349 | 0.559616 |
| Phase-1 RCT-39 | 0.8148645 | 0.985646 | 1.0381837 | 0.9632005 | 0.9619061 | 0.9715819 | 1.2165273 |
| Phase-1 RCT-68 | 0.8297506 | 0.904252 | 0.9570798 | 1.0049536 | 1.0097767 | 0.9201565 | 0.8898639 |
| Phase-1 RCT-33 | 1.3452349 | 1.2158774 | 1.339211 | 1.2500211 | 1.2098606 | 0.8918113 | 0.8176999 |
| Phase-1 RCT-36 | 1.1025267 | 0.9779161 | 0.9764539 | 0.8156356 | 0.8033626 | 0.9367272 | 0.940553 |
| 17-beta hydroxysteroid dehydrogenase, | 0.7318453 | 1.3348261 | 0.7074933 | 0.6423734 | 1.0341032 | 0.5708229 | 0.6211091 |
| type 2 | | | | | | | |
| Senescence marker protein-30 | 1.7119398 | 1.8992468 | 1.7557583 | 1.8732315 | 2.0919435 | 0.8742879 | 0.7380418 |
| Ribosomal protein S17 | 1.4899285 | 1.1529039 | 1.3933026 | 1.5315961 | 1.3960164 | 1.3555325 | 1.4826103 |
| Phase-1 RCT-83 | 1.0545739 | 1.0098735 | 1.1746757 | 1.0779393 | 0.9751208 | 1.045696 | 1.051054 |
| Phase-1 RCT-49 | 0.7833854 | 0.8089913 | 0.8498184 | 0.7812656 | 1.0211347 | 1.0137843 | 1.0463966 |
| Phase-1 RCT-48 | 1.1059678 | 1.0457726 | 1.16025 | 1.0438998 | 1.3652995 | 1.1491603 | 0.9107115 |
| Phase-1 RCT-40 | 1.2491119 | 0.9635474 | 1.2794034 | 1.2239301 | 1.1034546 | 0.8871425 | 0.7425576 |
| Phase-1 RCT-296 | 1.4011158 | 1.2786795 | 1.3287432 | 1.1360538 | 1.2712151 | 1.6672184 | 1.0480496 |
| Phase-1 RCT-295 | 1.128038 | 1.0507914 | 1.1473079 | 1.120851 | 1.188442 | 0.9686615 | 0.9391525 |
| Phase-1 RCT-291 | 1.1428862 | 1.0657734 | 0.9839928 | 1.0959567 | 1.169288 | 0.8850941 | 0.8405184 |
| Phase-1 RCT-270 | 1.3803983 | 1.3695438 | 1.3317996 | 1.4330322 | 1.6158919 | 1.0041107 | 1.00365 |
| Phase-1 RCT-241 | 0.8051522 | 0.7855308 | 0.7891998 | 0.7747208 | 0.7262211 | 1.0056971 | 1.1333097 |
| Phase-1 RCT-191 | 0.8075343 | 0.8237102 | 1.0661762 | 1.2965266 | 1.3249499 | 1.079228 | 1.0568122 |
| Phase-1 RCT-189 | 1.5725679 | 1.8467877 | 1.5124946 | 1.3470984 | 1.4619268 | 0.9794118 | 0.7803183 |
| Phase-1 RCT-179 | 1.2538722 | 1.0751095 | 1.0364006 | 1.0424376 | 1.1528999 | 0.8310729 | 0.8527131 |
| Phase-1 RCT-152 | 1.1220673 | 0.9982054 | 1.2110896 | 1.32434 | 1.3338519 | 0.9333929 | 0.9370489 |
| Phase-1 RCT-123 | 0.6993473 | 0.8977193 | 1.275413 | 1.2478033 | 1.3877528 | 1.0723882 | 1.501405 |
| Paraoxonase 1 | 1.5812876 | 1.232887 | 1.4673971 | 1.450182 | 1.7102482 | 0.7966574 | 0.6305997 |
| Organic anion transporter 3 | 0.997758 | 1.1712021 | 0.9505479 | 0.7497862 | 0.7884035 | 1.0547526 | 1.5776743 |
| N-hydroxy-2-acetylaminofluorene | 1.2095144 | 1.1840572 | 1.2011105 | 1.350799 | 1.2607394 | 0.8895048 | 0.8532761 |
| sulfotransferase (ST1C1) | | | | | | | |
| Integrin beta1 | 0.9617203 | 1.2238392 | 0.8861653 | 0.7466592 | 0.7884035 | 1.2805877 | 1.3998353 |
| Insulin-like growth factor binding protein 3 | 1.5020888 | 1.2375914 | 1.8283125 | 1.5511788 | 1.1453799 | 0.9916797 | 0.827675 |
| ID-1 | 0.8746687 | 1 | 0.6578944 | 0.6753635 | 0.673134 | 1.2021545 | 1.6370097 |
| Hepatic lipase | 1.7150329 | 1.0518198 | 1.446564 | 1.2149947 | 1.2364718 | 0.7957267 | 0.7571849 |
| Heme oxygenase | 0.7552453 | 0.8219338 | 1.087707 | 0.7707242 | 0.9808832 | 1.0463618 | 0.8595166 |
| Equilbrative nitrobenzylthioinosine- | 0.9937122 | 1.0826118 | 1.1434855 | 1.1273277 | 1.1259779 | 0.8938875 | 0.9219638 |
| sensitive nucleoside transporter | | | | | | | |
| Epidermal growth factor | 1.0861543 | 1.0888739 | 1.1765313 | 1.101977 | 0.9680227 | 1.1821164 | 1.4139535 |
| c-myc | 0.7705438 | 0.7202084 | 0.7540931 | 0.5315431 | 0.5553087 | 1.3884487 | 1.6744187 |
| Carbonic anhydrase III | 0.937557 | 2.3455582 | 1.1060599 | 2.2234397 | 1.1788235 | 0.6728324 | 0.4177456 |
| Beta-actin | 0.9063423 | 0.7249879 | 0.6726425 | 0.5158747 | 0.5066513 | 1.0311606 | 1.0654813 |
| Bax (alpha) | 0.938217 | 0.7266508 | 0.8932859 | 0.8251526 | 0.8541078 | 1.1723841 | 1.3918605 |
| Alpha 1 - inhibitor III | 1.514807 | 1.1658953 | 1.7412591 | 1.4962704 | 1.1434869 | 0.8339713 | 0.573244 |
| 60S ribosomal protein L6 | 1.171476 | 0.9655044 | 1.1742439 | 0.9645764 | 1.112401 | 1.1117767 | 1.0195934 |
| Phase-1 RCT-117 | 0.975172 | 1.456134 | 1.2112207 | 1.1984391 | 1.2757537 | 0.9608882 | 1.0947727 |
| Phase-1 RCT-102 | 0.8204509 | 1.0402229 | 1.3389049 | 1.0533947 | 1.0439922 | 1.0213945 | 1.003476 |
| 3-hydroxyisobutyrate dehydrogenase | 1.1941687 | 1.3286915 | 1.0816398 | 1.1176853 | 1.2082742 | 0.7321373 | 0.6292353 |
| ATPase inhibitor (rat mitochondrial IF1 | 1.2853584 | 1.2575696 | 1.4711634 | 1.5330365 | 1.5114492 | 0.9120008 | 0.7407866 |
| protein) | | | | | | | |
| Alpha-2-macroglobulin | 1.667525 | 2.0169163 | 1.9843466 | 1.4747986 | 1.0652345 | 1.4017104 | 1.5675918 |
| Phase-1 RCT-137 | 1.6480101 | 1.450947 | 1.4566958 | 1.5378993 | 1.7540562 | 0.8095474 | 0.7889221 |
| Phase-1 RCT 252 | 1.3004028 | 1.3267648 | 1.1294037 | 1.555541 | 1.6444365 | 0.6443826 | 0.5167652 |
| Phase-1 RCT-65 | 0.6928104 | 0.8345931 | 1.0188665 | 0.9032258 | 1.1192377 | 0.9886417 | 0.8596632 |
| Liver fatty acid binding protein | 1.5630232 | 1.1905069 | 2.228143 | 1.5095946 | 1.7093444 | 0.9100733 | 0.6893745 |
| Carbamyl phosphate synthetase I | 1.3790104 | 1.2860732 | 1.0595564 | 1.5190822 | 1.7880905 | 0.6247367 | 0.4812778 |
| c-jun | 0.7676346 | 0.9282296 | 0.7806445 | 0.5450586 | 0.5898693 | 1.316846 | 1.5152702 |
| Transthyretin | 1.5940381 | 1.7494292 | 1.9104134 | 1.3264352 | 1.6158692 | 0.6740241 | 0.4485601 |
| Aflatoxin B1 aldehyde reductase | 1.4205116 | 1.4449948 | 1.3040339 | 1.376069 | 0.8825733 | 0.950681 | 0.6248426 |
| Phase-1 RCT-15 | 1.0149641 | 0.7665772 | 0.9100774 | 0.6818033 | 0.8530754 | 0.9409332 | 0.7392262 |
| Insulin-like growth factor I | 1.778901 | 1.3434197 | 2.1773367 | 1.7058644 | 2.0093734 | 0.9284967 | 0.7512644 |
| Sodium/bile acid cotransporter | 1.6986712 | 1.4382459 | 1.3401401 | 1.389821 | 1.2975508 | 0.5372666 | 0.5477486 |
| Organic cation transporter 3 | 1.0925614 | 1.0572598 | 0.7170525 | 0.6105713 | 0.5946059 | 1.0120239 | 1.003652 |
| Gadd153 | 0.7511739 | 0.828822 | 0.7977565 | 0.839857 | 0.8773079 | 1.34298 | 1.5339345 |
| Phase-1 RCT-109 | 1.319775 | 1.2635435 | 1.209529 | 1.337058 | 1.2669846 | 0.8382108 | 0.9537827 |
| Phase-1 RCT-88 | 1.0466486 | 1.0359122 | 1.0308616 | 1.5352123 | 1.3457224 | 0.8523502 | 0.8932362 |
| Ubiquitin conjugating enzyme (RAD 6 | 1.2518569 | 1.1976771 | 1.110242 | 1.1088881 | 0.9842659 | 1.1255133 | 1.0595036 |
| homologue) | | | | | | | |
| Glucose-regulated protein 78 | 1.3342988 | 1.5195745 | 1.6409583 | 1.632476 | 1.4063464 | 1.1497979 | 1.3315455 |
| Ribosomal protein L13A | 1.334506 | 1.2544535 | 1.4055458 | 1.5514863 | 1.3720982 | 0.974562 | 1.1429586 |
| Monocyte chemotactic protein receptor (CCR2) | 0.8167378 | 0.7730353 | 0.4676351 | 0.439017 | 0.4357051 | 1.1258695 | 1.116279 |
| Fas antigen | 1.0709106 | 1.0974652 | 0.9655132 | 0.6964714 | 0.6039698 | 1.0454319 | 1.117825 |
| Calpactin I heavy chain | 0.7889914 | 0.9139944 | 1.022165 | 1.0197326 | 0.9965501 | 0.9631279 | 1.0223162 |
| Uncoupling protein 2 | 1.0232466 | 1.1592199 | 0.9264468 | 0.6093494 | 0.4797601 | 1.0652039 | 1.3203048 |
| Phase-1 RCT-168 | 1.255812 | 1.2044877 | 1.2159727 | 1.4189221 | 1.3293436 | 0.8953057 | 0.9067386 |
| Phase-1 RCT-154 | 0.8504949 | 0.8730463 | 0.6933155 | 0.7409971 | 0.5378006 | 1.1342084 | 1.2516856 |
| Superoxide dismutase Mn | 1.1902138 | 1.1333333 | 1.2705543 | 1.4292388 | 1.3460727 | 1.3063849 | 1.5980414 |
| Phase-1 RCT-214 | 1.2869326 | 0.8001259 | 1.0555484 | 1.057008 | 0.9803799 | 0.8695967 | 0.6734321 |
| Phase-1 RCT-225 | 0.9618574 | 0.8649966 | 1.0239271 | 1.1455531 | 0.9038883 | 0.8097664 | 1.0183899 |
| Phase-1 RCT-181 | 0.9918725 | 0.9733141 | 1.0694084 | 1.3084567 | 1.310389 | 0.8588813 | 0.9079188 |
| Nucleoside diphosphate kinase beta | 1.4191723 | 1.2129245 | 1.131739 | 1.4120921 | 1.1464012 | 1.0329797 | 1.2023184 |
| isoform | | | | | | | |
| Extracellular-signal-regulated kinase 1 | 0.7717546 | 1.5941315 | 0.8666518 | 0.790318 | 0.6020882 | 0.9122509 | 0.6082891 |
| {Ribosomal protein L6} | 1.2447221 | 0.9935773 | 1.0314767 | 0.9289376 | 1.1132572 | 0.7948974 | 0.6794742 |
| Aldehyde dehydrogenase, microsomal | 1.3795766 | 1.3970591 | 1.2416784 | 1.3841368 | 1.2749565 | 1.0957112 | 1.0308098 |
| p53 | 0.8867909 | 1.0365078 | 1.0248368 | 1.0361072 | 0.7777413 | 1.2521526 | 1.2612504 |
| Phase-1 RCT-239 | 0.8795312 | 0.9927617 | 0.9055252 | 1.1248585 | 1.091172 | 0.680701 | 0.7067862 |
| Macrophage inflammatory protein-2 alpha | 1.008894 | 1.011835 | 0.8961974 | 0.4757374 | 0.6007741 | 1.3143358 | 1.6568985 |
| IgE binding protein | 0.8514996 | 0.8929095 | 0.9083477 | 0.9209281 | 0.8120038 | 0.9000774 | 0.8390552 |
| Phase-1 RCT-205 | 0.8584237 | 0.856757 | 0.9280433 | 0.9778271 | 0.9557968 | 1.169301 | 1.2521225 |
| Melanoma-associated antigen ME491 | 0.9057967 | 1.4374298 | 1.0739312 | 1.0499053 | 1.105317 | 1.0797762 | 1.2323722 |
| Phase-1 RCT-242 | 0.6306937 | 0.7918078 | 0.8002034 | 0.7063569 | 0.7963849 | 1.176859 | 1.3069999 |
| Enolase alpha | 1.2927643 | 1.0842178 | 1.474065 | 1.8991622 | 1.4181077 | 1.6315569 | 1.776944 |
| Thymosin beta-10 | 1.305303 | 1.2993885 | 1.180289 | 1.4917367 | 1.2076551 | 0.8441173 | 0.8799108 |
| Cofilin | 1.5091268 | 1.3984262 | 1.2361985 | 1.1509507 | 1.1028041 | 0.9378684 | 0.6456968 |
| Carbonyl reductase | 0.8795255 | 0.7994532 | 0.7166372 | 0.8377288 | 0.4667849 | 1.2766831 | 1.48294 |
| MAP kinase kinase | 1.0621337 | 1.009124 | 0.999311 | 1.0872375 | 1.0078514 | 1.2043198 | 1.2869639 |
| Phase-1 RCT-162 | 1.2826754 | 1.0709027 | 1.0307014 | 1.0902282 | 1.1327239 | 0.8449562 | 0.8340577 |
| Phase-1 RCT-72 | 0.6231907 | 0.6832535 | 0.9097621 | 0.7989049 | 0.9495032 | 1.0411463 | 1.1040659 |
| Ribosomal protein S8 | 1.5901202 | 1.2074176 | 1.3756219 | 1.5300411 | 1.4453561 | 0.7878938 | 0.7639971 |
| Proliferating cell nuclear antigen gene | 0.6914629 | 0.7713764 | 0.8556057 | 0.8357657 | 0.7837561 | 1.3808198 | 1.499605 |
| Phase-1 RCT-192 | 1.2648046 | 1.1900749 | 1.3014731 | 1.3761735 | 1.5242581 | 1.0709258 | 1.0240375 |
| IkB-a | 1.4280132 | 1.3659623 | 1.4479893 | 1.5759537 | 1.237178 | 1.0275424 | 0.8212305 |
| Endogenous retroviral sequence, 5′ and | 0.7973141 | 0.9291518 | 0.7693273 | 0.8756664 | 0.8849374 | 0.7412376 | 0.7381192 |
| 3′ LTR | | | | | | | |
| Phase-1 RCT-37 | 1.0593352 | 0.9868588 | 0.9729766 | 0.9023704 | 0.9981638 | 0.9250751 | 0.9046563 |
| Matrix metalloproteinase-1 | 1.5453489 | 1.2050109 | 1.0781238 | 1.0077423 | 0.9188837 | 1.0002524 | 1.0101168 |
| Cyclin G | 0.6854 | 0.8179451 | 0.7790716 | 0.7922362 | 0.6680272 | 1.3745749 | 1.6233068 |
| DNA polymerase beta | 1.3494798 | 1.2155513 | 1.309541 | 1.3349464 | 1.0753257 | 1.0084074 | 0.917475 |
| Phase-1 RCT-127 | 0.825873 | 1.0862664 | 0.8033638 | 0.8737323 | 0.8999832 | 0.9441873 | 1.0375093 |
| Multidrug resistant protein-2 | 0.8434491 | 0.9256706 | 1.0008652 | 0.8606504 | 1.0269519 | 1.3461186 | 1.5954124 |
| Ribosomal protein S9 | 1.224928 | 1.0721238 | 1.4660871 | 1.5258466 | 1.2485844 | 1.3009809 | 1.2081486 |
| Phase-1 RCT-12 | 0.9664395 | 0.837669 | 0.8102855 | 0.7788463 | 0.7718306 | 1.0230986 | 1.0833209 |
| Beta-tubulin, class I | 1.2296692 | 0.8636096 | 0.8195305 | 0.7650879 | 0.6129431 | 0.9299687 | 0.926104 |
| Argininosuccinate lyase | 1.4511955 | 1.097112 | 0.8182892 | 0.8788803 | 1.5461082 | 0.7590273 | 0.5735566 |
| Elongation factor-1 alpha | 1.5463958 | 1.2081932 | 1.2515804 | 1.3828574 | 1.561409 | 0.8585613 | 0.6673196 |
| Phase-1 RCT-55 | 0.8514211 | 0.7055094 | 0.9987911 | 0.9634593 | 0.9283994 | 0.9256669 | 0.9973241 |
| Macrophage inflammatory protein-1 alpha | 0.7170748 | 0.6841251 | 0.4503473 | 0.2711811 | 0.3656623 | 3.1567888 | 1.852648 |
| c-H-ras | 0.8465126 | 0.8466879 | 0.8171579 | 0.7996402 | 0.8104363 | 1.1218467 | 1.344779 |
| Multidrug resistant protein-1 | 0.8876729 | 1.1485119 | 0.9263793 | 0.645967 | 1.0715888 | 1.2914727 | 1.4659567 |
| T-cell cyclophilin | 1.5044664 | 1.2310063 | 1.5740523 | 1.6906029 | 1.5992981 | 0.9589102 | 0.8455234 |
| Phase-1 RCT-185 | 1.1385449 | 1.1981014 | 1.0902791 | 1.1392018 | 1.1783406 | 0.7772944 | 0.7243637 |
| 25-DX | 1.4017636 | 1.4651091 | 1.1886983 | 1.3784021 | 1.2833066 | 0.9052278 | 0.8393271 |
| Ref-1 | 0.8650547 | 0.892798 | 1.076828 | 0.9102846 | 0.8998539 | 0.9118171 | 1.0636052 |
| Apolipoprotein CIII | 0.9798673 | 1.3011009 | 1.5328379 | 1.1663746 | 1.4715865 | 1.1037229 | 1.106996 |
| NADPH quinone oxidoreductase-1 (DT- | 0.7618618 | 0.8576011 | 0.7359409 | 0.7183681 | 0.614628 | 0.9087644 | 1.1272768 |
| diaphorase) | | | | | | | |
| Pyruvate kinase, muscle | 0.9097487 | 0.8056371 | 0.8073222 | 0.7705396 | 0.8179476 | 0.8675874 | 0.981047 |
| | ETH | GAN | GAN | GAN | GAN | GAN | GEN |
| Compound-Dose (2) | 2500 | 50 | 50 | 200 | 200 | 200 | 38 |
| Animal Number (3) | 136 | 2444 | 2445 | 2454 | 2455 | 2456 | 224 |
| Liver Toxicity Necrosis Classification (4) | no | no | no | no | no | no | no |
| Gene Name (5) | | | | | | | |
| Phase-1 RCT-78 | 0.9570839 | 0.9401236 | 0.9960887 | 1.0188674 | 0.8192461 | 0.9340264 | 1.0347172 |
| Gamma-actin, cytoplasmic | 0.9078299 | 1.0579581 | 1.0266209 | 0.7731414 | 0.8582206 | 0.9282473 | 1.0249627 |
| Matrin F/G | 0.9347963 | 1.0169727 | 1.0471661 | 1.0407778 | 1.0547374 | 1.264261 | 1.1445736 |
| Zinc finger protein | 0.972632 | 0.9844617 | 1.0190402 | 0.9874076 | 1.036683 | 1.000224 | 0.9904625 |
| Cathepsin L, sequence 2 | 1.1408564 | 1.286843 | 1.3854116 | 1.5707234 | 1.4927233 | 1.3991228 | 1.4296743 |
| Gadd45 | 1.2860966 | 0.9264118 | 1.1269455 | 0.9089785 | 1.2027047 | 1.3163323 | 0.9551437 |
| Phase-1 RCT-144 | 1.1682706 | 0.8925341 | 0.9101061 | 0.8860956 | 0.8989355 | 0.8538638 | 0.9841827 |
| Phase-1 RCT-145 | 1.0647606 | 1.1025668 | 1.0139755 | 1.0128912 | 0.9853479 | 0.9900969 | 0.9663431 |
| Phase-1 RCT-50 | 1.0690888 | 1.0167006 | 0.9806898 | 0.9919552 | 0.9691457 | 0.9564817 | 0.9269477 |
| Phase-1 RCT-92 | 0.5882787 | 1.1623333 | 0.9736474 | 0.9912567 | 1.1284927 | 0.9898719 | 1.0850847 |
| Phase-1 RCT-89 | 0.8618751 | 1.0319623 | 0.9934521 | 0.9320058 | 1.0744749 | 0.9375337 | 1.2107489 |
| 14-3-3 zeta | 1.1589068 | 0.979403 | 0.9817888 | 1.086767 | 0.9546699 | 0.976211 | 1.2061018 |
| Dynamin-1 (D100) | 0.8014669 | 0.9414439 | 0.9981578 | 1.0574175 | 1.0129246 | 0.9600793 | 0.9269477 |
| Insulin-like growth factor binding protein 1 | 1.145748 | 0.9191597 | 0.78057 | 0.9196617 | 0.9237905 | 1.0156784 | 0.9568254 |
| L-gulono-gamma-lactone oxidase | 0.531152 | 1.769623 | 0.6966296 | 1.1602397 | 0.8164291 | 1.1179554 | 1.1271907 |
| Ornithine decarboxylase | 1.0788374 | 1.1985838 | 1.2558339 | 1.0506904 | 0.9498547 | 1.0682747 | 0.9890449 |
| PAR interacting protein | 1.1809511 | 0.9354308 | 0.9772195 | 0.943791 | 0.8984325 | 0.8976926 | 0.9277015 |
| Phase-1 RCT-128 | 0.6787139 | 1.1931058 | 1.2814536 | 1.3669001 | 1.138258 | 1.0752748 | 1.287445 |
| Phase-1 RCT-180 | 0.9509065 | 1.4803859 | 1.0721633 | 0.8750628 | 0.9588732 | 1.128116 | 1.0210711 |
| Phase-1 RCT-182 | 0.921521 | 1.0144898 | 1.1266818 | 1.1011302 | 1.1339732 | 0.9596329 | 0.9475083 |
| Phase-1 RCT-207 | 1.0903504 | 0.9384322 | 0.9862666 | 1.0294182 | 0.9136044 | 0.9230917 | 0.9890449 |
| Phase-1 RCT-213 | 1.1577325 | 0.9597511 | 0.9825435 | 0.9471139 | 0.9526224 | 0.9489765 | 1.0423627 |
| Phase-1 RCT-256 | 0.9368426 | 1.2787114 | 0.8220197 | 0.7984047 | 0.9187123 | 1.0223114 | 1.0767794 |
| Phase-1 RCT-258 | 1.1467519 | 1.0524114 | 1.0686125 | 1.077604 | 1.053211 | 1.0165292 | 1.0093024 |
| Phase-1 RCT-264 | 0.4952682 | 1.4455702 | 1.1750609 | 1.2819909 | 0.8396012 | 0.9839293 | 1.7545687 |
| Phase-1 RCT-271 | 0.7079361 | 1.3668095 | 1.0620906 | 1.0116999 | 0.9160094 | 0.9269968 | 0.8953702 |
| Phase-1 RCT-288 | 0.7954217 | 0.9247562 | 1.216582 | 1.1668884 | 1.4516313 | 1.2300769 | 1.427265 |
| Phase-1 RCT-38 | 0.7196273 | 1.2330629 | 0.8730323 | 0.8456874 | 0.9906432 | 1.1965963 | 1.0782242 |
| Phase-1 RCT-39 | 1.182569 | 0.8658794 | 0.9589824 | 1.0253248 | 0.957026 | 0.9618945 | 0.9752221 |
| Phase-1 RCT-68 | 0.9390233 | 0.9655551 | 1.1000869 | 1.0038714 | 1.0417237 | 1.0134537 | 0.9698731 |
| Phase-1 RCT-33 | 0.818787 | 1.7565603 | 1.1223489 | 1.2900771 | 1.1409335 | 1.0846313 | 1.0783039 |
| Phase-1 RCT-36 | 0.9178355 | 1.1024232 | 0.8833934 | 0.9799833 | 0.962268 | 0.9945375 | 0.9138178 |
| 17-beta hydroxysteroid dehydrogenase, | 0.5364773 | 0.9410726 | 1.0604885 | 0.9038597 | 0.6390444 | 1.1015714 | 0.7955377 |
| type 2 | | | | | | | |
| Senescence marker protein-30 | 0.755304 | 0.9124727 | 0.7882803 | 1.2741297 | 1.0138576 | 1.2730807 | 1.6797433 |
| Ribosomal protein S17 | 1.4480318 | 1.2826499 | 1.1597878 | 1.290055 | 1.5469812 | 1.5309917 | 1.6488748 |
| Phase-1 RCT-83 | 1.0150052 | 0.7215759 | 1.1317664 | 1.0989369 | 0.8062131 | 0.8345913 | 1.1775727 |
| Phase-1 RCT-49 | 1.1178201 | 1.002063 | 0.9867035 | 1.0544252 | 0.9888685 | 1.0253555 | 0.8995045 |
| Phase-1 RCT-48 | 0.9299795 | 0.9271559 | 0.9909442 | 1.1088219 | 0.9067153 | 0.8599532 | 1.2042587 |
| Phase-1 RCT-40 | 0.7913646 | 0.8231138 | 1.1422206 | 1.3273443 | 1.0629424 | 1.1622511 | 1.6212785 |
| Phase-1 RCT-296 | 0.7264944 | 1.863238 | 0.9543105 | 1.0440664 | 1.4472808 | 1.451357 | 1.3738534 |
| Phase-1 RCT-295 | 0.8606697 | 1.3871534 | 1.1189808 | 1.1931505 | 1.1257223 | 1.1046046 | 1.2459085 |
| Phase-1 RCT-291 | 0.8500833 | 1.1910933 | 1.0468112 | 1.0232352 | 1.0851074 | 1.0645692 | 1.0854456 |
| Phase-1 RCT-270 | 1.0005586 | 1.0828471 | 1.3685873 | 1.4785362 | 1.2528906 | 1.2349112 | 1.1232624 |
| Phase-1 RCT-241 | 1.2806469 | 1.0485976 | 1.1292498 | 0.9629161 | 1.0860603 | 1.0486856 | 0.9333586 |
| Phase-1 RCT-191 | 1.1078466 | 1.0070107 | 0.9413266 | 0.8729432 | 0.820135 | 0.891997 | 0.9401304 |
| Phase-1 RCT-189 | 1.0318937 | 1.2396235 | 1.3583872 | 1.1041511 | 0.9503825 | 0.8520409 | 1.3046457 |
| Phase-1 RCT-179 | 0.8471132 | 0.9974559 | 1.1154954 | 1.0404768 | 1.0715814 | 0.9736068 | 1.1197009 |
| Phase-1 RCT-152 | 0.939613 | 0.988084 | 1.0349246 | 1.0153865 | 1.2406565 | 1.0663793 | 1.0407035 |
| Phase-1 RCT-123 | 1.057038 | 1.1351519 | 1.1204118 | 1.0252614 | 0.9466922 | 0.9805035 | 0.9327767 |
| Paraoxonase 1 | 0.652566 | 1.0119269 | 1.0515544 | 1.0126017 | 0.9985652 | 0.9427155 | 1.2265178 |
| Organic anion transporter 3 | 1.0282512 | 1.1180246 | 0.9492819 | 0.9730621 | 0.9489927 | 0.9470705 | 1.0033443 |
| N-hydroxy-2-acetylaminofluorene | 0.7689211 | 0.7555214 | 1.3643974 | 1.2059019 | 0.7605674 | 0.7477419 | 1.3029667 |
| sulfotransferase (ST1C1) | | | | | | | |
| Integrin beta1 | 1.2393625 | 1.086583 | 0.9676085 | 1.0407782 | 1.0615511 | 1.1349196 | 0.9612404 |
| Insulin-like growth factor binding protein 3 | 0.9386647 | 0.8592721 | 0.8777552 | 1.2081157 | 1.334101 | 1.1207776 | 1.1160375 |
| ID-1 | 1.2706339 | 0.8444542 | 0.923841 | 0.8809629 | 0.8706906 | 0.8577257 | 0.8916695 |
| Hepatic lipase | 0.53732 | 1.1703931 | 0.5905507 | 0.6695718 | 0.6638333 | 0.6806656 | 1.2096354 |
| Heme oxygenase | 0.9338662 | 1.0543134 | 1.4806718 | 1.0965084 | 1.1825075 | 1.1775445 | 0.9275899 |
| Equilbrative nitrobenzylthioinosine- | 0.8331869 | 0.7312816 | 1.085001 | 0.9602932 | 0.7225043 | 0.7499293 | 1.0579885 |
| sensitive nucleoside transporter | | | | | | | |
| Epidermal growth factor | 1.1419697 | 0.98602 | 1.1329082 | 0.9790036 | 1.129542 | 0.9915487 | 0.9677478 |
| c-myc | 1.4409946 | 0.8655587 | 0.9603919 | 0.9592961 | 1.0782863 | 1.0097561 | 1.0161318 |
| Carbonic anhydrase III | 0.2428967 | 0.3889415 | 1.088523 | 1.3210369 | 0.4628311 | 0.7019867 | 1.642343 |
| Beta-actin | 1.1502745 | 1.081891 | 0.9214643 | 0.907752 | 0.7193065 | 0.7630227 | 1.2780128 |
| Bax (alpha) | 1.2297782 | 0.7397447 | 0.8250983 | 0.7300965 | 0.7553867 | 0.7800917 | 0.9297409 |
| Alpha 1 - inhibitor III | 0.4394174 | 1.0590283 | 1.034861 | 1.1419841 | 0.8085473 | 0.8408454 | 1.0951537 |
| 60S ribosomal protein L6 | 1.0304277 | 1.4700705 | 1.4022598 | 1.4838413 | 1.3889477 | 1.4378998 | 1.010515 |
| Phase-1 RCT-117 | 0.8503636 | 1.0330738 | 1.0984658 | 1.1961775 | 1.216433 | 1.3383827 | 0.9856468 |
| Phase-1 RCT-102 | 0.7765626 | 0.9699659 | 0.5255153 | 0.8580277 | 0.7780008 | 0.7410366 | 1.0219659 |
| 3-hydroxyisobutyrate dehydrogenase | 0.5383043 | 1.2610571 | 1.3318844 | 1.2482458 | 1.2904385 | 1.2378464 | 1.2376674 |
| ATPase inhibitor (rat mitochondrial IF1 | 0.5949401 | 0.8332637 | 1.254381 | 1.0858027 | 1.0897853 | 0.859296 | 2.188757 |
| protein) | | | | | | | |
| Alpha-2-macroglobulin | 2.2345076 | 0.8334745 | 1.0073193 | 1.1459711 | 1.0057951 | 1.325619 | 1.2041867 |
| Phase-1 RCT-137 | 0.552071 | 1.2764891 | 1.0954912 | 0.8878304 | 0.9442198 | 0.9133599 | 1.4234064 |
| Phase-1 RCT 252 | 0.5571204 | 1.4427983 | 1.2419162 | 0.8935595 | 1.1765254 | 0.8771051 | 1.1809505 |
| Phase-1 RCT-65 | 0.9626862 | 1.0422832 | 0.9805102 | 1.0602915 | 1.0635049 | 1.1311435 | 1.0710588 |
| Liver fatty acid binding protein | 0.635787 | 1.71954 | 0.8121474 | 1.0035266 | 1.1018152 | 1.2908162 | 3.1550813 |
| Carbamyl phosphate synthetase I | 0.5469739 | 1.4817657 | 1.2584076 | 0.970985 | 1.3614185 | 0.9931091 | 1.266494 |
| c-jun | 1.3537251 | 0.9039223 | 0.8547638 | 0.8765785 | 0.9024624 | 1.1536993 | 0.9377534 |
| Transthyretin | 0.4162147 | 1.0997636 | 0.8990679 | 1.0648751 | 0.7772953 | 0.8448787 | 1.5377084 |
| Aflatoxin B1 aldehyde reductase | 0.5731229 | 0.8952044 | 0.8770782 | 0.8885158 | 0.8413752 | 0.7975582 | 0.874931 |
| Phase-1 RCT-15 | 0.8086339 | 1.1443678 | 1.2096839 | 0.9571105 | 0.9622878 | 1.0959152 | 1.0560554 |
| Insulin-like growth factor I | 0.7335148 | 1.0911248 | 0.7796723 | 1.0827926 | 1.3055578 | 1.0789046 | 1.4983151 |
| Sodium/bile acid cotransporter | 0.5622697 | 1.0388852 | 0.9110429 | 0.9646363 | 0.9078708 | 0.7180137 | 1.0354079 |
| Organic cation transporter 3 | 1.037116 | 1.0330167 | 1.0868998 | 1.1240776 | 1.231008 | 1.104889 | 0.9458486 |
| Gadd153 | 1.2632084 | 0.746016 | 0.8571322 | 0.8000311 | 0.8743864 | 0.8406588 | 0.9469438 |
| Phase-1 RCT-109 | 0.949526 | 1.1794771 | 1.0773382 | 1.2561204 | 1.3256557 | 1.2162268 | 1.283403 |
| Phase-1 RCT-88 | 0.7324827 | 1.0434328 | 0.9986464 | 1.16451 | 1.0642401 | 1.0492612 | 1.0712706 |
| Ubiquitin conjugating enzyme (RAD 6 | 0.9343724 | 1.0099261 | 1.0030762 | 1.0932103 | 1.2789837 | 1.278054 | 1.1439824 |
| homologue) | | | | | | | |
| Glucose-regulated protein 78 | 1.2719611 | 1.0131451 | 0.9472271 | 0.8703954 | 1.0328139 | 0.9787323 | 1.7642215 |
| Ribosomal protein L13A | 1.105641 | 1.0137618 | 0.8964157 | 1.2243375 | 1.3325597 | 1.2555847 | 1.4471568 |
| Monocyte chemotactic protein receptor (CCR2) | 1.0635276 | 0.9999543 | 1.0318146 | 0.9496133 | 1.1387159 | 1.0802208 | 0.9218689 |
| Fas antigen | 1.1851271 | 0.9025369 | 0.9621458 | 0.9836058 | 0.8986581 | 1.0111462 | 1.0369841 |
| Calpactin I heavy chain | 1.0810969 | 1.0015385 | 1.0217333 | 1.0410714 | 1.1729668 | 1.1133566 | 0.9291288 |
| Uncoupling protein 2 | 1.1257765 | 1.1632596 | 0.9457365 | 0.8681848 | 0.9191675 | 0.8793616 | 1.2037797 |
| Phase-1 RCT-168 | 1.1216338 | 1.1330884 | 1.0570587 | 1.0595481 | 0.9836004 | 0.9966976 | 0.922856 |
| Phase-1 RCT-154 | 1.070859 | 1.1688327 | 1.1167089 | 0.8768376 | 1.1130904 | 1.0912505 | 0.9974479 |
| Superoxide dismutase Mn | 1.3302705 | 0.9403153 | 0.9566949 | 1.0175234 | 1.0968761 | 0.9910877 | 1.0768795 |
| Phase-1 RCT-214 | 0.6535299 | 1.1372943 | 1.0195416 | 0.852805 | 0.9330494 | 1.0174085 | 1.0551077 |
| Phase-1 RCT-225 | 0.8545186 | 0.8345152 | 0.8050914 | 0.899617 | 0.8663164 | 0.9047832 | 0.929286 |
| Phase-1 RCT-181 | 0.8274484 | 1.2502651 | 1.041412 | 1.0044295 | 1.1977609 | 1.3564018 | 1.0679021 |
| Nucleoside diphosphate kinase beta | 0.8978298 | 1.1134032 | 1.0292487 | 1.0950992 | 1.5023603 | 1.2590969 | 1.1019003 |
| isoform | | | | | | | |
| Extracellular-signal-regulated kinase 1 | 0.590655 | 0.7095702 | 1.0301657 | 0.9267958 | 0.7216595 | 0.8558136 | 1.0212431 |
| {Ribosomal protein L6} | 0.7867172 | 1.3107276 | 1.1808417 | 1.3384054 | 1.3609494 | 1.2922467 | 1.1320393 |
| Aldehyde dehydrogenase, microsomal | 0.9797402 | 1.1008818 | 1.0408959 | 1.1359932 | 1.1262974 | 1.1957495 | 1.1792107 |
| p53 | 0.9461566 | 0.9716878 | 1.0376903 | 0.956326 | 0.9459599 | 0.9700699 | 0.9504055 |
| Phase-1 RCT-239 | 0.8583314 | 0.9261603 | 0.8891352 | 1.01074 | 0.9126832 | 0.937862 | 0.9358987 |
| Macrophage inflammatory protein-2 alpha | 1.2833334 | 1.005572 | 1.5816623 | 0.9999999 | 1.1279119 | 1.0858263 | 0.9224855 |
| IgE binding protein | 0.8977981 | 1.0465435 | 1.1453815 | 1.1530309 | 1.1703004 | 1.142045 | 0.8116588 |
| Phase-1 RCT-205 | 1.1805941 | 1.0316281 | 1.0532663 | 1.0355963 | 1.0139332 | 0.9347453 | 0.9123045 |
| Melanoma-associated antigen ME491 | 1.1641998 | 0.89261 | 1.1140764 | 1.2352614 | 0.8724201 | 0.8399391 | 0.9570356 |
| Phase-1 RCT-242 | 1.1310357 | 0.9102228 | 0.9558458 | 0.8615268 | 0.8274908 | 1.102919 | 0.8944477 |
| Enolase alpha | 1.5838566 | 1.1487687 | 1.2240095 | 0.8896188 | 0.9893223 | 0.7948512 | 0.7688351 |
| Thymosin beta-10 | 0.8007828 | 1.0226659 | 0.9214841 | 1.0081626 | 1.0814888 | 1.0255475 | 1.5219233 |
| Cofilin | 0.7412532 | 1.1032263 | 1.0245838 | 1.1768026 | 1.0187398 | 1.0796642 | 1.1997886 |
| Carbonyl reductase | 1.1874046 | 1.1463575 | 0.9205086 | 0.7950811 | 0.906508 | 0.8447568 | 0.8939434 |
| MAP kinase kinase | 1.3002908 | 0.8221036 | 0.8605584 | 0.8396576 | 0.9363269 | 0.9195095 | 0.9737998 |
| Phase-1 RCT-162 | 0.7613506 | 1.1150295 | 1.1095245 | 1.1246326 | 1.1930673 | 1.0812286 | 1.0404065 |
| Phase-1 RCT-72 | 1.0457451 | 1.0213201 | 1.0129923 | 0.9477841 | 0.9273486 | 1.0046018 | 1.0128772 |
| Ribosomal protein S8 | 0.666773 | 1.4165472 | 1.301106 | 1.4372195 | 1.6588395 | 1.6420211 | 1.3388916 |
| Proliferating cell nuclear antigen gene | 1.2244556 | 0.7595564 | 0.8328493 | 0.7557602 | 0.850297 | 0.7876923 | 1.5475842 |
| Phase-1 RCT-192 | 1.0194354 | 1.1065124 | 0.9881312 | 1.0662551 | 1.3071494 | 1.2148783 | 1.1877639 |
| IkB-a | 0.8844844 | 0.891565 | 0.8953676 | 0.7877986 | 0.8725718 | 0.7947372 | 1.0274842 |
| Endogenous retroviral sequence, 5′ and | 0.726558 | 0.722918 | 0.7864444 | 1.307152 | 0.7023762 | 0.8040537 | 1.3956914 |
| 3′ LTR | | | | | | | |
| Phase-1 RCT-37 | 0.8964241 | 1.2269347 | 1.1489819 | 1.1280755 | 1.185578 | 1.1031748 | 0.9479765 |
| Matrix metalloproteinase-1 | 0.9255856 | 0.9420863 | 1.0203842 | 1.0807967 | 1.2658478 | 1.023542 | 1.1683971 |
| Cyclin G | 1.2835743 | 0.9119277 | 0.9947406 | 0.9805602 | 0.97022 | 0.9889929 | 0.9067066 |
| DNA polymerase beta | 0.7597768 | 1.0838422 | 1.0825723 | 1.001945 | 1.1445652 | 1.1796546 | 0.9746843 |
| Phase-1 RCT-127 | 0.8790295 | 1.0247047 | 1.0557346 | 1.1815773 | 1.1086482 | 1.1420695 | 0.923994 |
| Multidrug resistant protein-2 | 1.5065068 | 0.9127318 | 0.9793311 | 1.0613372 | 1.1141677 | 1.0691438 | 0.9155075 |
| Ribosomal protein S9 | 0.8816395 | 0.8975217 | 0.9496523 | 0.8582842 | 1.1073796 | 1.137837 | 1.5202748 |
| Phase-1 RCT-12 | 1.0719265 | 0.905771 | 0.8567398 | 0.9113789 | 0.8819015 | 0.8992224 | 0.9284709 |
| Beta-tubulin, class I | 0.9484149 | 1.2590909 | 0.7139844 | 0.940439 | 0.9863808 | 1.0336927 | 1.1051316 |
| Argininosuccinate lyase | 0.5314547 | 1.384146 | 1.3494147 | 1.2397845 | 1.7201061 | 1.1644546 | 1.1223149 |
| Elongation factor-1 alpha | 0.9045184 | 1.065813 | 1.030179 | 1.4024352 | 1.2551086 | 1.1639271 | 1.2484771 |
| Phase-1 RCT-55 | 0.9382 | 0.9075671 | 0.8891872 | 0.8127232 | 0.8456062 | 0.8768845 | 1.0245502 |
| Macrophage inflammatory protein-1 alpha | 1.3597641 | 0.9348777 | 1.0203453 | 0.9560574 | 0.9428449 | 0.911687 | 1.0122651 |
| c-H-ras | 1.126374 | 0.7976978 | 1.3341768 | 1.1276052 | 1.1260784 | 1.1889749 | 1.3012285 |
| Multidrug resistant protein-1 | 1.3232402 | 1.0387156 | 1.0215632 | 0.9612642 | 1.0127537 | 1.0274177 | 0.9631196 |
| T-cell cyclophilin | 0.6362821 | 1.2082281 | 1.0765046 | 1.1103425 | 1.2069904 | 1.2437195 | 1.4995655 |
| Phase-1 RCT-185 | 0.5549018 | 1.0505164 | 1.2550198 | 1.0576522 | 0.9334025 | 0.8813663 | 1.0844964 |
| 25-DX | 0.8444488 | 0.8307657 | 0.9244376 | 1.1979418 | 0.9428057 | 1.0496641 | 1.371578 |
| Ref-1 | 0.8875001 | 1.0749724 | 1.0133383 | 0.928237 | 0.9607235 | 0.9963501 | 0.9191964 |
| Apolipoprotein CIII | 0.8830666 | 1.0024836 | 1.0082632 | 1.1322832 | 0.9541856 | 0.8737479 | 1.0402081 |
| NADPH quinone oxidoreductase-1 (DT- | 0.8069846 | 1.216832 | 1.0020539 | 0.9445918 | 1.2794201 | 1.1562097 | 0.9344609 |
| diaphorase) | | | | | | | |
| Pyruvate kinase, muscle | 1.1860474 | 0.9981166 | 0.991918 | 0.8848587 | 0.8522423 | 0.8364939 | 0.9217162 |
| | GEN | GEN | GEN | GEN | GEN | HYD | HYD |
| Compound-Dose (2) | 38 | 38 | 150 | 150 | 150 | 250 | 250 |
| Animal Number (3) | 225 | 226 | 234 | 235 | 236 | 1224 | 1225 |
| Liver Toxicity Necrosis Classification (4) | no | no | no | no | no | no | no |
| Gene Name (5) | | | | | | | |
| Phase-1 RCT-78 | 1.055189 | 1.2747266 | 0.9352415 | 1.0513656 | 1.2005216 | 1.0661025 | 1.0353534 |
| Gamma-actin, cytoplasmic | 0.8198518 | 0.6198857 | 1.2702836 | 0.6898923 | 0.9363558 | 0.7868741 | 0.9302737 |
| Matrin F/G | 0.7521346 | 0.7571958 | 1.0079609 | 0.9293442 | 1.096076 | 0.9130963 | 0.7689677 |
| Zinc finger protein | 1.1557597 | 1.0622607 | 0.9375411 | 1.285257 | 1.1806573 | 1.1412085 | 1.0519023 |
| Cathepsin L, sequence 2 | 1.1572617 | 1.2643687 | 1.3700572 | 1.3322263 | 1.4139736 | 0.9163396 | 0.7124679 |
| Gadd45 | 0.8705422 | 0.9115949 | 1.0389977 | 0.9699194 | 0.9641112 | 0.8295172 | 1.0562928 |
| Phase-1 RCT-144 | 1.042422 | 0.885382 | 0.8390424 | 0.8312341 | 0.8745766 | 0.9861053 | 1.0557332 |
| Phase-1 RCT-145 | 0.9356991 | 0.9614306 | 0.747204 | 0.9580887 | 0.911815 | 0.9063122 | 1.0724459 |
| Phase-1 RCT-50 | 1.0640572 | 1.188228 | 1.0060835 | 0.9323747 | 0.8741948 | 1.2220888 | 1.1046655 |
| Phase-1 RCT-92 | 0.7379317 | 0.9804677 | 1.0863873 | 1.3362879 | 1.1286887 | 1.0517061 | 1.1688839 |
| Phase-1 RCT-89 | 1.099622 | 1.1102651 | 0.9022881 | 1.1569375 | 1.0637238 | 1.0269579 | 0.9988757 |
| 14-3-3 zeta | 1.2623328 | 1.1191938 | 0.8584284 | 1.1781572 | 1.1074169 | 0.8714129 | 0.922316 |
| Dynamin-1 (D100) | 0.925337 | 1.040784 | 1.0976964 | 1.12729 | 1.0962949 | 1.0989608 | 1.0874774 |
| Insulin-like growth factor binding protein 1 | 1.0825399 | 1.1700188 | 1.4345232 | 2.0921576 | 1.605083 | 1.0410452 | 0.9582354 |
| L-gulono-gamma-lactone oxidase | 0.7047218 | 0.6478616 | 0.9738993 | 0.7985683 | 0.9590141 | 0.9905555 | 1.0557543 |
| Ornithine decarboxylase | 0.9453914 | 1.0401229 | 1.1253372 | 1.16706 | 1.071773 | 0.7638575 | 0.9981266 |
| PAR interacting protein | 0.9102975 | 0.8165684 | 0.7777603 | 0.8567944 | 0.9600295 | 0.8471083 | 1.0028222 |
| Phase-1 RCT-128 | 0.9570573 | 1.0700732 | 1.1650583 | 1.1986556 | 1.126991 | 0.9878134 | 0.9231682 |
| Phase-1 RCT-180 | 0.6544365 | 0.7555676 | 0.938089 | 0.9675677 | 1.0151885 | 0.9736103 | 1.2165886 |
| Phase-1 RCT-182 | 0.8605765 | 0.9579595 | 0.8972058 | 0.9338855 | 1.0664732 | 0.8882381 | 0.9195814 |
| Phase-1 RCT-207 | 1.0235428 | 0.9624717 | 0.6960295 | 0.9774832 | 0.9121956 | 0.9298088 | 1.0920497 |
| Phase-1 RCT-213 | 0.928848 | 0.8401216 | 0.7506663 | 0.9507741 | 0.9438039 | 0.9595467 | 0.9995154 |
| Phase-1 RCT-256 | 0.7391604 | 0.7439596 | 0.988822 | 0.9327653 | 0.8714823 | 0.9307904 | 1.0040214 |
| Phase-1 RCT-258 | 0.8853754 | 0.8421255 | 0.7389643 | 0.8995013 | 0.8985091 | 0.9219119 | 0.9441162 |
| Phase-1 RCT-264 | 1.1848342 | 1.2938552 | 1.1302369 | 1.7192897 | 1.5427473 | 0.8440461 | 0.9338263 |
| Phase-1 RCT-271 | 0.5411952 | 0.6070189 | 0.8597602 | 0.7572667 | 0.5278856 | 0.7633346 | 0.8477693 |
| Phase-1 RCT-288 | 1.2891071 | 1.4345571 | 1.1376855 | 1.7075455 | 1.4425079 | 0.9810932 | 1.0711797 |
| Phase-1 RCT-38 | 0.7590243 | 0.7487763 | 1.0200139 | 0.8666822 | 0.824148 | 0.9276527 | 0.9867327 |
| Phase-1 RCT-39 | 1.084732 | 1.0781833 | 0.9069771 | 0.7637644 | 0.8367725 | 0.9581429 | 0.8392383 |
| Phase-1 RCT-68 | 1.2212994 | 1.2813135 | 1.2087077 | 1.0305592 | 1.279018 | 0.9820305 | 1.0912864 |
| Phase-1 RCT-33 | 0.8468769 | 0.7416762 | 1.1751765 | 0.7837893 | 0.9402119 | 0.8389993 | 0.9821137 |
| Phase-1 RCT-36 | 0.9046669 | 0.7820277 | 1.1100166 | 0.8109469 | 0.926394 | 0.9665701 | 1.087737 |
| 17-beta hydroxysteroid dehydrogenase, | 1.0122778 | 1.4098544 | 0.8313661 | 1.0563204 | 0.3824679 | 0.6028804 | 0.364169 |
| type 2 | | | | | | | |
| Senescence marker protein-30 | 1.3482227 | 1.1890293 | 1.244297 | 1.388354 | 1.2558265 | 0.8405275 | 0.9360676 |
| Ribosomal protein S17 | 1.0885835 | 1.1023833 | 1.5467294 | 1.3189099 | 1.2300284 | 0.8983638 | 0.8893859 |
| Phase-1 RCT-83 | 1.2326087 | 1.3073136 | 0.888756 | 1.3042663 | 0.9282577 | 1.1600358 | 1.0628606 |
| Phase-1 RCT-49 | 1.0603086 | 0.9914631 | 0.9035108 | 0.9641152 | 0.9724265 | 1.0693439 | 0.9832966 |
| Phase-1 RCT-48 | 1.1745371 | 0.9622536 | 1.6593747 | 1.8654797 | 1.3337924 | 1.1499543 | 1.4814893 |
| Phase-1 RCT-40 | 1.125579 | 1.1318979 | 1.1579304 | 1.4880637 | 1.2114936 | 0.9542326 | 0.9344972 |
| Phase-1 RCT-296 | 1.0843495 | 0.792425 | 1.2385194 | 1.604609 | 0.8761265 | 0.9452577 | 0.8324739 |
| Phase-1 RCT-295 | 1.0558313 | 1.0706142 | 1.3086264 | 1.06439 | 1.0559859 | 0.8401974 | 0.9426808 |
| Phase-1 RCT-291 | 0.7223049 | 0.7372149 | 0.9854612 | 0.8459615 | 0.9915227 | 0.9698417 | 0.9240737 |
| Phase-1 RCT-270 | 0.934988 | 1.1213268 | 0.9305639 | 1.0399462 | 1.1381116 | 1.0113083 | 1.1271573 |
| Phase-1 RCT-241 | 0.8974428 | 0.7500152 | 0.7268621 | 0.8484215 | 0.9124418 | 1.192604 | 1.1218424 |
| Phase-1 RCT-191 | 0.9251101 | 0.8495165 | 0.9176829 | 0.9168581 | 0.9582346 | 1.119819 | 1.2205441 |
| Phase-1 RCT-189 | 0.9279706 | 1.0938208 | 0.9481671 | 1.1903716 | 1.0813708 | 0.9955143 | 1.0355072 |
| Phase-1 RCT-179 | 1.1773332 | 1.1081388 | 1.1151202 | 1.2735337 | 1.1303053 | 0.8986561 | 0.8561955 |
| Phase-1 RCT-152 | 0.9361963 | 0.9521914 | 1.1898364 | 0.9580758 | 0.9925607 | 0.8629715 | 0.8372306 |
| Phase-1 RCT-123 | 1.0334674 | 1.0089153 | 0.9840272 | 0.9524226 | 0.8834574 | 1.1530699 | 1.1157596 |
| Paraoxonase 1 | 0.9521239 | 1.0198944 | 1.0859834 | 1.0961016 | 1.089469 | 0.8021975 | 0.8265013 |
| Organic anion transporter 3 | 1.1841656 | 1.2085371 | 1.1187407 | 1.0294971 | 0.8862291 | 0.8401815 | 0.778048 |
| N-hydroxy-2-acetylaminofluorene | 1.1828593 | 1.325366 | 0.8816444 | 1.2887497 | 1.0028814 | 1.0628104 | 0.9025569 |
| sulfotransferase (ST1C1) | | | | | | | |
| Integrin beta1 | 1.0944642 | 1.2165554 | 1.2213789 | 1.2302574 | 1.1724662 | 0.788991 | 1.011055 |
| Insulin-like growth factor binding protein 3 | 1.2631353 | 0.9708698 | 1.4693639 | 1.600706 | 1.3652934 | 0.8727799 | 0.9009781 |
| ID-1 | 1.1816947 | 1.0564458 | 0.7407284 | 1.0705792 | 1.256261 | 1.0303708 | 1.0610521 |
| Hepatic lipase | 0.6303916 | 0.5600941 | 0.9810646 | 0.7321705 | 0.8121492 | 0.833428 | 0.8863934 |
| Heme oxygenase | 1.0702342 | 1.2634375 | 1.050937 | 0.8838499 | 1.3869224 | 1.1569924 | 1.0006303 |
| Equilbrative nitrobenzylthioinosine- | 1.0775343 | 1.1715676 | 0.8110661 | 1.0901519 | 0.8949498 | 1.0685555 | 0.8813955 |
| sensitive nucleoside transporter | | | | | | | |
| Epidermal growth factor | 1.3075118 | 1.388108 | 1.3267932 | 1.379375 | 1.1397313 | 1.0111729 | 1.1043909 |
| c-myc | 0.9666544 | 0.9361463 | 0.9351922 | 0.9884995 | 0.9532215 | 1.1028724 | 1.1575606 |
| Carbonic anhydrase III | 1.3327147 | 1.3242418 | 0.4439203 | 1.6906607 | 0.7984101 | 0.5934242 | 1.3014743 |
| Beta-actin | 0.8117186 | 0.6514357 | 1.2112511 | 0.9523154 | 1.039192 | 0.8045793 | 0.983063 |
| Bax (alpha) | 0.9902396 | 0.9984193 | 1.0850557 | 0.9463466 | 0.9335294 | 0.9603785 | 1.1219188 |
| Alpha 1 - inhibitor III | 0.6020309 | 0.8345335 | 0.8800563 | 1.1766325 | 0.8413712 | 0.835433 | 0.9544944 |
| 60S ribosomal protein L6 | 0.9204704 | 0.8722401 | 1.1303384 | 0.9292927 | 1.0307205 | 0.7087612 | 0.7280433 |
| Phase-1 RCT-117 | 1.5715942 | 1.1822119 | 1.3269714 | 1.7546465 | 1.5499606 | 0.8427857 | 0.563715 |
| Phase-1 RCT-102 | 1.1041666 | 0.6304256 | 0.8075329 | 1.1497539 | 0.7008895 | 1.2441186 | 1.2227176 |
| 3-hydroxyisobutyrate dehydrogenase | 0.9585799 | 1.0524415 | 1.1561172 | 1.3428783 | 1.0422641 | 1.0544919 | 0.9660614 |
| ATPase inhibitor (rat mitochondrial IF1 | 1.2636069 | 1.329916 | 1.5637999 | 1.7302519 | 1.2417653 | 0.9412271 | 0.9271659 |
| protein) | | | | | | | |
| Alpha-2-macroglobulin | 0.68129 | 0.925558 | 0.9015269 | 1.1922066 | 0.8921916 | 0.8326822 | 0.8768965 |
| Phase-1 RCT-137 | 1.1025071 | 0.9949787 | 1.280027 | 1.24049 | 0.9097586 | 0.9468865 | 1.0144308 |
| Phase-1 RCT 252 | 0.7153574 | 0.9013612 | 1.1122495 | 0.8003425 | 1.2293086 | 0.847656 | 0.9462754 |
| Phase-1 RCT-65 | 0.9863725 | 0.9358417 | 0.9797127 | 0.9398298 | 1.014891 | 1.0459671 | 0.9322175 |
| Liver fatty acid binding protein | 1.6880068 | 1.0784471 | 2.4441092 | 2.0403361 | 1.4040172 | 0.6607648 | 0.7835433 |
| Carbamyl phosphate synthetase I | 0.7025101 | 0.9568247 | 1.1744307 | 0.7869706 | 1.3029001 | 0.8864681 | 0.9655423 |
| c-jun | 1.1282609 | 1.1265223 | 0.9996491 | 0.9696589 | 1.052074 | 0.9226851 | 0.8870156 |
| Transthyretin | 1.0839955 | 1.2138711 | 1.1192613 | 1.4871801 | 1.2497473 | 0.7778149 | 0.9214144 |
| Aflatoxin B1 aldehyde reductase | 0.9726734 | 0.903594 | 1.0153875 | 1.098501 | 0.557184 | 0.6897222 | 0.9514954 |
| Phase-1 RCT-15 | 1.1782337 | 1.3979107 | 1.3067362 | 1.4754183 | 1.2387602 | 1.048308 | 1.0442855 |
| Insulin-like growth factor I | 1.2942328 | 0.9682131 | 1.4482143 | 1.6749557 | 1.3596144 | 0.8513913 | 0.9096484 |
| Sodium/bile acid cotransporter | 0.7085403 | 0.9230885 | 0.9926173 | 1.2368286 | 0.6697392 | 0.8149502 | 0.839996 |
| Organic cation transporter 3 | 0.9317605 | 0.8896883 | 1.1277324 | 0.9273115 | 1.0678494 | 0.7944236 | 0.9691013 |
| Gadd153 | 1.0192113 | 1.1481435 | 1.1184 | 0.9279088 | 0.9995337 | 1.050868 | 1.1407677 |
| Phase-1 RCT-109 | 0.9942706 | 0.9351791 | 1.2235347 | 1.1790587 | 1.176983 | 0.84171 | 0.8985793 |
| Phase-1 RCT-88 | 0.7264431 | 1.1169629 | 1.0946724 | 1.4547548 | 1.26784 | 1.0965971 | 0.9389705 |
| Ubiquitin conjugating enzyme (RAD 6 | 1.056306 | 0.9160739 | 1.2625785 | 1.149306 | 1.0256733 | 0.906728 | 0.9319413 |
| homologue) | | | | | | | |
| Glucose-regulated protein 78 | 1.2728306 | 1.1436148 | 1.2474352 | 1.093518 | 1.0502551 | 0.6099976 | 0.9194965 |
| Ribosomal protein L13A | 1.1706531 | 1.0532341 | 1.4137654 | 1.421048 | 1.328438 | 0.8104442 | 0.85232 |
| Monocyte chemotactic protein receptor | 1.0422331 | 1.1259696 | 0.8738682 | 0.9581001 | 0.9333133 | 1.1050152 | 1.0381026 |
| (CCR2) | | | | | | | |
| Fas antigen | 1.1215537 | 1.0569416 | 1.1097461 | 1.234744 | 1.3086771 | 0.9073492 | 0.9926708 |
| Calpactin I heavy chain | 1.0605546 | 0.9715934 | 1.120898 | 1.00047 | 0.9845949 | 1.0746492 | 1.0713623 |
| Uncoupling protein 2 | 1.0631307 | 1.0463644 | 1.0204524 | 1.1526188 | 1.1209371 | 1.024316 | 0.9358413 |
| Phase-1 RCT-168 | 0.8216755 | 0.8979535 | 0.840656 | 0.8800999 | 1.1638951 | 1.0348994 | 1.1002206 |
| Phase-1 RCT-154 | 1.0592324 | 1.1842642 | 0.7402327 | 1.0496657 | 0.9854668 | 0.993188 | 1.0617615 |
| Superoxide dismutase Mn | 1.0324523 | 1.2556896 | 1.2993141 | 1.2324454 | 1.3669215 | 0.9540775 | 1.0481809 |
| Phase-1 RCT-214 | 1.003662 | 0.8626469 | 1.1493657 | 0.9719885 | 0.9084491 | 1.1195346 | 1.0576253 |
| Phase-1 RCT-225 | 0.522997 | 0.887614 | 0.8442676 | 1.1305418 | 0.6477811 | 1.572599 | 1.3345952 |
| Phase-1 RCT-181 | 0.8554987 | 0.8911095 | 1.0667897 | 1.0034891 | 1.1055145 | 1.1227278 | 0.9401004 |
| Nucleoside diphosphate kinase beta | 0.8357461 | 0.7585334 | 1.2227043 | 0.9956796 | 0.7809755 | 0.9384845 | 0.9721617 |
| isoform | | | | | | | |
| Extracellular-signal-regulated kinase 1 | 1.3030353 | 1.3173125 | 0.5741962 | 1.4144478 | 0.8290404 | 0.6853876 | 1.1736448 |
| {Ribosomal protein L6} | 0.7790495 | 0.8136499 | 1.2115475 | 0.8855131 | 1.0823371 | 0.8178982 | 0.8188576 |
| Aldehyde dehydrogenase, microsomal | 0.7770435 | 0.786681 | 1.0212852 | 0.991674 | 1.0396155 | 0.908682 | 0.8805981 |
| p53 | 1.0615584 | 0.8702718 | 0.9221427 | 0.9652913 | 0.8431112 | 0.8290238 | 0.8863393 |
| Phase-1 RCT-239 | 0.7754941 | 0.7583675 | 0.831528 | 0.9372528 | 1.0222839 | 1.093111 | 1.1612922 |
| Macrophage inflammatory protein-2 alpha | 1.2062088 | 1.3168913 | 1.1146048 | 1.2084594 | 1.2095413 | 1.1940523 | 1.0553911 |
| IgE binding protein | 1.1622323 | 1.1153607 | 1.0710549 | 1.0563564 | 0.9232249 | 1.140696 | 1.0545754 |
| Phase-1 RCT-205 | 0.9710983 | 0.9269777 | 0.8820342 | 0.8636512 | 0.9108619 | 1.1215739 | 1.1375027 |
| Melanoma-associated antigen ME491 | 1.0776676 | 0.9592353 | 0.9837838 | 0.9488621 | 1.0727094 | 1.1214076 | 1.0240333 |
| Phase-1 RCT-242 | 0.9946978 | 0.9852824 | 0.7197771 | 0.8528365 | 0.8596031 | 1.2176791 | 1.3520192 |
| Enolase alpha | 0.7682589 | 0.652586 | 1.3186885 | 0.7474269 | 0.6501082 | 1.0868618 | 1.1103483 |
| Thymosin beta-10 | 0.9581566 | 0.9614191 | 1.2976882 | 1.1868483 | 1.2924932 | 1.0991974 | 1.1427506 |
| Cofilin | 1.0124148 | 1.1405263 | 1.0752945 | 1.2717332 | 1.0648916 | 1.0198925 | 0.9815837 |
| Carbonyl reductase | 1.0295571 | 1.0184896 | 0.821646 | 0.9495655 | 0.9466109 | 1.132738 | 1.1055502 |
| MAP kinase kinase | 0.9355528 | 0.9550098 | 0.9999999 | 0.9529578 | 1.0449091 | 0.8318034 | 0.8988312 |
| Phase-1 RCT-162 | 0.9915647 | 0.9774251 | 1.2605319 | 1.0328745 | 1.0474048 | 0.9311755 | 0.9006643 |
| Phase-1 RCT-72 | 0.9993311 | 1.0555423 | 0.9232943 | 0.7272395 | 0.7384659 | 1.0268527 | 0.80545 |
| Ribosomal protein S8 | 0.9744363 | 0.964522 | 1.3615699 | 1.0968428 | 1.0054257 | 0.8686676 | 0.7956931 |
| Proliferating cell nuclear antigen gene | 1.0299517 | 1.0155622 | 0.9386392 | 0.8931773 | 1.0104059 | 1.0435642 | 1.150526 |
| Phase-1 RCT-192 | 1.0849802 | 1.0831294 | 1.1885597 | 1.2200744 | 1.1015702 | 0.9140614 | 0.8477501 |
| IkB-a | 0.6232292 | 0.6108021 | 1.0166115 | 0.6802352 | 0.7139206 | 0.8872797 | 0.7946403 |
| Endogenous retroviral sequence, 5′ and | 0.8339272 | 1.0410879 | 0.6782672 | 1.2246965 | 1.0576297 | 1.0766603 | 0.9874615 |
| 3′ LTR | | | | | | | |
| Phase-1 RCT-37 | 0.8664403 | 0.8622214 | 1.1978749 | 0.9125728 | 0.9986844 | 1.0883018 | 0.9278949 |
| Matrix metalloproteinase-1 | 1.1782049 | 1.299454 | 1.4858861 | 1.8867835 | 1.721907 | 0.9681888 | 1.005486 |
| Cyclin G | 1.133725 | 1.2393553 | 1.0897766 | 0.9800901 | 0.9793899 | 1.0954934 | 1.1774526 |
| DNA polymerase beta | 1.0615273 | 0.9834485 | 1.3504279 | 1.1214584 | 1.0202212 | 0.8664167 | 0.9266228 |
| Phase-1 RCT-127 | 1.3506311 | 1.1371017 | 1.0570236 | 1.2682362 | 1.0974578 | 1.1324238 | 1.000432 |
| Multidrug resistant protein-2 | 1.2207487 | 1.201642 | 1.0382733 | 1.05017 | 1.4639835 | 0.7760969 | 0.9476298 |
| Ribosomal protein S9 | 1.0538338 | 0.8965895 | 1.2827181 | 1.1281371 | 1.0102783 | 0.9698018 | 0.9735779 |
| Phase-1 RCT-12 | 0.9134523 | 0.739342 | 1.0464675 | 0.9814751 | 0.8225593 | 0.944835 | 1.0256195 |
| Beta-tubulin, class I | 0.9318978 | 0.7198955 | 1.0591851 | 0.9518901 | 0.7940686 | 0.9258713 | 1.1258496 |
| Argininosuccinate lyase | 1.1616768 | 1.2649676 | 1.1984321 | 1.3802234 | 1.755123 | 0.9588067 | 0.795754 |
| Elongation factor-1 alpha | 0.8699598 | 0.8452169 | 1.1686779 | 1.1082381 | 1.252189 | 0.8431848 | 0.874201 |
| Phase-1 RCT-55 | 0.8757476 | 0.78344 | 1.060502 | 0.6779376 | 0.8216571 | 0.996997 | 0.8684857 |
| Macrophage inflammatory protein-1 alpha | 1.1110477 | 1.1038648 | 0.6742683 | 1.1218735 | 0.7743608 | 1.2778699 | 1.2810473 |
| c-H-ras | 1.0282845 | 1.0176795 | 1.0575941 | 0.8977391 | 1.0856918 | 0.8866343 | 0.9799438 |
| Multidrug resistant protein-1 | 1.1456487 | 1.1847847 | 1.0356126 | 1.1278173 | 1.369567 | 0.8600729 | 0.9240845 |
| T-cell cyclophilin | 1.0419444 | 0.9903078 | 1.379597 | 1.1566294 | 1.0391054 | 0.8412679 | 0.8817307 |
| Phase-1 RCT-185 | 0.8375527 | 0.8364909 | 1.0175976 | 1.0495721 | 0.835218 | 1.020658 | 0.9409059 |
| 25-DX | 1.0412554 | 0.930836 | 1.0203073 | 1.292047 | 1.0641023 | 0.8263314 | 0.9208442 |
| Ref-1 | 0.9130434 | 1.0976537 | 1.1708761 | 0.8354771 | 0.8001242 | 0.967844 | 1.1454488 |
| Apolipoprotein CIII | 0.7803204 | 0.8092343 | 0.8069858 | 0.9994127 | 1.0208783 | 1.0288476 | 1.1433631 |
| NADPH quinone oxidoreductase-1 (DT- | 0.8930947 | 1.0374857 | 1.1497707 | 0.9539757 | 0.7637941 | 1.073212 | 0.9830514 |
| diaphorase) | | | | | | | |
| Pyruvate kinase, muscle | 0.8897784 | 0.8429113 | 1.0722612 | 0.7995566 | 0.8095454 | 0.9671666 | 0.9089285 |
| | HYD | HYD | HYD | ISON | ISON | ISON | ISON |
| Compound-Dose (2) | 250 | 1000 | 1000 | 50 | 50 | 50 | 200 |
| Animal Number (3) | 1226 | 1234 | 1235 | 1944 | 1945 | 1946 | 195 |
| Liver Toxicity Necrosis Classification (4) | no | no | no | no | no | no | no |
| Gene Name (5) | | | | | | | |
| Phase-1 RCT-78 | 1.034266 | 1.1473181 | 0.9408748 | 1.0194309 | 0.9971745 | 1.0182787 | 1.02161 |
| Gamma-actin, cytoplasmic | 0.795939 | 1.048167 | 0.8047211 | 1.0642765 | 0.8944653 | 0.9833431 | 0.754591 |
| Matrin F/G | 0.920652 | 0.9778423 | 0.9066948 | 1.0328751 | 1.2618638 | 1.1104137 | 1.035175 |
| Zinc finger protein | 1.106215 | 1.0233219 | 1.4582646 | 0.9420566 | 1.0802664 | 0.9580917 | 1.049549 |
| Cathepsin L, sequence 2 | 0.8018299 | 0.9532248 | 1.0104743 | 1 | 1.0224717 | 0.8911307 | 0.903525 |
| Gadd45 | 0.728824 | 0.9634249 | 0.9912524 | 1.0940017 | 0.9574993 | 1.1608081 | 1.186812 |
| Phase-1 RCT-144 | 1.015557 | 1.0043815 | 1.0602081 | 1.0162618 | 0.9683387 | 0.9247011 | 1.045972 |
| Phase-1 RCT-145 | 0.9700469 | 1.139133 | 1.2130365 | 0.9665396 | 0.8912392 | 0.9005471 | 1.016061 |
| Phase-1 RCT-50 | 1.0242348 | 1.1001874 | 0.8884317 | 1.0624366 | 0.9326247 | 1.0237162 | 1.224857 |
| Phase-1 RCT-92 | 0.911805 | 1.0378847 | 0.9751588 | 0.8411602 | 0.9610061 | 0.9469422 | 0.9242089 |
| Phase-1 RCT-89 | 0.9604561 | 1.1150047 | 0.9410381 | 0.9290174 | 1.0099242 | 1.1045071 | 1.014066 |
| 14-3-3 zeta | 0.9759859 | 0.9818059 | 0.8350681 | 1.0155889 | 1.06171 | 1.0732108 | 0.977501 |
| Dynamin-1 (D100) | 0.9384303 | 0.9794911 | 0.9806246 | 0.9577717 | 0.9284474 | 1.0373116 | 0.942505 |
| Insulin-like growth factor binding protein 1 | 1.084 | 1.0188805 | 0.871156 | 0.9486679 | 1.0242032 | 0.9198135 | 0.733571 |
| L-gulono-gamma-lactone oxidase | 0.7439837 | 0.9862371 | 0.7300397 | 1.0235599 | 0.9698631 | 1.226455 | 1.226751 |
| Ornithine decarboxylase | 0.9651812 | 1.1404023 | 1.0987549 | 1.2647718 | 1.1232225 | 1.0789171 | 1.014437 |
| PAR interacting protein | 0.9550558 | 0.9731146 | 1.0093625 | 0.9815915 | 0.9386678 | 0.964671 | 0.98730 |
| Phase-1 RCT-128 | 0.8150839 | 0.9913922 | 0.8352602 | 0.8431473 | 0.8913848 | 1.0398548 | 0.828966 |
| Phase-1 RCT-180 | 1.2033175 | 0.9684245 | 1.1311677 | 0.9897975 | 1.0565788 | 1.1423079 | 0.793752 |
| Phase-1 RCT-182 | 0.8256747 | 0.9218923 | 0.8350927 | 0.812234 | 0.9394742 | 0.9959853 | 0.950599 |
| Phase-1 RCT-207 | 1.0193889 | 1.0580065 | 1.1606895 | 1.0009496 | 1.0920736 | 1.0211989 | 1.035437 |
| Phase-1 RCT-213 | 1.1505667 | 1.1204804 | 1.0132308 | 0.9636098 | 1.0108196 | 0.9673724 | 0.917801 |
| Phase-1 RCT-256 | 0.8691208 | 0.9481896 | 0.9444213 | 1.0300735 | 1.1105057 | 1.1960034 | 1.0152981 |
| Phase-1 RCT-258 | 1.0420799 | 1.4051067 | 1.0202287 | 1.0200604 | 1.0380136 | 1.0142348 | 1.02033 |
| Phase-1 RCT-264 | 1.0263332 | 1.0582463 | 0.6371872 | 0.7968336 | 0.9198785 | 0.8815069 | 0.954901 |
| Phase-1 RCT-271 | 1.0852008 | 0.9951937 | 1.2707809 | 0.9527961 | 1.0058054 | 0.8527886 | 0.921833 |
| Phase-1 RCT-288 | 0.9895923 | 1.0537738 | 0.8574683 | 0.7401016 | 0.9785434 | 1.0032429 | 0.858913 |
| Phase-1 RCT-38 | 0.9111179 | 0.9649018 | 1.048939 | 1.0951434 | 1.2021264 | 1.2582306 | 0.986959 |
| Phase-1 RCT-39 | 0.9626771 | 0.9242961 | 1.2017114 | 0.983249 | 1.064578 | 1.0431495 | 1.202496 |
| Phase-1 RCT-68 | 1.0211445 | 1.1762831 | 0.9866021 | 0.9402037 | 0.9509398 | 0.955255 | 0.9865559 |
| Phase-1 RCT-33 | 1.2184277 | 0.8701803 | 0.8673543 | 0.7797558 | 0.8877618 | 0.9973171 | 1.0160275 |
| Phase-1 RCT-36 | 1.0955002 | 0.9205775 | 0.9878899 | 0.8712091 | 0.9499825 | 0.9418746 | 1.0428163 |
| 17-beta hydroxysteroid dehydrogenase, | 0.6588196 | 0.973591 | 1.1435977 | 1.1573794 | 1.4463253 | 0.9232926 | 0.8690406 |
| type 2 | | | | | | | |
| Senescence marker protein-30 | 0.777746 | 0.9937916 | 0.7182488 | 1.3233312 | 1.0193983 | 1.2239228 | 0.9022475 |
| Ribosomal protein S17 | 0.9578706 | 1.0762674 | 1.2687153 | 0.8734309 | 0.8419237 | 0.9253905 | 0.7330986 |
| Phase-1 RCT-83 | 1.1187413 | 1.1395622 | 1.2520956 | 0.9270518 | 0.8911226 | 0.8085052 | 1.0760432 |
| Phase-1 RCT-49 | 1.0513809 | 0.9483959 | 1.0876163 | 0.9830225 | 0.8888272 | 0.9161264 | 1.0405135 |
| Phase-1 RCT-48 | 0.9259428 | 1.1625558 | 0.9046681 | 1.2125466 | 1.1998762 | 1.2521398 | 1.3124695 |
| Phase-1 RCT-40 | 0.9656231 | 1.1433935 | 0.9270871 | 0.8836049 | 0.8517246 | 0.9169716 | 0.7942146 |
| Phase-1 RCT-296 | 0.9901228 | 0.8196391 | 0.6180413 | 1.027992 | 0.9221876 | 1.3798724 | 1.0788025 |
| Phase-1 RCT-295 | 0.9769669 | 1.0745223 | 0.9050343 | 0.9973648 | 0.9680795 | 1.0265961 | 0.9429637 |
| Phase-1 RCT-291 | 0.874132 | 0.9027695 | 0.9773631 | 0.9508338 | 1.0276227 | 1.1437378 | 0.9298418 |
| Phase-1 RCT-270 | 0.8594698 | 1.1029173 | 0.8799874 | 0.9743679 | 0.8952242 | 1.1103922 | 0.89437 |
| Phase-1 RCT-241 | 1.0909806 | 1.1078087 | 1.1237997 | 1.0777122 | 1.0831321 | 1.1070241 | 0.9853983 |
| Phase-1 RCT-191 | 1.1571751 | 1.0781744 | 1.0791872 | 1.0272368 | 0.9955303 | 0.9930984 | 1.0519683 |
| Phase-1 RCT-189 | 0.9996302 | 1.1124154 | 1.0427809 | 1.0262027 | 1.0594009 | 1.091627 | 1.0108492 |
| Phase-1 RCT-179 | 0.9934368 | 1.0480126 | 0.8813787 | 0.8530107 | 1.0205733 | 0.9997062 | 0.9310848 |
| Phase-1 RCT-152 | 0.906831 | 1.0134318 | 1.0687509 | 0.8248254 | 0.8833249 | 0.926147 | 0.8266792 |
| Phase-1 RCT-123 | 1.1419148 | 1.2894577 | 1.104733 | 1.0001745 | 0.9682857 | 0.9543611 | 1.108561 |
| Paraoxonase 1 | 0.7698115 | 0.8397236 | 0.7640968 | 0.8197056 | 0.8579436 | 0.9522977 | 0.7212046 |
| Organic anion transporter 3 | 0.6517652 | 1.0557644 | 0.8243237 | 1.1395832 | 1.1664703 | 0.9552745 | 1.3530952 |
| N-hydroxy-2-acetylaminofluorene | 0.9921995 | 1.1037802 | 1.0191003 | 0.8369829 | 0.8877028 | 0.6886919 | 0.8734899 |
| sulfotransferase (ST1C1) | | | | | | | |
| Integrin beta1 | 1.0007664 | 0.9368945 | 0.8638666 | 1.138228 | 1.0484308 | 1.0606141 | 1.0103307 |
| Insulin-like growth factor binding protein 3 | 1.0551021 | 0.9522151 | 0.7742635 | 1.0059398 | 0.9932745 | 1.197857 | 1.0487727 |
| ID-1 | 1.1400918 | 1.009254 | 0.9498615 | 1.1598524 | 1.1448548 | 0.9106543 | 0.805737 |
| Hepatic lipase | 0.9409493 | 0.8627411 | 0.6987075 | 0.8321517 | 0.9855757 | 1.0348941 | 0.6967887 |
| Heme oxygenase | 0.9626806 | 1.2185916 | 1.0401386 | 0.9930304 | 0.9999794 | 1.0400425 | 1.0586224 |
| Equilbrative nitrobenzylthioinosine- | 0.9544603 | 1.0804304 | 1.1566039 | 0.9096631 | 0.905031 | 0.8184449 | 0.9190199 |
| sensitive nucleoside transporter | | | | | | | |
| Epidermal growth factor | 0.9754682 | 0.9556877 | 1.0565844 | 1.0365705 | 0.9829786 | 1.0521398 | 1.0301532 |
| c-myc | 0.9869386 | 0.852393 | 1.0101552 | 1.1288445 | 0.9305725 | 1.0381058 | 1.1311163 |
| Carbonic anhydrase III | 0.5285785 | 1.4373379 | 0.7654026 | 0.8170907 | 0.9959975 | 0.7321452 | 0.7210667 |
| Beta-actin | 0.8374251 | 0.9729326 | 0.9561148 | 0.8947739 | 1.0470562 | 1.0591486 | 0.6745468 |
| Bax (alpha) | 1.1170973 | 1.0068831 | 0.7727151 | 1.3098528 | 1.181285 | 1.2065555 | 1.0946876 |
| Alpha 1 - inhibitor III | 0.8765229 | 0.8041059 | 0.6078807 | 0.7007685 | 1.0766128 | 0.9356605 | 0.5285459 |
| 60S ribosomal protein L6 | 0.9785352 | 0.8776573 | 1.1309985 | 0.9794306 | 0.9129872 | 0.9420473 | 0.8210973 |
| Phase-1 RCT-117 | 0.9726462 | 1.031061 | 1.0301884 | 1.1392814 | 1.3749171 | 1.1821969 | 1.4298959 |
| Phase-1 RCT-102 | 0.8841807 | 0.9952309 | 0.8145928 | 1.4256184 | 1.1631548 | 1.2033422 | 1.4304422 |
| 3-hydroxyisobutyrate dehydrogenase | 0.8081374 | 1.1353905 | 0.9900101 | 0.9370501 | 1.0357172 | 1.0040869 | 1.0819181 |
| ATPase inhibitor (rat mitochondrial IF1 | 0.9895025 | 0.9825305 | 1.1011666 | 0.7399393 | 0.7790721 | 0.8056236 | 0.8708082 |
| protein) | | | | | | | |
| Alpha-2-macroglobulin | 0.8696209 | 0.8298237 | 0.6123568 | 0.7536168 | 1.1396079 | 1.0046276 | 0.6584203 |
| Phase-1 RCT-137 | 0.9430574 | 1.0317522 | 1.048511 | 0.855108 | 0.9225142 | 0.9980855 | 0.8552747 |
| Phase-1 RCT 252 | 0.7264024 | 0.9709659 | 0.8858083 | 0.7477737 | 0.7929267 | 1.0839841 | 0.7845403 |
| Phase-1 RCT-65 | 1.1789769 | 1.1039528 | 1.0191324 | 1.2497288 | 1.1730624 | 1.1810716 | 1.140557 |
| Liver fatty acid binding protein | 0.9840676 | 0.9583159 | 0.7265947 | 0.9604703 | 1.0102272 | 1.0955236 | 1.0389311 |
| Carbamyl phosphate synthetase I | 0.7326866 | 0.9838259 | 0.919436 | 0.7875639 | 0.8311334 | 1.1897417 | 0.7513248 |
| c-jun | 0.8810245 | 0.847995 | 0.8381087 | 1.0519667 | 1.0308313 | 1.5700697 | 1.3536562 |
| Transthyretin | 0.7760623 | 0.8805981 | 0.6326885 | 0.7867537 | 0.8372355 | 0.7782328 | 0.7506251 |
| Aflatoxin B1 aldehyde reductase | 0.672212 | 1.0571655 | 0.9331558 | 0.9028075 | 0.8054457 | 0.8668265 | 0.749303 |
| Phase-1 RCT-15 | 1.1508646 | 1.1407027 | 1.0726427 | 1.1680273 | 1.2177463 | 1.0990577 | 1.0497311 |
| Insulin-like growth factor I | 0.9703988 | 0.8730183 | 0.6105285 | 1.0402808 | 1.0515665 | 1.372127 | 0.9400312 |
| Sodium/bile acid cotransporter | 0.8765463 | 0.9859971 | 0.903415 | 0.9028674 | 0.8191668 | 0.6603963 | 0.9097055 |
| Organic cation transporter 3 | 1.0576495 | 1.0062274 | 1.119748 | 1.0505072 | 0.9475793 | 0.9621199 | 0.869647 |
| Gadd153 | 1.0127143 | 0.9793034 | 0.8198498 | 1.1825036 | 1.1234419 | 1.2247586 | 1.06311 |
| Phase-1 RCT-109 | 1.0083104 | 0.8918374 | 0.8591611 | 0.9314303 | 0.9269586 | 1.0021944 | 0.9627958 |
| Phase-1 RCT-88 | 0.808735 | 1.0067487 | 0.9533916 | 0.9709692 | 1.0028412 | 0.9076648 | 1.0592301 |
| Ubiquitin conjugating enzyme (RAD 6 | 0.9816362 | 1.0939724 | 1.1254756 | 0.9222832 | 0.9855831 | 0.8320127 | 0.9368997 |
| homologue) | | | | | | | |
| Glucose-regulated protein 78 | 0.9069001 | 0.8693146 | 1.0267153 | 1.1821052 | 1.2917292 | 1.3966837 | 0.9924203 |
| Ribosomal protein L13A | 1.02045 | 0.9093405 | 0.9349457 | 0.9874277 | 1.0427496 | 1.122157 | 0.9749976 |
| Monocyte chemotactic protein receptor | 1.1600652 | 1.0875976 | 1.1388941 | 1.1263497 | 1.177159 | 1.0684716 | 1.0511105 |
| (CCR2) | | | | | | | |
| Fas antigen | 1.0573004 | 0.9387041 | 1.0408777 | 1.0164456 | 1.107393 | 1.120058 | 0.9445268 |
| Calpactin I heavy chain | 1.0916545 | 1.0442952 | 1.0826102 | 1.0868433 | 0.991708 | 1.0956435 | 1.0816089 |
| Uncoupling protein 2 | 1.0590008 | 0.9818838 | 1.1686265 | 1.1314402 | 1.0040936 | 0.9565954 | 0.891229 |
| Phase-1 RCT-168 | 1.1047134 | 1.0354748 | 1.0430973 | 1.0322655 | 1.0254029 | 1.0594311 | 0.9736002 |
| Phase-1 RCT-154 | 0.9735935 | 1.1061276 | 1.2570789 | 0.9419498 | 0.8716424 | 0.9093918 | 1.0408293 |
| Superoxide dismutase Mn | 1.0625435 | 1.097389 | 1.039545 | 1.0340834 | 0.9925498 | 1.0761425 | 1.1318398 |
| Phase-1 RCT-214 | 1.1579489 | 1.0738946 | 0.941444 | 0.9238302 | 1.0860199 | 1.076079 | 0.91649 |
| Phase-1 RCT-225 | 1.285982 | 1.036237 | 0.9387441 | 1.695666 | 1.6707928 | 1.3424957 | 1.6355484 |
| Phase-1 RCT-181 | 1.0271485 | 0.9730768 | 0.9978963 | 1.1069435 | 1.0369626 | 1.1350183 | 1.021185 |
| Nucleoside diphosphate kinase beta | 0.9941291 | 0.9584973 | 1.2097692 | 0.9245014 | 1.0037459 | 0.9943929 | 0.8971064 |
| isoform | | | | | | | |
| Extracellular-signal-regulated kinase 1 | 0.8411101 | 1.0854298 | 0.927424 | 0.9733957 | 1.0919156 | 0.8168558 | 0.9482259 |
| {Ribosomal protein L6} | 0.8512244 | 1.0134186 | 0.9761244 | 0.9173635 | 0.9009833 | 1.0412807 | 0.924678 |
| Aldehyde dehydrogenase, microsomal | 1.0180565 | 0.9737065 | 0.8945173 | 0.9007163 | 0.9604214 | 0.975256 | 0.894158 |
| p53 | 1.086822 | 0.9691505 | 1.0261878 | 1.1170901 | 1.0284007 | 1.0373182 | 0.9431335 |
| Phase-1 RCT-239 | 1.2366444 | 0.8729991 | 0.9573811 | 1.0151432 | 1.1614729 | 0.9522174 | 0.980117 |
| Macrophage inflammatory protein-2 alpha | 0.9621005 | 0.9755085 | 0.933286 | 1.1271849 | 1.1432244 | 1.0183791 | 1.1889317 |
| IgE binding protein | 1.1020317 | 1.1198962 | 1.2479137 | 1.0166264 | 0.9840332 | 0.8891301 | 1.0031376 |
| Phase-1 RCT-205 | 1.0816698 | 0.9363962 | 1.1389754 | 0.9742101 | 0.9483627 | 0.946687 | 1.049421 |
| Melanoma-associated antigen ME491 | 1.1955447 | 0.9312111 | 1.12439 | 0.8415554 | 0.8630532 | 0.7345863 | 0.8408212 |
| Phase-1 RCT-242 | 1.0448416 | 0.9601321 | 1.2883465 | 1.0367895 | 0.8837277 | 0.9226682 | 1.1409768 |
| Enolase alpha | 0.9155261 | 0.8598598 | 1.2823938 | 0.9423048 | 0.9809009 | 1.0749587 | 0.530104 |
| Thymosin beta-10 | 0.9084089 | 0.9365493 | 1.0656102 | 1.0439136 | 1.0469486 | 1.0450234 | 0.802376 |
| Cofilin | 1.0454087 | 1.1049377 | 1.005104 | 0.9384833 | 0.9831431 | 0.8899224 | 0.9781682 |
| Carbonyl reductase | 1.1249284 | 1.1036372 | 0.8331498 | 1.1370302 | 1.0101993 | 1.0561584 | 0.954021 |
| MAP kinase kinase | 0.8678213 | 0.8934802 | 0.7871419 | 1.0302216 | 0.9740614 | 1.1138277 | 0.8661429 |
| Phase-1 RCT-162 | 0.996208 | 1.0644124 | 0.994513 | 0.9608235 | 0.9470944 | 0.9668406 | 1.1346902 |
| Phase-1 RCT-72 | 1.0779855 | 0.9876357 | 1.4557978 | 1.0566113 | 0.9742001 | 1.0043231 | 1.2021968 |
| Ribosomal protein S8 | 0.9204715 | 0.967875 | 1.115866 | 0.883599 | 0.8219397 | 0.9268531 | 0.798643 |
| Proliferating cell nuclear antigen gene | 1.0209485 | 0.9762213 | 0.9370389 | 1.1281192 | 1.055804 | 1.0560468 | 1.0952584 |
| Phase-1 RCT-192 | 0.9836834 | 1.0495647 | 1.1465833 | 0.9126629 | 0.9030663 | 0.9179789 | 0.9721194 |
| IkB-a | 1.0138888 | 0.8912371 | 1.0719646 | 0.9612538 | 1.211948 | 1.0542142 | 0.7009875 |
| Endogenous retroviral sequence, 5′ and | 0.8465937 | 0.9932109 | 0.8529807 | 0.9866827 | 1.1340623 | 1.0196021 | 0.8513667 |
| 3′ LTR | | | | | | | |
| Phase-1 RCT-37 | 1.0052736 | 1 | 1.0819633 | 1.0326657 | 0.9696869 | 1.0565766 | 1.045542 |
| Matrix metalloproteinase-1 | 0.8973067 | 0.9219039 | 0.8589662 | 1.0742313 | 1.0791916 | 0.9637519 | 0.9809301 |
| Cyclin G | 1.2735089 | 1.0770136 | 1.1380818 | 1.1913975 | 1.0550238 | 1.184819 | 1.1171337 |
| DNA polymerase beta | 1.0343568 | 1.0533001 | 1.0863816 | 0.9844819 | 0.9147182 | 0.8857284 | 0.9215249 |
| Phase-1 RCT-127 | 1.0506088 | 1.083276 | 1.1809843 | 1.033628 | 1.0258442 | 0.9318804 | 0.916714 |
| Multidrug resistant protein-2 | 0.8190407 | 1.0389432 | 0.8018182 | 1.0558667 | 1.1992542 | 0.8480302 | 1.0019383 |
| Ribosomal protein S9 | 1.014014 | 1.0585569 | 1.148671 | 0.8722426 | 0.9435788 | 0.8179385 | 0.8541823 |
| Phase-1 RCT-12 | 0.9980109 | 0.9859499 | 0.7494673 | 1.0615547 | 1.091711 | 1.209812 | 1.1394444 |
| Beta-tubulin, class I | 1.1900632 | 1.134998 | 0.9181141 | 1.2401971 | 1.0068047 | 1.3341961 | 1.2115624 |
| Argininosuccinate lyase | 0.9648988 | 1.0250908 | 0.8651165 | 1.0578202 | 0.9828988 | 1.2870694 | 0.9484 |
| Elongation factor-1 alpha | 0.8702958 | 0.855924 | 0.8652465 | 0.9185001 | 1.0568525 | 1.0173639 | 0.6713777 |
| Phase-1 RCT-55 | 1.124772 | 1.004519 | 1.4760666 | 1.1702518 | 1.0224292 | 1.0165466 | 1.1220354 |
| Macrophage inflammatory protein-1 alpha | 1.1778207 | 1.1237075 | 1.3269705 | 1.1721885 | 0.9431278 | 0.9838689 | 1.05019 |
| c-H-ras | 0.952419 | 1.0354452 | 0.9077078 | 0.9974935 | 1.0415013 | 1.0011933 | 1.029662 |
| Multidrug resistant protein-1 | 0.9724335 | 1.0016265 | 0.8667703 | 1.1637654 | 1.2468741 | 0.999822 | 0.947322 |
| T-cell cyclophilin | 0.9509518 | 1.0263171 | 1.0968541 | 0.9493278 | 0.8645422 | 0.786162 | 0.8539541 |
| Phase-1 RCT-185 | 0.8638534 | 0.9288205 | 0.9188306 | 0.8568544 | 0.9085148 | 0.8143076 | 0.9313814 |
| 25-DX | 0.8534478 | 0.930755 | 0.8529316 | 0.9106961 | 1.0469857 | 0.8192875 | 0.8052754 |
| Ref-1 | 0.9817905 | 1.2355361 | 1.198424 | 1.1660105 | 1.1663514 | 1.186697 | 1.1991013 |
| Apolipoprotein CIII | 1.0366315 | 1.0110352 | 1.001873 | 0.8245831 | 1.0104269 | 0.9439934 | 0.8153954 |
| NADPH quinone oxidoreductase-1 (DT- | 0.9441673 | 1.2365863 | 1.3299046 | 1.0784159 | 1.1470591 | 1.456122 | 1.4504772 |
| diaphorase) | | | | | | | |
| Pyruvate kinase, muscle | 1.1075358 | 0.960882 | 1.0314786 | 1.0630391 | 1.1530824 | 1.0142734 | 1.016861 |
| | ISON | ISON | KETO | KETO | KETO | KETO | KETO |
| Compound-Dose (2) | 200 | 200 | 20 | 20 | 20 | 80 | 80 |
| Animal Number (3) | 1955 | 1956 | 2224 | 2225 | 2226 | 2234 | 2235 |
| Liver Toxicity Necrosis Classification (4) | no | no | no | no | no | no | no |
| Gene Name (5) | | | | | | | |
| Phase-1 RCT-78 | 1.2505031 | 1.0377523 | 1.0118381 | 0.9616363 | 1.035498 | 1.0254543 | 0.9333201 |
| Gamma-actin, cytoplasmic | 0.9091364 | 0.8166835 | 1.1659275 | 1.0089909 | 1.0666492 | 1.3458477 | 1.184166 |
| Matrin F/G | 1.1400276 | 1.2635303 | 1.4045409 | 1.1655637 | 1.4520997 | 1.3897663 | 1.318737 |
| Zinc finger protein | 0.9105815 | 1.0777481 | 0.9372621 | 0.9321073 | 0.9492872 | 0.9503603 | 0.9633657 |
| Cathepsin L, sequence 2 | 0.8311736 | 0.9960375 | 1.0514872 | 0.9972921 | 1.1091624 | 1.0340606 | 1.7347724 |
| Gadd45 | 1.0275252 | 0.822902 | 0.8170908 | 0.9309314 | 0.9052916 | 1.2390476 | 0.8384048 |
| Phase-1 RCT-144 | 0.9219737 | 0.9032011 | 1.058249 | 1.0394533 | 1.0436661 | 1.0230348 | 1.0055276 |
| Phase-1 RCT-145 | 0.9350288 | 0.9861513 | 0.9818517 | 1.011452 | 0.9859617 | 0.9848278 | 0.9676733 |
| Phase-1 RCT-50 | 1.1936268 | 1.1080062 | 0.9425652 | 0.9900424 | 1.0237949 | 1.0255345 | 0.83158 |
| Phase-1 RCT-92 | 0.9716088 | 0.9437345 | 0.9174059 | 0.9532075 | 0.75827 | 0.8256272 | 0.8377456 |
| Phase-1 RCT-89 | 1.1050774 | 1.1418126 | 0.9730109 | 1.0146686 | 0.9636989 | 0.9027262 | 0.9640189 |
| 14-3-3 zeta | 0.9843224 | 1.0846046 | 1.0217621 | 0.9547828 | 1.0897404 | 1.0553625 | 1.0029658 |
| Dynamin-1 (D100) | 0.9620059 | 1.0598326 | 0.9682949 | 1.0967411 | 1.0111337 | 1.0323993 | 1.0199403 |
| Insulin-like growth factor binding protein 1 | 0.8184103 | 0.7408934 | 1.0325334 | 1.0386331 | 1.1922686 | 1.2286586 | 1.3332133 |
| L-gulono-gamma-lactone oxidase | 0.9467056 | 1.3205411 | 1.0461835 | 0.9887675 | 0.6515527 | 0.7392632 | 0.6261718 |
| Ornithine decarboxylase | 1.1198183 | 1.3415717 | 0.982779 | 0.9736652 | 1.095561 | 1.060935 | 1.0942605 |
| PAR interacting protein | 0.9482912 | 0.9152525 | 0.9957943 | 0.9863088 | 1.0180666 | 1.0077685 | 0.9724114 |
| Phase-1 RCT-128 | 0.8116711 | 0.9805222 | 0.9075766 | 1.0571285 | 0.9793593 | 1.0480365 | 1.0452617 |
| Phase-1 RCT-180 | 1.0471691 | 0.8750969 | 1.1214033 | 1.0957489 | 0.9149304 | 0.8767978 | 0.9569448 |
| Phase-1 RCT-182 | 0.7679492 | 0.7872985 | 0.985071 | 0.9217381 | 1.0233893 | 0.9017974 | 0.9419976 |
| Phase-1 RCT-207 | 0.9046283 | 1.1958573 | 1.0381937 | 1.1385409 | 1.0940908 | 1.0536605 | 1.1863681 |
| Phase-1 RCT-213 | 0.9062931 | 0.8852346 | 1.0903741 | 1.0118436 | 1.0594568 | 1.0888842 | 1.1103754 |
| Phase-1 RCT-256 | 1.2191864 | 1.2161734 | 1.0931038 | 1.1704457 | 0.9642637 | 0.9558998 | 0.9261541 |
| Phase-1 RCT-258 | 0.9915605 | 1.0237927 | 0.9904916 | 0.9729152 | 1.0457307 | 0.9638249 | 1.0480026 |
| Phase-1 RCT-264 | 0.9839475 | 0.9441998 | 1.0348049 | 0.8927642 | 0.9931333 | 1.4025486 | 0.6813159 |
| Phase-1 RCT-271 | 0.853452 | 0.9415461 | 0.9401302 | 1.0370944 | 1.0319889 | 1.0311526 | 1.1732624 |
| Phase-1 RCT-288 | 0.813502 | 0.8286943 | 1.175627 | 1.0710568 | 1.114426 | 1.1401707 | 1.099057 |
| Phase-1 RCT-38 | 1.1878568 | 1.2816015 | 1.1007507 | 1.1669194 | 0.9802678 | 1.0179424 | 1.0145471 |
| Phase-1 RCT-39 | 1.1031132 | 0.9793644 | 0.9438506 | 1.003461 | 1.075561 | 1.0368315 | 0.9455866 |
| Phase-1 RCT-68 | 0.9795846 | 1.1217321 | 0.9523327 | 0.9485471 | 0.9610996 | 1.0099264 | 0.8937842 |
| Phase-1 RCT-33 | 0.985803 | 0.610948 | 0.9632369 | 1.0531303 | 0.8633063 | 0.8209232 | 1.088969 |
| Phase-1 RCT-36 | 1.0526359 | 0.8600779 | 0.9551979 | 1.0035325 | 0.9150101 | 0.8687378 | 1.0515262 |
| 17-beta hydroxysteroid dehydrogenase, | 1.0986844 | 1.0047932 | 1.0551414 | 0.8188983 | 1.877573 | 0.6396263 | 0.7812937 |
| type 2 | | | | | | | |
| Senescence marker protein-30 | 0.896805 | 1.1247984 | 0.8957754 | 0.7546618 | 0.9513542 | 0.928715 | 0.777787 |
| Ribosomal protein S17 | 0.749037 | 0.835564 | 1.0044625 | 1.1093985 | 1.030796 | 1.1335502 | 1.6356288 |
| Phase-1 RCT-83 | 0.752061 | 0.7939065 | 0.871072 | 1.1157857 | 0.8882832 | 1.0542574 | 0.8965337 |
| Phase-1 RCT-49 | 1.0135181 | 0.9343741 | 0.9953609 | 1.0347002 | 1.0472064 | 1.0431451 | 1.061106 |
| Phase-1 RCT-48 | 1.3517832 | 1.317018 | 1.1085684 | 1.1481524 | 1.0495578 | 1.0813711 | 0.7876428 |
| Phase-1 RCT-40 | 0.7762284 | 0.9037656 | 0.9339806 | 0.8956168 | 1.1672821 | 1.1384894 | 0.9169989 |
| Phase-1 RCT-296 | 0.7201648 | 0.962347 | 0.9902157 | 0.7501596 | 0.9114901 | 0.7135879 | 1.124458 |
| Phase-1 RCT-295 | 0.9631099 | 1.0642984 | 1.1340121 | 1.0690392 | 1.1180123 | 1.1601406 | 1.1836747 |
| Phase-1 RCT-291 | 1.0724095 | 1.0158128 | 1.065829 | 1.038503 | 1.1050476 | 0.9912725 | 0.9279608 |
| Phase-1 RCT-270 | 0.9365773 | 1.0888602 | 1.0313213 | 0.9082527 | 1.1331205 | 1.288894 | 0.7135239 |
| Phase-1 RCT-241 | 0.9740052 | 0.9459438 | 0.9572519 | 0.8839588 | 0.9089949 | 0.876012 | 0.8727469 |
| Phase-1 RCT-191 | 1.1723387 | 1.1965928 | 1.1272738 | 1.1577923 | 1.1821475 | 1.186199 | 1.1166553 |
| Phase-1 RCT-189 | 1.1283184 | 1.0231994 | 1.0282491 | 1.1416671 | 1.1295162 | 1.3547397 | 0.9674904 |
| Phase-1 RCT-179 | 0.7571484 | 0.8324707 | 0.9471558 | 1.0014976 | 1.0298103 | 1.0329587 | 0.9929803 |
| Phase-1 RCT-152 | 0.7467286 | 0.7784706 | 0.9982155 | 1.1383021 | 1.0854201 | 1.0939046 | 1.4144182 |
| Phase-1 RCT-123 | 1.1538095 | 1.0657519 | 0.9964008 | 1.0449672 | 0.8812012 | 0.9529057 | 0.9061538 |
| Paraoxonase 1 | 0.6394304 | 0.6252485 | 0.9356232 | 0.8264229 | 0.8612702 | 0.9206684 | 0.9118621 |
| Organic anion transporter 3 | 1.2022672 | 1.2891245 | 0.9076735 | 1.0143822 | 1.1660697 | 0.9028798 | 1.3421004 |
| N-hydroxy-2-acetylaminofluorene | 0.7181792 | 0.8782097 | 0.971423 | 1.0283145 | 1.0129206 | 0.853038 | 0.6969199 |
| sulfotransferase (ST1C1) | | | | | | | |
| Integrin beta1 | 1.3182944 | 1.0344393 | 1.1648862 | 1.1730943 | 1.3319616 | 1.2326119 | 1.2749877 |
| Insulin-like growth factor binding protein 3 | 1.1122724 | 0.937466 | 1.0152212 | 1.0725693 | 0.9895595 | 1.1608726 | 0.9602186 |
| ID-1 | 0.7540472 | 0.9756971 | 0.9046503 | 0.9635615 | 0.9886121 | 0.8877909 | 1.3820623 |
| Hepatic lipase | 0.5865308 | 0.6182911 | 0.9055429 | 0.971004 | 0.8492038 | 0.9953846 | 0.7546095 |
| Heme oxygenase | 0.8929768 | 0.654018 | 0.8055482 | 0.8893434 | 0.798218 | 1.1525307 | 0.9958195 |
| Equilbrative nitrobenzylthioinosine- | 0.8099765 | 0.8839169 | 1.0333087 | 1.048104 | 1.0332998 | 0.8911779 | 0.7344196 |
| sensitive nucleoside transporter | | | | | | | |
| Epidermal growth factor | 1.0434536 | 0.9994557 | 0.9539195 | 0.931231 | 0.9462681 | 0.9375359 | 0.9289916 |
| c-myc | 1.0549672 | 0.9236338 | 1.0265619 | 1.0435165 | 0.9481313 | 1.1485606 | 0.9867781 |
| Carbonic anhydrase III | 0.5428243 | 0.8057871 | 0.9288065 | 0.7199903 | 0.9923616 | 0.630214 | 0.3300868 |
| Beta-actin | 0.7455059 | 0.727115 | 1.261218 | 1.1297102 | 1.3523881 | 1.4099844 | 1.2310386 |
| Bax (alpha) | 1.2154232 | 1.1708492 | 0.9168659 | 0.9538658 | 1.0041071 | 1.0121685 | 1.0483627 |
| Alpha 1 - inhibitor III | 0.5299165 | 0.773895 | 1.0791608 | 0.8547216 | 1.4150712 | 1.1821705 | 1.0094987 |
| 60S ribosomal protein L6 | 0.854069 | 0.8010806 | 1.0183214 | 1.0150082 | 1.0313096 | 1.0486721 | 1.30762 |
| Phase-1 RCT-117 | 1.3554323 | 1.4340345 | 1.2542573 | 1.2450678 | 0.9199008 | 0.5740656 | 1.3832319 |
| Phase-1 RCT-102 | 1.2307757 | 1.9138232 | 0.9119279 | 0.967006 | 0.7442403 | 0.9762455 | 0.5647314 |
| 3-hydroxyisobutyrate dehydrogenase | 0.854547 | 0.9535527 | 0.982198 | 0.9635606 | 0.9837244 | 0.9358741 | 0.959917 |
| ATPase inhibitor (rat mitochondrial IF1 | 0.8521345 | 0.7769733 | 0.9893217 | 0.9580447 | 0.7947021 | 0.9280893 | 0.9236015 |
| protein) | | | | | | | |
| Alpha-2-macroglobulin | 0.5158899 | 0.8243105 | 1.2023191 | 0.9476277 | 1.5486226 | 1.2187929 | 1.3474845 |
| Phase-1 RCT-137 | 0.7058342 | 0.853945 | 0.9997141 | 1.0079024 | 0.936773 | 0.9801365 | 0.9891708 |
| Phase-1 RCT 252 | 1.0286615 | 0.9365324 | 1.1187922 | 1.1234202 | 1.0011952 | 0.9923267 | 0.9527439 |
| Phase-1 RCT-65 | 1.3204659 | 1.3860147 | 1.0607959 | 1.033921 | 1.1916884 | 1.2472743 | 1.0737641 |
| Liver fatty acid binding protein | 0.9839757 | 0.940463 | 1.0134888 | 0.9971262 | 1.0103929 | 1.0122951 | 1.2906444 |
| Carbamyl phosphate synthetase I | 1.0720181 | 0.9905011 | 1.1458106 | 1.1401561 | 1.0332978 | 1.0017059 | 0.943414 |
| c-jun | 1.6492165 | 1.2417426 | 0.9742847 | 1.0464569 | 0.8643965 | 1.6226351 | 1.0218707 |
| Transthyretin | 0.7406973 | 0.763625 | 0.8977307 | 0.8361629 | 0.9019695 | 1.134158 | 0.604907 |
| Aflatoxin B1 aldehyde reductase | 0.6124246 | 0.6169556 | 0.9851978 | 0.9435223 | 1.1376884 | 1.0271837 | 0.8807532 |
| Phase-1 RCT-15 | 1.0957676 | 1.2144754 | 0.980016 | 1.0338566 | 1.18659 | 1.245339 | 0.9508964 |
| Insulin-like growth factor I | 1.1445698 | 1.0134277 | 0.9349118 | 0.8838595 | 0.885825 | 1.1993126 | 0.9160149 |
| Sodium/bile acid cotransporter | 0.787307 | 0.892918 | 1.053679 | 0.9446503 | 1.1681696 | 0.9514156 | 1.31466 |
| Organic cation transporter 3 | 0.9131043 | 0.8597093 | 1.0306678 | 0.997267 | 1.037939 | 1.0350468 | 1.2607527 |
| Gadd153 | 1.0654631 | 1.1022518 | 0.901045 | 0.9596821 | 0.9808468 | 0.9757422 | 1.0279391 |
| Phase-1 RCT-109 | 0.9839159 | 1.090196 | 1.03229 | 1.1243688 | 1.0318047 | 1.1825358 | 1.3653173 |
| Phase-1 RCT-88 | 1.0496689 | 0.9948443 | 0.8534522 | 1.0048211 | 0.7230025 | 0.7174607 | 0.8775012 |
| Ubiquitin conjugating enzyme (RAD 6 | 0.8789052 | 0.9779565 | 1.0707263 | 0.9854753 | 1.019171 | 0.9614476 | 1.2507049 |
| homologue) | | | | | | | |
| Glucose-regulated protein 78 | 1.2133586 | 1.2478733 | 1.1447569 | 0.9169621 | 0.8795071 | 1.0647907 | 1.0190637 |
| Ribosomal protein L13A | 0.9151192 | 1.057748 | 0.986749 | 1.0644746 | 1.0530075 | 1.2076026 | 1.4087931 |
| Monocyte chemotactic protein receptor | 1.0808592 | 1.2179242 | 0.9790533 | 1.0102938 | 0.9992982 | 0.9539036 | 1.1579303 |
| (CCR2) | | | | | | | |
| Fas antigen | 0.9132685 | 0.9702953 | 1.117694 | 1.0470116 | 1.085591 | 1.1050112 | 1.1259136 |
| Calpactin I heavy chain | 1.162256 | 1.0533704 | 1.0363821 | 1.1351584 | 1.0378681 | 1.05581 | 0.9856164 |
| Uncoupling protein 2 | 0.8046101 | 0.9402648 | 1.031474 | 0.9025039 | 1.0278666 | 1.0599056 | 1.239186 |
| Phase-1 RCT-168 | 1.2192364 | 0.9874058 | 1.0990715 | 0.8982461 | 0.9527884 | 0.9388924 | 0.9485305 |
| Phase-1 RCT-154 | 0.9349719 | 1.0264267 | 1.047096 | 1.0404276 | 0.9925371 | 1.0093783 | 1.045577 |
| Superoxide dismutase Mn | 1.055182 | 1.153363 | 1.0530064 | 1.0815418 | 1.09284 | 1.1650927 | 1.3349029 |
| Phase-1 RCT-214 | 1.1935552 | 1.0984516 | 0.8973067 | 0.7897611 | 0.7506299 | 0.85414 | 0.7565825 |
| Phase-1 RCT-225 | 2.0530102 | 1.4692731 | 0.8041396 | 1.2969481 | 1.2647189 | 1.2666693 | 0.5775507 |
| Phase-1 RCT-181 | 1.2872792 | 1.2258081 | 1.0903194 | 1.0777166 | 0.9549649 | 0.9813189 | 1.3537472 |
| Nucleoside diphosphate kinase beta | 0.900477 | 0.9062983 | 1.0158951 | 0.9978303 | 0.9448464 | 0.9605008 | 1.1630542 |
| isoform | | | | | | | |
| Extracellular-signal-regulated kinase 1 | 1.0114549 | 1.1373141 | 0.9784042 | 0.8351526 | 1.0303891 | 0.7088653 | 0.4122137 |
| {Ribosomal protein L6} | 0.9349878 | 0.8170229 | 1.0799739 | 1.0970956 | 1.0638716 | 1.1433125 | 1.2916285 |
| Aldehyde dehydrogenase, microsomal | 0.9807548 | 0.8360473 | 1.197637 | 1.0178883 | 1.0551094 | 1.1900252 | 0.9592723 |
| p53 | 0.7883779 | 0.9708037 | 0.9427902 | 0.941774 | 0.9704472 | 1.0204779 | 0.9648071 |
| Phase-1 RCT-239 | 1.2601181 | 1.0390319 | 1.0439553 | 1.1938931 | 1.2281624 | 1.1204593 | 0.859755 |
| Macrophage inflammatory protein-2 alpha | 1.2822263 | 1.0896603 | 0.9529997 | 0.8827473 | 1.0371416 | 0.9972296 | 1.0656035 |
| IgE binding protein | 1.0562475 | 1.03294 | 1.0480275 | 1.0655068 | 1.0620606 | 1.1066718 | 1.1449454 |
| Phase-1 RCT-205 | 1.0418348 | 0.9223324 | 0.9740601 | 0.9603548 | 0.906594 | 0.9325743 | 0.9885315 |
| Melanoma-associated antigen ME491 | 0.8591662 | 0.8684787 | 0.9450409 | 0.9030111 | 1.026077 | 0.9494096 | 1.1832627 |
| Phase-1 RCT-242 | 1.1349999 | 1.0035763 | 1.0096862 | 1.0796684 | 1.0523583 | 1.1367192 | 0.9255114 |
| Enolase alpha | 0.6003254 | 0.6213133 | 0.9080579 | 1.034633 | 0.9780739 | 1.0650513 | 1.3101355 |
| Thymosin beta-10 | 0.7899596 | 0.9146676 | 1.0686759 | 1.0376713 | 0.9657999 | 1.0929132 | 1.3850416 |
| Cofilin | 0.9111455 | 0.910445 | 0.9537863 | 0.9462809 | 1.0517476 | 1.0217646 | 0.8435281 |
| Carbonyl reductase | 0.8955374 | 1.080439 | 0.9830242 | 0.9616811 | 0.9803597 | 0.866851 | 0.9793747 |
| MAP kinase kinase | 0.8597964 | 0.7909377 | 0.9998791 | 0.9750609 | 1.0751804 | 1.077727 | 0.9679903 |
| Phase-1 RCT-162 | 1.0274882 | 1.0404625 | 1.0155525 | 1.0220867 | 0.9851858 | 1.043331 | 1.2459162 |
| Phase-1 RCT-72 | 1.199637 | 1.099171 | 0.9987163 | 1.1000473 | 1.0170416 | 1.0813669 | 0.9837225 |
| Ribosomal protein S8 | 0.8131328 | 0.8200875 | 1.0516864 | 1.0469987 | 1.0447752 | 1.1236109 | 1.5165348 |
| Proliferating cell nuclear antigen gene | 1.0222557 | 0.9913463 | 0.9087323 | 0.9220129 | 0.9640177 | 0.9312899 | 0.9139877 |
| Phase-1 RCT-192 | 0.9199516 | 1.0867399 | 0.9880838 | 0.9973732 | 0.9433329 | 0.923407 | 1.3487914 |
| IkB-a | 0.753287 | 0.7720562 | 0.9401116 | 1.0043068 | 0.9477188 | 1.0527065 | 1.2303292 |
| Endogenous retroviral sequence, 5′ and | 1.0279377 | 1.1070669 | 0.9789911 | 1.1128079 | 1.1103172 | 0.8919403 | 0.5302711 |
| 3′ LTR | | | | | | | |
| Phase-1 RCT-37 | 1.0713532 | 0.9676918 | 1.0680957 | 1.0543905 | 1.0298688 | 1.0992798 | 1.2211272 |
| Matrix metalloproteinase-1 | 1.0256789 | 0.9979149 | 1.1046586 | 1.0348411 | 1.1515146 | 1.0521729 | 1.3280245 |
| Cyclin G | 1.1436332 | 1.1285464 | 0.985998 | 1.0409845 | 1.0224708 | 1.0816298 | 1.0224284 |
| DNA polymerase beta | 0.8993062 | 0.9665736 | 1.0349258 | 0.9623957 | 0.9987246 | 0.9940012 | 1.2576497 |
| Phase-1 RCT-127 | 0.9060416 | 0.9184349 | 1.0482055 | 1.076837 | 0.9510025 | 0.8766478 | 1.1084449 |
| Multidrug resistant protein-2 | 0.9028021 | 0.9808773 | 0.9169236 | 0.8775058 | 1.1235701 | 1.0640013 | 1.3016158 |
| Ribosomal protein S9 | 0.8269926 | 0.8806605 | 1.0499238 | 0.9388887 | 0.9365688 | 0.9622657 | 1.3325914 |
| Phase-1 RCT-12 | 1.1987983 | 1.2095588 | 0.9499573 | 0.9257831 | 0.9377951 | 1.0229154 | 1.104018 |
| Beta-tubulin, class I | 1.3050863 | 1.3948457 | 1.0409039 | 1.1434577 | 0.9384645 | 1.2530004 | 1.5082289 |
| Argininosuccinate lyase | 1.1676921 | 1.0434597 | 1.230507 | 1.0986362 | 1.2655351 | 1.2319297 | 1.195606 |
| Elongation factor-1 alpha | 0.7997279 | 0.7619379 | 1.002233 | 0.9746004 | 1.1289371 | 1.0316497 | 1.2954711 |
| Phase-1 RCT-55 | 1.2269814 | 1.058236 | 1.0730972 | 1.0626581 | 1.0714684 | 1.2093532 | 1.0887252 |
| Macrophage inflammatory protein-1 alpha | 1.0088532 | 1.1471485 | 1.0644583 | 1.018292 | 0.9844701 | 0.9172412 | 1.048071 |
| c-H-ras | 1.1523035 | 1.0517048 | 1.0857929 | 1.0366333 | 1.07764 | 0.9559768 | 1.3595871 |
| Multidrug resistant protein-1 | 0.8068215 | 0.8465738 | 0.8035657 | 1.0856668 | 0.8326201 | 0.9753419 | 1.1417308 |
| T-cell cyclophilin | 0.7850029 | 0.8296217 | 0.9849263 | 0.9047664 | 0.94337 | 0.9501134 | 1.1976178 |
| Phase-1 RCT-185 | 0.844238 | 0.8708094 | 0.8933179 | 0.9412158 | 0.8763824 | 0.9215216 | 0.8757951 |
| 25-DX | 0.8196313 | 0.8929417 | 1.0839146 | 0.9685113 | 0.9692293 | 1.0123106 | 1.1651486 |
| Ref-1 | 1.2717825 | 1.1691545 | 1.0673232 | 1.0040103 | 1.2007607 | 1.1205279 | 1.3111258 |
| Apolipoprotein CIII | 0.7819864 | 0.7847065 | 1.0563716 | 0.967483 | 1.1154201 | 1.01234 | 0.8177691 |
| NADPH quinone oxidoreductase-1 (DT- | 1.3178068 | 0.9772558 | 1.1348553 | 0.9899554 | 1.2668004 | 1.2082883 | 1.5449922 |
| diaphorase) | | | | | | | |
| Pyruvate kinase, muscle | 1.0502899 | 1.445187 | 1.0758014 | 1.1421782 | 0.9761035 | 1.0551362 | 1.1523137 |
| | KETO | LPS | LPS | LPS | MET | MET | MET |
| Compound-Dose (2) | 80 | 2 | 2 | 2 | 1.3 | 5 | 5 |
| Animal Number (3) | 2236 | 344 | 345 | 346 | 224 | 235 | 236 |
| Liver Toxicity Necrosis Classification (4) | no | no | no | no | no | no | no |
| Gene Name (5) | | | | | | | |
| Phase-1 RCT-78 | 0.9411967 | 0.8254955 | 0.9239322 | 0.7613929 | 0.8727672 | 1.0957514 | 1.0818632 |
| Gamma-actin, cytoplasmic | 1.0273638 | 1.4728116 | 1.2995538 | 1.0561625 | 0.5921603 | 1.5311049 | 0.9793918 |
| Matrin F/G | 1.3312683 | 1.2212411 | 1.0614078 | 1.365672 | 0.6968141 | 0.7712636 | 0.5990965 |
| Zinc finger protein | 0.9288141 | 1.126794 | 1.1159219 | 0.9886028 | 1.5783421 | 1.0158045 | 0.9736506 |
| Cathepsin L, sequence 2 | 1.2793449 | 0.9436717 | 1.5763139 | 0.8969238 | 0.5582418 | 0.9361577 | 0.8831233 |
| Gadd45 | 0.7451912 | 0.8678016 | 1.2772439 | 0.9853247 | 1.1733235 | 0.9063532 | 0.8003675 |
| Phase-1 RCT-144 | 0.9824628 | 1.1146936 | 1.240528 | 0.9166004 | 0.990858 | 1.0794798 | 1.2378452 |
| Phase-1 RCT-145 | 0.9646661 | 1.1258441 | 1.3158165 | 1.0964004 | 3.3741124 | 1.0649536 | 1.0439278 |
| Phase-1 RCT-50 | 0.9610024 | 0.8530907 | 1.0160044 | 0.7938151 | 1.4380368 | 1.4134471 | 1.0527131 |
| Phase-1 RCT-92 | 0.9046626 | 0.5417528 | 0.5924412 | 0.587715 | 0.8068374 | 0.7951143 | 1.1521405 |
| Phase-1 RCT-89 | 0.9525853 | 0.8476391 | 0.729455 | 0.8917716 | 0.6617778 | 0.8559978 | 0.7687878 |
| 14-3-3 zeta | 0.9514167 | 1.3725313 | 1.2150381 | 1.1813891 | 1.2783489 | 1.6396852 | 1.4714059 |
| Dynamin-1 (D100) | 1.034197 | 0.7403345 | 0.6832492 | 0.9022481 | 1.090302 | 0.8539639 | 0.900803 |
| Insulin-like growth factor binding protein 1 | 1.4302044 | 1.0328858 | 1.0168333 | 0.9434177 | 0.979361 | 0.7581065 | 0.5683731 |
| L-gulono-gamma-lactone oxidase | 0.9359677 | 0.6416021 | 0.5949166 | 0.7818844 | 1.021713 | 1.2034278 | 1.0270191 |
| Ornithine decarboxylase | 1.0808917 | 1.2346166 | 1.4650202 | 0.9154282 | 3.1134968 | 1.7955642 | 1.592934 |
| PAR interacting protein | 0.9621708 | 1.04938 | 1.1340226 | 0.9698348 | 0.9768099 | 1.1122458 | 1.1579734 |
| Phase-1 RCT-128 | 1.0104522 | 0.6958925 | 0.4101081 | 0.9064781 | 0.432351 | 0.6726341 | 0.7621971 |
| Phase-1 RCT-180 | 1.1083627 | 1.2298504 | 1.4817644 | 1.1603363 | 1.4875165 | 1.1603202 | 1.360334 |
| Phase-1 RCT-182 | 1.0357832 | 0.4836248 | 0.7833743 | 0.5875388 | 0.5536438 | 1.0262095 | 1.177634 |
| Phase-1 RCT-207 | 1.0710576 | 1.0448158 | 1.0363657 | 1.2255818 | 1.3956457 | 1.114726 | 0.9435023 |
| Phase-1 RCT-213 | 1.0416651 | 1.2849755 | 1.3503848 | 1.2912887 | 1.4054707 | 1.0703679 | 1.0274992 |
| Phase-1 RCT-256 | 1.1567645 | 1.2927977 | 0.7549847 | 1.313329 | 0.3993194 | 0.8345807 | 0.6473118 |
| Phase-1 RCT-258 | 1.0035751 | 1.2591363 | 1.425668 | 1.2713425 | 1.0290152 | 1.0485544 | 1.0507412 |
| Phase-1 RCT-264 | 0.8502617 | 0.4417711 | 0.4308484 | 0.59972 | 0.7184985 | 0.8051495 | 0.8247554 |
| Phase-1 RCT-271 | 1.007926 | 0.7066327 | 0.5722729 | 0.8371499 | 1.0588626 | 0.9144318 | 1.2221122 |
| Phase-1 RCT-288 | 1.068113 | 0.7829393 | 0.7517834 | 0.7685401 | 0.579473 | 0.6069111 | 0.532097 |
| Phase-1 RCT-38 | 1.2306331 | 1.3055242 | 0.7416244 | 1.3059711 | 0.3595772 | 0.803121 | 0.5920125 |
| Phase-1 RCT-39 | 0.9702258 | 1.2521025 | 0.93973 | 1.2001494 | 0.7527101 | 1.0805246 | 0.9224786 |
| Phase-1 RCT-68 | 0.8805682 | 1.3280482 | 1.3968937 | 1.1456852 | 1.0835787 | 1.3294483 | 1.3230464 |
| Phase-1 RCT-33 | 0.9222145 | 0.7369658 | 0.5569693 | 0.9384492 | 0.7056956 | 0.7943181 | 0.7034755 |
| Phase-1 RCT-36 | 0.9250851 | 0.8884996 | 0.8386043 | 1.0073639 | 1.1615802 | 0.9611848 | 0.9218688 |
| 17-beta hydroxysteroid dehydrogenase, | 0.7942803 | 0.3417395 | 0.5865167 | 0.3603554 | 0.4359631 | 0.5061427 | 0.6079323 |
| type 2 | | | | | | | |
| Senescence marker protein-30 | 1.0421358 | 1.1806706 | 1.3999538 | 1.308219 | 0.5501186 | 1.1503042 | 1.0645689 |
| Ribosomal protein S17 | 1.3468559 | 1.6399581 | 1.7048751 | 1.4565141 | 0.4561812 | 0.7498756 | 0.7960113 |
| Phase-1 RCT-83 | 1.0177182 | 0.6824303 | 0.8751018 | 0.7452807 | 0.747953 | 0.9308452 | 0.8353369 |
| Phase-1 RCT-49 | 0.9448738 | 1.1274874 | 1.1517112 | 1.1947464 | 0.8929473 | 0.913071 | 0.8405606 |
| Phase-1 RCT-48 | 0.9163891 | 0.8335745 | 0.6666043 | 0.8318753 | 0.6404746 | 1.1769172 | 1.407454 |
| Phase-1 RCT-40 | 0.9896914 | 0.721992 | 0.8253628 | 0.7845329 | 0.6385354 | 1.0060215 | 0.9103243 |
| Phase-1 RCT-296 | 1.0836494 | 0.1882421 | 0.4292468 | 0.3678446 | 0.5796145 | 0.7051622 | 0.8512613 |
| Phase-1 RCT-295 | 1.1059954 | 1.3598763 | 1.2013805 | 1.2618247 | 0.7091503 | 0.9803652 | 0.932249 |
| Phase-1 RCT-291 | 1.0676751 | 0.7207322 | 0.8665208 | 0.7418923 | 0.7611222 | 1.2703853 | 1.1668905 |
| Phase-1 RCT-270 | 0.8087182 | 0.9421668 | 1.070733 | 1.0937221 | 0.7081872 | 1.0789379 | 1.0980442 |
| Phase-1 RCT-241 | 0.8834444 | 1.4915233 | 3.3077483 | 1.2738243 | 1.0405406 | 1.0550077 | 1.03653 |
| Phase-1 RCT-191 | 1.1671367 | 0.8985956 | 1.2911688 | 0.9005617 | 2.8661225 | 1.1314224 | 1.2840335 |
| Phase-1 RCT-189 | 1.0065894 | 1.0133642 | 0.7740685 | 1.0181948 | 0.7625402 | 1.0040705 | 0.9549007 |
| Phase-1 RCT-179 | 0.9982698 | 1.114418 | 1.1620084 | 0.9898636 | 1.012667 | 0.7507123 | 0.86261 |
| Phase-1 RCT-152 | 1.1633782 | 1.8289889 | 1.3017555 | 1.399048 | 0.6608167 | 0.6882049 | 0.7017215 |
| Phase-1 RCT-123 | 0.9505437 | 0.8306587 | 0.7887797 | 0.9522304 | 1.1606523 | 0.9992367 | 1.1166714 |
| Paraoxonase 1 | 0.9966507 | 0.3891244 | 0.4652269 | 0.4689013 | 0.4821432 | 0.7346454 | 1.0368867 |
| Organic anion transporter 3 | 1.2997395 | 0.8302399 | 0.4150339 | 0.8239205 | 0.8157177 | 1.4590472 | 1.2482497 |
| N-hydroxy-2-acetylaminofluorene | 1.1277404 | 0.6457841 | 0.7961873 | 0.6789879 | 0.6782146 | 0.8216351 | 0.7890428 |
| sulfotransferase (ST1C1) | | | | | | | |
| Integrin beta1 | 1.3853215 | 1.8486841 | 1.6701841 | 1.7924253 | 0.8301882 | 1.1922711 | 0.9456514 |
| Insulin-like growth factor binding protein 3 | 1.0888556 | 0.9264851 | 0.695024 | 0.8068856 | 0.6517385 | 0.7449929 | 0.6710215 |
| ID-1 | 1.0471776 | 0.9633551 | 1.062181 | 0.9264293 | 1.3496708 | 1.3387678 | 1.2842237 |
| Hepatic lipase | 0.7931681 | 0.6323757 | 0.3936759 | 0.5865817 | 0.9655135 | 0.7503839 | 0.9322158 |
| Heme oxygenase | 0.7271075 | 2.5563145 | 2.7468493 | 2.0662985 | 0.89527 | 1.0452855 | 0.8398532 |
| Equilbrative nitrobenzylthioinosine- | 1.1271809 | 0.7316783 | 0.8594266 | 0.8097224 | 0.7779241 | 0.8560177 | 0.7736253 |
| sensitive nucleoside transporter | | | | | | | |
| Epidermal growth factor | 0.8895518 | 1.757575 | 0.9382253 | 1.7298249 | 0.7302508 | 0.7216877 | 0.7894197 |
| c-myc | 0.9662433 | 1.0447882 | 1.4613562 | 0.9881479 | 1.5931368 | 1.427438 | 1.202431 |
| Carbonic anhydrase III | 1.0788672 | 0.217058 | 0.2535324 | 0.4904737 | 0.8490672 | 1.3927325 | 1.4924064 |
| Beta-actin | 1.0602597 | 1.8834116 | 1.61845 | 1.6085961 | 1.7933574 | 1.2144873 | 1.0650387 |
| Bax (alpha) | 1.0530555 | 0.8581607 | 1.7328737 | 0.7996875 | 2.721402 | 1.2327665 | 1.1441972 |
| Alpha 1 - inhibitor III | 0.9526491 | 0.3720186 | 0.31342 | 0.5830388 | 0.8074134 | 0.8753479 | 0.9712917 |
| 60S ribosomal protein L6 | 1.136356 | 1.4519244 | 1.6176236 | 1.3338286 | 0.8550111 | 0.7621096 | 0.8744972 |
| Phase-1 RCT-117 | 1.3353597 | 1.0200652 | 0.8605202 | 1.0662884 | 0.877008 | 1.199213 | 0.8001046 |
| Phase-1 RCT-102 | 0.939251 | 0.4009405 | 0.3899821 | 0.3779532 | 0.8310966 | 1.0672108 | 0.7750928 |
| 3-hydroxyisobutyrate dehydrogenase | 1.0014801 | 0.7897868 | 0.7833957 | 0.8706753 | 0.5160866 | 0.9818588 | 1.0300514 |
| ATPase inhibitor (rat mitochondrial IF1 | 0.9580197 | 0.9986082 | 0.9313905 | 0.9503158 | 0.343275 | 0.3913808 | 0.3944967 |
| protein) | | | | | | | |
| Alpha-2-macroglobulin | 1.087621 | 0.6870446 | 1.3769013 | 1.0140972 | 0.7187366 | 1.0327418 | 1.5593811 |
| Phase-1 RCT-137 | 0.9466451 | 1.048999 | 0.9623534 | 0.9924816 | 0.7138952 | 0.6658886 | 0.8337359 |
| Phase-1 RCT 252 | 1.0347347 | 0.7596645 | 0.6542231 | 0.7379984 | 0.5230569 | 0.8835973 | 0.6442218 |
| Phase-1 RCT-65 | 0.9905066 | 1.1218907 | 1.0545754 | 0.9887072 | 2.4295921 | 1.3433158 | 1.2759155 |
| Liver fatty acid binding protein | 1.151641 | 0.9850848 | 0.820356 | 0.8379638 | 0.4096504 | 0.4787793 | 0.4676111 |
| Carbamyl phosphate synthetase I | 1.102737 | 0.7370341 | 0.6392055 | 0.7128905 | 0.4199837 | 0.9509696 | 0.6613867 |
| c-jun | 1.0708773 | 1.046685 | 0.7730988 | 1.0246736 | 1.574772 | 1.7223917 | 0.9238291 |
| Transthyretin | 0.7905402 | 0.4298186 | 0.4009466 | 0.5395997 | 0.4027902 | 0.7037687 | 0.7350139 |
| Aflatoxin B1 aldehyde reductase | 1.0445576 | 0.906312 | 0.5042672 | 0.9106605 | 0.490316 | 0.636012 | 0.7803657 |
| Phase-1 RCT-15 | 1.0610282 | 0.8141824 | 0.7901816 | 0.830665 | 2.2080643 | 1.3731403 | 1.5135815 |
| Insulin-like growth factor I | 0.9997387 | 0.8631354 | 0.7161098 | 0.7595858 | 0.4781644 | 0.7156452 | 0.6560966 |
| Sodium/bile acid cotransporter | 1.0005252 | 0.5635178 | 0.5625144 | 0.5683583 | 0.5232641 | 0.5911949 | 0.6085194 |
| Organic cation transporter 3 | 1.1350403 | 1.360597 | 1.3816513 | 1.3267756 | 0.890412 | 0.683478 | 0.8021241 |
| Gadd153 | 0.9810714 | 1.2014396 | 2.4945552 | 1.0471916 | 1.7231433 | 1.6605246 | 1.2284554 |
| Phase-1 RCT-109 | 1.1405642 | 2.135116 | 1.3631604 | 1.946111 | 0.5411397 | 0.6538152 | 0.4683566 |
| Phase-1 RCT-88 | 0.9042643 | 0.4220288 | 0.698338 | 0.4701825 | 1.1194781 | 0.8348694 | 1.0923152 |
| Ubiquitin conjugating enzyme (RAD 6 | 1.0951017 | 1.1675658 | 1.1139281 | 1.1265495 | 1.2047318 | 0.8396779 | 0.9552209 |
| homologue) | | | | | | | |
| Glucose-regulated protein 78 | 1.1447374 | 2.4369361 | 1.6220107 | 1.7118503 | 0.2808427 | 0.6070681 | 0.6596328 |
| Ribosomal protein L13A | 1.2567679 | 1.5797532 | 1.4674202 | 1.3034056 | 0.4664945 | 0.5974736 | 0.3588808 |
| Monocyte chemotactic protein receptor | 1.0789024 | 1.3022177 | 1.0131018 | 1.2141243 | 1.447535 | 0.8227649 | 0.821635 |
| (CCR2) | | | | | | | |
| Fas antigen | 1.1101309 | 1.5074538 | 1.5125973 | 1.3790355 | 1.3580158 | 0.9419759 | 0.9161348 |
| Calpactin I heavy chain | 1.0846148 | 1.8050145 | 1.9519485 | 1.6177971 | 1.5929023 | 1.3772173 | 1.3257442 |
| Uncoupling protein 2 | 1.045062 | 1.6147901 | 2.5298028 | 1.5595127 | 1.3226427 | 0.7912367 | 0.5595722 |
| Phase-1 RCT-168 | 0.8554356 | 1.0753123 | 1.2393059 | 1.0547968 | 0.6622268 | 0.9082258 | 0.8524969 |
| Phase-1 RCT-154 | 1.00623 | 1.4086311 | 1.2853568 | 1.3672818 | 1.1884358 | 1.0230782 | 0.8901593 |
| Superoxide dismutase Mn | 1.091882 | 2.3164685 | 2.6711466 | 2.7240653 | 0.7550042 | 1.2154483 | 0.8977744 |
| Phase-1 RCT-214 | 0.6741541 | 1.293367 | 0.9092358 | 1.0604389 | 1.160017 | 0.9678867 | 1.1657137 |
| Phase-1 RCT-225 | 1.1927702 | 0.9649061 | 0.8765585 | 0.906066 | 1.6782684 | 0.8833208 | 0.8486914 |
| Phase-1 RCT-181 | 1.1235317 | 0.7811664 | 0.8986276 | 0.8017877 | 0.7331442 | 0.9260154 | 1.0034314 |
| Nucleoside diphosphate kinase beta | 1.0625386 | 1.526713 | 1.765416 | 1.5130631 | 0.8383544 | 0.9545265 | 1.1035591 |
| isoform | | | | | | | |
| Extracellular-signal-regulated kinase 1 | 1.1034206 | 0.8044817 | 0.8421052 | 0.8136261 | 1.999092 | 1.5726149 | 1.4152886 |
| {Ribosomal protein L6} | 1.1701252 | 1.4209286 | 1.587876 | 1.3318864 | 0.5529717 | 0.6474777 | 0.8034105 |
| Aldehyde dehydrogenase, microsomal | 0.9837828 | 0.9603745 | 0.7961101 | 0.8858515 | 0.8292897 | 1.0506895 | 0.8344786 |
| p53 | 1.0112265 | 1.7164048 | 1.261226 | 1.1170584 | 0.8318278 | 0.9424017 | 0.9529958 |
| Phase-1 RCT-239 | 1.172154 | 0.7300673 | 0.9108157 | 0.7443268 | 1.5721025 | 1.4480371 | 1.1696519 |
| Macrophage inflammatory protein-2 alpha | 1.1011482 | 0.9760901 | 0.9733616 | 0.9259647 | 1.0661966 | 1.5876671 | 1.235048 |
| IgE binding protein | 1.0527028 | 2.2124486 | 3.1090653 | 2.2805612 | 0.8720954 | 1.0250872 | 0.8357931 |
| Phase-1 RCT-205 | 0.8851688 | 1.0622364 | 1.027117 | 1.0319928 | 1.0156358 | 1.0791504 | 1.0305196 |
| Melanoma-associated antigen ME491 | 1.3207452 | 1.9099588 | 1.5208948 | 1.459777 | 1.472059 | 1.245471 | 1.2441281 |
| Phase-1 RCT-242 | 0.9117706 | 0.9335592 | 0.9491 | 1.0268097 | 1.3834871 | 1.1232831 | 1.1423848 |
| Enolase alpha | 1.0946906 | 2.554339 | 1.2153989 | 2.147172 | 0.3956447 | 0.5454978 | 0.9222598 |
| Thymosin beta-10 | 1.1612668 | 1.8280458 | 2.3264608 | 1.7165195 | 0.9093212 | 0.6490445 | 0.5560981 |
| Cofilin | 0.9682946 | 0.8375178 | 1.5935396 | 0.980547 | 1.0232598 | 0.9331037 | 1.1904187 |
| Carbonyl reductase | 0.9639153 | 1.7758193 | 1.1930655 | 0.8513634 | 1.9037108 | 1.2623004 | 1.180303 |
| MAP kinase kinase | 0.9256604 | 0.9680806 | 1.1647916 | 0.9872105 | 0.7973072 | 1.2038002 | 0.915712 |
| Phase-1 RCT-162 | 1.0679582 | 1.0648385 | 1.0760273 | 1.0289019 | 0.831462 | 0.8651258 | 0.9150361 |
| Phase-1 RCT-72 | 0.9883342 | 1.1433805 | 0.7346202 | 1.1214807 | 0.8569255 | 1.3034598 | 1.0624208 |
| Ribosomal protein S8 | 1.1816344 | 1.6358085 | 1.59745 | 1.4606279 | 0.5128742 | 0.6143087 | 0.6758723 |
| Proliferating cell nuclear antigen gene | 0.9187899 | 0.8477805 | 1.3069482 | 0.9597963 | 0.8745524 | 1.4996195 | 1.1412503 |
| Phase-1 RCT-192 | 1.0871835 | 1.7667748 | 1.3559887 | 1.3810961 | 0.8176519 | 0.8787565 | 0.9498519 |
| IkB-a | 1.1220281 | 1.3058561 | 1.3024883 | 1.1695149 | 0.5389004 | 0.8379114 | 1.11295 |
| Endogenous retroviral sequence, 5′ and | 1.2154467 | 1.4978466 | 0.7856484 | 0.9622971 | 1.0718268 | 1.2168823 | 0.8888488 |
| 3′ LTR | | | | | | | |
| Phase-1 RCT-37 | 1.1047446 | 1.2469475 | 1.2070819 | 1.1970166 | 0.9007371 | 0.9732634 | 1.0012033 |
| Matrix metalloproteinase-1 | 1.2292367 | 1.1950434 | 1.5157757 | 1.1382587 | 0.6966027 | 0.8415019 | 0.7343459 |
| Cyclin G | 0.9351869 | 1.3843862 | 1.5579362 | 1.4101044 | 0.8950846 | 1.5431851 | 1.2557242 |
| DNA polymerase beta | 1.1707767 | 1.3880438 | 0.9921571 | 1.3894292 | 0.6469046 | 0.7754713 | 0.9254215 |
| Phase-1 RCT-127 | 1.0850483 | 1.1230849 | 0.9904846 | 1.1741145 | 0.9519019 | 1.0880297 | 0.9499342 |
| Multidrug resistant protein-2 | 1.1212188 | 0.8565629 | 1.5932912 | 0.8610166 | 1.1867409 | 1.8033012 | 0.9915088 |
| Ribosomal protein S9 | 1.1353934 | 0.9819822 | 1.6035484 | 1.1039951 | 1.3778001 | 0.8876553 | 1.0526292 |
| Phase-1 RCT-12 | 0.9025739 | 1.4163115 | 1.0893043 | 1.2443941 | 1.5037912 | 1.2523003 | 1.2832004 |
| Beta-tubulin, class I | 1.1140807 | 1.7557983 | 1.5832543 | 1.4513657 | 1.9022529 | 1.4475887 | 1.4667059 |
| Argininosuccinate lyase | 1.1187153 | 0.7539994 | 1.0056387 | 0.7622004 | 1.0882454 | 1.0053443 | 0.9085006 |
| Elongation factor-1 alpha | 1.0704509 | 0.9965161 | 1.1032604 | 0.9363217 | 0.7147029 | 1.0007594 | 0.9201819 |
| Phase-1 RCT-55 | 1.0174932 | 1.1956986 | 0.7311267 | 0.9871891 | 0.8544344 | 1.3760422 | 1.0047342 |
| Macrophage inflammatory protein-1 alpha | 1.0275595 | 1.050494 | 2.043859 | 1.1299301 | 2.2922368 | 1.1376307 | 1.1153075 |
| c-H-ras | 1.0962723 | 0.924342 | 1.1197183 | 1.0524377 | 2.1570125 | 1.026286 | 0.9479259 |
| Multidrug resistant protein-1 | 1.0683627 | 0.9185286 | 1.424878 | 0.8177196 | 1.2398545 | 1.5903449 | 1.3299587 |
| T-cell cyclophilin | 1.0917401 | 1.4455498 | 1.3087262 | 1.3142674 | 0.4395487 | 0.7107895 | 0.9379055 |
| Phase-1 RCT-185 | 0.9645327 | 0.5719068 | 0.5531281 | 0.5993927 | 0.781288 | 0.8939484 | 1.317733 |
| 25-DX | 1.0122457 | 1.194328 | 0.6316726 | 1.1202568 | 0.520195 | 0.6597415 | 0.6251457 |
| Ref-1 | 1.0176699 | 1.155659 | 0.8982155 | 1.3489524 | 0.9649477 | 0.9757319 | 0.7613632 |
| Apolipoprotein CIII | 0.9125352 | 0.9999999 | 0.8101848 | 1.020899 | 1.178826 | 0.9145267 | 1.022237 |
| NADPH quinone oxidoreductase-1 (DT- | 1.0267385 | 0.8676032 | 1.4956099 | 1.4191957 | 1.5116162 | 0.9379379 | 0.6922599 |
| diaphorase) | | | | | | | |
| Pyruvate kinase, muscle | 1.1012504 | 0.8956803 | 0.6958787 | 1.095069 | 1.3910917 | 1.1413805 | 1.0162992 |
| | NAL | NAL | NAL | NAL | NAL | NAL | PBARB |
| Compound-Dose (2) | 45 | 45 | 45 | 180 | 180 | 180 | 20 |
| Animal Number (3) | 2644 | 2645 | 2646 | 2654 | 2655 | 2656 | 2624 |
| Liver Toxicity Necrosis Classification (4) | no | no | no | no | no | no | no |
| Gene Name (5) | | | | | | | |
| Phase-1 RCT-78 | 1.2040777 | 1.1066562 | 1.1756521 | 1.1273028 | 1.3172523 | 1.8338281 | 1.0189993 |
| Gamma-actin, cytoplasmic | 1.1214224 | 1.1122547 | 0.7108213 | 1.2212256 | 1.0807403 | 1.088423 | 1.0630708 |
| Matrin F/G | 0.942437 | 0.9641721 | 0.9139816 | 0.936215 | 0.9888864 | 0.8888976 | 1.0345274 |
| Zinc finger protein | 0.9737642 | 0.8482391 | 0.8579915 | 0.7035312 | 0.9778501 | 0.945248 | 1.0421536 |
| Cathepsin L, sequence 2 | 1.2560879 | 1.1263744 | 1.013816 | 0.6900481 | 1.196563 | 1.2324036 | 1.2727754 |
| Gadd45 | 1.0296901 | 0.8624209 | 1.317923 | 1.2205976 | 1.0909567 | 1.1962068 | 0.9408175 |
| Phase-1 RCT-144 | 0.9882849 | 1.0551181 | 0.9866477 | 0.9887608 | 1.0962118 | 0.9948188 | 0.9884214 |
| Phase-1 RCT-145 | 0.9042199 | 0.829957 | 0.8521531 | 0.9821643 | 1.0992682 | 1.0161685 | 1.0392357 |
| Phase-1 RCT-50 | 0.8135692 | 0.7998242 | 0.7442276 | 0.7796 | 0.8438841 | 0.8072372 | 0.8834606 |
| Phase-1 RCT-92 | 1.149176 | 1.4315958 | 1.1992453 | 1.1335571 | 1.0664116 | 0.9077645 | 1.203757 |
| Phase-1 RCT-89 | 1.1475078 | 1.0847466 | 1.2382659 | 1.2584554 | 1.1479499 | 0.9699952 | 1.1533846 |
| 14-3-3 zeta | 0.8954953 | 1.366843 | 0.9702546 | 0.7700481 | 0.755591 | 1.1659337 | 0.908553 |
| Dynamin-1 (D100) | 0.9697427 | 1.2835578 | 1.1936505 | 0.9103717 | 0.87366 | 0.8466398 | 0.9340932 |
| Insulin-like growth factor binding protein 1 | 1.0095598 | 0.9242203 | 0.8998103 | 0.9143368 | 0.9048118 | 1.121623 | 1.2334747 |
| L-gulono-gamma-lactone oxidase | 0.8472056 | 0.9082857 | 1.1384842 | 0.7283352 | 0.6586729 | 0.5415166 | 1.0285842 |
| Ornithine decarboxylase | 1.0591656 | 1.3108741 | 0.954625 | 1.2021352 | 1.2125236 | 1.1312973 | 1.1725769 |
| PAR interacting protein | 0.8987401 | 0.9360948 | 0.8200094 | 0.9944221 | 1.0216209 | 0.9518735 | 1.0961226 |
| Phase-1 RCT-128 | 1.0572059 | 1.4739848 | 1.3877952 | 1.0644243 | 1.0164059 | 0.8329592 | 0.8580686 |
| Phase-1 RCT-180 | 1.3672848 | 1.2404883 | 1.2443647 | 1.1696856 | 1.1577052 | 1.1910163 | 1.2780552 |
| Phase-1 RCT-182 | 1.2754034 | 1.3091512 | 1.189788 | 1.3530902 | 1.4228779 | 0.9246029 | 1.2028279 |
| Phase-1 RCT-207 | 0.8303126 | 1.0156746 | 0.8516095 | 0.8317751 | 0.9490099 | 0.9746018 | 0.9504951 |
| Phase-1 RCT-213 | 0.9713877 | 0.9436655 | 0.8655031 | 1.1343602 | 1.1624696 | 1.1199952 | 1.1146247 |
| Phase-1 RCT-256 | 0.9401882 | 1.0421045 | 1.4599447 | 1.096916 | 1.0323867 | 0.9262251 | 1.1240284 |
| Phase-1 RCT-258 | 1.0062046 | 0.965409 | 0.9188482 | 1.1367035 | 1.2460519 | 1.0930475 | 1.0931863 |
| Phase-1 RCT-264 | 1.2209013 | 1.1306636 | 1.6571156 | 1.3102388 | 1.0063114 | 0.9853026 | 0.7567725 |
| Phase-1 RCT-271 | 0.9974011 | 1.138257 | 1.3056396 | 1.0356433 | 1.0519078 | 0.8565979 | 1.688782 |
| Phase-1 RCT-288 | 1.2054905 | 1.1624815 | 0.7412354 | 0.8171967 | 0.8003509 | 0.9957246 | 0.8639879 |
| Phase-1 RCT-38 | 0.8991927 | 1.0735395 | 1.4172268 | 1.0950885 | 1.0380607 | 0.9196034 | 1.1550723 |
| Phase-1 RCT-39 | 0.8594564 | 0.617879 | 0.5853301 | 0.718041 | 0.7123396 | 0.8869121 | 0.9373956 |
| Phase-1 RCT-68 | 0.9579059 | 0.9822821 | 0.9283708 | 1.0328423 | 0.7023249 | 1.0500108 | 1.0722576 |
| Phase-1 RCT-33 | 0.8686333 | 0.8334676 | 1.1032861 | 1.0516772 | 1.0073537 | 0.824829 | 1.1247766 |
| Phase-1 RCT-36 | 0.9607282 | 0.9194928 | 0.9675489 | 0.9583876 | 0.9630429 | 0.8874386 | 1.0905216 |
| 17-beta hydroxysteroid dehydrogenase, | 1.0677391 | 0.5138291 | 1.7648649 | 0.7383645 | 0.6386949 | 0.9241032 | 0.8833957 |
| type 2 | | | | | | | |
| Senescence marker protein-30 | 1.2954389 | 1.0175261 | 1.4554753 | 1.1735932 | 0.9330271 | 1.3021619 | 1.058491 |
| Ribosomal protein S17 | 1.1223109 | 1.0160689 | 1.2494246 | 1.5047197 | 1.7132055 | 2.0056334 | 1.2378737 |
| Phase-1 RCT-83 | 0.9825597 | 0.8528011 | 0.6389619 | 0.6193993 | 0.8094037 | 0.951506 | 1.0637463 |
| Phase-1 RCT-49 | 0.8301092 | 0.8740864 | 0.7945655 | 0.8209793 | 0.8658224 | 0.8512035 | 0.8798802 |
| Phase-1 RCT-48 | 1.1981609 | 1.2058337 | 1.0054092 | 1.2605044 | 1.4583944 | 1.4294807 | 1.1949213 |
| Phase-1 RCT-40 | 1.2691872 | 1.2122601 | 1.0793378 | 1.001851 | 1.3385983 | 1.1642632 | 1.0714364 |
| Phase-1 RCT-296 | 1.4171555 | 1.3164165 | 1.4291129 | 1.042626 | 1.0991402 | 0.9029802 | 1.0508249 |
| Phase-1 RCT-295 | 1.0999447 | 1.0770676 | 1.079925 | 1.2125319 | 1.285039 | 1.1896448 | 1.3035039 |
| Phase-1 RCT-291 | 1.2674195 | 1.1148016 | 1.237545 | 1.1183105 | 0.9517651 | 0.9876375 | 1.4814453 |
| Phase-1 RCT-270 | 1.1378872 | 1.1807256 | 0.9980024 | 1.2422948 | 1.1392775 | 1.0750597 | 0.9538332 |
| Phase-1 RCT-241 | 0.9131725 | 0.7324926 | 0.6691209 | 0.7310898 | 0.7531202 | 0.9310862 | 1.0452049 |
| Phase-1 RCT-191 | 0.8811861 | 1.0745221 | 0.8401624 | 1.0542101 | 1.0555668 | 0.9455711 | 1.2291837 |
| Phase-1 RCT-189 | 1.0298295 | 1.2210543 | 1.2686024 | 0.9871813 | 0.8433522 | 0.9631251 | 0.9670175 |
| Phase-1 RCT-179 | 1.0474746 | 1.1483874 | 0.8484138 | 0.7390721 | 0.8049483 | 1.0659615 | 1.0935851 |
| Phase-1 RCT-152 | 1.0821545 | 1.0314994 | 0.9518418 | 1.2400241 | 1.3075821 | 1.4334339 | 0.9807447 |
| Phase-1 RCT-123 | 0.997732 | 1.0705374 | 0.9051793 | 0.6986835 | 0.857298 | 0.8840246 | 0.9725327 |
| Paraoxonase 1 | 1.3232759 | 1.2379159 | 1.2734405 | 1.1374274 | 1.1692327 | 0.8875405 | 1.0000651 |
| Organic anion transporter 3 | 0.9998856 | 0.5754557 | 0.5826224 | 0.7021413 | 0.7211879 | 0.6458275 | 1.1219102 |
| N-hydroxy-2-acetylaminofluorene | 1.1226163 | 0.9798814 | 0.9148696 | 0.5723575 | 1.0113006 | 0.880556 | 1.0506246 |
| sulfotransferase (ST1C1) | | | | | | | |
| Integrin beta1 | 0.9371607 | 0.9087053 | 0.8386258 | 0.9417765 | 0.923899 | 1.0868127 | 0.9471425 |
| Insulin-like growth factor binding protein 3 | 1.1007221 | 0.9121733 | 1.0930117 | 1.2661333 | 0.9789984 | 1.2083311 | 0.9636385 |
| ID-1 | 0.8091428 | 0.8462172 | 0.9398953 | 1.0058473 | 1.0058315 | 0.8770729 | 0.8844202 |
| Hepatic lipase | 0.9032075 | 1.1485763 | 1.11714 | 0.7437439 | 0.8021107 | 0.5170425 | 1.1308197 |
| Heme oxygenase | 1.1155461 | 0.9021917 | 1.315695 | 1.5988879 | 1.8892936 | 2.8961155 | 1.0067487 |
| Equilbrative nitrobenzylthioinosine- | 1.0200003 | 0.8633021 | 0.8475984 | 0.4989112 | 1.0098784 | 0.9443255 | 1.0007473 |
| sensitive nucleoside transporter | | | | | | | |
| Epidermal growth factor | 0.794392 | 0.7347957 | 0.9506928 | 0.9656344 | 0.9871793 | 0.8914056 | 0.9036452 |
| c-myc | 0.7665244 | 0.7922169 | 0.9845398 | 1.0128802 | 0.9801688 | 1.0196549 | 0.8047984 |
| Carbonic anhydrase III | 1.31315 | 1.3088467 | 1.2987891 | 0.2896877 | 1.5152373 | 0.7801934 | 1.1920874 |
| Beta-actin | 0.9065251 | 1.4832731 | 0.7401255 | 1.1042328 | 0.8703842 | 1.0226983 | 0.7757172 |
| Bax (alpha) | 0.8543482 | 0.8423131 | 0.8780714 | 1.1074398 | 1.1316482 | 1.026579 | 0.9488975 |
| Alpha 1 - inhibitor III | 0.7824219 | 1.3584509 | 1.541396 | 1.0183612 | 0.7721893 | 0.758899 | 0.6585815 |
| 60S ribosomal protein L6 | 1.0753756 | 1.1603794 | 1.3051459 | 1.4780642 | 1.548892 | 1.7556303 | 1.1305933 |
| Phase-1 RCT-117 | 1.6276275 | 1.5523903 | 1.4595478 | 1.3799983 | 1.7329228 | 1.1531007 | 1.3582462 |
| Phase-1 RCT-102 | 0.8447 | 0.6427715 | 0.7889429 | 0.6539396 | 0.6700307 | 0.7161397 | 0.8732526 |
| 3-hydroxyisobutyrate dehydrogenase | 1.1284628 | 1.1797142 | 1.3591552 | 1.2215188 | 1.2890364 | 1.0882375 | 1.1856974 |
| ATPase inhibitor (rat mitochondrial IF1 | 0.9639291 | 0.9285457 | 1.1396357 | 0.7734738 | 0.7437181 | 1.1355599 | 0.9077637 |
| protein) | | | | | | | |
| Alpha-2-macroglobulin | 0.7832562 | 0.7166321 | 0.9103125 | 0.6992743 | 0.7524802 | 0.7891514 | 0.6469988 |
| Phase-1 RCT-137 | 1.1094037 | 1.2695209 | 1.4849468 | 1.2344115 | 0.9999999 | 0.9990186 | 1.306499 |
| Phase-1 RCT 252 | 0.8311172 | 1.430933 | 1.483369 | 1.0309699 | 1.095501 | 0.7363722 | 1.0577892 |
| Phase-1 RCT-65 | 0.936739 | 0.953924 | 0.9886985 | 1.1371061 | 0.9506738 | 1.0763657 | 0.9634923 |
| Liver fatty acid binding protein | 1.2681334 | 1.0337187 | 1.065481 | 1.4900931 | 1.0102762 | 1.2340068 | 1.2483753 |
| Carbamyl phosphate synthetase I | 0.8364756 | 1.4792482 | 1.5581411 | 1.0632536 | 1.1016436 | 0.6948665 | 0.9366696 |
| c-jun | 0.8424038 | 0.6543116 | 1.001915 | 0.7951392 | 0.7339309 | 0.703728 | 0.8303393 |
| Transthyretin | 1.0163106 | 0.9509039 | 1.3015764 | 1.095263 | 0.8814501 | 0.8234068 | 0.8017502 |
| Aflatoxin B1 aldehyde reductase | 1.2886465 | 1.2152185 | 1.9831855 | 7.698345 | 4.8343053 | 3.2279685 | 1.0789213 |
| Phase-1 RCT-15 | 1.200953 | 1.2619003 | 1.3040715 | 1.2635123 | 1.1249137 | 1.0329844 | 1.2607615 |
| Insulin-like growth factor I | 1.1987956 | 1.1169683 | 1.0069631 | 1.2579076 | 0.9074423 | 1.2722701 | 0.8484668 |
| Sodium/bile acid cotransporter | 1.4807682 | 1.19271 | 1.4264773 | 1.2560571 | 1.5503533 | 1.5445105 | 1.3746221 |
| Organic cation transporter 3 | 1.0286256 | 1.0891267 | 1.2533561 | 1.426678 | 1.5446063 | 1.7307856 | 1.1140827 |
| Gadd153 | 0.8167011 | 0.7948633 | 0.9880104 | 0.9995397 | 1.0336845 | 0.9757027 | 0.8928386 |
| Phase-1 RCT-109 | 0.8863757 | 1.0233607 | 1.0459566 | 1.1912588 | 1.2338687 | 1.3265096 | 1.1086216 |
| Phase-1 RCT-88 | 1.1496282 | 1.57613 | 1.1861619 | 1.1855584 | 1.07521 | 1.1038438 | 1.0623679 |
| Ubiquitin conjugating enzyme (RAD 6 | 1.2427821 | 1.2129275 | 1.407106 | 1.2454127 | 1.347129 | 1.3363819 | 1.3152463 |
| homologue) | | | | | | | |
| Glucose-regulated protein 78 | 0.9707059 | 1.0328547 | 1.1868162 | 1.6645918 | 1.1081907 | 1.4262978 | 1.2076089 |
| Ribosomal protein L13A | 0.7977811 | 0.9529903 | 1.0370792 | 1.163307 | 1.3045912 | 1.3455061 | 1.0750833 |
| Monocyte chemotactic protein receptor | 0.6199161 | 0.6669797 | 0.7380988 | 0.8888436 | 0.9031825 | 1.1273327 | 1.0818825 |
| (CCR2) | | | | | | | |
| Fas antigen | 0.9824907 | 1.0708266 | 0.8516111 | 1.0413183 | 0.9363588 | 1.1832144 | 0.8644142 |
| Calpactin I heavy chain | 1.0851088 | 0.884501 | 0.9650977 | 1.228202 | 1.4323419 | 1.757792 | 1.0267892 |
| Uncoupling protein 2 | 0.8721341 | 0.897956 | 0.8748241 | 0.9448691 | 1.1055139 | 1.3031064 | 0.9370846 |
| Phase-1 RCT-168 | 0.9106722 | 1.0111204 | 1.2298381 | 1.2343104 | 1.1066872 | 1.072505 | 1.2174448 |
| Phase-1 RCT-154 | 1.0732797 | 0.8653848 | 0.9272546 | 0.9818855 | 1.1567302 | 1.2157241 | 1.1096146 |
| Superoxide dismutase Mn | 1.2815182 | 1.3424653 | 1.1920233 | 1.6315763 | 1.7596872 | 1.4134419 | 1.1765287 |
| Phase-1 RCT-214 | 1.0745115 | 1.0002277 | 0.8652571 | 0.7788431 | 0.7458316 | 1.0481452 | 1.2373424 |
| Phase-1 RCT-225 | 0.9843891 | 1.1548651 | 0.6649586 | 0.8526075 | 0.5611728 | 2.0515995 | 1.1320049 |
| Phase-1 RCT-181 | 1.0114653 | 1.2563285 | 1.1405444 | 1.3653687 | 1.0297879 | 0.9294831 | 1.1221112 |
| Nucleoside diphosphate kinase beta | 0.9072705 | 0.6807867 | 0.749705 | 0.7933139 | 0.9347583 | 1.2246327 | 1.0655922 |
| isoform | | | | | | | |
| Extracellular-signal-regulated kinase 1 | 1.3172253 | 1.3347229 | 1.3157899 | 0.4492676 | 1.2577969 | 0.8215997 | 1.3181909 |
| {Ribosomal protein L6} | 1.0496186 | 1.1171387 | 1.18196 | 1.4039899 | 1.4246587 | 1.6792861 | 1.1006337 |
| Aldehyde dehydrogenase, microsomal | 1.1562272 | 1.1962495 | 1.2392752 | 1.5086234 | 1.3298086 | 1.1998986 | 1.2720841 |
| p53 | 1.0663928 | 0.9859852 | 1.0284336 | 1.0344322 | 1.0596927 | 0.9446959 | 1.0366628 |
| Phase-1 RCT-239 | 0.9199755 | 0.8521722 | 0.7909793 | 0.8795924 | 0.854141 | 0.7371743 | 0.7560803 |
| Macrophage inflammatory protein-2 alpha | 0.978371 | 0.8416621 | 0.8784871 | 1.0076622 | 1.2191896 | 1.0553563 | 1.0797799 |
| IgE binding protein | 0.9360583 | 0.8334122 | 0.8814431 | 1 | 1.5603224 | 1.9054799 | 0.9299123 |
| Phase-1 RCT-205 | 1.0286695 | 1.0366718 | 0.8817756 | 0.7715429 | 0.8388684 | 0.8612502 | 1.1250507 |
| Melanoma-associated antigen ME491 | 1.0607845 | 0.8826919 | 1.3095828 | 0.9572113 | 1.2969872 | 1.3903663 | 1.1284412 |
| Phase-1 RCT-242 | 0.8579823 | 0.7470564 | 0.7908959 | 0.7499194 | 0.8338187 | 0.7590649 | 0.8921382 |
| Enolase alpha | 1.0306414 | 1.2615392 | 1.1620915 | 1.3161278 | 1.1584475 | 1.4891477 | 1.023046 |
| Thymosin beta-10 | 0.8620089 | 0.9149761 | 1.0111797 | 1.1549972 | 1.3219199 | 1.3810022 | 1.0041044 |
| Cofilin | 1.3170716 | 1.3227862 | 1.3518732 | 1.3017913 | 1.2432821 | 1.2494075 | 0.901139 |
| Carbonyl reductase | 0.9139171 | 0.8622263 | 1.0062319 | 1.0359313 | 0.9557377 | 0.9855197 | 0.9584668 |
| MAP kinase kinase | 0.9566995 | 0.758714 | 1.0216486 | 0.9783675 | 0.7860438 | 1.0873249 | 1.0513394 |
| Phase-1 RCT-162 | 1.185978 | 1.1341732 | 1.2258478 | 1.3873429 | 1.6265342 | 1.5402277 | 1.1495726 |
| Phase-1 RCT-72 | 0.8173763 | 0.7237371 | 0.7983726 | 0.828669 | 0.8570422 | 0.9063326 | 1.4427398 |
| Ribosomal protein S8 | 1.1599028 | 1.0838451 | 1.2599728 | 1.4321334 | 1.6455462 | 1.7808539 | 1.3200043 |
| Proliferating cell nuclear antigen gene | 0.8316096 | 0.8405975 | 0.9673021 | 0.9317301 | 0.9168872 | 1.0614325 | 0.9847063 |
| Phase-1 RCT-192 | 0.9585826 | 0.8674195 | 0.8269153 | 0.9937651 | 1.1137217 | 1.5941589 | 1.0062605 |
| IkB-a | 1.0357397 | 1.2159499 | 1.1714486 | 1.1378547 | 1.1198332 | 1.2941346 | 1.0903168 |
| Endogenous retroviral sequence, 5′ and | 1.0623314 | 0.9016954 | 0.5655235 | 0.628648 | 0.6663877 | 1.8134204 | 0.8051625 |
| 3′ LTR | | | | | | | |
| Phase-1 RCT-37 | 1.0917883 | 1.0430442 | 1.087758 | 1.1479757 | 1.2314795 | 1.2371916 | 1.0943716 |
| Matrix metalloproteinase-1 | 1.1620388 | 1.1429127 | 1.059155 | 1.3702747 | 1.5535645 | 1.5094713 | 1.1703331 |
| Cyclin G | 0.8915258 | 0.8468568 | 0.799477 | 0.9966421 | 1.1879214 | 1.0459999 | 0.9731049 |
| DNA polymerase beta | 1.1277554 | 1.0524578 | 1.1774316 | 1.2046107 | 1.2198359 | 1.4323899 | 1.0827327 |
| Phase-1 RCT-127 | 0.9080498 | 0.9510114 | 0.9019446 | 0.8796764 | 0.9779105 | 0.8939804 | 1.0378404 |
| Multidrug resistant protein-2 | 1.1006029 | 1.0964111 | 0.8294478 | 1.4473153 | 1.4696288 | 1.0388944 | 0.9187673 |
| Ribosomal protein S9 | 1.3150295 | 1.2541131 | 1.6367985 | 1.309577 | 1.4895593 | 1.4182341 | 1.2802167 |
| Phase-1 RCT-12 | 0.995904 | 1.0418619 | 0.9246583 | 1.1237488 | 1.0929352 | 1.1073791 | 1.1336422 |
| Beta-tubulin, class I | 1.0954233 | 1.2269781 | 1.0134143 | 1.5483613 | 1.3095841 | 1.3349802 | 1.4159864 |
| Argininosuccinate lyase | 1.0456018 | 1.256429 | 1.1505351 | 1.3509879 | 1.2208346 | 0.9742242 | 0.8459808 |
| Elongation factor-1 alpha | 1.1051992 | 1.1833915 | 1.2256874 | 1.3377715 | 1.4105369 | 1.3935632 | 0.9933733 |
| Phase-1 RCT-55 | 0.9406915 | 0.7982703 | 0.6121365 | 0.6918559 | 0.798774 | 0.8298906 | 0.9645696 |
| Macrophage inflammatory protein-1 alpha | 1.131934 | 1.1142359 | 0.9999999 | 0.7627993 | 0.7743812 | 0.9049805 | 0.9422903 |
| c-H-ras | 0.9440107 | 0.9597198 | 0.8715734 | 1.1644638 | 1.2457188 | 1.4056188 | 1.0461475 |
| Multidrug resistant protein-1 | 1.1135201 | 1.0973839 | 0.8513599 | 1.3866185 | 1.7795007 | 1.0307683 | 0.9737257 |
| T-cell cyclophilin | 1.2196554 | 1.1013584 | 1.224077 | 1.3089522 | 1.357814 | 1.5903109 | 1.0668137 |
| Phase-1 RCT-185 | 1.1679311 | 1.0291506 | 1.3044285 | 0.8548406 | 0.9387766 | 0.7681146 | 0.9021484 |
| 25-DX | 1.1903483 | 1.0926013 | 1.3845651 | 1.1209432 | 1.1776304 | 1.4695295 | 0.9767329 |
| Ref-1 | 1.0395484 | 1.183531 | 1.1496117 | 1.0214565 | 1.265418 | 1.1446743 | 1.2164699 |
| Apolipoprotein CIII | 1.2500995 | 1.0468013 | 1.1350039 | 1.2161075 | 1.5448645 | 1.2730893 | 0.8883826 |
| NADPH quinone oxidoreductase-1 (DT- | 1.3807113 | 1.1966118 | 1.3246766 | 1.0690687 | 2.1697483 | 1.7980651 | 1.1425916 |
| diaphorase) | | | | | | | |
| Pyruvate kinase, muscle | 1.0970548 | 1.0047013 | 0.8936856 | 1.0719582 | 1.177124 | 1.158289 | 1.0986661 |
| | PBARB | PBARB | PBARB | PBARB | PBARB | PHEN | PHEN |
| Compound-Dose (2) | 20 | 20 | 80 | 80 | 80 | 20 | 20 |
| Animal Number (3) | 2625 | 2626 | 2634 | 2635 | 2636 | 1324 | 1325 |
| Liver Toxicity Necrosis Classification (4) | no | no | no | no | no | no | no |
| Gene Name (5) | | | | | | | |
| Phase-1 RCT-78 | 0.9527652 | 0.8618795 | 0.9855703 | 1.1098872 | 0.9080046 | 1.2916156 | 1.3725007 |
| Gamma-actin, cytoplasmic | 1.0540012 | 0.8811097 | 1.2515458 | 1.1673238 | 1.2074906 | 0.7836027 | 0.7810834 |
| Matrin F/G | 1.1807964 | 1.0736411 | 0.9518694 | 0.9552121 | 1.0035175 | 1.0711569 | 0.9981113 |
| Zinc finger protein | 1.0330644 | 1.0142108 | 0.9857174 | 1.001199 | 0.9839848 | 0.8961131 | 0.8505086 |
| Cathepsin L, sequence 2 | 1.5625039 | 1.3666396 | 1.2709851 | 1.5736364 | 1.6328253 | 1.0453055 | 1.1242719 |
| Gadd45 | 1.009062 | 1.0649011 | 1.043594 | 0.9552409 | 1.022072 | 1.1415498 | 1.490055 |
| Phase-1 RCT-144 | 1.1580844 | 1.0519854 | 1.0780061 | 1.2841011 | 1.1368076 | 0.9528768 | 0.8928523 |
| Phase-1 RCT-145 | 1.1486202 | 1.1423323 | 1.1899508 | 1.2725322 | 1.2371436 | 1.0397196 | 1.0227624 |
| Phase-1 RCT-50 | 1.0032948 | 0.9822273 | 0.9333481 | 0.8976036 | 0.9156543 | 1.2030885 | 1.241478 |
| Phase-1 RCT-92 | 0.955446 | 0.9534016 | 1.1055261 | 1.0319965 | 0.9157224 | 1.276463 | 2.106898 |
| Phase-1 RCT-89 | 1.1540354 | 1.0218977 | 0.9166272 | 0.9010934 | 0.9511349 | 0.8282188 | 0.8261514 |
| 14-3-3 zeta | 1.2080936 | 1.2068856 | 1.3493679 | 1.4388244 | 1.3701601 | 1.281665 | 1.3707178 |
| Dynamin-1 (D100) | 0.8477017 | 0.8321644 | 0.9127508 | 0.9716634 | 0.9326314 | 0.9853064 | 0.8393768 |
| Insulin-like growth factor binding protein 1 | 1.7269878 | 1.3567576 | 1.5138423 | 1.6279509 | 1.4860324 | 1.0560021 | 0.9665231 |
| L-gulono-gamma-lactone oxidase | 1.0505722 | 0.8182574 | 0.6365388 | 0.6401941 | 0.705492 | 1.3008119 | 1.5427924 |
| Ornithine decarboxylase | 1.2529758 | 1.0988969 | 1.2622033 | 1.29426 | 1.1606983 | 1.4789826 | 1.6939523 |
| PAR interacting protein | 1.2537847 | 1.2469721 | 1.1956041 | 1.4318604 | 1.2053457 | 0.9790904 | 0.9269387 |
| Phase-1 RCT-128 | 0.7394319 | 0.7416782 | 0.9320839 | 1.0133567 | 0.9409404 | 0.9879366 | 0.9346485 |
| Phase-1 RCT-180 | 1.2404369 | 1.4168665 | 1.0855331 | 1.2957443 | 1.1864618 | 1.0166707 | 0.9865005 |
| Phase-1 RCT-182 | 0.8987951 | 0.9134315 | 0.9123116 | 1.0668904 | 1.0050188 | 0.6963493 | 0.8836139 |
| Phase-1 RCT-207 | 1.0203738 | 1.0239351 | 1.032711 | 1.0787106 | 1.0413905 | 1.1617455 | 1.0558901 |
| Phase-1 RCT-213 | 1.2154524 | 1.1047294 | 1.2077343 | 1.2547921 | 1.1586827 | 1.2132711 | 1.1358421 |
| Phase-1 RCT-256 | 0.9911421 | 0.9833454 | 0.912678 | 0.9761379 | 0.8601963 | 1.1359812 | 1.0892417 |
| Phase-1 RCT-258 | 1.0761275 | 1.0958142 | 1.0942019 | 1.1273836 | 1.0474206 | 1.0526546 | 0.9966706 |
| Phase-1 RCT-264 | 0.5840685 | 0.8440951 | 0.5788525 | 0.5647147 | 0.59357 | 0.5562363 | 0.6031051 |
| Phase-1 RCT-271 | 1.3719628 | 1.0900314 | 1.204021 | 1.7533638 | 1.1575611 | 1.0579194 | 1.1985884 |
| Phase-1 RCT-288 | 0.6739778 | 0.8515782 | 1.0339714 | 0.9835754 | 0.960811 | 0.8204926 | 0.8068599 |
| Phase-1 RCT-38 | 1.0180225 | 0.9756138 | 0.9521973 | 1.012844 | 0.9680494 | 0.9838886 | 0.9960129 |
| Phase-1 RCT-39 | 1.0983394 | 0.9996666 | 1.6308517 | 1.8762019 | 2.263474 | 0.9896256 | 1.2076687 |
| Phase-1 RCT-68 | 1.0791684 | 1.0797226 | 1.1488955 | 1.0461162 | 1.2605457 | 1.2031827 | 1.506034 |
| Phase-1 RCT-33 | 1.2257789 | 1.4710351 | 1.2412357 | 1.2879101 | 1.2397256 | 1.0504283 | 1.1423209 |
| Phase-1 RCT-36 | 1.2118015 | 1.4414533 | 1.1306161 | 1.1577044 | 1.0856223 | 1.0100074 | 1.1195555 |
| 17-beta hydroxysteroid dehydrogenase, | 1.5814844 | 1.3241456 | 0.7561945 | 0.7908122 | 0.9570503 | 1.0882392 | 2.223002 |
| type 2 | | | | | | | |
| Senescence marker protein-30 | 0.7967488 | 0.7544165 | 0.4601038 | 0.4704822 | 0.6828545 | 0.7312896 | 0.8409734 |
| Ribosomal protein S17 | 1.154914 | 1.1405221 | 1.2911185 | 1.228355 | 1.5196855 | 0.7323662 | 0.6157684 |
| Phase-1 RCT-83 | 0.8874185 | 0.8334737 | 0.7947244 | 0.7773083 | 0.8419039 | 0.566869 | 0.5524988 |
| Phase-1 RCT-49 | 0.9102153 | 0.9443811 | 0.907963 | 0.8350582 | 0.8771705 | 0.8690091 | 0.8084037 |
| Phase-1 RCT-48 | 1.0438403 | 0.8561277 | 0.6406795 | 0.7973092 | 0.7094181 | 0.9843347 | 1.3895514 |
| Phase-1 RCT-40 | 1.2163236 | 0.9709314 | 0.9657371 | 1.0988998 | 1.1096889 | 1.1573234 | 1.1572021 |
| Phase-1 RCT-296 | 1.2469704 | 1.4246466 | 0.8164648 | 1.0451744 | 1.1165332 | 1.0063559 | 1.1497546 |
| Phase-1 RCT-295 | 1.4937894 | 1.2329996 | 1.2634397 | 1.3901396 | 1.3733089 | 1.3736389 | 1.5699555 |
| Phase-1 RCT-291 | 1.4855686 | 1.3302574 | 1.1791489 | 1.2853419 | 1.192021 | 1.0344296 | 1.0943284 |
| Phase-1 RCT-270 | 0.8622422 | 1.0220935 | 0.9343709 | 0.9310574 | 0.9426122 | 0.8026044 | 0.728046 |
| Phase-1 RCT-241 | 1.0585343 | 1.0619383 | 1.2651393 | 1.2932581 | 1.3451 | 1.1097966 | 0.9567293 |
| Phase-1 RCT-191 | 1.5182964 | 1.2914625 | 1.4264293 | 1.5989642 | 1.3647133 | 0.6313621 | 0.7151828 |
| Phase-1 RCT-189 | 0.9605923 | 0.9742981 | 0.8764377 | 0.7540069 | 0.6880555 | 0.7720805 | 0.605258 |
| Phase-1 RCT-179 | 0.8634148 | 0.9703842 | 0.96934 | 1.1633886 | 1.0756105 | 0.8977249 | 0.8341951 |
| Phase-1 RCT-152 | 1.0910673 | 1.0417897 | 1.1113588 | 1.1095283 | 1.2457852 | 0.7359347 | 0.7084752 |
| Phase-1 RCT-123 | 0.870975 | 0.9541368 | 0.9384206 | 0.9371893 | 1.1042272 | 1.0197505 | 1.0697129 |
| Paraoxonase 1 | 0.7913593 | 0.8649714 | 0.7219137 | 0.8315134 | 0.9670215 | 0.4981621 | 0.4752466 |
| Organic anion transporter 3 | 1.2482643 | 1.1317321 | 1.2512108 | 1.1947898 | 1.0530753 | 1.0578291 | 1.2896171 |
| N-hydroxy-2-acetylaminofluorene | 0.9898952 | 0.809077 | 0.7492172 | 0.7954482 | 0.8182194 | 0.5527215 | 0.5091497 |
| sulfotransferase (ST1C1) | | | | | | | |
| Integrin beta1 | 1.2715927 | 1.0917827 | 1.2896156 | 1.3701577 | 1.3345927 | 1.120305 | 1.2398175 |
| Insulin-like growth factor binding protein 3 | 0.8871935 | 0.9985392 | 0.7183796 | 0.6158185 | 0.8746586 | 0.8806644 | 0.8119202 |
| ID-1 | 0.8692716 | 1.0079348 | 1.0151496 | 0.9098281 | 1.0150498 | 1.1567293 | 1.3208392 |
| Hepatic lipase | 0.6997933 | 0.9047116 | 0.7302419 | 1.207551 | 0.6736396 | 0.5441151 | 0.4993025 |
| Heme oxygenase | 0.9438345 | 0.931532 | 0.7676747 | 0.755369 | 0.8648719 | 1.4980029 | 1.9078307 |
| Equilbrative nitrobenzylthioinosine- | 1.0175515 | 0.7905172 | 0.9517739 | 0.8269258 | 0.9012228 | 0.6046265 | 0.569324 |
| sensitive nucleoside transporter | | | | | | | |
| Epidermal growth factor | 0.8760634 | 0.98114 | 1.0033023 | 1.0922527 | 1.1438208 | 0.7579396 | 0.6975468 |
| c-myc | 1.2630084 | 0.7701345 | 1.0558499 | 0.7599509 | 0.7591396 | 1.2349048 | 1.1414587 |
| Carbonic anhydrase III | 0.9769893 | 0.6170292 | 1.5019727 | 0.6861851 | 0.8602003 | 2.0685604 | 2.6036627 |
| Beta-actin | 1.3100652 | 1.0077792 | 1.2239356 | 1.2457637 | 1.1447319 | 1.3180724 | 1.3470939 |
| Bax (alpha) | 1.2040212 | 1.0290688 | 1.0579376 | 1.0912734 | 1.1220024 | 1.1137072 | 1.1146199 |
| Alpha 1 - inhibitor III | 0.5583438 | 0.8149803 | 0.5855542 | 0.4904848 | 0.5621914 | 0.5075451 | 0.4307326 |
| 60S ribosomal protein L6 | 1.3516392 | 1.2023529 | 1.2516034 | 1.30657 | 1.4253544 | 0.8080699 | 0.8097126 |
| Phase-1 RCT-117 | 0.9438115 | 0.70393 | 0.8476138 | 1.1803007 | 0.8656678 | 0.9958698 | 1.4190305 |
| Phase-1 RCT-102 | 0.6239475 | 0.6547211 | 0.7132512 | 0.4782684 | 0.5207973 | 1.0955093 | 0.987624 |
| 3-hydroxyisobutyrate dehydrogenase | 1.0795312 | 1.006253 | 0.9364317 | 1.0115885 | 0.986837 | 0.9142559 | 0.9803374 |
| ATPase inhibitor (rat mitochondrial IF1 | 0.6194758 | 0.7189694 | 0.6847957 | 0.7077478 | 0.6801139 | 0.518629 | 0.4781059 |
| protein) | | | | | | | |
| Alpha-2-macroglobulin | 0.7667816 | 0.9845098 | 0.9112625 | 0.8592977 | 0.9766259 | 0.5648875 | 0.4789511 |
| Phase-1 RCT-137 | 0.7925805 | 1.1141845 | 0.8439035 | 0.9250965 | 0.88174 | 0.4276316 | 0.423915 |
| Phase-1 RCT 252 | 0.9399328 | 1.1537405 | 0.8282207 | 0.7719606 | 0.7404517 | 0.8209646 | 0.7562221 |
| Phase-1 RCT-65 | 1.2357296 | 1.051156 | 1.1214749 | 1.1223944 | 1.09479 | 1.064753 | 1.1693969 |
| Liver fatty acid binding protein | 1.1086895 | 1.4777777 | 0.8038268 | 0.7393221 | 0.8984311 | 0.6904743 | 0.5493966 |
| Carbamyl phosphate synthetase I | 0.7852237 | 0.8920481 | 0.8575127 | 0.7796677 | 0.7691843 | 0.8997234 | 0.8454 |
| c-jun | 0.8724486 | 0.8417327 | 0.6641419 | 0.6214476 | 0.6870558 | 1.1399523 | 1.3843127 |
| Transthyretin | 0.5790386 | 0.7326396 | 0.5527899 | 0.5254505 | 0.5896274 | 0.4630527 | 0.4873097 |
| Aflatoxin B1 aldehyde reductase | 1.3820262 | 0.8232412 | 1.4116328 | 1.7273709 | 1.4401507 | 0.655803 | 0.8283816 |
| Phase-1 RCT-15 | 1.427016 | 1.5887231 | 1.0166945 | 1.0001007 | 1.0269015 | 3.5533648 | 2.982262 |
| Insulin-like growth factor I | 0.7484797 | 0.9326515 | 0.6186446 | 0.520038 | 0.7677358 | 0.8550543 | 0.7943858 |
| Sodium/bile acid cotransporter | 1.6056002 | 1.6194117 | 2.870911 | 2.8062227 | 2.7565486 | 0.6926258 | 0.7428868 |
| Organic cation transporter 3 | 1.2764859 | 1.1839733 | 1.2112213 | 1.2770066 | 1.3597053 | 0.9117112 | 0.8387452 |
| Gadd153 | 1.0129519 | 1.0114967 | 1.0078114 | 0.9890363 | 0.9738436 | 1.0866754 | 1.0849292 |
| Phase-1 RCT-109 | 1.0635175 | 1.181331 | 1.0727324 | 1.0347741 | 1.2090583 | 0.8948181 | 0.9983016 |
| Phase-1 RCT-88 | 0.9629591 | 0.916104 | 1.1676247 | 0.8951314 | 0.8890916 | 1.2805486 | 2.1003003 |
| Ubiquitin conjugating enzyme (RAD 6 | 1.2192388 | 1.207253 | 1.2024257 | 1.253254 | 1.2391468 | 0.776266 | 0.6535198 |
| homologue) | | | | | | | |
| Glucose-regulated protein 78 | 1.2606441 | 1.0593652 | 1.6787739 | 1.6684179 | 1.4870768 | 0.8506348 | 0.7386286 |
| Ribosomal protein L13A | 1.1008143 | 1.1759198 | 1.208638 | 1.1272985 | 1.2992722 | 1.0524868 | 1.023628 |
| Monocyte chemotactic protein receptor | 1.1340827 | 1.1778555 | 1.0336486 | 1.0859468 | 1.0710775 | 1.3001536 | 1.4987804 |
| (CCR2) | | | | | | | |
| Fas antigen | 1.2462257 | 1.2256192 | 1.3306442 | 1.4042194 | 1.3285125 | 1.2004303 | 1.2042142 |
| Calpactin I heavy chain | 1.1010026 | 1.1906888 | 1.1336409 | 1.0631132 | 1.2465878 | 1.0933726 | 1.2321887 |
| Uncoupling protein 2 | 1.0282071 | 0.9917117 | 1.0148361 | 0.8976556 | 1.1502259 | 0.963785 | 0.941632 |
| Phase-1 RCT-168 | 1.1077245 | 1.3561 | 1.0874873 | 1.1549605 | 1.1103178 | 0.8115332 | 0.7780997 |
| Phase-1 RCT-154 | 1.0920738 | 1.1250215 | 1.3025905 | 1.1715695 | 0.9325646 | 1.0604428 | 1.006637 |
| Superoxide dismutase Mn | 1.2513832 | 1.0321606 | 1.2658676 | 1.5203067 | 1.4159875 | 1.0924224 | 1.1141198 |
| Phase-1 RCT-214 | 0.850613 | 1.0209765 | 0.9342451 | 1.4075532 | 1.1105812 | 1.0096425 | 0.8956276 |
| Phase-1 RCT-225 | 1.3363762 | 0.9059564 | 1.6865237 | 0.8131781 | 0.9588674 | 0.9126211 | 1 |
| Phase-1 RCT-181 | 0.9651248 | 0.9555553 | 1.0660114 | 1.0416886 | 0.9888852 | 1.1333236 | 1.5037683 |
| Nucleoside diphosphate kinase beta | 1.2703601 | 1.2078403 | 1.3596091 | 1.2880648 | 1.3621202 | 1.0997982 | 1.1869156 |
| isoform | | | | | | | |
| Extracellular-signal-regulated kinase 1 | 1.1310714 | 0.7365924 | 1.5002258 | 1.0713958 | 0.8668441 | 1.3773502 | 1.8045623 |
| {Ribosomal protein L6} | 1.1304626 | 0.9995245 | 1.1591944 | 1.224765 | 1.3009189 | 1.3681743 | 1.1811236 |
| Aldehyde dehydrogenase, microsomal | 1.1315606 | 1.0342352 | 1.0867573 | 1.1255652 | 1.047859 | 1.1007296 | 0.9445829 |
| p53 | 1.1406015 | 1.1233326 | 1.0489068 | 1.1111171 | 0.9729443 | 1.0674292 | 1.1964879 |
| Phase-1 RCT-239 | 0.9364927 | 0.8651457 | 0.8033848 | 0.5981619 | 0.594977 | 1.2344102 | 1.3399453 |
| Macrophage inflammatory protein-2 alpha | 1.1880233 | 1.0088522 | 1.0933721 | 1.1200843 | 1.1774955 | 1.1319822 | 1.3471737 |
| IgE binding protein | 0.8924658 | 0.9113653 | 0.9373995 | 0.8979887 | 1.0133716 | 1.0982697 | 1.165937 |
| Phase-1 RCT-205 | 1.1245536 | 1.0737144 | 1.0843917 | 1.2458733 | 1.1141752 | 1.0286843 | 0.9720383 |
| Melanoma-associated antigen ME491 | 0.9924978 | 1.1190656 | 1.0891837 | 0.9854227 | 1.0451078 | 0.9841422 | 1.0709833 |
| Phase-1 RCT-242 | 0.9504033 | 0.9136221 | 0.9854527 | 0.8678555 | 0.8933881 | 1.1814326 | 1.2135148 |
| Enolase alpha | 1.2378715 | 0.9827299 | 1.1207004 | 1.1179388 | 1.1485008 | 0.7100564 | 0.7241104 |
| Thymosin beta-10 | 1.0950592 | 1.1065707 | 1.0604261 | 0.9766281 | 1.2117712 | 0.7194878 | 0.7558931 |
| Cofilin | 0.8266225 | 0.9116566 | 0.9113568 | 0.8536531 | 0.862679 | 1.074618 | 0.9685963 |
| Carbonyl reductase | 0.9613457 | 0.9159769 | 1.0586959 | 1.1249353 | 0.96204 | 1.0129995 | 1.0348384 |
| MAP kinase kinase | 1.1329185 | 1.1845737 | 1.0019684 | 1.0839792 | 1.108451 | 1.0230565 | 1.1641792 |
| Phase-1 RCT-162 | 1.1340265 | 1.3327436 | 1.2260895 | 1.1699374 | 1.313342 | 0.7764075 | 0.7335387 |
| Phase-1 RCT-72 | 1.3917104 | 1.3799535 | 0.9030739 | 0.859751 | 0.9622939 | 1.1071669 | 1.1823599 |
| Ribosomal protein S8 | 1.41006 | 1.395677 | 1.3087173 | 1.2999768 | 1.449721 | 0.7171001 | 0.6730538 |
| Proliferating cell nuclear antigen gene | 1.3102598 | 0.9114851 | 1.0072708 | 0.9414672 | 1.1079987 | 1.0890363 | 0.9203098 |
| Phase-1 RCT-192 | 0.9811471 | 0.9229795 | 0.9348046 | 1.0713737 | 0.9487991 | 0.9693588 | 0.9295176 |
| IkB-a | 1.304666 | 0.95995 | 1.0235202 | 1.1032907 | 1.0958737 | 0.8327695 | 0.7684462 |
| Endogenous retroviral sequence, 5′ and | 0.9944769 | 0.7515338 | 1.2096317 | 0.7010698 | 0.7797112 | 1.1968359 | 1.1030474 |
| 3′ LTR | | | | | | | |
| Phase-1 RCT-37 | 1.1314616 | 1.0823206 | 1.1007766 | 1.1375151 | 1.2561727 | 1.2545713 | 1.2485778 |
| Matrix metalloproteinase-1 | 1.1713862 | 1.1644468 | 1.2449391 | 1.1935964 | 1.3547534 | 1.5810665 | 1.8966101 |
| Cyclin G | 1.0887344 | 1 | 1.0018926 | 1.0658569 | 1.0789777 | 1.0604428 | 1.1795504 |
| DNA polymerase beta | 1.0562412 | 1.0337721 | 1.0456877 | 1.107049 | 1.1580282 | 0.6488339 | 0.527503 |
| Phase-1 RCT-127 | 0.9212771 | 0.7568857 | 0.8580447 | 1.0451832 | 0.8784117 | 0.9388483 | 1.174943 |
| Multidrug resistant protein-2 | 1.6348426 | 1.1275113 | 1.3007311 | 1.5555578 | 1.4049084 | 1.2861583 | 1.334272 |
| Ribosomal protein S9 | 1.202473 | 1.3338516 | 1.1023035 | 1.331908 | 1.3280592 | 0.6974438 | 0.6832528 |
| Phase-1 RCT-12 | 1.4516833 | 1.3602393 | 1.170492 | 1.2169627 | 1.3653617 | 1.1255708 | 1.0801247 |
| Beta-tubulin, class I | 1.8414356 | 1.5002012 | 1.5072911 | 1.6184801 | 1.632763 | 1.7295218 | 1.5022422 |
| Argininosuccinate lyase | 0.8847442 | 1.0417303 | 0.8355841 | 0.7680508 | 0.8606141 | 0.876506 | 0.9913912 |
| Elongation factor-1 alpha | 1.1777081 | 1.0293189 | 0.9914386 | 1.1530828 | 1.1802515 | 0.7394381 | 0.806003 |
| Phase-1 RCT-55 | 0.950723 | 0.9175192 | 1.0744718 | 0.9761716 | 1.1140611 | 0.9269818 | 0.8830313 |
| Macrophage inflammatory protein-1 alpha | 1.2904842 | 0.9986079 | 1.0505606 | 0.9938096 | 1.025463 | 1.2028037 | 1.1336586 |
| c-H-ras | 0.9875602 | 1.1026089 | 1.1414586 | 1.1798186 | 1.2669216 | 0.8798827 | 0.7846379 |
| Multidrug resistant protein-1 | 1.5768877 | 1.1856297 | 1.3839233 | 1.458121 | 1.3789705 | 1.2529205 | 1.7073212 |
| T-cell cyclophilin | 1.1197088 | 1.0281165 | 1.0239401 | 1.0757828 | 1.1158453 | 0.5842374 | 0.5048292 |
| Phase-1 RCT-185 | 0.7685127 | 0.7025735 | 0.8155935 | 0.7933003 | 0.7196643 | 0.7862408 | 0.8968077 |
| 25-DX | 1.0716887 | 0.8198422 | 1.0367904 | 1.4789933 | 1.1036875 | 0.8826919 | 0.798276 |
| Ref-1 | 1.4992781 | 1.4210588 | 1.9499682 | 2.2664425 | 2.0695798 | 0.8655755 | 0.9440683 |
| Apolipoprotein CIII | 0.7772111 | 0.7250334 | 0.8009769 | 0.75723 | 0.5994049 | 0.9813084 | 0.8842528 |
| NADPH quinone oxidoreductase-1 (DT- | 1.4663283 | 1.4810566 | 2.1605155 | 2.7178252 | 2.2045162 | 0.8727369 | 0.9138369 |
| diaphorase) | | | | | | | |
| Pyruvate kinase, muscle | 1.3283399 | 1.2356503 | 1.0551441 | 1.1328807 | 1.116781 | 1.0669962 | 1.1577379 |
| | PHEN | PHEN | PHEN | PHEN | PEG | PEG | PEG |
| Compound-Dose (2) | 20 | 80 | 80 | 80 | 5000 | 5000 | 5000 |
| Animal Number (3) | 1326 | 1334 | 1335 | 1336 | 144 | 145 | 146 |
| Liver Toxicity Necrosis Classification (4) | no | no | no | no | no | no | no |
| Gene Name (5) | | | | | | | |
| Phase-1 RCT-78 | 1.3931217 | 0.9482955 | 0.8759292 | 0.9633145 | 1.0770189 | 0.8528817 | 0.9374993 |
| Gamma-actin, cytoplasmic | 0.852407 | 0.7751247 | 1.0170078 | 0.8968741 | 0.6544153 | 0.7898974 | 0.9265812 |
| Matrin F/G | 1.3342508 | 1.005943 | 1.2818294 | 0.8953324 | 0.9875332 | 0.9794986 | 1.1212496 |
| Zinc finger protein | 0.9543369 | 0.7087627 | 0.5893349 | 0.7062179 | 1.187741 | 1.133443 | 1.2241381 |
| Cathepsin L, sequence 2 | 1.0858508 | 0.855082 | 0.8699882 | 0.8156406 | 0.8612172 | 1.0352339 | 1.1032578 |
| Gadd45 | 1.1189228 | 1.6145211 | 2.2344668 | 1.8680197 | 1.2939874 | 0.8794358 | 1.018031 |
| Phase-1 RCT-144 | 0.9189215 | 1.0797089 | 1.1179547 | 0.9418925 | 0.8910266 | 0.9280317 | 0.8768233 |
| Phase-1 RCT-145 | 1.1056138 | 1.5420861 | 1.2157737 | 0.9403247 | 1.0918947 | 0.9606311 | 0.9616987 |
| Phase-1 RCT-50 | 1.1953875 | 1.5447166 | 1.4955015 | 1.4834608 | 1.2872071 | 1.1848814 | 1.1412555 |
| Phase-1 RCT-92 | 0.775857 | 0.5369287 | 0.6388049 | 0.8595871 | 0.8470675 | 0.8005772 | 0.998709 |
| Phase-1 RCT-89 | 0.9332854 | 0.7403539 | 0.9482566 | 0.7265291 | 0.9235927 | 1.0681068 | 1.1811488 |
| 14-3-3 zeta | 1.2247324 | 1.3472115 | 2.0428762 | 1.3079387 | 0.9476872 | 0.8924261 | 0.8327295 |
| Dynamin-1 (D100) | 1.0703322 | 0.7426757 | 0.6774321 | 0.7412157 | 1.0895035 | 0.960108 | 1.1260041 |
| Insulin-like growth factor binding protein 1 | 1.2565777 | 1.0156424 | 1.2182635 | 1.2206529 | 1.0963703 | 1.1381238 | 1.0473195 |
| L-gulono-gamma-lactone oxidase | 1.5688523 | 0.9686887 | 0.8881233 | 0.9248349 | 0.5761963 | 0.3793916 | 0.5432696 |
| Ornithine decarboxylase | 1.9620122 | 2.259693 | 2.0640047 | 2.1664467 | 1.1628684 | 0.8550764 | 0.8346332 |
| PAR interacting protein | 0.9119869 | 0.8351138 | 0.8470702 | 0.8845827 | 0.920735 | 0.8767533 | 0.9265968 |
| Phase-1 RCT-128 | 1.1243243 | 0.652554 | 0.6598765 | 0.7240809 | 0.8582925 | 0.7573915 | 1.017798 |
| Phase-1 RCT-180 | 1.0710053 | 0.9005563 | 0.7310515 | 0.7775768 | 0.9281404 | 0.9323387 | 0.8751745 |
| Phase-1 RCT-182 | 0.6138846 | 0.650215 | 0.7012677 | 0.5995349 | 0.9523336 | 1.1499349 | 1.1110083 |
| Phase-1 RCT-207 | 1.1229446 | 1.5989505 | 1.4324657 | 1.3211188 | 1.1064781 | 1.0537797 | 1.051076 |
| Phase-1 RCT-213 | 1.3416873 | 1.6787869 | 1.5124265 | 1.3987917 | 1.1620291 | 0.984861 | 1.0012627 |
| Phase-1 RCT-256 | 1.1763612 | 1.1059732 | 1.1464818 | 1.088121 | 0.7776016 | 0.8039603 | 0.8659218 |
| Phase-1 RCT-258 | 1.0177028 | 1.2105052 | 0.9009715 | 0.9102606 | 1.1499013 | 1.0764107 | 1.0076262 |
| Phase-1 RCT-264 | 0.6290201 | 0.6506881 | 0.4682972 | 0.7910522 | 0.8768913 | 0.7855412 | 1.0580342 |
| Phase-1 RCT-271 | 1.1208429 | 0.9197381 | 0.7563402 | 0.8139433 | 0.8713576 | 0.7849261 | 0.7767287 |
| Phase-1 RCT-288 | 1.0192975 | 0.7866383 | 0.8168164 | 0.9114533 | 1.1487776 | 1.2266461 | 1.5762904 |
| Phase-1 RCT-38 | 1.1665432 | 0.921813 | 1.4052379 | 0.8311333 | 0.7317744 | 0.7544671 | 0.8742822 |
| Phase-1 RCT-39 | 1.2125387 | 1.4129965 | 2.3039718 | 1.4080751 | 0.9392335 | 0.9781045 | 0.9700981 |
| Phase-1 RCT-68 | 1.3386087 | 1.1950661 | 0.9561259 | 1.2978503 | 1.0494736 | 1.0830935 | 1.1013256 |
| Phase-1 RCT-33 | 1.4592078 | 0.9436566 | 1.1198797 | 1.2215226 | 0.6806648 | 0.734388 | 0.9054492 |
| Phase-1 RCT-36 | 1.194796 | 1.0869293 | 1.1415285 | 1.2738779 | 0.7801197 | 0.8378728 | 0.9138964 |
| 17-beta hydroxysteroid dehydrogenase, | 2.1552596 | 1.2159914 | 1.0275359 | 2.6807446 | 0.4884267 | 0.4430632 | 2.5405722 |
| type 2 | | | | | | | |
| Senescence marker protein-30 | 0.6855018 | 0.523519 | 0.422708 | 0.3930963 | 0.9142655 | 0.7294056 | 0.8626612 |
| Ribosomal protein S17 | 0.7517387 | 0.7726013 | 1.0436355 | 0.926692 | 1.1160074 | 1.0409194 | 1.2538981 |
| Phase-1 RCT-83 | 0.6876273 | 0.5323452 | 0.7315042 | 0.6030696 | 0.8357046 | 0.9528832 | 1.04241 |
| Phase-1 RCT-49 | 0.9182984 | 0.9064066 | 1.0867226 | 1.0382648 | 0.9786388 | 0.9641911 | 0.9683083 |
| Phase-1 RCT-48 | 1.2015344 | 1.1332221 | 0.7035986 | 1.2646375 | 0.9290195 | 0.5991244 | 0.6083315 |
| Phase-1 RCT-40 | 1.2435855 | 0.8971276 | 0.7930887 | 0.8744047 | 0.9082475 | 1.2208556 | 1.3333477 |
| Phase-1 RCT-296 | 0.9218822 | 0.8599396 | 0.6412798 | 0.8505126 | 0.3223849 | 0.734029 | 0.7843578 |
| Phase-1 RCT-295 | 1.7035103 | 1.263572 | 1.2837512 | 1.2953727 | 1.0058327 | 0.9491963 | 1.1573129 |
| Phase-1 RCT-291 | 0.8220951 | 1.0675035 | 1.1379033 | 0.9090218 | 0.7117708 | 0.7457661 | 0.7679189 |
| Phase-1 RCT-270 | 0.812644 | 0.597301 | 0.7563118 | 0.4837586 | 0.9457537 | 1.0822341 | 1.190398 |
| Phase-1 RCT-241 | 0.821336 | 1.0871738 | 0.942003 | 0.8767905 | 1.1864891 | 1.3568008 | 1.0014669 |
| Phase-1 RCT-191 | 0.9665746 | 0.7109276 | 0.731363 | 1.0072421 | 0.8779188 | 0.6999713 | 0.7371354 |
| Phase-1 RCT-189 | 0.9829502 | 0.8940215 | 1.1663336 | 1.2709137 | 1.0007676 | 0.9106336 | 1.006867 |
| Phase-1 RCT-179 | 0.9145662 | 0.6312935 | 0.6676126 | 0.7342489 | 0.9877846 | 0.9443004 | 1.1159607 |
| Phase-1 RCT-152 | 1.0087266 | 0.7058618 | 0.9417084 | 0.8778797 | 0.9833272 | 0.948243 | 1.1866969 |
| Phase-1 RCT-123 | 0.9710618 | 1.1866676 | 1 | 1.0934978 | 1.0745821 | 1.1407896 | 1.0248716 |
| Paraoxonase 1 | 0.3993796 | 0.2276444 | 0.4874812 | 0.281783 | 0.6884713 | 0.7016091 | 0.9301739 |
| Organic anion transporter 3 | 1.3252044 | 0.83891 | 1.3748976 | 0.7738117 | 0.5952773 | 0.76925 | 0.9672881 |
| N-hydroxy-2-acetylaminofluorene | 0.5259118 | 0.3377366 | 0.5087699 | 0.4803006 | 0.9006225 | 1.082015 | 1.1971567 |
| sulfotransferase (ST1C1) | | | | | | | |
| Integrin beta1 | 1.5644319 | 1.7936729 | 1.9792509 | 2.031476 | 1.1542113 | 0.8876593 | 0.923847 |
| Insulin-like growth factor binding protein 3 | 0.7833545 | 0.5436072 | 0.6404775 | 0.5933812 | 0.6544629 | 0.7605571 | 0.6743959 |
| ID-1 | 1.5952716 | 1.3105171 | 1.6738713 | 1.3931935 | 0.8917427 | 0.8216634 | 0.7115907 |
| Hepatic lipase | 0.4501011 | 0.3910347 | 0.4342633 | 0.4401655 | 0.664503 | 0.6244513 | 0.6859726 |
| Heme oxygenase | 2.435522 | 3.2578213 | 2.1374784 | 5.6134152 | 1.323749 | 1.0404551 | 0.858879 |
| Equilbrative nitrobenzylthioinosine- | 0.6594676 | 0.5256147 | 0.6549604 | 0.6092755 | 1.0304985 | 1.0830164 | 0.8868218 |
| sensitive nucleoside transporter | | | | | | | |
| Epidermal growth factor | 0.6076459 | 0.6845575 | 0.5963875 | 0.5774066 | 1.0327164 | 1.1091331 | 1.1040318 |
| c-myc | 0.9603114 | 1.8423061 | 2.0737143 | 1.564699 | 1.286979 | 1.032762 | 1.1537318 |
| Carbonic anhydrase III | 1.8291625 | 0.6579221 | 0.1841041 | 0.5841162 | 0.5692562 | 0.3394526 | 0.8492595 |
| Beta-actin | 1.7217063 | 1.3941528 | 1.4223367 | 1.2098732 | 0.5915952 | 0.5561552 | 0.7088081 |
| Bax (alpha) | 1.3624527 | 1.9108559 | 1.9692987 | 1.8977394 | 1.1718135 | 0.9674365 | 0.826158 |
| Alpha 1 - inhibitor III | 0.4357251 | 0.1857627 | 0.2170262 | 0.3086549 | 0.7085946 | 0.514979 | 0.9713823 |
| 60S ribosomal protein L6 | 0.7384698 | 0.8551989 | 0.7346933 | 0.8274319 | 1.0071105 | 0.9131455 | 0.9945142 |
| Phase-1 RCT-117 | 1.0934793 | 1.1788768 | 0.8549924 | 0.8457238 | 0.9662721 | 1.296158 | 1.1448117 |
| Phase-1 RCT-102 | 0.7389631 | 1.3587829 | 1.1500986 | 0.9065123 | 0.6433005 | 0.5028794 | 0.4734413 |
| 3-hydroxyisobutyrate dehydrogenase | 1.1779946 | 0.758692 | 0.4515231 | 1.0148445 | 1.027396 | 1.1424152 | 1.245396 |
| ATPase inhibitor (rat mitochondrial IF1 | 0.6752467 | 0.4003787 | 0.3883172 | 0.5308691 | 1.2585374 | 1.1614265 | 1.1283952 |
| protein) | | | | | | | |
| Alpha-2-macroglobulin | 0.4019149 | 0.3023162 | 0.3487433 | 0.355226 | 0.9832198 | 0.6803443 | 1.299355 |
| Phase-1 RCT-137 | 0.4968751 | 0.3246517 | 0.2864712 | 0.4473665 | 1.0339828 | 0.8551493 | 0.9925233 |
| Phase-1 RCT 252 | 1.0213399 | 0.6133667 | 0.7657425 | 0.7556202 | 0.9817379 | 0.8081656 | 0.9785787 |
| Phase-1 RCT-65 | 1.1685802 | 1.1608448 | 1.1476623 | 0.9204384 | 1.1733794 | 1.1283009 | 0.9905585 |
| Liver fatty acid binding protein | 0.8940865 | 0.4023915 | 0.5264782 | 0.7138901 | 0.5982145 | 0.8247554 | 0.7763153 |
| Carbamyl phosphate synthetase I | 1.1667702 | 0.6088784 | 0.8822693 | 0.7343233 | 0.9501245 | 0.7745791 | 1.0015072 |
| c-jun | 1.2353556 | 1.5851939 | 2.1555805 | 1.9235353 | 0.9425615 | 0.8991846 | 0.9324527 |
| Transthyretin | 0.5127378 | 0.2126588 | 0.2139658 | 0.3407976 | 0.7236181 | 0.6545037 | 0.9452861 |
| Aflatoxin B1 aldehyde reductase | 1.0777822 | 0.5807305 | 0.5022711 | 0.4750684 | 0.6865992 | 0.592305 | 0.7827747 |
| Phase-1 RCT-15 | 2.7544286 | 1.888108 | 1.0315828 | 1.7587733 | 1.1863095 | 1.0029835 | 0.9512366 |
| Insulin-like growth factor I | 0.7523039 | 0.4729322 | 0.5585155 | 0.6045762 | 0.6230657 | 0.6181905 | 0.6051048 |
| Sodium/bile acid cotransporter | 1.11165 | 0.4244022 | 0.4948882 | 0.6249808 | 0.5825043 | 0.9208087 | 1.2500373 |
| Organic cation transporter 3 | 0.7917152 | 0.8523673 | 0.7019866 | 0.8357213 | 1.1851923 | 1.0781773 | 1.0039765 |
| Gadd153 | 0.9585432 | 1.3397591 | 1.609979 | 1.2044159 | 1.1566218 | 1.0702122 | 1.0420221 |
| Phase-1 RCT-109 | 1.0222733 | 0.7789074 | 1.0718118 | 0.9086197 | 0.99957 | 0.8401248 | 1.0878533 |
| Phase-1 RCT-88 | 0.7446241 | 0.617419 | 0.6941021 | 1.093092 | 0.969002 | 0.9749044 | 1.0348506 |
| Ubiquitin conjugating enzyme (RAD 6 | 0.6735597 | 0.5113915 | 0.4746227 | 0.6646352 | 1.1945399 | 1.0853708 | 0.883354 |
| homologue) | | | | | | | |
| Glucose-regulated protein 78 | 0.8629929 | 1.3012953 | 1.1101847 | 0.778845 | 1.3610243 | 1.2848794 | 0.9872775 |
| Ribosomal protein L13A | 1.2812889 | 0.7669267 | 1.5516144 | 1.1499544 | 1.0399258 | 0.8083968 | 1.049898 |
| Monocyte chemotactic protein receptor | 1.0943642 | 1.1020247 | 0.8751198 | 0.9763416 | 1.5849905 | 1.3141739 | 0.957688 |
| (CCR2) | | | | | | | |
| Fas antigen | 1.0857185 | 1.6840354 | 1.4177864 | 1.2209109 | 1.1484953 | 1.0751984 | 1.075027 |
| Calpactin I heavy chain | 1.4298669 | 1.2743803 | 1.3019893 | 2.0227895 | 0.9766015 | 1.1365831 | 1.0651437 |
| Uncoupling protein 2 | 0.9638218 | 1.2216827 | 1.3574905 | 1.3630351 | 1.1634705 | 1.0287762 | 1.0612844 |
| Phase-1 RCT-168 | 0.8228663 | 0.660021 | 0.884811 | 0.907633 | 0.9624162 | 0.9264378 | 1.0132192 |
| Phase-1 RCT-154 | 0.9384376 | 1.1625053 | 1.0583947 | 0.9765859 | 1.0426619 | 1.017515 | 0.9638216 |
| Superoxide dismutase Mn | 1.2153718 | 1.2328404 | 1.5455337 | 1.8662498 | 1.1657478 | 1.3223875 | 1.0918903 |
| Phase-1 RCT-214 | 0.9877735 | 0.7023516 | 0.4500406 | 0.731053 | 0.8157693 | 0.7592389 | 0.7714346 |
| Phase-1 RCT-225 | 0.8968247 | 0.7790284 | 0.830626 | 0.8337902 | 0.8679274 | 0.8396468 | 0.8305924 |
| Phase-1 RCT-181 | 0.8478115 | 0.7149683 | 0.7412876 | 1.0285153 | 0.9606195 | 0.9387358 | 1.0020168 |
| Nucleoside diphosphate kinase beta | 0.8403223 | 0.8608511 | 1.0694425 | 0.9453098 | 0.8064604 | 1.0033407 | 0.8130994 |
| isoform | | | | | | | |
| Extracellular-signal-regulated kinase 1 | 1.460816 | 0.9429148 | 0.7128263 | 0.8321872 | 0.633137 | 0.4303031 | 0.9066659 |
| {Ribosomal protein L6} | 1.4821235 | 1.094539 | 0.948311 | 1.1346211 | 0.9895401 | 0.8664298 | 0.945121 |
| Aldehyde dehydrogenase, microsomal | 1.2170534 | 1.0179797 | 0.926531 | 1.0433149 | 0.7985567 | 0.9264132 | 1.0234106 |
| p53 | 1.1559552 | 1.1707462 | 1.0021985 | 1.0253423 | 0.902647 | 0.8291052 | 0.6940784 |
| Phase-1 RCT-239 | 1.2839587 | 1.1768624 | 1.2542163 | 1.0785545 | 1.0845912 | 0.8149808 | 0.8925703 |
| Macrophage inflammatory protein-2 alpha | 1.1103648 | 2.0506077 | 1.9963089 | 2.7863204 | 1.7296944 | 1.3984755 | 1.057463 |
| IgE binding protein | 1.3121263 | 0.9835704 | 0.7496921 | 1.3207936 | 1.0056168 | 1.1103745 | 1.2448051 |
| Phase-1 RCT-205 | 0.8759995 | 1.2586663 | 1.6098799 | 1.3608786 | 0.88207 | 0.9009178 | 0.8740805 |
| Melanoma-associated antigen ME491 | 1.0573484 | 1.0990117 | 1.3235252 | 1.2436352 | 1.1585406 | 1.1992077 | 1.367134 |
| Phase-1 RCT-242 | 1.1013534 | 1.4705359 | 1.6158729 | 1.3519225 | 1.3978821 | 1.4233974 | 0.9008272 |
| Enolase alpha | 0.5726591 | 0.7430877 | 0.667427 | 0.6208412 | 1.1116167 | 1.094056 | 1.2065243 |
| Thymosin beta-10 | 0.7365118 | 0.7543994 | 0.6606314 | 0.8194007 | 1.0188379 | 0.8641261 | 0.8888449 |
| Cofilin | 1.1785523 | 0.6631815 | 0.4511065 | 0.7599531 | 0.8961754 | 0.957858 | 1.044321 |
| Carbonyl reductase | 0.7499322 | 1.188137 | 1.1852311 | 1.012857 | 1.4083107 | 1.2149515 | 0.9839226 |
| MAP kinase kinase | 1.1179197 | 1.1025687 | 1.688682 | 0.9146614 | 0.9365672 | 1.0834298 | 1.0371648 |
| Phase-1 RCT-162 | 0.8184466 | 0.5885994 | 0.5592276 | 0.8091441 | 1.0961819 | 0.9970687 | 1.1164422 |
| Phase-1 RCT-72 | 1.1450472 | 1.5621533 | 2.053513 | 1.3572544 | 2.137234 | 1.0103525 | 0.9559254 |
| Ribosomal protein S8 | 0.7932982 | 0.6102541 | 0.5116074 | 0.8891693 | 1.0724765 | 0.9647878 | 1.2350065 |
| Proliferating cell nuclear antigen gene | 0.7690767 | 1.1448867 | 1.7936352 | 1.0863998 | 1.0963128 | 1.092498 | 1.0831902 |
| Phase-1 RCT-192 | 1.0023295 | 0.9048307 | 0.7248219 | 0.9280626 | 1.0515157 | 1.017537 | 1.1334776 |
| IkB-a | 0.637881 | 0.9117621 | 0.8188443 | 0.8753571 | 1.0033299 | 0.9901904 | 0.9540928 |
| Endogenous retroviral sequence, 5′ and | 1.257166 | 0.9762037 | 1.1741261 | 1.2893927 | 0.7855582 | 0.4760155 | 0.6148866 |
| 3′ LTR | | | | | | | |
| Phase-1 RCT-37 | 1.278767 | 1.3757353 | 1.2542653 | 1.316131 | 1.2276294 | 1.0963138 | 1.0376618 |
| Matrix metalloproteinase-1 | 2.0341387 | 1.9105209 | 1.1983209 | 1.8803958 | 1.2614836 | 1.1598877 | 1.2335101 |
| Cyclin G | 1.5394915 | 1.829506 | 1.8582268 | 1.3973461 | 1.2433742 | 1.0795817 | 0.9329268 |
| DNA polymerase beta | 0.5683401 | 0.4833345 | 0.484386 | 0.5457572 | 0.9486622 | 0.9774564 | 0.894844 |
| Phase-1 RCT-127 | 0.977038 | 1.1935911 | 0.8811882 | 0.8687251 | 1.1119139 | 1.0833609 | 1.0893016 |
| Multidrug resistant protein-2 | 1.4611691 | 1.4736279 | 2.493568 | 1.4167848 | 0.7647978 | 1.1643655 | 1.0687889 |
| Ribosomal protein S9 | 0.5839838 | 0.4780204 | 0.4637142 | 0.6173322 | 1.1296719 | 0.9996365 | 1.0017728 |
| Phase-1 RCT-12 | 1.4226772 | 0.988793 | 1.1604182 | 1.3559804 | 0.9236201 | 0.861105 | 0.7859552 |
| Beta-tubulin, class I | 2.2170424 | 0.9053487 | 1.041209 | 1.5332226 | 0.9906273 | 0.7412758 | 0.6040449 |
| Argininosuccinate lyase | 1.0997481 | 0.5299264 | 0.440427 | 0.7059594 | 1.0781267 | 0.8393408 | 0.9399381 |
| Elongation factor-1 alpha | 0.7846449 | 0.4718441 | 0.6260872 | 0.5756704 | 0.8577111 | 0.8595865 | 1.0858669 |
| Phase-1 RCT-55 | 0.9179155 | 0.8923527 | 1.2911153 | 1.0229619 | 0.8150675 | 0.8658056 | 1.0613422 |
| Macrophage inflammatory protein-1 alpha | 1.0065124 | 1.7612402 | 1.448358 | 1.4732945 | 1.5228513 | 1.9438553 | 1.0590323 |
| c-H-ras | 0.9970922 | 1.075605 | 0.9131969 | 1.1045375 | 1.1841125 | 1.0201089 | 0.8969856 |
| Multidrug resistant protein-1 | 1.457863 | 2.064454 | 2.0380626 | 1.5686344 | 0.8855707 | 1.2932029 | 1.0923005 |
| T-cell cyclophilin | 0.5227771 | 0.3670058 | 0.3732305 | 0.4511427 | 0.846504 | 0.9741869 | 0.9634313 |
| Phase-1 RCT-185 | 0.7734824 | 0.5531105 | 0.443651 | 0.6615576 | 0.7162942 | 0.6009344 | 0.7856274 |
| 25-DX | 0.8788996 | 0.6988992 | 0.8532984 | 0.7774722 | 0.719973 | 0.7917166 | 0.8876879 |
| Ref-1 | 1.0843921 | 0.9431994 | 1.0215381 | 1.2592876 | 0.8494004 | 1.1100491 | 0.9465116 |
| Apolipoprotein CIII | 0.8716801 | 1.1424757 | 1.0567625 | 1.0036926 | 0.8977446 | 0.7266086 | 0.8962024 |
| NADPH quinone oxidoreductase-1 (DT- | 1.0180007 | 1.3543009 | 1.0937251 | 1.6516302 | 0.8814444 | 1.8304617 | 0.9703786 |
| diaphorase) | | | | | | | |
| Pyruvate kinase, muscle | 1.0657641 | 0.9227019 | 0.9424861 | 0.9132966 | 1.8802722 | 1.0500202 | 0.9304692 |
| | PUR | PUR | PUR | PUR | PUR | PUR | QUIN |
| Compound-Dose (2) | 38 | 38 | 38 | 150 | 150 | 150 | 25 |
| Animal Number (3) | 24 | 25 | 26 | 34 | 35 | 36 | 2544 |
| Liver Toxicity Necrosis Classification (4) | no | no | no | no | no | no | no |
| Gene Name (5) | | | | | | | |
| Phase-1 RCT-78 | 1.0645751 | 1.3102221 | 1.0358194 | 0.9833112 | 1.178431 | 1.2139636 | 1.0527768 |
| Gamma-actin, cytoplasmic | 1.2848973 | 1.1303768 | 0.8741316 | 0.8973408 | 0.9633074 | 0.9028386 | 0.612314 |
| Matrin F/G | 1.0897698 | 1.1369069 | 1.1833751 | 1.0919164 | 1.3273002 | 1.1794827 | 0.965327 |
| Zinc finger protein | 0.7768126 | 0.9931212 | 1.1407169 | 0.9537212 | 0.8500998 | 0.9338582 | 0.9741769 |
| Cathepsin L, sequence 2 | 1.1170532 | 0.8731833 | 0.961345 | 0.6753056 | 1.6066571 | 1.4191232 | 0.8652589 |
| Gadd45 | 1.2352792 | 0.8369449 | 1.0425459 | 0.713066 | 1.0580709 | 1.250975 | 1.0638925 |
| Phase-1 RCT-144 | 0.9456663 | 1.109673 | 1.1624205 | 1.0807217 | 1 | 1.0626467 | 1.0332111 |
| Phase-1 RCT-145 | 0.8144794 | 0.8864563 | 1.0455493 | 0.9726354 | 0.887562 | 0.8683834 | 0.9849822 |
| Phase-1 RCT-50 | 0.9661176 | 0.961558 | 0.9650111 | 0.901342 | 1.0295298 | 1.0279858 | 1.1204293 |
| Phase-1 RCT-92 | 0.742674 | 0.8431694 | 0.9916127 | 1.1340371 | 0.7979187 | 0.7569069 | 0.9886882 |
| Phase-1 RCT-89 | 1.0716478 | 0.9623397 | 1.1261408 | 1.0327787 | 0.9030214 | 0.8904563 | 0.914287 |
| 14-3-3 zeta | 0.9568889 | 0.9590102 | 0.9520241 | 1.1135389 | 1.3315772 | 1.1425169 | 1.2764939 |
| Dynamin-1 (D100) | 1.2631251 | 1.4042493 | 1.4516655 | 1.4037815 | 1.1441544 | 1.2282858 | 0.9602488 |
| Insulin-like growth factor binding protein 1 | 0.9941923 | 0.8614938 | 0.7224566 | 0.8854182 | 0.9580864 | 0.9260048 | 1.0910687 |
| L-gulono-gamma-lactone oxidase | 1.1302024 | 1.3204004 | 1.147117 | 1.3021693 | 1.3143433 | 0.9139708 | 0.9282004 |
| Ornithine decarboxylase | 1.130574 | 0.7855509 | 0.9002153 | 1.0718437 | 1.2900862 | 1.246349 | 1.0990025 |
| PAR interacting protein | 0.8988333 | 1.022275 | 1.0279727 | 1.0413941 | 0.9575956 | 0.9450212 | 0.981501 |
| Phase-1 RCT-128 | 1.3482887 | 1.4294463 | 1.5774268 | 1.3951895 | 1.1270227 | 1.1694187 | 0.973375 |
| Phase-1 RCT-180 | 0.8562512 | 1.0432384 | 1.1505985 | 1.0967757 | 0.9647813 | 0.9771681 | 0.9022963 |
| Phase-1 RCT-182 | 1.0259411 | 1.1687399 | 1.0248908 | 1.1517327 | 1.0442426 | 0.9553665 | 0.9309076 |
| Phase-1 RCT-207 | 1.0481902 | 1.1356007 | 1.0403031 | 1.0496986 | 1.2099297 | 1.1358478 | 1.0533913 |
| Phase-1 RCT-213 | 0.9593037 | 1.0469502 | 0.9790719 | 0.9415407 | 1.3756757 | 1.23126 | 0.9730299 |
| Phase-1 RCT-256 | 0.9705028 | 0.9894408 | 1.2411373 | 1.1835926 | 0.9432303 | 0.7713182 | 0.9835368 |
| Phase-1 RCT-258 | 0.9697112 | 1.012419 | 1.0557699 | 0.9929546 | 1.2268956 | 1.0505431 | 1.0234112 |
| Phase-1 RCT-264 | 1.2500962 | 1.2125511 | 1.2161921 | 0.7314287 | 1.1579235 | 1.0496624 | 0.6563345 |
| Phase-1 RCT-271 | 1.3053808 | 1.0597711 | 1.2246428 | 1.3002732 | 1.4238212 | 1.2854012 | 1.0111132 |
| Phase-1 RCT-288 | 0.8319045 | 0.9258976 | 0.9117199 | 0.7768823 | 0.9277865 | 0.7552323 | 0.9853258 |
| Phase-1 RCT-38 | 1.1942757 | 1.1176802 | 1.3505039 | 1.4675643 | 1.1601557 | 1.1753129 | 0.9473301 |
| Phase-1 RCT-39 | 1.2273827 | 1.0080364 | 0.9785401 | 0.997368 | 1.3958341 | 1.6314013 | 0.9829932 |
| Phase-1 RCT-68 | 1.2920259 | 1.1587706 | 1.1729709 | 1.0420145 | 1.5095077 | 1.517062 | 1.0908796 |
| Phase-1 RCT-33 | 0.8334774 | 0.9216842 | 0.9133976 | 1.1606656 | 0.9348851 | 0.9159316 | 0.9808205 |
| Phase-1 RCT-36 | 0.822591 | 0.8774204 | 0.8906821 | 1.1700587 | 0.9178675 | 0.9002619 | 1.0077785 |
| 17-beta hydroxysteroid dehydrogenase, | 0.5608776 | 0.4379293 | 0.6398925 | 0.3709935 | 0.3478741 | 0.4546317 | 1.002293 |
| type 2 | | | | | | | |
| Senescence marker protein-30 | 1.2950164 | 1.4567496 | 1.1480064 | 0.7115146 | 1.4083627 | 1.0917314 | 0.8071643 |
| Ribosomal protein S17 | 1.3178543 | 1.1187488 | 1.3357558 | 1.0542296 | 1.5789634 | 1.3416605 | 0.8053106 |
| Phase-1 RCT-83 | 1.095141 | 1.016966 | 1.0750015 | 0.8029293 | 0.8589164 | 0.9059128 | 0.8786108 |
| Phase-1 RCT-49 | 0.8361505 | 0.9574115 | 1.0312035 | 1.0262363 | 1.0053183 | 0.9464631 | 0.9891065 |
| Phase-1 RCT-48 | 1.3321055 | 1.3306913 | 1.39749 | 1.3580681 | 1.3607554 | 1.6720998 | 0.867276 |
| Phase-1 RCT-40 | 0.983988 | 0.9155852 | 1.0219839 | 0.9395569 | 0.8790186 | 0.8177124 | 0.8181292 |
| Phase-1 RCT-296 | 1.171437 | 1.2054237 | 1.3876404 | 1.1029518 | 1.1496809 | 1.0546476 | 0.8720876 |
| Phase-1 RCT-295 | 1.4344728 | 1.3778384 | 1.3530191 | 1.086432 | 2.0536628 | 1.9678106 | 0.9171631 |
| Phase-1 RCT-291 | 1.031639 | 1.0547712 | 1.0157382 | 1.0039705 | 0.9838791 | 1.0691692 | 0.9678504 |
| Phase-1 RCT-270 | 1.0262761 | 1.1621456 | 1.0632836 | 0.9129586 | 0.9972496 | 0.8483733 | 0.7375349 |
| Phase-1 RCT-241 | 1.0124931 | 0.9630087 | 1.0166425 | 1.0185623 | 0.9574187 | 1.0172418 | 1.0735934 |
| Phase-1 RCT-191 | 0.8291966 | 0.9879354 | 1.0472698 | 1.0497805 | 0.9038601 | 1.0679215 | 1.0490595 |
| Phase-1 RCT-189 | 0.6622378 | 0.7704001 | 0.7518947 | 0.9512751 | 0.605778 | 0.6499617 | 1.1656815 |
| Phase-1 RCT-179 | 0.9817542 | 0.9861224 | 1.0304334 | 0.8951941 | 1.0537909 | 0.9019953 | 0.8679042 |
| Phase-1 RCT-152 | 1.4358506 | 1.2055336 | 1.3466654 | 1.1473169 | 1.4201219 | 1.3616962 | 0.7803856 |
| Phase-1 RCT-123 | 1.048335 | 1.1027435 | 1.0391377 | 1.174786 | 1.3259852 | 1.1791644 | 1.0492045 |
| Paraoxonase 1 | 0.9374228 | 0.8773083 | 0.9030238 | 0.7476889 | 0.965768 | 0.8308595 | 0.7005753 |
| Organic anion transporter 3 | 1.3766134 | 1.1520414 | 0.9718759 | 0.6753391 | 0.9940133 | 1.295898 | 1.0355397 |
| N-hydroxy-2-acetylaminofluorene | 1.1105255 | 1.0789756 | 1.1346847 | 0.8181921 | 0.9558696 | 0.9955902 | 0.7756133 |
| sulfotransferase (ST1C1) | | | | | | | |
| Integrin beta1 | 1.2588307 | 1.0338233 | 1.0156817 | 1.001623 | 1.1787779 | 1.2420021 | 1.0504164 |
| Insulin-like growth factor binding protein 3 | 0.4728847 | 0.7265811 | 0.6864435 | 0.7031336 | 0.5576699 | 0.4675067 | 0.7952873 |
| ID-1 | 1.0344301 | 1.0343043 | 1.0095199 | 1.1160401 | 1.2304094 | 1.5455824 | 1.0428363 |
| Hepatic lipase | 0.7212262 | 0.8440919 | 0.908092 | 1.2869017 | 0.7358881 | 0.9080542 | 1.0174009 |
| Heme oxygenase | 0.9008815 | 0.7268839 | 0.8279591 | 0.6583206 | 0.5392975 | 0.6662464 | 0.9022995 |
| Equilbrative nitrobenzylthioinosine- | 1.1053175 | 0.9831296 | 0.8751978 | 0.8580609 | 0.9205711 | 0.7264434 | 0.9230696 |
| sensitive nucleoside transporter | | | | | | | |
| Epidermal growth factor | 1.1656106 | 1.0558574 | 0.9636077 | 0.9935051 | 1.0826428 | 0.9197633 | 0.9753654 |
| c-myc | 1.6251683 | 1.1507179 | 1.3247337 | 1.2174094 | 1.0964655 | 1.1176052 | 1.0682324 |
| Carbonic anhydrase III | 1.3313161 | 1.7878973 | 1.2322781 | 0.9279815 | 1.5931683 | 1.2893487 | 0.4595247 |
| Beta-actin | 0.5631421 | 0.7085763 | 0.6266839 | 0.8900166 | 0.9898194 | 1.0581359 | 0.9380752 |
| Bax (alpha) | 1.4588511 | 1.137699 | 1.1792024 | 1.0228235 | 2.097134 | 2.4598 | 1.0816919 |
| Alpha 1 - inhibitor III | 0.5010947 | 0.7272087 | 0.6650058 | 0.8955242 | 0.4601556 | 0.5236825 | 0.7907036 |
| 60S ribosomal protein L6 | 1.2056105 | 1.000431 | 1.0448604 | 0.9357622 | 1.3056123 | 1.1687535 | 0.8685022 |
| Phase-1 RCT-117 | 0.8684437 | 1.2388357 | 0.9838044 | 1.242084 | 1.43395 | 1.1158992 | 1.4738036 |
| Phase-1 RCT-102 | 0.759895 | 0.8703557 | 1.0030366 | 0.8884 | 0.7798803 | 0.8260614 | 0.8449023 |
| 3-hydroxyisobutyrate dehydrogenase | 0.9191102 | 0.7812628 | 0.8474102 | 0.7352387 | 0.7804788 | 0.768648 | 0.9527898 |
| ATPase inhibitor (rat mitochondrial IF1 | 0.9911843 | 0.897929 | 1.0071806 | 0.7950053 | 0.9727635 | 0.7860936 | 0.75896 |
| protein) | | | | | | | |
| Alpha-2-macroglobulin | 1.3099949 | 1.032604 | 1.0448245 | 0.9047291 | 0.9964047 | 1.1658968 | 0.9921243 |
| Phase-1 RCT-137 | 1.1105427 | 1.2337664 | 1.2455503 | 1.1979705 | 1.0129396 | 0.8419915 | 0.8895667 |
| Phase-1 RCT 252 | 0.6771212 | 0.7271106 | 0.7009793 | 1.1950767 | 0.7466064 | 0.6892731 | 0.9081184 |
| Phase-1 RCT-65 | 0.9783447 | 1.0579706 | 0.9367471 | 1.0149153 | 1.1736362 | 1.1776758 | 0.9765218 |
| Liver fatty acid binding protein | 1.0385555 | 0.9338476 | 0.8142077 | 0.7219544 | 0.6314094 | 0.4811154 | 0.6926771 |
| Carbamyl phosphate synthetase I | 0.5410494 | 0.6529418 | 0.4993969 | 0.9985298 | 0.3419062 | 0.4045962 | 1.2719905 |
| c-jun | 1.1534694 | 0.9099327 | 0.8782947 | 0.9707161 | 0.9287175 | 1.2017705 | 1.495081 |
| Transthyretin | 1.3479048 | 1.288237 | 1.1993573 | 0.5830997 | 1.3110855 | 1.302487 | 0.5777658 |
| Aflatoxin B1 aldehyde reductase | 1.7730619 | 1.6093724 | 1.6672593 | 1.158128 | 2.226149 | 1.5160763 | 0.8632206 |
| Phase-1 RCT-15 | 0.9143987 | 0.833034 | 1.000939 | 0.8048183 | 0.8277987 | 0.8890795 | 0.9629573 |
| Insulin-like growth factor I | 0.4933031 | 0.7512466 | 0.6624364 | 0.7470843 | 0.5205467 | 0.4487665 | 0.8053352 |
| Sodium/bile acid cotransporter | 1.1937184 | 1.2177778 | 0.9546348 | 0.8489714 | 0.4453941 | 0.4302045 | 0.8406497 |
| Organic cation transporter 3 | 1.0804435 | 0.999319 | 1.018497 | 0.9730313 | 1.3232245 | 1.1803985 | 0.9002472 |
| Gadd153 | 1.2556634 | 1.1195099 | 0.9997616 | 1.0199667 | 1.4937395 | 1.6063533 | 1.0571584 |
| Phase-1 RCT-109 | 1.336296 | 1.0619586 | 1.1016163 | 1.1838129 | 1.2741897 | 1.2177144 | 0.9770686 |
| Phase-1 RCT-88 | 0.630362 | 0.836838 | 0.8634356 | 0.9481176 | 0.7322763 | 0.7023335 | 1.1543163 |
| Ubiquitin conjugating enzyme (RAD 6 | 1.3020006 | 1.0105556 | 1.0456108 | 1.0039783 | 1.1488535 | 1.0668669 | 1 |
| homologue) | | | | | | | |
| Glucose-regulated protein 78 | 1.0876074 | 0.8751145 | 1.12076 | 0.9214653 | 0.721667 | 0.8717648 | 1.1143619 |
| Ribosomal protein L13A | 1.2094421 | 0.9584273 | 0.9738517 | 1.0541776 | 1.2533122 | 1.2657664 | 1.0345268 |
| Monocyte chemotactic protein receptor | 1.1704521 | 1.0452675 | 1.041433 | 0.9246817 | 1.3397326 | 1.1860888 | 0.9538322 |
| (CCR2) | | | | | | | |
| Fas antigen | 0.6996766 | 0.7643318 | 0.8229191 | 0.8949409 | 0.9425489 | 0.9752446 | 1.1569496 |
| Calpactin I heavy chain | 0.9917087 | 1.0060629 | 1.0571504 | 1.1765889 | 0.9755399 | 0.9074196 | 1.3389153 |
| Uncoupling protein 2 | 1.18393 | 1.0291772 | 0.9705129 | 1.0171777 | 1.2171835 | 1.2649333 | 0.9988946 |
| Phase-1 RCT-168 | 0.6931724 | 0.8581658 | 0.8898843 | 0.9698585 | 0.8206906 | 0.7907286 | 1.0064405 |
| Phase-1 RCT-154 | 0.8658128 | 0.9149554 | 1.0516373 | 0.9491661 | 1.2580551 | 1.1580461 | 1.0026473 |
| Superoxide dismutase Mn | 1.2262337 | 1.1622386 | 1.0997316 | 1.253161 | 1.4829301 | 1.5276966 | 1.1517783 |
| Phase-1 RCT-214 | 0.7349864 | 0.8793865 | 1.0243044 | 0.7318093 | 0.7890015 | 0.8145323 | 0.9518482 |
| Phase-1 RCT-225 | 1.1244746 | 1.135909 | 1.0773937 | 1.2216027 | 1.0403862 | 0.9120135 | 0.9274929 |
| Phase-1 RCT-181 | 0.7300708 | 0.8744831 | 0.8898559 | 1.1257982 | 0.876453 | 0.7705918 | 1.0240157 |
| Nucleoside diphosphate kinase beta | 1.1428217 | 1.0686893 | 1.2417622 | 1.3093783 | 1.6616473 | 1.661652 | 1.1352782 |
| isoform | | | | | | | |
| Extracellular-signal-regulated kinase 1 | 1.3258423 | 1.7916913 | 1.2455004 | 0.8739165 | 1.5924586 | 1.2994815 | 0.4759278 |
| {Ribosomal protein L6} | 1.0044655 | 1.0035588 | 1.1111387 | 1.1560725 | 1.2057074 | 1.0045232 | 0.9113089 |
| Aldehyde dehydrogenase, microsomal | 1.0275314 | 1.0598164 | 0.9768148 | 0.8841077 | 1.1067256 | 1.0000972 | 1.0611748 |
| p53 | 1.357921 | 1.222491 | 1.3202943 | 0.9914671 | 1.5239137 | 1.3973541 | 0.990631 |
| Phase-1 RCT-239 | 0.9168713 | 0.9562604 | 0.9769846 | 0.9842565 | 0.9389484 | 0.8786098 | 1.0072464 |
| Macrophage inflammatory protein-2 alpha | 1.225336 | 0.9602098 | 0.8469707 | 0.9782754 | 1.0913934 | 1.2490882 | 1.0232733 |
| IgE binding protein | 0.9465332 | 0.9184237 | 0.9396217 | 1.1446973 | 0.8509752 | 0.8100493 | 0.9262412 |
| Phase-1 RCT-205 | 0.8999713 | 1.1021888 | 1.1120857 | 1.1570783 | 1.0365773 | 1.0104114 | 1.0370882 |
| Melanoma-associated antigen ME491 | 0.8596842 | 1.0414617 | 1.073381 | 1.1359566 | 0.9044821 | 0.9142397 | 0.8258411 |
| Phase-1 RCT-242 | 0.9845648 | 0.9700783 | 1.0476485 | 1.0485517 | 0.9553069 | 0.8712794 | 0.9218593 |
| Enolase alpha | 0.726719 | 0.7704854 | 1.2043334 | 1.0908909 | 0.9570065 | 0.954432 | 0.8879379 |
| Thymosin beta-10 | 1.140737 | 1.0670395 | 1.0025921 | 1.0346295 | 1.192718 | 1.4170622 | 1.0796021 |
| Cofilin | 0.826383 | 0.7711769 | 0.9933131 | 0.8558019 | 0.7358745 | 0.8059508 | 0.9171013 |
| Carbonyl reductase | 1.3604774 | 1.1263167 | 1.0562131 | 1.1265755 | 1.3611407 | 1.4276092 | 0.9945502 |
| MAP kinase kinase | 1.0612943 | 0.9516887 | 0.8983493 | 0.7943596 | 1.0270408 | 0.9867942 | 1.0379138 |
| Phase-1 RCT-162 | 1.1670817 | 1.0663964 | 1.1210763 | 0.9660404 | 1.5449513 | 1.2036796 | 0.8324402 |
| Phase-1 RCT-72 | 1.1484046 | 1.087422 | 0.9856677 | 0.9919738 | 0.9819115 | 1.0522423 | 0.9345928 |
| Ribosomal protein S8 | 1.1453567 | 0.9190009 | 1.0683914 | 0.8086751 | 1.5305461 | 1.0900308 | 0.7938675 |
| Proliferating cell nuclear antigen gene | 1.2701594 | 1.1024979 | 1.0905153 | 1.1835924 | 1.2587943 | 1.2422686 | 1.0032842 |
| Phase-1 RCT-192 | 0.8910161 | 0.950271 | 0.8360848 | 0.8933413 | 0.8471299 | 0.8022142 | 0.894142 |
| IkB-a | 0.8096273 | 0.8581718 | 1.184879 | 1.1036737 | 0.9815763 | 1.1270441 | 0.982654 |
| Endogenous retroviral sequence, 5′ and | 0.8245942 | 0.9798462 | 0.796095 | 1.1136726 | 0.6842027 | 0.7589394 | 1.2964071 |
| 3′ LTR | | | | | | | |
| Phase-1 RCT-37 | 1.0958736 | 1.0115653 | 1.1459687 | 1.1291045 | 1.3615165 | 1.1674701 | 0.9152616 |
| Matrix metalloproteinase-1 | 0.9648064 | 0.9008418 | 0.8584962 | 0.9247216 | 1.2480894 | 1.0420702 | 0.8634478 |
| Cyclin G | 1.3733817 | 1.2240802 | 1.2335131 | 1.0193083 | 3.0348942 | 3.27404 | 1.0690874 |
| DNA polymerase beta | 1.4488252 | 1.1775945 | 1.2574115 | 1.0293976 | 1.6505729 | 1.4318742 | 0.7639067 |
| Phase-1 RCT-127 | 0.9145214 | 1.1795388 | 0.9753509 | 1.2020861 | 1.1940215 | 1.1048177 | 1.2800359 |
| Multidrug resistant protein-2 | 1.0602669 | 0.8333289 | 0.6475833 | 0.6938478 | 1.2262144 | 1.3631281 | 0.9802222 |
| Ribosomal protein S9 | 1.2472284 | 1.1852968 | 1.1293916 | 1.0733988 | 1.1459048 | 0.9726019 | 1.0030357 |
| Phase-1 RCT-12 | 1.1512918 | 1.088557 | 1.0909538 | 1.3421401 | 1.1065127 | 1.0722913 | 0.9930476 |
| Beta-tubulin, class I | 1.100314 | 1.0975437 | 1.0962585 | 1.3462931 | 1.2587037 | 1.0853671 | 0.9188387 |
| Argininosuccinate lyase | 1.1377493 | 1.1892755 | 1.1367239 | 0.7499071 | 1.5288361 | 1.339789 | 0.9366055 |
| Elongation factor-1 alpha | 0.7748804 | 0.874375 | 0.8208095 | 0.8294946 | 1.2255888 | 0.9836854 | 0.8522121 |
| Phase-1 RCT-55 | 1.0139458 | 0.9878404 | 0.8154912 | 0.9137604 | 0.9101641 | 0.8883212 | 0.6885313 |
| Macrophage inflammatory protein-1 alpha | 1.1490759 | 1.0857837 | 1.0236334 | 1.1692392 | 0.975223 | 1.1822815 | 1.0687159 |
| c-H-ras | 0.7691484 | 0.819843 | 0.8678842 | 0.9555515 | 0.9057145 | 0.8208283 | 1.1245339 |
| Multidrug resistant protein-1 | 0.9124842 | 0.7349091 | 0.6637427 | 0.7723559 | 1.2728543 | 1.3562922 | 1.1525831 |
| T-cell cyclophilin | 1.5402752 | 1.1922938 | 1.3443205 | 1.0303429 | 1.7743069 | 1.4598501 | 0.7524583 |
| Phase-1 RCT-185 | 1.2947125 | 1.3388631 | 1.251327 | 1.2782363 | 1.197667 | 1.1729552 | 0.8063707 |
| 25-DX | 1.1569953 | 1.2715315 | 1.0687116 | 1.0707048 | 1.0833657 | 0.8991572 | 1.0036285 |
| Ref-1 | 1.380583 | 1.1685477 | 1.0947565 | 0.7263802 | 0.8584321 | 1.197389 | 0.8836225 |
| Apolipoprotein CIII | 0.9834256 | 0.9314295 | 0.8833951 | 0.7649999 | 0.7922566 | 0.7512313 | 1.1150836 |
| NADPH quinone oxidoreductase-1 (DT- | 1.4107747 | 1.1488516 | 1.0367094 | 0.6270587 | 0.7860948 | 1.1644568 | 0.9057592 |
| diaphorase) | | | | | | | |
| Pyruvate kinase, muscle | 1.0732787 | 0.8730691 | 0.9927813 | 0.7240837 | 1.8328377 | 1.5357057 | 0.9472378 |
| | QUIN | QUIN | QUIN | QUIN | QUIN | STRZ | STRZ |
| Compound-Dose (2) | 25 | 25 | 100 | 100 | 100 | 20 | 20 |
| Animal Number (3) | 2545 | 2546 | 2554 | 2555 | 2556 | 1724 | 1725 |
| Liver Toxicity Necrosis Classification (4) | no | no | no | no | no | no | no |
| Gene Name (5) | | | | | | | |
| Phase-1 RCT-78 | 1.1466976 | 0.884071 | 1.1764361 | 1.0111942 | 1.340157 | 1.1784865 | 1.1333748 |
| Gamma-actin, cytoplasmic | 0.7852165 | 0.8106768 | 0.9609373 | 1.1256561 | 0.6757434 | 1.3477448 | 1.0740211 |
| Matrin F/G | 0.90608 | 1.0432537 | 1.0802516 | 1.0441173 | 1.1225145 | 1.5043013 | 1.4361528 |
| Zinc finger protein | 1.0538012 | 1.0487958 | 0.923312 | 0.9406965 | 1.0509906 | 0.9148063 | 0.9364402 |
| Cathepsin L, sequence 2 | 0.9490078 | 0.7983698 | 0.8187811 | 0.9875888 | 0.972319 | 1.1460361 | 0.9688725 |
| Gadd45 | 1.0285738 | 1.2553918 | 1.0119573 | 0.9645712 | 1.1345664 | 1.0406842 | 0.8937236 |
| Phase-1 RCT-144 | 0.9639077 | 1.0333773 | 0.9824363 | 0.9722816 | 0.9856836 | 0.8702669 | 0.9266033 |
| Phase-1 RCT-145 | 1.007056 | 1.1272136 | 0.9030812 | 0.9460653 | 0.9486363 | 0.8247132 | 0.9357027 |
| Phase-1 RCT-50 | 0.9973784 | 1.0509244 | 1.0178111 | 1.1140054 | 1.0114877 | 1.1989232 | 1.01556 |
| Phase-1 RCT-92 | 1.050027 | 1.0700197 | 0.9051601 | 0.7789363 | 0.8632326 | 0.7739993 | 0.9422649 |
| Phase-1 RCT-89 | 1.0229722 | 0.9053153 | 0.8845675 | 0.877543 | 0.9362536 | 1.0719113 | 1.1987218 |
| 14-3-3 zeta | 1.0721986 | 1.2248434 | 1.0150906 | 0.9746005 | 1.0861968 | 1.2149022 | 1.2562095 |
| Dynamin-1 (D100) | 1.0046074 | 1.0061039 | 0.9741057 | 0.9410347 | 0.927426 | 0.8303059 | 0.9423155 |
| Insulin-like growth factor binding protein 1 | 0.8610951 | 1.1802 | 0.9958953 | 1.2431123 | 1.1284914 | 1.1971848 | 1.2669328 |
| L-gulono-gamma-lactone oxidase | 1.0204476 | 0.885394 | 0.7354151 | 0.6239225 | 0.8898928 | 0.9267995 | 1.2334241 |
| Ornithine decarboxylase | 0.8511241 | 1.0366936 | 1.097515 | 1.0889164 | 1.1012329 | 1.2317274 | 1.311825 |
| PAR interacting protein | 0.886677 | 0.9896208 | 0.9378233 | 1.0292124 | 0.9942618 | 0.9194245 | 1.0559256 |
| Phase-1 RCT-128 | 0.9817954 | 0.9459377 | 0.8776458 | 0.7291102 | 0.833624 | 0.787941 | 0.9451293 |
| Phase-1 RCT-180 | 0.9802226 | 1.1095995 | 0.8750562 | 1.2420223 | 1.0840552 | 1.2287831 | 1.2865108 |
| Phase-1 RCT-182 | 1.143107 | 0.8591301 | 0.8404985 | 0.9309737 | 0.8047298 | 1.23799 | 0.9365764 |
| Phase-1 RCT-207 | 1.0457318 | 1.0008882 | 0.9847255 | 0.9757971 | 1.0530064 | 1.0709745 | 1.0714896 |
| Phase-1 RCT-213 | 0.9930256 | 0.9969445 | 0.9820188 | 1.0823132 | 1.0785264 | 0.9648402 | 1.1185992 |
| Phase-1 RCT-256 | 0.8769985 | 0.9074374 | 0.9002881 | 0.8131173 | 0.8930183 | 1.2603745 | 1.1243145 |
| Phase-1 RCT-258 | 1.0386927 | 0.9617716 | 0.964348 | 0.9888415 | 0.9675319 | 0.8763658 | 1.0726936 |
| Phase-1 RCT-264 | 0.8722705 | 0.9927683 | 1.0229362 | 0.6171362 | 0.6302084 | 1.1182985 | 1.0955241 |
| Phase-1 RCT-271 | 0.9738002 | 0.8515387 | 0.8913112 | 0.5996124 | 0.7158492 | 0.8445312 | 0.9606769 |
| Phase-1 RCT-288 | 1.0353199 | 0.8881396 | 0.920632 | 0.7706345 | 0.9369745 | 0.7179508 | 0.8025644 |
| Phase-1 RCT-38 | 0.8184298 | 0.8336515 | 0.9204643 | 0.7506074 | 0.8808454 | 1.1516895 | 1.0045294 |
| Phase-1 RCT-39 | 0.9646224 | 0.9479268 | 1.0064684 | 1.0041528 | 1.0701339 | 1.216671 | 0.9455292 |
| Phase-1 RCT-68 | 1.069446 | 1.160955 | 1.0912114 | 1.0918872 | 1.1003314 | 1.2893646 | 1.2135209 |
| Phase-1 RCT-33 | 0.9758685 | 1.1426284 | 1.1060886 | 0.9611391 | 0.9256947 | 0.8179846 | 1.3986614 |
| Phase-1 RCT-36 | 0.9509437 | 1.1752667 | 1.0313971 | 0.9426526 | 0.9334667 | 0.8567656 | 1.2365443 |
| 17-beta hydroxysteroid dehydrogenase, | 0.6711866 | 2.297562 | 0.906037 | 0.8008974 | 1.0133876 | 0.7514007 | 0.9782514 |
| type 2 | | | | | | | |
| Senescence marker protein-30 | 0.9429829 | 0.742879 | 0.6931003 | 0.6638716 | 0.9256622 | 1.0774349 | 0.9654012 |
| Ribosomal protein S17 | 0.8427842 | 0.7282682 | 0.8738632 | 0.9343398 | 0.8902003 | 1.4447832 | 1.0014515 |
| Phase-1 RCT-83 | 0.997022 | 0.8433844 | 1.1215061 | 0.9146977 | 0.9927731 | 1.2680068 | 0.9343785 |
| Phase-1 RCT-49 | 0.9762161 | 1.0469346 | 0.9781011 | 0.9308864 | 0.929161 | 0.8051223 | 0.8725287 |
| Phase-1 RCT-48 | 1.093708 | 1.1354841 | 0.9714905 | 1.0817555 | 0.9418899 | 1.0141337 | 1.3387886 |
| Phase-1 RCT-40 | 1.0292321 | 0.9393413 | 0.9533367 | 0.8775898 | 0.999602 | 1.0271748 | 1.1479387 |
| Phase-1 RCT-296 | 0.9217466 | 1.2038747 | 0.9747702 | 0.752346 | 0.8411648 | 0.7363155 | 1.1081543 |
| Phase-1 RCT-295 | 0.9677233 | 0.9878281 | 1.0370538 | 1.0110915 | 1.0163696 | 1.4791473 | 1.2455173 |
| Phase-1 RCT-291 | 1.0306625 | 1.1111158 | 0.9558812 | 0.9699153 | 0.925817 | 1.1226672 | 1.0325527 |
| Phase-1 RCT-270 | 0.8722975 | 0.8671903 | 0.9313459 | 0.7765517 | 0.7747057 | 1.1562512 | 1.1493598 |
| Phase-1 RCT-241 | 1.0146104 | 1.0772859 | 1.000369 | 1.3326259 | 1.1794724 | 0.7512681 | 0.9577642 |
| Phase-1 RCT-191 | 1.0046977 | 1.0667682 | 1.0659876 | 1.2278448 | 1.1364936 | 1.3972138 | 1.4683541 |
| Phase-1 RCT-189 | 1.1115472 | 1.121845 | 1.1046964 | 1.1420656 | 1.0894144 | 0.7995531 | 0.9014626 |
| Phase-1 RCT-179 | 0.9538171 | 1 | 0.9465466 | 0.7886449 | 0.9300162 | 0.8453957 | 1.1375991 |
| Phase-1 RCT-152 | 0.8907142 | 0.8566635 | 0.8827247 | 0.9354856 | 0.8861281 | 0.9665398 | 0.8927398 |
| Phase-1 RCT-123 | 1.0025271 | 0.7849307 | 0.956364 | 1.068137 | 1.1618915 | 1.0668241 | 0.9700902 |
| Paraoxonase 1 | 0.899886 | 0.7902314 | 0.8413775 | 0.6228406 | 0.7733092 | 0.7860919 | 0.7640654 |
| Organic anion transporter 3 | 0.9739757 | 0.9001047 | 0.872512 | 0.6483651 | 0.8844133 | 1.2361566 | 1.026043 |
| N-hydroxy-2-acetylaminofluorene | 1.0369519 | 0.8992589 | 0.9243876 | 0.6942487 | 0.9648516 | 0.8947344 | 0.7487291 |
| sulfotransferase (ST1C1) | | | | | | | |
| Integrin beta1 | 0.9497713 | 1.1477582 | 1.0597669 | 1.319445 | 1.165864 | 1.0735962 | 1.0158949 |
| Insulin-like growth factor binding protein 3 | 0.8320833 | 1.1398792 | 1.0279968 | 0.9303042 | 1.0243487 | 1.2578481 | 1.2672284 |
| ID-1 | 1.0884262 | 1.1371402 | 0.9864688 | 0.8374087 | 0.863856 | 1.2748535 | 1.3296186 |
| Hepatic lipase | 0.7683316 | 0.9481161 | 0.8246586 | 0.706828 | 0.7741934 | 0.559352 | 0.6630793 |
| Heme oxygenase | 1.0019865 | 0.9881182 | 0.9866383 | 0.9235224 | 0.8770436 | 1.2609836 | 1.0491647 |
| Equilbrative nitrobenzylthioinosine- | 1.002388 | 0.8216993 | 0.9610089 | 0.8252681 | 0.9625236 | 1.0478361 | 0.8377422 |
| sensitive nucleoside transporter | | | | | | | |
| Epidermal growth factor | 1.0146859 | 1.0288634 | 1.0349494 | 1.0644085 | 1.033515 | 0.8729655 | 0.7990432 |
| c-myc | 0.9556943 | 1.38997 | 0.964079 | 1.0781676 | 1.0153041 | 0.9989564 | 1.0307853 |
| Carbonic anhydrase III | 1.0082513 | 0.7632411 | 0.5389375 | 0.3010709 | 0.6195228 | 1.3401947 | 1.0709704 |
| Beta-actin | 0.9972961 | 1.0483242 | 0.9683616 | 1.1216648 | 0.9036032 | 1.4153093 | 1.4022264 |
| Bax (alpha) | 0.9440556 | 1.2334515 | 1.1071482 | 1.2510041 | 1.1127865 | 1.4675487 | 1.5156664 |
| Alpha 1 - inhibitor III | 0.75893 | 0.6269792 | 0.8972026 | 0.5424619 | 0.6777495 | 0.8295482 | 1.1217235 |
| 60S ribosomal protein L6 | 0.8693781 | 0.8577501 | 0.9208508 | 0.8547559 | 0.8843828 | 0.7491214 | 0.8032374 |
| Phase-1 RCT-117 | 1.0282917 | 0.6518031 | 1.2029939 | 1.4547267 | 1.2679188 | 1.077324 | 1.385984 |
| Phase-1 RCT-102 | 0.9046398 | 1.010012 | 0.7661107 | 0.6585602 | 0.6796662 | 0.6052773 | 0.7188402 |
| 3-hydroxyisobutyrate dehydrogenase | 0.9688394 | 0.8325859 | 0.8599535 | 0.7912201 | 0.8604622 | 1.3124928 | 1.1780739 |
| ATPase inhibitor (rat mitochondrial IF1 | 0.840812 | 0.7839512 | 0.7433301 | 0.8025259 | 0.984867 | 0.7046531 | 0.7264299 |
| protein) | | | | | | | |
| Alpha-2-macroglobulin | 0.932054 | 0.9609988 | 1.3777124 | 0.6449843 | 0.7689934 | 0.6069262 | 0.7381644 |
| Phase-1 RCT-137 | 0.8579893 | 0.8190631 | 0.8078339 | 0.7396754 | 0.8520518 | 0.6888387 | 0.7043613 |
| Phase-1 RCT 252 | 0.8744342 | 0.785656 | 0.6694077 | 0.7388598 | 0.874507 | 0.7346535 | 0.9573153 |
| Phase-1 RCT-65 | 0.9494342 | 0.9457684 | 0.9848874 | 1.0945808 | 1.0576674 | 2.2428832 | 1.6070827 |
| Liver fatty acid binding protein | 0.8121716 | 1.03972 | 0.9640737 | 0.8452461 | 0.9177136 | 0.6348496 | 0.7400168 |
| Carbamyl phosphate synthetase I | 0.9309803 | 1.1402634 | 0.6478105 | 0.681358 | 0.8882811 | 1.041534 | 0.9567642 |
| c-jun | 1.0756098 | 1.225046 | 1.1570524 | 1.4257243 | 1.2189103 | 1.9942085 | 1.5361661 |
| Transthyretin | 0.7541425 | 0.7861152 | 0.8819279 | 0.494745 | 0.6116468 | 0.8924196 | 0.8428356 |
| Aflatoxin B1 aldehyde reductase | 0.9374989 | 0.6650009 | 0.7657689 | 0.6272773 | 0.6674655 | 0.7779797 | 0.7322443 |
| Phase-1 RCT-15 | 0.96051 | 0.9881148 | 1.0614733 | 0.8990197 | 0.9846166 | 1.8142674 | 1.5162483 |
| Insulin-like growth factor I | 0.7861741 | 1.2405487 | 1.0585281 | 0.8344616 | 1.0034001 | 0.8054617 | 1.0543294 |
| Sodium/bile acid cotransporter | 1.1184043 | 0.8226817 | 0.9371352 | 0.7228838 | 0.7265291 | 0.9814765 | 1.075808 |
| Organic cation transporter 3 | 0.9441845 | 0.970784 | 0.9929735 | 0.9953243 | 0.9190382 | 0.7656504 | 0.7596768 |
| Gadd153 | 0.9670681 | 1.4260406 | 1.069338 | 1.1542411 | 1.1382079 | 1.0789173 | 1.1404984 |
| Phase-1 RCT-109 | 0.9883692 | 1.0069788 | 1.0628793 | 1.1692456 | 1.0970207 | 0.9470659 | 1.0204607 |
| Phase-1 RCT-88 | 1.0301274 | 1.017978 | 0.9779307 | 0.9689238 | 0.9858188 | 0.845895 | 1.01285 |
| Ubiquitin conjugating enzyme (RAD 6 | 0.9364752 | 0.9093629 | 0.8297358 | 0.9994317 | 1.0233289 | 0.8599241 | 0.8106838 |
| homologue) | | | | | | | |
| Glucose-regulated protein 78 | 0.8811379 | 1.042488 | 0.9455944 | 1.2502782 | 1.4777894 | 0.7003085 | 0.8238275 |
| Ribosomal protein L13A | 1.0184661 | 1.0735252 | 1.1131294 | 1.1947376 | 1.1418567 | 1.1540726 | 1.1774577 |
| Monocyte chemotactic protein receptor | 0.9480851 | 1.0324234 | 1.0243238 | 1.1248039 | 1.0786991 | 0.9391533 | 0.9431151 |
| (CCR2) | | | | | | | |
| Fas antigen | 0.981992 | 1.0922645 | 1.0220205 | 1.1587229 | 1.1620443 | 0.9737341 | 1.0831752 |
| Calpactin I heavy chain | 1.0740087 | 1.2154534 | 1.0114828 | 1.0947686 | 1.10403 | 1.2683667 | 1.1953115 |
| Uncoupling protein 2 | 1.1059641 | 1.0662643 | 1.0604824 | 1.1608907 | 1.2703401 | 1.2210954 | 0.9416365 |
| Phase-1 RCT-168 | 0.9626336 | 1.0635265 | 1.126018 | 1.0954902 | 0.945425 | 0.8252074 | 1.0295792 |
| Phase-1 RCT-154 | 1.0677317 | 1.0480802 | 0.8881344 | 0.9567617 | 0.9810448 | 0.8264685 | 0.9249573 |
| Superoxide dismutase Mn | 1.2121351 | 1.1491693 | 1.2256105 | 1.2292718 | 1.1217158 | 1.5066656 | 1.2944945 |
| Phase-1 RCT-214 | 1.0181944 | 0.9996913 | 1.1806769 | 0.9523084 | 1.0404435 | 0.8296471 | 1.0776668 |
| Phase-1 RCT-225 | 1.1336652 | 1.1860988 | 1.3184081 | 0.8641732 | 0.8641053 | 0.5446528 | 1.5560852 |
| Phase-1 RCT-181 | 0.9770507 | 0.9900698 | 0.9814629 | 1.0984005 | 1.080332 | 0.8454187 | 1.1616501 |
| Nucleoside diphosphate kinase beta | 1.0175697 | 1.0929716 | 1.119614 | 1.24368 | 1.2331465 | 1.1546963 | 1.1436331 |
| isoform | | | | | | | |
| Extracellular-signal-regulated kinase 1 | 1.0059124 | 0.784579 | 0.6404572 | 0.4510082 | 0.7103636 | 1.5364091 | 1.3421956 |
| {Ribosomal protein L6} | 0.953007 | 0.8621512 | 0.9142078 | 0.9051589 | 0.8920639 | 1.2932541 | 1.1607488 |
| Aldehyde dehydrogenase, microsomal | 0.9930796 | 1.1237926 | 1.0857381 | 0.9387663 | 0.9761251 | 0.7587779 | 1.2963977 |
| p53 | 1.0084511 | 1.0834016 | 0.9963074 | 0.9395824 | 0.9702871 | 0.8046296 | 0.8259438 |
| Phase-1 RCT-239 | 0.8877506 | 1.1310729 | 0.9956266 | 0.9545893 | 1 | 1.5003866 | 1.5613377 |
| Macrophage inflammatory protein-2 alpha | 0.9507828 | 0.9532023 | 1.0361364 | 1.0484562 | 1.1070929 | 1.2999144 | 1.1258103 |
| IgE binding protein | 1.0171276 | 0.9485236 | 0.9512802 | 0.9884548 | 1.0912433 | 0.9948486 | 1.0022893 |
| Phase-1 RCT-205 | 1.0224509 | 1.0291259 | 0.9850021 | 0.975351 | 0.9961056 | 0.8160384 | 0.976715 |
| Melanoma-associated antigen ME491 | 0.9245447 | 1.0246388 | 0.9541271 | 1.001422 | 1.1038555 | 0.8129706 | 1.0895994 |
| Phase-1 RCT-242 | 1.0514591 | 1.1488353 | 0.981539 | 1.0646199 | 1.0025971 | 0.8915231 | 0.8530104 |
| Enolase alpha | 0.9057663 | 0.6683755 | 1.0121108 | 0.8874027 | 1.0076715 | 1.0187789 | 0.9595807 |
| Thymosin beta-10 | 1.091519 | 1.1393633 | 0.9748127 | 1.0806097 | 1.0975269 | 0.9104219 | 0.9211121 |
| Cofilin | 1.0633426 | 1.0094491 | 1.0079721 | 0.886448 | 0.9549117 | 0.990248 | 1.1728666 |
| Carbonyl reductase | 1.0167141 | 0.9993196 | 1.0549412 | 1.1399708 | 1.0944912 | 0.8984652 | 0.9817401 |
| MAP kinase kinase | 1.025793 | 1.029203 | 1.0442379 | 1.0665947 | 1.0623115 | 1.0542889 | 1.0260231 |
| Phase-1 RCT-162 | 0.9125672 | 0.873972 | 0.9920635 | 1.0828221 | 0.9040207 | 1.0237238 | 1.0383692 |
| Phase-1 RCT-72 | 0.9248243 | 0.7968872 | 0.885241 | 0.9519028 | 0.8610436 | 1.1842612 | 0.8640371 |
| Ribosomal protein S8 | 0.863879 | 0.8119333 | 0.8912815 | 0.9501097 | 0.9026942 | 1.4742512 | 1.0810944 |
| Proliferating cell nuclear antigen gene | 0.9042031 | 1.0339168 | 1.0173527 | 1.0289015 | 1.0905579 | 0.8502681 | 0.8353219 |
| Phase-1 RCT-192 | 0.9028577 | 0.9759845 | 0.9261076 | 0.8834555 | 1.0295821 | 1.057005 | 1.0954647 |
| IkB-a | 0.9152643 | 0.7703335 | 0.9966945 | 0.8217463 | 0.9168354 | 1.0282683 | 1.0467517 |
| Endogenous retroviral sequence, 5′ and | 1.1640471 | 1.1201609 | 1.1097418 | 0.9736523 | 1.0721242 | 0.9431126 | 1.1513983 |
| 3′ LTR | | | | | | | |
| Phase-1 RCT-37 | 0.9560975 | 0.8897503 | 0.9505534 | 0.9788421 | 0.9580015 | 1.3863744 | 1.081175 |
| Matrix metalloproteinase-1 | 0.9287229 | 0.7570963 | 0.9123982 | 1.0203642 | 0.9645019 | 1.1953765 | 1.1805363 |
| Cyclin G | 0.9591736 | 1.1060354 | 1.0818172 | 1.2356483 | 1.124638 | 1.0739167 | 1.0439327 |
| DNA polymerase beta | 0.9258945 | 0.833943 | 0.9665812 | 1.0269709 | 0.9435389 | 0.9003359 | 1.0519295 |
| Phase-1 RCT-127 | 1.0066041 | 0.8099392 | 1.0348914 | 1.2013568 | 1.1187881 | 0.8379202 | 0.9330941 |
| Multidrug resistant protein-2 | 0.998189 | 0.9894818 | 0.9974374 | 0.9573132 | 0.968295 | 1.6365651 | 1.3122982 |
| Ribosomal protein S9 | 0.9434375 | 1.0869831 | 0.7874299 | 1.0072409 | 1.015663 | 0.7851292 | 0.7507926 |
| Phase-1 RCT-12 | 1.0124675 | 1.0252539 | 1.0855843 | 1.1710409 | 1.1247035 | 1.5624273 | 1.8147745 |
| Beta-tubulin, class I | 0.9580359 | 0.970106 | 1.0260394 | 1.2702682 | 1.1306348 | 1.7187997 | 1.79074 |
| Argininosuccinate lyase | 0.9918819 | 1.1279936 | 1.0892242 | 1.0297531 | 1.0519974 | 1.7690759 | 1.7744771 |
| Elongation factor-1 alpha | 0.8673294 | 0.794615 | 1.0254552 | 0.8096444 | 0.9443545 | 0.9174719 | 1.0937097 |
| Phase-1 RCT-55 | 0.7844067 | 0.7953624 | 0.9485411 | 1.1069722 | 0.8209959 | 1.3268735 | 0.9233556 |
| Macrophage inflammatory protein-1 alpha | 0.9491265 | 1.0068731 | 0.936253 | 1.141063 | 1.0875758 | 0.9544671 | 0.9305776 |
| c-H-ras | 1.2330941 | 0.9378442 | 1.1203698 | 1.1520313 | 1.2075806 | 1.1661129 | 1.1455288 |
| Multidrug resistant protein-1 | 1.0066806 | 1.0287322 | 1.0514964 | 1.0453125 | 1.0121155 | 1.4858279 | 1.5504433 |
| T-cell cyclophilin | 0.9139574 | 0.7803552 | 0.8895077 | 0.8481591 | 0.8428069 | 0.8290817 | 0.9431047 |
| Phase-1 RCT-185 | 1.0391177 | 0.781726 | 0.7380878 | 0.6851133 | 0.704472 | 1.0009716 | 0.8795344 |
| 25-DX | 1.0736547 | 0.9270952 | 0.9461273 | 0.8162552 | 0.9450352 | 0.8780577 | 0.8876103 |
| Ref-1 | 1.1454638 | 0.8723422 | 0.9507926 | 1.0392388 | 0.9950004 | 1.2816117 | 1.0300591 |
| Apolipoprotein CIII | 1.1204582 | 1.171068 | 1.0090351 | 0.7459483 | 0.819121 | 0.6452451 | 0.7144027 |
| NADPH quinone oxidoreductase-1 (DT- | 1.1796496 | 0.8599967 | 1.0274825 | 1.1945987 | 1.2820841 | 1.3479934 | 1.3606912 |
| diaphorase) | | | | | | | |
| Pyruvate kinase, muscle | 0.9602091 | 0.903871 | 0.948049 | 0.960112 | 0.9609635 | 1.4318986 | 1.1988251 |
| | STRZ | STRZ | STRZ | STRZ | TAM | TAM | TAM |
| Compound-Dose (2) | 20 | 75 | 75 | 75 | 50 | 50 | 50 |
| Animal Number (3) | 1726 | 1734 | 1735 | 1736 | 1444 | 1445 | 1446 |
| Liver Toxicity Necrosis Classification (4) | no | no | no | no | no | no | no |
| Gene Name (5) | | | | | | | |
| Phase-1 RCT-78 | 1.0641253 | 1.1587545 | 1.0080537 | 1.0137249 | 0.9278859 | 0.9481537 | 1.0250294 |
| Gamma-actin, cytoplasmic | 1.1053953 | 1.3470589 | 1.5195925 | 1.265482 | 1.1163969 | 1.1396797 | 0.9929116 |
| Matrin F/G | 1.6683137 | 1.5621222 | 1.7905936 | 1.4665267 | 1.3033671 | 1.6091503 | 2.165461 |
| Zinc finger protein | 0.9849331 | 1.1290588 | 0.8611186 | 0.8593413 | 0.8490488 | 0.9198084 | 0.7149463 |
| Cathepsin L, sequence 2 | 0.9055718 | 1.4306892 | 1.1899192 | 0.8056619 | 0.5541354 | 0.4778928 | 0.5572593 |
| Gadd45 | 0.8853999 | 0.9540561 | 1.1991626 | 0.8247789 | 1.1405818 | 1.2552423 | 1.4569962 |
| Phase-1 RCT-144 | 0.9378998 | 0.9421235 | 0.7986191 | 1.0626862 | 1.1332371 | 1.0754018 | 0.8684932 |
| Phase-1 RCT-145 | 0.9747712 | 0.8209758 | 0.8681598 | 0.9433557 | 1 | 1.0009544 | 0.9350763 |
| Phase-1 RCT-50 | 1.0227423 | 0.8354489 | 0.8142384 | 0.8932177 | 1.0209101 | 0.9537049 | 0.9577762 |
| Phase-1 RCT-92 | 1.0568192 | 1.2711844 | 0.962109 | 1.3174045 | 0.8363413 | 1.0236319 | 0.8701373 |
| Phase-1 RCT-89 | 1.0077924 | 1.4771751 | 1.3149458 | 1.201048 | 0.9079235 | 0.7999156 | 0.8474186 |
| 14-3-3 zeta | 1.3281392 | 1.2118232 | 1.3416649 | 1.4342045 | 1.26242 | 0.9885881 | 1.1917552 |
| Dynamin-1 (D100) | 0.9692766 | 0.7540751 | 0.8819521 | 0.95436 | 1.10437 | 1.1078846 | 1.1650732 |
| Insulin-like growth factor binding protein 1 | 1.4227346 | 1.4674399 | 1.3085507 | 1.0652021 | 1.4124533 | 1.7562249 | 1.4516193 |
| L-gulono-gamma-lactone oxidase | 1.0544437 | 1.0589004 | 1.2483023 | 0.9609066 | 0.622203 | 0.3910599 | 0.6455209 |
| Ornithine decarboxylase | 1.1287411 | 1.0679945 | 1.0621027 | 1.4438003 | 1.2171279 | 1.1721662 | 1.0575385 |
| PAR interacting protein | 1.0290865 | 1.0211519 | 0.9991555 | 1.1575786 | 1.1711416 | 1.1117691 | 0.9631572 |
| Phase-1 RCT-128 | 0.9548848 | 1.1235951 | 1.018373 | 1.0658041 | 1.1131978 | 0.9948149 | 1.3001161 |
| Phase-1 RCT-180 | 1.0529449 | 0.9915967 | 1.0810274 | 1.1543093 | 0.9090425 | 1.0307662 | 0.7706834 |
| Phase-1 RCT-182 | 0.9677103 | 1.2275738 | 0.9116788 | 0.7936931 | 0.8728008 | 0.8199645 | 0.9107778 |
| Phase-1 RCT-207 | 1.0913901 | 1.0621737 | 1.0829772 | 1.1566118 | 1.0936779 | 1.0143628 | 0.6763396 |
| Phase-1 RCT-213 | 1.0457737 | 1.023188 | 1.0469928 | 1.22459 | 0.9916373 | 0.9062256 | 0.8650281 |
| Phase-1 RCT-256 | 1.0735602 | 1.0020328 | 1.2191371 | 1.0516664 | 1.200487 | 1.1759692 | 1.428323 |
| Phase-1 RCT-258 | 0.9804647 | 0.8360432 | 0.9377824 | 1.1221821 | 0.9851441 | 1.0022857 | 0.6763822 |
| Phase-1 RCT-264 | 1.1643256 | 1.8084167 | 1.500347 | 1.2399265 | 0.6173751 | 0.4431404 | 0.9288056 |
| Phase-1 RCT-271 | 1.0906459 | 1.0445526 | 0.9675962 | 1.3313634 | 0.8872891 | 0.8355968 | 1.0379547 |
| Phase-1 RCT-288 | 0.8595554 | 0.9659523 | 0.9140872 | 1.1160909 | 0.7140315 | 0.7605865 | 0.8446645 |
| Phase-1 RCT-38 | 1.0588715 | 1.1786852 | 1.2169982 | 1.0385798 | 1.2209257 | 1.1390448 | 1.4148434 |
| Phase-1 RCT-39 | 1.0215226 | 0.9066243 | 0.9549679 | 0.9286254 | 1.1317202 | 0.9755704 | 0.9963913 |
| Phase-1 RCT-68 | 1.1382263 | 1.0011362 | 1.011726 | 0.9167155 | 0.9859036 | 0.9088465 | 1.1110419 |
| Phase-1 RCT-33 | 0.929318 | 1.1451622 | 1.0576588 | 0.7382163 | 1.0577747 | 1.1971983 | 1.2020265 |
| Phase-1 RCT-36 | 1.0139662 | 0.8988149 | 0.9188324 | 0.8770679 | 1.0750036 | 1.0848112 | 1.0631124 |
| 17-beta hydroxysteroid dehydrogenase, | 0.9466634 | 1.404402 | 0.6100983 | 0.5128151 | 0.5686511 | 1.8830171 | 1.996333 |
| type 2 | | | | | | | |
| Senescence marker protein-30 | 0.797487 | 1.0540508 | 1.5200514 | 1.0492301 | 0.6208186 | 0.5858042 | 0.664825 |
| Ribosomal protein S17 | 0.8036066 | 1.4218715 | 1.1841551 | 0.8061634 | 1.0041697 | 0.945447 | 0.9754946 |
| Phase-1 RCT-83 | 1.0033101 | 1.6773357 | 1.2913413 | 1.0602082 | 0.8948577 | 1.1997105 | 1.1679456 |
| Phase-1 RCT-49 | 0.9297721 | 0.7637004 | 0.7696008 | 0.7513378 | 1.066205 | 1.0563948 | 0.968748 |
| Phase-1 RCT-48 | 1.1029165 | 1.0747241 | 1.5240566 | 1.5266652 | 0.8194929 | 0.8529981 | 0.8886098 |
| Phase-1 RCT-40 | 1.1051462 | 1.5968108 | 1.3704922 | 1.129883 | 0.7253522 | 0.8760331 | 0.9518535 |
| Phase-1 RCT-296 | 0.9849607 | 0.994139 | 0.8108041 | 0.8716732 | 0.8737515 | 0.9325896 | 0.9186434 |
| Phase-1 RCT-295 | 1.2403063 | 1.4447078 | 1.2571138 | 1.0267018 | 1.0479021 | 0.9194485 | 1.0475874 |
| Phase-1 RCT-291 | 0.8712271 | 1.2252909 | 0.9997348 | 0.8335792 | 0.9789052 | 0.9685076 | 1.1316181 |
| Phase-1 RCT-270 | 0.9734337 | 1.3284816 | 1.1876316 | 1.2161351 | 1.1075922 | 1.3286765 | 1.2588646 |
| Phase-1 RCT-241 | 0.9198793 | 0.7921698 | 0.9096025 | 0.984166 | 1.2144864 | 0.9397647 | 0.7625151 |
| Phase-1 RCT-191 | 1.5967498 | 1.7349277 | 1.466132 | 1.6392224 | 1.2335364 | 1.3052152 | 1.3182726 |
| Phase-1 RCT-189 | 1.0497507 | 1.0349255 | 1.0374464 | 1.129152 | 0.7520812 | 0.8217855 | 1.0086601 |
| Phase-1 RCT-179 | 1.0210108 | 0.953229 | 1.0280899 | 1.2578241 | 0.8873519 | 0.8380398 | 0.9123146 |
| Phase-1 RCT-152 | 0.8514479 | 1.1782383 | 1.0025393 | 0.8116688 | 1.0241166 | 1.1132532 | 1.0760983 |
| Phase-1 RCT-123 | 0.9430606 | 0.7467774 | 0.8518115 | 0.8713485 | 1.038028 | 0.890298 | 1.104524 |
| Paraoxonase 1 | 0.7937692 | 1.4776933 | 1.1580447 | 0.8590479 | 0.5466783 | 0.6677387 | 0.6813728 |
| Organic anion transporter 3 | 1.1766984 | 1.5992687 | 1.5729119 | 1.0179522 | 0.8815925 | 0.7192455 | 0.7567985 |
| N-hydroxy-2-acetylaminofluorene | 0.8607351 | 1.5697435 | 1.1276206 | 1.024573 | 0.742498 | 0.6612911 | 0.7870834 |
| sulfotransferase (ST1C1) | | | | | | | |
| Integrin beta1 | 1.060605 | 0.8670968 | 1.0306348 | 1.0033777 | 1.3250773 | 1.3765707 | 1.3333787 |
| Insulin-like growth factor binding protein 3 | 0.9547393 | 1.060828 | 1.3656591 | 1.2388474 | 0.6898214 | 0.7488388 | 0.7994039 |
| ID-1 | 1.2225031 | 0.9188454 | 1.0686537 | 1.2654868 | 1.2670076 | 1.2200742 | 1.0457174 |
| Hepatic lipase | 0.8315541 | 0.8980203 | 0.8423517 | 0.9715542 | 1.0209522 | 0.9878028 | 0.9895889 |
| Heme oxygenase | 0.9968744 | 1.2814184 | 1.0916878 | 0.9411351 | 0.8965741 | 0.756523 | 0.7813662 |
| Equilbrative nitrobenzylthioinosine- | 1.0225239 | 1.3581177 | 1.0764098 | 1.0087327 | 0.8120691 | 0.8299047 | 0.9187925 |
| sensitive nucleoside transporter | | | | | | | |
| Epidermal growth factor | 0.8170523 | 0.8413999 | 0.8933677 | 0.943271 | 1.2587658 | 1.254917 | 1.3513049 |
| c-myc | 1.1125144 | 1.0040494 | 0.9078854 | 0.737479 | 1.2144463 | 1.1787544 | 1.054029 |
| Carbonic anhydrase III | 1.3699516 | 1.7898778 | 1.5241112 | 1.7230647 | 0.8151803 | 0.3897834 | 0.9930639 |
| Beta-actin | 2.1953273 | 1.7297508 | 1.8680906 | 2.549134 | 0.8364801 | 0.8057398 | 0.8272831 |
| Bax (alpha) | 1.5562704 | 1.2511672 | 1.2055025 | 1.0271983 | 1.0757004 | 0.5099394 | 0.6888019 |
| Alpha 1 - inhibitor III | 1.0052762 | 1.5909926 | 1.303538 | 1.4185504 | 0.6899033 | 0.6791835 | 0.8462793 |
| 60S ribosomal protein L6 | 0.8412206 | 0.7798118 | 0.8616588 | 0.898274 | 0.9449241 | 0.8584647 | 1.0307605 |
| Phase-1 RCT-117 | 0.9890829 | 0.764007 | 0.7163424 | 0.8907313 | 1.0904379 | 0.8528352 | 0.8779927 |
| Phase-1 RCT-102 | 0.6603475 | 0.6394128 | 1.0148526 | 1.045564 | 0.7732715 | 0.4118237 | 0.3453793 |
| 3-hydroxyisobutyrate dehydrogenase | 1.0021641 | 1.355593 | 1.2733837 | 1.0233724 | 0.6739355 | 0.9116629 | 1.0939108 |
| ATPase inhibitor (rat mitochondrial IF1 | 0.6989516 | 0.9941211 | 0.9522879 | 1.1653461 | 0.8073878 | 0.6449877 | 0.9184261 |
| protein) | | | | | | | |
| Alpha-2-macroglobulin | 0.7293021 | 0.9087332 | 1.0215987 | 1.5481827 | 0.7403203 | 0.8920128 | 1.0061696 |
| Phase-1 RCT-137 | 0.7409365 | 0.9633327 | 0.8127893 | 0.8963729 | 1.2298855 | 1.3277155 | 1.3151113 |
| Phase-1 RCT 252 | 0.8689532 | 1.3197145 | 1.1085185 | 0.8196681 | 0.8091438 | 0.6500632 | 0.8962208 |
| Phase-1 RCT-65 | 1.7984816 | 1.487197 | 1.6407164 | 1.671033 | 1.1258929 | 1.1501768 | 1.3870736 |
| Liver fatty acid binding protein | 0.6184926 | 0.4745234 | 0.8875213 | 0.8680769 | 0.5785438 | 0.6715767 | 0.8783318 |
| Carbamyl phosphate synthetase I | 0.8660225 | 1.4805561 | 1.0973182 | 0.7921361 | 0.7327355 | 0.5996541 | 0.9028696 |
| c-jun | 1.8345221 | 1.6257607 | 2.243964 | 1.2570914 | 1.056444 | 1.4223489 | 1.0999564 |
| Transthyretin | 0.7828351 | 1.2423944 | 1.0576386 | 1.0314531 | 0.4534544 | 0.4112089 | 0.768702 |
| Aflatoxin B1 aldehyde reductase | 0.8220416 | 1.3871316 | 0.9371507 | 0.8744916 | 0.8060822 | 0.9133955 | 1.0332274 |
| Phase-1 RCT-15 | 1.603406 | 1.4217535 | 1.3133403 | 1.3926247 | 0.7530602 | 1.1685673 | 1.3256501 |
| Insulin-like growth factor I | 0.820111 | 0.769081 | 1.1194048 | 0.9581918 | 0.6386777 | 0.8039534 | 0.7728921 |
| Sodium/bile acid cotransporter | 0.9092911 | 1.1376054 | 1.2210947 | 0.8549092 | 0.5457214 | 0.5639889 | 0.6821706 |
| Organic cation transporter 3 | 0.8432896 | 0.6517982 | 0.6969023 | 1.0580842 | 1.0326982 | 1.0040264 | 1.1148312 |
| Gadd153 | 1.1499121 | 0.9868522 | 0.9807586 | 0.9394975 | 1.2024871 | 1.3005846 | 0.9985122 |
| Phase-1 RCT-109 | 1.0680451 | 1.0024991 | 0.9742496 | 0.9225084 | 1.0363775 | 1.1446784 | 1.1651517 |
| Phase-1 RCT-88 | 0.929357 | 0.7766936 | 0.8670317 | 1.0210425 | 0.9071384 | 0.7541308 | 0.599474 |
| Ubiquitin conjugating enzyme (RAD 6 | 0.9266412 | 0.8182187 | 0.9165715 | 0.8824263 | 1.0342103 | 0.9980999 | 1.1338642 |
| homologue) | | | | | | | |
| Glucose-regulated protein 78 | 0.8328461 | 0.5972191 | 1.0788006 | 1.448943 | 1.1650953 | 1.1194891 | 1.2293307 |
| Ribosomal protein L13A | 1.1006299 | 0.8147422 | 0.9426771 | 1.0894018 | 1.0544791 | 1.1076297 | 1.1340872 |
| Monocyte chemotactic protein receptor | 1.0746467 | 0.7033113 | 0.3177289 | 0.9158629 | 1.0793685 | 1.074968 | 1.0898907 |
| (CCR2) | | | | | | | |
| Fas antigen | 1.0543703 | 0.8300309 | 1.2039263 | 1.2438197 | 0.9458168 | 1.1028961 | 1.0973803 |
| Calpactin I heavy chain | 1.0938904 | 0.8853396 | 0.9736849 | 1.0782111 | 0.9418693 | 1.0414556 | 1.2792966 |
| Uncoupling protein 2 | 1.2521491 | 0.9943355 | 1.0608095 | 1.1882671 | 0.9566727 | 0.9876731 | 1.0644075 |
| Phase-1 RCT-168 | 1.1433146 | 1.1433203 | 1.0375334 | 1.1597106 | 0.9747601 | 1.6787091 | 1.4312359 |
| Phase-1 RCT-154 | 0.9491705 | 0.8559869 | 0.9516622 | 1.1172873 | 1.0307691 | 0.9225909 | 1 |
| Superoxide dismutase Mn | 1.2167249 | 1.2946608 | 1.2880602 | 1.1104064 | 1.1302496 | 1.2395401 | 1.1978778 |
| Phase-1 RCT-214 | 1.0704232 | 0.9660404 | 0.9806888 | 1.1809413 | 0.9210545 | 0.9676013 | 1.1710646 |
| Phase-1 RCT-225 | 1.5745536 | 1.3161542 | 1.0986937 | 1.4600333 | 1.1418269 | 1.0364295 | 0.9894386 |
| Phase-1 RCT-181 | 0.8551773 | 0.8840938 | 1.3747162 | 0.9190233 | 0.9909251 | 0.9245121 | 0.7984807 |
| Nucleoside diphosphate kinase beta | 1.0135412 | 1.2564354 | 1.0700525 | 1.1338161 | 1.4175729 | 1.5603817 | 1.6332768 |
| isoform | | | | | | | |
| Extracellular-signal-regulated kinase 1 | 1.6605065 | 1.844691 | 1.604055 | 1.4990138 | 0.9135842 | 0.7602873 | 1.1938863 |
| {Ribosomal protein L6} | 1.0629187 | 1.2991239 | 1.2546222 | 0.9363658 | 1.0874481 | 0.9721299 | 1.1312357 |
| Aldehyde dehydrogenase, microsomal | 1 | 0.8723847 | 1.0815754 | 1.3593597 | 0.888068 | 0.8521541 | 1.0746406 |
| p53 | 0.9428068 | 0.748112 | 0.8414866 | 1.0803164 | 1.0822004 | 1.0618148 | 1.04403 |
| Phase-1 RCT-239 | 1.1344893 | 0.812318 | 0.9850034 | 1.1478316 | 0.8662066 | 1.0461627 | 1.4891381 |
| Macrophage inflammatory protein-2 alpha | 1.200188 | 0.9946365 | 0.9963663 | 0.7764819 | 1.0952287 | 1.2709835 | 1.1070485 |
| IgE binding protein | 0.9389134 | 0.8394344 | 0.8658152 | 0.8705272 | 0.9813699 | 1.137184 | 1.0424714 |
| Phase-1 RCT-205 | 1.0291564 | 0.9717954 | 0.9410456 | 1.0489849 | 1.1917849 | 1.0237417 | 1.0299168 |
| Melanoma-associated antigen ME491 | 0.9302378 | 1.0669028 | 0.852495 | 0.8111453 | 0.9690505 | 0.8992939 | 0.8069743 |
| Phase-1 RCT-242 | 0.9644516 | 0.8024417 | 0.8459864 | 0.9224523 | 1.39055 | 1.2406824 | 0.9382833 |
| Enolase alpha | 1.0598918 | 1.474059 | 1.3089176 | 1.1038827 | 1.3091049 | 1.6978949 | 1.4478686 |
| Thymosin beta-10 | 0.9949377 | 1.0273925 | 0.9728416 | 1.2153684 | 0.8790188 | 0.7999874 | 1.0447135 |
| Cofilin | 1.0572165 | 1.1250281 | 1.0262122 | 1.3416705 | 0.6973916 | 0.8512248 | 1.0178592 |
| Carbonyl reductase | 1.104053 | 0.8036659 | 0.8692865 | 0.9802389 | 1.0604358 | 1.1759313 | 1.166122 |
| MAP kinase kinase | 0.8441146 | 1.0229635 | 0.9517219 | 0.8363824 | 1.0452118 | 1.1314787 | 1.0612104 |
| Phase-1 RCT-162 | 1.0052191 | 1.0085845 | 1.1512847 | 0.9939437 | 0.8941578 | 0.9526535 | 0.8753125 |
| Phase-1 RCT-72 | 0.8762482 | 0.7452941 | 0.7772508 | 0.8626437 | 0.9976727 | 0.9864937 | 0.9102406 |
| Ribosomal protein S8 | 0.8903742 | 1.230868 | 1.2828728 | 0.9614086 | 0.8807082 | 0.9972574 | 0.9960154 |
| Proliferating cell nuclear antigen gene | 0.9261123 | 0.7709491 | 0.8301197 | 0.7680544 | 1.2398424 | 1.1334608 | 1.0114144 |
| Phase-1 RCT-192 | 1.0029683 | 0.9768201 | 0.9981045 | 1.0763005 | 1.0045581 | 0.8836762 | 1.1551164 |
| IkB-a | 0.9837572 | 1.4217272 | 1.1352712 | 1.0878161 | 1.3144373 | 1.3327402 | 1.3635526 |
| Endogenous retroviral sequence, 5′ and | 1.1397002 | 1.2980335 | 1.1588854 | 0.9709129 | 1.1878352 | 0.9295333 | 1.2856004 |
| 3′ LTR | | | | | | | |
| Phase-1 RCT-37 | 1.0568938 | 0.9879192 | 1.004018 | 0.9553637 | 1.0673664 | 0.9238964 | 1.031145 |
| Matrix metalloproteinase-1 | 1.2635779 | 1.334484 | 1.2477559 | 1.1385233 | 0.8169956 | 0.9118285 | 1.0316042 |
| Cyclin G | 1.0266666 | 1.024409 | 0.8748565 | 1.0296954 | 1.3617098 | 1.0236127 | 0.8698508 |
| DNA polymerase beta | 0.9624347 | 1.0583605 | 1.0566417 | 1.2219576 | 1.027809 | 1.1366084 | 1.0689051 |
| Phase-1 RCT-127 | 0.9073644 | 0.7691994 | 0.8795638 | 0.8993906 | 1.1169164 | 0.7851216 | 0.8287935 |
| Multidrug resistant protein-2 | 1.4545772 | 1.4644339 | 1.4995793 | 1.1205508 | 0.8736591 | 0.7705308 | 0.6886776 |
| Ribosomal protein S9 | 0.8315102 | 0.7200231 | 0.9332525 | 0.9533446 | 0.9348049 | 1.027136 | 1.136326 |
| Phase-1 RCT-12 | 1.6903733 | 1.4308733 | 1.381364 | 1.6210648 | 1.2306056 | 1.2250264 | 1.2145756 |
| Beta-tubulin, class I | 1.6202523 | 1.742921 | 1.6057833 | 1.9970199 | 1.3353655 | 1.3223321 | 1.3134574 |
| Argininosuccinate lyase | 1.744718 | 1.77208 | 1.5136764 | 1.3133042 | 0.6569269 | 0.8342571 | 0.9403848 |
| Elongation factor-1 alpha | 0.9490117 | 1.1682907 | 1.1774753 | 1.0589889 | 0.791445 | 0.9045323 | 0.9612051 |
| Phase-1 RCT-55 | 0.9198794 | 0.7186564 | 0.8209871 | 0.9124084 | 1.109773 | 0.950522 | 0.8013769 |
| Macrophage inflammatory protein-1 alpha | 1.0237983 | 0.8641072 | 0.8634783 | 0.822688 | 1.2855313 | 1.1670967 | 1.323286 |
| c-H-ras | 1.1605514 | 1.0756544 | 1.0217382 | 1.0594269 | 1.060263 | 0.9604461 | 1.1278541 |
| Multidrug resistant protein-1 | 1.6577082 | 1.4090886 | 1.5464647 | 1.159337 | 1.0745623 | 1.0417026 | 0.8778212 |
| T-cell cyclophilin | 0.9261271 | 1.0242454 | 0.9833316 | 1.3078314 | 0.9531856 | 0.9498904 | 1.114517 |
| Phase-1 RCT-185 | 0.9432334 | 1.5356647 | 1.0488269 | 0.9786034 | 0.8147798 | 0.7913981 | 0.9331284 |
| 25-DX | 0.9207166 | 0.8763208 | 0.9455249 | 1.2204585 | 0.9364788 | 0.9173096 | 1.0403676 |
| Ref-1 | 1.0906405 | 0.9869899 | 0.8419995 | 0.8251507 | 1.3904282 | 1.0614333 | 1.0732073 |
| Apolipoprotein CIII | 0.868214 | 0.9235063 | 0.794842 | 1.0144042 | 0.9728832 | 0.8539191 | 1.0312399 |
| NADPH quinone oxidoreductase-1 (DT- | 1.1846006 | 0.8256438 | 0.9200016 | 0.8214404 | 0.9009331 | 1.2735037 | 1.1858855 |
| diaphorase) | | | | | | | |
| Pyruvate kinase, muscle | 1.239276 | 1.1165819 | 1.1569284 | 1.0312232 | 0.8530294 | 0.9954117 | 1.0507381 |
| | TAM | TAM | TAM | TET | TET | TET | THEO |
| Compound-Dose (2) | 200 | 200 | 200 | 50 | 50 | 50 | 25 |
| Animal Number (3) | 1454 | 1455 | 1456 | 124 | 125 | 126 | 2524 |
| Liver Toxicity Necrosis Classification (4) | no | no | no | no | no | no | no |
| Gene Name (5) | | | | | | | |
| Phase-1 RCT-78 | 0.8199514 | 0.8129773 | 0.7254933 | 0.8395404 | 0.9722026 | 0.9564239 | 1.1118132 |
| Gamma-actin, cytoplasmic | 1.7031103 | 1.2575493 | 1.0774838 | 1.1315999 | 1.053749 | 1.0662037 | 0.8577148 |
| Matrin F/G | 1.3838259 | 0.7349602 | 0.8193156 | 0.7974814 | 0.9038278 | 0.733999 | 1.1405038 |
| Zinc finger protein | 0.856346 | 0.9497678 | 0.7629835 | 1.6210169 | 0.8846312 | 0.9309457 | 0.9977491 |
| Cathepsin L, sequence 2 | 0.8703103 | 1.3191684 | 1.001722 | 1.1904118 | 1.1133437 | 1.078593 | 0.6765338 |
| Gadd45 | 1.3178833 | 1.3832443 | 1.189902 | 1.166446 | 1.2071856 | 1.2554624 | 1.3429922 |
| Phase-1 RCT-144 | 0.9935141 | 1.0238136 | 1.1933059 | 1.1969006 | 0.9762239 | 1.1177388 | 1.0048292 |
| Phase-1 RCT-145 | 1.0473697 | 1.0428458 | 1.2281488 | 0.8980976 | 0.9208353 | 0.94633 | 0.8991386 |
| Phase-1 RCT-50 | 0.8363542 | 1.0581818 | 0.9061382 | 1.087121 | 0.980378 | 1.1532407 | 0.7137178 |
| Phase-1 RCT-92 | 0.7643873 | 0.938776 | 0.7046862 | 0.8762254 | 1.2105176 | 1.0251029 | 1.222995 |
| Phase-1 RCT-89 | 0.6536114 | 0.9911636 | 0.6601166 | 1.0601469 | 0.948076 | 0.9364744 | 1.0379289 |
| 14-3-3 zeta | 1.2847176 | 1.0563912 | 1.1491407 | 1.2425185 | 1.1583624 | 1.2175926 | 0.76079 |
| Dynamin-1 (D100) | 0.9364267 | 1.0420673 | 0.8889 | 1.1568547 | 0.7369262 | 1.2310212 | 0.8634897 |
| Insulin-like growth factor binding protein 1 | 2.1253257 | 2.1676147 | 1.0865926 | 2.0790665 | 1.0016104 | 1.0432521 | 0.7623852 |
| L-gulono-gamma-lactone oxidase | 0.4604853 | 0.4199915 | 0.4691511 | 0.5339329 | 0.7025774 | 0.5285399 | 1.0359339 |
| Ornithine decarboxylase | 1.8062198 | 1.0924271 | 0.9471608 | 1.2921811 | 1.1727189 | 1.1833607 | 1.0720319 |
| PAR interacting protein | 1.0979791 | 1.0126141 | 1.0486286 | 1.116798 | 1.0540023 | 1.1270181 | 0.907394 |
| Phase-1 RCT-128 | 0.9341075 | 0.8950629 | 0.7271665 | 0.8308749 | 0.8511146 | 0.9104533 | 1.0648651 |
| Phase-1 RCT-180 | 0.9091576 | 0.9346528 | 0.973053 | 0.820663 | 0.8729075 | 0.9223328 | 1.0976607 |
| Phase-1 RCT-182 | 0.7489989 | 0.648468 | 0.7205959 | 0.6565755 | 1.0623275 | 0.7939815 | 1.1661099 |
| Phase-1 RCT-207 | 0.847003 | 1.0251136 | 0.9611659 | 0.9303474 | 0.9151595 | 0.7956744 | 0.6940133 |
| Phase-1 RCT-213 | 0.9718465 | 0.9505072 | 1.1022764 | 0.9374555 | 0.8571158 | 0.855327 | 1.124357 |
| Phase-1 RCT-256 | 0.905609 | 0.4972256 | 0.5830324 | 0.5910049 | 0.8416187 | 0.5667403 | 0.9361348 |
| Phase-1 RCT-258 | 0.9239843 | 0.8758999 | 0.8415285 | 0.8719316 | 0.9079191 | 0.9465278 | 0.9930429 |
| Phase-1 RCT-264 | 0.6897354 | 0.327111 | 0.3309729 | 0.338292 | 0.727333 | 0.5220181 | 1.1209896 |
| Phase-1 RCT-271 | 0.7890435 | 0.9856584 | 0.723623 | 1.0286099 | 0.9843827 | 0.8294805 | 0.8939597 |
| Phase-1 RCT-288 | 0.7734847 | 0.8568396 | 0.5801653 | 0.8153569 | 0.9493987 | 1.0067418 | 0.8112981 |
| Phase-1 RCT-38 | 0.9786336 | 0.5295794 | 0.7466947 | 0.5029312 | 0.8175852 | 0.5394413 | 0.9296822 |
| Phase-1 RCT-39 | 1.1614972 | 1.1649044 | 1.3134143 | 1.1153663 | 1.129135 | 1.0298624 | 0.7388039 |
| Phase-1 RCT-68 | 1.1661713 | 0.9773752 | 1.1146749 | 1.0010878 | 0.9493987 | 0.9739088 | 0.9979102 |
| Phase-1 RCT-33 | 0.9440649 | 0.9733916 | 0.6984465 | 0.8846656 | 0.7611661 | 0.9982615 | 1.4083302 |
| Phase-1 RCT-36 | 0.9563578 | 1.0121739 | 0.8265198 | 1.1027036 | 0.9612393 | 1.0856233 | 0.9556724 |
| 17-beta hydroxysteroid dehydrogenase, | 0.3762262 | 0.6186293 | 0.468906 | 0.4633963 | 0.728251 | 0.9774213 | 2.4476786 |
| type 2 | | | | | | | |
| Senescence marker protein-30 | 0.8456749 | 1.1193988 | 0.7999491 | 0.8969482 | 0.9281873 | 0.8445434 | 1.6874626 |
| Ribosomal protein S17 | 1.3845865 | 1.1964302 | 1.40463 | 1 | 1.8109473 | 1.0712252 | 1.2704825 |
| Phase-1 RCT-83 | 1.3266336 | 0.9322078 | 1.0825219 | 0.7713074 | 0.7295451 | 0.7790529 | 0.7493564 |
| Phase-1 RCT-49 | 0.9202781 | 0.9847716 | 1.0089515 | 0.9527288 | 1.027578 | 1.0516976 | 1.2191465 |
| Phase-1 RCT-48 | 0.7765077 | 0.7227169 | 0.7559468 | 0.7794604 | 0.731387 | 0.8033215 | 1.2694908 |
| Phase-1 RCT-40 | 0.9525846 | 0.9125604 | 0.6989991 | 0.9857869 | 0.9193589 | 0.9519676 | 1.2052593 |
| Phase-1 RCT-296 | 0.4647107 | 0.1352426 | 0.3842189 | 0.735109 | 1.1982679 | 1.115162 | 0.9515432 |
| Phase-1 RCT-295 | 1.1535925 | 0.9654422 | 0.9683782 | 0.7689385 | 0.7500757 | 0.822817 | 1.300492 |
| Phase-1 RCT-291 | 0.8774033 | 1.033975 | 0.7979888 | 0.7452951 | 0.9820863 | 0.9999139 | 1.2453846 |
| Phase-1 RCT-270 | 0.807826 | 0.9569037 | 1.054815 | 0.6542483 | 0.8770163 | 0.9422358 | 1.128376 |
| Phase-1 RCT-241 | 1.1717186 | 0.9967616 | 1.248113 | 1.1315999 | 0.6622441 | 1.173317 | 0.753805 |
| Phase-1 RCT-191 | 1.0018103 | 0.9247975 | 0.8543958 | 1.2947253 | 0.8502629 | 1.0040449 | 1.1488519 |
| Phase-1 RCT-189 | 0.8621593 | 0.636373 | 0.7199805 | 0.5182719 | 0.7131696 | 0.516619 | 1.2801281 |
| Phase-1 RCT-179 | 1.1622181 | 0.8286291 | 1.1227076 | 0.70288 | 0.9869624 | 1.073728 | 0.7884871 |
| Phase-1 RCT-152 | 1.7324953 | 1.0664618 | 1.5980669 | 1.4661828 | 1.4455384 | 0.9931613 | 1.2835128 |
| Phase-1 RCT-123 | 1.0499054 | 1.0430107 | 0.8665757 | 1.165228 | 1.0978544 | 1.1859434 | 0.7506227 |
| Paraoxonase 1 | 0.5616092 | 0.8349367 | 0.6425852 | 0.5460751 | 0.9099295 | 0.689443 | 1.1134846 |
| Organic anion transporter 3 | 0.7857837 | 1.4937088 | 1.1925417 | 1.5349834 | 1.2177473 | 1.1440436 | 0.6927095 |
| N-hydroxy-2-acetylaminofluorene | 0.822546 | 1.047384 | 0.7346416 | 1.050825 | 0.9882862 | 0.9928346 | 1.1651704 |
| sulfotransferase (ST1C1) | | | | | | | |
| Integrin beta1 | 1.5783515 | 1.2128258 | 1.3669189 | 0.9892299 | 0.6904718 | 1.1376752 | 0.822519 |
| Insulin-like growth factor binding protein 3 | 0.8009314 | 0.5635392 | 0.613178 | 0.5628117 | 0.974935 | 0.6519109 | 1.1858706 |
| ID-1 | 1.2613565 | 1.179812 | 1.383273 | 1.1593479 | 1.1525378 | 1.1378869 | 1.011096 |
| Hepatic lipase | 1.0255903 | 0.9140714 | 0.8114616 | 1.2050674 | 0.8961931 | 0.822174 | 0.9864988 |
| Heme oxygenase | 1.4459096 | 1.1873052 | 1.287448 | 1.9962523 | 1.4924505 | 1.7022004 | 1.3555529 |
| Equilbrative nitrobenzylthioinosine- | 0.9214622 | 1.0908736 | 0.8289661 | 1.0384893 | 0.8604744 | 1.0107527 | 1.0045701 |
| sensitive nucleoside transporter | | | | | | | |
| Epidermal growth factor | 2.1487205 | 1.0922012 | 1.803722 | 0.9463714 | 0.96927 | 1.1534176 | 0.9163413 |
| c-myc | 1.2253023 | 1.0831221 | 1.1514273 | 1.2483193 | 0.7256792 | 1.3030969 | 0.8686571 |
| Carbonic anhydrase III | 0.4534799 | 0.3031558 | 0.4350895 | 0.5062689 | 0.3188921 | 0.5052283 | 1.824037 |
| Beta-actin | 1.1568799 | 0.8151919 | 0.9350786 | 0.6961991 | 0.4796252 | 0.5812667 | 0.9646956 |
| Bax (alpha) | 0.4469287 | 1.1551467 | 0.5194896 | 1.0758749 | 0.8386138 | 0.9943155 | 0.9622003 |
| Alpha 1 - inhibitor III | 0.4112299 | 0.3553737 | 0.3668589 | 0.3990438 | 0.8303577 | 0.4334261 | 0.5835939 |
| 60S ribosomal protein L6 | 1.2534026 | 0.8302401 | 1.3567817 | 0.8240581 | 1.1452229 | 0.9096289 | 1.1626729 |
| Phase-1 RCT-117 | 0.9376597 | 0.9134932 | 1.213156 | 0.8045321 | 0.7207187 | 0.7801098 | 1.5250432 |
| Phase-1 RCT-102 | 0.4588046 | 0.9512948 | 0.4304971 | 1.0819418 | 1.0665034 | 0.8520081 | 0.7791073 |
| 3-hydroxyisobutyrate dehydrogenase | 0.9946187 | 0.9517555 | 0.7363054 | 0.9151687 | 0.9640878 | 0.9877237 | 1.2532411 |
| ATPase inhibitor (rat mitochondrial IF1 | 1.0888848 | 0.8717527 | 0.7911507 | 0.7080303 | 1.0793669 | 0.7753128 | 0.9808198 |
| protein) | | | | | | | |
| Alpha-2-macroglobulin | 0.6086322 | 1.6362113 | 0.8673278 | 1.5497015 | 2.7481077 | 1.3945707 | 0.6836838 |
| Phase-1 RCT-137 | 1.4324815 | 0.9067442 | 1.1420352 | 0.5996904 | 0.9135224 | 0.695694 | 1.2189823 |
| Phase-1 RCT 252 | 0.6148788 | 0.8520103 | 0.510931 | 0.6896634 | 0.7217438 | 0.6767417 | 1.2439872 |
| Phase-1 RCT-65 | 1.1094252 | 1.0667254 | 1.0461574 | 1.0904137 | 0.8678359 | 0.9705321 | 1.2157156 |
| Liver fatty acid binding protein | 0.7253481 | 0.5528908 | 0.4277342 | 0.4093002 | 0.8858043 | 0.5730922 | 1.0042106 |
| Carbamyl phosphate synthetase I | 0.6121652 | 0.8511922 | 0.6115704 | 0.6434848 | 0.7802545 | 0.5541058 | 1.2261547 |
| c-jun | 1.063707 | 1.1387674 | 1.0363014 | 0.9348393 | 0.8071801 | 0.9310234 | 1.2322105 |
| Transthyretin | 0.631299 | 0.2887699 | 0.2858779 | 0.2709392 | 0.6029686 | 0.3738904 | 1.0732299 |
| Aflatoxin B1 aldehyde reductase | 0.8084773 | 0.534627 | 0.9690632 | 0.5364136 | 0.538252 | 0.6043366 | 0.7807789 |
| Phase-1 RCT-15 | 1.2940352 | 1.1343211 | 1.1520113 | 1.1197882 | 0.9388264 | 1.0120585 | 1.0873644 |
| Insulin-like growth factor I | 0.7804681 | 0.47297 | 0.5419979 | 0.4713021 | 0.9080924 | 0.6112455 | 1.2218028 |
| Sodium/bile acid cotransporter | 0.3737859 | 0.9454246 | 0.4663532 | 0.9011838 | 1.0466179 | 0.8405446 | 1.0467303 |
| Organic cation transporter 3 | 1.3939602 | 0.9723004 | 1.4274712 | 0.9902011 | 1.1296357 | 0.9844189 | 1.1050618 |
| Gadd153 | 1.2487975 | 1.1220376 | 1.2835377 | 1.0411322 | 0.9968673 | 0.4850044 | 0.9611386 |
| Phase-1 RCT-109 | 1.233815 | 1.0833348 | 1.1809543 | 0.7108585 | 0.9830119 | 0.8203491 | 0.9978514 |
| Phase-1 RCT-88 | 0.7434347 | 1.7289752 | 0.8909372 | 0.8759294 | 1.2718333 | 1.1560131 | 1.1241212 |
| Ubiquitin conjugating enzyme (RAD 6 | 1.2238883 | 0.9104772 | 1.4538774 | 0.820419 | 0.7575641 | 0.858888 | 0.9641403 |
| homologue) | | | | | | | |
| Glucose-regulated protein 78 | 2.8255053 | 2.4332201 | 2.0646083 | 1.0027112 | 1.0471576 | 0.9741064 | 1.3228487 |
| Ribosomal protein L13A | 1.4832586 | 1.1533147 | 1.1264385 | 0.7074159 | 1.0623466 | 0.8928593 | 1.0944556 |
| Monocyte chemotactic protein receptor | 1.2564021 | 1.0113914 | 1.2922009 | 1.2203573 | 1.264533 | 1.2026086 | 0.7551482 |
| (CCR2) | | | | | | | |
| Fas antigen | 1.1714203 | 1.0756435 | 0.8526435 | 1.0978316 | 1.0605286 | 1.1664758 | 1.1220882 |
| Calpactin I heavy chain | 1.2417411 | 1.0758182 | 1.0192733 | 1.1700073 | 0.8662134 | 1.0368158 | 1.0832287 |
| Uncoupling protein 2 | 1.1768991 | 1.0713855 | 1.2652906 | 1.2015157 | 1.0545256 | 0.9750941 | 0.9345383 |
| Phase-1 RCT-168 | 0.9718568 | 0.8515056 | 0.8848201 | 0.6850035 | 0.909271 | 0.9892502 | 1.347934 |
| Phase-1 RCT-154 | 1.0105553 | 0.9811906 | 1.1205436 | 0.8420867 | 1.0086339 | 0.9913636 | 1.0236444 |
| Superoxide dismutase Mn | 1.468042 | 3.506713 | 0.892983 | 1.2285683 | 0.9295583 | 0.7700395 | 1.3846544 |
| Phase-1 RCT-214 | 1.4558505 | 0.9740449 | 1.0384117 | 0.9943737 | 1.137895 | 1.1530548 | 0.7116138 |
| Phase-1 RCT-225 | 1.436246 | 1.0092889 | 1.008125 | 1.0091178 | 0.9830461 | 1.0380981 | 1.0156213 |
| Phase-1 RCT-181 | 0.8511692 | 0.7412931 | 0.9097847 | 0.7813857 | 1.005116 | 0.8620614 | 0.9598826 |
| Nucleoside diphosphate kinase beta | 1.7193323 | 1.0877124 | 1.2787684 | 0.9281398 | 1.2568152 | 0.8444189 | 0.9145323 |
| isoform | | | | | | | |
| Extracellular-signal-regulated kinase 1 | 0.676679 | 0.5258663 | 0.6859908 | 0.7820998 | 0.6136556 | 0.7489668 | 1.3332881 |
| {Ribosomal protein L6} | 1.3510758 | 1.0257547 | 1.2487524 | 1.0604762 | 1.2770531 | 1.033274 | 1.2244123 |
| Aldehyde dehydrogenase, microsomal | 0.7843195 | 0.7035375 | 0.6195416 | 0.597329 | 0.9005162 | 0.7123568 | 1.4640431 |
| p53 | 1.571834 | 1.0149907 | 1.4867473 | 0.9160713 | 1.0369176 | 0.9755309 | 1.0650896 |
| Phase-1 RCT-239 | 0.8199589 | 0.8605545 | 0.7695757 | 0.9044849 | 0.8737975 | 0.864529 | 0.7724565 |
| Macrophage inflammatory protein-2 alpha | 1.7189877 | 1.0994583 | 1.8661312 | 1.7839762 | 1.1077511 | 1.1559607 | 0.7262856 |
| IgE binding protein | 1.2960093 | 1.2473719 | 0.9048504 | 1.2871315 | 1.0734574 | 1.0135958 | 1.059774 |
| Phase-1 RCT-205 | 1.4751191 | 1.0431721 | 1.7260716 | 1.1589204 | 0.9556348 | 1.066581 | 0.8361041 |
| Melanoma-associated antigen ME491 | 0.896231 | 1.0006887 | 0.9345872 | 1.0849781 | 1.1741955 | 0.9064989 | 0.8408794 |
| Phase-1 RCT-242 | 1.0475996 | 1.0915982 | 1.2243582 | 1.192073 | 1.074609 | 1.1910331 | 0.8352 |
| Enolase alpha | 2.2947683 | 0.5152126 | 1.9754124 | 0.6795118 | 0.6935996 | 0.6215389 | 1.0617068 |
| Thymosin beta-10 | 1.1974297 | 0.8963423 | 1.128062 | 0.9615769 | 1.0056243 | 0.8072347 | 1.0193958 |
| Cofilin | 0.8394011 | 0.6409223 | 0.6461364 | 0.9403794 | 0.906169 | 0.9861858 | 1.159961 |
| Carbonyl reductase | 1.1105323 | 1.0769106 | 1.1485449 | 1.0026492 | 1.0103858 | 1.1113406 | 0.8745745 |
| MAP kinase kinase | 1.3811922 | 0.9661044 | 1.5602373 | 0.9575723 | 0.8280167 | 0.8945473 | 0.9504942 |
| Phase-1 RCT-162 | 0.9921196 | 1.1239445 | 1.0492289 | 1.0054308 | 1.2697009 | 1.1342592 | 1.0508149 |
| Phase-1 RCT-72 | 0.9880831 | 1.1420139 | 0.9626055 | 0.9946396 | 1.0347376 | 0.9567167 | 0.8382592 |
| Ribosomal protein S8 | 1.3840513 | 1.2027864 | 1.3188925 | 0.863822 | 1.6345536 | 1.0348893 | 1.3208598 |
| Proliferating cell nuclear antigen gene | 0.9813408 | 1.0749782 | 1.0816242 | 1.1710718 | 1.0952609 | 1.2903746 | 0.9085429 |
| Phase-1 RCT-192 | 2.2982483 | 0.9227464 | 1.241424 | 0.8963103 | 1.1052252 | 1.0898551 | 1.0895913 |
| IkB-a | 1.487841 | 0.5362631 | 1.1714851 | 0.7433963 | 0.7902769 | 0.697037 | 0.9947685 |
| Endogenous retroviral sequence, 5′ and | 2.7517867 | 1.2329273 | 1.2969363 | 0.9325558 | 0.8867677 | 0.9106964 | 0.7283608 |
| 3′ LTR | | | | | | | |
| Phase-1 RCT-37 | 1.0543014 | 1.1224242 | 1.0788693 | 1.1564903 | 1.1590062 | 1.1071378 | 0.9312566 |
| Matrix metalloproteinase-1 | 1.1946476 | 0.9059116 | 0.9746371 | 1.2955819 | 0.9946038 | 1.108152 | 1.0612762 |
| Cyclin G | 0.7755783 | 1.1054544 | 0.8352638 | 1.1674308 | 1.0757862 | 1.1428696 | 0.9706492 |
| DNA polymerase beta | 1.3270873 | 0.97 | 1.1967757 | 0.9131655 | 0.8849738 | 0.9229431 | 1.2002832 |
| Phase-1 RCT-127 | 0.9797789 | 1.0205814 | 1.0630614 | 1.1461736 | 1.0588452 | 1.2058762 | 0.8578207 |
| Multidrug resistant protein-2 | 0.7485015 | 1.0400575 | 1.3277781 | 0.9303657 | 1.0131334 | 0.8597302 | 0.8270024 |
| Ribosomal protein S9 | 1.3249532 | 0.9872906 | 1.5865046 | 1.037402 | 0.8036239 | 0.8766888 | 0.9671615 |
| Phase-1 RCT-12 | 1.4580699 | 0.258869 | 1.0531036 | 1.0736146 | 0.8411411 | 1.0268844 | 1.1258504 |
| Beta-tubulin, class I | 1.9180927 | 1.1745454 | 1.0745449 | 1.0179892 | 0.7676859 | 0.9845173 | 1.1768668 |
| Argininosuccinate lyase | 0.831472 | 0.6031745 | 0.5637889 | 0.6016048 | 0.7109531 | 0.599299 | 1.2292593 |
| Elongation factor-1 alpha | 1.0820181 | 0.7111693 | 0.8009331 | 0.5896128 | 0.9061195 | 0.7721649 | 1.2716962 |
| Phase-1 RCT-55 | 1.2196379 | 1.3101298 | 1.1220676 | 1.15589 | 1.1424786 | 1.0974236 | 0.6802774 |
| Macrophage inflammatory protein-1 alpha | 1.0126565 | 1.056427 | 1.4079093 | 1.2096477 | 1.2964143 | 1.2204634 | 0.7845062 |
| c-H-ras | 0.7950777 | 0.9876363 | 0.8454319 | 0.8813387 | 0.7116705 | 0.8005546 | 1.0926784 |
| Multidrug resistant protein-1 | 0.8298513 | 1.0966611 | 0.7939226 | 1.0598531 | 1.018352 | 1.0804597 | 0.8345949 |
| T-cell cyclophilin | 1.4727225 | 0.8791379 | 1.1480366 | 0.7336664 | 1.2204411 | 0.8325869 | 1.3281887 |
| Phase-1 RCT-185 | 0.9276577 | 0.5706543 | 0.7474245 | 0.5641115 | 0.8436048 | 0.5885812 | 1.1367952 |
| 25-DX | 0.9760989 | 0.9603667 | 0.873754 | 0.8933885 | 0.9033963 | 0.8567354 | 1.216698 |
| Ref-1 | 1.1939623 | 1.3006818 | 1.0947155 | 1.0015463 | 0.8522387 | 0.9434549 | 1.0021678 |
| Apolipoprotein CIII | 0.7388774 | 0.5098157 | 0.694974 | 0.4364303 | 0.6350741 | 0.5293385 | 1.2366505 |
| NADPH quinone oxidoreductase-1 (DT- | 1.2342807 | 1.1618181 | 0.9982382 | 1.0403365 | 0.8216108 | 0.7630644 | 1.5516554 |
| diaphorase) | | | | | | | |
| Pyruvate kinase, muscle | 1.0070374 | 1.1437368 | 0.9443246 | 1.3722893 | 1.1649499 | 1.1174672 | 0.8888143 |
| | THEO | THEO | THEO | THEO | THEO | ANIT | ANIT |
| Compound-Dose (2) | 25 | 25 | 100 | 100 | 100 | 60 | 60 |
| Animal Number (3) | 2525 | 2526 | 2534 | 2535 | 2536 | 1654 | 1655 |
| Liver Toxicity Necrosis Classification (4) | no | no | no | no | no | yes | yes |
| Gene Name (5) | | | | | | | |
| Phase-1 RCT-78 | 1.1220393 | 1.1266272 | 0.9388853 | 0.9198881 | 1.0755862 | 0.6961321 | 0.8470088 |
| Gamma-actin, cytoplasmic | 0.9434511 | 0.7417277 | 0.7858459 | 0.9789131 | 0.7294344 | 2.8152604 | 2.7905335 |
| Matrin F/G | 1.07155 | 0.9838756 | 0.8634632 | 0.7548612 | 0.8688873 | 0.6892105 | 0.5658238 |
| Zinc finger protein | 0.8035369 | 0.9846456 | 0.919289 | 0.9311262 | 1.0060853 | 2.4493477 | 2.337706 |
| Cathepsin L, sequence 2 | 0.9381852 | 1.0867897 | 0.9784191 | 0.7744115 | 0.8270517 | 1.7213274 | 1.6381568 |
| Gadd45 | 1.1350304 | 1.0493032 | 1.003104 | 0.999494 | 0.8582763 | 2.088889 | 1.8500834 |
| Phase-1 RCT-144 | 1.0173373 | 0.9628124 | 1.051437 | 0.9582852 | 0.9664823 | 1.2269295 | 1.2501448 |
| Phase-1 RCT-145 | 0.9074849 | 1.0265571 | 0.9157419 | 0.9676859 | 0.9506524 | 1.4177402 | 1.32557 |
| Phase-1 RCT-50 | 0.8941805 | 0.9763002 | 0.9824991 | 1.0060899 | 0.9838153 | 1.5586743 | 2.7248285 |
| Phase-1 RCT-92 | 1.1729012 | 1.20954 | 0.9068178 | 0.9460542 | 0.8734289 | 0.4691075 | 0.3674097 |
| Phase-1 RCT-89 | 0.9263505 | 1.027861 | 0.7177791 | 0.6244379 | 0.7236195 | 0.6430527 | 0.6246479 |
| 14-3-3 zeta | 0.9403356 | 1.1047875 | 0.9074978 | 0.8827285 | 1.0356712 | 1.565919 | 1.8138217 |
| Dynamin-1 (D100) | 1.0644503 | 1.0897633 | 1.0086985 | 0.7873054 | 1.0084964 | 0.5147045 | 0.5758659 |
| Insulin-like growth factor binding protein 1 | 1.0404092 | 1.1234801 | 0.9599903 | 0.8370088 | 0.946133 | 1.8059268 | 2.2281926 |
| L-gulono-gamma-lactone oxidase | 1.2701422 | 1.6550367 | 0.8044473 | 0.9402264 | 0.7377338 | 0.3179815 | 0.3028709 |
| Ornithine decarboxylase | 1.1186783 | 1.1695346 | 1.1164699 | 1.2337508 | 1.000994 | 1.9478767 | 1.5830907 |
| PAR interacting protein | 0.9138687 | 1.0098084 | 0.839736 | 0.9431425 | 0.9076476 | 1.3840002 | 1.2157574 |
| Phase-1 RCT-128 | 1.2484956 | 1.1414629 | 0.6498232 | 0.727314 | 0.8531917 | 0.4078431 | 0.4524199 |
| Phase-1 RCT-180 | 1.1747284 | 0.8950982 | 0.8893652 | 0.9490431 | 0.8475983 | 1.4825579 | 1.1481745 |
| Phase-1 RCT-182 | 1.3786206 | 1.1622378 | 0.8071315 | 0.8231455 | 0.9101691 | 0.5521258 | 0.6272612 |
| Phase-1 RCT-207 | 0.711282 | 0.8034921 | 0.8852928 | 0.8652619 | 0.8805918 | 1.681586 | 1.3995681 |
| Phase-1 RCT-213 | 0.9400621 | 0.949598 | 1.2005879 | 1.2547728 | 0.9709979 | 1.50194 | 1.2385921 |
| Phase-1 RCT-256 | 1.1390094 | 1.2869605 | 0.8932656 | 0.9375385 | 0.7729853 | 0.463885 | 0.3605489 |
| Phase-1 RCT-258 | 0.8752708 | 0.9701706 | 0.8993474 | 0.9289058 | 0.9313084 | 1.2287164 | 1.123066 |
| Phase-1 RCT-264 | 1.2366326 | 0.8034787 | 0.6617537 | 0.6007329 | 0.840636 | 0.3275119 | 0.4368199 |
| Phase-1 RCT-271 | 1.2063156 | 1.3496933 | 0.7494949 | 0.8043286 | 0.7482653 | 0.6102762 | 0.5468586 |
| Phase-1 RCT-288 | 0.7410417 | 0.8887366 | 0.9087445 | 0.9400867 | 1.0838721 | 0.4036785 | 0.4882846 |
| Phase-1 RCT-38 | 1.1663319 | 1.3534489 | 0.9491294 | 0.9915039 | 0.8093461 | 0.4384516 | 0.3338689 |
| Phase-1 RCT-39 | 0.935647 | 0.8952121 | 1.039871 | 1.0944964 | 1.120013 | 1.394147 | 1.7354772 |
| Phase-1 RCT-68 | 0.994614 | 1.0740385 | 1.0246825 | 1.2335961 | 0.9391443 | 1.2066668 | 1.077988 |
| Phase-1 RCT-33 | 1.1922166 | 1.4344286 | 0.911099 | 0.8080772 | 1.0134815 | 0.5376451 | 0.4719624 |
| Phase-1 RCT-36 | 1.0061804 | 1.1493559 | 1.0471901 | 0.9124379 | 1.1267406 | 0.7296611 | 0.7008768 |
| 17-beta hydroxysteroid dehydrogenase, | 1.7251128 | 3.7807474 | 0.8511449 | 0.8947511 | 1.3390231 | 0.1813125 | 0.3131616 |
| type 2 | | | | | | | |
| Senescence marker protein-30 | 1.2577316 | 1.0616696 | 1.0748152 | 1.0400493 | 1.0165031 | 0.2551819 | 0.5259507 |
| Ribosomal protein S17 | 1.0330361 | 1.177434 | 1.3389037 | 1.291196 | 0.9394962 | 1.0568964 | 0.866909 |
| Phase-1 RCT-83 | 0.9530403 | 0.8970445 | 0.9896238 | 1.0515077 | 1.129635 | 0.4109602 | 0.6470055 |
| Phase-1 RCT-49 | 0.8958899 | 0.9717873 | 0.8392482 | 0.8415858 | 0.8674936 | 0.9724519 | 1.035773 |
| Phase-1 RCT-48 | 1.3074293 | 0.9902011 | 0.8421724 | 0.7051029 | 1.0403283 | 0.6044615 | 0.7096151 |
| Phase-1 RCT-40 | 1.0547429 | 0.9915972 | 0.7804128 | 0.860847 | 0.9638659 | 0.5076305 | 0.7767282 |
| Phase-1 RCT-296 | 1.1304826 | 1.2382525 | 0.408118 | 0.3549582 | 0.6180584 | 0.1922587 | 0.2773164 |
| Phase-1 RCT-295 | 1.1593333 | 1.3842819 | 1.0933318 | 1.173226 | 0.9629908 | 0.8879778 | 0.8172959 |
| Phase-1 RCT-291 | 1.1348339 | 1.1891232 | 0.9054197 | 0.8424065 | 0.9649891 | 0.4497288 | 0.557257 |
| Phase-1 RCT-270 | 1.0547317 | 0.7465306 | 1.0386169 | 0.9197203 | 0.8572939 | 0.4151418 | 0.3587099 |
| Phase-1 RCT-241 | 0.7021579 | 0.9019584 | 1.1394907 | 1.0942625 | 1.1416953 | 2.4354577 | 2.2945616 |
| Phase-1 RCT-191 | 1.0365446 | 1.1705621 | 0.9201004 | 1.083967 | 1.0494838 | 1.6250434 | 1.39287 |
| Phase-1 RCT-189 | 1.2188154 | 0.9831485 | 0.932184 | 0.952063 | 0.9467708 | 0.6380764 | 0.8485993 |
| Phase-1 RCT-179 | 0.9466672 | 0.9669802 | 0.8466792 | 0.7849215 | 1.0230837 | 1.1305246 | 0.9690059 |
| Phase-1 RCT-152 | 1.083962 | 1.1544788 | 1.0012681 | 0.9123893 | 0.8629348 | 1.2765598 | 1.298122 |
| Phase-1 RCT-123 | 0.9996912 | 1.0542737 | 1.0088018 | 1.1000786 | 1.036045 | 0.8956644 | 0.902762 |
| Paraoxonase 1 | 1.117527 | 1.0779529 | 0.6251723 | 0.5174809 | 0.777698 | 0.4711399 | 0.5081691 |
| Organic anion transporter 3 | 0.9792429 | 0.8425502 | 1.0918518 | 1.8182615 | 1.1941669 | 0.759185 | 0.6428167 |
| N-hydroxy-2-acetylaminofluorene | 1.2734567 | 1.3237303 | 0.8021085 | 0.6011469 | 1.114861 | 0.2021088 | 0.5063289 |
| sulfotransferase (ST1C1) | | | | | | | |
| Integrin beta1 | 0.8697059 | 0.9569098 | 0.9858753 | 1.0217527 | 1.0190204 | 1.7162081 | 2.1971838 |
| Insulin-like growth factor binding protein 3 | 1.1590829 | 0.8750337 | 0.83332 | 0.6739986 | 0.8327453 | 0.6269974 | 0.6299705 |
| ID-1 | 0.7902851 | 0.9996827 | 1.27314 | 1.1379935 | 1.0786939 | 1.8451518 | 1.6511902 |
| Hepatic lipase | 1.113921 | 0.8597364 | 0.6769535 | 0.4063112 | 1.0597035 | 0.4280441 | 0.5238654 |
| Heme oxygenase | 0.9111992 | 1.0706968 | 1.0146351 | 0.7345184 | 0.8563094 | 1.479942 | 2.1035345 |
| Equilbrative nitrobenzylthioinosine- | 1.2549496 | 1.1335163 | 1.1670045 | 1.1187329 | 0.9149723 | 0.350353 | 0.528926 |
| sensitive nucleoside transporter | | | | | | | |
| Epidermal growth factor | 0.9791992 | 1.1796695 | 0.9234029 | 0.8602191 | 0.849893 | 0.8731699 | 0.8649455 |
| c-myc | 0.948236 | 2.1428418 | 0.8582542 | 0.7990935 | 0.8681663 | 2.6966634 | 1.8456204 |
| Carbonic anhydrase III | 1.5974911 | 1.1194391 | 0.4051793 | 0.6725298 | 0.736232 | 0.0873532 | 0.2614988 |
| Beta-actin | 1.1465247 | 0.8235149 | 0.5805013 | 0.7854807 | 0.6145368 | 1.3863703 | 1.3947511 |
| Bax (alpha) | 0.9178671 | 0.9759055 | 1.0446929 | 1.1944269 | 1.093296 | 1.3595716 | 1.5977923 |
| Alpha 1 - inhibitor III | 1.0218555 | 0.6981795 | 0.5702699 | 0.3634046 | 0.9085642 | 0.4167133 | 0.3635373 |
| 60S ribosomal protein L6 | 1.0325091 | 1.0120535 | 1.0916188 | 1.0156378 | 0.8335154 | 1.3241475 | 1.1786474 |
| Phase-1 RCT-117 | 0.9552888 | 0.6062916 | 0.9645835 | 0.9277731 | 0.8835928 | 0.7400679 | 0.7878104 |
| Phase-1 RCT-102 | 0.8436851 | 1.2655032 | 0.8194567 | 0.6615052 | 1.0957991 | 0.3032405 | 0.4666673 |
| 3-hydroxyisobutyrate dehydrogenase | 1.2434905 | 1.158142 | 0.8928415 | 0.7976742 | 0.904689 | 0.4631926 | 0.5492526 |
| ATPase inhibitor (rat mitochondrial IF1 | 1.2819902 | 1.0638522 | 0.8367152 | 0.5344215 | 0.7873751 | 0.4249822 | 0.5269502 |
| protein) | | | | | | | |
| Alpha-2-macroglobulin | 0.7587047 | 0.9390821 | 34.372776 | 26.288652 | 15.052653 | 9.551549 | 2.6563401 |
| Phase-1 RCT-137 | 1.2581524 | 1.0157923 | 0.6091464 | 0.6698488 | 0.9164694 | 0.5393679 | 0.4999779 |
| Phase-1 RCT 252 | 1.3389666 | 1.210047 | 0.5898305 | 0.6499562 | 0.7342437 | 0.5730206 | 0.3896135 |
| Phase-1 RCT-65 | 1.1964607 | 1.4172748 | 1.3983415 | 1.2834874 | 1.1699543 | 1.3543745 | 1.6250447 |
| Liver fatty acid binding protein | 1.0051203 | 0.7508866 | 0.7552847 | 0.7554367 | 0.767019 | 0.4865508 | 0.4626451 |
| Carbamyl phosphate synthetase I | 1.4237497 | 1.276245 | 0.5902479 | 0.6988373 | 0.7694767 | 0.5301982 | 0.3750241 |
| c-jun | 0.958294 | 1.3470756 | 0.8807218 | 0.8821594 | 1.0948552 | 4.1824727 | 6.5412016 |
| Transthyretin | 1.2006359 | 0.7901817 | 0.6691372 | 0.6464093 | 0.8291809 | 0.2605753 | 0.3533776 |
| Aflatoxin B1 aldehyde reductase | 0.8987738 | 0.8878503 | 0.5200019 | 0.4532877 | 0.4891462 | 0.8624408 | 0.5412217 |
| Phase-1 RCT-15 | 1.2945489 | 0.9845755 | 1.1453357 | 1.0817924 | 1.0770075 | 1.3116584 | 1.2204317 |
| Insulin-like growth factor I | 1.0739185 | 0.9020818 | 0.8061391 | 0.4769562 | 0.8292748 | 0.3523279 | 0.5472131 |
| Sodium/bile acid cotransporter | 1.1232232 | 1.1564685 | 0.616581 | 0.5708982 | 0.8713076 | 0.6021556 | 0.5058445 |
| Organic cation transporter 3 | 0.9882796 | 0.9907386 | 1.0858935 | 1.0141077 | 0.9312543 | 1.3428649 | 1.1836398 |
| Gadd153 | 0.9195063 | 0.9959227 | 1.1672707 | 1.1495482 | 0.9845557 | 0.9947393 | 1.3535042 |
| Phase-1 RCT-109 | 0.8904433 | 1.0015383 | 0.9676464 | 0.9998475 | 0.8106324 | 1.1541326 | 0.9714038 |
| Phase-1 RCT-88 | 1.0079993 | 1.0481205 | 0.9663817 | 1.1775594 | 1.2058598 | 0.7571328 | 0.7798724 |
| Ubiquitin conjugating enzyme (RAD 6 | 0.8951305 | 1.0422952 | 0.8081126 | 0.9006992 | 0.8135094 | 1.2910385 | 0.9672893 |
| homologue) | | | | | | | |
| Glucose-regulated protein 78 | 1.0920377 | 0.9706768 | 1.5877883 | 1.1940827 | 0.9887945 | 1.2423178 | 1.4959893 |
| Ribosomal protein L13A | 0.9489824 | 1.0436583 | 0.999066 | 1.0085459 | 0.8296368 | 1.2558315 | 0.9857935 |
| Monocyte chemotactic protein receptor | 0.7507481 | 0.8669027 | 1.0708545 | 1.1765018 | 1.1772205 | 1.3120111 | 1.2535402 |
| (CCR2) | | | | | | | |
| Fas antigen | 0.861832 | 1.0287813 | 1.1768016 | 1.1950091 | 1.1224891 | 1.2441623 | 1.2612631 |
| Calpactin I heavy chain | 0.9643956 | 0.9399945 | 1.0842972 | 1.1248325 | 0.9388027 | 1.0371984 | 1.2169139 |
| Uncoupling protein 2 | 0.8753982 | 0.9261144 | 0.8550376 | 0.9084612 | 0.80994 | 1.0997783 | 1.099583 |
| Phase-1 RCT-168 | 1.2113774 | 1.4237919 | 1.3341461 | 1.1300302 | 1.2070315 | 0.8385453 | 0.9500386 |
| Phase-1 RCT-154 | 0.9260086 | 1.0449244 | 1.0695142 | 1.2774626 | 0.94463 | 1.0681405 | 1.0714875 |
| Superoxide dismutase Mn | 1.0474403 | 1.0644368 | 1.209133 | 1.5191853 | 0.9384253 | 2.3443198 | 1.9280921 |
| Phase-1 RCT-214 | 0.7978216 | 0.9310579 | 0.9902341 | 0.9880864 | 1.111068 | 0.6796362 | 0.7879437 |
| Phase-1 RCT-225 | 0.8648955 | 0.8610451 | 0.5451729 | 1.520862 | 0.7126507 | 1.028194 | 1.7131829 |
| Phase-1 RCT-181 | 1.0731744 | 1.0237112 | 1.1418462 | 1.0818194 | 0.8648255 | 0.8645125 | 0.8001847 |
| Nucleoside diphosphate kinase beta | 0.8761722 | 1.0204362 | 1.1135342 | 1.1775426 | 1.054133 | 1.0977099 | 1.248496 |
| isoform | | | | | | | |
| Extracellular-signal-regulated kinase 1 | 1.2487929 | 1.0538281 | 0.5114485 | 0.7659125 | 0.7908775 | 0.1192284 | 0.2877547 |
| {Ribosomal protein L6} | 1.0639503 | 1.0688848 | 1.1064535 | 1.0005352 | 0.8164751 | 1.2078929 | 1.0232292 |
| Aldehyde dehydrogenase, microsomal | 1.2596482 | 1.0506144 | 0.9396829 | 0.894696 | 0.9189517 | 0.9066032 | 0.603242 |
| p53 | 1.0654161 | 1.101488 | 0.8543395 | 0.9285175 | 0.8019966 | 1.3007476 | 1.3982984 |
| Phase-1 RCT-239 | 0.8540638 | 0.8836891 | 0.7905749 | 0.8914962 | 0.9475409 | 0.9437185 | 0.7811502 |
| Macrophage inflammatory protein-2 alpha | 0.7968088 | 0.9607267 | 1.0842766 | 1.118871 | 1.179175 | 2.5375724 | 2.5458276 |
| IgE binding protein | 0.9526317 | 0.9266123 | 1.0498761 | 1.0793182 | 0.9424196 | 1.1841584 | 1.1343348 |
| Phase-1 RCT-205 | 1.0685853 | 1.0566351 | 0.9854053 | 0.9956825 | 0.8641006 | 1.500363 | 1.4831738 |
| Melanoma-associated antigen ME491 | 0.9424139 | 0.9342061 | 1.3887467 | 1.7298536 | 0.8946096 | 1.126574 | 1.0606791 |
| Phase-1 RCT-242 | 0.8527405 | 0.9586331 | 0.9974814 | 1.05202 | 1.0731747 | 2.1078625 | 3.7647343 |
| Enolase alpha | 1.2251207 | 1.1664431 | 1.0463818 | 1.0029914 | 0.791374 | 1.2667087 | 0.8769494 |
| Thymosin beta-10 | 0.9292551 | 0.9739209 | 0.9707944 | 1.02908 | 0.7933047 | 1.2212476 | 1.057961 |
| Cofilin | 1.180843 | 0.9677522 | 1.1296276 | 0.8985124 | 0.9337457 | 1.1345875 | 0.9967017 |
| Carbonyl reductase | 0.9226413 | 1.0041983 | 1.1043767 | 1.1847073 | 1.1232936 | 1.3311648 | 1.2950934 |
| MAP kinase kinase | 0.861832 | 0.9337852 | 1.0489523 | 1.076368 | 1.0251946 | 1.0744746 | 1.1940694 |
| Phase-1 RCT-162 | 1.0912927 | 1.041339 | 1.174185 | 1.0426799 | 0.9750128 | 1.0252061 | 1.0628316 |
| Phase-1 RCT-72 | 0.9405845 | 0.9267927 | 0.9396274 | 0.9796588 | 1.009357 | 1.0519296 | 1.2393016 |
| Ribosomal protein S8 | 1.147658 | 1.21466 | 1.2606599 | 1.2228048 | 0.9492608 | 1.0948827 | 0.8589276 |
| Proliferating cell nuclear antigen gene | 1.0566753 | 1.0712969 | 0.8844885 | 0.8340799 | 0.9471925 | 1.2120155 | 1.1854339 |
| Phase-1 RCT-192 | 0.8910543 | 1.2469215 | 0.8987751 | 1.0454226 | 1.2293906 | 1.1743883 | 0.9599591 |
| IkB-a | 1.1736759 | 1.1615682 | 1.0670236 | 0.9245093 | 0.8838147 | 1.2784944 | 0.9227232 |
| Endogenous retroviral sequence, 5′ and | 0.8801769 | 1.0072074 | 0.9441097 | 1.1292355 | 0.9172252 | 0.8816609 | 1.0033368 |
| 3′ LTR | | | | | | | |
| Phase-1 RCT-37 | 1.1018976 | 1.0684204 | 1.0606079 | 1.0345812 | 0.9012753 | 1.0136738 | 0.9576539 |
| Matrix metalloproteinase-1 | 1.2382965 | 1.0466099 | 1.0554758 | 1.0649526 | 0.9057994 | 0.7767853 | 0.6894357 |
| Cyclin G | 0.9825627 | 1.214957 | 1.5162739 | 1.4245207 | 1.1122184 | 1.4286355 | 1.6611838 |
| DNA polymerase beta | 1.0947127 | 1.1401175 | 1.1456436 | 1.1414534 | 1.0558777 | 1.0203735 | 0.8846288 |
| Phase-1 RCT-127 | 0.8733294 | 0.9557945 | 0.8780219 | 0.9217835 | 0.9253096 | 0.9599234 | 1.0055574 |
| Multidrug resistant protein-2 | 0.8308448 | 1.0227686 | 1.0233074 | 0.8894162 | 0.8243977 | 4.008009 | 3.4536848 |
| Ribosomal protein S9 | 0.965985 | 1.131555 | 0.7332107 | 0.8478302 | 0.7787006 | 1.3421649 | 0.8415982 |
| Phase-1 RCT-12 | 0.9229129 | 1.0069523 | 1.1085943 | 1.0541285 | 1.0822709 | 1.0967145 | 1.2509782 |
| Beta-tubulin, class I | 0.9050277 | 1.01372 | 1.209421 | 1.0307627 | 1.0623822 | 1.2549764 | 1.4095595 |
| Argininosuccinate lyase | 1.3823186 | 1.2346812 | 0.8309221 | 0.802727 | 0.8253072 | 1.4018197 | 1.0604713 |
| Elongation factor-1 alpha | 0.8325455 | 1.1190877 | 0.8460594 | 0.9092454 | 0.7288212 | 1.0457118 | 0.8409534 |
| Phase-1 RCT-55 | 0.9093583 | 0.7852604 | 0.9818007 | 1.0473076 | 1.0428748 | 1.8226064 | 2.1779242 |
| Macrophage inflammatory protein-1 alpha | 0.7368042 | 0.7790265 | 0.9600059 | 1.1748543 | 1.1202792 | 1.1797495 | 1.5470227 |
| c-H-ras | 0.9631505 | 0.9128817 | 1.1778939 | 1.2190326 | 0.9354211 | 1.5805224 | 1.3348665 |
| Multidrug resistant protein-1 | 0.8246425 | 1.0120523 | 0.8802699 | 0.9307888 | 0.8466981 | 3.6975641 | 3.1814823 |
| T-cell cyclophilin | 1.1729101 | 1.118609 | 1.127306 | 1.0891328 | 0.9434121 | 0.9962068 | 0.830153 |
| Phase-1 RCT-185 | 1.3528298 | 1.1403501 | 0.6409537 | 0.5706481 | 0.8352439 | 0.566964 | 0.6588851 |
| 25-DX | 1.1110549 | 1.2172984 | 0.9666016 | 0.8664042 | 0.9054251 | 0.9367636 | 0.8444185 |
| Ref-1 | 1.0774491 | 1.0750184 | 1.0495092 | 1.0560819 | 1.0487334 | 1.237666 | 1.2532275 |
| Apolipoprotein CIII | 1.0614648 | 0.9633781 | 1.1462439 | 1.0424675 | 0.9057154 | 0.8120633 | 0.712142 |
| NADPH quinone oxidoreductase-1 (DT- | 1.1967379 | 0.8914231 | 1.7038383 | 0.8988265 | 1.4750458 | 1.5951164 | 1.27731 |
| diaphorase) | | | | | | | |
| Pyruvate kinase, muscle | 0.9266922 | 1.0412067 | 0.8767597 | 0.9030135 | 0.9170635 | 1.5084109 | 1.4707555 |
| | ANIT | APAP | APAP | APAP | BRB | BRB | BRB |
| Compound-Dose (2) | 60 | 1000 | 1000 | 1000 | 200 | 200 | 200 |
| Animal Number (3) | 1656 | 2134 | 2135 | 2136 | 2346 | 2347 | 2348 |
| Liver Toxicity Necrosis Classification (4) | yes | yes | yes | yes | yes | yes | yes |
| Gene Name (5) | | | | | | | |
| Phase-1 RCT-78 | 0.7892923 | 0.7808704 | 0.6313698 | 0.4383601 | 0.63516 | 0.7712065 | 0.5321541 |
| Gamma-actin, cytoplasmic | 2.783659 | 1.8582921 | 1.5787565 | 10.269378 | 6.3413463 | 2.562591 | 6.8288484 |
| Matrin F/G | 0.5744017 | 1.1557255 | 0.8929568 | 0.5379188 | 0.65456 | 0.8986663 | 0.4634787 |
| Zinc finger protein | 2.670814 | 0.9303169 | 0.8117558 | 1.7251326 | 2.7530024 | 0.8659638 | 5.729396 |
| Cathepsin L, sequence 2 | 2.283068 | 1.074807 | 1.338568 | 4.1737027 | 2.8960137 | 1.5487506 | 2.3439903 |
| Gadd45 | 1.8602532 | 0.8220603 | 1.3169364 | 2.7411542 | 3.9154673 | 0.942708 | 3.2177715 |
| Phase-1 RCT-144 | 1.1867278 | 0.9816055 | 1.1773083 | 2.422509 | 3.0254192 | 0.9767266 | 3.379373 |
| Phase-1 RCT-145 | 1.373649 | 1.2001864 | 1.5215938 | 2.3350213 | 1.6164194 | 1.1105763 | 2.2340593 |
| Phase-1 RCT-50 | 2.7353585 | 1.054244 | 1.0322771 | 2.637211 | 1.8690739 | 0.8449889 | 3.9277573 |
| Phase-1 RCT-92 | 0.5628777 | 0.6478241 | 0.6327204 | 0.3995366 | 0.5270688 | 1.0341121 | 0.4897167 |
| Phase-1 RCT-89 | 0.7949664 | 0.8349818 | 0.676441 | 0.3345693 | 0.4414944 | 1.1624697 | 0.4138589 |
| 14-3-3 zeta | 1.4153996 | 1.27887 | 1.3397921 | 2.1931944 | 1.4373559 | 1.2539016 | 1.5923091 |
| Dynamin-1 (D100) | 0.467532 | 0.8585791 | 0.7809165 | 0.4206284 | 0.8053063 | 0.7635024 | 0.8226298 |
| Insulin-like growth factor binding protein 1 | 2.4912002 | 1.5541166 | 1.8837394 | 3.7856872 | 6.49358 | 1.353118 | 3.955229 |
| L-gulono-gamma-lactone oxidase | 0.4752978 | 0.5988902 | 0.4215989 | 0.1969441 | 0.2873403 | 0.7202513 | 0.2090522 |
| Ornithine decarboxylase | 1.8256718 | 1.9183862 | 2.1628938 | 3.0924182 | 2.7621546 | 1.4877541 | 2.287528 |
| PAR interacting protein | 1.1198193 | 1.1032678 | 1.4794549 | 2.2465057 | 2.294099 | 1.2070475 | 2.9862342 |
| Phase-1 RCT-128 | 0.3382263 | 0.7458431 | 0.684723 | 0.2366052 | 0.8250362 | 1.3068181 | 0.5385185 |
| Phase-1 RCT-180 | 1.5800836 | 1.3623645 | 1.5232956 | 1.8385341 | 1.2648929 | 1.2552742 | 1.5028497 |
| Phase-1 RCT-182 | 0.5579845 | 0.6489257 | 0.5529457 | 0.3135115 | 1.3480315 | 1.3008316 | 0.6879845 |
| Phase-1 RCT-207 | 1.3459651 | 1.2898468 | 1.3983328 | 2.7147381 | 2.037197 | 1.0707659 | 3.252716 |
| Phase-1 RCT-213 | 1.2331116 | 1.3166662 | 1.6070114 | 2.1341743 | 1.467437 | 1.0596248 | 1.3876842 |
| Phase-1 RCT-256 | 0.5427848 | 0.7770574 | 0.6184471 | 0.3034725 | 0.3659292 | 1.1538446 | 0.3281332 |
| Phase-1 RCT-258 | 1.138353 | 1.1984203 | 1.2893456 | 1.6283574 | 1.1859962 | 0.9631207 | 1.5418757 |
| Phase-1 RCT-264 | 0.5232891 | 0.4973377 | 0.4838935 | 0.3685766 | 0.8030628 | 1.0725478 | 0.4349538 |
| Phase-1 RCT-271 | 0.4629554 | 0.7960979 | 0.8118808 | 0.5952261 | 0.5824906 | 0.9650761 | 0.5278825 |
| Phase-1 RCT-288 | 0.4574211 | 0.7642786 | 0.6193053 | 0.2497753 | 0.3955931 | 1.013478 | 0.3601181 |
| Phase-1 RCT-38 | 0.5591697 | 0.8079268 | 0.6658584 | 0.4284167 | 0.3181279 | 1.105129 | 0.2601448 |
| Phase-1 RCT-39 | 1.2926806 | 1.0720022 | 1.1113166 | 1.5111115 | 1.2794008 | 0.9004853 | 2.1609643 |
| Phase-1 RCT-68 | 1.2159271 | 1.3829609 | 1.4294881 | 2.0063844 | 1.9724381 | 1.1215724 | 1.8874464 |
| Phase-1 RCT-33 | 0.5616747 | 0.8252382 | 0.7278087 | 0.486732 | 0.4270199 | 0.9747322 | 0.4560216 |
| Phase-1 RCT-36 | 0.772467 | 0.9342113 | 0.8311461 | 0.7084122 | 0.7232555 | 0.7081659 | 0.8302805 |
| 17-beta hydroxysteroid dehydrogenase, | 0.3821037 | 0.4475642 | 0.3153198 | 0.2024103 | 0.4318697 | 0.6348622 | 0.2940416 |
| type 2 | | | | | | | |
| Senescence marker protein-30 | 0.4065626 | 0.4099447 | 0.2444617 | 0.0982913 | 0.236371 | 0.8944992 | 0.1786024 |
| Ribosomal protein S17 | 1.0576639 | 1.2298427 | 2.0905206 | 2.4652085 | 2.853082 | 2.060768 | 2.4397662 |
| Phase-1 RCT-83 | 0.4189972 | 0.6151087 | 0.4372851 | 0.3262761 | 0.6203949 | 0.7290333 | 0.5094547 |
| Phase-1 RCT-49 | 1 | 0.9421995 | 1.094226 | 2.166884 | 1.5863192 | 0.7475788 | 2.3540325 |
| Phase-1 RCT-48 | 0.6140745 | 1.1263044 | 0.6761991 | 0.5881342 | 0.6911385 | 1.1764657 | 0.5359991 |
| Phase-1 RCT-40 | 0.5956445 | 0.7273161 | 0.612156 | 0.3765631 | 0.5964158 | 1.1619401 | 0.5296916 |
| Phase-1 RCT-296 | 0.2491641 | 0.4087412 | 0.2214505 | 0.3469629 | 0.3367844 | 0.689222 | 0.2707186 |
| Phase-1 RCT-295 | 0.9300347 | 1.4092424 | 1.5863334 | 1.9928924 | 2.141201 | 1.9446859 | 1.9158632 |
| Phase-1 RCT-291 | 0.4965149 | 0.8483654 | 0.6018884 | 0.3593634 | 0.8945057 | 1.1988659 | 0.6860547 |
| Phase-1 RCT-270 | 0.3633713 | 0.7857366 | 0.5636744 | 0.3204545 | 0.5515848 | 1.298134 | 0.4056625 |
| Phase-1 RCT-241 | 1.9338969 | 1.1525435 | 1.0752367 | 2.3062935 | 2.0369186 | 0.8758043 | 2.5844228 |
| Phase-1 RCT-191 | 1.5593768 | 1.4874156 | 1.4979564 | 2.5144691 | 1.728489 | 1.301535 | 2.139248 |
| Phase-1 RCT-189 | 0.6801832 | 0.7548308 | 0.6613784 | 0.3340811 | 0.5887001 | 0.97497 | 0.5519788 |
| Phase-1 RCT-179 | 1.0977058 | 1.0768157 | 1.3764162 | 2.4592183 | 2.977741 | 1.5803219 | 3.243633 |
| Phase-1 RCT-152 | 1.4524632 | 1.2017066 | 1.9963161 | 2.355323 | 1.9570973 | 1.5423272 | 2.1089528 |
| Phase-1 RCT-123 | 0.9052902 | 0.9078858 | 0.9830166 | 0.8830653 | 0.7151989 | 0.6827516 | 0.8648182 |
| Paraoxonase 1 | 0.5347782 | 0.5291951 | 0.4964779 | 0.385524 | 0.8234946 | 0.8659638 | 0.5130099 |
| Organic anion transporter 3 | 0.4603137 | 0.7554135 | 0.5816147 | 0.5460985 | 0.5568534 | 0.8209532 | 0.6551391 |
| N-hydroxy-2-acetylaminofluorene | 0.3833998 | 0.591341 | 0.2920545 | 0.1775276 | 0.2617291 | 0.9447964 | 0.1936473 |
| sulfotransferase (ST1C1) | | | | | | | |
| Integrin beta1 | 1.8082483 | 1.395662 | 1.6570011 | 3.0067053 | 1.4571774 | 0.9084934 | 1.4271086 |
| Insulin-like growth factor binding protein 3 | 0.8875309 | 0.5707315 | 0.5952323 | 0.4059143 | 0.5928716 | 0.9888482 | 0.4820632 |
| ID-1 | 1.7284766 | 1.0038857 | 1.0378671 | 1.3883882 | 1.899624 | 1.2480067 | 1.7147872 |
| Hepatic lipase | 0.5264224 | 0.523139 | 0.4276267 | 0.3047015 | 0.5689814 | 0.6298997 | 0.3937492 |
| Heme oxygenase | 1.8073334 | 1.1162314 | 1.1188248 | 13.707437 | 12.234272 | 1.4714108 | 15.824095 |
| Equilbrative nitrobenzylthioinosine- | 0.4333482 | 0.6654703 | 0.3984554 | 0.2583559 | 0.6318381 | 1.0943241 | 0.3293659 |
| sensitive nucleoside transporter | | | | | | | |
| Epidermal growth factor | 0.8612546 | 0.7556627 | 0.7312049 | 0.693541 | 0.7592656 | 0.7116759 | 0.8039903 |
| c-myc | 2.0439997 | 1.1497276 | 1.4966376 | 3.2799692 | 2.472861 | 1.148268 | 2.9690804 |
| Carbonic anhydrase III | 0.1095224 | 0.3307657 | 0.1265729 | 0.0492532 | 0.1991069 | 0.734624 | 0.0672107 |
| Beta-actin | 1.1259688 | 3.5961053 | 2.6481993 | 9.479847 | 2.485753 | 1.8162844 | 2.6084833 |
| Bax (alpha) | 1.2590859 | 1.2847227 | 1.3057766 | 1.6206344 | 1.53616 | 1.4138767 | 1.3273505 |
| Alpha 1 - inhibitor III | 0.400532 | 0.4712479 | 0.4695987 | 0.2941065 | 0.5751943 | 0.7883048 | 0.3547583 |
| 60S ribosomal protein L6 | 1.3815914 | 1.3096782 | 2.123485 | 2.389527 | 2.092782 | 1.6295863 | 1.941105 |
| Phase-1 RCT-117 | 0.6353955 | 0.851885 | 0.6941977 | 0.3823803 | 0.6683247 | 0.7014307 | 0.7437483 |
| Phase-1 RCT-102 | 0.2420064 | 0.4853712 | 0.3991945 | 0.273727 | 0.4860783 | 0.7413092 | 0.3645808 |
| 3-hydroxyisobutyrate dehydrogenase | 0.5042369 | 0.8092589 | 0.6875293 | 0.5180745 | 0.8750661 | 1.3389322 | 0.692699 |
| ATPase inhibitor (rat mitochondrial IF1 | 0.5540678 | 0.5233417 | 0.4906648 | 0.1857083 | 0.4241819 | 1.1981533 | 0.2625256 |
| protein) | | | | | | | |
| Alpha-2-macroglobulin | 5.5666614 | 0.5487143 | 0.4873345 | 0.3550618 | 0.5548293 | 0.5466803 | 0.4589775 |
| Phase-1 RCT-137 | 0.5771172 | 0.7199864 | 0.8264345 | 0.5868363 | 0.8422374 | 1.1021416 | 0.6023281 |
| Phase-1 RCT 252 | 0.5765711 | 0.7904627 | 0.6729267 | 0.6256322 | 0.7880457 | 1.3286471 | 0.4957551 |
| Phase-1 RCT-65 | 1.3190266 | 1.4980252 | 1.382386 | 1.7829182 | 1.7056478 | 1.2298528 | 1.8561968 |
| Liver fatty acid binding protein | 0.5790002 | 0.4243076 | 0.4268295 | 0.2289972 | 0.211336 | 0.6389216 | 0.2431244 |
| Carbamyl phosphate synthetase I | 0.5582502 | 0.7735812 | 0.7076374 | 0.6953312 | 0.6284652 | 1.2940674 | 0.4797069 |
| c-jun | 2.479193 | 0.8064514 | 0.7868825 | 1.7165539 | 1.81939 | 1.494286 | 2.2417455 |
| Transthyretin | 0.42805 | 0.4270209 | 0.4937554 | 0.3400368 | 0.5179648 | 0.873577 | 0.3216097 |
| Aflatoxin B1 aldehyde reductase | 0.6831096 | 3.6868024 | 4.6069784 | 5.763395 | 17.990175 | 6.454098 | 10.386393 |
| Phase-1 RCT-15 | 1.414294 | 1.3389817 | 1.3332016 | 1.4287503 | 2.3522725 | 1.4210466 | 2.1551485 |
| Insulin-like growth factor I | 0.6504226 | 0.4598514 | 0.4564228 | 0.2201336 | 0.4044988 | 0.979214 | 0.4577577 |
| Sodium/bile acid cotransporter | 0.504769 | 0.7684667 | 0.590177 | 0.8857865 | 0.3977225 | 0.8805587 | 0.5153731 |
| Organic cation transporter 3 | 1.3100312 | 1.2145855 | 1.6628273 | 2.0643818 | 1.2409198 | 0.9546853 | 1.3648101 |
| Gadd153 | 1.0169462 | 1.1502702 | 1.2340784 | 2.2302 | 3.3716147 | 1.5049493 | 2.2389214 |
| Phase-1 RCT-109 | 1.1113037 | 1.4227433 | 2.054258 | 2.2905674 | 1.8951125 | 1.6514163 | 2.225969 |
| Phase-1 RCT-88 | 1.0261106 | 0.5934322 | 0.6891446 | 0.4013787 | 0.7127862 | 0.7500188 | 0.8397243 |
| Ubiquitin conjugating enzyme (RAD 6 | 1.340387 | 0.9548995 | 1.2822496 | 1.5581443 | 1.181682 | 0.8016861 | 1.2199864 |
| homologue) | | | | | | | |
| Glucose-regulated protein 78 | 1.927097 | 1.9487857 | 2.01674 | 1.914075 | 1.4046295 | 1.6247623 | 1.6351714 |
| Ribosomal protein L13A | 1.0597126 | 1.6068325 | 2.3484156 | 2.58199 | 2.0141196 | 1.8109642 | 2.098743 |
| Monocyte chemotactic protein receptor | 1.2631332 | 1.1936095 | 1.2818788 | 1.7275008 | 1.1238714 | 0.9934967 | 1.0638407 |
| (CCR2) | | | | | | | |
| Fas antigen | 1.122157 | 1.770481 | 1.6183406 | 2.1619198 | 0.9198995 | 0.914214 | 1.1204231 |
| Calpactin I heavy chain | 1.0833197 | 1.3471949 | 1.2824762 | 1.6923957 | 1.6149654 | 1.1124085 | 2.3545263 |
| Uncoupling protein 2 | 0.8867982 | 1.2012374 | 1.1107566 | 1.4618201 | 1.1171757 | 0.9873468 | 1.1027135 |
| Phase-1 RCT-168 | 0.9666907 | 0.8054422 | 0.740085 | 0.4710133 | 0.6820972 | 0.7985564 | 0.5664898 |
| Phase-1 RCT-154 | 1.0281553 | 1.1261101 | 1.3269429 | 2.225595 | 0.9985927 | 1.123611 | 1.3041155 |
| Superoxide dismutase Mn | 1.4414591 | 1.2740895 | 1.1780099 | 1.9311266 | 2.6753087 | 1.6109605 | 3.0746365 |
| Phase-1 RCT-214 | 0.950311 | 0.7129448 | 0.7249225 | 0.4325742 | 0.4191459 | 0.7327386 | 0.3835465 |
| Phase-1 RCT-225 | 1.3423305 | 1.2314672 | 1.6224287 | 2.506791 | 3.0578728 | 1.4904069 | 5.0766406 |
| Phase-1 RCT-181 | 0.8490373 | 0.9223343 | 0.911076 | 0.6749479 | 0.8932419 | 0.9997166 | 0.8526726 |
| Nucleoside diphosphate kinase beta | 1.2140932 | 1.1852125 | 1.5279102 | 1.5158077 | 1.8620404 | 1.5043029 | 1.5406747 |
| isoform | | | | | | | |
| Extracellular-signal-regulated kinase 1 | 0.1539685 | 0.4519831 | 0.4073488 | 0.2609656 | 1.4137326 | 1.4518502 | 0.541703 |
| {Ribosomal protein L6} | 1.2686127 | 1.3793004 | 1.9782478 | 2.3206892 | 2.9642246 | 2.14093 | 2.5383334 |
| Aldehyde dehydrogenase, microsomal | 0.9248651 | 0.9191349 | 0.9006553 | 0.6021829 | 0.6092783 | 1.0701761 | 0.4619529 |
| p53 | 1.1080066 | 0.8978914 | 1.1756155 | 1.5839317 | 1.4482548 | 1.1330049 | 1.7004056 |
| Phase-1 RCT-239 | 0.8346975 | 1.2476604 | 1.3921679 | 0.6524429 | 0.8165832 | 0.6827516 | 0.74724 |
| Macrophage inflammatory protein-2 alpha | 2.2808068 | 0.9978087 | 1.0500547 | 2.499662 | 2.9151096 | 1.2480948 | 4.625348 |
| IgE binding protein | 0.9520804 | 1.117225 | 1.130881 | 1.5275583 | 1.4561076 | 1.4163593 | 3.1583354 |
| Phase-1 RCT-205 | 1.3163437 | 1.0250242 | 1.0660911 | 1.7927418 | 1.3004432 | 0.817663 | 2.0407875 |
| Melanoma-associated antigen ME491 | 1.1600649 | 1.010508 | 1.1142567 | 1.6561793 | 1.3884083 | 0.9209077 | 1.6703471 |
| Phase-1 RCT-242 | 2.0941517 | 1.0413784 | 1.3467473 | 1.9358487 | 0.937412 | 0.6155365 | 1.6592388 |
| Enolase alpha | 1.2345902 | 1.3821361 | 2.6187184 | 2.30822 | 2.4191656 | 1.5347193 | 1.7118771 |
| Thymosin beta-10 | 0.9687337 | 1.2853549 | 1.3834659 | 1.7880654 | 1.3312045 | 1.3671318 | 1.3729193 |
| Cofilin | 1.1302248 | 0.9080669 | 0.9679489 | 1.2610892 | 2.9232223 | 2.0638804 | 2.9351072 |
| Carbonyl reductase | 1.2493975 | 1.2182206 | 1.1435828 | 1.9915422 | 1.6783226 | 1.0354006 | 1.3749789 |
| MAP kinase kinase | 1.1272953 | 1.0170603 | 1.243314 | 1.3610886 | 1.2697535 | 1.1657861 | 1.0921427 |
| Phase-1 RCT-162 | 1.0509548 | 1.0332949 | 1.4218295 | 1.4760032 | 1.1428767 | 1.0488986 | 1.2562795 |
| Phase-1 RCT-72 | 1.1354774 | 1.1274544 | 1.0831252 | 1.6762582 | 1.0791714 | 0.8665959 | 1.4422833 |
| Ribosomal protein S8 | 1.081011 | 1.1406254 | 1.8639046 | 2.0553908 | 2.6569045 | 2.2159958 | 2.3907204 |
| Proliferating cell nuclear antigen gene | 1.0237067 | 0.8849356 | 0.9137754 | 1.6138308 | 1.304061 | 0.7641907 | 1.2227778 |
| Phase-1 RCT-192 | 1.0033683 | 1.0690824 | 1.6059343 | 1.8123558 | 1.3094614 | 1.2500607 | 1.596408 |
| IkB-a | 1.1590693 | 1.2678796 | 1.6619941 | 1.7599643 | 2.5236042 | 1.2421546 | 2.260655 |
| Endogenous retroviral sequence, 5′ and | 1.0850494 | 1.113703 | 1.1120529 | 1.6476983 | 1.8541503 | 1.5852102 | 2.5818267 |
| 3′ LTR | | | | | | | |
| Phase-1 RCT-37 | 1.069543 | 1.2586483 | 1.5245445 | 1.7040033 | 1.1191857 | 0.9672426 | 1.2238449 |
| Matrix metalloproteinase-1 | 0.7107794 | 1.4103115 | 1.5858266 | 2.4469833 | 1.5846175 | 1.2634045 | 1.6283784 |
| Cyclin G | 1.6551875 | 1.2089612 | 1.5694448 | 3.6281564 | 2.8484836 | 1.0317866 | 1.7417202 |
| DNA polymerase beta | 0.9724906 | 0.8811477 | 1.2231832 | 1.5396671 | 1.3693786 | 1.2056513 | 1.3069941 |
| Phase-1 RCT-127 | 0.9696277 | 0.957852 | 0.9900196 | 1.4723707 | 1.2133967 | 0.734734 | 1.894941 |
| Multidrug resistant protein-2 | 3.757892 | 0.8502675 | 0.9750508 | 1.8589225 | 1.8614948 | 1.2608433 | 1.6566137 |
| Ribosomal protein S9 | 1.244653 | 1.0825166 | 1.5427363 | 1.6151308 | 1.820619 | 1.2715254 | 1.5436891 |
| Phase-1 RCT-12 | 1.0487278 | 1.3183348 | 1.0848241 | 1.8844128 | 1.1355038 | 1.0028685 | 1.3398657 |
| Beta-tubulin, class I | 1.1703591 | 1.6460553 | 1.4552431 | 4.005797 | 2.6675942 | 1.7822949 | 2.3386688 |
| Argininosuccinate lyase | 1.4711878 | 1.2045664 | 1.905453 | 2.6725478 | 2.651008 | 1.7640364 | 2.1451428 |
| Elongation factor-1 alpha | 0.9303584 | 1.174363 | 1.5331705 | 1.7455474 | 2.0541768 | 1.7279829 | 1.7999846 |
| Phase-1 RCT-55 | 1.7594391 | 1.5802335 | 1.294871 | 4.823493 | 1.5138822 | 0.7434616 | 1.071235 |
| Macrophage inflammatory protein-1 alpha | 1.2852738 | 1.1579158 | 1.1376076 | 1.6846969 | 1.6349049 | 1.1966815 | 4.726845 |
| c-H-ras | 1.3179317 | 1.11818 | 1.0687729 | 0.9092059 | 1.3103396 | 1.3002511 | 1.3093609 |
| Multidrug resistant protein-1 | 3.4660316 | 1.2409593 | 1.2022458 | 1.889119 | 0.6573524 | 1.4860818 | 2.0744915 |
| T-cell cyclophilin | 0.9499963 | 0.8845878 | 1.3215508 | 1.4912754 | 1.6295474 | 1.48192 | 1.5220398 |
| Phase-1 RCT-185 | 0.6019089 | 0.5075305 | 0.4368403 | 0.3225052 | 1.0430269 | 1.2157815 | 0.6016344 |
| 25-DX | 0.818226 | 1.303814 | 1.6666863 | 1.8967818 | 1.968819 | 1.7628139 | 1.8597203 |
| Ref-1 | 1.2543631 | 1.8499584 | 2.1119766 | 3.6170287 | 2.2013197 | 1.237567 | 2.060152 |
| Apolipoprotein CIII | 0.7830294 | 0.7020162 | 0.7529976 | 0.4639558 | 0.8567624 | 0.8125963 | 0.7006609 |
| NADPH quinone oxidoreductase-1 (DT- | 1.6788375 | 2.5108132 | 2.7155702 | 4.842179 | 8.310358 | 3.1774642 | 3.8901808 |
| diaphorase) | | | | | | | |
| Pyruvate kinase, muscle | 1.4259942 | 1.0151799 | 0.9969 | 1.8328559 | 1.8136168 | 1.1696442 | 1.7775879 |
| | BRB | BRB | BRB | CCL4 | CCL4 | CCL4 | CCL4 |
| Compound-Dose (2) | 800 | 800 | 800 | 250 | 250 | 250 | 1000 |
| Animal Number (3) | 2334 | 2335 | 2336 | 2044 | 2045 | 2046 | 2054 |
| Liver Toxicity Necrosis Classification (4) | yes | yes | yes | yes | yes | yes | yes |
| Gene Name (5) | | | | | | | |
| Phase-1 RCT-78 | 0.5752027 | 0.6921904 | 0.6621078 | 0.7273467 | 0.6213893 | 0.7615805 | 0.5578116 |
| Gamma-actin, cytoplasmic | 4.1591353 | 3.7512927 | 3.3873024 | 1.2095281 | 1.7285603 | 1.6833653 | 2.8565161 |
| Matrin F/G | 0.4314714 | 0.4295422 | 0.6078947 | 0.8256525 | 0.6948065 | 0.7947183 | 0.5748441 |
| Zinc finger protein | 3.93518 | 2.1527803 | 2.0759542 | 1.0779959 | 1.3184633 | 1.451205 | 1.6967394 |
| Cathepsin L, sequence 2 | 3.4436734 | 2.803804 | 1.9413538 | 1.9321318 | 2.8458443 | 1.6459104 | 4.7669606 |
| Gadd45 | 3.1520169 | 4.261273 | 2.9653523 | 2.248084 | 2.5185716 | 1.8911576 | 2.030087 |
| Phase-1 RCT-144 | 3.0924978 | 2.98277 | 2.1063964 | 1.6360735 | 2.4618635 | 1.5400269 | 2.627093 |
| Phase-1 RCT-145 | 2.7501855 | 1.9597641 | 1.8462274 | 1.4606414 | 1.8941183 | 1.338319 | 1.7534618 |
| Phase-1 RCT-50 | 2.437746 | 2.5367506 | 1.7603701 | 1.1990254 | 1.3749977 | 1.2915208 | 1.7924391 |
| Phase-1 RCT-92 | 0.4760896 | 0.6497421 | 0.7002124 | 0.6411897 | 0.5980486 | 0.6062883 | 0.3008638 |
| Phase-1 RCT-89 | 0.4543584 | 0.4619704 | 0.4722505 | 0.7737981 | 0.5787484 | 0.6880977 | 0.4605808 |
| 14-3-3 zeta | 1.7873095 | 1.4164262 | 1.4146473 | 1.6601833 | 1.9235764 | 1.7558154 | 2.1649923 |
| Dynamin-1 (D100) | 0.7266259 | 0.7237747 | 0.785397 | 0.7967314 | 0.5810335 | 0.7390643 | 0.4372683 |
| Insulin-like growth factor binding protein 1 | 2.3378346 | 2.3525052 | 1.6197916 | 1.4225075 | 1.6904492 | 1.6052614 | 2.908188 |
| L-gulono-gamma-lactone oxidase | 0.30361 | 0.3259944 | 0.3524514 | 0.545702 | 0.3449596 | 0.5416257 | 0.2873588 |
| Ornithine decarboxylase | 2.9204438 | 2.9021356 | 2.0018406 | 1.2287626 | 1.5142168 | 1.4534425 | 2.0043225 |
| PAR interacting protein | 3.5541978 | 2.9899216 | 2.3397791 | 1.2514662 | 1.6109548 | 1.1780342 | 1.735508 |
| Phase-1 RCT-128 | 0.5684197 | 0.5639549 | 0.6000258 | 0.7659346 | 0.5352064 | 0.6882388 | 0.3094651 |
| Phase-1 RCT-180 | 1.726077 | 1.4324504 | 1.5145471 | 1.1033005 | 1.3904638 | 1.1378132 | 1.2913755 |
| Phase-1 RCT-182 | 0.5041409 | 0.5600268 | 0.6572339 | 0.8337055 | 0.5663141 | 0.6692163 | 0.3597855 |
| Phase-1 RCT-207 | 3.8262067 | 2.7535558 | 2.4641056 | 1.3596162 | 1.7780957 | 1.5105662 | 2.085857 |
| Phase-1 RCT-213 | 2.020875 | 2.3159952 | 1.6054727 | 1.1618257 | 1.6575667 | 1.4549432 | 1.98844 |
| Phase-1 RCT-256 | 0.3752354 | 0.4195517 | 0.4306509 | 0.8142406 | 0.6795092 | 0.8008171 | 0.6382412 |
| Phase-1 RCT-258 | 1.6499089 | 1.3859353 | 1.3581933 | 1.1403744 | 1.4555807 | 1.2219895 | 1.2566491 |
| Phase-1 RCT-264 | 0.3546316 | 0.4998993 | 0.3552938 | 0.5555585 | 0.3693471 | 0.5559151 | 0.2087074 |
| Phase-1 RCT-271 | 0.5889154 | 0.6220582 | 0.6733743 | 1.1132439 | 1.0335475 | 0.9693409 | 0.9136132 |
| Phase-1 RCT-288 | 0.3510526 | 0.3914847 | 0.4038461 | 0.6979315 | 0.5772716 | 0.6385961 | 0.4221105 |
| Phase-1 RCT-38 | 0.4296269 | 0.4023878 | 0.4535689 | 0.7514929 | 0.681133 | 0.8069179 | 0.5871208 |
| Phase-1 RCT-39 | 1.5554736 | 1.3992668 | 1.3427883 | 1.3219073 | 1.5532203 | 1.2589144 | 1.8212497 |
| Phase-1 RCT-68 | 2.0502522 | 1.8923519 | 1.5186543 | 1.2813715 | 1.6150508 | 1.354518 | 1.6562225 |
| Phase-1 RCT-33 | 0.6975865 | 0.5632752 | 0.5756664 | 0.830595 | 0.7000065 | 0.9247553 | 0.4824108 |
| Phase-1 RCT-36 | 0.8444893 | 0.7359124 | 0.7781704 | 0.9182926 | 0.8019388 | 0.9601123 | 0.6381915 |
| 17-beta hydroxysteroid dehydrogenase, | 0.2857581 | 0.3547711 | 0.3188101 | 1.0544912 | 0.5508419 | 0.7560377 | 0.5476907 |
| type 2 | | | | | | | |
| Senescence marker protein-30 | 0.1397427 | 0.1646018 | 0.1474399 | 0.7864495 | 0.5048464 | 0.8299255 | 0.2057194 |
| Ribosomal protein S17 | 2.610419 | 2.6124575 | 1.9685427 | 1.826195 | 2.6640375 | 2.1287396 | 3.4611833 |
| Phase-1 RCT-83 | 0.6019073 | 0.6858119 | 0.5634226 | 0.8265174 | 0.5964726 | 0.5295879 | 0.324705 |
| Phase-1 RCT-49 | 2.4779747 | 2.0622022 | 1.6578346 | 1.5952138 | 1.9354672 | 1.9686404 | 2.4883013 |
| Phase-1 RCT-48 | 0.6157624 | 0.6329519 | 0.5825535 | 1.0240002 | 0.8904838 | 1.1131779 | 0.9803025 |
| Phase-1 RCT-40 | 0.4256537 | 0.4951533 | 0.4450129 | 1.0163147 | 0.8328738 | 0.943199 | 0.7596103 |
| Phase-1 RCT-296 | 0.3838739 | 0.2700433 | 0.2604672 | 0.801514 | 0.3479689 | 0.5475329 | 0.2278402 |
| Phase-1 RCT-295 | 1.7500443 | 2.0645165 | 1.5197922 | 1.511794 | 1.9423338 | 1.5794472 | 2.1096287 |
| Phase-1 RCT-291 | 0.6663093 | 0.7459604 | 0.7320361 | 0.8281298 | 0.7628834 | 0.770144 | 0.556074 |
| Phase-1 RCT-270 | 0.445837 | 0.4777595 | 0.5260442 | 0.8129141 | 0.7105103 | 0.7193425 | 0.6099452 |
| Phase-1 RCT-241 | 2.0646985 | 1.1284157 | 1.1560014 | 1.1818763 | 1.7323387 | 1.208828 | 1.4213411 |
| Phase-1 RCT-191 | 3.0565724 | 2.43825 | 2.0816464 | 1.1923363 | 1.285228 | 1.3083432 | 1.5659757 |
| Phase-1 RCT-189 | 0.3470025 | 0.5071123 | 0.4286053 | 0.9424717 | 0.9339378 | 0.9192929 | 0.6771604 |
| Phase-1 RCT-179 | 3.810576 | 3.6206627 | 2.7567697 | 1.9198552 | 2.8708673 | 2.0569987 | 3.016563 |
| Phase-1 RCT-152 | 2.4927106 | 2.6961086 | 1.9290539 | 1.7668012 | 2.2064083 | 1.7363951 | 2.5763526 |
| Phase-1 RCT-123 | 0.8588566 | 0.8559161 | 0.882353 | 0.9175123 | 0.8728483 | 0.8936751 | 0.8079407 |
| Paraoxonase 1 | 0.5322185 | 0.5038561 | 0.5214765 | 0.6162599 | 0.4003065 | 0.4798027 | 0.2401041 |
| Organic anion transporter 3 | 0.6715305 | 0.5793031 | 0.5761443 | 1.0507448 | 0.6634789 | 0.797854 | 0.6055329 |
| N-hydroxy-2-acetylaminofluorene | 0.2469128 | 0.3123144 | 0.1862634 | 0.6638923 | 0.3774681 | 0.4406919 | 0.1915873 |
| sulfotransferase (ST1C1) | | | | | | | |
| Integrin beta1 | 1.3365039 | 1.3892285 | 1.0067935 | 1.8243084 | 2.457875 | 2.2698157 | 3.9932542 |
| Insulin-like growth factor binding protein 3 | 0.5371005 | 0.5354 | 0.6565966 | 0.728156 | 0.5984591 | 0.8346462 | 0.6476178 |
| ID-1 | 1.4837141 | 1.3515973 | 1.493505 | 1.2739114 | 1.4758488 | 1.4039727 | 1.7147999 |
| Hepatic lipase | 0.4554594 | 0.4118915 | 0.395276 | 0.4727295 | 0.4905481 | 0.4535916 | 0.2903165 |
| Heme oxygenase | 17.7012 | 19.530851 | 12.751645 | 1.3645637 | 1.3536984 | 1.4587083 | 2.906616 |
| Equilbrative nitrobenzylthioinosine- | 0.3405506 | 0.4673865 | 0.3753173 | 0.7540149 | 0.4894777 | 0.497182 | 0.3135049 |
| sensitive nucleoside transporter | | | | | | | |
| Epidermal growth factor | 0.8884125 | 0.8061791 | 0.8542899 | 0.9592204 | 0.8488313 | 0.975965 | 0.8326508 |
| c-myc | 3.041558 | 2.5274923 | 1.8903915 | 1.7368491 | 2.5379696 | 2.16114 | 2.5014267 |
| Carbonic anhydrase III | 0.0350561 | 0.042924 | 0.0538435 | 0.4888808 | 0.1467115 | 0.2420225 | 0.0525565 |
| Beta-actin | 1.7813826 | 1.4456668 | 1.3981987 | 1.3508722 | 1.8152386 | 1.8299372 | 2.4132357 |
| Bax (alpha) | 1.5367341 | 1.711964 | 1.3536209 | 1.1555853 | 1.7218331 | 1.6893425 | 1.8403347 |
| Alpha 1 - inhibitor III | 0.4737816 | 0.4140768 | 0.3244623 | 0.4707834 | 0.3299195 | 0.4359179 | 0.2221123 |
| 60S ribosomal protein L6 | 2.2685843 | 2.1275542 | 1.6857865 | 1.9216183 | 2.717312 | 2.0010726 | 2.9744966 |
| Phase-1 RCT-117 | 0.5236848 | 0.5964308 | 0.6859626 | 0.7427272 | 0.750322 | 0.8017681 | 0.5110082 |
| Phase-1 RCT-102 | 0.3051889 | 0.457208 | 0.4357674 | 0.4328129 | 0.2904431 | 0.3562244 | 0.3224378 |
| 3-hydroxyisobutyrate dehydrogenase | 0.6361281 | 0.6817229 | 0.6778889 | 0.9628395 | 0.896633 | 0.8497854 | 0.7285075 |
| ATPase inhibitor (rat mitochondrial IF1 | 0.2879721 | 0.4119476 | 0.2455653 | 0.6559237 | 0.4648694 | 0.5600308 | 0.263981 |
| protein) | | | | | | | |
| Alpha-2-macroglobulin | 0.4384453 | 0.3634355 | 0.3529321 | 0.5201425 | 0.3928302 | 0.4718215 | 0.2826875 |
| Phase-1 RCT-137 | 0.678141 | 0.6309618 | 0.6101241 | 0.7955334 | 0.8088439 | 0.8133885 | 0.468707 |
| Phase-1 RCT 252 | 0.5783957 | 0.6004942 | 0.5781944 | 0.6841975 | 0.5683702 | 0.8375272 | 0.9835436 |
| Phase-1 RCT-65 | 1.3086895 | 1.6228485 | 1.4601291 | 1.1004561 | 1.3844131 | 1.4342732 | 1.7559551 |
| Liver fatty acid binding protein | 0.2057973 | 0.269358 | 0.2770498 | 0.8223031 | 0.3716559 | 0.6075453 | 0.2655559 |
| Carbamyl phosphate synthetase I | 0.4519629 | 0.5350342 | 0.5663119 | 0.6757115 | 0.5887523 | 0.8789212 | 1.0761185 |
| c-jun | 1.6165622 | 1.7982428 | 1.683729 | 1.5443863 | 1.4943736 | 1.6576997 | 2.5113218 |
| Transthyretin | 0.2723222 | 0.3988705 | 0.3180504 | 0.5036249 | 0.3672069 | 0.5626366 | 0.1890438 |
| Aflatoxin B1 aldehyde reductase | 18.297672 | 12.890534 | 17.715115 | 1.0110922 | 1.10118 | 1.0548302 | 1.0229733 |
| Phase-1 RCT-15 | 1.5146521 | 1.6600058 | 1.4127495 | 1.2006369 | 1.3214173 | 1.3862375 | 1.8812186 |
| Insulin-like growth factor I | 0.4274809 | 0.4245945 | 0.3851804 | 0.6155213 | 0.5350848 | 0.7420573 | 0.531087 |
| Sodium/bile acid cotransporter | 0.4754221 | 0.4255522 | 0.5112941 | 0.812742 | 0.6196334 | 0.5913997 | 0.287901 |
| Organic cation transporter 3 | 1.8386086 | 1.7346933 | 1.5131766 | 1.8688475 | 2.7241354 | 2.0412061 | 2.7727273 |
| Gadd153 | 2.0062053 | 2.3236132 | 1.4319441 | 2.959046 | 4.2869616 | 3.3898818 | 3.5861967 |
| Phase-1 RCT-109 | 2.7550473 | 2.6292863 | 2.1088378 | 1.4029858 | 1.5362656 | 1.6456294 | 2.334362 |
| Phase-1 RCT-88 | 0.6534348 | 0.8179628 | 0.6949046 | 0.7267021 | 0.8002564 | 0.7079529 | 0.3625099 |
| Ubiquitin conjugating enzyme (RAD 6 | 1.4184495 | 1.2268589 | 1.1267122 | 1.7646707 | 2.2154086 | 1.7520143 | 2.1827717 |
| homologue) | | | | | | | |
| Glucose-regulated protein 78 | 2.4844098 | 1.5967646 | 1.2436281 | 2.3805609 | 3.3145213 | 2.1784077 | 2.8014333 |
| Ribosomal protein L13A | 2.626895 | 2.7269235 | 2.050664 | 1.4629867 | 1.6332295 | 1.9092027 | 2.5460644 |
| Monocyte chemotactic protein receptor | 1.1292646 | 1.2517979 | 1.0993181 | 1.9182367 | 2.565654 | 1.9805297 | 2.521138 |
| (CCR2) | | | | | | | |
| Fas antigen | 1.1380218 | 0.9398895 | 1.0850316 | 1.52409 | 1.9021442 | 2.065827 | 2.1579413 |
| Calpactin I heavy chain | 1.3790123 | 1.5433321 | 1.0533175 | 3.2110634 | 4.891623 | 3.199242 | 7.390908 |
| Uncoupling protein 2 | 0.8595521 | 0.8863238 | 0.9150327 | 1.5685462 | 2.0801551 | 2.0898886 | 2.4565792 |
| Phase-1 RCT-168 | 0.6472492 | 0.7333869 | 0.7518598 | 0.7792743 | 0.7212805 | 0.6865891 | 0.4996205 |
| Phase-1 RCT-154 | 1.4506433 | 1.4066342 | 1.1777606 | 1.7960582 | 2.0882967 | 1.4901533 | 2.0881333 |
| Superoxide dismutase Mn | 2.0558743 | 1.9492795 | 1.6398455 | 2.0185843 | 2.8676095 | 2.311271 | 3.2306366 |
| Phase-1 RCT-214 | 0.3348278 | 0.4672513 | 0.466694 | 0.7729123 | 0.7017478 | 0.6794359 | 0.3932969 |
| Phase-1 RCT-225 | 5.506353 | 2.5713568 | 2.0740738 | 2.5432196 | 4.2953467 | 2.862378 | 5.27724 |
| Phase-1 RCT-181 | 0.9840422 | 0.8573403 | 0.8822259 | 0.7945542 | 0.6792496 | 0.8048474 | 0.5870315 |
| Nucleoside diphosphate kinase beta | 1.5954394 | 1.4057313 | 1.1744483 | 1.902188 | 2.390527 | 1.9011159 | 3.0686514 |
| isoform | | | | | | | |
| Extracellular-signal-regulated kinase 1 | 0.498213 | 0.6802634 | 0.8466522 | 0.5877713 | 0.2783755 | 0.3886987 | 0.2470829 |
| {Ribosomal protein L6} | 2.3996828 | 2.6032498 | 1.912428 | 1.6424365 | 2.2052822 | 1.9591738 | 2.881203 |
| Aldehyde dehydrogenase, microsomal | 0.7377977 | 0.721162 | 0.8002882 | 0.9071515 | 0.7147374 | 0.8689016 | 0.5509729 |
| p53 | 1.4421312 | 1.3626181 | 1.1512202 | 1.2743765 | 1.6323931 | 1.2273897 | 1.2768244 |
| Phase-1 RCT-239 | 0.8103561 | 0.9172621 | 0.9213235 | 0.7128952 | 0.4546853 | 0.6657475 | 0.4769034 |
| Macrophage inflammatory protein-2 alpha | 1.2691606 | 1.2484365 | 1.1192974 | 1.6413306 | 2.3680766 | 2.2864835 | 3.227502 |
| IgE binding protein | 1.3620456 | 1.3220685 | 1.097769 | 2.3807302 | 2.453882 | 2.3325307 | 3.1056318 |
| Phase-1 RCT-205 | 1.553151 | 1.5094599 | 1.3233093 | 1.2626331 | 1.7128332 | 1.2395712 | 1.6625992 |
| Melanoma-associated antigen ME491 | 1.3275331 | 1.1962161 | 0.9172469 | 1.8410482 | 2.1732683 | 1.6214827 | 3.367168 |
| Phase-1 RCT-242 | 1.250377 | 1.1579976 | 1.1252191 | 1.3389702 | 1.7030543 | 1.3996145 | 2.2257814 |
| Enolase alpha | 1.609621 | 1.7265801 | 1.2798468 | 1.5611404 | 2.6199589 | 2.0889735 | 2.5944748 |
| Thymosin beta-10 | 1.1893088 | 1.5480845 | 1.2779149 | 1.4730594 | 1.8665379 | 1.8632579 | 2.247151 |
| Cofilin | 2.6319299 | 2.0498528 | 1.7689312 | 1.4253789 | 1.7304277 | 1.3533752 | 1.52941 |
| Carbonyl reductase | 1.6399505 | 1.1494583 | 1.2750872 | 1.3252776 | 1.5711792 | 1.4042662 | 1.6659124 |
| MAP kinase kinase | 1.1160479 | 1.319969 | 1.2237319 | 1.3970004 | 1.4562615 | 1.5401709 | 1.5831045 |
| Phase-1 RCT-162 | 1.5536621 | 1.6090195 | 1.2900134 | 1.5771856 | 2.0801594 | 1.6942228 | 2.3559554 |
| Phase-1 RCT-72 | 1.4668568 | 1.4730872 | 1.2249998 | 1.4337312 | 1.8212488 | 1.5552975 | 2.5087173 |
| Ribosomal protein S8 | 2.4551883 | 2.4836323 | 1.7998819 | 1.8157563 | 2.639998 | 2.056113 | 2.9762309 |
| Proliferating cell nuclear antigen gene | 1.2340511 | 1.1312876 | 1.1130136 | 1.5914425 | 1.6220983 | 1.7251031 | 1.855107 |
| Phase-1 RCT-192 | 1.830081 | 1.8559958 | 1.453505 | 1.706817 | 2.3180552 | 2.220798 | 2.7666936 |
| IkB-a | 2.0875418 | 2.405251 | 1.739983 | 1.4321122 | 2.1734576 | 1.8087915 | 2.7720058 |
| Endogenous retroviral sequence, 5′ and | 2.6395524 | 1.482342 | 1.0769471 | 1.5203727 | 2.199482 | 1.8691419 | 2.8587914 |
| 3′ LTR | | | | | | | |
| Phase-1 RCT-37 | 1.5688986 | 1.7192701 | 1.2856841 | 1.5247991 | 1.8626586 | 1.7855766 | 2.236209 |
| Matrix metalloproteinase-1 | 1.8037354 | 1.4908485 | 1.3651168 | 1.3976161 | 1.9154376 | 1.3202015 | 2.7234108 |
| Cyclin G | 1.8202311 | 1.857407 | 1.29541 | 2.0753772 | 3.015346 | 2.5912278 | 4.071453 |
| DNA polymerase beta | 1.5408971 | 1.4638617 | 1.0994608 | 1.3717923 | 1.6941034 | 1.3432182 | 1.7196708 |
| Phase-1 RCT-127 | 1.7495538 | 1.5254116 | 1.2732198 | 2.1441424 | 2.1044838 | 2.0956986 | 2.044238 |
| Multidrug resistant protein-2 | 1.391584 | 1.5416601 | 1.4478899 | 2.018425 | 2.1274397 | 2.136595 | 3.4459193 |
| Ribosomal protein S9 | 1.8866477 | 1.4525287 | 1.347097 | 1.2346561 | 1.9368812 | 1.6728492 | 2.0256555 |
| Phase-1 RCT-12 | 1.767338 | 1.583604 | 1.5427805 | 1.5911512 | 2.0895646 | 1.9405565 | 2.8219137 |
| Beta-tubulin, class I | 2.2925663 | 2.1714432 | 1.9644219 | 2.0693703 | 3.1523101 | 2.5183637 | 4.3570747 |
| Argininosuccinate lyase | 2.2517676 | 2.6117756 | 1.8981912 | 1.3301924 | 1.3644207 | 1.65502 | 1.4428127 |
| Elongation factor-1 alpha | 2.2146778 | 2.0406666 | 1.5377442 | 1.855479 | 2.3757195 | 1.8430456 | 2.9945145 |
| Phase-1 RCT-55 | 1.3164384 | 1.3526459 | 1.3015441 | 1.0967561 | 1.4820198 | 1.5222796 | 2.465852 |
| Macrophage inflammatory protein-1 alpha | 1.204815 | 1.0863261 | 1.2111582 | 1.3438146 | 2.5230424 | 1.8127491 | 2.3230808 |
| c-H-ras | 1.2833016 | 1.242251 | 1.0674042 | 1.2223715 | 1.3737329 | 1.2664496 | 1.633201 |
| Multidrug resistant protein-1 | 1.8535577 | 2.0215464 | 1.461586 | 1.7051295 | 2.147129 | 2.101333 | 2.671373 |
| T-cell cyclophilin | 1.7381636 | 1.6747843 | 1.1900076 | 1.4157008 | 1.6543496 | 1.2602824 | 1.6785198 |
| Phase-1 RCT-185 | 0.4785779 | 0.5230842 | 0.4984501 | 0.8182678 | 0.6041049 | 0.5918355 | 0.3293772 |
| 25-DX | 3.5727997 | 2.8209481 | 2.6559925 | 0.9414572 | 0.903716 | 0.8580595 | 0.9903556 |
| Ref-1 | 1.996669 | 2.1912048 | 1.7677178 | 1.2987827 | 1.5457338 | 1.1408149 | 1.272425 |
| Apolipoprotein CIII | 0.743617 | 0.6846948 | 0.7071428 | 0.6265475 | 0.5124873 | 0.6200687 | 0.383803 |
| NADPH quinone oxidoreductase-1 (DT- | 4.1818104 | 5.28765 | 3.9087167 | 0.9557092 | 0.9555533 | 0.6538534 | 0.4922176 |
| diaphorase) | | | | | | | |
| Pyruvate kinase, muscle | 0.894228 | 1.0612112 | 0.9770695 | 1.6428086 | 2.2181032 | 2.1696622 | 3.636856 |
| | CCL4 | CCL4 | DMN | DMN | DMN | LPS | LPS |
| Compound-Dose (2) | 1000 | 1000 | 20 | 20 | 20 | 8 | 8 |
| Animal Number (3) | 2055 | 2056 | 1754 | 1755 | 1756 | 354 | 355 |
| Liver Toxicity Necrosis Classification (4) | yes | yes | yes | yes | yes | yes | yes |
| Gene Name (5) | | | | | | | |
| Phase-1 RCT-78 | 0.5866493 | 0.5619331 | 0.6976997 | 0.7401357 | 0.6312013 | 0.5656012 | 0.7713856 |
| Gamma-actin, cytoplasmic | 3.0457294 | 3.9200022 | 2.1755028 | 2.3958645 | 1.820712 | 6.400891 | 2.6559298 |
| Matrin F/G | 0.4180156 | 0.422786 | 0.6148238 | 0.6690309 | 0.6657567 | 0.3655312 | 0.6086198 |
| Zinc finger protein | 2.9112062 | 2.2035537 | 2.226279 | 2.6996589 | 2.332279 | 4.824339 | 1.4791515 |
| Cathepsin L, sequence 2 | 5.0101705 | 7.0921206 | 2.2908368 | 2.0969257 | 2.4150183 | 4.4302115 | 2.4644918 |
| Gadd45 | 4.1851983 | 1.5416964 | 2.0483875 | 2.1846497 | 2.416181 | 2.7726345 | 1.1335245 |
| Phase-1 RCT-144 | 2.8818934 | 2.2322137 | 1.4190725 | 1.5668545 | 1.4747944 | 1.6095161 | 1.4690024 |
| Phase-1 RCT-145 | 1.9948301 | 1.7656795 | 1.34305 | 1.4387385 | 1.341065 | 2.1710186 | 1.5790329 |
| Phase-1 RCT-50 | 2.380688 | 1.7364157 | 1.8440611 | 2.034157 | 2.052821 | 2.4425428 | 1.1325245 |
| Phase-1 RCT-92 | 0.25695 | 0.2166238 | 0.4548766 | 0.5576299 | 0.4595501 | 0.1824703 | 0.4798026 |
| Phase-1 RCT-89 | 0.3352208 | 0.4085257 | 0.5909707 | 0.6109154 | 0.5832527 | 0.3703634 | 0.7264777 |
| 14-3-3 zeta | 2.4876626 | 2.5851805 | 2.5322504 | 2.5469873 | 2.3486543 | 1.8583727 | 1.726598 |
| Dynamin-1 (D100) | 0.3681365 | 0.3156565 | 0.6928623 | 0.6317583 | 0.5881891 | 0.4311712 | 0.5552758 |
| Insulin-like growth factor binding protein 1 | 4.09912 | 2.6876137 | 1.5428771 | 1.2521566 | 1.75171 | 9.570809 | 3.1451118 |
| L-gulono-gamma-lactone oxidase | 0.2836435 | 0.3368473 | 0.109222 | 0.1410183 | 0.1024532 | 0.1540088 | 0.4284025 |
| Ornithine decarboxylase | 2.6702077 | 1.7596312 | 3.36143 | 3.1633654 | 3.3053238 | 2.5982585 | 1.7417487 |
| PAR interacting protein | 1.8047488 | 1.5374928 | 1.3036879 | 1.372397 | 1.3069781 | 1.5500888 | 1.3725723 |
| Phase-1 RCT-128 | 0.2631531 | 0.1966537 | 0.5546628 | 0.4105176 | 0.4124066 | 0.1287209 | 0.2610953 |
| Phase-1 RCT-180 | 1.3830785 | 1.4211651 | 1.3999335 | 1.3392003 | 1.3569623 | 1.3504307 | 1.0581591 |
| Phase-1 RCT-182 | 0.3219927 | 0.3130553 | 0.4731438 | 0.5696933 | 0.4702373 | 0.1998336 | 0.4732614 |
| Phase-1 RCT-207 | 2.323234 | 2.052179 | 4.962027 | 5.8372107 | 4.2311134 | 2.3718715 | 1.6958535 |
| Phase-1 RCT-213 | 1.8683666 | 1.7128414 | 1.4119722 | 1.3509684 | 1.3226017 | 1.9665909 | 1.912038 |
| Phase-1 RCT-256 | 0.4909451 | 0.6471754 | 0.4204458 | 0.4681548 | 0.3985326 | 0.1783197 | 0.4952782 |
| Phase-1 RCT-258 | 1.2575247 | 1.1695766 | 1.2801701 | 1.2076132 | 1.1705775 | 1.4595515 | 1.5391624 |
| Phase-1 RCT-264 | 0.2154877 | 0.188911 | 0.5761324 | 0.5465801 | 0.479608 | 0.2527308 | 0.4882877 |
| Phase-1 RCT-271 | 0.8366982 | 0.8164485 | 0.4656967 | 0.4626961 | 0.4087006 | 0.2605189 | 0.3692878 |
| Phase-1 RCT-288 | 0.3186414 | 0.2719188 | 0.5253037 | 0.5078079 | 0.4492259 | 0.3661428 | 0.7278074 |
| Phase-1 RCT-38 | 0.4150598 | 0.533973 | 0.4111636 | 0.4577699 | 0.4010142 | 0.1925091 | 0.4667193 |
| Phase-1 RCT-39 | 2.2124736 | 1.7496692 | 1.8224922 | 1.6815362 | 1.8016862 | 1.654823 | 1.328566 |
| Phase-1 RCT-68 | 2.1566505 | 1.4050556 | 1.485298 | 1.418502 | 1.3690349 | 1.7326883 | 1.5665108 |
| Phase-1 RCT-33 | 0.3803582 | 0.4745723 | 0.6986424 | 0.5836725 | 0.56718 | 0.372041 | 0.5170196 |
| Phase-1 RCT-36 | 0.5533384 | 0.6301913 | 0.7939576 | 0.7302828 | 0.7118964 | 0.6817357 | 0.7909334 |
| 17-beta hydroxysteroid dehydrogenase, | 1.0615338 | 1.0238155 | 0.368348 | 0.368322 | 0.3759525 | 0.4504577 | 0.3096664 |
| type 2 | | | | | | | |
| Senescence marker protein-30 | 0.2534248 | 0.1735427 | 0.2483918 | 0.2251867 | 0.2134901 | 0.0657977 | 0.8578165 |
| Ribosomal protein S17 | 1.8915443 | 3.1056142 | 1.2711122 | 1.3190625 | 1.2238346 | 3.3450155 | 2.8750076 |
| Phase-1 RCT-83 | 0.4341747 | 0.5129233 | 0.9146224 | 0.6623862 | 0.7000412 | 0.4333007 | 0.74153 |
| Phase-1 RCT-49 | 2.9415908 | 1.8672976 | 1.2631255 | 1.3293079 | 1.5471046 | 1.5203938 | 1.1185461 |
| Phase-1 RCT-48 | 0.9350863 | 1.022386 | 0.5052744 | 0.5270482 | 0.4756997 | 0.8411301 | 1.1232914 |
| Phase-1 RCT-40 | 0.8049822 | 0.6798528 | 0.7428639 | 0.7489824 | 0.6309288 | 0.4652426 | 0.8891092 |
| Phase-1 RCT-296 | 0.1987942 | 0.3182876 | 0.2677555 | 0.2315752 | 0.2159824 | 0.2116805 | 0.3070078 |
| Phase-1 RCT-295 | 2.6815648 | 1.945779 | 1.7784998 | 1.7093782 | 1.5794578 | 1.7463839 | 1.7317485 |
| Phase-1 RCT-291 | 0.4585504 | 0.4680468 | 0.6175751 | 0.6251997 | 0.6205142 | 0.3430022 | 0.6811923 |
| Phase-1 RCT-270 | 0.5490685 | 0.6973921 | 0.3661616 | 0.4698817 | 0.3580332 | 0.7376154 | 0.9222267 |
| Phase-1 RCT-241 | 1.8536006 | 1.1864719 | 1.8973739 | 1.6643655 | 1.9321384 | 4.4410043 | 2.4815652 |
| Phase-1 RCT-191 | 1.2354523 | 1.5771224 | 1.4348435 | 1.6863945 | 1.4465715 | 1.4370487 | 1.0701076 |
| Phase-1 RCT-189 | 0.5598978 | 0.4924721 | 0.7783914 | 0.7727737 | 0.7230405 | 0.9204566 | 0.9100975 |
| Phase-1 RCT-179 | 4.3181944 | 3.5845747 | 1.8037996 | 1.7831013 | 1.6343557 | 2.1989865 | 2.131917 |
| Phase-1 RCT-152 | 3.8669693 | 2.2573147 | 1.7310591 | 1.6679004 | 1.6254013 | 1.952817 | 1.7218956 |
| Phase-1 RCT-123 | 0.8191747 | 0.7673098 | 0.8486443 | 0.8801969 | 0.8450431 | 0.8232431 | 0.9170819 |
| Paraoxonase 1 | 0.3058035 | 0.2891786 | 0.3929069 | 0.3948471 | 0.3838238 | 0.2013049 | 0.3522886 |
| Organic anion transporter 3 | 0.6113555 | 0.6187644 | 0.6130382 | 0.6605445 | 0.8027735 | 0.4978685 | 0.4288849 |
| N-hydroxy-2-acetylaminofluorene | 0.2415767 | 0.4231103 | 0.484724 | 0.4197559 | 0.3222497 | 0.1877245 | 0.8197165 |
| sulfotransferase (ST1C1) | | | | | | | |
| Integrin beta1 | 5.1019516 | 3.8175101 | 1.573644 | 1.4625477 | 1.8222693 | 2.8472126 | 1.6182631 |
| Insulin-like growth factor binding protein 3 | 0.6303501 | 0.6314594 | 0.4924185 | 0.5146573 | 0.4497303 | 0.8378819 | 0.8384919 |
| ID-1 | 1.6156043 | 1.7759207 | 1.6622672 | 1.7349473 | 1.674152 | 1.9203006 | 1.7582985 |
| Hepatic lipase | 0.2398743 | 0.3034584 | 0.4596221 | 0.3347099 | 0.3740014 | 0.2268487 | 0.283025 |
| Heme oxygenase | 3.1921563 | 2.1816916 | 1.9657408 | 1.2491082 | 1.7900773 | 7.5731807 | 4.1159506 |
| Equilbrative nitrobenzylthioinosine- | 0.3744075 | 0.4208692 | 0.6519004 | 0.5300047 | 0.4478541 | 0.3818915 | 0.702141 |
| sensitive nucleoside transporter | | | | | | | |
| Epidermal growth factor | 0.7741652 | 0.7305021 | 0.8157427 | 0.8084115 | 0.720866 | 0.9693316 | 1.7297313 |
| c-myc | 2.5362456 | 1.9832413 | 1.4373343 | 1.4835546 | 1.8225677 | 1.8932587 | 1.1154914 |
| Carbonic anhydrase III | 0.0736234 | 0.0395167 | 0.1195262 | 0.2709458 | 0.0702452 | 0.0117874 | 0.2131445 |
| Beta-actin | 1.9375596 | 3.9194362 | 3.757428 | 3.3873239 | 3.3316884 | 4.541738 | 3.5038154 |
| Bax (alpha) | 2.1037614 | 2.1600115 | 2.8567157 | 2.5559995 | 3.241329 | 1.9627922 | 1.3635134 |
| Alpha 1 - inhibitor III | 0.2710247 | 0.2268792 | 0.309813 | 0.2609946 | 0.1764347 | 0.2364945 | 0.217445 |
| 60S ribosomal protein L6 | 3.3545399 | 3.5781336 | 1.7731404 | 1.7175888 | 1.5630764 | 2.5147152 | 2.0600655 |
| Phase-1 RCT-117 | 0.3997174 | 0.3334382 | 0.7746869 | 0.8133801 | 0.7481362 | 0.9192369 | 1.1283714 |
| Phase-1 RCT-102 | 0.2470088 | 0.4453755 | 0.2258873 | 0.2477862 | 0.1698049 | 0.5152615 | 0.4322599 |
| 3-hydroxyisobutyrate dehydrogenase | 0.6972511 | 0.6349042 | 0.7004641 | 0.6557396 | 0.651206 | 0.4262131 | 0.8733965 |
| ATPase inhibitor (rat mitochondrial IF1 | 0.2955565 | 0.1477015 | 0.4832205 | 0.45668 | 0.3451347 | 0.3854721 | 1.1122072 |
| protein) | | | | | | | |
| Alpha-2-macroglobulin | 0.3910856 | 0.2752153 | 0.4048126 | 0.3395929 | 0.244181 | 23.249428 | 41.42365 |
| Phase-1 RCT-137 | 0.434687 | 0.3112053 | 0.5307291 | 0.4360122 | 0.4262447 | 0.6077146 | 1.0373673 |
| Phase-1 RCT 252 | 0.6941654 | 0.8083181 | 0.5263416 | 0.584892 | 0.4145114 | 0.3861008 | 0.8064206 |
| Phase-1 RCT-65 | 1.872127 | 1.6192725 | 1.8160166 | 1.5001667 | 2.1914096 | 1.343297 | 1.1698955 |
| Liver fatty acid binding protein | 0.3382239 | 0.2494568 | 0.3516527 | 0.3246081 | 0.3006346 | 0.1161629 | 0.6079174 |
| Carbamyl phosphate synthetase I | 0.7903598 | 0.8374881 | 0.512184 | 0.5761676 | 0.4066467 | 0.3663031 | 0.7933183 |
| c-jun | 2.5174065 | 2.6855447 | 2.5841537 | 2.7955966 | 4.314467 | 2.6335511 | 0.7459129 |
| Transthyretin | 0.2011055 | 0.1376162 | 0.4308786 | 0.4424237 | 0.36734 | 0.1902513 | 0.3039445 |
| Aflatoxin B1 aldehyde reductase | 0.7426355 | 0.844063 | 0.9466383 | 0.6902703 | 0.4967341 | 0.3211476 | 0.554858 |
| Phase-1 RCT-15 | 1.6929237 | 1.5214922 | 1.6199026 | 1.558452 | 1.7254446 | 1.0915115 | 1.0193505 |
| Insulin-like growth factor I | 0.499285 | 0.5132347 | 0.5977248 | 0.5840488 | 0.514194 | 1.1351922 | 1.0237365 |
| Sodium/bile acid cotransporter | 0.2600418 | 0.1927289 | 0.2757223 | 0.3455292 | 0.3803255 | 0.0984281 | 0.1745389 |
| Organic cation transporter 3 | 3.160059 | 3.1794884 | 1.6465837 | 1.6556724 | 1.4823074 | 2.0686288 | 1.8236203 |
| Gadd153 | 3.9715643 | 3.3575413 | 3.5105417 | 3.7394226 | 3.718889 | 2.250709 | 2.37944 |
| Phase-1 RCT-109 | 2.4580665 | 2.476058 | 1.5446198 | 1.5333225 | 1.4501681 | 2.0901756 | 1.6830128 |
| Phase-1 RCT-88 | 0.4308733 | 0.2647123 | 0.6943669 | 0.7229518 | 0.7016216 | 0.3865782 | 0.7791432 |
| Ubiquitin conjugating enzyme (RAD 6 | 2.5353518 | 2.467456 | 1.4764893 | 1.400529 | 1.3854331 | 2.283681 | 2.0152547 |
| homologue) | | | | | | | |
| Glucose-regulated protein 78 | 3.1048079 | 3.077932 | 1.876616 | 1.4548755 | 1.8926141 | 2.6247435 | 3.4789073 |
| Ribosomal protein L13A | 2.8906465 | 3.3036292 | 1.8384963 | 1.690393 | 1.6658877 | 3.598756 | 2.4913232 |
| Monocyte chemotactic protein receptor | 2.926744 | 2.8152988 | 1.2009089 | 1.3419428 | 1.518025 | 1.6373835 | 1.5220052 |
| (CCR2) | | | | | | | |
| Fas antigen | 2.2001362 | 3.0729573 | 1.6169696 | 1.5319164 | 1.4695637 | 2.590346 | 2.3735297 |
| Calpactin I heavy chain | 12.612631 | 5.756459 | 1.7486068 | 1.533049 | 1.928503 | 2.9384553 | 2.3833342 |
| Uncoupling protein 2 | 2.747504 | 3.2889676 | 1.4044496 | 1.4367484 | 1.6971748 | 6.955903 | 4.3691616 |
| Phase-1 RCT-168 | 0.4832274 | 0.4702842 | 0.5985751 | 0.5447391 | 0.49088 | 0.4847646 | 0.7845792 |
| Phase-1 RCT-154 | 2.6354098 | 2.5956717 | 3.5815625 | 3.7613585 | 3.2897666 | 2.6428416 | 2.0896 |
| Superoxide dismutase Mn | 4.810452 | 3.3345268 | 3.1344278 | 3.0455792 | 3.6470962 | 28.04024 | 7.832339 |
| Phase-1 RCT-214 | 0.3511649 | 0.3579352 | 0.5273634 | 0.4848814 | 0.4199829 | 0.3492123 | 0.6440124 |
| Phase-1 RCT-225 | 4.8842797 | 7.39835 | 1.8376107 | 1.8109711 | 2.8840644 | 2.2128696 | 1.0646118 |
| Phase-1 RCT-181 | 0.5210405 | 0.5422825 | 0.6564993 | 0.7138847 | 0.6847149 | 0.6005467 | 0.7634349 |
| Nucleoside diphosphate kinase beta | 3.1656315 | 2.5818887 | 1.6010534 | 1.6418049 | 1.6392882 | 1.6041514 | 1.6604731 |
| isoform | | | | | | | |
| Extracellular-signal-regulated kinase 1 | 0.2955306 | 0.2157069 | 0.6341048 | 0.6378844 | 0.5643207 | 0.1133004 | 0.2310077 |
| {Ribosomal protein L6} | 3.3378031 | 2.7102234 | 1.5689124 | 1.56034 | 1.4911461 | 2.3021507 | 1.8350469 |
| Aldehyde dehydrogenase, microsomal | 0.3863192 | 0.4810944 | 0.7647163 | 0.7006017 | 0.6312822 | 0.3849141 | 0.7131299 |
| p53 | 1.5792546 | 1.3919713 | 1.5384244 | 1.6138364 | 1.4117432 | 1.5930196 | 1.5543827 |
| Phase-1 RCT-239 | 0.4623943 | 0.6159899 | 0.6604329 | 0.7436845 | 0.7570936 | 0.5096839 | 0.7600747 |
| Macrophage inflammatory protein-2 alpha | 4.3032336 | 4.131834 | 5.5702376 | 3.9283495 | 5.477772 | 5.18203 | 1.0923767 |
| IgE binding protein | 4.228803 | 2.5187447 | 1.4950185 | 1.5629817 | 2.0334957 | 3.794436 | 2.5900264 |
| Phase-1 RCT-205 | 2.1216888 | 1.7425519 | 1.3302377 | 1.1805426 | 1.1610519 | 10.008086 | 1.9198421 |
| Melanoma-associated antigen ME491 | 3.3384204 | 3.0576618 | 1.5299679 | 1.5107981 | 1.3708565 | 3.208101 | 2.3447323 |
| Phase-1 RCT-242 | 2.502959 | 1.9304981 | 2.65577 | 2.743458 | 3.0169485 | 2.733232 | 1.0198131 |
| Enolase alpha | 3.401915 | 2.7248144 | 1.442403 | 1.6410878 | 1.3387332 | 1.8455384 | 1.9848325 |
| Thymosin beta-10 | 2.712783 | 3.1536043 | 1.6251298 | 1.7323841 | 1.5989083 | 3.9025621 | 2.8668551 |
| Cofilin | 1.6249001 | 1.6070769 | 1.5602291 | 1.6471423 | 1.6590159 | 1.8075478 | 1.4177225 |
| Carbonyl reductase | 1.922179 | 1.7614437 | 1.3840626 | 1.5300332 | 1.3416725 | 1.7387627 | 1.2162352 |
| MAP kinase kinase | 1.9318287 | 1.3715352 | 1.0449365 | 0.9797045 | 1.0009618 | 1.600833 | 1.4160094 |
| Phase-1 RCT-162 | 2.5117955 | 2.3354561 | 1.1536458 | 1.1076671 | 1.0317303 | 1.5802153 | 1.7163582 |
| Phase-1 RCT-72 | 2.8982408 | 2.14235 | 1.499445 | 1.5753512 | 1.3181504 | 1.5916523 | 1.1150602 |
| Ribosomal protein S8 | 3.6680622 | 2.810254 | 1.4871483 | 1.504773 | 1.441238 | 2.3676322 | 2.2928112 |
| Proliferating cell nuclear antigen gene | 1.6779164 | 1.7010555 | 1.235963 | 1.3882531 | 1.3174995 | 1.3985983 | 1.1826684 |
| Phase-1 RCT-192 | 3.1902063 | 2.960657 | 1.4751972 | 1.4764692 | 1.5566491 | 1.0021596 | 1.355259 |
| IkB-a | 3.2208064 | 3.0759237 | 1.2719116 | 1.1730907 | 1.1193483 | 1.798759 | 1.3409535 |
| Endogenous retroviral sequence, 5′ and | 2.5319226 | 4.017355 | 1.1651069 | 1.4002427 | 1.8155566 | 2.8396678 | 1.2753332 |
| 3′ LTR | | | | | | | |
| Phase-1 RCT-37 | 2.3224986 | 2.2411325 | 1.2078261 | 1.2474034 | 1.2350786 | 1.3788408 | 1.4027919 |
| Matrix metalloproteinase-1 | 2.4765117 | 2.7124596 | 1.6149306 | 1.4061809 | 1.9094181 | 2.8414335 | 2.7873578 |
| Cyclin G | 5.3807287 | 3.834693 | 14.440476 | 13.8836155 | 11.29682 | 4.8585463 | 2.08717 |
| DNA polymerase beta | 1.9210663 | 1.7829913 | 1.2404811 | 1.2879065 | 1.1820363 | 1.656545 | 1.5768837 |
| Phase-1 RCT-127 | 2.7382896 | 1.4555861 | 1.1976109 | 1.315999 | 1.270272 | 1.5563499 | 1.181267 |
| Multidrug resistant protein-2 | 5.6216693 | 4.368329 | 6.537999 | 5.2299156 | 6.291526 | 6.101459 | 1.3631802 |
| Ribosomal protein S9 | 2.353816 | 2.4559047 | 1.3804723 | 1.2748027 | 1.2205178 | 2.6673539 | 2.3430028 |
| Phase-1 RCT-12 | 2.8943422 | 2.7650568 | 1.3748502 | 1.5280583 | 1.4395126 | 1.0252221 | 1.1662626 |
| Beta-tubulin, class I | 4.5409718 | 4.0335603 | 1.5651003 | 1.6743562 | 1.3539561 | 1.160093 | 1.4484888 |
| Argininosuccinate lyase | 1.5525362 | 1.7829258 | 1.3006063 | 1.2219703 | 1.2713649 | 2.1828194 | 1.2965101 |
| Elongation factor-1 alpha | 3.4633143 | 3.0697136 | 1.2317096 | 1.1826649 | 1.0801109 | 1.4001266 | 1.5824631 |
| Phase-1 RCT-55 | 2.497205 | 3.481534 | 1.0522933 | 1.0909011 | 0.9684229 | 2.6684349 | 1.4018466 |
| Macrophage inflammatory protein-1 alpha | 3.662614 | 2.820711 | 1.0940967 | 1.1576011 | 1.2802014 | 2.5488615 | 2.3011167 |
| c-H-ras | 1.5157272 | 1.2626487 | 2.1059296 | 1.908223 | 1.8544047 | 1.533287 | 1.7200844 |
| Multidrug resistant protein-1 | 4.672183 | 3.5507157 | 6.194462 | 4.565507 | 6.861969 | 6.61696 | 1.8740501 |
| T-cell cyclophilin | 1.8808334 | 1.6533729 | 1.1197162 | 1.1597918 | 0.9988607 | 1.7894582 | 1.7653552 |
| Phase-1 RCT-185 | 0.3651064 | 0.3859086 | 0.6561667 | 0.7295263 | 0.5573542 | 0.3613886 | 0.3755098 |
| 25-DX | 0.7308176 | 0.7980366 | 0.8732745 | 0.7882463 | 0.7198932 | 0.5672101 | 0.6674811 |
| Ref-1 | 1.7280709 | 1.0760912 | 1.3972272 | 1.3010856 | 1.40766 | 1.0615592 | 0.5916336 |
| Apolipoprotein CIII | 0.3645007 | 0.3544948 | 0.6036383 | 0.6460893 | 0.5554969 | 0.4126337 | 0.5027271 |
| NADPH quinone oxidoreductase-1 (DT- | 0.6578077 | 0.3876311 | 1.5965279 | 0.9436654 | 1.3620995 | 0.6428888 | 0.3372252 |
| diaphorase) | | | | | | | |
| Pyruvate kinase, muscle | 2.452139 | 4.8594093 | 1.1102097 | 0.9760528 | 1.164582 | 5.7231703 | 3.6357646 |

| | LPS | TET | TET | TET |
|---|---|---|---|---|
| Compound-Dose (2) | 8 | 150 | 150 | 150 |
| Animal Number (3) | 356 | 1244 | 1245 | 1246 |
| Liver Toxicity Necrosis Classification (4) | yes | yes | yes | yes |
| Gene Name (5) | | | | |
| Phase-1 RCT-78 | 0.9501752 | 0.696658 | 0.704728 | 0.6924388 |
| Gamma-actin, cytoplasmic | 1.7433479 | 2.0713866 | 1.8656448 | 2.314668 |
| Matrin F/G | 0.7306065 | 0.6552458 | 0.5341573 | 0.5309179 |
| Zinc finger protein | 1.481397 | 1.0913143 | 1.8628906 | 1.0597495 |
| Cathepsin L, sequence 2 | 2.0182147 | 5.017635 | 3.5476038 | 3.706211 |
| Gadd45 | 1.0902935 | 2.2435951 | 2.4440973 | 1.897707 |
| Phase-1 RCT-144 | 1.0102489 | 2.2818992 | 2.7335503 | 1.9791094 |
| Phase-1 RCT-145 | 1.0256796 | 1.9883407 | 2.796879 | 1.557653 |
| Phase-1 RCT-50 | 1.2060434 | 1.1913787 | 1.1956064 | 1.1602687 |
| Phase-1 RCT-92 | 0.5462084 | 0.2600658 | 0.4397287 | 0.47349 |
| Phase-1 RCT-89 | 0.6337453 | 0.7567936 | 0.7044188 | 0.8962625 |
| 14-3-3 zeta | 1.2884848 | 2.0383265 | 1.2585353 | 1.6559125 |
| Dynamin-1 (D100) | 1.7845032 | 0.5021089 | 0.6688958 | 0.7320942 |
| Insulin-like growth factor binding protein 1 | 4.4176188 | 5.5840554 | 14.986245 | 4.352973 |
| L-gulono-gamma-lactone oxidase | 0.7763927 | 0.3512681 | 0.3215035 | 0.4937252 |
| Ornithine decarboxylase | 0.9407112 | 2.3900425 | 3.79146 | 2.4031808 |
| PAR interacting protein | 0.9911812 | 2.3314483 | 3.399191 | 2.498675 |
| Phase-1 RCT-128 | 2.5368717 | 0.4495242 | 0.5020827 | 0.6744867 |
| Phase-1 RCT-180 | 1.2047818 | 1.6687604 | 2.1196127 | 1.5529243 |
| Phase-1 RCT-182 | 0.9002813 | 0.4417826 | 0.5780773 | 0.6721711 |
| Phase-1 RCT-207 | 1.5759715 | 1.4611253 | 1.3495806 | 1.3248067 |
| Phase-1 RCT-213 | 1.2079958 | 1.6935531 | 2.1802568 | 1.4635438 |
| Phase-1 RCT-256 | 0.5465581 | 0.6016132 | 0.4106733 | 0.567182 |
| Phase-1 RCT-258 | 1.030154 | 1.8378503 | 1.6964399 | 1.7165172 |
| Phase-1 RCT-264 | 0.9711127 | 0.573242 | 0.4133744 | 0.5339918 |
| Phase-1 RCT-271 | 0.981989 | 0.5658845 | 0.2291156 | 0.7852426 |
| Phase-1 RCT-288 | 0.8314838 | 0.7927976 | 0.5058951 | 0.7464088 |
| Phase-1 RCT-38 | 0.5035883 | 0.4866596 | 0.392105 | 0.5153089 |
| Phase-1 RCT-39 | 0.9892198 | 1.4951581 | 1.7632052 | 1.5089498 |
| Phase-1 RCT-68 | 1.2695843 | 1.3621974 | 2.1911771 | 1.349382 |
| Phase-1 RCT-33 | 0.5720211 | 0.5256491 | 0.3678081 | 0.692952 |
| Phase-1 RCT-36 | 0.7314478 | 0.6294956 | 0.6062564 | 0.7238615 |
| 17-beta hydroxysteroid dehydrogenase, | 0.7507751 | 0.2374674 | 0.0670459 | 0.3042366 |
| type 2 | | | | |
| Senescence marker protein-30 | 0.4901452 | 0.3265905 | 0.6101742 | 0.4756615 |
| Ribosomal protein S17 | 1.8085343 | 5.932119 | 4.060182 | 4.122136 |
| Phase-1 RCT-83 | 0.4578597 | 0.4806289 | 1.0974122 | 0.4933587 |
| Phase-1 RCT-49 | 1.028222 | 2.5147662 | 3.1215978 | 1.4098959 |
| Phase-1 RCT-48 | 0.6541692 | 1.5474547 | 0.7114306 | 1.2948297 |
| Phase-1 RCT-40 | 0.5999652 | 0.8103631 | 0.7407968 | 0.9397186 |
| Phase-1 RCT-296 | 0.2609357 | 0.3584994 | 0.2410906 | 0.4301213 |
| Phase-1 RCT-295 | 1.4193774 | 2.1300788 | 1.1552148 | 1.9949582 |
| Phase-1 RCT-291 | 0.7058753 | 0.8062863 | 0.902847 | 0.8723913 |
| Phase-1 RCT-270 | 1.4590384 | 0.841916 | 0.5652776 | 1.0668179 |
| Phase-1 RCT-241 | 1.755722 | 1.2062521 | 5.1566067 | 1.2833571 |
| Phase-1 RCT-191 | 0.9604746 | 1.0463908 | 1.1161467 | 1.0215257 |
| Phase-1 RCT-189 | 2.2782378 | 0.775351 | 0.5634856 | 0.9121578 |
| Phase-1 RCT-179 | 1.2312531 | 3.515963 | 3.3964953 | 2.8372438 |
| Phase-1 RCT-152 | 1.2152582 | 3.4195673 | 3.464468 | 3.2163763 |
| Phase-1 RCT-123 | 1.0439477 | 1.0546218 | 0.9203017 | 0.9318368 |
| Paraoxonase 1 | 0.4543921 | 0.6170477 | 0.6435435 | 0.5868695 |
| Organic anion transporter 3 | 0.7824898 | 0.7201845 | 0.2666372 | 0.5907037 |
| N-hydroxy-2-acetylaminofluorene | 0.3250847 | 0.2943599 | 0.2678011 | 0.3148257 |
| sulfotransferase (ST1C1) | | | | |
| Integrin beta1 | 1.9419613 | 3.420329 | 1.4432498 | 3.0726092 |
| Insulin-like growth factor binding protein 3 | 1.1824737 | 1.5775281 | 1.0321959 | 1.2421197 |
| ID-1 | 2.068847 | 1.194348 | 1.195102 | 1.224189 |
| Hepatic lipase | 0.2469568 | 0.3094597 | 0.3313223 | 0.3375785 |
| Heme oxygenase | 3.8494248 | 1.3794575 | 1.7603893 | 0.9527385 |
| Equilbrative nitrobenzylthioinosine- | 0.4337137 | 0.3926923 | 0.378743 | 0.4083329 |
| sensitive nucleoside transporter | | | | |
| Epidermal growth factor | 0.9592072 | 1.5870221 | 0.9880326 | 1.2959584 |
| c-myc | 0.8650907 | 2.3030853 | 3.6104088 | 1.6796997 |
| Carbonic anhydrase III | 0.0484569 | 0.042079 | 0.0155059 | 0.0623944 |
| Beta-actin | 1.9381654 | 1.8195095 | 0.9495761 | 1.6375195 |
| Bax (alpha) | 1.0760796 | 1.3634481 | 1.2035679 | 1.2662263 |
| Alpha 1 - inhibitor III | 0.1569447 | 0.4046228 | 0.4373252 | 0.3698592 |
| 60S ribosomal protein L6 | 1.5466884 | 3.6433098 | 2.9608994 | 3.0682132 |
| Phase-1 RCT-117 | 1.0358288 | 1.3469433 | 0.8523306 | 1.003985 |
| Phase-1 RCT-102 | 0.4386782 | 0.5187646 | 0.2228282 | 0.6092138 |
| 3-hydroxyisobutyrate dehydrogenase | 0.9578417 | 1.0383763 | 0.863119 | 1.0147189 |
| ATPase inhibitor (rat mitochondrial IF1 | 0.7471101 | 0.8263711 | 0.6378441 | 0.7316048 |
| protein) | | | | |
| Alpha-2-macroglobulin | 62.307213 | 16.063503 | 1.3831676 | 5.767094 |
| Phase-1 RCT-137 | 0.8031477 | 1.0044543 | 0.8107134 | 0.9401739 |
| Phase-1 RCT 252 | 1.2346245 | 0.3881909 | 0.2774641 | 0.724722 |
| Phase-1 RCT-65 | 1.0036343 | 1.3006688 | 1.4564623 | 1.4998978 |
| Liver fatty acid binding protein | 0.3609023 | 0.367345 | 0.1693196 | 0.1875227 |
| Carbamyl phosphate synthetase I | 1.2678778 | 0.3178494 | 0.2527193 | 0.6347455 |
| c-jun | 0.6617427 | 1.1737827 | 0.8586048 | 0.8595898 |
| Transthyretin | 0.7334244 | 0.4855523 | 0.3420498 | 0.440925 |
| Aflatoxin B1 aldehyde reductase | 0.6119386 | 1.0316564 | 0.4717417 | 0.96732 |
| Phase-1 RCT-15 | 1.3335447 | 0.9537966 | 1.3662827 | 0.9507271 |
| Insulin-like growth factor I | 1.3732512 | 1.519353 | 0.8663654 | 1.1916889 |
| Sodium/bile acid cotransporter | 0.2761393 | 0.2228238 | 0.3779753 | 0.2763225 |
| Organic cation transporter 3 | 1.4894385 | 3.607207 | 2.8135083 | 3.2252228 |
| Gadd153 | 1.2583137 | 4.319167 | 6.1349654 | 3.1438622 |
| Phase-1 RCT-109 | 1.4481655 | 2.7390962 | 1.4874567 | 1.7395225 |
| Phase-1 RCT-88 | 0.6923598 | 0.2878019 | 0.6176607 | 0.3978799 |
| Ubiquitin conjugating enzyme (RAD 6 | 1.7165396 | 3.8817916 | 2.1415613 | 3.087568 |
| homologue) | | | | |
| Glucose-regulated protein 78 | 0.8616164 | 3.1809487 | 1.4576194 | 2.3401244 |
| Ribosomal protein L13A | 1.8317091 | 2.706431 | 1.6055487 | 2.2288036 |
| Monocyte chemotactic protein receptor | 1.3633609 | 3.163511 | 1.6815453 | 2.9708285 |
| (CCR2) | | | | |
| Fas antigen | 1.3982044 | 1.9717069 | 1.2646663 | 1.5546567 |
| Calpactin I heavy chain | 1.8821069 | 1.534087 | 2.0382743 | 1.3811786 |
| Uncoupling protein 2 | 2.8572087 | 2.0109236 | 1.036088 | 1.149882 |
| Phase-1 RCT-168 | 0.7486263 | 0.6611595 | 0.545911 | 0.8431302 |
| Phase-1 RCT-154 | 1.5649568 | 2.04064 | 2.2405696 | 1.6209718 |
| Superoxide dismutase Mn | 2.394923 | 2.8805702 | 1.2925397 | 2.1549284 |
| Phase-1 RCT-214 | 0.5590386 | 0.5834078 | 0.394233 | 0.6990368 |
| Phase-1 RCT-225 | 0.6646972 | 1.0973544 | 0.9136313 | 0.8297837 |
| Phase-1 RCT-181 | 0.9006349 | 0.830143 | 0.8672756 | 0.7856751 |
| Nucleoside diphosphate kinase beta | 1.4666766 | 2.4673586 | 2.24651 | 2.4346757 |
| isoform | | | | |
| Extracellular-signal-regulated kinase 1 | 0.0696844 | 0.1219657 | 0.4694366 | 0.0861044 |
| {Ribosomal protein L6} | 1.5237614 | 2.5557644 | 2.4429827 | 2.9320183 |
| Aldehyde dehydrogenase, microsomal | 0.6943056 | 0.6208947 | 0.4124292 | 0.9013456 |
| p53 | 1.0921013 | 2.1979454 | 2.3337293 | 2.5007539 |
| Phase-1 RCT-239 | 0.6855991 | 0.6996276 | 0.8080244 | 0.7033839 |
| Macrophage inflammatory protein-2 alpha | 0.876895 | 2.1031275 | 1.8884826 | 1.3847436 |
| IgE binding protein | 1.6660497 | 1.551265 | 2.0679865 | 1.1396853 |
| Phase-1 RCT-205 | 1.161093 | 1.3876686 | 1.4977337 | 1.3650494 |
| Melanoma-associated antigen ME491 | 1.5201687 | 1.6761239 | 2.6907697 | 1.1217319 |
| Phase-1 RCT-242 | 1.7555054 | 1.0455018 | 1.8127679 | 1.0365269 |
| Enolase alpha | 2.1774774 | 2.1596978 | 1.8890874 | 2.79597 |
| Thymosin beta-10 | 1.6888744 | 1.2492478 | 1.054894 | 0.9320112 |
| Cofilin | 1.5704997 | 0.9476048 | 2.0307536 | 0.9091924 |
| Carbonyl reductase | 1.1286834 | 1.1547201 | 1.1931348 | 0.8869779 |
| MAP kinase kinase | 1.1958083 | 2.4855893 | 1.4102533 | 1.5998566 |
| Phase-1 RCT-162 | 1.4109371 | 2.5982504 | 1.9126536 | 2.2607555 |
| Phase-1 RCT-72 | 1.5056164 | 0.9813537 | 1.0477194 | 0.9731574 |
| Ribosomal protein S8 | 1.5463511 | 3.8184233 | 3.2701423 | 3.4564333 |
| Proliferating cell nuclear antigen gene | 0.9877722 | 1.5501332 | 1.1627108 | 1.295589 |
| Phase-1 RCT-192 | 1.1878891 | 2.843858 | 2.6253877 | 2.207398 |
| IkB-a | 1.3540832 | 1.452268 | 1.3961512 | 2.0755541 |
| Endogenous retroviral sequence, 5′ and | 2.98493 | 1.0842069 | 0.6936452 | 1.3325577 |
| 3′ LTR | | | | |
| Phase-1 RCT-37 | 1.3393837 | 2.0552845 | 1.607621 | 2.120729 |
| Matrix metalloproteinase-1 | 2.0484884 | 2.2166493 | 1.1281914 | 1.4505272 |
| Cyclin G | 1.4569163 | 2.5818644 | 2.4444077 | 2.3667803 |
| DNA polymerase beta | 1.1382797 | 2.340702 | 1.574351 | 2.2816472 |
| Phase-1 RCT-127 | 1.2497655 | 2.114602 | 2.9832692 | 1.1246674 |
| Multidrug resistant protein-2 | 1.1497924 | 2.29267 | 2.0402007 | 2.0502715 |
| Ribosomal protein S9 | 1.6080464 | 3.9799404 | 3.1277268 | 3.3447332 |
| Phase-1 RCT-12 | 1.0778675 | 1.478154 | 0.9862046 | 1.5113869 |
| Beta-tubulin, class I | 1.0494959 | 1.6342669 | 1.0425073 | 1.6465117 |
| Argininosuccinate lyase | 3.543073 | 1.5434442 | 1.7829134 | 1.8219311 |
| Elongation factor-1 alpha | 1.1562912 | 3.0029964 | 2.1054409 | 3.040404 |
| Phase-1 RCT-55 | 1.106244 | 1.5388008 | 0.9695869 | 1.5653485 |
| Macrophage inflammatory protein-1 alpha | 1.1437613 | 3.3961067 | 1.7024724 | 0.6086369 |
| c-H-ras | 1.4194655 | 2.0273306 | 0.5580133 | 2.4432003 |
| Multidrug resistant protein-1 | 1.2183996 | 2.9116135 | 2.6057546 | 2.4536557 |
| T-cell cyclophilin | 1.1794919 | 2.4148564 | 1.7656102 | 2.4298098 |
| Phase-1 RCT-185 | 0.5059522 | 0.3668154 | 0.4734672 | 0.4554441 |
| 25-DX | 0.7736301 | 0.7046103 | 0.5021314 | 0.7932399 |
| Ref-1 | 0.6284427 | 0.7658656 | 2.0282104 | 1.0856603 |
| Apolipoprotein CIII | 0.7918134 | 0.4094147 | 0.4548712 | 0.4560681 |
| NADPH quinone oxidoreductase-1 (DT- | 0.6055676 | 0.4935458 | 1.157221 | 0.738646 |
| diaphorase) | | | | |
| Pyruvate kinase, muscle | 2.5874655 | 3.2084875 | 0.9033669 | 2.4648018 |

(1) Gene expression data for 24 hour timpoint are presented as mean ratio of treatment/control for all 24 hour predictive genes (Table 5).
(2) Compound and dose abbreviations as in Table 1.
(3) Individual animal number
(4) Liver necrosis classification for compound-dose group at 72 h: yes, necrosis observed; no, no necrosis observed
(5) Predictive gene (as in Table 5 and as included in Table 32)

TABLE 36
Expression Data for 72 Hour Timepoint (1)

| | 5-FU | 5-FU | 5-FU | 5-FU | 5-FU | 5-FU | AFLB |
|---|---|---|---|---|---|---|---|
| Compound-Dose | 13 | 13 | 13 | 50 | 50 | 50 | 1 |
| Animal Number | 1927 | 1928 | 1929 | 1937 | 1938 | 1939 | 154 |
| Liver Toxicity Necrosis Classification | no | no | no | no | no | no | yes |
| Gene Name | | | | | | | |
| Osteoactivin | 0.969951 | 1.0700635 | 1.0865526 | 1.129316 | 1.2050588 | 1.1870325 | 19.701735 |
| Stathmin | 1.0413896 | 1.0483744 | 1.1142105 | 1.1054851 | 1.1851743 | 1.1240172 | 2.5768566 |
| Calpactin I heavy chain | 0.9645633 | 0.88285 | 1.0546522 | 0.9876137 | 0.79915 | 0.8879408 | 6.9543247 |
| Phase-1 RCT-179 | 0.9493563 | 0.9855681 | 0.9642185 | 0.8180571 | 0.9315319 | 0.9270162 | 2.6958725 |
| Phase-1 RCT-107 | 1.0761541 | 1.1578256 | 1.239701 | 1.1342269 | 0.9466655 | 1.0133674 | 0.6435815 |
| Heme oxygenase | 0.9025708 | 1.135419 | 0.8615583 | 0.9341835 | 0.9455808 | 1.1712598 | 0.510144 |
| Collagen type II | 0.7163266 | 0.6514535 | 1.103215 | 1.2716824 | 1.162186 | 1.102798 | 3.0882776 |
| Phase-1 RCT-81 | 0.9795277 | 1.0268575 | 0.8821607 | 0.9235126 | 1.0425531 | 0.9879032 | 1.1211524 |
| Glycine methyltrans-ferase | 1.0341022 | 1.4220868 | 1.3221622 | 0.8464599 | 0.8657392 | 0.6646456 | 0.0819506 |
| Cystatin C | 0.8633391 | 0.7946892 | 0.7379763 | 0.7160083 | 0.6839765 | 0.7305825 | 2.4716213 |
| Melanoma-associated antigen ME491 | 1.145443 | 0.9589542 | 1.06653 | 0.9292073 | 1.1735859 | 0.9954493 | 4.9345407 |
| Phase-1 RCT-146 | 0.9954965 | 0.9688958 | 1.1668619 | 1.1559684 | 1.1536561 | 1.1624548 | 3.5023203 |
| Gamma-actin, cytoplasmic | 0.7517159 | 1.0923398 | 0.7756412 | 1.1703815 | 1.0074474 | 0.8592308 | 5.2881746 |
| Adenine nucleotide translocator 1 | 0.8236181 | 0.6897306 | 0.7170191 | 0.5581313 | 0.623617 | 0.594527 | 1.6180526 |
| Thymosin beta-10 | 0.997547 | 0.8344549 | 0.853404 | 0.7035356 | 0.7076368 | 0.691427 | 3.9618843 |
| Insulin-like growth factor I, exon 6 | 0.6797717 | 0.7061867 | 0.9319299 | 0.7817259 | 0.9670643 | 0.9336631 | 0.2434111 |
| Phase-1 RCT-78 | 1.003851 | 1.1061211 | 1.0524888 | 1.0166066 | 1.2369981 | 0.9863712 | 0.6201779 |
| Alpha-2-microglobulin | 0.7529354 | 0.8843004 | 0.8709653 | 0.6830331 | 0.8158258 | 0.6713768 | 0.1020938 |
| High affinity IgE receptor gamma chain | 0.8997756 | 0.62943 | 0.8488627 | 0.8539848 | 0.7522562 | 0.7487342 | 5.2777414 |
| (FcERIgamma) | | | | | | | |
| 60S ribosomal protein L6 | 0.8167281 | 0.6483287 | 0.648639 | 0.7580135 | 0.7720456 | 0.8397325 | 2.7640116 |
| IgE binding protein | 0.8768226 | 0.7910359 | 0.9715458 | 0.9835239 | 0.7600466 | 0.8918518 | 1.9591208 |
| Phase-1 RCT-149 | 0.9832444 | 1.215046 | 1.0454046 | 1.0081153 | 1.0813608 | 0.8787513 | 1.0148742 |
| Cytochrome P450 2C11 | 0.9164714 | 0.8312135 | 1.0904676 | 0.8027299 | 0.6246012 | 0.9679835 | 0.0701988 |
| Uncoupling protein 2 | 1.0084312 | 0.8977585 | 0.9049379 | 0.9235997 | 0.750951 | 0.7013487 | 1.6111355 |
| Cofilin | 0.8681752 | 0.9315227 | 0.8598644 | 0.8072376 | 0.9125329 | 0.7562591 | 3.6496747 |
| Alpha-tubulin | 1.5154852 | 0.8585563 | 0.8663417 | 0.9582878 | 1.0310849 | 0.9280919 | 4.747834 |
| Stearyl-CoA desaturase, liver | 0.7958847 | 1.5796893 | 0.7592334 | 0.3704651 | 0.3228053 | 0.4370344 | 0.0385444 |
| Glutathione S-transferase theta-1 | 1.0998224 | 1.1397295 | 0.9932352 | 0.7582377 | 0.8401789 | 0.8411005 | 0.6331312 |
| alpha-1,2-fucosyltrans-ferase | 0.7747647 | 0.8443899 | 0.8826416 | 0.9551688 | 0.8185684 | 0.9322889 | 0.607298 |
| Phase-1 RCT-138 | 0.8736028 | 0.984682 | 0.8201236 | 0.8566141 | 0.8652619 | 0.7459089 | 5.375366 |
| Phase-1 RCT-295 | 0.9197255 | 1.1378814 | 1.0468268 | 1.0005407 | 0.9669781 | 1.073701 | 2.440102 |
| Phase-1 RCT-164 | 1.075194 | 1.0326631 | 1.0399846 | 1.0580999 | 1.0442871 | 1.0720721 | 0.3899932 |
| Alpha-2-macroglobulin, sequence 2 | 0.9283375 | 0.944221 | 0.8468411 | 0.9722416 | 0.8175801 | 1.011621 | 2.3466167 |
| Biliverdin reductase | 1.0735621 | 1.1150256 | 1.1032149 | 0.9632576 | 0.9041788 | 1.0130522 | 2.3091648 |
| Phase-1 RCT-121 | 1.0867867 | 0.9979767 | 1.1079133 | 1.0369955 | 0.9455674 | 0.9311594 | 4.4027247 |
| Dynamin-1 (D100) | 1.0033746 | 0.9972388 | 1.0340531 | 1.0128713 | 1.0680677 | 0.8801333 | 0.8436685 |
| Phase-1 RCT-192 | 0.8399137 | 0.9046559 | 0.838625 | 0.8465866 | 0.8506914 | 0.8648363 | 2.1317122 |
| Fatty acid synthase | 1.1757907 | 1.3086183 | 0.647183 | 0.7598658 | 0.6236664 | 0.7148122 | 0.2666054 |
| Phase-1 RCT-211 | 1.0717757 | 1.2180904 | 1.2642843 | 1.1857843 | 1.0922923 | 1.0381172 | 2.2164423 |
| Ribosomal protein S9 | 0.9845772 | 0.8995425 | 0.8423289 | 0.7311903 | 0.8462595 | 0.8115394 | 0.9280109 |
| NADP-dependent isocitrate dehydrogenase, | 0.87452 | 0.8838992 | 0.8225936 | 0.8860645 | 0.9534469 | 0.9433341 | 0.3819105 |
| cytosolic | | | | | | | |
| Phase-1 RCT-57 | 1.2352293 | 1.0792147 | 1.054714 | 1.0451549 | 1.0912895 | 0.921436 | 1.1813148 |
| Phase-1 RCT-36 | 1.0064197 | 1.00379 | 0.9392124 | 1.0398009 | 1.1371857 | 1.1346154 | 0.9181783 |
| Apolipoprotein All | 0.8000239 | 1.0856588 | 0.8252667 | 1.095082 | 0.6929398 | 0.7764528 | 0.0742243 |
| Phase-1 RCT-68 | 1.0339419 | 1.0902889 | 1.1296043 | 1.1040165 | 1.1730129 | 1.1150606 | 2.6457624 |
| Multidrug resistant protein-2 | 1.2035265 | 0.872673 | 1.0378677 | 1.1044017 | 1.0718092 | 1.2825606 | 3.0549223 |
| Phase-1 RCT-39 | 1.1388977 | 1.0998565 | 1.141138 | 1.0590173 | 1.1543058 | 1.5932043 | 2.5275664 |
| NIPK | 0.8863677 | 1.1073897 | 1.1775956 | 1.1853739 | 1.3786619 | 1.2025402 | 1.1588168 |
| F1-ATPase beta subunit | 1.070447 | 0.897666 | 0.7789232 | 0.8946795 | 0.8918327 | 0.8932382 | 0.9210895 |
| Elongation factor-1 alpha | 1.0019583 | 0.850616 | 0.7781481 | 0.8828738 | 0.8602917 | 0.9676453 | 1.4780015 |
| Phase-1 RCT-24 | 1.3823482 | 1.0563383 | 1.0210133 | 1.2867222 | 1.1807474 | 1.1239629 | 4.516335 |
| Beta-tubulin, class I | 1.2002798 | 1.1058043 | 0.9102078 | 1.231496 | 1.1170341 | 0.9742035 | 3.4324992 |
| 14-3-3 zeta | 1.0739676 | 1.0081358 | 0.9577284 | 1.0638145 | 1.0654646 | 1.0558933 | 3.1627436 |
| Alpha-prothymosin | 0.8115841 | 0.8865615 | 0.8561143 | 0.7226828 | 0.9715313 | 0.6856253 | 2.793975 |
| Phase-1 RCT-58 | 1.021573 | 0.928902 | 0.5977446 | 1.2995312 | 0.9395064 | 1.2866535 | 0.3408188 |
| Phase-1 RCT-290 | 0.6342936 | 1.1279619 | 0.8451277 | 0.6937851 | 0.5548385 | 0.8444006 | 0.8274252 |
| Transitional endoplasmic reticulum ATPase | 1.1092666 | 0.9528287 | 0.8217455 | 0.8537489 | 0.882807 | 0.8266731 | 1.2542001 |
| Beta-actin | 0.6016344 | 0.680592 | 0.4757692 | 0.6301505 | 0.5687643 | 0.4876196 | 4.842202 |
| Thioredoxin-1 (Trx1) | 0.7578854 | 0.7573996 | 0.7604263 | 0.6942516 | 0.7556952 | 0.7427477 | 1.5995107 |
| Phase-1 RCT-174 | 1.0368207 | 0.9793743 | 1.005953 | 1.0208279 | 0.9380149 | 0.8928571 | 1.2303116 |
| Beta-actin, sequence 2 | 0.9120035 | 1.075999 | 0.8447301 | 0.9673183 | 0.8791355 | 0.8839514 | 4.3372073 |
| Phase-1 RCT-109 | 0.9689114 | 0.8357195 | 0.8819622 | 0.9127668 | 0.9109769 | 0.8494114 | 2.2023225 |
| Phase-1 RCT-154 | 0.8882586 | 1.0813825 | 0.8955578 | 0.991882 | 0.9421839 | 1.0034672 | 5.1616063 |
| Hypoxanthine-guanine | 0.9622907 | 0.9921408 | 0.7495764 | 0.7036116 | 0.6856603 | 0.6259565 | 1.785463 |
| phosphoribosyltransferase | | | | | | | |
| Phase-1 RCT-213 | 1.0289448 | 1.012547 | 0.9163631 | 0.997773 | 1.0004324 | 0.9399092 | 1.4611111 |
| Phase-1 RCT-260 | 1.0592992 | 1.17589 | 1.1138974 | 1.1777583 | 1.0766671 | 1.9512194 | 0.6897307 |
| Phase-1 RCT-12 | 1.2208481 | 1.1157202 | 1.1148968 | 1.208064 | 1.1607348 | 1.0571896 | 1.467976 |
| Phase-1 RCT-293 | 0.902205 | 0.725246 | 0.8025484 | 0.7797258 | 0.6877668 | 0.6692982 | 3.8202794 |
| Ribosomal protein S8 | 0.7313199 | 0.7608143 | 0.7209135 | 0.7627183 | 0.7604317 | 0.7806864 | 2.8829014 |
| Pyruvate kinase, muscle | 0.9262224 | 0.782378 | 0.921054 | 0.929309 | 0.6203263 | 0.7854532 | 3.3741438 |
| Nucleoside diphosphate kinase beta isoform | 0.9627273 | 1.0238625 | 0.9052532 | 0.9212371 | 0.8906448 | 0.8457137 | 1.534274 |
| p55CDC | 1.0358981 | 1.0604078 | 1.3452958 | 1.1426294 | 1.3883522 | 1.4402821 | 0.8230972 |
| Phase-1 RCT-156 | 1.0753835 | 1.0034877 | 0.9444678 | 0.8924415 | 1.1059723 | 0.92344 | 1.523145 |
| Ribosomal protein L13A | 1 | 0.84786 | 0.8928293 | 0.883473 | 0.8352346 | 0.9050952 | 1.5971133 |
| Phase-1 RCT-258 | 0.9970371 | 1.0217106 | 0.8866961 | 0.980904 | 1.023642 | 0.9953602 | 2.024786 |
| Insulin-like growth factor I | 0.6293313 | 0.7038529 | 0.7230224 | 0.6800474 | 0.7772068 | 0.7673804 | 0.2660137 |
| Cytochrome P450 3A1 | 1.2766204 | 0.6279791 | 1.0958333 | 1.1710782 | 1.057408 | 1.0173821 | 0.3436746 |
| {Ribosomal protein 16} | 0.7747525 | 0.8080512 | 0.7262718 | 0.8239423 | 0.8378456 | 0.8450586 | 2.861291 |
| Organic cation transporter 3 | 0.8651299 | 0.7339278 | 0.8093612 | 0.9530645 | 0.9719352 | 0.9321305 | 2.3108587 |
| Calpain 2 | 0.9167102 | 0.8971285 | 1.054384 | 0.9315786 | 0.9122772 | 0.9271386 | 3.2720208 |
| Phase-1 RCT-102 | 0.5818176 | 0.855484 | 1.0618109 | 0.562142 | 0.5952672 | 0.5575636 | 0.1750601 |
| Equilbrative nitrobenzylthioinosine-sensitive | 0.9058754 | 0.9178214 | 0.8896893 | 0.9612475 | 0.9035993 | 0.9073674 | 0.4296702 |
| nucleoside transporter | | | | | | | |
| Phase-1 RCT-227 | 0.8798 | 1.0533893 | 1.1532979 | 0.8942071 | 1.3262522 | 0.8222188 | 0.5139121 |
| N-hydroxy-2-acetylaminofluorene sulfotransferase (ST1C1) | 0.8193285 | 0.7249482 | 0.8270077 | 0.8645389 | 0.7941526 | 0.7990018 | 0.1475141 |
| Cathepsin S | 0.8819882 | 0.7363666 | 0.7552248 | 0.9229482 | 0.7112028 | 0.7955199 | 3.3119197 |
| Apolipoprotein CIII | 0.9517704 | 1.011242 | 0.8711543 | 0.8812347 | 0.8227258 | 0.7497364 | 0.606379 |
| Cytochrome P450 2A3 | 0.9409711 | 1.2727741 | 1.1126173 | 1.5505565 | 0.9431745 | 1.2375308 | 0.83507 |
| Cytochrome P450 2C23 | 0.6554183 | 0.855616 | 0.9141911 | 0.8563054 | 0.7852808 | 0.8750418 | 0.2044926 |
| Apolipoprotein C1 | 0.6748665 | 1.0760888 | 0.8368316 | 0.752275 | 0.8591685 | 0.7302318 | 0.1033435 |
| Betaine homocysteine methyltransferase (BHMT) | 0.419078 | 1.173935 | 0.8100621 | 0.600515 | 0.470184 | 0.6716519 | 0.0469193 |
| Paraoxonase 1 | 0.7164763 | 0.6322424 | 0.4969536 | 0.7062079 | 0.7205794 | 0.7315334 | 0.167038 |
| Phase-1 RCT-207 | 1.1119056 | 0.8838391 | 0.9812176 | 0.9445025 | 0.960273 | 0.9953586 | 4.8798175 |
| Cathepsin B | 0.9338804 | 0.9600784 | 0.8313867 | 0.8064224 | 0.9005088 | 0.856561 | 3.6256413 |
| Phase-1 RCT-144 | 1.02001 | 0.8740591 | 0.9930726 | 1.03842 | 0.9967744 | 1.0168918 | 2.0809698 |
| Alpha-1 microglobulin/bikunin precursor (Ambp) | 0.8722734 | 0.9848654 | 0.79009 | 0.7826492 | 0.993853 | 0.8752059 | 0.2573969 |
| Cyclin D1 | 0.8593711 | 0.5427309 | 0.8305812 | 0.9992564 | 0.8993177 | 0.8506033 | 3.0825303 |
| Presenilin-1 | 0.6878033 | 0.6949133 | 0.7032819 | 0.7136693 | 0.8378685 | 0.4963172 | 0.1590457 |
| Protein tyrosine phosphatase, receptor type, D | 0.7612171 | 1.2185898 | 0.8442223 | 1.000478 | 1.1400886 | 0.9831977 | 0.2163785 |
| Multidrug resistant protein-1 | 1.16106 | 0.8565732 | 1.2746483 | 1.3562694 | 1.2849736 | 1.5758476 | 4.341694 |
| Cathepsin L, sequence 2 | 1.1103283 | 0.8557289 | 0.9555453 | 1.1334109 | 1.0747747 | 1.5177153 | 3.2009592 |
| JNK1 stress activated protein kinase | 0.9671518 | 1.0813558 | 1.0258964 | 0.9366986 | 1.1199747 | 1.9125434 | 0.6564403 |
| Phase-1 RCT-43 | 1.1149504 | 0.9930238 | 0.9940934 | 0.9903299 | 1.0845349 | 0.9372512 | 1.5595812 |
| Tissue plasminogen activator | 0.9461408 | 0.9378151 | 1.0214618 | 1.203015 | 1.0096819 | 1.0228758 | 2.1652613 |
| Ubiquitin conjugating enzyme (RAD 6 | 1.0530157 | 0.9691597 | 0.9040673 | 0.8820972 | 0.9763135 | 0.9764624 | 1.6117871 |
| homologue) | | | | | | | |
| Phase-1 RCT-173 | 1.2212738 | 0.9523045 | 1.1816298 | 1.0549021 | 0.9468988 | 0.9994228 | 1.8045489 |
| Ornithine decarboxylase | 1.1954589 | 1.0377861 | 1.1071548 | 1.2378452 | 1.1849977 | 1.2795186 | 2.7529294 |
| Zinc finger protein | 1.0401613 | 0.9787151 | 1.1120123 | 1.0592479 | 1.0887815 | 0.9989316 | 2.640505 |
| CDK108 | 0.9289708 | 1.1418519 | 1.3328434 | 1.1719767 | 0.6955004 | 0.8719931 | 0.8136677 |
| Phase-1 RCT-117 | 0.7949771 | 0.975597 | 0.9532428 | 0.9088209 | 0.6411522 | 1.1965982 | 0.6033082 |
| {Phase-1 RCT 98} | 0.9450135 | 0.9875153 | 0.9556741 | 0.8696497 | 1.0220274 | 0.8827683 | 0.6566961 |
| Aquaporin-3 (AQP3) | 0.9138117 | 0.9036757 | 1.0496352 | 0.9408348 | 1.0217019 | 0.9605978 | 0.7852445 |
| Cholesterol esterase | 0.8608466 | 0.657684 | 0.8786566 | 0.8666829 | 0.7698643 | 0.7699159 | 0.2323421 |
| Ribosomal protein S17 | 0.8424332 | 0.821301 | 0.8002396 | 0.7998509 | 0.8217851 | 0.7943193 | 2.8501992 |
| Poly(ADP-ribose) polymerase | 1.0492022 | 0.9449236 | 0.8881267 | 1.0632423 | 0.9187188 | 0.9485507 | 1.8375245 |
| Annexin V | 0.9960867 | 0.782895 | 1.0344614 | 0.9270964 | 1.0183861 | 1.1806387 | 3.7135267 |
| Phase-1 RCT-33 | 1.0513141 | 1.0208912 | 0.8650795 | 0.9665988 | 1.1613983 | 1.1954288 | 0.2595179 |
| Phase-1 RCT-61 | 1.017836 | 0.9157198 | 0.8684428 | 1.0402333 | 0.9279007 | 0.9786679 | 1.4505388 |
| Retinol dehydrogenase type III | 1.00604 | 1.1517749 | 0.9983751 | 1.0194402 | 1.0130548 | 1.1555505 | 0.1848336 |
| ATPase inhibitor (rat mitochondrial IF1 protein) | 0.5725072 | 0.729885 | 0.663565 | 0.6411699 | 0.7143095 | 0.6913182 | 0.1009984 |
| Thymidylate synthase | 1.0896528 | 0.9540239 | 1.3407125 | 1.1046199 | 1.4454896 | 1.348726 | 1.083676 |
| Interleukin-18 | 0.9687101 | 0.9214739 | 1.3595958 | 1.1744161 | 1.4215939 | 1.4431158 | 4.5706115 |
| Lecithin: cholesterol acyltransferase | 1.0029972 | 1.0301572 | 0.8950343 | 1.0578127 | 1.2629851 | 1.0963795 | 0.3279749 |
| Contrapsin-like protease inhibitor (CPi-21) | 0.7812051 | 0.7477906 | 1.0113498 | 0.8961986 | 1.1519202 | 1.1516407 | 0.3400995 |
| Proliferating cell nuclear antigen gene | 1.0201807 | 0.8158989 | 1.1410704 | 1.0238171 | 0.8775057 | 0.9469696 | 1.0752137 |
| Phase-1 RCT-230 | 1.0366474 | 1.2087572 | 1.11468 | 1.1576287 | 0.9696152 | 2.5295217 | 3.2487206 |
| Cytochrome P450 2D18 | 1.1358376 | 1.1605132 | 1.0560999 | 1.0338767 | 1.1123867 | 0.9066766 | 0.319027 |
| Phase-1 RCT-48 | 0.9506919 | 1.6124053 | 1.0106314 | 0.7709962 | 0.992533 | 0.7230961 | 0.5559517 |
| Phase-1 RCT-292 | 0.906597 | 1.0188742 | 0.8766699 | 0.9129641 | 0.9965693 | 0.8973333 | 0.9722806 |
| Arginosuccinate synthetase 1 | 0.8495073 | 1.7028462 | 0.8816573 | 1.0471342 | 0.9115225 | 0.8125272 | 0.3783045 |
| C-reactive protein | 0.8468158 | 1.2573138 | 0.8438098 | 1.0362647 | 1.0968858 | 1.0183805 | 0.1968424 |

| | AFLB | AFLB | AMPB | AMPB | AMPB | AMPB |
|---|---|---|---|---|---|---|
| Compound-Dose | 1 | 1 | 5 | 5 | 5 | 20 |
| Animal Number | 155 | 156 | 447 | 448 | 449 | 457 |
| Liver Toxicity Necrosis Classification | yes | yes | no | no | no | no |
| Gene Name | | | | | | |
| Osteoactivin | 0.8615244 | 1.0317463 | 0.9125878 | 1.1674411 | 0.8973806 | 1.2225242 |
| Stathmin | 0.9633835 | 1.0783936 | 1.250439 | 1.0094224 | 1.3013003 | 1.0451556 |
| Calpactin I heavy chain | 0.9675314 | 1.023595 | 1.1163723 | 0.910305 | 1.0837501 | 1.1460541 |
| Phase-1 RCT-179 | 1.1845527 | 1.0558717 | 0.8107591 | 0.9831401 | 0.9515417 | 1.1169256 |
| Phase-1 RCT-107 | 0.7435929 | 0.7626921 | 1.1841815 | 1.1149586 | 1.0991063 | 0.9410233 |
| Heme oxygenase | 0.892726 | 0.8316786 | 0.7873155 | 1.137639 | 1.0931028 | 1.3451879 |
| Collagen type II | 0.9134976 | 1.0346814 | 1.1569868 | 0.9924081 | 0.9031389 | 0.9455115 |
| Phase-1 RCT-81 | 0.925662 | 1.0240374 | 0.923279 | 1.1157246 | 0.8628684 | 0.9211569 |
| Glycine methyltransferase | 0.5086108 | 0.5080552 | 0.8564665 | 1.0037373 | 0.8129624 | 0.9203792 |
| Cystatin C | 1.0397563 | 0.962675 | 0.791621 | 0.8461584 | 0.5761641 | 1.2188829 |
| Melanoma-associated antigen ME491 | 1.0747501 | 0.9253304 | 1.4041648 | 1.0117372 | 1.176277 | 1.0263802 |
| Phase-1 RCT-146 | 0.9794613 | 1.0101681 | 1.696403 | 1.1328968 | 1.2406712 | 1.0565294 |
| Gamma-actin, cytoplasmic | 0.8693094 | 1.2397884 | 0.7553826 | 0.7559063 | 0.5924193 | 0.8748369 |
| Adenine nucleotide translocator 1 | 1.4194974 | 1.058713 | 0.9155044 | 0.9238917 | 0.9791834 | 0.8268881 |
| Thymosin beta-10 | 0.8672957 | 1.0802332 | 0.795069 | 0.906053 | 0.6954628 | 1.5268857 |
| Insulin-like growth factor I, exon 6 | 1.2470008 | 1.0672983 | 0.6142175 | 1.0356677 | 0.9111454 | 0.9183138 |
| Phase-1 RCT-78 | 1.0170801 | 0.9852047 | 0.959868 | 0.797551 | 0.9696478 | 1.0339892 |
| Alpha-2-microglobulin | 1.1356126 | 0.5337375 | 0.5222589 | 0.8431013 | 0.3137171 | 0.9376595 |
| High affinity IgE receptor gamma chain | 0.9322605 | 0.9114644 | 0.983659 | 0.9042296 | 0.9116559 | 1.385185 |
| (FcERIgamma) | | | | | | |
| 60S ribosomal protein L6 | 1.2841421 | 1.0570842 | 0.8431895 | 1.171523 | 0.9318588 | 1.0142978 |
| IgE binding protein | 0.9509467 | 0.8918181 | 1.281797 | 1.0479465 | 1.1409715 | 1.3118135 |
| Phase-1 RCT-149 | 0.9192969 | 0.8770192 | 1.103985 | 1.0361313 | 1.0488902 | 1.000186 |
| Cytochrome P450 2C11 | 0.9914832 | 0.5426267 | 0.9910386 | 1.117169 | 0.7460099 | 1.2184696 |
| Uncoupling protein 2 | 0.8327372 | 0.8698462 | 0.9577513 | 0.9801776 | 1.1875461 | 1.4645445 |
| Cofilin | 1.1576402 | 1.0285174 | 0.5966381 | 0.729577 | 0.5394255 | 0.7657474 |
| Alpha-tubulin | 1.1990678 | 1.1889961 | 1.187194 | 1.0309474 | 1.304522 | 0.9840585 |
| Stearyl-CoA desaturase, liver | 0.6385864 | 0.9496064 | 0.4948963 | 0.4780226 | 0.5050477 | 0.2426843 |
| Glutathione S-transferase theta-1 | 1.2043787 | 1.0252804 | 0.9979312 | 0.9510303 | 0.7525199 | 0.9824373 |
| alpha-1,2-fucosyltrans-ferase | 1.0526489 | 1.036814 | 0.6724361 | 0.8342459 | 0.7873911 | 1.2615771 |
| Phase-1 RCT-138 | 0.9415078 | 0.9440446 | 1.084472 | 0.9515162 | 1.0480969 | 1.1863898 |
| Phase-1 RCT-295 | 1.0815469 | 1.1372379 | 0.8970316 | 0.9094898 | 0.9478506 | 1.0633602 |
| Phase-1 RCT-164 | 1.0466295 | 0.9626465 | 0.8687149 | 0.84719 | 0.7584506 | 0.976139 |
| Alpha-2-macroglobulin, sequence 2 | 0.8776702 | 1.0753059 | 0.9067587 | 0.830195 | 0.6386585 | 0.8815583 |
| Biliverdin reductase | 0.779675 | 0.9543671 | 1.1425332 | 0.9936371 | 1.0002049 | 1.1934937 |
| Phase-1 RCT-121 | 0.8713264 | 0.9568241 | 1.3244894 | 1.0861709 | 1.337051 | 1.2118112 |
| Dynamin-1 (D100) | 0.9949011 | 0.9873377 | 0.7778178 | 0.8284993 | 0.9892056 | 0.8990399 |
| Phase-1 RCT-192 | 1.2777597 | 1.1319622 | 0.7102678 | 0.8280568 | 1.0700417 | 1.1617124 |
| Fatty acid synthase | 0.7436091 | 1.2139999 | 0.8345588 | 0.7200248 | 0.7470871 | 0.5696161 |
| Phase-1 RCT-211 | 1.0214303 | 1.0741385 | 1.1609129 | 0.9068038 | 1.0667796 | 1.1268951 |
| Ribosomal protein S9 | 0.9514397 | 0.8761662 | 0.7072545 | 0.9098741 | 0.5763723 | 0.8896182 |
| NADP-dependent isocitrate dehydrogenase, | 1.2675751 | 1.1159633 | 0.7439401 | 0.8456152 | 0.773966 | 0.9667085 |
| cytosolic | | | | | | |
| Phase-1 RCT-57 | 1.0851213 | 1.0211517 | 0.7734788 | 0.7652482 | 0.9953002 | 0.9424693 |
| Phase-1 RCT-36 | 0.9003484 | 0.9890391 | 1.1249701 | 0.9816362 | 1.121261 | 1.0431013 |
| Apolipoprotein All | 0.7052455 | 0.681849 | 0.4971535 | 0.8976258 | 0.5233711 | 1.3205215 |
| Phase-1 RCT-68 | 1.0487605 | 1.0038353 | 1.2638565 | 0.9616104 | 1.0947447 | 1.0248705 |
| Multidrug resistant protein-2 | 0.9825953 | 1.1341027 | 1.5056955 | 1.1442175 | 1.0736461 | 1.121198 |
| Phase-1 RCT-39 | 1.0271404 | 1.0339537 | 1.8241241 | 1.1903906 | 1.3744453 | 1.1361084 |
| NIPK | 0.9394025 | 1.0780785 | 1.1850382 | 1.0815257 | 1.137849 | 0.957609 |
| F1-ATPase beta subunit | 1.3208119 | 1.0628144 | 0.6900291 | 0.885393 | 0.5929795 | 0.7621051 |
| Elongation factor-1 alpha | 1.011157 | 1.0898534 | 0.5979561 | 0.781841 | 0.5395579 | 0.9502931 |
| Phase-1 RCT-24 | 1.0907329 | 1.1682153 | 1.0196615 | 0.86361 | 1.0654734 | 0.9007047 |
| Beta-tubulin, class I | 0.9557724 | 1.1232898 | 0.800837 | 0.6437594 | 0.8968343 | 0.8906808 |
| 14-3-3 zeta | 1.1339043 | 1.1298517 | 1.2500001 | 1.1097312 | 1.0639106 | 0.9552526 |
| Alpha-prothymosin | 1.0955477 | 1.0402584 | 0.5142798 | 0.7254174 | 0.5318328 | 0.9342166 |
| Phase-1 RCT-58 | 1.0852432 | 1.0231446 | 0.95126 | 0.9795181 | 0.7161284 | 1.7438189 |
| Phase-1 RCT-290 | 0.697883 | 0.7904937 | 0.8673889 | 0.7637988 | 0.8829273 | 1.0126884 |
| Transitional endoplasmic reticulum ATPase | 1.1518599 | 1.0644834 | 1.1483935 | 1.1571354 | 1.1540842 | 0.8053178 |
| Beta-actin | 0.5492648 | 1.0294207 | 0.7983956 | 0.7948111 | 0.5783896 | 1.073051 |
| Thioredoxin-1 (Trx1) | 1.1632018 | 0.8763781 | 0.5787691 | 0.8391015 | 0.630973 | 1.2275441 |
| Phase-1 RCT-174 | 0.9950138 | 0.967585 | 0.7588874 | 0.8038466 | 0.8482606 | 0.8092277 |
| Beta-actin, sequence 2 | 1.0929923 | 1.0866972 | 0.697978 | 0.7247665 | 0.714939 | 1.2761545 |
| Phase-1 RCT-109 | 0.8838193 | 1.0956271 | 0.8342056 | 0.8363367 | 0.9605117 | 1.0944417 |
| Phase-1 RCT-154 | 1.828655 | 1.2494447 | 1.2052535 | 0.9005436 | 1.1758225 | 1.0363064 |
| Hypoxanthine-guanine | 1.0269508 | 0.970857 | 0.7845859 | 0.7378945 | 0.7687168 | 0.9893281 |
| phosphoribosyltransferase | | | | | | |
| Phase-1 RCT-213 | 1.0894368 | 1.101493 | 1.1562724 | 1.0522703 | 1.23028 | 0.9288387 |
| Phase-1 RCT-260 | 0.9091258 | 0.9539264 | 2.0722747 | 1.2264636 | 1.4855573 | 1.0473646 |
| Phase-1 RCT-12 | 0.8693203 | 1.0272548 | 1.0543516 | 0.8187002 | 0.9648743 | 0.9497279 |
| Phase-1 RCT-293 | 0.4534271 | 0.9803152 | 0.9957148 | 1.0942656 | 0.9148405 | 1.3929162 |
| Ribosomal protein S8 | 1.5105716 | 1.1729922 | 0.5201059 | 0.6801564 | 0.5445867 | 1.1288764 |
| Pyruvate kinase, muscle | 0.8323526 | 1.0273856 | 1.3680781 | 1.0077512 | 1.2806172 | 1.1181059 |
| Nucleoside diphosphate kinase beta isoform | 0.9441696 | 1.0231937 | 0.7493784 | 0.7724961 | 0.7195505 | 0.9240763 |
| p55CDC | 1.0612808 | 1.0325192 | 1.6387985 | 1.207202 | 1.3222799 | 1.018106 |
| Phase-1 RCT-156 | 1.0160891 | 1.0384649 | 0.9112378 | 0.9658345 | 0.8954074 | 0.8132399 |
| Ribosomal protein L13A | 0.6950438 | 0.9571143 | 0.6829917 | 0.8198752 | 0.6252306 | 1.3499919 |
| Phase-1 RCT-258 | 1.0731311 | 1.0555085 | 1.2589285 | 1.1672269 | 1.4235071 | 1.0165482 |
| Insulin-like growth factor I | 1.22298 | 0.9970888 | 0.5236766 | 0.8385667 | 0.5344141 | 0.798738 |
| Cytochrome P450 3A1 | 1.4397556 | 0.7336742 | 0.5147288 | 0.9032412 | 0.4489328 | 0.9419069 |
| {Ribosomal protein 16} | 1.2285621 | 1.090688 | 0.674931 | 0.8326754 | 0.7612244 | 0.9671924 |
| Organic cation transporter 3 | 1.279594 | 1.0502512 | 0.7784874 | 1.1630278 | 1.2574313 | 0.981805 |
| Calpain 2 | 1.0434009 | 1.0320668 | 1.390484 | 1.0323693 | 1.0606524 | 0.9459109 |
| Phase-1 RCT-102 | 1.1656674 | 0.8150439 | 1.1480837 | 0.9434534 | 0.9515201 | 0.7971229 |
| Equilbrative nitrobenzylthioinosine-sensitive | 1.060696 | 0.8971971 | 1.0199871 | 1.0683601 | 0.7600734 | 0.9563016 |
| nucleoside transporter | | | | | | |
| Phase-1 RCT-227 | 1.2109516 | 0.9574384 | 0.7488204 | 0.7322165 | 0.762748 | 1.021454 |
| N-hydroxy-2-acetylaminofluorene sulfotransferase (ST1C1) | 1.174153 | 0.7940673 | 0.7298397 | 0.9891685 | 0.5222511 | 0.9502388 |
| Cathepsin S | 0.8328223 | 1.0188015 | 1.1695855 | 1.1119341 | 0.9591986 | 1.4763538 |
| Apolipoprotein CIII | 0.9352753 | 0.8850537 | 0.9246666 | 1.0221066 | 0.8316063 | 0.9541129 |
| Cytochrome P450 2A3 | 0.9982439 | 1.136342 | 1.4082192 | 1.13151 | 1.1591414 | 0.6618254 |
| Cytochrome P450 2C23 | 1.0995017 | 0.9126788 | 0.6649759 | 0.7205547 | 0.5233288 | 0.7314729 |
| Apolipoprotein C1 | 1.0676221 | 0.6129826 | 0.5364373 | 0.5670971 | 0.4154754 | 0.6193115 |
| Betaine homocysteine methyltransferase (BHMT) | 0.7825452 | 0.6581869 | 0.6279419 | 0.6464171 | 0.7125044 | 1.1136348 |
| Paraoxonase 1 | 1.4374983 | 1.1509929 | 0.7416484 | 1.3832433 | 0.5291144 | 0.7538263 |
| Phase-1 RCT-207 | 1.9436535 | 1.3676472 | 1.436187 | 1.1076835 | 1.410432 | 1.068741 |
| Cathepsin B | 0.9621555 | 1.0201613 | 0.700828 | 0.9764546 | 0.7377672 | 1.031088 |
| Phase-1 RCT-144 | 0.9634991 | 1.0041808 | 1.3824978 | 1.1391203 | 1.187885 | 0.8863427 |
| Alpha-1 microglobulin/bikunin precursor (Ambp) | 1.3825171 | 1.040016 | 0.7834929 | 1.0410104 | 0.7050246 | 0.8547773 |
| Cyclin D1 | 1.2443913 | 1.0637841 | 1.1397063 | 1.3300958 | 0.983514 | 0.7904937 |
| Presenilin-1 | 1.140016 | 1.003872 | 0.5663167 | 1.1957431 | 0.5775192 | 0.6095333 |
| Protein tyrosine phosphatase, receptor type, D | 1.283737 | 0.9790217 | 0.7088335 | 0.8759474 | 0.6400611 | 1.0258703 |
| Multidrug resistant protein-1 | 1.4502778 | 1.2420723 | 0.9042078 | 0.9189555 | 1.0006263 | 1.0826333 |
| Cathepsin L, sequence 2 | 0.8830947 | 1.075219 | 0.9630337 | 0.923663 | 0.7258109 | 1.0112901 |
| JNK1 stress activated protein kinase | 0.9406804 | 1.0185857 | 1.4866103 | 1.141679 | 1.1607058 | 1.2968286 |
| Phase-1 RCT-43 | 0.8747907 | 0.9825339 | 0.9575816 | 0.9732381 | 0.9235079 | 0.8687258 |
| Tissue plasminogen activator | 0.967519 | 0.9546615 | 0.7174415 | 0.8003103 | 0.9446138 | 0.9229753 |
| Ubiquitin conjugating enzyme (RAD 6 | 1.3771667 | 1.0200104 | 0.8730195 | 1.011363 | 0.9513335 | 0.900085 |
| homologue) | | | | | | |
| Phase-1 RCT-173 | 0.8803344 | 1.055568 | 1.4053158 | 1.0787874 | 1.2786305 | 0.9803516 |
| Ornithine decarboxylase | 1.0392871 | 1.1374121 | 0.7630888 | 0.5413718 | 1.0588022 | 0.8761777 |
| Zinc finger protein | 0.9683365 | 1.0279423 | 1.0118068 | 1.082599 | 1.0153428 | 0.9829586 |
| CDK108 | 0.8935317 | 0.8771762 | 1.2763253 | 0.9414818 | 1.0852044 | 0.9023485 |
| Phase-1 RCT-117 | 0.8603073 | 0.9173284 | 0.7810333 | 0.991488 | 1.0059144 | 1.2872521 |
| {Phase-1 RCT 98} | 0.9899724 | 1.0200299 | 1.236478 | 1.0820506 | 1.027432 | 0.818983 |
| Aquaporin-3 (AQP3) | 0.9485655 | 0.9437774 | 1.4833888 | 1.0456846 | 1.0865897 | 0.9164439 |
| Cholesterol esterase | 1.6882516 | 1.1603326 | 0.5649027 | 0.7092658 | 0.5896284 | 0.8474324 |
| Ribosomal protein S17 | 1.3345183 | 1.1354333 | 0.5489339 | 0.6880912 | 0.5094007 | 0.9039568 |
| Poly(ADP-ribose) polymerase | 0.9696912 | 1.0564777 | 1.2958622 | 1.0225128 | 1.2818465 | 1.0500612 |
| Annexin V | 1.1681834 | 1.0432401 | 1 | 0.9228605 | 1.1789558 | 0.9516839 |
| Phase-1 RCT-33 | 1.368556 | 1.183645 | 0.8002285 | 0.8656405 | 0.9782263 | 1.2397981 |
| Phase-1 RCT-61 | 1.0322316 | 1.0532357 | 1.2904439 | 1.0639186 | 1.2913243 | 1.0595719 |
| Retinol dehydrogenase type III | 1.2205725 | 0.9278845 | 0.5662104 | 0.6876118 | 0.5020449 | 0.9959416 |
| ATPase inhibitor (rat mitochondrial IF1 protein) | 1.3426714 | 0.8957921 | 0.3448964 | 0.7311564 | 0.3106828 | 1.0149113 |
| Thymidylate synthase | 1.1451863 | 0.9950822 | 1.7977358 | 1.3181409 | 1.4102528 | 1.0841591 |
| Interleukin-18 | 1.0428894 | 0.9738743 | 1.9853352 | 1.2988793 | 1.5210843 | 1.1517737 |
| Lecithin: cholesterol acyltransferase | 1.0398725 | 1.0353965 | 0.6228899 | 0.9354507 | 0.7145761 | 1.1522353 |
| Contrapsin-like protease inhibitor (CPi-21) | 1.0106711 | 0.9019488 | 0.4391919 | 0.5263366 | 0.5464051 | 0.7735558 |
| Proliferating cell nuclear antigen gene | 0.7641678 | 0.9438332 | 1.4332315 | 1.1575084 | 1.1337144 | 1.1177591 |
| Phase-1 RCT-230 | 0.8196799 | 0.9355146 | 1.9163396 | 1.1768075 | 1.4005544 | 1.2182211 |
| Cytochrome P450 2D18 | 1.0204064 | 0.997489 | 0.5771297 | 0.5775753 | 0.7153438 | 0.948432 |
| Phase-1 RCT-48 | 0.982266 | 0.7924252 | 0.6943718 | 0.7623019 | 0.885537 | 1.0376565 |
| Phase-1 RCT-292 | 0.8925946 | 0.8941038 | 1.1609571 | 1.0046117 | 1.0895233 | 0.8930761 |
| Arginosuccinate synthetase 1 | 0.7569478 | 0.8988513 | 0.6747679 | 0.6030883 | 0.7680519 | 0.6030882 |
| C-reactive protein | 1.1719352 | 0.9674087 | 0.7386256 | 1.1804248 | 0.8218448 | 1.253389 |

| | AMPB | AMPB | ANIT | ANIT | ANIT | ANIT | ANIT |
|---|---|---|---|---|---|---|---|
| Compound-Dose | 20 | 20 | 15 | 15 | 15 | 60 | 60 |
| Animal Number | 458 | 459 | 1647 | 1648 | 1649 | 1657 | 1658 |
| Liver Toxicity Necrosis Classification | no | no | no | no | no | yes | yes |
| Gene Name | | | | | | | |
| Osteoactivin | 1.056018 | 1.0182256 | 1.0333582 | 0.8825936 | 0.9014688 | 3.4732332 | 5.9562383 |
| Stathmin | 1.0147207 | 0.9429878 | 0.9824678 | 0.9931266 | 0.99955 | 2.2205188 | 2.7410111 |
| Calpactin I heavy chain | 1.1426637 | 1.0306933 | 1.2041775 | 1.1720455 | 1.1183621 | 1.9681658 | 3.448336 |
| Phase-1 RCT-179 | 1.32218 | 1.1325254 | 0.8557684 | 0.902192 | 0.9304127 | 1.1078522 | 1.540269 |
| Phase-1 RCT-107 | 0.9202452 | 1.0063459 | 1.4503918 | 1.0692983 | 1.1789786 | 0.7279947 | 0.6111192 |
| Heme oxygenase | 1.4164733 | 1.6153797 | 1.0255673 | 1.2719535 | 0.8498396 | 2.1185217 | 2.098342 |
| Collagen type II | 1.0408648 | 0.8488649 | 1.2868096 | 1.1749486 | 1.088599 | 1.0222864 | 1.1164658 |
| Phase-1 RCT-81 | 0.9497643 | 0.9520583 | 0.9323599 | 1.0607305 | 1.067143 | 0.6070271 | 0.6778485 |
| Glycine methyltransferase | 0.7441285 | 0.9673389 | 1.6287463 | 1.0565147 | 1.3327351 | 0.1747613 | 0.1511171 |
| Cystatin C | 1.0016186 | 1.0835875 | 0.7726788 | 0.965853 | 0.8330676 | 0.8599725 | 1.135395 |
| Melanoma-associated antigen ME491 | 1.1435308 | 1.036014 | 1.0971026 | 1.116065 | 1.3212427 | 1.8764677 | 2.6273792 |
| Phase-1 RCT-146 | 1.1138287 | 1.0177294 | 1.0696093 | 0.9992808 | 0.7708949 | 2.546177 | 3.0612671 |
| Gamma-actin, cytoplasmic | 0.7037224 | 0.732093 | 1.12372 | 0.8992598 | 0.9000002 | 2.6424177 | 3.0877373 |
| Adenine nucleotide translocator 1 | 0.949501 | 0.8913757 | 0.8854926 | 0.9897101 | 0.9647229 | 1.3702029 | 1.6169317 |
| Thymosin beta-10 | 0.9862257 | 1.0101712 | 0.9350083 | 0.9479762 | 0.9169012 | 3.005312 | 3.6569297 |
| Insulin-like growth factor I, exon 6 | 0.7381311 | 0.8743687 | 0.8777093 | 0.6808231 | 1.10884 | 0.3061498 | 0.3694823 |
| Phase-1 RCT-78 | 0.9280838 | 2.089191 | 1.0058997 | 1.0411944 | 1.1049261 | 0.7175146 | 0.6992037 |
| Alpha-2-microglobulin | 0.57723 | 0.6076822 | 0.5998429 | 0.8970993 | 1.0269518 | 0.1600336 | 0.1087527 |
| High affinity IgE receptor gamma chain | 0.9995303 | 1.0533898 | 0.9418104 | 0.9372602 | 0.8409146 | 1.2047417 | 1.7143995 |
| (FcERIgamma) | | | | | | | |
| 60S ribosomal protein L6 | 0.9798036 | 0.9403514 | 0.8858253 | 0.9773269 | 0.9604464 | 1.3861839 | 1.6374292 |
| IgE binding protein | 1.0924672 | 1.0562913 | 1.0813518 | 1.0035636 | 0.9304777 | 2.9692059 | 4.100765 |
| Phase-1 RCT-149 | 1.0625994 | 0.9958697 | 0.9586629 | 0.9036391 | 0.9488998 | 0.9342293 | 0.8402072 |
| Cytochrome P450 2C11 | 1.1133205 | 1.2780035 | 0.9477041 | 0.9752713 | 0.9150951 | 0.9656566 | 0.9482312 |
| Uncoupling protein 2 | 1.066116 | 0.9593831 | 1.0745637 | 1.0177927 | 0.9284039 | 1.9237328 | 2.916121 |
| Cofilin | 0.8179917 | 0.774172 | 0.9546309 | 0.9982426 | 1.0498123 | 1.8022687 | 2.279488 |
| Alpha-tubulin | 1.0206833 | 0.9683974 | 1.3282887 | 1.1946946 | 1.1954165 | 6.1806784 | 5.144883 |
| Stearyl-CoA desaturase, liver | 0.4474725 | 0.7099159 | 0.4101313 | 0.4246849 | 0.5435675 | 0.6488332 | 0.4966976 |
| Glutathione S-transferase theta-1 | 1.0051966 | 1.0397239 | 0.8306566 | 0.8896161 | 0.9610965 | 0.8316869 | 0.7689509 |
| alpha-1,2-fucosyltrans-ferase | 0.9823999 | 1.0619117 | 0.8127688 | 0.8394252 | 0.7734017 | 0.8446521 | 0.7608296 |
| Phase-1 RCT-138 | 0.95427 | 0.9967405 | 0.9930451 | 0.9192971 | 0.9106892 | 1.0609335 | 1.4357718 |
| Phase-1 RCT-295 | 0.9750138 | 1.0769008 | 0.8728036 | 0.9919838 | 0.9769249 | 1.5787644 | 1.8926668 |
| Phase-1 RCT-164 | 0.9590212 | 0.851925 | 1.0772586 | 1.0384443 | 0.9796284 | 0.7339255 | 0.5855588 |
| Alpha-2-macroglobulin, sequence 2 | 0.8541158 | 1.0311315 | 0.9534459 | 0.9376854 | 0.8819852 | 1.1932036 | 1.2979295 |
| Biliverdin reductase | 1.170278 | 1.1163765 | 1.0736909 | 1.1431781 | 1.0300943 | 1.5669832 | 2.1722279 |
| Phase-1 RCT-121 | 1.0916413 | 0.9583515 | 1.1452204 | 1.0730885 | 0.9466611 | 2.1528463 | 1.9514631 |
| Dynamin-1 (D100) | 1.0375509 | 0.9539098 | 0.7863509 | 0.8118657 | 1.054597 | 0.656271 | 0.4042521 |
| Phase-1 RCT-192 | 0.9347814 | 0.964452 | 0.9940069 | 0.967965 | 0.9027362 | 1.309023 | 1.5078654 |
| Fatty acid synthase | 0.8619639 | 1.1670654 | 0.4587784 | 0.646252 | 0.3625394 | 0.7054457 | 0.587433 |
| Phase-1 RCT-211 | 0.4435457 | 1.1001171 | 0.9730041 | 0.8706321 | 0.9523888 | 2.1073022 | 2.519016 |
| Ribosomal protein S9 | 0.97504 | 0.9107216 | 0.9966961 | 0.9806838 | 1.012105 | 1.6674106 | 1.7230276 |
| NADP-dependent isocitrate dehydrogenase, | 0.8761427 | 0.89822 | 0.8235987 | 1.0444142 | 0.949084 | 0.7798426 | 0.9359724 |
| cytosolic | | | | | | | |
| Phase-1 RCT-57 | 0.8642175 | 0.7461707 | 1.0717546 | 1.1878927 | 1.113291 | 0.7661114 | 0.8393809 |
| Phase-1 RCT-36 | 1.0295292 | 0.9883316 | 0.913987 | 0.9825187 | 0.9876536 | 1.0161265 | 0.7709469 |
| Apolipoprotein All | 0.8051868 | 1.0551484 | 1.3969821 | 1.1068813 | 1.0674278 | 0.5663756 | 0.33596 |
| Phase-1 RCT-68 | 1.0319358 | 0.9511939 | 1.0169325 | 1.1429626 | 1.0403204 | 1.4898045 | 1.848668 |
| Multidrug resistant protein-2 | 1.07752 | 1.1496143 | 1.1874845 | 1.1534436 | 1.0897099 | 6.224701 | 7.882569 |
| Phase-1 RCT-39 | 1.1341487 | 1.128192 | 1.0224286 | 1.057367 | 0.9790058 | 1.2794921 | 1.53414 |
| NIPK | 0.9723628 | 0.9261184 | 0.9981779 | 0.9193227 | 1.0082444 | 1.0858938 | 1.0181683 |
| F1-ATPase beta subunit | 0.7238271 | 0.8626145 | 0.8795612 | 0.9641006 | 1.0957649 | 0.6991774 | 0.938277 |
| Elongation factor-1 alpha | 0.7167143 | 0.8739466 | 0.8943721 | 0.9262248 | 0.9615979 | 0.8750632 | 1.1521223 |
| Phase-1 RCT-24 | 0.8607509 | 0.8075434 | 1.2047797 | 1.032105 | 1.0418203 | 1.1332814 | 1.4239485 |
| Beta-tubulin, class I | 0.7363347 | 0.7157833 | 1.0639292 | 0.9390429 | 1.0123998 | 6.9426236 | 7.5364776 |
| 14-3-3 zeta | 1.1251712 | 0.9639939 | 1.0534065 | 1.094678 | 1.29418 | 1.880126 | 2.4726498 |
| Alpha-prothymosin | 0.8937961 | 0.8630645 | 0.6845507 | 0.8342262 | 0.8183244 | 1.1669241 | 1.6442423 |
| Phase-1 RCT-58 | 0.9279539 | 0.867276 | 1.2292962 | 0.7624725 | 1.1427739 | 1.1259775 | 1.1986583 |
| Phase-1 RCT-290 | 1.0842041 | 1.1947466 | 1.0829717 | 0.819415 | 1.1768513 | 0.5175455 | 0.5300809 |
| Transitional endoplasmic reticulum ATPase | 0.9232877 | 0.8329106 | 1.2360516 | 1.3623552 | 1.3094363 | 0.9498323 | 1.1979697 |
| Beta-actin | 0.6349538 | 0.7142041 | 1.1888083 | 0.8811165 | 0.9876559 | 1.7448947 | 1.9209703 |
| Thioredoxin-1 (Trx1) | 0.9995525 | 1.0733508 | 0.798615 | 0.8657769 | 0.8407176 | 1.1920848 | 1.467968 |
| Phase-1 RCT-174 | 0.7657149 | 0.8592543 | 0.8866135 | 1.0016433 | 1.0119494 | 0.7712758 | 0.8763287 |
| Beta-actin, sequence 2 | 0.7607747 | 0.8335964 | 1.0828733 | 0.89847 | 1.0296172 | 1.6286896 | 2.001358 |
| Phase-1 RCT-109 | 0.8537247 | 0.9175308 | 0.9605108 | 0.983778 | 0.9494119 | 1.4613513 | 1.634832 |
| Phase-1 RCT-154 | 1.108054 | 0.9948319 | 0.9789994 | 0.9664544 | 0.9848525 | 1.6750766 | 2.358975 |
| Hypoxanthine-guanine | 0.9961924 | 0.7957512 | 0.8740243 | 1.014736 | 0.9731789 | 1.9589052 | 1.8787786 |
| phosphoribosyltransferase | | | | | | | |
| Phase-1 RCT-213 | 1.0030487 | 0.9003825 | 0.9811747 | 1.0646389 | 1.1104083 | 1.0350868 | 1.3180627 |
| Phase-1 RCT-260 | 1.010093 | 1.1170255 | 0.9985426 | 1.044127 | 0.9915931 | 0.6918958 | 0.7451444 |
| Phase-1 RCT-12 | 0.878989 | 0.8230778 | 1.040774 | 1.0051653 | 1.0406597 | 1.518563 | 1.5306286 |
| Phase-1 RCT-293 | 1.2597852 | 1.1168697 | 0.9834956 | 0.9353489 | 0.960715 | 1.5456666 | 2.118387 |
| Ribosomal protein S8 | 0.9248457 | 1.0085368 | 0.8009844 | 0.8433974 | 0.8371554 | 1.2650287 | 1.5453748 |
| Pyruvate kinase, muscle | 1.0366606 | 1.116298 | 1.1001874 | 1.0477198 | 1.0633588 | 1.5231359 | 2.500936 |
| Nucleoside diphosphate kinase beta isoform | 0.8192705 | 0.8706 | 1.040557 | 1.0243948 | 1.0360467 | 1.2355199 | 1.1468763 |
| p55CDC | 1.077894 | 1.0128747 | 1.1438805 | 1.0202894 | 0.9278312 | 5.9877205 | 4.510624 |
| Phase-1 RCT-156 | 0.817032 | 0.8678975 | 0.9477462 | 0.9565282 | 1.0615103 | 1.4165276 | 1.4924241 |
| Ribosomal protein L13A | 1.0324097 | 1.0203551 | 0.9332788 | 0.9380892 | 0.8876405 | 1.4593916 | 1.6768426 |
| Phase-1 RCT-258 | 1.0635455 | 0.9337896 | 1.0533118 | 1.1042937 | 1.156895 | 1.3947822 | 1.4659443 |
| Insulin-like growth factor I | 0.9076874 | 0.9642084 | 0.7033805 | 0.6339206 | 0.8773336 | 0.2898818 | 0.310505 |
| Cytochrome P450 3A1 | 0.6546494 | 0.9715524 | 0.6370456 | 0.8559843 | 1.1856766 | 0.2287413 | 0.1942196 |
| {Ribosomal protein 16} | 0.9227381 | 0.9613035 | 0.8017798 | 0.8621198 | 0.8689778 | 1.2038296 | 1.4534042 |
| Organic cation transporter 3 | 1.1017131 | 1 | 0.8995573 | 0.9599086 | 0.9490131 | 1.2909509 | 1.5530481 |
| Calpain 2 | 0.9790413 | 0.954222 | 0.9938647 | 0.9774466 | 0.9930515 | 1.381663 | 1.7618228 |
| Phase-1 RCT-102 | 0.841759 | 0.7601402 | 1.0093353 | 0.6505152 | 1.0963286 | 1.0339808 | 0.2965995 |
| Equilbrative nitrobenzylthioinosine-sensitive | 0.7170103 | 0.8633407 | 0.809563 | 0.919627 | 0.932206 | 0.7611795 | 0.6371979 |
| nucleoside transporter | | | | | | | |
| Phase-1 RCT-227 | 0.832433 | 0.9085563 | 0.7113149 | 0.8469383 | 1.0879065 | 0.2302121 | 0.2120681 |
| N-hydroxy-2-acetylaminofluorene sulfotransferase (ST1C1) | 0.8270136 | 0.9833823 | 0.6856463 | 0.8485147 | 0.8589159 | 0.3046154 | 0.4018374 |
| Cathepsin S | 1.1445665 | 1.0871803 | 1.1206547 | 1.2086037 | 0.9291799 | 1.22348 | 1.7622389 |
| Apolipoprotein CIII | 0.9099588 | 0.9745081 | 0.6908609 | 0.8645765 | 1.0026829 | 0.3362517 | 0.3710449 |
| Cytochrome P450 2A3 | 0.9009761 | 0.8446443 | 0.8733484 | 0.8943771 | 0.8824588 | 0.6240841 | 0.4891037 |
| Cytochrome P450 2C23 | 0.8186837 | 0.8084015 | 0.8794669 | 1.0112249 | 1.0878804 | 0.3707306 | 0.4201918 |
| Apolipoprotein C1 | 0.7237665 | 0.7232543 | 0.651229 | 0.7761082 | 0.8226616 | 0.2121816 | 0.2467121 |
| Betaine homocysteine methyltransferase (BHMT) | 1.0328261 | 1.3002588 | 1.1148337 | 0.759697 | 1.2334772 | 0.164473 | 0.1858653 |
| Paraoxonase 1 | 0.7265446 | 0.8428021 | 0.7563697 | 0.8475233 | 0.8718169 | 0.2522978 | 0.2769961 |
| Phase-1 RCT-207 | 1.1176119 | 0.968812 | 1.1584294 | 1.0129689 | 1.1058992 | 1.5422435 | 2.1614718 |
| Cathepsin B | 0.9697614 | 1.0331763 | 0.8750228 | 0.9008336 | 1.0638956 | 0.9409047 | 1.5576241 |
| Phase-1 RCT-144 | 1.1695511 | 0.957203 | 0.9754512 | 0.9052626 | 0.9560471 | 1.6237742 | 1.9486456 |
| Alpha-1 microglobulin/bikunin precursor (Ambp) | 0.8684684 | 0.8957668 | 0.8244814 | 1.0281191 | 1.0477533 | 0.3669841 | 0.4915671 |
| Cyclin D1 | 0.88927 | 0.9482615 | 0.8887642 | 0.9462849 | 1.0976832 | 3.935017 | 6.6928487 |
| Presenilin-1 | 0.6652092 | 0.6408032 | 0.6770415 | 0.8176213 | 1.0290071 | 0.24805 | 0.2819044 |
| Protein tyrosine phosphatase, receptor type, D | 0.9261665 | 1.0696032 | 0.7268044 | 0.696601 | 0.7478465 | 0.3627736 | 0.3354135 |
| Multidrug resistant protein-1 | 1.0119833 | 1.0792179 | 1.153148 | 1.1761672 | 1.0596014 | 5.848797 | 8.373859 |
| Cathepsin L, sequence 2 | 0.8886113 | 1.088411 | 0.925434 | 1.0204825 | 1.0711149 | 1.0810905 | 1.9204733 |
| JNK1 stress activated protein kinase | 1.10341 | 1.2580817 | 0.8087616 | 0.9187343 | 0.8178818 | 0.4208624 | 0.613389 |
| Phase-1 RCT-43 | 0.8600764 | 0.8153289 | 1.1794082 | 1.0199114 | 1.0554614 | 1.0733256 | 1.2281545 |
| Tissue plasminogen activator | 0.8835572 | 1.1318626 | 1.0078002 | 0.9908918 | 1.0128971 | 1.5977608 | 1.8362758 |
| Ubiquitin conjugating enzyme (RAD 6 | 1.057086 | 1.0294458 | 1.0568142 | 1.0751971 | 1.0558813 | 1.5829968 | 1.591105 |
| homologue) | | | | | | | |
| Phase-1 RCT-173 | 0.999857 | 0.7902179 | 1.195326 | 1.3948131 | 1.3588079 | 1.1845385 | 1.507371 |
| Ornithine decarboxylase | 0.7869446 | 0.7243339 | 1.103425 | 1.0479572 | 1.1045203 | 2.5105798 | 3.3767169 |
| Zinc finger protein | 1.0674223 | 0.9795439 | 1.1855369 | 1.0907849 | 1.1024027 | 2.377176 | 2.6799488 |
| CDK108 | 0.9214761 | 0.9610168 | 0.9546953 | 1.3245543 | 1.312092 | 0.8045728 | 0.9018864 |
| Phase-1 RCT-117 | 0.992003 | 1.3204676 | 0.9257302 | 1.1583569 | 1.3537414 | 0.7281377 | 0.6595961 |
| {Phase-1 RCT 98} | 1.0172758 | 0.8810394 | 1.0311644 | 0.9591566 | 1.0539962 | 0.9564288 | 0.7471356 |
| Aquaporin-3 (AQP3) | 1.0619748 | 1.0742525 | 0.9685384 | 0.9642112 | 1.2719256 | 1.047214 | 0.7458628 |
| Cholesterol esterase | 0.579205 | 0.5814209 | 0.7017274 | 1.2136436 | 1.0838976 | 0.1716152 | 0.3837978 |
| Ribosomal protein S17 | 0.711049 | 0.8855068 | 0.7384635 | 0.8393767 | 0.7736191 | 1.174896 | 1.5419097 |
| Poly(ADP-ribose) polymerase | 1.1880165 | 1.0103984 | 1.3619204 | 1.3036892 | 1.3005532 | 1.6234771 | 1.9413532 |
| Annexin V | 0.9684656 | 1.0033226 | 0.8561558 | 0.9727357 | 1.1157306 | 1.5704571 | 2.1399198 |
| Phase-1 RCT-33 | 0.9135903 | 1.0256609 | 0.7408863 | 0.9481106 | 0.9501658 | 0.7290353 | 0.6556532 |
| Phase-1 RCT-61 | 1.0555913 | 0.8530002 | 1.0120102 | 0.9591839 | 1.0589085 | 1.1785537 | 1.2905543 |
| Retinol dehydrogenase type III | 0.881651 | 0.8572318 | 0.8766584 | 1.0432407 | 0.9918767 | 0.3364937 | 0.3971592 |
| ATPase inhibitor (rat mitochondrial IF1 protein) | 0.9200221 | 0.9989137 | 0.6337205 | 0.7492762 | 0.7608547 | 0.2641971 | 0.2441437 |
| Thymidylate synthase | 1.1926887 | 1.1322815 | 1.0541902 | 0.9536973 | 0.9321356 | 2.2123704 | 1.8912121 |
| Interleukin-18 | 1.2613542 | 1.2034311 | 1.3960619 | 1.015742 | 0.9954116 | 1.2492509 | 1.3416272 |
| Lecithin: cholesterol acyltransferase | 0.9519377 | 0.9405515 | 0.9217619 | 0.9764004 | 1.1098969 | 0.4259491 | 0.4707979 |
| Contrapsin-like protease inhibitor (CPi-21) | 0.582172 | 0.7411926 | 0.7655159 | 0.9652755 | 0.7571916 | 0.2139093 | 0.2233552 |
| Proliferating cell nuclear antigen gene | 1.0603862 | 1.1330664 | 0.9878046 | 0.9421038 | 0.8697872 | 3.7038424 | 5.3185306 |
| Phase-1 RCT-230 | 1.0667944 | 1.064041 | 1.0760958 | 1.1071141 | 0.9634178 | 1.1565778 | 1.6861274 |
| Cytochrome P450 2D18 | 0.7058376 | 0.6476963 | 0.9772111 | 1.164403 | 1.063696 | 0.3546263 | 0.4245442 |
| Phase-1 RCT-48 | 0.920765 | 0.8640373 | 1.0831143 | 0.9469433 | 1.0532118 | 0.6145611 | 0.6610331 |
| Phase-1 RCT-292 | 1.0724427 | 0.9853155 | 1.0007368 | 0.9459087 | 1.0481787 | 0.7911491 | 0.7306105 |
| Arginosuccinate synthetase 1 | 0.8373551 | 0.8682028 | 0.8074041 | 1.052846 | 0.9390838 | 0.5797324 | 0.6516259 |
| C-reactive protein | 1.357493 | 1.3635825 | 0.7342775 | 0.6966915 | 0.7622907 | 0.2857386 | 0.3237806 |

| | ANIT | APAP | APAP | APAP | APAP | APAP |
|---|---|---|---|---|---|---|
| Compound-Dose | 60 | 250 | 250 | 250 | 1000 | 1000 |
| Animal Number | 1659 | 2127 | 2128 | 2129 | 2137 | 2138 |
| Liver Toxicity Necrosis Classification | yes | no | no | no | yes | yes |
| Gene Name | | | | | | |
| Osteoactivin | 1.1075405 | 0.9785781 | 0.9221771 | 1.0806009 | 0.9687991 | 1.2059599 |
| Stathmin | 1.0045274 | 0.9638528 | 1.0008512 | 0.9528105 | 1.1491044 | 0.9722596 |
| Calpactin I heavy chain | 1.1147871 | 1.2442296 | 1.334428 | 1.047676 | 1.2251767 | 1.0326775 |
| Phase-1 RCT-179 | 0.9463478 | 1.0460758 | 0.9856556 | 1.0572714 | 0.876736 | 1.1100013 |
| Phase-1 RCT-107 | 1.1908957 | 1.0908661 | 1.0513394 | 1.147697 | 1.3453178 | 1.4904277 |
| Heme oxygenase | 3.049268 | 1.1715741 | 1.1431412 | 1.1333971 | 1.5646038 | 1.5167644 |
| Collagen type II | 1.3440994 | 0.7774738 | 0.9185119 | 0.7931846 | 0.8804378 | 0.8396561 |
| Phase-1 RCT-81 | 0.9191542 | 0.9063141 | 0.9039519 | 0.9895008 | 0.8692108 | 0.8527127 |
| Glycine methyltransferase | 1.1584136 | 1.0155948 | 0.8681406 | 1.189958 | 1.4073533 | 1.7565162 |
| Cystatin C | 0.8870662 | 0.9326809 | 0.8226 | 1.1313648 | 0.9728723 | 0.9218378 |
| Melanoma-associated antigen ME491 | 1.2038822 | 0.9424909 | 0.7982191 | 0.994219 | 1.1597484 | 1.0475768 |
| Phase-1 RCT-146 | 1.1159716 | 1.0324131 | 1.100679 | 1.1208054 | 1.5817168 | 1.1772492 |
| Gamma-actin, cytoplasmic | 0.8200279 | 1.0082867 | 1.3434141 | 0.8083219 | 0.8285968 | 0.5686325 |
| Adenine nucleotide translocator 1 | 1.0412413 | 1.0163398 | 0.9829559 | 0.885921 | 0.8152595 | 0.8058795 |
| Thymosin beta-10 | 1.0618172 | 1.9914186 | 1.0010905 | 0.9217178 | 0.9289402 | 0.9721119 |
| Insulin-like growth factor I, exon 6 | 0.6322867 | 0.9416644 | 1.009605 | 1.2508193 | 0.7526835 | 0.742124 |
| Phase-1 RCT-78 | 1 | 0.9692544 | 0.9988523 | 1.0721903 | 1.1876335 | 0.8464135 |
| Alpha-2-microglobulin | 0.3131663 | 0.7555148 | 0.6541828 | 0.9808739 | 0.6332563 | 0.471282 |
| High affinity IgE receptor gamma chain | 1.9512392 | 0.9418423 | 0.9320222 | 0.9963262 | 0.8375078 | 0.8566253 |
| (FcERIgamma) | | | | | | |
| 60S ribosomal protein L6 | 0.9639581 | 1.1873882 | 1.1754264 | 0.9281063 | 0.8473364 | 0.8825963 |
| IgE binding protein | 0.9984356 | 1.1577263 | 1.1168134 | 1.0604401 | 1.1830835 | 1.1142046 |
| Phase-1 RCT-149 | 1.0186006 | 0.9639034 | 0.9524223 | 0.938849 | 1.0393528 | 0.9844413 |
| Cytochrome P450 2C11 | 0.9018759 | 0.9058629 | 0.8122948 | 0.841969 | 0.7517512 | 0.2760716 |
| Uncoupling protein 2 | 1.1284462 | 0.6871237 | 0.7920801 | 0.8637406 | 0.7321223 | 0.6932063 |
| Cofilin | 1.0114301 | 0.9977496 | 0.8981474 | 0.9858736 | 0.7770852 | 0.8352906 |
| Alpha-tubulin | 1.1156065 | 1.1865622 | 1.2266594 | 0.9207491 | 0.8935919 | 0.5640225 |
| Stearyl-CoA desaturase, liver | 0.7023494 | 0.8427589 | 2.481802 | 0.8246076 | 0.3749314 | 0.1710327 |
| Glutathione S-transferase theta-1 | 0.8873789 | 0.97986 | 0.8523175 | 1.0059683 | 0.935466 | 1.4088506 |
| alpha-1,2-fucosyltrans-ferase | 0.8367168 | 1.1069651 | 1.277226 | 1.0476928 | 1.06407 | 0.8851402 |
| Phase-1 RCT-138 | 0.8690252 | 0.8993464 | 0.8207451 | 0.9544993 | 0.9424046 | 0.9731333 |
| Phase-1 RCT-295 | 0.8767533 | 1.3531277 | 1.2921563 | 1.0481415 | 1.0397083 | 1.0199283 |
| Phase-1 RCT-164 | 0.9798481 | 0.8152962 | 0.8562899 | 1.1332086 | 0.7796118 | 0.8134604 |
| Alpha-2-macroglobulin, sequence 2 | 1.0800911 | 1.2139634 | 1.1235101 | 1.2047904 | 1.4408623 | 1.4649123 |
| Biliverdin reductase | 0.9535676 | 0.9679392 | 1.0500951 | 0.9378648 | 1.0766612 | 0.8843002 |
| Phase-1 RCT-121 | 1.0905569 | 1.0618148 | 1.0226505 | 1.0971549 | 1.0966954 | 1.1212958 |
| Dynamin-1 (D100) | 0.8880163 | 0.9907992 | 0.8488689 | 1.0051444 | 1.1728771 | 0.9000319 |
| Phase-1 RCT-192 | 1.4003087 | 1.2253195 | 1.0627693 | 0.9629431 | 0.8815428 | 0.9100967 |
| Fatty acid synthase | 0.7087281 | 0.8413119 | 1.6974196 | 0.7474876 | 0.7729672 | 0.4009554 |
| Phase-1 RCT-211 | 0.9566983 | 1.1611409 | 1.0626749 | 0.894598 | 1.0912815 | 1 |
| Ribosomal protein S9 | 0.8471644 | 0.7142426 | 0.8256685 | 0.7124907 | 0.7869399 | 0.7527326 |
| NADP-dependent isocitrate dehydrogenase, | 0.9905917 | 0.9512928 | 0.9443345 | 0.9233404 | 0.6360568 | 0.7327203 |
| cytosolic | | | | | | |
| Phase-1 RCT-57 | 1.0759025 | 1.0915487 | 0.9444861 | 1.0902665 | 1.0498524 | 1.1797404 |
| Phase-1 RCT-36 | 0.9840509 | 0.903018 | 1.1521761 | 0.997174 | 0.8348107 | 0.9335741 |
| Apolipoprotein All | 0.7081034 | 0.9204508 | 0.6026389 | 0.6772602 | 0.9426917 | 1.1240492 |
| Phase-1 RCT-68 | 0.9663038 | 1.1586143 | 1.1170564 | 1.1150395 | 1.4125899 | 1.0801935 |
| Multidrug resistant protein-2 | 1.0947547 | 1.0197006 | 0.9801525 | 0.9621009 | 1.2237284 | 0.9472362 |
| Phase-1 RCT-39 | 1.0464879 | 1.3200861 | 1.1040015 | 1.1565228 | 1.6524477 | 1.1725256 |
| NIPK | 0.9376257 | 0.9464999 | 1.0478529 | 1.1670011 | 1.0838578 | 1.0127963 |
| F1-ATPase beta subunit | 0.8354701 | 0.8527878 | 0.8796774 | 0.785032 | 0.6713193 | 0.7925922 |
| Elongation factor-1 alpha | 0.9002569 | 0.8717752 | 0.841257 | 0.9165599 | 0.6963314 | 0.8901634 |
| Phase-1 RCT-24 | 1.1332122 | 1.7547494 | 1.5823368 | 1.1496176 | 1.0248694 | 0.6563191 |
| Beta-tubulin, class I | 1.0488628 | 1.8479863 | 1.8090361 | 1.1589731 | 1.0304754 | 0.5848316 |
| 14-3-3 zeta | 1.155319 | 1.0730238 | 1.0690773 | 0.9016347 | 0.9141322 | 0.7630782 |
| Alpha-prothymosin | 0.8679537 | 1.1007658 | 0.984184 | 1.068534 | 0.808533 | 0.934819 |
| Phase-1 RCT-58 | 1.7628741 | 2.4260914 | 1.4134598 | 1.3878303 | 1.0686206 | 1.2387108 |
| Phase-1 RCT-290 | 1.2283792 | 1.1904912 | 1.2837416 | 0.7803015 | 0.9012203 | 1.0468981 |
| Transitional endoplasmic reticulum ATPase | 1.1523108 | 0.9411885 | 1.0021294 | 0.9342828 | 0.9776657 | 0.7792234 |
| Beta-actin | 0.8485102 | 1.4414297 | 1.5952727 | 0.92439 | 0.788799 | 0.8780814 |
| Thioredoxin-1 (Trx1) | 0.7376477 | 0.9789224 | 0.957205 | 0.9394296 | 0.810562 | 0.6703984 |
| Phase-1 RCT-174 | 0.9286366 | 0.9869553 | 1.0739694 | 0.9467322 | 0.7626795 | 0.7495541 |
| Beta-actin, sequence 2 | 0.8772548 | 1.2594624 | 1.2245363 | 0.9565426 | 0.7405182 | 0.6703618 |
| Phase-1 RCT-109 | 1.151125 | 1.2275605 | 1.2834669 | 1.0544317 | 1.0959524 | 1.0810595 |
| Phase-1 RCT-154 | 1.022631 | 1.123081 | 1.0528035 | 1.0407375 | 1.1455297 | 0.9646869 |
| Hypoxanthine-guanine | 1.0457658 | 0.974292 | 1.1722118 | 0.8648775 | 0.6428782 | 0.6377174 |
| phosphoribosyltransferase | | | | | | |
| Phase-1 RCT-213 | 1.072504 | 1.140303 | 1.1149786 | 1.0525291 | 1.1365695 | 1.037818 |
| Phase-1 RCT-260 | 1.0430666 | 1.5193281 | 1.1560673 | 1.2946502 | 2.069257 | 1.3677446 |
| Phase-1 RCT-12 | 1.0474133 | 1.2560636 | 1.2557675 | 1.1649806 | 1.0926504 | 0.9731258 |
| Phase-1 RCT-293 | 1.0210024 | 0.926058 | 0.9358627 | 0.9445224 | 0.912496 | 0.8358585 |
| Ribosomal protein S8 | 0.8142046 | 1.2142032 | 1.0584548 | 0.9407015 | 0.857261 | 0.859827 |
| Pyruvate kinase, muscle | 1.0397152 | 1.0603061 | 1.2658142 | 1.0011749 | 1.1648186 | 1.0595326 |
| Nucleoside diphosphate kinase beta isoform | 1.001797 | 1.3771052 | 1.2346809 | 0.9509649 | 0.8371346 | 0.7529434 |
| p55CDC | 1.0557381 | 0.7146177 | 0.8749532 | 1.1316959 | 1.1602806 | 1.0138327 |
| Phase-1 RCT-156 | 1.0020304 | 0.9822069 | 1.0062172 | 1.0507131 | 0.8276653 | 0.9334294 |
| Ribosomal protein L13A | 0.961445 | 1.054638 | 1.1947674 | 1.0526072 | 0.9633626 | 1.0811931 |
| Phase-1 RCT-258 | 1.0627589 | 1.0845777 | 1.0125678 | 1.0348291 | 0.9904645 | 0.9506449 |
| Insulin-like growth factor I | 0.4911268 | 0.8592545 | 0.8795207 | 1.1017507 | 0.5483277 | 0.6091996 |
| Cytochrome P450 3A1 | 0.4495637 | 1.0798403 | 0.4109826 | 0.9488273 | 0.6708425 | 0.8881634 |
| {Ribosomal protein 16} | 0.8429774 | 1.1094832 | 1.1262203 | 0.9057198 | 0.9057905 | 0.9862998 |
| Organic cation transporter 3 | 0.963211 | 1.0725541 | 1.0853885 | 0.8876924 | 0.9249399 | 0.8978446 |
| Calpain 2 | 0.9788865 | 1.1063011 | 1.0847701 | 1.0715489 | 1.2646389 | 1.0660602 |
| Phase-1 RCT-102 | 0.6135958 | 1.0651057 | 0.7739497 | 0.8495645 | 0.7996237 | 0.4361207 |
| Equilbrative nitrobenzylthioinosine-sensitive | 0.733324 | 1.1811428 | 0.9247397 | 1.0435213 | 1.1939234 | 0.9007819 |
| nucleoside transporter | | | | | | |
| Phase-1 RCT-227 | 0.5982322 | 0.9537432 | 0.8302525 | 1.0325209 | 1.0857631 | 0.8765516 |
| N-hydroxy-2-acetylaminofluorene sulfotransferase (ST1C1) | 0.6020333 | 1.1406024 | 0.8783152 | 1.0334685 | 0.7906436 | 0.7683087 |
| Cathepsin S | 0.9779825 | 0.8176972 | 0.7137683 | 0.91483 | 0.6779549 | 0.8153431 |
| Apolipoprotein CIII | 0.729895 | 0.9255832 | 0.9327635 | 0.9102806 | 0.9396611 | 0.8980252 |
| Cytochrome P450 2A3 | 1.1159716 | 0.7373989 | 0.7745938 | 0.8311551 | 1.1817834 | 0.8833851 |
| Cytochrome P450 2C23 | 1.0233607 | 0.7666213 | 0.6876439 | 0.9337705 | 0.6587017 | 1.0180564 |
| Apolipoprotein C1 | 0.4205687 | 0.6511681 | 0.6461373 | 0.8008807 | 0.6854454 | 0.7356579 |
| Betaine homocysteine methyltransferase (BHMT) | 1.3524756 | 1.1330392 | 1.2980897 | 0.6869918 | 0.7574972 | 1.0618377 |
| Paraoxonase 1 | 0.7576053 | 0.8293496 | 0.7777128 | 1.0247835 | 0.7670471 | 0.8772206 |
| Phase-1 RCT-207 | 1.2949879 | 1.2802753 | 1.2479931 | 1.1362997 | 1.3571469 | 1.1681944 |
| Cathepsin B | 1.1710337 | 0.6429157 | 0.7370607 | 0.8340884 | 0.7638789 | 0.8206951 |
| Phase-1 RCT-144 | 0.9812635 | 0.9896805 | 1.0395595 | 0.9292108 | 1.0298191 | 0.9192516 |
| Alpha-1 microglobulin/bikunin precursor (Ambp) | 0.8082636 | 0.7800831 | 0.8236181 | 0.9184158 | 0.6700303 | 0.7475736 |
| Cyclin D1 | 0.8806712 | 0.8905195 | 0.9920145 | 1.2671701 | 0.764463 | 0.6915481 |
| Presenilin-1 | 0.734044 | 0.491666 | 0.6838772 | 0.9354093 | 0.6609998 | 0.6652281 |
| Protein tyrosine phosphatase, receptor type, D | 0.6945829 | 0.6554171 | 0.7499036 | 0.9465961 | 0.6193129 | 0.6625299 |
| Multidrug resistant protein-1 | 1.1132727 | 0.9017593 | 0.9550255 | 0.9597141 | 1.148509 | 0.8973927 |
| Cathepsin L, sequence 2 | 0.9470462 | 1.1565012 | 0.8966908 | 1.0414155 | 1.0620497 | 1.2271235 |
| JNK1 stress activated protein kinase | 0.7708649 | 0.8462283 | 0.8646606 | 0.9543814 | 0.8783579 | 1.0981374 |
| Phase-1 RCT-43 | 1.3047005 | 1.0215228 | 1.0186087 | 1.0316347 | 0.8641629 | 0.9088063 |
| Tissue plasminogen activator | 1.0164875 | 1.02526 | 1.0561055 | 1.016692 | 1.2096735 | 0.9118817 |
| Ubiquitin conjugating enzyme (RAD 6 | 1.6448854 | 1.0887611 | 1.0380757 | 0.8869945 | 0.7987467 | 0.6901038 |
| homologue) | | | | | | |
| Phase-1 RCT-173 | 1.188338 | 1.2198315 | 1.0886016 | 0.9443107 | 1.1436399 | 1.0639615 |
| Ornithine decarboxylase | 0.9452642 | 1.4660763 | 1.5359765 | 1.3232245 | 1.4517137 | 1.2580624 |
| Zinc finger protein | 2.2509618 | 1.0485976 | 0.9030982 | 1.072402 | 1.1401569 | 1.1422974 |
| CDK108 | 0.7569535 | 1.1431019 | 1.0555173 | 1.1088835 | 1.069331 | 0.780015 |
| Phase-1 RCT-117 | 0.8797169 | 0.8397641 | 0.9884815 | 1.0041924 | 0.6569024 | 1.069035 |
| {Phase-1 RCT 98} | 1.016783 | 0.8950634 | 0.9900761 | 1.0371346 | 1.244872 | 0.9431975 |
| Aquaporin-3 (AQP3) | 0.9973809 | 1.0517709 | 1.0150708 | 1.1303658 | 1.584589 | 1.1273606 |
| Cholesterol esterase | 0.5562676 | 0.8492237 | 0.7214252 | 0.9517184 | 0.6175317 | 0.4831047 |
| Ribosomal protein S17 | 0.7669351 | 1.2543111 | 1.1530198 | 0.964956 | 0.8298696 | 0.8609412 |
| Poly(ADP-ribose) polymerase | 1.2794269 | 0.9585888 | 1.0347806 | 0.9171672 | 0.9888369 | 0.9205747 |
| Annexin V | 1.0080514 | 0.9158522 | 0.9419338 | 1.0747107 | 1.1047 | 0.9239167 |
| Phase-1 RCT-33 | 0.8617401 | 0.783736 | 1.1249604 | 0.9455988 | 0.5571223 | 0.8282624 |
| Phase-1 RCT-61 | 1.2016295 | 1.6551956 | 1.2779154 | 1.2894487 | 1.3189981 | 1.2266397 |
| Retinol dehydrogenase type III | 0.8144317 | 0.7694085 | 0.8844379 | 0.9006121 | 0.6697387 | 0.736603 |
| ATPase inhibitor (rat mitochondrial IF1 protein) | 0.6336075 | 0.745707 | 0.8026933 | 0.8055155 | 0.5721975 | 0.5959546 |
| Thymidylate synthase | 0.9674373 | 0.979291 | 1.0095354 | 1.0308467 | 1.2690271 | 1.2930764 |
| Interleukin-18 | 1.090983 | 1.1152735 | 1.1549954 | 1.1817442 | 1.6709224 | 1.2356261 |
| Lecithin: cholesterol acyltransferase | 0.8450127 | 0.8306551 | 0.870392 | 0.9421437 | 0.6713304 | 0.8059924 |
| Contrapsin-like protease inhibitor (CPi-21) | 0.7321507 | 0.9233533 | 0.9633406 | 0.9481024 | 0.726007 | 0.697846 |
| Proliferating cell nuclear antigen gene | 0.8965269 | 0.6881105 | 0.8794885 | 0.9269369 | 0.9263132 | 0.9446375 |
| Phase-1 RCT-230 | 1.0406 | 1.6997038 | 1.409522 | 1.3183954 | 2.3302011 | 1.5084877 |
| Cytochrome P450 2D18 | 0.8402475 | 1.180543 | 0.9105666 | 1.1410987 | 0.9553257 | 1.2370874 |
| Phase-1 RCT-48 | 0.8973675 | 0.9518727 | 0.8384043 | 1.0807776 | 0.7940692 | 0.8099068 |
| Phase-1 RCT-292 | 0.9871337 | 0.9107593 | 0.857292 | 1.0711156 | 1.1163489 | 1.015689 |
| Arginosuccinate synthetase 1 | 1.0175127 | 0.6932984 | 1.0498676 | 0.797556 | 1.0624845 | 1.5216209 |
| C-reactive protein | 0.6838257 | 0.6811762 | 0.7092243 | 0.9274419 | 0.550492 | 0.6492354 |

| | APAP | AZA | AZA | AZA | AZA | AZA | AZA |
|---|---|---|---|---|---|---|---|
| Compound-Dose | 1000 | 50 | 50 | 50 | 200 | 200 | 200 |
| Animal Number | 2139 | 1827 | 1828 | 1829 | 1837 | 1838 | 1839 |
| Liver Toxicity Necrosis Classification | yes | no | no | no | no | no | no |
| Gene Name | | | | | | | |
| Osteoactivin | 1.7442634 | 1.0554289 | 1.2178595 | 1.1061507 | 0.9829723 | 1.0677098 | 1.2427696 |
| Stathmin | 1.0159887 | 0.9306584 | 1.0516781 | 0.9389561 | 0.9511749 | 0.8807868 | 1.0246967 |
| Calpactin I heavy chain | 1.2721559 | 1.316806 | 1.1392441 | 1.3593363 | 1.2592739 | 1.063146 | 1.0914886 |
| Phase-1 RCT-179 | 0.8476872 | 0.8834053 | 0.9725468 | 1.1693795 | 0.8216287 | 0.7650199 | 0.8047909 |
| Phase-1 RCT-107 | 1.3291122 | 1.209793 | 0.7831907 | 0.7314231 | 0.7404692 | 1.0209668 | 0.975454 |
| Heme oxygenase | 1.3889909 | 0.8902127 | 1.4705927 | 1.4730558 | 0.9848958 | 0.8494319 | 0.9367114 |
| Collagen type II | 0.8960091 | 1.2375706 | 1.0901641 | 0.7014595 | 0.4536103 | 0.7017102 | 0.8396029 |
| Phase-1 RCT-81 | 0.8124965 | 1.4526607 | 1.0298101 | 0.972189 | 0.909838 | 1.060174 | 1.0405328 |
| Glycine methyltransferase | 1.5085021 | 1.3030987 | 0.6120088 | 0.399905 | 0.5086768 | 1.1247524 | 0.7871756 |
| Cystatin C | 0.9823004 | 0.7914807 | 1.017546 | 0.8303401 | 0.7222297 | 0.8299626 | 0.5768945 |
| Melanoma-associated antigen ME491 | 1.1354219 | 0.9531214 | 1.0520972 | 0.8394347 | 1.0992347 | 1.5119162 | 0.8816481 |
| Phase-1 RCT-146 | 1.774253 | 0.9065082 | 0.9689874 | 0.9016306 | 1.0819753 | 0.8883901 | 1.0597003 |
| Gamma-actin, cytoplasmic | 1.040697 | 1.0298547 | 1.2704685 | 0.8135639 | 0.7754642 | 0.7522932 | 0.8322755 |
| Adenine nucleotide translocator 1 | 0.7857239 | 1.1415877 | 0.8038001 | 0.9104329 | 1.15262 | 0.6767545 | 0.5804666 |
| Thymosin beta-10 | 0.9482792 | 0.61918 | 0.8992539 | 1.2631198 | 0.9798922 | 0.6691499 | 0.6478322 |
| Insulin-like growth factor I, exon 6 | 0.690232 | 0.7707499 | 1.3099772 | 0.4849487 | 0.5457941 | 1.0188065 | 1.1790028 |
| Phase-1 RCT-78 | 0.8340079 | 1.2859026 | 1.059758 | 0.7580867 | 0.912423 | 1.1206205 | 1.1363213 |
| Alpha-2-microglobulin | 0.3643026 | 0.9130117 | 0.3729512 | 0.0618712 | 0.1258987 | 0.6250716 | 0.4395205 |
| High affinity IgE receptor gamma chain | 1.0743184 | 0.8991705 | 1.7539 | 1.024774 | 0.9321016 | 1.2125877 | 1.0862519 |
| (FcERIgamma) | | | | | | | |
| 60S ribosomal protein L6 | 0.856624 | 0.7753903 | 1.0650977 | 1.821971 | 1.5385334 | 0.6568363 | 0.6301947 |
| IgE binding protein | 1.4182585 | 0.9969645 | 1.1466537 | 1.2180369 | 1.2516683 | 1.0256358 | 1.1155921 |
| Phase-1 RCT-149 | 1.190779 | 1.5265349 | 1.3824137 | 1.5153292 | 1.3272064 | 1.3338541 | 1.3954105 |
| Cytochrome P450 2C11 | 0.1045564 | 0.4590415 | 0.5969157 | 0.0952107 | 0.1804035 | 0.8313705 | 0.6447482 |
| Uncoupling protein 2 | 0.6527032 | 0.6979151 | 1.203821 | 1.2096642 | 0.9856048 | 0.8054089 | 0.8980107 |
| Cofilin | 0.8447343 | 1.0200111 | 1.4226388 | 1.1968278 | 1.2944959 | 1.2010138 | 1.2559023 |
| Alpha-tubulin | 0.7139607 | 0.8397889 | 1.1861697 | 1.5468941 | 1.3872929 | 0.6289664 | 0.7081554 |
| Stearyl-CoA desaturase, liver | 0.0646366 | 1.000766 | 1.2432685 | 0.0546449 | 0.0907877 | 0.9243081 | 1.0666881 |
| Glutathione S-transferase theta-1 | 1.0786526 | 1.1214896 | 0.8422167 | 1.1615282 | 0.8272302 | 0.8051523 | 0.6895698 |
| alpha-1,2- fucosyltransferase | 0.9286404 | 0.3937459 | 0.7459428 | 0.4797273 | 0.490614 | 0.7848902 | 0.8735161 |
| Phase-1 RCT-138 | 1.188949 | 1.1510736 | 1.3839371 | 1.0106406 | 1.181156 | 1.4436628 | 1.1851641 |
| Phase-1 RCT-295 | 1.0669395 | 1.1803529 | 1.3020525 | 1.8957951 | 1.3770385 | 1.2251912 | 1.1324469 |
| Phase-1 RCT-164 | 0.7117347 | 0.7455668 | 0.7528336 | 0.567269 | 0.7206088 | 0.8474892 | 0.8915803 |
| Alpha-2-macroglobulin, sequence 2 | 1.3104239 | 1.3149582 | 1.2127981 | 1.4095447 | 1.1061646 | 1.2582529 | 1.143172 |
| Biliverdin reductase | 1.0306371 | 0.8921737 | 1.3772447 | 1.2323927 | 1.2334658 | 1.0485396 | 1.0589442 |
| Phase-1 RCT-121 | 1.0358386 | 0.7792645 | 0.9612248 | 0.7360094 | 0.7708997 | 0.7584004 | 0.8255143 |
| Dynamin-1 (D100) | 0.8220473 | 0.9758607 | 0.8964437 | 0.616138 | 0.9293029 | 1.1155728 | 1.0274137 |
| Phase-1 RCT-192 | 1.039308 | 1.1140817 | 1.2607106 | 3.4208527 | 2.4464014 | 1.004794 | 1.0424356 |
| Fatty acid synthase | 0.468046 | 1.5044891 | 1.503357 | 0.2159105 | 0.3805859 | 0.9686304 | 1.2078505 |
| Phase-1 RCT-211 | 0.9856777 | 1.178419 | 1.0099635 | 1.2506936 | 1.0778153 | 0.845866 | 0.8458847 |
| Ribosomal protein S9 | 0.6113282 | 0.8741314 | 1.0451373 | 1.3967657 | 1.3615512 | 0.6378786 | 0.5158105 |
| NADP-dependent isocitrate dehydrogenase, | 0.7281815 | 1.1164416 | 1.12042 | 0.9136791 | 0.9594753 | 1.0586321 | 1.1740109 |
| cytosolic | | | | | | | |
| Phase-1 RCT-57 | 1.0201224 | 0.9202775 | 0.7485843 | 0.8463665 | 0.8738675 | 0.7581104 | 0.8574721 |
| Phase-1 RCT-36 | 0.9619991 | 1.1618592 | 1.0629506 | 0.9393255 | 0.9162824 | 1.1687043 | 1.0730476 |
| Apolipoprotein All | 0.6442748 | 0.5168222 | 0.4584646 | 0.3165645 | 0.3875718 | 0.4653184 | 0.358289 |
| Phase-1 RCT-68 | 1.1241931 | 1.1218619 | 1.1562414 | 1.4213893 | 1.2576603 | 1.0669141 | 1.1530737 |
| Multidrug resistant protein-2 | 1.1750672 | 1.0418181 | 1.354428 | 1.8041688 | 1.2948089 | 1.1766388 | 1.116643 |
| Phase-1 RCT-39 | 1.2318007 | 0.9167102 | 0.9317025 | 1.2686477 | 1.0485166 | 0.7530326 | 0.7882171 |
| NIPK | 0.9207399 | 1.0612721 | 1.1627171 | 1.363865 | 0.7772798 | 1.0631816 | 1.1023109 |
| F1-ATPase beta subunit | 0.782541 | 0.7831922 | 1.1518492 | 0.9349127 | 1.0132805 | 1.1826826 | 0.7791532 |
| Elongation factor-1 alpha | 0.8212068 | 0.7677893 | 1.2166913 | 1.2718514 | 0.9078913 | 1.0476997 | 0.9326742 |
| Phase-1 RCT-24 | 0.9129595 | 1.1502246 | 1.325308 | 2.2779536 | 2.1301224 | 1.198025 | 1.4939308 |
| Beta-tubulin, class I | 0.8126831 | 1.3353673 | 1.7633778 | 2.7758663 | 2.386019 | 1.4851009 | 1.848582 |
| 14-3-3 zeta | 0.7902211 | 0.6612386 | 1.0777806 | 1.1592737 | 1.0504484 | 0.8689126 | 0.9500805 |
| Alpha-prothymosin | 0.8622433 | 0.8872735 | 1.1767995 | 0.7982293 | 0.7901604 | 1.0041946 | 1.0139039 |
| Phase-1 RCT-58 | 1.9065506 | 1.5671803 | 1.2977611 | 1.5052344 | 1.657196 | 0.5507801 | 1.0935087 |
| Phase-1 RCT-290 | 1.0416876 | 1.4113704 | 1.1465575 | 0.5699361 | 0.56765 | 1.0782679 | 1.0230234 |
| Transitional endoplasmic reticulum ATPase | 0.8262833 | 0.9065571 | 1.0192837 | 0.948592 | 1.1002098 | 1.0831057 | 0.9518993 |
| Beta-actin | 1.0552626 | 0.7541703 | 1.1531096 | 0.7794975 | 0.7793074 | 0.6061226 | 0.5811976 |
| Thioredoxin-1 (Trx1) | 0.7697759 | 0.8737466 | 0.9286034 | 0.5324479 | 1.0580884 | 0.8255696 | 0.5905672 |
| Phase-1 RCT-174 | 0.7754786 | 0.8046408 | 0.8587431 | 0.861261 | 0.9573403 | 1.0298346 | 1.0853034 |
| Beta-actin, sequence 2 | 0.8591993 | 1.0326557 | 0.9637842 | 0.8829244 | 0.874554 | 0.6541884 | 0.687307 |
| Phase-1 RCT-109 | 1.0445235 | 0.9504346 | 0.9968053 | 2.1473334 | 1.7045947 | 1.0164179 | 0.9694553 |
| Phase-1 RCT-154 | 1.0275319 | 1.0133494 | 1.0358539 | 2.0430875 | 1.5587504 | 0.9866743 | 1.0898685 |
| Hypoxanthine-guanine | 0.6773862 | 0.7274668 | 1.0639428 | 1.0326027 | 1.046509 | 0.8281386 | 0.8289464 |
| phosphoribosyltransferase | | | | | | | |
| Phase-1 RCT-213 | 0.9762197 | 0.9076636 | 0.9433808 | 1.3587458 | 1.5025436 | 0.944785 | 1.0605147 |
| Phase-1 RCT-260 | 1.5011295 | 1.0022395 | 0.799633 | 0.8571042 | 0.9034081 | 0.8561917 | 0.9838793 |
| Phase-1 RCT-12 | 1.0808262 | 1.6503576 | 1.4280672 | 1.695876 | 1.5099487 | 1.3035158 | 1.3855301 |
| Phase-1 RCT-293 | 0.9324133 | 1.1118791 | 1.1774235 | 0.9224728 | 0.947503 | 1.1856179 | 1.159112 |
| Ribosomal protein S8 | 0.9479709 | 1.0793735 | 1.3530215 | 2.7213876 | 2.2420235 | 1.0352346 | 0.8513129 |
| Pyruvate kinase, muscle | 1.0628451 | 1.3330733 | 1.2195853 | 1.4377903 | 0.8537456 | 1.0729389 | 1.0741525 |
| Nucleoside diphosphate kinase beta isoform | 1.0072038 | 1.4325424 | 1.224493 | 3.3242455 | 3.802832 | 1.3467633 | 1.1831572 |
| p55CDC | 0.9107812 | 1.4588141 | 0.9388015 | 1.0718423 | 0.9008254 | 0.8605592 | 1.0412344 |
| Phase-1 RCT-156 | 0.8604745 | 1.6647955 | 0.9396495 | 1.3056443 | 1.2394054 | 1.096613 | 0.9209001 |
| Ribosomal protein L13A | 0.9844711 | 0.6753593 | 1.170484 | 1.4403586 | 1.361612 | 0.9932966 | 0.9373242 |
| Phase-1 RCT-258 | 1 | 0.8810417 | 0.8165872 | 1.1835061 | 1.104901 | 0.8072848 | 0.9084871 |
| Insulin-like growth factor I | 0.646507 | 0.5920734 | 1.0678031 | 0.3703534 | 0.463187 | 0.820389 | 0.9417176 |
| Cytochrome P450 3A1 | 0.4752716 | 0.6744881 | 0.4381978 | 0.1746416 | 0.2511769 | 0.8430821 | 0.2194159 |
| {Ribosomal protein 16} | 1.0031577 | 1.0102376 | 1.188913 | 2.2601793 | 1.5788366 | 1.0031677 | 0.9016348 |
| Organic cation transporter 3 | 0.8036549 | 0.7049683 | 0.8244577 | 1.546774 | 1.1664284 | 0.743149 | 0.8063387 |
| Calpain 2 | 1.1155587 | 1.097653 | 1.097323 | 1.285774 | 1.050767 | 1.1031994 | 1.0970914 |
| Phase-1 RCT-102 | 0.4074023 | 0.5818879 | 0.762001 | 0.1889717 | 0.2210883 | 0.5289453 | 0.6473869 |
| Equilbrative nitrobenzylthioinosine-sensitive | 0.7375059 | 1.2227075 | 0.8464594 | 0.6160725 | 0.7488957 | 0.8663853 | 0.6690838 |
| nucleoside transporter | | | | | | | |
| Phase-1 RCT-227 | 0.6859568 | 1.0967706 | 0.9620442 | 0.359174 | 0.587422 | 1.3050625 | 0.7302176 |
| N-hydroxy-2-acetylaminofluorene | 0.6157163 | 0.9282182 | 0.8349424 | 0.4179207 | 0.5293017 | 0.8259302 | 0.5801202 |
| sulfotransferase (ST1C1) | | | | | | | |
| Cathepsin S | 0.9907197 | 0.7129872 | 1.2409745 | 1.021293 | 0.9798672 | 1.0067582 | 0.9086005 |
| Apolipoprotein CIII | 0.6684836 | 0.8955339 | 0.6349268 | 0.3930081 | 0.5159372 | 0.7510509 | 0.5855129 |
| Cytochrome P450 2A3 | 0.5621319 | 1.6945022 | 0.7974555 | 0.4235895 | 0.3482875 | 1.1394193 | 0.6888857 |
| Cytochrome P450 2C23 | 0.8889001 | 0.6807263 | 0.7289705 | 0.3716227 | 0.4906106 | 0.7576244 | 0.6330579 |
| Apolipoprotein C1 | 0.7231054 | 1.7403597 | 0.8598684 | 0.5578179 | 0.8647355 | 1.3603568 | 0.9164415 |
| Betaine homocysteine methyltransferase (BHMT) | 1.0233914 | 1.6104037 | 1.1487464 | 0.2509724 | 0.2264788 | 1.1887648 | 0.8340493 |
| Paraoxonase 1 | 0.9011304 | 0.6819964 | 1.0109458 | 0.30283 | 0.4097163 | 0.9884542 | 0.6583744 |
| Phase-1 RCT-207 | 1.0074805 | 0.7409238 | 0.7238606 | 1.291643 | 1.1477638 | 0.6706258 | 0.7480131 |
| Cathepsin B | 0.9178546 | 0.8575382 | 1.0394129 | 0.8696116 | 0.8366765 | 1.1670539 | 0.8365765 |
| Phase-1 RCT-144 | 0.8789157 | 0.8155246 | 0.9704807 | 1.0970395 | 1.0610635 | 1.0761682 | 1.0535872 |
| Alpha-1 microglobulin/bikunin precursor (Ambp) | 0.7101873 | 1.5115155 | 1.0155206 | 0.8410222 | 0.9051377 | 1.1796942 | 0.9260746 |
| Cyclin D1 | 0.7693539 | 1.1415607 | 0.7696232 | 1.1807173 | 1.2562618 | 1.805082 | 0.7428854 |
| Presenilin-1 | 0.6836659 | 1.9513704 | 0.7028016 | 0.323649 | 0.3902094 | 1.0423739 | 0.9264341 |
| Protein tyrosine phosphatase, receptor type, D | 0.8857953 | 1.0473572 | 1.0427297 | 0.6371996 | 0.7863677 | 0.9425601 | 0.8545969 |
| Multidrug resistant protein-1 | 1.0323625 | 1.3183824 | 1.2401835 | 1.7496647 | 1.1415226 | 1.1742604 | 0.9810166 |
| Cathepsin L, sequence 2 | 1.167783 | 0.7857251 | 1.1075039 | 2.5656836 | 1.2579716 | 0.9314608 | 0.790931 |
| JNK1 stress activated protein kinase | 1.1059991 | 0.5010416 | 0.8786856 | 0.4886538 | 0.6700397 | 0.8652743 | 0.7284654 |
| Phase-1 RCT-43 | 0.8379463 | 1.1425397 | 0.8792898 | 0.9462395 | 0.9932675 | 0.7075815 | 0.7606553 |
| Tissue plasminogen activator | 0.9832287 | 0.957346 | 0.9367948 | 0.967958 | 1.0479386 | 1.0272671 | 1.069055 |
| Ubiquitin conjugating enzyme (RAD 6 | 0.702547 | 0.7409223 | 0.8055913 | 1.4088408 | 1.4368312 | 0.7029698 | 0.733473 |
| homologue) | | | | | | | |
| Phase-1 RCT-173 | 1.0490872 | 0.7328906 | 0.5982693 | 0.7436121 | 1.2938107 | 0.7632562 | 0.7520759 |
| Ornithine decarboxylase | 1.2993973 | 1.1187645 | 1.1806861 | 2.616302 | 1.6335962 | 0.9989513 | 1.1287221 |
| Zinc finger protein | 1.0420237 | 0.8737479 | 0.8886522 | 1.1009141 | 0.8656356 | 0.7057217 | 0.7678338 |
| CDK108 | 0.8663761 | 0.7623443 | 0.6379975 | 0.8252998 | 0.7740063 | 0.6578887 | 0.7161561 |
| Phase-1 RCT-117 | 1.0627179 | 0.8720619 | 0.6293701 | 0.7202129 | 0.8540639 | 1.0605592 | 0.8119791 |
| {Phase-1 RCT 98} | 0.8672158 | 1.2261211 | 1.0002141 | 0.8830265 | 0.946893 | 1.0899508 | 1.0668103 |
| Aquaporin-3 (AQP3) | 1.1014991 | 1.2479612 | 0.9537413 | 0.9330002 | 0.916423 | 1.1236649 | 1.0545712 |
| Cholesterol esterase | 0.4625594 | 0.706654 | 0.4870578 | 0.2103164 | 0.3085011 | 0.5588734 | 0.2597467 |
| Ribosomal protein S17 | 0.9603876 | 0.7621805 | 1.268101 | 2.3542352 | 1.9554137 | 1.0609764 | 0.9118373 |
| Poly(ADP-ribose) polymerase | 0.8974724 | 0.9959393 | 1.1090475 | 1.2187282 | 1.2706858 | 0.994122 | 1.0457635 |
| Annexin V | 0.9556932 | 0.9545428 | 0.9269959 | 0.9488539 | 0.7602707 | 0.8558553 | 0.9926097 |
| Phase-1 RCT-33 | 0.8638663 | 0.901396 | 1.2080189 | 0.6763406 | 0.7120999 | 1.2451891 | 1.078493 |
| Phase-1 RCT-61 | 1.5628604 | 0.9632025 | 0.9213529 | 1.1017598 | 1.0314273 | 0.8044772 | 0.9443607 |
| Retinol dehydrogenase type III | 0.6896768 | 1.0250745 | 0.7365889 | 0.7743334 | 0.9967389 | 1.16051 | 1.0204163 |
| ATPase inhibitor (rat mitochondrial IF1 protein) | 0.6327341 | 0.9077777 | 0.7224093 | 0.2319285 | 0.4608362 | 0.8664358 | 0.8431859 |
| Thymidylate synthase | 1.2923088 | 0.9309375 | 0.8648577 | 1.0367032 | 1.0388154 | 0.921007 | 0.9948289 |
| Interleukin-18 | 1.1971518 | 0.9204744 | 0.7773304 | 0.8539949 | 0.9944 | 0.8527051 | 0.8519948 |
| Lecithin: cholesterol acyltransferase | 0.7822452 | 0.8543268 | 1.0965284 | 0.6879731 | 0.9173967 | 1.1360074 | 1.1017723 |
| Contrapsin-like protease inhibitor (CPi-21) | 0.371978 | 1.2212936 | 0.9761204 | 0.28659 | 0.5100985 | 1.0058509 | 0.665351 |
| Proliferating cell nuclear antigen gene | 0.8397319 | 0.7572033 | 0.9464664 | 0.9477596 | 0.9220185 | 0.8818064 | 0.9607692 |
| Phase-1 RCT-230 | 1.499964 | 0.9254345 | 0.9686552 | 0.9083772 | 0.8529886 | 0.8197033 | 1.003342 |
| Cytochrome P450 2D18 | 1.04806 | 1.1142033 | 0.9355603 | 0.9916465 | 1.1771287 | 1.2717947 | 0.9140303 |
| Phase-1 RCT-48 | 0.9119663 | 1.0065289 | 1.1119583 | 0.6569394 | 0.6744658 | 0.8815128 | 0.7978674 |
| Phase-1 RCT-292 | 1.0922023 | 1.1881014 | 1.3417306 | 1.0838637 | 1.1884102 | 1.1078424 | 1.0187814 |
| Arginosuccinate synthetase 1 | 1.1606932 | 1.4951184 | 1.0026661 | 0.9561262 | 0.7604604 | 1.3828661 | 1.1822226 |
| C-reactive protein | 0.9538879 | 0.7238764 | 1.1308758 | 0.5118333 | 0.7231404 | 0.8444573 | 0.8774066 |

| | BAP | BAP | BAP | BEN | BEN | BEN |
|---|---|---|---|---|---|---|
| Compound-Dose | 30 | 30 | 30 | 250 | 250 | 250 |
| Animal Number | 2347 | 2348 | 2349 | 2027 | 2028 | 2029 |
| Liver Toxicity Necrosis Classification | no | no | no | no | no | no |
| Gene Name | | | | | | |
| Osteoactivin | 0.7633666 | 0.9208267 | 0.9368166 | 1.0276345 | 0.9759598 | 0.9541799 |
| Stathmin | 1.0648342 | 0.9941376 | 0.9678392 | 1.0049565 | 0.9890381 | 0.9954862 |
| Calpactin I heavy chain | 0.8174328 | 1.0142207 | 1.1011418 | 0.8451812 | 0.8605818 | 0.8675928 |
| Phase-1 RCT-179 | 0.9040349 | 1.1635467 | 0.8116379 | 1.1476456 | 1.1285342 | 1.1974391 |
| Phase-1 RCT-107 | 0.7142405 | 0.810172 | 0.7349405 | 1.1676675 | 0.7332417 | 0.6700099 |
| Heme oxygenase | 0.8515387 | 1.1506398 | 0.9976617 | 0.8731903 | 0.8692938 | 0.8814703 |
| Collagen type II | 0.615554 | 0.714283 | 0.8448618 | 0.7732662 | 0.9583902 | 0.9502413 |
| Phase-1 RCT-81 | 1.0302714 | 1.0626395 | 1.018509 | 1.0503477 | 1.0577362 | 1.0471642 |
| Glycine methyltransferase | 0.7321743 | 0.8491656 | 0.7744513 | 1.3632704 | 0.6994995 | 0.5862431 |
| Cystatin C | 1.0605918 | 1.4650395 | 1.222467 | 1.0179199 | 0.9486784 | 1.0671415 |
| Melanoma-associated antigen ME491 | 0.4696938 | 0.7457984 | 1.0950565 | 0.9483482 | 1.0365373 | 0.8786986 |
| Phase-1 RCT-146 | 0.8189076 | 0.820985 | 0.7638132 | 0.824985 | 0.9454404 | 0.9142026 |
| Gamma-actin, cytoplasmic | 1.199802 | 1.0019974 | 0.9828488 | 1.1360788 | 0.9045605 | 1.1049803 |
| Adenine nucleotide translocator 1 | 0.7226421 | 0.8928698 | 0.834797 | 1.1944734 | 1.0293244 | 1.2970784 |
| Thymosin beta-10 | 1.119317 | 1.1637752 | 1.2710003 | 0.8949719 | 1.0009009 | 0.9454685 |
| Insulin-like growth factor I, exon 6 | 1.0761082 | 0.9493622 | 1.001006 | 0.8363254 | 1.2708176 | 1.3499405 |
| Phase-1 RCT-78 | 0.9468452 | 0.8728916 | 0.9863577 | 1.1865907 | 0.9708062 | 1.0264778 |
| Alpha-2-microglobulin | 0.7410244 | 1.4263014 | 0.7040035 | 2.2245996 | 0.8669123 | 1.103959 |
| High affinity IgE receptor gamma chain | 0.836903 | 1.17582 | 0.931718 | 1.0306318 | 1.0805396 | 1.0324378 |
| (FcERIgamma) | | | | | | |
| 60S ribosomal protein L6 | 1.0829839 | 1.2432252 | 1.1669441 | 0.951027 | 0.9719621 | 0.9743241 |
| IgE binding protein | 0.9529634 | 1.2088739 | 1.1500067 | 0.975083 | 1.0199732 | 1.0596234 |
| Phase-1 RCT-149 | 0.8934012 | 0.8470944 | 0.8776645 | 0.9005011 | 1.0129972 | 0.9334681 |
| Cytochrome P450 2C11 | 0.7171882 | 0.9117176 | 0.7835012 | 1.3090224 | 0.7224247 | 1.1185842 |
| Uncoupling protein 2 | 0.9362439 | 1.1254663 | 1.1691501 | 0.8283706 | 0.9016534 | 0.9460508 |
| Cofilin | 1.0651364 | 1.0518005 | 1.1789145 | 1.1164256 | 1.135401 | 1.2132947 |
| Alpha-tubulin | 1.1650293 | 1.1102129 | 1.145896 | 0.9460027 | 0.7935599 | 0.8174322 |
| Stearyl-CoA desaturase, liver | 0.5249705 | 0.2157343 | 0.8806791 | 1.3591728 | 1.0573429 | 1.2004994 |
| Glutathione S-transferase theta-1 | 1.637086 | 0.9991132 | 1.1300261 | 1.1124417 | 1.0428691 | 0.9949669 |
| alpha-1,2-fucosyltransferase | 1.6037846 | 1.3237587 | 1.5309525 | 0.9678453 | 1.2194093 | 1.130337 |
| Phase-1 RCT-138 | 0.8086864 | 1.0814918 | 1.0210139 | 0.9874908 | 1.047683 | 0.9956749 |
| Phase-1 RCT-295 | 1.2575265 | 1.2619334 | 1.3226562 | 1.1243664 | 1.1032137 | 1.1661669 |
| Phase-1 RCT-164 | 1.0568389 | 1.1784492 | 1.3834275 | 1.0867054 | 1.1984155 | 1.0400144 |
| Alpha-2- macroglobulin, sequence 2 | 0.9095306 | 0.7607313 | 0.8935307 | 1.2206517 | 1.2224475 | 1.1338114 |
| Biliverdin reductase | 1.0420711 | 1.1145922 | 1.1027629 | 0.8827623 | 0.8888741 | 0.797082 |
| Phase-1 RCT-121 | 0.6282476 | 0.9300463 | 0.6098528 | 0.9160819 | 0.8821367 | 0.8802567 |
| Dynamin-1 (D100) | 0.8358082 | 0.8813726 | 0.8808237 | 1.2150725 | 1.0570892 | 1.0090847 |
| Phase-1 RCT-192 | 0.7508628 | 1.0164351 | 0.7197703 | 1.0746738 | 1.0390316 | 1.0715344 |
| Fatty acid synthase | 0.2995672 | 0.2580992 | 0.5391581 | 1.749277 | 0.9031355 | 0.8055428 |
| Phase-1 RCT-211 | 0.9743958 | 1.0029863 | 0.8959709 | 0.9242735 | 0.908486 | 0.9946749 |
| Ribosomal protein S9 | 1.3490418 | 1.1576551 | 1.4237312 | 0.8929449 | 1.1997304 | 0.9336507 |
| NADP-dependent isocitrate dehydrogenase, | 1.3765702 | 1.1915616 | 1.0954084 | 1.0534599 | 1.2545471 | 1.3635904 |
| cytosolic | | | | | | |
| Phase-1 RCT-57 | 0.8414945 | 0.9532251 | 0.9110932 | 1.0412693 | 0.8594852 | 0.8029 |
| Phase-1 RCT-36 | 0.9638533 | 0.7679087 | 0.8034489 | 0.9724424 | 0.9534135 | 1.0073488 |
| Apolipoprotein All | 0.5767774 | 0.6007274 | 0.4518142 | 1.489308 | 0.9021405 | 1.142229 |
| Phase-1 RCT-68 | 1.1840075 | 1.1381367 | 1.0322258 | 0.9488069 | 0.9996616 | 0.9113449 |
| Multidrug resistant protein-2 | 1.5343723 | 1.5238523 | 1.1975721 | 0.808524 | 0.8715478 | 0.9262476 |
| Phase-1 RCT-39 | 0.693597 | 0.9221368 | 0.7676128 | 0.9056468 | 0.900655 | 0.9215154 |
| NIPK | 0.9437876 | 0.9448481 | 0.8842108 | 0.8253925 | 1.1172214 | 1.1365062 |
| F1-ATPase beta subunit | 1.542917 | 1.3481326 | 1.5664926 | 1.2559751 | 1.1916162 | 1.2604909 |
| Elongation factor-1 alpha | 1.4465233 | 1.4719706 | 1.7525318 | 1.2298503 | 1.1063725 | 1.1785532 |
| Phase-1 RCT-24 | 0.6903296 | 0.7265795 | 0.6752951 | 0.9452215 | 0.8406493 | 0.7723461 |
| Beta-tubulin, class I | 1.4545242 | 1.319466 | 1.392385 | 0.9083484 | 0.9226453 | 0.788748 |
| 14-3-3 zeta | 0.9496937 | 0.8965724 | 0.9346451 | 1.0003654 | 0.841206 | 0.8152799 |
| Alpha- prothymosin | 0.9846683 | 0.9794615 | 1.2747164 | 1.2571452 | 1.1483152 | 1.1435899 |
| Phase-1 RCT-58 | 1.2615849 | 1.3819846 | 1.4600791 | 1.0142372 | 1.5507658 | 1.2340151 |
| Phase-1 RCT-290 | 0.9825976 | 0.9644496 | 1.1854625 | 1.2843778 | 0.9691268 | 0.9972787 |
| Transitional endoplasmic reticulum ATPase | 0.8820166 | 1.0974638 | 1.0368942 | 0.9798068 | 0.9246513 | 0.9250945 |
| Beta-actin | 0.806136 | 0.8541617 | 1.2246727 | 1.0360985 | 0.5052073 | 0.7406613 |
| Thioredoxin-1 (Trx1) | 1.3043259 | 1.4216907 | 1.3825355 | 1.041829 | 1.0524304 | 1.1187571 |
| Phase-1 RCT-174 | 1.0753032 | 0.9681437 | 1.2106129 | 1.1553631 | 1.002733 | 1.0731357 |
| Beta-actin, sequence 2 | 0.9866294 | 1.1742547 | 1.0608207 | 1.1561868 | 0.9565587 | 1.0054355 |
| Phase-1 RCT-109 | 1.0879145 | 1.0459028 | 1.0469099 | 0.9323275 | 1.0329255 | 1.0110549 |
| Phase-1 RCT-154 | 0.7850464 | 1.0187016 | 0.8785542 | 0.9946966 | 0.965987 | 0.9396216 |
| Hypoxanthine-guanine | 1.0203968 | 0.9079697 | 1.1034418 | 0.8857088 | 0.826262 | 0.7554023 |
| phosphoribosyltransferase | | | | | | |
| Phase-1 RCT-213 | 0.9384428 | 0.9508672 | 0.9556114 | 1.0514547 | 0.8845639 | 0.9005427 |
| Phase-1 RCT-260 | 0.6325483 | 0.7240926 | 0.6863986 | 0.840439 | 0.8914221 | 0.8637989 |
| Phase-1 RCT-12 | 0.900623 | 0.8641406 | 0.8976966 | 0.9235718 | 0.8781523 | 0.8719189 |
| Phase-1 RCT-293 | 1.2008556 | 1.3799914 | 1.197904 | 0.8535771 | 1.0210873 | 0.9496139 |
| Ribosomal protein S8 | 1.384335 | 1.3501385 | 1.369075 | 1.1272455 | 1.2637554 | 1.2709147 |
| Pyruvate kinase, muscle | 0.8257783 | 1.022199 | 0.9039571 | 0.8465579 | 0.9680424 | 0.8573387 |
| Nucleoside diphosphate kinase beta isoform | 1.2286618 | 1.0921459 | 1.1741147 | 0.9302101 | 1.030918 | 0.9380457 |
| p55CDC | 1.459468 | 1.0146667 | 0.8907179 | 0.6879033 | 0.9709503 | 0.8069004 |
| Phase-1 RCT-156 | 1.0005314 | 0.7993933 | 0.672004 | 1.1551683 | 0.9625714 | 1.0862372 |
| Ribosomal protein L13A | 1.2991463 | 1.173313 | 1.3156627 | 0.9026316 | 1.0166959 | 0.9522156 |
| Phase-1 RCT-258 | 0.8954216 | 1.0146154 | 0.8780399 | 1.065851 | 1.0338187 | 1.0120478 |
| Insulin-like growth factor I | 1.1034846 | 0.9059047 | 0.9288757 | 0.787383 | 1.3650273 | 1.3056902 |
| Cytochrome P450 3A1 | 1.0526843 | 1.4706197 | 1.5553839 | 1.9936191 | 0.7059084 | 1.7198875 |
| {Ribosomal protein 16} | 1.2995632 | 1.2323554 | 1.2353194 | 1.0856886 | 1.0876548 | 1.1374096 |
| Organic cation transporter 3 | 1.0102473 | 1.1715605 | 1.063453 | 0.9688837 | 1.0155559 | 1.0046504 |
| Calpain 2 | 0.9946813 | 0.9305722 | 1.0079275 | 0.9626091 | 1.0058085 | 0.9749764 |
| Phase-1 RCT-102 | 0.6190575 | 0.748213 | 0.5519697 | 0.9401613 | 1.3758427 | 1.0997819 |
| Equilbrative nitrobenzylthioinosine-sensitive | 0.8875864 | 1.0638837 | 0.9308561 | 1.2360958 | 0.9348295 | 1.2593366 |
| nucleoside transporter | | | | | | |
| Phase-1 RCT-227 | 1.1867534 | 1.1043403 | 1.3853403 | 1.6694032 | 1.0077701 | 1.0667775 |
| N-hydroxy-2-acetylaminofluorene | 0.8903882 | 1.2770107 | 0.9767152 | 1.2624698 | 0.9513363 | 1.4696425 |
| sulfotransferase (ST1C1) | | | | | | |
| Cathepsin S | 1.0877686 | 1.6541262 | 1.1219262 | 1.0375466 | 1.1290579 | 1.035276 |
| Apolipoprotein CIII | 0.8705465 | 0.8581371 | 1.0271734 | 1.2714211 | 1.0161208 | 1.0476782 |
| Cytochrome P450 2A3 | 1.7927138 | 1.4152725 | 1.0939548 | 1.0122858 | 1.7618896 | 1.2092849 |
| Cytochrome P450 2C23 | 1.087542 | 1.2884505 | 1.2465564 | 1.0942745 | 1.2156143 | 1.2613577 |
| Apolipoprotein C1 | 0.8559626 | 1.1873101 | 0.939979 | 1.4877951 | 1.2562401 | 0.870997 |
| Betaine homocysteine methyltransferase (BHMT) | 1.2531416 | 1.1022598 | 1.6492082 | 1.3290441 | 0.9210621 | 0.9933548 |
| Paraoxonase 1 | 1.9553347 | 1.7934949 | 1.8180097 | 1.0476077 | 1.2649457 | 1.4494368 |
| Phase-1 RCT-207 | 0.7774006 | 0.9309809 | 0.9347801 | 0.9220811 | 0.808013 | 0.7947924 |
| Cathepsin B | 1.4048829 | 1.2978196 | 1.2959604 | 1.0602698 | 1.0057857 | 1.1033943 |
| Phase-1 RCT-144 | 0.911614 | 0.9085678 | 0.7890953 | 0.9088324 | 0.898292 | 0.876052 |
| Alpha-1 microglobulin/bikunin precursor (Ambp) | 1.2573711 | 1.2025913 | 1.1508174 | 1.1323805 | 1.0716091 | 1.1059785 |
| Cyclin D1 | 0.9464434 | 1.0208777 | 0.7306938 | 0.855703 | 1.0732175 | 1.0045903 |
| Presenilin-1 | 0.8349696 | 0.8037624 | 1.121465 | 1.3487341 | 1.3671846 | 1.6418871 |
| Protein tyrosine phosphatase, receptor type, D | 1.4611385 | 1.3873389 | 1.4464346 | 1.1396631 | 1.367607 | 1.3445079 |
| Multidrug resistant protein-1 | 1.6613973 | 1.5027198 | 1.1022202 | 0.8598121 | 0.95925 | 0.9147982 |
| Cathepsin L, sequence 2 | 1.3517692 | 1.5481447 | 1.3716366 | 1.1134899 | 1.2533569 | 1.2442148 |
| JNK1 stress activated protein kinase | 1.0954101 | 1.4085459 | 1.2513661 | 1.6244599 | 1.0716865 | 0.9597351 |
| Phase-1 RCT-43 | 0.8136633 | 0.8505719 | 0.7161115 | 1.101656 | 0.9765313 | 1.0533748 |
| Tissue plasminogen activator | 0.9473354 | 0.9554184 | 0.9247072 | 1.0317382 | 1.0074824 | 1.0572953 |
| Ubiquitin conjugating enzyme (RAD 6 | 1.0478432 | 1.133723 | 1.1227317 | 0.9539966 | 1.2037276 | 0.9841311 |
| homologue) | | | | | | |
| Phase-1 RCT-173 | 0.8552747 | 1.04109 | 1.1096245 | 1.0894074 | 0.9952132 | 0.8990522 |
| Ornithine decarboxylase | 0.9781167 | 1.0938909 | 1.0299417 | 0.9100559 | 0.6445763 | 0.6267481 |
| Zinc finger protein | 0.8048567 | 0.9401031 | 0.8767277 | 0.9413831 | 0.9504716 | 0.9127374 |
| CDK108 | 0.6040078 | 0.8489295 | 0.533524 | 1.202533 | 0.8768492 | 0.9276943 |
| Phase-1 RCT-117 | 1.0459871 | 1.2959243 | 1.1046057 | 0.9511775 | 1.0672834 | 0.9957365 |
| {Phase-1 RCT 98} | 0.8925944 | 0.7555147 | 0.7926617 | 0.9934315 | 1.0553612 | 1.0075483 |
| Aquaporin-3 (AQP3) | 0.7883652 | 0.7020149 | 0.7018076 | 0.9451975 | 0.9611135 | 0.8962692 |
| Cholesterol esterase | 1.3484248 | 2.1885068 | 1.2761889 | 1.4131219 | 1.1587989 | 1.1685419 |
| Ribosomal protein S17 | 1.3764111 | 1.3232287 | 1.3071564 | 1.1794089 | 1.236496 | 1.2928337 |
| Poly(ADP-ribose) polymerase | 1.028819 | 0.8772701 | 1.7006365 | 0.857888 | 0.8400675 | 0.7885719 |
| Annexin V | 1.0325953 | 1.1029941 | 1.0554248 | 0.921005 | 1.118881 | 1.0234855 |
| Phase-1 RCT-33 | 1.3428836 | 0.8911326 | 1.2404907 | 1.088951 | 1.0025655 | 1.0889592 |
| Phase-1 RCT-61 | 0.7554209 | 0.9134603 | 0.653396 | 0.8933116 | 1.2272693 | 0.9729959 |
| Retinol dehydrogenase type III | 1.0993906 | 1.3712003 | 1.273974 | 1.3085166 | 1.3334291 | 1.1298102 |
| ATPase inhibitor (rat mitochondrial IF1 protein) | 1.1035111 | 1.420688 | 1.1398587 | 1.349404 | 1.367724 | 1.3739301 |
| Thymidylate synthase | 1.0587908 | 1.1179694 | 1.1311139 | 0.7980844 | 1.0377074 | 0.9753623 |
| Interleukin-18 | 0.9601317 | 1.1074477 | 0.9335281 | 0.9303798 | 0.9704309 | 0.8557349 |
| Lecithin: cholesterol acyltransferase | 1.2743626 | 1.2943007 | 1.2492023 | 1.0388643 | 1.0205555 | 0.996709 |
| Contrapsin-like protease inhibitor (CPi-21) | 1.4712206 | 1.5308656 | 1.725673 | 1.3785425 | 0.9812952 | 1.1079063 |
| Proliferating cell nuclear antigen gene | 0.9643259 | 1.559482 | 0.8370611 | 0.8508205 | 0.9220238 | 0.8373885 |
| Phase-1 RCT-230 | 0.762079 | 1.0144832 | 0.7630585 | 0.8452615 | 0.9075408 | 0.8329161 |
| Cytochrome P450 2D18 | 1.0011578 | 0.9925089 | 1.0810784 | 1.0826889 | 0.9004377 | 0.8455306 |
| Phase-1 RCT-48 | 0.8840383 | 0.9467446 | 0.96962 | 1.2201039 | 1.0325528 | 0.9955943 |
| Phase-1 RCT-292 | 0.7682573 | 0.8790797 | 0.8245135 | 0.9810396 | 0.9747865 | 0.9435279 |
| Arginosuccinate synthetase 1 | 1.0639672 | 0.5634851 | 0.9524351 | 1.1362301 | 1.013992 | 0.8305368 |
| C-reactive protein | 1.2633083 | 1.2968726 | 1.2973043 | 1.0583336 | 1.360634 | 1.2997134 |

| | BEN | BEN | BEN | BRB | BRB | BRB | BRB |
|---|---|---|---|---|---|---|---|
| Compound-Dose | 1000 | 1000 | 1000 | 200 | 200 | 200 | 800 |
| Animal Number | 2037 | 2038 | 2039 | 2327 | 2328 | 2329 | 2337 |
| Liver Toxicity Necrosis Classification | no | no | no | yes | yes | yes | yes |
| Gene Name | | | | | | | |
| Osteoactivin | 1.7714854 | 1.1497769 | 1.0558296 | 2.9468195 | 3.9033034 | 4.8531466 | 3.257925 |
| Stathmin | 0.8915386 | 0.9986449 | 0.6386228 | 1.1402411 | 2.3607128 | 2.978854 | 2.0600462 |
| Calpactin I heavy chain | 1.2956395 | 1.0305443 | 1.0846181 | 1.0865892 | 1.8334358 | 2.4371116 | 1.8536006 |
| Phase-1 RCT-179 | 0.9399814 | 1.025312 | 0.8118 | 1.6835997 | 1.6269447 | 1.9696738 | 1.6219666 |
| Phase-1 RCT-107 | 0.9051704 | 1.043155 | 0.9341176 | 0.7727622 | 0.6237829 | 0.5761005 | 0.6885424 |
| Heme oxygenase | 1.0298542 | 0.9250947 | 0.9826153 | 1.9793903 | 1.7488586 | 2.5362668 | 1.6420211 |
| Collagen type II | 0.9207693 | 1.1440945 | 0.9412994 | 1.4732435 | 2.2941186 | 3.6570728 | 2.8178325 |
| Phase-1 RCT-81 | 1.1467057 | 0.7652832 | 1.2923003 | 0.7905642 | 0.7622994 | 0.8134939 | 0.7978592 |
| Glycine methyltransferase | 0.9673993 | 1.0206501 | 1.054314 | 1.0737413 | 0.7855302 | 0.4257802 | 0.544453 |
| Cystatin C | 1.2014495 | 0.7604463 | 1.1038046 | 1.5611454 | 1.6873826 | 1.9346591 | 1.8024365 |
| Melanoma-associated antigen ME491 | 1.2160808 | 1.0265638 | 1.203987 | 1.0846868 | 1.0906752 | 2.025034 | 1.5277342 |
| Phase-1 RCT-146 | 0.8982046 | 1.1127162 | 0.8616027 | 0.8461375 | 1.321227 | 1.3249532 | 1.2866919 |
| Gamma-actin, cytoplasmic | 1.0632156 | 0.9851142 | 0.9421686 | 0.9215995 | 1.9852321 | 2.9552138 | 1.6954335 |
| Adenine nucleotide translocator 1 | 0.9068423 | 1.1356832 | 0.8251789 | 1.2862234 | 1.3214592 | 1.4810405 | 1.4063314 |
| Thymosin beta-10 | 1.427014 | 0.8808691 | 1.3001493 | 1.7798431 | 3.3040884 | 3.8144536 | 2.9910321 |
| Insulin-like growth factor I, exon 6 | 0.9477416 | 1.0110703 | 0.9557478 | 0.73034 | 0.8332628 | 0.7113897 | 0.6462033 |
| Phase-1 RCT-78 | 1.1548288 | 0.8828036 | 1.0025762 | 0.6580538 | 0.7599592 | 0.7757164 | 0.8465018 |
| Alpha-2-microglobulin | 1.031366 | 0.5050429 | 0.7053277 | 0.2972899 | 0.5034348 | 0.4240656 | 0.1497334 |
| High affinity IgE receptor gamma chain | 1.3379341 | 1.0103543 | 1.1260664 | 1.3287559 | 1.4440914 | 1.9006971 | 1.7916274 |
| (FcERIgamma) | | | | | | | |
| 60S ribosomal protein L6 | 1.0412484 | 0.6530127 | 1.114041 | 1.284749 | 1.6086637 | 1.818548 | 1.5489585 |
| IgE binding protein | 1.1541301 | 1.0463545 | 0.8834909 | 1.4015443 | 2.0311606 | 3.1680524 | 2.0825603 |
| Phase-1 RCT-149 | 0.9768735 | 0.9579175 | 1.0032585 | 0.8241898 | 0.7285345 | 0.8057048 | 0.7796985 |
| Cytochrome P450 2C11 | 1.1316131 | 1.3520124 | 1.0466636 | 0.6964477 | 0.6088088 | 1.3836515 | 0.6955023 |
| Uncoupling protein 2 | 1.5153452 | 0.9415544 | 1.216734 | 1.4126846 | 1.7535679 | 2.5119882 | 1.9007322 |
| Cofilin | 1.0930597 | 0.944095 | 0.9886786 | 1.4748895 | 2.1497068 | 2.6273787 | 2.1145723 |
| Alpha-tubulin | 0.871197 | 1.0128803 | 0.7999808 | 0.7604177 | 2.305954 | 2.4801142 | 1.9489945 |
| Stearyl-CoA desaturase, liver | 0.8236754 | 1.0240332 | 0.9324352 | 0.0492857 | 0.6051916 | 0.3736922 | 0.3531312 |
| Glutathione S-transferase theta-1 | 0.9234102 | 0.7995516 | 1.0906599 | 1.4082462 | 1.5809367 | 1.3445275 | 1.6849217 |
| alpha-1,2-fucosyltrans-ferase | 1.0693065 | 0.7256504 | 0.899199 | 0.6922385 | 0.8412322 | 0.8879147 | 0.9541515 |
| Phase-1 RCT-138 | 1.2864752 | 1.0545361 | 1.1956809 | 1.4220799 | 1.5587143 | 1.683029 | 1.5988415 |
| Phase-1 RCT-295 | 1.3520672 | 0.7600319 | 1.2617129 | 1.412557 | 2.0661442 | 2.4332964 | 2.1740801 |
| Phase-1 RCT-164 | 0.8109757 | 0.9781462 | 0.915655 | 0.7173139 | 0.6097868 | 0.6459987 | 0.6484931 |
| Alpha-2-macroglobulin, sequence 2 | 1.0082139 | 1.0985571 | 1.0303004 | 1.5756878 | 1.1807061 | 1.350547 | 1.1978787 |
| Biliverdin reductase | 1.0093299 | 1.1575806 | 1.0073342 | 1.4558543 | 1.4956446 | 1.4730015 | 1.7364639 |
| Phase-1 RCT-121 | 0.8520106 | 1.0747252 | 0.7157901 | 1.337518 | 1.272791 | 1.4043186 | 1.4291171 |
| Dynamin-1 (D100) | 0.8340156 | 1.120753 | 0.8736473 | 0.8103132 | 0.6855177 | 0.7597103 | 0.7937414 |
| Phase-1 RCT-192 | 1.2385508 | 1.1221333 | 1.0816047 | 1.2644708 | 1.4451277 | 2.895795 | 1.61397 |
| Fatty acid synthase | 0.7042397 | 1.0425545 | 1.0364006 | 0.1859671 | 0.4651857 | 0.353178 | 0.3842214 |
| Phase-1 RCT-211 | 1.0619961 | 0.9159868 | 1.145995 | 0.8006349 | 1.3717129 | 1.5896095 | 1.6801641 |
| Ribosomal protein S9 | 0.8234019 | 0.72817 | 0.973701 | 1.1485629 | 1.7010472 | 1.8365332 | 1.8691975 |
| NADP-dependent isocitrate dehydrogenase, | 0.9017463 | 1.0899587 | 0.9205411 | 1.2810172 | 1.7259021 | 1.9716454 | 1.8209739 |
| cytosolic | | | | | | | |
| Phase-1 RCT-57 | 0.9733607 | 0.8966882 | 0.8649588 | 0.7150968 | 0.6277614 | 0.7460676 | 0.730805 |
| Phase-1 RCT-36 | 0.9194362 | 0.9656686 | 0.9302421 | 0.7215163 | 0.6488755 | 0.930272 | 0.9483573 |
| Apolipoprotein All | 0.7038816 | 0.7520074 | 0.8334872 | 0.3512048 | 0.6614288 | 0.2640552 | 0.5814197 |
| Phase-1 RCT-68 | 1.135655 | 0.9714704 | 0.9283909 | 0.9594756 | 1.0604378 | 1.2058328 | 1.2584047 |
| Multidrug resistant protein-2 | 0.9974933 | 1.0645893 | 0.9895806 | 1.2993512 | 1.3384098 | 2.095334 | 1.3810167 |
| Phase-1 RCT-39 | 1.1829119 | 1.0589617 | 1.0316714 | 0.8869088 | 0.9529468 | 1.098874 | 1.022172 |
| NIPK | 1.0084617 | 1.0766658 | 0.9008725 | 1.2783798 | 0.6970967 | 0.7158685 | 0.7773125 |
| F1-ATPase beta subunit | 1.0308927 | 0.9139375 | 1.248208 | 1.7148339 | 1.6371404 | 1.8557724 | 2.0506713 |
| Elongation factor-1 alpha | 1.2092633 | 0.859879 | 1.5513185 | 1.5673044 | 1.746723 | 2.0661118 | 2.2336087 |
| Phase-1 RCT-24 | 0.9134617 | 1.1194144 | 0.9262558 | 0.7596304 | 1.9745862 | 2.4987066 | 2.022376 |
| Beta-tubulin, class I | 1.2312961 | 0.9741628 | 1.1584315 | 1.0018606 | 4.114321 | 3.307786 | 3.0904756 |
| 14-3-3 zeta | 0.8506739 | 1.1577379 | 0.8655887 | 1.403593 | 1.3697712 | 2.007074 | 1.7307416 |
| Alpha-prothymosin | 1.2757922 | 0.7121499 | 1.1358895 | 1.1481184 | 2.3500555 | 2.059401 | 1.8918374 |
| Phase-1 RCT-58 | 0.8674049 | 1.1257592 | 0.8834993 | 2.8592405 | 2.3212914 | 2.439464 | 3.7249212 |
| Phase-1 RCT-290 | 0.7609485 | 0.8863219 | 1.0634284 | 0.6416025 | 0.7658461 | 0.5882461 | 0.9213471 |
| Transitional endoplasmic reticulum ATPase | 0.899022 | 0.9857353 | 0.8125947 | 1.3247716 | 0.983288 | 1.1367193 | 1.0257447 |
| Beta-actin | 0.9867574 | 1.0856657 | 0.9177255 | 1 | 1.8412466 | 2.7554574 | 2.2243006 |
| Thioredoxin-1 (Trx1) | 1.1278694 | 0.7612621 | 1.245626 | 0.8313722 | 1.453829 | 1.5194999 | 1.663105 |
| Phase-1 RCT-174 | 1.093979 | 0.853941 | 1.1628745 | 0.8152441 | 0.7001238 | 0.7449531 | 0.7619063 |
| Beta-actin, sequence 2 | 1.2104832 | 0.7723189 | 1.0436871 | 1.1627727 | 1.8711132 | 2.1258667 | 1.8288682 |
| Phase-1 RCT-109 | 1.0627174 | 0.7249818 | 1.215206 | 1.0776913 | 1.5906646 | 2.0808203 | 1.798432 |
| Phase-1 RCT-154 | 0.9251086 | 1.0520064 | 1.0029156 | 1.0160043 | 1.1351324 | 1.4523622 | 1.2609577 |
| Hypoxanthine-guanine | 0.9767149 | 0.6862088 | 1.0672741 | 1.0172098 | 1.5020621 | 1.8090909 | 1.695081 |
| phosphoribosyltransferase | | | | | | | |
| Phase-1 RCT-213 | 1.0037698 | 0.8968927 | 0.9267806 | 0.9790334 | 1.073699 | 1.2157297 | 1.1869386 |
| Phase-1 RCT-260 | 1.1549801 | 0.9426817 | 1.0401055 | 0.7441521 | 0.7266741 | 0.6586147 | 0.7439498 |
| Phase-1 RCT-12 | 1.0052476 | 1.0988791 | 1.0571955 | 1.2127181 | 1.053487 | 1.7814792 | 1.6303462 |
| Phase-1 RCT-293 | 1.7621447 | 1.1750623 | 1.4691037 | 1.0774947 | 1.5204513 | 2.0537834 | 1.5671492 |
| Ribosomal protein S8 | 1.2672893 | 0.8619207 | 1.4808013 | 1.54954 | 2.1811929 | 2.391112 | 2.0316868 |
| Pyruvate kinase, muscle | 1.5133659 | 1.0757774 | 0.9967832 | 0.8975138 | 1.3203523 | 1.4429157 | 1.2895952 |
| Nucleoside diphosphate kinase beta isoform | 1.0298749 | 0.9143966 | 1.2315038 | 1.2472756 | 1.8499653 | 2.7569854 | 2.1220007 |
| p55CDC | 0.8491539 | 1.1898832 | 0.8275939 | 1.2174098 | 2.2936676 | 1.5719229 | 1.5075492 |
| Phase-1 RCT-156 | 1.0477419 | 0.842767 | 1.0537775 | 1.233008 | 1.28778 | 1.3806046 | 1.252535 |
| Ribosomal protein L13A | 1.1871866 | 0.6693688 | 1.3814647 | 1.2385345 | 1.9794277 | 2.3379884 | 2.0492268 |
| Phase-1 RCT-258 | 1.0129975 | 0.9013203 | 0.9016503 | 0.9074028 | 0.8561604 | 1.0267221 | 0.9839225 |
| Insulin-like growth factor I | 1.0847634 | 0.5040838 | 1.2665367 | 0.7411249 | 0.8949658 | 0.7737891 | 0.726454 |
| Cytochrome P450 3A1 | 0.9971038 | 0.9499345 | 0.9762051 | 0.466176 | 0.9329636 | 0.4985851 | 0.8332987 |
| {Ribosomal protein 16} | 1.191235 | 0.8703739 | 1.3836125 | 1.4302766 | 2.0540235 | 2.0567942 | 1.8685595 |
| Organic cation transporter 3 | 0.9871021 | 0.779264 | 1.3326048 | 1.0441613 | 1.0820467 | 1.5866146 | 1.0697105 |
| Calpain 2 | 1 | 0.988207 | 0.8894072 | 0.9937571 | 1.0503534 | 1.3379524 | 1.073485 |
| Phase-1 RCT-102 | 0.8641087 | 1.1920191 | 0.8887535 | 0.5474128 | 0.6616263 | 0.4557648 | 0.4088608 |
| Equilbrative nitrobenzylthioinosine-sensitive | 1.2540687 | 0.9631533 | 1.1441816 | 0.8780341 | 0.8772436 | 0.7735068 | 0.7971839 |
| nucleoside transporter | | | | | | | |
| Phase-1 RCT-227 | 1.3205866 | 0.7479386 | 1.3931638 | 1.1002557 | 1.1372662 | 0.6574865 | 0.6973003 |
| N-hydroxy-2-acetylaminofluorene sulfotransferase (ST1C1) | 0.9928929 | 1.0045737 | 1.0563786 | 0.768902 | 0.8297927 | 0.6260173 | 0.7041129 |
| Cathepsin S | 1.1647305 | 1.0746688 | 0.9238492 | 1.8619932 | 1.485594 | 1.5859995 | 1.3345643 |
| Apolipoprotein CIII | 0.9225798 | 0.6180133 | 1.1737684 | 0.7216275 | 0.7254869 | 0.5979998 | 0.5359058 |
| Cytochrome P450 2A3 | 0.9694154 | 1.4075181 | 0.9996356 | 1.4222832 | 0.5872005 | 0.3663773 | 0.5230479 |
| Cytochrome P450 2C23 | 0.8115698 | 0.9070762 | 0.9272316 | 1.6615431 | 1.7709253 | 0.7873204 | 1.2205002 |
| Apolipoprotein C1 | 1.1854701 | 0.671711 | 1.760457 | 1.3161744 | 1.043584 | 0.7207968 | 0.6545721 |
| Betaine homocysteine methyltransferase (BHMT) | 0.7105645 | 0.8121645 | 1.2323554 | 0.5683478 | 1.2205142 | 0.4922842 | 1.060364 |
| Paraoxonase 1 | 0.8364637 | 0.8558648 | 1.0779369 | 1.5912638 | 1.0961878 | 0.9235965 | 0.8918986 |
| Phase-1 RCT-207 | 0.8596423 | 1.1037924 | 0.7383014 | 0.8862279 | 0.9453741 | 1.1838957 | 1.1102386 |
| Cathepsin B | 1.2638927 | 0.8411813 | 1.5163757 | 1.4823563 | 1.7322345 | 1.7976509 | 1.862167 |
| Phase-1 RCT-144 | 0.8681541 | 1.143697 | 0.9131672 | 0.9922551 | 0.8415777 | 1.0370655 | 1.0904684 |
| Alpha-1 microglobulin/bikunin precursor (Ambp) | 1.1911471 | 0.6508022 | 1.5434134 | 1.2708362 | 1.3203362 | 0.9021991 | 0.9105306 |
| Cyclin D1 | 0.9222618 | 1.1631396 | 0.9772311 | 1.0805936 | 1.1734641 | 1.2697256 | 1.567103 |
| Presenilin-1 | 1.3544774 | 0.7770805 | 1.1829492 | 1.2526206 | 0.7326231 | 0.5660425 | 0.6804338 |
| Protein tyrosine phosphatase, receptor type, D | 1.5792198 | 0.8281282 | 1.5040833 | 1.0379419 | 0.8362327 | 0.5895201 | 0.7711018 |
| Multidrug resistant protein-1 | 0.997781 | 1.1051728 | 1.0062608 | 1.4743623 | 1.3101763 | 2.0496535 | 1.5871942 |
| Cathepsin L, sequence 2 | 0.6880428 | 0.8941622 | 1.0060538 | 2.088933 | 1.5473577 | 1.9024348 | 1.9036177 |
| JNK1 stress activated protein kinase | 0.9803923 | 1.0710346 | 0.9190275 | 0.833959 | 0.79093 | 0.5981025 | 0.7460544 |
| Phase-1 RCT-43 | 0.8396744 | 1.1015825 | 0.8439261 | 1.0629795 | 1.1265796 | 1.1300827 | 1.1315527 |
| Tissue plasminogen activator | 0.9608048 | 0.9879285 | 0.9127719 | 0.7143318 | 0.9607958 | 1.2609117 | 1.3524277 |
| Ubiquitin conjugating enzyme (RAD 6 | 0.7871512 | 0.8152418 | 0.8822445 | 0.9145531 | 0.9966877 | 1.5702314 | 0.8497846 |
| homologue) | | | | | | | |
| Phase-1 RCT-173 | 0.8185605 | 1.0346228 | 0.780804 | 0.9662333 | 1.2572962 | 1.3455532 | 1.6250732 |
| Ornithine decarboxylase | 0.9477514 | 1.0504317 | 0.8136054 | 0.9052507 | 1.2398201 | 1.4360931 | 1.2974095 |
| Zinc finger protein | 0.9846932 | 1.0257689 | 0.8415464 | 0.9535018 | 1.0057323 | 0.9653457 | 0.9935393 |
| CDK108 | 0.9503787 | 0.9254795 | 0.850606 | 0.62216 | 0.6018497 | 0.7659586 | 0.7301804 |
| Phase-1 RCT-117 | 1.3768348 | 0.9247196 | 1.3994169 | 0.7237371 | 0.6941465 | 0.6631166 | 0.826264 |
| {Phase-1 RCT 98} | 0.9225458 | 0.8668 | 0.8476756 | 0.7185122 | 0.637303 | 0.7663073 | 0.7439179 |
| Aquaporin-3 (AQP3) | 0.9130583 | 1.0244367 | 0.8931972 | 0.7178769 | 0.6389068 | 0.7386072 | 0.7129144 |
| Cholesterol esterase | 0.661257 | 0.8720049 | 0.6955925 | 0.6193295 | 0.9308044 | 0.5596929 | 0.5066437 |
| Ribosomal protein S17 | 1.2848841 | 0.8952596 | 1.2724836 | 1.3301822 | 1.8066574 | 2.2792068 | 1.8467163 |
| Poly(ADP-ribose) polymerase | 0.8329671 | 1.1241857 | 0.8571892 | 1.0052885 | 1.049634 | 1.11052 | 1.2370495 |
| Annexin V | 0.9026006 | 1.0185215 | 1.0519643 | 1.011234 | 1.3029742 | 1.6915519 | 1.11355 |
| Phase-1 RCT-33 | 1.0092535 | 0.9252423 | 1.0794586 | 0.6658546 | 0.9914093 | 1.1227336 | 1.2551826 |
| Phase-1 RCT-61 | 0.8753344 | 1.1029555 | 0.8654484 | 0.9193109 | 0.7667397 | 0.9801942 | 0.993116 |
| Retinol dehydrogenase type III | 1.1026949 | 0.6522998 | 1.0760951 | 1.060804 | 1.1599647 | 0.8834291 | 0.8571855 |
| ATPase inhibitor (rat mitochondrial IF1 protein) | 0.7936943 | 0.7348653 | 0.9474369 | 0.7467454 | 1.1967664 | 0.861966 | 0.6824868 |
| Thymidylate synthase | 0.9183242 | 1.1489766 | 0.8080981 | 1.3184234 | 1.0901914 | 1.1578175 | 0.9488055 |
| Interleukin-18 | 0.8832526 | 10.511533 | 0.943734 | 0.9622389 | 0.857473 | 0.7411448 | 0.6822019 |
| Lecithin: cholesterol acyltransferase | 1.2841854 | 0.6714763 | 1.6467121 | 1.3141036 | 1.1537783 | 1.1779335 | 1.1241007 |
| Contrapsin-like protease inhibitor (CPi-21) | 1.0213906 | 0.5793659 | 0.8101082 | 1.2026023 | 1.0287324 | 0.8965508 | 0.7617999 |
| Proliferating cell nuclear antigen gene | 0.9487455 | 1.1877397 | 0.9205411 | 0.8061791 | 0.9406393 | 0.8990107 | 1.2327284 |
| Phase-1 RCT-230 | 1.1829228 | 1.0396498 | 1.116549 | 0.7059123 | 0.8481833 | 0.9162688 | 0.96313 |
| Cytochrome P450 2D18 | 1.2508193 | 0.7130977 | 1.4035865 | 1.3248413 | 1.1307435 | 0.8324607 | 0.7757452 |
| Phase-1 RCT-48 | 1.1674159 | 0.7669359 | 1.3654498 | 0.7806346 | 0.9738709 | 0.8846359 | 0.9067848 |
| Phase-1 RCT-292 | 0.9146635 | 1.0942997 | 0.9529955 | 0.9158368 | 0.7816947 | 0.8858844 | 0.8554177 |
| Arginosuccinate synthetase 1 | 0.8214243 | 0.7224103 | 1.2443393 | 1.1917964 | 1.0730677 | 0.592599 | 0.9641157 |
| C-reactive protein | 1.5009211 | 0.6969331 | 1.4725935 | 1.1384834 | 0.9096397 | 0.7258303 | 0.8542328 |

| | BRB | BRB | BUS | BUS | BUS | CAD |
|---|---|---|---|---|---|---|
| Compound-Dose | 800 | 800 | 14 | 14 | 14 | 1 |
| Animal Number | 2338 | 2339 | 1747 | 1748 | 1749 | 627 |
| Liver Toxicity Necrosis Classification | yes | yes | no | no | no | no |
| Gene Name | | | | | | |
| Osteoactivin | 4.552991 | 11.614666 | 0.7743083 | 0.7261053 | 0.998427 | 1.099122 |
| Stathmin | 1.7306098 | 2.7680163 | 0.8251013 | 0.9204439 | 0.8018185 | 0.8864513 |
| Calpactin I heavy chain | 2.5859466 | 3.5943158 | 1.0717795 | 1 | 0.984588 | 1.086534 |
| Phase-1 RCT-179 | 2.3027236 | 3.0223055 | 1.1755471 | 1.4347752 | 1.0461354 | 0.9101587 |
| Phase-1 RCT-107 | 0.5958927 | 0.6449599 | 0.770788 | 0.8266852 | 0.907684 | 0.9413157 |
| Heme oxygenase | 2.666611 | 2.9055958 | 0.8397486 | 0.9217719 | 1.1511713 | 0.9859363 |
| Collagen type II | 1.3447584 | 1.7479692 | 1.5536934 | 0.970741 | 1.0920753 | 0.9623622 |
| Phase-1 RCT-81 | 0.7992837 | 0.6909981 | 0.8179026 | 0.9027453 | 0.9657707 | 0.9386835 |
| Glycine methyltransferase | 0.5367469 | 0.3766622 | 0.9854073 | 0.7391834 | 1.3488361 | 0.7331911 |
| Cystatin C | 2.060065 | 1.8302889 | 1.1238804 | 1.1496592 | 1.0474608 | 1.1464726 |
| Melanoma-associated antigen ME491 | 1.9358548 | 1.9230475 | 0.8105096 | 0.7377471 | 0.9421309 | 0.9953169 |
| Phase-1 RCT-146 | 1.4085838 | 2.5591617 | 0.7054467 | 0.6956933 | 0.7420304 | 1.0875362 |
| Gamma-actin, cytoplasmic | 3.6883225 | 4.009283 | 1.0442452 | 1.1056265 | 0.9532268 | 0.8148831 |
| Adenine nucleotide translocator 1 | 1.4787759 | 1.8118958 | 1.236126 | 1.151369 | 0.9995523 | 0.8711906 |
| Thymosin beta-10 | 2.3344657 | 4.4736185 | 1.0808729 | 0.9103546 | 1.0238069 | 1.0364082 |
| Insulin-like growth factor I, exon 6 | 0.6569049 | 0.4292359 | 1.195696 | 1.2506033 | 0.9919701 | 0.7582827 |
| Phase-1 RCT-78 | 0.738811 | 0.7616694 | 0.9907997 | 1.0171007 | 1.1670578 | 0.9453382 |
| Alpha-2-microglobulin | 0.2139301 | 0.150329 | 0.7693655 | 0.879508 | 0.6975294 | 0.7015319 |
| High affinity IgE receptor gamma chain | 2.033609 | 2.571395 | 0.8261297 | 0.8823861 | 1.1226264 | 1.108287 |
| (FcERIgamma) | | | | | | |
| 60S ribosomal protein L6 | 2.3567622 | 2.6756253 | 1.465312 | 1.1800829 | 1.0979178 | 1.0292033 |
| IgE binding protein | 3.3272195 | 7.3242702 | 0.8515394 | 0.812272 | 1.0021703 | 1.2858903 |
| Phase-1 RCT-149 | 0.750516 | 0.6838148 | 0.9168378 | 0.9100967 | 0.971179 | 0.9823721 |
| Cytochrome P450 2C11 | 0.5906424 | 0.6571104 | 0.8534187 | 0.5044493 | 0.7667325 | 0.5621371 |
| Uncoupling protein 2 | 1.6812453 | 2.6750073 | 0.8345643 | 0.9571495 | 1.2343676 | 1.0536292 |
| Cofilin | 3.01397 | 3.709798 | 1.4247706 | 1.159303 | 1.1644197 | 1.1154919 |
| Alpha-tubulin | 2.6189246 | 3.599393 | 1.4341286 | 1.4761744 | 1.1045039 | 1.0390459 |
| Stearyl-CoA desaturase, liver | 0.1271243 | 0.0911533 | 0.6746129 | 0.2577314 | 1.4722239 | 1.5915726 |
| Glutathione S-transferase theta-1 | 1.3219473 | 1.5524569 | 1.2949823 | 1.3989816 | 1.0915068 | 1.0747991 |
| alpha-1,2-fucosyltrans-ferase | 0.8456576 | 0.9843731 | 1.0997162 | 1.2339101 | 0.9960115 | 0.9771624 |
| Phase-1 RCT-138 | 2.1085773 | 2.28398 | 0.9424908 | 0.9150881 | 1.0282143 | 1.0192845 |
| Phase-1 RCT-295 | 2.5925894 | 3.4402854 | 1.2612063 | 1.172805 | 1.188067 | 1.0741417 |
| Phase-1 RCT-164 | 0.6598213 | 0.6022717 | 0.8264899 | 0.726209 | 0.8251646 | 1.0054892 |
| Alpha-2-macroglobulin, sequence 2 | 1.2064518 | 1.2711252 | 0.8522139 | 0.9353041 | 1.0009278 | 1.2979425 |
| Biliverdin reductase | 1.0909985 | 2.4420905 | 1.094426 | 0.8688998 | 1.0343641 | 1.0892258 |
| Phase-1 RCT-121 | 1.2234522 | 2.0220513 | 0.7969895 | 0.9928327 | 0.9617955 | 1.0431366 |
| Dynamin-1 (D100) | 0.7240465 | 0.6383119 | 0.7231427 | 0.6616621 | 0.8203214 | 0.9588938 |
| Phase-1 RCT-192 | 2.4729211 | 2.4745862 | 1.0307496 | 1.0725373 | 0.8328632 | 1.2242922 |
| Fatty acid synthase | 0.3727616 | 0.292801 | 0.6792075 | 0.6444012 | 1.32387 | 1.3030732 |
| Phase-1 RCT-211 | 1.6707944 | 1.9827275 | 1.0421301 | 1.0122898 | 1.045917 | 1.0334004 |
| Ribosomal protein S9 | 1.6685059 | 2.45391 | 1.3643243 | 1.0726833 | 0.9382306 | 1.1315293 |
| NADP-dependent isocitrate dehydrogenase, | 2.2309659 | 1.930493 | 1.5212191 | 1.3597091 | 1.0732509 | 0.9363384 |
| cytosolic | | | | | | |
| Phase-1 RCT-57 | 0.702263 | 0.7128446 | 0.9047353 | 0.8966242 | 0.8473314 | 1.0168868 |
| Phase-1 RCT-36 | 0.9798139 | 0.8430369 | 0.8901464 | 0.9630426 | 1.0808079 | 0.9641393 |
| Apolipoprotein All | 0.5187933 | 0.427041 | 0.8286327 | 0.808896 | 1.1043884 | 0.7722161 |
| Phase-1 RCT-68 | 1.3107048 | 1.8542246 | 0.9306436 | 0.8852686 | 1.0042715 | 1.0341536 |
| Multidrug resistant protein-2 | 1.2554348 | 2.5788338 | 1.4572119 | 1.3279049 | 1.2645026 | 0.8756045 |
| Phase-1 RCT-39 | 1.2931995 | 1.2333636 | 0.995342 | 1.0918008 | 0.9819685 | 1.0749216 |
| NIPK | 0.6406918 | 0.7746794 | 0.7403993 | 0.9730421 | 0.9560815 | 0.9486538 |
| F1-ATPase beta subunit | 2.1249478 | 1.6537527 | 1.2011381 | 1.2717996 | 1.134783 | 0.9798573 |
| Elongation factor-1 alpha | 2.9921114 | 2.6970434 | 1.3058046 | 1.2146926 | 1.0395337 | 0.9572727 |
| Phase-1 RCT-24 | 3.84797 | 4.150613 | 1.3147074 | 1.3208374 | 1.1176499 | 1.1323692 |
| Beta-tubulin, class I | 4.762975 | 6.199554 | 1.5409166 | 1.5389569 | 1.0206902 | 1.1128898 |
| 14-3-3 zeta | 1.9510972 | 2.868237 | 1.3925308 | 1.2182678 | 1.2669629 | 0.9000934 |
| Alpha-prothymosin | 2.8168075 | 3.0613465 | 1.2243212 | 0.8547036 | 0.7844808 | 0.8001834 |
| Phase-1 RCT-58 | 2.5047328 | 1.9916269 | 1.8510907 | 2.241978 | 0.859075 | 1.1232406 |
| Phase-1 RCT-290 | 0.6521255 | 0.5464998 | 0.8000321 | 0.755285 | 1.0645941 | 1.1585186 |
| Transitional endoplasmic reticulum ATPase | 1.4501932 | 1.3989881 | 1.3378503 | 1.290935 | 1.1471248 | 0.9614236 |
| Beta-actin | 2.7534175 | 3.9160047 | 1.0993712 | 1.1594763 | 0.9955575 | 0.9290886 |
| Thioredoxin-1 (Trx1) | 2.4259205 | 2.3662174 | 1.0037292 | 1.297969 | 1.0987141 | 1.1321508 |
| Phase-1 RCT-174 | 0.7413985 | 0.7902284 | 0.7535244 | 0.7508268 | 0.8148687 | 0.9665527 |
| Beta-actin, sequence 2 | 2.6355596 | 3.1734638 | 1.1667067 | 1.3137206 | 1.2610158 | 1.1085948 |
| Phase-1 RCT-109 | 2.5756073 | 2.8308856 | 1.247419 | 1.0086683 | 1.2070813 | 1.0147148 |
| Phase-1 RCT-154 | 1.6232723 | 2.010598 | 1.0623173 | 0.9251892 | 1.0504011 | 1.09756 |
| Hypoxanthine-guanine | 1.9576074 | 2.0741048 | 1.2945741 | 0.9518978 | 1.099178 | 1.0529081 |
| phosphoribosyltransferase | | | | | | |
| Phase-1 RCT-213 | 1.0729957 | 1.4959016 | 0.9568179 | 1.0760739 | 0.9419831 | 1.1454931 |
| Phase-1 RCT-260 | 0.5689204 | 0.5985763 | 0.7440004 | 0.7917618 | 0.7492576 | 1.0670183 |
| Phase-1 RCT-12 | 2.1883473 | 1.8576539 | 0.8521197 | 1.1397407 | 1.1007844 | 0.9501388 |
| Phase-1 RCT-293 | 1.5731025 | 2.5988476 | 0.7826382 | 0.7685795 | 0.8676202 | 0.9427074 |
| Ribosomal protein S8 | 3.622021 | 3.298959 | 1.2131903 | 1.0241473 | 1.0224441 | 1.1942743 |
| Pyruvate kinase, muscle | 1.3393977 | 2.6244667 | 0.8705793 | 0.8925622 | 1.1804616 | 1.0721862 |
| Nucleoside diphosphate kinase beta isoform | 2.5074408 | 2.7934363 | 1.4334546 | 1.0256996 | 1.1966827 | 1.3663622 |
| p55CDC | 0.851024 | 2.416855 | 0.9142256 | 1.0827082 | 0.9610515 | 0.8647809 |
| Phase-1 RCT-156 | 1.8165472 | 1.4256625 | 1.012525 | 1.1063913 | 1.1800071 | 0.8604296 |
| Ribosomal protein L13A | 2.5302038 | 2.829622 | 1.3317788 | 0.9380477 | 0.9932083 | 1.1952212 |
| Phase-1 RCT-258 | 1.1689676 | 1.5689064 | 0.8246077 | 1.0126122 | 1.0265058 | 0.987626 |
| Insulin-like growth factor I | 0.7248876 | 0.4833642 | 1.4077797 | 1.1036257 | 0.9182893 | 0.762094 |
| Cytochrome P450 3A1 | 0.4605225 | 0.1873848 | 0.7314621 | 0.9375491 | 0.5838507 | 0.971742 |
| {Ribosomal protein 16} | 2.8680484 | 2.9866326 | 1.2261413 | 1.1216782 | 1.0852697 | 1.1133198 |
| Organic cation transporter 3 | 1.6903996 | 1.8326681 | 0.9150084 | 1.0142077 | 0.9462636 | 1.068411 |
| Calpain 2 | 1.1883557 | 1.5412446 | 0.890797 | 0.7869698 | 0.9755877 | 0.9363812 |
| Phase-1 RCT-102 | 0.3625044 | 0.2818515 | 1.0833154 | 0.9482374 | 0.6887333 | 0.678258 |
| Equilbrative nitrobenzylthioinosine-sensitive | 0.7287294 | 0.5820092 | 0.921303 | 1.0470701 | 0.9490277 | 1.0415548 |
| nucleoside transporter | | | | | | |
| Phase-1 RCT-227 | 0.8240477 | 0.5551574 | 1.0955145 | 0.9453292 | 0.97183 | 0.8116638 |
| N-hydroxy-2-acetylaminofluorene sulfotransferase (ST1C1) | 0.7662242 | 0.5146129 | 1.0394673 | 1.1583676 | 1.012859 | 1.0534015 |
| Cathepsin S | 1.4216883 | 1.9187645 | 1.0114268 | 1.0867896 | 1.4757975 | 0.9363895 |
| Apolipoprotein CIII | 0.506324 | 0.4101273 | 0.9812037 | 0.8762076 | 0.7562324 | 0.9743487 |
| Cytochrome P450 2A3 | 0.5494803 | 0.4875849 | 0.9457327 | 1.0527964 | 0.7062473 | 0.9731413 |
| Cytochrome P450 2C23 | 1.0780877 | 0.9086638 | 1.4526384 | 0.9391119 | 0.684924 | 0.8498911 |
| Apolipoprotein C1 | 1.2417547 | 0.9166297 | 1.0040104 | 0.8870569 | 0.9035971 | 1.1416918 |
| Betaine homocysteine methyltransferase (BHMT) | 0.6336106 | 0.3860058 | 0.8964349 | 0.7675554 | 1.357828 | 1.0972499 |
| Paraoxonase 1 | 1.0744874 | 0.6814154 | 1.3704696 | 1.3983389 | 0.996643 | 0.6961164 |
| Phase-1 RCT-207 | 1.2155129 | 1.7862506 | 0.6879979 | 1.2464557 | 0.9126077 | 1.0488476 |
| Cathepsin B | 2.3655205 | 2.6556904 | 1.4265133 | 0.9782832 | 1.1089082 | 0.9964173 |
| Phase-1 RCT-144 | 1.3288761 | 1.8062999 | 0.7942173 | 0.855005 | 0.8572915 | 0.8868567 |
| Alpha-1 microglobulin/bikunin precursor (Ambp) | 1.2360779 | 0.727944 | 1.3424433 | 1.1163338 | 1.0670485 | 0.8851687 |
| Cyclin D1 | 1.241705 | 3.0850465 | 1 | 1.0217469 | 1.2867069 | 0.6341727 |
| Presenilin-1 | 0.7061009 | 0.3574869 | 1.0326602 | 0.8008647 | 0.7379825 | 0.6717431 |
| Protein tyrosine phosphatase, receptor type, D | 0.9046126 | 0.634675 | 1.4618047 | 1.6166271 | 1.2708141 | 0.8855172 |
| Multidrug resistant protein-1 | 1.4042009 | 3.4925525 | 1.2675256 | 1.2250693 | 1.1687099 | 0.8691108 |
| Cathepsin L, sequence 2 | 2.4290807 | 2.6707878 | 1.2645764 | 1.2382425 | 1.2356796 | 0.9940368 |
| JNK1 stress activated protein kinase | 0.5928878 | 0.535907 | 0.7989695 | 0.8314123 | 1 | 0.9673644 |
| Phase-1 RCT-43 | 1.5296292 | 1.24384 | 1.072839 | 1.0529513 | 1.1685102 | 0.8653286 |
| Tissue plasminogen activator | 1.6263255 | 1.6341877 | 1.0224247 | 0.9295122 | 0.8748591 | 1.0170196 |
| Ubiquitin conjugating enzyme (RAD 6 | 0.9852406 | 1.1941255 | 0.8046921 | 1.078883 | 0.989395 | 1.0942103 |
| homologue) | | | | | | |
| Phase-1 RCT-173 | 1.2371801 | 1.4621053 | 0.933365 | 0.8453962 | 0.9161639 | 1.147203 |
| Ornithine decarboxylase | 1.1118157 | 2.7286115 | 1.2385048 | 1.2645603 | 1.0781218 | 1.0690637 |
| Zinc finger protein | 0.8843341 | 1.5186821 | 0.7472866 | 0.9014929 | 1.0574871 | 1.1518028 |
| CDK108 | 0.7226736 | 0.7112539 | 0.8987659 | 0.8795282 | 0.9662641 | 0.9768586 |
| Phase-1 RCT-117 | 0.6187307 | 0.6831984 | 0.6773781 | 0.6856109 | 0.9430318 | 1.0886003 |
| {Phase-1 RCT 98} | 0.6690359 | 0.5893254 | 0.7742356 | 0.7826729 | 0.9003356 | 1.0104785 |
| Aquaporin-3 (AQP3) | 0.7060642 | 0.621568 | 0.7905171 | 0.8615832 | 0.8388196 | 0.9116323 |
| Cholesterol esterase | 0.8762791 | 0.5370217 | 1.259425 | 1.3838359 | 0.7520491 | 0.6667786 |
| Ribosomal protein S17 | 2.7267776 | 3.1437125 | 1.1027694 | 0.957657 | 0.9846721 | 1.337588 |
| Poly(ADP-ribose) polymerase | 1.0495669 | 1.6059526 | 1.1377742 | 1.0300105 | 1.1434295 | 0.9962289 |
| Annexin V | 1.1952262 | 1.5578784 | 0.8332222 | 0.8862456 | 0.8842881 | 0.8909894 |
| Phase-1 RCT-33 | 1.1844825 | 1.1145512 | 1.2683542 | 1.2418574 | 1.5663332 | 1.0947392 |
| Phase-1 RCT-61 | 1.0256864 | 0.9557548 | 0.872462 | 0.8422263 | 0.9308941 | 1.0365213 |
| Retinol dehydrogenase type III | 1.1780987 | 0.8190055 | 1.6864208 | 1.1323159 | 0.9542885 | 0.8377162 |
| ATPase inhibitor (rat mitochondrial IF1 protein) | 0.8373012 | 0.6273171 | 1.0888771 | 0.9271033 | 0.8692225 | 1.0659472 |
| Thymidylate synthase | 0.824193 | 1.5886664 | 0.8084859 | 0.8131532 | 0.8942529 | 0.9801306 |
| Interleukin-18 | 0.4462648 | 0.7504155 | 0.7430379 | 0.6848736 | 0.893248 | 1.0632968 |
| Lecithin: cholesterol acyltransferase | 1.2059226 | 0.9650487 | 1.4994947 | 1.311094 | 1.0426079 | 0.8403888 |
| Contrapsin-like protease inhibitor (CPi-21) | 0.8055622 | 0.5646713 | 1.4457471 | 1.3165109 | 1.1340977 | 0.6018067 |
| Proliferating cell nuclear antigen gene | 1.3481904 | 2.810761 | 0.9530206 | 0.8903654 | 0.8840921 | 0.8883243 |
| Phase-1 RCT-230 | 0.8218711 | 0.9556361 | 0.717357 | 0.7309389 | 0.8593866 | 1.1908368 |
| Cytochrome P450 2D18 | 0.9578324 | 0.6640616 | 1.2730256 | 1.2386988 | 1.0121467 | 0.8791995 |
| Phase-1 RCT-48 | 0.9404671 | 0.7023994 | 0.7884075 | 0.8956642 | 0.7439678 | 0.9328737 |
| Phase-1 RCT-292 | 0.8700044 | 0.837733 | 0.7441289 | 0.8901258 | 0.8980454 | 1.0267116 |
| Arginosuccinate synthetase 1 | 0.9450652 | 0.6325415 | 0.9126498 | 0.7825973 | 1.1492939 | 0.9895145 |
| C-reactive protein | 1.0970002 | 0.628374 | 1.4538903 | 1.1412189 | 0.9620909 | 0.8478425 |

| | CAD | CAD | CAD | CAD | CAD | CAR | CAR |
|---|---|---|---|---|---|---|---|
| Compound-Dose | 1 | 1 | 2 | 2 | 2 | 16 | 16 |
| Animal Number | 628 | 629 | 6374 | 638 | 639 | 1857 | 1858 |
| Liver Toxicity Necrosis Classification | no | no | no | no | no | no | no |
| Gene Name | | | | | | | |
| Osteoactivin | 1.0665325 | 1.172157 | 1.7863933 | 1.1931956 | 1.2394882 | 1.1410328 | 1.1045834 |
| Stathmin | 0.8924686 | 1.1206372 | 1.5990474 | 1.1654342 | 0.9641957 | 0.9634252 | 0.9573752 |
| Calpactin I heavy chain | 1.0345411 | 1.081489 | 1.3812785 | 1.1311082 | 1.1323539 | 1.314164 | 1.3332907 |
| Phase-1 RCT-179 | 0.9301364 | 1.148666 | 1.2391739 | 1.0653725 | 0.9198718 | 1.1506094 | 1.0745231 |
| Phase-1 RCT-107 | 0.8175727 | 1.0465444 | 0.9548954 | 0.8368409 | 0.8397681 | 0.8367502 | 1.3881271 |
| Heme oxygenase | 4.4283924 | 1.9643402 | 3.7300348 | 5.5524483 | 2.7775517 | 1.0551637 | 0.9415708 |
| Collagen type II | 0.7092298 | 1.3465931 | 1.2968084 | 0.8572667 | 0.8372812 | 1.3327724 | 0.9586821 |
| Phase-1 RCT-81 | 1.1500531 | 1.1848251 | 0.9794027 | 1.1732895 | 1.0401231 | 1.5941306 | 0.9886299 |
| Glycine methyltransferase | 0.7891117 | 1.5491124 | 1.1693579 | 0.8255915 | 0.6663612 | 0.3698674 | 1.3155429 |
| Cystatin C | 0.8507324 | 0.7998715 | 1.0406905 | 1.1595317 | 0.9524197 | 0.9637139 | 0.8434849 |
| Melanoma-associated antigen ME491 | 1.3276935 | 1.0112805 | 1.225988 | 0.9691445 | 1.2237853 | 1.0808668 | 1.1949859 |
| Phase-1 RCT-146 | 1.0154579 | 1.0528523 | 1.2360928 | 1.0033671 | 0.99033 | 1.1277527 | 1.0746533 |
| Gamma-actin, cytoplasmic | 0.8753353 | 0.7878638 | 0.8837703 | 1.2578299 | 1.1429021 | 1.0803655 | 0.5190387 |
| Adenine nucleotide translocator 1 | 0.6193163 | 0.6674933 | 0.6030012 | 0.5993428 | 0.6427052 | 0.735699 | 0.6964728 |
| Thymosin beta-10 | 1.1918932 | 1.2875615 | 1.3832144 | 0.872734 | 0.7290412 | 1.095068 | 0.8884684 |
| Insulin-like growth factor I, exon 6 | 0.9267264 | 1.2463405 | 0.9409041 | 1.0994027 | 1.0724165 | 0.7080874 | 0.9634857 |
| Phase-1 RCT-78 | 1.1898044 | 1.1710012 | 1.0400124 | 1.1653006 | 0.9329683 | 1.0697051 | 0.945805 |
| Alpha-2-microglobulin | 0.2089558 | 0.4426349 | 0.1687039 | 0.2409789 | 0.2747192 | 0.2093138 | 0.5652893 |
| High affinity IgE receptor gamma chain | 1.2941557 | 1.4825243 | 1.6897525 | 1.502082 | 1.3803049 | 0.867802 | 0.8369 |
| (FcERIgamma) | | | | | | | |
| 60S ribosomal protein L6 | 1.3426235 | 1.3117093 | 1.1569418 | 1.0598022 | 0.8972784 | 0.8809056 | 0.7019345 |
| IgE binding protein | 1.7770077 | 1.6814594 | 2.5282478 | 1.9515463 | 1.4337697 | 1.0296745 | 1.0513932 |
| Phase-1 RCT-149 | 1.4890168 | 1.464944 | 1.3881437 | 1.9877689 | 1.9688145 | 0.9851762 | 0.9285048 |
| Cytochrome P450 2C11 | 0.6028097 | 0.9477837 | 0.4205116 | 0.9673994 | 1.1038461 | 0.9508049 | 0.891623 |
| Uncoupling protein 2 | 0.9717299 | 1.117478 | 1.2188777 | 2.257946 | 0.9121676 | 1.4547507 | 1.1121657 |
| Cofilin | 0.8923175 | 0.8437548 | 1.0488541 | 0.9605853 | 0.9933179 | 0.8451205 | 0.8429514 |
| Alpha-tubulin | 1.1923523 | 1.0086633 | 0.6858735 | 0.6690682 | 0.6693219 | 1.1977386 | 0.8008812 |
| Stearyl-CoA desaturase, liver | 0.6796194 | 0.473863 | 0.2561155 | 0.2089647 | 0.1019947 | 0.4603842 | 0.5290524 |
| Glutathione S-transferase theta-1 | 0.9494705 | 0.9955605 | 0.8407757 | 0.6974619 | 0.7902406 | 1.2562506 | 1.3810698 |
| alpha-1,2-fucosyltrans-ferase | 1.819756 | 1.6383902 | 1.378852 | 1.4670113 | 1.0978142 | 0.8463609 | 0.9678506 |
| Phase-1 RCT-138 | 1.0817136 | 1.3532567 | 1.6149446 | 1.33421 | 1.2496605 | 0.7483162 | 0.8762695 |
| Phase-1 RCT-295 | 1.2761326 | 1.3867217 | 1.609847 | 1.5170978 | 1.1603104 | 1.2869893 | 1.095964 |
| Phase-1 RCT-164 | 0.9940795 | 0.9992349 | 0.5770691 | 0.9048551 | 1.0635598 | 0.7692116 | 1.005794 |
| Alpha-2-macroglobulin, sequence 2 | 1.1143765 | 1.5776662 | 1.4587159 | 1.7416406 | 1.271991 | 0.6311336 | 0.5656821 |
| Biliverdin reductase | 0.8462465 | 1.0904614 | 1.1936193 | 1.1367571 | 1.0836538 | 1.4541475 | 1.2521261 |
| Phase-1 RCT-121 | 1.0005095 | 1.1378088 | 0.7761434 | 0.7417699 | 1.0111972 | 1.318387 | 1.0826743 |
| Dynamin-1 (D100) | 0.9464074 | 0.9948134 | 1.0780069 | 0.9045234 | 0.9281922 | 0.8029439 | 1.1082141 |
| Phase-1 RCT-192 | 0.9806716 | 1.190821 | 1.3025424 | 1.2055568 | 1.2325276 | 0.9189155 | 0.8116658 |
| Fatty acid synthase | 1.6123703 | 1.0368274 | 0.6735802 | 0.7511801 | 0.9439027 | 0.6489452 | 0.4697794 |
| Phase-1 RCT-211 | 1.069343 | 1.3357502 | 1.2159463 | 0.9760068 | 0.8419901 | 1 | 1.241617 |
| Ribosomal protein S9 | 0.9431822 | 0.9310616 | 0.9542063 | 1.0317105 | 1.055616 | 0.9489672 | 0.8111987 |
| NADP-dependent isocitrate dehydrogenase, | 1.3226229 | 1.2645937 | 1.3435832 | 1.3449228 | 1.332339 | 0.5872359 | 0.7139651 |
| cytosolic | | | | | | | |
| Phase-1 RCT-57 | 0.6870161 | 0.9374613 | 0.6754913 | 0.6481478 | 0.9198718 | 1.2267358 | 1.3001399 |
| Phase-1 RCT-36 | 0.9873989 | 1.0683182 | 1.1951753 | 0.8820661 | 0.893429 | 0.8796822 | 0.9222881 |
| Apolipoprotein All | 1.914279 | 2.8755283 | 2.7566767 | 1.6149586 | 0.6085442 | 0.243958 | 0.5343935 |
| Phase-1 RCT-68 | 1.1438953 | 1.1476239 | 1.2638475 | 1.2146407 | 1.1124699 | 1.3149091 | 1.1426486 |
| Multidrug resistant protein-2 | 0.8735251 | 0.7224113 | 1.0488412 | 0.9148727 | 1.0541233 | 2.6751242 | 3.2041466 |
| Phase-1 RCT-39 | 1.0485926 | 0.9116205 | 1.0535443 | 1.0516324 | 1.0853375 | 1.3292146 | 1.3084326 |
| NIPK | 0.7622229 | 0.9013284 | 1.0108167 | 0.8794946 | 0.9776922 | 0.9597599 | 1.0836531 |
| F1-ATPase beta subunit | 1.1004356 | 0.8759993 | 0.7737029 | 1.0615368 | 0.9447535 | 0.727937 | 0.8668815 |
| Elongation factor-1 alpha | 1.5562835 | 1.0978409 | 1.1894644 | 1.3560357 | 1.4248784 | 0.9310616 | 0.8631834 |
| Phase-1 RCT-24 | 0.6409109 | 0.9921196 | 1.2730005 | 0.8852999 | 0.9434848 | 1.1576204 | 0.989994 |
| Beta-tubulin, class I | 0.4336131 | 0.604121 | 1.0210123 | 0.6736149 | 0.9237004 | 1.1049175 | 0.9026383 |
| 14-3-3 zeta | 0.7570067 | 0.8971722 | 0.8197048 | 0.8324761 | 0.9610177 | 1.8772742 | 1.6516932 |
| Alpha-prothymosin | 0.4344814 | 0.5852366 | 0.8201226 | 0.6668491 | 0.7237673 | 0.7955514 | 0.7756829 |
| Phase-1 RCT-58 | 0.7332363 | 1.0820199 | 0.5836889 | 0.6520307 | 0.5677207 | 1.1348852 | 1.1388313 |
| Phase-1 RCT-290 | 0.7725083 | 0.9245088 | 0.634636 | 0.8556271 | 0.5994872 | 0.4366021 | 0.8605556 |
| Transitional endoplasmic reticulum ATPase | 0.8493851 | 0.844127 | 0.7615376 | 0.795564 | 0.8799557 | 0.9157009 | 0.8976296 |
| Beta-actin | 0.5598569 | 0.7980942 | 0.7334646 | 0.9184553 | 1.072198 | 1.8051938 | 1.2402866 |
| Thioredoxin-1 (Trx1) | 1.1017902 | 1.0504053 | 1.2511195 | 1.1940999 | 0.8589835 | 0.7783018 | 0.7764201 |
| Phase-1 RCT-174 | 0.8282465 | 0.8686569 | 0.912202 | 0.762703 | 0.8060606 | 0.9501311 | 1.2181208 |
| Beta-actin, sequence 2 | 2.1499083 | 2.8420577 | 1.9619273 | 1.7954544 | 1.6174324 | 1.1473204 | 0.8185862 |
| Phase-1 RCT-109 | 1.5853276 | 1.9990431 | 1.7071104 | 1.2716018 | 1.0265929 | 0.9317502 | 0.9376971 |
| Phase-1 RCT-154 | 0.9735604 | 0.6455876 | 1.0649309 | 0.9623003 | 1.0062271 | 1.2482911 | 1.2396216 |
| Hypoxanthine-guanine | 0.7480323 | 1.0307884 | 1.042244 | 0.7685344 | 0.7994807 | 0.7278966 | 0.699069 |
| phosphoribosyltransferase | | | | | | | |
| Phase-1 RCT-213 | 1.0399321 | 1.1467838 | 1.0455005 | 0.9837884 | 1.3868836 | 1.0761336 | 0.8982956 |
| Phase-1 RCT-260 | 0.9482416 | 0.9374355 | 0.8586294 | 1.0923365 | 0.8658982 | 1.1921955 | 1.4146999 |
| Phase-1 RCT-12 | 0.6370665 | 0.8158476 | 0.8981123 | 0.7687932 | 1.0772015 | 1.3627151 | 1.1982106 |
| Phase-1 RCT-293 | 1.1559908 | 1.3943821 | 1.5456153 | 1.3557298 | 1.4896467 | 1.0327646 | 0.9481686 |
| Ribosomal protein S8 | 1.5600296 | 1.6450206 | 1.6552973 | 1.901067 | 1.3777891 | 0.7803683 | 0.7400243 |
| Pyruvate kinase, muscle | 0.9030008 | 1.1117191 | 1.319308 | 1.1423928 | 1.1922361 | 1.2966167 | 1.048933 |
| Nucleoside diphosphate kinase beta isoform | 1.5598345 | 1.3298112 | 1.3820508 | 1.3680536 | 1.3470285 | 0.915318 | 0.9119648 |
| p55CDC | 1.1362691 | 0.9416201 | 1.2032914 | 0.8737148 | 0.9082173 | 1.5090599 | 1.2914269 |
| Phase-1 RCT-156 | 0.9854597 | 1.0472499 | 0.9074011 | 0.9969624 | 1.0804905 | 1.1207283 | 0.8971817 |
| Ribosomal protein L13A | 1.197738 | 1.3888159 | 1.8730823 | 1.0759896 | 1.2042919 | 1.0160464 | 0.8730946 |
| Phase-1 RCT-258 | 1.1241978 | 1.1355168 | 0.8524649 | 0.8242143 | 0.8773513 | 0.9443353 | 0.9046371 |
| Insulin-like growth factor I | 1.2053192 | 1.0882589 | 0.9116887 | 1.3351289 | 1.0724293 | 0.5720252 | 0.7364326 |
| Cytochrome P450 3A1 | 1.7691336 | 1.0047569 | 0.8517258 | 0.8732747 | 1.1884547 | 0.5063222 | 0.9456735 |
| {Ribosomal protein 16} | 1.227273 | 1.5133053 | 1.6339314 | 1.7955631 | 1.1418878 | 0.7901301 | 0.7459639 |
| Organic cation transporter 3 | 1.3185735 | 1.2200881 | 0.9468162 | 0.8936224 | 0.9383927 | 0.9368556 | 0.7920389 |
| Calpain 2 | 1.156135 | 1.2218287 | 1.2764401 | 1.3132267 | 1.0137584 | 1.2344598 | 1.0288965 |
| Phase-1 RCT-102 | 0.4241245 | 0.4243819 | 0.3396031 | 0.4810491 | 0.4334896 | 0.4859798 | 0.69264 |
| Equilbrative nitrobenzylthioinosine-sensitive | 0.8217949 | 0.8817737 | 0.7091222 | 0.928183 | 0.8012734 | 0.5358217 | 0.9412717 |
| nucleoside transporter | | | | | | | |
| Phase-1 RCT-227 | 0.8550361 | 1.015839 | 0.7826054 | 0.8020855 | 0.7790318 | 0.9495751 | 1.0038046 |
| N-hydroxy-2-acetylaminofluorene sulfotransferase (ST1C1) | 0.8991498 | 0.9297743 | 0.6120065 | 0.8335594 | 0.6462507 | 0.4344387 | 0.9115717 |
| Cathepsin S | 1.7859417 | 1.3383412 | 1.5229292 | 1.6338322 | 1.4315379 | 1.0652981 | 0.93567 |
| Apolipoprotein CIII | 0.8929502 | 0.9491501 | 0.5910866 | 0.7155482 | 0.8300238 | 0.5102383 | 0.8206225 |
| Cytochrome P450 2A3 | 1.0187438 | 1.131459 | 0.6410759 | 0.8925564 | 1.0149252 | 0.7451435 | 0.4940452 |
| Cytochrome P450 2C23 | 1.0235347 | 0.8270444 | 1.1638527 | 1.5414809 | 1.084284 | 0.5889407 | 0.9144161 |
| Apolipoprotein C1 | 0.8929913 | 0.6479754 | 0.782818 | 0.6029861 | 0.7147776 | 0.2718542 | 0.4962293 |
| Betaine homocysteine methyltransferase (BHMT) | 0.5125967 | 1.1553246 | 0.3418913 | 0.6463215 | 0.2206169 | 0.2256003 | 0.7926675 |
| Paraoxonase 1 | 1.1347339 | 0.7494653 | 0.8385076 | 1.5015268 | 0.9968892 | 0.4986134 | 0.6601311 |
| Phase-1 RCT-207 | 0.8707481 | 0.9999999 | 0.6717801 | 0.7377786 | 1.0055585 | 2.829484 | 1.9779414 |
| Cathepsin B | 1.9319571 | 1.7701547 | 1.5647726 | 1.9731442 | 1.7893926 | 0.8155689 | 0.8912109 |
| Phase-1 RCT-144 | 1.0188708 | 0.9428757 | 1.0447061 | 0.9409286 | 1.0075805 | 1.0124658 | 1.0343456 |
| Alpha-1 microglobulin/bikunin precursor (Ambp) | 1.2558982 | 1.1286409 | 0.9866916 | 1.2606028 | 1.0718158 | 0.7678008 | 0.8660349 |
| Cyclin D1 | 0.7017316 | 0.710397 | 0.7399339 | 0.7142174 | 0.7844181 | 0.6702117 | 0.9803798 |
| Presenilin-1 | 0.5738198 | 0.7886719 | 0.7456757 | 1.0641698 | 0.8211669 | 0.611732 | 1.0616295 |
| Protein tyrosine phosphatase, receptor type, D | 1.89695 | 1.5084424 | 1.3458662 | 2.1130354 | 1.7318372 | 0.7574692 | 0.8938432 |
| Multidrug resistant protein-1 | 0.6721649 | 0.7169963 | 1.1628461 | 0.8237516 | 1.4737546 | 3.3900235 | 2.570621 |
| Cathepsin L, sequence 2 | 1.3227149 | 1.1131485 | 1.7454609 | 2.002509 | 1.6037253 | 1.2681712 | 1.2636157 |
| JNK1 stress activated protein kinase | 1.2810981 | 1.0812016 | 0.8280049 | 1.0060792 | 1.0062686 | 0.6053468 | 0.7656406 |
| Phase-1 RCT-43 | 0.7531917 | 0.8893328 | 0.7254726 | 0.921966 | 0.9486871 | 1.1680924 | 1.0411214 |
| Tissue plasminogen activator | 0.9577281 | 0.9877754 | 1.0392473 | 0.9957702 | 1 | 0.8979803 | 0.8767256 |
| Ubiquitin conjugating enzyme (RAD 6 | 0.9368976 | 0.9410999 | 0.8059041 | 0.812269 | 0.8803934 | 1.1525109 | 1.0265478 |
| homologue) | | | | | | | |
| Phase-1 RCT-173 | 0.9845681 | 1.0495013 | 0.620447 | 0.5858766 | 0.8678386 | 0.8971723 | 1.0118228 |
| Ornithine decarboxylase | 0.6182745 | 0.8202696 | 1.02686 | 0.8163866 | 0.9970223 | 2.0073037 | 1.5556148 |
| Zinc finger protein | 0.9621174 | 1.0438926 | 0.9575198 | 0.8295651 | 0.934473 | 1.42712 | 1.173207 |
| CDK108 | 0.9738033 | 0.7280162 | 0.9559073 | 0.8496109 | 0.9569575 | 1.1121416 | 1.1969793 |
| Phase-1 RCT-117 | 1.6002544 | 1.3578453 | 1.398219 | 1.3642255 | 0.9797704 | 0.89494 | 0.9389309 |
| {Phase-1 RCT 98} | 0.8248341 | 0.8286338 | 0.8260918 | 0.7252418 | 0.8427538 | 1.059587 | 0.9817427 |
| Aquaporin-3 (AQP3) | 0.9641057 | 1.0068259 | 0.9723379 | 0.9904214 | 0.9432337 | 0.9361457 | 0.9744276 |
| Cholesterol esterase | 0.3808171 | 0.4118466 | 0.3168104 | 0.4605023 | 0.4127513 | 0.7597603 | 0.954551 |
| Ribosomal protein S17 | 1.4335613 | 1.5461757 | 1.5287377 | 1.8742467 | 1.373289 | 0.7041142 | 0.5681906 |
| Poly(ADP-ribose) polymerase | 1.0184307 | 0.8967898 | 1.0198525 | 1.0083522 | 0.9726473 | 1.0639886 | 1.0738014 |
| Annexin V | 1.1425751 | 1.282336 | 1.2745267 | 1.1334479 | 1.110877 | 1.3277035 | 1.0342758 |
| Phase-1 RCT-33 | 1.0288372 | 1.3524175 | 1.8865976 | 0.9478841 | 0.7853419 | 0.7704074 | 0.8615724 |
| Phase-1 RCT-61 | 0.8507804 | 0.8988921 | 0.786226 | 0.8167063 | 0.8533533 | 1.1351812 | 1.1448225 |
| Retinol dehydrogenase type III | 0.901364 | 0.9405818 | 0.9431258 | 1.0871935 | 1.1434933 | 0.55818 | 0.8032016 |
| ATPase inhibitor (rat mitochondrial IF1 protein) | 1.2079859 | 1.1646607 | 1.1230413 | 1.2803812 | 1.0487993 | 0.2823229 | 0.4733359 |
| Thymidylate synthase | 1.1404909 | 0.9548865 | 1.0615398 | 0.7543007 | 1.1198438 | 1.1558344 | 1.1762028 |
| Interleukin-18 | 1.1753737 | 1.0624914 | 1.308324 | 0.5573158 | 1.0013977 | 1.3094553 | 1.0498326 |
| Lecithin: cholesterol acyltransferase | 1.321659 | 1.5550113 | 1.8204037 | 1.4034852 | 1.6003884 | 0.6808284 | 0.8618742 |
| Contrapsin-like protease inhibitor (CPi-21) | 0.5127317 | 0.5320403 | 0.4903969 | 0.6439108 | 0.5303774 | 0.6073504 | 0.8791866 |
| Proliferating cell nuclear antigen gene | 1.0724043 | 0.9362658 | 1.2198149 | 0.8378839 | 0.8969523 | 1.3174077 | 1.150227 |
| Phase-1 RCT-230 | 1.0748166 | 0.8923891 | 0.9961011 | 1.2207761 | 1.0037527 | 1.3190461 | 1.1389757 |
| Cytochrome P450 2D18 | 0.4880095 | 0.8907357 | 1.1645223 | 0.9351507 | 1.279084 | 0.7288252 | 1.22754 |
| Phase-1 RCT-48 | 0.5973459 | 0.8367961 | 0.6806438 | 0.8390214 | 0.8838291 | 0.6178008 | 0.6613509 |
| Phase-1 RCT-292 | 0.9498037 | 1.0773171 | 1.1744982 | 1.1335309 | 1.1038461 | 0.821209 | 0.9408312 |
| Arginosuccinate synthetase 1 | 0.5978172 | 0.7194794 | 0.5970714 | 0.6855367 | 0.4319624 | 0.4093651 | 0.6670509 |
| C-reactive protein | 1.9115757 | 1.5307462 | 1.3286289 | 2.2084036 | 2.2745624 | 0.9814883 | 1.1557539 |

| | CAR | CCL4 | CCL4 | CCL4 | CCL4 | CCL4 |
|---|---|---|---|---|---|---|
| Compound-Dose | 16 | 250 | 250 | 250 | 1000 | 1000 |
| Animal Number | 1859 | 2047 | 2048 | 2049 | 2057 | 2058 |
| Liver Toxicity Necrosis Classification | no | yes | yes | yes | yes | yes |
| Gene Name | | | | | | |
| Osteoactivin | 1.0791085 | 2.0058315 | 1.9926043 | 1.8306382 | 7.446975 | 5.5573606 |
| Stathmin | 1.0127932 | 1.4218938 | 1.7862334 | 1.3602635 | 1.7461764 | 2.222967 |
| Calpactin I heavy chain | 1.2505677 | 1.2960685 | 1.3677496 | 1.2208787 | 2.5260177 | 2.8906698 |
| Phase-1 RCT-179 | 1.1404939 | 0.9691183 | 1.2330016 | 0.9291534 | 1.2264303 | 1.5598841 |
| Phase-1 RCT-107 | 1.0904388 | 0.6383804 | 0.5097365 | 0.8211671 | 0.4635037 | 0.3759924 |
| Heme oxygenase | 0.9148829 | 1.2192881 | 1.2521654 | 1.2716236 | 2.6257865 | 2.5839436 |
| Collagen type II | 1.0574185 | 1.7398632 | 1.8588164 | 2.2144415 | 2.5078828 | 5.842111 |
| Phase-1 RCT-81 | 0.9945181 | 0.9879189 | 0.9824467 | 1.0792885 | 0.5656784 | 0.5970601 |
| Glycine methyltransferase | 1.214059 | 0.498161 | 0.3624067 | 0.7805239 | 0.2308108 | 0.1651169 |
| Cystatin C | 0.8354943 | 1.1297824 | 1.1546545 | 1.2230195 | 1.4451475 | 1.7024032 |
| Melanoma-associated antigen ME491 | 1.0900705 | 1.4723765 | 1.2686578 | 1.2509673 | 2.8335657 | 2.332399 |
| Phase-1 RCT-146 | 1.0489885 | 1.0928171 | 1.280334 | 1.1679989 | 1.6007707 | 2.056257 |
| Gamma-actin, cytoplasmic | 0.8082217 | 1.2430302 | 1.0796582 | 0.9244675 | 2.1523516 | 1.6232893 |
| Adenine nucleotide translocator 1 | 0.7973023 | 0.93568 | 1.0603166 | 1.132793 | 1.139911 | 1.3874444 |
| Thymosin beta-10 | 1.1026716 | 1.0451312 | 0.9885427 | 0.9603996 | 1.7524388 | 1.7668253 |
| Insulin-like growth factor I, exon 6 | 0.7843484 | 0.7107506 | 0.8337696 | 0.811426 | 0.4083999 | 0.4208347 |
| Phase-1 RCT-78 | 0.9296351 | 0.9067008 | 0.8544339 | 0.8181267 | 0.6618262 | 0.6763603 |
| Alpha-2-microglobulin | 0.5650264 | 0.5423474 | 0.4783321 | 0.4038144 | 0.0455277 | 0.0387278 |
| High affinity IgE receptor gamma chain | 0.910727 | 1.1453834 | 1.254547 | 1.1729321 | 1.4901124 | 1.7922894 |
| (FcERIgamma) | | | | | | |
| 60S ribosomal protein L6 | 0.8335336 | 1.1025578 | 1.3074186 | 1.038103 | 1.6391575 | 1.9380125 |
| IgE binding protein | 0.9734221 | 1.1097196 | 1.1591631 | 1.1494908 | 2.9945643 | 2.700304 |
| Phase-1 RCT-149 | 0.9893558 | 0.8937365 | 0.8344536 | 0.8894683 | 0.7632833 | 0.7374014 |
| Cytochrome P450 2C11 | 0.7622576 | 0.7230483 | 0.6026726 | 0.6021777 | 0.1812755 | 0.4475355 |
| Uncoupling protein 2 | 1.3100665 | 1.11779 | 1.0441632 | 1.1008842 | 2.0523226 | 2.3215685 |
| Cofilin | 0.898046 | 1.0684772 | 1.2092881 | 1.1345481 | 1.2444086 | 1.466486 |
| Alpha-tubulin | 1.0400246 | 1.6633117 | 1.3153723 | 1.3282275 | 2.284918 | 2.1531665 |
| Stearyl-CoA desaturase, liver | 0.5887289 | 1.0701383 | 0.229748 | 1.797065 | 0.1199332 | 0.1398464 |
| Glutathione S-transferase theta-1 | 1.8597336 | 1.6680232 | 1.5281421 | 1.2046309 | 1.3708122 | 1.20407 |
| alpha-1,2-fucosyltrans-ferase | 0.927978 | 0.8444331 | 0.7947701 | 0.805593 | 0.8266462 | 0.9107999 |
| Phase-1 RCT-138 | 0.8806634 | 1.0421757 | 1.1127105 | 1.1065526 | 1.6298089 | 1.8006104 |
| Phase-1 RCT-295 | 1.24673 | 1.4656634 | 1.538085 | 1.2423893 | 2.4677396 | 2.5078368 |
| Phase-1 RCT-164 | 0.9086702 | 0.9232338 | 0.7619515 | 0.8737822 | 0.4343247 | 0.4640241 |
| Alpha-2-macroglobulin, sequence 2 | 0.5982199 | 1.1005363 | 1.1233308 | 1.0272374 | 1.2770689 | 1.4651927 |
| Biliverdin reductase | 1.2213544 | 1.1003774 | 1.316425 | 1.2566149 | 1.7628571 | 1.6647373 |
| Phase-1 RCT-121 | 1.1381892 | 1.0425304 | 1.0895827 | 1.0205204 | 1.4909078 | 1.8977509 |
| Dynamin-1 (D100) | 0.8801926 | 0.9682944 | 1.041187 | 0.9091677 | 0.6496497 | 0.7830657 |
| Phase-1 RCT-192 | 0.9852801 | 1.3794179 | 1.4602945 | 1.1725944 | 2.8850422 | 2.5080056 |
| Fatty acid synthase | 0.5170938 | 0.9471612 | 0.2838945 | 0.9246498 | 0.4349518 | 0.3518697 |
| Phase-1 RCT-211 | 1.1506641 | 1.3178092 | 1.4913837 | 1.065474 | 1.7762886 | 1.7880337 |
| Ribosomal protein S9 | 1.0082144 | 1.0928938 | 1.3019788 | 1.143338 | 1.5533175 | 1.7631791 |
| NADP-dependent isocitrate dehydrogenase, | 0.6941201 | 0.9715809 | 1.246839 | 0.9187357 | 0.9495956 | 1.1231108 |
| cytosolic | | | | | | |
| Phase-1 RCT-57 | 1.2082529 | 0.8555652 | 0.7164716 | 0.8886977 | 0.5672821 | 0.487289 |
| Phase-1 RCT-36 | 0.9113312 | 0.9370076 | 0.8312611 | 0.8353079 | 0.7829314 | 0.84219 |
| Apolipoprotein All | 0.4767206 | 0.7002696 | 0.3586386 | 0.4930623 | 0.2236521 | 0.2851685 |
| Phase-1 RCT-68 | 1.1229688 | 1.1710354 | 1.3016754 | 1.0428538 | 1.3228114 | 1.3562137 |
| Multidrug resistant protein-2 | 2.5671992 | 1.3324882 | 1.3615278 | 1.4796067 | 2.6763613 | 2.0682933 |
| Phase-1 RCT-39 | 1.2386647 | 1.1758094 | 1.1633307 | 1.1703159 | 1.6035161 | 1.4508458 |
| NIPK | 0.8674085 | 0.8205677 | 0.8217452 | 0.8249558 | 0.610263 | 0.645766 |
| F1-ATPase beta subunit | 0.9297035 | 1.3603851 | 1.4699074 | 1.6168358 | 1.6181108 | 1.5368966 |
| Elongation factor-1 alpha | 1.0672668 | 1.2581617 | 1.1725564 | 1.1921073 | 1.6285516 | 1.6479585 |
| Phase-1 RCT-24 | 1.0271378 | 1.9807458 | 1.4645808 | 1.4525738 | 2.6398728 | 2.2371125 |
| Beta-tubulin, class I | 0.9322754 | 2.03773 | 1.5125707 | 1.7723317 | 2.8189354 | 2.4194798 |
| 14-3-3 zeta | 1.6336448 | 1.2152431 | 1.4221846 | 1.0976837 | 1.861678 | 1.8765299 |
| Alpha-prothymosin | 0.8434385 | 1.1708087 | 1.4159213 | 1.0929669 | 1.2786278 | 1.654276 |
| Phase-1 RCT-58 | 1.2213385 | 1.4791176 | 1.5625839 | 1.1261069 | 1.2397317 | 1.2317313 |
| Phase-1 RCT-290 | 0.5210409 | 0.6957201 | 0.4584549 | 1.0227278 | 0.4113167 | 0.3697359 |
| Transitional endoplasmic reticulum ATPase | 0.9132015 | 0.995198 | 1.0017833 | 1.034594 | 1.0158985 | 0.9598438 |
| Beta-actin | 1.5750666 | 1.4084526 | 1.1297388 | 1.183385 | 2.0807853 | 2.6451986 |
| Thioredoxin-1 (Trx1) | 0.9065911 | 1.0490075 | 1.1167916 | 0.9045495 | 1.3035374 | 1.4416311 |
| Phase-1 RCT-174 | 0.996861 | 0.956472 | 0.9130586 | 1.1261696 | 0.6575568 | 0.6851393 |
| Beta-actin, sequence 2 | 1.0506712 | 1.328087 | 1.169625 | 1.0848064 | 1.6233562 | 1.6378309 |
| Phase-1 RCT-109 | 0.9628838 | 1.1022388 | 1.0939122 | 1.0216124 | 1.4954778 | 1.4311703 |
| Phase-1 RCT-154 | 1.1304674 | 1.0653132 | 1.3830305 | 0.956562 | 1.3780336 | 1.6100153 |
| Hypoxanthine-guanine | 0.7886922 | 1.0057204 | 1.0234536 | 1.0276684 | 1.4189752 | 1.2745352 |
| phosphoribosyltransferase | | | | | | |
| Phase-1 RCT-213 | 0.9799548 | 1.03653 | 1.034798 | 1.0038812 | 1.2449272 | 1.1308129 |
| Phase-1 RCT-260 | 1.0786743 | 0.9314588 | 0.9184816 | 0.9462087 | 0.9011378 | 0.782112 |
| Phase-1 RCT-12 | 1.1830252 | 1.5257219 | 1.3157964 | 1.4784877 | 1.9061862 | 1.7596933 |
| Phase-1 RCT-293 | 1.0649359 | 1.0060487 | 1.1000072 | 0.999013 | 1.4766525 | 2.2657673 |
| Ribosomal protein S8 | 0.8297668 | 1.2437532 | 1.3446913 | 1.0737239 | 1.7069017 | 2.198003 |
| Pyruvate kinase, muscle | 1.091483 | 1.1180258 | 0.8315749 | 1.0811096 | 1.6411086 | 1.6721833 |
| Nucleoside diphosphate kinase beta isoform | 1.2081472 | 1.3119549 | 1.4543111 | 1.2710446 | 2.3024597 | 2.0810084 |
| p55CDC | 1.1103399 | 1.2920731 | 0.9040536 | 1.2825875 | 1.28513 | 1.2843177 |
| Phase-1 RCT-156 | 1.1516382 | 0.9995588 | 0.9827669 | 1.0037644 | 1.0984148 | 0.9904751 |
| Ribosomal protein L13A | 1.0321352 | 1.0548769 | 1.0254123 | 0.9730259 | 1.5813111 | 1.5214164 |
| Phase-1 RCT-258 | 1.0051786 | 1.0333644 | 1.0526334 | 0.9677312 | 1.0815375 | 1.0870847 |
| Insulin-like growth factor I | 0.5989702 | 0.8062836 | 0.852684 | 0.746143 | 0.4339331 | 0.4749387 |
| Cytochrome P450 3A1 | 1.0653609 | 1.0453829 | 1.3726258 | 0.6011971 | 0.698145 | 1.0172484 |
| {Ribosomal protein 16} | 0.8311378 | 1.1377889 | 1.2077099 | 1.0808362 | 1.5614978 | 1.6835623 |
| Organic cation transporter 3 | 0.8931727 | 1.1210359 | 1.2935792 | 1.055209 | 1.5646762 | 1.8374114 |
| Calpain 2 | 1.0828367 | 1.0711572 | 1.0701485 | 1.0341055 | 1.3630621 | 1.4449853 |
| Phase-1 RCT-102 | 0.4403092 | 1.0744388 | 0.7977458 | 0.6492831 | 0.5089333 | 0.5532627 |
| Equilbrative nitrobenzylthioinosine-sensitive | 0.8191195 | 0.9312632 | 0.9449444 | 0.9103911 | 0.5623143 | 0.45571 |
| nucleoside transporter | | | | | | |
| Phase-1 RCT-227 | 1.0346432 | 0.8387797 | 0.9129243 | 0.7456406 | 0.6174007 | 0.6470146 |
| N-hydroxy-2-acetylaminofluorene sulfotransferase (ST1C1) | 0.7448509 | 0.8269094 | 0.8834655 | 0.7622323 | 0.2938168 | 0.3189412 |
| Cathepsin S | 0.998913 | 1.0891922 | 1.1142695 | 1.1235511 | 1.574982 | 2.0671692 |
| Apolipoprotein CIII | 0.6803416 | 0.8295034 | 0.7365158 | 0.9398652 | 0.6701827 | 0.5761476 |
| Cytochrome P450 2A3 | 0.4994638 | 0.9930812 | 0.9081992 | 0.8914011 | 0.7303426 | 0.7709543 |
| Cytochrome P450 2C23 | 0.8707982 | 0.7163075 | 0.6828682 | 0.8536478 | 0.3115271 | 0.4020347 |
| Apolipoprotein C1 | 0.4134154 | 0.6146088 | 0.8989736 | 0.7776749 | 0.265843 | 0.2353298 |
| Betaine homocysteine methyltransferase (BHMT) | 0.3713349 | 0.6481938 | 0.4057396 | 1.0057766 | 0.3718656 | 0.2902318 |
| Paraoxonase 1 | 0.5678107 | 0.8054301 | 0.9786457 | 0.950003 | 0.4079569 | 0.456519 |
| Phase-1 RCT-207 | 1.771263 | 1.0666054 | 1.2031789 | 1.0610529 | 1.4129148 | 1.4194102 |
| Cathepsin B | 0.8324128 | 1.0605543 | 1.0255699 | 1.2677077 | 2.1628554 | 1.9978731 |
| Phase-1 RCT-144 | 0.9233654 | 1.1031113 | 1.1516855 | 1.038018 | 1.3564309 | 1.3619854 |
| Alpha-1 microglobulin/bikunin precursor (Ambp) | 0.9077508 | 0.912204 | 0.9538372 | 1.0835345 | 0.4672214 | 0.4678266 |
| Cyclin D1 | 0.9509335 | 1.3488333 | 1.579639 | 1.4743768 | 2.4036522 | 2.7915921 |
| Presenilin-1 | 0.6864462 | 0.9688051 | 0.7020629 | 1.132715 | 0.3554994 | 0.2634515 |
| Protein tyrosine phosphatase, receptor type, D | 0.8928595 | 0.7922792 | 0.8701066 | 0.8656213 | 0.2919095 | 0.3406438 |
| Multidrug resistant protein-1 | 3.0859964 | 1.3651092 | 1.2567679 | 1.469048 | 2.4378831 | 1.7078879 |
| Cathepsin L, sequence 2 | 1.6109526 | 0.9921255 | 0.9381607 | 1.0150248 | 1.2866553 | 1.2415595 |
| JNK1 stress activated protein kinase | 0.8061491 | 0.8051629 | 0.865966 | 0.9665052 | 0.6890652 | 0.6918693 |
| Phase-1 RCT-43 | 1.0983555 | 0.9652509 | 0.9488797 | 0.9576113 | 1.0312893 | 0.9749386 |
| Tissue plasminogen activator | 0.9667089 | 1.0353717 | 1.0576382 | 1.0456386 | 1.0468285 | 1.206319 |
| Ubiquitin conjugating enzyme (RAD 6 | 1.1984125 | 1.1691686 | 1.3387586 | 1.1723818 | 1.5057485 | 1.8342779 |
| homologue) | | | | | | |
| Phase-1 RCT-173 | 0.9821044 | 1.1899408 | 1.3671306 | 1.293887 | 1.6699445 | 1.737661 |
| Ornithine decarboxylase | 1.7868443 | 1.0923878 | 1.2409372 | 1.1308789 | 1.3873127 | 1.1308532 |
| Zinc finger protein | 1.1812947 | 1.03759 | 1.1270909 | 0.9707479 | 0.9843267 | 1.0974257 |
| CDK108 | 1.0218396 | 1.1768365 | 0.966158 | 0.9107066 | 1.3009584 | 1.2725173 |
| Phase-1 RCT-117 | 1.0350174 | 1.0443425 | 0.7876722 | 1.1318004 | 0.7268967 | 0.6568555 |
| {Phase-1 RCT 98} | 0.9040024 | 0.7854089 | 0.8860593 | 1.0058944 | 0.7996476 | 0.7604342 |
| Aquaporin-3 (AQP3) | 0.9123896 | 0.8421468 | 0.877265 | 0.9126553 | 0.7851493 | 0.759188 |
| Cholesterol esterase | 1.0782436 | 1.004791 | 0.9382269 | 0.8601152 | 0.4412271 | 0.595129 |
| Ribosomal protein S17 | 0.6507357 | 1.2143977 | 1.221496 | 1.1047949 | 1.9363995 | 2.0189354 |
| Poly(ADP-ribose) polymerase | 1.033394 | 1.0727965 | 1.1341121 | 0.9873142 | 1.340558 | 1.3387599 |
| Annexin V | 1.2647789 | 1.2309636 | 1.1099993 | 1.0752969 | 1.5176529 | 1.6017667 |
| Phase-1 RCT-33 | 0.8518751 | 0.855602 | 0.7185448 | 0.7864071 | 0.6406933 | 0.695174 |
| Phase-1 RCT-61 | 1.0415943 | 1.2517315 | 1.2694409 | 1.0651435 | 1.2076063 | 1.1866517 |
| Retinol dehydrogenase type III | 0.8679815 | 0.9412388 | 0.7640453 | 0.9126665 | 0.3566563 | 0.403525 |
| ATPase inhibitor (rat mitochondrial IF1 protein) | 0.4346601 | 0.7327986 | 0.7999811 | 0.5858128 | 0.1523169 | 0.2062801 |
| Thymidylate synthase | 1.1965787 | 1.3186125 | 1.0728673 | 1.0500435 | 1.349954 | 1.3929555 |
| Interleukin-18 | 1.052132 | 1.1009932 | 1.181108 | 1.0451354 | 1.6058179 | 1.6184607 |
| Lecithin: cholesterol acyltransferase | 0.9004336 | 0.8325958 | 0.9654122 | 0.8707223 | 0.6733493 | 0.6884756 |
| Contrapsin-like protease inhibitor (CPi-21) | 0.7750425 | 0.7687844 | 0.7867705 | 0.9287353 | 0.3505022 | 0.3073565 |
| Proliferating cell nuclear antigen gene | 1.0542336 | 1.0520881 | 1.3001784 | 1.0824805 | 1.7270813 | 1.9438897 |
| Phase-1 RCT-230 | 1.192879 | 0.9772837 | 0.9014226 | 1.042058 | 1.4280938 | 1.564372 |
| Cytochrome P450 2D18 | 1.0496057 | 0.7857627 | 0.637921 | 0.9022843 | 0.4843491 | 0.3475778 |
| Phase-1 RCT-48 | 0.6581985 | 0.9522173 | 0.8840928 | 0.8335438 | 0.8342989 | 0.830525 |
| Phase-1 RCT-292 | 0.9529738 | 0.8467591 | 0.8292478 | 0.8989288 | 0.8093618 | 0.8462255 |
| Arginosuccinate synthetase 1 | 0.5820099 | 0.7685763 | 0.5360118 | 0.9265258 | 0.3698375 | 0.3233079 |
| C-reactive protein | 1.2500973 | 0.8058755 | 0.9344842 | 0.8592742 | 0.3003292 | 0.4002322 |

| | CCL4 | CHCL3 | CHCL3 | CHCL3 | CHEX | CHEX | CHEX |
|---|---|---|---|---|---|---|---|
| Compound-Dose | 1000 | 250 | 250 | 500 | 0.5 | 0.5 | 0.5 |
| Animal Number | 2059 | 1627 | 1628 | 2358 | 2247 | 2248 | 2249 |
| Liver Toxicity Necrosis Classification | yes | no | no | no | no | no | no |
| Gene Name | | | | | | | |
| Osteoactivin | 5.130466 | 1.0019327 | 0.9442192 | 1.8934748 | 1.0518706 | 0.9638626 | 0.9808977 |
| Stathmin | 1.5889753 | 0.9431016 | 0.9773857 | 0.8051049 | 1.1721686 | 1.1464889 | 1.0022078 |
| Calpactin I heavy chain | 1.7168921 | 1.3199887 | 1.2730043 | 1.0386509 | 1.1596144 | 1.1497961 | 1.0895469 |
| Phase-1 RCT-179 | 0.9866501 | 0.9120464 | 0.9248939 | 1.1638247 | 1.033609 | 1.0704336 | 1.2966669 |
| Phase-1 RCT-107 | 0.7235594 | 1.0436077 | 1.2750189 | 1.2525789 | 1.2013826 | 0.979107 | 0.8935429 |
| Heme oxygenase | 2.1026013 | 1.0900195 | 0.9201754 | 1.8280262 | 0.9902325 | 1.0394047 | 1.050774 |
| Collagen type II | 1.8792013 | 1.0109729 | 0.988785 | 1.1085094 | 1.6817974 | 0.6587873 | 0.5973096 |
| Phase-1 RCT-81 | 0.8007692 | 0.8153347 | 0.9836091 | 1.0153191 | 1.1661291 | 1.132924 | 1.0843575 |
| Glycine methyltransferase | 0.4690456 | 1.1719779 | 1.4642136 | 1.4589471 | 1.7805102 | 1.0661333 | 1.0144597 |
| Cystatin C | 1.142634 | 1.086228 | 0.8840182 | 1.305763 | 1.0190334 | 1.0073808 | 1.0566652 |
| Melanoma-associated antigen ME491 | 1.8273805 | 1.456671 | 1.2362423 | 1.3471775 | 1.1931217 | 1.4899769 | 1.2056074 |
| Phase-1 RCT-146 | 1.2065194 | 1.0837922 | 1.1216372 | 0.9409012 | 0.9062533 | 0.9263135 | 0.8631803 |
| Gamma-actin, cytoplasmic | 1.0627815 | 0.8789395 | 1.0041891 | 0.6871958 | 0.8361991 | 0.8952676 | 0.8963105 |
| Adenine nucleotide translocator 1 | 1.0846702 | 0.8737416 | 0.9582123 | 0.7813078 | 0.8605819 | 0.9826585 | 1.09639 |
| Thymosin beta-10 | 1.3400304 | 0.9348089 | 0.9633579 | 1.2734748 | 1.0689707 | 1.0296606 | 1.1555454 |
| Insulin-like growth factor I, exon 6 | 0.4131315 | 0.8968087 | 0.8548319 | 0.5789639 | 1.2925777 | 1.1359165 | 1.2867465 |
| Phase-1 RCT-78 | 0.7043539 | 0.8934571 | 1.0297524 | 0.9750135 | 0.9364227 | 1.1501502 | 1.0569673 |
| Alpha-2-microglobulin | 0.0397795 | 0.5649121 | 0.6618636 | 0.2335385 | 1.5461295 | 1.1629432 | 0.9915668 |
| High affinity IgE receptor gamma chain | 1.4894341 | 1.006549 | 0.9437802 | 1.0529372 | 0.8970746 | 0.9213709 | 1.0590955 |
| (FcERIgamma) | | | | | | | |
| 60S ribosomal protein L6 | 1.1257768 | 1.3277925 | 1.050002 | 1.2428069 | 1.0248216 | 0.9901906 | 1.1415035 |
| IgE binding protein | 1.6547767 | 0.9654609 | 0.8789625 | 0.9535868 | 1.0880333 | 0.9971219 | 1.1608648 |
| Phase-1 RCT-149 | 0.8275476 | 0.8573623 | 0.9560632 | 0.8831542 | 1.1105013 | 1.0499421 | 0.8877013 |
| Cytochrome P450 2C11 | 0.1223874 | 0.8732856 | 0.9999309 | 0.0681397 | 0.8999035 | 0.8483307 | 0.8750407 |
| Uncoupling protein 2 | 1.6471984 | 0.8512478 | 0.8524129 | 1.1010869 | 0.8877048 | 0.8585364 | 0.9524964 |
| Cofilin | 1.1823217 | 1.0015084 | 0.9893438 | 0.8575822 | 1.2096004 | 1.1579587 | 1.247941 |
| Alpha-tubulin | 1.0031354 | 1.0154225 | 0.9986309 | 0.7140515 | 1.0094421 | 1.1205623 | 1.2136158 |
| Stearyl-CoA desaturase, liver | 0.2190726 | 0.2915084 | 1.2665225 | 0.0274851 | 1.9898174 | 3.1347382 | 2.0735362 |
| Glutathione S-transferase theta-1 | 2.140176 | 1.0133662 | 1.0501382 | 0.7915426 | 0.9385146 | 0.9755815 | 0.9051133 |
| alpha-1,2-fucosyltrans-ferase | 0.8907068 | 0.8793349 | 0.8504782 | 1.1342447 | 1.1568007 | 1.0579782 | 0.8907793 |
| Phase-1 RCT-138 | 1.4304013 | 1.1034963 | 1.042673 | 1.2854482 | 1.1836574 | 1.1525484 | 1.0789376 |
| Phase-1 RCT-295 | 1.5859789 | 1.1013749 | 1.0214736 | 1.0745094 | 1.115104 | 1.1720223 | 1.2773601 |
| Phase-1 RCT-164 | 0.6984019 | 0.9783812 | 1.0270288 | 0.9265081 | 1.0273719 | 0.9996253 | 0.9599887 |
| Alpha-2-macroglobulin, sequence 2 | 1.3612596 | 1.1521635 | 1.1422917 | 1.3800397 | 0.9396658 | 0.9299302 | 0.9858072 |
| Biliverdin reductase | 1.5416354 | 0.9222022 | 1.137499 | 0.9333647 | 0.8804063 | 0.863592 | 1.0341108 |
| Phase-1 RCT-121 | 1.3770657 | 0.9787075 | 0.9614344 | 1.1636872 | 0.8011881 | 0.7679898 | 0.9735252 |
| Dynamin-1 (D100) | 0.6644468 | 0.9105309 | 0.9834482 | 0.8640322 | 1.1829777 | 1.0817814 | 1.0112854 |
| Phase-1 RCT-192 | 1.4288508 | 0.9475594 | 1.0122488 | 1.4300902 | 0.8955791 | 0.8921563 | 1.0543712 |
| Fatty acid synthase | 0.4458792 | 0.7714209 | 1.3481869 | 0.1610174 | 1.6320819 | 1.6048585 | 0.9614196 |
| Phase-1 RCT-211 | 1.2305915 | 1.1249061 | 1.0400891 | 1.1118251 | 1.3850499 | 1.2005726 | 1.0705174 |
| Ribosomal protein S9 | 1.2107971 | 0.907597 | 0.9195357 | 0.9119539 | 1.0193293 | 0.9285519 | 1.2878051 |
| NADP-dependent isocitrate dehydrogenase, | 0.9924637 | 1.0174515 | 0.958591 | 0.8399722 | 1.1062666 | 1.3685269 | 1.3761585 |
| cytosolic | | | | | | | |
| Phase-1 RCT-57 | 0.7867349 | 0.9800588 | 1.0156409 | 1.1173589 | 0.9422626 | 0.9365945 | 0.8321246 |
| Phase-1 RCT-36 | 0.873698 | 0.9389271 | 1.253699 | 0.9678064 | 0.9348829 | 1.0320553 | 0.8578177 |
| Apolipoprotein All | 0.2509521 | 0.7524521 | 0.7182068 | 0.8223169 | 1.3266569 | 0.8107357 | 0.7316257 |
| Phase-1 RCT-68 | 1.2798647 | 0.9791547 | 1.0247414 | 1.1062497 | 1.0076649 | 1.0164758 | 1.0128443 |
| Multidrug resistant protein-2 | 2.177902 | 1.1354089 | 1.0074447 | 1.121206 | 0.7261847 | 0.8042728 | 0.7663062 |
| Phase-1 RCT-39 | 1.2490991 | 1.0070091 | 0.9854727 | 1.1365848 | 0.8045235 | 0.9028392 | 0.9540967 |
| NIPK | 0.7734674 | 1.181947 | 0.8911056 | 0.7975655 | 0.9819258 | 0.9115132 | 0.9182284 |
| F1-ATPase beta subunit | 1.5691476 | 1.0026221 | 0.9346185 | 0.9999909 | 1.3848963 | 1.3716176 | 1.2767359 |
| Elongation factor-1 alpha | 1.2703656 | 1.1160345 | 0.9045163 | 1.4538318 | 1.3664104 | 1.197687 | 1.179399 |
| Phase-1 RCT-24 | 0.9268476 | 1.0037526 | 0.9719353 | 0.726193 | 0.7910027 | 0.7141856 | 0.803393 |
| Beta-tubulin, class I | 1.2598692 | 1.103874 | 1.0472581 | 0.6566534 | 1.0864856 | 1.1932161 | 1.3254426 |
| 14-3-3 zeta | 1.2699434 | 0.9437727 | 0.9141008 | 0.7747644 | 1.115871 | 1.209033 | 1.3201776 |
| Alpha-prothymosin | 1.1132011 | 0.9631168 | 0.9509075 | 0.8346818 | 1.1375437 | 1.1632429 | 1.3590928 |
| Phase-1 RCT-58 | 1.6420472 | 1.3391933 | 0.89872 | 1.1707473 | 1.1122411 | 1.5042058 | 1.0850564 |
| Phase-1 RCT-290 | 0.7247496 | 0.950097 | 1.5939356 | 0.5880656 | 1.4030374 | 1.3823284 | 1.158624 |
| Transitional endoplasmic reticulum ATPase | 0.920731 | 0.9883732 | 1.0306814 | 0.9265487 | 0.6634756 | 0.6542198 | 0.6117549 |
| Beta-actin | 1.2667081 | 0.8540543 | 0.8016556 | 0.5115773 | 1.2885835 | 1.3662705 | 1.2320995 |
| Thioredoxin-1 (Trx1) | 0.9854693 | 0.9148518 | 0.8513995 | 0.8202065 | 1.0833783 | 0.9342011 | 1.0919899 |
| Phase-1 RCT-174 | 0.8542789 | 0.9654592 | 1.0192283 | 0.6345472 | 1.109999 | 0.9232695 | 0.9923904 |
| Beta-actin, sequence 2 | 1.0991755 | 0.9682863 | 0.901458 | 0.6618419 | 1.3721788 | 1.3387305 | 1.246772 |
| Phase-1 RCT-109 | 1.1656619 | 0.9635379 | 1.0213104 | 1.2865695 | 0.9872579 | 0.919766 | 0.6319838 |
| Phase-1 RCT-154 | 0.9478831 | 1.0839413 | 1.0796214 | 0.9112034 | 0.9526295 | 0.9108883 | 1.072916 |
| Hypoxanthine-guanine | 1.1542877 | 0.9481685 | 1.2644359 | 0.7598425 | 1.2068825 | 1.3194538 | 1.0690366 |
| phosphoribosyltransferase | | | | | | | |
| Phase-1 RCT-213 | 0.9734225 | 1.0388355 | 1.0698605 | 0.9907107 | 0.8544527 | 0.9194536 | 1.0137987 |
| Phase-1 RCT-260 | 0.897414 | 1.0364053 | 1.1097759 | 0.9144571 | 0.889413 | 0.9390822 | 0.9712557 |
| Phase-1 RCT-12 | 1.2482727 | 1.1345938 | 1.0174124 | 0.9226162 | 0.9372356 | 1.0066543 | 0.9540671 |
| Phase-1 RCT-293 | 1.0360265 | 0.9689994 | 0.9197958 | 0.8696533 | 0.9880291 | 0.9197633 | 1.0190043 |
| Ribosomal protein S8 | 1.1281142 | 1.117432 | 0.972614 | 1.3226397 | 1.0485085 | 0.9735494 | 1.0957122 |
| Pyruvate kinase, muscle | 1.3151939 | 0.9010863 | 0.9711567 | 1.0526575 | 0.8318846 | 0.8387056 | 1.0147122 |
| Nucleoside diphosphate kinase beta isoform | 1.9878081 | 1.1362263 | 1.0725971 | 0.8808765 | 1.0048156 | 0.9885634 | 1.0429988 |
| p55CDC | 1.4895244 | 0.996911 | 1.0464994 | 0.9041542 | 0.6815164 | 0.8545544 | 0.8903958 |
| Phase-1 RCT-156 | 1.0311408 | 0.9207258 | 0.8891323 | 0.979939 | 1.0628765 | 1.166371 | 1.0607066 |
| Ribosomal protein L13A | 1.2517387 | 1.0101775 | 0.9963903 | 1.2506928 | 1.0689434 | 0.9183044 | 0.9043075 |
| Phase-1 RCT-258 | 0.9385621 | 1.0193295 | 1.0440072 | 1.001779 | 0.9734658 | 1.0004736 | 1.0861185 |
| Insulin-like growth factor I | 0.4700514 | 0.9188125 | 0.7727082 | 0.5511067 | 1.2376192 | 1.1373 | 1.4084662 |
| Cytochrome P450 3A1 | 0.6455215 | 0.8959016 | 0.3922329 | 0.5995183 | 0.8240973 | 0.6960103 | 0.6597287 |
| {Ribosomal protein 16} | 1.1473522 | 1.0273097 | 1.0375687 | 1.3193496 | 1.1288651 | 1.0076848 | 1.1071721 |
| Organic cation transporter 3 | 1.191203 | 0.9416404 | 0.9079131 | 1.2259693 | 0.987129 | 0.9759409 | 0.9431588 |
| Calpain 2 | 1.1870428 | 0.9604392 | 0.7851787 | 1.0625377 | 1.1041305 | 1.105531 | 1.0439684 |
| Phase-1 RCT-102 | 0.4632807 | 1.0768617 | 0.8093761 | 0.3142525 | 1.0229456 | 1.3454559 | 2.6082196 |
| Equilbrative nitrobenzylthioinosine-sensitive | 0.5969452 | 0.9039381 | 0.962787 | 0.5884379 | 1.1187575 | 1.0549179 | 1.054949 |
| nucleoside transporter | | | | | | | |
| Phase-1 RCT-227 | 0.647934 | 0.9224911 | 0.9632665 | 1.0354233 | 1.203295 | 1.0980854 | 0.9306343 |
| N-hydroxy-2-acetylaminofluorene sulfotransferase (ST1C1) | 0.3748783 | 0.8553504 | 0.7978133 | 0.5622116 | 1.041908 | 0.9947101 | 1.1540767 |
| Cathepsin S | 1.4410139 | 1.1142763 | 0.8698919 | 1.383046 | 1.1981723 | 0.9236499 | 1.0216403 |
| Apolipoprotein CIII | 0.6463311 | 0.7841811 | 1.0680707 | 0.785238 | 1.1693285 | 1.1413051 | 0.9944503 |
| Cytochrome P450 2A3 | 0.5056964 | 0.9763042 | 1.110723 | 0.971415 | 1.2974554 | 1.9072108 | 1.2701567 |
| Cytochrome P450 2C23 | 0.3641616 | 0.9741973 | 0.8632489 | 1.3367099 | 0.9427866 | 0.9247841 | 1.0541435 |
| Apolipoprotein C1 | 0.3911058 | 0.9302355 | 1.002663 | 0.9290003 | 1.1368703 | 1.0599985 | 0.9271419 |
| Betaine homocysteine methyltransferase (BHMT) | 0.623182 | 0.9653295 | 1.8382691 | 0.552529 | 2.2515023 | 1.9348925 | 1.3336868 |
| Paraoxonase 1 | 0.7337675 | 1.0564585 | 0.9426221 | 0.9864669 | 1.172843 | 1.1120679 | 1.2485526 |
| Phase-1 RCT-207 | 0.9898889 | 1.0023454 | 1.15641 | 1.0431026 | 0.8285792 | 0.9247772 | 1.0709904 |
| Cathepsin B | 1.6620054 | 1.0392579 | 1.0193175 | 1.2912853 | 1.3861555 | 1.293953 | 1.1172549 |
| Phase-1 RCT-144 | 1.1020281 | 1.2569329 | 1.151601 | 0.8738882 | 0.7696096 | 0.8912505 | 1.0473132 |
| Alpha-1 microglobulin/bikunin precursor (Ambp) | 0.642389 | 1.0245979 | 1.0553844 | 1.0075696 | 1.309138 | 1.2299662 | 1.1758246 |
| Cyclin D1 | 1.8199335 | 0.9044324 | 0.8486714 | 0.7077843 | 0.7941945 | 0.9096832 | 1.323637 |
| Presenilin-1 | 0.5280771 | 1.0400492 | 0.9699786 | 0.707888 | 1.1011269 | 0.7595058 | 1.1401119 |
| Protein tyrosine phosphatase, receptor type, D | 0.5207069 | 0.7764233 | 0.8604471 | 0.7184142 | 1.3878682 | 1.3847412 | 1.4572816 |
| Multidrug resistant protein-1 | 2.2996101 | 1.0857857 | 0.9745482 | 1.1220059 | 0.82151 | 0.9007906 | 0.8759366 |
| Cathepsin L, sequence 2 | 1.0532095 | 0.9516541 | 0.895649 | 1.5676248 | 0.832579 | 0.7402509 | 1.0790638 |
| JNK1 stress activated protein kinase | 0.9390989 | 0.9362173 | 1.1463608 | 1.356761 | 1.273815 | 1.0709795 | 0.8851773 |
| Phase-1 RCT-43 | 0.9903514 | 0.9998018 | 0.9956869 | 0.9475448 | 0.8792902 | 0.8088315 | 0.820267 |
| Tissue plasminogen activator | 1.0144669 | 1.2717144 | 0.9847027 | 0.5445492 | 0.9777468 | 0.969298 | 0.8875138 |
| Ubiquitin conjugating enzyme (RAD 6 | 1.1489016 | 1.2830906 | 0.9506218 | 1.0315295 | 1.0230616 | 1.0136826 | 1.2676282 |
| homologue) | | | | | | | |
| Phase-1 RCT-173 | 1.8110774 | 1.0483091 | 1.0929438 | 1.1953342 | 0.8763724 | 0.885724 | 0.9458615 |
| Ornithine decarboxylase | 1.2352933 | 1.2413557 | 1.2183381 | 0.7594518 | 0.8584034 | 1.1467687 | 1.2743678 |
| Zinc finger protein | 1.0319549 | 1.0139999 | 1.0155241 | 1.2443813 | 0.7828931 | 0.8186238 | 0.9447805 |
| CDK108 | 1.0901169 | 1.003766 | 0.7750834 | 0.7192235 | 1.0457361 | 1.0806737 | 0.8685312 |
| Phase-1 RCT-117 | 0.8885128 | 0.9365717 | 0.9073589 | 0.8242948 | 1.3425314 | 1.1512595 | 1.2047337 |
| {Phase-1 RCT 98} | 0.9531044 | 0.7963257 | 0.9300509 | 0.8135322 | 1.0398902 | 1.013246 | 0.9646019 |
| Aquaporin-3 (AQP3) | 0.8825231 | 0.9395661 | 1.0041261 | 0.982564 | 0.9389656 | 1.0063629 | 0.8272347 |
| Cholesterol esterase | 0.7746772 | 0.6884319 | 0.6288328 | 0.694101 | 0.8642439 | 0.9220865 | 0.7743779 |
| Ribosomal protein S17 | 1.2129514 | 1.1587939 | 1.0085871 | 1.3263733 | 0.9099618 | 0.8692929 | 1.0401394 |
| Poly(ADP-ribose) polymerase | 1.255621 | 1.051136 | 1.2161175 | 0.82019 | 0.9141933 | 0.8637289 | 0.8101997 |
| Annexin V | 1.2895945 | 1.0241947 | 1.0575234 | 0.9063979 | 0.8614278 | 1.0373245 | 1.2310396 |
| Phase-1 RCT-33 | 0.6527383 | 1.1576717 | 1.3540152 | 0.9613797 | 1.2850245 | 1.4761702 | 1.0214087 |
| Phase-1 RCT-61 | 1.192642 | 1.2510753 | 1.0264007 | 1.1690414 | 0.8929542 | 0.8706653 | 0.8548618 |
| Retinol dehydrogenase type III | 0.5850177 | 0.8846248 | 0.9694797 | 0.818899 | 1.4106495 | 1.4484183 | 1.2011015 |
| ATPase inhibitor (rat mitochondrial IF1 protein) | 0.3183253 | 0.6960456 | 0.7202776 | 0.5352186 | 1.2244457 | 1.0648046 | 1.2583281 |
| Thymidylate synthase | 1.1879234 | 1.0137469 | 1.0321711 | 1.3772736 | 0.914099 | 0.9245093 | 0.9076279 |
| Interleukin-18 | 1.2305624 | 0.8496136 | 0.9470511 | 1.031744 | 0.8088739 | 0.8997409 | 0.874535 |
| Lecithin: cholesterol acyltransferase | 0.7377166 | 1.0988829 | 0.9401532 | 1.0302929 | 1.2545502 | 1.1930611 | 1.1968971 |
| Contrapsin-like protease inhibitor (CPi-21) | 0.4180766 | 0.9095819 | 0.784817 | 0.2977954 | 1.1512234 | 0.955675 | 0.8719437 |
| Proliferating cell nuclear antigen gene | 1.2480869 | 0.8692914 | 0.9097262 | 0.8176634 | 0.7387173 | 0.7476562 | 0.7602304 |
| Phase-1 RCT-230 | 1.2919513 | 1.0521189 | 1.0424255 | 1.1047841 | 0.8037633 | 0.8097668 | 0.9028652 |
| Cytochrome P450 2D18 | 0.6960898 | 1.1316547 | 1.0301894 | 1.0929432 | 1.0840254 | 1.022659 | 0.8365338 |
| Phase-1 RCT-48 | 0.7699011 | 0.9601976 | 1.0362524 | 0.6062655 | 1.1676474 | 1.3600425 | 1.2040712 |
| Phase-1 RCT-292 | 0.8163309 | 0.9301136 | 0.9325755 | 1.0819685 | 1.1495725 | 1.0879095 | 0.9697109 |
| Arginosuccinate synthetase 1 | 0.4037345 | 0.8848568 | 1.9696424 | 1.2350507 | 1.5225463 | 1.6786261 | 1.0715278 |
| C-reactive protein | 0.5736723 | 1.0609636 | 0.8908129 | 0.7420979 | 1.4693254 | 1.4315186 | 1.6035335 |

| | CHEX | CHEX | CHEX | CHLOR | CHLOR | CHLOR |
|---|---|---|---|---|---|---|
| Compound-Dose | 2 | 2 | 2 | 8 | 8 | 8 |
| Animal Number | 2257 | 2258 | 2259 | 47 | 48 | 49 |
| Liver Toxicity Necrosis Classification | no | no | no | no | no | no |
| Gene Name | | | | | | |
| Osteoactivin | 0.9702393 | 1.1139009 | 0.9786929 | 1.0798992 | 1.1571282 | 1.2298236 |
| Stathmin | 0.8843797 | 1.1284696 | 0.9405939 | 1.2863133 | 1.1709479 | 1.058409 |
| Calpactin I heavy chain | 1.1113567 | 1.2168094 | 1.1562248 | 0.9570017 | 1.1146849 | 1.1739633 |
| Phase-1 RCT-179 | 0.9232199 | 1.013628 | 1.2365265 | 0.9573239 | 0.7987355 | 0.9357208 |
| Phase-1 RCT-107 | 1.005112 | 1.0281882 | 1.2244824 | 1.4641722 | 1.4848769 | 1.1046603 |
| Heme oxygenase | 1.0755113 | 0.9562546 | 1.0263407 | 0.708628 | 0.6417136 | 0.8349862 |
| Collagen type II | 0.4674191 | 0.6052859 | 0.5587537 | 1.2695647 | 1.3091512 | 1.1502279 |
| Phase-1 RCT-81 | 1.173966 | 1.0975692 | 1.415412 | 0.9945584 | 0.9461369 | 0.8308981 |
| Glycine methyltransferase | 1.1779883 | 1.1067345 | 3.1297202 | 2.1220722 | 1.5854284 | 1.1641561 |
| Cystatin C | 1.1930461 | 1.3474911 | 1.3913218 | 0.754284 | 0.7260702 | 0.689378 |
| Melanoma-associated antigen ME491 | 1.2463166 | 1.1764106 | 1.2536064 | 0.9561176 | 0.8630897 | 0.8692262 |
| Phase-1 RCT-146 | 0.6748213 | 0.9926668 | 0.7639465 | 0.989014 | 0.932836 | 0.9908797 |
| Gamma-actin, cytoplasmic | 1.2757019 | 1.0977464 | 1.1278372 | 1.0354893 | 0.9488661 | 1.3606876 |
| Adenine nucleotide translocator 1 | 0.700123 | 0.8731233 | 0.7788152 | 1.079754 | 1.2365897 | 1.0320692 |
| Thymosin beta-10 | 1.1430914 | 1.1281111 | 1.2807393 | 1.0164143 | 0.9654376 | 0.8240219 |
| Insulin-like growth factor I, exon 6 | 1.0329089 | 0.8405347 | 1.4050672 | 2.514955 | 1.6484292 | 1.9607165 |
| Phase-1 RCT-78 | 1.0966021 | 0.935558 | 1.0412034 | 0.9780869 | 0.9605923 | 0.9286752 |
| Alpha-2-microglobulin | 0.9010198 | 0.6836799 | 1.0169854 | 0.6193331 | 0.5163063 | 0.5934079 |
| High affinity IgE receptor gamma chain | 0.9510576 | 1.0530497 | 0.9750758 | 0.7450756 | 0.5217728 | 0.4722422 |
| (FcERIgamma) | | | | | | |
| 60S ribosomal protein L6 | 1.3267484 | 1.1071496 | 1.3732027 | 0.9876803 | 1.0113938 | 0.9174951 |
| IgE binding protein | 1.1143292 | 1.2159576 | 1.1423489 | 0.9553028 | 1.0637972 | 1.0932478 |
| Phase-1 RCT-149 | 1.0181329 | 1.0430963 | 0.9711112 | 1.2000263 | 1.2163907 | 1.1637868 |
| Cytochrome P450 2C11 | 0.6999242 | 0.7967385 | 0.781109 | 0.8784671 | 0.63455 | 0.7623972 |
| Uncoupling protein 2 | 0.807777 | 1.0730325 | 0.9123311 | 0.9852091 | 1.05414 | 1.0051489 |
| Cofilin | 0.8380272 | 0.9877079 | 0.9775203 | 1.1739458 | 1.0208899 | 1.1252747 |
| Alpha-tubulin | 0.8628 | 1.0473093 | 0.8819606 | 1.5197116 | 1.5592544 | 1.2309694 |
| Stearyl-CoA desaturase, liver | 0.2938865 | 3.0649602 | 1.1879843 | 2.7234874 | 3.7808871 | 2.840686 |
| Glutathione S-transferase theta-1 | 1.0430552 | 1.027386 | 0.9711648 | 0.8672658 | 0.8964018 | 0.8195046 |
| alpha-1,2-fucosyltrans-ferase | 1.2488483 | 1.0537407 | 1.0626274 | 1.0479116 | 1.2912405 | 1.3168327 |
| Phase-1 RCT-138 | 1.3180547 | 1.244243 | 1.3062649 | 1.0280358 | 0.8969008 | 0.917357 |
| Phase-1 RCT-295 | 1.5553797 | 1.3160044 | 1.5970403 | 0.8114961 | 1.1524172 | 1.0871247 |
| Phase-1 RCT-164 | 0.904379 | 1.0196917 | 1.2043636 | 0.7676972 | 0.6079314 | 0.7490448 |
| Alpha-2-macroglobulin, sequence 2 | 1.1472211 | 1.0512943 | 0.8817224 | 0.9222906 | 0.7474673 | 1.1688747 |
| Biliverdin reductase | 0.9197833 | 1.0724921 | 1.0241973 | 0.7711099 | 1.0269293 | 0.8025396 |
| Phase-1 RCT-121 | 0.5603762 | 0.8184028 | 0.6837857 | 1.0240215 | 0.879729 | 0.8731684 |
| Dynamin-1 (D100) | 1.0459186 | 1.2497054 | 1.1079288 | 0.9827925 | 0.9800271 | 0.9069612 |
| Phase-1 RCT-192 | 0.7584279 | 1.0530338 | 0.8754136 | 0.9855005 | 0.9738004 | 1.0632883 |
| Fatty acid synthase | 0.9288003 | 2.054674 | 0.9396319 | 1.3824844 | 1.5452504 | 1.1413392 |
| Phase-1 RCT-211 | 1.3119198 | 1.1654947 | 1.5154033 | 0.8033768 | 0.9296892 | 0.9701589 |
| Ribosomal protein S9 | 1.3812014 | 1.1815242 | 1.5445998 | 0.6813505 | 0.7202396 | 0.6609641 |
| NADP-dependent isocitrate dehydrogenase, | 1.4043375 | 1.3164289 | 1.4644089 | 0.7268117 | 0.6928544 | 0.7873476 |
| cytosolic | | | | | | |
| Phase-1 RCT-57 | 0.8852059 | 0.9359033 | 1.0467585 | 1.5322475 | 1.8614565 | 1.741052 |
| Phase-1 RCT-36 | 0.8233472 | 0.961939 | 0.7935457 | 1.2804548 | 1.146147 | 1.2019925 |
| Apolipoprotein All | 1.1870284 | 0.8079885 | 1.1394935 | 1.0765606 | 1.0046504 | 0.9653257 |
| Phase-1 RCT-68 | 1.0351826 | 0.907409 | 0.9161184 | 1.1309636 | 1.2373157 | 1.131174 |
| Multidrug resistant protein-2 | 0.9557599 | 0.8085944 | 0.7806571 | 0.974259 | 1.5471196 | 1.3940147 |
| Phase-1 RCT-39 | 1.072698 | 0.8207358 | 0.5360732 | 0.9259542 | 1.0140028 | 1.4228201 |
| NIPK | 0.7832866 | 0.8441891 | 0.9110763 | 2.1119123 | 1.7236537 | 1.9322562 |
| F1-ATPase beta subunit | 1.6400479 | 1.4885587 | 1.668286 | 1.6013303 | 1.2894635 | 1.1671807 |
| Elongation factor-1 alpha | 1.6041715 | 1.3817579 | 1.8668568 | 1.3234924 | 1.3155167 | 1.1922518 |
| Phase-1 RCT-24 | 0.6352841 | 0.7671423 | 0.654576 | 1.1522604 | 1.3453372 | 1.1728342 |
| Beta-tubulin, class I | 1.1732299 | 1.4154205 | 1.2193633 | 0.763424 | 0.9007596 | 0.8338937 |
| 14-3-3 zeta | 1.1200279 | 1.3179392 | 1.0097362 | 0.7396647 | 1.2236114 | 1.0825241 |
| Alpha-prothymosin | 1.0905126 | 1.0311427 | 1.2047989 | 0.8890111 | 1.6613665 | 1.531237 |
| Phase-1 RCT-58 | 2.540321 | 1.2147838 | 2.0926368 | 0.7853377 | 0.6086038 | 0.6184896 |
| Phase-1 RCT-290 | 1.2342535 | 1.1901733 | 1.5388677 | 1.288759 | 1.8892931 | 1.2662978 |
| Transitional endoplasmic reticulum ATPase | 0.4546625 | 0.5905041 | 0.555136 | 1.0063434 | 0.9054377 | 0.8902512 |
| Beta-actin | 1.7272073 | 1.5402557 | 1.6383622 | 1.5020322 | 2.5021272 | 1.790281 |
| Thioredoxin-1 (Trx1) | 1.2525408 | 1.0799121 | 1.3415598 | 0.7202812 | 0.7736889 | 0.8102788 |
| Phase-1 RCT-174 | 0.9148029 | 1.1029284 | 1.1209289 | 0.8900743 | 0.9999327 | 0.8731785 |
| Beta-actin, sequence 2 | 1.7934695 | 1.4060475 | 1.4816014 | 1.1303623 | 1.0215905 | 1.116062 |
| Phase-1 RCT-109 | 0.4540822 | 0.5391783 | 0.5161623 | 1.097172 | 1.1376302 | 0.9957448 |
| Phase-1 RCT-154 | 0.746921 | 0.9863434 | 0.864158 | 0.782558 | 0.9314696 | 0.9315038 |
| Hypoxanthine-guanine | 0.8677269 | 1.2133619 | 1.0984155 | 1.3381667 | 1.469046 | 1.6557747 |
| phosphoribosyltransferase | | | | | | |
| Phase-1 RCT-213 | 0.8585514 | 1.0156226 | 0.9827769 | 1.168935 | 1.1878437 | 1.1750649 |
| Phase-1 RCT-260 | 0.8709334 | 0.7007741 | 0.569643 | 0.8243232 | 0.8017747 | 1.1495974 |
| Phase-1 RCT-12 | 0.7570608 | 0.9601271 | 0.6990109 | 0.8409038 | 1.089559 | 0.9356279 |
| Phase-1 RCT-293 | 1.0994581 | 1.1895646 | 1.2516011 | 0.9599637 | 0.9620106 | 0.9171872 |
| Ribosomal protein S8 | 1.3986988 | 1.1528349 | 1.6132594 | 0.7485257 | 0.8058868 | 0.7506855 |
| Pyruvate kinase, muscle | 1.1416768 | 0.9451076 | 1.0615854 | 0.8958266 | 0.9247074 | 0.876653 |
| Nucleoside diphosphate kinase beta isoform | 1.2143561 | 1.2150639 | 1.1958193 | 0.704084 | 0.9130091 | 0.8702327 |
| p55CDC | 0.5989652 | 0.7725676 | 0.5744194 | 1.3310978 | 1.0144697 | 0.9109989 |
| Phase-1 RCT-156 | 1.0753598 | 1.1165774 | 1.2155776 | 1.1086513 | 1.025024 | 0.9187415 |
| Ribosomal protein L13A | 1.1720923 | 1.1009039 | 1.2660726 | 1.0661944 | 1.3550946 | 1.2000957 |
| Phase-1 RCT-258 | 0.8682188 | 0.9696479 | 1 | 1.2284157 | 0.9761515 | 1.0372341 |
| Insulin-like growth factor I | 1.2903261 | 0.8916084 | 1.6552103 | 1.0005404 | 0.8356105 | 0.9847208 |
| Cytochrome P450 3A1 | 1.3502904 | 0.9395891 | 1.3154951 | 1.0052997 | 0.4211558 | 0.4554279 |
| {Ribosomal protein 16} | 1.4044673 | 1.1266532 | 1.4860098 | 1.2875998 | 1.4328765 | 1.2176985 |
| Organic cation transporter 3 | 0.9528843 | 0.9346701 | 0.938557 | 0.9036324 | 0.9446088 | 0.915661 |
| Calpain 2 | 1.1930413 | 1.1721839 | 1.1305563 | 1.1702267 | 1.3114647 | 1.1426039 |
| Phase-1 RCT-102 | 1.0754031 | 0.6315831 | 1.0815759 | 1.0542135 | 1.1059602 | 1.1167592 |
| Equilbrative nitrobenzylthioinosine-sensitive | 1.2130934 | 0.9884806 | 0.9911417 | 0.8112741 | 0.4793459 | 0.6133457 |
| nucleoside transporter | | | | | | |
| Phase-1 RCT-227 | 1.3006263 | 0.9652505 | 1.4617512 | 0.9152069 | 0.9644726 | 0.877337 |
| N-hydroxy-2-acetylaminofluorene sulfotransferase (ST1C1) | 1.1667142 | 0.9110986 | 1.324219 | 0.5179471 | 0.4375308 | 0.4997667 |
| Cathepsin S | 1.1430979 | 0.9843253 | 0.8768072 | 1.04435 | 0.7480076 | 0.7716864 |
| Apolipoprotein CIII | 0.8730357 | 1.0783564 | 1.1561671 | 0.9656715 | 0.7762855 | 0.6345982 |
| Cytochrome P450 2A3 | 1.1111544 | 0.8799105 | 0.7967778 | 1.2330889 | 0.7453512 | 0.9126474 |
| Cytochrome P450 2C23 | 1.1242874 | 0.9763322 | 1.4652312 | 0.92465 | 0.7854656 | 0.8913941 |
| Apolipoprotein C1 | 1.0947304 | 0.8912359 | 1.403748 | 0.6763668 | 0.4219794 | 0.3613508 |
| Betaine homocysteine methyltransferase (BHMT) | 1.5637244 | 1.4924613 | 2.3219001 | 1.3024938 | 1.8318632 | 1.2406244 |
| Paraoxonase 1 | 1.6595635 | 1.0943853 | 1.6095389 | 0.7281988 | 0.4706661 | 0.5955157 |
| Phase-1 RCT-207 | 0.6565212 | 1.0031072 | 0.857376 | 1.086831 | 1.1804194 | 1.1069808 |
| Cathepsin B | 1.7477636 | 1.4937544 | 1.6196752 | 1.4063147 | 1.1725069 | 1.2341777 |
| Phase-1 RCT-144 | 0.8092386 | 0.8913565 | 0.7848666 | 0.9272996 | 0.9327552 | 0.8917462 |
| Alpha-1 microglobulin/bikunin precursor (Ambp) | 1.2644727 | 1.1695609 | 1.595321 | 1.0083066 | 1.0017376 | 0.8399561 |
| Cyclin D1 | 0.7304733 | 0.7917159 | 0.6915445 | 1.182251 | 1.0578438 | 0.7820823 |
| Presenilin-1 | 1.3006299 | 0.791919 | 1.0357963 | 1.3945503 | 0.8586056 | 1.060371 |
| Protein tyrosine phosphatase, receptor type, D | 1.55193 | 1.2761177 | 2.0121646 | 0.8824567 | 0.6450846 | 0.7307585 |
| Multidrug resistant protein-1 | 0.9379168 | 0.8802062 | 0.7493398 | 1.5089449 | 2.2585268 | 1.3836774 |
| Cathepsin L, sequence 2 | 1.1735681 | 0.9091848 | 1.0334852 | 0.8987513 | 0.8774558 | 0.8615292 |
| JNK1 stress activated protein kinase | 1.036872 | 0.9682458 | 1.0578736 | 1.0467904 | 0.7478303 | 0.5678276 |
| Phase-1 RCT-43 | 0.593066 | 0.8264862 | 0.7044742 | 0.7920379 | 0.8976669 | 0.8637852 |
| Tissue plasminogen activator | 0.8666678 | 0.8712236 | 0.6516575 | 1.0894964 | 1.0941912 | 1.1032641 |
| Ubiquitin conjugating enzyme (RAD 6 | 1.016601 | 1.0319165 | 1.3948337 | 0.6195018 | 0.6639344 | 0.6448802 |
| homologue) | | | | | | |
| Phase-1 RCT-173 | 0.7984151 | 1.0756235 | 0.9421376 | 0.957581 | 1.0429658 | 0.9918989 |
| Ornithine decarboxylase | 1.041059 | 1.0730969 | 0.9564332 | 1.0172977 | 2.417808 | 2.2122674 |
| Zinc finger protein | 0.6529034 | 0.8046479 | 0.8074203 | 1.1985853 | 1.0929422 | 1.1255347 |
| CDK108 | 0.8146887 | 0.7489997 | 0.9377164 | 0.9687485 | 1.1028266 | 1.0882081 |
| Phase-1 RCT-117 | 0.6416434 | 1.2677867 | 1.4875454 | 1.2420787 | 1.0555042 | 1.132878 |
| {Phase-1 RCT 98} | 0.8313986 | 0.852252 | 0.8408684 | 1.8184929 | 1.3692408 | 1.2760043 |
| Aquaporin-3 (AQP3) | 0.7520178 | 0.8131149 | 0.6961234 | 0.9955567 | 1.0711558 | 1.025233 |
| Cholesterol esterase | 0.7250696 | 0.7321469 | 1.2694058 | 1.2160605 | 1.1812155 | 0.8609142 |
| Ribosomal protein S17 | 1.1812526 | 0.9803968 | 1.3308282 | 0.8192028 | 1.0181838 | 0.8843263 |
| Poly(ADP-ribose) polymerase | 0.8221334 | 0.9020714 | 0.7391129 | 1.2624797 | 1.3923514 | 1.4138901 |
| Annexin V | 1.0583758 | 1.0664667 | 1.0654664 | 1.0432085 | 0.9384557 | 0.8200424 |
| Phase-1 RCT-33 | 1.2594914 | 1.3221693 | 1.5965574 | 1.1969851 | 1.1416105 | 1.2288055 |
| Phase-1 RCT-61 | 0.63711 | 0.8325849 | 0.7865279 | 1.0348133 | 0.8335125 | 0.8726847 |
| Retinol dehydrogenase type III | 1.1703953 | 1.2584928 | 1.8120792 | 0.7551162 | 0.6284593 | 0.7576454 |
| ATPase inhibitor (rat mitochondrial IF1 protein) | 1.3436245 | 1.0702077 | 1.4736633 | 0.5871739 | 0.5110844 | 0.665899 |
| Thymidylate synthase | 0.7169592 | 0.8252109 | 0.672826 | 1.1047226 | 1.2136282 | 0.9007418 |
| Interleukin-18 | 0.5303662 | 0.6649682 | 0.6437922 | 0.8907131 | 0.8341022 | 0.8284617 |
| Lecithin: cholesterol acyltransferase | 1.4501469 | 1.2189181 | 1.5043184 | 1.0051241 | 1.1125736 | 1.1369778 |
| Contrapsin-like protease inhibitor (CPi-21) | 0.6822206 | 0.582888 | 0.9667429 | 0.9567837 | 1.0236998 | 1.0626938 |
| Proliferating cell nuclear antigen gene | 0.6197826 | 0.708785 | 0.6637777 | 1.0025455 | 0.856393 | 0.8505266 |
| Phase-1 RCT-230 | 0.915358 | 0.8057272 | 0.6712488 | 0.9142419 | 0.8307661 | 1.550924 |
| Cytochrome P450 2D18 | 1.3283215 | 1.1101375 | 1.4953625 | 1.5575929 | 1.8010932 | 1.620505 |
| Phase-1 RCT-48 | 1.0715406 | 0.8047096 | 1.2716805 | 1.3299919 | 1.3495058 | 1.208087 |
| Phase-1 RCT-292 | 1.0443529 | 1.0988458 | 1.1689665 | 1.1050242 | 1.096326 | 1.0773232 |
| Arginosuccinate synthetase 1 | 1.0137787 | 1.1707187 | 1.47527 | 1.3367257 | 1.3967797 | 1.0883938 |
| C-reactive protein | 1.628257 | 1.359493 | 2.0878046 | 0.5024643 | 0.567119 | 0.602936 |

| | CHLOR | CHLOR | CHLOR | CIS | CIS | CIS | CIS |
|---|---|---|---|---|---|---|---|
| Compound-Dose | 30 | 30 | 30 | 2.5 | 2.5 | 2.5 | 10 |
| Animal Number | 57 | 58 | 59 | 327 | 328 | 329 | 337 |
| Liver Toxicity Necrosis Classification | no | no | no | no | no | no | no |
| Gene Name | | | | | | | |
| Osteoactivin | 0.9869648 | 0.874861 | 1.273223 | 1.0045757 | 1.0052737 | 0.9458963 | 1.1182581 |
| Stathmin | 1.078137 | 0.9977121 | 1.2240943 | 0.8710316 | 0.9282308 | 0.9803675 | 0.9191238 |
| Calpactin I heavy chain | 1.2326566 | 1.2145594 | 1.1363053 | 1.044323 | 1.1516588 | 1.0419444 | 0.8249402 |
| Phase-1 RCT-179 | 0.8116236 | 0.8431138 | 1.0556914 | 1.0046568 | 1.008543 | 0.9234892 | 0.8943745 |
| Phase-1 RCT-107 | 1.3795502 | 1.3559662 | 0.9980943 | 1.3744085 | 0.9838737 | 0.8566032 | 1.2055279 |
| Heme oxygenase | 0.956463 | 0.6850266 | 0.6451342 | 1.0972974 | 0.7247987 | 0.7239071 | 0.6515169 |
| Collagen type II | 1.1150538 | 1.2754283 | 0.8932005 | 0.9162589 | 1.0397073 | 0.896785 | 0.8749799 |
| Phase-1 RCT-81 | 0.8221971 | 0.8437587 | 0.9230817 | 1.0348674 | 0.9956718 | 0.9813101 | 0.9318418 |
| Glycine methyltransferase | 1.353086 | 1.4021288 | 1.040358 | 3.0997126 | 1.0690762 | 1.3857857 | 1.911817 |
| Cystatin C | 0.8782346 | 0.8056421 | 0.7665951 | 0.8566671 | 0.7771387 | 0.8301089 | 0.8072313 |
| Melanoma-associated antigen ME491 | 1.0674677 | 1.0251284 | 1.0768979 | 1.406844 | 0.8753864 | 1.1619064 | 0.9067785 |
| Phase-1 RCT-146 | 1.13651 | 1.0848345 | 0.9956114 | 1.0772121 | 1.053466 | 1.1527086 | 1.027553 |
| Gamma-actin, cytoplasmic | 1.217573 | 1.2618349 | 1.1896404 | 1.0468225 | 0.8113672 | 1.0323488 | 0.8723598 |
| Adenine nucleotide translocator 1 | 0.7902642 | 0.7863472 | 0.9314933 | 0.9325614 | 0.9396254 | 0.9110413 | 0.9315002 |
| Thymosin beta-10 | 1.3058318 | 1.2772346 | 0.9980302 | 1.1307868 | 1.0323272 | 1.0620955 | 1.0117003 |
| Insulin-like growth factor I, exon 6 | 1.3635079 | 1.1093729 | 1.3781426 | 0.7898039 | 0.7734144 | 0.7844001 | 0.6008679 |
| Phase-1 RCT-78 | 0.9764966 | 1.0568516 | 1.1187435 | 0.8495624 | 0.9233494 | 0.9314135 | 0.7371724 |
| Alpha-2-microglobulin | 0.4863668 | 0.5405361 | 0.5516049 | 0.9008522 | 0.7761901 | 0.9322268 | 0.2029774 |
| High affinity IgE receptor gamma chain | 0.6936925 | 0.672524 | 0.6550245 | 0.8415254 | 0.8957897 | 0.8361509 | 0.7321135 |
| (FcERIgamma) | | | | | | | |
| 60S ribosomal protein L6 | 1.000155 | 0.9609194 | 0.9965736 | 0.9938114 | 1.0258949 | 1.0444078 | 0.9691675 |
| IgE binding protein | 1.0280224 | 1.0396687 | 1.1877469 | 0.9123824 | 0.9788468 | 0.9274033 | 0.8506749 |
| Phase-1 RCT-149 | 1.0530714 | 1.1058201 | 1.2165977 | 1.0448428 | 1.0612918 | 1.0175226 | 2.0141788 |
| Cytochrome P450 2C11 | 1.2045391 | 1.2962492 | 1.0154575 | 1.4810356 | 1.0964901 | 1.3281355 | 0.8200362 |
| Uncoupling protein 2 | 1.4297596 | 1.3435005 | 1.059544 | 1.0399705 | 0.9775778 | 0.9259909 | 0.8963276 |
| Cofilin | 0.8372086 | 0.9027177 | 1.2045374 | 1.0472926 | 1.1864699 | 1.0473363 | 0.9916496 |
| Alpha-tubulin | 1.3144951 | 1.1400461 | 1.0270818 | 1.0623167 | 1.0162864 | 0.8925532 | 0.6456676 |
| Stearyl-CoA desaturase, liver | 2.6756694 | 4.12825 | 3.2845416 | 0.5959176 | 1.927161 | 0.5011968 | 0.1480728 |
| Glutathione S-transferase theta-1 | 0.8333892 | 0.8275917 | 0.9421158 | 1.2502565 | 1.6064329 | 0.9507136 | 0.6872602 |
| alpha-1,2-fucosyltrans-ferase | 1.9527545 | 1.7721143 | 1.3910074 | 0.7753043 | 0.7794209 | 0.8237816 | 0.7350845 |
| Phase-1 RCT-138 | 1.0651504 | 1.0469321 | 1.0549402 | 0.9604106 | 0.9814834 | 0.9223368 | 0.9474628 |
| Phase-1 RCT-295 | 1.2721123 | 1.365131 | 1.2487458 | 0.9022591 | 0.9375227 | 0.9516865 | 0.874212 |
| Phase-1 RCT-164 | 0.8588073 | 0.8356723 | 0.8086121 | 1.1766024 | 1.0593404 | 1.0946931 | 1.5542841 |
| Alpha-2-macroglobulin, sequence 2 | 1.2118565 | 1.1515374 | 1.2663665 | 1.0743922 | 1.1319952 | 1.1076765 | 1.588947 |
| Biliverdin reductase | 1.1320002 | 1.9617624 | 1.009767 | 0.9758207 | 1.0025079 | 1.149612 | 0.859721 |
| Phase-1 RCT-121 | 0.7985507 | 0.7993821 | 0.8562684 | 0.9695765 | 0.976125 | 0.9425035 | 1.0946791 |
| Dynamin-1 (D100) | 1.0890479 | 1.273531 | 1.0949918 | 0.9625475 | 0.9999253 | 0.9205288 | 1.0457985 |
| Phase-1 RCT-192 | 1.0352718 | 0.9121471 | 1.0899324 | 0.9649649 | 1.0301207 | 0.8978589 | 0.8341894 |
| Fatty acid synthase | 0.9709938 | 1.4400104 | 1.0490482 | 0.5576518 | 1.6281012 | 0.7240897 | 0.4473033 |
| Phase-1 RCT-211 | 1.3696262 | 1.3212457 | 0.5639697 | 0.8548513 | 0.8457502 | 0.8398795 | 1.0528066 |
| Ribosomal protein S9 | 0.8009931 | 0.799509 | 0.845456 | 0.9807387 | 1.1086347 | 1.043815 | 0.9585394 |
| NADP-dependent isocitrate dehydrogenase, | 0.8051905 | 0.8420598 | 0.9175521 | 0.8415921 | 0.9788343 | 1.0502692 | 0.8050497 |
| cytosolic | | | | | | | |
| Phase-1 RCT-57 | 1.6115066 | 1.4140458 | 1.5683655 | 0.9798355 | 1.1733254 | 1.2176801 | 1.3357265 |
| Phase-1 RCT-36 | 1.3733587 | 1.5653988 | 1.3046234 | 1.026814 | 0.8612306 | 0.9126741 | 0.9596457 |
| Apolipoprotein All | 2.0034823 | 2.102714 | 1.5083909 | 0.6963546 | 0.4132561 | 0.8210438 | 0.4703139 |
| Phase-1 RCT-68 | 1.1271183 | 1.2439942 | 1.115934 | 1.0702885 | 0.9730849 | 0.9851946 | 1.2067306 |
| Multidrug resistant protein-2 | 0.9547172 | 1.1311151 | 0.8280652 | 1.4526156 | 1.2765114 | 1.1861275 | 4.9602737 |
| Phase-1 RCT-39 | 1.3719547 | 1.2500061 | 0.9851612 | 1.01714 | 0.928345 | 0.8545149 | 1.0281411 |
| NIPK | 1.3373113 | 1.113767 | 1.4045756 | 0.9616525 | 0.9327519 | 0.9486475 | 1.1049502 |
| F1-ATPase beta subunit | 0.7567053 | 0.8581148 | 0.954472 | 0.9074919 | 0.8797525 | 0.9262494 | 0.936627 |
| Elongation factor-1 alpha | 1.040813 | 1.2150749 | 1.4475971 | 1.1277368 | 1.1032752 | 1.0376132 | 0.8566524 |
| Phase-1 RCT-24 | 1.3492743 | 1.2743391 | 1.0439159 | 1.0577207 | 1.128349 | 0.8995729 | 0.7073689 |
| Beta-tubulin, class I | 1.0662631 | 0.9818872 | 0.8080024 | 1.2305417 | 1.4083989 | 0.9479913 | 0.7040052 |
| 14-3-3 zeta | 1.2542893 | 1.1564059 | 0.8531098 | 1.0202873 | 1.0055805 | 0.9615413 | 0.9331554 |
| Alpha-prothymosin | 1.067011 | 1.2604926 | 1.6659577 | 0.8537823 | 1.0379025 | 1.0150802 | 0.5957414 |
| Phase-1 RCT-58 | 0.6121987 | 0.5942719 | 0.7638408 | 0.9393575 | 1.3544979 | 0.753568 | 0.9020432 |
| Phase-1 RCT-290 | 1.0918809 | 2.0421503 | 1.0452602 | 1.3182094 | 1.1283954 | 1.1438283 | 1.5195469 |
| Transitional endoplasmic reticulum ATPase | 0.7239874 | 0.7764758 | 0.7909877 | 1.1043353 | 1.134709 | 1.0096718 | 0.9450783 |
| Beta-actin | 1.3869315 | 1.5121499 | 1.0937084 | 0.9520429 | 0.7971798 | 0.9004195 | 0.6332651 |
| Thioredoxin-1 (Trx1) | 0.9675171 | 0.9506312 | 0.8500686 | 0.8893212 | 0.9577922 | 1.0050716 | 0.7116818 |
| Phase-1 RCT-174 | 1.0342609 | 1.2204137 | 1.2404741 | 1.0896472 | 1.0841581 | 1.0337051 | 0.8470991 |
| Beta-actin, sequence 2 | 0.8237315 | 0.9290008 | 1.1983411 | 0.5998557 | 0.5109926 | 0.621711 | 0.4579818 |
| Phase-1 RCT-109 | 1.1748916 | 1.3785454 | 1.0950263 | 0.8250554 | 0.8415988 | 0.7643202 | 0.7603682 |
| Phase-1 RCT-154 | 0.9614874 | 1.0030313 | 1.1311677 | 0.988833 | 1.1784314 | 1.4258482 | 1.6071781 |
| Hypoxanthine-guanine | 1.0716854 | 1.1988466 | 1.0600398 | 0.9808455 | 1.1388215 | 1.0063814 | 1.0344555 |
| phosphoribosyltransferase | | | | | | | |
| Phase-1 RCT-213 | 1.165072 | 1.1004646 | 0.9290667 | 1.0054494 | 1.0625972 | 1.0682346 | 0.994459 |
| Phase-1 RCT-260 | 1.1014438 | 0.8688564 | 0.8239837 | 1.3128158 | 0.9607818 | 1.0382489 | 1.1121787 |
| Phase-1 RCT-12 | 1.3177853 | 1.215746 | 1.0763367 | 1.026814 | 1.0131669 | 0.8390024 | 0.9678524 |
| Phase-1 RCT-293 | 2.0717518 | 1.5075413 | 1.4115978 | 0.8974685 | 1.0079584 | 0.9998678 | 0.8333895 |
| Ribosomal protein S8 | 0.9298846 | 0.9891706 | 1.0299382 | 1.0786959 | 1.0726333 | 0.9038857 | 0.9130253 |
| Pyruvate kinase, muscle | 1.0712438 | 0.9788088 | 0.9234497 | 1.2100815 | 0.969047 | 0.9911111 | 1.2756343 |
| Nucleoside diphosphate kinase beta isoform | 1.068265 | 0.9283232 | 0.7980885 | 1.0548846 | 1.1322806 | 1.0042723 | 0.9791521 |
| p55CDC | 0.8723404 | 0.6865102 | 1.0827497 | 1.0640508 | 0.8578295 | 1.1023355 | 0.9122131 |
| Phase-1 RCT-156 | 0.8175409 | 0.8660827 | 0.9578367 | 0.8612908 | 0.9814929 | 0.8027278 | 0.8497742 |
| Ribosomal protein L13A | 1.6335803 | 1.3975961 | 1.286034 | 0.8126704 | 0.8442828 | 0.7867758 | 0.7102276 |
| Phase-1 RCT-258 | 0.9064292 | 0.7949979 | 0.966536 | 0.9788032 | 0.9952081 | 0.9369644 | 0.9314758 |
| Insulin-like growth factor I | 0.5636259 | 0.6651205 | 0.8841587 | 0.8446736 | 0.7896087 | 0.7366254 | 0.5471235 |
| Cytochrome P450 3A1 | 0.6476187 | 0.6565121 | 0.6604551 | 1.2221346 | 0.9384432 | 0.9617313 | 0.2492068 |
| {Ribosomal protein 16} | 1.253239 | 1.6449294 | 1.3278698 | 0.9578304 | 1.0378847 | 0.8988153 | 0.8761715 |
| Organic cation transporter 3 | 1.0020739 | 1.0025845 | 1.0150684 | 0.9501709 | 0.9810325 | 0.8892357 | 1.1004934 |
| Calpain 2 | 1.3093318 | 1.4787533 | 1.3528547 | 1.0079929 | 1.0187578 | 0.9198151 | 0.9732453 |
| Phase-1 RCT-102 | 0.6138431 | 0.9075422 | 0.8654161 | 0.8490434 | 0.6650832 | 0.5583635 | 0.382954 |
| Equilbrative nitrobenzylthioinosine-sensitive | 0.6603369 | 0.5281606 | 0.627035 | 1.2279987 | 1.0335342 | 1.0669224 | 0.6289416 |
| nucleoside transporter | | | | | | | |
| Phase-1 RCT-227 | 0.9885414 | 1.1154537 | 1.1470227 | 0.8526102 | 0.8281975 | 1.4431889 | 0.6307741 |
| N-hydroxy-2-acetylaminofluorene sulfotransferase (ST1C1) | 0.5729623 | 0.5614737 | 0.6174232 | 1.045023 | 0.9228173 | 0.8236575 | 0.2425152 |
| Cathepsin S | 0.9173963 | 0.8592099 | 1.0205146 | 0.9101036 | 0.6704568 | 0.7873159 | 0.8413535 |
| Apolipoprotein CIII | 0.7568777 | 0.8064852 | 0.7821947 | 1.1028612 | 1.2394449 | 1.1380144 | 1.3020456 |
| Cytochrome P450 2A3 | 0.7822909 | 0.947762 | 1.2104001 | 1.1409347 | 0.8079592 | 0.7939636 | 1.2608137 |
| Cytochrome P450 2C23 | 0.8131651 | 0.8117639 | 0.912873 | 1.0025927 | 1.0532854 | 0.8362247 | 0.9881074 |
| Apolipoprotein C1 | 0.5502043 | 0.5663 | 0.5768477 | 1.1080104 | 0.9438011 | 1.1849256 | 0.8075554 |
| Betaine homocysteine methyltransferase (BHMT) | 1.0751051 | 1.7301015 | 1.1030817 | 1.28565 | 1.3552231 | 1.2263014 | 2.003289 |
| Paraoxonase 1 | 0.4811081 | 0.7051384 | 0.8312656 | 0.9204702 | 0.8090153 | 0.9116302 | 0.5435456 |
| Phase-1 RCT-207 | 1.1393545 | 1.1142051 | 0.9087126 | 1.1143657 | 1.2271461 | 1.2290782 | 2.4369507 |
| Cathepsin B | 1.0745515 | 0.9731555 | 1.077813 | 0.8980445 | 1.000247 | 0.9689227 | 1.1264436 |
| Phase-1 RCT-144 | 0.9927374 | 1.0225378 | 1.0252868 | 0.9146091 | 0.9581392 | 0.9061182 | 0.8721893 |
| Alpha-1 microglobulin/bikunin precursor (Ambp) | 0.8412198 | 0.8851407 | 0.9798541 | 1.042535 | 1.0968413 | 1.0188049 | 0.9837073 |
| Cyclin D1 | 0.5014579 | 0.642071 | 0.745113 | 0.7302305 | 1.2339196 | 0.9660044 | 0.8899934 |
| Presenilin-1 | 0.6211982 | 0.6327217 | 1.2118648 | 1.015062 | 0.9073956 | 0.8695355 | 0.3900196 |
| Protein tyrosine phosphatase, receptor type, D | 0.8399901 | 0.8772157 | 0.6633298 | 0.9555025 | 1.1499182 | 1.1026882 | 1.0931877 |
| Multidrug resistant protein-1 | 1.465797 | 1.632823 | 1.046808 | 1.6463925 | 1.5136085 | 1.339567 | 7.1580973 |
| Cathepsin L, sequence 2 | 0.9682182 | 0.856351 | 0.8732222 | 1.1819208 | 0.9380166 | 0.9913082 | 1.2295662 |
| JNK1 stress activated protein kinase | 0.9782964 | 1.0721385 | 0.7660409 | 1.3321251 | 1.0617193 | 1.3050778 | 1.3835706 |
| Phase-1 RCT-43 | 0.7885317 | 0.8540376 | 1.0034727 | 0.8886287 | 0.9652312 | 0.8788087 | 1.2132287 |
| Tissue plasminogen activator | 0.7850498 | 0.9558322 | 1.1758537 | 0.9071759 | 0.9383342 | 0.8794575 | 0.7172432 |
| Ubiquitin conjugating enzyme (RAD 6 | 0.7720608 | 0.7759548 | 0.8322064 | 1.0563993 | 1.0078319 | 1.0030812 | 1.0160139 |
| homologue) | | | | | | | |
| Phase-1 RCT-173 | 1.0117295 | 0.9951286 | 0.9383324 | 0.967313 | 1.2230926 | 0.938926 | 0.9437677 |
| Ornithine decarboxylase | 2.436342 | 2.1822882 | 1.6488656 | 1.0758299 | 1.2340997 | 1.100724 | 1.3363043 |
| Zinc finger protein | 0.8306956 | 0.7912155 | 1.1229216 | 1.0573537 | 1.0604855 | 1.0071181 | 1.1038026 |
| CDK108 | 1.1770562 | 1.084764 | 0.9766247 | 1.2341902 | 1.0652301 | 1.2394243 | 0.7980717 |
| Phase-1 RCT-117 | 1.3564394 | 0.8487007 | 0.924093 | 0.8717595 | 0.9649923 | 1.0729719 | 0.9323651 |
| {Phase-1 RCT 98} | 0.8729877 | 0.9032333 | 0.9584088 | 0.9956983 | 0.8693985 | 0.9625843 | 1.0712806 |
| Aquaporin-3 (AQP3) | 0.9996985 | 1.0529689 | 1.0655448 | 0.9957577 | 0.9145036 | 0.8474362 | 1.0010842 |
| Cholesterol esterase | 0.5638902 | 0.5492982 | 0.6525343 | 1.1883056 | 0.9688797 | 0.9201106 | 0.2536994 |
| Ribosomal protein S17 | 1.7802523 | 1.462145 | 1.3874545 | 1.0616877 | 1.1004155 | 0.92796 | 0.890899 |
| Poly(ADP-ribose) polymerase | 1.2570182 | 1.2958785 | 1.1166209 | 0.9685372 | 1.0537622 | 0.9870325 | 0.8264679 |
| Annexin V | 0.5290656 | 0.6564416 | 0.9804512 | 0.972618 | 0.9868605 | 1.0157284 | 0.9315673 |
| Phase-1 RCT-33 | 1.3133556 | 1.2810203 | 1.1169575 | 0.800114 | 0.9499743 | 1.0281783 | 0.7955064 |
| Phase-1 RCT-61 | 0.9260918 | 1.1318198 | 1.2932565 | 0.9228319 | 1.1067811 | 0.9209172 | 1.2266287 |
| Retinol dehydrogenase type III | 0.8956609 | 0.8302253 | 0.8281531 | 1.3155967 | 1.10271 | 1.2432023 | 1.0921876 |
| ATPase inhibitor (rat mitochondrial IF1 protein) | 0.8738809 | 0.8041205 | 0.7800946 | 1.0799079 | 0.9519141 | 0.9855038 | 0.4833648 |
| Thymidylate synthase | 1.1466669 | 0.9241809 | 1.502107 | 1.1226279 | 1.2650648 | 1.2070516 | 1.462114 |
| Interleukin-18 | 0.942134 | 0.9298005 | 0.9400477 | 1.0177032 | 1.0280795 | 1.2137519 | 1.0933931 |
| Lecithin: cholesterol acyltransferase | 1.1592296 | 1.0635988 | 0.9775313 | 0.7769228 | 0.9532017 | 1.0029329 | 1.4331611 |
| Contrapsin-like protease inhibitor (CPi-21) | 0.6662508 | 0.993955 | 1.1830107 | 0.9046449 | 0.5840787 | 0.7183877 | 0.1009068 |
| Proliferating cell nuclear antigen gene | 1.1399528 | 1.2204137 | 0.6917863 | 1.1063172 | 1.0461594 | 0.9053529 | 1.164426 |
| Phase-1 RCT-230 | 1.2828422 | 1.1635735 | 0.9295446 | 1.100251 | 0.9121606 | 0.9352149 | 0.9616602 |
| Cytochrome P450 2D18 | 1.6493651 | 1.3937049 | 1.5848188 | 1.1304383 | 0.992913 | 1.2354817 | 0.9226562 |
| Phase-1 RCT-48 | 1.0715171 | 1.2106082 | 1.4172335 | 0.8710354 | 0.698057 | 0.8740051 | 0.5653416 |
| Phase-1 RCT-292 | 1.135403 | 1.0933281 | 1.0464078 | 0.8957689 | 0.9870327 | 0.9878346 | 0.9944391 |
| Arginosuccinate synthetase 1 | 1.03359 | 1.5268266 | 1.2126695 | 1.0997972 | 1.2906413 | 1.0294539 | 1.990084 |
| C-reactive protein | 0.7145932 | 0.7865626 | 0.6521254 | 1.0010544 | 1.1012925 | 0.9968191 | 1.049562 |

| | CIS | CLO | CLO | CLO | CLO | CLO |
|---|---|---|---|---|---|---|
| Compound-Dose | 10 | 75 | 75 | 75 | 250 | 250 |
| Animal Number | 338 | 327 | 328 | 329 | 1847 | 1848 |
| Liver Toxicity Necrosis Classification | no | no | no | no | no | no |
| Gene Name | | | | | | |
| Osteoactivin | 1.1104393 | 0.9053036 | 0.9081503 | 1.0902715 | 0.8116155 | 0.8898287 |
| Stathmin | 0.7821424 | 1.2866173 | 1.1930366 | 1.2599263 | 1.1661102 | 1.1712971 |
| Calpactin I heavy chain | 0.8825272 | 1.1848924 | 1.1991482 | 1.2513227 | 1.1299608 | 1.2350848 |
| Phase-1 RCT-179 | 0.9263911 | 0.8827379 | 0.8725108 | 0.9515146 | 0.8913868 | 0.8809685 |
| Phase-1 RCT-107 | 1.1860067 | 0.9878053 | 0.9829438 | 1.0979954 | 0.8579626 | 1.1535496 |
| Heme oxygenase | 0.811832 | 1.4941287 | 1.1594455 | 1.2341359 | 1.2740924 | 1.0051638 |
| Collagen type II | 0.9227452 | 1.5041149 | 1.1606214 | 1.3658911 | 1.4748582 | 1.279971 |
| Phase-1 RCT-81 | 0.9530942 | 0.7464432 | 0.8136552 | 0.8398508 | 0.7687504 | 0.8524849 |
| Glycine methyltransferase | 2.5303175 | 0.8633941 | 0.9879577 | 1.1574805 | 0.6301502 | 1.1144465 |
| Cystatin C | 0.7966737 | 0.6775722 | 0.8173381 | 0.8727651 | 0.7090629 | 0.8142077 |
| Melanoma-associated antigen ME491 | 1.0484036 | 0.9489572 | 0.8796393 | 1.0958934 | 0.7993794 | 0.9692329 |
| Phase-1 RCT-146 | 1.1039728 | 1.2046663 | 1.0714042 | 1.2168474 | 1.1001357 | 1.0400429 |
| Gamma-actin, cytoplasmic | 0.9772505 | 0.7362058 | 0.8494061 | 0.7884935 | 1.005585 | 0.8131885 |
| Adenine nucleotide translocator 1 | 0.8666508 | 0.7078828 | 0.8168544 | 0.8097797 | 0.846294 | 0.8266118 |
| Thymosin beta-10 | 1.0462619 | 1.2527415 | 1.0449291 | 1.247654 | 1.1098052 | 1.1831346 |
| Insulin-like growth factor I, exon 6 | 0.8080932 | 0.8659487 | 1.0503792 | 0.900646 | 1.1516336 | 0.919231 |
| Phase-1 RCT-78 | 0.737639 | 0.7675977 | 0.7743206 | 0.8883135 | 0.8343665 | 0.8398009 |
| Alpha-2-microglobulin | 0.8873122 | 0.6022567 | 1.0304253 | 1.0774782 | 0.9473388 | 1.1446697 |
| High affinity IgE receptor gamma chain | 0.8595916 | 0.8462859 | 0.8629695 | 1.0338173 | 0.8750328 | 0.9355069 |
| (FcERIgamma) | | | | | | |
| 60S ribosomal protein L6 | 0.9712295 | 0.8915091 | 0.952731 | 0.9695599 | 0.9214662 | 0.8867597 |
| IgE binding protein | 1.0313941 | 1.0740477 | 1.168987 | 1.1904484 | 1.0164793 | 1.0575904 |
| Phase-1 RCT-149 | 1.2074429 | 1.2154263 | 1.2481866 | 1.23539 | 1.1345481 | 1.3323009 |
| Cytochrome P450 2C11 | 0.7488598 | 1.0649015 | 1.0341321 | 1.3738436 | 1.3041102 | 1.2884976 |
| Uncoupling protein 2 | 0.8876261 | 1.4395825 | 1.0585432 | 1.277636 | 1.2234535 | 1.1503826 |
| Cofilin | 0.9999999 | 0.6650038 | 0.8277907 | 0.9578929 | 0.8686636 | 0.934597 |
| Alpha-tubulin | 0.7091392 | 1.1700181 | 1.1157101 | 1.1361918 | 1.2718383 | 1.1317111 |
| Stearyl-CoA desaturase, liver | 0.0450286 | 1.7237905 | 2.6701481 | 1.0577376 | 0.9531848 | 1.2207347 |
| Glutathione S-transferase theta-1 | 0.7580161 | 1.0542271 | 1.1357192 | 1.0091136 | 1.137063 | 0.9253646 |
| alpha-1,2-fucosyltrans-ferase | 0.6614928 | 1.6028955 | 1.6189716 | 1.5326133 | 1.5009662 | 1.4460781 |
| Phase-1 RCT-138 | 1.0708345 | 0.8801588 | 0.9536248 | 0.9959633 | 0.8610631 | 0.9192557 |
| Phase-1 RCT-295 | 0.8707937 | 0.9135225 | 1.0787352 | 1.0468419 | 0.9989374 | 0.9973521 |
| Phase-1 RCT-164 | 0.8149999 | 1.1929319 | 1.0539793 | 1.0550326 | 1.0061692 | 1.3746598 |
| Alpha-2-macroglobulin, sequence 2 | 1.0858897 | 0.6682603 | 0.8175379 | 0.8718258 | 0.8302919 | 0.8381426 |
| Biliverdin reductase | 0.9698 | 1.0727723 | 1.0841044 | 1.1548634 | 1.1595095 | 1.2131915 |
| Phase-1 RCT-121 | 0.8280185 | 1.3056028 | 0.9329193 | 1.0641267 | 1.064165 | 1.0115452 |
| Dynamin-1 (D100) | 1.0395935 | 0.8043146 | 0.9282762 | 0.9725013 | 0.8988914 | 0.9690961 |
| Phase-1 RCT-192 | 0.9661005 | 0.8657568 | 0.9478765 | 0.9505863 | 0.8299891 | 0.8880065 |
| Fatty acid synthase | 0.3963719 | 1.4151032 | 1.7867988 | 0.6657438 | 0.6746896 | 0.8745828 |
| Phase-1 RCT-211 | 1.1025147 | 1.3528906 | 1.46164 | 1.2143196 | 1.186814 | 1.2653166 |
| Ribosomal protein S9 | 0.9467907 | 0.839428 | 0.8355011 | 0.954371 | 1.0404313 | 1.051549 |
| NADP-dependent isocitrate dehydrogenase, | 0.9751434 | 0.9662603 | 0.8844669 | 0.9053764 | 0.8976849 | 0.8206554 |
| cytosolic | | | | | | |
| Phase-1 RCT-57 | 0.8971916 | 1.1469693 | 0.9682211 | 0.9070805 | 0.9379908 | 0.9906325 |
| Phase-1 RCT-36 | 1.030901 | 1.2475444 | 1.1799008 | 1.0977485 | 0.9714263 | 1.0200162 |
| Apolipoprotein All | 0.5435066 | 1.5678413 | 1.987918 | 1.5839444 | 0.9862868 | 1.0646089 |
| Phase-1 RCT-68 | 1.1530347 | 0.9796814 | 1.0529821 | 1.0203646 | 1.0065945 | 1.0266638 |
| Multidrug resistant protein-2 | 7.092227 | 1.1528152 | 0.9448585 | 0.9741976 | 1.2755394 | 1.1055968 |
| Phase-1 RCT-39 | 1.0245346 | 1.2952933 | 1.0360687 | 1.1460522 | 1.0200223 | 0.9283769 |
| NIPK | 1.0552114 | 0.9519741 | 1.0666246 | 0.9562246 | 1.1841632 | 1.1066053 |
| F1-ATPase beta subunit | 0.79371 | 0.5824824 | 0.8080747 | 0.8267955 | 0.8315023 | 0.9552596 |
| Elongation factor-1 alpha | 1.1176045 | 0.6484225 | 0.6845446 | 0.8015745 | 0.77161 | 0.8027691 |
| Phase-1 RCT-24 | 0.7967128 | 1.1363827 | 1.0947534 | 1.1282943 | 1.4797441 | 1.2426472 |
| Beta-tubulin, class I | 0.6583831 | 0.9871375 | 1.1816227 | 0.9654858 | 1.4334968 | 1.2262316 |
| 14-3-3 zeta | 0.9066348 | 1.2662808 | 1.2563125 | 1.1771941 | 1.2368373 | 1.226684 |
| Alpha-prothymosin | 0.7361132 | 0.6973529 | 0.80111 | 1.000351 | 0.8341448 | 0.9432956 |
| Phase-1 RCT-58 | 0.9068411 | 1.3218652 | 0.7319452 | 0.8496094 | 0.9126416 | 0.981568 |
| Phase-1 RCT-290 | 1.1415051 | 1.2014552 | 1.5136594 | 0.9350773 | 1.2102562 | 1.556267 |
| Transitional endoplasmic reticulum ATPase | 0.942205 | 0.9040257 | 0.7998399 | 0.8562358 | 0.980682 | 0.9558682 |
| Beta-actin | 0.8088791 | 0.8696831 | 0.8721395 | 0.920567 | 1.0866628 | 1.0317171 |
| Thioredoxin-1 (Trx1) | 0.9178764 | 1.0573362 | 1.1275827 | 1.116077 | 0.96548 | 0.9753659 |
| Phase-1 RCT-174 | 1.0119662 | 1.0676042 | 0.8893547 | 0.9063985 | 0.8918485 | 1.0085996 |
| Beta-actin, sequence 2 | 0.4662434 | 1.0493075 | 1.0808219 | 1.0205427 | 1.1984866 | 1.0989606 |
| Phase-1 RCT-109 | 0.8002186 | 1.2139648 | 1.2598127 | 1.2234077 | 1.1899779 | 1.1954125 |
| Phase-1 RCT-154 | 0.8965516 | 1.7016653 | 0.980823 | 1.0320556 | 1.0729707 | 1.0544393 |
| Hypoxanthine-guanine | 0.9169424 | 1.1343545 | 1.1225835 | 1.0134984 | 1.1198276 | 1.0930597 |
| phosphoribosyltransferase | | | | | | |
| Phase-1 RCT-213 | 0.9363415 | 1.2205992 | 0.9979483 | 0.9530891 | 1.0905441 | 0.9883806 |
| Phase-1 RCT-260 | 0.9439116 | 1.4119507 | 0.9232401 | 1.1074986 | 1.0297005 | 1.0617855 |
| Phase-1 RCT-12 | 1.0265204 | 1.0798181 | 1.1643517 | 1.0332371 | 1.2407523 | 1.1556875 |
| Phase-1 RCT-293 | 0.9875286 | 1.0815189 | 1.0137541 | 1.1558373 | 1.0307124 | 0.988685 |
| Ribosomal protein S8 | 1.0653687 | 0.7431343 | 0.8651135 | 0.9604044 | 0.8443019 | 0.8887519 |
| Pyruvate kinase, muscle | 1.0484035 | 1.2932757 | 1.2452881 | 1.2199684 | 1.1862054 | 1.0725667 |
| Nucleoside diphosphate kinase beta isoform | 0.9163886 | 0.884272 | 0.9776926 | 1.0866206 | 0.9377444 | 0.9876292 |
| p55CDC | 0.9337932 | 0.6865053 | 1.051605 | 1.1683073 | 1.1957258 | 1.1232985 |
| Phase-1 RCT-156 | 0.890612 | 0.7806898 | 0.968241 | 0.9010282 | 0.8866788 | 0.7981598 |
| Ribosomal protein L13A | 0.7503555 | 1.3866626 | 1.2264642 | 1.254413 | 1.2894015 | 1.2024813 |
| Phase-1 RCT-258 | 0.7158876 | 1.0860363 | 0.9140077 | 0.9974074 | 1.0564446 | 0.9905501 |
| Insulin-like growth factor I | 0.7579089 | 0.709424 | 0.9254466 | 0.8495425 | 0.9629328 | 0.8727532 |
| Cytochrome P450 3A1 | 0.8437393 | 0.5189273 | 0.8659403 | 1.519406 | 1.1221949 | 1.3782157 |
| {Ribosomal protein 16} | 1.0189527 | 0.9292013 | 1.005176 | 1.0348061 | 0.9341015 | 0.9262213 |
| Organic cation transporter 3 | 1.1000855 | 1.2534969 | 1.0935893 | 1.0776142 | 0.9963742 | 0.9381064 |
| Calpain 2 | 1.0218024 | 0.9829903 | 0.945152 | 1.013435 | 1 | 0.9742946 |
| Phase-1 RCT-102 | 0.4721198 | 1.2196081 | 0.8758773 | 0.8047291 | 1.3712068 | 1.3858229 |
| Equilbrative nitrobenzylthioinosine-sensitive | 0.7580545 | 0.9166246 | 0.9875444 | 1.1539688 | 0.9181049 | 1.0572339 |
| nucleoside transporter | | | | | | |
| Phase-1 RCT-227 | 0.8302748 | 0.6260063 | 0.6572302 | 0.8640105 | 0.6245602 | 0.8048629 |
| N-hydroxy-2-acetylaminofluorene sulfotransferase (ST1C1) | 0.4771281 | 0.7114425 | 0.9279153 | 1.2160424 | 0.8657607 | 1.1063266 |
| Cathepsin S | 1.1595737 | 0.7847089 | 0.8652605 | 1.165069 | 1.3177131 | 1.048874 |
| Apolipoprotein CIII | 1.1004078 | 1.5180202 | 1.3591791 | 1.1904477 | 0.998946 | 1.2352605 |
| Cytochrome P450 2A3 | 1.0509179 | 0.665173 | 0.8606784 | 0.7682554 | 0.9893776 | 1.0264198 |
| Cytochrome P450 2C23 | 1.3112999 | 0.7230948 | 0.7749696 | 0.6826123 | 0.6833655 | 0.8507431 |
| Apolipoprotein C1 | 1.107757 | 0.6201949 | 0.7919222 | 0.8823807 | 0.6602634 | 0.7912113 |
| Betaine homocysteine methyltransferase (BHMT) | 1.2810035 | 1.129746 | 1.5316123 | 0.9437009 | 1.2850422 | 1.7162597 |
| Paraoxonase 1 | 0.9548219 | 0.5735727 | 0.7498099 | 0.8411365 | 0.7353622 | 0.6903971 |
| Phase-1 RCT-207 | 1.4879091 | 1.3872777 | 1.0244389 | 1.0684944 | 1.2428136 | 1.1899596 |
| Cathepsin B | 1.143755 | 0.821876 | 0.8303102 | 0.9787697 | 0.8102186 | 0.9576992 |
| Phase-1 RCT-144 | 0.9921536 | 1.0877491 | 0.9817022 | 0.9491012 | 1.048176 | 1.02095 |
| Alpha-1 microglobulin/bikunin precursor (Ambp) | 0.9681382 | 0.6530989 | 0.7669957 | 0.8109956 | 0.6906587 | 0.8181381 |
| Cyclin D1 | 0.5690771 | 0.7405773 | 1.0566667 | 1.0646968 | 1.4941002 | 1.0021977 |
| Presenilin-1 | 0.5327652 | 0.3854422 | 0.7789034 | 0.821778 | 0.7977765 | 0.8463997 |
| Protein tyrosine phosphatase, receptor type, D | 1.0210359 | 0.6368359 | 0.7267856 | 0.6862176 | 0.7377726 | 0.6965625 |
| Multidrug resistant protein-1 | 12.259249 | 1.1739591 | 1.0477154 | 1.0138515 | 1.2185552 | 1.1261876 |
| Cathepsin L, sequence 2 | 1.9855471 | 0.8275333 | 0.8361416 | 1.007718 | 0.8865035 | 0.8519818 |
| JNK1 stress activated protein kinase | 1.1870356 | 0.7572896 | 0.9421501 | 0.9863551 | 0.8081523 | 1.0172044 |
| Phase-1 RCT-43 | 0.8174197 | 1.2282279 | 0.9827249 | 0.9028301 | 0.9440594 | 0.9200205 |
| Tissue plasminogen activator | 0.9047335 | 0.9386561 | 0.7707032 | 0.8564374 | 0.9877828 | 0.9682371 |
| Ubiquitin conjugating enzyme (RAD 6 | 0.9620779 | 1.00141 | 0.9635546 | 1.026611 | 0.9985704 | 0.9760462 |
| homologue) | | | | | | |
| Phase-1 RCT-173 | 0.7949672 | 1.3240677 | 1.031273 | 1.2527386 | 1.2271923 | 1.5148808 |
| Ornithine decarboxylase | 1.2351828 | 1.15073 | 1.2374029 | 1.1110508 | 1.1016673 | 1.2372446 |
| Zinc finger protein | 0.8459533 | 1.0587054 | 0.8424792 | 0.9429043 | 1.0235914 | 0.9309002 |
| CDK108 | 0.8754491 | 0.9559699 | 0.7647808 | 1.0417413 | 1.0320579 | 1.1897756 |
| Phase-1 RCT-117 | 1.1238887 | 1.032555 | 1.0889145 | 1.2259506 | 1.4009981 | 1.1803563 |
| {Phase-1 RCT 98} | 1.0484035 | 0.8236564 | 0.9333033 | 0.7495763 | 0.8982525 | 0.9223698 |
| Aquaporin-3 (AQP3) | 0.9962442 | 1.2083824 | 1.0216213 | 0.9562349 | 0.9864455 | 0.9686655 |
| Cholesterol esterase | 0.678498 | 0.4822761 | 0.6470948 | 0.8342601 | 0.9227808 | 0.9537233 |
| Ribosomal protein S17 | 1.0431285 | 0.685667 | 0.748722 | 0.8671934 | 0.72199 | 0.7945023 |
| Poly(ADP-ribose) polymerase | 1.0871538 | 1.276317 | 1.0518146 | 1.0717946 | 1.0127122 | 1.1415846 |
| Annexin V | 0.8978122 | 0.7842762 | 1.0576888 | 0.9155309 | 1.0552477 | 0.9798816 |
| Phase-1 RCT-33 | 1.1182579 | 1.2744461 | 1.1710119 | 1.1115637 | 0.959688 | 0.9972343 |
| Phase-1 RCT-61 | 0.7307056 | 1.8413651 | 0.8607473 | 0.9390233 | 1.1533614 | 1.0625662 |
| Retinol dehydrogenase type III | 1.4052343 | 0.824669 | 1.0094551 | 1.0749313 | 0.8182661 | 0.855442 |
| ATPase inhibitor (rat mitochondrial IF1 protein) | 0.8897038 | 0.7027428 | 0.7923535 | 0.8057063 | 0.7242656 | 0.8436141 |
| Thymidylate synthase | 1.4269938 | 1.1429355 | 1.0072016 | 1.063028 | 1.137898 | 1.1587468 |
| Interleukin-18 | 1.1081985 | 1.4590118 | 1.0065275 | 1.184717 | 1.1894325 | 1.1318567 |
| Lecithin: cholesterol acyltransferase | 1.501615 | 0.9860977 | 1.0310174 | 1.0167733 | 1.069258 | 0.9517972 |
| Contrapsin-like protease inhibitor (CPi-21) | 0.4651098 | 0.8877485 | 0.6768019 | 0.7469792 | 1.1466548 | 0.8854579 |
| Proliferating cell nuclear antigen gene | 1.0882758 | 0.9720637 | 0.9666366 | 0.9698149 | 1.2146982 | 1.0792899 |
| Phase-1 RCT-230 | 0.7969879 | 1.3620398 | 1.0917063 | 1.0846314 | 1.1029825 | 1.1342537 |
| Cytochrome P450 2D18 | 1.0682192 | 0.8608636 | 0.9259492 | 0.866269 | 0.808384 | 0.9133211 |
| Phase-1 RCT-48 | 0.6532077 | 0.7948495 | 1.1858199 | 0.9226449 | 1.0791743 | 1.1780403 |
| Phase-1 RCT-292 | 1.0375394 | 1 | 0.9488284 | 0.9427624 | 0.9088342 | 0.9553739 |
| Arginosuccinate synthetase 1 | 1.2036899 | 0.900635 | 1.1229535 | 0.9097535 | 0.849264 | 1.10759 |
| C-reactive protein | 0.9948837 | 0.6672802 | 0.7259065 | 0.7354786 | 0.7961632 | 0.7977885 |

| | CLO | CLOZ | CLOZ | CLOZ | CLOZ | CLOZ | CLOZ |
|---|---|---|---|---|---|---|---|
| Compound-Dose | 250 | 45 | 45 | 45 | 180 | 180 | 180 |
| Animal Number | 1849 | 2427 | 2428 | 2429 | 2437 | 2438 | 2439 |
| Liver Toxicity Necrosis Classification | no | no | no | no | no | no | no |
| Gene Name | | | | | | | |
| Osteoactivin | 0.942643 | 0.9345597 | 1.0782278 | 1.168362 | 1.0470521 | 1.1242033 | 0.9710334 |
| Stathmin | 1.1342403 | 0.8232292 | 0.8721635 | 0.8904902 | 0.8432205 | 0.8283637 | 0.9235503 |
| Calpactin I heavy chain | 1.1056453 | 1.0424662 | 1.0292034 | 1.0741041 | 1.0335804 | 1.1515646 | 1.0861706 |
| Phase-1 RCT-179 | 0.9182175 | 0.8469471 | 0.907483 | 0.8085168 | 0.8478805 | 0.8052818 | 0.8416673 |
| Phase-1 RCT-107 | 0.8686106 | 1.0481966 | 1.1168023 | 0.9649762 | 0.9462215 | 1.149151 | 1.0776573 |
| Heme oxygenase | 0.8591921 | 1.3140186 | 1.0582203 | 1.6809986 | 1.212103 | 1.178355 | 0.997801 |
| Collagen type II | 1.4810424 | 0.8277276 | 0.8608191 | 0.9539287 | 0.9795343 | 0.9928786 | 1.1111845 |
| Phase-1 RCT-81 | 0.8196836 | 1.0518415 | 0.933387 | 1.0306301 | 1.0788796 | 1.2808901 | 0.9992294 |
| Glycine methyltransferase | 0.8128147 | 1.1744595 | 1.2875259 | 1.0426061 | 0.8554119 | 1.212035 | 1.1284148 |
| Cystatin C | 0.8513537 | 1.380179 | 1.0837369 | 0.8811646 | 1.0186778 | 1.1224916 | 1.1194959 |
| Melanoma-associated antigen ME491 | 0.9412081 | 0.8294854 | 0.9276912 | 1.6788665 | 0.9235169 | 0.9895932 | 0.8727967 |
| Phase-1 RCT-146 | 1.0771872 | 0.851919 | 0.907967 | 0.958549 | 0.9650508 | 0.9013935 | 0.9678264 |
| Gamma-actin, cytoplasmic | 0.6925843 | 1.0573552 | 0.7921193 | 0.9282278 | 0.8029016 | 0.8263077 | 0.695773 |
| Adenine nucleotide translocator 1 | 0.9393988 | 0.7190591 | 0.7845273 | 1.1559027 | 0.8429036 | 0.8258902 | 0.8009629 |
| Thymosin beta-10 | 1.071186 | 0.9140514 | 0.9753304 | 1.017989 | 0.9489488 | 1.0114425 | 0.9842529 |
| Insulin-like growth factor I, exon 6 | 1.4090173 | 0.9523994 | 1.0844959 | 1.5016441 | 0.9435549 | 0.9669859 | 0.8064049 |
| Phase-1 RCT-78 | 0.809614 | 1.0708239 | 1.0255597 | 1.0231851 | 1.1954168 | 1.0483286 | 1.0379058 |
| Alpha-2-microglobulin | 1.1094667 | 1.3477685 | 1.0173719 | 0.4448824 | 0.636858 | 0.5702727 | 1.0688483 |
| High affinity IgE receptor gamma chain | 0.9104615 | 1.1244863 | 1.070665 | 0.9305428 | 0.9970236 | 1.0981668 | 0.9284319 |
| (FcERIgamma) | | | | | | | |
| 60S ribosomal protein L6 | 0.8597454 | 0.8183888 | 0.8841578 | 0.9023686 | 0.883939 | 0.9862794 | 0.8509998 |
| IgE binding protein | 1.0087712 | 1.2024622 | 1.1438608 | 1.5231959 | 1.0140986 | 1.3287609 | 1.1711911 |
| Phase-1 RCT-149 | 1.1067505 | 0.9315257 | 0.9755478 | 1.0579466 | 1.0932525 | 1.3795416 | 1.0135627 |
| Cytochrome P450 2C11 | 1.1206931 | 1.3018594 | 0.9585214 | 0.9814552 | 0.8808082 | 1.1099949 | 1.2041823 |
| Uncoupling protein 2 | 1.2152932 | 0.9616864 | 1.0083333 | 1.1621724 | 0.9414444 | 1.0299327 | 0.9485458 |
| Cofilin | 0.8533951 | 0.8751642 | 0.9295514 | 0.8963463 | 0.9012654 | 0.9617379 | 0.8225982 |
| Alpha-tubulin | 1.0446901 | 0.889751 | 0.9065345 | 0.7655936 | 0.8253847 | 0.8622235 | 0.818026 |
| Stearyl-CoA desaturase, liver | 0.5725889 | 2.2190535 | 2.052268 | 0.7928913 | 1.6927136 | 1.7187047 | 2.110249 |
| Glutathione S-transferase theta-1 | 0.8401468 | 0.978968 | 1.0262979 | 0.8789942 | 0.9676088 | 1.0359911 | 0.913502 |
| alpha-1,2-fucosyltrans-ferase | 1.2444804 | 1.1404507 | 1.3460823 | 1.234495 | 1.2963603 | 1.1903796 | 1.4372929 |
| Phase-1 RCT-138 | 0.8745452 | 1.0485007 | 1.0358986 | 1.1104476 | 1.0327804 | 1.0940216 | 0.9785694 |
| Phase-1 RCT-295 | 1.0182475 | 1.0704443 | 1.0278729 | 1.2044538 | 0.9733306 | 1.1358646 | 0.957106 |
| Phase-1 RCT-164 | 0.9711573 | 1.0004438 | 0.8779839 | 0.8515667 | 0.8965274 | 0.9174798 | 0.8840923 |
| Alpha-2-macroglobulin, sequence 2 | 0.9040657 | 1.2175817 | 1.163226 | 1.2342695 | 1.1465824 | 1.2038592 | 1.3058445 |
| Biliverdin reductase | 1.18346 | 1.008118 | 0.9469398 | 0.9911757 | 0.857626 | 0.988672 | 0.8304783 |
| Phase-1 RCT-121 | 1.1120313 | 0.9014308 | 0.8794441 | 0.9311649 | 0.8848279 | 0.8327172 | 0.7945927 |
| Dynamin-1 (D100) | 0.8949668 | 1.2312182 | 1.1141444 | 1.0117599 | 1.1697279 | 1.0276473 | 1.2025849 |
| Phase-1 RCT-192 | 0.8240222 | 0.9663864 | 1.0092735 | 1.3018557 | 0.9193401 | 1.0517389 | 1.0652984 |
| Fatty acid synthase | 0.5580244 | 1.3990666 | 1.0928726 | 0.6334624 | 1.5900842 | 1.5051484 | 1.3524806 |
| Phase-1 RCT-211 | 1.1048447 | 1.0216249 | 1.0071342 | 1.3007137 | 1.0031493 | 1.1367681 | 1.049017 |
| Ribosomal protein S9 | 0.8848304 | 0.7682277 | 0.9390423 | 0.9993905 | 0.8167642 | 1.079867 | 0.8968208 |
| NADP-dependent isocitrate dehydrogenase, | 0.8452731 | 0.8197544 | 0.9239048 | 1.0763936 | 0.9535837 | 0.8552055 | 0.8077273 |
| cytosolic | | | | | | | |
| Phase-1 RCT-57 | 1.0513443 | 0.870555 | 0.7734267 | 0.8478402 | 0.8586693 | 0.865525 | 0.7655206 |
| Phase-1 RCT-36 | 1.0115303 | 0.9271184 | 0.9749132 | 0.9769129 | 1.107173 | 0.957581 | 1.0611248 |
| Apolipoprotein All | 1.1456043 | 1.2525221 | 0.8341496 | 1.0258114 | 1.0280713 | 1.7778276 | 1.1358485 |
| Phase-1 RCT-68 | 0.9824663 | 1.0027404 | 0.9281353 | 0.9344307 | 1.0251151 | 1.0465014 | 0.9752446 |
| Multidrug resistant protein-2 | 1.3751001 | 0.6773307 | 0.856035 | 0.9304552 | 0.7568665 | 0.8323587 | 0.88363 |
| Phase-1 RCT-39 | 1.1247054 | 1.0507189 | 0.9904726 | 1.0445082 | 1.0072001 | 1.0959737 | 1.049271 |
| NIPK | 1.1751956 | 1.0480307 | 1.0671515 | 1.062827 | 1.0193776 | 1.0201286 | 0.987518 |
| F1-ATPase beta subunit | 0.860224 | 1.1487073 | 1.0159132 | 0.9750627 | 1.0550187 | 1.0793104 | 0.9894683 |
| Elongation factor-1 alpha | 0.8435195 | 0.9204183 | 0.945982 | 0.92886 | 0.8789715 | 0.8481195 | 0.7768153 |
| Phase-1 RCT-24 | 1.137291 | 0.9935914 | 1.0015016 | 0.8978265 | 0.9457842 | 0.9283865 | 0.9967684 |
| Beta-tubulin, class I | 1.0499293 | 1.2325191 | 1.2749845 | 1.1082929 | 1.1240833 | 1.0384737 | 0.8635567 |
| 14-3-3 zeta | 1.4380044 | 0.9167296 | 0.8579238 | 0.8495657 | 0.8049418 | 0.8432838 | 0.7632357 |
| Alpha-prothymosin | 0.8847184 | 1.0214623 | 1.1052212 | 0.87868 | 0.8783554 | 0.9818801 | 0.8613361 |
| Phase-1 RCT-58 | 0.8444083 | 0.6045102 | 0.953896 | 1.3248037 | 0.7666957 | 0.9067524 | 0.4473656 |
| Phase-1 RCT-290 | 1.1688259 | 1.2627106 | 1.4786121 | 1.1982678 | 1.5022181 | 1.3311437 | 1.6472455 |
| Transitional endoplasmic reticulum ATPase | 1.1286395 | 0.9057104 | 0.9861389 | 0.7542841 | 0.8593006 | 0.9198292 | 0.7603732 |
| Beta-actin | 1.0713162 | 0.7843592 | 0.7819946 | 0.6980713 | 0.7010646 | 0.8523393 | 0.6061528 |
| Thioredoxin-1 (Trx1) | 0.8908579 | 1.3379657 | 1.1337161 | 1.2200658 | 1.03099 | 1.0583211 | 1.2064846 |
| Phase-1 RCT-174 | 0.9667956 | 0.9297754 | 1.009597 | 0.8652784 | 0.9484521 | 0.9476061 | 1.0928543 |
| Beta-actin, sequence 2 | 1.0445635 | 0.9368587 | 0.9820979 | 1.0093621 | 0.8409578 | 0.9703475 | 0.812731 |
| Phase-1 RCT-109 | 1.2552323 | 1.0066521 | 1.0455092 | 1.1691853 | 1.0093459 | 1.1760856 | 1.2301499 |
| Phase-1 RCT-154 | 1.5428135 | 0.9010297 | 0.8680368 | 0.9569144 | 0.8797327 | 0.9004101 | 0.9267489 |
| Hypoxanthine-guanine | 0.9887353 | 0.9609381 | 0.9592654 | 0.8918157 | 0.9240628 | 0.9057809 | 0.9645646 |
| phosphoribosyltransferase | | | | | | | |
| Phase-1 RCT-213 | 0.9689691 | 0.8728406 | 0.9892727 | 0.8355832 | 0.8905777 | 0.9453468 | 0.9125257 |
| Phase-1 RCT-260 | 1.0213096 | 1.0197037 | 0.9518663 | 1.0131873 | 1.0153747 | 1.1212322 | 1.0150019 |
| Phase-1 RCT-12 | 1.091345 | 1.001728 | 1.0018955 | 1.1101578 | 1.0449466 | 1.0380212 | 1.0296527 |
| Phase-1 RCT-293 | 0.9035178 | 1.1403168 | 1.0644103 | 1.1480901 | 1.2325428 | 1.21768 | 1.1672828 |
| Ribosomal protein S8 | 0.8039578 | 1.0604315 | 1.024273 | 1.0801711 | 1.0054064 | 0.9087281 | 1.0792147 |
| Pyruvate kinase, muscle | 1.1761512 | 0.9510486 | 0.8413215 | 1.1242154 | 0.9232183 | 1.0255103 | 0.9978729 |
| Nucleoside diphosphate kinase beta isoform | 1.0326612 | 0.9978365 | 1.0319128 | 1.0883455 | 1.1055046 | 1.2404032 | 1.0609589 |
| p55CDC | 1.0938914 | 0.862381 | 0.8985666 | 0.9518141 | 0.7676137 | 0.8761545 | 1.021652 |
| Phase-1 RCT-156 | 1.0449444 | 0.9542208 | 0.8821846 | 0.9307423 | 0.9823205 | 0.992625 | 0.8158907 |
| Ribosomal protein L13A | 1.2459906 | 0.9600459 | 1.0376356 | 1.1930791 | 1.0101775 | 1.0959662 | 1.0986358 |
| Phase-1 RCT-258 | 1.0349445 | 0.8421285 | 1.0137233 | 0.9774554 | 0.8604779 | 0.9996085 | 0.8876647 |
| Insulin-like growth factor I | 0.9441771 | 1.0052006 | 1.0291662 | 1.2078078 | 0.8766206 | 0.8865143 | 0.7816437 |
| Cytochrome P450 3A1 | 1.6833273 | 1.084458 | 0.9097803 | 0.7754917 | 0.6978479 | 0.5993384 | 1.0885098 |
| {Ribosomal protein 16} | 0.8602765 | 1.1238925 | 1.1153653 | 1.076916 | 1.0744066 | 1.0874766 | 1.1301306 |
| Organic cation transporter 3 | 0.8824397 | 0.8461368 | 0.9043416 | 0.8233886 | 0.9030331 | 0.8646184 | 0.8776564 |
| Calpain 2 | 1.0173839 | 0.8874924 | 0.9616121 | 1.0530019 | 0.9356594 | 0.9630903 | 0.9845938 |
| Phase-1 RCT-102 | 1.1545914 | 0.8562846 | 0.9860466 | 0.853003 | 0.6384113 | 0.6860642 | 0.5705361 |
| Equilbrative nitrobenzylthioinosine-sensitive | 1.0141022 | 1.1095665 | 0.943151 | 0.9182163 | 0.9162298 | 1.1084393 | 0.84232 |
| nucleoside transporter | | | | | | | |
| Phase-1 RCT-227 | 0.7370678 | 1.7778434 | 0.99026 | 0.994734 | 1.4124557 | 1.2002728 | 0.922794 |
| N-hydroxy-2-acetylaminofluorene sulfotransferase (ST1C1) | 1.0626724 | 1.1053389 | 0.9853115 | 0.8514848 | 0.9281993 | 0.9099991 | 0.8320577 |
| Cathepsin S | 1.236654 | 1.2678462 | 1.1386571 | 1.0732069 | 0.9096872 | 0.9115765 | 0.7995523 |
| Apolipoprotein CIII | 0.7963184 | 1.0141903 | 0.9164869 | 0.7260012 | 0.8667428 | 0.8842183 | 0.7832887 |
| Cytochrome P450 2A3 | 0.9155202 | 1.4032549 | 0.9727781 | 1.2020869 | 1.0968564 | 1.0539584 | 0.9970355 |
| Cytochrome P450 2C23 | 0.8478066 | 1.0262516 | 0.975217 | 1.146323 | 1.1468648 | 1.0763364 | 0.7897856 |
| Apolipoprotein C1 | 0.5750701 | 1.2010529 | 1.1213264 | 0.7563356 | 0.8998373 | 1.3660082 | 1.128233 |
| Betaine homocysteine methyltransferase (BHMT) | 1.2725301 | 1.9963094 | 1.7994177 | 1.3536216 | 1.7182473 | 1.7944863 | 1.946179 |
| Paraoxonase 1 | 0.7640103 | 0.9410453 | 1.0143371 | 0.9137919 | 0.9978377 | 0.9257082 | 0.8086424 |
| Phase-1 RCT-207 | 1.1118057 | 0.832097 | 1.1198542 | 0.8379142 | 0.8646782 | 0.9950517 | 1.0950443 |
| Cathepsin B | 0.8738011 | 1.0888652 | 1.0741798 | 1.0068811 | 1.1143695 | 1.0097767 | 1.069271 |
| Phase-1 RCT-144 | 1.03398 | 0.8709105 | 0.918478 | 0.800933 | 0.8436075 | 0.8310155 | 0.9012768 |
| Alpha-1 microglobulin/bikunin precursor (Ambp) | 0.7853707 | 1.0675732 | 0.9366026 | 0.9887813 | 1.08457 | 1.24618 | 0.9630785 |
| Cyclin D1 | 1.1624887 | 0.6729055 | 0.8840419 | 0.8954247 | 0.6192134 | 0.6860701 | 0.5582302 |
| Presenilin-1 | 0.9003035 | 1.0375314 | 0.9848902 | 1.3693697 | 1.1132132 | 1.1686412 | 0.9258992 |
| Protein tyrosine phosphatase, receptor type, D | 0.6918535 | 1.2425138 | 1.0054802 | 1.2116327 | 1.1251897 | 0.9600487 | 0.9470097 |
| Multidrug resistant protein-1 | 1.3986751 | 0.7609457 | 0.9558049 | 0.9530794 | 0.7813795 | 0.7809597 | 0.7335182 |
| Cathepsin L, sequence 2 | 1.2690121 | 0.7890319 | 0.7665939 | 1.110749 | 0.8774298 | 0.8270204 | 0.9004023 |
| JNK1 stress activated protein kinase | 0.8792153 | 1.5390176 | 1.4406097 | 0.9046421 | 1.089009 | 1.1040957 | 1.2033893 |
| Phase-1 RCT-43 | 1.0383524 | 0.7712193 | 0.7795381 | 0.8628752 | 1.0391754 | 0.8058645 | 0.7445085 |
| Tissue plasminogen activator | 1.0363616 | 1.0099232 | 1.0170149 | 1.028418 | 0.9573944 | 0.9035504 | 0.9759246 |
| Ubiquitin conjugating enzyme (RAD 6 | 0.9373851 | 0.8161327 | 0.921292 | 1.0369884 | 0.8535852 | 1.0132788 | 0.908407 |
| homologue) | | | | | | | |
| Phase-1 RCT-173 | 1.310106 | 0.8001115 | 0.9233553 | 1.127942 | 0.78852 | 0.8587228 | 1.2032715 |
| Ornithine decarboxylase | 1.490565 | 0.9862671 | 0.9391763 | 0.9184066 | 0.900362 | 0.939783 | 0.8897621 |
| Zinc finger protein | 0.9537275 | 0.7956351 | 0.941107 | 0.9406952 | 1.0067227 | 0.8663756 | 0.9179043 |
| CDK108 | 0.9889569 | 1.0606788 | 1.0925049 | 0.7449911 | 0.9446747 | 0.8606912 | 0.9875216 |
| Phase-1 RCT-117 | 1.5620015 | 0.9791164 | 0.9561293 | 1.1319755 | 1.1979011 | 1.2137002 | 1.1456724 |
| {Phase-1 RCT 98} | 1 | 1.3127183 | 1.0088943 | 1.3378121 | 1.0460153 | 0.9725323 | 1.0189484 |
| Aquaporin-3 (AQP3) | 0.9199994 | 1.0281225 | 0.9805326 | 0.9566085 | 1.0117993 | 0.9222493 | 0.9803752 |
| Cholesterol esterase | 1.1613569 | 0.80945 | 0.8798002 | 0.5625244 | 0.7918417 | 0.5305907 | 0.6794686 |
| Ribosomal protein S17 | 0.7032917 | 1.0104731 | 0.9323593 | 1.0095004 | 0.9221901 | 1.0857216 | 0.9459616 |
| Poly(ADP-ribose) polymerase | 1.0406271 | 0.9228308 | 0.9331031 | 0.8862852 | 1.0317184 | 1.0168998 | 0.8603865 |
| Annexin V | 0.9139581 | 1.0081031 | 1.2588427 | 1.0384034 | 0.978928 | 0.9014699 | 0.9790916 |
| Phase-1 RCT-33 | 1.073124 | 1.0037721 | 1.0676316 | 1.2376653 | 1.1676068 | 1.0312929 | 1.1231512 |
| Phase-1 RCT-61 | 1.5671376 | 0.7848545 | 0.9341611 | 0.9441888 | 0.8370518 | 0.9097829 | 0.7101918 |
| Retinol dehydrogenase type III | 0.9214893 | 1.1729091 | 0.9117292 | 1.0625468 | 0.8806607 | 1.102696 | 0.9090591 |
| ATPase inhibitor (rat mitochondrial IF1 protein) | 0.6935291 | 0.9908615 | 0.9549384 | 1.0543938 | 0.8842461 | 0.8280381 | 0.9317204 |
| Thymidylate synthase | 1.0540583 | 1.0709906 | 0.996575 | 0.919839 | 0.9630978 | 0.9348081 | 1.0721132 |
| Interleukin-18 | 1.0816269 | 0.9844092 | 0.9454166 | 0.9376687 | 0.9557911 | 0.9757918 | 1.0157405 |
| Lecithin: cholesterol acyltransferase | 0.9233862 | 0.9699749 | 1.0571389 | 0.7794594 | 0.8300314 | 1.0457162 | 0.8074275 |
| Contrapsin-like protease inhibitor (CPi-21) | 1.0240518 | 0.824904 | 0.8274779 | 1.1587368 | 0.7659369 | 0.733661 | 0.7606226 |
| Proliferating cell nuclear antigen gene | 1.0222216 | 0.9025081 | 0.9362502 | 0.9030359 | 0.9380521 | 0.9371417 | 0.9124237 |
| Phase-1 RCT-230 | 1.1564319 | 1.0646818 | 0.979135 | 1.1513941 | 0.998303 | 1.0771129 | 1.0527806 |
| Cytochrome P450 2D18 | 1.0619929 | 0.9194487 | 0.8539489 | 0.7847695 | 0.9243927 | 0.9885009 | 0.8048045 |
| Phase-1 RCT-48 | 1.0653282 | 1.465551 | 1.3113487 | 1.1287862 | 1.1095861 | 1.2640314 | 0.8921931 |
| Phase-1 RCT-292 | 0.9850354 | 1.010801 | 1.1326685 | 1.1530366 | 1.0817136 | 1.0794357 | 0.9769609 |
| Arginosuccinate synthetase 1 | 0.7447112 | 1.9207675 | 1.6019393 | 1.3079243 | 1.3719753 | 2.073992 | 1.7955859 |
| C-reactive protein | 0.7730857 | 1.2024 | 1.10192 | 1.1728142 | 1.1563897 | 1.0247034 | 0.9918584 |

| | CMC | CMC | CMC | CPHOS | CPHOS | CPHOS |
|---|---|---|---|---|---|---|
| Compound-Dose | 30 | 30 | 30 | 25 | 25 | 25 |
| Animal Number | 157 | 158 | 159 | 2147 | 2148 | 2149 |
| Liver Toxicity Necrosis Classification | no | no | no | no | no | no |
| Gene Name | | | | | | |
| Osteoactivin | 1.2889309 | 1.0545086 | 1.3016441 | 0.9385828 | 1.0281457 | 1.0240222 |
| Stathmin | 1.138056 | 0.9754817 | 0.9794297 | 1.0233871 | 1.0455183 | 1.0344216 |
| Calpactin I heavy chain | 1.1056103 | 1.0210295 | 1.084275 | 1.0141394 | 1.0665535 | 0.9659106 |
| Phase-1 RCT-179 | 1.0118146 | 1.048845 | 1.0120175 | 1.0019364 | 1.182583 | 0.8731843 |
| Phase-1 RCT-107 | 0.9765928 | 1.0210359 | 0.9047337 | 1.0753106 | 1.1667845 | 1.1292918 |
| Heme oxygenase | 1.3180435 | 0.9512541 | 1.0570935 | 0.9202954 | 1.2597101 | 0.8801882 |
| Collagen type II | 0.6959587 | 0.7719536 | 0.9475815 | 0.7156243 | 1.1939757 | 0.7977963 |
| Phase-1 RCT-81 | 0.9592848 | 1.0236173 | 1.0924891 | 0.903623 | 0.9092125 | 0.8994995 |
| Glycine methyltransferase | 1.0499519 | 1.3136109 | 1.0460423 | 1.1938634 | 1.3246374 | 1.4996918 |
| Cystatin C | 1.2707493 | 1.2188628 | 1.7879616 | 0.9407314 | 1.0226791 | 0.9771981 |
| Melanoma-associated antigen ME491 | 0.9851688 | 0.9846898 | 1.0029311 | 0.9944565 | 1.0127674 | 0.8913714 |
| Phase-1 RCT-146 | 0.9741255 | 0.9739841 | 0.9556292 | 1.0318807 | 1.0243378 | 0.998758 |
| Gamma-actin, cytoplasmic | 1.4135207 | 1.2651992 | 1.1447126 | 0.8709148 | 0.9287542 | 0.971497 |
| Adenine nucleotide translocator 1 | 0.8508092 | 0.8114212 | 0.799343 | 0.9027349 | 1.0615385 | 1.1587278 |
| Thymosin beta-10 | 0.9593846 | 1.0070457 | 0.9062389 | 1.1145022 | 1.2015641 | 0.8726415 |
| Insulin-like growth factor I, exon 6 | 1.6908125 | 2.286622 | 1.7243525 | 0.8712907 | 0.9729604 | 0.9705328 |
| Phase-1 RCT-78 | 0.8780007 | 0.9706608 | 0.9448109 | 0.8998479 | 0.99756 | 0.9054032 |
| Alpha-2-microglobulin | 1.5135995 | 0.9594142 | 1.238251 | 0.8542937 | 1.006148 | 1.1268616 |
| High affinity IgE receptor gamma chain | 1.0997562 | 0.9764376 | 1.0102549 | 0.8836824 | 1.105533 | 0.9361463 |
| (FcERIgamma) | | | | | | |
| 60S ribosomal protein L6 | 0.9209818 | 0.8845927 | 0.8731644 | 0.9177923 | 1.0076622 | 0.9318472 |
| IgE binding protein | 1.3009084 | 0.9910353 | 1.098371 | 0.9150372 | 1.1095983 | 0.898968 |
| Phase-1 RCT-149 | 0.9890046 | 0.9709206 | 0.9045722 | 0.9979214 | 0.9630014 | 0.9441901 |
| Cytochrome P450 2C11 | 0.8436419 | 0.7445204 | 0.7910663 | 1.0046109 | 1.0602225 | 1.306577 |
| Uncoupling protein 2 | 1.213779 | 0.9315023 | 0.9946378 | 0.9759121 | 1.0233186 | 0.8813807 |
| Cofilin | 0.8364256 | 0.9502775 | 1.0385445 | 0.8624392 | 1.044969 | 0.9616403 |
| Alpha-tubulin | 1.1389464 | 0.8272827 | 0.5871934 | 1.1459824 | 1.0066224 | 1.328019 |
| Stearyl-CoA desaturase, liver | 0.9209686 | 2.2997887 | 0.8117178 | 0.8868261 | 1.1760027 | 0.4962333 |
| Glutathione S-transferase theta-1 | 0.6808211 | 0.8649103 | 0.7386033 | 1.3050172 | 1.0518357 | 1.1374291 |
| alpha-1,2-fucosyltrans-ferase | 1.2903057 | 1.3973113 | 1.1638727 | 1.0942904 | 1.3014586 | 0.7029221 |
| Phase-1 RCT-138 | 1.0235499 | 0.9277945 | 1.0145626 | 0.8300493 | 1.0324403 | 0.9088556 |
| Phase-1 RCT-295 | 1.1949276 | 1.1328909 | 1.1393987 | 1.1225487 | 1.2035258 | 1.0022452 |
| Phase-1 RCT-164 | 0.8560562 | 0.9055933 | 0.7644439 | 0.9083982 | 0.9036491 | 1.0173072 |
| Alpha-2-macroglobulin, sequence 2 | 1.3707069 | 1.3301984 | 1.6245681 | 0.9173669 | 0.8591226 | 1.0186356 |
| Biliverdin reductase | 0.9141613 | 0.7243791 | 0.7587318 | 1.0752407 | 1.081202 | 1.0037645 |
| Phase-1 RCT-121 | 0.931588 | 0.778236 | 0.8180615 | 1.0709227 | 1.0668179 | 0.9305875 |
| Dynamin-1 (D100) | 0.9570632 | 1.0458424 | 1.0423245 | 1.0454576 | 1.0893102 | 0.9337162 |
| Phase-1 RCT-192 | 1.1356978 | 1.0617926 | 0.9793063 | 0.9784923 | 1.0990236 | 1.0780498 |
| Fatty acid synthase | 0.4084545 | 0.9070243 | 0.4495739 | 1.4917884 | 0.71807 | 0.7325774 |
| Phase-1 RCT-211 | 1.4891303 | 1.2590413 | 1.013599 | 1.1128601 | 1.0726202 | 1.0272896 |
| Ribosomal protein S9 | 1.0048176 | 0.8802258 | 0.8158299 | 1.1216217 | 1.1694349 | 1.1624703 |
| NADP-dependent isocitrate dehydrogenase, | 0.9700021 | 1.0891757 | 1.1172715 | 0.8177962 | 1.0937171 | 1.0205768 |
| cytosolic | | | | | | |
| Phase-1 RCT-57 | 0.9441357 | 0.8338573 | 0.8308747 | 0.9605545 | 0.8807865 | 0.9999284 |
| Phase-1 RCT-36 | 1.0642142 | 1.1983793 | 0.8955938 | 0.8560463 | 0.9046674 | 0.8044834 |
| Apolipoprotein All | 1.0290152 | 1.2074348 | 0.9191806 | 1.1803539 | 1.0616817 | 0.9664375 |
| Phase-1 RCT-68 | 1.0766162 | 0.9868926 | 0.9865466 | 1.0561926 | 1.0736389 | 1.0156757 |
| Multidrug resistant protein-2 | 1.040785 | 0.8804286 | 0.9993833 | 1.1173137 | 0.9741488 | 1.100803 |
| Phase-1 RCT-39 | 1.2567 | 1.2196299 | 1.1933078 | 1.021753 | 1.1314522 | 0.8390747 |
| NIPK | 1.0381786 | 1.2986808 | 1.1326978 | 0.9683511 | 0.9900663 | 0.9833118 |
| F1-ATPase beta subunit | 1.1297944 | 1.2750486 | 1.3521137 | 0.9621369 | 0.8590091 | 1.0296813 |
| Elongation factor-1 alpha | 1.2237678 | 1.3061694 | 1.3322058 | 0.8598588 | 0.9516273 | 0.9377559 |
| Phase-1 RCT-24 | 1.2737138 | 1.0266135 | 0.7375141 | 1.2605088 | 1.075093 | 1.2692897 |
| Beta-tubulin, class I | 1.303133 | 1.0861418 | 0.7138693 | 1.2561872 | 1.1219414 | 1.22554 |
| 14-3-3 zeta | 1.0354699 | 0.8428963 | 0.7429472 | 1.1294906 | 1.1294801 | 1.0093846 |
| Alpha-prothymosin | 0.9378611 | 1.0330721 | 1.1462225 | 0.8252998 | 0.9835648 | 0.8037432 |
| Phase-1 RCT-58 | 1.3915466 | 0.7155982 | 0.9558391 | 1.3824621 | 1.3192601 | 0.720627 |
| Phase-1 RCT-290 | 1.2492093 | 1.2436066 | 1.3814807 | 1.171331 | 0.9258006 | 0.9913524 |
| Transitional endoplasmic reticulum ATPase | 0.6977691 | 0.7633063 | 0.740975 | 0.9006811 | 0.8733001 | 0.9998021 |
| Beta-actin | 1.1827754 | 0.8142929 | 0.721509 | 1.255621 | 0.7513372 | 1.1514144 |
| Thioredoxin-1 (Trx1) | 1.6019262 | 1.5259026 | 1.4931053 | 0.9464825 | 1.0390656 | 0.9021819 |
| Phase-1 RCT-174 | 0.9278058 | 1.0658637 | 0.9582974 | 0.9831263 | 1.0509114 | 1.0902169 |
| Beta-actin, sequence 2 | 1.1307719 | 0.9017224 | 0.8868564 | 1.0164118 | 0.990465 | 0.9264483 |
| Phase-1 RCT-109 | 1.2973969 | 1.2506782 | 1.0885146 | 1.163463 | 1.022953 | 0.9269893 |
| Phase-1 RCT-154 | 0.9158813 | 0.9100363 | 0.9377422 | 0.9750838 | 0.9837943 | 0.9949173 |
| Hypoxanthine-guanine | 0.8651931 | 1.0298307 | 0.9170283 | 0.9127287 | 0.9762687 | 1.0773463 |
| phosphoribosyltransferase | | | | | | |
| Phase-1 RCT-213 | 0.9369665 | 0.8941652 | 0.8575115 | 0.9256769 | 0.9512628 | 0.9784131 |
| Phase-1 RCT-260 | 1.2438421 | 1.1883491 | 1.2690058 | 1.023167 | 1.2436676 | 1.0395348 |
| Phase-1 RCT-12 | 1.1355 | 1.0437195 | 0.8413268 | 1.1929742 | 1.026609 | 1.0064231 |
| Phase-1 RCT-293 | 1.0088599 | 1.039677 | 0.9673254 | 0.8670381 | 1.0890228 | 0.9142185 |
| Ribosomal protein S8 | 1.432913 | 1.3762776 | 1.2855463 | 0.9867952 | 1.2390474 | 0.8825999 |
| Pyruvate kinase, muscle | 1.0677803 | 1.07436 | 1.0371344 | 1.0614388 | 1.0884738 | 0.8798138 |
| Nucleoside diphosphate kinase beta isoform | 0.860248 | 0.9533583 | 0.8102428 | 1.0120844 | 1.1983564 | 1.212573 |
| p55CDC | 1.2887709 | 0.8829361 | 0.7040952 | 1.0935701 | 1.0237706 | 1.0450071 |
| Phase-1 RCT-156 | 0.8693345 | 0.8841302 | 0.8032814 | 1.1430646 | 1.0538337 | 1.0902802 |
| Ribosomal protein L13A | 1.3920814 | 1.3130094 | 1.072649 | 1.2529446 | 1.1315937 | 0.9579014 |
| Phase-1 RCT-258 | 0.8473513 | 0.8703022 | 0.8473553 | 0.9707671 | 0.9860947 | 0.9974753 |
| Insulin-like growth factor I | 1.3889232 | 1.4390445 | 1.2932276 | 0.7286124 | 1.1460044 | 1.110507 |
| Cytochrome P450 3A1 | 1.6228986 | 0.6517804 | 1.5991155 | 1.3256538 | 1.2951434 | 1.1517206 |
| {Ribosomal protein 16} | 1.1364644 | 1.1561252 | 1.1706502 | 1.019827 | 1.0911468 | 0.8667322 |
| Organic cation transporter 3 | 0.8466633 | 0.8288421 | 0.9120985 | 0.9264104 | 0.9521025 | 0.95617 |
| Calpain 2 | 0.9971196 | 0.9743212 | 1.073631 | 0.9535494 | 1.011966 | 0.9449452 |
| Phase-1 RCT-102 | 1.3789635 | 1.2537564 | 1.0675772 | 0.6658932 | 0.7927386 | 0.9729164 |
| Equilbrative nitrobenzylthioinosine-sensitive | 1.5460588 | 1.612324 | 1.2344618 | 0.8544525 | 1.2272296 | 1.0384793 |
| nucleoside transporter | | | | | | |
| Phase-1 RCT-227 | 1.4171139 | 1.2272613 | 1.4298489 | 0.8707761 | 1.175167 | 0.9299356 |
| N-hydroxy-2-acetylaminofluorene sulfotransferase (ST1C1) | 1.8389548 | 1.6019073 | 1.7789648 | 0.8115046 | 1.2367915 | 1.0822898 |
| Cathepsin S | 0.8223536 | 0.6180137 | 0.7601706 | 0.9936855 | 1.0388556 | 1.0051111 |
| Apolipoprotein CIII | 0.7365875 | 0.8518068 | 0.9286248 | 0.866156 | 0.9071874 | 1.0153701 |
| Cytochrome P450 2A3 | 1.1941499 | 1.2349095 | 1.4814113 | 0.8538348 | 0.9926203 | 1.2989792 |
| Cytochrome P450 2C23 | 1.0035026 | 0.8447886 | 1.0660838 | 0.8097371 | 1.1567591 | 0.8830919 |
| Apolipoprotein C1 | 0.8862153 | 0.9384623 | 0.9418523 | 0.8977211 | 0.815743 | 0.9307741 |
| Betaine homocysteine methyltransferase (BHMT) | 1.3929266 | 1.4566138 | 1.6326904 | 1.1806445 | 0.8981399 | 0.959125 |
| Paraoxonase 1 | 1.0672224 | 1.4343208 | 1.6888217 | 0.8230721 | 0.9554172 | 1.1936886 |
| Phase-1 RCT-207 | 0.8967421 | 0.9482688 | 0.8547812 | 1.1363107 | 0.9121698 | 0.9783226 |
| Cathepsin B | 1.0475775 | 1.1001062 | 1.29186 | 0.7163477 | 0.9786389 | 0.8533286 |
| Phase-1 RCT-144 | 0.7581425 | 0.8790797 | 0.7973008 | 0.9885905 | 0.9190999 | 0.9982378 |
| Alpha-1 microglobulin/bikunin precursor (Ambp) | 0.9169998 | 1.025451 | 1.1161635 | 0.7550666 | 0.9001037 | 0.886622 |
| Cyclin D1 | 0.7159886 | 0.7777053 | 0.8142181 | 1.0794629 | 0.9045079 | 1.4580586 |
| Presenilin-1 | 0.8585627 | 0.9522178 | 1.3341583 | 0.7373524 | 0.7190445 | 0.9450786 |
| Protein tyrosine phosphatase, receptor type, D | 1.3501521 | 1.4853935 | 1.5429028 | 0.8469745 | 1.2397736 | 0.9041018 |
| Multidrug resistant protein-1 | 0.9020272 | 0.8103981 | 0.879083 | 1.0609937 | 0.9791863 | 1.1057808 |
| Cathepsin L, sequence 2 | 1.4328537 | 1.3549007 | 1.3874577 | 0.9572023 | 1.0576174 | 1.128434 |
| JNK1 stress activated protein kinase | 1.0180285 | 1.1635323 | 1.1550738 | 1.1547185 | 1.0531117 | 1.0829595 |
| Phase-1 RCT-43 | 0.9104521 | 0.890032 | 0.7361316 | 1.1114337 | 1.0175463 | 1.0066051 |
| Tissue plasminogen activator | 1.2411344 | 1.183396 | 1.1391244 | 0.8539276 | 0.9831994 | 0.820228 |
| Ubiquitin conjugating enzyme (RAD 6 | 1.0264703 | 0.9279189 | 0.8954997 | 1.0826395 | 1.070663 | 1.0792493 |
| homologue) | | | | | | |
| Phase-1 RCT-173 | 0.9348309 | 0.8314083 | 0.8492696 | 1.2367823 | 0.7943306 | 1.0552132 |
| Ornithine decarboxylase | 1.1282605 | 0.8181173 | 0.7826803 | 1.3094934 | 1.0750128 | 1.1413779 |
| Zinc finger protein | 1.0014509 | 0.9191709 | 1.0496218 | 0.8478718 | 1.0078776 | 1.0913663 |
| CDK108 | 1.2156453 | 0.9753763 | 1.0356503 | 1.0263367 | 1.0032209 | 1.1511014 |
| Phase-1 RCT-117 | 1.0663085 | 0.8979606 | 1.1097826 | 1.0824937 | 0.7908813 | 0.8764553 |
| {Phase-1 RCT 98} | 0.9272282 | 1.027321 | 1.0336976 | 0.956545 | 1.0640755 | 0.969441 |
| Aquaporin-3 (AQP3) | 0.9521 | 1.0134764 | 1.0130141 | 0.978933 | 0.9410775 | 1.0309263 |
| Cholesterol esterase | 0.8157204 | 0.7745518 | 0.7175704 | 1.0149586 | 0.9975607 | 1.477457 |
| Ribosomal protein S17 | 1.6335303 | 1.5597183 | 1.5429296 | 0.9615101 | 0.9514881 | 1.1196235 |
| Poly(ADP-ribose) polymerase | 0.7781844 | 0.8459557 | 0.7581823 | 1.0325987 | 0.951148 | 1.0048362 |
| Annexin V | 0.9261032 | 0.7993922 | 0.826671 | 0.902122 | 0.9733622 | 1.0707493 |
| Phase-1 RCT-33 | 1.1003577 | 1.3398391 | 0.8580387 | 0.7894226 | 0.8703879 | 0.7648079 |
| Phase-1 RCT-61 | 0.9778713 | 0.8018869 | 0.9305973 | 1.0540262 | 0.9951435 | 0.8698806 |
| Retinol dehydrogenase type III | 0.9159572 | 0.9964966 | 0.8528819 | 0.8671441 | 0.9571379 | 1.1037244 |
| ATPase inhibitor (rat mitochondrial IF1 protein) | 1.3941047 | 1.4625309 | 1.5045375 | 0.7282732 | 0.902312 | 1.0929203 |
| Thymidylate synthase | 0.9670084 | 0.7003186 | 0.7030637 | 0.981326 | 0.9658339 | 1.0690069 |
| Interleukin-18 | 0.9305471 | 0.7892528 | 0.8257437 | 0.9986783 | 0.9151627 | 1.0417435 |
| Lecithin: cholesterol acyltransferase | 1.0863485 | 1.1005692 | 1.0183388 | 0.8875993 | 1.1093799 | 1.036594 |
| Contrapsin-like protease inhibitor (CPi-21) | 1.6121846 | 1.475946 | 1.5521822 | 0.9278949 | 0.923085 | 0.9508694 |
| Proliferating cell nuclear antigen gene | 1.0346377 | 0.8162182 | 0.8265908 | 1.078667 | 0.9518063 | 0.9973624 |
| Phase-1 RCT-230 | 1.3307012 | 1.0715871 | 1.0526913 | 1.1171075 | 1.0580235 | 0.9127675 |
| Cytochrome P450 2D18 | 1.0616637 | 0.9235806 | 0.8977004 | 0.9671055 | 0.9959737 | 1.0467342 |
| Phase-1 RCT-48 | 0.9514334 | 1.0147374 | 0.8460403 | 0.8509972 | 1.0256742 | 1.0035248 |
| Phase-1 RCT-292 | 0.8362132 | 0.8390407 | 0.8824185 | 0.9232436 | 0.9848138 | 0.9543792 |
| Arginosuccinate synthetase 1 | 0.8477578 | 1.0763304 | 1.0284709 | 1.072223 | 0.9436058 | 0.8247016 |
| C-reactive protein | 1.1300908 | 1.0942255 | 1.1718067 | 0.904013 | 1.6750377 | 1.0280669 |

| | CPHOS | CPHOS | CPHOS | CYCA | CYCA | CYCA | CYCA |
|---|---|---|---|---|---|---|---|
| Compound-Dose | 100 | 100 | 100 | 20 | 20 | 20 | 80 |
| Animal Number | 2157 | 2158 | 2159 | 427 | 428 | 429 | 437 |
| Liver Toxicity Necrosis Classification | no | no | no | no | no | no | no |
| Gene Name | | | | | | | |
| Osteoactivin | 1.0436045 | 1.067628 | 1.102224 | 0.896871 | 0.864158 | 0.9913611 | 0.8853764 |
| Stathmin | 1.0119145 | 1.0053306 | 0.8767463 | 0.7232014 | 0.6512495 | 0.8213362 | 1.0253643 |
| Calpactin I heavy chain | 1.0187091 | 0.9155572 | 0.9156488 | 0.9420722 | 0.8040061 | 1.1129271 | 1.0056458 |
| Phase-1 RCT-179 | 0.983916 | 0.9271128 | 0.8622924 | 0.7956734 | 1.0344636 | 1.0024524 | 1.0044488 |
| Phase-1 RCT-107 | 1.048173 | 1.1764516 | 1.5059857 | 1.2788833 | 1.0549026 | 0.8805143 | 0.7745631 |
| Heme oxygenase | 1.1161497 | 1.3533763 | 1.5083226 | 1.0948458 | 1.2645437 | 1.0805423 | 1.1430535 |
| Collagen type II | 0.948456 | 0.883929 | 0.623841 | 0.9747517 | 1.1707809 | 1.2646807 | 1.267839 |
| Phase-1 RCT-81 | 0.9792534 | 0.908452 | 0.908176 | 1.0374745 | 0.9996448 | 1.0707949 | 0.8833119 |
| Glycine methyltransferase | 1.1597422 | 1.3797096 | 3.2440681 | 1.357226 | 1.5469682 | 0.9010144 | 0.7754315 |
| Cystatin C | 0.9711461 | 1.1065692 | 0.9417244 | 0.9601196 | 0.9849886 | 0.788917 | 0.8845341 |
| Melanoma-associated antigen ME491 | 0.8571029 | 0.8831961 | 0.9164518 | 0.8381214 | 0.7500755 | 1.069553 | 0.9174312 |
| Phase-1 RCT-146 | 0.8994604 | 1.0923296 | 0.9932351 | 0.7160226 | 0.583431 | 0.8087221 | 0.9935265 |
| Gamma-actin, cytoplasmic | 0.873888 | 0.9208146 | 0.6508007 | 0.9543847 | 1.0900416 | 0.9570841 | 1.1188185 |
| Adenine nucleotide translocator 1 | 0.9085509 | 0.7560513 | 1.1668199 | 0.8392341 | 1.1152223 | 0.9800035 | 1.0734262 |
| Thymosin beta-10 | 1.0258489 | 0.8657708 | 0.6551985 | 1.110147 | 1.4052982 | 1.0154701 | 1.2575134 |
| Insulin-like growth factor I, exon 6 | 1.0919908 | 1.5580198 | 0.7266054 | 1.350137 | 1.1882974 | 1.4938877 | 0.8907163 |
| Phase-1 RCT-78 | 0.9152142 | 0.9973647 | 0.8268464 | 1.144422 | 1.0361567 | 1.0926943 | 0.8570016 |
| Alpha-2-microglobulin | 0.9917681 | 0.2989539 | 1.0007262 | 1.0279642 | 1.394355 | 1.1970104 | 0.5680637 |
| High affinity IgE receptor gamma chain | 0.8734645 | 0.8896277 | 0.9763599 | 0.8659391 | 0.8138246 | 0.906005 | 1.0431533 |
| (FcERIgamma) | | | | | | | |
| 60S ribosomal protein L6 | 1.0145407 | 0.8728239 | 0.8314827 | 0.9593333 | 1.0840175 | 0.9726762 | 1.2178493 |
| IgE binding protein | 0.8796755 | 1.0254151 | 1.0188848 | 1.0249646 | 0.809337 | 0.9590459 | 1.0058296 |
| Phase-1 RCT-149 | 1.0732262 | 1.0863087 | 1.1993468 | 1.155819 | 0.9939852 | 1.3293812 | 1.0107745 |
| Cytochrome P450 2C11 | 0.7113605 | 0.4146271 | 1.0875299 | 0.9030185 | 0.6965876 | 0.9798612 | 0.9443598 |
| Uncoupling protein 2 | 0.8637664 | 1.0205239 | 0.7241208 | 0.7818076 | 1.0664377 | 1.0237813 | 1.2744315 |
| Cofilin | 1.0073184 | 0.8938242 | 0.9355767 | 1.0193903 | 1.0828006 | 0.9496704 | 1.143015 |
| Alpha-tubulin | 1.1977708 | 0.7655994 | 0.8188624 | 0.6907163 | 1.0392317 | 1.0518792 | 1.2151084 |
| Stearyl-CoA desaturase, liver | 0.3741523 | 0.1131924 | 0.0532749 | 2.0310347 | 2.3052778 | 2.7010753 | 3.4864995 |
| Glutathione S-transferase theta-1 | 1.063044 | 0.5751815 | 0.742129 | 1.0194448 | 1.2877849 | 1.3139528 | 1.1410908 |
| alpha-1,2-fucosyltrans-ferase | 1.2128539 | 0.748928 | 0.5304248 | 1.5073184 | 1.4961586 | 1.3676933 | 1.5866662 |
| Phase-1 RCT-138 | 0.917102 | 1.0895128 | 1.0611228 | 0.9874259 | 0.8910696 | 0.9243786 | 1.0114303 |
| Phase-1 RCT-295 | 1.1723812 | 0.8065118 | 0.8776364 | 1.172515 | 1.1763374 | 1.1192122 | 1.2327982 |
| Phase-1 RCT-164 | 1.4428737 | 0.8737696 | 0.9270603 | 0.7093401 | 0.8881891 | 0.9641743 | 1.1343135 |
| Alpha-2-macroglobulin, sequence 2 | 0.9484806 | 1.095003 | 1.1934747 | 1.1219518 | 1.0011367 | 0.8369481 | 0.9253401 |
| Biliverdin reductase | 0.881697 | 0.9203205 | 1.0000472 | 1.0003603 | 0.6620081 | 1.0336281 | 1.1076303 |
| Phase-1 RCT-121 | 1.0228134 | 1.2448784 | 0.9381557 | 0.6176245 | 0.7379192 | 0.9220102 | 1.0875999 |
| Dynamin-1 (D100) | 0.9805073 | 0.8392251 | 0.9018847 | 1.039085 | 0.970882 | 1.0305948 | 0.9062102 |
| Phase-1 RCT-192 | 1.0414662 | 1.0857983 | 1.1016004 | 1.0025299 | 1.0290422 | 0.9984779 | 1.0606666 |
| Fatty acid synthase | 0.814455 | 0.6404005 | 0.4287907 | 1.1571031 | 1.0082945 | 1.4467834 | 2.0115762 |
| Phase-1 RCT-211 | 1.216077 | 1.000599 | 0.9325882 | 1.3868315 | 0.9398359 | 1.3432729 | 1.0266143 |
| Ribosomal protein S9 | 1.018121 | 0.7541112 | 0.9990661 | 0.9814152 | 1.2308806 | 1.0536163 | 0.9790087 |
| NADP-dependent isocitrate dehydrogenase, | 1.0510317 | 0.8988023 | 1.0620543 | 1.0988337 | 1.0949202 | 1.0282528 | 1.1829741 |
| cytosolic | | | | | | | |
| Phase-1 RCT-57 | 1 | 0.9493192 | 1.0188458 | 0.6973543 | 0.9164693 | 0.9776433 | 0.9583539 |
| Phase-1 RCT-36 | 0.9782172 | 0.8638299 | 0.7841331 | 1.0367752 | 0.8182085 | 1.1903628 | 1.0321101 |
| Apolipoprotein All | 0.9780538 | 0.8881643 | 0.8840684 | 1.630234 | 1.2929926 | 1.4360539 | 1.2098233 |
| Phase-1 RCT-68 | 1.0344716 | 1.0300975 | 1.0448085 | 1.068036 | 0.8848832 | 1.0196689 | 0.8715596 |
| Multidrug resistant protein-2 | 1.023925 | 1.128672 | 1.3552914 | 1.1465071 | 1.1947309 | 1.0720819 | 1.0207369 |
| Phase-1 RCT-39 | 1.0074171 | 1.1235346 | 0.8162031 | 0.964907 | 0.9726074 | 1 | 1.0599512 |
| NIPK | 1.0490206 | 1.292463 | 0.8721133 | 0.9765518 | 0.8441107 | 1.1775259 | 0.9242439 |
| F1-ATPase beta subunit | 0.9746374 | 0.778728 | 0.8887776 | 1.2498817 | 1.5791855 | 0.9205267 | 1.0639833 |
| Elongation factor-1 alpha | 1.1355962 | 1.0605838 | 0.9263971 | 1.0585471 | 1.3617827 | 0.9093326 | 0.8378928 |
| Phase-1 RCT-24 | 1.2347034 | 0.8340559 | 0.892236 | 0.8441142 | 0.9002981 | 1.1313798 | 1.2823185 |
| Beta-tubulin, class I | 1.1814804 | 0.6538962 | 0.6879602 | 0.9271002 | 0.8924828 | 1.1768804 | 1.0840712 |
| 14-3-3 zeta | 1.2411304 | 0.823343 | 0.7916379 | 0.8631851 | 0.7935494 | 1.0484487 | 1.2461547 |
| Alpha-prothymosin | 1.0818585 | 0.645363 | 0.6203782 | 1.0820533 | 1.1567007 | 0.868437 | 1.1662757 |
| Phase-1 RCT-58 | 1.0347291 | 1.0703315 | 0.9563615 | 0.9675276 | 1.2806519 | 1.3103536 | 1.7793968 |
| Phase-1 RCT-290 | 0.6873617 | 0.7077835 | 0.8375409 | 1.4556903 | 1.5008878 | 1.4937136 | 1.1815686 |
| Transitional endoplasmic reticulum ATPase | 0.9095313 | 0.863413 | 0.9474051 | 0.7335654 | 0.858183 | 0.9798644 | 0.919417 |
| Beta-actin | 1.3811171 | 0.9341973 | 0.72676 | 0.8571941 | 1.0549438 | 0.8969235 | 0.9976649 |
| Thioredoxin-1 (Trx1) | 0.9618254 | 0.9127033 | 0.8598597 | 1.2005306 | 1.3852628 | 1.0249723 | 1.2742435 |
| Phase-1 RCT-174 | 0.9353867 | 0.9812252 | 1.0338815 | 1.0651221 | 0.9226402 | 1.0819923 | 1.0766548 |
| Beta-actin, sequence 2 | 1.009516 | 0.7994247 | 0.7978183 | 0.9061514 | 1.1042447 | 0.9861549 | 1.267696 |
| Phase-1 RCT-109 | 1.1131849 | 1.038586 | 0.707365 | 1.2158397 | 1.1518681 | 1.1532438 | 1.2002677 |
| Phase-1 RCT-154 | 1.0445738 | 0.8485645 | 0.9701576 | 0.6734484 | 0.5731989 | 1.0326418 | 1.0825689 |
| Hypoxanthine-guanine | 0.9193561 | 0.772777 | 0.8641161 | 1.0449746 | 1.3379214 | 1.2211837 | 1.2669691 |
| phosphoribosyltransferase | | | | | | | |
| Phase-1 RCT-213 | 1.0244302 | 0.9272488 | 0.8807998 | 0.6772608 | 0.8031379 | 0.9690344 | 1.0253936 |
| Phase-1 RCT-260 | 0.9269028 | 1.1136956 | 1.0292492 | 0.7607472 | 0.7471709 | 0.8392023 | 0.8231553 |
| Phase-1 RCT-12 | 1.1386274 | 1.0071388 | 1.0244678 | 0.9844428 | 0.8707415 | 1.1369476 | 1.0085766 |
| Phase-1 RCT-293 | 0.8912653 | 0.9368975 | 0.9429922 | 0.8194093 | 0.8116044 | 0.9999101 | 1.0475659 |
| Ribosomal protein S8 | 0.9950952 | 0.864613 | 0.9054608 | 1.1456726 | 1.3415077 | 1.0083742 | 0.96948 |
| Pyruvate kinase, muscle | 0.9707277 | 1.0191628 | 0.80258 | 1.0857997 | 0.9753692 | 1.2616954 | 0.8947152 |
| Nucleoside diphosphate kinase beta isoform | 0.9788076 | 0.8819379 | 0.9694283 | 1.1886274 | 1.2065467 | 1.1322056 | 1.2684265 |
| p55CDC | 0.9412105 | 1.0592881 | 0.9494747 | 1.052323 | 1.0092947 | 1.2670685 | 0.9050029 |
| Phase-1 RCT-156 | 1.1653254 | 1.6257813 | 1.0078518 | 1.1461648 | 1.3577694 | 1.2326924 | 1.0604557 |
| Ribosomal protein L13A | 1.2496665 | 1.0832107 | 0.6488808 | 1.353586 | 1.3294154 | 1.1681536 | 1.322898 |
| Phase-1 RCT-258 | 1.0334064 | 1.0249808 | 0.9710539 | 0.6279708 | 0.767957 | 0.9154108 | 1.0304639 |
| Insulin-like growth factor I | 1.193389 | 1.2277659 | 0.746145 | 1.0465671 | 1.523598 | 1.1217859 | 0.7659172 |
| Cytochrome P450 3A1 | 1.078686 | 0.3556989 | 1.6162839 | 1.347688 | 2.7313547 | 1.2340016 | 1.3461218 |
| {Ribosomal protein 16} | 1.0968825 | 0.9357151 | 0.8371645 | 1.2152544 | 1.3048285 | 1.0806589 | 1.1568438 |
| Organic cation transporter 3 | 1.0059855 | 0.9296507 | 0.8878256 | 0.7662027 | 0.9640565 | 0.9413177 | 1.2675036 |
| Calpain 2 | 0.9489632 | 0.937706 | 0.9320765 | 0.9868566 | 0.7580769 | 1.0118699 | 0.9642552 |
| Phase-1 RCT-102 | 0.5487708 | 0.6868162 | 0.5792038 | 0.7270358 | 1.0664263 | 1.0339699 | 0.7432152 |
| Equilbrative nitrobenzylthioinosine-sensitive | 0.974465 | 0.7821439 | 1.2519671 | 0.9247308 | 1.2048652 | 1.0919219 | 0.7761172 |
| nucleoside transporter | | | | | | | |
| Phase-1 RCT-227 | 0.8544015 | 0.7557513 | 0.8849203 | 1.0482994 | 1.0697882 | 1.0422155 | 0.6970717 |
| N-hydroxy-2-acetylaminofluorene sulfotransferase (ST1C1) | 0.9813643 | 0.6150082 | 1.2647359 | 0.906577 | 1.5293059 | 1.25796 | 0.7494679 |
| Cathepsin S | 1.0460138 | 1.0217098 | 1.1510904 | 1.1107199 | 0.7670533 | 0.9894539 | 1.0458715 |
| Apolipoprotein CIII | 0.9783264 | 0.6933701 | 0.9371511 | 0.7255827 | 1.2029722 | 1.2505206 | 1.0762365 |
| Cytochrome P450 2A3 | 1.0063761 | 0.6867659 | 1.8903831 | 1.8538587 | 0.8758176 | 0.8626064 | 0.8671259 |
| Cytochrome P450 2C23 | 1.0574307 | 1.1282346 | 1.1465652 | 1.2303436 | 1.0905607 | 0.8877144 | 0.923413 |
| Apolipoprotein C1 | 0.9407036 | 0.9262682 | 1.0739247 | 1.0105621 | 0.9861431 | 0.8573749 | 0.8767195 |
| Betaine homocysteine methyltransferase (BHMT) | 0.4789906 | 0.4600263 | 0.7332347 | 2.0977316 | 2.4765644 | 1.7445312 | 1.4458791 |
| Paraoxonase 1 | 1.032124 | 1.0188824 | 1.4662071 | 1.0827893 | 1.1573356 | 0.8559121 | 0.733053 |
| Phase-1 RCT-207 | 1.0504447 | 0.9634326 | 0.8151385 | 0.5904763 | 0.7670796 | 0.9001995 | 1.0414084 |
| Cathepsin B | 0.8937037 | 0.8217963 | 0.8442974 | 1.2210201 | 1.2681804 | 1.0698583 | 1.0121799 |
| Phase-1 RCT-144 | 0.969209 | 0.9797341 | 0.8356255 | 0.8137931 | 0.9614666 | 0.912751 | 1.0006049 |
| Alpha-1 microglobulin/bikunin precursor (Ambp) | 1.0172961 | 0.5992527 | 0.8085949 | 1.1226434 | 1.3022913 | 1.0783532 | 0.956655 |
| Cyclin D1 | 1.4106609 | 0.8933275 | 1.389862 | 0.8774915 | 0.6370017 | 0.9652214 | 1.3900472 |
| Presenilin-1 | 0.6992565 | 0.8827213 | 0.7827065 | 1.1165687 | 1.0307845 | 1.2780199 | 0.8606646 |
| Protein tyrosine phosphatase, receptor type, D | 1.3513751 | 1.0255619 | 1.117192 | 1.1322542 | 1.2261975 | 0.8452783 | 0.6228596 |
| Multidrug resistant protein-1 | 1.0699143 | 0.9468519 | 1.3979633 | 1.3041042 | 1.1866733 | 1.2965623 | 1 |
| Cathepsin L, sequence 2 | 1.2703198 | 1.5405949 | 1.3184531 | 1.2043025 | 1.0292462 | 0.8748466 | 0.7826378 |
| JNK1 stress activated protein kinase | 1.218076 | 1.0832772 | 1.3566513 | 1.2980233 | 1.2216706 | 0.7917833 | 0.8552688 |
| Phase-1 RCT-43 | 1.0853279 | 1.4133632 | 0.9445146 | 0.7758622 | 1.1117692 | 1.056887 | 1.1519431 |
| Tissue plasminogen activator | 0.8917441 | 0.8347132 | 0.905597 | 0.8676603 | 0.7834409 | 0.8610902 | 0.7294761 |
| Ubiquitin conjugating enzyme (RAD 6 | 1.1445509 | 0.8334487 | 1.0203898 | 0.9655548 | 1.3145353 | 1.1129712 | 1.0816532 |
| homologue) | | | | | | | |
| Phase-1 RCT-173 | 1.0492277 | 0.7612544 | 0.8040261 | 0.6118906 | 0.7962228 | 0.8611472 | 1.1721451 |
| Ornithine decarboxylase | 1.1500596 | 0.9486613 | 1.1029445 | 1.0148407 | 0.7412745 | 1.1134979 | 1.3329836 |
| Zinc finger protein | 0.983519 | 1.0812854 | 1.1095513 | 0.6482025 | 0.8435676 | 0.8352441 | 0.887145 |
| CDK108 | 0.8581975 | 0.7188312 | 0.8686774 | 0.9259129 | 0.8490878 | 0.9708504 | 0.8018494 |
| Phase-1 RCT-117 | 0.9316769 | 0.9098469 | 1.3896629 | 0.8876585 | 1.3754059 | 1.4253294 | 1.2117736 |
| {Phase-1 RCT 98} | 0.8899492 | 1.040548 | 0.9469302 | 1.0791463 | 0.8890688 | 1.0413487 | 0.8809245 |
| Aquaporin-3 (AQP3) | 0.9642802 | 0.9481025 | 0.9807515 | 0.8447436 | 0.6627092 | 0.927202 | 0.8128711 |
| Cholesterol esterase | 0.9277684 | 0.5830213 | 1.1673232 | 1.0481422 | 1.055823 | 0.7367486 | 0.6476693 |
| Ribosomal protein S17 | 1.0338591 | 1.0603846 | 0.9413181 | 0.9958784 | 1.1306316 | 0.8374724 | 0.8872283 |
| Poly(ADP-ribose) polymerase | 0.9398639 | 0.8855109 | 1.069943 | 0.8368996 | 0.8770677 | 1.100264 | 1.1113693 |
| Annexin V | 1.2057784 | 1.0499079 | 1.0097718 | 0.8820123 | 0.8453419 | 1.031034 | 0.9638959 |
| Phase-1 RCT-33 | 0.9036369 | 0.7825428 | 0.5991853 | 1.3823099 | 1.0233452 | 1.3413688 | 1.3698196 |
| Phase-1 RCT-61 | 0.9560433 | 1.0139822 | 0.9000632 | 0.6545223 | 0.7237802 | 0.9718466 | 1.1931238 |
| Retinol dehydrogenase type III | 1.3836842 | 0.6953986 | 0.7914044 | 1.0525593 | 1.5091438 | 1.0837955 | 1.263725 |
| ATPase inhibitor (rat mitochondrial IF1 protein) | 0.882874 | 0.8577039 | 0.9897327 | 0.9544224 | 1.2323565 | 0.9308765 | 0.901565 |
| Thymidylate synthase | 1.0768179 | 1.1205926 | 1.1415595 | 0.8543173 | 0.6681171 | 0.8665888 | 0.9000392 |
| Interleukin-18 | 0.9442239 | 1.1314511 | 1.035866 | 0.7316818 | 0.5774962 | 0.839245 | 1.0816627 |
| Lecithin: cholesterol acyltransferase | 1.1643548 | 0.8942171 | 0.9654762 | 1.1536632 | 1.2160217 | 1.100295 | 1.2820234 |
| Contrapsin-like protease inhibitor (CPi-21) | 1.0849211 | 0.6397445 | 0.835081 | 1.003398 | 0.9856922 | 0.8203927 | 0.535546 |
| Proliferating cell nuclear antigen gene | 0.8773213 | 0.9599047 | 1.0002953 | 0.8131774 | 0.5755484 | 0.8527367 | 1.0411046 |
| Phase-1 RCT-230 | 0.8576123 | 1.1517332 | 0.8886613 | 0.7029139 | 0.6293154 | 0.801657 | 0.9087213 |
| Cytochrome P450 2D18 | 1.1499196 | 0.9433174 | 1.0243144 | 1.1745298 | 1.0357163 | 1.1005872 | 1.0345931 |
| Phase-1 RCT-48 | 0.9492693 | 0.8780756 | 0.9292712 | 1.0744971 | 1.215841 | 1.4190494 | 1.0154947 |
| Phase-1 RCT-292 | 0.8838115 | 1.1092736 | 1.0092523 | 0.8476971 | 0.8708079 | 0.9545222 | 0.9684958 |
| Arginosuccinate synthetase 1 | 1.2223109 | 0.8240839 | 1.258158 | 1.8455293 | 1.7107819 | 1.2971047 | 1.0130568 |
| C-reactive protein | 1.6339247 | 0.9810287 | 1.4572169 | 1.0421163 | 1.2921678 | 0.8876142 | 0.6068611 |

| | CYCA | DEX | DEX | DEX | CYCA | DEX |
|---|---|---|---|---|---|---|
| Compound-Dose | 80 | 30 | 8 | 8 | 80 | 30 |
| Animal Number | 438 | 1359 | 1347 | 1348 | 439 | 1357 |
| Liver Toxicity Necrosis Classification | no | no | no | no | no | no |
| Gene Name | | | | | | |
| Osteoactivin | 1.1373097 | 1.168433 | 0.8926441 | 1.0287001 | 1 | 0.8681322 |
| Stathmin | 1.2221406 | 1.0303411 | 1.057627 | 1.0630435 | 1.0995694 | 1.0461903 |
| Calpactin I heavy chain | 1.2509696 | 1.2713994 | 1.1356795 | 1.1089665 | 1.1621914 | 1.0293918 |
| Phase-1 RCT-179 | 1.1354861 | 0.9598793 | 0.8659737 | 0.8496479 | 0.8287516 | 0.9164758 |
| Phase-1 RCT-107 | 0.6709359 | 1.0990493 | 1.8462869 | 1.017612 | 0.7180358 | 1.6642781 |
| Heme oxygenase | 1.9947127 | 0.8094142 | 0.8310824 | 1.2104739 | 1.1638529 | 0.7467025 |
| Collagen type II | 1.292575 | 1.1667099 | 0.9738999 | 1.0804911 | 1.4139172 | 1.3099663 |
| Phase-1 RCT-81 | 0.8884199 | 0.9340932 | 0.9932361 | 0.9449348 | 0.8668365 | 0.8784791 |
| Glycine methyltransferase | 0.548668 | 1.1235883 | 1.9821277 | 1.0131313 | 0.6512998 | 1.7141715 |
| Cystatin C | 0.8876274 | 0.8226437 | 0.8849866 | 0.8363519 | 0.7316357 | 1.0777305 |
| Melanoma-associated antigen ME491 | 0.9168849 | 1.042141 | 1.0566673 | 0.9592373 | 0.9997298 | 0.9929628 |
| Phase-1 RCT-146 | 1.0965767 | 1.008534 | 0.9149602 | 1.1100153 | 1.1321158 | 0.9664224 |
| Gamma-actin, cytoplasmic | 1.0087211 | 0.9177746 | 0.8829677 | 0.9507126 | 0.9547366 | 1.0866205 |
| Adenine nucleotide translocator 1 | 0.7602497 | 0.9053068 | 1.0871047 | 0.9653234 | 0.8601927 | 0.9797443 |
| Thymosin beta-10 | 1.1644214 | 0.852462 | 1.7925421 | 0.9718541 | 1.0608912 | 1.3050677 |
| Insulin-like growth factor I, exon 6 | 1.1524009 | 0.8497729 | 0.6310394 | 1.0581223 | 1.016287 | 0.6826754 |
| Phase-1 RCT-78 | 0.9427422 | 1.1528896 | 1.1179467 | 0.9762253 | 0.8515819 | 0.8838849 |
| Alpha-2-microglobulin | 0.3772149 | 0.9657357 | 1.1425353 | 0.9413459 | 0.3815329 | 1.3331392 |
| High affinity IgE receptor gamma chain | 1.2187135 | 0.9807501 | 0.8055319 | 0.8812372 | 0.9411922 | 0.8054327 |
| (FcERIgamma) | | | | | | |
| 60S ribosomal protein L6 | 1.1998192 | 0.890112 | 1.0984703 | 0.9352627 | 1.0070952 | 1.0975139 |
| IgE binding protein | 1.3736777 | 1.0521382 | 1.0393801 | 0.9919637 | 0.9135652 | 1.0961411 |
| Phase-1 RCT-149 | 1.1265751 | 1.1594212 | 1.0356163 | 1.1369904 | 1.108202 | 0.7734514 |
| Cytochrome P450 2C11 | 1.1869307 | 1.1749051 | 1.2747794 | 1.1837368 | 1.2080069 | 1.1368362 |
| Uncoupling protein 2 | 1.451889 | 0.8754012 | 1.0968854 | 0.9301615 | 1.2299707 | 1.0899016 |
| Cofilin | 0.8164281 | 1.0590024 | 1.0057212 | 1.0235351 | 0.8268966 | 0.9999152 |
| Alpha-tubulin | 0.9929797 | 0.82721 | 1.2509701 | 0.9313134 | 0.9883693 | 1.092825 |
| Stearyl-CoA desaturase, liver | 0.5262004 | 1.581012 | 0.8285912 | 1.3273739 | 0.9754015 | 0.5498952 |
| Glutathione S-transferase theta-1 | 0.8808028 | 0.9901292 | 0.9290785 | 0.9990611 | 1.148066 | 0.8885713 |
| alpha-1,2-fucosyltrans-ferase | 1.5931386 | 0.8941929 | 0.8706939 | 0.9929878 | 1.4794458 | 0.7595549 |
| Phase-1 RCT-138 | 1.184448 | 0.919156 | 0.9399467 | 0.9819252 | 1.0218083 | 0.8606784 |
| Phase-1 RCT-295 | 1.0904615 | 1.0470667 | 1.1357812 | 0.9502921 | 0.9222117 | 1.0473233 |
| Phase-1 RCT-164 | 0.9583131 | 0.8597189 | 0.7256435 | 0.9494314 | 0.9506964 | 0.7202885 |
| Alpha-2-macroglobulin, sequence 2 | 0.9382026 | 0.8487625 | 1.0689446 | 0.9788931 | 0.9017154 | 0.9594566 |
| Biliverdin reductase | 1.1471075 | 2.2456784 | 1.0223019 | 1.111059 | 1.1112258 | 1.3207167 |
| Phase-1 RCT-121 | 1.0572119 | 0.864018 | 0.9207063 | 0.9029191 | 0.8022444 | 1.0610038 |
| Dynamin-1 (D100) | 0.7628707 | 1.0173571 | 1.0940012 | 0.908018 | 0.84281 | 0.9644233 |
| Phase-1 RCT-192 | 1.0427132 | 1.0145966 | 0.9854716 | 0.9729747 | 0.9462553 | 0.8946574 |
| Fatty acid synthase | 0.4124102 | 0.8593862 | 2.104933 | 0.8511291 | 0.6867376 | 1.2813133 |
| Phase-1 RCT-211 | 1.4195893 | 1.0325742 | 1.1168119 | 1.05318 | 1.187761 | 0.9609213 |
| Ribosomal protein S9 | 1.0866119 | 0.9403741 | 1.016157 | 0.86572 | 1.0812929 | 0.897826 |
| NADP-dependent isocitrate dehydrogenase, | 1.0077947 | 0.9222419 | 0.7331664 | 0.8853303 | 1.0515355 | 0.7427957 |
| cytosolic | | | | | | |
| Phase-1 RCT-57 | 0.9392674 | 1.3678645 | 1.063811 | 0.9695571 | 0.9437738 | 1.0281683 |
| Phase-1 RCT-36 | 1.2800962 | 1.040352 | 1.0317155 | 0.9966174 | 1.1437129 | 1.2106204 |
| Apolipoprotein All | 1.1408312 | 0.7857571 | 0.4198115 | 0.8030411 | 1.6144732 | 0.4911386 |
| Phase-1 RCT-68 | 1.0134813 | 1.1839001 | 1.227586 | 1.0449538 | 0.9710295 | 1.1847967 |
| Multidrug resistant protein-2 | 1.3565736 | 1.1356161 | 1.1907235 | 1.2142371 | 1.1471981 | 1.3725097 |
| Phase-1 RCT-39 | 1.4895387 | 0.8476628 | 0.9167696 | 1.0717045 | 1.0650585 | 0.9074565 |
| NIPK | 1.1440485 | 0.9258609 | 0.8896686 | 1.1425567 | 1.0498401 | 0.7727542 |
| F1-ATPase beta subunit | 0.7662715 | 0.9797249 | 1.5758704 | 1.0321867 | 0.8806374 | 1.2360371 |
| Elongation factor-1 alpha | 1.0092345 | 0.8911947 | 1.1796876 | 1.0185589 | 0.7764618 | 1.1446123 |
| Phase-1 RCT-24 | 1.0934291 | 0.9590055 | 1.4172795 | 0.9930911 | 1.0175707 | 1.3064494 |
| Beta-tubulin, class I | 0.8472833 | 0.8776367 | 1.5585313 | 0.9808783 | 0.7209882 | 1.1507015 |
| 14-3-3 zeta | 1.0916127 | 0.9940366 | 1.0484194 | 1.0651486 | 1.09253 | 1.0034224 |
| Alpha-prothymosin | 0.8248938 | 1.1969184 | 1.2513101 | 1.125395 | 0.7791795 | 1.2206609 |
| Phase-1 RCT-58 | 1.3390439 | 0.7817764 | 0.4487111 | 0.91712 | 0.9510657 | 0.7584435 |
| Phase-1 RCT-290 | 0.5071215 | 1.8421942 | 2.542163 | 1.230475 | 0.673233 | 1.2550104 |
| Transitional endoplasmic reticulum ATPase | 0.8053101 | 0.9347181 | 0.9991449 | 0.9905506 | 0.865678 | 1.0197226 |
| Beta-actin | 1.1260333 | 0.8731179 | 0.8649273 | 0.980727 | 0.8719199 | 1.3548365 |
| Thioredoxin-1 (Trx1) | 1.235957 | 0.8300796 | 0.8513024 | 0.8479866 | 1.0100908 | 0.6659823 |
| Phase-1 RCT-174 | 0.969199 | 1.1887586 | 0.9752832 | 0.9931502 | 0.8998229 | 0.8186175 |
| Beta-actin, sequence 2 | 1.3644415 | 1.0242997 | 0.9435248 | 0.9691865 | 1.1149783 | 1.0069482 |
| Phase-1 RCT-109 | 1.4095066 | 0.992094 | 1.0570976 | 0.9463994 | 1.1299595 | 1.0810713 |
| Phase-1 RCT-154 | 1.0178943 | 1.0624061 | 1.0380309 | 1.0440187 | 1.0355182 | 1.0736246 |
| Hypoxanthine-guanine | 0.822448 | 1.0078133 | 1.3325127 | 0.9160264 | 1.0284543 | 1.2086903 |
| phosphoribosyltransferase | | | | | | |
| Phase-1 RCT-213 | 0.9045925 | 1.1077468 | 1.1061025 | 1.0422751 | 0.9869179 | 1.0743072 |
| Phase-1 RCT-260 | 1.273741 | 1.0030396 | 0.9100332 | 1.1085892 | 0.9434566 | 0.8180227 |
| Phase-1 RCT-12 | 1.0480716 | 0.8760669 | 1.2359688 | 0.9689562 | 0.9357342 | 1.1762588 |
| Phase-1 RCT-293 | 1.2823626 | 0.9061689 | 0.7482792 | 0.961253 | 1.2086923 | 0.7504225 |
| Ribosomal protein S8 | 1.0990369 | 0.9089149 | 1.1514633 | 0.8711389 | 0.8179376 | 0.7852554 |
| Pyruvate kinase, muscle | 1.3422297 | 0.9467503 | 1.2388217 | 1.1138034 | 1.1754122 | 1.329256 |
| Nucleoside diphosphate kinase beta isoform | 1.0795428 | 1.1544698 | 1.1175275 | 1.00193 | 0.9675254 | 1.0142729 |
| p55CDC | 0.9174748 | 1.0879633 | 1.0196903 | 1.0606078 | 1.2282223 | 0.7553567 |
| Phase-1 RCT-156 | 1.0469296 | 0.9753648 | 0.9279439 | 0.9113069 | 0.8932748 | 1.0348014 |
| Ribosomal protein L13A | 1.5191462 | 1.0357401 | 1.083524 | 1.0512401 | 1.1970937 | 1.1658009 |
| Phase-1 RCT-258 | 0.899464 | 1.0142124 | 0.961634 | 0.942185 | 0.7228025 | 1.0048363 |
| Insulin-like growth factor I | 0.9400944 | 0.8881954 | 0.7300319 | 0.8803169 | 0.7554823 | 0.7114102 |
| Cytochrome P450 3A1 | 0.8477613 | 0.8937422 | 0.9561638 | 0.6950997 | 0.6006416 | 0.8112136 |
| {Ribosomal protein 16} | 1.2227399 | 1.0467294 | 1.5725636 | 0.9921544 | 0.9393421 | 1.3305948 |
| Organic cation transporter 3 | 1.2486745 | 0.884895 | 1.1052897 | 0.8601493 | 1.0266716 | 0.9700314 |
| Calpain 2 | 1.0484521 | 1.0818881 | 1.2752059 | 1.0300834 | 1.0274218 | 1.1811955 |
| Phase-1 RCT-102 | 0.6260704 | 0.8180264 | 0.5548869 | 0.9301394 | 0.5836417 | 0.6843917 |
| Equilbrative nitrobenzylthioinosine-sensitive | 0.9107553 | 0.8533841 | 0.5458135 | 0.8688201 | 0.7949209 | 0.5156249 |
| nucleoside transporter | | | | | | |
| Phase-1 RCT-227 | 0.8148055 | 1.2152271 | 1.1552309 | 0.9487746 | 0.7082246 | 0.9308952 |
| N-hydroxy-2-acetylaminofluorene sulfotransferase (ST1C1) | 0.7988989 | 0.8299382 | 0.4888163 | 0.82139 | 0.7470667 | 0.4959706 |
| Cathepsin S | 1.5310528 | 0.7880346 | 0.7945166 | 0.7615979 | 1.141958 | 0.7020939 |
| Apolipoprotein CIII | 0.7853958 | 1.0372055 | 1.0454813 | 0.896011 | 0.8803638 | 1.0357946 |
| Cytochrome P450 2A3 | 0.4314712 | 0.7285422 | 0.909916 | 1.1106101 | 0.9831398 | 0.7122763 |
| Cytochrome P450 2C23 | 0.7918571 | 0.8382943 | 0.3326177 | 0.8877043 | 0.8980203 | 0.6046573 |
| Apolipoprotein C1 | 0.6618601 | 1.0103782 | 0.6651555 | 0.8898788 | 0.7098429 | 0.6883939 |
| Betaine homocysteine methyltransferase (BHMT) | 0.3672821 | 1.8711014 | 3.0408347 | 1.2547274 | 0.6143343 | 1.2194722 |
| Paraoxonase 1 | 0.768317 | 0.7616931 | 0.6068233 | 0.8613967 | 0.8205603 | 0.6017869 |
| Phase-1 RCT-207 | 0.9298006 | 1.1291574 | 1.1841198 | 1.0577494 | 0.9904526 | 1.1036496 |
| Cathepsin B | 1.1136122 | 1.0458587 | 0.937949 | 1.0225977 | 1.1312598 | 1.1296322 |
| Phase-1 RCT-144 | 0.9493954 | 0.9834796 | 0.9979191 | 0.9537398 | 1.0227901 | 1.0285193 |
| Alpha-1 microglobulin/bikunin precursor (Ambp) | 0.873772 | 0.9371322 | 0.9545603 | 0.8901222 | 0.795792 | 0.8863753 |
| Cyclin D1 | 0.907176 | 0.7401671 | 1.0217454 | 0.8874148 | 0.9812383 | 0.857685 |
| Presenilin-1 | 1.1319015 | 0.9994041 | 0.4931508 | 0.7001473 | 0.9678403 | 0.4451235 |
| Protein tyrosine phosphatase, receptor type, D | 0.7038342 | 1.0203663 | 0.5873286 | 1.0026922 | 0.6813473 | 0.6626239 |
| Multidrug resistant protein-1 | 1.2680602 | 1.3118036 | 1.2572452 | 1.4359819 | 1.1089159 | 1.6939944 |
| Cathepsin L, sequence 2 | 1.3612336 | 0.9605257 | 1.1354592 | 1.0420544 | 1.0214374 | 1.2092525 |
| JNK1 stress activated protein kinase | 0.5181539 | 0.8379765 | 2.2034755 | 1.1260844 | 0.8660355 | 1.9482772 |
| Phase-1 RCT-43 | 0.9997548 | 0.940602 | 0.9602948 | 0.8617249 | 0.9066359 | 0.9051095 |
| Tissue plasminogen activator | 1.0097779 | 1.3477398 | 1.0354437 | 1.0102112 | 0.7997062 | 1.5838435 |
| Ubiquitin conjugating enzyme (RAD 6 | 1.0739564 | 0.8605588 | 1.0687081 | 0.899923 | 0.9341322 | 0.9279984 |
| homologue) | | | | | | |
| Phase-1 RCT-173 | 0.8481727 | 1.1465967 | 1.3401563 | 1.0833035 | 0.9636456 | 1.0311909 |
| Ornithine decarboxylase | 0.8564838 | 1.381269 | 1.108185 | 1.2241372 | 0.9493741 | 1.2058322 |
| Zinc finger protein | 0.9247244 | 1.0007237 | 0.9269436 | 0.9999408 | 0.8498479 | 0.9122886 |
| CDK108 | 0.8645644 | 1.209934 | 1.0813909 | 1.1745211 | 0.9150673 | 1.0304205 |
| Phase-1 RCT-117 | 1.1183646 | 0.5893585 | 0.6128073 | 0.8066956 | 0.9617502 | 0.8728184 |
| {Phase-1 RCT 98} | 0.8093642 | 1.2049369 | 1.2678548 | 1.0495706 | 0.8243842 | 1.7796776 |
| Aquaporin-3 (AQP3) | 0.8562856 | 1.12413 | 1.1251388 | 1.0184329 | 0.9512967 | 0.9442074 |
| Cholesterol esterase | 0.4667917 | 0.6425217 | 0.6432579 | 0.8417985 | 0.6185527 | 0.5353763 |
| Ribosomal protein S17 | 1.0195711 | 1.0240529 | 1.1324502 | 0.9665769 | 0.7829655 | 0.8921167 |
| Poly(ADP-ribose) polymerase | 0.9447051 | 1.0382776 | 1.2817653 | 0.9562513 | 1.0369065 | 1.1786635 |
| Annexin V | 0.8928967 | 0.9631725 | 0.8389845 | 0.9176046 | 1.1249336 | 0.9352757 |
| Phase-1 RCT-33 | 1.3924003 | 1.0196495 | 1.0478865 | 0.9930534 | 1.1889304 | 1.1979105 |
| Phase-1 RCT-61 | 1.1097097 | 0.9120211 | 0.5451261 | 0.9437482 | 1.0297428 | 0.8543657 |
| Retinol dehydrogenase type III | 1.1076088 | 0.8553115 | 0.9651327 | 0.9975113 | 0.8473548 | 0.7478024 |
| ATPase inhibitor (rat mitochondrial IF1 protein) | 0.7156372 | 0.8013049 | 0.5192791 | 0.8298688 | 0.5881522 | 0.6549297 |
| Thymidylate synthase | 0.8606147 | 1.2263026 | 0.8806017 | 0.82043 | 1.0895989 | 0.7643533 |
| Interleukin-18 | 1.0063198 | 0.9260005 | 1.121985 | 0.973629 | 1.0677767 | 0.9786008 |
| Lecithin: cholesterol acyltransferase | 1.2180676 | 1.1115756 | 1.2543334 | 1.0068926 | 1.0189469 | 1.2700537 |
| Contrapsin-like protease inhibitor (CPi-21) | 0.9583862 | 1.4135526 | 1.0203757 | 1.0682054 | 0.6108701 | 1.8777237 |
| Proliferating cell nuclear antigen gene | 1.0241978 | 0.9754206 | 1.0367689 | 0.9920989 | 1.1028612 | 0.98596 |
| Phase-1 RCT-230 | 1.2858104 | 1.0030453 | 1.0009565 | 1.1922292 | 1.0886387 | 0.9334081 |
| Cytochrome P450 2D18 | 0.9770908 | 1.3896873 | 1.147087 | 1.0238819 | 0.9431716 | 1.2109227 |
| Phase-1 RCT-48 | 0.7833505 | 1.0926317 | 0.8444762 | 1.079283 | 0.8117194 | 0.6433018 |
| Phase-1 RCT-292 | 0.9395645 | 1.096095 | 1.0346955 | 1.0258346 | 1.0116643 | 0.8749687 |
| Arginosuccinate synthetase 1 | 0.4934185 | 1.0595526 | 1.941308 | 1.0978912 | 0.8576392 | 1.5917776 |
| C-reactive protein | 0.7732859 | 0.9729796 | 0.5971896 | 0.9643968 | 0.6559664 | 0.7188557 |

| | DEX | DEX | DIF | DIF | DIF | DIF | DIF |
|---|---|---|---|---|---|---|---|
| Compound-Dose | 30 | 8 | 25 | 25 | 25 | 100 | 100 |
| Animal Number | 1358 | 1349 | 247 | 248 | 249 | 257 | 258 |
| Liver Toxicity Necrosis Classification | no | no | no | no | no | no | no |
| Gene Name | | | | | | | |
| Osteoactivin | 1.1418818 | 1.0302615 | 0.9134798 | 1.0858576 | 0.8827833 | 0.9802117 | 1.0788586 |
| Stathmin | 1.0661529 | 0.9960029 | 1.0041112 | 1.0256697 | 0.9348507 | 0.9613327 | 0.9391595 |
| Calpactin I heavy chain | 1.2262361 | 1.0574887 | 1.0828649 | 1.0767751 | 1.3592842 | 1.2440219 | 1.343733 |
| Phase-1 RCT-179 | 0.9421471 | 0.9005984 | 0.8029198 | 0.9245838 | 0.8659027 | 0.8717321 | 0.9043346 |
| Phase-1 RCT-107 | 0.9860987 | 0.9748602 | 0.973236 | 1.1315227 | 1.2942916 | 1.4735761 | 1.0875403 |
| Heme oxygenase | 1.0959599 | 1.3497748 | 0.9250774 | 1.0467008 | 1.036567 | 1.0112636 | 1.2160974 |
| Collagen type II | 0.9814044 | 1.0324546 | 1.6223946 | 0.9999999 | 1.1653004 | 1.5549595 | 1.3487002 |
| Phase-1 RCT-81 | 1.0460094 | 1.0537293 | 1.026234 | 1.1672418 | 1.0734495 | 1.2549835 | 1.2211881 |
| Glycine methyltransferase | 1.0052238 | 0.9429279 | 1.069029 | 1.1482091 | 1.3459789 | 1.4983052 | 1.174554 |
| Cystatin C | 0.9245112 | 0.8758853 | 0.9605542 | 1.0492852 | 0.9627404 | 0.9974733 | 0.9412975 |
| Melanoma-associated antigen ME491 | 1.1281337 | 1.1246439 | 0.8820722 | 1.117912 | 1.0863479 | 1.0479196 | 1.1855794 |
| Phase-1 RCT-146 | 1.1122983 | 1.0494133 | 1.2343392 | 1.2217687 | 1.0031977 | 0.9142811 | 1.0602461 |
| Gamma-actin, cytoplasmic | 0.9446226 | 0.9548147 | 1.4194037 | 1.0494705 | 1.0862042 | 1.0007329 | 0.8897873 |
| Adenine nucleotide translocator 1 | 1.0569977 | 0.9934108 | 0.8094848 | 1.0537848 | 1.141217 | 0.9584677 | 1.117301 |
| Thymosin beta-10 | 0.8097717 | 0.821438 | 0.7918813 | 0.7825609 | 0.8647845 | 0.8018026 | 0.7774236 |
| Insulin-like growth factor I, exon 6 | 0.7500891 | 0.9396342 | 0.7363164 | 0.7960733 | 0.5595143 | 0.6983735 | 0.6395076 |
| Phase-1 RCT-78 | 1.2335231 | 1.0785503 | 1.0323254 | 0.916931 | 1.030719 | 1.0162021 | 1.1842184 |
| Alpha-2-microglobulin | 0.8450784 | 0.7266874 | 0.6084669 | 0.61351 | 0.7378305 | 0.7121453 | 0.6189379 |
| High affinity IgE receptor gamma chain | 1.0533477 | 0.999752 | 0.7705677 | 1.2024305 | 1.0949018 | 1.1929986 | 1.0814949 |
| (FcERIgamma) | | | | | | | |
| 60S ribosomal protein L6 | 0.8908617 | 1.0054346 | 0.7870476 | 0.9838848 | 0.8367958 | 0.9330581 | 1.0072525 |
| IgE binding protein | 0.9631939 | 0.9747619 | 0.8528187 | 1.0853467 | 0.9732043 | 0.9751086 | 1.0570742 |
| Phase-1 RCT-149 | 1.1466366 | 1.1034437 | 1.559252 | 1.2775365 | 1.1369218 | 1.1961715 | 1.2918023 |
| Cytochrome P450 2C11 | 1.3866265 | 1.2648478 | 1.0240912 | 0.8336794 | 1.2396117 | 0.9675527 | 1.0590408 |
| Uncoupling protein 2 | 0.763873 | 0.8995098 | 0.9593027 | 0.7792972 | 0.9003245 | 0.7752812 | 0.7434526 |
| Cofilin | 1.1241914 | 0.8360431 | 1.1914866 | 1.3156139 | 1.0796108 | 1.2713027 | 1.4095652 |
| Alpha-tubulin | 0.8587909 | 1.0029331 | 1.143198 | 0.965915 | 1.2449235 | 0.9727722 | 1.0027493 |
| Stearyl-CoA desaturase, liver | 1.8186162 | 1.2651758 | 1.2354568 | 1.0438006 | 1.7766961 | 1.026311 | 1.9728469 |
| Glutathione S-transferase theta-1 | 0.9801158 | 1.0558234 | 0.7954495 | 0.9714393 | 1.0449287 | 0.8869818 | 0.8814598 |
| alpha-1,2-fucosyltrans-ferase | 0.7038544 | 0.9427272 | 0.5083948 | 0.4796748 | 0.5238005 | 0.4771789 | 0.4936164 |
| Phase-1 RCT-138 | 0.9797315 | 0.9553008 | 0.9235792 | 1.1097068 | 0.9776304 | 1.1067604 | 0.9306929 |
| Phase-1 RCT-295 | 1.1205373 | 1.0548445 | 0.8472527 | 1.0042217 | 1.0680013 | 0.9619931 | 1.0068103 |
| Phase-1 RCT-164 | 0.7897846 | 0.8061454 | 1.0012428 | 0.941135 | 0.8712708 | 0.9787451 | 0.9365926 |
| Alpha-2-macroglobulin, sequence 2 | 0.9011146 | 0.9160372 | 0.9260933 | 0.910606 | 0.8463839 | 0.8669934 | 0.8773653 |
| Biliverdin reductase | 1.1707364 | 1.1155549 | 1.343723 | 1.286358 | 1.445571 | 1.2529286 | 1.5179645 |
| Phase-1 RCT-121 | 0.8964883 | 0.892643 | 1.1409723 | 0.862186 | 0.7902649 | 0.9170046 | 0.856728 |
| Dynamin-1 (D100) | 1.0707159 | 1.0014137 | 0.843387 | 0.9072521 | 0.8653567 | 1.0184603 | 0.9598382 |
| Phase-1 RCT-192 | 0.9956306 | 0.9679415 | 0.9512172 | 1.0530702 | 0.9720149 | 0.9708058 | 1.0793461 |
| Fatty acid synthase | 1.1650151 | 0.9941424 | 2.0251634 | 0.9908375 | 2.2287927 | 0.9015297 | 1.0707119 |
| Phase-1 RCT-211 | 1.0525991 | 1.0598507 | 0.7391959 | 0.8506943 | 0.916015 | 0.8257656 | 0.8609575 |
| Ribosomal protein S9 | 1.0156658 | 0.9444869 | 0.982283 | 1.0143889 | 1.0319083 | 0.8403275 | 0.9612945 |
| NADP-dependent isocitrate dehydrogenase, | 0.9358992 | 0.9319758 | 0.9684701 | 1.1348742 | 1.0175914 | 0.9694332 | 1.0473881 |
| cytosolic | | | | | | | |
| Phase-1 RCT-57 | 1.1405127 | 1.0025879 | 1.2658811 | 1.0305854 | 1.1222717 | 1.1415013 | 1.1334682 |
| Phase-1 RCT-36 | 1.0878614 | 1.0563667 | 1.0544852 | 0.899322 | 0.9199361 | 0.811471 | 0.9034221 |
| Apolipoprotein All | 0.727478 | 0.788006 | 0.72355 | 0.5419063 | 0.6556432 | 0.7411756 | 0.6242764 |
| Phase-1 RCT-68 | 1.3278276 | 1.0870974 | 1.0274911 | 1.0299733 | 1.1829813 | 1.0733877 | 1.1247232 |
| Multidrug resistant protein-2 | 1.0546961 | 1.1345975 | 1.1036977 | 1.092324 | 1.1124306 | 1.0686144 | 1.0266072 |
| Phase-1 RCT-39 | 0.9103508 | 0.9732536 | 0.8735966 | 1.1105481 | 0.9938413 | 0.8827903 | 0.9560426 |
| NIPK | 0.8366451 | 1.0332506 | 1.0112019 | 0.8298403 | 0.5991254 | 0.6888776 | 0.663653 |
| F1-ATPase beta subunit | 0.9181134 | 0.9768769 | 0.8209402 | 1.0598853 | 0.9445921 | 0.9610047 | 1.090107 |
| Elongation factor-1 alpha | 0.8564375 | 1.1107513 | 0.8968803 | 0.9453154 | 0.9335704 | 1.1731606 | 1.0707573 |
| Phase-1 RCT-24 | 0.9715312 | 1.0692143 | 1.3566163 | 1.1453048 | 1.435908 | 1.1498568 | 1.1355737 |
| Beta-tubulin, class I | 0.962209 | 0.9051355 | 1.8600973 | 1.1472635 | 1.3523692 | 1.124271 | 1.2748728 |
| 14-3-3 zeta | 1.0543782 | 1.1293201 | 1.1384587 | 1.0642174 | 1.0845398 | 1.0970657 | 1.0479724 |
| Alpha-prothymosin | 1.182549 | 0.9512425 | 1.1246077 | 1.4742233 | 1.2131952 | 1.4826673 | 1.4080604 |
| Phase-1 RCT-58 | 0.9371041 | 0.8434526 | 0.9044239 | 0.8092644 | 0.6154229 | 0.8314195 | 0.524332 |
| Phase-1 RCT-290 | 1.4882594 | 0.9801048 | 1.3108894 | 1.2374017 | 1.017092 | 1.0066966 | 1.1795152 |
| Transitional endoplasmic reticulum ATPase | 0.9874164 | 1.0121182 | 1.2303447 | 0.9720562 | 1.1689459 | 0.9798908 | 1.026688 |
| Beta-actin | 0.8511382 | 1.063662 | 1.1371609 | 0.942909 | 1.0125045 | 0.9148535 | 0.8092046 |
| Thioredoxin-1 (Trx1) | 0.8208509 | 0.861599 | 0.5854623 | 0.8084102 | 0.7185597 | 0.7094124 | 0.6707681 |
| Phase-1 RCT-174 | 1.1747421 | 1.0330352 | 0.8128052 | 1.1139053 | 1.2886201 | 1.004065 | 1.3626654 |
| Beta-actin, sequence 2 | 1.0427792 | 0.9228895 | 0.8796874 | 1.2241431 | 1.0241793 | 1.0360639 | 1.153502 |
| Phase-1 RCT-109 | 1.0056187 | 0.8937542 | 0.7158874 | 0.8486533 | 0.9063498 | 0.8832561 | 0.9008338 |
| Phase-1 RCT-154 | 1.0267506 | 0.9824767 | 1.1020063 | 1.3082018 | 1.0872426 | 1.0598809 | 1.0488687 |
| Hypoxanthine-guanine | 0.9485482 | 0.9572228 | 0.9901889 | 1.0062994 | 1.1204743 | 1.0056595 | 1.2431145 |
| phosphoribosyltransferase | | | | | | | |
| Phase-1 RCT-213 | 1.0722929 | 1.0292861 | 1.2234968 | 0.9684873 | 0.857576 | 0.8993807 | 0.9304098 |
| Phase-1 RCT-260 | 0.9753857 | 0.9392061 | 0.9863078 | 0.9641823 | 0.9150471 | 0.8443008 | 0.8507803 |
| Phase-1 RCT-12 | 0.9583873 | 0.9672071 | 1.2116052 | 1.2062598 | 1.3618164 | 1.1417536 | 1.2518828 |
| Phase-1 RCT-293 | 1.027835 | 0.9734273 | 0.9639453 | 0.9740681 | 1.1000922 | 1.1424673 | 0.9766903 |
| Ribosomal protein S8 | 0.9040608 | 0.9206201 | 0.7716033 | 1.0899737 | 0.9841762 | 0.9056741 | 0.9947605 |
| Pyruvate kinase, muscle | 0.9634752 | 1.0995954 | 1.1550775 | 1.1114538 | 0.9587293 | 1.076931 | 0.9145396 |
| Nucleoside diphosphate kinase beta isoform | 1.0003064 | 1.0749851 | 1.0030797 | 1.0543342 | 1.2017149 | 1.0431689 | 1.2036337 |
| p55CDC | 1.0527911 | 1.1495955 | 1.0658807 | 0.9900763 | 0.8692483 | 1.3405198 | 0.9577152 |
| Phase-1 RCT-156 | 1.0763147 | 1.0056305 | 0.9760208 | 1.1129731 | 0.9950463 | 1.0077113 | 1.0591596 |
| Ribosomal protein L13A | 0.8784306 | 1.0379046 | 0.8292339 | 0.7193449 | 0.8400526 | 0.8612678 | 0.7850265 |
| Phase-1 RCT-258 | 1.005884 | 0.933702 | 1.1367239 | 1.0927899 | 0.9667029 | 1.0000143 | 1.1169755 |
| Insulin-like growth factor I | 0.7802773 | 0.9569349 | 0.7112828 | 0.812895 | 0.5261757 | 0.7571422 | 0.7027489 |
| Cytochrome P450 3A1 | 0.7505121 | 0.8599256 | 0.5543369 | 0.5814908 | 0.823028 | 0.937788 | 0.6045317 |
| {Ribosomal protein 16} | 1.0252354 | 1.0438359 | 0.8948374 | 1.0386748 | 1.0029415 | 0.9793016 | 1.0856812 |
| Organic cation transporter 3 | 0.8751704 | 0.9084476 | 0.7972704 | 0.9842768 | 0.8491043 | 0.905133 | 0.9865587 |
| Calpain 2 | 1.0826476 | 1.0532597 | 0.9915702 | 1.0923312 | 1.0106864 | 0.9566407 | 1.1091263 |
| Phase-1 RCT-102 | 0.7119781 | 0.8883383 | 0.7421636 | 0.8776203 | 0.6572039 | 0.8795846 | 0.6873554 |
| Equilbrative nitrobenzylthioinosine-sensitive | 0.8032472 | 0.8632305 | 0.8469448 | 0.9200506 | 0.9450718 | 1.0165243 | 0.962967 |
| nucleoside transporter | | | | | | | |
| Phase-1 RCT-227 | 1.3250047 | 1.0611315 | 0.7923204 | 0.8958325 | 0.9708971 | 1.0614043 | 1.1983253 |
| N-hydroxy-2-acetylaminofluorene sulfotransferase (ST1C1) | 0.789873 | 0.8385057 | 0.7345083 | 0.8939316 | 0.9061073 | 0.9941402 | 1.0240856 |
| Cathepsin S | 0.7921876 | 0.9903437 | 0.8504164 | 1.193737 | 0.8814381 | 1.0617992 | 0.9584411 |
| Apolipoprotein CIII | 0.9312711 | 0.9479446 | 1.0380588 | 0.9392062 | 1.0380639 | 1.119457 | 1.0560069 |
| Cytochrome P450 2A3 | 0.7753239 | 0.9840111 | 0.8980362 | 0.8310163 | 0.8753931 | 0.998547 | 0.8584923 |
| Cytochrome P450 2C23 | 0.8993096 | 0.93078 | 0.9813502 | 1.0983789 | 0.8615699 | 1.0560472 | 1.0919918 |
| Apolipoprotein C1 | 1.0983608 | 1.0197501 | 0.7849336 | 1.1778928 | 1.1015816 | 1.2234246 | 1.0754135 |
| Betaine homocysteine methyltransferase (BHMT) | 1.5423923 | 0.9790118 | 1.4889206 | 1.2767898 | 1.0300382 | 1.0387816 | 1.1711787 |
| Paraoxonase 1 | 0.7991151 | 0.9529607 | 0.6476971 | 0.9921994 | 0.654363 | 0.8562416 | 0.7933408 |
| Phase-1 RCT-207 | 1.0975465 | 1.0098662 | 1.2988082 | 0.9494148 | 0.9802561 | 0.9743009 | 0.9584449 |
| Cathepsin B | 0.9759141 | 1.0829252 | 0.9123913 | 0.9643489 | 0.9824573 | 0.9771403 | 1.0673535 |
| Phase-1 RCT-144 | 1.0255679 | 0.9954954 | 1.0418373 | 1.149675 | 1.0478411 | 1.1515938 | 1.1044135 |
| Alpha-1 microglobulin/bikunin precursor (Ambp) | 1.0294667 | 1.00206 | 0.923935 | 1.1542581 | 1.0588146 | 1.2695646 | 1.2126223 |
| Cyclin D1 | 0.7522711 | 0.9929847 | 0.8431717 | 1.3517952 | 0.9463512 | 0.7222391 | 0.8216401 |
| Presenilin-1 | 1.0180442 | 1.1434969 | 0.831435 | 1.1203228 | 0.6226333 | 1.4842103 | 0.87424 |
| Protein tyrosine phosphatase, receptor type, D | 0.8843585 | 0.9201573 | 0.7371344 | 0.9172241 | 0.723955 | 0.8835598 | 0.9253818 |
| Multidrug resistant protein-1 | 1.3259294 | 1.3870392 | 1.5350157 | 1.242927 | 1.6336164 | 1.5589206 | 1.4011728 |
| Cathepsin L, sequence 2 | 0.9053881 | 0.9213027 | 0.8581826 | 1.1526176 | 0.8993554 | 1.0567297 | 0.8728668 |
| JNK1 stress activated protein kinase | 0.8933059 | 0.886892 | 0.813664 | 0.8354688 | 0.8540309 | 0.819115 | 0.8043814 |
| Phase-1 RCT-43 | 0.9566959 | 0.8786368 | 0.9999999 | 1.1606143 | 1.0204688 | 0.9712232 | 0.9832172 |
| Tissue plasminogen activator | 1.1876404 | 0.9475227 | 0.9619522 | 0.9811961 | 0.8445485 | 0.9399132 | 1.1014608 |
| Ubiquitin conjugating enzyme (RAD 6 | 0.8912513 | 0.8533623 | 0.815912 | 1.0105104 | 0.9614353 | 0.9149979 | 1.0526028 |
| homologue) | | | | | | | |
| Phase-1 RCT-173 | 1.1196227 | 1.0138404 | 1.0392314 | 0.9381295 | 1.0144306 | 0.812565 | 0.9279547 |
| Ornithine decarboxylase | 1.5649095 | 1.2799121 | 1.981091 | 1.632584 | 1.5947284 | 1.662608 | 1.646685 |
| Zinc finger protein | 1.0346603 | 0.9351077 | 1.071541 | 1.023906 | 0.9353743 | 0.8737694 | 1.0536126 |
| CDK108 | 1.1413649 | 1.0519322 | 0.6865063 | 1.0166698 | 1.1126 | 1.0911391 | 1.1462932 |
| Phase-1 RCT-117 | 1.0661517 | 1.0485417 | 1.0328795 | 1.2404894 | 0.6065105 | 0.8982331 | 1.222006 |
| {Phase-1 RCT 98} | 1.193153 | 1.0195495 | 1.1576043 | 1.1471956 | 0.9721188 | 1.1650281 | 1.419097 |
| Aquaporin-3 (AQP3) | 1.2830746 | 1.0527595 | 1.1751745 | 1.033078 | 1.0603076 | 0.896519 | 1.1353222 |
| Cholesterol esterase | 0.5421339 | 0.8372366 | 0.6312923 | 0.7113413 | 0.6843076 | 0.8468731 | 0.6683747 |
| Ribosomal protein S17 | 1.0060154 | 0.9923112 | 0.7355 | 1.1973672 | 0.9946008 | 1.1156799 | 1.1021272 |
| Poly(ADP-ribose) polymerase | 1.0000179 | 0.9691063 | 1.0385563 | 1.0784954 | 1.1966449 | 1.0395504 | 1.2084321 |
| Annexin V | 0.9183745 | 1.0909528 | 1.0365531 | 1.4713033 | 1.1040015 | 1.1658885 | 1.0738853 |
| Phase-1 RCT-33 | 1.0895994 | 1.0277511 | 0.9723309 | 0.94125 | 0.9355788 | 0.8652085 | 0.8880244 |
| Phase-1 RCT-61 | 0.9475589 | 0.8836913 | 1.1995426 | 0.7786248 | 0.6835477 | 0.8717371 | 0.5496894 |
| Retinol dehydrogenase type III | 0.7569451 | 0.8420706 | 0.8367764 | 0.952692 | 0.833178 | 0.9589898 | 0.8922828 |
| ATPase inhibitor (rat mitochondrial IF1 protein) | 0.690665 | 0.6780174 | 0.6066249 | 0.8450211 | 0.8053493 | 0.8664148 | 0.7559661 |
| Thymidylate synthase | 0.7341977 | 0.9221479 | 0.9468976 | 0.6729181 | 0.7838271 | 1.0523049 | 0.6963643 |
| Interleukin-18 | 0.9525725 | 0.9881909 | 1.1581508 | 0.9886591 | 0.927273 | 0.8679296 | 0.9624977 |
| Lecithin: cholesterol acyltransferase | 0.9966683 | 0.8310254 | 0.8039441 | 0.8926497 | 0.7254323 | 0.8720654 | 0.8361965 |
| Contrapsin-like protease inhibitor (CPi-21) | 1.2987833 | 0.9583395 | 0.9115253 | 0.9550632 | 0.7194831 | 0.8898906 | 1.0610173 |
| Proliferating cell nuclear antigen gene | 1.0901912 | 1.1079373 | 1.0218015 | 0.9238662 | 1.0464454 | 0.9989751 | 0.9592986 |
| Phase-1 RCT-230 | 1.0208163 | 0.996857 | 1.0705596 | 0.9985827 | 0.9618669 | 0.8800221 | 0.8699032 |
| Cytochrome P450 2D18 | 1.1797054 | 0.979142 | 1.1769613 | 1.0497086 | 1.2570934 | 1.2124425 | 1.1441336 |
| Phase-1 RCT-48 | 0.9811075 | 0.9191779 | 1.0065954 | 1.5672988 | 0.9654068 | 1.2477021 | 1.2887195 |
| Phase-1 RCT-292 | 1.0768162 | 1.0117143 | 1.0522372 | 1.2349945 | 1.0557908 | 1.055908 | 1.1430074 |
| Arginosuccinate synthetase 1 | 1.0223128 | 1.1595207 | 1.3161118 | 1.1009265 | 1.2580545 | 1.169026 | 1.333116 |
| C-reactive protein | 0.8368834 | 1.0084556 | 0.7213458 | 0.8373531 | 0.645536 | 0.8380184 | 0.7933075 |

| | DIF | DMN | DMN | DMN | DOX | DOX |
|---|---|---|---|---|---|---|
| Compound-Dose | 100 | 20 | 20 | 20 | 12 | 12 |
| Animal Number | 259 | 1757 | 1758 | 1759 | 1257 | 1258 |
| Liver Toxicity Necrosis Classification | no | yes | yes | yes | no | no |
| Gene Name | | | | | | |
| Osteoactivin | 1.0058699 | 9.442778 | 3.1861467 | 7.710055 | 1.5318965 | 1.248942 |
| Stathmin | 1.0984112 | 2.837953 | 2.026919 | 2.1916366 | 0.929551 | 0.9166068 |
| Calpactin I heavy chain | 1.2781343 | 5.946344 | 2.4033005 | 3.843169 | 1.364926 | 0.9719276 |
| Phase-1 RCT-179 | 0.8715377 | 2.1172245 | 1.565233 | 2.23797 | 0.9353874 | 0.8958505 |
| Phase-1 RCT-107 | 0.9914919 | 0.9711943 | 0.8233318 | 0.8040169 | 0.712991 | 0.8608753 |
| Heme oxygenase | 1.0824268 | 5.4685683 | 2.2985709 | 5.313715 | 1.3472687 | 1.3593642 |
| Collagen type II | 1.1746676 | 2.1576884 | 1.204181 | 2.112729 | 0.8093249 | 0.8740208 |
| Phase-1 RCT-81 | 1.3015895 | 0.8012751 | 0.8665455 | 0.7177912 | 1.0200853 | 0.9719168 |
| Glycine methyltransferase | 1.0293223 | 0.3017397 | 0.593106 | 0.3782954 | 0.6562265 | 0.8427153 |
| Cystatin C | 1.0690917 | 1.4454843 | 1.1321563 | 1.7174932 | 0.7241628 | 0.8517085 |
| Melanoma-associated antigen ME491 | 1.0946976 | 3.0472207 | 1.9462024 | 3.3786554 | 1.0031643 | 0.987742 |
| Phase-1 RCT-146 | 1.0579295 | 2.4925826 | 1.5479223 | 1.9992155 | 1.0407066 | 0.9906917 |
| Gamma-actin, cytoplasmic | 0.9544681 | 2.5131214 | 1.7987823 | 2.5319722 | 1.0071651 | 0.9962333 |
| Adenine nucleotide translocator 1 | 0.9927541 | 1.0585098 | 0.9469777 | 1.0003195 | 0.9232867 | 0.5935973 |
| Thymosin beta-10 | 0.7772115 | 4.0785317 | 2.4406278 | 4.5365753 | 1.0889648 | 1.0929667 |
| Insulin-like growth factor I, exon 6 | 0.6711259 | 0.2770893 | 0.4514781 | 0.3439581 | 0.6867304 | 0.5121124 |
| Phase-1 RCT-78 | 1.0224321 | 0.5740823 | 0.7338466 | 0.5944242 | 0.8899378 | 0.8642922 |
| Alpha-2-microglobulin | 0.5800234 | 0.0376963 | 0.0624851 | 0.0280375 | 0.1896285 | 0.1902864 |
| High affinity IgE receptor gamma chain | 0.8977481 | 2.876352 | 1.5395492 | 3.1394496 | 0.8615171 | 0.84347 |
| (FcERIgamma) | | | | | | |
| 60S ribosomal protein L6 | 1.0085357 | 1.84379 | 1.4363431 | 1.828529 | 1.4546772 | 1.2492582 |
| IgE binding protein | 1.1290866 | 7.1561027 | 2.3673766 | 6.547444 | 1.2138687 | 0.9082446 |
| Phase-1 RCT-149 | 1.1176883 | 0.8357205 | 0.7932243 | 0.8226692 | 0.8550182 | 0.6937632 |
| Cytochrome P450 2C11 | 0.9888295 | 0.0728319 | 0.2163671 | 0.1319173 | 1.4688259 | 1.6012837 |
| Uncoupling protein 2 | 0.7604113 | 4.83376 | 2.4350781 | 5.763337 | 1.0645705 | 0.886826 |
| Cofilin | 1.589044 | 2.2259853 | 1.5292068 | 2.1952417 | 0.8186942 | 0.9260877 |
| Alpha-tubulin | 0.9614276 | 3.5484726 | 3.055657 | 3.4715638 | 0.8888472 | 0.890706 |
| Stearyl-CoA desaturase, liver | 1.1621497 | 0.0598846 | 0.2231559 | 0.0524004 | 0.0699733 | 0.0868258 |
| Glutathione S-transferase theta-1 | 0.9199781 | 0.8279761 | 2.0666187 | 1.1185882 | 0.7926948 | 0.6492104 |
| alpha-1,2-fucosyltrans-ferase | 0.4941276 | 0.7317838 | 1.0423495 | 0.8160039 | 1.0172954 | 1.036631 |
| Phase-1 RCT-138 | 1.0644039 | 2.4232948 | 1.4247289 | 2.6452522 | 0.8573463 | 0.8116245 |
| Phase-1 RCT-295 | 1.0835729 | 2.2485178 | 2.0533373 | 2.2102814 | 1 | 1.1126183 |
| Phase-1 RCT-164 | 0.9022963 | 0.6127968 | 0.7069434 | 0.7253819 | 0.7667261 | 0.8091663 |
| Alpha-2-macroglobulin, sequence 2 | 0.946254 | 0.9629874 | 0.7221739 | 0.7685536 | 0.9097117 | 1.0687972 |
| Biliverdin reductase | 1.5595368 | 2.2413964 | 1.7901897 | 2.533243 | 1.0937098 | 0.8614681 |
| Phase-1 RCT-121 | 0.9003083 | 3.2825465 | 1.8550783 | 3.9615777 | 1.0192453 | 0.7505463 |
| Dynamin-1 (D100) | 0.9438933 | 0.8325777 | 0.9330828 | 0.7416195 | 0.8493086 | 1.062881 |
| Phase-1 RCT-192 | 1.0315137 | 1.8207209 | 1.5489115 | 1.9038267 | 1.4109632 | 1.0575892 |
| Fatty acid synthase | 0.8117381 | 0.2562816 | 0.4656609 | 0.2802864 | 0.3906801 | 0.5782939 |
| Phase-1 RCT-211 | 0.8807131 | 2.4721289 | 1.7273381 | 2.2252955 | 1.3506508 | 1.4771662 |
| Ribosomal protein S9 | 0.8873944 | 1.7730641 | 1.5195584 | 1.9823402 | 1.1237956 | 1.0757594 |
| NADP-dependent isocitrate dehydrogenase, | 1.0589054 | 0.6403322 | 0.7668673 | 0.6147912 | 0.8754022 | 0.7418122 |
| cytosolic | | | | | | |
| Phase-1 RCT-57 | 0.9329981 | 0.7937021 | 0.8898371 | 0.7807677 | 0.8278998 | 0.6260895 |
| Phase-1 RCT-36 | 0.8647528 | 0.6899831 | 0.8333408 | 0.706374 | 0.8021429 | 0.8281738 |
| Apolipoprotein All | 0.6406018 | 0.0861621 | 0.2828916 | 0.1111 | 0.8750536 | 1.7096019 |
| Phase-1 RCT-68 | 1.2160428 | 2.2199702 | 1.5093691 | 1.8856231 | 1.088592 | 1.1477123 |
| Multidrug resistant protein-2 | 1.0464841 | 4.971008 | 4.7017784 | 3.5781097 | 3.0060558 | 3.1137574 |
| Phase-1 RCT-39 | 0.8234453 | 1.9191295 | 1.8053133 | 2.2042093 | 0.9841842 | 1.6399776 |
| NIPK | 0.6979899 | 0.7335007 | 0.7254615 | 0.7435045 | 0.8367524 | 0.820362 |
| F1-ATPase beta subunit | 0.9180906 | 0.8694347 | 1.0896698 | 0.85655 | 0.6794436 | 0.658371 |
| Elongation factor-1 alpha | 1.0046446 | 1.2077315 | 1.1456523 | 1.3293709 | 1.214006 | 1.0436596 |
| Phase-1 RCT-24 | 1.176115 | 2.949472 | 2.8701186 | 2.830043 | 0.9846496 | 1.0138435 |
| Beta-tubulin, class I | 1.312794 | 3.7664325 | 2.8386497 | 2.6551533 | 0.6888743 | 0.6277706 |
| 14-3-3 zeta | 0.9987618 | 3.0135367 | 2.543768 | 3.075995 | 1.229505 | 1.1600542 |
| Alpha-prothymosin | 1.8258806 | 1.8180588 | 1.4252074 | 2.0055177 | 0.7175884 | 0.8772195 |
| Phase-1 RCT-58 | 0.8627407 | 1.3469259 | 1.8504773 | 1.3780502 | 2.3486412 | 1.9247121 |
| Phase-1 RCT-290 | 1.0416597 | 0.4041523 | 0.4028758 | 0.2979909 | 0.6486315 | 1.0634831 |
| Transitional endoplasmic reticulum ATPase | 0.9472297 | 0.8872732 | 0.9487317 | 0.8605495 | 0.9047477 | 0.737032 |
| Beta-actin | 0.9222284 | 4.848096 | 3.4460404 | 5.2669044 | 0.7888961 | 0.7767342 |
| Thioredoxin-1 (Trx1) | 0.7632177 | 0.9891404 | 1.168677 | 1.081048 | 0.916866 | 1.0984569 |
| Phase-1 RCT-174 | 1.2588645 | 1.4912696 | 0.8571163 | 1.0310768 | 0.7728823 | 0.7679705 |
| Beta-actin, sequence 2 | 1.2812263 | 2.584273 | 1.9560804 | 2.9687517 | 1.2891115 | 1.171628 |
| Phase-1 RCT-109 | 0.8352155 | 1.6528914 | 1.5791022 | 1.726422 | 1.4719336 | 1.3032619 |
| Phase-1 RCT-154 | 1.2767032 | 2.4118855 | 2.198919 | 2.1739683 | 1.3080821 | 1.4243027 |
| Hypoxanthine-guanine | 1.0045193 | 1.4335092 | 1.4098204 | 1.2631109 | 0.8111888 | 0.8401588 |
| phosphoribosyltransferase | | | | | | |
| Phase-1 RCT-213 | 0.8697222 | 1.1978152 | 1.1476324 | 1.0920191 | 1.1690984 | 0.9639936 |
| Phase-1 RCT-260 | 0.7514002 | 0.7105132 | 0.6683113 | 0.6234036 | 0.7536441 | 1.562 |
| Phase-1 RCT-12 | 1.2550064 | 2.1945798 | 2.014765 | 2.007208 | 0.8169473 | 0.8020723 |
| Phase-1 RCT-293 | 1.0513705 | 7.22828 | 1.740931 | 4.982032 | 1.7123023 | 1.4897474 |
| Ribosomal protein S8 | 1.0050572 | 1.7367666 | 1.473767 | 1.7991846 | 1.286051 | 1.1354396 |
| Pyruvate kinase, muscle | 1.2937896 | 5.7798767 | 1.5646151 | 4.7186656 | 1.5680916 | 1.535858 |
| Nucleoside diphosphate kinase beta isoform | 1.2661082 | 2.1336427 | 2.1390774 | 1.9797003 | 1.2546709 | 1.1288644 |
| p55CDC | 1.0946665 | 5.307082 | 1.6167485 | 1.9818262 | 2.4564807 | 6.693563 |
| Phase-1 RCT-156 | 1.1224232 | 1.1417353 | 1.2903998 | 1.3466306 | 0.8483641 | 0.8127188 |
| Ribosomal protein L13A | 0.8318169 | 1.8561428 | 1.7518731 | 1.9462317 | 1.4647999 | 1.4037677 |
| Phase-1 RCT-258 | 1.0798373 | 1.3230892 | 1.169937 | 1.2715971 | 1.0168707 | 0.8630965 |
| Insulin-like growth factor I | 0.8894334 | 0.2585332 | 0.4108391 | 0.3130661 | 0.5222152 | 0.3889825 |
| Cytochrome P450 3A1 | 0.8159744 | 0.2122163 | 0.594982 | 0.1917113 | 0.4503593 | 0.9594204 |
| {Ribosomal protein 16} | 1.0229915 | 1.6730492 | 1.3781241 | 1.6800777 | 1.2119021 | 1.1679968 |
| Organic cation transporter 3 | 0.9837327 | 1.6684073 | 1.3712819 | 1.6919669 | 1.3253903 | 1.3193959 |
| Calpain 2 | 1.0543278 | 1.9850191 | 1.3847775 | 1.87289 | 1.0613303 | 0.9503911 |
| Phase-1 RCT-102 | 0.8809788 | 0.1547927 | 0.2092237 | 0.184574 | 0.18543111 | 0.2234322 |
| Equilbrative nitrobenzylthioinosine-sensitive | 0.8037962 | 0.3632182 | 0.5289698 | 0.4068349 | 0.5398988 | 0.7848276 |
| nucleoside transporter | | | | | | |
| Phase-1 RCT-227 | 1.0302552 | 0.4033502 | 0.6366171 | 0.4312413 | 0.888321 | 0.8083926 |
| N-hydroxy-2-acetylaminofluorene sulfotransferase (ST1C1) | 0.8254803 | 0.2357116 | 0.4051067 | 0.3332047 | 0.4686131 | 0.6107768 |
| Cathepsin S | 1.1216512 | 2.361692 | 1.7216731 | 3.2793076 | 1.4111843 | 1.551224 |
| Apolipoprotein CIII | 0.9743004 | 0.3620586 | 0.4869598 | 0.3568416 | 0.820394 | 0.8392816 |
| Cytochrome P450 2A3 | 1.0716735 | 0.210114 | 0.2640289 | 0.2339256 | 0.5676882 | 0.7011159 |
| Cytochrome P450 2C23 | 1.1271523 | 0.3486784 | 0.4402862 | 0.4216227 | 0.8907613 | 0.7508113 |
| Apolipoprotein C1 | 0.7547453 | 0.1377448 | 0.1489451 | 0.1363539 | 0.6568503 | 0.5800274 |
| Betaine homocysteine methyltransferase (BHMT) | 0.9894689 | 0.1658402 | 0.1593139 | 0.0968419 | 0.5609185 | 0.9019414 |
| Paraoxonase 1 | 0.9500654 | 0.1659688 | 0.3743397 | 0.2188219 | 0.5620652 | 0.5864776 |
| Phase-1 RCT-207 | 0.8907353 | 2.3183565 | 2.5111995 | 2.3214073 | 1.8594643 | 1.2712599 |
| Cathepsin B | 0.9663672 | 1.7232041 | 1.2393876 | 1.9117498 | 1.2033004 | 1.0096905 |
| Phase-1 RCT-144 | 1.1868571 | 1.4607508 | 1.2258269 | 1.2983661 | 0.973052 | 0.9888064 |
| Alpha-1 microglobulin/bikunin precursor (Ambp) | 1.3009173 | 0.4799612 | 0.6596317 | 0.4565953 | 1.0351863 | 0.917106 |
| Cyclin D1 | 0.9942908 | 2.126251 | 1.5951105 | 1.6715975 | 0.7147006 | 0.6271998 |
| Presenilin-1 | 1.08965 | 0.2225577 | 0.3507521 | 0.2405811 | 0.28987 | 0.273227 |
| Protein tyrosine phosphatase, receptor type, D | 1.0069262 | 0.3424624 | 0.3974167 | 0.3542754 | 1.5584623 | 1.1290823 |
| Multidrug resistant protein-1 | 1.2439513 | 6.283614 | 4.383023 | 3.7162 | 2.988639 | 2.4482975 |
| Cathepsin L, sequence 2 | 1.226659 | 1.5442618 | 1.2564187 | 1.4532796 | 1.9465402 | 2.1448183 |
| JNK1 stress activated protein kinase | 0.7580049 | 0.5496806 | 0.6447266 | 0.5406055 | 1.1475723 | 1.4773842 |
| Phase-1 RCT-43 | 1.0984939 | 1.2377932 | 1.3049309 | 1.3950635 | 0.7646852 | 0.7672727 |
| Tissue plasminogen activator | 1.166615 | 1.6605104 | 1.2598134 | 1.4500462 | 0.7126551 | 0.6707375 |
| Ubiquitin conjugating enzyme (RAD 6 | 0.9747115 | 1.4286754 | 1.3170702 | 1.6026064 | 0.9970807 | 0.9748122 |
| homologue) | | | | | | |
| Phase-1 RCT-173 | 0.9297075 | 1.2639678 | 1.0804424 | 1.2427564 | 0.9463901 | 0.8621078 |
| Ornithine decarboxylase | 1.5781375 | 3.7153418 | 2.5020714 | 2.6578214 | 1.0285732 | 1.007474 |
| Zinc finger protein | 1.1113871 | 1.7510642 | 1.2373524 | 1.6320338 | 1.0651618 | 0.6165031 |
| CDK108 | 1.1048334 | 0.837883 | 0.7621119 | 0.7115791 | 0.8294181 | 0.8326395 |
| Phase-1 RCT-117 | 0.8519294 | 0.7504737 | 0.8040006 | 0.5911028 | 1.117608 | 1.3538092 |
| {Phase-1 RCT 98} | 1.2916908 | 0.8875126 | 0.8839839 | 0.7908146 | 0.7311078 | 0.7195701 |
| Aquaporin-3 (AQP3) | 0.9500983 | 0.775085 | 0.8545302 | 0.7656481 | 0.8706986 | 0.9364509 |
| Cholesterol esterase | 0.9344577 | 0.1390794 | 0.4223203 | 0.1788979 | 0.7335409 | 0.5145294 |
| Ribosomal protein S17 | 1.1746975 | 1.3928349 | 1.122626 | 1.3569356 | 1.1736933 | 1.2171795 |
| Poly(ADP-ribose) polymerase | 1.0491456 | 1.4426783 | 1.3661331 | 1.5267507 | 0.8588876 | 0.7631447 |
| Annexin V | 1.2149216 | 2.5433059 | 1.3920951 | 2.390962 | 1.0785352 | 0.8450508 |
| Phase-1 RCT-33 | 0.835891 | 0.4265149 | 0.7076982 | 0.497881 | 0.7538697 | 0.7784027 |
| Phase-1 RCT-61 | 0.8849878 | 1.2180402 | 1.1296659 | 1.2059668 | 1.0763998 | 1.1316828 |
| Retinol dehydrogenase type III | 0.9125143 | 0.3628763 | 0.6706508 | 0.4357715 | 0.7634023 | 0.8364956 |
| ATPase inhibitor (rat mitochondrial IF1 protein) | 0.8419712 | 0.1206003 | 0.2123534 | 0.1401303 | 0.653209 | 0.6702426 |
| Thymidylate synthase | 1.1248381 | 1.5835414 | 1.2519994 | 1.391637 | 1.3240148 | 1.0739396 |
| Interleukin-18 | 0.9787818 | 1.3184268 | 1.078615 | 1.501565 | 1.2137866 | 0.8310626 |
| Lecithin: cholesterol acyltransferase | 0.8720303 | 0.5684852 | 0.8010593 | 0.611793 | 1.3640891 | 1.3468955 |
| Contrapsin-like protease inhibitor (CPi-21) | 1.1877922 | 0.1883951 | 0.2515224 | 0.2760025 | 0.2782638 | 0.2218148 |
| Proliferating cell nuclear antigen gene | 0.7986177 | 2.4221308 | 1.555596 | 2.1440887 | 0.9063702 | 0.5901841 |
| Phase-1 RCT-230 | 0.7868269 | 1.9285115 | 1.4944279 | 2.4991176 | 0.8568987 | 1.8206348 |
| Cytochrome P450 2D18 | 0.9410191 | 0.3934356 | 0.6027201 | 0.4152303 | 0.9145611 | 0.6208303 |
| Phase-1 RCT-48 | 1.2881846 | 0.3941817 | 0.5063516 | 0.4845119 | 0.7619011 | 0.8030474 |
| Phase-1 RCT-292 | 1.0587884 | 0.7484225 | 0.8687803 | 0.8358251 | 1.0213639 | 0.9972017 |
| Arginosuccinate synthetase 1 | 1.2072364 | 0.351914 | 0.3814044 | 0.2738428 | 0.6420031 | 0.7132765 |
| C-reactive protein | 0.9822225 | 0.4259598 | 0.5157487 | 0.4644139 | 1.6803964 | 1.2893289 |

| | ERY | ERY | ERY | ERY | ERY | ERY | EST |
|---|---|---|---|---|---|---|---|
| Compound-Dose | 40 | 40 | 40 | 160 | 160 | 160 | 0.1 |
| Animal Number | 347 | 348 | 349 | 357 | 358 | 359 | 1427 |
| Liver Toxicity Necrosis Classification | no | no | no | no | no | no | no |
| Gene Name | | | | | | | |
| Osteoactivin | 1.2026261 | 1.3092251 | 1.5923856 | 1.356613 | 1.386012 | 1.6869136 | 1.3028886 |
| Stathmin | 0.7425447 | 0.7586127 | 0.7041455 | 0.6314703 | 0.855919 | 0.7748793 | 0.9223254 |
| Calpactin I heavy chain | 0.7680881 | 0.9686016 | 0.8263257 | 1.1213621 | 0.8167563 | 0.8342212 | 0.9375908 |
| Phase-1 RCT-179 | 1.2585251 | 0.7787904 | 0.6853576 | 0.6042856 | 1.1812758 | 0.694269 | 1.2012448 |
| Phase-1 RCT-107 | 0.7147028 | 0.8178533 | 0.7827825 | 1.1586772 | 0.7517585 | 0.7673632 | 0.7569441 |
| Heme oxygenase | 1.2488883 | 1.1877031 | 2.2281592 | 1.5267172 | 1.0935316 | 1.35694 | 0.6973686 |
| Collagen type II | 0.3924841 | 0.5576515 | 0.5752401 | 0.6646158 | 0.7270265 | 0.7585186 | 0.8895812 |
| Phase-1 RCT-81 | 1.1517357 | 0.7662529 | 1.0327783 | 0.9474717 | 0.9932416 | 1.4390533 | 1.3133014 |
| Glycine methyltransferase | 0.674755 | 0.8669856 | 0.8228823 | 1.2389716 | 0.7620661 | 0.7551005 | 0.8259236 |
| Cystatin C | 1.6805317 | 1.6770654 | 1.9283756 | 1.7253051 | 1.916018 | 1.5475 | 1.2361157 |
| Melanoma-associated antigen ME491 | 0.8697023 | 0.7901702 | 1.1067545 | 1.3265237 | 0.9917831 | 1.1685394 | 1.029649 |
| Phase-1 RCT-146 | 0.6504979 | 0.6889551 | 0.6658551 | 0.6494533 | 0.8574185 | 0.7677939 | 0.8269724 |
| Gamma-actin, cytoplasmic | 0.7284979 | 0.7987466 | 0.9145621 | 1.2765194 | 1.0076146 | 1.112042 | 1.1267369 |
| Adenine nucleotide translocator 1 | 0.6938838 | 0.7095989 | 0.5970422 | 0.6428037 | 0.7402954 | 0.6565518 | 1.0655949 |
| Thymosin beta-10 | 1.2468116 | 1.0271078 | 1.1870034 | 1.2066203 | 1.4952633 | 1.3744348 | 1.6849355 |
| Insulin-like growth factor I, exon 6 | 1.7878871 | 2.9814765 | 1.7998725 | 2.3924198 | 1.6943586 | 1.2290379 | 0.722779 |
| Phase-1 RCT-78 | 1.2980974 | 1.1450413 | 1.2494638 | 1.1896417 | 0.9948611 | 1.10499 | 1.0022836 |
| Alpha-2-microglobulin | 1.023775 | 1.1298805 | 1.1925472 | 0.7855749 | 0.8935513 | 0.9363607 | 1.0818118 |
| High affinity IgE receptor gamma chain | 0.9549483 | 1.0581539 | 1.0802021 | 1.133739 | 0.9506681 | 1.1409091 | 1.3028888 |
| (FcERIgamma) | | | | | | | |
| 60S ribosomal protein L6 | 1.1445643 | 1.1708345 | 0.7669652 | 0.9833487 | 1.1787152 | 1.265643 | 1.3805608 |
| IgE binding protein | 1.0976462 | 1.2172916 | 1.1906631 | 1.2907429 | 1.1248055 | 1.2270533 | 1.1617582 |
| Phase-1 RCT-149 | 0.9581749 | 0.8636389 | 0.9420309 | 0.8345354 | 0.9667115 | 1.0363637 | 0.8583695 |
| Cytochrome P450 2C11 | 1.2974398 | 1.3428787 | 1.4160886 | 1.2898746 | 1.4803143 | 1.8693297 | 0.8692585 |
| Uncoupling protein 2 | 0.8197387 | 0.7520121 | 0.8110218 | 0.9060019 | 0.9007907 | 1.0503529 | 1.2120478 |
| Cofilin | 1.1596153 | 1.1228223 | 1.0971475 | 0.969414 | 1.1017263 | 1.1627706 | 1.5553347 |
| Alpha-tubulin | 0.7245302 | 1 | 0.7313014 | 1.0490707 | 0.9306842 | 0.822619 | 1.0797734 |
| Stearyl-CoA desaturase, liver | 2.4862251 | 2.1931744 | 0.8620646 | 1.2563969 | 2.0054743 | 0.9742059 | 0.4725772 |
| Glutathione S-transferase theta-1 | 0.9295537 | 1.0754044 | 0.8975942 | 0.6840228 | 0.9194123 | 0.8708334 | 1.4352432 |
| alpha-1,2-fucosyltrans-ferase | 1.9225607 | 2.1253161 | 1.9157763 | 1.5668225 | 2.0981205 | 2.0072074 | 1.4039922 |
| Phase-1 RCT-138 | 1.1647573 | 1.4845192 | 1.3328382 | 1.5551229 | 1.2897491 | 1.0851772 | 1.1232793 |
| Phase-1 RCT-295 | 1.1677265 | 1.1612668 | 1.2289724 | 1.2162092 | 1.2209884 | 1.2437774 | 1.2704895 |
| Phase-1 RCT-164 | 1.0402565 | 0.8010564 | 0.8734193 | 0.7082773 | 0.7169674 | 0.6348221 | 1.0848061 |
| Alpha-2-macroglobulin, sequence 2 | 1.1449685 | 1.1407714 | 1.1071364 | 1.2010041 | 1.0869308 | 1.1296433 | 1.1355637 |
| Biliverdin reductase | 0.6681442 | 0.8702079 | 0.7821181 | 0.9126887 | 0.8095307 | 0.902802 | 1.2259324 |
| Phase-1 RCT-121 | 0.6119899 | 0.604617 | 0.5377492 | 0.4841671 | 0.7870929 | 0.5461627 | 0.8527967 |
| Dynamin-1 (D100) | 1.8601369 | 2.0465214 | 1.6911687 | 1.9973686 | 1.9360586 | 1.5303355 | 1.1959385 |
| Phase-1 RCT-192 | 1.0185039 | 1.0173885 | 1.0090277 | 0.9100355 | 1.1424866 | 1.0206897 | 1.3901187 |
| Fatty acid synthase | 1.5265402 | 2.2092402 | 0.7746813 | 1.9082855 | 2.147269 | 0.9009652 | 0.8563778 |
| Phase-1 RCT-211 | 1.0838871 | 1.4267083 | 1.8128481 | 1.1846884 | 1.1920456 | 1.1102041 | 0.9257974 |
| Ribosomal protein S9 | 0.9792407 | 1.0527427 | 1.0238551 | 1.0059303 | 0.93758 | 1.2659887 | 1.6698213 |
| NADP-dependent isocitrate dehydrogenase, | 1.4509869 | 1.2861049 | 1.307364 | 1.3523717 | 1.4610555 | 1.136542 | 1.1863923 |
| cytosolic | | | | | | | |
| Phase-1 RCT-57 | 0.6953401 | 0.9764028 | 0.6316779 | 0.6107485 | 0.6034507 | 0.5506203 | 0.9563354 |
| Phase-1 RCT-36 | 1.3508853 | 1.7068022 | 1.080213 | 1.4185789 | 1.5688632 | 1.1356467 | 0.9007497 |
| Apolipoprotein All | 1.9483172 | 1.1647887 | 0.818787 | 1.3378301 | 2.128592 | 0.8443484 | 1.2570814 |
| Phase-1 RCT-68 | 0.9644307 | 1.0135814 | 0.9458378 | 1.0007375 | 0.8867373 | 0.9091954 | 0.8880238 |
| Multidrug resistant protein-2 | 1.0429567 | 1.0760014 | 1.4257512 | 1.1277463 | 1.2429858 | 1.3760002 | 0.7774884 |
| Phase-1 RCT-39 | 0.7656649 | 0.8741686 | 1.0414276 | 0.7266601 | 0.5917676 | 0.7445009 | 0.5532815 |
| NIPK | 1.1321809 | 1.0811802 | 1.2216712 | 1.297203 | 1.2976403 | 1.1025965 | 0.7136357 |
| F1-ATPase beta subunit | 1.0151451 | 0.9459272 | 1.1287054 | 1.323386 | 1.1881921 | 1.6497014 | 1.2688036 |
| Elongation factor-1 alpha | 1.2920802 | 1.1862699 | 1.2783417 | 1.8031505 | 1.1735505 | 1.4266558 | 1.2617097 |
| Phase-1 RCT-24 | 0.6776514 | 0.6480965 | 0.6620824 | 0.687458 | 0.7537618 | 0.7055772 | 1.0362625 |
| Beta-tubulin, class I | 0.8379166 | 1.0013204 | 0.8198057 | 1.0521299 | 0.8478207 | 0.8179373 | 0.8588983 |
| 14-3-3 zeta | 0.736935 | 0.7811954 | 0.6178175 | 0.5725071 | 0.7398078 | 0.6376297 | 0.7012075 |
| Alpha-prothymosin | 1.2102606 | 1.5774446 | 1.1910688 | 0.9513859 | 1.0076987 | 0.7675907 | 1.2798495 |
| Phase-1 RCT-58 | 2.1416273 | 0.9487481 | 0.6084607 | 1.6120844 | 1.434915 | 0.569697 | 0.4393534 |
| Phase-1 RCT-290 | 1.3146665 | 2.3442922 | 1.6826447 | 2.029261 | 2.4804437 | 1.4408379 | 0.7443401 |
| Transitional endoplasmic reticulum ATPase | 0.7920154 | 0.620732 | 0.7804446 | 0.7408332 | 0.7511069 | 0.8645741 | 1.0200297 |
| Beta-actin | 0.572538 | 0.6642112 | 0.4876743 | 0.5900502 | 0.5154992 | 0.4382124 | 0.8552747 |
| Thioredoxin-1 (Trx1) | 1.7396363 | 2.049625 | 1.9872942 | 2.1548986 | 1.9985057 | 1.6469182 | 1.2253783 |
| Phase-1 RCT-174 | 0.8654782 | 0.9344669 | 0.8463278 | 0.8049887 | 0.7538258 | 0.9803758 | 1.2957604 |
| Beta-actin, sequence 2 | 0.9035499 | 1.6599369 | 0.7832511 | 1.7396603 | 0.9737464 | 0.7929266 | 1.1995757 |
| Phase-1 RCT-109 | 1.277373 | 1.558861 | 1.5187172 | 1.3404197 | 1.4169472 | 1.2594873 | 1.5474713 |
| Phase-1 RCT-154 | 1.041192 | 1.0838377 | 0.8929721 | 1.0017782 | 1.0443689 | 0.8677966 | 1.0425779 |
| Hypoxanthine-guanine | 0.6990942 | 0.8591362 | 0.3990998 | 0.4279831 | 0.5440439 | 0.2899934 | 0.9881675 |
| phosphoribosyltransferase | | | | | | | |
| Phase-1 RCT-213 | 0.7764919 | 0.9132763 | 0.7539966 | 0.6553537 | 0.7836958 | 0.6918587 | 0.9256873 |
| Phase-1 RCT-260 | 0.7185516 | 0.8969128 | 0.9157651 | 0.877345 | 0.5832552 | 0.7792881 | 0.5363454 |
| Phase-1 RCT-12 | 0.8524395 | 0.8968348 | 0.7343001 | 0.9741783 | 0.8257312 | 0.8391931 | 0.7299666 |
| Phase-1 RCT-293 | 1.7012845 | 1.5405062 | 1.5333056 | 2.0358284 | 1.50766 | 1.5779864 | 1.2358888 |
| Ribosomal protein S8 | 1.6027735 | 2.0595481 | 2.0284984 | 2.3672738 | 1.881327 | 1.9100357 | 1.7117964 |
| Pyruvate kinase, muscle | 0.7849323 | 0.915493 | 0.8613622 | 1.3102185 | 0.9287162 | 1.139394 | 0.7015421 |
| Nucleoside diphosphate kinase beta isoform | 1.0080771 | 1.2025845 | 1.1355151 | 1.2897794 | 1.1927335 | 1.0598292 | 1.4654111 |
| p55CDC | 0.5657392 | 0.6866197 | 0.6584074 | 0.7700139 | 0.7952437 | 1.4647888 | 1.1938545 |
| Phase-1 RCT-156 | 0.9440913 | 0.9587625 | 1.0458912 | 0.9524584 | 0.9267878 | 1.0501037 | 0.8441618 |
| Ribosomal protein L13A | 1.6738642 | 2.0729465 | 1.6548003 | 1.6627603 | 1.8797423 | 1.5175807 | 1.5759262 |
| Phase-1 RCT-258 | 0.8573057 | 0.8998435 | 0.8093548 | 0.8538491 | 0.8544779 | 0.8274144 | 0.9045451 |
| Insulin-like growth factor I | 1.6269194 | 1.9537417 | 2.182228 | 1.7029971 | 1.8362707 | 1.4235052 | 1.0343982 |
| Cytochrome P450 3A1 | 1.373849 | 1.3614502 | 1.5493499 | 1.1007757 | 1.0566419 | 1.6822007 | 1.873922 |
| {Ribosomal protein 16} | 1.4650297 | 1.3823649 | 1.3384824 | 1.5932895 | 1.5649108 | 1.7208945 | 1.3337609 |
| Organic cation transporter 3 | 1.1471851 | 1.0697277 | 0.7956198 | 1.063088 | 1.1848621 | 1.2040404 | 1.4627944 |
| Calpain 2 | 0.9670508 | 1.0259094 | 1.0658182 | 1.2313174 | 1.0374169 | 1.2190478 | 1.0319394 |
| Phase-1 RCT-102 | 1.078858 | 0.7399494 | 0.6102607 | 0.7509714 | 1.0449277 | 0.664151 | 0.9876862 |
| Equilbrative nitrobenzylthioinosine-sensitive | 0.832004 | 0.8856723 | 1.0420256 | 1.1355275 | 0.677312 | 0.9167568 | 0.7833646 |
| nucleoside transporter | | | | | | | |
| Phase-1 RCT-227 | 1.3546549 | 0.9885967 | 1.1172982 | 1.2014492 | 0.8836261 | 1.2844068 | 1.2234123 |
| N-hydroxy-2-acetylaminofluorene sulfotransferase (ST1C1) | 1.0899092 | 1.1319861 | 1.4620328 | 1.4302275 | 1.0069444 | 1.5106229 | 1.0805657 |
| Cathepsin S | 1.2370715 | 1.4700941 | 1.9400831 | 1.8396395 | 1.4636822 | 1.5979567 | 1.547967 |
| Apolipoprotein CIII | 1.0044744 | 0.9434446 | 1.0640476 | 0.6288675 | 0.9739689 | 0.9489717 | 1.4994137 |
| Cytochrome P450 2A3 | 1.5227032 | 1.1715482 | 1.5994604 | 0.8860817 | 1.5536392 | 1.1439302 | 0.6331493 |
| Cytochrome P450 2C23 | 1.8157617 | 1.262301 | 1.0541415 | 1.1111095 | 1.3305496 | 0.7714885 | 1.3326943 |
| Apolipoprotein C1 | 1.2261835 | 1.1967651 | 1.1390938 | 0.8270922 | 1.0211895 | 1.4266938 | 1.6569026 |
| Betaine homocysteine methyltransferase (BHMT) | 1.6409509 | 3.3976142 | 2.1532602 | 2.3424537 | 3.284086 | 1.5356154 | 0.8192176 |
| Paraoxonase 1 | 1.064868 | 0.7929918 | 1.3002454 | 1.1449146 | 1.1623605 | 2.056179 | 1.3656 |
| Phase-1 RCT-207 | 0.7381812 | 0.7629108 | 0.626528 | 0.5377607 | 0.7942925 | 0.5595092 | 0.8876154 |
| Cathepsin B | 2.007732 | 1.5547558 | 1.8220023 | 2.183344 | 1.7193081 | 2.7044382 | 1.4004478 |
| Phase-1 RCT-144 | 0.9012885 | 0.7811188 | 0.9629155 | 0.876556 | 0.8949319 | 0.8341335 | 0.9636372 |
| Alpha-1 microglobulin/bikunin precursor (Ambp) | 1.1606231 | 0.7863564 | 1.0794332 | 0.8772971 | 0.9527208 | 1.5145804 | 1.5361983 |
| Cyclin D1 | 1.0421472 | 0.8534455 | 1.4269822 | 0.8978998 | 1.0097045 | 1.3570201 | 1.0821905 |
| Presenilin-1 | 1.4585278 | 1.0271826 | 1.3516293 | 1.1765184 | 1.6732337 | 1.8200772 | 1.2753799 |
| Protein tyrosine phosphatase, receptor type, D | 1.857611 | 2.0188892 | 2.0887713 | 2.0149822 | 1.6254431 | 1.5690396 | 1.3448243 |
| Multidrug resistant protein-1 | 0.8796588 | 1.0413111 | 1.1247864 | 1.0649937 | 1.037828 | 1.0692708 | 0.7954047 |
| Cathepsin L, sequence 2 | 1.0600194 | 1.1054034 | 1.2894096 | 1.2931957 | 0.6835779 | 1.0724325 | 1.2081088 |
| JNK1 stress activated protein kinase | 1.3753843 | 1.5661849 | 1.7443573 | 1.2649729 | 1.3372654 | 1.5757575 | 1.3314325 |
| Phase-1 RCT-43 | 0.6577716 | 0.6252622 | 0.5858333 | 0.5368241 | 0.719569 | 0.562963 | 0.8379348 |
| Tissue plasminogen activator | 1.0143508 | 1.1218393 | 1.1211767 | 1.1750767 | 0.9425674 | 1.0918919 | 0.8764754 |
| Ubiquitin conjugating enzyme (RAD 6 | 1.0642821 | 1.075513 | 0.8963088 | 0.9618935 | 1.017154 | 1.317445 | 1.6058581 |
| homologue) | | | | | | | |
| Phase-1 RCT-173 | 0.6137975 | 0.8823458 | 0.6016041 | 0.620755 | 0.6461554 | 0.5180224 | 1.1816896 |
| Ornithine decarboxylase | 0.3791591 | 0.6706306 | 0.4529491 | 0.3236948 | 0.2759356 | 0.2511723 | 0.7531398 |
| Zinc finger protein | 0.7537276 | 0.8624494 | 0.6547982 | 0.8201 | 0.8722005 | 0.5686067 | 0.9190999 |
| CDK108 | 0.7284469 | 0.8133175 | 0.8168041 | 0.9759437 | 0.793772 | 0.9112475 | 0.8758365 |
| Phase-1 RCT-117 | 1.6031692 | 1.480047 | 1.4980695 | 1.6953723 | 1.8529458 | 1.5630363 | 0.9754423 |
| {Phase-1 RCT 98} | 1.0453062 | 0.9660816 | 1.0710105 | 0.8981596 | 0.9837602 | 1.1143131 | 0.9730949 |
| Aquaporin-3 (AQP3) | 0.9245898 | 0.8876571 | 0.8217716 | 0.8768141 | 0.8034213 | 0.8023599 | 0.8174987 |
| Cholesterol esterase | 1.1637725 | 2.023266 | 1.7503668 | 1.9304663 | 1.1211764 | 0.9278716 | 1.8611549 |
| Ribosomal protein S17 | 1.3758495 | 1.2561696 | 1.4631997 | 1.5201687 | 1.3182693 | 1.7475662 | 1.6819936 |
| Poly(ADP-ribose) polymerase | 0.8592435 | 1.0418109 | 0.7395945 | 0.7682885 | 0.9291368 | 0.6722808 | 1.0269971 |
| Annexin V | 0.8939436 | 0.9338028 | 1.1057111 | 1.1032343 | 1.4242548 | 1.2257704 | 1.0582187 |
| Phase-1 RCT-33 | 2.0970068 | 2.9340434 | 1.6580931 | 1.9666188 | 2.0105865 | 1.4192469 | 1.0410335 |
| Phase-1 RCT-61 | 1.107365 | 0.842182 | 0.6711135 | 0.9108878 | 0.9640402 | 0.6222223 | 0.6911332 |
| Retinol dehydrogenase type III | 1.2840405 | 1.2224274 | 0.874509 | 0.5587774 | 0.8639023 | 0.9489317 | 1.411922 |
| ATPase inhibitor (rat mitochondrial IF1 protein) | 2.0990407 | 1.9931873 | 2.074275 | 1.6107311 | 2.0398743 | 1.1829787 | 1.3770641 |
| Thymidylate synthase | 0.6809977 | 0.8612576 | 0.6693229 | 0.7738677 | 0.8638361 | 0.8082964 | 0.8315975 |
| Interleukin-18 | 0.6230044 | 0.6391955 | 0.6055913 | 0.6748419 | 0.8568649 | 0.6629283 | 0.9329247 |
| Lecithin: cholesterol acyltransferase | 1.3880692 | 1.7109964 | 1.6754762 | 1.3115153 | 1.3805685 | 1.2966706 | 1.2860893 |
| Contrapsin-like protease inhibitor (CPi-21) | 1.3734628 | 1.690833 | 1.8650442 | 1.1007278 | 1.0546604 | 1.273477 | 1.4321889 |
| Proliferating cell nuclear antigen gene | 1.0375707 | 0.9836679 | 1.0484169 | 0.9880341 | 1.032314 | 1.2451614 | 1.1090945 |
| Phase-1 RCT-230 | 0.7296543 | 0.768562 | 0.8964782 | 0.813264 | 0.6939495 | 0.8484849 | 0.5712763 |
| Cytochrome P450 2D18 | 0.6623229 | 1.2398086 | 0.6985381 | 0.7342714 | 0.5818508 | 0.6165528 | 1.0085326 |
| Phase-1 RCT-48 | 0.8894033 | 0.9134955 | 0.8393309 | 0.8155248 | 0.8217123 | 0.7146497 | 1.1194041 |
| Phase-1 RCT-292 | 1.0427998 | 1.0942804 | 1.0219195 | 1.0739955 | 1.104166 | 0.8574238 | 0.9292005 |
| Arginosuccinate synthetase 1 | 2.0111036 | 2.7664578 | 2.2556689 | 1.7358116 | 2.163167 | 1.1585284 | 0.9233707 |
| C-reactive protein | 2.1911695 | 2.8396351 | 2.6113365 | 2.3442614 | 1.9922699 | 1.8620551 | 1.3437426 |

| | EST | EST | EST | EST | EST | ETH |
|---|---|---|---|---|---|---|
| Compound-Dose | 0.1 | 0.1 | 0.4 | 0.4 | 0.4 | 2500 |
| Animal Number | 1428 | 1429 | 1437 | 1438 | 1439 | 137 |
| Liver Toxicity Necrosis Classification | no | no | no | no | no | no |
| Gene Name | | | | | | |
| Osteoactivin | 1.0782775 | 1.2190565 | 1.3259518 | 1.1174128 | 1.4339525 | 0.80046 |
| Stathmin | 0.8174872 | 0.7248855 | 0.6194228 | 0.7569202 | 0.6022874 | 1.1454337 |
| Calpactin I heavy chain | 0.880964 | 0.8018713 | 1.1343472 | 1.1673856 | 1.0913098 | 1.0538325 |
| Phase-1 RCT-179 | 1.0557821 | 1.1326886 | 0.9900041 | 1.1454169 | 1.1209916 | 0.8158805 |
| Phase-1 RCT-107 | 0.9090909 | 1.1717374 | 0.9054694 | 1.0693711 | 0.903572 | 1.0041511 |
| Heme oxygenase | 0.9226379 | 0.7479414 | 0.8862444 | 0.8458195 | 0.4089931 | 1.0996978 |
| Collagen type II | 1.0807562 | 0.8649145 | 0.7639762 | 1.0137388 | 0.8171601 | 1.5271381 |
| Phase-1 RCT-81 | 1.266943 | 1.4841641 | 1.6579449 | 1.5810478 | 1.6705412 | 0.8580046 |
| Glycine methyltransferase | 1.3130367 | 1.483147 | 0.968223 | 1.1531949 | 1.1786005 | 0.9639936 |
| Cystatin C | 1.396885 | 0.9597118 | 1.1222692 | 1.1230419 | 0.8241904 | 0.9808163 |
| Melanoma-associated antigen ME491 | 0.8884646 | 0.7809829 | 1.2236555 | 1.0477746 | 1.1466814 | 1.1458356 |
| Phase-1 RCT-146 | 0.7957894 | 0.694517 | 0.7998275 | 0.9003861 | 0.6775733 | 1.2148983 |
| Gamma-actin, cytoplasmic | 1.1161395 | 0.7870647 | 1.1789495 | 0.9908025 | 1.1409789 | 0.8717728 |
| Adenine nucleotide translocator 1 | 1.146133 | 1.24066 | 0.5910414 | 0.9102788 | 0.847212 | 0.8577268 |
| Thymosin beta-10 | 1.5198892 | 1.2637242 | 1.5081294 | 1.4769572 | 1.2356988 | 1.1416487 |
| Insulin-like growth factor I, exon 6 | 0.9726941 | 0.9955737 | 0.8551628 | 0.8654805 | 0.9458267 | 0.9992795 |
| Phase-1 RCT-78 | 1.0385377 | 1.1237543 | 1.0074555 | 1.0235473 | 1.0491165 | 0.9764101 |
| Alpha-2-microglobulin | 1.126552 | 1.0873271 | 1.208901 | 1.2674383 | 1.0375416 | 0.6298463 |
| High affinity IgE receptor gamma chain | 1.1144354 | 0.9924606 | 1.5275712 | 1.2528139 | 1.5065242 | 0.9851973 |
| (FcERIgamma) | | | | | | |
| 60S ribosomal protein L6 | 0.9922867 | 1.185912 | 1.1907148 | 1.3128433 | 1.0231853 | 0.9444673 |
| IgE binding protein | 0.9267734 | 0.7952948 | 0.9481859 | 0.938811 | 0.8635204 | 0.9477004 |
| Phase-1 RCT-149 | 0.8699843 | 0.8418139 | 1.1328065 | 1.0499785 | 1.3419611 | 1.0516189 |
| Cytochrome P450 2C11 | 0.9180524 | 0.7856527 | 0.8279645 | 0.8506768 | 0.9454064 | 1.6300285 |
| Uncoupling protein 2 | 1.0491365 | 0.9461098 | 0.8045148 | 0.9155442 | 0.7690643 | 1.2184545 |
| Cofilin | 1.4116416 | 1.3882216 | 1.1873811 | 1.2281255 | 1.5273056 | 0.84662 |
| Alpha-tubulin | 1.0680238 | 1.0823735 | 1.000373 | 1.1894689 | 0.8342411 | 0.9572169 |
| Stearyl-CoA desaturase, liver | 1.210833 | 0.8422717 | 1.2732589 | 0.623611 | 1.4199067 | 0.8491228 |
| Glutathione S-transferase theta-1 | 1.2442105 | 1.1178256 | 0.8538747 | 1.44454 | 1.1718204 | 0.933528 |
| alpha-1,2-fucosyltrans-ferase | 1.3233782 | 1.2479471 | 1.0669714 | 0.963683 | 1.0493133 | 1.2450778 |
| Phase-1 RCT-138 | 1.0904814 | 1.0458016 | 1.1775315 | 1.0193714 | 1.1157991 | 0.9926767 |
| Phase-1 RCT-295 | 1.1392405 | 1.1143041 | 1.0764836 | 0.9391141 | 0.9489988 | 1.0628861 |
| Phase-1 RCT-164 | 1.1119642 | 1.1638545 | 0.6788201 | 0.9927115 | 0.9507861 | 1.3892102 |
| Alpha-2-macroglobulin, sequence 2 | 1.1544725 | 1.1287512 | 1.0237825 | 0.8732068 | 0.9958679 | 0.813369 |
| Biliverdin reductase | 0.9709348 | 0.8318418 | 0.7742173 | 0.8120478 | 0.814332 | 1.2445357 |
| Phase-1 RCT-121 | 0.8368421 | 0.8285904 | 0.6994574 | 0.8122291 | 0.7490429 | 0.8700905 |
| Dynamin-1 (D100) | 1.0894736 | 1.1585805 | 1.2329221 | 1.2848136 | 1.3384109 | 0.8814669 |
| Phase-1 RCT-192 | 1.0070938 | 1.247026 | 1.517279 | 1.2719655 | 1.3458884 | 0.8588728 |
| Fatty acid synthase | 1.8709335 | 1.538704 | 1.9389284 | 0.9363587 | 2.1726975 | 0.9596937 |
| Phase-1 RCT-211 | 0.8534144 | 0.8583181 | 1.2349023 | 1.1421413 | 1.4086732 | 1.0357364 |
| Ribosomal protein S9 | 1.233773 | 1.0630969 | 1.2335374 | 1.6967162 | 1.3010687 | 1.052258 |
| NADP-dependent isocitrate dehydrogenase, | 1.3212786 | 1.336419 | 1.231166 | 1.1699276 | 1.2334535 | 1.0799384 |
| cytosolic | | | | | | |
| Phase-1 RCT-57 | 0.6834895 | 0.7712978 | 0.8246235 | 1.0605625 | 0.9441226 | 0.7437168 |
| Phase-1 RCT-36 | 0.9064921 | 0.8746573 | 0.8531816 | 0.7433198 | 0.8199108 | 1.0563068 |
| Apolipoprotein All | 1.9218968 | 1.5032048 | 1.0243795 | 1.4723485 | 1.3779899 | 0.9086191 |
| Phase-1 RCT-68 | 0.8516746 | 0.8755123 | 0.9903824 | 0.9617082 | 1.0382708 | 0.9321627 |
| Multidrug resistant protein-2 | 0.9666043 | 0.6769194 | 0.6528803 | 0.7378322 | 0.9209978 | 1.3991171 |
| Phase-1 RCT-39 | 0.8474641 | 0.6807369 | 0.8655539 | 0.8689601 | 0.6253865 | 1.0859538 |
| NIPK | 0.8366945 | 0.7220435 | 0.911975 | 0.8472622 | 0.759741 | 1.0403616 |
| F1-ATPase beta subunit | 1.5200603 | 1.4477793 | 1.541793 | 1.3932399 | 1.5996294 | 1.031806 |
| Elongation factor-1 alpha | 1.5642035 | 1.2609599 | 1.3589288 | 1.4591908 | 1.5840279 | 0.8980629 |
| Phase-1 RCT-24 | 1.1052632 | 0.9317624 | 1.1035722 | 0.9775195 | 1.0593603 | 0.9324797 |
| Beta-tubulin, class I | 0.9814347 | 0.8096638 | 0.9723935 | 0.8673154 | 0.927899 | 0.8361117 |
| 14-3-3 zeta | 0.8241465 | 0.774097 | 0.5609679 | 0.6169412 | 0.7109001 | 1.1570292 |
| Alpha-prothymosin | 1.1897491 | 1.1462865 | 0.994944 | 1.1447799 | 1.1313958 | 0.7293853 |
| Phase-1 RCT-58 | 0.9074703 | 0.5614373 | 0.8066077 | 0.6756671 | 0.7913678 | 0.7622104 |
| Phase-1 RCT-290 | 1.1756842 | 1.0180892 | 0.9618405 | 0.8015429 | 0.7755446 | 1.0213488 |
| Transitional endoplasmic reticulum ATPase | 1.0982597 | 1.0913838 | 0.9779649 | 1.1317586 | 0.9435568 | 0.9136435 |
| Beta-actin | 0.867344 | 0.6148359 | 0.5694947 | 0.611462 | 0.8109117 | 0.7843312 |
| Thioredoxin-1 (Trx1) | 1.1558306 | 0.9892024 | 1.2522705 | 1.1293892 | 1.0826218 | 1.0490658 |
| Phase-1 RCT-174 | 1.0185992 | 1.0900458 | 1.318991 | 1.065131 | 1.340914 | 0.8673047 |
| Beta-actin, sequence 2 | 1.0779114 | 1.1309184 | 1.0737472 | 1.0886105 | 1.1564051 | 0.8457063 |
| Phase-1 RCT-109 | 1.3319837 | 1.38078 | 1.4821068 | 1.2939512 | 1.3680053 | 0.9210037 |
| Phase-1 RCT-154 | 0.9197751 | 0.9425587 | 0.8319519 | 0.8871394 | 0.7487509 | 1.0680965 |
| Hypoxanthine-guanine | 1.1233082 | 1.0029898 | 0.8737112 | 0.8874736 | 0.944659 | 0.9359556 |
| phosphoribosyltransferase | | | | | | |
| Phase-1 RCT-213 | 0.8591825 | 0.890603 | 1.037875 | 1.1372293 | 1.2065061 | 0.9613363 |
| Phase-1 RCT-260 | 0.8046686 | 0.672716 | 0.8653044 | 0.9582335 | 0.7240526 | 1.0349805 |
| Phase-1 RCT-12 | 0.89719 | 0.8356193 | 0.8374827 | 0.8241765 | 0.8252894 | 0.8679741 |
| Phase-1 RCT-293 | 1.3467244 | 1.1907624 | 1.1550003 | 1.0603774 | 1.1440916 | 0.9000813 |
| Ribosomal protein S8 | 1.5111094 | 1.3435903 | 1.5718602 | 1.445578 | 1.2278337 | 0.8365496 |
| Pyruvate kinase, muscle | 0.842334 | 0.7256505 | 0.9462439 | 0.8174502 | 0.6415882 | 1.0030035 |
| Nucleoside diphosphate kinase beta isoform | 1.3503535 | 1.252334 | 1.3366554 | 1.2706928 | 1.4685682 | 1.004514 |
| p55CDC | 1.0682738 | 1.0253315 | 0.6602161 | 0.7093477 | 0.8900849 | 1.381924 |
| Phase-1 RCT-156 | 1.1761231 | 1.1588861 | 1.1957524 | 0.9654701 | 1.3742363 | 0.8639733 |
| Ribosomal protein L13A | 1.5360535 | 1.4365432 | 1.6355418 | 1.4422266 | 1.6083418 | 0.9997844 |
| Phase-1 RCT-258 | 0.827027 | 0.7948121 | 0.6360102 | 0.9339573 | 0.6929632 | 0.9890729 |
| Insulin-like growth factor I | 1.513679 | 1.3080506 | 1.2366265 | 1.2277861 | 1.5732994 | 0.8894011 |
| Cytochrome P450 3A1 | 1.1705515 | 1.0483115 | 1.5633755 | 1.7201829 | 0.914504 | 0.980382 |
| {Ribosomal protein 16} | 1.2111925 | 1.1840348 | 1.3582498 | 1.030916 | 0.9788765 | 1.0338725 |
| Organic cation transporter 3 | 0.9982783 | 1.3235891 | 0.5662989 | 0.7121626 | 0.8043568 | 0.9417419 |
| Calpain 2 | 0.9090909 | 0.9058917 | 1.0353634 | 0.8022061 | 0.7719548 | 1.0870981 |
| Phase-1 RCT-102 | 1.3336031 | 1.3487008 | 1.1359172 | 1.1768228 | 1.2220567 | 1.0409685 |
| Equilbrative nitrobenzylthioinosine-sensitive | 0.9200042 | 1.0544983 | 0.9362465 | 1.0492365 | 0.8578932 | 1.0098785 |
| nucleoside transporter | | | | | | |
| Phase-1 RCT-227 | 1.0927883 | 1.2711573 | 0.9923551 | 1.1324329 | 1.0679727 | 0.9447098 |
| N-hydroxy-2-acetylaminofluorene sulfotransferase (ST1C1) | 1.0936695 | 1.2702483 | 0.9903241 | 1.0838143 | 1.1098442 | 1.0385723 |
| Cathepsin S | 1.2923077 | 1.1532674 | 1.3343829 | 1.0128534 | 1.4510384 | 1.31053 |
| Apolipoprotein CIII | 1.074966 | 1.4731585 | 1.5217717 | 1.6408852 | 0.9794055 | 1.216628 |
| Cytochrome P450 2A3 | 0.9162771 | 0.760761 | 0.6631087 | 0.8195698 | 0.8166431 | 1.3790195 |
| Cytochrome P450 2C23 | 1.7531288 | 1.4081683 | 1.3464501 | 1.5638318 | 1.1341742 | 0.8166658 |
| Apolipoprotein C1 | 1.2985986 | 1.4390774 | 1.9080673 | 1.6495572 | 1.542801 | 0.8957583 |
| Betaine homocysteine methyltransferase (BHMT) | 1.3127484 | 1.0847023 | 1.1357222 | 0.8464575 | 1.0102075 | 1.0236729 |
| Paraoxonase 1 | 1.4984944 | 1.3507122 | 1.4845159 | 1.4584432 | 1.4298072 | 1.0724077 |
| Phase-1 RCT-207 | 0.835314 | 0.8233585 | 1.0642428 | 1.1331265 | 1.2131498 | 0.9843853 |
| Cathepsin B | 1.6570256 | 1.1180362 | 1.4765229 | 1.1361836 | 1.6397537 | 1.062121 |
| Phase-1 RCT-144 | 0.9797784 | 0.8529155 | 1.0506663 | 0.9174421 | 0.9043912 | 1.0343473 |
| Alpha-1 microglobulin/bikunin precursor (Ambp) | 1.9155146 | 1.7834073 | 1.737669 | 1.7440809 | 1.7327298 | 0.855624 |
| Cyclin D1 | 1.1401956 | 1.5442654 | 1.7053354 | 0.9049702 | 1.503062 | 0.9739355 |
| Presenilin-1 | 1.8967334 | 1.3879656 | 1.2914281 | 0.8188112 | 1.7835559 | 0.953139 |
| Protein tyrosine phosphatase, receptor type, D | 1.5243981 | 1.2487915 | 1.191796 | 1.3673877 | 1.2801254 | 0.8666149 |
| Multidrug resistant protein-1 | 0.8582249 | 0.7058407 | 0.845758 | 0.802066 | 0.764857 | 1.2525847 |
| Cathepsin L, sequence 2 | 1.1689702 | 1.1977792 | 3.8341143 | 1.3349735 | 0.9598955 | 1.0605729 |
| JNK1 stress activated protein kinase | 1.0695219 | 1.0335959 | 0.8937523 | 0.7203483 | 0.6946837 | 1.3208303 |
| Phase-1 RCT-43 | 1.1065162 | 1.140237 | 0.8908395 | 0.8938447 | 0.9235171 | 0.9523817 |
| Tissue plasminogen activator | 0.9078947 | 0.9829918 | 0.9619153 | 0.8867379 | 0.8583804 | 0.9070756 |
| Ubiquitin conjugating enzyme (RAD 6 | 1.0310477 | 1.2950604 | 0.6675518 | 1.298355 | 0.8844897 | 0.9520814 |
| homologue) | | | | | | |
| Phase-1 RCT-173 | 0.7481364 | 0.8414759 | 1.0604639 | 1.3085568 | 1.2420225 | 0.9573818 |
| Ornithine decarboxylase | 0.8604029 | 0.5683225 | 0.5823776 | 0.6899697 | 0.7252544 | 1.1602727 |
| Zinc finger protein | 0.9359543 | 1.1099083 | 0.6014916 | 0.6654311 | 0.7256041 | 0.8131021 |
| CDK108 | 0.8669315 | 0.8640122 | 1.2218041 | 1.1360697 | 1.0576224 | 1.0116482 |
| Phase-1 RCT-117 | 0.7304138 | 1.3967243 | 0.9382309 | 1.2134917 | 1.0609888 | 1.1057533 |
| {Phase-1 RCT 98} | 1.006865 | 1.0757663 | 1.1370387 | 1.1023773 | 1.3310875 | 1.0839231 |
| Aquaporin-3 (AQP3) | 0.8104654 | 0.7708375 | 1.1459538 | 1.0984567 | 1.1716756 | 1.0582706 |
| Cholesterol esterase | 1.5579659 | 1.7463984 | 1.7562032 | 2.075416 | 1.4523927 | 0.8418495 |
| Ribosomal protein S17 | 1.4334096 | 1.2677597 | 1.5359879 | 1.3400729 | 1.3259101 | 0.9071206 |
| Poly(ADP-ribose) polymerase | 0.9497134 | 0.9087828 | 0.8244036 | 0.9169005 | 0.742717 | 1.0390704 |
| Annexin V | 1.036291 | 1.0852834 | 0.7133434 | 0.7938306 | 0.9342983 | 1.3065382 |
| Phase-1 RCT-33 | 1.158662 | 1.0449849 | 1.1741794 | 1.0543674 | 1.238927 | 1.1009438 |
| Phase-1 RCT-61 | 0.8868536 | 0.7071623 | 0.9969032 | 0.9450105 | 0.9195329 | 0.793994 |
| Retinol dehydrogenase type III | 1.3492821 | 1.266464 | 1.2745916 | 1.2525516 | 1.4019663 | 0.8501996 |
| ATPase inhibitor (rat mitochondrial IF1 protein) | 1.3011901 | 1.3365568 | 1.5292716 | 1.4290957 | 1.3852538 | 0.8644434 |
| Thymidylate synthase | 0.8421052 | 0.7777962 | 0.7109714 | 0.5502197 | 0.6380144 | 1.4627135 |
| Interleukin-18 | 0.8911685 | 0.7334618 | 0.5354748 | 0.6707565 | 0.5731996 | 1.2920635 |
| Lecithin: cholesterol acyltransferase | 1.5192174 | 1.4022695 | 1.4197683 | 1.3977686 | 1.3844842 | 0.792896 |
| Contrapsin-like protease inhibitor (CPi-21) | 1.4464076 | 1.3544548 | 1.6105992 | 1.3334309 | 1.3204739 | 0.9160346 |
| Proliferating cell nuclear antigen gene | 1.038547 | 0.7253844 | 0.7989413 | 0.7929193 | 0.8680536 | 1.5091192 |
| Phase-1 RCT-230 | 0.737563 | 0.5372678 | 0.7919134 | 0.8789224 | 0.8176888 | 1.1251643 |
| Cytochrome P450 2D18 | 1.0400596 | 0.9924455 | 0.879772 | 1.0190866 | 1.0404055 | 0.7308574 |
| Phase-1 RCT-48 | 1.3624698 | 1.3464701 | 1.2991847 | 1.2676868 | 1.4239991 | 1.0741946 |
| Phase-1 RCT-292 | 0.8717805 | 0.9213201 | 1.2387094 | 1.0524098 | 1.1727129 | 1.0376415 |
| Arginosuccinate synthetase 1 | 1.1850052 | 0.9140829 | 1.3234562 | 0.8688694 | 1.2321465 | 0.9998384 |
| C-reactive protein | 1.5271555 | 1.2224957 | 1.221843 | 1.3703938 | 1.3762796 | 0.9192518 |

| | ETH | ETH | GAN | GAN | GAN | GAN | GAN |
|---|---|---|---|---|---|---|---|
| Compound-Dose | 2500 | 2500 | 50 | 50 | 50 | 200 | 200 |
| Animal Number | 138 | 139 | 2446 | 2447 | 2448 | 2457 | 2458 |
| Liver Toxicity Necrosis Classification | no | no | no | no | no | no | no |
| Gene Name | | | | | | | |
| Osteoactivin | 0.9867295 | 0.8660413 | 1.0779759 | 0.9888831 | 0.9330887 | 1.0067947 | 1.0937978 |
| Stathmin | 0.9886385 | 1.2155915 | 1.0171176 | 0.9999999 | 0.9212006 | 0.970161 | 0.959928 |
| Calpactin I heavy chain | 0.9979631 | 1.0141376 | 0.9829603 | 1.0602343 | 1.1397586 | 1.120906 | 1.0281618 |
| Phase-1 RCT-179 | 0.9415132 | 0.8705862 | 1.3361789 | 0.9355581 | 0.9850671 | 0.9906796 | 0.9873737 |
| Phase-1 RCT-107 | 0.9165384 | 1.4235811 | 1.0247049 | 1.1036642 | 1.0252148 | 1.1734747 | 1.0761231 |
| Heme oxygenase | 1.1265072 | 1.2650583 | 1.6527284 | 1.1091547 | 0.9900816 | 1.270328 | 1.0740331 |
| Collagen type II | 2.292945 | 1.2513629 | 0.8445768 | 0.9828427 | 0.9876089 | 0.8690317 | 0.8927872 |
| Phase-1 RCT-81 | 1.0320483 | 0.912656 | 0.9798124 | 0.862092 | 0.9675527 | 0.9590582 | 0.9500609 |
| Glycine methyltransferase | 0.8464772 | 1.5014504 | 1.2937168 | 1.4627147 | 1.0691334 | 1.5420008 | 1.6263125 |
| Cystatin C | 0.9618548 | 0.974699 | 0.8636607 | 0.839111 | 1.0804843 | 0.7705706 | 0.8569696 |
| Melanoma-associated antigen ME491 | 1.0684133 | 1.1439472 | 1.2173206 | 0.9545053 | 0.8930039 | 0.9578408 | 0.9743524 |
| Phase-1 RCT-146 | 1.0212888 | 1.4086957 | 1.1149478 | 1.1259813 | 1.0173331 | 1.1029927 | 1.0413145 |
| Gamma-actin, cytoplasmic | 0.8455072 | 0.8528099 | 0.7102713 | 1.2409998 | 1.1031902 | 0.9530774 | 1.05954 |
| Adenine nucleotide translocator 1 | 1.0543376 | 0.8578113 | 0.7796789 | 0.8986606 | 0.9255238 | 0.8342212 | 0.9128272 |
| Thymosin beta-10 | 1.0642501 | 1.1469731 | 0.8932892 | 1.0291269 | 0.8819312 | 0.9392046 | 0.9832799 |
| Insulin-like growth factor I, exon 6 | 0.8065419 | 0.4940214 | 1.3375257 | 0.927959 | 1.1883961 | 1.1190779 | 1.2166548 |
| Phase-1 RCT-78 | 0.9928808 | 0.9787365 | 1.002099 | 0.8931286 | 1.0020434 | 1.0730981 | 1.010598 |
| Alpha-2-microglobulin | 0.5040586 | 0.9079015 | 1.4367491 | 0.577860 | 0.8604845 | 0.8183812 | 0.702008 |
| High affinity IgE receptor gamma chain | 1.11602 | 0.937584 | 0.9808078 | 1.0025076 | 1.0561334 | 0.8745924 | 0.9445157 |
| (FcERIgamma) | | | | | | | |
| 60S ribosomal protein L6 | 0.9039219 | 0.9444764 | 1.3491542 | 1.2082744 | 1.0249484 | 1.0798446 | 1.3120664 |
| IgE binding protein | 1.0244802 | 0.8389378 | 1.0499251 | 1.1443789 | 1.1142975 | 1.2377677 | 1.1097786 |
| Phase-1 RCT-149 | 1.2010533 | 0.9170154 | 0.8978062 | 1.0821066 | 1.0538274 | 1.0679649 | 0.9724227 |
| Cytochrome P450 2C11 | 1.1963612 | 2.0363884 | 0.5508739 | 0.6444258 | 1.0428321 | 1.0736922 | 0.9951202 |
| Uncoupling protein 2 | 1.1095014 | 1.2501696 | 0.7333043 | 0.8795259 | 0.8379349 | 0.6317613 | 0.7680354 |
| Cofilin | 1.060122 | 0.8404279 | 1.0889548 | 1.1118344 | 1.1435035 | 1.01354 | 1.0486804 |
| Alpha-tubulin | 1.1926374 | 0.8850656 | 0.5964575 | 0.987673 | 0.9247625 | 0.7757889 | 0.9065934 |
| Stearyl-CoA desaturase, liver | 1.7262263 | 0.399251 | 0.1395345 | 1.5252275 | 2.5075736 | 1.3618582 | 1.3181487 |
| Glutathione S-transferase theta-1 | 1.018243 | 1.0250423 | 0.7555412 | 0.9249834 | 0.8326972 | 0.8688383 | 0.9016296 |
| alpha-1,2-fucosyltrans-ferase | 1.0235945 | 1.1250589 | 0.8192465 | 1.1970028 | 1.0491648 | 1.0817713 | 1.026357 |
| Phase-1 RCT-138 | 1.0945646 | 0.9314339 | 1.1492808 | 1.0632489 | 1.0169164 | 1.2178202 | 1.1751393 |
| Phase-1 RCT-295 | 1.0915766 | 1.0591547 | 1.1846077 | 1.2289376 | 1.1282351 | 1.3096403 | 1.1887378 |
| Phase-1 RCT-164 | 0.8984169 | 1.6103895 | 0.9491044 | 0.9510998 | 1.086418 | 1.0234885 | 1.0161036 |
| Alpha-2-macroglobulin, sequence 2 | 0.7807344 | 0.9270099 | 1.2930678 | 1.108675 | 1.149934 | 1.4756532 | 1.1781158 |
| Biliverdin reductase | 1.0698495 | 1.8752804 | 0.8870872 | 0.978403 | 0.8443783 | 0.8642948 | 0.8767032 |
| Phase-1 RCT-121 | 0.9692938 | 0.7650203 | 0.9704086 | 0.8816412 | 0.9401888 | 0.8713313 | 0.8968959 |
| Dynamin-1 (D100) | 0.9820597 | 1.028174 | 1.083004 | 1.0541253 | 1.1303124 | 1.0741636 | 1.1259383 |
| Phase-1 RCT-192 | 1.008868 | 0.9559 | 0.9996191 | 1.0000541 | 1.001761 | 0.9338548 | 1.0119838 |
| Fatty acid synthase | 1.8047425 | 0.8426885 | 0.1618168 | 1.2038574 | 1.5134054 | 0.7099712 | 1.0944363 |
| Phase-1 RCT-211 | 0.945088 | 1.1327726 | 0.9828737 | 1.0034038 | 1.0119958 | 1.1910812 | 1.0219636 |
| Ribosomal protein S9 | 1.0877943 | 1.056663 | 0.9500865 | 0.9673258 | 0.8553261 | 0.8455143 | 0.901372 |
| NADP-dependent isocitrate dehydrogenase, | 1.2778752 | 1.0223604 | 1.0136342 | 1.1031204 | 1.0662887 | 1.146447 | 1.1644455 |
| cytosolic | | | | | | | |
| Phase-1 RCT-57 | 0.7637273 | 0.8718218 | 1.0325911 | 0.9680407 | 0.8806684 | 0.9614054 | 0.8913607 |
| Phase-1 RCT-36 | 1.2532121 | 0.9838864 | 0.8994093 | 0.9859248 | 1.0056154 | 1.0089469 | 0.9804115 |
| Apolipoprotein All | 0.7360898 | 1.2430545 | 1.4653546 | 1.3157481 | 1.0944676 | 1.2097143 | 1.075308 |
| Phase-1 RCT-68 | 0.9293349 | 0.8784613 | 0.9114007 | 1.0272478 | 1.0348718 | 0.9426987 | 0.9281483 |
| Multidrug resistant protein-2 | 0.9653397 | 1.72779 | 1.1377316 | 0.9754929 | 0.9633454 | 1.0429456 | 0.9175316 |
| Phase-1 RCT-39 | 0.9208214 | 1.2536978 | 0.914677 | 0.9883627 | 1.0218886 | 1.0389524 | 1.0357932 |
| NIPK | 0.8250896 | 0.7119982 | 1.1231472 | 0.9979691 | 0.9868385 | 1.0108471 | 0.9641562 |
| F1-ATPase beta subunit | 1.0761766 | 0.6967244 | 1.207962 | 1.2725489 | 1.1388601 | 1.0980875 | 0.924623 |
| Elongation factor-1 alpha | 1.0966891 | 0.7776657 | 1.5171865 | 1.090942 | 0.9250763 | 1.1917189 | 1.2584115 |
| Phase-1 RCT-24 | 1.1245649 | 0.9832659 | 0.534285 | 1.2749964 | 1.0972254 | 0.9328807 | 0.9608183 |
| Beta-tubulin, class I | 0.9580033 | 0.8445971 | 0.7030647 | 1.6715432 | 1.4063606 | 1.3251086 | 1.1295447 |
| 14-3-3 zeta | 0.9539694 | 1.3249217 | 0.9619569 | 1.0519402 | 0.9655929 | 0.9173353 | 0.9650767 |
| Alpha-prothymosin | 0.9530411 | 0.83332 | 1.2302432 | 1.0226015 | 1.0947831 | 0.9723582 | 1.0015067 |
| Phase-1 RCT-58 | 0.6004823 | 0.781956 | 0.929265 | 0.6547442 | 0.6139954 | 0.7919478 | 0.7865201 |
| Phase-1 RCT-290 | 0.6882615 | 1.1433301 | 0.5452496 | 1.1805738 | 1.4015647 | 1.4524025 | 1.1000973 |
| Transitional endoplasmic reticulum ATPase | 1.0311681 | 0.9186748 | 0.8180246 | 0.9112889 | 0.9467523 | 0.8625243 | 0.9206248 |
| Beta-actin | 0.8375974 | 0.6349749 | 0.7200688 | 0.9794754 | 0.9624965 | 0.7209308 | 0.9100345 |
| Thioredoxin-1 (Trx1) | 1.0708809 | 1.0597308 | 1.0389798 | 1.0918623 | 1.084297 | 1.2192965 | 1.0980989 |
| Phase-1 RCT-174 | 1.1695801 | 0.6978939 | 0.6971749 | 0.9783595 | 1.1903617 | 1.0944035 | 0.9792113 |
| Beta-actin, sequence 2 | 1.0165979 | 0.8088995 | 0.8398964 | 1.115985 | 1.0728321 | 0.9105901 | 1.1085737 |
| Phase-1 RCT-109 | 1.3111602 | 0.9284993 | 1.23092 | 1.1025732 | 1.0458956 | 1.1411185 | 1.034442 |
| Phase-1 RCT-154 | 1.0321099 | 1.2174611 | 1.0473787 | 1.0327737 | 1.0133255 | 1.0106484 | 1.0258462 |
| Hypoxanthine-guanine | 1.1704164 | 0.6971071 | 0.5713999 | 0.9353993 | 0.9272356 | 0.8806359 | 0.9180563 |
| phosphoribosyltransferase | | | | | | | |
| Phase-1 RCT-213 | 0.9800039 | 1.0625151 | 0.9282232 | 0.945634 | 0.8987551 | 0.8533909 | 0.9127928 |
| Phase-1 RCT-260 | 0.8439538 | 1.0551722 | 1.0049064 | 0.9390845 | 1.000942 | 1.0496457 | 1.0973608 |
| Phase-1 RCT-12 | 0.9714133 | 0.8833922 | 0.8158084 | 1.0937222 | 1.0796448 | 1.0068299 | 0.9871842 |
| Phase-1 RCT-293 | 1.1846547 | 1.0231245 | 0.9972205 | 1.0527238 | 1.0671238 | 0.9935391 | 1.0135531 |
| Ribosomal protein S8 | 0.9083278 | 0.8816187 | 1.4034972 | 1.1939172 | 1.0925965 | 1.4656074 | 1.3369387 |
| Pyruvate kinase, muscle | 0.9795365 | 1.1098646 | 1.0585396 | 0.9924867 | 0.9847296 | 0.998206 | 0.9653978 |
| Nucleoside diphosphate kinase beta isoform | 0.9711704 | 0.9890838 | 0.9304948 | 1.0779191 | 1.0760869 | 1.1468458 | 1.0075041 |
| p55CDC | 1.0279332 | 1.3264277 | 1.1242343 | 0.9409784 | 0.9477983 | 0.9900981 | 1.1303602 |
| Phase-1 RCT-156 | 1.0343488 | 0.7235655 | 0.7737887 | 0.8060936 | 0.9803593 | 0.7366182 | 0.8292096 |
| Ribosomal protein L13A | 0.9502254 | 1.0379235 | 1.0164361 | 1.0721039 | 0.9210273 | 0.9883407 | 1.0165827 |
| Phase-1 RCT-258 | 0.9523528 | 1.1056242 | 1.045369 | 0.9997103 | 0.9328811 | 0.9730906 | 0.9771791 |
| Insulin-like growth factor I | 0.8043357 | 0.6133407 | 1.2847319 | 1.0117508 | 1.0454576 | 1.0696242 | 1.0900186 |
| Cytochrome P450 3A1 | 0.7451075 | 1.149356 | 1.0847957 | 0.5718945 | 0.8926976 | 0.6190397 | 0.8344487 |
| {Ribosomal protein 16} | 1.0898438 | 0.9755232 | 1.3733717 | 1.1861062 | 1.1054885 | 1.3470138 | 1.2537519 |
| Organic cation transporter 3 | 0.9961273 | 0.957561 | 1.0185971 | 1.0618166 | 1.0012413 | 0.937331 | 1.0394182 |
| Calpain 2 | 1.0350738 | 1.1147132 | 1.0519946 | 0.9833736 | 1.0251286 | 1.0840572 | 1.0319333 |
| Phase-1 RCT-102 | 0.9127614 | 1.004695 | 0.6244487 | 0.7665125 | 0.9626114 | 1.0455753 | 0.8344638 |
| Equilbrative nitrobenzylthioinosine-sensitive | 0.7788224 | 0.969321 | 0.9787766 | 0.7902684 | 0.8505445 | 0.9086835 | 0.9712472 |
| nucleoside transporter | | | | | | | |
| Phase-1 RCT-227 | 0.9545195 | 1.0026934 | 1.6356809 | 0.8443374 | 0.9942249 | 1.2770338 | 1.0197562 |
| N-hydroxy-2-acetylaminofluorene sulfotransferase (ST1C1) | 0.7299637 | 0.967945 | 1.4210869 | 0.862047 | 0.8605764 | 1.0188749 | 1.0066175 |
| Cathepsin S | 1.3005396 | 1.358894 | 1.1002234 | 1.1281626 | 0.8521719 | 0.9359313 | 0.9913393 |
| Apolipoprotein CIII | 1.1913962 | 1.0989498 | 1.1601214 | 1.0611722 | 1.0846393 | 0.9953143 | 1.060376 |
| Cytochrome P450 2A3 | 1.0218667 | 1.1847056 | 1.6037476 | 1.6284573 | 1.421989 | 1.5378042 | 1.3848547 |
| Cytochrome P450 2C23 | 0.8823921 | 0.8580837 | 1.6069746 | 1.0240436 | 1.0669886 | 1.2402129 | 1.0951457 |
| Apolipoprotein C1 | 1.0448488 | 0.836301 | 1.4081126 | 0.9109559 | 1.2368876 | 1.1133919 | 1.2644612 |
| Betaine homocysteine methyltransferase (BHMT) | 0.6754579 | 1.1707598 | 0.5806452 | 1.3462048 | 1.6577872 | 1.7302208 | 1.2089865 |
| Paraoxonase 1 | 1.0229981 | 0.9693084 | 1.4703928 | 1.0984068 | 1.0056483 | 1.102828 | 1.1463968 |
| Phase-1 RCT-207 | 0.9799981 | 1.1787572 | 0.9804633 | 0.9475396 | 0.8722201 | 0.9198301 | 0.9132478 |
| Cathepsin B | 1.2132876 | 1.002966 | 1.1077957 | 0.9751138 | 0.9449645 | 0.95901 | 0.9361655 |
| Phase-1 RCT-144 | 1.0335954 | 1.1303204 | 0.8637709 | 0.930825 | 0.9103247 | 0.8912724 | 0.8770263 |
| Alpha-1 microglobulin/bikunin precursor (Ambp) | 1.0749346 | 0.8684676 | 1.4168785 | 0.9743295 | 1.0036223 | 1.2586251 | 1.1350476 |
| Cyclin D1 | 0.8555648 | 1.2889974 | 0.8783828 | 0.9706107 | 0.8934161 | 0.7441357 | 0.8010049 |
| Presenilin-1 | 0.9261433 | 0.7013646 | 2.4946449 | 1.0347936 | 1.0921187 | 1.1786919 | 1.476416 |
| Protein tyrosine phosphatase, receptor type, D | 0.8528667 | 0.7666202 | 1.5296979 | 1.2220734 | 1.351241 | 1.3898754 | 1.2396982 |
| Multidrug resistant protein-1 | 0.9575232 | 1.2458156 | 1.0264012 | 0.9975502 | 0.9491992 | 0.9342286 | 0.844167 |
| Cathepsin L, sequence 2 | 0.8954322 | 1.1255476 | 1.7994686 | 1.2198411 | 0.9833952 | 1.5287038 | 1.2191119 |
| JNK1 stress activated protein kinase | 1.0360928 | 1.5768787 | 1.1641034 | 1.2140715 | 1.0359477 | 1.0565066 | 1.0244865 |
| Phase-1 RCT-43 | 1.0179644 | 0.7893247 | 0.7711342 | 0.8313533 | 0.9280005 | 0.8073501 | 0.8826972 |
| Tissue plasminogen activator | 0.9898228 | 0.8112518 | 1.0563409 | 0.9994006 | 1.0890807 | 1.160264 | 1.0449464 |
| Ubiquitin conjugating enzyme (RAD 6 | 1.0489827 | 1.0326498 | 0.9574584 | 1.1049708 | 0.9590718 | 1.0135493 | 1.1601818 |
| homologue) | | | | | | | |
| Phase-1 RCT-173 | 0.9601581 | 1.1285802 | 0.8838982 | 0.8587131 | 0.8495358 | 0.8132749 | 0.914638 |
| Ornithine decarboxylase | 0.7510849 | 1.0353023 | 1.0875288 | 1.2639751 | 1.0323347 | 1.0894148 | 1.0126089 |
| Zinc finger protein | 1.0621114 | 0.7772508 | 1.0174272 | 0.9557721 | 0.9668986 | 0.928879 | 0.9713312 |
| CDK108 | 0.9379151 | 1.0730135 | 1.050448 | 0.8450586 | 1.1418248 | 0.8936621 | 1.0302818 |
| Phase-1 RCT-117 | 1.0578613 | 1.0704787 | 0.7685138 | 1.2713028 | 1.2364845 | 1.2130892 | 1.2964944 |
| {Phase-1 RCT 98} | 1.0789235 | 0.9978244 | 0.9214799 | 1.0087038 | 1.1265427 | 1.0445516 | 0.9017636 |
| Aquaporin-3 (AQP3) | 0.9640191 | 1.1486506 | 1.0444683 | 0.9919807 | 1.0231365 | 1.1368027 | 0.9938931 |
| Cholesterol esterase | 0.5712329 | 0.7496476 | 0.9033673 | 0.7826392 | 0.7167876 | 0.7103568 | 0.7243522 |
| Ribosomal protein S17 | 1.0553744 | 1 | 1.2943661 | 1.197896 | 1.0435541 | 1.4560019 | 1.2186142 |
| Poly(ADP-ribose) polymerase | 1.0468425 | 1.0480837 | 0.9469108 | 1.0371351 | 0.9419241 | 0.9288195 | 0.8701974 |
| Annexin V | 1.0256557 | 1.2780578 | 1.0252572 | 1.0498611 | 1.0983944 | 1.0088098 | 1.2371365 |
| Phase-1 RCT-33 | 1.2629282 | 0.9347703 | 1.1695337 | 1.0372019 | 1.0662652 | 1.2061621 | 1.2502358 |
| Phase-1 RCT-61 | 0.6830337 | 0.8314152 | 0.8714634 | 0.7508536 | 0.7276457 | 0.784723 | 0.7854787 |
| Retinol dehydrogenase type III | 0.8386483 | 0.8469104 | 1.2202016 | 1.0016595 | 1.2845176 | 1.2116562 | 1.2837799 |
| ATPase inhibitor (rat mitochondrial IF1 protein) | 0.8135107 | 0.8031195 | 1.2223543 | 0.9466059 | 1.1020601 | 1.1604106 | 1.0866631 |
| Thymidylate synthase | 1.0203449 | 1.4427062 | 0.9357413 | 0.8976455 | 0.8756298 | 0.865236 | 1.0292015 |
| Interleukin-18 | 1.0663818 | 1.4086789 | 1.0021391 | 1.0702274 | 0.9685504 | 0.9134071 | 1.0279415 |
| Lecithin: cholesterol acyltransferase | 1.0656267 | 0.8332773 | 1.2776018 | 1.0591547 | 1.0402077 | 0.9513619 | 0.9421642 |
| Contrapsin-like protease inhibitor (CPi-21) | 1.0199839 | 0.8037959 | 2.2178886 | 1.3732824 | 1.0827336 | 1.0502837 | 1.0896784 |
| Proliferating cell nuclear antigen gene | 1.0227515 | 1.7992764 | 0.8703236 | 0.8282647 | 0.763534 | 0.7201996 | 0.7894818 |
| Phase-1 RCT-230 | 0.9578018 | 1.2270108 | 1.1312366 | 1.2040797 | 1.0498688 | 1.2032307 | 1.2263743 |
| Cytochrome P450 2D18 | 0.6709738 | 0.874136 | 1.000943 | 0.8780153 | 0.8756053 | 0.9324529 | 0.8937326 |
| Phase-1 RCT-48 | 0.9781727 | 0.9202653 | 0.9513553 | 0.9663891 | 1.1536391 | 1.1382622 | 1.1081672 |
| Phase-1 RCT-292 | 1.0548565 | 0.9964079 | 1.1374984 | 1.0336142 | 1.0442398 | 1.2079695 | 1.1585839 |
| Arginosuccinate synthetase 1 | 1.2036483 | 1.0964788 | 1.6569113 | 1.2057736 | 1.3022742 | 1.5539439 | 1.4795187 |
| C-reactive protein | 0.8748393 | 0.8203487 | 1.6992137 | 1.248308 | 1.1149913 | 1.3362287 | 1.1921577 |

| | GAN | GEN | GEN | GEN | GEN | GEN |
|---|---|---|---|---|---|---|
| Compound-Dose | 200 | 38 | 38 | 38 | 150 | 150 |
| Animal Number | 2459 | 427 | 428 | 429 | 437 | 438 |
| Liver Toxicity Necrosis Classification | no | no | no | no | no | no |
| Gene Name | | | | | | |
| Osteoactivin | 1.025009 | 0.9802735 | 1.1826694 | 1.156411 | 0.9991484 | 1.0754539 |
| Stathmin | 0.9157791 | 0.9731137 | 0.7863344 | 0.8549556 | 1.0026892 | 0.8101397 |
| Calpactin I heavy chain | 0.855568 | 1.001552 | 0.8553205 | 1.0231395 | 0.8580484 | 0.9963235 |
| Phase-1 RCT-179 | 0.9926973 | 0.9994339 | 1.1257797 | 1.0792457 | 0.9318144 | 1.0579073 |
| Phase-1 RCT-107 | 1.0298804 | 0.9404096 | 0.6739895 | 0.8063681 | 0.9253145 | 1.2351356 |
| Heme oxygenase | 1.219715 | 1.0163785 | 0.8870972 | 0.9625728 | 0.921707 | 0.8659883 |
| Collagen type II | 0.8741542 | 1.1113228 | 0.9754919 | 1.066675 | 1.0323948 | 0.9453684 |
| Phase-1 RCT-81 | 0.9562344 | 1.0399157 | 1.1594027 | 1.0223818 | 1.0537984 | 1.0468619 |
| Glycine methyltransferase | 1.7007111 | 0.6440734 | 0.7316228 | 0.7929047 | 0.9055033 | 1.4954826 |
| Cystatin C | 0.9762675 | 1.0406481 | 1.0578218 | 0.9166271 | 1.1344842 | 1.0939704 |
| Melanoma-associated antigen ME491 | 0.8483532 | 1.0702834 | 1.1267378 | 0.985046 | 0.9418379 | 1.0746545 |
| Phase-1 RCT-146 | 0.9523689 | 0.9744344 | 0.6896707 | 0.7753984 | 1.003929 | 0.8735628 |
| Gamma-actin, cytoplasmic | 0.819104 | 0.9533553 | 1.0170009 | 0.7883285 | 0.7520483 | 0.8894352 |
| Adenine nucleotide translocator 1 | 0.7878216 | 1.0321319 | 1.1358042 | 0.9358121 | 0.9867431 | 0.8865678 |
| Thymosin beta-10 | 0.9668596 | 0.8309688 | 1.2810351 | 1.1150193 | 0.9136022 | 1.2958492 |
| Insulin-like growth factor I, exon 6 | 1.0502431 | 0.9889236 | 1.2628298 | 1.2079836 | 0.8938279 | 0.7629916 |
| Phase-1 RCT-78 | 1.0139669 | 1.1325096 | 1.0011449 | 0.9182543 | 0.9503194 | 1.1087065 |
| Alpha-2-microglobulin | 0.5361544 | 1.1587406 | 1.2157667 | 0.9156412 | 1.4126176 | 1.677096 |
| High affinity IgE receptor gamma chain | 0.9502829 | 0.9258942 | 0.8666652 | 0.9665314 | 1.2060736 | 1.1900371 |
| (FcERIgamma) | | | | | | |
| 60S ribosomal protein L6 | 1.2498583 | 0.9882314 | 1.2680551 | 1.0498663 | 0.7560075 | 0.9518567 |
| IgE binding protein | 1.0258774 | 0.9434594 | 0.8453856 | 0.9094039 | 0.9887276 | 1.6653421 |
| Phase-1 RCT-149 | 1.0110431 | 1.008937 | 0.8876312 | 0.860052 | 1.059866 | 0.9984037 |
| Cytochrome P450 2C11 | 0.6225597 | 1.1582094 | 0.8843622 | 1.030908 | 1.1993448 | 1.103461 |
| Uncoupling protein 2 | 0.8173721 | 0.8992566 | 0.9718789 | 0.9947664 | 0.898794 | 0.9587961 |
| Cofilin | 0.9093896 | 1.028559 | 1.4348117 | 1.1311727 | 1.2308909 | 1.0598533 |
| Alpha-tubulin | 0.7512336 | 0.9576809 | 1.0330559 | 0.8429738 | 1.0080634 | 0.9135685 |
| Stearyl-CoA desaturase, liver | 1.0183637 | 0.9645674 | 1.3311435 | 0.9192911 | 1.3875049 | 0.6971127 |
| Glutathione S-transferase theta-1 | 1.0151753 | 1.0074897 | 1.1619319 | 1.6249726 | 1.1780268 | 1.0130297 |
| alpha-1,2-fucosyltrans-ferase | 1.2031074 | 1.1574273 | 1.7329971 | 1.3197215 | 1.0073321 | 1.0884932 |
| Phase-1 RCT-138 | 1.0602157 | 0.9628277 | 0.8162262 | 0.9033532 | 1.0362072 | 0.9836006 |
| Phase-1 RCT-295 | 1.1926786 | 1.0903615 | 1.0397259 | 1.0232518 | 0.9291124 | 1.0123069 |
| Phase-1 RCT-164 | 0.8595631 | 0.9874787 | 1.2462646 | 1.0845832 | 1.113289 | 1.0548695 |
| Alpha-2-macroglobulin, sequence 2 | 1.3615347 | 0.9367451 | 1.1979707 | 1.0411534 | 1.1966696 | 1.0773816 |
| Biliverdin reductase | 0.8676657 | 0.9033759 | 0.652503 | 0.8415219 | 0.9597346 | 0.9197999 |
| Phase-1 RCT-121 | 0.8743404 | 0.9281453 | 0.7719648 | 0.8344541 | 0.9712067 | 0.9454722 |
| Dynamin-1 (D100) | 1.108143 | 0.9914508 | 1.1510776 | 1.0809184 | 1.1238568 | 1.1226566 |
| Phase-1 RCT-192 | 1.008504 | 0.9610981 | 1.0879668 | 0.9785776 | 0.9311706 | 1.0441685 |
| Fatty acid synthase | 0.8055874 | 1.1358415 | 0.8104605 | 0.6789782 | 0.951699 | 0.6486656 |
| Phase-1 RCT-211 | 1.1284256 | 1.034996 | 0.720474 | 1.0058984 | 0.8275995 | 1.0422662 |
| Ribosomal protein S9 | 0.8713113 | 1.1204343 | 1.2338217 | 1.1087974 | 1.1528183 | 1.0246154 |
| NADP-dependent isocitrate dehydrogenase, | 0.9888127 | 1.1069328 | 1.2641554 | 1.2100265 | 1.3033149 | 1.0329635 |
| cytosolic | | | | | | |
| Phase-1 RCT-57 | 0.8670751 | 0.9155392 | 1.077264 | 0.963445 | 0.8185968 | 1.1366686 |
| Phase-1 RCT-36 | 0.9291032 | 1.0319539 | 1.0872227 | 0.9115518 | 0.8355178 | 1.0250148 |
| Apolipoprotein All | 1.0434442 | 0.9373969 | 1.1620625 | 1.1464187 | 0.8089844 | 1.3080565 |
| Phase-1 RCT-68 | 0.9915881 | 1.0093551 | 1.0923129 | 1.0164686 | 0.9910907 | 1.0859542 |
| Multidrug resistant protein-2 | 0.881282 | 1.1159465 | 0.9026001 | 0.9785776 | 1.2336646 | 1.0063257 |
| Phase-1 RCT-39 | 1.0591553 | 1.0928154 | 0.7602355 | 0.8037033 | 0.8697472 | 0.8912277 |
| NIPK | 0.8658386 | 0.9727841 | 0.9627853 | 0.9882058 | 0.877863 | 0.8214051 |
| F1-ATPase beta subunit | 1.0051346 | 1.0748639 | 1.3651631 | 1.1284653 | 0.9596614 | 1.0766324 |
| Elongation factor-1 alpha | 1.0715662 | 0.9564973 | 1.164818 | 1.2727289 | 0.7340248 | 1.0712153 |
| Phase-1 RCT-24 | 0.824885 | 0.9546064 | 0.915866 | 0.8659056 | 0.9921759 | 0.9605437 |
| Beta-tubulin, class I | 1.0764915 | 0.9106388 | 1.1021557 | 0.8061521 | 1.1081464 | 0.9297447 |
| 14-3-3 zeta | 0.9463571 | 0.9744227 | 0.7835391 | 0.8363957 | 0.9333803 | 0.861092 |
| Alpha-prothymosin | 0.8859322 | 1.0246993 | 1.4722872 | 1.0966278 | 1.0622408 | 1.041074 |
| Phase-1 RCT-58 | 0.7022509 | 0.6535761 | 0.8304284 | 1.342092 | 0.6632724 | 0.7736943 |
| Phase-1 RCT-290 | 1.3968878 | 0.9576809 | 0.7802936 | 1.0199094 | 1.3923291 | 1.0433679 |
| Transitional endoplasmic reticulum ATPase | 0.8346959 | 1.0056673 | 0.8610643 | 0.8681994 | 0.938988 | 0.9145896 |
| Beta-actin | 0.6842319 | 0.6141242 | 0.9148288 | 0.6909313 | 0.6716457 | 0.9646824 |
| Thioredoxin-1 (Trx1) | 1.1031772 | 1.0507355 | 1.1505437 | 0.9887829 | 1.1458477 | 1.1123918 |
| Phase-1 RCT-174 | 0.9916672 | 1.0080937 | 4.0829344 | 1.0513507 | 1.1261249 | 1.1022848 |
| Beta-actin, sequence 2 | 0.8764822 | 0.9590564 | 1.1230154 | 0.8550336 | 0.8294959 | 1.0058373 |
| Phase-1 RCT-109 | 1.0764173 | 1.0665482 | 1.2179714 | 1.1346881 | 0.8316904 | 1.09377 |
| Phase-1 RCT-154 | 1.0248507 | 1.0118642 | 0.770304 | 0.9202583 | 0.9544547 | 0.9885143 |
| Hypoxanthine-guanine | 0.9007714 | 1.070054 | 1.2660183 | 0.9351604 | 0.9277091 | 0.8809908 |
| phosphoribosyltransferase | | | | | | |
| Phase-1 RCT-213 | 0.9410058 | 0.7129813 | 0.8147928 | 0.8756166 | 0.8941792 | 1.1159426 |
| Phase-1 RCT-260 | 1.1895986 | 1.0888671 | 0.7245202 | 0.8100725 | 1.1078931 | 1.0499862 |
| Phase-1 RCT-12 | 0.9041677 | 0.9879339 | 1.0224918 | 0.8728039 | 1.1242185 | 0.949498 |
| Phase-1 RCT-293 | 0.9469374 | 1.0390567 | 1.057252 | 0.9994962 | 0.9254861 | 0.9586751 |
| Ribosomal protein S8 | 1.2824403 | 1.1084317 | 1.3354044 | 1.151473 | 1.1206317 | 1.2643255 |
| Pyruvate kinase, muscle | 1.009956 | 0.8914515 | 1.096003 | 0.9858398 | 0.8603481 | 0.9942079 |
| Nucleoside diphosphate kinase beta isoform | 1.0217357 | 0.9665561 | 1.2511815 | 1.1061574 | 1.0546795 | 1.079027 |
| p55CDC | 0.912043 | 0.9704976 | 1.064472 | 1.1062902 | 1.0280762 | 0.8145025 |
| Phase-1 RCT-156 | 0.797287 | 1.0721052 | 1.2062861 | 1.0197809 | 0.8290314 | 1.0292563 |
| Ribosomal protein L13A | 1.0279016 | 1.0383962 | 1.3101004 | 1.2734498 | 0.744121 | 1.1363084 |
| Phase-1 RCT-258 | 0.9692429 | 0.9947266 | 0.8359475 | 0.8669459 | 0.8770107 | 0.8727984 |
| Insulin-like growth factor I | 0.9621049 | 0.9530124 | 1.2400184 | 1.1244339 | 1.1310556 | 0.9043822 |
| Cytochrome P450 3A1 | 0.524928 | 0.9061869 | 1.1981785 | 1.3803744 | 0.9728684 | 1.9759213 |
| {Ribosomal protein 16} | 1.2266049 | 1.0659791 | 1.2314149 | 1.1171018 | 0.7764008 | 1.0716673 |
| Organic cation transporter 3 | 1.0147967 | 0.9476784 | 1.3037317 | 1.0228802 | 0.7932866 | 0.8442906 |
| Calpain 2 | 1.0121409 | 1.0182467 | 0.9264874 | 0.9069244 | 0.9687088 | 0.9093802 |
| Phase-1 RCT-102 | 0.6809449 | 0.866888 | 0.8817427 | 0.9502778 | 1.1195908 | 1.0187197 |
| Equilbrative nitrobenzylthioinosine-sensitive | 0.8898152 | 1.0600623 | 0.9024738 | 0.9210518 | 1.5609652 | 1.127869 |
| nucleoside transporter | | | | | | |
| Phase-1 RCT-227 | 1.1365902 | 1.1072997 | 1.0235498 | 0.8886303 | 0.9211279 | 1.1616852 |
| N-hydroxy-2-acetylaminofluorene sulfotransferase (ST1C1) | 0.8205065 | 1.111232 | 0.9362689 | 0.9969207 | 1.1862419 | 1.2212319 |
| Cathepsin S | 0.8984011 | 0.916887 | 0.971996 | 0.9595612 | 1.0816596 | 1.0079842 |
| Apolipoprotein CIII | 1.0749013 | 1.0996573 | 1.0785611 | 0.9906359 | 1.1209154 | 1.0829498 |
| Cytochrome P450 2A3 | 1.442365 | 1.1061027 | 0.8703686 | 1.1258904 | 1.308733 | 1.0529666 |
| Cytochrome P450 2C23 | 1.2865385 | 0.8800432 | 1.1983066 | 1.2592862 | 1.298441 | 1.064432 |
| Apolipoprotein C1 | 0.9400049 | 1.0128819 | 1.2538557 | 1.1769068 | 1.8066313 | 1.4283689 |
| Betaine homocysteine methyltransferase (BHMT) | 1.5656614 | 0.9735513 | 0.8118014 | 1.1230943 | 1.5316919 | 1.127557 |
| Paraoxonase 1 | 1.0956572 | 1.0778953 | 1.2665756 | 1.1269002 | 1.6534964 | 1.0833316 |
| Phase-1 RCT-207 | 0.926191 | 0.9137513 | 0.740266 | 0.8002333 | 0.931872 | 1.0971515 |
| Cathepsin B | 1.0158542 | 1.1913154 | 1.134665 | 1.2188491 | 1.0528709 | 1.0558991 |
| Phase-1 RCT-144 | 0.822388 | 0.9311324 | 0.6342596 | 0.7701768 | 1.0730735 | 0.846902 |
| Alpha-1 microglobulin/bikunin precursor (Ambp) | 1.1717973 | 1.1122848 | 1.3361895 | 1.1243858 | 1.0291631 | 1.068309 |
| Cyclin D1 | 0.7330554 | 1.0538383 | 1.1308804 | 0.7108607 | 1.0522084 | 0.8233708 |
| Presenilin-1 | 1.2477536 | 0.7958772 | 1.3624917 | 1.1972228 | 1.5190413 | 0.632739 |
| Protein tyrosine phosphatase, receptor type, D | 1.2858207 | 0.8321691 | 1.2941191 | 1.1313095 | 1.0255325 | 0.9460704 |
| Multidrug resistant protein-1 | 0.845842 | 1.0926293 | 1.0733273 | 1.0481701 | 0.7844509 | 1.0686489 |
| Cathepsin L, sequence 2 | 1.3154045 | 0.9835928 | 1.3738085 | 1.2360723 | 0.7192094 | 1.1580292 |
| JNK1 stress activated protein kinase | 1.0505708 | 0.9557635 | 0.879944 | 0.959617 | 1.3378012 | 1.0058343 |
| Phase-1 RCT-43 | 0.7938551 | 0.9815667 | 1.0393391 | 1.0657113 | 0.8952612 | 0.9635497 |
| Tissue plasminogen activator | 0.991197 | 0.9720997 | 0.9204846 | 0.9429498 | 0.8974763 | 0.9061817 |
| Ubiquitin conjugating enzyme (RAD 6 | 1.077435 | 1.0727031 | 1.2081362 | 1.135216 | 0.9359508 | 1.0837789 |
| homologue) | | | | | | |
| Phase-1 RCT-173 | 0.8473567 | 0.9479392 | 0.8149492 | 0.8953876 | 1.0026357 | 1.1799146 |
| Ornithine decarboxylase | 1.0163472 | 1.0208577 | 0.8746011 | 0.9520629 | 0.6632879 | 0.7784073 |
| Zinc finger protein | 0.9727041 | 0.9721121 | 1.0760905 | 1.2192265 | 1.0117842 | 0.9270515 |
| CDK108 | 0.9149389 | 0.9712633 | 0.7289048 | 0.9248371 | 0.8938471 | 1.0477105 |
| Phase-1 RCT-117 | 1.1926191 | 0.8085395 | 0.6182218 | 1.0045476 | 1.1465044 | 1.0854017 |
| {Phase-1 RCT 98} | 0.9315904 | 1.0122921 | 1.033152 | 1.0543478 | 0.9862849 | 1.0005755 |
| Aquaporin-3 (AQP3) | 0.9811956 | 0.9695901 | 0.836264 | 0.7889617 | 1.0032977 | 1.0756328 |
| Cholesterol esterase | 0.7099956 | 0.8302856 | 1.263695 | 1.1915089 | 0.9991221 | 1.5601908 |
| Ribosomal protein S17 | 1.2211833 | 1.0568415 | 1.4102987 | 1.1405907 | 1.2287242 | 1.2323712 |
| Poly(ADP-ribose) polymerase | 0.9673397 | 1.0195795 | 0.9571172 | 0.9463064 | 1.0171349 | 0.9688042 |
| Annexin V | 1.0555313 | 1.0171762 | 1.1202319 | 0.9440342 | 1.0779487 | 0.9070295 |
| Phase-1 RCT-33 | 1.0314053 | 1.5454814 | 1.1695069 | 0.9342178 | 0.7572409 | 1.1449175 |
| Phase-1 RCT-61 | 0.7956882 | 0.9535277 | 0.7632893 | 1.110932 | 0.8688541 | 0.8724763 |
| Retinol dehydrogenase type III | 1.1433929 | 1.0927871 | 1.4302661 | 1.1108997 | 1.3824258 | 1.1378103 |
| ATPase inhibitor (rat mitochondrial IF1 protein) | 0.862553 | 0.9990029 | 1.4762821 | 1.2506695 | 2.2521565 | 1.3711991 |
| Thymidylate synthase | 0.929954 | 0.9011162 | 0.8049864 | 0.9710732 | 1.0627306 | 0.874877 |
| Interleukin-18 | 1.0931541 | 0.9679689 | 0.7344749 | 0.8963442 | 0.9594095 | 0.7570748 |
| Lecithin: cholesterol acyltransferase | 0.8398013 | 1.1725365 | 1.3841053 | 1.2129834 | 0.7946956 | 1.1578263 |
| Contrapsin-like protease inhibitor (CPi-21) | 1.1902823 | 1.1238031 | 1.4602567 | 0.8386882 | 0.8563817 | 1.1587502 |
| Proliferating cell nuclear antigen gene | 0.7692519 | 1.1033107 | 0.8094212 | 0.9073158 | 1.0954688 | 0.9429589 |
| Phase-1 RCT-230 | 1.2947855 | 1.0717218 | 0.7552124 | 0.8327724 | 1.0256019 | 0.9840903 |
| Cytochrome P450 2D18 | 0.8264161 | 0.899554 | 1.3008807 | 1.051871 | 0.7487723 | 1.1203245 |
| Phase-1 RCT-48 | 1.1627553 | 0.932285 | 1.2133005 | 1.0503718 | 1.3568977 | 1.0888528 |
| Phase-1 RCT-292 | 1.1014278 | 0.9429355 | 0.8639979 | 0.8631222 | 0.9229884 | 0.9785358 |
| Arginosuccinate synthetase 1 | 1.443463 | 1.0584373 | 0.9478664 | 1.1428969 | 0.7202371 | 0.8180893 |
| C-reactive protein | 1.2123493 | 0.9115635 | 1.4404095 | 1.2561878 | 1.0756483 | 1.0535443 |

| | GEN | HYD | HYD | HYD | HYD | HYD | HYD |
|---|---|---|---|---|---|---|---|
| Compound-Dose | 150 | 1000 | 250 | 250 | 250 | 1000 | 1000 |
| Animal Number | 439 | 1237 | 1227 | 1228 | 1229 | 1238 | 1239 |
| Liver Toxicity Necrosis Classification | no | no | no | no | no | no | no |
| Gene Name | | | | | | | |
| Osteoactivin | 1.0705161 | 1.300669 | 1.1093574 | 1.2242782 | 1.0988606 | 1.074457 | 1.0242808 |
| Stathmin | 0.7342157 | 1.0309323 | 1.1323954 | 1.0455275 | 1.1606138 | 1.0988553 | 1.0488884 |
| Calpactin I heavy chain | 0.9582918 | 1.112144 | 1.0864811 | 1.0554715 | 1.0851771 | 1.189245 | 1.2191828 |
| Phase-1 RCT-179 | 0.9146382 | 0.9676533 | 0.8949899 | 0.9822825 | 0.9979798 | 0.9366186 | 0.845666 |
| Phase-1 RCT-107 | 0.7914109 | 0.9483022 | 0.9288526 | 0.9238539 | 0.9724597 | 0.8339028 | 1.0299149 |
| Heme oxygenase | 0.9439424 | 1.2396042 | 1.322709 | 1.3939224 | 1.1699336 | 1.2367092 | 1.48297 |
| Collagen type II | 0.7355987 | 0.8935058 | 0.8971446 | 0.8841631 | 0.9022246 | 1.0834676 | 1.0065354 |
| Phase-1 RCT-81 | 1.0076666 | 0.9798861 | 0.9433954 | 1 | 0.9619154 | 0.9796673 | 1.0065745 |
| Glycine methyltransferase | 0.8396564 | 0.8764489 | 0.7574071 | 0.6475878 | 0.9352296 | 0.4653186 | 0.8855225 |
| Cystatin C | 0.8694426 | 0.9023272 | 0.9862825 | 1.1459326 | 1.0246291 | 1.1243123 | 0.9065915 |
| Melanoma-associated antigen ME491 | 0.985447 | 1.6418222 | 1.108302 | 1.0918578 | 1.1615111 | 1.3533549 | 1.1491164 |
| Phase-1 RCT-146 | 0.7550023 | 1.2429338 | 1.7968428 | 1.4474351 | 1.4390094 | 1.7573133 | 1.358435 |
| Gamma-actin, cytoplasmic | 0.9408523 | 1.0951501 | 1.0090821 | 0.8560529 | 1.039812 | 0.9724654 | 1.0166988 |
| Adenine nucleotide translocator 1 | 0.6792793 | 0.8600286 | 0.7245378 | 0.7069881 | 0.9453445 | 0.9343067 | 0.8345177 |
| Thymosin beta-10 | 0.9837859 | 0.7099458 | 0.7594228 | 0.7723865 | 0.789062 | 0.7420216 | 0.6943318 |
| Insulin-like growth factor I, exon 6 | 1.3272629 | 1 | 0.7673897 | 0.7564625 | 0.8652008 | 0.6434059 | 1.0443228 |
| Phase-1 RCT-78 | 1.0066952 | 1.0979191 | 1.0345168 | 1.1093253 | 1.0050827 | 1.1207622 | 1.0909106 |
| Alpha-2-microglobulin | 1.1380059 | 0.6494808 | 0.5051327 | 0.6881037 | 0.6943252 | 0.3610158 | 0.3978469 |
| High affinity IgE receptor gamma chain | 1.0210228 | 1.241382 | 1.0401264 | 1.1576936 | 1.1004995 | 1.1622772 | 0.9225797 |
| (FcERIgamma) | | | | | | | |
| 60S ribosomal protein L6 | 1.0160515 | 0.9296418 | 0.8667292 | 0.8988959 | 0.9935561 | 0.5710954 | 0.5978537 |
| IgE binding protein | 0.8563736 | 1.0056298 | 1.1557009 | 1.1946119 | 1.0742306 | 1.0637853 | 1.1314516 |
| Phase-1 RCT-149 | 1.0951236 | 0.9958141 | 0.9825343 | 1.0747501 | 1.0480634 | 1.1077769 | 1.0507643 |
| Cytochrome P450 2C11 | 0.9878716 | 0.9149127 | 1.7610223 | 1.1643682 | 1.3231387 | 1.5715137 | 1.4269174 |
| Uncoupling protein 2 | 0.7531481 | 1.0439873 | 1.0613145 | 1.1099236 | 0.9906051 | 1.1245694 | 1.0282019 |
| Cofilin | 0.9575366 | 1.3573351 | 0.9456023 | 1.0956489 | 1.1438875 | 1.1218064 | 1.0201671 |
| Alpha-tubulin | 0.6678408 | 0.7619216 | 0.6915101 | 0.7903025 | 0.9947382 | 1.0067128 | 0.851471 |
| Stearyl-CoA desaturase, liver | 0.9577755 | 0.3847889 | 0.3819303 | 1.3667542 | 1.0490593 | 0.4773804 | 0.5074706 |
| Glutathione S-transferase theta-1 | 1.1383275 | 0.807542 | 0.8154615 | 0.878427 | 0.9647585 | 0.8230969 | 0.6925434 |
| alpha-1,2-fucosyltrans-ferase | 1.0543368 | 0.5895538 | 0.9435945 | 0.8774352 | 0.5794762 | 0.6103986 | 0.629275 |
| Phase-1 RCT-138 | 1.0115153 | 1.077148 | 0.9472486 | 1.056592 | 1.0885966 | 0.9309651 | 0.9155292 |
| Phase-1 RCT-295 | 1.0426207 | 1.0944687 | 0.9821643 | 1.0423484 | 0.9729955 | 0.8437638 | 0.9582278 |
| Phase-1 RCT-164 | 0.8434994 | 0.9253251 | 1.0156665 | 0.8532649 | 1 | 1.000542 | 1.020412 |
| Alpha-2-macroglobulin, sequence 2 | 1.0200013 | 1.1886684 | 1.2751654 | 1.3156694 | 1.0122751 | 1.0121809 | 1.1464114 |
| Biliverdin reductase | 0.9163266 | 1.1232477 | 1.3846731 | 1.0694534 | 1.112238 | 1.3168821 | 1.1545552 |
| Phase-1 RCT-121 | 0.6518759 | 1.0624646 | 1.2389818 | 1.1590191 | 1.2677963 | 1.1716961 | 1.1840235 |
| Dynamin-1 (D100) | 1.0708693 | 0.9648704 | 1.0206808 | 1.163745 | 1.0504559 | 0.953796 | 0.9046612 |
| Phase-1 RCT-192 | 1.0842748 | 1.1126225 | 0.8604381 | 1.0432427 | 0.9470066 | 0.8082399 | 0.7577254 |
| Fatty acid synthase | 0.7286815 | 0.7196854 | 0.8887082 | 1.207868 | 1.0613443 | 1.221173 | 0.9523765 |
| Phase-1 RCT-211 | 1.0191344 | 0.6959946 | 0.8846728 | 0.843048 | 0.7648602 | 0.6156802 | 0.7674581 |
| Ribosomal protein S9 | 1.05061 | 1.0007952 | 0.7596133 | 0.9280862 | 1.0046672 | 0.9287631 | 0.8370051 |
| NADP-dependent isocitrate dehydrogenase, | 1.1423898 | 0.9780203 | 0.8949908 | 1.0924201 | 1.0057421 | 0.932247 | 0.9817763 |
| cytosolic | | | | | | | |
| Phase-1 RCT-57 | 0.9155448 | 0.9207563 | 1.039169 | 0.8592768 | 0.930431 | 1.575717 | 1.2096218 |
| Phase-1 RCT-36 | 1.046465 | 0.886386 | 0.9193174 | 1.0282153 | 0.9640654 | 0.9370874 | 0.9895666 |
| Apolipoprotein All | 1.0734365 | 0.3170188 | 0.5807542 | 0.6931565 | 0.5094268 | 0.2505194 | 0.4602235 |
| Phase-1 RCT-68 | 1.0318122 | 1.1973742 | 1.0930483 | 1.1457831 | 1.1326951 | 1.1958752 | 1.1232314 |
| Multidrug resistant protein-2 | 0.9693445 | 1.0331792 | 1.0446156 | 0.8116767 | 0.7209227 | 0.8766241 | 1.0517021 |
| Phase-1 RCT-39 | 0.8344957 | 1.0144529 | 0.9531245 | 1.0829902 | 0.9539586 | 0.9781212 | 0.897521 |
| NIPK | 1.0315771 | 1.0780861 | 0.9756346 | 0.9899865 | 1.0128294 | 1.0751258 | 1.0942725 |
| F1-ATPase beta subunit | 1.1208291 | 0.8809245 | 0.8843047 | 0.8617542 | 0.8648095 | 0.7337955 | 0.8148158 |
| Elongation factor-1 alpha | 1.2102404 | 0.8442539 | 0.8616974 | 0.8179432 | 0.843641 | 0.8618712 | 0.8684429 |
| Phase-1 RCT-24 | 0.9708305 | 1.1866398 | 0.9318054 | 1.0492853 | 0.9740011 | 0.866812 | 0.8879958 |
| Beta-tubulin, class I | 0.8900703 | 1.264087 | 0.968064 | 1.228812 | 0.9561493 | 0.8223152 | 0.8174597 |
| 14-3-3 zeta | 0.8129708 | 1.1434753 | 0.89793 | 0.9347163 | 1.0358732 | 1.4408547 | 0.8804671 |
| Alpha-prothymosin | 0.9847774 | 1.0450599 | 0.8600683 | 0.9994018 | 1.0737407 | 0.8127706 | 0.7783665 |
| Phase-1 RCT-58 | 1.0272071 | 0.497419 | 0.4904625 | 0.6650732 | 0.4649202 | 0.3996176 | 0.4314632 |
| Phase-1 RCT-290 | 0.9881342 | 0.8091722 | 0.9156399 | 1.1610914 | 0.9130394 | 0.683329 | 0.9352207 |
| Transitional endoplasmic reticulum ATPase | 0.7842943 | 1.236915 | 0.9908237 | 1.1144913 | 1.1912084 | 1.156463 | 1.0626335 |
| Beta-actin | 0.9048969 | 1.0145978 | 0.6965613 | 0.7530984 | 1.0266814 | 0.7023559 | 0.8076389 |
| Thioredoxin-1 (Trx1) | 0.9616216 | 0.9011136 | 1.0545635 | 1.087984 | 0.8606996 | 0.7110368 | 0.7052043 |
| Phase-1 RCT-174 | 1.0045234 | 1.0318487 | 0.9567832 | 1.0553787 | 1.0509667 | 1.0369632 | 0.9488115 |
| Beta-actin, sequence 2 | 1.0573671 | 1.1173711 | 0.8777658 | 0.9737741 | 1.0412716 | 0.858004 | 0.9434004 |
| Phase-1 RCT-109 | 1.0472778 | 0.7350944 | 0.8414477 | 0.9411005 | 0.7544789 | 0.6282281 | 0.6484602 |
| Phase-1 RCT-154 | 0.6978671 | 1.0714726 | 1.1690426 | 1.1594939 | 1.0921053 | 1.53607 | 1.0777861 |
| Hypoxanthine-guanine | 0.8615011 | 0.9291766 | 0.7986246 | 0.9335969 | 0.8972337 | 0.8776358 | 0.8295425 |
| phosphoribosyltransferase | | | | | | | |
| Phase-1 RCT-213 | 0.8762786 | 1.0852933 | 1.1096374 | 0.9720342 | 1.2430694 | 1.1789044 | 1.0412879 |
| Phase-1 RCT-260 | 0.939796 | 0.9330619 | 1.0695688 | 1.0365821 | 0.9856951 | 1.2020714 | 1.0333705 |
| Phase-1 RCT-12 | 0.9043849 | 1.0740346 | 0.9139475 | 1.1269006 | 1.0064938 | 0.9739018 | 0.9367859 |
| Phase-1 RCT-293 | 1.0032208 | 1.0191917 | 1.0584232 | 1.1589872 | 1.1019789 | 1.0956976 | 0.9303865 |
| Ribosomal protein S8 | 1.0396814 | 1.054328 | 1.0024048 | 1.0336319 | 0.776548 | 0.8203899 | 0.7555876 |
| Pyruvate kinase, muscle | 0.9557056 | 0.9364871 | 0.9380005 | 1.0635214 | 0.8996353 | 0.8894374 | 0.8797815 |
| Nucleoside diphosphate kinase beta isoform | 1.0521227 | 1.0763149 | 0.8027196 | 0.9548351 | 0.9144136 | 0.8863588 | 0.8564013 |
| p55CDC | 0.9878539 | 0.9070496 | 0.9113011 | 0.839082 | 0.8565532 | 1.2296114 | 1.1346927 |
| Phase-1 RCT-156 | 1.0655539 | 0.8991171 | 0.7652895 | 0.9743834 | 0.9709083 | 0.8372231 | 0.9805147 |
| Ribosomal protein L13A | 1.208862 | 0.8014152 | 0.9062606 | 0.9153389 | 0.7134299 | 0.6314735 | 0.6709577 |
| Phase-1 RCT-258 | 0.6802109 | 0.9962202 | 1.0336224 | 1.0199239 | 1.1290689 | 1.080005 | 0.9728178 |
| Insulin-like growth factor I | 1.3602372 | 0.9555663 | 0.8262244 | 0.765341 | 0.7733741 | 0.6923794 | 1.0094556 |
| Cytochrome P450 3A1 | 1.3683447 | 0.996496 | 0.700127 | 0.7000693 | 0.864153 | 0.6072736 | 0.5521326 |
| {Ribosomal protein 16} | 1.138266 | 0.9107056 | 0.9052654 | 0.9951622 | 0.7881563 | 0.805965 | 0.8612535 |
| Organic cation transporter 3 | 0.7115037 | 0.9653843 | 0.8399712 | 0.92343 | 0.9669904 | 0.8600988 | 0.8526347 |
| Calpain 2 | 1.0221417 | 1.0837089 | 1.1158221 | 1.1024749 | 0.9968404 | 1.0478852 | 1.0083041 |
| Phase-1 RCT-102 | 0.9555004 | 1.1837685 | 0.8806973 | 0.8321493 | 1.0281234 | 0.9133582 | 1.1610223 |
| Equilbrative nitrobenzylthioinosine-sensitive | 1.0570862 | 1.0043843 | 0.9200387 | 0.9421167 | 0.913637 | 0.8124979 | 0.7243218 |
| nucleoside transporter | | | | | | | |
| Phase-1 RCT-227 | 1.0080024 | 1.0966179 | 1.054566 | 1.1679405 | 1.0021831 | 0.8244389 | 0.8428804 |
| N-hydroxy-2-acetylaminofluorene sulfotransferase (ST1C1) | 1.1463763 | 1.0180728 | 0.9005262 | 0.9083827 | 0.880648 | 0.7773409 | 0.6957301 |
| Cathepsin S | 1.0423189 | 1.0871462 | 0.8966126 | 0.8774834 | 0.746973 | 1.0572885 | 0.8934793 |
| Apolipoprotein CIII | 0.8656471 | 0.6824046 | 0.8023034 | 0.9327446 | 0.9351466 | 1.0513904 | 0.8086248 |
| Cytochrome P450 2A3 | 0.973798 | 0.6384148 | 1.4729716 | 0.9372354 | 1.3322848 | 0.8461095 | 0.872293 |
| Cytochrome P450 2C23 | 1.1463947 | 0.9439095 | 1.0327848 | 0.9814393 | 0.9565458 | 0.8484219 | 0.9060404 |
| Apolipoprotein C1 | 1.018908 | 0.8650643 | 0.8833664 | 0.9825961 | 1.159985 | 0.9044399 | 0.7444707 |
| Betaine homocysteine methyltransferase (BHMT) | 1.062888 | 0.4967659 | 0.6175002 | 0.9272843 | 0.7573558 | 0.4306027 | 0.7858356 |
| Paraoxonase 1 | 1.078891 | 1.0519454 | 1.0505798 | 1.0020653 | 0.8660561 | 0.9207841 | 0.7604672 |
| Phase-1 RCT-207 | 0.8690403 | 0.9151627 | 1.0355797 | 0.9910525 | 1.0285404 | 0.9848231 | 0.8956582 |
| Cathepsin B | 1.1175225 | 0.9110707 | 0.9515519 | 0.8863336 | 0.8572992 | 0.8043236 | 0.8635764 |
| Phase-1 RCT-144 | 0.8257756 | 1.1728516 | 1.0865451 | 1.2078208 | 1.1315463 | 1.3384553 | 0.9328331 |
| Alpha-1 microglobulin/bikunin precursor (Ambp) | 1.0268582 | 0.9666763 | 0.9285816 | 1.0114069 | 0.9575377 | 1.0158962 | 0.9766012 |
| Cyclin D1 | 1.1047227 | 1.2019799 | 1.0112745 | 0.8371793 | 1.3274584 | 1.8547151 | 1.420534 |
| Presenilin-1 | 1.5125183 | 1.015638 | 0.8797813 | 0.9871894 | 0.9209028 | 0.9184654 | 0.7253684 |
| Protein tyrosine phosphatase, receptor type, D | 1.0744398 | 1.1953216 | 1.2395912 | 1.038154 | 1.035803 | 0.9628901 | 1.1964022 |
| Multidrug resistant protein-1 | 1.0062871 | 0.9899074 | 1.0520599 | 0.7876397 | 0.8503421 | 0.9569328 | 1.0288575 |
| Cathepsin L, sequence 2 | 1.0813105 | 0.8960887 | 0.9543442 | 0.8380362 | 0.7317135 | 0.7046767 | 0.891625 |
| JNK1 stress activated protein kinase | 0.9230216 | 0.7070149 | 0.9665416 | 0.818183 | 0.7825006 | 0.8703184 | 0.9933229 |
| Phase-1 RCT-43 | 0.8576198 | 0.8352377 | 0.8190164 | 0.9048715 | 1.0101801 | 0.8081096 | 0.9284086 |
| Tissue plasminogen activator | 0.9612001 | 1.0360544 | 1.1548795 | 1.1202401 | 1.0666279 | 1.330973 | 1.1806129 |
| Ubiquitin conjugating enzyme (RAD 6 | 1.0096983 | 0.8966786 | 0.7407783 | 0.8526225 | 1.036951 | 0.8965784 | 0.8620674 |
| homologue) | | | | | | | |
| Phase-1 RCT-173 | 0.904216 | 0.7594053 | 1.0390757 | 0.9259898 | 0.9700977 | 1.0101525 | 0.7608692 |
| Ornithine decarboxylase | 0.9702193 | 1.2240186 | 1.023057 | 0.9595559 | 1.2010803 | 1.1668957 | 1.131591 |
| Zinc finger protein | 0.7188853 | 0.8969652 | 1.1714935 | 1.0335448 | 1.1620849 | 0.9922017 | 0.9519292 |
| CDK108 | 0.9837002 | 0.7666991 | 0.9461453 | 0.9475619 | 0.8451484 | 0.7459991 | 1.3644282 |
| Phase-1 RCT-117 | 0.887525 | 0.9577426 | 0.8768188 | 1.1142853 | 0.8802534 | 0.8187872 | 0.9726572 |
| {Phase-1 RCT 98} | 1.057966 | 1.015492 | 0.9973239 | 1.167825 | 1.1131845 | 1.1816576 | 1.1931558 |
| Aquaporin-3 (AQP3) | 1.022894 | 1.1409945 | 1.1309035 | 1.300238 | 1.1355637 | 1.234099 | 1.0903064 |
| Cholesterol esterase | 1.2475002 | 0.848534 | 0.701579 | 0.5969587 | 0.6211327 | 0.5944177 | 0.5208119 |
| Ribosomal protein S17 | 1.2027469 | 1.1960557 | 1.0965687 | 1.1602547 | 0.9483621 | 0.9077254 | 0.8662642 |
| Poly(ADP-ribose) polymerase | 0.8038477 | 1.0409384 | 0.9682379 | 1.0731474 | 1.0832943 | 1.2128718 | 1.0137104 |
| Annexin V | 0.9801698 | 0.9303072 | 1.0046785 | 0.8106929 | 0.9115061 | 1.1633989 | 1.0547216 |
| Phase-1 RCT-33 | 1.2658854 | 0.7983656 | 0.8248606 | 0.8898896 | 0.8739635 | 0.7029713 | 0.8779658 |
| Phase-1 RCT-61 | 0.9087172 | 0.8112056 | 0.9159605 | 0.9408976 | 1.2719415 | 0.9458501 | 0.9218224 |
| Retinol dehydrogenase type III | 1.1073469 | 0.8895601 | 0.8237198 | 0.9018384 | 0.9007297 | 0.7441775 | 0.9208385 |
| ATPase inhibitor (rat mitochondrial IF1 protein) | 1.2704374 | 1.0680581 | 0.8029156 | 0.9523318 | 0.8698847 | 0.7562925 | 0.7984131 |
| Thymidylate synthase | 0.7541774 | 1.1158949 | 1.1392577 | 0.9414575 | 1.0206054 | 1.5166309 | 1.3221132 |
| Interleukin-18 | 0.5600268 | 1.1765939 | 1.3787025 | 1.1638584 | 1.3401647 | 1.550532 | 1.3725853 |
| Lecithin: cholesterol acyltransferase | 1.1617441 | 0.8564058 | 0.7790029 | 1.012961 | 0.8439493 | 0.7443656 | 0.8112187 |
| Contrapsin-like protease inhibitor (CPi-21) | 1.1353345 | 1.2914234 | 0.9508278 | 0.9879451 | 0.8671765 | 0.9347477 | 1.2791157 |
| Proliferating cell nuclear antigen gene | 1.0088004 | 0.9653844 | 1.2628955 | 1.0001093 | 0.9472914 | 1.5824833 | 1.1940829 |
| Phase-1 RCT-230 | 0.8760529 | 0.9780446 | 1.2130462 | 1.1968086 | 0.9884104 | 1.0774236 | 1.0567791 |
| Cytochrome P450 2D18 | 1.0004123 | 1.0882052 | 0.7864631 | 0.7790595 | 0.7965286 | 0.6764764 | 0.8482089 |
| Phase-1 RCT-48 | 1.1428002 | 0.9378542 | 1.0588995 | 1.107826 | 1.2504753 | 1.0207504 | 0.9519075 |
| Phase-1 RCT-292 | 1.0196528 | 1.0107579 | 0.9878227 | 1.1023988 | 1.1535336 | 1.178217 | 0.9330547 |
| Arginosuccinate synthetase 1 | 1.053717 | 0.5870425 | 0.8149675 | 0.9724749 | 0.9363518 | 0.6384552 | 0.8824444 |
| C-reactive protein | 1.1579852 | 1.0432535 | 1.0087737 | 0.8036276 | 0.8104883 | 0.9924637 | 1.0412433 |

| | ISON | ISON | ISON | ISON | ISON | ISON |
|---|---|---|---|---|---|---|
| Compound-Dose | 50 | 50 | 50 | 200 | 200 | 200 |
| Animal Number | 1947 | 1948 | 1949 | 1957 | 1958 | 1959 |
| Liver Toxicity Necrosis Classification | no | no | no | no | no | no |
| Gene Name | | | | | | |
| Osteoactivin | 1.0941331 | 0.9679047 | 1.0258203 | 1.0556896 | 1.2330841 | 1.157032 |
| Stathmin | 1.0471414 | 0.9208693 | 0.9764895 | 0.954189 | 0.9492639 | 1.0049642 |
| Calpactin I heavy chain | 0.8461877 | 0.8057514 | 0.9669713 | 0.9309067 | 0.8833696 | 0.8508245 |
| Phase-1 RCT-179 | 0.9627715 | 0.8373591 | 0.9494024 | 0.9629125 | 0.859571 | 0.931163 |
| Phase-1 RCT-107 | 1.0104604 | 1.0258583 | 1.1820911 | 1.1931278 | 1.0206343 | 1.0351357 |
| Heme oxygenase | 1.0470786 | 0.9709157 | 1.0647852 | 0.7914852 | 0.9820598 | 0.8776706 |
| Collagen type II | 0.6514542 | 0.7143802 | 0.8940364 | 0.6746154 | 0.6951712 | 0.5613089 |
| Phase-1 RCT-81 | 1.510758 | 1.0398685 | 0.9800444 | 0.9978403 | 1.030735 | 1.0455397 |
| Glycine methyltransferase | 0.9199653 | 0.8889996 | 1.6872816 | 1.8633434 | 1.1439337 | 0.8867357 |
| Cystatin C | 0.8704144 | 0.7026129 | 0.904734 | 0.6879881 | 0.8251435 | 0.8517996 |
| Melanoma-associated antigen ME491 | 0.9480044 | 0.8570867 | 1.1711934 | 0.8710998 | 1.0608941 | 0.8789127 |
| Phase-1 RCT-146 | 1.0046166 | 1.0213746 | 1.0009739 | 0.9192686 | 0.9919388 | 1.0510613 |
| Gamma-actin, cytoplasmic | 1.1537331 | 1.0010753 | 0.7059661 | 0.9812565 | 0.7499938 | 0.8185252 |
| Adenine nucleotide translocator 1 | 0.4728931 | 0.4868754 | 0.4803052 | 0.4796457 | 0.4621695 | 0.4619936 |
| Thymosin beta-10 | 0.8504694 | 0.8471915 | 1.0671715 | 1.0459778 | 0.9303151 | 0.8290003 |
| Insulin-like growth factor I, exon 6 | 1.0073394 | 1.0590205 | 1.0082982 | 1.2535574 | 0.8298702 | 0.9194508 |
| Phase-1 RCT-78 | 1.022914 | 1.0905014 | 0.9615251 | 0.9899789 | 1.0097878 | 0.9099597 |
| Alpha-2-microglobulin | 0.7553653 | 0.3552694 | 1.007772 | 0.752263 | 0.4818663 | 0.7275024 |
| High affinity IgE receptor gamma chain | 0.8743646 | 0.888869 | 0.9021323 | 0.8838586 | 1.0194733 | 1.0120776 |
| (FcERIgamma) | | | | | | |
| 60S ribosomal protein L6 | 0.8244419 | 0.8344173 | 0.8330619 | 0.7715231 | 0.810666 | 0.8173761 |
| IgE binding protein | 0.857666 | 0.8568784 | 0.9134148 | 0.87703 | 0.9575555 | 0.8676343 |
| Phase-1 RCT-149 | 1.0344175 | 1.0145648 | 1.1792209 | 1.112184 | 1.0196267 | 1.0465854 |
| Cytochrome P450 2C11 | 0.7825109 | 0.8615817 | 0.8995948 | 0.7627285 | 0.8309621 | 0.8831849 |
| Uncoupling protein 2 | 1.0368588 | 1.0065967 | 1.1526465 | 1.0611308 | 1.0439643 | 0.9004821 |
| Cofilin | 1.0301483 | 0.9227098 | 0.9108188 | 1.0554643 | 0.9972287 | 0.9519653 |
| Alpha-tubulin | 0.7583956 | 0.8765973 | 0.8201845 | 0.8973793 | 0.6681554 | 0.7486613 |
| Stearyl-CoA desaturase, liver | 0.878129 | 1.4761044 | 0.7941261 | 1.91457 | 1.6175625 | 0.9338408 |
| Glutathione S-transferase theta-1 | 0.925329 | 0.8431193 | 0.7874799 | 0.9914954 | 0.9041769 | 0.8699092 |
| alpha-1,2-fucosyltrans-ferase | 1.4017379 | 1.0546166 | 0.9496441 | 1.208756 | 1.1086963 | 1.1218169 |
| Phase-1 RCT-138 | 0.9251018 | 0.8960264 | 0.9476354 | 1.1109831 | 1.0746642 | 1.0256257 |
| Phase-1 RCT-295 | 1.0424485 | 0.9891527 | 0.9927566 | 1.1089727 | 0.9760979 | 1.1326138 |
| Phase-1 RCT-164 | 1.0390987 | 1.0663508 | 1.0163997 | 1.0821102 | 1.0977409 | 1.047336 |
| Alpha-2-macroglobulin, sequence 2 | 0.9431298 | 0.9449178 | 0.8953831 | 0.9252971 | 0.8815969 | 0.8839033 |
| Biliverdin reductase | 1.055083 | 0.9834974 | 0.9461063 | 1.0203062 | 1.0208349 | 1.0866367 |
| Phase-1 RCT-121 | 0.9899446 | 0.9841719 | 0.9946247 | 0.9484252 | 1.0323154 | 0.9396453 |
| Dynamin-1 (D100) | 0.9886144 | 0.9982142 | 1.0112592 | 0.9614385 | 1.0309279 | 0.9937413 |
| Phase-1 RCT-192 | 0.9816001 | 0.9577834 | 0.9520295 | 1.014138 | 0.9570782 | 0.9630659 |
| Fatty acid synthase | 1.0873084 | 1.772227 | 0.8383288 | 1.5224847 | 1.1625149 | 0.7754391 |
| Phase-1 RCT-211 | 1.1589959 | 1.0879102 | 1.3417999 | 1.4415586 | 1.2129091 | 1.3378177 |
| Ribosomal protein S9 | 0.9103035 | 0.8104092 | 0.8356946 | 0.8121683 | 0.8050262 | 0.8423166 |
| NADP-dependent isocitrate dehydrogenase, | 0.9637404 | 1.1173693 | 0.8410283 | 1.0912653 | 1.0364336 | 1.0133063 |
| cytosolic | | | | | | |
| Phase-1 RCT-57 | 1.0879842 | 1.0139323 | 1.0450219 | 1.059038 | 1.0275029 | 0.9689808 |
| Phase-1 RCT-36 | 0.9688776 | 0.967136 | 0.9370335 | 1.1376665 | 1.018274 | 1.0370675 |
| Apolipoprotein All | 0.7558178 | 0.8997209 | 1.0624983 | 1.7874542 | 1.3501084 | 1.0162449 |
| Phase-1 RCT-68 | 1.0234134 | 1.0023066 | 1.0443586 | 1.1055864 | 1.0512122 | 1.1094847 |
| Multidrug resistant protein-2 | 0.9513398 | 1.0366532 | 1.0207151 | 0.9878545 | 0.9300833 | 1.1164813 |
| Phase-1 RCT-39 | 1.0055455 | 1.0469059 | 1.070373 | 1.0431736 | 1.054677 | 1.0117857 |
| NIPK | 1.0335556 | 1.0804247 | 1.0910281 | 1.1529243 | 0.9420178 | 1.0485353 |
| F1-ATPase beta subunit | 0.9737771 | 1.0489725 | 1.0602832 | 0.9859331 | 0.9698805 | 0.8968875 |
| Elongation factor-1 alpha | 0.924074 | 0.9499499 | 1.1215204 | 0.9755131 | 0.9609393 | 0.8204778 |
| Phase-1 RCT-24 | 1.0590435 | 1.3424087 | 1.1562974 | 1.417487 | 1.0491544 | 1.085146 |
| Beta-tubulin, class I | 1.1444376 | 1.3345822 | 1.094264 | 1.5873781 | 0.8874254 | 1.0625975 |
| 14-3-3 zeta | 1.0025386 | 1.043877 | 1.107657 | 0.9511764 | 0.9930174 | 1.0079476 |
| Alpha-prothymosin | 1.0921605 | 0.8412436 | 0.8698465 | 0.7400106 | 0.8766198 | 0.7101488 |
| Phase-1 RCT-58 | 1.4532794 | 1.3714343 | 1.4752942 | 1.6922208 | 1.4475223 | 1.3907024 |
| Phase-1 RCT-290 | 1.0325769 | 1.1051288 | 0.9045534 | 1.3600566 | 1.1024619 | 0.9654473 |
| Transitional endoplasmic reticulum ATPase | 0.8119853 | 0.8339627 | 0.7378725 | 0.7803044 | 0.683457 | 0.7242665 |
| Beta-actin | 0.6619132 | 0.5855487 | 0.50707 | 0.6291993 | 0.4605782 | 0.4222727 |
| Thioredoxin-1 (Trx1) | 0.8456693 | 0.7747933 | 0.9031119 | 0.9422733 | 0.8682089 | 0.8527157 |
| Phase-1 RCT-174 | 0.9506312 | 0.9919269 | 1.007697 | 0.9635081 | 0.9861503 | 1.0293441 |
| Beta-actin, sequence 2 | 1.0977262 | 0.9190874 | 0.9396182 | 1.1354617 | 0.8480184 | 0.8001689 |
| Phase-1 RCT-109 | 1.0490971 | 1.0361731 | 1.1718973 | 1.2314739 | 1.1811662 | 1.027188 |
| Phase-1 RCT-154 | 1.019525 | 0.9633005 | 1.0217227 | 1.2451985 | 1.0335827 | 1.1074816 |
| Hypoxanthine-guanine | 0.8997778 | 0.8617771 | 0.838232 | 1.009886 | 0.9023998 | 0.866088 |
| phosphoribosyltransferase | | | | | | |
| Phase-1 RCT-213 | 1.0579875 | 0.9639002 | 0.9488586 | 0.994472 | 0.9454624 | 0.9607037 |
| Phase-1 RCT-260 | 0.9485223 | 1.0285974 | 1.0200483 | 0.974081 | 1.0404209 | 1.0294106 |
| Phase-1 RCT-12 | 1.0749242 | 1.1955858 | 1.1723201 | 1.2999413 | 1.0430565 | 1.1001389 |
| Phase-1 RCT-293 | 0.7238581 | 0.7731156 | 0.8844661 | 0.7916715 | 0.8300292 | 0.8399776 |
| Ribosomal protein S8 | 0.8894264 | 0.7598106 | 0.836594 | 0.9259967 | 0.7863433 | 0.8520291 |
| Pyruvate kinase, muscle | 0.6475626 | 0.7869531 | 0.9072191 | 0.741921 | 0.7310942 | 0.6376267 |
| Nucleoside diphosphate kinase beta isoform | 0.9999408 | 0.9277206 | 0.9048863 | 1.0934058 | 1.0649682 | 1.0173595 |
| p55CDC | 1.095358 | 1.0924687 | 1.0393854 | 0.8571633 | 1.0650216 | 1.1131505 |
| Phase-1 RCT-156 | 0.9820789 | 0.923961 | 0.9336618 | 1.0422528 | 0.9607407 | 0.9253122 |
| Ribosomal protein L13A | 1.0341544 | 1.0566301 | 1.1029173 | 1.2782404 | 1.1156323 | 1.0607251 |
| Phase-1 RCT-258 | 1.0336838 | 0.9713649 | 0.9734281 | 1.0544912 | 0.9571176 | 0.9885463 |
| Insulin-like growth factor I | 0.8867726 | 0.8975891 | 0.9843528 | 1.0496582 | 0.7559514 | 0.8419566 |
| Cytochrome P450 3A1 | 0.6074691 | 0.2627881 | 0.9259898 | 0.7426076 | 0.6627016 | 0.692426 |
| {Ribosomal protein 16} | 0.9129651 | 0.8736293 | 0.9020029 | 0.9734075 | 0.8585119 | 0.9200934 |
| Organic cation transporter 3 | 0.9058525 | 0.9200178 | 0.9396762 | 0.8637666 | 0.8576639 | 0.8247593 |
| Calpain 2 | 0.9354279 | 0.830666 | 0.9250379 | 0.8858175 | 0.8997908 | 0.958073 |
| Phase-1 RCT-102 | 0.7452305 | 0.715695 | 0.9176815 | 0.9329143 | 0.6189949 | 0.8084213 |
| Equilbrative nitrobenzylthioinosine-sensitive | 0.8760834 | 0.6067709 | 0.9293877 | 0.9483846 | 0.7854004 | 0.8127988 |
| nucleoside transporter | | | | | | |
| Phase-1 RCT-227 | 0.8452017 | 0.6879952 | 0.7648892 | 0.7551345 | 0.6446278 | 0.7441726 |
| N-hydroxy-2-acetylaminofluorene sulfotransferase (ST1C1) | 0.7281053 | 0.4432393 | 0.8887842 | 0.82286 | 0.6327095 | 0.7273149 |
| Cathepsin S | 0.7789674 | 0.7910013 | 0.87187 | 0.7136519 | 0.8181002 | 0.7940272 |
| Apolipoprotein CIII | 0.8670763 | 0.7750191 | 0.8804393 | 0.9071093 | 0.8639609 | 0.7635312 |
| Cytochrome P450 2A3 | 0.9827328 | 1.1304233 | 0.8856746 | 0.9418672 | 0.981898 | 1.0172508 |
| Cytochrome P450 2C23 | 0.9366972 | 0.9836605 | 0.7014574 | 0.9870718 | 0.9373009 | 0.8668826 |
| Apolipoprotein C1 | 0.7063373 | 0.7624822 | 0.6828909 | 0.8233021 | 0.9432644 | 0.7406676 |
| Betaine homocysteine methyltransferase (BHMT) | 1.1479098 | 1.0800139 | 0.7808873 | 1.8948418 | 1.1372718 | 0.8495852 |
| Paraoxonase 1 | 0.707365 | 0.6792337 | 0.6259528 | 0.6759706 | 0.6506668 | 0.6122415 |
| Phase-1 RCT-207 | 0.9327527 | 0.9542062 | 0.9952722 | 0.9452741 | 0.8719889 | 0.880065 |
| Cathepsin B | 0.956978 | 0.9188795 | 0.8879062 | 0.9091716 | 1.1465163 | 0.8362271 |
| Phase-1 RCT-144 | 0.9422835 | 1.3572382 | 0.9998572 | 0.8841462 | 0.8861185 | 1.0560048 |
| Alpha-1 microglobulin/bikunin precursor (Ambp) | 1.0374234 | 0.9097115 | 0.9424318 | 0.9742202 | 0.939552 | 0.903801 |
| Cyclin D1 | 0.7035454 | 0.8343072 | 1.14126 | 0.6856369 | 0.589191 | 0.9230226 |
| Presenilin-1 | 0.8581215 | 0.8911779 | 0.7849398 | 0.3221889 | 0.9363553 | 0.7913625 |
| Protein tyrosine phosphatase, receptor type, D | 1.0696431 | 0.852864 | 1.0990187 | 1.0684397 | 1.0076797 | 0.8182588 |
| Multidrug resistant protein-1 | 0.9176636 | 1.0657707 | 1.0486104 | 0.9916098 | 0.9714757 | 1.1281227 |
| Cathepsin L, sequence 2 | 1.0332272 | 1.1329306 | 1.0884858 | 1.2427462 | 1.209449 | 1.0499924 |
| JNK1 stress activated protein kinase | 1.0188972 | 1.1004592 | 0.9271039 | 1.1132449 | 1.2010175 | 0.9341255 |
| Phase-1 RCT-43 | 1.0157199 | 1.0228528 | 1.0259306 | 1.1136147 | 0.9998307 | 0.8720063 |
| Tissue plasminogen activator | 0.9744575 | 0.9928303 | 1.0137897 | 1.0397718 | 0.9964039 | 0.9993222 |
| Ubiquitin conjugating enzyme (RAD 6 | 0.9731624 | 0.9687661 | 1.0050666 | 0.8187173 | 0.9174989 | 0.9115771 |
| homologue) | | | | | | |
| Phase-1 RCT-173 | 1.0390466 | 0.8508195 | 1.061977 | 0.9899864 | 1.0957845 | 0.9550176 |
| Ornithine decarboxylase | 1.1596043 | 1.3319784 | 1.1275834 | 0.986988 | 1.053503 | 1.0835971 |
| Zinc finger protein | 1.0489175 | 0.9655274 | 0.9345885 | 1.0335797 | 1.095715 | 0.9780173 |
| CDK108 | 0.8735575 | 0.7728984 | 1.1875488 | 1.1439147 | 0.9134342 | 1.1014509 |
| Phase-1 RCT-117 | 0.9271666 | 0.7510801 | 0.7196028 | 1.0301169 | 0.8816976 | 0.9008879 |
| {Phase-1 RCT 98} | 1.007516 | 0.9938309 | 0.9800997 | 0.9369493 | 1.009007 | 1.0517182 |
| Aquaporin-3 (AQP3) | 0.928153 | 0.9089907 | 0.9218355 | 0.8886746 | 0.9215233 | 1.0280287 |
| Cholesterol esterase | 0.7044753 | 0.5261908 | 0.694319 | 0.7069235 | 0.6269354 | 0.7485949 |
| Ribosomal protein S17 | 0.9332551 | 0.8484786 | 0.835553 | 1.0126443 | 0.8995363 | 0.8791899 |
| Poly(ADP-ribose) polymerase | 1.060549 | 1.0054917 | 0.9130634 | 0.9984202 | 0.9887846 | 0.987048 |
| Annexin V | 0.8947868 | 0.8525893 | 0.8623068 | 0.7916028 | 0.7947308 | 0.9020819 |
| Phase-1 RCT-33 | 0.9710714 | 1.0277582 | 0.7743907 | 1.5780809 | 1.1403052 | 1.0328968 |
| Phase-1 RCT-61 | 0.9582841 | 1.020943 | 1.0148237 | 1.0630497 | 0.9847362 | 0.9493533 |
| Retinol dehydrogenase type III | 0.9683572 | 0.9003831 | 1.2663201 | 1.2804768 | 1.1973193 | 1.073542 |
| ATPase inhibitor (rat mitochondrial IF1 protein) | 0.7607908 | 0.615904 | 0.8028833 | 0.9046512 | 0.7828869 | 0.7260789 |
| Thymidylate synthase | 1.0580801 | 0.9866826 | 1.052848 | 0.9295431 | 1.0619593 | 1.0683392 |
| Interleukin-18 | 1.0688611 | 1.0316687 | 1.0108312 | 0.8878772 | 1.1034151 | 1.1037163 |
| Lecithin: cholesterol acyltransferase | 1.0344454 | 1.0954385 | 1.0680887 | 1.3465626 | 1.0573995 | 1.2002894 |
| Contrapsin-like protease inhibitor (CPi-21) | 1.040957 | 1.0822784 | 1.3116168 | 1.2504214 | 1.2221587 | 1.0620314 |
| Proliferating cell nuclear antigen gene | 0.9705562 | 0.9651532 | 0.9481137 | 0.8768196 | 1.0670022 | 1.0276155 |
| Phase-1 RCT-230 | 0.978582 | 1.1082714 | 1.028396 | 1.116379 | 1.1287479 | 1.0906312 |
| Cytochrome P450 2D18 | 1.0396953 | 1.0168067 | 1.1710697 | 1.3653011 | 1.071593 | 0.8795382 |
| Phase-1 RCT-48 | 1.0902524 | 1.0382491 | 1.2664733 | 1.1952866 | 1.0464191 | 0.9111628 |
| Phase-1 RCT-292 | 0.9404624 | 0.9964328 | 1.0369449 | 1.0012602 | 0.9479887 | 1.1120521 |
| Arginosuccinate synthetase 1 | 1.1266195 | 1.3714807 | 1.09294 | 1.9927161 | 1.3875583 | 1.0181397 |
| C-reactive protein | 1.0920905 | 0.8748298 | 1.1222543 | 1.0523262 | 1.0521997 | 0.8258616 |

| | KETO | KETO | KETO | KETO | KETO | KETO | LPS |
|---|---|---|---|---|---|---|---|
| Compound-Dose | 20 | 20 | 20 | 80 | 80 | 80 | 2 |
| Animal Number | 2227 | 2228 | 2229 | 2237 | 2238 | 2239 | 347 |
| Liver Toxicity Necrosis Classification | no | no | no | no | no | no | no |
| Gene Name | | | | | | | |
| Osteoactivin | 1.0618103 | 1.1725651 | 0.9697954 | 1.0082295 | 1.0405092 | 0.9704399 | 1.1499738 |
| Stathmin | 1.0357677 | 1.0036066 | 0.9932861 | 0.9886286 | 0.9606619 | 1.1128814 | 0.9267383 |
| Calpactin I heavy chain | 0.9951863 | 0.9299306 | 1.063261 | 1.0167985 | 1.0734273 | 1.0966058 | 1.0108827 |
| Phase-1 RCT-179 | 1.0972673 | 1.0847617 | 0.8997114 | 0.8990919 | 0.903918 | 0.8255838 | 1.1276257 |
| Phase-1 RCT-107 | 0.6416078 | 1.0320272 | 1.1648693 | 0.7578985 | 1.0401232 | 0.7761124 | 0.8659257 |
| Heme oxygenase | 1.0053705 | 1.2504977 | 0.9827848 | 1.0163479 | 0.803906 | 1.2070799 | 0.8551037 |
| Collagen type II | 1.2328186 | 0.7751147 | 0.8523824 | 1.1090308 | 0.9010714 | 1.2397212 | 0.7375642 |
| Phase-1 RCT-81 | 1.0603262 | 1.0843357 | 0.9960311 | 1.0407624 | 1.0110196 | 0.8786756 | 1.2623347 |
| Glycine methyltransferase | 0.5330267 | 0.9902627 | 1.1902028 | 0.6897563 | 1.0768687 | 0.6140596 | 0.3518358 |
| Cystatin C | 0.9925373 | 0.9827725 | 0.8428894 | 1.0008019 | 0.9143026 | 0.9128352 | 1.1215786 |
| Melanoma-associated antigen ME491 | 0.9610675 | 1.9187886 | 1.1822474 | 1.043293 | 0.9894872 | 1.0623997 | 0.9855518 |
| Phase-1 RCT-146 | 0.9290537 | 1.0654278 | 1.0088149 | 1.0845692 | 0.981095 | 1.3571917 | 0.8274188 |
| Gamma-actin, cytoplasmic | 0.8008093 | 0.8682196 | 0.7539828 | 0.9713573 | 1.246067 | 1.0352285 | 1.009393 |
| Adenine nucleotide translocator 1 | 1.147787 | 0.8575854 | 0.923081 | 0.9912056 | 1.0200942 | 0.9158759 | 0.8362757 |
| Thymosin beta-10 | 0.9174476 | 0.9673879 | 0.9273673 | 0.8716582 | 0.8310276 | 0.9993249 | 1.3834974 |
| Insulin-like growth factor I, exon 6 | 1.3872732 | 0.833457 | 1.0006926 | 1.0401957 | 1.1763947 | 1.1168411 | 0.6370295 |
| Phase-1 RCT-78 | 1.0681082 | 1.1640416 | 1.0239267 | 0.9812399 | 1.0582012 | 1.0116067 | 0.9913063 |
| Alpha-2-microglobulin | 1.0180439 | 0.9472672 | 1.1240027 | 1.0798064 | 1.2537565 | 1.0108689 | 0.7191268 |
| High affinity IgE receptor gamma chain | 1.1363595 | 1.0432348 | 1.0214124 | 1.0696034 | 1.0528743 | 1.118954 | 1.5133718 |
| (FcERIgamma) | | | | | | | |
| 60S ribosomal protein L6 | 1.1487309 | 1.0746465 | 0.8424394 | 1.0485139 | 0.923162 | 0.8119853 | 1.2581455 |
| IgE binding protein | 1.0012897 | 1.0960099 | 1.1123488 | 1.0677552 | 1.0173423 | 1.2354007 | 1.2629712 |
| Phase-1 RCT-149 | 0.9293258 | 0.926046 | 1.061144 | 1.0044552 | 1.056926 | 1.003527 | 1.106527 |
| Cytochrome P450 2C11 | 1.550108 | 0.7128838 | 1.3732378 | 1.2340543 | 1.4566 | 1.2789942 | 0.9747297 |
| Uncoupling protein 2 | 0.8807271 | 1.0007331 | 0.9286529 | 0.9419692 | 0.8775904 | 0.9863997 | 1.1124645 |
| Cofilin | 1.1469418 | 0.9454505 | 0.9915666 | 1.110541 | 1.0722455 | 0.8310072 | 1.5354941 |
| Alpha-tubulin | 0.9593689 | 0.8346862 | 0.8314908 | 0.996404 | 1.1238962 | 0.9830285 | 0.7473927 |
| Stearyl-CoA desaturase, liver | 2.7434795 | 0.0528774 | 1.5265566 | 0.8228114 | 2.0229478 | 2.0737667 | 0.8492199 |
| Glutathione S-transferase theta-1 | 0.878396 | 0.4983854 | 0.6366832 | 0.6578221 | 1.2326202 | 0.725576 | 1.0794172 |
| alpha-1,2-fucosyltrans-ferase | 1.130649 | 0.9989219 | 1.0255107 | 1.0964191 | 1.048554 | 0.9830108 | 1.227629 |
| Phase-1 RCT-138 | 0.9866093 | 1.0000538 | 0.9914349 | 1.0105298 | 0.9274696 | 0.9257351 | 1.0638621 |
| Phase-1 RCT-295 | 1.2119931 | 1.0758541 | 1.0999397 | 1.0738385 | 1.0942107 | 1.0732532 | 1.3107479 |
| Phase-1 RCT-164 | 1.1685184 | 1.0252593 | 0.9875138 | 1.0063412 | 0.9972071 | 0.9020976 | 1.1018918 |
| Alpha-2-macroglobulin, sequence 2 | 0.9042392 | 1.1605289 | 0.9442838 | 1.0258551 | 1.0423769 | 1.0519522 | 1.1920803 |
| Biliverdin reductase | 0.9538349 | 1.0320388 | 1.0425723 | 1.0212481 | 0.9413747 | 1.1064318 | 0.8140081 |
| Phase-1 RCT-121 | 0.8627341 | 1.0812109 | 0.9921852 | 0.8820637 | 0.9134206 | 1.0411897 | 0.7225234 |
| Dynamin-1 (D100) | 1.0924703 | 0.9686557 | 0.9748409 | 0.9588119 | 1.1066854 | 0.8945246 | 1.2370943 |
| Phase-1 RCT-192 | 1.3018655 | 0.9023318 | 0.8504744 | 1.0238671 | 0.9010121 | 0.8411636 | 0.8486555 |
| Fatty acid synthase | 1.0730834 | 0.4076232 | 1.1922983 | 0.8384709 | 1.7206244 | 1.6078854 | 0.9166868 |
| Phase-1 RCT-211 | 0.9487587 | 1.024401 | 1.2431078 | 1.1254956 | 1.0557357 | 1.221727 | 1.1237944 |
| Ribosomal protein S9 | 1.06663 | 1.1360338 | 0.9508738 | 1.1176602 | 0.9088898 | 0.8330949 | 1.3565247 |
| NADP-dependent isocitrate dehydrogenase, | 1.2135843 | 0.8221809 | 0.8830767 | 0.985211 | 0.8837373 | 0.84346 | 1.0050313 |
| cytosolic | | | | | | | |
| Phase-1 RCT-57 | 0.7791845 | 1.2377871 | 0.9214695 | 0.814515 | 0.9481741 | 0.7681946 | 0.8724057 |
| Phase-1 RCT-36 | 0.9839906 | 0.9711403 | 1.0382029 | 0.9388719 | 0.892493 | 0.9964589 | 1.0290487 |
| Apolipoprotein All | 1.06563 | 0.936967 | 0.9513488 | 0.9936955 | 1.0165778 | 0.6444051 | 1.2653373 |
| Phase-1 RCT-68 | 1.045464 | 1.0570312 | 1.0127146 | 0.9530968 | 1.0354431 | 0.9659874 | 1.0540302 |
| Multidrug resistant protein-2 | 0.6855845 | 1.0431606 | 0.8054094 | 0.8314571 | 0.7792258 | 0.8768979 | 0.7489455 |
| Phase-1 RCT-39 | 0.9899807 | 1.0758013 | 1.0098232 | 0.9274405 | 0.9616258 | 1.0539874 | 0.7752257 |
| NIPK | 1.1596767 | 1.0379992 | 0.9947108 | 0.9706942 | 1.0612465 | 1.1936558 | 0.7837969 |
| F1-ATPase beta subunit | 1.3007109 | 1.0091318 | 1.0411857 | 1.0776129 | 1.124045 | 0.8868392 | 1.1212081 |
| Elongation factor-1 alpha | 0.9939349 | 1.0491428 | 0.8125841 | 0.8352483 | 0.8972975 | 0.6678699 | 1.3254089 |
| Phase-1 RCT-24 | 1.1246889 | 0.7830836 | 0.8442555 | 1.0721177 | 1.2694421 | 1.1398975 | 1.1975793 |
| Beta-tubulin, class I | 1.1831266 | 0.7317823 | 0.804827 | 1.1201948 | 1.3746914 | 1.1141939 | 1.4380772 |
| 14-3-3 zeta | 1.0667069 | 1.0059623 | 0.9059894 | 0.9077148 | 0.9943005 | 1.006612 | 0.7201351 |
| Alpha-prothymosin | 1.1920507 | 0.9250034 | 0.9334533 | 1.0602723 | 1.0084984 | 0.8400812 | 1.4921767 |
| Phase-1 RCT-58 | 1.0518457 | 0.7900956 | 0.8028967 | 0.7379812 | 0.881271 | 1.0563208 | 0.7222381 |
| Phase-1 RCT-290 | 1.0619867 | 0.6914315 | 1.6789665 | 1.4707156 | 1.8079067 | 1.2459372 | 1.2137754 |
| Transitional endoplasmic reticulum ATPase | 0.9009858 | 0.928995 | 0.8757679 | 0.9129844 | 1.0203118 | 0.8473463 | 0.9245597 |
| Beta-actin | 0.8893366 | 0.8629075 | 1.027671 | 0.6894912 | 1.287639 | 1.0513736 | 0.8041112 |
| Thioredoxin-1 (Trx1) | 1.2202142 | 1.013655 | 1.0596806 | 1.1403744 | 1.1277674 | 1.1090256 | 1.1850754 |
| Phase-1 RCT-174 | 1.1679206 | 0.965239 | 1.088437 | 1.0718503 | 1.1508725 | 1.0694553 | 1.3054378 |
| Beta-actin, sequence 2 | 1.0602272 | 0.946383 | 0.9996186 | 1.0283955 | 1.2066067 | 1.0264846 | 1.2887661 |
| Phase-1 RCT-109 | 0.9442712 | 1.0646056 | 0.8871577 | 0.9239832 | 0.8651074 | 0.8263656 | 1.3273343 |
| Phase-1 RCT-154 | 1.0685546 | 1.0149292 | 0.955093 | 1.0831414 | 0.9316033 | 0.9810329 | 0.9040819 |
| Hypoxanthine-guanine | 1.1129258 | 0.9299717 | 1.0987909 | 0.9387038 | 1.0729774 | 0.9862529 | 0.8423265 |
| phosphoribosyltransferase | | | | | | | |
| Phase-1 RCT-213 | 0.976692 | 1.034677 | 0.9556306 | 1.0234357 | 1.0269551 | 1.0714388 | 0.8313937 |
| Phase-1 RCT-260 | 0.96 | 1.0696896 | 1.2236644 | 1.0790672 | 1.0238128 | 1.2142787 | 0.9627603 |
| Phase-1 RCT-12 | 0.928461 | 0.8764984 | 0.960214 | 0.9017626 | 1.0820802 | 1.0643618 | 1.2625679 |
| Phase-1 RCT-293 | 1.0460149 | 0.9762187 | 0.9702634 | 1.033324 | 1.0379231 | 1.0188287 | 1.33934 |
| Ribosomal protein S8 | 1.1908447 | 1.0642874 | 0.8912666 | 1.0678409 | 0.9872188 | 0.8573498 | 2.0813603 |
| Pyruvate kinase, muscle | 0.9945719 | 1.0039778 | 0.9607332 | 0.9222968 | 0.981008 | 0.9397318 | 0.9281352 |
| Nucleoside diphosphate kinase beta isoform | 1.1740694 | 0.8725343 | 1.02956 | 1.0435872 | 1.0018204 | 1.119486 | 1.0417342 |
| p55CDC | 0.7804593 | 0.9875785 | 0.9299048 | 1.0152817 | 0.8544423 | 1.3350735 | 0.8550383 |
| Phase-1 RCT-156 | 0.9608464 | 0.9501404 | 0.9285223 | 0.8306595 | 0.8499646 | 0.8287804 | 1.1273481 |
| Ribosomal protein L13A | 0.9555394 | 1.0579706 | 0.8807319 | 0.8715127 | 0.8388116 | 0.7791645 | 1.0630088 |
| Phase-1 RCT-258 | 0.9913226 | 1.0292852 | 0.9529764 | 0.9912073 | 0.9860693 | 0.9814863 | 0.8075185 |
| Insulin-like growth factor I | 1.3885748 | 0.7124481 | 0.9073932 | 1.2899565 | 1.0668842 | 1.0686615 | 0.9199272 |
| Cytochrome P450 3A1 | 0.6792537 | 1.3783569 | 0.7842236 | 0.9582224 | 0.9869733 | 0.8145282 | 0.5481643 |
| {Ribosomal protein 16} | 1.1547095 | 1.0906253 | 0.9117827 | 1.0019065 | 1.0195365 | 0.873324 | 1.5547022 |
| Organic cation transporter 3 | 1.133396 | 0.9808955 | 0.7915407 | 1.0270245 | 0.9367722 | 0.7897837 | 1.2360339 |
| Calpain 2 | 1.0055411 | 1.0074106 | 1.0113428 | 0.9740971 | 0.9663036 | 1.0820388 | 1.1055788 |
| Phase-1 RCT-102 | 1.7152256 | 0.4570049 | 1.3626374 | 1.5261964 | 1.3880997 | 1.4555887 | 0.8361394 |
| Equilbrative nitrobenzylthioinosine-sensitive | 1.0084519 | 1.0556134 | 0.9961531 | 1.1150582 | 1.0521215 | 1.0672125 | 0.9397445 |
| nucleoside transporter | | | | | | | |
| Phase-1 RCT-227 | 1.1789032 | 1.5770384 | 0.9449762 | 0.9757962 | 1.0903181 | 0.8728708 | 1.0237017 |
| N-hydroxy-2-acetylaminofluorene sulfotransferase (ST1C1) | 1.0856069 | 1.0558393 | 0.8776359 | 1.1009021 | 1.0590694 | 1.0777491 | 0.9508981 |
| Cathepsin S | 0.9136758 | 0.9466658 | 0.909021 | 1.0471793 | 0.9397074 | 1.1021055 | 0.9767974 |
| Apolipoprotein CIII | 1.1074739 | 1.0116826 | 1.062951 | 0.9106597 | 0.9860711 | 0.8982792 | 1.0108827 |
| Cytochrome P450 2A3 | 1.1003094 | 1.0571331 | 0.8898748 | 1.161168 | 1.0691106 | 0.6863378 | 1.1598556 |
| Cytochrome P450 2C23 | 0.9939448 | 1.1111925 | 0.6322894 | 0.8846563 | 0.8464382 | 0.6708217 | 1.3627856 |
| Apolipoprotein C1 | 1.0450132 | 1.2988476 | 0.8815001 | 0.8763421 | 0.9254555 | 0.6731924 | 1.7300364 |
| Betaine homocysteine methyltransferase (BHMT) | 1.0489192 | 0.5996926 | 1.9307102 | 1.5501614 | 1.8862896 | 1.27551 | 1.405062 |
| Paraoxonase 1 | 1.1025195 | 1.0417995 | 0.8626052 | 1.0941684 | 0.9917534 | 0.7644303 | 1.704653 |
| Phase-1 RCT-207 | 0.8909985 | 1.0690686 | 0.9697805 | 0.8851261 | 0.9768459 | 1.0299163 | 0.7964164 |
| Cathepsin B | 0.9808558 | 0.8981602 | 0.8859205 | 0.8679422 | 0.8338279 | 0.8411909 | 1.253539 |
| Phase-1 RCT-144 | 1.1832355 | 0.9952699 | 0.9862852 | 0.9659624 | 0.9450122 | 1.1100124 | 1.102073 |
| Alpha-1 microglobulin/bikunin precursor (Ambp) | 1.1227962 | 1.0573705 | 0.966149 | 1.0744686 | 1.0001813 | 0.7840695 | 1.4693968 |
| Cyclin D1 | 1.123014 | 0.7746915 | 0.8279488 | 1.0960695 | 1.101492 | 1.1195472 | 0.7388634 |
| Presenilin-1 | 0.9684903 | 1.0257176 | 0.9249352 | 0.9730316 | 0.8555638 | 0.8276807 | 1.2522312 |
| Protein tyrosine phosphatase, receptor type, D | 1.2337748 | 0.7944145 | 1.0954939 | 1.0424148 | 1.1221355 | 1.0275847 | 1.327915 |
| Multidrug resistant protein-1 | 0.4633577 | 1.3605262 | 1.1036029 | 0.8336357 | 0.8473235 | 0.8823658 | 0.8641516 |
| Cathepsin L, sequence 2 | 0.7435958 | 1.1466589 | 0.6609684 | 0.8571227 | 0.780852 | 0.7177197 | 0.9437895 |
| JNK1 stress activated protein kinase | 0.9353724 | 0.9236269 | 1.0434995 | 1.0574105 | 1.1520783 | 0.8086606 | 1.0917792 |
| Phase-1 RCT-43 | 0.9611611 | 0.9559153 | 0.9245868 | 0.8882048 | 0.8983784 | 0.8222454 | 0.8323638 |
| Tissue plasminogen activator | 1.0537682 | 0.9543054 | 1.0671182 | 1.0410353 | 1.0798628 | 1.0878012 | 0.9901866 |
| Ubiquitin conjugating enzyme (RAD 6 | 1.0779992 | 1.0539842 | 0.8922252 | 1.0918648 | 0.9169959 | 0.8495632 | 1.4173315 |
| homologue) | | | | | | | |
| Phase-1 RCT-173 | 0.7316627 | 1.1358261 | 0.9940911 | 0.9824165 | 1.064026 | 1.2300069 | 0.7764348 |
| Ornithine decarboxylase | 1.0218287 | 0.956551 | 1.0639799 | 0.9476331 | 1.1777921 | 1.0983492 | 0.6262221 |
| Zinc finger protein | 0.892592 | 1.2930791 | 0.9041849 | 0.957519 | 0.8497521 | 0.9576426 | 0.8343093 |
| CDK108 | 0.9188712 | 1.0944127 | 1.0891309 | 0.8885934 | 1.2916408 | 1.3232597 | 0.8838292 |
| Phase-1 RCT-117 | 1.1182582 | 1.0109799 | 1.1137104 | 0.98249 | 1.0622932 | 1.0357401 | 1.0376747 |
| {Phase-1 RCT 98} | 0.9941011 | 0.966181 | 1.0519491 | 1.0575081 | 1.1521331 | 0.9899253 | 0.9943522 |
| Aquaporin-3 (AQP3) | 0.9837914 | 0.9868083 | 1.0026027 | 0.9955408 | 1.0201402 | 1.1444134 | 0.9534005 |
| Cholesterol esterase | 0.7807266 | 0.8090391 | 0.8367775 | 0.9474692 | 1.1836591 | 0.8412047 | 0.963367 |
| Ribosomal protein S17 | 1.1353065 | 1.1709368 | 0.8701046 | 1.082541 | 0.9565603 | 0.8999066 | 1.9184402 |
| Poly(ADP-ribose) polymerase | 0.9059508 | 0.9157959 | 0.9991729 | 1.0011454 | 1.029419 | 1.050837 | 0.9782824 |
| Annexin V | 1.2992718 | 0.9431049 | 0.7619942 | 1.1272126 | 1.0546576 | 0.9688961 | 0.8529686 |
| Phase-1 RCT-33 | 1.0611541 | 0.8695906 | 0.9619659 | 0.8851355 | 0.8146865 | 0.9320778 | 1.1280866 |
| Phase-1 RCT-61 | 0.9857454 | 0.9574078 | 0.9294578 | 0.8140184 | 0.9384996 | 1.127232 | 0.7485184 |
| Retinol dehydrogenase type III | 1.2986244 | 1.0775114 | 1.0760976 | 1.0367891 | 0.98343 | 0.8922851 | 1.6817278 |
| ATPase inhibitor (rat mitochondrial IF1 protein) | 1.2283452 | 0.9106761 | 0.8806968 | 1.1302642 | 1.0251763 | 0.938646 | 2.222958 |
| Thymidylate synthase | 0.8448799 | 0.9882188 | 0.9772775 | 0.8694392 | 0.9340508 | 1.0758566 | 0.8974919 |
| Interleukin-18 | 0.9310843 | 1.6132858 | 0.9736366 | 1.0460393 | 0.9327579 | 0.98555 | 0.885281 |
| Lecithin: cholesterol acyltransferase | 1.2097968 | 1.1212023 | 0.9220008 | 1.1214192 | 0.9355801 | 0.8285992 | 1.2235104 |
| Contrapsin-like protease inhibitor (CPi-21) | 1.2392895 | 1.5013045 | 1.1838784 | 1.1539097 | 1.0824828 | 0.9331396 | 1.014031 |
| Proliferating cell nuclear antigen gene | 1.0018058 | 1.0789398 | 0.9441286 | 0.960838 | 0.9179581 | 0.9986333 | 0.7836894 |
| Phase-1 RCT-230 | 0.956540 | 1.1562042 | 1.1512678 | 1.0514365 | 1.0404675 | 1.3736684 | 0.7287259 |
| Cytochrome P450 2D18 | 0.7392199 | 1.3668108 | 0.9729657 | 0.8160653 | 0.9510136 | 0.7026509 | 0.9402359 |
| Phase-1 RCT-48 | 1.2375287 | 0.7293276 | 1.1475164 | 1.0885278 | 1.0595316 | 1.2361956 | 0.6803766 |
| Phase-1 RCT-292 | 0.9300364 | 0.9459599 | 0.9800494 | 0.9413398 | 0.921587 | 1.0440986 | 0.9189843 |
| Arginosuccinate synthetase 1 | 1.086732 | 1.1886728 | 1.4094157 | 0.8552293 | 0.9643988 | 0.7340687 | 1.5042547 |
| C-reactive protein | 1.0874767 | 0.7270421 | 1.0439742 | 0.9154378 | 0.9665421 | 0.8273908 | 1.5559416 |

| | LPS | LPS | LPS | LPS | MET | NAL |
|---|---|---|---|---|---|---|
| Compound-Dose | 2 | 2 | 8 | 8 | 5 | 45 |
| Animal Number | 348 | 349 | 357 | 358 | 238 | 2647 |
| Liver Toxicity Necrosis Classification | no | no | yes | yes | no | no |
| Gene Name | | | | | | |
| Osteoactivin | 1.1109719 | 1.073597 | 1.6118702 | 1.4074453 | 0.8607398 | 0.9978581 |
| Stathmin | 1.1438521 | 1.0191717 | 1.35503361 | 1.2449396 | 0.931918 | 0.8905404 |
| Calpactin I heavy chain | 0.9878622 | 1.1768284 | 1.0598588 | 1.9322118 | 1.1698265 | 1.0355558 |
| Phase-1 RCT-179 | 1.0826172 | 1.0281196 | 1.2718953 | 1.1947907 | 0.7719986 | 0.9788885 |
| Phase-1 RCT-107 | 1.6675018 | 1.318598 | 0.7016642 | 0.6188124 | 1.094159 | 0.8031592 |
| Heme oxygenase | 1.2530507 | 1.032837 | 1.2870758 | 3.4576108 | 0.9956706 | 1.008816 |
| Collagen type II | 0.8323454 | 1.3210893 | 1.7454416 | 1.0433245 | 1.7413026 | 0.6877955 |
| Phase-1 RCT-81 | 0.8362652 | 0.9299761 | 1.0545126 | 0.728508 | 1.0568056 | 1.2989204 |
| Glycine methyltransferase | 2.5294402 | 1.966606 | 1.2850665 | 0.1432965 | 0.9882745 | 0.754256 |
| Cystatin C | 0.9016414 | 0.9553244 | 0.8600019 | 0.5145915 | 0.8542857 | 1.1083809 |
| Melanoma-associated antigen ME491 | 1.1614159 | 1.1374 | 0.8429529 | 2.057486 | 1.1153917 | 1.0218906 |
| Phase-1 RCT-146 | 1.1850945 | 1.1219636 | 1.098419 | 1.2703335 | 1.1556522 | 0.8689675 |
| Gamma-actin, cytoplasmic | 0.4760669 | 0.7684445 | 1.2823808 | 2.0532982 | 1.112652 | 0.9582595 |
| Adenine nucleotide translocator 1 | 0.6473575 | 0.85789 | 1.0828714 | 0.8819037 | 0.9250249 | 0.768063 |
| Thymosin beta-10 | 1.3242525 | 1.1440005 | 1.5212133 | 2.4276903 | 0.8060554 | 1.0036772 |
| Insulin-like growth factor I, exon 6 | 1.1221433 | 0.7535741 | 0.8583984 | 0.6595183 | 0.7295475 | 0.9542673 |
| Phase-1 RCT-78 | 0.7771417 | 0.7548003 | 0.837193 | 0.5694442 | 0.9653873 | 1.2268869 |
| Alpha-2-microglobulin | 0.7692493 | 0.538294 | 0.2616363 | 0.5252923 | 0.9352568 | 1.1238519 |
| High affinity IgE receptor gamma chain | 0.9459187 | 1.1436844 | 1.6383197 | 1.4425371 | 0.7992657 | 1.1030726 |
| (FcERIgamma) | | | | | | |
| 60S ribosomal protein L6 | 1.1350762 | 1.0547867 | 1.4829026 | 1.3535442 | 0.8559651 | 1.0567036 |
| IgE binding protein | 1.2220999 | 1.3936443 | 1.4251382 | 3.767226 | 0.8553075 | 0.8920017 |
| Phase-1 RCT-149 | 0.923404 | 1.0618299 | 0.9866853 | 3.9644027 | 1.2375284 | 1.0483816 |
| Cytochrome P450 2C11 | 1.0719885 | 0.7636187 | 0.5101882 | 0.2999374 | 2.4186213 | 0.7637638 |
| Uncoupling protein 2 | 1.1466694 | 1.2996688 | 1.8968482 | 1.3210992 | 1.123848 | 0.904999 |
| Cofilin | 0.8792518 | 1.0044409 | 1.2885987 | 0.8125472 | 0.8003139 | 1.2091295 |
| Alpha-tubulin | 0.9184218 | 0.816904 | 1.0560966 | 1.1096114 | 1.5741191 | 1.1181636 |
| Stearyl-CoA desaturase, liver | 0.1381182 | 0.4019637 | 0.0475301 | 0.0437714 | 1.3993397 | 0.4787742 |
| Glutathione S-transferase theta-1 | 0.8780777 | 1.1614978 | 1.3185403 | 0.9390618 | 0.9622586 | 0.9556687 |
| alpha-1,2-fucosyltrans-ferase | 1.5305979 | 0.761538 | 0.9970174 | 0.7149516 | 1.1111975 | 0.8042939 |
| Phase-1 RCT-138 | 1.1386904 | 1.2640575 | 1.3631569 | 1.0008943 | 0.9911642 | 1.0084654 |
| Phase-1 RCT-295 | 1.04514 | 0.8641897 | 1.1506847 | 1.0590435 | 0.980996 | 1.0613744 |
| Phase-1 RCT-164 | 1.0118599 | 1.0524917 | 0.8985902 | 0.6595337 | 0.7532137 | 1.2652526 |
| Alpha-2-macroglobulin, sequence 2 | 0.9910222 | 1.1365511 | 1.100631 | 2.0252783 | 0.767556 | 0.7566994 |
| Biliverdin reductase | 1.0048004 | 1.1377785 | 1.4140321 | 0.9248817 | 1.6762749 | 0.9899457 |
| Phase-1 RCT-121 | 1.3386292 | 1.1164314 | 1.4963027 | 1.0293612 | 1.0499172 | 0.7506802 |
| Dynamin-1 (D100) | 1.1646252 | 0.9901801 | 0.9502544 | 0.6032047 | 0.8135103 | 1.0761161 |
| Phase-1 RCT-192 | 0.9528216 | 1.0256162 | 1.2221013 | 1.1908112 | 0.8426656 | 0.903061 |
| Fatty acid synthase | 0.6869329 | 0.49216 | 0.2547886 | 0.5412595 | 1.3090779 | 0.6754306 |
| Phase-1 RCT-211 | 1.3507557 | 0.8904139 | 0.8945967 | 1.6380981 | 0.9108986 | 0.8478723 |
| Ribosomal protein S9 | 0.9008379 | 0.9440257 | 1.3917673 | 1.2909693 | 1.3449265 | 1.0381054 |
| NADP-dependent isocitrate dehydrogenase, | 0.9289604 | 1.1501498 | 1.3217151 | 0.4991648 | 0.8656116 | 1.145767 |
| cytosolic | | | | | | |
| Phase-1 RCT-57 | 1.1133958 | 0.9240596 | 0.8361725 | 1.0721129 | 0.9394465 | 1.030092 |
| Phase-1 RCT-36 | 1.0555774 | 1.0488957 | 0.7718663 | 0.7520562 | 1.0121013 | 1.1452247 |
| Apolipoprotein All | 2.9859982 | 0.7418725 | 0.7688524 | 0.2823384 | 0.3940391 | 0.5854952 |
| Phase-1 RCT-68 | 1.0231626 | 1.0812482 | 1.2202263 | 2.1922286 | 1.0751817 | 1.0307137 |
| Multidrug resistant protein-2 | 0.9655973 | 1.1954582 | 1.8914113 | 1.0693926 | 1.8992503 | 0.8569303 |
| Phase-1 RCT-39 | 1.0325894 | 0.9881377 | 0.5564544 | 1.4656085 | 1.1778817 | 0.7822692 |
| NIPK | 1.0255058 | 0.967332 | 0.8806583 | 0.8719173 | 0.9490244 | 0.9137592 |
| F1-ATPase beta subunit | 0.6719763 | 0.8329393 | 0.9210616 | 0.578769 | 1.1389529 | 1.1662457 |
| Elongation factor-1 alpha | 0.9885654 | 1.0284476 | 1.5525622 | 1.0198519 | 1.5254289 | 1.0901872 |
| Phase-1 RCT-24 | 0.861874 | 0.8646196 | 1.070531 | 0.9617021 | 1.1443762 | 0.8488769 |
| Beta-tubulin, class I | 0.6872869 | 0.9894317 | 0.8950029 | 0.8110338 | 1.0295627 | 0.8574175 |
| 14-3-3 zeta | 0.9200957 | 0.900279 | 1.1444138 | 1.4321153 | 1.6451011 | 0.9203205 |
| Alpha-prothymosin | 0.8782797 | 0.7920702 | 1.2273427 | 0.5698307 | 0.9466316 | 1.3084159 |
| Phase-1 RCT-58 | 0.9636587 | 1.2072132 | 1.611833 | 0.8087139 | 0.7597046 | 0.5749461 |
| Phase-1 RCT-290 | 1.1844711 | 1.1639888 | 0.7738295 | 0.4114957 | 0.7509206 | 1.3822116 |
| Transitional endoplasmic reticulum ATPase | 0.6517322 | 0.9946396 | 1.075758 | 1.0174806 | 1.2180117 | 1.2043289 |
| Beta-actin | 0.7553344 | 0.7546243 | 1.5399238 | 1.4478053 | 1.6182171 | 0.8890583 |
| Thioredoxin-1 (Trx1) | 1.2211549 | 0.8254542 | 1.0360731 | 1.4207895 | 0.6153888 | 0.8781273 |
| Phase-1 RCT-174 | 0.7789581 | 1.0065122 | 0.8868034 | 0.8545728 | 1.0991783 | 1.0608683 |
| Beta-actin, sequence 2 | 1.4419211 | 0.9194955 | 1.3027158 | 1.8715502 | 0.6845053 | 1.1175635 |
| Phase-1 RCT-109 | 1.5537783 | 1.0129572 | 1.231006 | 2.0403097 | 0.7841685 | 0.9652577 |
| Phase-1 RCT-154 | 1.0656397 | 1.1508584 | 0.931163 | 1.3778303 | 1.1550199 | 1.0843228 |
| Hypoxanthine-guanine | 0.799869 | 0.8577602 | 1.1326075 | 1.0975078 | 1.4547588 | 1.046779 |
| phosphoribosyltransferase | | | | | | |
| Phase-1 RCT-213 | 1.0396932 | 0.9549944 | 0.9024361 | 1.0931168 | 1.0802186 | 0.8718408 |
| Phase-1 RCT-260 | 0.9653627 | 1.1308321 | 0.4468145 | 0.5744744 | 0.8040164 | 0.7951618 |
| Phase-1 RCT-12 | 0.9231458 | 1.074201 | 0.9099266 | 1.0985059 | 0.9297852 | 0.930748 |
| Phase-1 RCT-293 | 1.0748171 | 1.1238401 | 1.3600008 | 1.6220219 | 0.7931545 | 1.012319 |
| Ribosomal protein S8 | 1.1953193 | 1.0681615 | 1.3267509 | 1.5123247 | 0.6152434 | 1.0617156 |
| Pyruvate kinase, muscle | 1.1484793 | 1.0003225 | 0.633912 | 1.7183726 | 1.037163 | 1.0495372 |
| Nucleoside diphosphate kinase beta isoform | 0.73695 | 0.8210776 | 0.9680903 | 1.2260188 | 0.8109011 | 0.8240356 |
| p55CDC | 0.9202584 | 1.0079651 | 1.1542108 | 2.3778481 | 1.302827 | 0.8196846 |
| Phase-1 RCT-156 | 0.8525945 | 0.8657587 | 1.0327852 | 0.9654631 | 0.7867059 | 1.0131239 |
| Ribosomal protein L13A | 1.1194626 | 1.0256776 | 1.3632537 | 2.247025 | 1.2409654 | 0.9369394 |
| Phase-1 RCT-258 | 1.0541316 | 1.0248635 | 1.0059857 | 1.154545 | 1.150075 | 0.9107769 |
| Insulin-like growth factor I | 0.9220709 | 0.7746004 | 0.9229568 | 0.4386185 | 0.8528367 | 1.2433594 |
| Cytochrome P450 3A1 | 1.4796853 | 0.385293 | 0.3575777 | 0.0980983 | 1.0587738 | 1.0438898 |
| {Ribosomal protein 16} | 1.1464562 | 1.011876 | 1.2683167 | 1.5376393 | 0.6912049 | 1.0054084 |
| Organic cation transporter 3 | 1.0742307 | 1.0661566 | 0.7431863 | 1.1867591 | 1.1260966 | 1.0815742 |
| Calpain 2 | 0.940981 | 1.0116924 | 1.2158599 | 1.1343175 | 0.9907947 | 1.0199475 |
| Phase-1 RCT-102 | 0.7009646 | 0.6919818 | 0.5131805 | 0.2784272 | 0.8968893 | 1.0504878 |
| Equilbrative nitrobenzylthioinosine-sensitive | 0.8833206 | 0.8225213 | 0.4728195 | 0.4382838 | 0.8815355 | 0.973964 |
| nucleoside transporter | | | | | | |
| Phase-1 RCT-227 | 0.8119335 | 0.4496424 | 0.7767955 | 0.2953237 | 0.7718899 | 1.2624382 |
| N-hydroxy-2-acetylaminofluorene sulfotransferase (ST1C1) | 0.1876423 | 0.6438901 | 0.6330161 | 0.0858936 | 0.9790942 | 0.9841273 |
| Cathepsin S | 1.098088 | 1.0983486 | 1.3528031 | 1.0384243 | 1.0561284 | 1.3037747 |
| Apolipoprotein CIII | 1.2406608 | 0.9526882 | 1.0805821 | 1.1166836 | 1.2818261 | 1.2675391 |
| Cytochrome P450 2A3 | 1.0937663 | 1.1663631 | 0.8573025 | 0.4972604 | 1.2050781 | 1.3215362 |
| Cytochrome P450 2C23 | 0.9084136 | 1.2304703 | 1.6954662 | 0.173472 | 0.682736 | 0.9890165 |
| Apolipoprotein C1 | 0.795972 | 0.6529711 | 1.071679 | 0.265836 | 0.5117769 | 1.3140316 |
| Betaine homocysteine methyltransferase (BHMT) | 1.3421605 | 1.282184 | 0.6906168 | 0.0712525 | 0.576084 | 1.4516473 |
| Paraoxonase 1 | 0.8922243 | 1.022918 | 1.3272403 | 0.1367027 | 0.9368402 | 1.1585777 |
| Phase-1 RCT-207 | 1.1617899 | 0.9963223 | 0.8915475 | 1.0947728 | 1.1096212 | 0.8653039 |
| Cathepsin B | 0.8981218 | 0.981882 | 1.3886065 | 1.7867509 | 1.185339 | 1.1550395 |
| Phase-1 RCT-144 | 0.9002206 | 0.9280213 | 1.1453855 | 1.3175316 | 1.1061742 | 1.0920479 |
| Alpha-1 microglobulin/bikunin precursor (Ambp) | 0.7547584 | 0.8745387 | 1.2435149 | 0.7159473 | 0.917112 | 1.3791671 |
| Cyclin D1 | 1.4721022 | 0.5383818 | 1.0256747 | 0.6498021 | 1.5486716 | 1.2139559 |
| Presenilin-1 | 0.946695 | 0.9551008 | 1.0025127 | 0.4169594 | 0.931981 | 1.1816046 |
| Protein tyrosine phosphatase, receptor type, D | 0.6696814 | 1.0260677 | 1.1157317 | 0.7172641 | 0.6381334 | 1.1097333 |
| Multidrug resistant protein-1 | 0.8202049 | 1.1339741 | 2.0160375 | 0.9582012 | 1.5876356 | 0.9688236 |
| Cathepsin L, sequence 2 | 1.1781821 | 1.2811123 | 1.6058435 | 1.8552697 | 0.8922631 | 1.1261553 |
| JNK1 stress activated protein kinase | 1.2398584 | 0.9435091 | 0.8911144 | 0.6492016 | 0.7767964 | 1.1874931 |
| Phase-1 RCT-43 | 1.0045627 | 0.974783 | 1.0760778 | 1.0563263 | 0.7797094 | 0.7579429 |
| Tissue plasminogen activator | 0.8059819 | 0.9157869 | 0.9267975 | 1.0972991 | 0.8456018 | 1.0019314 |
| Ubiquitin conjugating enzyme (RAD 6 | 0.983925 | 0.9148318 | 1.06924 | 1.156948 | 1.0862191 | 0.9426346 |
| homologue) | | | | | | |
| Phase-1 RCT-173 | 1.3243454 | 0.8927029 | 0.8998468 | 1.1152853 | 1.0487052 | 0.9191367 |
| Ornithine decarboxylase | 0.8415299 | 1.028521 | 0.9666587 | 1.393947 | 1.6039736 | 0.9775671 |
| Zinc finger protein | 1.0678099 | 0.9188789 | 0.8720024 | 0.9309664 | 0.9106594 | 0.854264 |
| CDK108 | 1.0406368 | 0.9286849 | 0.6603304 | 0.7541694 | 1.072179 | 1.0037484 |
| Phase-1 RCT-117 | 1.4141141 | 0.6826147 | 1.0567884 | 1.0112221 | 1.0161958 | 1.5005915 |
| {Phase-1 RCT 98} | 0.795604 | 0.883294 | 0.9031604 | 0.750903 | 1.0174438 | 1.0648377 |
| Aquaporin-3 (AQP3) | 0.9976315 | 1.0270131 | 1.0010186 | 1.1204846 | 0.9541577 | 1.0749208 |
| Cholesterol esterase | 0.9804129 | 0.62838 | 0.6106226 | 0.2963703 | 0.6392945 | 1.0317659 |
| Ribosomal protein S17 | 1.0321016 | 1.0209662 | 1.3698387 | 1.7063991 | 0.8382872 | 0.9806467 |
| Poly(ADP-ribose) polymerase | 0.9327309 | 1.0987103 | 1.0924722 | 1.1368742 | 1.3425574 | 0.9384226 |
| Annexin V | 0.9704871 | 1.213216 | 1.3544004 | 1.0370871 | 1.3261613 | 1.0417982 |
| Phase-1 RCT-33 | 1.0777398 | 0.9030384 | 0.7818755 | 0.2524734 | 0.913493 | 1.321114 |
| Phase-1 RCT-61 | 1.0409776 | 1.14797 | 1.105878 | 1.1887727 | 0.9719888 | 0.9673656 |
| Retinol dehydrogenase type III | 0.9790127 | 1.0659686 | 1.1881565 | 0.3758506 | 0.5197056 | 1.3457546 |
| ATPase inhibitor (rat mitochondrial IF1 protein) | 1.0671028 | 0.9210184 | 0.9006414 | 0.3211214 | 0.3570914 | 1.1001531 |
| Thymidylate synthase | 1.0679888 | 0.9229166 | 1.198154 | 1.2031559 | 1.473826 | 0.9328892 |
| Interleukin-18 | 1.0661435 | 1.1950697 | 1.7243186 | 1.2496434 | 1.2243454 | 0.7853918 |
| Lecithin: cholesterol acyltransferase | 1.43006 | 0.9889379 | 1.4320257 | 0.7178342 | 0.7729059 | 1.1669066 |
| Contrapsin-like protease inhibitor (CPi-21) | 0.6427649 | 0.5335479 | 0.5804208 | 0.153969 | 0.6409084 | 1.083728 |
| Proliferating cell nuclear antigen gene | 0.8905549 | 0.9617404 | 1.3109872 | 1.1931351 | 1.8337828 | 0.9251331 |
| Phase-1 RCT-230 | 1.2258834 | 1.0276912 | 0.6108781 | 1.4854664 | 1.1695052 | 0.7341152 |
| Cytochrome P450 2D18 | 1.203407 | 1.0045012 | 1.3379599 | 0.5033404 | 0.8545001 | 1.1295297 |
| Phase-1 RCT-48 | 0.6909185 | 0.745936 | 0.6749886 | 0.5688186 | 1.085952 | 1.3497325 |
| Phase-1 RCT-292 | 0.9924922 | 1.0595169 | 1.0147758 | 1.0631226 | 0.9976942 | 0.986367 |
| Arginosuccinate synthetase 1 | 0.8433512 | 1.1891431 | 0.6602105 | 0.3391129 | 0.8418999 | 1.0692116 |
| C-reactive protein | 0.8192313 | 0.9555904 | 1.0991669 | 0.7286122 | 0.7938546 | 1.2653943 |

| | NAL | NAL | NAL | NAL | NAL | PBARB | PBARB |
|---|---|---|---|---|---|---|---|
| Compound-Dose | 45 | 45 | 180 | 180 | 180 | 20 | 20 |
| Animal Number | 2648 | 2649 | 2657 | 2658 | 2659 | 2627 | 2628 |
| Liver Toxicity Necrosis Classification | no | no | no | no | no | no | no |
| Gene Name | | | | | | | |
| Osteoactivin | 0.9147662 | 0.9891366 | 0.9489654 | 1.0668236 | 1.0490656 | 1.1000005 | 1.0104872 |
| Stathmin | 0.8712027 | 0.9386342 | 1.0976484 | 0.9299006 | 0.946978 | 0.8712996 | 0.8240463 |
| Calpactin I heavy chain | 0.9976999 | 1.0375831 | 1.0534219 | 1.1231315 | 0.971337 | 0.9773347 | 1.1016562 |
| Phase-1 RCT-179 | 1.0535889 | 1.114375 | 0.9217617 | 0.8967619 | 1.0041624 | 0.9389424 | 0.9013792 |
| Phase-1 RCT-107 | 0.9485946 | 0.9083006 | 0.9442056 | 0.8243185 | 1.1768436 | 1.5979345 | 1.0028946 |
| Heme oxygenase | 0.9406061 | 0.9229921 | 0.8289228 | 1.0682218 | 0.9523256 | 0.854371 | 0.9872379 |
| Collagen type II | 0.7436398 | 0.7614648 | 0.8507482 | 0.8196262 | 0.7320131 | 0.8930218 | 0.7775022 |
| Phase-1 RCT-81 | 1.0859071 | 1.2039735 | 1.218669 | 1.2427287 | 1.2024243 | 1.3928008 | 1.4242733 |
| Glycine methyltransferase | 1.0164379 | 0.9361526 | 0.905613 | 0.7576993 | 1.4146914 | 2.4705284 | 1.1170149 |
| Cystatin C | 0.8682982 | 0.9291872 | 1.1216093 | 1.1078054 | 1.0402185 | 0.9352144 | 0.98972 |
| Melanoma-associated antigen ME491 | 0.9815267 | 1.3411556 | 0.9306477 | 1.0324688 | 0.9041084 | 0.8442448 | 0.9756926 |
| Phase-1 RCT-146 | 0.8264166 | 0.8519853 | 1.0970615 | 0.8305411 | 0.8020375 | 0.9175733 | 0.7133088 |
| Gamma-actin, cytoplasmic | 0.7677951 | 0.7669878 | 0.7173405 | 1.1920035 | 1.3997043 | 0.9244225 | 0.9858874 |
| Adenine nucleotide translocator 1 | 1.0408248 | 0.8727914 | 0.8480772 | 0.8542812 | 0.7029305 | 0.7183636 | 0.8490511 |
| Thymosin beta-10 | 1.008284 | 0.9414385 | 1.0216744 | 1.1006081 | 0.8206765 | 0.8981913 | 0.9410115 |
| Insulin-like growth factor I, exon 6 | 1.051881 | 0.9988926 | 0.9863029 | 0.8865702 | 1.4880875 | 1.0043168 | 1.5858485 |
| Phase-1 RCT-78 | 0.9361455 | 1.1376109 | 1.0002902 | 1.0339024 | 1.393094 | 1.4763999 | 1.6079495 |
| Alpha-2-microglobulin | 1.2563403 | 1.1587491 | 0.6779439 | 0.8013968 | 1.893734 | 1.373557 | 1.1115515 |
| High affinity IgE receptor gamma chain | 1.0750806 | 1.0251201 | 0.9633488 | 1.0869501 | 1.0565835 | 0.9597281 | 1.0188864 |
| (FcERIgamma) | | | | | | | |
| 60S ribosomal protein L6 | 1.1267233 | 1.1853625 | 1.1682616 | 1.2099817 | 0.9361768 | 1.0749744 | 1.0324316 |
| IgE binding protein | 0.9546779 | 0.8858375 | 0.8255485 | 0.8845342 | 0.9028289 | 0.9654349 | 1.0450615 |
| Phase-1 RCT-149 | 0.9200097 | 1.056713 | 1.1715338 | 1.1082515 | 0.9357019 | 0.9872924 | 0.8465427 |
| Cytochrome P450 2C11 | 0.8299488 | 0.8704967 | 1.0194743 | 0.9457214 | 0.8573985 | 0.9957351 | 0.8060056 |
| Uncoupling protein 2 | 0.9193886 | 0.8503677 | 0.96035 | 1.1411486 | 0.8583807 | 0.8432796 | 0.9157972 |
| Cofilin | 1.0720181 | 1.0992175 | 1.2554569 | 1.2933493 | 1.1867094 | 1.3134465 | 1.3372964 |
| Alpha-tubulin | 1.4371778 | 1.2269413 | 1.2376714 | 1.2658547 | 0.8089383 | 0.7904624 | 0.7937493 |
| Stearyl-CoA desaturase, liver | 0.8208221 | 0.9310387 | 0.981368 | 1.5956132 | 0.9880405 | 0.7579071 | 0.8322318 |
| Glutathione S-transferase theta-1 | 1.0801333 | 1.1908382 | 1.1447628 | 1.3846014 | 1.1876476 | 1.2942436 | 1.1913857 |
| alpha-1,2-fucosyltrans-ferase | 0.8058795 | 0.8373382 | 0.7062721 | 0.7746964 | 1.0085449 | 0.7964341 | 0.8786712 |
| Phase-1 RCT-138 | 1.0051467 | 0.9958098 | 1.1222726 | 1.0250318 | 1.2239394 | 1.2918928 | 1.2019229 |
| Phase-1 RCT-295 | 1.0436698 | 1.1315644 | 0.9435917 | 1.1651349 | 1.2267755 | 1.0756252 | 1.1605604 |
| Phase-1 RCT-164 | 1.3414037 | 1.2770928 | 1.2028285 | 1.1952517 | 0.8994938 | 0.890294 | 0.8844867 |
| Alpha-2-macroglobulin, sequence 2 | 0.7656277 | 0.8290985 | 1.0234447 | 0.9669398 | 0.9357274 | 1.0523249 | 0.9966801 |
| Biliverdin reductase | 1.0863231 | 1.0942054 | 1.1467558 | 1.2478743 | 0.9228338 | 0.8491895 | 0.870301 |
| Phase-1 RCT-121 | 0.8171266 | 0.8495047 | 0.8637909 | 0.760667 | 0.7964258 | 0.7060736 | 0.7143425 |
| Dynamin-1 (D100) | 0.9533131 | 0.9249783 | 0.8920444 | 1.0732712 | 0.9450948 | 1.1911267 | 1.0122385 |
| Phase-1 RCT-192 | 0.9556371 | 0.8917921 | 0.9880742 | 0.9698997 | 0.792768 | 0.9287247 | 0.7198688 |
| Fatty acid synthase | 0.7844151 | 0.9711194 | 0.9726406 | 2.1169987 | 0.63064 | 0.5953478 | 0.4457553 |
| Phase-1 RCT-211 | 0.8762057 | 0.9428728 | 0.8645449 | 0.8745469 | 0.9468275 | 0.8896761 | 0.8192912 |
| Ribosomal protein S9 | 1.2888178 | 1.4568515 | 1.410056 | 1.0602819 | 0.7508149 | 0.6785888 | 0.3082534 |
| NADP-dependent isocitrate dehydrogenase, | 1.2307334 | 1.1665051 | 1.095657 | 1.3802017 | 1.0833308 | 1.0192965 | 0.9883815 |
| cytosolic | | | | | | | |
| Phase-1 RCT-57 | 1.0350158 | 1.060021 | 1.0811781 | 1.142453 | 0.948064 | 1.1706275 | 0.9954169 |
| Phase-1 RCT-36 | 1.0659205 | 1.1130673 | 1.0762144 | 1.3176169 | 0.932559 | 0.9524562 | 1.0023878 |
| Apolipoprotein All | 0.6310686 | 0.6620002 | 0.6888653 | 0.6023305 | 1.1117367 | 1.3497356 | 1.1971053 |
| Phase-1 RCT-68 | 1.0123672 | 1.0937538 | 0.9742795 | 1.037109 | 0.9930121 | 0.8502379 | 0.916113 |
| Multidrug resistant protein-2 | 1.1612363 | 0.9379889 | 1.1518694 | 1.3727981 | 0.9021665 | 0.8302945 | 0.8297809 |
| Phase-1 RCT-39 | 0.8398247 | 0.7429932 | 0.7974219 | 0.8232224 | 0.7000519 | 0.761114 | 0.8336013 |
| NIPK | 1.0254208 | 0.9370484 | 0.958658 | 0.8902682 | 1.0032623 | 0.8130045 | 1.068172 |
| F1-ATPase beta subunit | 1.1431013 | 1.2680589 | 1.2522893 | 1.26593 | 1.3564028 | 1.1587538 | 1.3672363 |
| Elongation factor-1 alpha | 1.0308582 | 1.0532014 | 0.9986461 | 1.0869707 | 1.2577198 | 1.1261634 | 1.1566188 |
| Phase-1 RCT-24 | 0.9308486 | 0.8957918 | 0.9241459 | 0.8431504 | 0.7648016 | 0.851857 | 0.8135715 |
| Beta-tubulin, class I | 1.0585823 | 1.0105604 | 1.033891 | 1.1811792 | 1.0480233 | 0.7786593 | 0.928995 |
| 14-3-3 zeta | 1.1000648 | 1.090074 | 0.7545614 | 1.2013283 | 1.0731572 | 0.8514728 | 1.4176291 |
| Alpha-prothymosin | 1.0476112 | 1.1445866 | 1.0793945 | 1.3827416 | 1.4588745 | 1.6149164 | 1.9328578 |
| Phase-1 RCT-58 | 0.7691084 | 0.5414674 | 0.5267108 | 0.8934351 | 0.6144975 | 0.751259 | 0.854617 |
| Phase-1 RCT-290 | 1.029976 | 1.0831784 | 1.225132 | 1.4778811 | 2.0695183 | 2.647869 | 1.8948996 |
| Transitional endoplasmic reticulum ATPase | 1.4392047 | 1.4635763 | 1.3296083 | 1.4265857 | 0.9156389 | 0.9454832 | 0.8359669 |
| Beta-actin | 0.8977177 | 0.7450988 | 0.4068947 | 1.3621112 | 1.5519385 | 1.0467507 | 1.2063875 |
| Thioredoxin-1 (Trx1) | 0.9474517 | 0.9551458 | 0.8148626 | 0.8106747 | 1.0795963 | 1.1099467 | 1.0506935 |
| Phase-1 RCT-174 | 1.1909225 | 1.3138336 | 1.1230534 | 1.0650371 | 0.8883016 | 1.1048603 | 0.9722873 |
| Beta-actin, sequence 2 | 1.0407416 | 0.9617327 | 0.8996311 | 1.2185603 | 1.3021172 | 1.1109042 | 1.2332952 |
| Phase-1 RCT-109 | 0.9944954 | 0.9818392 | 0.9991325 | 0.9706016 | 0.9787259 | 0.8921556 | 0.928609 |
| Phase-1 RCT-154 | 0.9238402 | 1.0850054 | 1.0118535 | 1.1629112 | 0.9255705 | 0.896789 | 0.8652214 |
| Hypoxanthine-guanine | 1.2005963 | 1.173632 | 0.9414581 | 1.2987282 | 0.8667651 | 0.7932474 | 0.835735 |
| phosphoribosyltransferase | | | | | | | |
| Phase-1 RCT-213 | 1.0400016 | 0.9944027 | 0.9757901 | 1.0816636 | 0.9337286 | 0.9580717 | 1.0163246 |
| Phase-1 RCT-260 | 0.817779 | 0.7835026 | 0.826214 | 0.7369392 | 0.9009819 | 0.7721994 | 0.8437855 |
| Phase-1 RCT-12 | 0.9939669 | 0.9814422 | 1.0443068 | 1.0694181 | 0.9185681 | 0.8827791 | 0.8925366 |
| Phase-1 RCT-293 | 1.0022143 | 0.9962469 | 1.0828778 | 0.9594031 | 0.9753978 | 1.1142173 | 1.142178 |
| Ribosomal protein S8 | 1.0118034 | 1.0352688 | 1.0995412 | 1.0208149 | 1.0958484 | 1.1578766 | 1.1258156 |
| Pyruvate kinase, muscle | 1.1428627 | 1.0726794 | 1.0201123 | 1.0586416 | 0.9792394 | 1.0011768 | 1.0607005 |
| Nucleoside diphosphate kinase beta isoform | 0.7866095 | 0.8573965 | 0.9340763 | 0.867805 | 0.8120618 | 0.8641955 | 0.8450446 |
| p55CDC | 0.7250332 | 0.8082185 | 1.3501107 | 0.8625534 | 0.9495609 | 0.9698818 | 0.6880444 |
| Phase-1 RCT-156 | 1.0127658 | 1.0474192 | 0.9710062 | 0.9812501 | 0.898312 | 0.9220299 | 1.0800399 |
| Ribosomal protein L13A | 0.987346 | 1.0036466 | 0.8897415 | 0.9997685 | 0.822236 | 0.7668398 | 0.8828662 |
| Phase-1 RCT-258 | 0.9882755 | 1.0247308 | 1.0437845 | 1.0685399 | 0.9196098 | 0.9847405 | 1.0163355 |
| Insulin-like growth factor I | 1.0688092 | 1.2108288 | 1.0353371 | 0.8298581 | 1.3047029 | 1.1052954 | 1.3137918 |
| Cytochrome P450 3A1 | 1.3239988 | 1.3113935 | 0.9978398 | 0.8068293 | 1.0662622 | 1.1218323 | 0.858252 |
| {Ribosomal protein 16} | 1.018145 | 1.0005444 | 1.0300326 | 1.112784 | 1.1594827 | 1.1405401 | 1.1024618 |
| Organic cation transporter 3 | 1.0635039 | 1.1265732 | 1.1415148 | 1.1856589 | 0.8398094 | 1.0643888 | 1.0734493 |
| Calpain 2 | 0.9306198 | 0.9583113 | 1.0093716 | 1.0469586 | 0.9403505 | 1.058296 | 1.0259593 |
| Phase-1 RCT-102 | 0.7430327 | 1.1820596 | 0.9621376 | 0.8313277 | 0.9148487 | 0.9319918 | 0.9102434 |
| Equilbrative nitrobenzylthioinosine-sensitive | 1.005657 | 3.405299 | 1.0076522 | 0.8627828 | 1.0573655 | 1.0110977 | 0.957339 |
| nucleoside transporter | | | | | | | |
| Phase-1 RCT-227 | 0.8183033 | 1.021913 | 0.8564208 | 0.895339 | 1.7193093 | 2.1500123 | 2.136068 |
| N-hydroxy-2-acetylaminofluorene sulfotransferase (ST1C1) | 1.137288 | 1.1289864 | 0.9027274 | 0.9753758 | 1.1820946 | 0.9856857 | 1.1377486 |
| Cathepsin S | 1.361804 | 1.0594404 | 0.8828356 | 1.1586075 | 1.1907812 | 0.9784386 | 1.1090113 |
| Apolipoprotein CIII | 1.4012952 | 1.1493348 | 1.0963252 | 1.2348676 | 1.1810548 | 1.3965524 | 1.2922933 |
| Cytochrome P450 2A3 | 1.4892892 | 1.232114 | 1.5128523 | 2.0122929 | 1.1789479 | 1.2803565 | 1.2493452 |
| Cytochrome P450 2C23 | 1.017396 | 1.1359179 | 1.037053 | 1.1369281 | 1.343562 | 1.488157 | 1.4566234 |
| Apolipoprotein C1 | 1.093269 | 1.0397438 | 1.2117387 | 1.2138997 | 1.4307657 | 1.4583184 | 1.4304458 |
| Betaine homocysteine methyltransferase (BHMT) | 1.0031781 | 1.0838253 | 1.1963247 | 1.5138712 | 2.465419 | 3.905292 | 2.6158795 |
| Paraoxonase 1 | 1.0992813 | 1.0793746 | 1.1295761 | 1.1853142 | 1.2894782 | 1.4267334 | 1.3624862 |
| Phase-1 RCT-207 | 1.1295588 | 0.9133348 | 0.9513911 | 1.1010283 | 0.9267963 | 0.8706352 | 0.8306841 |
| Cathepsin B | 1.1422342 | 1.103397 | 0.9264547 | 0.9620208 | 1.3059382 | 1.1585654 | 1.3070632 |
| Phase-1 RCT-144 | 1.0622104 | 1.1060777 | 1.0863566 | 1.1475734 | 0.808529 | 0.8662474 | 0.8284239 |
| Alpha-1 microglobulin/bikunin precursor (Ambp) | 1.1112113 | 1.2111702 | 1.2383049 | 1.2688946 | 1.3904756 | 1.4859713 | 1.4516711 |
| Cyclin D1 | 1.0504048 | 1.1382887 | 1.2570727 | 1.164803 | 0.7293428 | 0.7326466 | 0.7668667 |
| Presenilin-1 | 1.0369233 | 1.2396995 | 1.1041774 | 0.7235389 | 1.6484641 | 1.9073311 | 1.9917859 |
| Protein tyrosine phosphatase, receptor type, D | 0.9883894 | 0.982193 | 0.9555584 | 1.2110115 | 2.15285 | 1.4726137 | 1.9470203 |
| Multidrug resistant protein-1 | 1.283138 | 1.0949279 | 1.2685359 | 0.9871832 | 0.7808962 | 0.8187904 | 0.9234994 |
| Cathepsin L, sequence 2 | 1.3409743 | 1.0505468 | 1.1865433 | 1.0333443 | 0.7841266 | 0.8503167 | 0.862914 |
| JNK1 stress activated protein kinase | 1.2867584 | 0.9343085 | 1.2117618 | 1.2807126 | 1.8793037 | 1.9044508 | 1.3127513 |
| Phase-1 RCT-43 | 0.8349865 | 0.8926138 | 0.8830552 | 0.864736 | 0.7819737 | 0.8186833 | 0.7400571 |
| Tissue plasminogen activator | 0.9364673 | 0.5375308 | 1.01193 | 0.9001902 | 0.8899391 | 0.9154534 | 1.0578601 |
| Ubiquitin conjugating enzyme (RAD 6 | 1.2392 | 1.4143056 | 1.3813927 | 1.1535285 | 0.8262869 | 0.7435762 | 0.5849583 |
| homologue) | | | | | | | |
| Phase-1 RCT-173 | 1.0867364 | 1.2446572 | 1.2551767 | 1.076961 | 0.794446 | 0.9793871 | 0.8890109 |
| Ornithine decarboxylase | 1.0766292 | 1.0435845 | 0.8351467 | 1.0805436 | 1.1510037 | 1.0711745 | 1.0331295 |
| Zinc finger protein | 0.9433262 | 1.0575773 | 0.9849559 | 1.1685404 | 0.9003331 | 0.8968357 | 1.0073584 |
| CDK108 | 0.7813435 | 1.2322638 | 1.1685582 | 0.9207891 | 0.8904851 | 1.2349443 | 1.1015999 |
| Phase-1 RCT-117 | 1.5140814 | 1.0528492 | 1.5186309 | 1.079518 | 1.637873 | 1.8335812 | 1.7035277 |
| {Phase-1 RCT 98} | 1.1183951 | 1.050586 | 1.1537381 | 1.0156674 | 1.0995042 | 1.2098362 | 1.1651249 |
| Aquaporin-3 (AQP3) | 0.9611254 | 1.0465581 | 1.0107428 | 0.9872055 | 0.8189727 | 0.8742755 | 0.878429 |
| Cholesterol esterase | 1.3837117 | 1.5046531 | 1.3082937 | 0.896692 | 1.1219682 | 1.4348034 | 1.1860919 |
| Ribosomal protein S17 | 0.975534 | 1.022062 | 1.0374516 | 0.9893299 | 0.9635388 | 1.1225786 | 0.9085316 |
| Poly(ADP-ribose) polymerase | 1.1346192 | 1.2707748 | 1.1760064 | 1.2670895 | 0.9277544 | 0.8794912 | 0.8786052 |
| Annexin V | 0.9294087 | 1.2255098 | 0.9173401 | 0.9495528 | 0.9861434 | 1.1607822 | 0.8174758 |
| Phase-1 RCT-33 | 1.2540448 | 1.3018064 | 1.0874577 | 1.701169 | 0.9873421 | 0.978245 | 1.0170299 |
| Phase-1 RCT-61 | 0.8197731 | 0.7657454 | 0.828942 | 0.9959782 | 0.7897525 | 0.7902984 | 0.7810313 |
| Retinol dehydrogenase type III | 1.3563033 | 1.3682832 | 1.2232559 | 1.4808483 | 1.0682825 | 1.1292253 | 1.08811 |
| ATPase inhibitor (rat mitochondrial IF1 protein) | 1.0795641 | 0.992469 | 1.0183364 | 0.9484351 | 0.9219881 | 1 | 0.8513641 |
| Thymidylate synthase | 0.9525469 | 0.8695241 | 1.0623364 | 0.8069348 | 1 | 1.1909848 | 0.8599663 |
| Interleukin-18 | 0.7450122 | 0.8061101 | 0.9233593 | 0.8282869 | 0.8329167 | 0.9006895 | 0.8231207 |
| Lecithin: cholesterol acyltransferase | 1.2380159 | 1.1217691 | 1.0310658 | 1.2385458 | 1.357016 | 1.3218193 | 1.3407273 |
| Contrapsin-like protease inhibitor (CPi-21) | 0.9146368 | 0.9100239 | 0.8827826 | 0.9011198 | 1.3611203 | 1.269535 | 1.9963586 |
| Proliferating cell nuclear antigen gene | 0.9863851 | 0.966215 | 1.0750554 | 0.9811896 | 0.8534624 | 0.7788727 | 1.1742791 |
| Phase-1 RCT-230 | 0.7914896 | 0.7898556 | 0.9883319 | 0.7545182 | 0.732672 | 0.8211702 | 0.7070233 |
| Cytochrome P450 2D18 | 1.1131692 | 1.1961411 | 1.0298856 | 1.0851332 | 1.1026638 | 1.3706409 | 1.1956983 |
| Phase-1 RCT-48 | 0.9983832 | 1.375162 | 0.7504953 | 0.9469585 | 1.8123668 | 1.6284145 | 1.9732299 |
| Phase-1 RCT-292 | 0.9728045 | 0.9648497 | 1.0615056 | 0.9346614 | 1.15737 | 1.2172015 | 1.1697378 |
| Arginosuccinate synthetase 1 | 1.27048 | 0.9942617 | 1.221726 | 1.3348238 | 1.7383666 | 1.6192597 | 1.3627461 |
| C-reactive protein | 1.0740874 | 1.1203426 | 0.9861776 | 1.2944843 | 1.762741 | 1.6754831 | 1.9086064 |

| | PBARB | PBARB | PBARB | PBARB | PEG | PEG |
|---|---|---|---|---|---|---|
| Compound-Dose | 20 | 80 | 80 | 80 | 5000 | 5000 |
| Animal Number | 2629 | 2637 | 2638 | 2639 | 147 | 148 |
| Liver Toxicity Necrosis Classification | no | no | no | no | no | no |
| Gene Name | | | | | | |
| Osteoactivin | 1.0681022 | 1.020692 | 0.9911286 | 1.0095752 | 1.0243467 | 1.1038651 |
| Stathmin | 0.9493735 | 0.9496202 | 0.9628286 | 0.9997175 | 1.0348947 | 0.9930269 |
| Calpactin I heavy chain | 0.9987183 | 1.0200584 | 1.00913 | 0.9321313 | 1.0636147 | 0.9980707 |
| Phase-1 RCT-179 | 1.0375403 | 0.9665334 | 1.0143441 | 1.0893667 | 1.1870441 | 1.1313825 |
| Phase-1 RCT-107 | 1.2903292 | 0.9318629 | 0.8833773 | 1.1227789 | 1.0361755 | 0.9377657 |
| Heme oxygenase | 0.8836949 | 0.8570268 | 0.8659331 | 0.8625652 | 0.8902164 | 1.4914275 |
| Collagen type II | 0.6884517 | 0.8482933 | 0.8741081 | 0.7687087 | 1.463999 | 0.9234915 |
| Phase-1 RCT-81 | 1.2844416 | 1.2126713 | 1.1101803 | 1.1521755 | 1.0371921 | 0.9939237 |
| Glycine methyltransferase | 1.7575016 | 0.9720452 | 0.897971 | 1.4945792 | 1.5294602 | 0.8367874 |
| Cystatin C | 0.7354955 | 1.252816 | 1.0925428 | 1.2398707 | 1.2353616 | 0.9857466 |
| Melanoma-associated antigen ME491 | 0.8921669 | 0.8623412 | 0.8815501 | 0.8455257 | 1.2210563 | 1.2444118 |
| Phase-1 RCT-146 | 0.8049082 | 0.8354396 | 0.8801082 | 0.7911438 | 1.0328479 | 0.9935144 |
| Gamma-actin, cytoplasmic | 1.5039393 | 0.9130033 | 0.8772457 | 0.7291561 | 0.7420574 | 1.0207636 |
| Adenine nucleotide translocator 1 | 0.6951975 | 0.833414 | 1.0005333 | 0.8819355 | 1.1447226 | 1.1191316 |
| Thymosin beta-10 | 0.7858071 | 0.8749503 | 0.8513818 | 0.8893719 | 1.4785045 | 1.0198871 |
| Insulin-like growth factor I, exon 6 | 1.5650039 | 1.1487107 | 1.0635723 | 1.2018175 | 0.694047 | 0.6636308 |
| Phase-1 RCT-78 | 1.5177312 | 1.393667 | 1.2090902 | 0.9923621 | 0.9693164 | 0.9540514 |
| Alpha-2-microglobulin | 2.1022305 | 1.4788824 | 1.0920517 | 1.4406638 | 1.210603 | 0.4648515 |
| High affinity IgE receptor gamma chain | 1.1409408 | 0.9577985 | 1.0208466 | 0.8726915 | 1.1324425 | 1.1602678 |
| (FcERIgamma) | | | | | | |
| 60S ribosomal protein L6 | 0.8798761 | 1.0515441 | 1.0748861 | 1.0293868 | 1.1392695 | 1.1411678 |
| IgE binding protein | 0.951803 | 1.0220971 | 1.016634 | 0.7316075 | 1.0138836 | 1.1052861 |
| Phase-1 RCT-149 | 0.9493878 | 1.0070698 | 0.9026027 | 0.9304614 | 0.7319083 | 0.7758704 |
| Cytochrome P450 2C11 | 0.8369874 | 0.9706708 | 1.0863271 | 0.8129715 | 1.1524162 | 0.7082815 |
| Uncoupling protein 2 | 0.8363673 | 0.8900666 | 0.8258673 | 0.8135876 | 1.1343237 | 1.0152855 |
| Cofilin | 1.2154084 | 1.2362891 | 1.314074 | 1.28102061 | 0.9734651 | 1.0900774 |
| Alpha-tubulin | 0.785783 | 0.9409534 | 0.9505962 | 0.8416742 | 0.9287966 | 1.1379485 |
| Stearyl-CoA desaturase, liver | 1.0935715 | 0.470602 | 1.1015036 | 0.8207721 | 1.3361676 | 1.2032986 |
| Glutathione S-transferase theta-1 | 1.1625053 | 1.3391485 | 1.0719215 | 0.8566172 | 0.9160208 | 1.3319447 |
| alpha-1,2-fucosyltrans-ferase | 1.063562 | 0.9263592 | 0.8941866 | 0.8777449 | 0.9738569 | 1.1146945 |
| Phase-1 RCT-138 | 1.354122 | 0.9423161 | 1.0727934 | 0.9583547 | 0.9695967 | 0.8740953 |
| Phase-1 RCT-295 | 1.2807249 | 1.0950618 | 1.0731391 | 1.1032711 | 1.0539957 | 1.2049714 |
| Phase-1 RCT-164 | 0.8967226 | 0.9066482 | 0.9806896 | 1.0709443 | 1.1009766 | 1.0661381 |
| Alpha-2-macroglobulin, sequence 2 | 0.9675844 | 0.8991543 | 0.9863888 | 1.0224123 | 1.144344 | 1.102659 |
| Biliverdin reductase | 0.8434774 | 0.7549841 | 0.8072573 | 0.7829774 | 0.7883885 | 0.9880469 |
| Phase-1 RCT-121 | 0.7711266 | 0.7931917 | 0.8704031 | 0.7228972 | 1.042279 | 1.0291678 |
| Dynamin-1 (D100) | 0.9416267 | 1.1046423 | 1.2052202 | 1.1579372 | 1.1558326 | 1.123796 |
| Phase-1 RCT-192 | 0.7512921 | 0.822525 | 1.0265126 | 0.8808383 | 1.2404513 | 1.2106684 |
| Fatty acid synthase | 0.5827688 | 0.4185709 | 0.578737 | 0.4227541 | 1.4525114 | 1.9735835 |
| Phase-1 RCT-211 | 1.0118366 | 0.8789988 | 0.9243776 | 0.9169517 | 1.0130594 | 0.9940469 |
| Ribosomal protein S9 | 0.7762632 | 0.9011875 | 0.9506678 | 0.8976668 | 1.2393398 | 1.1423813 |
| NADP-dependent isocitrate dehydrogenase, | 1.0729548 | 1.1179047 | 1.1372709 | 1.3077935 | 1.2367214 | 1.4857517 |
| cytosolic | | | | | | |
| Phase-1 RCT-57 | 0.9737081 | 1.0214071 | 1.1396002 | 1.0132948 | 0.9227815 | 0.8593329 |
| Phase-1 RCT-36 | 1.016066 | 0.8553294 | 1.0026053 | 0.9233998 | 0.904835 | 1.2047985 |
| Apolipoprotein All | 1.0809634 | 0.7587417 | 0.8718275 | 0.6704185 | 1.1926595 | 1.4729168 |
| Phase-1 RCT-68 | 1.032685 | 0.9770305 | 0.9844192 | 0.9337971 | 0.9155279 | 0.9601467 |
| Multidrug resistant protein-2 | 0.8012325 | 0.8182293 | 0.8610359 | 1.0549647 | 0.8019903 | 0.6527317 |
| Phase-1 RCT-39 | 0.6517676 | 0.6979146 | 0.7892513 | 0.765115 | 0.9570239 | 0.9397483 |
| NIPK | 1.0392846 | 0.8380644 | 0.9356745 | 0.9014136 | 0.8908796 | 0.9875944 |
| F1-ATPase beta subunit | 1.4205534 | 1.1740105 | 1.1810247 | 1.2794187 | 1.1916652 | 1.0058454 |
| Elongation factor-1 alpha | 1.2334007 | 1.1535013 | 1.3147627 | 1.3048073 | 1.0857375 | 1.0625197 |
| Phase-1 RCT-24 | 0.7897209 | 0.8274485 | 0.7796192 | 0.6784555 | 0.8689429 | 1.1844808 |
| Beta-tubulin, class I | 1.0754402 | 0.8870018 | 0.8323472 | 0.6674054 | 0.7874237 | 1.0494907 |
| 14-3-3 zeta | 1.0420884 | 0.9153783 | 1.0054821 | 0.8743569 | 0.8139162 | 0.8192905 |
| Alpha-prothymosin | 1.5526446 | 1.3135582 | 1.425532 | 1.3598168 | 1.0831113 | 0.8723897 |
| Phase-1 RCT-58 | 0.6227589 | 0.7842403 | 0.7150964 | 0.6937547 | 0.9140953 | 1.1609364 |
| Phase-1 RCT-290 | 2.3765686 | 1.2950222 | 1.031606 | 1.8142194 | 1.1854224 | 0.7086721 |
| Transitional endoplasmic reticulum ATPase | 0.8619606 | 1.0466201 | 0.9190549 | 1.0042983 | 0.9596976 | 0.9151325 |
| Beta-actin | 1.5262188 | 0.9682944 | 0.9747731 | 0.877177 | 0.4915266 | 0.5224849 |
| Thioredoxin-1 (Trx1) | 1.2169228 | 0.9735302 | 1.0100622 | 0.9673498 | 1.0968964 | 1.061946 |
| Phase-1 RCT-174 | 0.9409074 | 1.1072148 | 1.009454 | 0.9101424 | 1.1227518 | 0.993932 |
| Beta-actin, sequence 2 | 1.4097862 | 1.0162219 | 0.9757723 | 0.9013609 | 1.0122339 | 0.981077 |
| Phase-1 RCT-109 | 1.0026973 | 0.8244018 | 0.9266145 | 0.7959788 | 1.1872802 | 1.1042807 |
| Phase-1 RCT-154 | 0.8990485 | 1.0172708 | 0.92075 | 0.9386597 | 1.0273411 | 1.0960258 |
| Hypoxanthine-guanine | 0.8549901 | 0.9122561 | 0.917501 | 0.8474942 | 0.8430402 | 0.9179947 |
| phosphoribosyltransferase | | | | | | |
| Phase-1 RCT-213 | 0.9067254 | 1.0142208 | 0.9103023 | 0.8689073 | 0.9609211 | 0.9838675 |
| Phase-1 RCT-260 | 0.8335188 | 0.6711952 | 0.812216 | 0.7736219 | 1.025524 | 0.9023159 |
| Phase-1 RCT-12 | 0.9470108 | 0.8184652 | 0.8972786 | 0.7743334 | 0.781499 | 0.9499077 |
| Phase-1 RCT-293 | 0.9959736 | 1.506845 | 1.0875044 | 1.1438954 | 1.1349994 | 1.077701 |
| Ribosomal protein S8 | 1.1589929 | 1.086008 | 1.1098642 | 1.0618838 | 1.2314607 | 1.148644 |
| Pyruvate kinase, muscle | 0.9975391 | 1.0104824 | 0.8898286 | 0.8340716 | 0.9410275 | 1.020891 |
| Nucleoside diphosphate kinase beta isoform | 0.8300374 | 0.9945509 | 1.0163077 | 0.8326426 | 0.9596526 | 1.0179305 |
| p55CDC | 0.9160835 | 1.1891092 | 1.0034987 | 0.799623 | 0.9824246 | 1.0494255 |
| Phase-1 RCT-156 | 0.8726671 | 0.8781474 | 1.2154782 | 0.8523893 | 0.6510842 | 0.6845373 |
| Ribosomal protein L13A | 0.7569482 | 0.8542667 | 0.8520774 | 0.794499 | 1.2656443 | 1.1776992 |
| Phase-1 RCT-258 | 0.9287484 | 1.0066423 | 1.0217731 | 0.9198902 | 1.0376508 | 1.0092086 |
| Insulin-like growth factor I | 1.4812555 | 1.1055781 | 1.0158107 | 1.2005968 | 0.7882857 | 0.7399306 |
| Cytochrome P450 3A1 | 1.1425034 | 2.3639562 | 1.8077859 | 2.0451283 | 1.348377 | 0.5963042 |
| {Ribosomal protein 16} | 1.2132626 | 1.0049261 | 1.1147923 | 1.0351973 | 1.0381678 | 1.0454322 |
| Organic cation transporter 3 | 0.8608773 | 1.0245893 | 1.0482843 | 0.9841865 | 1.0932629 | 1.1319125 |
| Calpain 2 | 0.9983848 | 0.9275092 | 0.9533305 | 0.9140133 | 1.0077493 | 0.9996519 |
| Phase-1 RCT-102 | 0.8988829 | 1.0345948 | 0.9325219 | 0.9670129 | 0.8808992 | 0.5893461 |
| Equilbrative nitrobenzylthioinosine-sensitive | 1.0457208 | 0.9205081 | 1.1255362 | 1.014275 | 1.174826 | 1.3316534 |
| nucleoside transporter | | | | | | |
| Phase-1 RCT-227 | 2.149468 | 1.7654392 | 1.868248 | 1.6628165 | 1.4214001 | 1.0657364 |
| N-hydroxy-2-acetylaminofluorene sulfotransferase (ST1C1) | 1.338185 | 0.9257401 | 1.2707722 | 1.3762817 | 0.9470569 | 0.6648127 |
| Cathepsin S | 1.1796762 | 1.0986004 | 1.0154128 | 1.1252106 | 1.1197613 | 1.2823292 |
| Apolipoprotein CIII | 1.1644843 | 1.1220003 | 1.0884417 | 1.2403752 | 1.2434987 | 1.075884 |
| Cytochrome P450 2A3 | 1.2133112 | 1.034832 | 0.8735153 | 1.1027521 | 1.5482737 | 1.4141785 |
| Cytochrome P450 2C23 | 1.3457034 | 1.2638164 | 1.351868 | 1.5591495 | 1.5492147 | 1.1754739 |
| Apolipoprotein C1 | 1.6201338 | 1.1559 | 1.2882545 | 1.1101732 | 1.4328041 | 1.1168059 |
| Betaine homocysteine methyltransferase (BHMT) | 3.3637373 | 1.4573135 | 1.1002183 | 2.431861 | 1.3200847 | 0.5840401 |
| Paraoxonase 1 | 1.394882 | 1.2847519 | 1.4326378 | 1.8130792 | 1.2259889 | 1.2462178 |
| Phase-1 RCT-207 | 0.9284737 | 0.9647198 | 0.9595952 | 0.9372601 | 0.9713334 | 0.9585154 |
| Cathepsin B | 1.2807446 | 1.3415164 | 1.0504866 | 1.2152315 | 1.1723552 | 0.9510422 |
| Phase-1 RCT-144 | 0.7314596 | 0.8175564 | 0.85144 | 0.8112554 | 0.8997876 | 0.9947886 |
| Alpha-1 microglobulin/bikunin precursor (Ambp) | 1.3774635 | 1.2516892 | 1.2349813 | 1.3297697 | 1.1595097 | 0.9529617 |
| Cyclin D1 | 0.6669847 | 0.8144977 | 0.7898943 | 0.7456075 | 1.0734183 | 0.6931113 |
| Presenilin-1 | 1.8179574 | 1.3948231 | 1.2937711 | 1.880646 | 1.0305552 | 0.8381836 |
| Protein tyrosine phosphatase, receptor type, D | 2.4399836 | 1.8800654 | 1.6058899 | 1.7617741 | 0.9325175 | 0.8165339 |
| Multidrug resistant protein-1 | 0.7047332 | 0.8350781 | 0.9202583 | 1.0028453 | 0.8104734 | 0.6503245 |
| Cathepsin L, sequence 2 | 0.8355236 | 0.957256 | 1.0639867 | 1.3069149 | 1.1757673 | 1.1614711 |
| JNK1 stress activated protein kinase | 2.0389585 | 1.423925 | 1.3374652 | 1.8151397 | 1.3268613 | 1.0379546 |
| Phase-1 RCT-43 | 0.801907 | 0.941738 | 0.911274 | 0.8377684 | 0.7581056 | 0.7601363 |
| Tissue plasminogen activator | 0.9198898 | 0.9471833 | 1.1046176 | 1.099658 | 0.9535774 | 0.9495595 |
| Ubiquitin conjugating enzyme (RAD 6 | 0.8265197 | 0.8550425 | 0.92207 | 0.9411432 | 1.223601 | 1.0746208 |
| homologue) | | | | | | |
| Phase-1 RCT-173 | 0.7762404 | 0.9996395 | 0.9918329 | 0.859489 | 1.0625894 | 0.9149621 |
| Ornithine decarboxylase | 1.1564796 | 0.9782927 | 1.0409371 | 0.8948768 | 0.8205844 | 0.9427245 |
| Zinc finger protein | 0.8719644 | 0.9578004 | 1.0957803 | 0.8991525 | 1.2171284 | 1.0234778 |
| CDK108 | 0.9250045 | 0.9278348 | 1.2341365 | 1.1054887 | 0.9809254 | 0.7918075 |
| Phase-1 RCT-117 | 1.7347238 | 1.7165619 | 1.287807 | 1.3236941 | 1.2133511 | 1.1626661 |
| {Phase-1 RCT 98} | 1.1150512 | 1.024894 | 1.0399411 | 0.9862378 | 0.9647739 | 0.9340025 |
| Aquaporin-3 (AQP3) | 0.8187251 | 0.7813461 | 0.9689041 | 0.967064 | 0.9696411 | 0.9892083 |
| Cholesterol esterase | 1.2044059 | 1.6297673 | 1.4920088 | 1.1865833 | 1.0251044 | 0.510035 |
| Ribosomal protein S17 | 0.9380409 | 1.0682522 | 1.0522528 | 1.0414035 | 1.4119933 | 1.3039825 |
| Poly(ADP-ribose) polymerase | 0.8147417 | 1.0504817 | 0.9073787 | 0.8445963 | 0.9417154 | 0.9444118 |
| Annexin V | 0.9792886 | 1.1102159 | 0.9343134 | 0.9441067 | 0.905848 | 1.0111622 |
| Phase-1 RCT-33 | 1.053354 | 0.8871756 | 1.047625 | 1.036781 | 0.9533601 | 1.46688 |
| Phase-1 RCT-61 | 0.7690275 | 0.8586264 | 0.8484219 | 0.792423 | 0.9876584 | 1.0264672 |
| Retinol dehydrogenase type III | 1.0201948 | 1.1335521 | 1.1132063 | 1.2856058 | 0.9670559 | 1.0258584 |
| ATPase inhibitor (rat mitochondrial IF1 protein) | 0.9400864 | 1.1516211 | 1.0524501 | 1.2756629 | 1.3856211 | 1.2045159 |
| Thymidylate synthase | 0.9733288 | 1.3294877 | 1.058956 | 0.8683172 | 0.8883226 | 1.0534143 |
| Interleukin-18 | 0.8019382 | 0.9890761 | 0.9870444 | 0.966803 | 1.1107006 | 1.1346093 |
| Lecithin: cholesterol acyltransferase | 1.3592429 | 1.1050256 | 0.9930476 | 1.265632 | 1.1602951 | 0.9159356 |
| Contrapsin-like protease inhibitor (CPi-21) | 1.5685295 | 0.9711784 | 1.5010462 | 1.9608271 | 0.8506722 | 0.8947504 |
| Proliferating cell nuclear antigen gene | 0.8373706 | 0.9234343 | 0.8689663 | 0.8519747 | 0.9964739 | 0.9832836 |
| Phase-1 RCT-230 | 0.7469534 | 0.7146993 | 0.7601582 | 0.7231069 | 0.8353342 | 0.9753009 |
| Cytochrome P450 2D18 | 1.0623835 | 1.0770162 | 1.1523958 | 1.0731335 | 0.7714122 | 0.6510719 |
| Phase-1 RCT-48 | 2.0465758 | 1.2839134 | 1.1361322 | 1.0296986 | 0.8481712 | 0.7504509 |
| Phase-1 RCT-292 | 1.1873741 | 0.8832179 | 0.9482248 | 0.9488217 | 0.9595738 | 0.7978305 |
| Arginosuccinate synthetase 1 | 1.979805 | 0.8644566 | 0.8948703 | 1.0443792 | 1.2455838 | 1.0083305 |
| C-reactive protein | 1.9616205 | 1.7033709 | 1.5059131 | 1.8174131 | 0.8996933 | 0.6925049 |

| | PEG | PHEN | PHEN | PHEN | PHEN | PHEN | PHEN |
|---|---|---|---|---|---|---|---|
| Compound-Dose | 5000 | 20 | 20 | 20 | 80 | 80 | 80 |
| Animal Number | 149 | 1327 | 1328 | 1329 | 1337 | 1338 | 1339 |
| Liver Toxicity Necrosis Classification | no | no | no | no | no | no | no |
| Gene Name | | | | | | | |
| Osteoactivin | 1.0289034 | 0.9500734 | 0.8434394 | 0.9710413 | 0.7396565 | 1.0156243 | 1.1085626 |
| Stathmin | 1.0182971 | 0.8641033 | 1.4148134 | 1.4152936 | 1.1163179 | 1.4786596 | 1.0332574 |
| Calpactin I heavy chain | 1.0278369 | 0.767691 | 0.8534544 | 1.4140733 | 1.0173007 | 1.2143031 | 0.9946806 |
| Phase-1 RCT-179 | 1.1192511 | 0.878417 | 0.913546 | 0.9333717 | 0.8226535 | 0.9390476 | 1.008139 |
| Phase-1 RCT-107 | 1.1955937 | 0.9446107 | 0.7158433 | 0.8263736 | 1.1689981 | 1.1945151 | 2.641321 |
| Heme oxygenase | 1.0473125 | 1.1485046 | 1.6164286 | 2.149804 | 4.180962 | 3.4831421 | 2.9883676 |
| Collagen type II | 1.349897 | 0.7634414 | 0.9263607 | 1.1590472 | 1.2148036 | 1.1163406 | 1.0059994 |
| Phase-1 RCT-81 | 1.014904 | 1.1904075 | 0.9294847 | 0.761147 | 0.7607056 | 0.7984006 | 0.9344303 |
| Glycine methyltransferase | 1.872547 | 0.9011123 | 0.5967125 | 0.7930332 | 1.3271161 | 1.23759 | 3.333004 |
| Cystatin C | 0.9977132 | 0.9662854 | 1.2283502 | 0.8940858 | 0.8087543 | 0.8861446 | 0.9087757 |
| Melanoma-associated antigen ME491 | 1.2330344 | 0.9556993 | 1.0527531 | 0.9669167 | 1.3292792 | 1.4894618 | 1.4640989 |
| Phase-1 RCT-146 | 1.0360625 | 0.9923474 | 1.9554602 | 1.1738262 | 1.0124935 | 1.3903377 | 1.0022497 |
| Gamma-actin, cytoplasmic | 0.7505556 | 1.43216 | 1.067385 | 0.9008073 | 1.0899746 | 0.9382038 | 0.8846059 |
| Adenine nucleotide translocator 1 | 1.0651832 | 0.6901957 | 0.7920063 | 0.7698883 | 0.5659502 | 0.7326478 | 0.8672154 |
| Thymosin beta-10 | 1.1494175 | 0.880469 | 0.6787622 | 0.8202964 | 0.930448 | 0.8434088 | 1.0648799 |
| Insulin-like growth factor I, exon 6 | 1.2020339 | 1.7776401 | 0.8562722 | 0.9975596 | 1.36667 | 1.2900769 | 1.6802658 |
| Phase-1 RCT-78 | 0.8792312 | 1.0807979 | 1.1315683 | 1.1083994 | 1.063137 | 1.2491162 | 1.3246735 |
| Alpha-2-microglobulin | 1.1227499 | 0.6409916 | 0.7204987 | 0.833751 | 0.380384 | 0.5452228 | 0.7282504 |
| High affinity IgE receptor gamma chain | 1.1379485 | 0.8168951 | 1.2744132 | 0.7075291 | 0.8302208 | 0.6757565 | 0.8079093 |
| (FcERIgamma) | | | | | | | |
| 60S ribosomal protein L6 | 1.0983227 | 1.044822 | 0.9631753 | 0.9668061 | 1.0022094 | 0.9656165 | 1.0649172 |
| IgE binding protein | 1.1679251 | 0.9235202 | 0.8165437 | 1.1286193 | 1.3228652 | 1.3914987 | 1.3185848 |
| Phase-1 RCT-149 | 0.746019 | 0.7981218 | 0.9415221 | 1.0476595 | 0.91324 | 0.8326026 | 1.0007408 |
| Cytochrome P450 2C11 | 1.1436024 | 1.1970403 | 2.0691826 | 0.9281887 | 0.9391282 | 1.3219451 | 0.9399312 |
| Uncoupling protein 2 | 1.0753398 | 0.767917 | 0.984815 | 1.147932 | 1.5711931 | 1.5689759 | 1.4106271 |
| Cofilin | 1.025707 | 1.2395713 | 0.8927193 | 1.0398918 | 0.8318366 | 0.9820516 | 1.1515704 |
| Alpha-tubulin | 0.8463656 | 1.2350819 | 1.156562 | 1.0152532 | 1.2042309 | 1.0061581 | 0.9993583 |
| Stearyl-CoA desaturase, liver | 1.6843957 | 1.7476957 | 1.1558585 | 1.2630064 | 0.2673704 | 0.5126047 | 0.4904944 |
| Glutathione S-transferase theta-1 | 0.9124935 | 0.9320768 | 1.0388012 | 0.9053789 | 0.8135014 | 0.7703828 | 0.854183 |
| alpha-1,2-fucosyltrans-ferase | 0.9985345 | 0.7616518 | 0.7199772 | 1.150677 | 1.0059587 | 0.9906598 | 0.9724857 |
| Phase-1 RCT-138 | 1.0646496 | 1.1868455 | 1.0029787 | 0.9563789 | 1.4318782 | 1.3991655 | 1.3758539 |
| Phase-1 RCT-295 | 1.1616896 | 0.7220368 | 0.9515249 | 1.3906136 | 0.9853369 | 1.2208433 | 1.1289216 |
| Phase-1 RCT-164 | 1.1593385 | 0.8670247 | 0.9407809 | 0.7993289 | 0.6212537 | 0.662959 | 0.8654292 |
| Alpha-2-macroglobulin, sequence 2 | 1.1280801 | 1.2818588 | 1.2863355 | 0.8083918 | 0.7333328 | 0.7669066 | 0.7473969 |
| Biliverdin reductase | 0.8801577 | 0.8117963 | 1.2960569 | 1.422719 | 1.4536258 | 1.8290488 | 1.4036462 |
| Phase-1 RCT-121 | 1.0685014 | 1.0478673 | 1.0239031 | 1.0190455 | 1.26616 | 1.2002255 | 1.2010427 |
| Dynamin-1 (D100) | 1.0893624 | 0.9841706 | 1.1265239 | 0.9417111 | 1.1565559 | 1.0272273 | 1.0931138 |
| Phase-1 RCT-192 | 1.1018654 | 0.9080533 | 0.8787786 | 0.951603 | 0.7743031 | 1.0120245 | 1.0865623 |
| Fatty acid synthase | 1.5809014 | 1.5656865 | 1.0517101 | 0.5622095 | 0.3794243 | 0.6037011 | 0.6150593 |
| Phase-1 RCT-211 | 1.08233 | 0.643942 | 0.8885643 | 1.1163471 | 0.9807769 | 1.0627056 | 0.9938509 |
| Ribosomal protein S9 | 1.0453675 | 0.7351016 | 0.9946228 | 0.773369 | 0.613982 | 0.6243242 | 0.6676486 |
| NADP-dependent isocitrate dehydrogenase, | 1.2167779 | 1.0292375 | 0.971504 | 0.853093 | 0.8118866 | 0.795132 | 0.9125444 |
| cytosolic | | | | | | | |
| Phase-1 RCT-57 | 0.8853091 | 0.7697977 | 0.8149334 | 1.5074744 | 1.3632966 | 1.8929595 | 2.0311847 |
| Phase-1 RCT-36 | 1.0343978 | 1.0912681 | 1.1212748 | 1.1998808 | 1.0674257 | 1.2063584 | 1.1354412 |
| Apolipoprotein All | 1.3810918 | 0.6021411 | 0.6636146 | 1.1636549 | 0.8800998 | 0.8913027 | 1.3251402 |
| Phase-1 RCT-68 | 0.9425575 | 1.0279106 | 0.9839419 | 1.2339146 | 1.0290941 | 1.0609816 | 1.0468402 |
| Multidrug resistant protein-2 | 0.6561704 | 0.8294577 | 1.1411386 | 1.070072 | 1.17447 | 1.397472 | 0.995288 |
| Phase-1 RCT-39 | 1.0133487 | 0.8974478 | 1.2369149 | 1.4162961 | 1.4377313 | 1.3780802 | 1.044143 |
| NIPK | 1.0437344 | 1.3450806 | 0.9720427 | 1.0358187 | 1.2838677 | 1.2591583 | 1.5744765 |
| F1-ATPase beta subunit | 0.9725403 | 1.6314552 | 0.8310183 | 0.7976673 | 0.6504256 | 0.6010033 | 0.8091218 |
| Elongation factor-1 alpha | 1.1281983 | 1.3401017 | 0.7678789 | 0.6398962 | 1.1014754 | 0.7442069 | 1.108323 |
| Phase-1 RCT-24 | 0.8831043 | 0.8936416 | 0.8445052 | 1.3789676 | 1.3012017 | 1.6399678 | 1.2235484 |
| Beta-tubulin, class I | 0.6899631 | 0.759272 | 0.8807753 | 1.1461662 | 0.9365943 | 1.1771588 | 0.9692588 |
| 14-3-3 zeta | 0.8205252 | 0.8396151 | 1.3139108 | 1.0135484 | 0.8058768 | 1.3258454 | 1.0111315 |
| Alpha-prothymosin | 1.0192277 | 0.5096754 | 0.8360377 | 1.9331552 | 0.8432443 | 1.4606874 | 1.5061911 |
| Phase-1 RCT-58 | 0.7804207 | 0.7970381 | 0.898784 | 0.6554292 | 0.5790879 | 0.7944019 | 1.3256979 |
| Phase-1 RCT-290 | 1.0902594 | 0.8453279 | 0.5615045 | 1.2018715 | 0.5294412 | 0.7961204 | 1.6232878 |
| Transitional endoplasmic reticulum ATPase | 0.8821053 | 1.3698071 | 1.0718691 | 0.943776 | 0.9351051 | 0.8725734 | 0.9653198 |
| Beta-actin | 0.5518305 | 0.7912191 | 0.6269113 | 0.9326022 | 1.2423221 | 1.1812772 | 1.6395159 |
| Thioredoxin-1 (Trx1) | 1.0553297 | 0.6360307 | 0.84276 | 0.9321508 | 0.8967713 | 0.9633403 | 1.0028462 |
| Phase-1 RCT-174 | 0.9842758 | 1.0903432 | 1.0723261 | 1.0871013 | 0.7646036 | 0.7893791 | 0.8112493 |
| Beta-actin, sequence 2 | 0.8374724 | 1.1301892 | 0.8552977 | 1.0644183 | 0.8207654 | 0.9043374 | 1.1818012 |
| Phase-1 RCT-109 | 1.0190637 | 0.9269736 | 0.8441471 | 0.9931528 | 1.0371422 | 1.0303215 | 1.1016867 |
| Phase-1 RCT-154 | 1.0621507 | 0.8844948 | 1.2545762 | 1.036023 | 1.0234758 | 0.9834861 | 0.8718742 |
| Hypoxanthine-guanine | 0.8585377 | 0.9635972 | 0.8828099 | 1.2323058 | 1.0391074 | 1.2490537 | 1.2742615 |
| phosphoribosyltransferase | | | | | | | |
| Phase-1 RCT-213 | 0.9552508 | 1.0926272 | 1.1859689 | 1.2281487 | 1.1143667 | 1.2658327 | 1.0469623 |
| Phase-1 RCT-260 | 1.0451534 | 0.9830123 | 1.166041 | 1.4077572 | 1.1576518 | 1.1234499 | 0.9033127 |
| Phase-1 RCT-12 | 0.7730046 | 0.8156819 | 0.9187664 | 1.1652293 | 0.9605066 | 1.2350024 | 1.0139291 |
| Phase-1 RCT-293 | 1.0829529 | 0.9296207 | 0.8863475 | 0.9291803 | 1.3984697 | 1.2163569 | 1.3486031 |
| Ribosomal protein S8 | 0.9850563 | 1.0126342 | 1.095618 | 1.2362397 | 1.3873304 | 1.2352511 | 1.360489 |
| Pyruvate kinase, muscle | 0.9517863 | 0.8331975 | 0.8825668 | 1.0205706 | 1.091958 | 1.0736831 | 1.0974643 |
| Nucleoside diphosphate kinase beta isoform | 0.9403613 | 0.6991579 | 0.7250822 | 0.9815806 | 0.6957271 | 0.7914571 | 0.8675094 |
| p55CDC | 0.7881348 | 0.8389983 | 1.3508259 | 0.86003 | 1.0635991 | 1.1836067 | 0.9787451 |
| Phase-1 RCT-156 | 0.7101352 | 1.5183182 | 0.9132113 | 0.8128362 | 0.9138932 | 0.7985027 | 0.9690437 |
| Ribosomal protein L13A | 1.0510298 | 0.6873503 | 0.6946729 | 1.0488769 | 1.1773363 | 1.1972297 | 1.3807391 |
| Phase-1 RCT-258 | 1.0664152 | 1.2240307 | 1.1043986 | 0.9176316 | 1.1826781 | 1.1132804 | 1.0918705 |
| Insulin-like growth factor I | 0.8956096 | 1.554046 | 0.8864366 | 0.6377584 | 0.6425217 | 0.5607445 | 0.67383 |
| Cytochrome P450 3A1 | 0.9622343 | 1.4735456 | 0.734779 | 0.6638278 | 1.5173103 | 0.8138249 | 1.1885111 |
| {Ribosomal protein 16} | 1.0394444 | 1.0096154 | 0.9804236 | 1.4320704 | 1.1507964 | 1.1481127 | 1.2421256 |
| Organic cation transporter 3 | 1.0557235 | 1.0338523 | 0.9445524 | 0.9994529 | 0.9976162 | 0.9976416 | 1.0574248 |
| Calpain 2 | 1.0060794 | 0.9791532 | 1.0812542 | 1.2394491 | 1.1848758 | 1.2934686 | 1.1743277 |
| Phase-1 RCT-102 | 0.8965221 | 1.1044443 | 1.1378043 | 0.939426 | 0.682303 | 0.9019616 | 0.95261 |
| Equilbrative nitrobenzylthioinosine-sensitive | 1.1563189 | 0.9828767 | 0.8898659 | 0.6990802 | 0.7413718 | 0.6494108 | 0.692866 |
| nucleoside transporter | | | | | | | |
| Phase-1 RCT-227 | 1.2696497 | 1.0110458 | 1.1197497 | 1.159399 | 1.01957 | 1.2690349 | 1.3559684 |
| N-hydroxy-2-acetylaminofluorene sulfotransferase (ST1C1) | 0.8456219 | 1.0325595 | 0.9087158 | 0.5027654 | 0.5364251 | 0.5901848 | 0.6797214 |
| Cathepsin S | 1.094383 | 1.1169642 | 1.2224767 | 0.7571469 | 1.135351 | 0.7541432 | 1.0189323 |
| Apolipoprotein CIII | 1.1406921 | 1.1026682 | 1.1810222 | 0.829471 | 0.8878457 | 0.8725669 | 0.8507345 |
| Cytochrome P450 2A3 | 0.9647237 | 1.3388579 | 1.5083386 | 0.747349 | 1.2286944 | 0.8806366 | 1.0232306 |
| Cytochrome P450 2C23 | 1.2861823 | 1.1580917 | 0.9950424 | 1.0221078 | 0.7225106 | 0.903987 | 1.0673525 |
| Apolipoprotein C1 | 0.9456079 | 0.836313 | 1.3497448 | 0.5170944 | 0.5883837 | 0.3723785 | 0.6056793 |
| Betaine homocysteine methyltransferase (BHMT) | 1.1407607 | 0.840445 | 0.6281415 | 1.1755371 | 0.5948758 | 0.8299816 | 1.5356477 |
| Paraoxonase 1 | 0.9998691 | 2.189427 | 1.2371567 | 0.5499833 | 0.8105775 | 0.5350543 | 0.6437017 |
| Phase-1 RCT-207 | 0.9677212 | 0.9564756 | 1.2306519 | 1.1014919 | 1.0934129 | 1.2990397 | 1.0815156 |
| Cathepsin B | 1.0195094 | 1.4336686 | 0.8269861 | 0.7147421 | 1.3668835 | 0.9326986 | 1.169599 |
| Phase-1 RCT-144 | 0.9425872 | 0.9776388 | 1.0940908 | 0.9177287 | 0.9054899 | 1.0146012 | 0.9340063 |
| Alpha-1 microglobulin/bikunin precursor (Ambp) | 1.1076173 | 1.2322185 | 0.9077784 | 0.7694507 | 0.720576 | 0.7951389 | 0.9435417 |
| Cyclin D1 | 1.1350865 | 1.0115763 | 1.0591018 | 0.5174467 | 0.6548554 | 1.6550831 | 0.7427808 |
| Presenilin-1 | 0.6799074 | 2.8712456 | 1.1700162 | 0.4148644 | 0.7307056 | 0.8385416 | 0.9766175 |
| Protein tyrosine phosphatase, receptor type, D | 0.989796 | 0.9579462 | 0.9772212 | 0.5435212 | 0.6729661 | 0.4941345 | 0.5881404 |
| Multidrug resistant protein-1 | 0.6347026 | 0.743039 | 1.0733379 | 1.2291089 | 1.3661746 | 1.3389136 | 1.0889814 |
| Cathepsin L, sequence 2 | 1.0556489 | 0.8380027 | 0.9487014 | 1.0776591 | 0.9388183 | 1.0133058 | 1.0210613 |
| JNK1 stress activated protein kinase | 1.0660213 | 1.1860031 | 1.0454952 | 0.5866502 | 1.0175093 | 0.6970788 | 0.8419678 |
| Phase-1 RCT-43 | 0.7792774 | 1.2461485 | 0.9250667 | 0.8946984 | 0.9309037 | 0.7791606 | 0.8632508 |
| Tissue plasminogen activator | 0.9801409 | 0.963293 | 0.7167196 | 1.1304954 | 0.8114285 | 0.9121836 | 0.9176928 |
| Ubiquitin conjugating enzyme (RAD 6 | 1.0378011 | 0.8226929 | 1.0023693 | 0.7316123 | 0.5889649 | 0.6240776 | 0.7091354 |
| homologue) | | | | | | | |
| Phase-1 RCT-173 | 0.8574911 | 0.9295115 | 1.2112273 | 1.104633 | 1.059144 | 1.1023242 | 1.0016954 |
| Ornithine decarboxylase | 0.8493784 | 0.647416 | 1.1015438 | 1.3316555 | 1.0104629 | 1.9809328 | 1.3190329 |
| Zinc finger protein | 1.0635189 | 1.0276921 | 0.9406398 | 1.0540551 | 0.8158645 | 0.861011 | 1.1003344 |
| CDK108 | 0.8822965 | 0.7407461 | 0.9209732 | 1.1788285 | 0.9627789 | 1.2486589 | 0.9827473 |
| Phase-1 RCT-117 | 1.2707534 | 0.9195736 | 0.7391862 | 1.3817134 | 0.7957573 | 1.0485245 | 0.9929332 |
| {Phase-1 RCT 98} | 0.9585893 | 1.2060589 | 0.8741447 | 0.8696716 | 0.8490261 | 0.8974336 | 0.9671729 |
| Aquaporin-3 (AQP3) | 0.9813522 | 0.9864882 | 1.1888489 | 1.1968999 | 1.0089343 | 1.27768 | 1.2750177 |
| Cholesterol esterase | 0.5544984 | 1.5080776 | 0.7683798 | 0.6572022 | 1.004131 | 0.6837363 | 1.2933774 |
| Ribosomal protein S17 | 1.1545619 | 1 | 0.9253602 | 0.959139 | 0.9013101 | 0.9290698 | 0.9955127 |
| Poly(ADP-ribose) polymerase | 0.8703224 | 0.8970761 | 1.1338184 | 0.9902797 | 0.9451737 | 0.9626401 | 0.8511589 |
| Annexin V | 0.9387036 | 1.0677147 | 1.2797171 | 0.8998813 | 0.9501181 | 0.9213682 | 0.7817976 |
| Phase-1 RCT-33 | 1.1180042 | 1.2728882 | 1.0490067 | 1.2180637 | 1.0483227 | 1.1998255 | 1.2127491 |
| Phase-1 RCT-61 | 0.9828883 | 0.8525206 | 0.9091899 | 0.7108266 | 0.6044076 | 0.8459127 | 1.1620815 |
| Retinol dehydrogenase type III | 1.2182301 | 0.8799918 | 0.8718934 | 0.7035898 | 0.6049579 | 0.5620342 | 0.759127 |
| ATPase inhibitor (rat mitochondrial IF1 protein) | 1.4439021 | 1.0374868 | 0.9026839 | 0.5811643 | 0.5667692 | 0.4745976 | 0.6829346 |
| Thymidylate synthase | 0.9362826 | 0.8992364 | 1.9247 | 1.0930145 | 1.1190742 | 1.4302899 | 0.8945841 |
| Interleukin-18 | 1.0931238 | 1.0684025 | 1.7537163 | 1.0584968 | 1.3890294 | 1.2201375 | 0.9801465 |
| Lecithin: cholesterol acyltransferase | 1.2239821 | 0.9000787 | 0.7796223 | 0.9536482 | 0.8225337 | 0.8327223 | 0.9784941 |
| Contrapsin-like protease inhibitor (CPi-21) | 0.8859232 | 0.9109203 | 0.720101 | 1.155535 | 0.7458385 | 0.892872 | 0.8561586 |
| Proliferating cell nuclear antigen gene | 1.00896 | 1.020274 | 1.7139033 | 0.925612 | 1.133122 | 1.2807806 | 0.9592966 |
| Phase-1 RCT-230 | 0.9607096 | 0.9348319 | 1.3900096 | 1.1013391 | 1.4236934 | 1.3708019 | 1.0212104 |
| Cytochrome P450 2D18 | 0.658903 | 0.6977887 | 0.7779856 | 1.7464085 | 1.5186303 | 2.2032201 | 2.5513394 |
| Phase-1 RCT-48 | 0.9982773 | 0.7736548 | 0.8951057 | 1.3786862 | 0.8827195 | 0.942738 | 1.206217 |
| Phase-1 RCT-292 | 0.9422234 | 1.0790194 | 0.9975053 | 1.011346 | 0.9296608 | 0.9462437 | 0.9635879 |
| Arginosuccinate synthetase 1 | 1.255965 | 1.2759048 | 0.9833063 | 1.1717914 | 0.8748613 | 1.018888 | 1.1449914 |
| C-reactive protein | 0.9291207 | 0.5526388 | 0.7796146 | 0.438216 | 0.3770891 | 0.4115631 | 0.5066624 |

| | PUR | PUR | PUR | PUR | PUR | PUR |
|---|---|---|---|---|---|---|
| Compound-Dose | 38 | 38 | 38 | 150 | 150 | 150 |
| Animal Number | 27 | 28 | 29 | 37 | 38 | 39 |
| Liver Toxicity Necrosis Classification | no | no | no | no | no | no |
| Gene Name | | | | | | |
| Osteoactivin | 1.1582068 | 1.2378268 | 1.0670835 | 1.1118499 | 1.0224291 | 1.0411617 |
| Stathmin | 1.0845861 | 0.7459799 | 0.9729928 | 0.9802951 | 0.8153869 | 1.1420785 |
| Calpactin I heavy chain | 1.1399964 | 1.208268 | 2.18831 | 1.0694839 | 1.0849239 | 0.9473743 |
| Phase-1 RCT-179 | 0.9608498 | 0.8295623 | 1.0237348 | 1.1540234 | 1.0219029 | 1.1934485 |
| Phase-1 RCT-107 | 1.0183531 | 1.0666618 | 0.871295 | 0.8260652 | 1.0469544 | 1.4868251 |
| Heme oxygenase | 0.9218939 | 0.7883704 | 0.8727841 | 0.9446513 | 0.7951127 | 1.0520005 |
| Collagen type II | 0.6464853 | 0.6628525 | 0.8683935 | 0.9571652 | 1.0560583 | 1.1351736 |
| Phase-1 RCT-81 | 1.5260962 | 1.7599847 | 1.441407 | 1.352215 | 1.4763887 | 1.4186684 |
| Glycine methyltransferase | 1.1337544 | 1.1875312 | 0.8234666 | 0.8900344 | 1.1263224 | 0.7530536 |
| Cystatin C | 1.2226878 | 1.2173318 | 1.1449918 | 1.0688258 | 1.052538 | 1.1829597 |
| Melanoma-associated antigen ME491 | 0.9949661 | 1.1209311 | 1.1097815 | 0.9607616 | 0.9904521 | 0.997305 |
| Phase-1 RCT-146 | 0.8980839 | 0.7836394 | 0.9255841 | 0.9304407 | 0.9508618 | 1.0312347 |
| Gamma-actin, cytoplasmic | 1.355279 | 1.0763612 | 0.8965104 | 0.9658777 | 0.9414952 | 0.8475699 |
| Adenine nucleotide translocator 1 | 0.6415393 | 0.7013545 | 0.8657838 | 1.1306182 | 0.9369251 | 1.1067758 |
| Thymosin beta-10 | 1.2834848 | 1.2218758 | 1.0187026 | 1.2908969 | 0.9317654 | 1.2107594 |
| Insulin-like growth factor I, exon 6 | 1.3330091 | 0.864947 | 0.9338943 | 0.8849847 | 0.7737454 | 0.8067661 |
| Phase-1 RCT-78 | 0.9934394 | 1.200883 | 1.0140277 | 0.7710784 | 1.0635295 | 0.7660577 |
| Alpha-2-microglobulin | 0.735419 | 1.1202011 | 0.7635009 | 0.7570139 | 0.7757962 | 0.6622604 |
| High affinity IgE receptor gamma chain | 0.8469642 | 0.8610163 | 1.0731874 | 0.9333382 | 0.8986077 | 0.6593808 |
| (FcERIgamma) | | | | | | |
| 60S ribosomal protein L6 | 1.2370393 | 1.2231554 | 1.1317111 | 1.2503052 | 1.042019 | 1.3447319 |
| IgE binding protein | 1.0909609 | 1.2609106 | 1.0276891 | 0.9291205 | 0.814232 | 0.7649192 |
| Phase-1 RCT-149 | 0.9505655 | 0.9760387 | 0.9868511 | 0.8799561 | 0.920596 | 0.9026585 |
| Cytochrome P450 2C11 | 0.9673684 | 1.1497266 | 1.4182663 | 1.017046 | 1.1291597 | 1.0730568 |
| Uncoupling protein 2 | 1.2437594 | 1.1540362 | 1.1462759 | 1.2764351 | 0.9874833 | 1.2237755 |
| Cofilin | 0.9823412 | 1.1037228 | 0.9760072 | 0.7791025 | 0.6416411 | 0.5754041 |
| Alpha-tubulin | 0.7300552 | 0.7783041 | 1.2666655 | 1.2002424 | 0.9943005 | 1.1848084 |
| Stearyl-CoA desaturase, liver | 0.5634288 | 0.7088478 | 0.9192585 | 0.2074664 | 0.3776468 | 0.4761107 |
| Glutathione S-transferase theta-1 | 0.9293647 | 1.297035 | 1.1155844 | 1.0444516 | 1.0778705 | 1.0022807 |
| alpha-1,2-fucosyltrans-ferase | 1.8501295 | 1.5016695 | 1.346286 | 1.5406592 | 1.4957635 | 1.5736496 |
| Phase-1 RCT-138 | 1.0207037 | 1.0620667 | 1.0877975 | 1.0134463 | 1.0704437 | 1.0406088 |
| Phase-1 RCT-295 | 1.3633182 | 1.3280154 | 1.265125 | 1.336384 | 1.3447627 | 1.386468 |
| Phase-1 RCT-164 | 0.9148933 | 0.8836759 | 0.9986116 | 1.0474497 | 0.8834072 | 0.9054821 |
| Alpha-2-macroglobulin, sequence 2 | 1.5983135 | 1.2467886 | 1.1189486 | 1.1550144 | 1.1024181 | 0.9656101 |
| Biliverdin reductase | 0.8066707 | 0.8937793 | 0.9793118 | 1.1563157 | 1.1467459 | 1.3056004 |
| Phase-1 RCT-121 | 0.6478706 | 0.6427892 | 0.7822552 | 0.9468504 | 0.7859862 | 0.9428601 |
| Dynamin-1 (D100) | 1.1832037 | 1.343775 | 1.2038401 | 1.1457201 | 1.0842416 | 0.9736155 |
| Phase-1 RCT-192 | 1.0384403 | 0.9716664 | 0.9828426 | 1.0789226 | 0.9116112 | 0.9989553 |
| Fatty acid synthase | 0.6838145 | 0.9953025 | 0.7656796 | 0.5195299 | 0.7602761 | 0.8211792 |
| Phase-1 RCT-211 | 1.0303864 | 1.1263247 | 1.1265479 | 1.0216662 | 1.0552953 | 0.9146423 |
| Ribosomal protein S9 | 0.940093 | 0.8922199 | 1.109907 | 1.2088898 | 1.1248084 | 1.1693388 |
| NADP-dependent isocitrate dehydrogenase, | 1.1938426 | 1.0700688 | 1.0754646 | 1.0225576 | 0.8990783 | 0.8538594 |
| cytosolic | | | | | | |
| Phase-1 RCT-57 | 1.2691727 | 1.3667741 | 1.1994745 | 1.3955855 | 1.3480895 | 1.3217778 |
| Phase-1 RCT-36 | 1.0911418 | 1.102863 | 1.0023121 | 0.9286835 | 0.8639761 | 0.9357016 |
| Apolipoprotein All | 0.8469037 | 0.6878629 | 0.7727451 | 0.6428627 | 0.6036339 | 0.6100757 |
| Phase-1 RCT-68 | 1.0837033 | 1.1440827 | 1.1570332 | 1.0182993 | 1.1531304 | 1.0504894 |
| Multidrug resistant protein-2 | 0.8518618 | 0.7221483 | 1.067969 | 1.1651609 | 1.2226619 | 1.1739436 |
| Phase-1 RCT-39 | 1.1298518 | 0.9260905 | 0.9322421 | 1.1376967 | 1.0955429 | 1.2027022 |
| NIPK | 0.9812499 | 0.7906796 | 0.9394903 | 0.87725 | 0.7894709 | 0.8427293 |
| F1-ATPase beta subunit | 1.2967168 | 1.6157926 | 1.1105392 | 1.1232966 | 1.2896265 | 1.1705903 |
| Elongation factor-1 alpha | 1.0676872 | 1.2119732 | 0.8983849 | 0.8687106 | 0.9050245 | 0.9104725 |
| Phase-1 RCT-24 | 0.9016449 | 0.9457044 | 0.9871031 | 0.9814012 | 1.0813498 | 1.0550641 |
| Beta-tubulin, class I | 1.2361909 | 1.2436154 | 1.3093246 | 1.4278208 | 1.26129 | 1.3868597 |
| 14-3-3 zeta | 0.8238129 | 0.6124888 | 0.9050912 | 1.0731206 | 0.973581 | 1.1013387 |
| Alpha-prothymosin | 0.9213603 | 1.0548732 | 0.8880039 | 0.6833532 | 0.6320405 | 0.5234571 |
| Phase-1 RCT-58 | 1.0390253 | 0.7468845 | 1.0932161 | 1.2838991 | 0.6849626 | 0.7480927 |
| Phase-1 RCT-290 | 2.093369 | 1.6481699 | 1.1629311 | 1.0589684 | 0.7378559 | 0.7017312 |
| Transitional endoplasmic reticulum ATPase | 0.8179621 | 0.8074789 | 0.9189155 | 0.9900054 | 0.9206598 | 0.9848964 |
| Beta-actin | 1.0390668 | 0.9115635 | 0.7269368 | 1.0112054 | 0.8008941 | 0.6906413 |
| Thioredoxin-1 (Trx1) | 1.3493866 | 1.3738009 | 1.143758 | 1.1883891 | 1.2665077 | 1.1813692 |
| Phase-1 RCT-174 | 0.8324633 | 1.279029 | 1.0570757 | 0.8480532 | 1.1317686 | 1.0481069 |
| Beta-actin, sequence 2 | 1.0309684 | 1.127659 | 0.9773815 | 0.8398216 | 0.8630678 | 0.7455126 |
| Phase-1 RCT-109 | 1.1547353 | 1.2051399 | 1.2816981 | 1.3213788 | 1.2096131 | 1.3474108 |
| Phase-1 RCT-154 | 0.8706316 | 0.8803257 | 0.9121558 | 0.9728951 | 0.7807736 | 0.9694243 |
| Hypoxanthine-guanine | 1.3370832 | 1.3403425 | 1.2674739 | 1.0240201 | 0.8975225 | 1.0326848 |
| phosphoribosyltransferase | | | | | | |
| Phase-1 RCT-213 | 0.7582536 | 0.7642087 | 0.8437048 | 1.0238827 | 0.8908346 | 1.0582463 |
| Phase-1 RCT-260 | 0.9219216 | 0.7716512 | 0.8723719 | 0.935575 | 0.917765 | 0.9608865 |
| Phase-1 RCT-12 | 1.1546273 | 1.1262238 | 1.252319 | 1.3253886 | 1.2130663 | 1.2665112 |
| Phase-1 RCT-293 | 1.5024742 | 1.198498 | 1.2000381 | 1.4789972 | 1.3402593 | 1.3657575 |
| Ribosomal protein S8 | 1.307402 | 1.4015243 | 1.0982655 | 1.148 | 1.0036935 | 1.212705 |
| Pyruvate kinase, muscle | 1.2059336 | 0.9923154 | 0.9947271 | 1.2150221 | 0.9866162 | 1.1319643 |
| Nucleoside diphosphate kinase beta isoform | 1.0609847 | 1.2674762 | 1.1306401 | 1.09877 | 1.0790161 | 1.4567969 |
| p55CDC | 0.741497 | 0.9043712 | 1.122785 | 1.1121769 | 1.2996013 | 1.4052888 |
| Phase-1 RCT-156 | 1.0469619 | 1.1144736 | 1.2219226 | 1.0500057 | 1.1632013 | 1.1726009 |
| Ribosomal protein L13A | 1.5592958 | 1.3942051 | 1.1819656 | 1.3701069 | 1.0624647 | 1.2513981 |
| Phase-1 RCT-258 | 0.8904036 | 0.9484224 | 0.9107782 | 1.1019628 | 0.9704149 | 1.043688 |
| Insulin-like growth factor I | 0.9591691 | 0.8350829 | 0.6981089 | 0.572593 | 0.4925339 | 0.6388749 |
| Cytochrome P450 3A1 | 0.9521074 | 0.7201294 | 0.8300013 | 1.2984931 | 0.9943005 | 0.7592694 |
| {Ribosomal protein 16} | 1.1919409 | 1.365067 | 1.1216959 | 0.8433927 | 0.9164012 | 0.8322156 |
| Organic cation transporter 3 | 1.2080355 | 1.2020062 | 1.127458 | 1.2671131 | 1.0252182 | 1.3371798 |
| Calpain 2 | 1.1880814 | 1.2147379 | 1.168931 | 1.0338526 | 0.9798665 | 1.1298864 |
| Phase-1 RCT-102 | 0.8564128 | 0.8051286 | 0.8270408 | 0.8347714 | 0.7316352 | 0.707724 |
| Equilbrative nitrobenzylthioinosine-sensitive | 0.9183648 | 0.7347441 | 0.8997524 | 1.0231069 | 0.8889274 | 0.9021213 |
| nucleoside transporter | | | | | | |
| Phase-1 RCT-227 | 0.9018074 | 1.4272811 | 1.01869 | 0.704148 | 1.1398218 | 0.7739278 |
| N-hydroxy-2-acetylaminofluorene sulfotransferase (ST1C1) | 1.1434051 | 1.2223645 | 0.9813951 | 1.2205179 | 0.9348475 | 1.0825053 |
| Cathepsin S | 0.8809963 | 0.8732019 | 0.9738257 | 0.947246 | 0.9372252 | 1.1494035 |
| Apolipoprotein CIII | 0.8898597 | 1.001025 | 1.1205373 | 0.878228 | 0.772579 | 0.7462365 |
| Cytochrome P450 2A3 | 1.3031232 | 1.6327293 | 1.9157736 | 1.2730938 | 0.7641947 | 0.7629216 |
| Cytochrome P450 2C23 | 1.1686864 | 0.8273016 | 0.8172485 | 1.0617442 | 0.7065878 | 0.8377543 |
| Apolipoprotein C1 | 0.8333466 | 0.8197776 | 1.0104177 | 0.7885436 | 0.877133 | 0.5511113 |
| Betaine homocysteine methyltransferase (BHMT) | 2.3014543 | 1.7721671 | 1.1815759 | 1.0198315 | 0.7617211 | 0.708125 |
| Paraoxonase 1 | 0.9667567 | 1.092633 | 0.8213946 | 0.7415383 | 0.765952 | 0.9024116 |
| Phase-1 RCT-207 | 1.2777399 | 0.7322838 | 0.8810274 | 1.0633217 | 0.9514113 | 1.0384041 |
| Cathepsin B | 1.1724919 | 1.2499205 | 1.0102451 | 0.958917 | 1.1166619 | 1.0346282 |
| Phase-1 RCT-144 | 0.9090613 | 0.8878967 | 1.0380138 | 1.1121991 | 1.1004653 | 1.0910759 |
| Alpha-1 microglobulin/bikunin precursor (Ambp) | 1.549811 | 1.7922391 | 1.4725221 | 1.3794248 | 1.4971727 | 1.4347867 |
| Cyclin D1 | 0.8576438 | 0.7731614 | 1.1270787 | 1.6311865 | 1.1043688 | 1.5152342 |
| Presenilin-1 | 0.8171002 | 0.7293739 | 0.8733891 | 0.4830053 | 0.4399568 | 0.4946043 |
| Protein tyrosine phosphatase, receptor type, D | 1.0640041 | 1.1748524 | 0.821158 | 0.8389864 | 0.9111501 | 0.8967124 |
| Multidrug resistant protein-1 | 0.7471545 | 0.7891682 | 1.0102626 | 1.0469954 | 1.23671 | 1.1489909 |
| Cathepsin L, sequence 2 | 1.3513954 | 1.020014 | 1.000269 | 1.1645195 | 0.9089876 | 0.9899586 |
| JNK1 stress activated protein kinase | 1.0492048 | 1.3084962 | 0.9194116 | 0.9341751 | 0.8389974 | 0.8133 |
| Phase-1 RCT-43 | 0.8508316 | 0.8576536 | 0.9004657 | 0.9120678 | 0.8135497 | 0.7831045 |
| Tissue plasminogen activator | 1.317346 | 1.6415704 | 1.0569253 | 1.1177018 | 1.0212727 | 1.1875848 |
| Ubiquitin conjugating enzyme (RAD 6 | 1.0002911 | 0.8466367 | 1.0416236 | 1.1770309 | 1.0510105 | 1.2510171 |
| homologue) | | | | | | |
| Phase-1 RCT-173 | 0.6965799 | 0.8279744 | 0.9205803 | 0.9641349 | 0.8877454 | 1 |
| Ornithine decarboxylase | 0.8325827 | 0.7489302 | 0.8687818 | 1.0407149 | 0.8794793 | 0.981844 |
| Zinc finger protein | 0.6431108 | 0.6004228 | 0.8158994 | 0.8214053 | 0.7578651 | 0.8787563 |
| CDK108 | 1.0215291 | 1.0144691 | 1.1491207 | 1.0795531 | 1.083368 | 0.9576038 |
| Phase-1 RCT-117 | 1.1428003 | 1.6783105 | 1.3067242 | 1.2918054 | 1.3032497 | 1.2068608 |
| {Phase-1 RCT 98} | 1.2351733 | 1.3398191 | 1.1483765 | 0.934557 | 1.1274976 | 0.8767912 |
| Aquaporin-3 (AQP3) | 1.0275537 | 0.9749036 | 1.013484 | 0.8382462 | 1.0349002 | 0.8199887 |
| Cholesterol esterase | 0.9207993 | 0.9722043 | 1.3914382 | 1.2297682 | 1.2638898 | 0.7905415 |
| Ribosomal protein S17 | 1.5229725 | 1.5909503 | 1.1475215 | 1.4084346 | 1.3060691 | 1.4854932 |
| Poly(ADP-ribose) polymerase | 0.9947692 | 0.9946399 | 0.9858599 | 0.9074903 | 0.8737869 | 0.9390088 |
| Annexin V | 0.9995727 | 0.9814941 | 1.092444 | 1.3679475 | 1.1459996 | 1.2141554 |
| Phase-1 RCT-33 | 1.0939077 | 1.090060 | 0.9917615 | 0.9826965 | 0.9167703 | 0.9877515 |
| Phase-1 RCT-61 | 0.9039533 | 0.7570859 | 0.9195222 | 1.1663343 | 0.8229175 | 0.7448668 |
| Retinol dehydrogenase type III | 1.1660267 | 1.1978148 | 0.9641266 | 0.8483772 | 0.887214 | 0.8468165 |
| ATPase inhibitor (rat mitochondrial IF1 protein) | 1.1646835 | 0.713587 | 0.9010187 | 0.8277928 | 0.6301874 | 0.7423163 |
| Thymidylate synthase | 0.6941422 | 0.7616848 | 0.8738253 | 1.1799157 | 1.2255076 | 1.2490703 |
| Interleukin-18 | 0.996651 | 1.1616725 | 1.0680219 | 1.2114601 | 1.062499 | 1.3449333 |
| Lecithin: cholesterol acyltransferase | 1.2968982 | 1.3278733 | 1.0477657 | 1.1689806 | 1.2489554 | 1.3080289 |
| Contrapsin-like protease inhibitor (CPi-21) | 1.3228254 | 1.7467638 | 1.0318791 | 1.0833223 | 0.9976169 | 1.0835694 |
| Proliferating cell nuclear antigen gene | 0.756029 | 0.8016167 | 1.0207137 | 1.0127549 | 1.070939 | 1.1172235 |
| Phase-1 RCT-230 | 1.0073223 | 0.8271216 | 0.9046579 | 0.9865127 | 0.946827 | 0.9231017 |
| Cytochrome P450 2D18 | 1.6051362 | 1.5869088 | 1.2745472 | 1.4872073 | 1.4803327 | 1.4230059 |
| Phase-1 RCT-48 | 1.3161694 | 1.6231503 | 1.38229 | 1.1207651 | 1.0740082 | 1.1636014 |
| Phase-1 RCT-292 | 0.899762 | 0.8779519 | 0.9182891 | 0.9710051 | 0.9058021 | 0.9483326 |
| Arginosuccinate synthetase 1 | 0.8441646 | 1.0116752 | 0.886862 | 0.6610183 | 0.5762014 | 0.6516972 |
| C-reactive protein | 0.8916302 | 0.9645294 | 0.7416801 | 0.6569314 | 0.5668739 | 0.6221727 |

| | QUIN | QUIN | QUIN | QUIN | QUIN | QUIN | STRZ |
|---|---|---|---|---|---|---|---|
| Compound-Dose | 25 | 25 | 25 | 100 | 100 | 100 | 20 |
| Animal Number | 2547 | 2548 | 2549 | 2557 | 2558 | 2559 | 1727 |
| Liver Toxicity Necrosis Classification | no | no | no | no | no | no | no |
| Gene Name | | | | | | | |
| Osteoactivin | 0.97124 | 1.0061376 | 0.9280635 | 0.9779809 | 1.0351181 | 0.9858047 | 1.0490184 |
| Stathmin | 1.0244119 | 0.9816406 | 0.9505481 | 0.9625592 | 1.0247386 | 0.9737765 | 1.0279847 |
| Calpactin I heavy chain | 1.2772483 | 1.0442889 | 1.1949829 | 1.0275662 | 0.9655668 | 0.9705727 | 1.0818595 |
| Phase-1 RCT-179 | 0.894759 | 0.9816111 | 1.1124415 | 0.9642554 | 1.03769 | 1.0833944 | 1.0135547 |
| Phase-1 RCT-107 | 0.9576957 | 0.9320853 | 1.1492205 | 0.9083678 | 0.9576157 | 0.9573544 | 1.1296818 |
| Heme oxygenase | 0.9951338 | 0.8956893 | 1.2546574 | 1.0665464 | 1.0097294 | 1.0589777 | 1.0266395 |
| Collagen type II | 1.1145421 | 1.0591134 | 1.099322 | 1.0091171 | 0.9705653 | 0.8733599 | 1.0801872 |
| Phase-1 RCT-81 | 0.8848574 | 1.0965097 | 1.120175 | 1.0881356 | 1.0996834 | 0.9226543 | 1.0095319 |
| Glycine methyltransferase | 0.9258677 | 0.9260165 | 1.2728403 | 0.9241019 | 0.9406163 | 1.0522039 | 1.8285745 |
| Cystatin C | 0.8494524 | 1.1330378 | 0.9186944 | 1.0668005 | 1.1172538 | 0.9043927 | 1.3013784 |
| Melanoma-associated antigen ME491 | 0.9058779 | 0.9750289 | 1.0864704 | 0.7817388 | 1.0916529 | 0.9266231 | 1.0497285 |
| Phase-1 RCT-146 | 0.9441484 | 1.0135881 | 0.9499753 | 0.8589929 | 0.9796795 | 0.8998202 | 0.9368497 |
| Gamma-actin, cytoplasmic | 1.4190978 | 0.6678903 | 0.7518075 | 0.7479976 | 0.6068466 | 0.8468062 | 0.8932847 |
| Adenine nucleotide translocator 1 | 0.9652854 | 1.110091 | 1.0536759 | 0.8515291 | 1.0314013 | 0.9750768 | 0.9197627 |
| Thymosin beta-10 | 1.0707306 | 1.0774904 | 1.2270372 | 1.1195964 | 0.9550267 | 0.990432 | 0.9955062 |
| Insulin-like growth factor I, exon 6 | 1.285446 | 0.7834837 | 0.848972 | 1.2487366 | 1.0718781 | 1.3255228 | 0.9953311 |
| Phase-1 RCT-78 | 0.8194279 | 1.0923178 | 0.965971 | 1.0554497 | 1.0257162 | 0.9459599 | 1.0793059 |
| Alpha-2-microglobulin | 0.6038681 | 0.814335 | 0.6024844 | 0.877608 | 0.8561148 | 0.9326257 | 0.836197 |
| High affinity IgE receptor gamma chain | 0.8489114 | 0.891618 | 0.8866505 | 0.893115 | 0.9024923 | 0.8377456 | 1.0762997 |
| (FcERIgamma) | | | | | | | |
| 60S ribosomal protein L6 | 0.8227094 | 1.0527912 | 1.0758228 | 0.8839137 | 1.0392073 | 0.9464871 | 0.9128224 |
| IgE binding protein | 0.9678249 | 0.989683 | 0.9299597 | 0.9534714 | 0.9102276 | 0.9549304 | 0.9665805 |
| Phase-1 RCT-149 | 1.2193501 | 0.8742385 | 0.7736893 | 0.9686423 | 0.995736 | 0.8900275 | 1.3018314 |
| Cytochrome P450 2C11 | 0.5667465 | 0.9851209 | 0.9934037 | 0.8990396 | 0.8945202 | 0.8471327 | 0.7363272 |
| Uncoupling protein 2 | 1.0543463 | 0.9991604 | 1.110257 | 1.04138 | 0.9403235 | 1.0517151 | 1.0614508 |
| Cofilin | 0.929557 | 1.114207 | 1.0610856 | 1.0171806 | 1.0534307 | 0.9439942 | 1.1961728 |
| Alpha-tubulin | 1.175886 | 1.0189536 | 1.1423062 | 0.9885341 | 0.9461383 | 1.0416632 | 1.0472834 |
| Stearyl-CoA desaturase, liver | 2.8752098 | 1.2012873 | 1.7152056 | 1.314773 | 0.6519362 | 0.5686017 | 1.2502089 |
| Glutathione S-transferase theta-1 | 0.8726201 | 1.1257445 | 0.9794065 | 1.6199678 | 1.3282545 | 1.5281242 | 1.2650352 |
| alpha-1,2-fucosyltrans-ferase | 1.06556 | 0.9605911 | 1.0866237 | 1.1445013 | 1.2049645 | 1.0600039 | 0.8766788 |
| Phase-1 RCT-138 | 0.9841272 | 0.9590592 | 0.9556988 | 1.0190032 | 0.895926 | 0.9507688 | 1.2377193 |
| Phase-1 RCT-295 | 1.0071126 | 0.9982767 | 1.1167892 | 1.2895762 | 1.1234297 | 1.038579 | 1.0902256 |
| Phase-1 RCT-164 | 0.9729548 | 1.248504 | 1.2258366 | 1.0511123 | 1.0485944 | 1.077022 | 0.9102571 |
| Alpha-2-macroglobulin, sequence 2 | 0.8235409 | 0.8394044 | 0.8637345 | 0.9016498 | 0.954121 | 0.9472152 | 1.1018524 |
| Biliverdin reductase | 1.060894 | 1.0101516 | 1.0917469 | 0.9856123 | 0.9753521 | 0.9959527 | 0.9464261 |
| Phase-1 RCT-121 | 1.0018616 | 0.9264807 | 0.9150295 | 0.9790869 | 1.1723486 | 0.9868847 | 0.9517392 |
| Dynamin-1 (D100) | 0.956245 | 1.0382197 | 0.9485353 | 1.0593034 | 1.0393091 | 0.9658502 | 1.046778 |
| Phase-1 RCT-192 | 0.771831 | 0.9966442 | 1.0022706 | 0.8807884 | 1.0060437 | 0.8520593 | 0.9611827 |
| Fatty acid synthase | 2.249205 | 0.9769117 | 1.3093882 | 0.8207914 | 0.5617477 | 0.5295592 | 1.4409964 |
| Phase-1 RCT-211 | 1.0239049 | 0.9990471 | 0.9877148 | 1.1635185 | 0.8808618 | 1.0357441 | 1.1626976 |
| Ribosomal protein S9 | 0.9224803 | 1.0465045 | 1.23451 | 1.131252 | 1.1264793 | 0.984407 | 0.93231 |
| NADP-dependent isocitrate dehydrogenase, | 1.0641409 | 1.0908653 | 1.1275893 | 1.1242539 | 1.0193512 | 1.0633465 | 1.0265496 |
| cytosolic | | | | | | | |
| Phase-1 RCT-57 | 1.1173627 | 0.9631972 | 1.0708482 | 1.1296686 | 1.0730482 | 1.1471978 | 1.017943 |
| Phase-1 RCT-36 | 0.97425 | 0.9278935 | 0.9889395 | 1.0122712 | 0.8727641 | 0.897801 | 0.9734428 |
| Apolipoprotein All | 1.1172696 | 1.2497916 | 1.031308 | 1.0499594 | 1.0148792 | 0.9565374 | 0.9210587 |
| Phase-1 RCT-68 | 1.1161436 | 0.9828708 | 1.0767516 | 1.0484443 | 0.9509151 | 0.983961 | 0.9766213 |
| Multidrug resistant protein-2 | 1.0364275 | 1.057535 | 0.9735326 | 1.0921131 | 1.0364594 | 1.5300651 | 0.9449586 |
| Phase-1 RCT-39 | 1.0874932 | 0.9257468 | 1.023615 | 0.9541944 | 0.9150403 | 1.0613635 | 0.9301108 |
| NIPK | 1.2062012 | 0.8373175 | 0.9700717 | 1.1382315 | 1.0284169 | 1.1234214 | 1.0294342 |
| F1-ATPase beta subunit | 1.118846 | 1.0445298 | 1.0976306 | 0.9758936 | 0.9947143 | 1.0098252 | 1.1680216 |
| Elongation factor-1 alpha | 1.0089321 | 1.0850327 | 1.0663947 | 1.059645 | 1.001601 | 1.076179 | 1.1711075 |
| Phase-1 RCT-24 | 1.1883657 | 0.8660076 | 1.1743592 | 0.8839173 | 0.8831902 | 0.9616442 | 1.2509942 |
| Beta-tubulin, class I | 1.1143123 | 0.802021 | 1.2649281 | 0.9371563 | 0.8791301 | 0.8686442 | 1.4117148 |
| 14-3-3 zeta | 1.3605899 | 1.075881 | 1.3362981 | 1.0582266 | 0.9791054 | 1.0598502 | 1.1375136 |
| Alpha-prothymosin | 1.008643 | 1.1579872 | 1.1131417 | 1.0551318 | 1.0957848 | 0.9989584 | 1.2271326 |
| Phase-1 RCT-58 | 1.6264133 | 1.1346253 | 1.4990122 | 1.2741704 | 1.2089409 | 1.3194873 | 1.4701365 |
| Phase-1 RCT-290 | 1.2920642 | 1.2397969 | 0.8959155 | 1.1423194 | 0.8319473 | 0.8060568 | 1.0930523 |
| Transitional endoplasmic reticulum ATPase | 1.1196365 | 0.8987766 | 1.1829739 | 0.9667239 | 0.9778772 | 1.0155425 | 1.0763761 |
| Beta-actin | 2.151073 | 0.6221959 | 1.2349494 | 1.019095 | 0.5054078 | 0.9500377 | 1.495564 |
| Thioredoxin-1 (Trx1) | 0.8324468 | 0.9566965 | 0.9033428 | 1.0207506 | 1.0499303 | 0.9537457 | 0.9546966 |
| Phase-1 RCT-174 | 0.9822114 | 1.0420951 | 1.0200717 | 1.0572126 | 1.1781574 | 0.9806683 | 0.9980291 |
| Beta-actin, sequence 2 | 1.2361727 | 0.9723044 | 1.0433387 | 0.9812766 | 0.8195032 | 0.9281902 | 1.0319701 |
| Phase-1 RCT-109 | 1.0627499 | 0.9864537 | 1.2483494 | 1.1180122 | 0.9772245 | 1.0024496 | 1.009289 |
| Phase-1 RCT-154 | 1.0889157 | 1.073549 | 1.0663593 | 0.9396992 | 0.9888941 | 0.8999308 | 0.9925309 |
| Hypoxanthine-guanine | 1.1663437 | 0.9870846 | 1.2400029 | 1.0147275 | 1.0128499 | 0.9676011 | 1.0925663 |
| phosphoribosyltransferase | | | | | | | |
| Phase-1 RCT-213 | 1.0633751 | 0.9596611 | 1.015372 | 0.9628117 | 0.9720698 | 0.9493774 | 0.9698361 |
| Phase-1 RCT-260 | 0.9290273 | 0.8660047 | 0.8271887 | 0.8113275 | 0.868847 | 0.9066597 | 0.9145643 |
| Phase-1 RCT-12 | 1.2416811 | 0.8673012 | 1.1273302 | 1.0215988 | 0.9531941 | 1.005791 | 1.4545006 |
| Phase-1 RCT-293 | 0.8958671 | 0.9677703 | 0.9735542 | 1.0998509 | 0.9652482 | 0.928076 | 1.0345396 |
| Ribosomal protein S8 | 0.768013 | 0.9260868 | 0.9730623 | 1.0277543 | 1.1363661 | 0.9620209 | 1.0823162 |
| Pyruvate kinase, muscle | 1.022166 | 0.9787921 | 1.0321975 | 0.965848 | 1.0054578 | 1.048399 | 1.1552222 |
| Nucleoside diphosphate kinase beta isoform | 1.0572097 | 1.1019281 | 1.0688126 | 1.0488572 | 1.120888 | 1.0608212 | 1.020418 |
| p55CDC | 0.9453195 | 0.983964 | 0.9177188 | 0.8622025 | 1.0350207 | 1.0278579 | 0.8806158 |
| Phase-1 RCT-156 | 1.1844106 | 0.9420644 | 0.9399793 | 1.0381737 | 0.9986454 | 1.1244341 | 1.0622237 |
| Ribosomal protein L13A | 1.0487988 | 1.0457848 | 1.286381 | 1.1408056 | 0.9728406 | 1.0602574 | 0.9763044 |
| Phase-1 RCT-258 | 1.0148119 | 0.9741302 | 1.0681762 | 0.9860126 | 1.0170202 | 0.9424304 | 0.9091457 |
| Insulin-like growth factor I | 0.9071596 | 0.8528556 | 0.8150179 | 1.1859654 | 1.1193604 | 1.1508737 | 0.8272933 |
| Cytochrome P450 3A1 | 0.3925169 | 0.9607139 | 0.6342781 | 0.9565345 | 1.3860658 | 1.324939 | 0.9093558 |
| {Ribosomal protein 16} | 0.930419 | 1.0100567 | 1.0367974 | 1.0250086 | 1.0438628 | 0.978285 | 1.0488125 |
| Organic cation transporter 3 | 0.8591428 | 1.0636623 | 1.0978192 | 1.0406392 | 1.0527525 | 0.9585852 | 0.8784431 |
| Calpain 2 | 0.933829 | 1.0178577 | 0.9760999 | 0.9417757 | 1.0243604 | 0.9639419 | 1.096472 |
| Phase-1 RCT-102 | 0.9256684 | 0.8741615 | 0.834073 | 0.9081425 | 0.9829737 | 0.9372578 | 0.9105169 |
| Equilbrative nitrobenzylthioinosine-sensitive | 0.7061874 | 0.8916178 | 0.9114829 | 0.9228234 | 0.947165 | 0.9992876 | 1.00976 |
| nucleoside transporter | | | | | | | |
| Phase-1 RCT-227 | 0.6957379 | 1.0906979 | 0.9052244 | 1.092621 | 1.0803473 | 0.9060479 | 1.1952057 |
| N-hydroxy-2-acetylaminofluorene sulfotransferase (ST1C1) | 0.6399825 | 0.8990871 | 0.9522612 | 0.9430827 | 1.1593462 | 1.0537323 | 1.0655216 |
| Cathepsin S | 0.8393835 | 0.9983161 | 0.8289139 | 0.7689259 | 0.8096504 | 0.8719899 | 1.0151666 |
| Apolipoprotein CIII | 1.0618973 | 1.1791219 | 1.1409535 | 0.9065248 | 1.0193423 | 0.8875141 | 0.977502 |
| Cytochrome P450 2A3 | 0.9616831 | 0.9240073 | 0.8881193 | 1.0183163 | 0.7661699 | 0.8518028 | 0.8927728 |
| Cytochrome P450 2C23 | 0.8748301 | 1.0480505 | 0.8232759 | 1.0420192 | 1.0558982 | 1.0696892 | 1.0559592 |
| Apolipoprotein C1 | 0.605068 | 0.7016537 | 0.9113973 | 1.0345109 | 1.002847 | 0.8613769 | 0.9054194 |
| Betaine homocysteine methyltransferase (BHMT) | 1.3380599 | 1.2377964 | 0.9175161 | 1.2336817 | 0.8187934 | 0.8016458 | 1.3018326 |
| Paraoxonase 1 | 0.7414972 | 0.8478844 | 0.7690818 | 0.9065319 | 0.937588 | 1.080976 | 0.959187 |
| Phase-1 RCT-207 | 1.2053261 | 1.2114515 | 0.9803304 | 1.0045472 | 1.0249842 | 0.9452272 | 0.996914 |
| Cathepsin B | 1.1328993 | 1.1619825 | 0.9424266 | 1.0691932 | 1.116098 | 1.1708552 | 0.9400481 |
| Phase-1 RCT-144 | 1.033265 | 0.9472964 | 1.010063 | 0.956241 | 0.9738656 | 0.9807126 | 0.9206791 |
| Alpha-1 microglobulin/bikunin precursor (Ambp) | 0.8408277 | 1.0909973 | 1.1167821 | 1.1625497 | 1.2028047 | 1.0130603 | 1.1407892 |
| Cyclin D1 | 0.8375268 | 0.898252 | 0.8412208 | 0.8218834 | 0.9682389 | 0.9620912 | 0.7229445 |
| Presenilin-1 | 0.923374 | 1.0545616 | 1.0521121 | 0.8568409 | 1.3601305 | 1.3173056 | 1.3232665 |
| Protein tyrosine phosphatase, receptor type, D | 0.8122816 | 0.8754633 | 0.8574596 | 1.0585487 | 1.045825 | 0.8708574 | 1.1217338 |
| Multidrug resistant protein-1 | 1.0241698 | 1.0195229 | 1.1085025 | 1.1043311 | 1.1114765 | 1.5355392 | 1.0328981 |
| Cathepsin L, sequence 2 | 0.9527039 | 1.0483936 | 1.0806347 | 1.0354873 | 1.0271924 | 1.2451468 | 1.1064475 |
| JNK1 stress activated protein kinase | 0.7613252 | 0.9381133 | 0.6988762 | 0.8284442 | 0.7118857 | 0.682459 | 1.112221 |
| Phase-1 RCT-43 | 1.2165002 | 1.0743041 | 1.1240122 | 1.0393558 | 0.9800261 | 1.089034 | 0.9829301 |
| Tissue plasminogen activator | 0.9561855 | 0.9272085 | 1.0160311 | 0.9865545 | 1.0425472 | 0.9399273 | 0.8905464 |
| Ubiquitin conjugating enzyme (RAD 6 | 0.9219541 | 1.0399281 | 1.1252346 | 0.9983574 | 1.1255294 | 0.982655 | 0.932471 |
| homologue) | | | | | | | |
| Phase-1 RCT-173 | 1.1017287 | 1.1501344 | 1.1152211 | 0.9731205 | 1.1828882 | 1.0616305 | 0.9605058 |
| Ornithine decarboxylase | 1.3267756 | 0.8327698 | 1.1366664 | 1.0894758 | 0.9187256 | 1.1856463 | 1.0970141 |
| Zinc finger protein | 0.9833363 | 1.0729427 | 1.0280254 | 1.0056078 | 1.0487918 | 1.0579873 | 0.9568813 |
| CDK108 | 0.9198505 | 1.06265 | 1.0953242 | 0.9206075 | 0.9425005 | 0.8619888 | 1.0373356 |
| Phase-1 RCT-117 | 1.3094491 | 1.5118849 | 1.4948909 | 1.3645344 | 1.3478218 | 1.0564127 | 1.0569247 |
| {Phase-1 RCT 98} | 1.0118723 | 1.072027 | 0.9325438 | 0.9473608 | 0.9694406 | 0.8656173 | 0.8784694 |
| Aquaporin-3 (AQP3) | 0.9686271 | 1.0059766 | 0.9218134 | 0.9285013 | 0.9998991 | 0.9228624 | 0.9684445 |
| Cholesterol esterase | 0.6157599 | 0.8567485 | 0.8809426 | 0.844155 | 1.2499719 | 0.9287296 | 0.9595349 |
| Ribosomal protein S17 | 0.7805191 | 0.8930048 | 0.939185 | 0.9590368 | 1.1023302 | 0.8861343 | 1.0372664 |
| Poly(ADP-ribose) polymerase | 1.1302532 | 1.0342768 | 1.1734868 | 1.0391626 | 0.997531 | 0.9721965 | 1.1117812 |
| Annexin V | 0.9189726 | 0.9559153 | 1.3144962 | 0.9965352 | 0.9840533 | 1.098367 | 0.9222842 |
| Phase-1 RCT-33 | 0.95493 | 0.9098275 | 1.0404282 | 1.1144661 | 0.7081248 | 0.8537341 | 0.900999 |
| Phase-1 RCT-61 | 1.4670745 | 1.0881618 | 1.3119195 | 1.036919 | 1.0348488 | 1.0725749 | 0.9478473 |
| Retinol dehydrogenase type III | 0.8233828 | 1.1329081 | 1.1148117 | 1.0863642 | 1.1140201 | 1.1240792 | 1.0093354 |
| ATPase inhibitor (rat mitochondrial IF1 protein) | 0.557388 | 0.8117026 | 0.7755773 | 0.9000158 | 0.96076 | 0.8885247 | 0.8720537 |
| Thymidylate synthase | 1.0118008 | 0.9592186 | 0.9419111 | 0.9946297 | 1.2154045 | 1.0566678 | 0.9017718 |
| Interleukin-18 | 0.9290031 | 1.030028 | 0.9996182 | 0.8702235 | 1.0327027 | 0.9210383 | 0.8179217 |
| Lecithin: cholesterol acyltransferase | 1.0268754 | 1.0609945 | 1.0068786 | 1.0860463 | 0.9450168 | 1.0919592 | 1.0269694 |
| Contrapsin-like protease inhibitor (CPi-21) | 0.9729078 | 0.9074876 | 1.1179221 | 1.2513523 | 1.3105705 | 1.1382114 | 0.8615128 |
| Proliferating cell nuclear antigen gene | 0.9365104 | 0.9523606 | 0.8949977 | 0.9196579 | 1.0295424 | 1.0174639 | 0.8652583 |
| Phase-1 RCT-230 | 0.9311258 | 1.0279263 | 1.0636965 | 0.838443 | 0.8645949 | 0.9119742 | 0.8886616 |
| Cytochrome P450 2D18 | 1.0390248 | 1.9042265 | 1.0754288 | 1.0737612 | 1.0519153 | 1.1882052 | 1.301183 |
| Phase-1 RCT-48 | 1.1899564 | 1.2670841 | 1.0539795 | 1.4486871 | 1.0513483 | 1.0282046 | 1.3260736 |
| Phase-1 RCT-292 | 1.0376343 | 1.044665 | 0.990129 | 1.0347557 | 0.9685224 | 0.961728 | 1.1928066 |
| Arginosuccinate synthetase 1 | 1.5379193 | 1.156935 | 1.2358634 | 1.2023311 | 0.7498106 | 0.742203 | 1.1898339 |
| C-reactive protein | 0.8982329 | 0.974672 | 0.9909916 | 1.0790579 | 1.0870641 | 0.8855426 | 1.2044594 |

| | STRZ | STRZ | STRZ | STRZ | STRZ | TAM |
|---|---|---|---|---|---|---|
| Compound-Dose | 20 | 20 | 75 | 75 | 75 | 50 |
| Animal Number | 1728 | 1729 | 1737 | 1738 | 1739 | 1447 |
| Liver Toxicity Necrosis Classification | no | no | no | no | no | no |
| Gene Name | | | | | | |
| Osteoactivin | 1.2243099 | 1.2246249 | 1.0846802 | 0.9574519 | 1.1001284 | 1.1097194 |
| Stathmin | 1.103538 | 1.0777308 | 0.9941534 | 0.8609514 | 0.8300368 | 0.8192716 |
| Calpactin I heavy chain | 1.3760517 | 1.0408756 | 1.0677018 | 0.9151135 | 0.9458525 | 1.0380106 |
| Phase-1 RCT-179 | 0.7269812 | 1.1213828 | 1.0825869 | 1.2493532 | 0.8517349 | 1.2327086 |
| Phase-1 RCT-107 | 1.4496994 | 1.1266361 | 1.251772 | 0.9089215 | 1.2208614 | 1.1506951 |
| Heme oxygenase | 1.0108157 | 0.7586112 | 0.8723247 | 0.7678056 | 0.7696845 | 1.0091981 |
| Collagen type II | 0.9237824 | 1.1678401 | 1.0942684 | 1.1594627 | 1.0919645 | 1.0110316 |
| Phase-1 RCT-81 | 1.197736 | 1.0085053 | 0.9978987 | 1.0666089 | 1.0332931 | 1.2653428 |
| Glycine methyltransferase | 1.2149953 | 1.6185144 | 0.9106805 | 1.6376675 | 3.1975884 | 1.1234487 |
| Cystatin C | 0.7723018 | 1.0771463 | 0.9264935 | 1.0528097 | 1.2012771 | 0.9273607 |
| Melanoma-associated antigen ME491 | 1.1674352 | 0.9813744 | 1.4166987 | 0.9826196 | 0.9821296 | 0.9183864 |
| Phase-1 RCT-146 | 1.4682918 | 0.9239754 | 0.8450403 | 0.5622723 | 0.8238125 | 1.2557349 |
| Gamma-actin, cytoplasmic | 0.717491 | 0.8677113 | 0.9429455 | 0.8279779 | 0.961109 | 0.8413016 |
| Adenine nucleotide translocator 1 | 0.5842528 | 0.7060467 | 0.8273255 | 0.9151545 | 0.7424973 | 0.7975672 |
| Thymosin beta-10 | 0.7805677 | 0.905291 | 0.8567076 | 0.9293428 | 0.8103728 | 0.7926056 |
| Insulin-like growth factor I, exon 6 | 0.6531724 | 1.1393111 | 1.2216674 | 1.384153 | 1.0300838 | 1.1137136 |
| Phase-1 RCT-78 | 1.0694075 | 1.119858 | 1.0477748 | 1.0511342 | 1.0335469 | 1 |
| Alpha-2-microglobulin | 0.5447074 | 1.0485274 | 0.9972873 | 1.3385065 | 1.2199463 | 0.9489955 |
| High affinity IgE receptor gamma chain | 1.0050336 | 0.9270305 | 1.1158305 | 1.0800115 | 0.9542303 | 0.9032592 |
| (FcERIgamma) | | | | | | |
| 60S ribosomal protein L6 | 0.6365804 | 0.7935085 | 0.9003246 | 0.8792679 | 0.6969312 | 1.1200544 |
| IgE binding protein | 1.2060109 | 0.9735124 | 1.0107667 | 0.955669 | 0.9408057 | 0.9952368 |
| Phase-1 RCT-149 | 1.1589658 | 1.1457653 | 1.1157316 | 1.2074043 | 1.2581483 | 0.9882579 |
| Cytochrome P450 2C11 | 0.8320344 | 0.9201089 | 0.6984519 | 0.8527751 | 0.9220443 | 1.8105075 |
| Uncoupling protein 2 | 1.248089 | 1.0079627 | 1.0264204 | 0.8022556 | 0.9914604 | 0.8838153 |
| Cofilin | 0.7707647 | 1.1284621 | 1.1046429 | 1.228446 | 0.8281398 | 0.7789225 |
| Alpha-tubulin | 0.8482105 | 0.9138956 | 0.8726882 | 0.8856475 | 0.9958774 | 0.812559 |
| Stearyl-CoA desaturase, liver | 0.4238961 | 0.601337 | 0.351504 | 0.561842 | 0.5289744 | 0.3743452 |
| Glutathione S-transferase theta-1 | 0.8867731 | 1.0399605 | 1.1985623 | 1.419214 | 1.1561726 | 0.8764995 |
| alpha-1,2-fucosyltrans-ferase | 0.7639767 | 0.7759068 | 0.9222957 | 0.8655344 | 1.1081831 | 1.3813348 |
| Phase-1 RCT-138 | 0.9917626 | 0.9614513 | 1.0408947 | 1.12392 | 1.0293933 | 0.9366307 |
| Phase-1 RCT-295 | 0.8069192 | 1.1551033 | 1.2538476 | 1.2769496 | 1.6037201 | 1.0451993 |
| Phase-1 RCT-164 | 0.9007969 | 0.8893122 | 0.4619913 | 0.8871523 | 0.8082184 | 0.9695705 |
| Alpha-2-macroglobulin, sequence 2 | 1.1320113 | 1.1148974 | 1.0450226 | 0.9621436 | 1.2281271 | 1.0839428 |
| Biliverdin reductase | 1.4070207 | 1.0098919 | 0.9844813 | 1.0074707 | 0.9906388 | 1.2151084 |
| Phase-1 RCT-121 | 1.0532557 | 0.9839483 | 0.8410542 | 0.8303798 | 0.7348307 | 1.0129507 |
| Dynamin-1 (D100) | 1.2564635 | 1.0753074 | 0.9846917 | 1.0697788 | 0.9902912 | 1.1671246 |
| Phase-1 RCT-192 | 0.7784321 | 1.1749935 | 1.2595668 | 1.0878023 | 0.9729429 | 0.9834151 |
| Fatty acid synthase | 0.8068292 | 0.8486362 | 0.4189462 | 0.7214631 | 0.6044751 | 0.5490401 |
| Phase-1 RCT-211 | 1.0862466 | 0.8936423 | 0.9874293 | 0.8917545 | 1.2570667 | 1.0998076 |
| Ribosomal protein S9 | 0.6152145 | 0.7712128 | 0.8963983 | 0.9071878 | 0.9110087 | 0.9955337 |
| NADP-dependent isocitrate dehydrogenase, | 0.6011269 | 0.9034246 | 0.9382711 | 1.0917659 | 0.7865609 | 0.9114295 |
| cytosolic | | | | | | |
| Phase-1 RCT-57 | 1.2077428 | 0.9882987 | 1.0076786 | 0.9593596 | 0.8439596 | 0.9368455 |
| Phase-1 RCT-36 | 1.1455652 | 0.9298433 | 0.8753593 | 0.982149 | 0.8733507 | 0.7828921 |
| Apolipoprotein All | 0.4591856 | 0.7694587 | 0.5248302 | 0.6477222 | 0.7374536 | 0.4371707 |
| Phase-1 RCT-68 | 1.2533047 | 1.0647455 | 1.1169829 | 1.0237498 | 1.12285 | 1.2534204 |
| Multidrug resistant protein-2 | 1.1542767 | 1.3571364 | 1.6923106 | 1.5719404 | 1.8383039 | 0.9097399 |
| Phase-1 RCT-39 | 1.0411807 | 1.031501 | 0.8240868 | 0.9467366 | 1.0361418 | 0.9921731 |
| NIPK | 1.212319 | 1.2366623 | 1.1536828 | 1.1322999 | 1.0431447 | 1.0086354 |
| F1-ATPase beta subunit | 0.6456343 | 0.9065758 | 1.2134236 | 1.2493954 | 1.313873 | 0.7734334 |
| Elongation factor-1 alpha | 0.535965 | 1.0601074 | 1.1061764 | 1.0798011 | 1.1194838 | 1.2044052 |
| Phase-1 RCT-24 | 0.9532853 | 1.19227 | 1.3435928 | 1.4411497 | 1.0845702 | 0.845805 |
| Beta-tubulin, class I | 0.9235838 | 1.2314029 | 1.5150888 | 1.5823231 | 1.1755879 | 0.7254335 |
| 14-3-3 zeta | 0.8351129 | 1.3160336 | 0.8426262 | 1.3229041 | 0.8558618 | 1.0741764 |
| Alpha-prothymosin | 0.6266907 | 1.0876229 | 0.9768357 | 1.2203887 | 0.6838969 | 0.7523959 |
| Phase-1 RCT-58 | 0.7601047 | 0.8208722 | 1.0336989 | 1.2482505 | 0.6407521 | 2.199484 |
| Phase-1 RCT-290 | 1.216917 | 0.8891509 | 0.8660626 | 0.7687791 | 1.053919 | 0.5628305 |
| Transitional endoplasmic reticulum ATPase | 0.7972375 | 1.1952288 | 1.0427264 | 1.3643146 | 1.0184547 | 0.8454454 |
| Beta-actin | 0.7766707 | 1.3493251 | 1.031318 | 1.1800225 | 1.041302 | 0.8188754 |
| Thioredoxin-1 (Trx1) | 0.6833471 | 0.8548299 | 0.9126272 | 0.9145474 | 1.0575657 | 1.167232 |
| Phase-1 RCT-174 | 1.3279877 | 0.9541307 | 0.9774008 | 0.7990093 | 0.7864783 | 0.7697521 |
| Beta-actin, sequence 2 | 0.6950991 | 1.213172 | 0.9740806 | 1.0384789 | 0.8151494 | 0.950477 |
| Phase-1 RCT-109 | 0.7466195 | 1.011249 | 1.1535703 | 0.6554295 | 0.919185 | 0.9970856 |
| Phase-1 RCT-154 | 1.0221314 | 0.9922736 | 0.8679116 | 1.0314462 | 0.8180467 | 0.8655247 |
| Hypoxanthine-guanine | 0.8808939 | 0.9667009 | 0.7686753 | 1.0247474 | 0.8022641 | 0.7501996 |
| phosphoribosyltransferase | | | | | | |
| Phase-1 RCT-213 | 1.0360441 | 1.0093895 | 1.0619203 | 1.0424808 | 0.8579894 | 0.8144141 |
| Phase-1 RCT-260 | 1.4263108 | 0.9901828 | 0.8820762 | 0.8781861 | 0.9603414 | 1.117077 |
| Phase-1 RCT-12 | 1.001223 | 1.5404131 | 1.3102182 | 1.3638963 | 1.2503355 | 0.8369378 |
| Phase-1 RCT-293 | 1.2253299 | 0.9824399 | 1.0404074 | 1.0336562 | 1.0002501 | 0.9802935 |
| Ribosomal protein S8 | 0.6296294 | 0.9370275 | 1.2287263 | 1.0598046 | 1.3393131 | 1.1447592 |
| Pyruvate kinase, muscle | 2.439358 | 0.9789357 | 1.1632777 | 1.0870942 | 1.2145262 | 0.8967425 |
| Nucleoside diphosphate kinase beta isoform | 0.7624617 | 0.9656604 | 1.3455538 | 1.2011223 | 1.11707 | 0.9557452 |
| p55CDC | 1.5314121 | 0.9231467 | 0.9917929 | 0.8388568 | 0.9412796 | 1.1426785 |
| Phase-1 RCT-156 | 0.6585947 | 1.0828391 | 1.0685302 | 1.0723513 | 1.3464487 | 0.9675014 |
| Ribosomal protein L13A | 0.6442303 | 1.1365991 | 1.0387917 | 1.2039374 | 0.8893468 | 1.1075616 |
| Phase-1 RCT-258 | 1.0584347 | 0.9934997 | 0.9664717 | 0.9439223 | 0.86119 | 0.8301703 |
| Insulin-like growth factor I | 0.4814515 | 0.7909744 | 0.9408259 | 0.8023364 | 0.857111 | 0.7364559 |
| Cytochrome P450 3A1 | 0.5025945 | 1.6286898 | 1.3637637 | 1.3501229 | 1.6831031 | 1.1539704 |
| {Ribosomal protein 16} | 0.6987189 | 1.0128479 | 1.218231 | 1.0891551 | 1.21882 | 1.1244115 |
| Organic cation transporter 3 | 1.1575669 | 0.8524823 | 0.8186814 | 0.6868284 | 0.6279067 | 1.1732975 |
| Calpain 2 | 1.1774316 | 1.063392 | 1.1755668 | 1.0184402 | 0.3159946 | 1.0436554 |
| Phase-1 RCT-102 | 0.7862169 | 1.052764 | 1.0383259 | 0.9736512 | 0.9444336 | 0.3936551 |
| Equilbrative nitrobenzylthioinosine-sensitive | 0.8774909 | 0.9818037 | 1.045352 | 1.0540717 | 1.3837552 | 1.2779714 |
| nucleoside transporter | | | | | | |
| Phase-1 RCT-227 | 0.9457889 | 1.3600143 | 1.4718487 | 1.6373597 | 1.455554 | 1.2004902 |
| N-hydroxy-2-acetylaminofluorene sulfotransferase (ST1C1) | 0.6829965 | 0.8853972 | 1.0481176 | 1.0879674 | 1.4004521 | 1.359605 |
| Cathepsin S | 0.8455864 | 0.8761063 | 1.0532115 | 0.9474124 | 0.9004108 | 0.7980229 |
| Apolipoprotein CIII | 0.7762484 | 0.8897858 | 0.7946594 | 0.8543825 | 0.7824168 | 0.763236 |
| Cytochrome P450 2A3 | 0.8806025 | 1.0009958 | 0.8361911 | 0.9072921 | 1.5547249 | 0.711871 |
| Cytochrome P450 2C23 | 0.4888257 | 0.9176694 | 0.705941 | 0.957059 | 0.6245154 | 1.0437006 |
| Apolipoprotein C1 | 0.5518714 | 0.8656991 | 1.1080223 | 1.2730874 | 1.0056074 | 0.6638314 |
| Betaine homocysteine methyltransferase (BHMT) | 0.7121674 | 0.7502647 | 0.7619162 | 0.680189 | 1.256894 | 0.388592 |
| Paraoxonase 1 | 0.4959338 | 0.8150122 | 1.0867616 | 1.0182929 | 1.1741096 | 0.924221 |
| Phase-1 RCT-207 | 1.0862797 | 1.0138552 | 0.9142367 | 0.977074 | 0.7682799 | 0.8988655 |
| Cathepsin B | 0.6852943 | 0.9141156 | 0.9783378 | 1.2194289 | 1.0277311 | 0.984421 |
| Phase-1 RCT-144 | 1.1007382 | 0.9527016 | 0.9518838 | 0.8999145 | 0.9336354 | 0.8637217 |
| Alpha-1 microglobulin/bikunin precursor (Ambp) | 0.5978957 | 0.9831958 | 1.0620427 | 1.2687117 | 1.2649356 | 1.35296 |
| Cyclin D1 | 0.8265954 | 0.8189215 | 0.9320683 | 1.2941844 | 1.5376081 | 1.0854852 |
| Presenilin-1 | 0.484315 | 1.2477808 | 1.2320577 | 1.3458834 | 0.7070613 | 0.984311 |
| Protein tyrosine phosphatase, receptor type, D | 0.4988497 | 0.9671001 | 0.9360541 | 1.0768951 | 1.1031088 | 0.7779771 |
| Multidrug resistant protein-1 | 1.1526083 | 1.3739319 | 1.7082675 | 1.6100783 | 1.8718935 | 1.0716873 |
| Cathepsin L, sequence 2 | 0.7247914 | 1.0071783 | 1.2597213 | 1.1047615 | 1.236479 | 0.7757663 |
| JNK1 stress activated protein kinase | 0.8714781 | 0.9520723 | 1.0123389 | 1.0133944 | 1.5970261 | 0.7966165 |
| Phase-1 RCT-43 | 0.6833943 | 0.9283462 | 0.8706233 | 1.0325038 | 0.8365396 | 0.8304468 |
| Tissue plasminogen activator | 1.2890865 | 0.8920277 | 0.9009835 | 0.7951425 | 0.8474498 | 0.7998607 |
| Ubiquitin conjugating enzyme (RAD 6 | 0.9839752 | 0.8841319 | 0.8349568 | 0.8375875 | 0.8277871 | 1.0226165 |
| homologue) | | | | | | |
| Phase-1 RCT-173 | 1.0653462 | 1.0307723 | 1.0381725 | 1.001258 | 0.7884076 | 0.7386677 |
| Ornithine decarboxylase | 1.4683063 | 1.13973 | 1.0931742 | 1.0918629 | 0.9304879 | 0.8926551 |
| Zinc finger protein | 1.1011443 | 1.027155 | 0.8855538 | 0.7641532 | 0.7463166 | 0.7126847 |
| CDK108 | 1.3546984 | 0.9100292 | 1.1718824 | 1.153268 | 1.1660123 | 1.5248212 |
| Phase-1 RCT-117 | 1.1379282 | 1.0188696 | 0.8059962 | 1.0740578 | 0.9854531 | 1.0851129 |
| {Phase-1 RCT 98} | 1.2580825 | 0.9845998 | 0.9924685 | 0.9605182 | 1.0268133 | 0.9774157 |
| Aquaporin-3 (AQP3) | 1.2247405 | 0.978438 | 0.9773159 | 0.90425 | 0.9489385 | 1.1723045 |
| Cholesterol esterase | 0.6810333 | 1.127382 | 1.3476512 | 1.4489516 | 1.0978162 | 0.5924354 |
| Ribosomal protein S17 | 0.5978484 | 0.871912 | 1.3727875 | 1.053949 | 1.415261 | 1.0143608 |
| Poly(ADP-ribose) polymerase | 1.2284114 | 1.3776127 | 0.9782076 | 0.9692534 | 0.9392816 | 0.9590664 |
| Annexin V | 1.1601754 | 1.0088307 | 0.9891409 | 0.6663262 | 0.9470701 | 1.1856428 |
| Phase-1 RCT-33 | 0.7302124 | 0.9235608 | 0.7809526 | 1.0752071 | 0.9178137 | 0.7096167 |
| Phase-1 RCT-61 | 1.1603876 | 0.9172168 | 0.8930378 | 0.8905908 | 0.9479443 | 1.0464458 |
| Retinol dehydrogenase type III | 0.620494 | 1.1201437 | 0.9638237 | 1.3935577 | 0.8439047 | 0.9883964 |
| ATPase inhibitor (rat mitochondrial IF1 protein) | 0.4915172 | 0.8199974 | 0.9257114 | 1.0413243 | 0.757646 | 0.7782897 |
| Thymidylate synthase | 1.1920702 | 0.925353 | 0.8912679 | 0.7942969 | 0.7462552 | 1.5781535 |
| Interleukin-18 | 1.5821973 | 0.8365701 | 0.776744 | 0.632142 | 0.6818047 | 1.3892351 |
| Lecithin: cholesterol acyltransferase | 0.6289421 | 0.9631896 | 1.0543888 | 1.3492266 | 0.8905896 | 1.030799 |
| Contrapsin-like protease inhibitor (CPi-21) | 0.4468347 | 0.8873984 | 1.0438302 | 1.1208612 | 1.1318318 | 0.5910108 |
| Proliferating cell nuclear antigen gene | 1.239071 | 0.9533623 | 0.9164068 | 0.7495655 | 1.0721165 | 1.1565125 |
| Phase-1 RCT-230 | 1.5155922 | 0.8725598 | 0.8174333 | 0.7342482 | 0.7955561 | 0.9776887 |
| Cytochrome P450 2D18 | 0.7957971 | 1.268232 | 1.6569328 | 1.7088546 | 1.2547759 | 1.0288837 |
| Phase-1 RCT-48 | 0.9708968 | 1.2679728 | 1.1506265 | 1.0921 | 1.3258321 | 0.9922454 |
| Phase-1 RCT-292 | 1.1432822 | 1.1017989 | 0.9590063 | 1.3243392 | 0.8971157 | 1.3037096 |
| Arginosuccinate synthetase 1 | 0.7241616 | 0.7928896 | 0.7619206 | 0.8802448 | 1.3320953 | 0.5502606 |
| C-reactive protein | 0.4286051 | 1.013201 | 0.8321757 | 1.003894 | 0.8920558 | 0.7495211 |
| | TAM | TAM | TAM | TAM | TAM | TET |
| Compound-Dose | 50 | 50 | 200 | 200 | 200 | 50 |
| Animal Number | 1448 | 1449 | 1457 | 1458 | 1459 | 128 |
| Liver Toxicity Necrosis Classification | no | no | no | no | no | no |
| Gene Name | | | | | | |
| Osteoactivin | 1.4593738 | 1.4354622 | 0.9156674 | 0.9271267 | 1.4489881 | 1.111706 |
| Stathmin | 0.8924632 | 1.0666918 | 1.0247312 | 0.9725609 | 0.8514158 | 0.7953818 |
| Calpactin I heavy chain | 1.240121 | 0.9778951 | 0.9969302 | 0.9557927 | 1.5026442 | 1.1654438 |
| Phase-1 RCT-179 | 1.200068 | 1.0308352 | 0.9532924 | 0.910473 | 1.5439694 | 1.5281963 |
| Phase-1 RCT-107 | 0.8184298 | 0.9168518 | 0.9745129 | 1.262224 | 0.6898616 | 0.6291339 |
| Heme oxygenase | 0.9926534 | 3.256237 | 0.9652817 | 0.7444853 | 1.9631225 | 1.4212245 |
| Collagen type II | 1.9059569 | 1.865747 | 1.0875052 | 1.0512567 | 2.6205215 | 1.3550191 |
| Phase-1 RCT-81 | 1.116063 | 1.1366917 | 1.2769817 | 1.1115655 | 1.1021906 | 1.2140749 |
| Glycine methyltransferase | 0.7449634 | 0.8546991 | 0.9416177 | 1.636076 | 0.5300259 | 0.5810583 |
| Cystatin C | 0.7986939 | 0.8205572 | 1.1487845 | 1.0577943 | 1.1383269 | 1.5704521 |
| Melanoma-associated antigen ME491 | 1.0253241 | 1.2379613 | 1.061879 | 1.0984194 | 1.5585691 | 1.7701731 |
| Phase-1 RCT-146 | 0.9993551 | 1.184361 | 0.9809233 | 1.0642656 | 1.0727811 | 0.9475151 |
| Gamma-actin, cytoplasmic | 0.9819978 | 1.0732131 | 0.9803544 | 1.03125 | 1.5400298 | 1.0568148 |
| Adenine nucleotide translocator 1 | 0.8037867 | 0.842804 | 1.0250198 | 0.9630055 | 1.3489165 | 1.3323421 |
| Thymosin beta-10 | 0.8731886 | 1.2343289 | 1.0576253 | 1.068372 | 0.8378507 | 0.6533549 |
| Insulin-like growth factor I, exon 6 | 1.430333 | 0.8133846 | 0.9964738 | 1.0385637 | 0.5989314 | 0.8256171 |
| Phase-1 RCT-78 | 0.8504709 | 0.8236732 | 0.9910051 | 0.9360749 | 1.121039 | 1.3304852 |
| Alpha-2-microglobulin | 0.7245219 | 0.2628816 | 0.6841449 | 0.863349 | 0.3571391 | 0.5107016 |
| High affinity IgE receptor gamma chain | 0.9723513 | 1.0183703 | 1.0212986 | 1.04375 | 1.3271794 | 1.1933444 |
| (FcERIgamma) | | | | | | |
| 60S ribosomal protein L6 | 1.1364577 | 1.2584774 | 1.3909622 | 1.1000795 | 1.3909442 | 1.3797946 |
| IgE binding protein | 1.3055383 | 1.6776943 | 1.0709163 | 1.1140306 | 1.3022168 | 1.0545791 |
| Phase-1 RCT-149 | 1.1078854 | 1.0999261 | 1.0461075 | 1.0888325 | 0.8836415 | 0.6558974 |
| Cytochrome P450 2C11 | 1.2024767 | 1.7224123 | 1.2299699 | 1.086355 | 0.8542774 | 0.800182 |
| Uncoupling protein 2 | 1.1049342 | 1.4715091 | 1.0067964 | 1.0594807 | 0.9206037 | 0.7332773 |
| Cofilin | 0.6940868 | 0.6178884 | 1.061151 | 0.9677419 | 1.5970074 | 1.7696434 |
| Alpha-tubulin | 0.9412674 | 0.9604809 | 0.9618555 | 0.9048713 | 1.355181 | 0.9701455 |
| Stearyl-CoA desaturase, liver | 0.2145357 | 0.2941856 | 0.9895397 | 0.868277 | 0.3678951 | 0.1376147 |
| Glutathione S-transferase theta-1 | 0.8059325 | 1.0211728 | 0.9414649 | 0.8757361 | 1.1329571 | 1.0846792 |
| alpha-1,2-fucosyltransferase | 1.2954609 | 1.2047058 | 1.0356114 | 0.973128 | 0.5549389 | 0.4950231 |
| Phase-1 RCT-138 | 1.2601048 | 1.1645329 | 0.9831732 | 1.0720646 | 1.1872942 | 1.1607027 |
| Phase-1 RCT-295 | 0.9835571 | 0.8780261 | 1.0431002 | 1.041891 | 1.0777891 | 0.9756405 |
| Phase-1 RCT-164 | 0.9378017 | 0.8318071 | 0.9516221 | 0.9483818 | 0.8725679 | 1.04261 |
| Alpha-2-macroglobulin, sequence 2 | 1.1581177 | 1.1781676 | 1.0801239 | 1.1468211 | 1.1666142 | 1.2004524 |
| Biliverdin reductase | 1.2435453 | 1.1774155 | 1.0320098 | 0.9432164 | 0.9711587 | 0.74546 |
| Phase-1 RCT-121 | 1.0763322 | 1.417324 | 0.9719241 | 0.9880465 | 1.2298164 | 0.7879077 |
| Dynamin-1 (D100) | 0.9740199 | 0.8722932 | 0.9822013 | 1.0236951 | 0.9733255 | 0.9612941 |
| Phase-1 RCT-192 | 0.9990223 | 1.4334843 | 1.0567214 | 1.0327778 | 1.2512774 | 1.0400229 |
| Fatty acid synthase | 0.4145703 | 0.3326824 | 0.9290772 | 0.7253708 | 2.1333337 | 0.836983 |
| Phase-1 RCT-211 | 1.1190886 | 0.9075859 | 0.820717 | 0.8216912 | 0.6430531 | 0.6151461 |
| Ribosomal protein S9 | 0.8570797 | 0.9586752 | 0.9373553 | 0.568662 | 1.3916678 | 1.1614269 |
| NADP-dependent isocitrate dehydrogenase, | 0.8650084 | 1.0354586 | 0.9922367 | 1.0104167 | 1.5450094 | 1.6075084 |
| cytosolic | | | | | | |
| Phase-1 RCT-57 | 0.8990986 | 0.8346534 | 0.9094566 | 0.9780928 | 0.6887604 | 0.7432425 |
| Phase-1 RCT-36 | 0.76851 | 0.9221356 | 0.9834945 | 0.2436913 | 0.8751487 | 0.865177 |
| Apolipoprotein AII | 0.4179815 | 0.8247424 | 0.841674 | 0.7469672 | 0.3369284 | 0.2961963 |
| Phase-1 RCT-68 | 1.0384897 | 1.0417048 | 1.0083302 | 1.0496398 | 1.1516235 | 1.1310896 |
| Multidrug resistant protein-2 | 0.8808942 | 1.2983185 | 1.1653787 | 1.1561671 | 0.783531 | 0.8906872 |
| Phase-1 RCT-39 | 1.1328313 | 1.4701704 | 0.9339386 | 0.9946808 | 1.1677458 | 1.2436255 |
| NIPK | 1.0800157 | 1.010523 | 0.9453876 | 0.9650822 | 0.8442537 | 0.7420584 |
| F1-ATPase beta subunit | 0.7234207 | 0.6974873 | 0.9473243 | 0.9601538 | 1.1889019 | 1.0715568 |
| Elongation factor-1 alpha | 1.1439074 | 1.1877654 | 1.0977062 | 0.9078364 | 1.2062154 | 1.0487126 |
| Phase-1 RCT-24 | 0.9162975 | 0.9225913 | 0.8968265 | 1.0231618 | 1.3247733 | 0.8326719 |
| Beta-tubulin, class I | 0.7904047 | 0.7769321 | 1.1179895 | 1.0411279 | 1.4156631 | 0.7321124 |
| 14-3-3 zeta | 1.0597755 | 1.1143247 | 1.0240328 | 1.0694444 | 1.0776157 | 0.7334917 |
| Alpha-prothymosin | 0.706495 | 0.5037957 | 1.0703942 | 0.9518485 | 1.1349918 | 1.4543841 |
| Phase-1 RCT-58 | 2.1073418 | 2.2197807 | 0.9506482 | 0.9681122 | 1.1481894 | 1.7373296 |
| Phase-1 RCT-290 | 0.8366842 | 0.5410875 | 0.8493593 | 1.0476191 | 0.6525731 | 0.4169931 |
| Transitional endoplasmic reticulum ATPase | 0.9168031 | 0.835853 | 1.014795 | 0.9607373 | 1.4361731 | 1.3731878 |
| Beta-actin | 0.773766 | 0.9419538 | 0.8470656 | 0.6997987 | 1.2042717 | 0.7800232 |
| Thioredoxin-1 (Trx1) | 0.9876392 | 1.1456518 | 1.0885688 | 0.8215975 | 0.7282571 | 0.7171875 |
| Phase-1 RCT-174 | 0.7203047 | 0.6816223 | 1.0908941 | 1.0398746 | 1.0105418 | 1.0419037 |
| Beta-actin, sequence 2 | 0.7893884 | 1.034017 | 0.8470524 | 1.0229468 | 0.5892997 | 0.594144 |
| Phase-1 RCT-109 | 1.1426961 | 0.8710962 | 1.2114725 | 1.1189235 | 0.6916003 | 0.593252 |
| Phase-1 RCT-154 | 0.839142 | 0.8650984 | 1.0661602 | 0.9418191 | 0.9916537 | 1.077727 |
| Hypoxanthine-guanine | 0.746833 | 0.8598034 | 0.86514 | 0.8060345 | 0.9251088 | 0.7473104 |
| phosphoribosyltransferase | | | | | | |
| Phase-1 RCT-213 | 1.0197107 | 0.8004347 | 1.0244253 | 0.9440789 | 1.1492091 | 0.9835206 |
| Phase-1 RCT-260 | 0.9081009 | 0.8575647 | 0.928856 | 0.9739584 | 0.9300445 | 1.0747064 |
| Phase-1 RCT-12 | 0.9451977 | 0.9426997 | 1.014945 | 0.9456751 | 0.8440369 | 0.7759596 |
| Phase-1 RCT-293 | 1.1117475 | 1.4295373 | 1.2611177 | 1.048655 | 1.4295901 | 1.1581489 |
| Ribosomal protein S8 | 1.0687653 | 1.04294 | 1.9830017 | 1.4413304 | 1.3358127 | 1.2842226 |
| Pyruvate kinase, muscle | 1.1414819 | 1.583705 | 1.1458491 | 1.0811069 | 0.859834 | 0.6784725 |
| Nucleoside diphosphate kinase beta isoform | 0.9911986 | 1.2780124 | 3.619473 | 1.1468894 | 1.1769778 | 1.0320437 |
| p55CDC | 1.1981819 | 1.3431047 | 1.163442 | 1.1482329 | 0.9509338 | 0.9757287 |
| Phase-1 RCT-156 | 1.1133417 | 0.8720493 | 1.1028656 | 1.0214286 | 0.9007809 | 0.9649864 |
| Ribosomal protein L13A | 1.1922925 | 1.1777829 | 1.2404103 | 0.9908006 | 0.8710556 | 0.6451947 |
| Phase-1 RCT-258 | 0.9573999 | 1.006238 | 1.0521591 | 0.8964089 | 1.0683368 | 1.0197595 |
| Insulin-like growth factor I | 0.9196208 | 0.640377 | 1.4861223 | 1.2721399 | 0.7747831 | 1.0408739 |
| Cytochrome P450 3A1 | 1.3922594 | 1.028873 | 0.8140411 | 0.8900496 | 0.527055 | 1.0569272 |
| &lcub;Ribosomal protein 16&rcub; | 1.1356888 | 1.2550375 | 1.519074 | 1.2920854 | 1.0949979 | 1.1968341 |
| Organic cation transporter 3 | 1.1519208 | 0.8883315 | 1.0956348 | 1.0445176 | 1.2626771 | 1.2327434 |
| Calpain 2 | 1.0522156 | 1.0738196 | 1.0276003 | 1.0685713 | 1.2477317 | 1.2163795 |
| Phase-1 RCT-102 | 0.4908548 | 0.4419672 | 0.9944223 | 0.9439189 | 0.5691357 | 1.0445414 |
| Equilbrative nitrobenzylthioinosine-sensitive | 1.0732262 | 0.9454078 | 0.9032345 | 1.0036765 | 0.9505073 | 1.3903188 |
| nucleoside transporter | | | | | | |
| Phase-1 RCT-227 | 0.9068688 | 0.853839 | 0.8773043 | 0.9489884 | 1.2714665 | 1.8463769 |
| N-hydroxy-2-acetylaminofluorene | 1.0204064 | 0.9319807 | 0.9796603 | 0.952206 | 1.0155205 | 1.5309821 |
| sulfotransferase (ST1C1) | | | | | | |
| Cathepsin S | 0.883688 | 1.7207108 | 1.0065002 | 1.0053391 | 1.401231 | 1.4726478 |
| Apolipoprotein CIII | 0.6747145 | 0.4788092 | 1.0736172 | 0.8787478 | 0.8709242 | 1.0050712 |
| Cytochrome P450 2A3 | 0.5535213 | 0.5123648 | 1.050471 | 1.2058908 | 0.8468744 | 0.8494669 |
| Cytochrome P450 2C23 | 0.9823846 | 0.6706258 | 0.8638299 | 0.9123831 | 1.0090829 | 1.5923204 |
| Apolipoprotein C1 | 0.666046 | 0.5292881 | 1.1346854 | 1.2577755 | 0.9300898 | 1.7913048 |
| Betaine homocysteine methyltransferase (BHMT) | 0.8091702 | 0.3756826 | 0.706817 | 1.1883947 | 0.500097 | 0.2574582 |
| Paraoxonase 1 | 0.7398151 | 0.6885805 | 1.2211512 | 1.1625702 | 1.2236048 | 2.1443892 |
| Phase-1 RCT-207 | 1.0170636 | 0.7756136 | 0.9676954 | 0.9742462 | 0.8716672 | 0.6995073 |
| Cathepsin B | 1.0621326 | 1.6336026 | 1.1190883 | 0.9642787 | 1.263461 | 1.2209727 |
| Phase-1 RCT-144 | 0.9066181 | 0.8628804 | 1.0231075 | 1.0179006 | 1.250104 | 1.0949695 |
| Alpha-1 microglobulin/bikunin precursor (Ambp) | 1.1373128 | 1.1302563 | 1.2879493 | 1.0719204 | 1.5047493 | 1.7301817 |
| Cyclin D1 | 0.9348403 | 0.613678 | 1.1158302 | 1.1004775 | 0.7450429 | 1.2645975 |
| Presenilin-1 | 1.3124993 | 0.4291233 | 0.9302051 | 0.7945359 | 1.4146115 | 2.113975 |
| Protein tyrosine phosphatase, receptor type, D | 0.746254 | 1.4961146 | 1.4479226 | 1.0701332 | 1.1051441 | 1.2954311 |
| Multidrug resistant protein-1 | 1.0822426 | 1.5798556 | 1.1182388 | 1.1844878 | 0.9501221 | 1.0411536 |
| Cathepsin L, sequence 2 | 0.7930164 | 1.4555985 | 0.9629704 | 0.9594445 | 1.0513943 | 1.0973378 |
| JNK1 stress activated protein kinase | 0.6660007 | 0.9165434 | 0.9272003 | 0.9620226 | 0.7173917 | 0.6581589 |
| Phase-1 RCT-43 | 0.9904685 | 0.8515441 | 0.9485259 | 0.9703776 | 0.9375008 | 1.0032583 |
| Tissue plasminogen activator | 0.8347278 | 0.7599084 | 0.9719241 | 1.0090725 | 1.0107998 | 0.9707525 |
| Ubiquitin conjugating enzyme (RAD 6 | 0.9337607 | 0.9840195 | 0.868466 | 0.6935573 | 1.3842158 | 1.1766691 |
| homologue) | | | | | | |
| Phase-1 RCT-173 | 0.8569775 | 0.7045154 | 0.9716809 | 0.9067029 | 0.589819 | 0.5698481 |
| Ornithine decarboxylase | 0.8333823 | 1.0389309 | 1.05509 | 1.0966463 | 1.4692414 | 0.943773 |
| Zinc finger protein | 0.8576682 | 1.114825 | 0.9673053 | 0.8956673 | 1.1451279 | 1.115625 |
| CDK108 | 0.7645742 | 0.770974 | 0.9296402 | 1.0672883 | 0.6341154 | 0.7153028 |
| Phase-1 RCT-117 | 0.8386941 | 0.9145227 | 0.9420843 | 0.9552393 | 0.9667149 | 0.8446661 |
| &lcub;Phase-1 RCT 98&rcub; | 0.8993151 | 0.6650417 | 1.0280149 | 1.0432065 | 1.0249015 | 1.1598634 |
| Aquaporin-3 (AQP3) | 0.981334 | 0.7740592 | 0.9893837 | 0.9996408 | 0.8864218 | 0.9307104 |
| Cholesterol esterase | 0.4491851 | 0.2911566 | 1.035995 | 0.8502219 | 0.5977835 | 0.9550474 |
| Ribosomal protein S17 | 0.9563243 | 0.9932396 | 1.7718953 | 1.4940748 | 1.3207366 | 1.3576534 |
| Poly(ADP-ribose) polymerase | 0.8262623 | 1.0765754 | 0.8901122 | 0.8488996 | 1.2909577 | 0.9896367 |
| Annexin V | 1.3045444 | 2.8090858 | 1.2514539 | 1.1848403 | 1.0540233 | 1.1994355 |
| Phase-1 RCT-33 | 0.6533312 | 0.8244256 | 1.0424186 | 1.2659005 | 1.003576 | 0.9908739 |
| Phase-1 RCT-61 | 1.2271369 | 1.2794726 | 0.9802228 | 0.9790576 | 0.9766795 | 1.1602639 |
| Retinol dehydrogenase type III | 0.939674 | 0.7824175 | 0.2867776 | 0.8637439 | 1.0727234 | 1.3407573 |
| ATPase inhibitor (rat mitochondrial IF1 protein) | 0.6504611 | 0.6779386 | 1.194452 | 1.0146837 | 0.810978 | 0.989843 |
| Thymidylate synthase | 1.2792563 | 1.2127564 | 1.0199203 | 1.0504441 | 1.0640273 | 1.0441935 |
| Interleukin-18 | 1.1416984 | 1.6292411 | 1.0214472 | 1.1393961 | 0.9290664 | 0.9758189 |
| Lecithin: cholesterol acyltransferase | 0.9665166 | 0.9265879 | 1.245112 | 0.966056 | 0.8678079 | 0.936558 |
| Contrapsin-like protease inhibitor (CPi-21) | 0.5887824 | 0.4351147 | 1.0161259 | 0.8858113 | 0.9802772 | 0.9550273 |
| Proliferating cell nuclear antigen gene | 1.0259466 | 1.1199329 | 1.029364 | 1.0256758 | 0.7944166 | 0.7591012 |
| Phase-1 RCT-230 | 1.2462212 | 0.991315 | 0.9962012 | 0.9852941 | 0.8204765 | 0.8869012 |
| Cytochrome P450 2D18 | 0.8333483 | 1.0300053 | 1.0558754 | 1.0578743 | 0.5751232 | 0.70266 |
| Phase-1 RCT-48 | 0.8423455 | 1.247472 | 0.7625309 | 0.8119437 | 0.9738718 | 0.8543581 |
| Phase-1 RCT-292 | 1.0804348 | 1.2679335 | 0.9502781 | 0.9318848 | 0.9942531 | 1.080346 |
| Arginosuccinate synthetase 1 | 0.5085136 | 0.5391616 | 0.8219738 | 0.9759047 | 0.6832215 | 0.8833464 |
| C-reactive protein | 0.6987783 | 1.5574603 | 1.2998095 | 0.9401329 | 1.0524763 | 1.2925609 |

| | TET | TET | TET | TET | THEO |
|---|---|---|---|---|---|
| Compound-Dose | 50 | 150 | 150 | 150 | 25 |
| Animal Number | 129 | 1247 | 1248 | 1249 | 2527 |
| Liver Toxicity Necrosis Classification | no | yes | yes | yes | no |
| Gene Name | | | | | |
| Osteoactivin | 0.9111013 | 0.8264334 | 1.0260454 | 1.0153564 | 0.9439873 |
| Stathmin | 0.8794609 | 0.9606721 | 0.871859 | 0.8696553 | 1.0473934 |
| Calpactin I heavy chain | 1.0076919 | 0.9583065 | 0.9567543 | 0.9762449 | 1.022197 |
| Phase-1 RCT-179 | 0.9411869 | 0.9324654 | 1.0861645 | 1.111791 | 0.7392051 |
| Phase-1 RCT-107 | 0.6865631 | 0.938266 | 0.8188751 | 0.9354374 | 1.0369701 |
| Heme oxygenase | 1.2008841 | 0.9821563 | 1.4299804 | 0.9186215 | 1.0238734 |
| Collagen type II | 1.0468239 | 0.6652665 | 1.0022603 | 1.0751145 | 1.8843336 |
| Phase-1 RCT-81 | 1.7022539 | 1.083994 | 1.066908 | 0.9635644 | 1.05673 |
| Glycine methyltransferase | 0.6080548 | 1.0713592 | 0.7147573 | 0.8613526 | 0.5229387 |
| Cystatin C | 1.5483481 | 1.35774911 | 1.121083 | 1.0634598 | 1.0357397 |
| Melanoma-associated antigen ME491 | 1.1240386 | 0.9734009 | 1.2863392 | 0.7899373 | 0.9671921 |
| Phase-1 RCT-146 | 0.9164777 | 0.8203623 | 0.987002 | 1.0537322 | 1.2048289 |
| Gamma-actin, cytoplasmic | 0.922807 | 0.7926624 | 0.7827887 | 0.7479537 | 0.8132638 |
| Adenine nucleotide translocator 1 | 0.9942057 | 1.157565 | 1.0466256 | 1.0019163 | 1.3132001 |
| Thymosin beta-10 | 0.9623967 | 0.8875138 | 0.8148084 | 0.9107285 | 1.2219738 |
| Insulin-like growth factor I, exon 6 | 0.9029239 | 1.0663493 | 1.0809072 | 1.160313 | 1.154791 |
| Phase-1 RCT-78 | 1.1084839 | 1.0097342 | 0.9380018 | 0.9547795 | 0.9876971 |
| Alpha-2-microglobulin | 0.9298546 | 1.525989 | 0.851433 | 0.9667409 | 0.3331187 |
| High affinity IgE receptor gamma chain | 1.0164474 | 0.9724725 | 1.0568936 | 0.9137461 | 0.8164414 |
| (FcERIgamma) | | | | | |
| 60S ribosomal protein L6 | 1.4439598 | 1.168827 | 1.0262003 | 1.0305756 | 0.6738533 |
| IgE binding protein | 0.9193306 | 0.7932342 | 0.9872382 | 0.9287917 | 0.9677091 |
| Phase-1 RCT-149 | 0.9817654 | 0.8018712 | 0.9316991 | 1.0175987 | 1.0295757 |
| Cytochrome P450 2C11 | 1.0480701 | 0.9774064 | 0.8246214 | 0.9859156 | 1.6314487 |
| Uncoupling protein 2 | 1.0830097 | 0.7135415 | 0.8558919 | 0.8822124 | 1.2355278 |
| Cofilin | 1.7500521 | 1.0061269 | 0.9496716 | 0.942413 | 0.8305797 |
| Alpha-tubulin | 0.976837 | 0.9539892 | 0.8337507 | 0.9535222 | 1.0399243 |
| Stearyl-CoA desaturase, liver | 0.7202163 | 0.8364046 | 0.4101887 | 0.5018216 | 0.630276 |
| Glutathione S-transferase theta-1 | 1.3034891 | 1.0981102 | 1.0568845 | 0.974996 | 0.9831837 |
| alpha-1,2-fucosyltransferase | 0.873508 | 1.04958011 | 1.0154439 | 0.9462336 | 1.4730015 |
| Phase-1 RCT-138 | 1.1745614 | 1.0392828 | 0.9730983 | 0.8700951 | 0.7398366 |
| Phase-1 RCT-295 | 1.1394249 | 1.1056099 | 1.0672175 | 1.055248 | 0.7257053 |
| Phase-1 RCT-164 | 1.0104914 | 0.8613547 | 1.0061858 | 0.9722011 | 1.3517094 |
| Alpha-2-macroglobulin, sequence 2 | 1.2767831 | 1.1435353 | 1.0814078 | 1.1816713 | 1.13102 |
| Biliverdin reductase | 1.08056 | 0.8489661 | 0.9123935 | 1.0531925 | 0.869605 |
| Phase-1 RCT-121 | 0.9539585 | 0.9411131 | 1.0112337 | 0.8958782 | 1.2320937 |
| Dynamin-1 (D100) | 0.9105951 | 1.064125 | 1.1034231 | 1.0238758 | 1.0144533 |
| Phase-1 RCT-192 | 0.9161504 | 0.7457357 | 0.9496058 | 0.9017777 | 1.0529741 |
| Fatty acid synthase | 0.6758497 | 0.5067422 | 0.365664 | 0.429894 | 0.9436995 |
| Phase-1 RCT-211 | 0.7787262 | 0.9421169 | 0.8800333 | 0.9759904 | 0.841068 |
| Ribosomal protein S9 | 1.2477274 | 0.9118408 | 1.1167902 | 0.9628038 | 0.8926329 |
| NADP-dependent isocitrate dehydrogenase, | 1.0858914 | 0.9045271 | 1.0331832 | 0.9170674 | 0.7841018 |
| cytosolic | | | | | |
| Phase-1 RCT-57 | 1.0549074 | 0.852848 | 0.7981179 | 0.8278081 | 1.1211863 |
| Phase-1 RCT-36 | 0.9044167 | 0.9790632 | 0.8661491 | 0.8464848 | 0.913539 |
| Apolipoprotein AII | 0.8484285 | 0.8772711 | 0.5781733 | 0.8311442 | 0.5910544 |
| Phase-1 RCT-68 | 1.0762966 | 1 | 1.0065248 | 0.9807117 | 1.0318869 |
| Multidrug resistant protein-2 | 1.350017 | 1.2071443 | 1.2934626 | 1.2931712 | 1.1018933 |
| Phase-1 RCT-39 | 0.9434533 | 0.965193 | 1.1390206 | 0.9912173 | 1.3652865 |
| NIPK | 0.8427751 | 0.8151875 | 1.0489025 | 1.1261688 | 1.0524813 |
| F1-ATPase beta subunit | 1.306525 | 0.9504593 | 0.9878199 | 0.921613 | 0.880668 |
| Elongation factor-1 alpha | 1.6899058 | 1.0361814 | 1.0235807 | 1.0069522 | 0.7180059 |
| Phase-1 RCT-24 | 0.9429513 | 0.8683687 | 0.9468863 | 0.8871929 | 1.2589827 |
| Beta-tubulin, class I | 1.2104839 | 0.9504175 | 0.819788 | 0.7366294 | 0.8227178 |
| 14-3-3 zeta | 1.0640305 | 0.8570824 | 1.0006263 | 1.1107637 | 0.7444238 |
| Alpha-prothymosin | 2.0749695 | 1.1065186 | 0.9365338 | 0.9492617 | 0.744374 |
| Phase-1 RCT-58 | 1.0463492 | 0.7849115 | 1.2055593 | 1.366229 | 0.6338905 |
| Phase-1 RCT-290 | 0.7927561 | 1.0671223 | 0.7354215 | 1.0087646 | 0.7680293 |
| Transitional endoplasmic reticulum ATPase | 1.0400572 | 1.0999343 | 1.0285616 | 1.0283169 | 0.9711154 |
| Beta-actin | 0.9357421 | 0.9706633 | 0.6144989 | 0.8456091 | 0.9032397 |
| Thioredoxin-1 (Trx1) | 0.9551379 | 1.1034807 | 0.9864799 | 0.940093 | 0.8395208 |
| Phase-1 RCT-174 | 1.4156057 | 1.0580946 | 0.9840148 | 1.0549314 | 1.0828202 |
| Beta-actin, sequence 2 | 0.8128897 | 1.0286019 | 0.8786379 | 0.9537994 | 1.3494225 |
| Phase-1 RCT-109 | 0.7499123 | 0.893597 | 0.7904146 | 0.9156843 | 1.2110715 |
| Phase-1 RCT-154 | 0.9997704 | 0.922269 | 0.9511279 | 0.9826002 | 0.9440532 |
| Hypoxanthine-guanine | 0.7851812 | 0.7247075 | 0.7778047 | 0.9265395 | 0.6419976 |
| phosphoribosyltransferase | | | | | |
| Phase-1 RCT-213 | 1.0496646 | 0.9421346 | 0.873502 | 0.9781903 | 0.8663518 |
| Phase-1 RCT-260 | 0.9206704 | 1.0886773 | 1.0692514 | 1.1889211 | 1.3925517 |
| Phase-1 RCT-12 | 1.0006022 | 1.0425255 | 1.0545737 | 1.0755978 | 1.0414817 |
| Phase-1 RCT-293 | 1.1877835 | 0.9795474 | 0.9179571 | 1.0324267 | 1.0200025 |
| Ribosomal protein S8 | 1.8202798 | 1.1880677 | 1.1329763 | 1.0218043 | 1.0495459 |
| Pyruvate kinase, muscle | 0.9130194 | 0.9712895 | 1.0607873 | 1.0701572 | 1.088908 |
| Nucleoside diphosphate kinase beta isoform | 1.1039991 | 0.7842448 | 1.0012261 | 1.0170344 | 1.2287678 |
| p55CDC | 1.0986667 | 0.987465 | 1.0401802 | 1.0425576 | 1.2779105 |
| Phase-1 RCT-156 | 1.1267285 | 0.862923 | 1.0271647 | 0.8827211 | 1.0982322 |
| Ribosomal protein L13A | 0.7612542 | 0.8892015 | 0.7492341 | 0.9109412 | 1.0774543 |
| Phase-1 RCT-258 | 1.0501573 | 1.0109632 | 0.9639279 | 1.0158812 | 1 |
| Insulin-like growth factor I | 1.581061 | 1.0367556 | 0.9590302 | 0.9112533 | 0.7377167 |
| Cytochrome P450 3A1 | 0.894108 | 1.4185355 | 1.2851051 | 1.3909551 | 0.3578462 |
| &lcub;Ribosomal protein 16&rcub; | 1.5310894 | 1.1205349 | 1.0460296 | 1.0043225 | 0.863389 |
| Organic cation transporter 3 | 1.0855058 | 1.1487874 | 1.0349265 | 1.0532159 | 0.9990547 |
| Calpain 2 | 0.925193 | 0.979009 | 1.0428138 | 0.973273 | 1.0005164 |
| Phase-1 RCT-102 | 0.9007846 | 0.9689459 | 0.9002422 | 0.9075255 | 0.7274006 |
| Equilbrative nitrobenzylthioinosine-sensitive | 0.9560939 | 1.2667282 | 1.1360657 | 1.1095122 | 0.8861675 |
| nucleoside transporter | | | | | |
| Phase-1 RCT-227 | 1.2574506 | 1.0245901 | 0.9471825 | 1.1129816 | 0.474617 |
| N-hydroxy-2-acetylaminofluorene | 0.9333333 | 1.0820472 | 1.1327052 | 1.0936708 | 0.6034035 |
| sulfotransferase (ST1C1) | | | | | |
| Cathepsin S | 0.9344577 | 0.7541388 | 1.0306507 | 0.9008671 | 0.6173464 |
| Apolipoprotein CIII | 1.3195881 | 0.9484063 | 0.8971593 | 1.0066559 | 0.5398277 |
| Cytochrome P450 2A3 | 0.9481984 | 1.3062721 | 1.2517292 | 0.8988619 | 1.0458453 |
| Cytochrome P450 2C23 | 1.2265053 | 0.721069 | 1.0147829 | 0.9343553 | 0.4930278 |
| Apolipoprotein C1 | 1.386585 | 0.9380705 | 0.9246763 | 0.8156323 | 0.7941084 |
| Betaine homocysteine methyltransferase (BHMT) | 0.7363536 | 1.0570852 | 0.7024548 | 1.0062121 | 0.4685615 |
| Paraoxonase 1 | 1.8155411 | 1.2592832 | 1.1042775 | 1.0238671 | 0.6066976 |
| Phase-1 RCT-207 | 0.950687 | 0.9699174 | 0.8670589 | 0.9824671 | 1.0335116 |
| Cathepsin B | 1.591015 | 1.0966926 | 0.9184436 | 0.9136755 | 0.5470927 |
| Phase-1 RCT-144 | 0.9697064 | 0.9641022 | 0.9434773 | 0.8873355 | 1.153113 |
| Alpha-1 microglobulin/bikunin precursor (Ambp) | 2.037957 | 1.0485048 | 1.0365838 | 0.907788 | 0.9093615 |
| Cyclin D1 | 1.5793718 | 1.0198611 | 1.1921045 | 0.9248729 | 0.9528638 |
| Presenilin-1 | 1.825675 | 1.3594123 | 1.2228777 | 0.9035695 | 0.7969697 |
| Protein tyrosine phosphatase, receptor type, D | 1.6857412 | 1.1559886 | 1.027252 | 0.8586587 | 0.5679001 |
| Multidrug resistant protein-1 | 1.3331015 | 1.3494302 | 1.2854148 | 1.2561107 | 1.0344088 |
| Cathepsin L, sequence 2 | 1.0037084 | 0.8284736 | 1.2413818 | 1.1347965 | 0.7556176 |
| JNK1 stress activated protein kinase | 0.9637426 | 0.9469242 | 0.7731888 | 0.9302982 | 0.5027485 |
| Phase-1 RCT-43 | 0.9828824 | 0.7129242 | 0.9867511 | 0.8976184 | 1.551694 |
| Tissue plasminogen activator | 0.9317702 | 0.9040229 | 0.9540729 | 0.9459493 | 1.0530041 |
| Ubiquitin conjugating enzyme (RAD 6 | 0.9601786 | 0.9699254 | 1.1419804 | 1.0497066 | 1.0781785 |
| homologue) | | | | | |
| Phase-1 RCT-173 | 0.9516354 | 1.0032647 | 0.8746993 | 0.9034601 | 1.0700729 |
| Ornithine decarboxylase | 0.9933765 | 0.9831134 | 0.9449068 | 1.1589149 | 0.8754736 |
| Zinc finger protein | 0.9926106 | 1.0491124 | 1.0424987 | 1.0412091 | 1.3729204 |
| CDK108 | 0.8812834 | 1.3615977 | 0.7651508 | 0.9485078 | 0.907906 |
| Phase-1 RCT-117 | 1.053611 | 0.9343635 | 0.8664106 | 1.2631993 | 0.9397426 |
| &lcub;Phase-1 RCT 98&rcub; | 0.9155555 | 0.888707 | 0.9337544 | 0.8911132 | 1.0080622 |
| Aquaporin-3 (AQP3) | 0.9224774 | 0.8364621 | 0.9926454 | 0.9600801 | 0.9209801 |
| Cholesterol esterase | 0.780546 | 1.133622 | 1.0753131 | 1.1480968 | 0.44411 |
| Ribosomal protein S17 | 1.1531806 | 1.2139524 | 1.1338438 | 0.9563918 | 0.9610105 |
| Poly(ADP-ribose) polymerase | 1.0727978 | 1.0088418 | 0.8888649 | 1.1209389 | 0.8667774 |
| Annexin V | 1.2911431 | 1.1367648 | 1.1267917 | 0.9663905 | 0.9036911 |
| Phase-1 RCT-33 | 0.9874969 | 0.9296394 | 0.7841324 | 0.8138803 | 0.7894672 |
| Phase-1 RCT-61 | 0.9235867 | 0.8542162 | 1.0485413 | 1.1045158 | 1.3368095 |
| Retinol dehydrogenase type III | 0.8930731 | 1.07267 | 0.9007706 | 0.9164097 | 0.9766471 |
| ATPase inhibitor (rat mitochondrial IF1 protein) | 1.4050406 | 0.7990843 | 0.8251641 | 0.8249721 | 0.6428102 |
| Thymidylate synthase | 0.9284331 | 1.1374989 | 1.0978152 | 1.0551666 | 0.969693 |
| Interleukin-18 | 0.9548661 | 1.0634431 | 1.1226108 | 1.072452 | 1.4854189 |
| Lecithin: cholesterol acyltransferase | 1.2490612 | 0.9588935 | 0.838668 | 0.9173787 | 0.62603 |
| Contrapsin-like protease inhibitor (CPi-21) | 1.2128198 | 1.0508431 | 1.0418751 | 1.1189729 | 0.5767232 |
| Proliferating cell nuclear antigen gene | 0.9536511 | 0.905197 | 1.0489548 | 1.0073432 | 0.8361839 |
| Phase-1 RCT-230 | 0.887592 | 0.9818298 | 1.0318762 | 0.9460511 | 1.4278322 |
| Cytochrome P450 2D18 | 1.1374121 | 0.7217983 | 0.6371558 | 0.7260739 | 0.5309774 |
| Phase-1 RCT-48 | 1.038529 | 1.0892698 | 0.919798 | 0.9770907 | 0.7874048 |
| Phase-1 RCT-292 | 0.9083062 | 0.9756478 | 0.9563883 | 0.9522948 | 0.8084324 |
| Arginosuccinate synthetase 1 | 0.796692 | 1.0394475 | 0.840268 | 0.7138395 | 0.7600619 |
| C-reactive protein | 1.6863599 | 1.1859386 | 1.241486 | 1.2206014 | 0.2513071 |
| | THEO | THEO | THEO | THEO | THEO |
| Compound-Dose | 25 | 25 | 100 | 100 | 100 |
| Animal Number | 2528 | 2529 | 2537 | 2538 | 2539 |
| Liver Toxicity Necrosis Classification | no | no | no | no | no |
| Gene Name | | | | | |
| Osteoactivin | 0.8655471 | 0.7951747 | 0.9994506 | 1.2981797 | 1.4004896 |
| Stathmin | 0.7306858 | 0.9803336 | 0.8978223 | 0.9138251 | 1.2253225 |
| Calpactin I heavy chain | 0.9487607 | 0.9281914 | 0.9534234 | 0.8219359 | 0.9716456 |
| Phase-1 RCT-179 | 0.7075132 | 1.0304589 | 1.117788 | 0.9127245 | 1.0997127 |
| Phase-1 RCT-107 | 1.0529628 | 0.7803582 | 0.9235434 | 0.8627719 | 0.9612151 |
| Heme oxygenase | 0.9599314 | 1.1038265 | 0.9061007 | 0.9629455 | 2.3488657 |
| Collagen type II | 1.3536807 | 1.251764 | 0.8906624 | 1.1519681 | 0.9217193 |
| Phase-1 RCT-81 | 1.0900862 | 1.0285593 | 0.9591064 | 1.2628028 | 1.3537611 |
| Glycine methyltransferase | 0.6572286 | 0.5909227 | 1.0470785 | 0.786464 | 0.902893 |
| Cystatin C | 1.2965426 | 1.2420752 | 0.8271145 | 0.8798389 | 0.894623 |
| Melanoma-associated antigen ME491 | 1.0277623 | 0.9946756 | 1.125158 | 0.9789107 | 1.1116495 |
| Phase-1 RCT-146 | 0.996079 | 1.131492 | 0.9878506 | 1.0637678 | 1.2291349 |
| Gamma-actin, cytoplasmic | 0.5515866 | 1.027878 | 0.8223015 | 0.8715668 | 0.9198636 |
| Adenine nucleotide translocator 1 | 0.8729637 | 0.782738 | 0.9707758 | 0.8348097 | 0.9175971 |
| Thymosin beta-10 | 0.5952493 | 1.2089348 | 0.7346243 | 0.6887959 | 0.9775659 |
| Insulin-like growth factor I, exon 6 | 1.0983737 | 1.395596 | 1.5775958 | 1.147367 | 0.7453213 |
| Phase-1 RCT-78 | 1.1144836 | 0.9393693 | 0.8901985 | 0.9074343 | 0.9343067 |
| Alpha-2-microglobulin | 0.5469285 | 0.427111 | 1.3203344 | 0.4680936 | 0.1667904 |
| High affinity IgE receptor gamma chain | 0.9539121 | 0.9090734 | 0.95691 | 0.8902521 | 1.0058405 |
| (FcERIgamma) | | | | | |
| 60S ribosomal protein L6 | 0.7008138 | 0.9665998 | 1.0452857 | 1.0040091 | 1.379938 |
| IgE binding protein | 0.9348605 | 0.9215376 | 0.9444792 | 1.0266514 | 1.5591253 |
| Phase-1 RCT-149 | 0.9837844 | 0.9470295 | 0.9743636 | 1.0691253 | 1.1294034 |
| Cytochrome P450 2C11 | 1.3571059 | 0.5251365 | 1.1388794 | 1.0354841 | 1.4142562 |
| Uncoupling protein 2 | 0.7179713 | 1.6212734 | 0.9974108 | 1.0232241 | 1.3016206 |
| Cofilin | 1.0147372 | 0.8048567 | 1.0416373 | 0.5264435 | 0.640332 |
| Alpha-tubulin | 0.6905199 | 1.1230869 | 0.7767149 | 0.9153561 | 1.0705589 |
| Stearyl-CoA desaturase, liver | 1.1157697 | 0.8775317 | 0.2534907 | 0.7077141 | 0.5177726 |
| Glutathione S-transferase theta-1 | 0.9270577 | 1.0920354 | 0.7282098 | 0.9495606 | 1.2030754 |
| alpha-1,2-fucosyltransferase | 1.0915514 | 1.3621341 | 1.1062969 | 1.361849 | 1.304925 |
| Phase-1 RCT-138 | 1.0567781 | 0.9487988 | 1.0744886 | 0.9949279 | 1.1903229 |
| Phase-1 RCT-295 | 0.6487687 | 0.8825836 | 0.8530932 | 0.8777504 | 0.9488451 |
| Phase-1 RCT-164 | 1.038816 | 1.0109447 | 1.0939037 | 0.8423506 | 0.801901 |
| Alpha-2-macroglobulin, sequence 2 | 1.3109968 | 1.1914955 | 0.9330702 | 1.0259082 | 1.1713793 |
| Biliverdin reductase | 0.9387116 | 1.2132981 | 0.9391783 | 1.1862991 | 1.4580367 |
| Phase-1 RCT-121 | 0.8341695 | 1.4746772 | 1.0190575 | 1.0194052 | 1.2408221 |
| Dynamin-1 (D100) | 1.1476438 | 0.9367628 | 1.0277789 | 1.0162876 | 0.9952914 |
| Phase-1 RCT-192 | 0.9553753 | 0.7700943 | 0.9543006 | 1.4717685 | 1.6335589 |
| Fatty acid synthase | 1.882626 | 1.2559593 | 0.3528421 | 0.731256 | 0.3082152 |
| Phase-1 RCT-211 | 0.6686353 | 0.8915187 | 0.9323607 | 0.8245868 | 0.8689761 |
| Ribosomal protein S9 | 0.9740033 | 1.0420064 | 0.9419319 | 0.8497704 | 1.1563432 |
| NADP-dependent isocitrate dehydrogenase, | 1.0194194 | 0.8815478 | 0.9936969 | 0.9294804 | 1.1469747 |
| cytosolic | | | | | |
| Phase-1 RCT-57 | 0.7411685 | 0.6531383 | 0.9230797 | 0.8748919 | 0.808885 |
| Phase-1 RCT-36 | 0.9742309 | 0.9274128 | 0.9833751 | 0.8977852 | 0.7931529 |
| Apolipoprotein AII | 0.3433045 | 0.75659 | 0.4438469 | 0.5425714 | 0.7554101 |
| Phase-1 RCT-68 | 0.8983781 | 1.0374647 | 1.0096682 | 1.0047882 | 1.0575044 |
| Multidrug resistant protein-2 | 0.9626233 | 0.9361247 | 1.0694923 | 1.1636235 | 1.2869731 |
| Phase-1 RCT-39 | 1.0004698 | 1.1249282 | 1.074314 | 1.2947693 | 1.1794333 |
| NIPK | 1.0016558 | 1.2817711 | 1.0060236 | 1.103139 | 0.916395 |
| F1-ATPase beta subunit | 1.0039136 | 0.9499114 | 0.9454686 | 0.7283472 | 0.7347103 |
| Elongation factor-1 alpha | 0.5796492 | 0.8220676 | 0.9983912 | 1.0934901 | 1.1863534 |
| Phase-1 RCT-24 | 1.2184496 | 1.0576416 | 0.7955263 | 0.8667344 | 1.0305177 |
| Beta-tubulin, class I | 0.6370407 | 0.7355633 | 0.6181679 | 0.6779704 | 0.793546 |
| 14-3-3 zeta | 0.577761 | 0.8150604 | 0.9858902 | 0.967304 | 1.0626543 |
| Alpha-prothymosin | 0.6815612 | 0.5585668 | 1.1078217 | 0.4709834 | 0.5235139 |
| Phase-1 RCT-58 | 0.3058283 | 1.0570168 | 2.1768947 | 1.1684563 | 2.1270645 |
| Phase-1 RCT-290 | 0.913735 | 0.63582 | 0.9041629 | 0.5143609 | 0.511731 |
| Transitional endoplasmic reticulum ATPase | 1.3344938 | 1.1264316 | 1.0387499 | 0.9458911 | 0.8629661 |
| Beta-actin | 0.1311615 | 0.8494183 | 0.7471425 | 0.8644844 | 0.8909391 |
| Thioredoxin-1 (Trx1) | 0.8738418 | 0.9292319 | 1.0727359 | 1.1470824 | 1.2429565 |
| Phase-1 RCT-174 | 1.1968094 | 1.0182481 | 1.0005869 | 0.8615401 | 0.8011578 |
| Beta-actin, sequence 2 | 1.0972925 | 1.1390389 | 1.1876013 | 1.1101569 | 0.9998226 |
| Phase-1 RCT-109 | 0.6313873 | 1.2473781 | 0.9232483 | 0.865861 | 0.9696283 |
| Phase-1 RCT-154 | 0.9147956 | 1.0487375 | 1.0351454 | 0.8838709 | 0.9492252 |
| Hypoxanthine-guanine | 0.6088522 | 1.0250294 | 0.9299498 | 0.8140731 | 0.8679831 |
| phosphoribosyltransferase | | | | | |
| Phase-1 RCT-213 | 0.7172951 | 1.0241956 | 0.9896202 | 0.7695257 | 0.7774004 |
| Phase-1 RCT-260 | 1.1186496 | 1.0727962 | 1.027492 | 0.9970643 | 0.876902 |
| Phase-1 RCT-12 | 1.0848569 | 1.0748717 | 0.9487128 | 1.0477113 | 0.9499872 |
| Phase-1 RCT-293 | 1.001201 | 0.8838134 | 0.9790437 | 1.2189043 | 1.2828721 |
| Ribosomal protein S8 | 1.0555949 | 1.0569206 | 1.0523475 | 0.9060161 | 1.1816401 |
| Pyruvate kinase, muscle | 0.8300554 | 0.9623874 | 0.9813544 | 1.0311542 | 1.3139831 |
| Nucleoside diphosphate kinase beta isoform | 0.9819934 | 0.7904844 | 0.9899326 | 1.0604186 | 1.3778458 |
| p55CDC | 1.132794 | 1.2729814 | 0.9585072 | 1.1473416 | 1.4029422 |
| Phase-1 RCT-156 | 1.1043661 | 1.487789 | 1.0174173 | 1.0235306 | 0.9845364 |
| Ribosomal protein L13A | 0.4741493 | 1.3689916 | 0.9912565 | 1.0162365 | 1.218399 |
| Phase-1 RCT-258 | 0.8824961 | 1.0191926 | 1.0486726 | 1.0537394 | 0.9556746 |
| Insulin-like growth factor I | 0.6117908 | 0.8691485 | 1.1197673 | 0.7439446 | 0.6710431 |
| Cytochrome P450 3A1 | 1.0022085 | 0.3829207 | 1.2261665 | 0.9611545 | 0.9105874 |
| &lcub;Ribosomal protein 16&rcub; | 0.7575327 | 0.964162 | 1.0615621 | 1.0048615 | 1.3763187 |
| Organic cation transporter 3 | 0.8587449 | 0.9020348 | 1.0038176 | 1.05086 | 1.3869711 |
| Calpain 2 | 1.0243666 | 1.0559239 | 0.9635661 | 0.972679 | 1.0597734 |
| Phase-1 RCT-102 | 1.251003 | 0.8768043 | 0.7049941 | 0.608601 | 0.5071515 |
| Equilbrative nitrobenzylthioinosine-sensitive | 1.073528 | 0.8245471 | 1.0557333 | 1.0269713 | 0.863256 |
| nucleoside transporter | | | | | |
| Phase-1 RCT-227 | 0.72932 | 0.6952624 | 1.075881 | 0.9383043 | 0.9918468 |
| N-hydroxy-2-acetylaminofluorene | 0.9727364 | 0.6543506 | 1.1881144 | 0.9627996 | 0.7668895 |
| sulfotransferase (ST1C1) | | | | | |
| Cathepsin S | 0.81014 | 0.6929922 | 0.7496163 | 0.7817898 | 1.2957855 |
| Apolipoprotein CIII | 0.5240913 | 1.0778145 | 1.0837048 | 0.6867876 | 0.4220249 |
| Cytochrome P450 2A3 | 1.1756643 | 0.8239003 | 1.158736 | 0.4606828 | 0.5629744 |
| Cytochrome P450 2C23 | 0.6752747 | 0.6961997 | 1.1085724 | 0.8367928 | 0.634084 |
| Apolipoprotein C1 | 1.1396707 | 0.8654589 | 0.9507824 | 0.5439099 | 0.3983358 |
| Betaine homocysteine methyltransferase (BHMT) | 0.6386545 | 0.5661774 | 0.366287 | 0.3719672 | 0.4001266 |
| Paraoxonase 1 | 1.0078434 | 0.7086661 | 0.953878 | 0.7145455 | 0.7609299 |
| Phase-1 RCT-207 | 0.9430994 | 1.0022372 | 0.9752834 | 0.743731 | 0.7557015 |
| Cathepsin B | 0.7443042 | 0.773631 | 0.9877188 | 1.189265 | 1.6034989 |
| Phase-1 RCT-144 | 1.2845808 | 1.1830295 | 0.9312338 | 0.9509848 | 0.9626842 |
| Alpha-1 microglobulin/bikunin precursor (Ambp) | 1.0852538 | 0.9571683 | 1.1587236 | 1.2596275 | 1.3297168 |
| Cyclin D1 | 1.3603672 | 1.1007365 | 1.3525175 | 0.8982188 | 0.7241426 |
| Presenilin-1 | 1.713635 | 0.9057966 | 1.4446639 | 0.9570869 | 0.5024484 |
| Protein tyrosine phosphatase, receptor type, D | 0.8420785 | 0.8158035 | 0.7756056 | 0.8954672 | 1.310383 |
| Multidrug resistant protein-1 | 0.9505376 | 0.8092104 | 1.0296369 | 1.2397505 | 1.4541641 |
| Cathepsin L, sequence 2 | 0.5783056 | 0.9195674 | 1.0192608 | 0.9393339 | 1.2920045 |
| JNK1 stress activated protein kinase | 0.6040309 | 0.5702724 | 0.9863885 | 0.9187029 | 1.0015393 |
| Phase-1 RCT-43 | 1.0393059 | 1.3995354 | 1.1037642 | 1.075554 | 0.9238983 |
| Tissue plasminogen activator | 0.981935 | 0.8913652 | 0.9855442 | 0.6722749 | 0.6891344 |
| Ubiquitin conjugating enzyme (RAD 6 | 0.9899912 | 1.0402838 | 0.9566788 | 0.8937411 | 1.1082156 |
| homologue) | | | | | |
| Phase-1 RCT-173 | 0.7674911 | 1.122707 | 0.8849879 | 0.6844239 | 0.6756755 |
| Ornithine decarboxylase | 0.714929 | 0.7554051 | 0.8943428 | 0.7807609 | 0.9389691 |
| Zinc finger protein | 0.8851261 | 0.6589912 | 0.9031268 | 1.0496286 | 1.0951006 |
| CDK108 | 0.9870803 | 0.9064974 | 1.1564429 | 0.8395077 | 0.8282324 |
| Phase-1 RCT-117 | 0.907521 | 0.9056203 | 0.9198586 | 0.9440017 | 0.8829084 |
| &lcub;Phase-1 RCT 98&rcub; | 1.0190796 | 0.9087404 | 0.9278135 | 0.7410806 | 0.6535163 |
| Aquaporin-3 (AQP3) | 1.0895181 | 1.0166655 | 0.9392362 | 0.8242095 | 0.8241132 |
| Cholesterol esterase | 1.584328 | 0.8032869 | 0.7886507 | 0.4640237 | 0.2294444 |
| Ribosomal protein S17 | 0.93107 | 1.08941 | 0.9409508 | 0.8881791 | 1.0974076 |
| Poly(ADP-ribose) polymerase | 0.8536922 | 1.0932785 | 0.8945006 | 1.037515 | 1.1394837 |
| Annexin V | 0.8812172 | 1.0095917 | 1.0267067 | 1.3376569 | 2.9444597 |
| Phase-1 RCT-33 | 0.8258869 | 0.9451405 | 0.8229727 | 0.8209217 | 0.7265351 |
| Phase-1 RCT-61 | 1.0526901 | 1.1015881 | 1.2135288 | 0.9586231 | 1.2494681 |
| Retinol dehydrogenase type III | 0.8667759 | 0.9493734 | 1.0538102 | 0.8216207 | 0.8187126 |
| ATPase inhibitor (rat mitochondrial IF1 protein) | 0.8621808 | 0.8386619 | 0.908902 | 0.6816437 | 0.6439863 |
| Thymidylate synthase | 1.0918785 | 1.0197808 | 0.9955753 | 1.1757237 | 1.2238994 |
| Interleukin-18 | 1.2105834 | 1.1293201 | 1.034199 | 1.0437804 | 1.3318069 |
| Lecithin: cholesterol acyltransferase | 0.5657091 | 0.6882092 | 1.1377075 | 0.8260798 | 0.9438108 |
| Contrapsin-like protease inhibitor (CPi-21) | 0.4871935 | 0.6906781 | 0.6789883 | 0.3222411 | 0.2395016 |
| Proliferating cell nuclear antigen gene | 0.9432957 | 1.1492062 | 1.0431465 | 1.0610026 | 1.3164668 |
| Phase-1 RCT-230 | 0.9798917 | 1.3452677 | 0.99246 | 0.9930898 | 1.0305598 |
| Cytochrome P450 2D18 | 0.4336672 | 0.584147 | 1.0165128 | 0.9329941 | 0.9621029 |
| Phase-1 RCT-48 | 0.8480614 | 0.8795712 | 0.7131599 | 1.2703255 | 1.5181451 |
| Phase-1 RCT-292 | 0.9873539 | 1.0418901 | 0.9795494 | 1.2376605 | 1.2966425 |
| Arginosuccinate synthetase 1 | 1.1158245 | 0.8622381 | 0.5674616 | 0.5047304 | 0.5549544 |
| C-reactive protein | 0.4425917 | 0.3153853 | 0.8507592 | 0.9469596 | 1.4076234 |

(1) Gene expression data for 72 hour timpoint are presented as mean ratio of treatment/controlfor all 72 hour predictive genes (Table 26).
(2) Compound and dose abbreviations as in Table 1
(3) Individual animal number
(4) Liver necrosis classification for compound-dose group at 72 h: yes, necrosis observed; no, no necrosis observed
(5) Predictive gene (as in Table 26 and as included in Table 32)

What is claimed is:

1. A method of predicting the liver toxicity in an individual to an agent comprising the steps of:
obtaining a biological sample from the individual treated with the agent;
measuring the expression of one or more liver toxicity predictive genes in the sample, wherein the genes are selected from the group consisting of partial gene sequences of genes identified as responsive to agents causing liver necrosis, thereby generating a test expression profile; and
using the test expression profile with a set of reference expression profiles in a Predictive Model to determine whether the agent will induce liver toxicity in the individual.

2. The method according to claim 1, wherein the liver toxicity predictive genes are selected from the group of partial gene sequences listed in Table 32 that represent 24 hour combo All genes.

3. The method according to claim 2, wherein the partial gene sequences correspond to rat genes.

4. The method according to claim 2, wherein the partial gene sequences correspond to dog genes.

5. The method according to claim 2, wherein the partial gene sequences correspond to non-human primate genes.

6. The method according to claim 2, wherein the partial gene sequences correspond to human genes.

7. The method according to claim 1, wherein the liver toxicity predictive genes are selected from the group of partial gene sequences listed in Table 32 that represent 24 hour combo 2 genes.

8. The method according to claim 7, wherein the partial gene sequences correspond to rat genes.

9. The method according to claim 7, wherein the partial gene sequences correspond to dog genes.

10. The method according to claim 7, wherein the partial gene sequences correspond to non-human primate genes.

11. The method according to claim 7, wherein the partial gene sequences correspond to human genes.

12. The method according to claim 1, wherein the liver toxicity predictive genes are selected from the group of partial gene sequences listed in Table 32 that represent 24 hour Combo 5 genes.

13. The method according to claim 12, wherein the partial gene sequences correspond to rat genes.

14. The method according to claim 12, wherein the partial gene sequences correspond to dog genes.

15. The method according to claim 12, wherein the partial gene sequences correspond to non-human primate genes.

16. The method according to claim 12, wherein the partial gene sequences correspond to human genes.

17. A method of predicting the liver toxicity of an agent using an in vitro system, comprising the steps of:
obtaining a biological sample from an in-vitro cultured cells or explants, treated with the agent;
measuring the expression of one or more liver toxicity predictive genes in the sample, wherein the genes are selected from the group consisting of partial gene sequences of genes identified as responsive to agents causing liver necrosis, thereby generating a test expression profile; and
using the test expression profile with a set of reference expression profiles in a Predictive Model to determine whether the agent will induce liver toxicity in the individual.

18. The method according to claim 17, wherein the liver toxicity predictive genes are selected from the group of partial gene sequences listed in Table 32 that represent 24 hour combo All genes.

19. The method according to claim 18, wherein the partial gene sequences correspond to rat genes.

20. The method according to claim 18, wherein the partial gene sequences correspond to dog genes.

21. The method according to claim 18, wherein the partial gene sequences correspond to non-human primate genes.

22. The method according to claim 18, wherein the partial gene sequences correspond to human genes.

23. The method according to claim 17, wherein the liver toxicity predictive genes are selected from the group comprising of 24 hour Combo 2 genes.

24. The method according to claim 23, wherein the partial gene sequences correspond to rat genes.

25. The method according to claim 23, wherein the partial gene sequences correspond to dog genes.

26. The method according to claim 23, wherein the partial gene sequences correspond to non-human primate genes.

27. The method according to claim 23, wherein the partial gene sequences correspond to human genes.

28. The method according to claim 17, wherein the liver toxicity predictive genes are selected from the group of partial gene sequences listed in Table 32 that represent 24 hour Combo 5 genes.

29. The method according to claim 28, wherein the partial gene sequences correspond to rat genes.

30. The method according to claim 28, wherein the partial gene sequences correspond to dog genes.

31. The method according to claim 28, wherein the partial gene sequences correspond to non-human primate genes.

32. The method according to claim 28, wherein the partial gene sequences correspond to human genes.

33. A process for predicting the liver toxicity in a biological sample from an individual, an in-vitro cell cultures or explants to an agent via a programmable machine, the process comprising the steps of:
obtaining a biological sample treated with the agent;
measuring the expression of one or more liver toxicity predictive genes in the sample, wherein the genes are selected from the group consisting of partial gene sequences of genes identified as responsive to agents causing liver necrosis, thereby generating a test expression profile; and
using the test expression profile with a set of reference expression profiles in a Predictive Model to determine whether the agent will induce liver toxicity in the individual.

34. A computer program product for enabling a computer to perform Predictive Model analysis for liver toxicity on a biological sample from an individual, an in-vitro cell cultures or explants to an agent, the computer program product comprising:
software instructions for enabling the computer to perform predetermined operations, and a computer readable medium embodying the software instructions;
The pre-determined operations comprising:
measuring an expression of one or more liver toxicity predictive genes in a sample, wherein the genes are selected from the group consisting of partial gene sequences of genes identified as responsive to agents causing liver necrosis, thereby generating a test expression profile; and
using the test expression profile with a set of reference expression profiles in a Predictive Model to determine whether the agent will induce liver toxicity in the individual.

35. A Computer system adopted to predict liver toxicity in a biological sample from an individual, an in-vitro cell cultures, or explants to an agent, comprising a processor and a memory including software instructions adapted to enable the computer system to perform operations comprising:
measuring the expression of one or more liver toxicity predictive genes in the sample, wherein the genes are selected from the group consisting of partial gene sequences of genes identified as responsive to agents causing liver necrosis, thereby generating a test expression profile; and
using the test expression profile with a set of reference expression profiles in a Predictive Model to determine whether the agent will induce liver toxicity in the individual.

36. A computer program product for predicting liver toxicity from a test sample expression profile, comprising:
an encrypted training data set;
encrypted lists of genes selected from genes predictive of liver toxicity to be used with the encrypted training data set, and
a Predictive Model that uses the encrypted training data sets, the encrypted lists of genes, and the test sample expression profile to predict the liver toxicity of the test sample.

37. The computer program product of claim 36, wherein the encrypted lists of genes are selected from any Combination Category appearing in Tables 5, 20 and 26.

38. The computer program product of claim 36, wherein the encrypted lists of genes comprise a 24 hour Combo All genes as set in Table 5.

39. The computer program product of claim 36, wherein the encrypted lists of genes comprise a 6 hour Combo All genes as set in Table 20.

40. The computer program product of claim 36, wherein the encrypted lists of genes comprise a 72 hour Combo All genes as set in Table 26.

41. A method for mining genes predictive for liver toxicity, comprising the steps of:
collecting expression levels of a plurality of candidate toxicity predictive genes among a multiplicity of samples;
defining a group of samples to be a training set;
defining another group of samples to be a test set;
optionally generating additional training and test sets; and
selecting a set of genes which are predictive of liver toxicity based on evaluating the training and test sets in a Predictive Model.

42. The method according to claim 41, wherein the expression levels are stored as a database on an electronic medium.

43. An integrated system for predicting liver toxicity, comprising:
means for measuring gene expression profiles of genes predictive of liver toxicity from biological samples exposed to a test agent; and
a computer system operably linked to the means wherein the computer system is capable of implementing a Predictive Model.
